(19) United States
(12) Patent Application Publication
(10) Pub. No.: US 2026/0062450 A1
(43) Pub. Date: Mar. 5, 2026

(54) RECOMBINANT AAVS WITH IMPROVED TROPISM AND SPECIFICITY

(71) Applicant: AFFINIA THERAPEUTICS INC., Waltham, MA (US)

(72) Inventors: Charles Francis ALBRIGHT, Boston, MA (US); Kevin C. OLIVIERI, Boston, MA (US); Xiaohong CAO, Southborough, MA (US); Jie TAN, Somerville, MA (US)

(21) Appl. No.: 19/104,215

(22) PCT Filed: Aug. 17, 2023
(86) PCT No.: PCT/US2023/072424

§ 371 Date: Feb. 15, 2025

Related U.S. Application Data
(60) Provisional application No. 63/371,743, filed on Aug. 17, 2022.
(60) Provisional application No. 63/371,744, filed on Aug. 17, 2022.
(60) Provisional application No. 63/371,715, filed on Aug. 17, 2022.
(60) Provisional application No. 63/482,754, filed on Feb. 1, 2023.
(60) Provisional application No. 63/482,758, filed on Feb. 1, 2023.
(60) Provisional application No. 63/482,776, filed on Feb. 1, 2023.
(60) Provisional application No. 63/502,577, filed on May 16, 2023.
(60) Provisional application No. 63/502,525, filed on May 16, 2023.
(60) Provisional application No. 63/502,595, filed on May 16, 2023.

(51) Int. Cl. C07K 14/005; C12N 15/86

(52) U.S. Cl. CPC C07K 14/005; C12N 15/86; C12N 2750/14122; C12N 2750/14143

(57) ABSTRACT

The present disclosure provides a modified AAV capsid protein comprising a targeting peptide in variable region VIII (VR, VIII) and/or a peptide segment in variable region I (VR I). The modified AAV capsid protein can form an rAAV, which has a preferred tropism, specificity or biodistribution in vivo or in vitro. The rAAV of the present disclosure can be used for gene therapies targeted at a specific tissue.

### 1. CROSS-REFERENCE TO RELATED APPLICATIONS

This application claims the benefit of U.S. Provisional Application Nos. 63/371,715 filed Aug. 17, 2022; 63/371,743 filed Aug. 17, 2022; 63/371,744 filed Aug. 17, 2022; 63/482,754 filed Feb. 1, 2023; 63/482,758 filed Feb. 1, 2023; 63/482,776 filed Feb. 1, 2023; 63/502,525 filed May 16, 2023; 63/502,577 filed May 16, 2023, and 63/502,595 filed on May 16, 2023, each of which is hereby incorporated by reference in its entirety.

### 2. SEQUENCE LISTING

The instant application contains a Sequence Listing, which has been submitted as an XML file on triplicate CD-Rs (labeled as CRF, Copy 1, and Copy 2) and mailed on Aug. 17, 2023 via USPS Priority Mail Express; and is hereby incorporated by reference in its entirety. Said XML file, created on Aug. 15, 2023, is named 53019WO_CRF_sequencelisting.XML, and is 137,114,243 bytes in size.

### 3. BACKGROUND

Adeno-associated virus (AAV) has become the vector system of choice for in vivo gene therapy. A growing variety of recombinant AAVs (rAAVs) engineered to deliver therapeutic nucleic acids have been developed and tested in nonhuman primates and humans, and the FDA has recently approved two rAAV gene therapy products for commercialization.

Although AAV vectors are safer and less inflammatory than other viruses, toxicities have occurred following administration of high doses of rAAVs for gene therapy. Thus, local administration of rAAVs to a target tissue or organ has been used to improve targeting and reduce systemic toxicity. Further, various natural and synthetic AAV variants have been tested to develop an AAV vector with desired tropism and specificity.

In general, the capsid is thought to be the primary determinant of infectivity and host-vector related properties such as adaptive immune responses, tropism, specificity, potency, and bio-distribution. Indeed, several of these properties are known to vary between natural serotypes and engineered AAV variants. Over the last decade, novel synthetic AAV variants have been developed by using a variety of capsid engineering techniques, one of which is the insertion of small, 7 amino acid-long, peptides into an exposed loop of the capsid protein, called variable region VIII (VRVIII). In some circumstances, the insertion of a novel peptide into a wild type capsid changes the tropism of the variant. For example, insertion of a peptide having the sequence RGDLGLS (SEQ ID NO: 156) into the capsid of AAV9 was found to increase infection of astrocytes (see PHD thesis of Eike Kienle, Ruprecht-Karls-Universitat Heidelberg, 2014) and primary breast cancer cells (Michelfelder et al. (2009)).

To date, however, there is little understanding as to how these changes on the capsid functionally alter these properties. Additionally, AAV vectors with a desired tropism and specificity to common therapeutic targets, such as muscles, have not yet been available.

### 4. SUMMARY

The present disclosure provides a modified AAV capsid protein that can form an rAAV having a preferred tropism and specificity to a therapeutic target. In some embodiments, a modified adeno-associated virus (AAV) capsid protein with the preferred tropism comprises (i) a targeting peptide at a site within variable region VIII (VR VIII); or (ii) a peptide segment within variable region I (VR I), wherein the targeting peptide has a sequence of X₁X₂X=RGDX₇X₈X₉X₁₀, wherein X₁, X₂, X₃, X₇, X₈, X₉ and X₁₀ are independently selected from any amino acid residue, wherein the peptide segment has an amino acid sequence of P₁P₂P₃P₄P₅P₆P₇P₈NDNP₁₂ and P₁, P₂, P₃, P₄, P₅, P₆, P₇, P₈, and P₁₂ are independently selected from any amino acid residue. In some cases, the modified AAV capsid protein comprises both (i) the targeting peptide and (ii) the peptide segment. In some cases, the AAV capsid protein comprises the targeting peptide but not the peptide segment. In some cases, the AAV capsid protein comprises the peptide segment but not the targeting peptide.

Applicant demonstrated an ability to modify AAV capsid proteins with a targeting peptide selected from SEQ ID NO. 1, and SEQ ID NOs.: 238-44858, in the VR VIII. In particular, the modified AAV capsid protein can include a targeting peptide selected from SEQ ID NOs.: 44859-44879, 44881-44883, 44911, 44912, 44913, and 44918-44919 in the VR VIII region. The resulting rAAVs containing the modified AAV capsid protein having a targeting peptide at a site within variable region VIII (VR VIII) demonstrated better targeting with more specific expression in muscle tissue.

Additionally, Applicant demonstrated an ability to optimize the modified AAV capsid protein by identifying which amino acid modifications at which residues (or combinations of amino acid modifications at various residues) in VR I decrease liver tropism and increase muscle tropism (or do not effect reduced muscle tropism as compared to a control capsid, such as the reference capsid). Applicant previously demonstrated that a liver-toggle mutation is associated with liver-on or liver-off tropism (i.e. G266A). Applicant now reports a modified AAV capsid protein comprising one, two, three or more amino modifications (within the variable region I (VR I)) that either alone or together provide synergistic effects to the specific targeting of an rAAV to a target tissue. Therefore, the modified AAV capsid proteins of the present disclosure can change the tropism, specificity and/or bio-distribution of an AAV comprising the modified AAV capsid protein.

Overall, the resulting rAAVs containing the modified AAV capsid protein comprising (i) a targeting peptide at a site within variable region VIII (VR VIII); and (ii) a peptide segment within variable region I (VR I) demonstrated better targeting with more specific expression of a transgene in the target tissue, e.g., muscles, and also exhibited reduced liver tropism when systemically administered to a mammalian subject.

The use of AAV for gene therapy for muscular disorders (e.g., XLMTM) has been limited because of liver toxicity. Modified AAV capsid proteins provided herein provide an improved way to treat the diseases with better safety. The modified AAV capsid proteins could deliver a construct encoding a therapeutic gene (e.g., MTM1) with reduced liver tropism and/or improved muscle tropism. Additionally, the construct could drive higher and more specific MTM1 expression at the target by virtue of appropriate expression regulatory elements (ERE) (e.g., promoter sequences) and/or codon optimized coding sequences.

In one aspect, this disclosure features a modified adeno-associated virus (AAV) capsid protein, comprising: a targeting peptide within VR VIII; and a peptide segment within VR I, wherein the targeting peptide has a sequence of X₁X₂X₃RGDX₇X₈X₉X₁₀, wherein X₁, X₂, X₃, X₇, X₈, X₉ and X₁₀ are independently selected from any amino acid residue, and wherein the peptide segment has an amino acid sequence of P₁P₂P₃P₄P₅P₆P₇P₈NDNP₁₂ and P₁, P₂, P₃, P₄, P₅, P₆, P₇, P₈, and P₁₂ are independently selected from any amino acid residue.

In another aspect, the present disclosure features a modified adeno-associated virus (AAV) capsid protein comprising: a sequence of a reference AAV capsid protein comprising: (i) a targeting peptide at a site within variable region VIII (VR VIII) of the reference AAV capsid protein; and (ii) one or more modifications to comprise a peptide segment within variable region I (VR I) of the reference AAV capsid protein, wherein the targeting peptide has a sequence of X₁X₂X₃RGDX₇X₈X₉X₁₀, wherein X₁, X₂, X₃, X₇, X₈, X₉ and X₁₀ are independently selected from any amino acid residue, wherein the peptide segment has an amino acid sequence of P₁P₂P₃P₄P₅P₆P₇P₈NDNP₁ and P₁, P₂, P₃, P₄, P₅, P₆, P₇, P₈, and P₁₂ are independently selected from any amino acid residue.

In some embodiments, the modified AAV capsid protein described herein comprises: (i) the targeting peptide within VR VIII; and (ii) the peptide segment within VR I

In some embodiments, the targeting peptide does not comprise RGDLLLS (SEQ ID NO: 1).

In some embodiments, the peptide segment does not comprise an alanine (A) at P₆ and a threonine (T) at P₈.

In some embodiments, the targeting peptide does not comprise RGDLLLS (SEQ ID NO: 1); and the peptide segment does not comprise an alanine (A) at P₆ and a threonine (T) at P₈.

In some embodiments, the modified AAV capsid protein has one or more modifications comprising amino acid insertions, deletions, substitutions, or combinations thereof as compared to a reference AAV capsid protein.

In some embodiments, the modified AAV capsid protein comprises one or more modifications comprising an amino acid insertion, deletion, substitution, or a combination thereof as compared to a reference AAV capsid protein to introduce the targeting peptide within VR VIII. In some embodiments, the modified AAV capsid protein also includes more modifications outside of the VR I and VR VIII of the reference AAV capsid protein.

In some embodiments, the modified AAV capsid protein has at least 90%, 95%, 98%, 99% or 99.5% sequence identity to the sequence of the reference AAV capsid protein.

In some embodiments, the modified AAV capsid protein is selected from VP1, VP2 and VP3.

In some embodiments, the reference AAV capsid protein is a capsid protein of an AAV variant selected from the group consisting of: AAV9; Anc8065; Anc80-55; Anc80-129; Anc80-156; Anc80-751; Anc80-1029; Anc80-1712; AAV2; AAV1; AAV6; AAV3; AAV LK03; AAV7; AAV8; AAV hu.37; AAV rh.10; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu.9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L33; Anc80L36; Anc80L44; Anc80L1; Anc110; and Anc80DI.

In some embodiments, the reference AAV capsid protein is a capsid protein having a sequence selected from SEQ ID Nos: 54-152, 44885-44898, 44916-44917, or a fragment thereof.

In some embodiments, the reference AAV capsid protein is a capsid protein having a sequence of SEQ ID NO: 61 or a fragment thereof.

In some embodiments, the reference AAV capsid protein is a capsid protein having a sequence of SEQ ID NO: 132 or a fragment thereof.

In some embodiments, the reference AAV capsid protein is a capsid protein having a sequence of SEQ ID NO: 142 or a fragment thereof.

In some embodiments, the reference AAV capsid protein is a capsid protein selected from: Anc80-55, Anc80-129, Anc80-156, Anc80-751, Anc80-1029, and Anc80-1712.

In some embodiments, the reference AAV capsid protein is a capsid protein having a sequence selected from SEQ ID NOs: 44885-44898, and 44916-44917.

In some embodiments, the AAV capsid protein has the sequence selected from SEQ ID NOs.: 44900-44909.

In some embodiments, X₇, X₈, X₉ and X₁₀ are independently selected from A, D, E, F, G, H, I, K, L, N, Q, R, S, T, V, and Y.

In some embodiments, X₁, X₂, and X₃ are independently selected from any amino acid residue.

In some embodiments, X₁, X₂, and X₃ are amino acids identical to the amino acids at corresponding positions of the reference AAV capsid protein.

In some embodiments, X₁ is an amino acid that is identical to the amino acid at a corresponding position of the reference AAV capsid protein.

In some embodiments, X₂ is an amino acid that is identical to the amino acid at a corresponding position of the reference AAV capsid protein.

In some embodiments, X₃ is an amino acid that is identical to the amino acid at a corresponding position of the reference AAV capsid protein.

In some embodiments, X₁ and X₃ are amino acids that are identical to the amino acids at corresponding positions of the reference AAV capsid protein.

In some embodiments, X₁ and X₂ are amino acids that are identical to the amino acids at corresponding positions of the reference AAV capsid protein.

In some embodiments, X₂ and X₃ are amino acids that are identical to the amino acids at corresponding positions of the reference AAV capsid protein.

In some embodiments, X₁, X₂, and X₃ are amino acids that are not identical to the amino acids at corresponding positions of the reference AAV capsid protein.

In some embodiments, X₁ is an amino acid that is not identical to the amino acid at a corresponding position of the reference AAV capsid protein.

In some embodiments, X₂ is an amino acid that is not identical to the amino acid at a corresponding position of the reference AAV capsid protein.

In some embodiments, X₃ is an amino acid that is not identical to the amino acid at a corresponding position of the reference AAV capsid protein.

In some embodiments, X₁ and X₃ are amino acids that are not identical to the amino acids at corresponding positions of the reference AAV capsid protein.

In some embodiments, X₁ and X₂ are amino acids that are not identical to the amino acids at corresponding positions of the reference AAV capsid protein.

In some embodiments, X₂ and X₃ are amino acids that are not identical to the amino acids at corresponding positions of the reference AAV capsid protein.

In some embodiments, the modified AAV capsid protein comprises one or more substitutions, one or more insertions, one or more deletions, or a combination thereof into VR VIII of the reference AAV capsid protein. In some embodiments, one or more modifications comprises a substitution of one or more amino acids between amino acid positions 565 and 595 of the reference AAV capsid.

In some embodiments, X₁ is selected from S, E, A, D, N, Q, or T.

In some embodiments, X₁ is D or E. In some embodiments, X₁ is S, A or T. In some embodiments, X₁ is S, A or E.

In some embodiments, X₂ is selected from N, A, D, E, F, G, H, I, K, L, M, N, P, Q, R, S, T, V, W, or Y.

In some embodiments, X₂ is selected from K, E, D, A, S, F, N, V or L. In some embodiments, X₂ is selected from K, E or D. In some embodiments, X₂ is selected from N, A or Y. In some embodiments, X₂ is selected from N, Y or S.

In some embodiments, X₃ is selected from R, Q, A, D, E, F, G, H, I, K, L, M, N, P, S, T, V, W, or Y.

In some embodiments, X₂X₃ is NR.

In some embodiments, X₁X₂X₃ is ENR.

In some embodiments, X₁X₂X₃ is SNR.

In some embodiments, X₁X₂X₃ is selected from the group consisting of: DII, DWM, EEI, DML, DWI, SLE, EIN, NHE, DFI, EEL, TEQ, TDA, EDT, NEV, TDW, QFE, EDY, DTT, EPL, SEN, SEQ, TAE, EVN, ELN, DVQ, ETI, EVI, ESV, ETW, SEW, DNW, EVF, EAW, EPF, EIY, EIF, EPY, DVI, DMM, DQI, DHL, DTL, DVL, NDL, DLL, DMQ, NEF, DFL, DIM, TEW, DYI, SDY, DYY, DHF, DKE, DTW, DTI, ELY, TEY, TEI, DAI, DQY, DMY, EWG, DMV, DMI, EPH, QEG, DIN, NEI, EYY, DIV, SEG, DVG, DYQ, EGF, NDI, EGY, DVF, DVH, DGF, DIY, DSF, DGW, EHY, DRE, TEH, DTS, NEN, NEM, NEH, TEN, DSN, DVT, DQS, DKD, DTH, DVV, DQK, NET, DKP, TEV, NDS, QET, EVL, SET, SDT, AEQ, QEF, SEY, SEF, SPF, EGQ, ETH, TDQ, QEA, QDQ, AEN, ESS, NDT, EFM, EFI, EHM, DFM, QDT, SME, DYT, EHV, ENV, EAV, EAI, ESI, DAT, ENQ, EAM, ADN, EFQ, SDS, TDH, SDH, DAS, TWE, SSF, DRD, EFL, TDF, QDA, EMH, SGE, AEW, DAH, TET, TDM, TNE, SAE, NSE, SFE, QDI, DSA, ADV, SEI, AEI, QDV, ADT, DNM, DNQ, ADL, TDL, SDL, SDM, TDV, DNI, DHY, DTY, DAA, DSY, QLY, DVM, DAY, DMT, DQT, DAQ, DTV, DSH, QDF, DST, DNL, DSI, DFV, DNY, DAF, DKI, DKF, DTM, DSL, NDV, TDI, DSV, DAV, DKV, DAM, DNV, DKM, DKL, DKW, DSM, ENI, SDI, DKT, QGE, NNE, QYW, AGM, ESH, QDH, QEH, DYH, ENS, DKQ, TGE, DSS, EST, DSQ, DNS, EFV, SWE, DKN, DKS, SEH, EAL, EAS, ADQ, SDQ, ADI, SDV, EAH, ASE, EAF, EYV, DKA, SNE, AGE, EGV, EQF, ETF, EVY, EQY, QEY, QEN, SAD, AEF, EMF, SDF, ADF, ADY, AVF, TLY, SIY, SLY, AIY, DYV, AEY, ENF, EMY, QDY, ETY, EAY, TVY, SVY, QVF, QVY, TMY, DKY, ALY, AVY, TMW, EQW, SPW, SIW, DTQ, TFF, AMF, EYT, DRV, SPY, SIF, SLF, ENY, EWY, EYI, EFY, TLF, TVF, TYY, TFY, SVF, SYW, TAW, SAW, TIY, NIM, NFM, NII, NIV, SII, SIV, SEM, TTI, NSY, SSY, SNY, SEV, AEH, ALT, SGQ, QTY, SYY, AMY, QMY, SGF, SGY, ALF, TTF, SFN, QAF, ASF, SAF, TAF, TAY, TGL, SAY, QAY, ADH, ASI, SSV, SSI, TGV, SGV, DPH, TEG, DLY, ESE, TEF, NPE, DHA, DLI, DWG, EWM, EET, EIL, TEL, DYL, EMW, EVW, EPV, EDV, SLD, EGH, QRY, TDG, SQE, ELH, EYW, EMM, EFF, QEI, EQG, DVS, ESP, EIQ, EPM, EPQ, SPE, EPN, NQD, ELQ, TDT, ELT, EPI, ETM, ETG, EHI, EMI, EIT, ETV, EVV, EVM, TPY, QEW, EIW, EPT, EIH, DFY, DQL, NDA, DIW, TDY, ELF, DVY, ESG, EPG, EIG, ELG, EVG, DHI, EIS, DYM, DFT, DFH, EQS, TPF, EEF, DWN, SDN, EYL, AET, DMN, QSP, DNA, DHM, DHT, EQI, ESN, EYF, EHF, NAD, AEG, AES, TEM, SEL, DYS, EVH, EAN, EGL, TFE, ETL, TYE, SHE, ELM, TYD, SYE, ESA, EFT, EAT, AMD, AND, DMS, QMD, SLQ, EYS, QDN, EYQ, EKD, QYD, QFD, DER, AFD, EAA, EFS, DHV, QSE, TSD, SFD, DGY, TWD, QWD, EHL, QGD, QSD, AWD, AGD, QIY, EMQ, EAG, ERD, DDR, DAL, QDL, DNT, EGN, ENH, NGE, DYN, DNH, DNF, ESQ, ESM, ESL, SSD, ENL, EMV, QDS, TSE, SWD, ASD, ENT, SSE, EGM, EGS, EGT, EGI, AIF, NIN, NIQ, NYW, EPW, EGG, TIW, QIW, TTW, EKW, NLY, SVM, TPT, AAW, AIH, TIH, QGT, QFF, QYF, QFY, EHH, QLH, TYF, STY, DRL, EYM, SFF, QLF, QIF, APF, TYW, QII, QPF, SPI, TIL, SVL, NVI, SIM, SLL, QEL, AEM, AEL, QEV, QDM, NDM, SYL, ASL, TPL, ANI, NML, TIM, AEV, TMI, SWI, SFI, SNW, TNF, NAF, ASP, QSQ, APY, SFM, TAN, QAW, SGS, SGH, TGF, QSN, ERY, QMN, TGQ, NGM, NGV, AMN, ALN, QFV, AAF, TFV, TSY, EDK, ASN, SAV, QSV, QGV, QST, ASM, TAI, QSM, QSL, QSI, SSL, AGV, TGT, AGL, QGM, TGM, SGM, SGI, THD, QVE, ALE, QLE, EWP, AFP, AEE, ADE, NIE, TYP, QIE, TFP, SYP, ELW, DVW, NEW, EPA, EVA, TLE, AWE, NME, QME, ATE, AYE, DOM, TPD, DFG, QPE, EDI, EWL, ELL, QPD, EWF, ALD, SVD, QVD, ADA, APD, AHE, EQL, DTA, AFE, DAW, QAE, TME, STE, THE, QID, AAE, EIA, EML, SDA, SIE, TQE, TVD, NWE, NOE, EYG, QYP, EIV, EII, QFP, TID, EQN, EVS, TYG, EAQ, ELV, QLD, TLD, QQD, TIN, TLN, SPN, TPN, NFD, NEY, DSW, NMD, DGA, DYA, DIH, DNP, DQW, DQV, EFA, EPS, DEN, DWV, ELI, DTF, EQT, DHW, ESF, DEK, DMH, EWS, EHA, TTD, EWI, QLQ, SQD, QHD, NSD, EMA, TND, ATD, DGS, TAD, TMD, QES, AHD, QAD, DGH, DGL, NTE, ENG, SMD, NDQ, EMS, ENA, EMT, EYN, DDK, EWA, EWQ, EFH, QWE, DGT, DGM, EWV, EWH, EWT, QND, DGV, ESY, AAD, SND, ANE, TIG, QIG, AVD, SFP, AWP, QDW, SLW, TPA, AII, TIV, SWP, AFY, APN, AVN, TVN, TIS, TIQ, TLQ, AIQ, QVN, NFY, QFG, EMN, NVF, SYF, SFW, TPQ, AIL, TWI, QWN, TWN, QWQ, AQF, ASA, TWV, EWN, TMF, NGI, ATN, SAS, TMQ, TMN, AYN, TQF, SMF, QMQ, SMQ, QAN, AAY, SFQ, TYQ, TFQ, TWQ, AVI, and SGL.

In some embodiments, X₁X₂X₃ is selected from the group consisting of: APW, TEL, TDA, QPY, SPN, EHY, DWK, DLK, DFK, DVK, NSI, DIR, SPF, SEL, DRT, DRF, ADL, TDL, SDL, DNY, DKI, NDV, DKM, DNH, DNF, DSS, EST, EWT, DKN, DKS, SEH, ESQ, ESL, QND, EAH, AIF, AVF, QVF, TMY, ALY, NNG, NIF, NTF, NFF, AWF, NPY, SWF, AII, AYF, AQW, NFY, AGP, QQF, TKE, TNG, NSF, NAW, QAG, ERG, NKD, QSG, QNG, EAK, QWF, SWY, TFF, TYF, NYY, QFG, NWA, AMF, STY, TNW, ANW, AWM, TSF, DRL, DRV, SPY, NVF, SIF, QLF, SLF, QIF, SYF, APF, AGF, SVF, SAW, TIY, DHK, DAK, EGK, DYK, QNL, QPL, SPV, NPM, STL, NIL, AGA, NTV, SQA, QQA, NNF, NWL, NMA, NNA, NHN, NFH, ELR, NYM, ERA, NFS, SRD, NMM, NNL, NNQ, NNS, QDR, SDK, NNI, AQH, ANA, QNH, QWG, AWA, QWA, EQK, SHS, QFA, SFA, TFA, AYG, EKQ, ERN, EMR, EFR, AYM, EAR, EMK, EYK, EWK, QPI, QPF, NPL, SPI, SPL, NPI, SIV, SLL, SSA, AEM, TML, TLL, SML, TYL, QDM, NSN, AQL, NAT, SQQ, QTI, NAH, SQL, STH, NTT, AEK, ANV, QGL, AGL, QMV, STM, NOM, STI, TQL, SMM, SKE, SQT, SYL, ASL, SLV, NAM, TTV, TPI, TPL, ATL, DNK, DSR, ANL, SHL, SNL, TNL, QNI, QAL, ANI, QNM, QYM, SQI, SNI, QNV, SHV, SHM, QMM, ANM, AHM, SQM, NAL, AQT, EKL, NNM, TNM, SNM, QQI, TQV, TQI, THM, SQV, AAV, THL, AQI, NNV, TNN, TNI, SNN, TQM, NAI, TNV, SWT, NMV, NQI, NQL, NMI, NSS, SHF, QNF, SWL, AQM, SYV, TFM, SYM, TYM, ATI, TTI, SLI, ALI, AYI, QLV, TMI, SMI, QFI, NMY, NAY, NMN, NMF, ANF, NWT, QWM, TFT, SWM, TWM, QNW, STF, AFM, NSY, SSY, SNY, THY, TNY, QNY, THE, NSV, ATY, SKD, TNF, TWT, QWT, SFS, TFS, DRQ, NYN, DER, EKG, ENN, AWN, QWQ, TQH, ALV, QHQ, DVR, STS, SQS, AAA, QGQ, QSQ, QAQ, SIH, APY, QGN, AGQ, ASQ, AAQ, SQN, NON, ERM, ALH, NYH, TGH, ATH, QTH, TQT, QQH, AQF, QMH, EKN, ERV, ERS, AFT, EKF, EKT, QSA, NFT, QYH, NYS, TSA, QYY, AYY, AHY, QFN, AWQ, NWQ, QAW, NMS, NWN, QMF, AMH, TSN, TAH, TMH, QFH, NWS, SWN, QRD, QKD, AKE, TSS, AYH, TSH, AHQ, ADK, ADR, ASA, ASS, SNS, QSS, SAH, SMH, QAH, AAH, AHN, QNS, QAS, ANS, ANN, SWA, SWS, TWS, ERI, EKS, QWS, AYS, AFS, SYS, QFS, QYN, QFT, AYT, TNS, TYS, SFT, QNQ, QYS, SYT, ANQ, SWV, SNQ, ANY, TNQ, ATF, SSQ, SSN, TWA, SGQ, SSS, TWY, SMY, TGN, TGY, QWY, SQF, TGF, QTY, TTY, AMY, QMY, ERY, TGQ, SGY, TMF, ALN, ARD, ATN, NAS, SAS, AQN, SYG, SYN, TFN, TMN, AYN, SGN, QNN, DKG, AAN, SMN, SAN, TTF, TQF, SFN, QAF, QFV, AAF, ASF, SAF, SMF, ASY, AFN, TAF, TSY, TAY, QMQ, SMQ, TYV, TGL, QAY, AAY, ADH, EDK, ASN, QYQ, SFQ, TYQ, TWQ, SWQ, AYQ, SYQ, AMV, TMV, TAV, AQV, TYT, TAT, QGI, QQV, SAQ, NAV, SAV, ASV, QSV, QAV, QAI, QAM, TAM, DTR, DOR, DMR, NNT, NYT, NMT, NST, ATT, SMV, SNT, QNT, AAT, SAM, SAL, QGV, QST, QYT, ASM, TSM, AMT, TST, ANH, SNV, TNT, ANT, SST, AST, TAI, DAR, SSM, TAL, NSM, TSL, SNF, TSI, TSV, TDK, SAT, SAI, QSL, QSI, ASI, SSL, SSV, SSI, AGI, QGM, TGM, SGM, SGL, SGV, ARL, NWE, SFL, TPY, DRP, DTK, DMK, SEY, SME, EAI, AHD, ADN, EFQ, EMH, SGE, DNQ, DNI, QLY, DGT, EGN, DYN, EWV, ESM, EAL, ASD, DKA, ADF, ADY, SLY, AEY, ETY, EAY, TGA, AVM, TPT, SVI, QSH, TTQ, QTN, TLH, AWY, SLT, AWV, QQY, NFV, SFY, TYY, SHA, TEK, QFK, NNY, EPR_NIT, NHI, NTY, SHY, NFM, NFN, NDK, NER, SER, NDR, QDK, SDR, QNA, TNA, THT, SNH, AYA, NFA, AFA, AHA, QHA, EYR, ENR, EHK, QVL, SVL, NIV, NVI, ADM, SAA, SQH, NQV, ALL, ATM, QYA, STT, DGR, NHT, SHT, QHT, TTL, NHM, QHL, DNR, QHM, QFM, QMI, AHV, THI, AHT, TNH, NHV, THV, SHI, ATV, TWI, NYV, TYI, DYR, SFI, NOF, NWM, NSL, NSP, TQY, DRS, ASP, TKD, QWI, QWN, TWN, QWV, THN, TTM, TQQ, NMQ, TYA, STQ, STN, AGN, EKH, ERT, TFH, SFH, AFH, ENK, NYQ, SFM, QGS, AHF, QLT, QHY, AQY, TRD, ERH, QWH, TYH, TRE, TAQ, AER, TDR, ASH, EKI, AFV, TYN, ALT, NGN, SYY, EWN, NGM, NAN, TAS, TFV, SFV, QAN, SGT, SAY, AFQ, TFQ, QFQ, EFK, SYH, QHV, AMM, THQ, AAI, AAM, NSQ, QAT, TSQ, SHQ, QSM, AGV, AGT, TGT, TGI, AVI, TGV, SGI, ESP, SDA, TPN, EAW, NEQ, DSN, DIK, DNA, QES, SLQ, EKD, SDH, SSF, TET, QDI, DSA, DNM, DKT, DGM, SQY, TLT, QEH, DKQ, TGE, DNS, SDV, SNE, EGT, EGV, AAP, AHG, TWK, TQA, TVT, APQ, SVT, NGT, SSH, NLV, AGY, QFF, QYF, NWV, EKA, EKY, SMG, NAG, ASG, TFI, ELK, SEK, QTL, NML, DHS, ETR, NQQ, QWL, ANP, AHH, NEK, NNN, SNA, EWR, ESR, SHN, SFG, SYA, APV, QPV, SPT, TPQ, QVI, TPV, AVL, SEM, NDM, API, QLM, QTM, QQL, QHI, NFL, AYV, NKE, TIM, QYV, SWI, DRN, AWI, SMA, NRE, QTS, QVV, QQT, QMA, QQS, QAA, ATQ, ERL, TGS, QQQ, AQQ, QHF, TAN, QSY, QSN, NGQ, TWH, TMS, QMS, TQN, TMT, QTV, THS, TTT, SMT, QMT, SMR, ADA, AAE, EPY, EYL, NWI, TSP, DGQ, NDH, QFD, EHL, ERD, DAF, ENI, ENH, QQW, AGM, ENL, EAF, EYV, ENT, SSE, AGE, QEY, DRH, NIQ, SVV, SVM, QPT, TVL, QTQ, SVQ, TIQ, NLQ, SIQ, AWL, EHH, NGY, NHF, NSA, NAA, NTH, QLI, NWH, QRE, AML, EVR, SNP, NHS, THH, NOS, TTS, AHS, EKV, NFQ, APL, QPM, NPV, SIL, TII, STV, QEL, TFL, SIT, NAQ, TMM, AAL, QQM, AMI, QFL, AHI, SYI, SSP, NVV, TTN, TAA, TQS, TMA, ALA, QQN, NSH, TTH, SRE, ERQ, QHS, SWH, SEV, AVT, ALQ, DKH, AMQ, ARE, TWV, NGL, NGV, TMQ, TLV, EER and QTT.

In some embodiments, X₁X₂X₃ is selected from the group consisting of: DMK, ATD, EEK, QMD, EFS, ERD, DDR, TDM, SAE, EHS, ENH, SWE, SNE, NNG, QAG, ERG, QSG, QNG, ASG, QFG, AMF, ELR, NEM, NNS, NNI, SDR, EQR, EHR, EWR, EQK, ESR, EKQ, EYR, ENR, EMK, EYK, EHK, EWK, QNI, TNI, TYI, SNY, DRQ, AWI, QWI, DFR, EKG, QYG, QWQ, EKN, EKF, EKT, AFH, ENK, NYS, DKH, AAG, QMF, QFH, QKD, ARE, AHQ, ADK, ADR, AHN, QNS, ANN, SWS, EKS, AFS, QFS, TNQ, TGY, NGL, ARD, AKD, AAF, SMF, AFN, SGT, TGL, ASN, SFQ, AFQ, TFQ, QFQ, EFK, SWQ, AYQ, SYQ, DQR, DMR, ASM, ANT, SHQ, TSL, SNF, QSL, ASI, SGL, SGI, NDA, DHQ, DFK, DTK, DNA, SDS, TDH, DGL, QDV, SDM, DKT, DGM, SQY, DKS, SSD, EMV, TSE, EYV, SSE, EGM, SDF, TTG, QLG, NNP, AGS, QSF, TLM, NGA, AWY, NKD, SWY, TFF, NSG, NGY, QEK, SEK, TEK, AQA, NVL, NHI, TER, NML, NMA, NNA, NHN, ANP, AMS, NER, SER, DKK, NMM, QDK, AHS, TNA, THT, SNH, QNH, ESK, SHS, AYM, SSA, NAT, QLM, QQM, SQL, SNI, QNV, SHV, SHM, NNV, TNN, AHT, TNH, NHV, TQM, THV, SHI, NYL, NMV, NFL, AYV, NOI, NQL, NMI, NSS, SHF, SYM, NYV, AYI, SWI, QFI, ANF, TFT, TWM, QNW, NSY, THY, TNY, NYN, AWN, SMA, NSH, TGH, SFH, SWH, SFM, NFT, QYH, QGS, TGS, TSA, QQQ, QYY, AMQ, TAN, AWH, NMS, TSN, TAH, TMH, QWH, NWS, SWN, QRD, TYH, AKE, TAQ, AER, AYH, ASA, SNS, QSS, SAH, SMH, ASH, QAS, ANS, SYS, QYN, TYN, TNS, SFT, ANQ, SGS, SGH, SSS, TWY, QTY, SYY, NGM, TMS, SYG, TON, SYN, TFN, TMN, AYN, SGN, QNN, SMN, SAN, QAF, ASF, ASY, TAS, TAF, TFV, SMQ, SFV, TMT, QAN, SAY, EDK, AMV, QHV, THS, TAT, QGI, QQV, NAV, SAV, ASV, QSV, QAV, AAI, AAM, QAM, TAM, DTR, NNT, NYT, NMT, ATT, QTT, TTT, QAT, SMV, SNT, QNT, AAT, SAM, SAL, QGV, QST, QYT, TSM, ANH, SNV, TSQ, TNT, SST, AST, TAI, TSI, TSV, TDK, SAT, QSM, SAI, QSI, SSV, SSI, AGT, TGT, AGI, QGM, TGM, TGV, SGM, SGV, SQE, EHT, EYL, DSG, SFE, QND, TQA, AWV, NFV, TSG, SSG, DRG, ANW, TFI, TSF, QNL, STL, TWL, NNF, NND, NYM, NFI, ETR, SNP, EIR, NFS, NNH, NNL, AHL, NNQ, NDR, NNN, QDR, SDK, SNA, AFG, NYA, QHA, QFA, SHN, NFQ, SYA, TFA, EMR, EFR, EAR, NOM, TTL, TQL, SMM, SKE, SYL, ASL, SLV, TTV, AAL, NHL, NHM, QYL, QHL, ATL, DNR, DSR, ANL, SHL, SNL, TNL, QAL, AMI, ANI, QNM, QYM, QHM, QFM, QMM, QMI, ANM, AHM, SQM, AQT, EKL, NNM, TNM, SNM, QQI, TQV, TQI, THI, QHI, THL, SNN, TNV, SWL, AQM, SYV, TFM, TYM, TWI, AFI, ATI, DYR, QLV, TMI, SYI, SFI, NWM, NWT, NSL, STF, SNW, AFM, SSY, NSV, TNF, NAF, TFS, QWV, ERT, TQT, AFT, NYQ, ERL, NRD, TRD, AWQ, TDR, TSH, ASS, AYT, TYS, QYS, SYT, SWV, SNQ, SMS, NAS, SAS, QFV, SAF, TWQ, TAV, AMM, TYT, QAI, TST, DAR, SSM, TAL, NES, DWK, DLR, DIR, SDT, EAN, ETH, TYD, SYE, EAM, TND, AYD, TSP, DRT, NDH, SSF, EFH, DNQ, DSH, DNT, SDI, DKQ, EWT, EAL, EAS, ESL, ASD, EAH, ASE, EAF, ENT, SIY, QDY, APG, AIG, SSW, NPG, ERP, TVI, QSH, SLS, EDR, AWL, SLH, EQH, EHH, NWV, EKY, STY, DRI, EYH, DRV, SPY, NNY, DWR, ERA, QRE, EVR, SRD, AWA, SFA, QTI, SHT, THM, AQI, AHI, SWT, ATV, TTI, SMI, QYV, NMY, SWM, QNY, TWT, QWT, TQY, NRE, QTS, QHQ, DVR, AAA, TYA, EKH, ALH, AWT, ATH, SRE, TFH, QMH, ERV, ERS, QHY, ALQ, QSY, ERH, TRE, TSS, QAH, AAH, SWA, TWS, QWS, AYS, AFV, QNQ, ANY, ALT, SMY, TWV, TTY, AMY, QMY, ERY, TWH, TSY, TAY, TYV, QAY, AAY, QYQ, TYQ, SYH, AQV, EER, THQ, and SMT.

In some embodiments, RGDX₇X₈X₉X₁₀ has an amino acid sequence selected from SEQ ID NOs.: 238-44858.

In some embodiments, RGDX₇X₈X₉X₁₀ has an amino acid sequence selected from SEQ ID NOs.: 238-248.

In some embodiments, RGDX₇X₈X₉X₁₀ has an amino acid sequence selected from SEQ ID NOs.: 238-338.

In some embodiments, RGDX₇X₈X₉X₁₀ has an amino acid sequence selected from SEQ ID NOs.: 238-438.

In some embodiments, RGDX₇X₈X₉X₁₀ has an amino acid sequence selected from SEQ ID NOs.: 238-538.

In some embodiments, RGDX₇X₈X₉X₁₀ has an amino acid sequence selected from SEQ ID NOs.: 238-638.

In some embodiments, RGDX₇X₈X₉X₁₀ has an amino acid sequence selected from SEQ ID NOs.: 238-738.

In some embodiments, RGDX₇X₈X₉X₁₀ has an amino acid sequence selected from SEQ ID NOs.: 3881, 12092, 14601, 15342, 21498, and 31396.

In some embodiments, RGDX₇X₈X₉X₁₀ has the amino acid sequence of SEQ ID NO: 238.

In some embodiments, X₁X₂X₃RGDX₇X₈X₉X₁₀ has an amino acid sequence selected from SEQ ID NOs.: 44859-44883, 44911, 44912, 44913, 44918-44919, and 48391-157057.

In some embodiments, X₁X₂X₃RGDX₇X₈X₉X₁₀ has an amino acid sequence selected from SEQ ID NOs.: 44864-44867, and 44879-44883.

In some embodiments, X₁X₂X₃RGDX₇X₈X₉X₁₀ has an amino acid sequence selected from SEQ ID NOs.: 44859-44878, 44911, 44912, 44913, 44918-44919, and 48391-157057.

In some embodiments, the X₇ is selected from R, F, H, L, Q, R, and Y.

In some embodiments, X₇ is R. In some embodiments, X₇ is Y or H. In some embodiments, X> is F or Y.

In some embodiments, the X₇ is selected from S, G, D, I, L, N, Q, T, and V.

In some embodiments, X₈ is S or G. In some embodiments, X₈ is T, G or S. In some embodiments, X₈ is N or Q.

In some embodiments, the X₉ is selected from any of the amino acids. In some embodiments, X₉ is selected from V, S, N, G, Q, L, T, and Y.

In some embodiments, X₉ is selected from V, S, N, G, Q, L, T, and Y. In some embodiments, X₉ is selected from V or Q. In some embodiments, X₉ is selected from S or V. In some embodiments, X₉ is selected from N or S.

In some embodiments, X₁₀ is selected from I, V, S, L, M, R, T, and Q.

In some embodiments, X₁₀ is I or V. In some embodiments, X₁₀ is V, L or M. In some embodiments, X₁₀ is T or L.

In some embodiments, the targeting peptide has a sequence of X₁X₂X₃RGDRGVV (SEQ ID NO: 98928), X₁X₂X₃RGDRSVV (SEQ ID NO: 98931), X₁X₂X₃RGDRGQI (SEQ ID NO: 98927), X₁X₂X₃RGDRSQT (SEQ ID NO: 98930), X₁X₂X₃RGDRQGI (SEQ ID NO: 98929), X₁X₂X₃RGDFQNT (SEQ ID NO: 98934), X₁X₂X₃RGDHGVL (SEQ ID NO: 98938), X₁X₂X₃RGDYTSV (SEQ ID NO: 98941), X₁X₂X₃RGDYTSM (SEQ ID NO: 98942), X₁X₂X₃RGDLTVT (SEQ ID NO: 98935), X₁X₂X₃RGDFNNT (SEQ ID NO: 98943), X₁X₂X₃RGDYSSV (SEQ ID NO: 98937), X₁X₂X₃RGDHVNL (SEQ ID NO: 98924), X₁X₂X₃RGDQSTL (SEQ ID NO: 98926), X₁X₂X₃RGDLIGR (SEQ ID NO: 98925), X₁X₂X₃RGDFNNL (SEQ ID NO: 98933), X₁X₂X₃RGDLLLS (SEQ ID NO: 98932), X₁X₂X₃RGDYNSL (SEQ ID NO: 98940), X₁X₂X₃RGDRDYL (SEQ ID NO: 98939), or X₁X₂X₃RGDYVGL (SEQ ID NO: 98936).

In some embodiments, the targeting peptide has a sequence of X₁X₂X₃RGDRGVV (SEQ ID NO: 98928), X₁X₂X₃RGDRSVV (SEQ ID NO. 98931) or X₁X₂X₃RGDRGQI (SEQ ID NO: 98927).

In some embodiments, the targeting peptide has a sequence of X₁X₂X₃RGDYTSV (SEQ ID NO: 98941), X₁X₂X₃RGDYTSM (SEQ ID NO: 98942), X₁X₂X₃RGDRGVV (SEQ ID NO: 98928), X₁X₂X₃RGDRSVV (SEQ ID NO: 98931), X₁X₂X₃RGDYSSV (SEQ ID NO: 98937), or X₁X₂X₃RGDHGVL (SEQ ID NO: 98938).

In some embodiments, the targeting peptide has a sequence of X₁X₂X₃RGDFQNT (SEQ ID NO: 98934), X₁X₂X₃RGDHGVL (SEQ ID NO: 98938), X₁X₂X₃RGDLIGR (SEQ ID NO: 98925), X₁X₂X₃RGDRGQI (SEQ ID NO. 98927), X₁X₂X₃RGDRGVV (SEQ ID NO: 98928), X₁X₂X₃RGDYTSM (SEQ ID NO: 98942) or X₁X₂X₃RGDYTSV (SEQ ID NO: 98941).

In some embodiments, X₁X₂X₃ is selected from the group consisting of EFK, AAY, DQK, QVY, DKL, DNV, ENF, EWK, QNV, and TFM.

In some embodiments, the targeting peptide has an amino acid sequence selected from: RGDRSX₉I, RGDRGX₉I, RGDRSX₉V, or RGDRGX₉V.

In some embodiments, the targeting peptide has an amino acid sequence selected from X₁X₂X₃RGDRGQI (SEQ ID NO: 98927), X₁X₂X₃RGDRSVV (SEQ ID NO: 98931) or X₁X₂X₃RGDRGVV (SEQ ID NO: 98928).

In some embodiments, X₁ is Dor E, X₂ is K, E, D, A, S, F or N, and X₃ is Y, V, or F. In some embodiments, X₁X₂X₃ is selected from the group consisting of: DII, DWM, EEI, DML, DWI, SLE, EIN, NHE, DFI, EEL, TEQ, TDA, EDT, NEV, TDW, QFE, EDY, DTT, EPL, SEN, SEQ, TAE, EVN, ELN, DVQ, ETI, EVI, ESV, ETW, SEW, DNW, EVF, EAW, EPF, EIY, EIF, EPY, DVI, DMM, DQI, DHL, DTL, DVL, NDL, DLL, DMQ, NEF, DFL, DIM, TEW, DYI, SDY, DYY, DHF, DKE, DTW, DTI, ELY, TEY, TEI, DAI, DQY, DMY, EWG, DMV, DMI, EPH, QEG, DIN, NEI, EYY, DIV, SEG, DVG, DYQ, EGF, NDI, EGY, DVF, DVH, DGF, DIY, DSF, DGW, EHY, DRE, TEH, DTS, NEN, NEM, NEH, TEN, DSN, DVT, DQS, DKD, DTH, DVV, DQK, NET, DKP, TEV, NDS, QET, EVL, SET, SDT, AEQ, QEF, SEY, SEF, SPF, EGQ, ETH, TDQ, QEA, QDQ, AEN, ESS, NDT, EFM, EFI, EHM, DFM, QDT, SME, DYT, EHV, ENV, EAV, EAI, ESI, DAT, ENQ, EAM, ADN, EFQ, SDS, TDH, SDH, DAS, TWE, SSF, DRD, EFL, TDF, QDA, EMH, SGE, AEW, DAH, TET, TDM, TNE, SAE, NSE, SFE, QDI, DSA, ADV, SEI, AEI, QDV, ADT, DNM, DNQ, ADL, TDL, SDL, SDM, TDV, DNI, DHY, DTY, DAA, DSY, QLY, DVM, DAY, DMT, DQT, DAQ, DTV, DSH, QDF, DST, DNL, DSI, DFV, DNY, DAF, DKI, DKF, DTM, DSL, NDV, TDI, DSV, DAV, DKV, DAM, DNV, DKM, DKL, DKW, DSM, ENI, SDI, DKT, QGE, NNE, QYW, AGM, ESH, QDH, QEH, DYH, ENS, DKQ, TGE, DSS, EST, DSQ, DNS, EFV, SWE, DKN, DKS, SEH, EAL, EAS, ADQ, SDQ, ADI, SDV, EAH, ASE, EAF, EYV, DKA, SNE, AGE, EGV, EQF, ETF, EVY, EQY, QEY, QEN, SAD, AEF, EMF, SDF, ADF, ADY, AVF, TLY, SIY, SLY, AIY, DYV, AEY, ENF, EMY, QDY, ETY, EAY, TVY, SVY, QVF, QVY, TMY, DKY, ALY, AVY, TMW, EQW, SPW, SIW, DTQ, TFF, AMF, EYT, DRV, SPY, SIF, SLF, ENY, EWY, EYI, EFY, TLF, TVF, TYY, TFY, SVF, SYW, TAW, SAW, TIY, NIM, NFM, NII, NIV, SII, SIV, SEM, TTI, NSY, SSY, SNY, SEV, AEH, ALT, SGQ, QTY, SYY, AMY, QMY, SGF, SGY, ALF, TTF, SEN, QAF, ASF, SAF, TAF, TAY, TGL, SAY, QAY, ADH, ASI, SSV, SSI, TGV, SGV, DPH, TEG, DLY, ESE, TEF, NPE, DHA, DLI, DWG, EWM, EET, EIL, TEL, DYL, EMW, EVW, EPV, EDV, SLD, EGH, QRY, TDG, SQE, ELH, EYW, EMM, EFF, QEI, EQG, DVS, ESP, EIQ, EPM, EPQ, SPE, EPN, NQD, ELQ, TDT, ELT, EPI, ETM, ETG, EHI, EMI, EIT, ETV, EVV, EVM, TPY, QEW, EIW, EPT, EIH, DFY, DQL, NDA, DIW, TDY, ELF, DVY, ESG, EPG, EIG, ELG, EVG, DHI, EIS, DYM, DFT, DFH, EQS, TPF, EEF, DWN, SDN, EYL, AET, DMN, QSP, DNA, DHM, DHT, EQI, ESN, EYF, EHF, NAD, AEG, AES, TEM, SEL, DYS, EVH, EAN, EGL, TFE, ETL, TYE, SHE, ELM, TYD, SYE, ESA, EFT, EAT, AMD, AND, DMS, QMD, SLQ, EYS, QDN, EYQ, EKD, QYD, QFD, DER, AFD, EAA, EFS, DHV, QSE, TSD, SFD, DGY, TWD, QWD, EHL, QGD, QSD, AWD, AGD, QIY, EMQ, EAG, ERD, DDR, DAL, QDL, DNT, EGN, ENH, NGE, DYN, DNH, DNF, ESQ, ESM, ESL, SSD, ENL, EMV, QDS, TSE, SWD, ASD, ENT, SSE, EGM, EGS, EGT, EGI, AIF, NIN, NIQ, NYW, EPW, EGG, TIW, QIW, TIW, EKW, NLY, SVM, TPT, AAW, AIH, TIH, QGT, QFF, QYF, QFY, EHH, QLH, TYF, STY, DRL, EYM, SFF, QLF, QIF, APF, TYW, QII, QPF, SPI, TIL, SVL, NVI, SIM, SLL, QEL, AEM, AEL, QEV, QDM, NDM, SYL, ASL, TPL, ANI, NMI, TIM, AEV, TMI, SWI, SFI, SNW, TNF, NAF, ASP, QSQ, APY, SFM, TAN, QAW, SGS, SGH, TGF, QSN, ERY, QMN, TGQ, NGM, NGV, AMN, ALN, QFV, AAF, TFV, TSY, EDK, ASN, SAV, QSV, QGV, QST, ASM, TAI, QSM, QSL, QSI, SSL, AGV, TGT, AGI, QGM, TGM, SGM, SGI, THD, QVE, ALE, QLE, EWP, AFP, AEE, ADE, NIE, TYP, QIE, TFP, SYP, ELW, DVW, NEW, EPA, EVA, TLE, AWE, NME, QME, ATE, AYE, DQM, TPD, DFG, QPE, EDI, EWL, ELL, QPD, EWF, ALD, SVD, QVD, ADA, APD, AHE, EQL, DTA, AFE, DAW, QAE, TME, STE, THE, QID, AAE, EIA, EML, SDA, SIE, TQE, TVD, NWE, NOE, EYG, QYP, EIV, EII, QFP, TID, EQN, EVS, TYG, EAQ, ELV, QLD, TLD, QQD, TIN, TLN, SPN, TPN, NFD, NEY, DSW, NMD, DGA, DYA, DIH, DNP, DQW, DQV, EFA, EPS, DEN, DWV, ELI, DTF, EQT, DHW, ESF, DEK, DMH, EWS, EHA, TTD, EWI, QLQ, SQD, QHD, NSD, EMA, TND, ATD, DGS, TAD, TMD, QES, AHD, QAD, DGH, DGL, NTE, ENG, SMD, NDQ, EMS, ENA, EMT, EYN, DDK, EWA, EWQ, EFH, QWE, DGT, DGM, EWV, EWH, EWT, QND, DGV, ESY, AAD, SND, ANE, TIG, QIG, AVD, SFP, AWP, QDW, SLW, TPA, AII, TIV, SWP, AFY, APN, AVN, TVN, TIS, TIQ, TLQ, AIQ, QVN, NFY, QFG, EMN, NVF, SYF, SFW, TPQ, AIL, TWI, QWN, TWN, QWQ, AQF, ASA, TWV, EWN, TMF, NGI, ATN, SAS, TMQ, TMN, AYN, TQF, SMF, QMQ, SMQ, QAN, AAY, SFQ, TYQ, TFQ, TWQ, AVI, and SGL.

In some embodiments, X₁X₂X₃ is selected from the group consisting of: DAV, DKW. EAY, AEY, DFV, DKF, DKI, DKL, DNV, DNY, DSL, DSV, EFI, SEF, SEY, SLY, ADF, ADY, ALY, AVF, DAF, DAL, DAM, DAT, DHV, DIV, DKA, DKM, DKT, DKV, DKY, DMI, DNF, DNI, DQT, DSL, DVY, DYN, DYV, EAT, EAW, EFV, EGL, EIY, EMF, EMY, ENF, EPF, EPY, EQY, ESY, ETF, EWI, EWT, EYI, EYV, NEM, QDF, QDY, QEY, QLY, QND, QVF, QVY, SDL, SDV, SEH, SII, SIY, SSL, SVY, SWD, SWE, TDF, TDV, TDY, TEF, TEY, TIY, TLY, and TWQ.

In some embodiments, X₇ is selected from Y and H; X₈ is selected from T, G and S; X₉ is selected from S and V; X₁₀ is selected from V, L and M.

In some embodiments, the targeting peptide has an amino acid sequence selected from: X₁X₂X₃RGDHGVL (SEQ ID NO: 98938), X₁X₂X₃RGDYSSV (SEQ ID NO: 98937). X₁X₂X₃RGDYTSM (SEQ ID NO: 98942) or X₁X₂X₃RGDYTSV (SEQ ID NO: 98941).

In some embodiments, X₁ is S, A or T. X₂ is N, A or Y, and X₃ is I, V, M, Q, T, Y or K.

In some embodiments, X₁X₂X₃ is selected from the group consisting of: APW, TEL, TDA, QPY, SPN, EHY, DWK, DLK, DFK, DVK, NSI, DIR, SPF, SEL, DRT, DRF, ADL, TDL, SDL, DNY, DKI, NDV, DKM, DNH, DNF, DSS, EST, EWT, DKN, DKS, SEH, ESQ, ESL, QND, EAH, AIF, AVF, QVF, TMY, ALY, NNG, NIF, NTF, NFF, AWF, NPY, SWF, AII, AYF, AQW, NFY, AGP, QQF, TKE, TNG, NSF, NAW, QAG, ERG, NKD, QSG, QNG, EAK, QWF, SWY, TFF, TYF, NYY, QFG, NWA, AMF, STY, TNW, ANW, AWM, TSF, DRL, DRV, SPY, NVF, SIF, QLF, SLF, QIF, SYF, APF, AGF, SVF, SAW, TIY, DHK, DAK, EGK, DYK, QNL, QPL, SPV, NPM, STL, NIL, AGA, NTV, SQA, QQA, NNF, NWL, NMA, NNA, NHN, NFH, ELR, NYM, ERA, NFS, SRD, NMM, NNL, NNQ, NNS, QDR, SDK, NNI, AQH, ANA, QNH, QWG, AWA, QWA, EQK, SHS, QFA, SFA, TFA, AYG, EKQ, ERN, EMR, EFR, AYM, EAR, EMK, EYK, EWK, QPI, QPF, NPL, SPI, SPL, NPI, SIV, SLL, SSA, AEM, TML, TLL, SML, TYL, QDM, NSN, AQL, NAT, SQQ, QTI, NAH, SQL, STH, NTT, AEK, ANV, QGL, AGL, QMV, STM, NOM, STI, TQL, SMM, SKE, SQT, SYL, ASL, SLV, NAM, TTV, TPI, TPL, ATL, DNK, DSR, ANL, SHL, SNL, TNL, QNI, QAL, ANI, QNM, QYM, SQL SNI, QNV, SHV, SHM, QMM, ANM, AHM, SQM, NAL, AQT, EKL, NNM, TNM, SNM, QQI, TQV, TQI, THM, SQV, AAV, THL, AQI NNV, TNN, TNI, SNN, TQM, NAI, INV, SWT, NMV, NQI, NQL, NMI, NSS, SHF, QNF, SWL, AQM, SYV, TFM, SYM, TYM, ATI, TTI, SLI, ALI, AYI, QLV, TMI, SMI, QFI, NMY, NAY, NMN, NMF, ANF, NWT, QWM, TFT, SWM, TWM, QNW, STF, AFM, NSY, SSY, SNY, THY, TNY, QNY, THE, NSV, ATY, SKD, TNF, TWT, QWT, SFS, TFS, DRQ, NYN, DFR, EKG, ENN, AWN, QWQ, TQH, ALV, QHQ, DVR, STS, SQS, AAA, QGQ, QSQ, QAQ, SIH, APY, QGN, AGQ, ASQ, AAQ, SQN, NON, ERM, ALH, NYH, TGH, ATH, QTH, TQT, QQH, AQF, QMH, EKN, ERV, ERS, AFT, EKF, EKT, QSA, NFT, QYH, NYS, TSA, QYY, AYY, AHY, QFN, AWQ, NWQ, QAW, NMS, NWN, QMF, AMH, TSN, TAH, TMH, QFH, NWS, SWN, QRD, QKD, AKE, TSS, AYH, TSH, AHQ, ADK, ADR, ASA, ASS, SNS, QSS, SAH, SMH, QAH, AAH, AHN, QNS, QAS, ANS, ANN, SWA, SWS, TWS, ERI, EKS, QWS, AYS, AFS, SYS, QFS, QYN, QFT, AYT, TNS, TYS, SFT, QNQ, QYS, SYT, ANQ, SWV, SNQ, ANY, TNQ, ATF, SSQ, SSN, TWA, SGQ, SSS, TWY, SMY, TGN, TGY, QWY, SQF, TGF, QTY, TTY, AMY, QMY, ERY, TGQ, SGY, TMF, ALN, ARD, ATN, NAS, SAS, AQN, SYG, SYN, TEN, TMN, AYN, SGN, QNN, DKG, AAN, SMN, SAN, TTF, TQF, SFN, QAF, QFV, AAF, ASF, SAF, SMF, ASY, AFN, TAF, TSY, TAY, QMQ, SMQ, TYV, TGL, QAY, AAY, ADH, EDK, ASN, QYQ, SFQ, TYQ, TWQ, SWQ, AYQ, SYQ, AMV, TMV, TAV, AQV, TYT, TAT, QGI, QQV, SAQ, NAV, SAV, ASV, QSV, QAV, QAI, QAM, TAM, DTR, DOR, DMR, NNT, NYT, NMT, NST, ATT, SMV, SNT, QNT, AAT, SAM, SAL, QGV, QST, QYT, ASM, TSM, AMT, TST, ANH, SNV, TNT, ANT, SST, AST, TAI, DAR, SSM, TAL, NSM, TSL, SNF, TSI, TSV, TDK, SAT, SAL, QSL, QSI, ASI, SSL, SSV, SSI, AGI, QGM, TGM, SGM, SGL, SGV, ARL, NWE, SFL, TPY, DRP, DTK, DMK, SEY, SME, EAI, AHD, ADN, EFQ, EMH, SGE, DNQ, DNI, QLY, DGT, EGN, DYN, EWV, ESM, EAL, ASD, DKA, ADF, ADY, SLY, AEY, ETY, EAY, TGA, AVM, TPT, SVI, QSH, TTQ, QTN, TLH, AWY, SLT, AWV, QQY, NFV, SFY, TYY, SHA, TEK, QFK, NNY, EPR, NIT, NHI, NTY, SHY, NFM, NFN, NDK, NER, SER, NDR, QDK, SDR, QNA, TNA, THT, SNH, AYA, NFA, AFA, AHA, QHA, EYR, ENR, EHK, QVL, SVL, NIV, NVI, ADM, SAA, SQH, NQV, ALL, ATM, QYA, STT, DGR, NHT, SHT, QHT, TTL, NHM, QHL, DNR, QHM, QFM, QMI, AHV, THI, AHT, TNH, NHV, THV, SHI, ATV, TWI, NYV, TYI, DYR, SFI, NQF, NWM, NSL, NSP, TQY, DRS, ASP, TKD, QWI, QWN, TWN, QWV, THN, TTM, TQQ, NMQ, TYA, STQ, STN, AGN, EKH, ERT, TFH, SFH, AFH, ENK, NYQ, SFM, QGS, AHF, QLT, QHY, AQY, TRD, ERH, QWH, TYH, TRE, TAQ, AER, TDR, ASH, EKI, AFV, TYN, ALT, NGN, SYY, EWN, NGM, NAN, TAS, TFV, SFV, QAN, SGT, SAY, AFQ, TFQ, QFQ, EFK, SYH, QHV, AMM, THQ, AAI, AAM, NSQ, QAT, TSQ, SHQ, QSM, AGV, AGT, TGT, TGI, AVI, TGV, SGI, ESP, SDA, TPN, EAW, NEQ, DSN, DIK, DNA, QES, SLQ, EKD, SDH, SSF, TET, QDI, DSA, DNM, DKT, DGM, SQY, TLT, QEH, DKQ, TGE, DNS, SDV, SNE, EGT, EGV, AAP, AHG, TWK, TQA, TVT, APQ, SVT, NGT, SSH, NLV, AGY, QFF, QYF, NWV, EKA, EKY, SMG, NAG, ASG, TFI, ELK, SEK, QTL, NML, DHS, ETR, NQQ, QWL, ANP, AHH, NEK, NNN, SNA, EWR, ESR, SHN, SFG, SYA, APV, QPV, SPT, TPQ, QVI, TPV, AVL, SEM, NDM, API, QLM, QTM, QQL, QHI, NFL, AYV, NKE, TIM, QYV, SWI, DRN, AWI, SMA, NRE, QTS, QVV, QQT, QMA, QQS, QAA, ATQ, ERL, TGS, QQQ, AQQ, QHF, TAN, QSY, QSN, NGQ, TWH, TMS, QMS, TON, TMT, QTV, THS, TTT, SMT, QMT, SMR, ADA, AAE, EPY, EYL, NWI, TSP, DGQ, NDH, QFD, EHL, ERD, DAF, ENI, ENH, QQW, AGM, ENL, EAF, EYV, ENT, SSE, AGE, QEY, DRH, NIQ, SVV, SVM, QPT, TVL, QTQ, SVQ, TIQ, NLQ, SIQ, AWL, EHH, NGY, NHF, NSA, NAA, NTH, QLI, NWH, QRE, AML, EVR, SNP, NHS, THH, NOS, TTS, AHS, EKV, NFQ, APL, QPM, NPV, SIL, TII, STV, QEL, TFL, SIT, NAQ, TMM, AAL, QQM, AMI, QFL, AHI, SYL, SSP, NVV, TIN, TAA, TQS, TMA, ALA, QQN, NSH, TTH, SRE, ERQ, QHS, SWH, SEV, AVT, ALQ, DKH, AMQ, ARE, TWV, NGL, NGV, TMQ, TLV, EER and QTT.

In some embodiments, X₁X₂X₃ is selected from ANY, SNI, AAI, AAM, ANT, AST, AYQ, EHK, ENK, ENR, SFQ, SSI, TAY, TDK, TNT, AAF, AAL, AAY, ADK, AFA, ANF, ANI, ANQ, ANS, AQM, ARE, ASV, AYH, AYT, EMK, EWK, NNM, QAF, QAI, QAM, QAT, QAY, QFT, QGM, QHL, QNF, QNQ, QNS, QNT, QNV, QNY, SAH, SAI, SAL, SFT, SFV, SHI, SHV, SMM, SNF, SNM, SNN, SNQ, SNV, SNY, SQI, SQV, SSL, SWQ, SWS, SYI, SYM, SYN, SYQ, TAM, TAT, TDR, TFM, THV, TNF, TNH, TNI, TNM, TNQ, TNV, TSY, TWA, and TYM.

In some embodiments, X₇ is selected from F and Y; X₈ is selected from N and Q; X₉ is selected from N and S; X₁₀ is selected from T and L.

In some embodiments, the targeting peptide has an amino acid sequence selected from: X₁X₂X₃RGDFNNT (SEQ ID NO: 98943), X₁X₂X₃RGDENNL (SEQ ID NO: 98933), X₁X₂X₃RGDFQNT (SEQ ID NO: 98934) or X₁X₂X₃RGDYNSL (SEQ ID NO: 98940).

In some embodiments, X₁ is S, A or E, X₂ is N, Y or S, X₃ is I, Q, R, V, T, M or K.

In some embodiments, X₁X₂X₃ is selected from the group consisting of: DMK, ATD, EEK, QMD, EFS, ERD, DDR, TDM, SAE, EHS, ENH, SWE, SNE, NNG, QAG, ERG, QSG, QNG, ASG, QFG, AMF, ELR, NFM, NNS, NNI, SDR, EQR, EHR, EWR, EQK, ESR, EKQ, EYR, ENR, EMK, EYK, EHK, EWK, QNI, TNI, TYI, SNY, DRQ, AWI, QWI, DFR, EKG, QYG, QWQ, EKN, EKF, EKT, AFH, ENK, NYS, DKH, AAG, QMF, QFH, QKD, ARE, AHQ, ADK, ADR, AHN, QNS, ANN, SWS, EKS, AFS, QFS, TNQ, TGY, NGL, ARD, AKD, AAF, SMF, AFN, SGT, TGL, ASN, SFQ, AFQ, TFQ, QFQ, EFK, SWQ, AYQ, SYQ, DQR, DMR, ASM, ANT, SHQ, TSL, SNF, QSL, ASI, SGL, SGI, NDA, DHQ, DFK, DTK, DNA, SDS, TDH, DGL, QDV, SDM, DKT, DGM, SQY, DKS, SSD, EMV, TSE, EYV, SSE, EGM, SDF, TTG, QLG, NNP, AGS, QSF, TLM, NGA, AWY, NKD, SWY, TFF, NSG, NGY, QEK, SEK, TEK, AQA, NVL, NHI, TER, NML, NMA, NNA, NHN, ANP, AMS, NER, SER, DKK, NMM, QDK, AHS, TNA, THT, SNH, QNH, ESK, SHS, AYM, SSA, NAT, QLM, QQM, SQI, SNI, QNV, SHV, SHM, NNV, TNN, AHT, TNH, NHV, TQM, THV, SHI, NYL, NMV, NFL, AYV, NQI, NQL, NMI, NSS, SHF, SYM, NYV, AYI, SWI, QFI, ANF, TFT, TWM, QNW, NSY, THY, TNY, NYN, AWN, SMA, NSH, TGH, SFH, SWH, SFM, NFT, QYH, QGS, TGS, TSA, QQQ, QYY, AMQ, TAN, AWH, NMS, TSN, TAH, TMH, QWH, NWS, SWN, QRD, TYH, AKE, TAQ, AER, AYH, ASA, SNS, QSS, SAH, SMH, ASH, QAS, ANS, SYS, QYN, TYN, TNS, SFT, ANQ, SGS, SGH, SSS, TWY, QTY, SYY, NGM, TMS, SYG, TON, SYN, TFN, TMN, AYN, SGN, QNN, SMN, SAN, QAF, ASF, ASY, TAS, TAF, TFV, SMQ, SFV, TMT, QAN, SAY, EDK, AMV, QHV, THS, TAT, QGI, QQV, NAV, SAV, ASV, QSV, QAV, AAI, AAM, QAM, TAM, DTR, NNT, NYT, NMT, ATT, QTT, TTT, QAT, SMV, SNT, QNT, AAT, SAM, SAL, QGV, QST, QYT, TSM, ANH, SNV, TSQ, TNT, SST, AST, TAI, TSI, TSV, TDK, SAT, QSM, SAI, QSI, SSV, SSI, AGT, TGT, AGI, QGM, TGM, TGV, SGM, SGV, SQE, EHT, EYL, DSG, SFE, QND, TQA, AWV, NFV, TSG, SSG, DRG, ANW, TFI, TSF, QNL, STL, TWL, NNF, NND, NYM, NFI, ETR, SNP, EIR, NFS, NNH, NNL, AHL, NNQ, NDR, NNN, QDR, SDK, SNA, AFG, NYA, QHA, QFA, SHN, NFQ, SYA, TFA, EMR, EFR, EAR, NOM, TTL, TQL, SMM, SKE, SYL, ASL, SLV, TTV, AAL, NHL, NHM, QYL, QHL, ATL, DNR, DSR, ANL, SHL, SNL, TNL, QAL, AMI, ANI, QNM, QYM, QHM, QFM, QMM, QMI, ANM, AHM, SQM, AQT, EKL, NNM, TNM, SNM, QQI, TQV, TQI, THI, QHI, THL, SNN, TNV, SWL, AQM, SYV, TFM, TYM, TWI, AFI, ATI, DYR, QLV, TMI, SYI, SFI, NWM, NWT, NSL, STF, SNW, AFM, SSY, NSV, TNF, NAF, TFS, QWV, ERT, TQT, AFT, NYQ, ERL, NRD, TRD, AWQ, TDR, TSH, ASS, AYT, TYS, QYS, SYT, SWV, SNQ, SMS, NAS, SAS, QFV, SAF, TWQ, TAV, AMM, TYT, QAI, TST, DAR, SSM, TAL, NES, DWK, DLR, DIR, SDT, EAN, ETH, TYD, SYE, EAM, TND, AYD, TSP, DRT, NDH, SSF, EFH, DNQ, DSH, DNT, SDI, DKQ, EWT, EAL, EAS, ESL, ASD, EAH, ASE, EAF, ENT, SIY, QDY, APG, AIG, SSW, NPG, ERP, TVI, QSH, SLS, EDR, AWL, SLH, EQH, EHH, NWV, EKY, STY, DRI, EYH, DRV, SPY, NNY, DWR, ERA, QRE, EVR, SRD, AWA, SFA, QTI, SHT, THM, AQI, AHI, SWT, ATV, TTI, SMI, QYV, NMY, SWM, QNY, TWT, QWT, TQY, NRE, QTS, QHQ, DVR, AAA, TYA, EKH, ALH, AWT, ATH, SRE, TFH, QMH, ERV, ERS, QHY, ALQ, QSY, ERH, TRE, TSS, QAH, AAH, SWA, TWS, QWS, AYS, AFV, QNQ, ANY, ALT, SMY, TWV, TTY, AMY, QMY, ERY, TWH, TSY, TAY, TYV, QAY, AAY, QYQ, TYQ, SYH, AQV, EER, THQ, and SMT.

In some embodiments, X₁X₂X₃ is selected from the group consisting of: ADR, ASI, EFK, EHK, EWK, SYQ, AAF, AAT, AAY, AFI, AFQ, AGI, AGT, AHI, ANH, ANM, ANN, AQI, ASA, ASH, AST, ASV, AWT, AYQ, AYT, DAR, DMK, DQR, DVR, EAR, EFR, EMK, EMR, EQK, ERA, ERS, ESR, NDA, NDR, NMI, NMV, NNM, NNN, NYL, NYM, NYN, NYQ, NYV, QAM, QFQ, QFV, QGV, QNH, QNI, QNM, QQV, QSF, QSY, SAL SAM, SAS, SDR, SFQ, SGH, SGM, SGT, SGV, SHL, SHM, SHQ, SHV, SMH, SNA, SNE, SNF, SNI, SNQ, SNT, SRE, SST, SWQ, SWT, SYG, SYY, TFQ, THL, THQ, THV, TMI, TNL, TNS, TSH, TSQ, and TWQ.

In some embodiments, RGDRX₈X₉X₁₀ has an amino acid sequence selected from: RGDRGVX₁₀ (SEQ ID NO: 157058), RGDRGSX₁₀ (SEQ ID NO: 157059), RGDRGNX₁₀ (SEQ ID NO: 157060), RGDRGGX₁₀ (SEQ ID NO: 157061), RGDRGQX₁₀ (SEQ ID NO: 157062), RGDRGX₉V (SEQ ID NO: 157063), RGDRGX₉I (SEQ ID NO: 157064), RGDRGX₉S (SEQ ID NO: 157065), RGDRGX₉L (SEQ ID NO: 157066). RGDRGX₉Q (SEQ ID NO: 157067), RGDHX₈X₉L (SEQ ID NO. 157068), RGDRX₈X₉I (SEQ ID NO: 157069). RGDRX₈X₉V (SEQ ID NO. 157070). RGDRX₈X₉L (SEQ ID NO: 157071). RGDYX₈X₉L (SEQ ID NO: 157072), RGDYX₈X₉V (SEQ ID NO: 157073), RGDYX₈X₉M (SEQ ID NO: 157074), and RGDLX₈X₉T (SEQ ID NO: 157075).

In some embodiments, the modified sequence does not comprise an amino acid sequence selected from: RGDRMVF (SEQ ID NO: 157080), RGDRTVI (SEQ ID NO: 157081), SRODRPM (SEQ ID NO: 157082), ISLRGDR (SEQ ID NO: 157083), and RGDLLLS (SEQ ID NO: 1).

In some embodiments, the targeting peptide has a sequence selected from: X₁X₂X₃RGDHVNL (SEQ ID NO: 98924); X₁X₂X₃RGDLIGR (SEQ ID NO: 98925); X₁X₂X₃RGDQSTL (SEQ ID NO: 98926); X₁X₂X₃RGDRGQI (SEQ ID NO: 98927); X₁X₂X₃RGDRGVV (SEQ ID NO: 98928); X₁X₂X₃RGDRQGI (SEQ ID NO: 98929); X₁X₂X₃RGDRSQT (SEQ ID NO: 98930); X₁X₂X₃RGDRSVV (SEQ ID NO: 98931); X₁X₂X₃RGDLLLS (SEQ ID NO: 98932); X₁X₂X₃RGDFNNL (SEQ ID NO: 98933); X₁X₂X₃RGDFQNT (SEQ ID NO: 98934); X₁X₂X₃RGDLTVT (SEQ ID NO: 98935); X₁X₂X₃RGDYVGL (SEQ ID NO: 98936); X₁X₂X₃RGDYSSV (SEQ ID NO: 98937); X₁X₂X₃RGDHGVL (SEQ ID NO: 98938); X₁X₂X₃RGDRDYL (SEQ ID NO: 98939); X₁X₂X₃RGDYNSL (SEQ ID NO: 98940); X₁X₂X₃RGDYTSV (SEQ ID NO: 98941); X₁X₂X₃RGDYTSM (SEQ ID NO: 98942); and X₁X₂X₃RGDFNNT (SEQ ID NO: 98943). In some embodiments, X₁ is selected from S, E, A, D, N, Q, or T. In some embodiments, X₁ is selected from S or E. In some embodiments, X₁ is S. In some embodiments, X₁ is E. In some embodiments, X₂ is selected from N, A, D, E, F, G, H, I, K, L, M, N, P, Q, R, S, T, V, W, or Y. In some embodiments, X₂ is selected from N or A. In some embodiments, X₂ is N. In some embodiments, X₂ is A. In some embodiments, X₃ is selected from R, Q, A, D, E, F, G, H, I, K, L, M, N, P, S, T, V, W, or Y. In some embodiments, X₃ is selected from R or Q. In some embodiments. X₃ is R. In some embodiments, X₃ is Q. In some embodiments, X₂ is N; and X₃ is R. In some embodiments, X₁ is E; X₂ is N; and X₃ is R. In some embodiments, X₁ is S; X₂ is N; and X₃ is R.

In some embodiments, the targeting peptide is positioned between 565 and 595 within VR VIII of the modified AAV capsid protein.

In some embodiments,
the reference AAV capsid protein is a capsid protein of AAV1 or a modification thereof and the targeting peptide is between Q585 and T589 of the reference AAV capsid protein;
the reference AAV capsid protein is a capsid protein of AAV2 or a modification thereof and the targeting peptide is between Q584 and R588 or between N587 and R588 of the reference AAV capsid protein;
the reference AAV capsid protein is a capsid protein of AAV3 or a modification thereof and the targeting peptide is between Q585 and T589 of the reference AAV capsid protein;
the reference AAV capsid protein is a capsid protein of AAV4 or a modification thereof and the targeting peptide is between G581 and N585 of the reference AAV capsid protein;
the reference AAV capsid protein is a capsid protein of AAV5 or a modification thereof and the targeting peptide is between Q574 and T578 of the reference AAV capsid protein;
the reference AAV capsid protein is a capsid protein of AAV6 or a modification thereof and the targeting peptide is between Q585 and T589 of the reference AAV capsid protein;
the reference AAV capsid protein is a capsid protein of AAV7 or a modification thereof and the targeting peptide is between Q586 and T590 of the reference AAV capsid protein;
the reference AAV capsid protein is a capsid protein of AAV8 or a modification thereof and the targeting peptide is between Q587 and A591 of the modified AAV capsid protein;
the reference AAV capsid protein is a capsid protein of AAV9 or a modification thereof and the targeting peptide is between H587 and A591 of the reference AAV capsid protein;
the reference AAV capsid protein is a capsid protein of AAVrh10 or a modification thereof and the targeting peptide is between Q587 and A591 of the reference AAV capsid protein;
the reference AAV capsid protein is a capsid protein of AAVpo.1 or a modification thereof and the targeting peptide is between N564 and S568 of the reference AAV capsid protein;
the reference AAV capsid protein is a capsid protein of AAV12 or a modification thereof and the targeting peptide is between N589 and A593 of the reference AAV capsid protein;
the reference AAV capsid protein is a capsid protein of Anc80 or a modification thereof and the targeting peptide is between Q585 and T589 of the reference AAV capsid protein;
the reference AAV capsid protein is a capsid protein of Anc80L65 or a modification thereof and the targeting peptide is between Q585 and T589 of the reference AAV capsid protein;
the reference AAV capsid protein is a capsid protein of Anc80-55 or a modification thereof and the targeting peptide is between Q585 and T589 of the reference AAV capsid protein;
the reference AAV capsid protein is a capsid protein of Anc80-129 or a modification thereof and the targeting peptide is between Q585 and T589 of the reference AAV capsid protein;
the reference AAV capsid protein is a capsid protein of Anc80-156 or a modification thereof and the targeting peptide is between Q585 and T589 of the reference AAV capsid protein;
the reference AAV capsid protein is a capsid protein of Anc80-751 or a modification thereof and the targeting peptide is between Q585 and T589 of the reference AAV capsid protein;
the reference AAV capsid protein is a capsid protein of Ane80-1029 or a modification thereof and the targeting peptide is between Q585 and T589 of the reference AAV capsid protein; or
the reference AAV capsid protein is a capsid protein of Anc80-1712 or a modification thereof and the targeting peptide is between Q585 and T589 of the reference AAV capsid protein.

In some embodiments,
the reference AAV capsid protein is a capsid protein of AAV1 or a modification thereof and the targeting peptide is between D590 and P591 or between S588 and T589 of the reference AAV capsid protein;
the reference AAV capsid protein is a capsid protein of AAV2 or a modification thereof and the targeting peptide is between R588 and Q589 or between N587 and R588 of the reference AAV capsid protein;
the reference AAV capsid protein is a capsid protein of AAV3 or a modification thereof and the targeting peptide is between S586 and S587 or between N588 and T589 of the reference AAV capsid protein;
the reference AAV capsid protein is a capsid protein of AAV4 or a modification thereof and the targeting peptide is between S584 and N585 or between S586 and N587 of the reference AAV capsid protein;
the reference AAV capsid protein is a capsid protein of AAV5 or a modification thereof and the targeting peptide is between S575 and S576 or between T577 and T578 of the reference AAV capsid protein;
the reference AAV capsid protein is a capsid protein of AAV6 or a modification thereof and the targeting peptide is between D590 and P591 or S588 and T589 of the reference AAV capsid protein;
the reference AAV capsid protein is a capsid protein of AAV7 or a modification thereof and the targeting peptide is between N589 and T590 of the reference AAV capsid protein;
the reference AAV capsid protein is a capsid protein of AAV8 or a modification thereof and the targeting peptide is between N590 and T591 of the modified AAV capsid protein;
the reference AAV capsid protein is a capsid protein of AAV9 or a modification thereof and the targeting peptide is between Q588 and A589 of the reference AAV capsid protein;
the reference AAV capsid protein is a capsid protein of AAVrh10 or a modification thereof and the targeting peptide is between N590 and A591 of the reference AAV capsid protein;
the reference AAV capsid protein is a capsid protein of AAVpo.1 or a modification thereof and the targeting peptide is between N567 and S568 or between N569 and T570 of the reference AAV capsid protein;
the reference AAV capsid protein is a capsid protein of AAV12 or a modification thereof and the targeting peptide is between N592 and A593 or between TS94 and T595 of the reference AAV capsid protein;
the reference AAV capsid protein is a capsid protein of Anc80 or a modification thereof and the targeting peptide is between T589 and A590 or between N587 and T588 of the reference AAV capsid protein;
the reference AAV capsid protein is a capsid protein of Anc80165 or a modification thereof and the targeting peptide is between T589 and A590 or between N587 and T588 of the reference AAV capsid protein;
the reference AAV capsid protein is a capsid protein of Anc80-55 or a modification thereof and the targeting peptide is between T589 and A590 or between N587 and T588 of the reference AAV capsid protein;
the reference AAV capsid protein is a capsid protein of Anc80-129 or a modification thereof and the targeting peptide is between T589 and A590 or between NS87 and T588 of the reference AAV capsid protein;
the reference AAV capsid protein is a capsid protein of Ane80-156 or a modification thereof and the targeting peptide is between T589 and A590 or between N587 and T588 of the reference AAV capsid protein;
the reference AAV capsid protein is a capsid protein of Anc80-751 or a modification thereof and the targeting peptide is between T589 and A590 or between N587 and T588 of the reference AAV capsid protein;
the reference AAV capsid protein is a capsid protein of Anc80-1029 or a modification thereof and the targeting peptide is between T589 and A590 or between N587 and T588 of the reference AAV capsid protein; or
the reference AAV capsid protein is a capsid protein of Anc80-1712 or a modification thereof and the targeting peptide is between T589 and A590 or between N587 and T588 of the reference AAV capsid protein.

In some embodiments,
P₁ is independently selected from an asparagine (N), a serine (S), or a threonine (T);
P₂ is independently selected from a serine (S) or a glycine (G);
P₃ is independently selected from a threonine (T), a glutamine (Q), an alanine (A), or glutamate (E);
P₄ is independently selected from a serine (S), a threonine (T), or an alanine (A);
P₅ is independently selected from a glycine (G) or an alanine (A);
P₆ is independently selected from a glycine (G) or an alanine (A);
P₇ is independently selected from an alanine (A) or a serine (S);
P₈ is independently selected from a serine (S) or a threonine (T); and
P₁₂ is independently selected from a histidine (H), a threonine (T), or an alanine (A).

In some embodiments, P₅, P₆ or both P₅ and P₆ are not an alanine (A).

In some embodiments, the peptide segment does not comprise an alanine (A) at P₆ and a threonine (T) at P₈.

In some embodiments, the peptide segment has a sequence of P₁P₂P₃P₄GGP₇P₈NDNP₁₂ (SEQ ID NO: 44921), wherein P₁, P₂, P₃, P₄, P₇, P₈, and P₁₂ are independently selected from any amino acid residue.

In some embodiments,
P₁ is independently selected from an asparagine (N) or a serine (S);
P₂ is independently selected from a serine (S) or a glycine (G);
P₃ is independently selected from a threonine (T) or a glutamine (Q);
P₄ is independently selected from a serine (S), a threonine (T), or an alanine (A);
P₇ is independently selected from an alanine (A) or a serine (S);
P₈ is independently selected from a serine (S) or a threonine (T); and
P₁₂ is independently selected from a histidine (H), a threonine (T), or an alanine (A).

In some embodiments, the peptide segment has a sequence of P₁P₂TP₄GGP₇P₈NDNP₁₂ (SEQ ID NO: 44922), wherein P₁, P₂, P₄, P₇, P₈, and P₁₂ are independently selected from any amino acid residue.

In some embodiments, the peptide segment has a sequence of P₁P₂QP₄GGP₇P₈NDNP₁₂ (SEQ ID NO: 44923), wherein P₁, P₂, P₄, P₇, P₈, and P₁₂ are independently selected from any amino acid residue.

In some embodiments, the peptide segment has a sequence of P₁P₂TP₄GGP₇TNDNP₁₂ (SEQ ID NO: 44924), wherein P₁, P₂, P₄, P₇, and P₁₂ are independently selected from any amino acid residue.

In some embodiments, the peptide segment has a sequence of NP₂TP₄GGP₇P₈NDNP₁₂ (SEQ ID NO: 44925), wherein P₂, P₄, P₇, and P₁₂ are independently selected from any amino acid residue.

In some embodiments, the peptide segment has a sequence of SP₂TP₄GGP₇P₈NDNP₁₂ (SEQ ID NO: 44926), wherein P₂, P₄, P₇, P₈, and P₁₂ are independently selected from any amino acid residue.

In some embodiments, the peptide segment is NSTSGGP₇P₈NDNH (SEQ ID NO: 44927), wherein P₇ and P₈ are independently selected from any amino acid residue.

In some embodiments, P₇ is independently selected from an alanine (A) or a serine (S) and P₈ is independently selected from a serine (S) or a threonine (T).

In some embodiments, the peptide segment comprises:

(SEQ ID NO: 46026)
NSTSGGASNDNH,
(SEQ ID NO: 46029)
NSTSGGATNDNH,
(SEQ ID NO: 46031)
NSTSGGSSNDNH,
or
(SEQ ID NO: 46034)
NSTSGGSTNDNH.

In some embodiments, the peptide segment is NSTTGGP₇P₈NDNH (SEQ ID NO: 44928), wherein P₇ and P₈ are independently selected from any amino acid residue.

In some embodiments, P₇ is independently selected from an alanine (A) or a serine (S) and P₈ is independently selected from a serine (S) or a threonine (T).

In some embodiments, the peptide segment comprises:

(SEQ ID NO: 46073)
NSTTGGASNDNH,
(SEQ ID NO: 46076)
NSTTGGATNDNH,
(SEQ ID NO: 46079)
NSTTGGSSNDNH,
or
(SEQ ID NO: 46082)
NSTTGGSTNDNH.

In some embodiments, the peptide segment is SGQTGGP₇P₈NDNH (SEQ ID NO: 44929), wherein P₇ and P₈ are independently selected from any amino acid residue.

In some embodiments, P₇ is independently selected from an alanine (A) or a serine (S) and P₈ is independently selected from a serine (S) or a threonine (T).

In some embodiments, the peptide segment comprises:

(SEQ ID NO: 46505)
SGQTGGASNDNH,
(SEQ ID NO: 46508)
SGQTGGATNDNH,
(SEQ ID NO: 46511)
SGQTGGSSNDNH,
or
(SEQ ID NO: 46514)
SGQTGGSTNDNH.

In some embodiments, the peptide segment is SGTAGGP₇P₈NDNT (SEQ ID NO: 44930), wherein P₇ and P₈ are independently selected from any amino acid residue.

In some embodiments, P₇ is independently selected from an alanine (A) or a serine (S) and P₈ is independently selected from a serine (S) or a threonine (T).

In some embodiments, the peptide segment comprises:

(SEQ ID NO: 46554)
SGTAGGASNDNT,
or
(SEQ ID NO: 46560)
SGTAGGSSNDNT.

In some embodiments, the peptide segment does not comprise SGTAGGATNDNT (SEQ ID NO: 46557) or SGTAGGSTNDNT (SEQ ID NO: 46563).

In some embodiments, the peptide segment is SGTSGGP₇P₈NDNA (SEQ ID NO: 44931), wherein P₇ and P₈ are independently selected from any amino acid residue.

In some embodiments, P₇ is independently selected from an alanine (A) or a serine (S) and P₈ is independently selected from a serine (S) or a threonine (T).

In some embodiments, the peptide segment comprises:

(SEQ ID NO: 46600)
SGTSGGASNDNA,
(SEQ ID NO: 46603)
SGTSGGATNDNA,
(SEQ ID NO: 46606)
SGTSGGSSNDNA,
or
(SEQ ID NO: 46609)
SGTSGGSTNDNA.

In some embodiments, the peptide segment is SGTTGGP₇P₈NDNT (SEQ ID NO: 44932), wherein P₇ and P₈ are independently selected from any amino acid residue.

In some embodiments, P₇ is independently selected from an alanine (A) or a serine (S) and P₈ is independently selected from a serine (S) or a threonine (T).

In some embodiments, the peptide segment comprises:

(SEQ ID NO: 46650)
SGTTGGASNDNT,
(SEQ ID NO: 46653)
SGTTGGATNDNT,
(SEQ ID NO: 46656)
SGTTGGSSNDNT,
or
(SEQ ID NO: 46659)
SGTTGGSTNDNT.

In some embodiments, the peptide segment is SSTAGGP₇P₈NDNA (SEQ ID NO: 44933), wherein P₇ and P₈ are independently selected from any amino acid residue.

The In some embodiments, P₇ is independently selected from an alanine (A) or a serine (S) and P₈ is independently selected from a serine (S) or a threonine (T).

In some embodiments, the peptide segment comprises:

(SEQ ID NO: 47128)
SSTAGGASNDNA,
(SEQ ID NO: 47131)
SSTAGGATNDNA,
(SEQ ID NO: 47134)
SSTAGGSSNDNA,
or
(SEQ ID NO: 47137)
SSTAGGSTNDNA.

In some embodiments, the peptide segment is SSTAGGASNDNA (SEQ ID NO: 47128).

In some embodiments, the peptide segment is SSTAGGATNDNA (SEQ ID NO: 47131).

In some embodiments, the peptide segment is NSTSGASTNDNA (SEQ ID NO: 48390).

In some embodiments, the peptide segment is selected from a peptide segment as shown in Tables 20 or 28.

In some embodiments, variable region I (VR I) corresponds to amino acid residues between about position 259 to about position 275 of the modified capsid protein.

In some embodiments, the peptide segment is at a position between S261 and Y274 of an AAV9 capsid protein (SEQ ID NO: 61).

In some embodiments, the peptide segment is at a position between S260 and Y273 of an Anc80 capsid protein (SEQ ID NO: 132).

In some embodiments, the peptide segment is at a position between S260 and Y273 of an Ane801.65 capsid protein (SEQ ID NO: 142).

In some embodiments, the peptide segment is at a position between S260 and Y273 of an AAV2 capsid protein (SEQ ID NO: 55).

In some embodiments, the targeting peptide has an amino acid sequence selected from SEQ ID NOs: 44864-44867, 44879-44883, 44911, 44912, 44913, and 44918-44919; and the peptide segment has an amino acid sequence selected from SEQ ID NOs: 46026, 46029, 46031, 46035, 46073, 46076, 46079, 46082, 46505, 46508, 46511, 46514, 46554, 46560, 46609, 46600, 46603, 46606, 46650, 46653, 46656, 46659, 47128, 47131, 47134, and 47137.

In some embodiments, the targeting peptide has an amino acid sequence selected from SEQ ID NOs: 44864-44867, 44879-44883, 44911, 44912, 44913, and 44918-44919; and the peptide segment has the amino acid sequence SGTAGGASNDNT (SEQ ID NO: 46554).

In some embodiments, the targeting peptide has an amino acid sequence selected from SEQ ID NOs: 44864-44867, 44879-44883, 44911, 44912, 44913, and 44918-44919; and the peptide segment has the amino acid sequence SGTSGGSTNDNA (SEQ ID NO: 46609).

In some embodiments, the targeting peptide has an amino acid sequence selected from SEQ ID NOs: 44864-44867, 44879-44883, 44911, 44912, 44913, and 44918-44919; and the peptide segment has the amino acid sequence SGTTGGSTNDNT (SEQ ID NO: 46659).

In some embodiments, the targeting peptide has an amino acid sequence selected from SEQ ID NOs: 44864-44867, 44879-44883, 44911, 44912, 44913, and 44918-44919; and the peptide segment has the amino acid sequence SGTTGGSTNDNT (SEQ ID NO: 47128).

In some embodiments, the targeting peptide has an amino acid sequence selected from SEQ ID NOs: 44864-44867, 44879-44883, 44911, 44912, 44913, and 44918-44919; and the peptide segment has the amino acid sequence NSTSGGSSNDNA (SEQ ID NO: 48388).

In some embodiments, the targeting peptide has an amino acid sequence selected from SEQ ID NOs: 44864-44867, 44879-44883, 44911, 44912, 44913, and 44918-44919; and the peptide segment has the amino acid sequence NSTSGASTNDNA (SEQ ID NO: 48390).

In some embodiments, the targeting peptide has the amino acid sequence ENRRGDENNL (SEQ ID NOs: 44864); and the peptide segment has the amino acid sequence SGTTGGSTNDNT (SEQ ID NO: 46659).

In some embodiments, the targeting peptide has the amino acid sequence ENRRGDFNNL (SEQ ID NOs: 44864); and the peptide segment has the amino acid sequence SGTTGGSSNDNT (SEQ ID NO: 46656).

In some embodiments, the targeting peptide has the amino acid sequence ENRRGDFNNL (SEQ ID NOs: 44864); and the peptide segment has the amino acid sequence SSTAGGASNDNA (SEQ ID NO: 47128).

In some embodiments, the targeting peptide has the amino acid sequence SAQRGDLLLS (SEQ ID NO: 44882); and the peptide segment has the amino acid sequence SSTAGGATNDNA (SEQ ID NO: 47131).

In some embodiments, the targeting peptide has the amino acid sequence SAQRGDLLLS (SEQ ID NO: 44882); and the peptide segment has the amino acid sequence SGTTGGSTNDNT (SEQ ID NO: 46659).

In some embodiments, the targeting peptide has the amino acid sequence SAQRGDLLLS (SEQ ID NO: 44882); and the peptide segment has the amino acid sequence SGTTGGSSNDNT (SEQ ID NO: 46656).

In some embodiments, the targeting peptide has the amino acid sequence SAQRGDLLLS (SEQ ID NO: 44882); and the peptide segment has the amino acid sequence SSTAGGASNDNA (SEQ ID NO: 47128).

In some embodiments, the targeting peptide has the amino acid sequence ENRRGDFQNT (SEQ ID NO: 44866); and the peptide segment has the amino acid sequence SSTAGGATNDNA (SEQ ID NO: 47131).

In some embodiments, the targeting peptide has the amino acid sequence ENRRGDFQNT (SEQ ID NO: 44866); and the peptide segment has the amino acid sequence SGTTGGSTNDNT (SEQ ID NO: 46659).

In some embodiments, the targeting peptide has the amino acid sequence ENRRGDFQNT (SEQ ID NO: 44866); and the peptide segment has the amino acid sequence SGTTGGSSNDNT (SEQ ID NO: 46656).

In some embodiments, the targeting peptide has the amino acid sequence SNRRGDFNNT (SEQ ID NO: 44883); and the peptide segment has the amino acid sequence SSTAGGASNDNA (SEQ ID NO: 47128).

In another aspect, this disclosure features a modified adeno-associated virus (AAV) capsid protein, comprising: a targeting peptide at a site within VR VIII, wherein the targeting peptide has a sequence selected from SEQ ID NOs: 44864-44867, 44879-44883, 44911, 44912, 44913, and 44918-44919; and a peptide segment within VR I, wherein the peptide segment has a sequence selected from SEQ ID NOs: 46026, 46029, 46031, 46035, 46073, 46076, 46079, 46082, 46505, 46508, 46511, 46514, 46554, 46560, 46609, 46600, 46603, 46606, 46650, 46653, 46656, 46659, 47128, 47131, 47134, and 47137.

In another aspect, this disclosure features a polynucleotide encoding any of the modified AAV capsid proteins described herein.

In another aspect, this disclosure features a vector comprising any of the polynucleotides described herein.

In some embodiments, the vector also includes a promoter operably linked to the polynucleotide.

In another aspect, this disclosure features a host cell comprising any of the modified AAV capsid proteins described herein, any of the polynucleotides described herein, or any of the vectors described herein.

In another aspect, this disclosure features a recombinant AAV virion (rAAV) any of the modified AAV capsid proteins described herein.

In some embodiments, the AAV virion also includes an exogenous polynucleotide.

In some embodiments, the exogenous polynucleotide comprises a template for homology directed repair.

In some embodiments, the exogenous polynucleotide comprises an expressible polynucleotide encoding a therapeutic tRNA, miRNA, gene editing guide RNA, or RNA-editing guide RNA.

In some embodiments, the exogenous polynucleotide comprises an expressible polynucleotide encoding a therapeutic protein.

In some embodiments, the therapeutic protein is MTM1 or a fragment thereof.

In some embodiments, the expressible polypeptide comprises the sequence of SEQ ID NO: 165 or a fragment thereof.

In some embodiments, the expressible polypeptide comprises the sequence having at least 80%, 85%, 90%, 95%, 98%, 99% or 100% sequence identity to any of SEQ ID Nos: 166-170.

In some embodiments, the exogenous polynucleotide further comprises a regulatory sequence. In some embodiments, the regulatory sequence comprises expression regulatory elements (EREs). In some embodiments, the EREs comprise a CAG promoter. In some embodiments, the EREs comprise a sequence having at least 90%, at least 95%, at least 98%, at least 99% or 100% sequence identity to any one of SEQ IDs NO: 171-173.

In another aspect, this disclosure features a pharmaceutical composition comprising any of the modified AAV capsid proteins described herein or any of the rAAV virions described herein.

In another aspect, this disclosure features a method for treating or ameliorating or preventing a disease or condition in a subject, comprising administering a therapeutically effective amount of any of the AAV virions described herein or any of the pharmaceutical compositions described herein.

In some embodiments of any of the methods of treating or ameliorating or preventing a disease as described herein, the disease is a muscular disease and/or the condition is muscle degeneration.

In some embodiments of any of the methods of treating or ameliorating or preventing a disease as described herein, said muscle is a striated muscle, preferably heart or a skeletal muscle or diaphragm.

In some embodiments of any of the methods of treating or ameliorating or preventing a disease as described herein, wherein said muscular disease is a muscular dystrophy, a cardiomyopathy, a myotonia, a muscular atrophy, a myoclonus dystonia, a mitochondrial myopathy, a rhabdomyolysis, a fibromyalgia, and/or a myofascial pain syndrome.

In some embodiments, any of the modified adeno-associated virus (AAV) capsid proteins described herein for use in treating and/or preventing a muscular disease and/or muscle degeneration.

In another aspect, this disclosure features an AAV virion comprising any of the modified AAV capsid proteins described herein or any of the rAAV virions described herein for use in treating and/or preventing a muscular disease and/or in muscle regeneration.

In another aspect, this disclosure features a pharmaceutical composition comprising any of the modified AAV capsid proteins described herein, and/or any of the rAAV virions described herein for use in treating and/or preventing a muscular disease and/or in muscle regeneration.

In another aspect, this disclosure features a method of transferring an exogenous polynucleotide into a muscle cell, comprising the step of administering any of the rAAV virions described herein to a subject.

In some embodiments, the administration results in transfer of the exogenous polynucleotide in the muscle cell, at a muscle:liver infection ratio of greater than 1 when measured by genome copies of the AAV virion.

In some embodiments, the muscle:liver infection ratio ranges from 1 to 100. In some embodiments, the muscle:liver infection ration ranges from 1 to 10.

In some embodiments, the muscle:liver infection ratio ranges from 2 to 8.

In some embodiments, the administration results in expression of the exogenous polynucleotide in the muscle cell, at a muscle:liver expression ratio of greater than 10.

In some embodiments, the muscle:liver expression ratio ranges from 10 to 100.

In some embodiments, the muscle:liver expression ratio ranges from 20 to 80.

In some embodiments, the muscle:liver expression ratio ranges from 50 to 80 when measured by mRNA transcript expression.

In some embodiments, the muscle:liver expression ratio ranges from 10 to 50 when measured by protein expression.

In some embodiments, the muscle cell is selected from triceps surae, biceps, heart and quadricep.

In another aspect, this disclosure features use of the any of the modified AAV capsid proteins described herein, and/or any of the AAV virions described herein for transferring an exogenous polynucleotide into a muscle cell.

In some embodiments, said use is a non-therapeutic use, preferably wherein said use is an in vitro use. In some embodiments, the muscle cell is selected from triceps surae, biceps, heart and quadricep.

Accordingly, one aspect of the present disclosure provides a modified adeno-associated virus (AAV) capsid protein, comprising: (i) a reference AAV capsid protein, and (ii) a 7-mer peptide having the sequence RGDLLLS (SEQ ID NO: 1) inserted into a site within VR VIII of the reference AAV capsid protein.

In some embodiments, the AAV capsid protein is selected from one or more of VP1, VP2 and VP3. In some embodiments, the reference AAV capsid protein is a capsid protein of an AAV variant selected from the group consisting of: AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B, rh.64-B; hu.27-B; hu.21-B; hu.22-B; hu.23-B; hu.7-C; hu.61-C; rh.56-C; hu.9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80-55, Anc80-129, Anc80-156, Anc80-751, Anc80-1029, Anc80-1712, Anc80L44; Anc80L1; Anc110; and Anc80DI. In some embodiments, the reference AAV capsid protein is a capsid protein having a sequence selected from SEQ ID Nos: 54-152 or a fragment thereof.

In some embodiments, the 7-mer peptide is inserted into an amino acid position between 565 and 595 of the reference AAV capsid protein. In some embodiments, (i) the reference AAV capsid protein is a capsid protein of AAV1 and the 7-mer peptide is inserted between D590 and P591 or between S588 and T589 of the capsid protein; (ii) the reference AAV capsid protein is a capsid protein of AAV2 and the 7-mer peptide is inserted between R588 and Q589 or between N587 and R588 of the capsid protein; (iii) the reference AAV capsid protein is a capsid protein of AAV3b and the 7-mer peptide is inserted between S586 and S587 or between N588 and T589 of the capsid protein; (iv) the reference AAV capsid protein is a capsid protein of AAV4 and the 7-mer peptide is inserted between S584 and N585 or between S586 and N587 of the capsid protein; (v) the reference AAV capsid protein is a capsid protein of AAV5 and the 7-mer peptide is inserted between S575 and S576 or between T577 and T578 of the capsid protein; (vi) the reference AAV capsid protein is a capsid protein of AAV6 and the 7-mer peptide is inserted between D590 and P591 or S588 and TS89 of the capsid protein; (vii) the reference AAV capsid protein is a capsid protein of AAV7 and the 7-mer peptide is inserted between N589 and T590 of the capsid protein; (viii) the reference AAV capsid protein is a capsid protein of AAV8 and the 7-mer peptide is inserted between N590 and T591 of the capsid protein; (ix) the reference AAV capsid protein is a capsid protein of AAV9 and the 7-mer peptide is inserted between Q588 and A589 of the capsid protein; (x) the reference AAV capsid protein is a capsid protein of AAVrh10 and the 7-mer peptide is inserted between N590 and A591 of the capsid protein; (vi) the reference AAV capsid protein is a capsid protein of AAVpo.1 and the 7-mer peptide is inserted between N567 and S568 or between N569 and TS70 of the capsid protein; or (xii) the reference AAV capsid protein is a capsid protein of AAV12 and the 7-mer peptide is inserted between N592 and A593 or between T594 and T595 of the capsid protein.

In some embodiments, the modified AAV capsid protein has a sequence of SEQ ID NO: 158.

In some embodiments, the reference AAV capsid protein is a liver-toggle mutant of a capsid protein of an AAV variant selected from the group consisting of: AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu.9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Ane82; Anc83; Anc84; Anc94; Ane113; Ane126; Anc127; Anc80L27; Anc80L59; Anc80L60; Anc80L62; Anc80L65; Anc80L33; Anc80L36; Anc80L44; Anc80L1; Anc80-55, Anc80-129, Anc80-156, Anc80-751, Anc80-1029, Anc80-1712, Anc110; and Anc80DI. In some embodiments, the reference AAV capsid protein is a liver-toggle mutant of a capsid protein having a sequence selected from SEQ ID Nos: 54-152 or a fragment thereof.

In some embodiments, the modified AAV capsid protein comprises an alanine (A) amino acid residue at an amino acid position corresponding to position 266 in Anc80; or a lysine (K) amino acid residue at an amino acid position corresponding to position 168 in Anc80.

In some embodiments, the modified AAV capsid protein comprises a glycine (G) amino acid residue at an amino acid position corresponding to position 266 in Anc80; or an arginine (R) amino acid residue at an amino acid position corresponding to position 168 in Anc80.

In some embodiments, the reference AAV capsid protein is a liver toggle mutant of a capsid protein of AAV9 comprising an alanine (A) amino acid residue at an amino acid position 267 and a threonine (T) amino acid residue at an amino acid position 269. In some embodiments, the modified AAV capsid protein comprises the sequence of SEQ ID NO: 159.

In some embodiments, the modified AAV capsid protein comprises a glycine (G) amino acid residue at an amino acid position corresponding to position 266 in Anc80; or a lysine (K) amino acid residue at an amino acid position corresponding to position 168 in Anc80.

In another aspect, the present disclosure provides a modified adeno-associated virus (AAV) capsid protein, comprising: (i) a liver-toggle mutant of a reference AAV capsid protein, comprising a) an alanine (A) or glycine (G) amino acid residue at an amino acid position corresponding to position 266 in Ane80; or b) a lysine (K) or arginine (R) amino acid residue at an amino acid position corresponding to position 168 in Anc80; and (ii) a targeting peptide inserted into a site within VR VIII of the liver-toggle mutant.

In some embodiments, the liver-toggle mutant comprises: a) an alanine (A) amino acid residue at an amino acid position corresponding to position 266 in Anc80; or b) a lysine (K) amino acid residue at an amino acid position corresponding to position 168 in Anc80. In some embodiments, the liver-toggle mutant comprises: a) an alanine (A) amino acid residue at an amino acid position corresponding to position 266 in Anc80; and b) a lysine (K) amino acid residue at an amino acid position corresponding to position 168 in Anc80.

In some embodiments, the liver-toggle mutant comprises: a) a glycine (G) amino acid residue at an amino acid position corresponding to position 266 in Anc80; or b) an arginine (R) amino acid residue at an amino acid position corresponding to position 168 in Anc80. In some embodiments, the liver-toggle mutant comprises: a) a glycine (G) amino acid residue at an amino acid position corresponding to position 266 in Anc80; and b) an arginine (R) amino acid residue at an amino acid position corresponding to position 168 in Anc80.

In some embodiments, the targeting peptide is 7-mer peptide having the sequence RGDX₁X₂X₃X₄, wherein X₁ to X₄ are independently selected amino acid residues. In some embodiments, X₁, X₂, and X₃ are independently selected from L, G, V, and A; and X₄ is selected from S, V, A, G, and L. In some embodiments, X₁, X₂, and X₃ are independently selected from L, V, and A; and at least two of X₁, X₂, and X₃ are independently L. In some embodiments, X₂ is L. In some embodiments, 7-mer peptide has a sequence of RGDLLLS (SEQ ID NO: 1).

In some embodiments, the targeting peptide is the 7-mer peptide TLAVPFK (SEQ ID NO: 53). In some embodiments, the targeting peptide has a sequence selected from SEQ ID Nos: 2-51 and 53.

In some embodiments, the reference AAV capsid protein is a capsid protein of an AAV variant selected from the group consisting of: AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu.9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc80-55; Anc80-129. Anc80-156; Anc80-751; Anc80-1029; Anc80-1712. Anc110; and Anc80DI. In some embodiments, the reference AAV capsid protein is a capsid protein having a sequence selected from SEQ ID Nos: 54-152 or a fragment thereof.

In some embodiments, the reference AAV capsid polypeptide is an AAV9 capsid protein.

In some embodiments, the liver-toggle mutant comprises an alanine (A) amino acid residue at position 267. In some embodiments, the liver-toggle mutant comprises a threonine (T) amino acid residue at position 269. In some embodiments, the liver-toggle mutant comprises an alanine (A) amino acid residue at position 267 and a threonine (T) amino acid residue at position 269.

In some embodiments, the targeting peptide is inserted into an amino acid position between 565 and 595 of the liver toggle mutant. In some embodiments, (i) the reference AAV capsid protein is a capsid protein of AAV1 and the targeting peptide is inserted between D590 and P591 or between S588 and T589 of the liver-toggle mutant; (ii) the reference AAV capsid protein is a capsid protein of AAV2 and the targeting peptide is inserted between R588 and Q589 or between N587 and R588 of the liver-toggle mutant; (iii) the reference AAV capsid protein is a capsid protein of AAV3b and the targeting peptide is inserted between S586 and S587 or between N588 and T589 of the liver-toggle mutant; (iv) the reference AAV capsid protein is a capsid protein of AAV4 and the targeting peptide is inserted between S584 and N585 or between S586 and N587 of the liver-toggle mutant; (v) the reference AAV capsid protein is a capsid protein of AAV5 and the targeting peptide is inserted between S575 and S576 or between T577 and T578 of the liver-toggle mutant; (vi) the reference AAV capsid protein is a capsid protein of AAV6 and the targeting peptide is inserted between D590 and P591 or S588 and T589 of the liver-toggle mutant; (vii) the reference AAV capsid protein is a capsid protein of AAV7 and the targeting peptide is inserted between N589 and TS90 of the liver-toggle mutant; (viii) the reference AAV capsid protein is a capsid protein of AAV8 and the targeting peptide is inserted between N590 and T591 of the liver-toggle mutant; (ix) the reference AAV capsid protein is a capsid protein of AAV9 and the targeting peptide is inserted between Q588 and A589 of the liver-toggle mutant; (x) the reference AAV capsid protein is a capsid protein of AAVrh10 and the targeting peptide is inserted between N590 and A591 of the liver-toggle mutant; (xi) the reference AAV capsid protein is a capsid protein of AAVpo.1 and the targeting peptide is inserted between N567 and S568 or between NS69 and T570 of the liver-toggle mutant; or (xii) the reference AAV capsid protein is a capsid protein of AAV12 and the targeting peptide is inserted between N592 and A593 or between T594 and T595 of the liver-toggle mutant.

In some embodiments, the liver-toggle mutant comprises a sequence selected from NSTSGASS (SEQ ID NO: 160), NSTSGGST (SEQ ID NO: 161) and NSTSGAST (SEQ ID NO: 162).

In some embodiments, the liver-toggle mutant of a reference AAV capsid protein, comprises a) an alanine (A) amino acid residue at an amino acid position corresponding to position 266 in Anc80; and b) a lysine (K) amino acid residue at an amino acid position corresponding to position 168 in Anc80

In some embodiments, the liver-toggle mutant of a reference AAV capsid protein, comprises a) an alanine (A) amino acid residue at an amino acid position corresponding to position 267 in AAV9; and b) a threonine (T) amino acid residue at an amino acid position corresponding to position 269 in AAV9.

In some embodiments, the liver-toggle mutant further comprises a) an alanine (A) amino acid residue at an amino acid position corresponding to position 504 in AAV9; and b) an alanine (A) amino acid residue at an amino acid position corresponding to position 505 in AAV9.

In some embodiments, the modified AAV capsid protein comprises a sequence of SEQ ID NO: 159.

In yet another aspect, the present disclosure provides a polynucleotide encoding the modified AAV capsid protein disclosed herein. In one aspect, the present disclosure relates to a vector comprising the polynucleotide. In some embodiments, the vector further comprises a promoter operably linked to the polynucleotide. Further disclosed herein includes a host cell comprising the modified AAV capsid protein, the polynucleotide, or the vector.

One aspect of the present disclosure provides a recombinant AAV virion (rAAV) comprising the modified AAV capsid protein disclosed herein. In some embodiments, the rAAV virion further comprises an exogenous polynucleotide. In some embodiments, the exogenous polynucleotide comprises a template for homology directed repair. In some embodiments, the exogenous polynucleotide comprises an expressible polynucleotide encoding a therapeutic tRNA, miRNA, gene editing guide RNA, or RNA-editing guide RNA. In some embodiments, the exogenous polynucleotide comprises an expressible polynucleotide encoding a therapeutic protein.

Another aspect of the present disclosure provides a pharmaceutical composition comprising the modified AAV capsid protein or the AAV virion.

It further discloses a method for treating or ameliorating or preventing a disease or condition in a subject, comprising administering a therapeutically effective amount of the AAV virion or the pharmaceutical composition of the present disclosure. In some embodiments, the disease is a muscular disease and/or the condition is muscle degeneration. In some embodiments, said muscle is a striated muscle, preferably heart or a skeletal muscle or diaphragm. In some embodiments, said muscular disease is a muscular dystrophy, a cardiomyopathy, a myotonia, a muscular atrophy, a myoclonus dystonia, a mitochondrial myopathy, a rhabdomyolysis, a fibromyalgia, and/or a myofascial pain syndrome.

In one aspect, the present disclosure provides a modified adeno-associated virus (AAV) capsid protein for use in treating and/or preventing a muscular disease and/or muscle degeneration. It further discloses an AAV virion comprising the modified AAV capsid protein for use in treating and/or preventing a muscular disease and/or in muscle regeneration. It also discloses a pharmaceutical composition comprising the modified AAV capsid protein, and/or the AAV virion for use in treating and/or preventing a muscular disease and/or in muscle regeneration. Additionally, provided herein includes use of the AAV capsid polypeptide, and/or the AAV virion for transferring an active compound into a muscle cell. In some embodiments, said use is a non-therapeutic use, preferably wherein said use is an in vitro use.

In one aspect, the present disclosure provides a method of transferring an exogenous polynucleotide into a muscle cell, comprising the step of administering the AAV virion of the present disclosure to a subject. In some embodiments, the administration results in transfer of the exogenous polynucleotide in the muscle cell, at a muscle:liver infection ratio of greater than 1 when measured by genome copies of the AAV virion. In some embodiments, the muscle:liver infection ratio ranges from 1 to 100. In some embodiments, the muscle:liver infection ration ranges from 1 to 10. In some embodiments, the muscle:liver infection ratio ranges from 2 to 8.

In some embodiments, the administration results in expression of the exogenous polynucleotide in the muscle cell, at a muscle:liver expression ratio of greater than 10. In some embodiments, the muscle:liver expression ratio ranges from 10 to 100. In some embodiments, the muscle:liver expression ratio ranges from 20 to 80. In some embodiments, the muscle:liver expression ratio ranges from 50 to 80 when measured by mRNA transcript expression. In some embodiments, the muscle:liver expression ratio ranges from 10 to 50 when measured by protein expression.

In some embodiments, the muscle cell is selected from triceps surae, biceps, heart and quadricep.

In another aspect, the present disclosure provides an rAAV whose genome comprises an MIMI coding sequence operably linked to an expression regulatory element (ERE); and one, two or all three of the following features: (a) the ERE is a hybrid expression regulatory element (ERE) comprising a CMV enhancer and a chicken beta actin promoter operably linked to the MTM1 coding sequence; and/or (b) the rAAV comprises a modified AAV capsid protein comprising at least one liver-toggle mutation and/or one muscle-targeting element; and/or (c) the MTM1 coding sequence is codon optimized for expression in human cells, optionally wherein the coding sequence has at least 90%, at least 95%, at least 98% or at least 99% sequence identity to any one of SEQ ID NOS: 167 to 170.

In some embodiments, the MTM1 sequence encodes a protein comprising an amino acid sequence having at least 95% sequence identity to the amino acid sequence of SEQ ID NO:164. In some embodiments, the MTM1 protein comprises an amino acid sequence having at least 98% or at least 99% sequence identity to the amino acid sequence of SEQ ID NO: 164. In some embodiments, the MTM1 protein comprises an amino acid sequence having 100% sequence identity to the amino acid sequence of SEQ ID NO: 164.

In some embodiments, the MTM1 sequence encodes a protein comprising an amino acid sequence having at least 95% sequence identity to the amino acid sequence of SEQ ID NO: 165. In some embodiments, the MTM1 protein comprises an amino acid sequence having at least 98% sequence identity to the amino acid sequence of SEQ ID NO: 165. In some embodiments, the MTM1 protein comprises an amino acid sequence having at least 99% sequence identity to the amino acid sequence of SEQ ID NO: 165. In some embodiments, the MTM1 protein comprises an amino acid sequence having 100% sequence identity to the amino acid sequence of SEQ ID NO: 165.

In some embodiments, the MTM1 coding sequence comprises a nucleotide sequence having at least 90% sequence identity to SEQ ID NO 166. In some embodiments, the MTM1 coding sequence comprises a nucleotide sequence having at least 95% sequence identity to SEQ ID NO: 166. In some embodiments, the MTM1 coding sequence comprises a nucleotide sequence having at least 98% sequence identity to SEQ ID NO: 166. In some embodiments, the MTM1 coding sequence comprises a nucleotide sequence having at least 99% sequence identity to SEQ ID NO: 166. In some embodiments, the MTM1 coding sequence comprises a nucleotide sequence having 100% sequence identity to SEQ ID NO: 166.

In some embodiments, the MTM1 coding sequence is codon optimized for expression in human cells. In some embodiments, the MTM1 coding sequence comprises a nucleotide sequence having at least 90% sequence identity to any one of SEQ ID NOS: 167 to 170. In some embodiments, the MTM1 coding sequence comprises a nucleotide sequence having at least 95% sequence identity to any one of SEQ ID NOS: 167 to 170. In some embodiments, the MTM1 coding sequence comprises a nucleotide sequence having at least 98% sequence identity to any one of SEQ ID NOS: 167 to 170. In some embodiments, the MTM1 coding sequence comprises a nucleotide sequence having at least 99% sequence identity to any one of SEQ ID NOS: 167 to 170. In some embodiments, the MTM1 coding sequence comprises a nucleotide sequence having 100% sequence identity to any one of SEQ ID NOS: 167 to 170. In some embodiments, the sequence identity is to SEQ ID NO: 167 In some embodiments, the sequence identity is to SEQ ID NO: 168. In some embodiments, the sequence identity is to SEQ ID NO: 169. In some embodiments, the sequence identity is to SEQ ID NO:169.

In some embodiments, the rAAV comprises a hybrid expression regulatory element (ERE) comprising a CMV enhancer and a chicken beta actin promoter operably linked to the MTM1 coding sequence.

In some embodiments, the ERE comprises (a) a nucleotide sequence having at least 90% sequence identity to SEQ ID NO:171 and a nucleotide sequence having at least 90% sequence identity to SEQ ID NO: 172 or (b) a nucleotide sequence having at least 90% sequence identity to SEQ ID NO: 173. In some embodiments, the ERE comprises (a) a nucleotide sequence having at least 95% sequence identity to SEQ ID NO: 171 and a nucleotide sequence having at least 95% sequence identity to SEQ ID NO: 172 or (b) a nucleotide sequence having at least 95% sequence identity to SEQ ID NO: 173. In some embodiments, the ERE comprises (a) a nucleotide sequence having at least 98% sequence identity to SEQ ID NO: 171 and a nucleotide sequence having at least 98% sequence identity to SEQ ID NO: 172 or (b) a nucleotide sequence having at least 98% sequence identity to SEQ ID NO:173. In some embodiments, the ERE comprises (a) a nucleotide sequence having at least 99% sequence identity to SEQ ID NO: 171 and a nucleotide sequence having at least 99% sequence identity to SEQ ID NO:172 or (b) a nucleotide sequence having at least 99% sequence identity to SEQ ID NO: 173. In some embodiments, the ERE comprises (a) a nucleotide sequence having 100% sequence identity to SEQ ID NO: 171 and a nucleotide sequence having 100% sequence identity to SEQ ID NO: 172 or (b) a nucleotide sequence having 100% sequence identity to SEQ ID NO: 173.

In some embodiments, the rAAV further comprises a chimeric intron formed from intron sequences derived from chicken beta actin and/or human betaherpes virus and/or human beta globin and/or operably linked to the MTM1 coding sequence.

In some embodiments, the chimeric intron comprises a nucleotide sequence derived from human beta globin, which optionally comprises a nucleotide sequence having at least 90% sequence identity to SEQ ID NO: 174. In some embodiments, the chimeric intron comprises a nucleotide sequence derived from human beta globin comprises SEQ ID NO: 174.

In some embodiments, the chimeric intron comprises a nucleotide sequence derived from human beta herpes virus, which optionally comprises a nucleotide sequence having at least 90% sequence identity to SEQ ID NO: 175. In some embodiments, the nucleotide sequence is derived from human human beta herpes virus comprises SEQ ID NO: 175.

In some embodiments, the chimeric intron is formed from introns from human beta herpes virus and rabbit beta globin. In some embodiments, the chimeric intron comprises a nucleotide sequence having at least 90% sequence identity to SEQ ID NO: 176. In some embodiments, the chimeric intron comprises a nucleotide sequence having at least 95% sequence identity to SEQ ID NO: 176. In some embodiments, the chimeric intron comprises a nucleotide sequence having at least 98% sequence identity to SEQ ID NO: 176. In some embodiments, the chimeric intron comprises a nucleotide sequence having at least 99% sequence identity to SEQ ID NO: 176. In some embodiments, the chimeric intron comprises a nucleotide sequence having 100% sequence identity to SEQ ID NO: 176. In some embodiments, the chimeric intron comprises the nucleotide sequence of SEQ ID NO: 176.

In some embodiments, the rAAV comprises an unmodified or modified AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu.9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc80-55; Anc80-129; Anc80-156; Anc80-751; Anc80-1029; Anc80-1712; Anc110; or Anc80DI capsid protein.

In some embodiments, the rAAV comprises an unmodified or modified rAAV9 capsid protein. In some embodiments, the rAAV comprises a VP1, VP2 and/or VP3 capsid protein comprising an amino acid sequence having at least 90% sequence identity to the corresponding protein(s) in AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu.9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc80-55; Anc80-129; Anc80-156; Anc80-751; Anc80-1029; Anc80-1712; Anc110; or Anc80DI.

In some embodiments, the rAAV comprises a VP1, VP2 and/or VP3 capsid protein comprising an amino acid sequence having at least 95% sequence identity to the corresponding protein(s) in AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu.9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D, rh.62-D, AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc80-55; Anc80-129; Anc80-156; Anc80-751; Anc80-1029; Anc80-1712; Anc110; or Anc80DI.

In some embodiments, the rAAV comprises a VP1, VP2 and/or VP3 capsid protein comprising an amino acid sequence having at least 98% sequence identity to the corresponding protein(s) in AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B, rh.24-B; rh.64-B; hu.27-B; hu.21-B; hu.22-B; hu.23-B; hu.7-C; hu.61-C; rh.56-C; hu.9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74, hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1, Anc80-55; Anc80-129, Anc80-156; Anc80-751; Anc80-1029; Anc80-1712; Anc110; or Anc80DI.

In some embodiments, the rAAV comprises a VP1, VP2 and/or VP3 capsid protein comprising an amino acid sequence having at least 99% sequence identity to the corresponding protein(s) in AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu.9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh.74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc80-55; Anc80-129; Anc80-156; Anc80-751; Anc80-1029; Anc80-1712; Anc110; or Anc80DI.

In some embodiments, the rAAV comprises a VP1, VP2 and/or VP3 capsid protein comprising an amino acid sequence having 100% sequence identity to the corresponding protein(s) in AAV2; AAV1; AAV6; AAV3; AAV LK03; AAV7, AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu.9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc80-55; Anc80-129; Anc80-156; Anc80-751; Anc80-1029; Anc80-1712; Anc110; or Anc80DI.

In some embodiments, the rAAV comprises a modified AAV capsid protein comprising at least one liver-toggle mutation as compared to a reference capsid protein.

In some embodiments, the reference capsid protein is a VP1, VP2 and/or VP3 protein. In some embodiments, the reference AAV capsid protein is a capsid protein having any one of SEQ ID NOs: 54-152 or a fragment thereof.

In some embodiments, the at least one liver-toggle mutation comprises: an alanine (A) or glycine (G) amino acid residue at an amino acid position corresponding to position 266 in Anc80; and/or a lysine (K) or arginine (R) amino acid residue at an amino acid position corresponding to position 168 in Anc80.

In some embodiments, the at least one liver-toggle mutation comprises: an alanine (A) amino acid residue at an amino acid position corresponding to position 266 in Anc80; and/or a lysine (K) amino acid residue at an amino acid position corresponding to position 168 in Anc80.

In some embodiments, the at least one liver-toggle mutation comprises: an alanine (A) amino acid residue at an amino acid position corresponding to position 266 in Anc80; and/or an arginine (R) amino acid residue at an amino acid position corresponding to position 168 in Anc80.

In some embodiments, the at least one liver-toggle mutation comprises: a glycine (G) amino acid residue at an amino acid position corresponding to position 266 in Anc80; and/or a lysine (K) amino acid residue at an amino acid position corresponding to position 168 in Anc80.

In some embodiments, the at least one liver-toggle mutation comprises: a glycine (G) amino acid residue at an amino acid position corresponding to position 266 in Anc80; and/or an arginine (R) amino acid residue at an amino acid position corresponding to position 168 in Anc80.

In some embodiments, the at least one liver-toggle mutation comprises an alanine (A) at an amino acid position corresponding to position 267 in AAV9. In some embodiments, the at least one liver-toggle mutation comprises a threonine (T) at an amino acid position corresponding to position 269 in AAV9.

In some embodiments, the capsid protein is a modified AAV9 capsid protein, optionally wherein the capsid protein is a modified AAV9 VP1 capsid protein.

In some embodiments, the liver-toggle mutation comprises: an alanine (A) amino acid residue at an amino acid position corresponding to position 267 in AAV9; and a threonine (T) amino acid residue at an amino acid position corresponding to position 269 in AAV9.

In some embodiments, the liver-toggle mutation further comprises an alanine (A) amino acid residue at an amino acid position corresponding to position 504 in AAV9; and/or an Alanine (A) amino acid residue at an amino acid position corresponding to position 505 in AAV9.

In some embodiments, the liver-toggle mutant comprises the sequence NSTSGASS (SEQ ID NO: 160), NSTSGGST (SEQ ID NO: 161) or NSTSGAST (SEQ ID NO: 162). In some embodiments, the rAAV capsid protein has the sequence of SEQ ID NO: 159. In some embodiments, the rAAV capsid protein has the sequence of SEQ ID NO: 163.

In some embodiments, the one or more liver toggle mutations comprise one or more amino acid substitutions at one or more of Q263, S264. G265, A266, S267, N268, H271, N382. G383, S384, Q385, S446, R471, W502, T503, D528. D529, Q589, K706, and V708 as compared to an AAV2 reference capsid protein (SEQ ID NO:1 of WO2021/050614, which is incorporated by reference herein).

In some embodiments, the one or more liver toggle mutations comprise the amino acid substitution S446R as compared to a reference capsid protein. In some embodiments, the one or more liver toggle mutations comprise the amino acid substitution R471A as compared to a reference capsid protein. In some embodiments, the one or more liver toggle mutations comprise the amino acid substitution V708T or V708A as compared to a reference capsid protein.

In some embodiments, the rAAV comprises a modified AAV capsid protein comprising at least one muscle-targeting element as compared to a reference capsid protein. In some embodiments, the reference capsid protein is a VP1, VP2 and/or VP3 protein.

In some embodiments, the muscle targeting element is 7-mer peptide having the sequence RGDX₁X₂X₃X₄ (SEQ ID NO:52), wherein X₁ to X₄ are independently selected amino acid residues. In some embodiments, X₁, X₂, and X₃ are independently selected from L, G, V, and A; and X₄ is selected from S, V, A, G, and L. In some embodiments, X₁, X₂, and X₃ are independently selected from L, V, and A; and at least two of X₁, X₂, and X₃ are independently L. In some embodiments, X₂ is L.

In some embodiments, the muscle targeting element is 7-mer peptide having the sequence RGDX₁X₂X₃X₄ (SEQ ID NO:52), wherein X₁, X₂, X₃ and X₄ are independently selected from A, D, E, F, G, H, I, K, L, N, Q, R, S, T, V, and Y. In some embodiments, the 7-mer has a sequence selected from SEQ ID NOs.: 238-44858. In some embodiments, the 7-mer has a sequence selected from SEQ ID NOs.: 238-338. In some embodiments, the 7-mer has a sequence selected from SEQ ID NOs.: 238-438 In some embodiments, the 7-mer has a sequence selected from SEQ ID NOs.: 238-538. In some embodiments, the 7-mer has a sequence selected from SEQ ID NOs.: 238-638. In some embodiments, the 7-mer has a sequence selected from SEQ ID NOs.: 238-738. In some embodiments, the 7-mer has a sequence selected from SEQ ID NOs.: 238-838. In some embodiments, the 7-mer has a sequence selected from SEQ ID NOs.: 238-938. In some embodiments, the 7-mer has a sequence selected from SEQ ID NOs.: 238-1038.

In some embodiments, 7-mer peptide has a sequence of RGDLLLS (SEQ ID NO: 1). In some embodiments, the targeting peptide is the 7-mer peptide TLAVPFK (SEQ ID NO: 53). In some embodiments, the targeting peptide is a peptide having any one of SEQ ID NOs: 2-51 and 53.

In some embodiments, the muscle-targeting element consists of a 7-mer peptide having the sequence selected from SEQ ID NOs.: 238-44858 is inserted into a site within VR VIII of the AAV capsid protein. In some embodiments, the muscle-targeting element consists of a 7-mer peptide having the sequence RGDLLLS (SEQ ID NO: 1) inserted into a site within VR VIII of the AAV capsid protein. In some embodiments, the 7-mer peptide is inserted into an amino acid position between 565 and 595 of the reference AAV capsid protein.

In some embodiments, the reference AAV capsid protein is a capsid protein of AAV1 and a 7-mer muscle-targeting peptide is inserted between D590 and P591 or between S588 and T589 of the capsid protein; the reference AAV capsid protein is a capsid protein of AAV2 and the 7-mer muscle-targeting peptide is inserted between R588 and Q589 or between N587 and R588 of the capsid protein; the reference AAV capsid protein is a capsid protein of AAV3b and the 7-mer muscle-targeting peptide is inserted between S586 and S587 or between N588 and T589 of the capsid protein; the reference AAV capsid protein is a capsid protein of AAV4 and the 7-mer muscle-targeting peptide is inserted between S584 and N585 or between S586 and N587 of the capsid protein; the reference AAV capsid protein is a capsid protein of AAV5 and the 7-mer muscle-targeting peptide is inserted between S575 and S576 or between T577 and T578 of the capsid protein; the reference AAV capsid protein is a capsid protein of AA V6 and the 7-mer muscle-targeting peptide is inserted between D590 and P591 or S588 and T589 of the capsid protein; the reference AAV capsid protein is a capsid protein of AAV7 and the 7-mer muscle-targeting peptide is inserted between N589 and T590 of the capsid protein; the reference AAV capsid protein is a capsid protein of AAV8 and the 7-mer muscle-targeting peptide is inserted between N590 and T591 of the capsid protein; the reference AAV capsid protein is a capsid protein of AAV9 and the 7-mer muscle-targeting peptide is inserted between Q588 and A589 of the capsid protein; the reference AAV capsid protein is a capsid protein of AAVrh10 and the 7-mer muscle-targeting peptide is inserted between N590 and A591 of the capsid protein; the reference AAV capsid protein is a capsid protein of AAVpo.1 and the 7-mer muscle-targeting peptide is inserted between N567 and S568 or between N569 and T570 of the capsid protein; or the reference AAV capsid protein is a capsid protein of AAV12 and the 7-mer muscle-targeting peptide is inserted between N592 and A593 or between T594 and T595 of the capsid protein.

In some embodiments, the muscle targeting peptide is inserted into a site within VR VIII of a liver-toggle mutant capsid, optionally a liver-toggle mutant capsid as described in any one of embodiments 49 to 62. In some embodiments, the muscle targeting peptide is inserted into an amino acid position between 565 and 595 of the liver toggle mutant.

In some embodiments, the reference AAV capsid protein is a capsid protein of AAV1 and the targeting peptide is inserted between D590 and P591 or between S588 and T589 of the liver-toggle mutant; the reference AAV capsid protein is a capsid protein of AAV2 and the targeting peptide is inserted between R588 and Q589 or between N587 and R588 of the liver-toggle mutant; the reference AAV capsid protein is a capsid protein of AAV3b and the targeting peptide is inserted between S586 and S587 or between N588 and T589 of the liver-toggle mutant; the reference AAV capsid protein is a capsid protein of AAV4 and the targeting peptide is inserted between S584 and N585 or between S586 and N587 of the liver-toggle mutant; the reference AAV capsid protein is a capsid protein of AAV5 and the targeting peptide is inserted between S575 and S576 or between T577 and T578 of the liver-toggle mutant; the reference AAV capsid protein is a capsid protein of AAV6 and the targeting peptide is inserted between D590 and P591 or S588 and TS89 of the liver-toggle mutant; the reference AAV capsid protein is a capsid protein of AAV7 and the targeting peptide is inserted between N589 and T590 of the liver-toggle mutant; the reference AAV capsid protein is a capsid protein of AAV8 and the targeting peptide is inserted between N590 and T591 of the liver-toggle mutant; the reference AAV capsid protein is a capsid protein of AAV9 and the targeting peptide is inserted between Q588 and A589 of the liver-toggle mutant; the reference AAV capsid protein is a capsid protein of AAVrh10 and the targeting peptide is inserted between N590 and A591 of the liver-toggle mutant; the reference AAV capsid protein is a capsid protein of AAVpo.1 and the targeting peptide is inserted between N567 and S568 or between N569 and T570 of the liver-toggle mutant; or the reference AAV capsid protein is a capsid protein of AAV12 and the targeting peptide is inserted between N592 and A593 or between T594 and T595 of the liver-toggle mutant.

In some embodiments, the capsid protein has the sequence of SEQ ID NO: 158. In some embodiments, the rAAV capsid protein has the sequence of SEQ ID NO: 159.

In some embodiments, the ERE comprises a constitutive promoter. In some embodiments, the constitutive promoter is the Rous sarcoma virus (RSV) LTR promoter (optionally with the RSV enhancer), the cytomegalovirus (CMV) promoter (optionally with the CMV enhancer), the SV40 promoter, the dihydrofolate reductase (DHFR) promoter, the β-actin promoter, the phosphoglycerol kinase 1 (PGK1) promoter (optionally the minimal PGK1 promoter), or the EF1 alpha promoter (optionally with intron).

In some embodiments, the ERE comprises an inducible promoter. In some embodiments, the inducible promoter is a tetracycline or rapamycin inducible promoter. In some embodiments, the ERE comprises a muscle-specific promoter. In some embodiments, the muscle specific promoter is a desmin promoter (which is optionally a CpG depleted desmin promoter), a CKM promoter derivative or an MTM1 promoter. In some embodiments, the promoter is a human promoter.

In some embodiments, the rAAV comprises a rabbit globin poly A sequence 3′ to the MTM1 coding sequence, optionally wherein the rabbit globin poly A sequence has at least 90% sequence identity to SEQ ID NO: 177. In some embodiments, the rabbit globin poly A sequence has at least 95% sequence identity to SEQ ID NO: 177. In some embodiments, the rabbit globin poly A sequence has at least 98% sequence identity to SEQ ID NO: 177. In some embodiments, the rabbit globin poly A sequence has at least 99% sequence identity to SEQ ID NO: 177. In some embodiments, the rabbit globin poly A sequence has 100% sequence identity to SEQ ID NO: 177.

In some embodiments, the genome of the rAAV comprises AAV-derived inverted terminal repeat sequences (ITRs). In some embodiments, the ITRs are derived from AAV serotype 2. In some embodiments, the rAAV comprises a first ITR having at least 90% sequence identity to SEQ ID NO: 178 and a second ITR having at least 90% sequence identity to SEQ ID NO: 179. In some embodiments, the first ITR has at least 95% sequence identity to SEQ ID NO: 178 and the second ITR has at least 95% sequence identity to SEQ ID NO: 179. In some embodiments, the first ITR has at least 98% sequence identity to SEQ ID NO: 178 and the second ITR has at least 98% sequence identity to SEQ ID NO:179. In some embodiments, the first ITR has at least 99% sequence identity to SEQ ID NO: 178 and the second ITR has at least 99% sequence identity to SEQ ID NO: 179. In some embodiments, the first ITR 100% sequence identity to SEQ ID NO: 178 and the second ITR has 100% sequence identity to SEQ ID NO: 179.

In some embodiments, the rAAV comprises a heterologous splice acceptor sequence 5′ to the MTM1 coding sequence. In some embodiments, the heterologous splice acceptor sequence is derived from human beta globin exon 3. In some embodiments, the heterologous splice acceptor sequence comprises the nucleotide sequence of SEQ ID NO: 180.

In one aspect, the present disclosure provides an rAAV comprising: modified AAV capsid protein comprising at least one liver-toggle mutation and/or one muscle-targeting element, optionally wherein the modified capsid protein comprises the amino acid sequence of SEQ ID NO:158, SEQ ID NO: 159, or SEQ ID NO: 163, and a genome comprising: a first ITR sequence; a hybrid expression regulatory element (ERE) comprising a CMV enhancer and a chicken beta actin promoter, optionally wherein the ERE comprises the nucleotide sequence of SEQ ID NO: 173; an MTM1 coding sequence operably linked to the ERE; and a second ITR sequence.

In some embodiments, the rAAV further comprises a chimeric intron between the ERE and the MTM1 coding sequence, optionally wherein the chimeric intron comprises the nucleotide sequence of SEQ ID NO: 176. In some embodiments, the rAAV further comprises a splice acceptor site 5′ to the MTM1 coding sequence, optionally wherein the splice acceptor site comprises the nucleotide sequence of SEQ ID NO:180. In some embodiments, the rAAV further comprises a polyadenylation sequence 3′ to the MTM1 coding sequence, optionally wherein the polyadenylation sequence comprises the nucleotide sequence of SEQ ID NO: 177.

In some embodiments, the MTM1 coding sequence is codon optimized for expression in human cells, optionally wherein the MTM1 coding sequence comprises the nucleotide sequence of SEQ ID NO: 167, SEQ ID NO:168, SEQ ID NO: 169 or SEQ ID NO: 170.

In some embodiments, the rAAV has a genome which is self-complementary, optionally wherein the genome is fully self-complementary.

The present disclosure further provides a pharmaceutical composition comprising the rAAV described herein and a pharmaceutically acceptable carrier. In some embodiments, the pharmaceutical composition is in the form of a unit dose.

In some embodiments, the pharmaceutical composition comprises 1×10¹⁰ to 1×10¹⁶ genome copy numbers (GC) of the rAAV and/or in which the rAAV concentration is 1×10¹⁰ vg/ml to 1×10¹⁶ vg/ml.

In some embodiments, the pharmaceutical composition is formulated for parenteral administration, for example systemic (e.g., intravenous), intramuscular or subcutaneous administration.

The present disclosure further discloses a host cell engineered to produce the rAAV described herein. In some embodiments, the host cell comprises a polynucleotide expressing one or more capsid proteins of the rAAV, a functional rep gene, and a recombinant nucleic acid vector comprising AAV ITRs and the MTM coding sequence operably linked to an expression regulatory element (ERE), optionally wherein the ERE is a hybrid ERE comprising a CMV enhancer and a chicken beta actin promoter.

In another aspect, the present disclosure provides a method for treating or ameliorating or preventing X-linked myotubular myopathy in a subject, comprising administering a therapeutically effective amount of the rAAV or the pharmaceutical composition described herein. In some embodiments, the effective dose comprises 1×10¹⁰ to 1×10¹⁶ genome copy numbers (GC) of the rAAV. In some embodiments, the effective dose is 1×10¹⁵ GC or less. In some embodiments, the effective dose is 5×10¹⁴ GC or less. In some embodiments, the effective dose is 1×10¹⁴ GC or less. In some embodiments, the effective dose is 5×10¹³ GC or less. In some embodiments, the effective dose is 1×10¹³ GC or less.

In some embodiments, the administration is parenteral. In some embodiments, the administration is systemic (e.g., intravenous). In some embodiments, the administration is intramuscular. In some embodiments, the administration is subcutaneous.

In yet another aspect, the present disclosure provides the rAAV or the pharmaceutical composition described herein for use in treating and/or preventing X-linked myotubular myopathy. In some embodiments, the rAAV or the pharmaceutical composition is for use in expressing myotubularin in a muscle cell.

Without being bound by theory, it is believed that the rAAVs of the disclosure have improved therapeutics indices due to higher MTM1 expression levels per viral genome administered and/or reduce off-target (e.g., liver) tropism or expression per viral genome administered as compared to a control rAAV whose genome comprises the MTM1 coding sequence under the control of the desmin promoter and/or includes an unmodified capsid protein.

### 5. BRIEF DESCRIPTION OF THE SEVERAL VIEWS OF THE DRAWINGS

These and other features, aspects, and advantages of the present invention will become better understood with regard to the following description, and accompanying drawings where:

FIG. 1 illustrates the structure of an AAV VP1 protein with certain variable regions (VR I, VR III, VR IV) highlighted. The location of the liver toggle (mut1) in VR I and the peptide insertion (deco1) in VR VIII are indicated.

FIGS. 2A-2C provide the sequence alignment of VP1 sequences of certain AAV variants using AAV2 VP1 as a reference. The location of residue 168, the liver toggle site, mut1 (FIG. 2A), and the insertion site of a targeting peptide (FIG. 2B), are indicated. FIGS. 2A-2B disclose SEQ ID NOs: 55, 54, 58, 56, 64, 59, 60, 89, 111, 61, 63, 62, and 57, respectively, in order of appearance.

FIGS. 3A-3D provide the sequence alignment of VP1 sequences of ancestral AAVs using AAV2 as a reference. The location of the liver toggle sites, residue 168 (FIG. 3A), and residue 266 (FIG. 3B), and the insertion site of a targeting peptide (FIG. 3C), are indicated. One or more representative member sequences for each of the Anc80. Anc81. Anc82, Anc83, Anc84, Anc94, Ac110, Anc113, Anc126 and Ane127 libraries were used for the alignment. FIGS. 3A-3D disclose SEQ ID NOs: 217-237, respectively, in order of appearance.

FIGS. 4A-4J shows immunohistochemistry data obtained from the experiment described in Example 2 below in the Example section. Anti-GFP immunohistochemistry was performed on liver with vehicle (FIG. 4A), AAV9 (FIG. 4B), AAV^{mut1} (FIG. 4C), AAV^{deco1} (FIG. 4D), or AAV^{mut1-deco1} (FIG. 4E); and skeletal muscle (quadriceps) tissue cross-sections of mice injected with vehicle (FIG. 4F), AAV9 (FIG. 4G), AAV^{mut1} (FIG. 4H), AAV^{deco1}) (FIG. 4I), or AAV^{mut1-deco1} (FIG. 4J).

FIGS. 5A-5B show mRNA expression in various tissues of C57BL/6 mice treated with different AAV vectors, as measure by RT-ddPCR. Y-axis represents the ratio of copies of eGFP mRNA transcripts over RPP30 mRNA and x-axis represents AAV vectors and the dose injected into the experimental animals. Each graph shows eGFP expression in liver (FIG. 5A) and quadriceps (FIG. 5B).

FIGS. 6A-6E show eGFP mRNA expression in various tissues of C57BL/6 mice treated with different AAV vectors, as measure by RT-ddPCR. Y-axis represents the ratio of copies of eGFP over RPP30 mRNA and x-axis represents AAV vectors and the dose injected into the experimental animals. Each graph shows eGFP expression in liver (FIG. 6A), heart (FIG. 6B), triceps surae (FIG. 6C), quadriceps (FIG. 6D), or diaphragm (FIG. 6E).

FIGS. 7A-7D show eGFP vector genome (DNA) and eGFP expression (mRNA) in liver and quad tissues of C57BL/6 mice treated with vehicle. AAV^{Mut1} and AAV^{Mut1-deco1} AAV vectors. DNA data is shown in FIGS. 7A and 7B with eGFP genomic copies as measured by RT-ddPCR plotted at 14 and 28 days, respectively. Y-axis represents vector genome (copies per DPG) and x-axis represents vehicle and AAV vectors. mRNA data is shown in FIGS. 7C and 7D with eGFP expression as measured by RT-ddPCR plotted at 14 and 28 days, respectively. Y-axis represents the ratio of copies of eGFP over RPP30 mRNA and x-axis represents AAV vectors.

FIG. 8 shows eGFP mRNA expression in various tissues of BalbC mice treated with vehicle, AAV^{mut1} and AAV^{mut1-deco1} AAV vectors, as measured by RT-ddPCR. Y-axis represents the ratio of copies of eGFP over RPP30 mRNA and x-axis represents AAV vectors and the dose injected into the experimental animals. The graph shows eGFP expression in liver (left) and quadriceps (right).

FIGS. 9A and 9B show exemplary IHC tissue analysis obtained from of Run 1 samples from NHPs. Liver tissue is shown in FIG. 9A, the left side shows tissue obtained from an AAV9 vector treated NHP and the right side shows tissue obtained from an AAV^{mut1_deco1} vector treated NHP; exemplary IHC quadriceps tissue is shown in FIG. 9B, obtained from AAV9 vector treated NHP on left and AAV^{mut1_deco1} vector treated NHP on the right.

FIG. 10 shows the % GFP positive cells in the liver tissue (right and left side of the organ) and quadriceps tissue (right and left leg) in slides obtained from Run 1 from NHPs administered vehicle, AAV9 or AAV^{mut1_deco1} vector.

FIG. 11 shows the % GFP positive cells in various skeletal muscle and liver tissue (average from Runs 1 and 2) in slides obtained from NHPs administered vehicle, AAV9 or AAV^{mut1_deco1} vector.

FIG. 12 shows the % GFP positive cells per animal in various skeletal muscle and liver tissue (average from Runs 1 and 2) in slides obtained from NHPs administered vehicle, AAV9 or AAV^{mut1_deco1} vector.

FIG. 13 shows the average combined quantification of % GFP positive cells per animal in various skeletal muscle and liver tissue (average from Runs 1 and 2) obtained from NHPs administered vehicle, AAV9 or AAV^{mut1_deco1} vector.

FIG. 14 shows the % GFP positive cells in various cardiac tissues (average from Runs 1 and 2) obtained from NHPs administered vehicle, AAV9 or AAV^{mut1_deco1} vector.

FIG. 15 shows the % GFP positive cells per animal in various cardiac muscle (average from Runs 1 and 2) obtained from NHPs administered vehicle, AAV9 or AAV^{mut1_deco1} vector.

FIG. 16 shows the average % GFP positive cells per animal in ventricle wall, atria, inter ventr septum slides (average from Runs 1 and 2) obtained from NHPs administered vehicle, AAV9 or AAV^{mut1_deco1} vectors.

FIGS. 17A-17C shows the average % GFP positive cells per NHP animal in various tissues (average from Runs 1 and 2) administered vehicle and AAV9 and AAV^{mut1_deco1} vectors. FIG. 17A shows average % GFP positive cells per animal in liver tissue. FIG. 17B shows average % GFP positive cells per animal in various skeletal muscle tissue. FIG. 17C shows average % GFP positive cells per animal in various cardiac tissue.

FIGS. 18A-18D show the results of DNA samples analyzed for biodistribution of vector genomes in the liver and quadriceps tissue using a duplexed ddPCR method targeting the transgene (eGFP) and a reference gene (RPP30). The results are shown in FIG. 18A (liver), FIG. 18B (quadriceps), FIG. 18C (biceps), and FIG. 18D (heart) where the x-axis represents AAV vectors (wild type AAV9 on the left and AAV^{mut1deco1} on the right of each plot) and indicating whether the sample was taken from the left or right side of the organ/animal.

FIGS. 19A-19D show the results of mRNA transcript analysis measured by eGFP copies of eGFP over RPP30 mRNA. FIG. 19A (liver), FIG. 19B (quadriceps), FIG. 19C (biceps), and FIG. 19D (heart), are illustrated where the x-axis represents AAV vectors (wild type AAV9 on the left and AAV^{mut1deco1} on the right) and indicating whether the sample was taken from the left or right side of the organ/animal.

FIG. 20 shows human MTM1 protein expression in RD cells. The expression level of human MTM protein was determined by automated JESS-ProteinSimple instrument. Each bar represents by peak area values of JESS, either before (blue) or after (orange) being normalized to total protein load. Data were obtained from one run using the 1:4 dilution as described in the western protocol.

FIG. 21 provides a study design to assess distribution patterns of Anc80 variants in non-human primates, as described in Example 8.

FIG. 22 provides a scatter plot of Anc80 variant counts from an Anc80 library showing each variant's enrichment in muscle (y-axis) and the liver (x-axis).

FIG. 23 shows negative log fold changes in tissues compared to the test article input (Anc80 variants of Anc80 library) on day 28 in liver-off variants (variants with “A” at P3) of Anc80 library (left) and liver-on variants (variants with “G” at P3) of Anc80 library (right). The test article, used herein, refers to the pool of vectors administered.

FIG. 24 shows the effect of P3 on log fold-change of tissue enrichment in primate G62N of Group 2 in day 28 quadriceps. A significant difference of Log fold-change of tissue enrichment in the quadriceps was found between “liver off” “0” and “liver on” “1” Anc80 variants. On the x-axis, “0” represents amino acid residue “G”, and “1.0” represents amino acid “A”.

FIG. 25 shows the effect of P3 on log fold-change of tissue enrichment in the quadriceps in primate 066E of Group 2 in day 28 quadriceps. A significant difference of log fold-change of tissue enrichment in the quadriceps was found between “liver off” “0” and “liver on” “I” Anc80 variants. On the x-axis, “0” represents amino acid residue “G”, and “1.0” represents amino acid “A”.

FIG. 26 shows the effect of P3 on average log fold-change of tissue enrichment in the quadriceps in both primates of Group 2 on day 28 quadriceps. A significant difference of log fold-change of tissue enrichment in the quadriceps was found between “liver off” “0” and “liver on” “1” Anc80 variants. On the x-axis, “0” represents amino acid residue “G”, and “1.0” represents amino acid “A”.

FIG. 27 shows a quadrant liver plot of G62N primate on day 28. The upper left (UL) quadrant bad higher biodistribution of 74 Anc80 variants in the quadricep muscle with liver toggle on “liver on”. The upper right (UR) quadrant had high biodistribution of 3 Anc80 variants in the quadricep muscle with liver toggle on and off. The lower left (LR) quadrant had low biodistribution of 56 Anc80 variants in the quadricep muscle with liver toggle on. The lower right (LL) quadrant bad higher distribution of 891 Anc80 variants in the quadricep muscle with liver toggle on “liver on” and off.

FIG. 28 shows a quadrant liver plot of G66E primate on day 28. The upper left (UL) quadrant had higher biodistribution of 66 Anc80 variants in the quadricep muscle with liver toggle on “liver on”. The upper right (UR) quadrant had high biodistribution of 125 Anc80 variants with liver toggle on and off. The lower left (LL) quadrant had low biodistribution of 271 Anc80 variants in the quadricep muscle with liver toggle on and off. The lower right (LR) quadrant had lower distribution of 566 Anc80 variants in the quadricep muscle with liver toggle on “liver on”.

FIG. 29 provides a flow chart representing comparative data amongst animals in each of Group 1 and Group 2. When the tissue enrichment data for 2 primates for each group was averaged, 13 variant pairs were found to have an effect on liver toggle. Variability in AAV transduction is common, particularly at low doses in primates. To use the limited data from two primates to evaluate the low doses of each variant in the ANc80 library, the average fold-enrichment of each variant was used rather than rely solely on enrichments from each animal.

FIG. 30 shows an average of the liver toggle data between both non-primate animals G62N and G66E of Group 2. The upper left (UL) quadrant had higher biodistribution of 69 Anc80 variants in the quadricep muscle with liver toggle on “liver on” The upper right (UR) quadrant had high biodistribution of 13 Anc80 variants in the quadricep muscle with liver toggle on and off. The lower left (LL) quadrant had low biodistribution of 687 Anc80 variants in the quadricep muscle with liver toggle on and off. The lower right (LR) quadrant had lower distribution of 257 Anc80 variants in the quadricep muscle with liver toggle on “liver on”.

FIG. 31 shows a fingerprint plot for the top 100 Anc80 variant positions P1 (amino acid 168 of Anc80 capsid), P2 (amino acid 205), P3 (amino acid 266), P4, . . . . P10 (amino acid 587), P11 (amino acid 609) on Anc80 scaffold. The Anc80 variants are color coded by the toggle amino acid at that particular position (P1-P11). “0” represents the color green, while 1 represents the color red. Variants having different amino acids at the variation positions are color coded. Variants were sorted and ranked in decreasing order by liver on fold-change of tissue enrichment in quadricep muscle. As shown, position P3 contains amino acid G in red for all 100 Anc80 variants.

FIG. 32 shows linear modeling of a position pattern evaluation on average log fold-change of P1-P11 variant pairs. The position pattern evaluation provides tissue enrichment scores versus average log fold changes at every amino acid position (P1-P11) The analysis shows that variations at P3, P5, P6 and P10 have some pattern impact on the average log fold changes (log (FC)) to the quadricep muscle.

FIG. 33 provides binary codes described in FIG. 31 for liver toggle variant pairs in the Anc80 library of Example 8. P3 represents liver toggle variant pairs of “liver off” and “liver on”. As shown in the bottom left scatter plot, some Anc80 variants are more present in muscle only with “liver off” (LR) which was dependent on liver toggle, while other variants worked well in both states “liver on” and “liver off” (UR).

FIG. 34 provides amino acid positions of 50,625 RGD targeting peptides in the RGD targeting peptide library of as described in Table 27. “Y1, Y2, Y3. Y4. Y5, Y6, and Y7”. Y1 denotes amino acid residue “R”, Y2 denotes amino acid residue “G”, and Y3 denotes amino acid residue “D”, in X₁X₂X₃RGDX₇X₈X₉X₁₀ described in the present disclosure. Y4 is denoted as “X₇” of X₁X₂X₃RGDX₇X₈X₉X₁₀, Y5 is denoted as “X₈” of X₁X₂X₃RGDX₇X₈X₉X₁₀, Y6 is denoted as X₉” of X₁X₂X₃RGDX₇X₈X₉X₁₀, and Y7 is denoted as “X₁₀” of X₁X₂X₃RGDX₇X₈X₉X₁₀. Example amino acid modifications of Y4, Y5, Y6, and Y7 regions of the targeting peptide include those described herein.

FIG. 35 shows a density plot of the density of vector DNA at a specific enrichment score ((Log 2 (Fold)) for AAV9 capsids having specific amino acids in position 4 (“Y4”, or “X₇” in X₁X₂X₃RGDX₇X₈X₉X₁₀), position 5 (“Y5”, or “X₈” in SEQ ID NO: 44915), position 6 (“Y6”, or “X₉” in X₁X₂X₃RGDX₇X₈X₉X₁₀) or position 7 (“Y7”, or “X₁₀” in X₁X₂X₃RGDX₇X₈X₉X₁₀) within the targeting peptide sequence. The various colors represent the amino acid at the particular position.

FIG. 36A shows the muscle-tropic sequence motif identified from the experiments described in Example 11. FIG. 36B is a scatter plot showing muscle enrichment through distribution of scaled enrichment scores of variants tested in Example 11 (an inverse coefficient of variation (ICV)). The red dots on the scatter plot represent variants with a muscle-tropic sequence motif. The mean-ICV plot shows the relationship between mean enrichment score and inverse CV (ICV) of the enrichment score for each variant in the library. The ICV plot is useful for selecting variants with high average tissue enrichment score and low variability (high ICV). FIG. 36C compares distribution scaled enrichment scores of variants with and without the muscle-tropic motif. The cumulative density of variants is shown at specific tissue enrichment scores (Log 2 (fold)). The lines are colored by the probability of amino acids at a particular position.

FIG. 37A shows a sequence motif identified from top 10 myotropic variants as described in Table 27 that was shown to have significant sequence similarity. FIG. 37B is a scatter plot showing muscle enrichment scores of variants (an inverse coefficient of variation (ICV)) where the top muscle targeting peptides are in red. The mean-ICV plot shows the relationship between mean enrichment score and inverse CV (ICV) of the enrichment score for each variant in the library. The ICV plot is useful for selecting variants with high average tissue enrichment score and low variability (high ICV). FIG. 37C is a network plot where the lines connect dots representing variants with similar sequences. 8 out of 10 top variants were connected in a sequence similarity network. FIG. 37C shows the sequence similarity network of SEQ ID NO: 238 (“ATLVT013XX38181”), the top targeting peptide for muscle tropism.

FIG. 37D shows a sequence motif enriched in muscle tropic capsids.

FIG. 37E shows a network comparison of the targeting peptides described herein to literature myopeptides.

FIG. 38A provides a network plot and FIG. 38B provides a scatter plot showing tissue enrichment scores for each tissue region assessed. The network analysis and scatter plot show that SEQ ID NO: 238 (“ATLVT013XX38181”) outperformed wild-type AAV9. AAV9^{deco1} (AAV9 capsid with the deco1 peptide), and AAV9^{myo3E} (AAV9 capsid with a myo3E peptide) capsids for muscle tropism, and reduces liver toxicity due to the liver-detargeting phenotype Mut1 as compared to wild-type AAV9. The targeting peptide SEQ ID NO: 238 (“ATLVT013XX38181”) consistently ranked at the top for target enrichment in all tissues except for the triceps.

FIG. 38C shows a plot of tissue enrichment scores (log 10 scale of expression) for liver and the indicated muscle tissues. Capsids tested included AAV9 and AAV9 comprising an M3 (MYODV6 (SEQ ID NO: 44864)) targeting peptide located in VR VIII or an M2 (RGDRSVV (SEQ ID NO: 239)) targeting peptide located in VR VIII between amino acids at positions 588 and 589. Muscle tissues analyzed included ventricle wall of the heart, biceps femoris, diaphragm, tibialis anterior, and triceps brachii Higher tissue enrichment score values represent greater tropism.

FIG. 39 shows the probability of the top 100 targeting peptides at positions Y4, Y5, Y6 and Y7, where amino acid “R” at position 4 (Y4) is enriched, and amino acids “V, I, and L” are enriched at position 7 (Y7).

FIG. 40 shows a comparison of Anc80 variants with and without: a deco1 (SEQ ID NO: 1) targeting peptide inserted into the VR VIII region of the Anc80 variant capsid. 6 out of 7 Anc80 variants with a deco1 insertion at VR VIII enhanced muscle delivery of Anc80 variants.

FIG. 41, the present inventors created additional targeting peptide variants for insertion, substitution, or modification within the VR8 region of an AAV capsid by modifying the 3 amino acids preceding amino acids “RGD” in the targeting peptide having an amino acid sequence of SEQ ID NO: 44910 (termed “triplet”) and the 4 amino acids after “RGD” in the targeting peptide having an amino acid sequence of SEQ ID NO: 44910 peptide (termed “quad” or “quadruplet”). Additionally, the present inventors created additional targeting peptides for insertion, substitution, or modification within the VR8 region of an AAV capsid by modifying the 3 amino acids preceding amino acids “RGD” in the targeting peptide having an amino acid sequence of SEQ ID NO: 44880 and the 4 amino acids after the amino acids “RGD” in the targeting peptide having an amino acid sequence of SEQ ID NO: 44880.

FIG. 41 shows a list of 20 targeting peptide variants (myoDV1-myoDV10 and myoCD5-myoCD14). FIG. 41 shows tissue enrichment scores for each of the 20 targeting peptide variants in muscle tissue (diaphragm, flexor digitorum profundus, heart left ventricle wall, tibialis anterior, and triceps brachii). Higher tissue enrichment score values represent greater muscle tropism, with MYODV6 and MYODV8 having the highest averages tissue enrichment score for all muscle tissue regions.

FIG. 42 shows a network plot showing log 2 fold change (tissue enrichment score) for each of the muscle tissues analyzed for SEQ ID NO: 44880, MYODV6 (SEQ ID NO: 44864), MYODV8 (SEQ ID NO: 44866). MYOCD10 (SEQ ID NO: 44874), and MYOCD12 (SEQ ID NO: 44876) targeting peptides. Surprisingly, as shown in the network plot, MYODV6 and MYODV8 each have the highest overall log 2 fold change of tissue enrichment in each muscle tissue compared to MYOCD10 and MYCD12 (which contain amino acid residues “SNR” (triplet) preceding “RGD” within the targeting peptide. Additionally, MYODV6 and MYODV8 each have a higher overall log 2 fold change of tissue enrichment in each muscle tissue compared to the targeting peptide having an amino acid sequence of SEQ ID NO: 44880.

FIG. 43 shows a plot of tissue enrichment scores (log 10 scale of expression) for liver and the indicated muscle tissues. Capsids tested included AAV9 and AAV9 comprising an H1 (RGDLIGR (SEQ ID NO: 1422)) targeting peptide located in VR VIII between amino acids at positions 588 and 589 or an H2 (RGDQSTL (SEQ ID NO: 3052)) targeting peptide located in VR VIII between amino acids at positions 588 and 589. Muscle tissues analyzed included biceps femoris, diaphragm, tibialis anterior, triceps brachii, and ventricle wall of the heart. Higher tissue enrichment score values represent greater tropism.

FIG. 44 shows a plot of tissue enrichment scores (log 10 scale of expression) for liver and the indicated muscle tissues. Capsids tested included AAV9 and AAV9 comprising an S1 (RGDISRT (SEQ ID NO. 263)) targeting peptide located in VR VIII between amino acids at positions 588 and 589 or an S2 (RGDRSQT (SEQ ID NO: 251)) targeting peptide located in VR VIII between amino acids at positions 588 and 589. Muscle tissues analyzed included ventricle wall of the heart, biceps femoris, diaphragm, tibialis anterior, and triceps brachii. Higher tissue enrichment score values represent greater tropism.

FIG. 45 shows a plot for median percent GFP⁺ cells for liver and the indicated muscle tissues. Capsids tested included AAV9 and AAV9 comprising an M3 (“DV6” ENRRGDFNNL (SEQ ID NO: 44864)) targeting peptide located in VR VIII (M3 targeting peptide was inserted between 585 and 589 such that amino acids at positions 586, 587 and 588 were replaced with three amino acids from the targeting peptide); an M1 (“38181” SAQRGDRGQI (SEQ ID NO: 44911)) targeting peptide located in VR VIII (M1 targeting peptide was inserted between 585 and 589 such that amino acids at positions 586, 587 and 588 were replaced with three amino acids from the targeting peptide), and a mut1 VR I substitution, or an M1 (“38181” SAQRGDRGQI (SEQ ID NO: 44911)) targeting peptide located in VR VIII (M1 targeting peptide was inserted between 585 and 589 such that amino acids at positions 586, 587 and 588 were replaced with three amino acids from the targeting peptide) with a wild type VR I. Higher percent GFP⁺ cells represent greater tropism.

FIG. 46 shows representative IHC images for anti-GFP staining for the indicated tissues (quads, heart, and liver) for various AAV capsid proteins (AAV9. AAV9 comprising SEQ ID NO: 44880, AAV9 comprising an M3 targeting peptide (SEQ ID NO: 48391), and an AAV9 comprising an M1 targeting peptide (SEQ ID NO: 44864). Images were taken at 10× magnification. The IHC images in FIG. 46 served, in part, as the basis for the quantification in FIG. 45.

FIG. 47 shows a plot for ddPCR data for cells for liver and the indicated muscle tissues. Capsids tested included AAV9 and AAV9 comprising an M3 (“DV6” ENRRGDFNNL (SEQ ID NO: 44864)) targeting peptide located in VR VIII (M3 targeting peptide was inserted between 585 and 589 such that amino acids at positions 586, 587 and 588 were replaced with three amino acids from the targeting peptide); an M1 (“38181” SAQRGDRGQI (SEQ ID NO: 44911)) targeting peptide located in VR VIII (M1 targeting peptide was inserted between 585 and 589 such that amino acids at positions 586, 587 and 588 were replaced with three amino acids from the targeting peptide) and a mut1 VR I substitution, or an M1 (“38181” SAQRGDRGQI (SEQ ID NO: 44911)) targeting peptide located in VR VIII (M1 targeting peptide was inserted between 585 and 589 such that amino acids at positions 586, 587 and 588 were replaced with three amino acids from the targeting peptide) with a wild type VR I.

FIG. 48 provides a summary of eGFP mRNA expression in various tissues of C57BL/6 mice treated with different AAV vectors, as measured by RT-ddPCR and eGFP vector genomes copies per DPG (DNA) in various tissues of C57BL/6 mice treated with different AAV vectors, as measure by RT-ddPCR.

FIG. 49A provides a peptide segment within variable region I (VRI) of the AAV9 capsid protein, various amino acid positions (P₁, P₂, P₃, P₄, P₅, P₆, P₇, P₈, P₉, P₁₀, P₁₁, P₁₂) within the peptide segment and modifications made to the various positions for capsids present in the AAV^{-Lib1} capsid library.

FIG. 49B provides a sequence alignment of variable region I in certain AAV variants using AAV2 variable region I as a reference for numbering.

FIG. 50 shows vector genome (DNA) in liver of C57BL/6 mice treated with the AAV^{-Lib1} Library. Log 2Fold DNA levels are presented for each of the 3,456 capsid variants (variant ID on x-axis) in addition to AAV9 and AAV^{mut1}. Log 2Fold DNA levels of AAV9 and AAV^{mut1} are identified specifically in FIG. 50.

FIG. 51A is a schematic showing the amino acid residues in VRI with a box highlighting position P6, including the two amino acid residues at P6 for the AAV^{-Lib1} library: alanine (A) and glycine (G).

FIG. 51B shows a density plot of the amount of vector DNA in the liver (Log 2 (Fold)) for the sum of all capsids in AAV^{-Lib1} that include an alanine (A) at P6 (amino acid position 266) and a density plot of the amount of vector DNA in the liver (Log 2 (Fold)) for the sum of all capsids in AAV^{-Lib1} that include a glycine (G) at P6 (amino acid position 266).

FIG. 51C is an empirical cumulative distribution function (eCDF) plot showing the amount of vector DNA in the liver (Log 2 (Fold)) for the sum of all capsids in AAV^{-Lib1} that include an alanine (A) at P6 (amino acid position 266) and the amount of vector DNA in the liver (Log 2 (Fold)) for the sum of all capsids in AAV^{-Lib1} that include a glycine (G) at P6 (amino acid position 266).

FIG. 52A is a schematic showing the amino acid residues in VRI with boxes highlighting positions of interest: P₁, P₃, P₅, P₆, P₈, and P₁₂. In addition, FIG. 52A provides an example formula for calculating an enrichment score for the modified capsid proteins in the AAV^{-Lib1} library.

FIG. 52B is an empirical cumulative distribution function (eCDF) plot showing the amount of vector DNA in the liver (Log 2 (Fold)) for the sum of all capsids in the AAV^{-Lib1} library that include cither an alanine (A), a glutamate (E), a glutamine (Q), and a threonine (T) at P₃ (amino acid position 263).

FIG. 52C is an empirical cumulative distribution function (eCDF) plot showing the amount of vector DNA in the liver (Log 2 (Fold)) for the sum of all capsids in the AAV^{-Lib1} library that include either an alanine (A) or a glycine (G) at P₅ (amino acid position 265).

FIG. 52D is an empirical cumulative distribution function (eCDF) plot showing the amount of vector DNA in the liver (Log 2 (Fold)) for the sum of all capsids in the AAV^{-Lib1} library that include either an alanine (A) or a glycine (G) at P⁶ (amino acid position 266).

FIG. 53A is an enrichment plot showing the amount of vector DNA in the liver for the sum of all the capsids the AAV^{-Lib1} library that include either an alanine (A) or a glycine (G) at P⁶ (amino acid position 266).

FIG. 53B is an enrichment plot showing the amount of vector DNA in the liver for the sum of all the capsids the AAV-Lidl library having a P₅P₆ combination of AA, AG, GA, or GG.

FIG. 53C is an enrichment plot showing the amount of vector DNA in the liver for the sum of all the capsids in the AAV^{-Lib1} library having a P₃, P₅, and P₆ combination of AAA, AAG, AGA, AGG, EAA, EAG, EGA, EGG, QAA, QAG, QGA, QGG, TAA, TAG, TGA, and TGG (appearing from left to right on the x-axis).

FIG. 54 shows output of a network analysis of data from the AAV^{-Lib1} library Each circle indicates a capsid variant and a line connecting the capsids indicates sequence similarity.

FIG. 55 is an illustration of the structure of an AAV VP1 protein with positions P₃, P₅, P₆, and P₈ in VRI highlighted.

FIG. 56A is an empirical cumulative distribution function (eCDF) plot showing RNA expression in the liver (Log 2 (Fold)) for the sum of all capsids in the AAV^{-Lib1} library that include an alanine (A), a glutamate (E), a glutamine (Q), or a threonine (T) at P₃ (amino acid position 263).

FIG. 56B is an empirical cumulative distribution function (eCDF) plot showing RNA expression in the heart (Log 2 (Fold) for the sum of all capsids in the AAV^{-Lib1} library that include an alanine (A), a glutamate (E), a glutamine (Q), or a threonine (T) at P3 (amino acid position 263).

FIG. 56C is an empirical cumulative distribution function (eCDF) plot showing RNA expression in the liver (Log 2 (Fold)) for the sum of all capsids in the AAV^{-Lib1} library that include either an alanine (A) or a glycine (G) at P₅ (amino acid position 265).

FIG. 56D is an empirical cumulative distribution function (eCDF) plot showing the RNA expression in the heart (Log 2 (Fold)) for the sum of all capsids in the AAV^{-Lib1} library that include either an alanine (A) or a glycine (G) at P⁵ (amino acid position 265).

FIG. 56E is an empirical cumulative distribution function (eCDF) plot showing RNA expression in the liver (Log 2 (Fold)) for the sum of all capsids in the AAV^{-Lib1} library that include either an alanine (A) or a glycine (G) at P⁵ (amino acid position 266).

FIG. 56F is an empirical cumulative distribution function (eCDF) plot showing RNA expression in the heart (Log 2 (Fold) for the sum of all capsids in the AAV^{-Lib1} library that include either an alanine (A) or a glycine (G) at P₆ (amino acid position 266).

FIG. 57A shows enrichment in liver DNA (x-axis) plotted against enrichment in liver RNA (y-axis) for capsids comprising various combination of amino acid modifications at P₃, P₅, and P₆. Positions of AAV^{mut1} and AAV9 controls are indicated by arrows and corresponding text.

FIG. 57B shows enrichment in liver DNA (x-axis) plotted against enrichment in heart RNA (y-axis) for capsids comprising various combination of amino acid modification at P₃, P₅, and P₆. Positions of AAV^{mut1} and AAV9 controls are indicated by arrows and corresponding text.

FIG. 58A shows enrichment in liver DNA (x-axis) plotted against enrichment in liver RNA (y-axis) for capsids having a threonine (T), a glycine (G), and a glycine (G) combination of amino acid modifications at P₃, P₅, and P₆, respectively. Positions of AAV^{mut1} and AAV9 controls are indicated by arrows and corresponding text.

FIG. 58B shows enrichment in liver DNA (x-axis) plotted against enrichment in heart RNA (y-axis) for capsids having a threonine (T), a glycine (G), and a glycine (G) combination of amino acid modifications at P₃, P₅, and P₆, respectively. Positions of AAV^{mut1} and AAV9 controls are indicated by arrows and corresponding text.

FIG. 58C shows enrichment in liver DNA (x-axis) plotted against enrichment in heart RNA (y-axis) for capsids having a threonine (T), a glycine (G), and a glycine (G) combination of amino acid modifications at P₃, P₅, and P₆, respectively. Sub-populations of the capsids having a threonine (T) (yellow dots) or an asparagine (N)/a serine (S) (blue dots) at position P₁ are color-coded. Positions of AAV^{mut1} and AAV9 controls are indicated by arrows and corresponding text.

FIG. 58D shows enrichment in liver RNA (x-axis) plotted against enrichment in heart RNA (y-axis) for capsids having a threonine (T), a glycine (G), and a glycine (G) combination of amino acid residues at P₃, P₅, and P₆, respectively. Each dot represent a capsid having TGG at P₃, P₅, and P₆. Capsids having a particular amino acid residue (or combinations of amino acid residues) at positions P₁, P₂, PA, and P₁₂ (e.g., an asparagine (N), a glycine (G), a threonine (T), a serine (S), and/or an alanine (A)) are identified and shaded accordingly. Particular subpopulations having NGTT, NSTT, SGAA, and SGAT at positions P₁, P₂, P₄, and P₁₂ are identified. Positions of AAV^{mut1} and AAV9 controls are indicated by arrows and corresponding text.

FIG. 58E shows enrichment in liver RNA (x-axis) plotted against enrichment in heart RNA (y-axis) for capsids having a glutamine (Q), a glycine (G), and a glycine (G) combination of amino acid residues at P₃, P₅, and P₆, respectively. Each dot represent a capsid having TGG at P₃, P₅, and P₆. A subpopulation having a serine (S), a glycine (G), a threonine, and a histidine (H) at positions at positions P₁, P₂, P₄, and P₁₂ is identified. Positions of AAV^{mut1} and AAV9 controls are indicated by arrows and corresponding text.

FIG. 58F shows average enrichment in liver RNA (x-axis) plotted against enrichment in heart RNA (y-axis) for all AAV^{-Lib1} capsids having a threonine (T), a glycine (G), and a glycine (G) combination of amino acid residues at P₃, P₅, and P₆, respectively. Positions of AAV^{mut1} and AAV9 controls are indicated by corresponding text.

FIG. 58G shows enrichment in liver RNA (x-axis) plotted against enrichment in heart RNA (y-axis) for 24 selected capsids having a threonine (T), a glycine (G), and a glycine (G) combination of amino acid residues at P₃, P⁵, and P⁶, respectively. The 24 selected capsids correspond to groups: 1 NSTSGGP₇P₈NDNH (e.g., SEQ ID NO: 44927); group 2: NSTTGGP₇P₈NDNH (SEQ ID NO: 44928); group 4: SGTAGGP₇P₈NDNT (SEQ ID NO: 44930); group 5: SGTSGGP₇P₈NDNA (SEQ ID NO: 44931); group 6: SGTTGGP₇P₈NDNT (SEQ ID NO: 44932); and group 7: SSTAGGP₇P₈NDNA (SEQ ID NO: 44933). Positions of AAV^{mut1} and AAV9 controls are indicated by corresponding text.

FIGS. 58H-58I shows enrichment in liver RNA (x-axis) plotted against enrichment in heart RNA (y-axis) for 6 groups where each group has a threonine (T), a glycine (G), and a glycine (G) combination of amino acid residues at P₃, P₅, and P₆, respectively. FIG. 58H includes dark circles that identify the VRI mini-Lib peptide segments in the key. FIG. 58I includes dark circles that identify the VRI mini-Lib peptide segments in the key.

FIG. 59 shows vector genome (DNA) in liver of NHP treated with the AAV^{-Lib1} Library. Log 2Fold DNA levels are presented for each of the 3,456 capsid variants (variant ID on x-axis) in addition to AAV9 and AAV^{mut1}. Log 2Fold DNA levels of AAV9 and AAV^{mut1} are identified specifically in FIG. 59.

FIG. 60 is a box plot showing the distribution of liver RNA log 2FC enrichment score from capsids in all sequence groups defined by P₃, P₅, P₆, P₈ positions (see also positions P₃, P₅, P₆, P₈ in VR I).

FIG. 61 is a plot showing liver enrichment for rAAV from AAV^{-Lib1} in mice (y-axis) versus liver enrichment for rAAV from AAV^{-Lib1} in NHP (x-axis) Correlation is indicated by 0.72 R\hat{ }2-value. Capsids plotted on FIG. 61 include capsids having a peptide segment with a P3TPSGP6G motif.

FIGS. 62A-62E shows non-limiting examples of combination of targeting peptides, comprising: variable triplets, a constant RGD, a variable quad, and, optionally, peptide segments in the AAV-Lib3 library in Example 27. FIG. 62A shows possible amino acid residues in the variable triplet corresponding X₁, X₂, and X₃ where the color indicates the type of amino acid. FIG. 62B shows the constant RGD quad that is in each targeting peptide. FIG. 62C shows the amino acid residue category (genus) and specific quads. FIG. 62D shows non-limiting examples of peptide segments present in VR I. FIG. 62E shows amino acids at positions X₇, X₈, X₉, and X₁₀, where the color indicates the type of amino acid residue.

FIGS. 63A-63B show an enrichment plots (Avg_logFC versus Inverse CV (Coefficient of Variation)) for capsids in the AAV^{-Lib2} library. FIG. 63A is an enrichment plot showing capsid performance in muscle (i.e., all muscle tissue) for all 50,500 capsids in the AAV^{-Lib2} library. FIG. 63B shows enrichment plots for capsids from the AAV^{-Lib2} library having a quad selected from: FNNL, FNNT, and RGQI.

FIG. 64 shows an enrichment plot of the amount of vector RNA in muscle tissue for the sum of all the capsids in the AAV^{-Lib2} library having the indicated quad sequences.

FIG. 65 shows a heatmap of tissue enrichment scores in muscle tissues for each of the capsids in the AAV^{-Lib2} library. AAV^{-Lib2} library includes about 50,500 capsids having one of the 20 quad and one of XX triplet sequences. Data for each capsid appears as a cell in the heatmap where the row indicates the quad and the column indicates the triplet. Data is grouped using hierarchical clustering. Clustering identified Group I, Group II, and Group III.

FIG. 66A shows enrichment scores averaged across all muscle tissue for each of the capsids in the AAV^{-Lib2} library having a quad selected from: RGQI, RSVV, and RGVV (Group 1). The top 100 capsids are identified by the larger of the two dots on the plot.

FIG. 66B shows a motif plot of the targeting peptides for the top 100 capsids in the AAV^{-Lib2} library having a quad selected from: RGQI. RSVV, and RGVV (Group 1). Data used to generate the motif plot is averaged across all muscle tissue.

FIG. 66C shows a plot of unique variant count for the indicated triplets for the top 100 capsids in the AAV^{-Lib2} library having a quad selected from: RGQI, RSVV, and RGVV (Group 1).

FIG. 67A shows enrichment scores averaged across all muscle tissue for each of the capsids in the AAV^{-Lib2} library having a quad selected from: HGVL, YSSV, YSTM, and YTSV (Group 2). The top 100 capsids are identified by the larger of the two dots on the plot.

FIG. 67B shows a motif plot of the targeting peptides for the top 100 capsids in the AAV^{-Lib2} library having a quad selected from: HGVL, YSSV, YSTM, and YTSV (Group 2). Data used to generate the motif plot is averaged across all muscle tissue.

FIG. 67C shows a plot of unique variant count for the indicated triplets for the top 100 capsids in the AAV^{-Lib2} library having a quad selected from: HGVL, YSSV, YSTM, and YTSV (Group 2).

FIG. 68A shows enrichment scores averaged across all muscle tissue for each of the capsids in the AAV^{-Lib2} library having a quad selected from: FNNT, FNNL, FONT, and YNSL (Group 3). The top 100 capsids are identified by the larger of the two dots on the plot.

FIG. 68B shows a motif plot of the targeting peptides for the top 100 capsids in the AAV^{-Lib2} library having a quad selected from: FNNT, FNNL, FONT, and YNSL (Group 3). Data used to generate the motif plot is averaged across all muscle tissue.

FIG. 68C shows a plot of unique variant count for the indicated triplets for the top 100 capsids in the AAV^{-Lib2} library having a quad selected from: FNNT, FNNL, FONT, and YNSL (Group 3).

FIG. 69 shows an enrichment plot of the amount of vector RNA in liver tissue for the sum of all the capsids in the AAV^{-LIB2} library having the indicated quad sequences.

FIG. 70A shows enrichment scores for liver tissue for each of the capsids in the AAV^{-Lib2} library having a quad selected from: RGQI, RSVV, RGVV, RQGI, and RSQT. The top 100 capsids are identified by the larger of the two dots on the plot.

FIG. 70B shows a motif plot of the targeting peptides for the top 100 capsids in the AAV^{-Lib2} library (from FIG. 70A) having a quad selected from: RGQI, RSVV, RGVV, RQGI, and RSQT. Data used to generate the motif plot is averaged across the liver tissue.

FIG. 71A shows an enrichment score plots (inverse VC versus AverageMN_FC) for biceps femoris, quadriceps, diaphragm, heart-atria, heart-ventricle, and liver for each of the capsids in the AAV^{-Lib2} library. The top 10 performing capsids are identified by the darker shaded circles.

FIG. 71B shows a table of enrichment scores for the top 10 capsids, where the top 10 capsids include the indicated targeting peptide.

FIG. 71C shows a table of the amino acid residues (at each position for the targeting peptides described in FIG. 71B and shown also in FIG. 71A.

FIG. 72A shows an enrichment score plot (Log FC mean versus inverse CV (count of variants)) for capsids having the indicated quads. Targeting peptides with specific triplets are identified.

FIG. 72B shows a table of the amino acid residues at each position or the targeting peptides described in FIG. 72A.

FIG. 73A shows enrichment score plot (Log FC mean versus inverse CV (count of variants)) for capsids having the indicated quads. Targeting peptides with specific triplets are identified.

FIG. 73B shows a table of the amino acid residues (and properties for the respective amino acid residues (e.g., polar)) for the targeting peptides described in FIG. 73A

FIG. 74A shows enrichment scores in skeletal muscle versus heart for each of the capsids in the AAV^{-Lib2} library. The 10 capsids are highlighted.

FIG. 74B shows enrichment scores in skeletal muscle versus heart for each of the capsids in the AAV^{-Lib2} library. The 10 capsids are highlighted.

FIG. 75A shows enrichment scores in skeletal muscle versus enrichment scores heart for each of the capsids in the AAV^{-Lib2} library.

FIG. 75B shows enrichment scores in skeletal muscle versus enrichment scores in heart for each of the capsids in the AAV^{-Lib2} library having the quad “LIGR”.

FIG. 75C shows enrichment scores in skeletal muscle versus enrichment scores in heart for each of the capsids in the AAV^{-Lib2} library having the quad (“QSTL”).

FIG. 75D shows enrichment scores in skeletal muscle versus enrichment scores in heart for each of the capsids in the AAV^{-Lib2} library having the quad (“RGVV”).

FIG. 75E shows enrichment scores for heart over enrichment scores in skeletal muscle for each of the capsids in the AAV^{-Lib2} library having the indicated quads.

### 6. DETAILED DESCRIPTION

### 6.1. Definitions

“AAV” is adeno-associated virus and may be used to refer to the virus itself or derivatives thereof. The term covers all subtypes, serotypes and pseudotypes, and both naturally occurring and recombinant forms, except where required otherwise.

The term “AAV capsid protein” or simply “capsid protein” refers to a VP1, VP2, or VP3 capsid protein. In some embodiments, the AAV capsid protein is a wild type or modified capsid protein of AAV9; AAV2; AAV1; AAV6; AAV3; AAV LK03; AAV7; AAV8; AAV hu.37; AAV rh.10; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu.9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74, hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80159; Anc80L60; Anc80162; Anc80L65; Anc80L33; Anc80L36; Anc80L44; Anc80L1; Anc110; and Anc80DI.

The term “modified AAV capsid protein” or simply “modified capsid protein” refers to a capsid protein that is modified as compared to such naturally occurring or synthetic/artificial capsid protein, which is referred to as the “reference AAV capsid protein” or “reference capsid protein.” The reference AAV capsid protein as used herein may be a VP1, VP2, or VP3 capsid protein of a naturally occurring AAV variant or a non-naturally occurring VP1, VP2, or VP3 capsid protein that is known in the art.

The term “targeting peptide” as used herein refers to a 10 amino acid sequence (X₁X₂X₃RGDX₇X₈X₉X₁₀) within the variable region VIII (VRVIII) of a modified AAV capsid protein introduced by one or more modifications described herein. AAVs comprising a modified capsid protein with a targeting peptide can have localization and distribution in a target cell, tissue or organ different from the AAV with a capsid protein without the target peptide.

The term “peptide segment” as used herein refers to a part of variable region I (VR I) of an AAV capsid protein comprising 10, 11, or 12 amino acids. In preferred embodiments, the peptide segment is positioned between amino acid 250 and 280 of the AAV capsid protein. A modified AAV capsid protein provided herein includes a peptide segment having a sequence different from the corresponding sequence of a reference AAV capsid protein by having one or more modifications. The peptide segment can be referred to as P₁P₂P₃P₄P₅P₆P₇P₈P₉P₁₀P₁₁P₁₂ within VR I where “Pn” refers to a position in the peptide segment.

The term “amino acid position” within an AAV capsid protein as here herein refers to a position of an amino acid residue in an AAV VP1 protein sequence, counted from the first amino acid in the N terminal. As used herein, the term “amino acid” comprises naturally occurring L- and D-amino acids and artificial, i.e. non-naturally occurring, α-amino acids. Preferably, the amino acid is a naturally occurring amino acid. In preferred embodiments, the amino acid is a naturally occurring L-α-amino acid.

For the avoidance of doubt, as used herein, the indication that an insertion site is at amino acid position X means that the targeting peptide is inserted between amino acids X and X+1. i.e., the targeting peptide is inserted after the indicated amino acid.

The term “liver off” is used herein to describe an AAV having a lower tropism to liver or less biodistribution in liver when administered to a mammalian subject compared to other AAV variants. The term “liver off” is also used to describe a modification in the AAV capsid protein that reduces the tropism to liver or biodistribution in liver when administered to a mammalian subject. The term “liver off” is also used to describe a species of an AAV capsid with a variable region 1 (VRI) that reduces the tropism to liver or biodistribution in liver when administered to a mammalian subject.

The term “liver on” is used herein to describe an AAV having a higher tropism to liver or more biodistribution in liver when administered to a mammalian subject compared to other AAV variants. The term “liver on” is also used to describe a modification in the AAV capsid protein that increases the tropism to liver or biodistribution in liver when administered to a mammalian subject.

The term “CAG” when used in relation to a promoter or ERE refers to a promoter or ERE with chicken beta actin promoter and CMV enhancer sequences.

The term “constitutive” promoter or ERE as used herein refers to a nucleotide sequence which, when operably linked with a polynucleotide which encodes or specifies a gene product, causes the gene product to be produced in a cell under most or all physiological conditions of the cell.

The term “expression regulatory element” or “ERE” as used herein in the context of the rAAV of the disclosure refers to a nucleic acid sequence which is required for expression of the MTM1 coding sequence operably linked to the ERE. In some instances, an ERE sequence may be the core promoter sequence and in other instances, this sequence may also include an enhancer sequence and other regulatory elements which are required for expression of the gene product, for example exon sequences.

The term “functional fragment” in the context of the myotubularin or MTM1 refers to a biologically functional fragment of myotubularin or MTM1. As would be understood in the art, a biologically functional fragment is a portion or portions of a full length sequence that retain a biological function of the full length sequence. An exemplary functional fragment corresponds to amino acids 29-486 of SEQ ID NO: 165 (and is disclosed herein as SEQ ID NO:164). Biological functions of MTM1 include the ability cleave or hydrolyze an endogenous phosphoinositide substrate known in the art, or an artificial phosphoinositide substrate for in vitro assays (i.e., a phosphoinositide phosphatase activity), to recruit and/or associate with other proteins such as, for example, the GTPase Rab5, the PI 3-kinase Vps34 or Vps15 (i.e., proper localization), or treat myotubular myopathy.

The term “functional variant” in the context of the myotubularin or MTM1 refers to various splicing isoforms, variants, fusion proteins, and modified forms of the wildtype MTM1 polypeptide or a functional fragment thereof. Such isoforms, bioactive fragments or variants, fusion proteins, and modified forms of the MTM1 polypeptides retain at least one biological function of the full length MTM1 protein (e.g., a protein of SEQ ID NO: 165).

The term “inducible” promoter or ERE as used herein refers to a nucleotide sequence which, when operably linked with a polynucleotide which encodes or specifics a gene product, causes the gene product to be produced in a cell substantially only when an inducer which corresponds to the promoter is present in the cell.

As used herein, the term “internalizing moiety” refers to a moiety capable of interacting with a target tissue or a cell type to effect delivery of the attached molecule into the cell (i.e., penetrate desired cell; transport across a cellular membrane). In certain embodiments, an MTM1 polypeptide encoded by the rAAV of the disclosure can be a fusion protein comprising an internalizing moiety. In some embodiments, the internalizing moiety selectively, although not necessarily exclusively, targets and penetrates muscle cells. In certain embodiments, the internalizing moiety has limited cross-reactivity, and thus preferentially targets a particular cell or tissue type. In certain embodiments, suitable internalizing moieties include, for example, antibodies, monoclonal antibodies, or derivatives or analogs thereof. Other internalizing moieties include for example, homing peptides, receptors, and ligands. In certain embodiments, the internalizing moiety mediates transit across cellular membranes via an ENT2 transporter. Exemplary internalizing moieties are disclosed in U.S. Pat. No. 9,447,394 B2, the contents of which are incorporated by reference herein.

The term “inverted terminal repeat” (or “ITR”) refers to a polynucleotide sequence found at the ends of AAV genomes that form a hairpin, which contributes to the genome's ability to self-prime (allowing for primase-independent synthesis of the complementary second DNA strand) and provides for encapsidation of the genome into an AAV particle. An ITR can be a wild-type ITR or a variant thereof.

The terms “liver-toggle mutant”, “liver-toggle mutant of a reference AAV capsid protein” and the like, as used herein, refers to a capsid protein comprising a sequence different from a reference AAV capsid protein by having one or more mutations (e.g., amino acid substitutions) that alter tropism, specificity or distribution in a liver as compared to the reference AAV capsid protein when administered to a mammalian subject (such a sequence difference referred to herein as a “liver toggle mutation”). The mammalian subject can be a human, non-human primate (NHP), mice, rats, birds, rabbits, guinea pigs, hamsters, farm animals (including pigs and sheep), dogs, or cats. Exemplary liver toggle mutations are disclosed in WO2019/217911 and WO2021/050614, incorporated by reference in their entireties herein. In some embodiments, the liver toggle mutations comprise (i) an alanine (A) or guanine (G) amino acid residue at an amino acid position corresponding to position 266 in Anc80 VP1 and/or b) a lysine (K) or arginine (R) amino acid residue at an amino acid position corresponding to position 168 in Anc80 VP1. In other embodiments, a liver-toggle mutant of a reference AAV capsid protein is a capsid protein comprising a sequence different from the reference AAV capsid protein by having an alanine (A) amino acid residue at an amino acid position corresponding to position 267 in AAV9 VP1 protein and a threonine (T) amino acid residue at an amino acid position corresponding to position 269 in AAV9 VP1. In yet further embodiments, the liver toggle mutations comprise a sequence different from the reference AAV capsid protein by having any combination of (i) an arginine (R) instead of serine (S) at position 446; (ii) an alanine (A) instead of an arginine (R) at position 471; and (iii) a threonine (T) or alanine (A) instead of a valine (V) at position 708, in each case numbered according to an AAV2 reference capsid protein (SEQ ID NO: 1 of WO2021/050614, which is incorporated by reference herein).

The term “modification” when in conjunction with an amino acid residue, amino acid residues, or a modified sequence, refers to insertion(s), deletion(s), and/or substitution(s).

The term “MTM1 coding sequence” is used herein to refer to a specific sequence of nucleotides in a polynucleotide, such as an rAAV genome or mRNA produced thereby, that encodes an MTM1 polypeptide.

The term “MTM1 polypeptide” refers to a polypeptide comprising an amino acid sequence having at least 90%, at least 95%, at least 98%, at least 99% or 100% identity to human MTM1 (SEQ ID NO: 165) or a functional fragment (e.g., SEQ ID NO: 164) or functional variant thereof.

The terms “operably linked” and “operatively linked” refer to the functional relationship of the nucleic acid sequences with regulatory sequences of nucleotides, such as promoters, enhancers, transcriptional and translational stop sites, and other signal sequences and indicates that two or more DNA segments are joined together such that they function in concert for their intended purposes. For example, operative linkage of nucleic acid sequences, typically DNA, to a regulatory sequence or promoter region refers to the physical and functional relationship between the DNA and the regulatory sequence or promoter such that the transcription of such DNA is initiated from the regulatory sequence or promoter, by an RNA polymerase that specifically recognizes, binds and transcribes the DNA.

The term “parenteral” administration of a composition includes, e.g., subcutaneous (s.c.), intravenous (i.v.), intramuscular (i.m.), or intrasternal injection, or infusion techniques.

The terms “peptide”, “polypeptide” and “protein” are used interchangeably herein to refer to a polymer of amino acid residues.

The term “pharmaceutically acceptable carrier” includes any of the standard pharmaceutical carriers, excipients, stabilizers and adjuvants. For examples of carriers, excipients, stabilizers and adjuvants, see Remington: The Science and Practice of Pharmacy, 22nd Revised Ed., Pharmaceutical Press, 2012.

The abbreviation “rAAV” refers to a recombinant adeno-associated viral particle composed of at least one AAV capsid protein and an encapsidated polynucleotide, sometimes referred to herein as a “genome”. rAAV can include a genome that comprises a heterologous polynucleotide (i.e., a polynucleotide other than a wild-type AAV genome), such as a heterologous polynucleotide encoding a gene delivered to a mammalian cell such as the MTM1 gene. The heterologous nucleotide is sometimes referred to as a transgene.

The term “self-complementary” rAAV vector or genome as used herein means a fully or partially self-complementary rAAV vector or genome, respectively. A “fully self-complementary” rAAV vector refers to a vector containing a genome generated by the absence of a terminal resolution site (TR) from one of the ITRs of the rAAV. The absence of a TR prevents the initiation of replication at the vector terminus where the TR is not present. In general, fully self-complementary rAAV vectors generate single-stranded, inverted repeat genomes, with a wild-type (wt) AAV TR at each end and a mutated TR (mTR) in the middle. Thus, a fully self-complementary rAAV genome is typically a single stranded polynucleotide having, in the 5′ to 3′ direction, a first ITR sequence, a heterologous sequence (e.g., MTM1 coding sequence and/or ERE), a second ITR sequence, a second heterologous sequence that is complementary to the first heterologous sequence, and a third ITR sequence. A “partially self-complementary” rAAV genome refers to a single stranded polynucleotide having, in the 5′ to 3′ direction or the 3′ to 5′ direction, a first ITR sequence, a heterologous sequence (e.g., MTM1 coding sequence and/or ERE), a second ITR sequence, and a self-complementary region that is complementary to a portion of the heterologous sequence and has a length that is less than the entire length the heterologous sequence.

The term “tissue-specific” promoter or ERE as used herein refers to a nucleotide sequence which, when operably linked with a polynucleotide encodes or specified by a gene, causes the gene product to be produced in a cell substantially only if the cell is a cell of the tissue type corresponding to the promoter.

The terms “treatment”, “treating”, and the like are used herein to generally mean obtaining a desired pharmacologic and/or physiologic effect. The effect may be prophylactic in terms of completely or partially preventing a disease, condition, or symptoms thereof, and/or may be therapeutic in terms of a partial or complete cure for a disease or condition and/or adverse effect attributable to the disease or condition. “Treatment” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms).

The terms “vector”, “AAV vector” and “rAAV vector” refer to an rAAV that comprises a heterologous polynucleotide, e.g., a transgene.

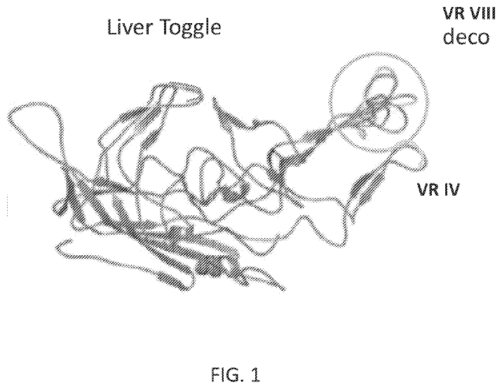

The terms “variable region” or “VR” as used herein refer to one or more of nine sequence variable regions (e.g., VRI to VRIX) in an AAV capsid protein previously defined by comparison and alignment of various AAV capsid proteins. See e.g., Govindasamy et al., Structurally mapping the diverse phenotype of adeno-associated virus serotype 4, J. Virol (2006); Meyer et al. Structure of the gene therapy vector, adeno-associated virus with its cell receptor, AAVR, eLife (2019). The VRs are known to contain amino acids that contribute to slight differences in surface topologies and distinct functional phenotypes, such as in receptor binding, transduction efficiency, and antigenic re-activity. The relative positions of the VR I, VR IV and VIIII are illustrated in FIG. 1, but the specific positions of the variable regions within a capsid protein can vary depending on the capsid protein and/or the sequence alignment method.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### 6.2. Modified AAV Capsid Proteins

One aspect of the present disclosure provides a modified adeno-associated virus (AAV) capsid protein comprising (i) a targeting peptide at a site within variable region VIII (VR VIII); or (ii) a peptide segment within variable region I (VR I), wherein the targeting peptide has a sequence of X₁X₂X₃RGDX₇X₈X₉X₁₀, wherein X₁, X₂, X₃, X₇, X₈, X₉ and X₁₀ are independently selected from any amino acid residue, wherein the peptide segment has an amino acid sequence of P₁P₂P₃P₄P₅P₆P₇P₈NDNP₁₂ and P₁, P₂, P₃, P₄, P₅, P₆, P₇, P₈, and P₁₂ are independently selected from any amino acid residue. In some embodiments, the modified AAV capsid protein comprises both (i) the targeting peptide and (ii) the peptide segment. In some embodiments, the AAV capsid protein comprises the targeting peptide but not the peptide segment. In some embodiments, the AAV capsid protein comprises the peptide segment but not the targeting peptide.

One aspect of the present disclosure provides a modified adeno-associated virus (AAV) capsid protein comprising: a sequence of a reference AAV capsid protein comprising: (i) a targeting peptide at a site within variable region VIII (VR VIII) of the reference AAV capsid protein; and (ii) one or more modifications to comprise a peptide segment within variable region I (VR I) of the reference AAV capsid protein, wherein the targeting peptide has a sequence of X₁X₂X₃RGDX₇X₈X₉X₁₀, wherein X₁, X₂, X₃, X₇, X₈. X₉ and X₁₀ are independently selected from any amino acid residue, wherein the peptide segment has an amino acid sequence of P₁P₂P₃P₄P₅P₆P₇P₈NDNP₁₂ and P₁, P₂, P₃, P₄, P₅, P₆, P₇, P₈, and P₁₂ are independently selected from any amino acid residue.

In some embodiments, the modified AAV capsid protein does not comprise RGDLLLS (SEQ ID NO: 1).

In some embodiments, the modified AAV capsid protein has a peptide segment that does not comprise an alanine (A) at P₆ and a threonine (T) at P₈

In some embodiments, the modified AAV capsid does not comprise RGDLLLS (SEQ ID NO: 1), and the peptide segment does not comprise an alanine (A) at P₆ and a threonine (T) at P₈.

In some embodiments, the modified AAV capsid protein has one or more amino acid insertions, deletions, substitutions, or combinations thereof as compared to a reference AAV capsid.

In some embodiments, the reference AAV capsid protein further comprises one or more modifications comprising an amino acid insertion, deletion, substitution, or a combination thereof to introduce the targeting peptide within VR VIII of the reference AAV capsid protein.

In some embodiments, the capsid protein further comprises one or more modifications outside of the VR I and VR VIII of the reference AAV capsid protein.

In some embodiments, the reference AAV capsid protein includes one or more modifications comprising an amino acid insertion, deletion, substitution, or a combination thereof to introduce the targeting peptide within VR VIII (e.g., wherein VR VIII corresponds to amino acids between positions 565 and 595 of the reference AAV capsid protein).

In some embodiments, the reference AAV capsid protein includes one or more modifications outside of VR I (e.g., VR I corresponds to amino acids between position 259 to position 275 of the reference AAV capsid protein) of the reference capsid protein. In some embodiments, the reference AAV capsid protein includes one or more modifications outside of VR VIII (e.g., VR VIII corresponds to amino acids between positions 565 and 595 of the reference AAV capsid protein) of the reference AAV capsid protein. In some embodiments, the reference AAV capsid protein includes one or more modifications outside of VR I (e.g., VR I corresponds to amino acids between position 259 to position 275 of the reference AAV capsid protein) and VR VIII (e.g., VR VIII corresponds to amino acids between positions 565 and 595 of the reference AAV capsid protein) of the reference AAV capsid protein.

In some embodiments, the one or more modifications is an amino acid insertion, deletion, substitution, or a combination thereof to introduce the targeting peptide within VR VIII of the reference AAV capsid protein. In some embodiments, the one or more modifications is an amino acid insertion, substitution, or a combination in a region outside of the VR VIII region of the reference AAV capsid protein. In some embodiments, the one or more modifications outside of the VR VIII is a modification in the VR I region of the reference AAV capsid protein. In some embodiments, the one or more modifications results in a liver-off phenotype. In some embodiments, the one or more modifications outside of the VR VIII is a liver toggle mutation.

### 6.2.1. Targeting Peptide

In some embodiments, the targeting peptide has a sequence X₁X₂X₃RGDX₇X₈X₉X₁₀, wherein X₁, X₂, X₃, X₇, X₈, X₉ and X₁₀ are independently selected from any amino acid residue. In some embodiments, X₇, X₈, X₉ and X₁₀ are independently selected from A, D, E, F, G, H, I, K, L, N, Q, R, S, T, V, and Y. In some embodiments, X₇, X₈, X₉ and X₁₀ are independently selected from A, D, E, F, G, H, I, K, L, N, Q, R, S, T, and V.

In some embodiments, the modified AAV capsid protein has at least 90% sequence identity to the sequence of the reference AAV capsid protein. In some embodiments, the modified AAV capsid protein has at least 95% sequence identity to the sequence of the reference AAV capsid protein. In some embodiments, the modified AAV capsid protein has at least 96% sequence identity to the sequence of the reference AAV capsid protein. In some embodiments, the modified AAV capsid protein has at least 97% sequence identity to the sequence of the reference AAV capsid protein. In some embodiments, the modified AAV capsid protein has at least 98% sequence identity to the sequence of the reference AAV capsid protein. In some embodiments, the modified AAV capsid protein has at least 99% sequence identity to the sequence of the reference AAV capsid protein.

In some embodiments, X₁, X₂, and X₃ are independently selected from any amino acid residue.

In yet other embodiments, X₁, X₂, and X₃ have not been modified from the amino acids at corresponding positions of the reference AAV capsid protein. In this case, X₁, X₂, and X₃ are identical to the amino acids at corresponding positions of the reference AAV capsid protein.

In yet other embodiments, X₁, and X₂ have not been modified from the amino acids at corresponding positions of the reference AAV capsid protein. In yet other embodiments, X₁ has not been modified from the amino acids at corresponding position of the reference AAV capsid protein. In yet other embodiments, X₂ has not been modified from the amino acids at corresponding position of the reference AAV capsid protein. In some embodiments, X₁, X₂, and/or X₃ are the natural amino acid residues of the reference AAV capsid protein.

X₁, X₂, and X₃ can be introduced by amino acid substitutions, insertions, mutations, and/or deletions of the amino acids at the corresponding sites in the reference AAV capsid. For example, in some embodiments, any one of the corresponding sites X₁, X₂, and X₃ in the reference AAV capsid can be deleted or substituted. In some embodiments, the corresponding site X₁ in the reference AAV capsid is deleted or substituted. In some embodiments, the corresponding site X₂ in the reference AAV capsid is deleted or substituted. In some embodiments, the corresponding site X₃ in the reference AAV capsid is deleted or substituted. In some embodiments, the corresponding sites X₁, and X₂ in the reference AAV capsid are deleted or substituted. In some embodiments, the corresponding sites X₂ and X₃ in the reference AAV capsid are deleted or substituted. In some embodiments, the corresponding sites X₁ and X₃ in the reference AAV capsid are deleted or substituted. In some embodiments, the corresponding sites X₁, X₂, and X₃ in the reference AAV capsid are deleted or substituted.

In some embodiments, X₁ is selected from S, E, A, D, N, Q, or T. In some embodiments, X₁ is D or E. In some embodiments, X₁ is S, A or T. In some embodiments, X₁ is S, A or E. In some embodiments, X₁ is selected from S or E. In some embodiments, X₁ is S. In some embodiments, X₁ is E.

In some embodiments, X> is selected from N, A, D, E, F, G, H, I, K, L, M, N, P, Q, R, S, T, V, W, or Y. In some embodiments, X₂ is selected from Nor A. In some embodiments, X₂ is selected from K, E, D, A, S, F, N, V or L. In some embodiments, Xe is selected from K, E or D. In some embodiments, X₂ is selected from N, A or Y. In some embodiments, X₂ is selected from N, Y or S.

In some embodiments, X₃ is selected from R, Q, A, D, E, F, G, H, L, K, L, M, N, P, S, T, V, W, or Y. In some embodiments, X₂ is N. In some embodiments, X₃ is A. In some embodiments. X₃ is selected from R or Q. In some embodiments, X₃ is R. In some embodiments, X₃ is Q. In some embodiments, X₂ is N; and X₃ is R. In some embodiments, X₁ is E; X₂ is N; and X₃ is R. In some embodiments, X₁ is S; X₂ is N; and X₃ is R. In some embodiments, X₃ is selected from Y, V or F. In some embodiments, X₃ is selected from N, A, Y or S. In some embodiments, X₃ is selected from I, Q, R, V, T or M.

In some embodiments, X₂X₃ is NR. In some embodiments, X₁X₂X₃ is ENR.

In some embodiments, X₁X₂X₃ is SNR.

In some embodiments, X₁X₂X₃ is selected from the groups consisting of: DII, DWM, EEI, DML, DWI, SLE, EIN, NHE, DFI, EEL, TEQ, TDA, EDT, NEV, TDW, QFE, EDY, DTT, EPL, SEN, SEQ, TAE, EVN, ELN, DVQ, ETI, EVI, ESV, ETW, SEW, DNW, EVF, EAW, EPF, EIY, EIF, EPY, DVI, DMM, DQI, DHL, DTL, DVL, NDL, DLL, DMQ, NEF, DFL, DIM, TEW, DYI, SDY, DYY, DHF, DKE, DTW, DTI, ELY, TEY, TEI, DAI, DQY, DMY, EWG, DMV, DMI, EPH, QEG, DIN, NEI, EYY, DIV, SEG, DVG, DYQ, EGF, NDI, EGY, DVF, DVH, DGF, DIY, DSF, DGW, EHY, DRE, TEH, DTS, NEN, NEM, NEH, TEN, DSN, DVT, DQS, DKD, DTH, DVV, DQK, NET, DKP, TEV, NDS, QET, EVL, SET, SDT, AEQ, QEF, SEY, SEF, SPF, EGQ, ETH, TDQ, QEA, QDQ, AEN, ESS, NDT, EFM, EFI, EHM, DFM, QDT, SME, DYT, EHV, ENV, EAV, EAI, ESI, DAT, ENQ, EAM, ADN, EFQ, SDS, TDH, SDH, DAS, TWE, SSF, DRD, EFL, TDF, QDA, EMH, SGE, AEW, DAH, TET, TDM, TNE, SAE, NSE, SFE, QDI, DSA, ADV, SEI, AEI, QDV, ADT, DNM, DNQ, ADL, TDL, SDL, SDM, TDV, DNI, DHY, DTY, DAA, DSY, QLY, DVM, DAY, DMT, DQT, DAQ, DTV, DSH, QDF, DST, DNL, DSI, DFV, DNY, DAF, DKI, DKF, DTM, DSL, NDV, TDI, DSV, DAV, DKV, DAM, DNV, DKM, DKL, DKW, DSM, ENI, SDI, DKT, QGE, NNE, QYW, AGM, ESH, QDH, QEH, DYH, ENS, DKQ, TGE, DSS, EST, DSQ, DNS, EFV, SWE, DKN, DKS, SEH, EAL, EAS, ADQ, SDQ, ADI, SDV, EAH, ASE, EAF, EYV, DKA, SNE, AGE, EGV, EQF, ETF, EVY, EQY, QEY, QEN, SAD, AEF, EMF, SDF, ADF, ADY, AVF, TLY, SIY, SLY, ATY, DYV, AEY, ENF, EMY, QDY, ETY, EAY, TVY, SVY, QVF, QVY, TMY, DKY, ALY, AVY, TMW, EQW, SPW, SIW, DTQ, TFF, AMF, EYT, DRV, SPY, SIF, SLF, ENY, EWY, EYI, EFY, TLF, TVF, TYY, TFY, SVF, SYW, TAW, SAW, TIY, NIM, NFM, NII, NIV, SII, SIV, SEM, TTI, NSY, SSY, SNY, SEV, AEH, ALT, SGQ, QTY, SYY, AMY, QMY, SGF, SGY, ALF, TTF, SFN, QAF, ASF, SAF, TAF, TAY, TGL, SAY, QAY, ADH, ASI, SSV, SSI, TGV, SGV, DPH, TEG, DLY, ESE, TEF, NPE, DHA, DLI, DWG, EWM, EET, EIL, TEL, DYL, EMW, EVW, EPV, EDV, SLD, EGH, QRY, TDG, SQE, ELH, EYW, EMM, EFF, QEI, EQG, DVS, ESP, EIQ, EPM, EPQ, SPE, EPN, NQD, ELQ, TDT, ELT, EPI, ETM, ETG, EHI, EMI, EIT, ETV, EVV, EVM, TPY, QEW, EIW, EPT, EIH, DFY, DQL, NDA, DIW, TDY, ELF, DVY, ESG, EPG, EIG, ELG, EVG, DHI, EIS, DYM, DFT, DFH, EQS, TPF, EEF, DWN, SDN, EYL, AET, DMN, QSP, DNA, DHM, DHT, EQL ESN, EYF, EHF, NAD, AEG, AES, TEM, SEL, DYS, EVH, EAN, EGL, TFE, ETL, TYE, SHE, ELM, TYD, SYE, ESA, EFT, EAT, AMD, AND, DMS, QMD, SLQ, EYS, QDN, EYQ, EKD, QYD, QFD, DER, AFD, EAA, EFS, DHV, QSE, TSD, SFD, DGY, TWD, QWD, EHL, QGD, QSD, AWD, AGD, QIY, EMQ, EAG, ERD, DDR, DAL, QDL, DNT, EGN, ENH, NGE, DYN, DNH, DNF, ESQ, ESM, ESL, SSD, ENL, EMV, QDS, TSE, SWD, ASD, ENT, SSE, EGM, EGS, EGT, EGI, AIF, NIN, NIQ, NYW, EPW, EGG, TIW, QIW, TIW, EKW, NLY, SVM, TPT, AAW, AIH, TIH, QGT, QFF, QYF, QFY, EHH, QLH, TYF, STY, DRL, EYM, SFF, QLF, QIF, APF, TYW, QII, QPF, SPI, TIL, SVL, NVI, SIM, SLL, QEL, AEM, AEL, QEV, QDM, NDM, SYL, ASL, TPL, ANI, NMI, TIM, AEV, TMI, SWI, SFI, SNW, TNF, NAF, ASP, QSQ, APY, SFM, TAN, QAW, SGS, SGH, TGF, QSN, ERY, QMN, TGQ, NGM, NGV, AMN, ALN, QFV, AAF, TFV, TSY, EDK, ASN, SAV, QSV, QGV, QST, ASM, TAI, QSM, QSL, QSI, SSL, AGV, TGT, AGI, QGM, TGM, SGM, SGI, THD, QVE, ALE, QLE, EWP, AFP, AEE, ADE, NIE, TYP, QIE, TFP, SYP, ELW, DVW, NEW, EPA, EVA, TLE, AWE, NME, QME, ATE, AYE, DQM, TPD, DFG, QPE, EDI, EWL, ELL, QPD, EWF, ALD, SVD, QVD, ADA, APD, AHE, EQL, DTA, AFE, DAW, QAE, TME, STE, THE, QID, AAE, EIA, EML, SDA, SIE, TQE, TVD, NWE, NOE, EYG, QYP, EIV, EII, QFP, TID, EQN, EVS, TYG, EAQ, ELV, QLD, TLD, QQD, TIN, TLN, SPN, TPN, NFD, NEY, DSW, NMD, DGA, DYA, DIH, DNP, DQW, DQV, EFA, EPS, DEN, DWV, ELI, DTF, EQT, DHW, ESF, DEK, DMH, EWS, EHA, TTD, EWI, QLQ, SQD, QHD, NSD, EMA, TND, ATD, DGS, TAD, TMD, QES, AHD, QAD, DGH, DGL, NTE, ENG, SMD, NDQ, EMS, ENA, EMT, EYN, DDK, EWA, EWQ, EFH, QWE, DGT, DGM, EWV, EWH, EWT, QND, DGV, ESY, AAD, SND, ANE, TIG, QIG, AVD, SFP, AWP, QDW, SLW, TPA, AII, TIV, SWP, AFY, APN, AVN, TVN, TIS, TIQ, TLQ, AIQ, QVN, NFY, QFG, EMN, NVF, SYF, SFW, TPQ, AIL, TWI, QWN, TWN, QWQ, AQF, ASA, TWV, EWN, TMF, NGI, ATN, SAS, TMQ, TMN, AYN, TQF, SMF, QMQ, SMQ, QAN, AAY, SFQ, TYQ, TFQ, TWQ, AVI, and SGL.

In some embodiments, X₁X₂X₃ is selected from the group consisting of: DAV, DKW, EAY, AEY, DFV, DKF, DKI, DKL, DNV, DNY, DSL, DSV, EFI, SEF, SEY, SLY, ADF, ADY, ALY, AVF, DAF, DAL, DAM, DAT, DHV, DIV, DKA, DKM, DKT, DKV, DKY, DMI, DNF, DNI, DQT, DSI, DVY, DYN, DYV, EAT, EAW, EFV, EGL, EIY, EMF, EMY, ENF, EPF, EPY, EQY, ESY, ETF, EWI, EWT, EYI, EYV, NEM, QDF, QDY, QEY, QLY, QND, QVF, QVY, SDL, SDV, SEH, SII, SIY, SSL, SVY, SWD, SWE, TDF, TDV, TDY, TEF, TEY, TIY, TLY, and TWQ.

In some embodiments, X₁X₂X₃ is selected from the group consisting of: APW, TEL, TDA, QPY, SPN, EHY, DWK, DLK, DFK, DVK, NSI, DIR, SPF, SEL, DRT, DRF, ADL, TDL, SDL, DNY, DKI, NDV, DKM, DNH, DNF, DSS, EST, EWT, DKN, DKS, SEH, ESQ, ESL, QND, EAH, AIF, AVF, QVF, TMY, ALY, NNG, NIF, NTF, NFF, AWF, NPY, SWF, AII, AYF, AQW, NFY, AGP, QQF, TKE, TNG, NSF, NAW, QAG, ERG, NKD, QSG, QNG, EAK, QWF, SWY, TFF, TYF, NYY, QFG, NWA, AMF, STY, TNW, ANW, AWM, TSF, DRL, DRV, SPY, NVF, SIF, QLF, SLF, QIF, SYF, APF, AGF, SVF, SAW, TIY, DHK, DAK, EGK, DYK, QNL, QPL, SPV, NPM, STL, NIL, AGA, NTV, SQA, QQA, NNF, NWL, NMA, NNA, NHN, NFH, ELR, NYM, ERA, NFS, SRD, NMM, NNL, NNQ, NNS, QDR, SDK, NNI, AQH, ANA, QNH, QWG, AWA, QWA, EQK, SHS, QFA, SFA, TFA, AYG, EKQ, ERN, EMR, EFR, AYM, EAR, EMK, EYK, EWK, QPI, QPF, NPL, SPI, SPL, NPI, SIV, SLL, SSA, AEM, TML, TLL, SML, TYL, QDM, NSN, AQL, NAT, SQQ, QTI, NAH, SQL, STH, NTT, AEK, ANV, QGL, AGL, QMV, STM, NOM, STI, TQL, SMM, SKE, SQT, SYL, ASL, SLV, NAM, TTV, TPI, TPL, ATL, DNK, DSR, ANL, SHL, SNL, TNL, QNI, QAL, ANI, QNM, QYM, SQI, SNI, QNV, SHV, SHM, QMM, ANM, AHM, SQM, NAL, AQT, EKL, NNM, TNM, SNM, QQI, TQV, TQI, THM, SQV, AAV, THL, AQI, NNV, INN, TNI, SNN, TQM, NAI, TNV, SWT, NMV, NOI, NOL, NMI, NSS, SHF, QNF, SWL, AQM, SYV, TFM, SYM, TYM, ATI, TTI, SLI, ALI, AYI, QLV, TMI, SMI, QFI, NMY, NAY, NMN, NMF, ANF, NWT, QWM, TFT, SWM, TWM, QNW, STF, AFM, NSY, SSY, SNY, THY, TNY, QNY, THE, NSV, ATY, SKD, INF, TWT, QWT, SFS, TFS, DRQ, NYN, DFR, EKG, ENN, AWN, QWQ, TQH, ALV, QHQ, DVR, STS, SQS, AAA, QGQ, QSQ, QAQ, SIH, APY, QGN, AGQ, ASQ, AAQ, SQN, NON, ERM, ALH, NYH, TGH, ATH, QTH, TQT, QQH, AQF, QMH, EKN, ERV, ERS, AFT, EKF, EKT, QSA, NFT, QYH, NYS, TSA, QYY, AYY, AHY, QFN, AWQ, NWQ, QAW, NMS, NWN, QMF, AMH, TSN, TAH, TMH, QFH, NWS, SWN, QRD, QKD, AKE, TSS, AYH, TSH, AHQ, ADK, ADR, ASA, ASS, SNS, QSS, SAH, SMH, QAH, AAH, AHN, QNS, QAS, ANS, ANN, SWA, SWS, TWS, ERI, EKS, QWS, AYS, AFS, SYS, QFS, QYN, QFT, AYT, TNS, TYS, SFT, QNQ, QYS, SYT, ANQ, SWV, SNQ, ANY, TNQ, ATF, SSQ, SSN, TWA, SGQ, SSS, TWY, SMY, TGN, TGY, QWY, SQF, TGF, QTY, TTY, AMY, QMY, ERY, TGQ, SGY, TMF, ALN, ARD, ATN, NAS, SAS, AQN, SYG, SYN, TFN, TMN, AYN, SGN, QNN, DKG, AAN, SMN, SAN, TTF, TQF, SFN, QAF, QFV, AAF, ASF, SAF, SMF, ASY, AFN, TAF, TSY, TAY, QMQ, SMQ, TYV, TGL, QAY, AAY, ADH, EDK, ASN, QYQ, SFQ, TYQ, TWQ, SWQ, AYQ, SYQ, AMV, TMV, TAV, AQV, TYT, TAT, QGI, QQV, SAQ, NAV, SAV, ASV, QSV, QAV, QAI QAM, TAM, DTR, DQR, DMR, NNT, NYT, NMT, NST, ATT, SMV, SNT, QNT, AAT, SAM, SAL, QGV, QST, QYT, ASM, TSM, AMT, TST, ANH, SNV, TNT, ANT, SST, AST, TAI, DAR, SSM, TAL, NSM, TSL, SNF, TSI, TSV, TDK, SAT, SAI, QSL, QSI, ASI, SSL, SSV, SSI, AGI, QGM, TGM, SGM, SGL, SGV, ARL, NWE, SFL, TPY, DRP, DTK, DMK, SEY, SME, EAI, AHD, ADN, EFQ, EMH, SGE, DNQ, DNI, QLY, DGT, EGN, DYN, EWV, ESM, EAL, ASD, DKA, ADF, ADY, SLY, AEY, ETY, EAY, TGA, AVM, TPT, SVI, QSH, TTQ, QTN, TLH, AWY, SLT, AWV, QQY, NFV, SFY, TYY, SHA, TEK, QFK, NNY, EPR, NIT, NHI, NTY, SHY, NFM, NFN, NDK, NER, SER, NDR, QDK, SDR, QNA, TNA, THT, SNH, AYA, NFA, AFA, AHA, QHA, EYR, ENR, EHK, QVL, SVL, NIV, NVI, ADM, SAA, SQH, NQV, ALL, ATM, QYA, STT, DGR, NHT, SHT, QHT, TTL, NHM, QHL, DNR, QHM, QFM, QMI, AHV, THI, AHT, TNH, NHV, THV, SHI, ATV, TWI, NYV, TYI, DYR, SFI, NQF, NWM, NSL, NSP, TQY, DRS, ASP, TKD, QWI, QWN, TWN, QWV, THN, TTM, TQQ, NMQ, TYA, STQ, STN, AGN, EKH, ERT, TFH, SFH, AFH, ENK, NYQ, SFM, QGS, AHF, QLT, QHY, AQY, TRD, ERH, QWH, TYH, TRE, TAQ, AER, TDR, ASH, EKI, AFV, TYN, ALT, NGN, SYY, EWN, NGM, NAN, TAS, TFV, SFV, QAN, SGT, SAY, AFQ, TFQ, QFQ, EFK, SYH, QHV, AMM, THQ, AAI, AAM, NSQ, QAT, TSQ, SHQ, QSM, AGV, AGT, TGT, TGI, AVI, TGV, SGI, ESP, SDA, TPN, EAW, NEQ, DSN, DIK, DNA, QES, SLQ, EKD, SDH, SSF, TET, QDI, DSA, DNM, DKT, DGM, SQY, TLT, QEH, DKQ, TGE, DNS, SDV, SNE, EGT, EGV, AAP, AHG, TWK, TQA, TVT, APQ, SVT, NGT, SSH, NLV, AGY, QFF, QYF, NWV, EKA, EKY, SMG, NAG, ASG, TFI, ELK, SEK, QTL, NML, DHS, ETR, NQQ, QWL, ANP, AHH, NEK, NNN, SNA, EWR, ESR, SHN, SFG, SYA, APV, QPV, SPT, TPQ, QVI, TPV, AVL, SEM, NDM, API, QLM, QTM, QQL, QHI, NFL, AYV, NKE, TIM, QYV, SWI, DRN, AWI, SMA, NRE, QTS, QVV, QQT, QMA, QQS, QAA, ATQ, ERL, TGS, QQQ, AQQ, QHF, TAN, QSY, QSN, NGQ, TWH, TMS, QMS, TQN, TMT, QTV, THS, TTT, SMT, QMT, SMR, ADA, AAE, EPY, EYL, NWI, TSP, DGQ, NDH, QFD, EHL, ERD, DAF, ENI, ENH, QQW, AGM, ENL, EAF, EYV, ENT, SSE, AGE, QEY, DRH, NIQ, SVV, SVM, QPT, TVL, QTQ, SVQ, TIQ, NLQ, SIQ, AWL, EHH, NGY, NHF, NSA, NAA, NTH, QLI, NWH, QRE, AML, EVR, SNP, NHS, THH, NOS, TTS, AHS, EKV, NFQ, APL, QPM, NPV, SIL, TII, STV, QEL, TFL, SIT, NAQ, TMM, AAL, QQM, AMI, QFL, AHI, SYI, SSP, NVV, TIN, TAA, TQS, TMA, ALA, QQN, NSH, TTH, SRE, ERQ, QHS, SWH, SEV, AVT, ALQ, DKH, AMQ, ARE, TWV, NGL, NGV, TMQ, TLV, EER and QTT.

In some embodiments, X₁X₂X₃ is selected from the group consisting of: ANY, SNL AAL AAM, ANT, AST, AYQ, EHK, ENK, ENR, SFQ, SSI, TAY, TDK, TNT, AAF, AAL, AAY, ADK, AFA, ANF, ANI, ANQ, ANS, AQM, ARE, ASV, AYH, AYT, EMK, EWK, NNM, QAF, QAI, QAM, QAT, QAY, QFT, QGM, QHL, QNF, QNQ, QNS, QNT, QNV, QNY, SAH, SAI, SAL, SFT, SFV, SHI, SHV, SMM, SNF, SNM, SNN, SNQ, SNV, SNY, SQL SQV, SSL, SWQ, SWS, SYL, SYM, SYN, SYQ, TAM, TAT, TDR, TFM, THV, TNF, TNH, TNI, TNM, TNQ, TNV, TSY, TWA, and TYM.

In some embodiments, X₁X₂X₃ is selected from the group consisting of: DMK, ATD, EEK, QMD, EFS, ERD, DDR, TDM, SAE, EHS, ENH, SWE, SNE, NNG, QAG, ERG, QSG, QNG, ASG, QFG, AMF, ELR, NFM, NNS, NNI, SDR, EQR, EHR, EWR, EQK, ESR, EKQ, EYR, ENR, EMK, EYK, EHK, EWK, QNI, TNI, TYI, SNY, DRQ, AWI, QWI, DFR, EKG, QYG, QWQ, EKN, EKF, EKT, AFH, ENK, NYS, DKH, AAG, QMF, QFH, QKD, ARE, AHQ, ADK, ADR, AHN, QNS, ANN, SWS, EKS, AFS, QFS, TNQ, TGY, NGL, ARD, AKD, AAF, SMF, AFN, SGT, TGL, ASN, SFQ, AFQ, TFQ, QFQ, EFK, SWQ, AYQ, SYQ, DQR, DMR, ASM, ANT, SHQ, TSL, SNF, QSL, ASI, SGL, SGI, NDA, DHQ, DFK, DTK, DNA, SDS, TDH, DGL, QDV, SDM, DKT, DGM, SQY, DKS, SSD, EMV, TSE, EYV, SSE, EGM, SDF, TTG, QLG, NNP, AGS, QSF, TLM, NGA, AWY, NKD, SWY, TFF, NSG, NGY, QEK, SEK, TEK, AQA, NVL, NHI, TER, NML, NMA, NNA, NHN, ANP, AMS, NER, SER, DKK, NMM, QDK, AHS, TNA, THT, SNH, QNH, ESK, SHS, AYM, SSA, NAT, QLM, QQM, SQI, SNI, QNV, SHV, SHM, NNV, TNN, AHT, TNH, NHV, TQM, THV, SHI, NYL, NMV, NFL, AYV, NQI, NOL, NMI, NSS, SHF, SYM, NYV, AYI, SWI, QFI, ANF, TFT, TWM, QNW, NSY, THY, TNY, NYN, AWN, SMA, NSH, TGH, SFH, SWH, SFM, NFT, QYH, QGS, TGS, TSA, QQQ, QYY, AMQ, TAN, AWH, NMS, TSN, TAH, TMH, QWH, NWS, SWN, QRD, TYH, AKE, TAQ, AER, AYH, ASA, SNS, QSS, SAH, SMH, ASH, QAS, ANS, SYS, QYN, TYN, TNS, SFT, ANQ, SGS, SGH, SSS, TWY, QTY, SYY, NGM, TMS, SYG, TQN, SYN, TEN, TMN, AYN, SGN, QNN, SMN, SAN, QAF, ASF, ASY, TAS, TAF, TFV, SMQ, SFV, TMT, QAN, SAY, EDK, AMV, QHV, THS, TAT, QGI, QQV, NAV, SAV, ASV, QSV, QAV, AAI, AAM, QAM, TAM, DTR, NNT, NYT, NMT, ATT, QTT, TTT, QAT, SMV, SNT, QNT, AAT, SAM, SAL, QGV, QST, QYT, TSM, ANH, SNV, TSQ, INT, SST, AST, TAI, TSI, TSV, TDK, SAT, QSM, SAI, QSI, SSV, SSI, AGT, TGT, AGI, QGM, TGM, TGV, SGM, SGV, SQE, EHT, EYL, DSG, SFE, QND, TQA, AWV, NFV, TSG, SSG, DRG, ANW, TFI, TSF, QNL, STL, TWL, NNF, NND, NYM, NFI, ETR, SNP, EIR, NFS, NNH, NNL, AHL, NNQ, NDR, NNN, QDR, SDK, SNA, AFG, NYA, QHA, QFA, SHN, NFQ, SYA, TFA, EMR, EFR, EAR, NOM, TTL, TQL, SMM, SKE, SYL, ASL, SLV, TTV, AAL, NHL, NHM, QYL, QHL, ATL, DNR, DSR, ANL, SHL, SNL, TNL, QAL, AMI, ANI, QNM, QYM, QHM, QFM, QMM, QMI, ANM, AHM, SQM, AQT, EKL, NNM, TNM, SNM, QQI, TQV, TQI, THI, QHI, THL, SNN, TNV, SWL, AQM, SYV, TFM, TYM, TWI, AFI, ATI, DYR, QLV, TMI, SYI, SFI, NWM, NWT, NSL, STF, SNW, AFM, SSY, NSV, TNF, NAF, TFS, QWV, ERT, TQT, AFT, NYQ, ERL, NRD, TRD, AWQ, TDR, TSH, ASS, AYT, TYS, QYS, SYT, SWV, SNQ, SMS, NAS, SAS, QFV, SAF, TWQ, TAV, AMM, TYT, QAI, TST, DAR, SSM, TAL, NES, DWK, DLR, DIR, SDT, EAN, ETH, TYD, SYE, EAM, TND, AYD, TSP, DRT, NDH, SSF, EFH, DNQ, DSH, DNT, SDI, DKQ, EWT, EAL, EAS, ESL, ASD, EAH, ASE, EAF, ENT, SIY, QDY, APG, AIG, SSW, NPG, ERP, TVI, QSH, SLS, EDR, AWL, SLH, EQH, EHH, NWV, EKY, STY, DRI, EYH, DRV, SPY, NNY, DWR, ERA, QRE, EVR, SRD, AWA, SFA, QTI, SHT, THM, AQI, AHI, SWT, ATV, TTI, SMI, QYV, NMY, SWM, QNY, TWT, QWT, TQY, NRE, QTS, QHQ, DVR, AAA, TYA, EKH, ALH, AWT, ATH, SRE, TFH, QMH, ERV, ERS, QHY, ALQ, QSY, ERH, TRE, TSS, QAH, AAH, SWA, TWS, QWS, AYS, AFV, QNQ, ANY, ALT, SMY, TWV, TTY, AMY, QMY, ERY, TWH, TSY, TAY, TYV, QAY, AAY, QYQ, TYQ, SYH, AQV, EER, THQ, and SMT.

In some embodiments, X₁X₂X₃ is selected from the group consisting of: ADR, ASI, EFK, EHK, EWK, SYQ, AAF, AAT, AAY, AFI, AFQ, AGI, AGT, AHI, ANH, ANM, ANN, AQI, ASA, ASH, AST, ASV, AWT, AYQ, AYT, DAR, DMK, DQR, DVR, EAR, EFR, EMK, EMR, EQK, ERA, ERS, ESR, NDA, NDR, NMI, NMV, NNM, NNN, NYL, NYM, NYN, NYQ, NYV, QAM, QFQ, QFV, QGV, QNH, QNI, QNM, QQV, QSF, QSY, SAI, SAM, SAS, SDR, SFQ, SGH, SGM, SGT, SGV, SHL, SHM, SHQ, SHV, SMH, SNA, SNE, SNF, SNI, SNQ, SNT, SRE, SST, SWQ, SWT, SYG, SYY, TFQ, THL, THQ, THV, TMI, TNL, TNS, TSH, TSQ, and TWQ.

In some embodiments, the targeting peptide comprises a 7-mer peptide (RGDX₇X₈X₉X₁₀) having an amino acid sequence selected from SEQ ID NOs.: 238-44858. In this case, X₁X₂X₃ can be the amino acids at the corresponding positions of the reference AAV capsid. For example, X₁X₂X₃ can be the naturally occurring amino acids at the corresponding positions of the reference AAV capsid. In some embodiments, the targeting peptide comprises a 7-mer peptide (RGDX₇X₈X₉X₁₀) having an amino acid sequence with at least 90%, 95%, 98%, 99% sequence identity to the amino acid sequence selected from SEQ ID NOs.: 238-44858.

In some embodiments, the modified AAV capsid protein comprises targeting peptide including a 7-mer peptide (RGDX₇X₈X₉X₁₀) having an amino acid sequence selected from SEQ ID NOs.: 238-247. In this case, X₁X₂X₃ can be the amino acids at the corresponding positions of the reference AAV capsid. In some embodiments, X₁X₂X₃ has a sequence that is different from the sequence at the corresponding positions of the reference AAV capsid. In some embodiments, any one of the corresponding positions X₁, X₂, X₃ of the reference AAV can be substituted with any amino acid. In some embodiments, the 7-mer has a sequence selected from SEQ ID NOs.: 238-337. In some embodiments, the 7-mer has a sequence selected from SEQ ID NOs.: 238-437. In some embodiments, the 7-mer has a sequence selected from SEQ ID NOs.: 238-537. In some embodiments, the 7-mer has a sequence selected from SEQ ID NOs.: 238-637. In some embodiments, the 7-mer has a sequence selected from SEQ ID NOs.: 238-737. In some embodiments, the 7-mer has a sequence selected from SEQ ID NOs.: 238-837. In some embodiments, the 7-mer has a sequence selected from SEQ ID NOs.: 238-937. In some embodiments, the 7-mer has a sequence selected from SEQ ID NOs.: 238-1037.

In some embodiments, the targeting peptide comprises a 7-mer peptide (RGD X₇ X₈ X₉ X₁₀) having an amino acid sequence selected from SEQ ID NOs.: 3881, 12092, 14601, 15342, 21498, and 31396.

In some embodiments, the targeting peptide comprises a 7-mer peptide (RGD X₇ X₈ X₉ X₁₀) having an amino acid sequence of SEQ ID NO: 238.

In some embodiments, the targeting peptide comprises an amino acid sequence selected from SEQ ID NOs.: 44859-44883, 44911, 44912, 44913, and 44918-44919. In some embodiments, the targeting peptide comprises an amino acid sequence selected from SEQ ID NOs.: 44859-44878. In some embodiments, the targeting peptide comprises an amino acid sequence of 44859. In some embodiments, the targeting peptide comprises an amino acid sequence of 44860. In some embodiments, the targeting peptide comprises an amino acid sequence of 44861. In some embodiments, the targeting peptide comprises an amino acid sequence of 44862. In some embodiments, the targeting peptide comprises an amino acid sequence of 44863. In some embodiments, the targeting peptide comprises an amino acid sequence of 44864. In some embodiments, the targeting peptide comprises an amino acid sequence of 44865. In some embodiments, the targeting peptide comprises an amino acid sequence of 44866. In some embodiments, the targeting peptide comprises an amino acid sequence of 44867. In some embodiments, the targeting peptide comprises an amino acid sequence of 44868. In some embodiments, the targeting peptide comprises an amino acid sequence of 44869. In some embodiments, the targeting peptide comprises an amino acid sequence of 44870. In some embodiments, the targeting peptide comprises an amino acid sequence of 44871. In some embodiments, the targeting peptide comprises an amino acid sequence of 44872. In some embodiments, the targeting peptide comprises an amino acid sequence of 44873. In some embodiments, the targeting peptide comprises an amino acid sequence of 44874. In some embodiments, the targeting peptide comprises an amino acid sequence of 44875. In some embodiments, the targeting peptide comprises an amino acid sequence of 44876. In some embodiments, the targeting peptide comprises an amino acid sequence of 44877. In some embodiments, the targeting peptide comprises an amino acid sequence of 44878. In some embodiments, the targeting peptide comprises an amino acid sequence of 44879. In some embodiments, the targeting peptide comprises an amino acid sequence of 44880. In some embodiments, the targeting peptide comprises an amino acid sequence of 44881. In some embodiments, the targeting peptide comprises an amino acid sequence of 44882. In some embodiments, the targeting peptide comprises an amino acid sequence of 44883. In some embodiments, the targeting peptide comprises an amino acid sequence of 44911. In some embodiments, the targeting peptide comprises an amino acid sequence of 44912. In some embodiments, the targeting peptide comprises an amino acid sequence of 44913. In some embodiments, the targeting peptide comprises an amino acid sequence of 44918. In some embodiments, the targeting peptide comprises an amino acid sequence of 44919. In some embodiments, the targeting peptide does not comprise a peptide selected from: SEQ ID NOs: 44855 and 3000. In some embodiments, the targeting peptide does not comprise a peptide selected from: SEQ ID NOs: 44880 and 44910. In certain embodiments, the targeting peptide comprises an amino acid sequence having at least 90%, 95%, 98%, 99% sequence identity to the amino acid sequence selected from SEQ ID NOs.: 44859-44883, 44911, 44912, 44913, and 44918-44919.

In some embodiments, the targeting peptide comprises an amino acid sequence selected from SEQ ID NOs.: 44864-44867, 44879-44883, 44911, 44912, 44913, and 44918-44919. In certain embodiments, the targeting peptide comprises an amino acid sequence having at least 90%, 95%, 98%, 99% sequence identity to the amino acid sequence selected from SEQ ID NOs.: 44864-44867, 44879-44883, 44911, 44912, 44913, and 44918-44919. In some embodiments, the targeting peptide comprises an amino acid sequence of SEQ ID NO: 44864. In some embodiments, the targeting peptide comprises an amino acid sequence of SEQ ID NO: 44865. In some embodiments, the targeting peptide comprises an amino acid sequence of SEQ ID NO: 44866. In some embodiments, the targeting peptide comprises an amino acid sequence of SEQ ID NO: 44867. In some embodiments, the targeting peptide comprises an amino acid sequence of SEQ ID NO: 44879. In some embodiments, the targeting peptide comprises an amino acid sequence of SEQ ID NO: 44880. In some embodiments, the targeting peptide comprises an amino acid sequence of SEQ ID NO: 44881. In some embodiments, the targeting peptide comprises an amino acid sequence of SEQ ID NO: 44882. In some embodiments, the targeting peptide comprises an amino acid sequence of SEQ ID NO: 44883. In some embodiments, the targeting peptide comprises an amino acid sequence of SEQ ID NO: 44911. In some embodiments, the targeting peptide comprises an amino acid sequence of SEQ ID NO: 44912. In some embodiments, the targeting peptide comprises an amino acid sequence of SEQ ID NO: 44913. In some embodiments, the targeting peptide comprises an amino acid sequence of SEQ ID NO: 44918. In some embodiments, the targeting peptide comprises an amino acid sequence of SEQ ID NO: 44919.

In some embodiments, the targeting peptide comprises a sequence X₇ is selected from R, F, H, L, Q, R, and Y. In some embodiments, X₇ is R. In some embodiments, X₇ is Y or H In some embodiments, X₇ is F or Y.

In some embodiments, X₈ is selected from S, G, D, I, L, N, Q, T, and V. In some embodiments. X₈ is S or G. In some embodiments, X₈ is S. In some embodiments, X₈ is G. In some embodiments, X₈ is T, G or S. In some embodiments, X₈ is N or Q. In some embodiments, X₉ is selected from S or V.

In some embodiments, X₉ is selected from any of the amino acids. In some embodiments, X₉ is selected from V, S, N, G, Q, L, T, and Y. In some embodiments, X₉ is selected from V or Q. In some embodiments, X₉ is selected from N or S.

In some embodiments, X₁₀ is selected from I, V, S, L, M, R, T, and Q. In some embodiments, X₁₀ is I or V. In some embodiments, X₁₀ is I. X₁₀ is V. In some embodiments, X₁₀ is V, L or M. In some embodiments, X₁₀ is T or L.

In some embodiments, the targeting peptide comprises a sequence of RGDRX₈X₉X₁₀. In some embodiments, X₈ is S or G. In some embodiments, X₈ is S. In some embodiments, X₈ is G. In some embodiments, X₉ is selected from any amino acid residue. In some embodiments, X₉ is selected from V, S, N, G, and Q. In some embodiments, X₉ is V. In some embodiments, X₉ is S. In some embodiments, X₉ is N. In some embodiments, X₉ is G. In some embodiments, X₉ is Q. In some embodiments, X₁₀ is selected from I, V, S, L and Q. In some embodiments, X₁₀ is I. In some embodiments, X₁₀ is V. In some embodiments, X₁₀ is S. In some embodiments, X₁₀ is L. In some embodiments, X₁₀ is Q. In some embodiments, X₁₀ is I or V.

In some embodiments, the targeting peptide has a sequence of X₁X₂X₃RGDRGVV (SEQ ID NO: 98928), X₁X₂X₃RGDRSVV (SEQ ID NO: 98931), X₁X₂X₃RGDRGQI (SEQ ID NO: 98927), X₁X₂X₃RGDRSQT (SEQ ID NO: 98930), X₁X₂X₃RGDRQGI (SEQ ID NO: 98929), X₁X₂X₃RGDFQNT (SEQ ID NO: 98934), X₁X₂X₃RGDHGVL (SEQ ID NO: 98938), X₁X₂X₃RGDYTSV (SEQ ID NO: 98941), X₁X₂X₃RGDYTSM (SEQ ID NO: 98942), X₁X₂X₃RGDLTVT (SEQ ID NO: 98935), X₁X₂X₃RGDFNNT (SEQ ID NO: 98943), X₁X₂X₃RGDYSSV (SEQ ID NO: 98937), X₁X₂X₃RGDHVNL (SEQ ID NO: 98924), X₁X₂X₃RGDQSTL (SEQ ID NO: 98926), X₁X₂X₃RGDLIGR (SEQ ID NO: 98925), X₁X₂X₃RGDFNNL (SEQ ID NO: 98933), X₁X₂X₃RGDLLLS (SEQ ID NO: 98932), X₁X₂X₃RGDYNSL (SEQ ID NO: 98940), X₁X₂X₃RGDRDYL (SEQ ID NO: 98939), or X₁X₂X₃RGDYVGL (SEQ ID NO: 98936).

In some embodiments, the targeting peptide has a sequence of X₁X₂X₃RGDRGVV (SEQ ID NO: 98928), X₁X₂X₃RGDRSVV (SEQ ID NO: 98931) or X₁X₂X₃RGDRGQI (SEQ ID NO: 98927).

In some embodiments, the targeting peptide has a sequence of X₁X₂X₃RGDYTSV (SEQ ID NO: 98941), X₁X₂X₃RGDYTSM (SEQ ID NO: 98942), X₁X₂X₃RGDRGVV (SEQ ID NO: 98928), X₁X₂X₃RGDRSVV (SEQ ID NO: 98931), X₁X₂X₃RGDYSSV (SEQ ID NO: 98937), or X₁X₂X₃RGDHGVL (SEQ ID NO: 98938).

In some embodiments, the targeting peptide has a sequence of X₁X₂X₃RGDFQNT (SEQ ID NO: 98934), X₁X₂X₃RGDHGVL (SEQ ID NO: 98938), X₁X₂X₃RGDLIGR (SEQ ID NO: 98925), X₁X₂X₃RGDRGQI (SEQ ID NO: 98927), X₁X₂X₃RGDRGVV (SEQ ID NO: 98928), X₁X₂X₃RGDYTSM (SEQ ID NO: 98942) or X₁X₂X₃RGDYTSV (SEQ ID NO: 98941).

In some embodiments, X₁X₂X₃ is selected from the group consisting of EFK, AAY, DQK, QVY, DKL, DNV, ENF, EWK, QNV, and TFM.

In certain embodiments, the modified sequence comprises a 7-mer peptide selected from: RGDRSX₉I, RGDRGX₉I, RGDRSX₉V, or RGDRGX₉V.

In some embodiments, the targeting peptide has an amino acid sequence selected from X₁X₂X₃RGDRGQI (SEQ ID NO: 98927), X₁X₂X₃RGDRSVV (SEQ ID NO: 98931) or X₁X₂X₃RGDRGVV (SEQ ID NO: 98928).

In some embodiments, X₁X₂X₃ is selected from the group consisting of DAV, DKW, EAY, AEY, DFV, DKF, DKI, DKL, DNV, DNY, DSL, DSV, EFI, SEF, SEY, SLY, ADF, ADY, ALY, AVF, DAF, DAL, DAM, DAT, DHV, DIV, DKA, DKM, DKT, DKV, DKY, DMI, DNF, DNL, DQT, DSI, DVY, DYN, DYV, EAT, EAW, EFV, EGL, EIY, EMF, EMY, ENF, EPF, EPY, EQY, ESY, ETF, EWI, EWT, EYI, EYV, NEM, QDF, QDY, QEY, QLY, QND, QVF, QVY, SDL, SDV, SEH, SII, SIY, SSL, SVY, SWD, SWE, TDF, TDV, TDY, TEF, TEY, TIY, TLY, and TWQ.

In some embodiments, X₁X₂X₃ is selected from the group consisting of: ETI, DQN, DLL, EKW, DNN, EYS, or TVF.

In some embodiments, X₇ is selected from Y and H; X₈ is selected from T, G and S; X₉ is selected from S and V; X₁₀ is selected from V, L and M

In some embodiments, the targeting peptide has an amino acid sequence selected from: X₁X₂X₃RGDHGVL (SEQ ID NO: 98938), X₁X₂X₃RGDYSSV (SEQ ID NO: 98937), X₁X₂X₃RGDYTSM (SEQ ID NO: 98942) or X₁X₂X₃RGDYTSV (SEQ ID NO: 98941).

In some embodiments, X₁X₂X₃ is selected from the group consisting of: ANY, SNI, AAI, AAM, ANT, AST, AYQ, EHK, ENK, ENR, SFQ, SSI, TAY, TDK, TNT, AAF, AAL, AAY, ADK, AFA, ANF, ANI, ANQ, ANS, AQM, ARE, ASV, AYH, AYT, EMK, EWK, NNM, QAF, QAI, QAM, QAT, QAY, QFT, QGM, QHL, QNF, QNQ, QNS, QNT, QNV, QNY, SAH, SAI, SAL, SFT, SFV, SHI, SHV, SMM, SNF, SNM, SNN, SNQ, SNV, SNY, SQI, SQV, SSL, SWQ, SWS, SYI, SYM, SYN, SYQ, TAM, TAT, TDR, TFM, THV, TNF, TNH, TNI, TNM, TNQ, TNV, TSY, TWA, and TYM.

In some embodiments, X₁X₂X₃ is selected from TVF, APM, AQI, TTS, NQF, NFL or SMN.

In some embodiments, X₇ is selected from F and Y; X₈ is selected from N and Q; X₉ is selected from N and S; X₁₀ is selected from T and L.

In some embodiments, the targeting peptide has an amino acid sequence selected from: X₁X₂X₃RGDFNNT (SEQ ID NO: 98943), X₁X₂X₃RGDENNL (SEQ ID NO: 98933), X₁X₂X₃RGDFQNT (SEQ ID NO: 98934) or X₁X₂X₃RGDYNSL (SEQ ID NO: 98940).

In some embodiments, X₁X₂X₃ is selected from the group consisting of: ADR, ASI, EFK, EHK, EWK, SYQ, AAF, AAT, AAY, AFI, AFQ, AGI, AGT, AHI, ANH, ANM, ANN, AQI, ASA, ASH, AST, ASV, AWT, AYQ, AYT, DAR, DMK, DQR, DVR, EAR, EFR, EMK, EMR, EQK, ERA, ERS, ESR, NDA, NDR, NMI, NMV, NNM, NNN, NYL, NYM, NYN, NYQ, NYV, QAM, QFQ, QFV, QGV, QNH, QNI, QNM, QQV, QSF, QSY, SAI, SAM, SAS, SDR, SFQ, SGH, SGM, SGT, SGV, SHL, SHM, SHQ, SHV, SMH, SNA, SNE, SNF, SNI, SNQ, SNT, SRE, SST, SWQ, SWT, SYG, SYY, TFQ, THL, THQ, THV, TMI, TNL, TNS, TSH, TSQ, and TWQ.

In some embodiments, X₁X₂X₃ is selected from the group consisting of: TVF, APM, AQI, TTS, NOF, NFL or SMN.

In certain embodiments, the modified sequence comprises a 7-mer peptide selected from: RGDRGVX₁₀ (SEQ ID NO: 157058), RGDRGSX₁₀ (SEQ ID NO: 157059), RGDRGNX₁₀ (SEQ ID NO: 157060), RGDRGGX₁₀ (SEQ ID NO: 157061), RGDRGQX₁₀ (SEQ ID NO: 157062), RGDRGX₉V (SEQ ID NO: 157063), RGDRGX₉I (SEQ ID NO: 157064), RGDRGX₉S (SEQ ID NO: 157065), RGDRGX₉L (SEQ ID NO: 157066). RGDROX₉Q (SEQ ID NO: 157067), RGDHX₈X₉L (SEQ ID NO. 157068). RGDRX₈X₉I (SEQ ID NO: 157069). RGDRX₈X₉V (SEQ ID NO: 157070). RGDRX₈X₉L (SEQ ID NO: 157071). RGDYX₈X₉L (SEQ ID NO: 157072), RGDYX₈X₉V (SEQ ID NO: 157073), RGDYX₈X₉M (SEQ ID NO: 157074), and RGDLX₈X₉T (SEQ ID NO: 157075).

In certain embodiments, the targeting peptide comprises a sequence of H1 (RGDLIGR (SEQ ID NO: 1422)) or a sequence of H2 (RGDQSTL (SEQ ID NO: 3052)). In some embodiments, the targeting peptide comprises a sequence of H1 (RGDLIGR (SEQ ID NO: 1422)) targeting peptide located in VR VIII between amino acids at positions 588 and 589 or a sequence of H2 (RGDQSTL (SEQ ID NO: 3052)) targeting peptide located in VR VIII between amino acids at positions 588 and 589. In such embodiments, a modified AAV capsid protein comprising an H1 or H2 targeting peptide have increased tissue enrichment (tropism) in cardiac muscle tissue as compared to skeletal muscle tissue.

In certain embodiments, the targeting peptide comprises a sequence of S1 (RGDISRT (SEQ ID NO: 263)) targeting peptide located in VR VIII or a sequence of S2 (RGDRSQT (SEQ ID NO: 251)). In some embodiments, the targeting peptide comprises a sequence of S1 (RGDISRT (SEQ ID NO. 263)) targeting peptide located in VR VIII between amino acids 588 and 589 or a sequence of S2 (RGDRSQT (SEQ ID NO: 251) targeting peptide located in VR VIII between amino acids at positions 588 and 589. In such embodiments, a modified AAV capsid protein comprising an S1 or S2 targeting peptide have increased tissue enrichment (tropism) in skeletal tissue as compared to cardiac muscle tissue.

In certain embodiments, the modified sequence does not comprise a 7-mer peptide selected from: RGDRMVF, RGDRTVI, SRGDRPM, and ISLRGDR. In some embodiments, the modified sequence does not comprise a 7-mer peptide having an amino acid sequence of SEQ ID NO: 1 (RGDLLLS). In certain embodiments, the modified sequence does not comprise a 7-mer peptide of RGDRMVF. In certain embodiments, the modified sequence does not comprise a 7-mer peptide of RGDRTVI. In certain embodiments, the modified sequence does not comprise a 7-mer peptide selected from SRGDRPM, or ISLRGDR. In some embodiments, the modified sequence does not comprise a 7-mer peptide selected from: RGDRMVF, RGDRTVI, SRGDRPM, and ISLRGDR. In some embodiments, the modified sequence does not comprise a 7-mer peptide of X₁SLRGDR, where X₁ is any amino acid residue. In some embodiments, the modified sequence does not comprise a 7-mer peptide of X₁X₂LRGDR, where X₁ and X₂ are independently any amino acid residue.

In some embodiments, the targeting peptide has a sequence selected from: X₁X₂X₃RGDHVNL (SEQ ID NO: 98924); X₁X₂X₃RGDLIGR (SEQ ID NO: 98925); X₁X₂X₃RGDQSTL (SEQ ID NO: 98926); X₁X₂X₃RGDRGQI (SEQ ID NO: 98927); X₁X₂X₃RGDRGVV (SEQ ID NO. 98928); X₁X₂X₃RGDRQGI (SEQ ID NO: 98929); X₁X₂X₃RGDRSQT (SEQ ID NO: 98930); X₁X₂X₃RGDRSVV (SEQ ID NO: 98931); X₁X₂X₃RGDLLLS (SEQ ID NO: 98932); X₁X₂X₃RGDFNNL (SEQ ID NO: 98933); X₁X₂X₃RGDFQNT (SEQ ID NO: 98934); X₁X₂X₃RGDLTVT (SEQ ID NO: 98935); X₁X₂X₃RGDYVGL (SEQ ID NO: 98936); X₁X₂X₃RGDYSSV (SEQ ID NO: 98937); X₁X₂X₃RGDHGVL (SEQ ID NO: 98938); X₁X₂X₃RGDRDYL (SEQ ID NO: 98939); X₁X₂X₃RGDYNSL (SEQ ID NO: 98940); X₁X₂X₃RGDYTSV (SEQ ID NO: 98941); X₁X₂X₃RGDYTSM (SEQ ID NO: 98942); and X₁X₂X₃RGDFNNT (SEQ ID NO. 98943). In some embodiments, X₁ is selected from S, E, A, D, N, Q, or T. In some embodiments, X₁ is selected from S or E. In some embodiments, X₁ is S. In some embodiments, X₁ is E. In some embodiments, X₂ is selected from N, A, D, E, F, G, H, I, K, L, M, N, P, Q, R, S, T, V, W, or Y. In some embodiments, X₂ is selected from N or A. In some embodiments, X₂ is N. In some embodiments, X₂ is A. In some embodiments, X₃ is selected from R, Q, A, D, E, F, G, H, I, K, L, M, N, P, S, T, V, W, or Y In some embodiments, X₃ is selected from R or Q. In some embodiments, X₃ is R. In some embodiments, X₃ is Q. In some embodiments, X₂ is N. and X₃ is R. In some embodiments, X₁ is E; X₂ is N; and X₃ is R. In some embodiments, X₁ is S; X₂ is N; and X₃ is R.

In some embodiments, the modified AAV capsid protein further includes one or more mutations outside of the VRVIII. The mutant outside of VRVIII can comprise (1) an alanine (A) or glycine (G) amino acid residue at an amino acid position corresponding to position 266 in Anc80 VP1; and/or (2) a lysine (K) or arginine (R) amino acid residue at an amino acid position corresponding to position 168 in Anc80 VP1. The modified AAV capsid protein can comprise an alanine (A) amino acid residue at an amino acid position 267 and a threonine (T) amino acid residue at an amino acid position 269 in AAV9 VP1.

In some embodiments, the modified AAV capsid protein further includes one or more deletions or substitutions within the VRVIII. In some embodiments, the modified AAV capsid protein further includes one or more deletions or substitutions between positions 565 and 595 of the reference capsid protein. In some embodiments, the modified AAV capsid protein further includes one or more deletions or substitutions within the VRVIII, followed by an insertion of the targeting peptide. In some embodiments, the modified AAV capsid protein further includes one or more substitutions within the VRVIII, followed by an insertion of the targeting peptide. In some embodiments, the modified AAV capsid protein further includes one or more substitutions within the VRVIII. In some embodiments, the modified AAV capsid protein further includes one or more substitutions between positions 565 and 595 of the reference capsid protein. In some embodiments, the modified AAV capsid protein further includes one or more substitutions within the VRVIII, followed by an insertion of the targeting peptide.

In some embodiments, the targeting peptide can enhance targeting of an AAV to a brain, muscle, spinal cord, eye, liver, heart, muscle, or other organ. In some embodiments, the targeting peptide can decrease targeting of an AAV to a brain, muscle, spinal cord, eye, liver, heart, muscle, or other organ.

Sequences of exemplary targeting peptides that can be used various embodiments of the present disclosure are provided in SEQ ID Nos: 1-53, 153-157, 160-162, 238-44858, 3881, 12092, 14601, 15342, 21498, 31396, 44859-44883, 44910, 44911, 44912, 44913, 44918, 44919, and 48391-157057.

In some embodiments, the targeting peptide comprises at least 90%, at least 95%, at least 98%, at least 99%, or at least 100% sequence identity to the amino acid sequence of ENRRGDYNSL (SEQ ID NO: 44859). In some embodiments, the targeting peptide comprises at least 90%, at least 95%, at least 98%, at least 99%, or at least 100% sequence identity to the amino acid sequence of ENRRGDYNNL (SEQ ID NO: 44860). In some embodiments, the targeting peptide comprises at least 90%, at least 95%, at least 98%, at least 99%, or at least 100% sequence identity to the amino acid sequence of ENRRGDYNST (SEQ ID NO: 44861). In some embodiments, the targeting peptide comprises at least 90%, at least 95%, at least 98%, at least 99%, or at least 100% sequence identity to the amino acid sequence of ENRRGDYNNT (SEQ ID NO. 44862). In some embodiments, the targeting peptide comprises at least 90%, at least 95%, at least 98%, at least 99%, or at least 100% sequence identity to the amino acid sequence of ENRRGDFNSL (SEQ ID NO: 44863). In some embodiments, the targeting peptide comprises at least 90%, at least 95%, at least 98%, at least 99%, or at least 100% sequence identity to the amino acid sequence of ENRRGDFNNL (SEQ ID NO: 44864). In some embodiments, the targeting peptide comprises at least 90%, at least 95%, at least 98%, at least 99%, or at least 100% sequence identity to the amino acid sequence of ENRRGDENST (SEQ ID NO: 44865). In some embodiments, the targeting peptide comprises at least 90%, at least 95%, at least 98%, at least 99%, or at least 100% sequence identity to the amino acid sequence of ENRRGDFQNT (SEQ ID NO: 44866). In some embodiments, the targeting peptide comprises at least 90%, at least 95%, at least 98%, at least 99%, or at least 100% sequence identity to the amino acid sequence of ENRRGDEQNT (SEQ ID NO: 44867). In some embodiments, the targeting peptide comprises at least 90%, at least 95%, at least 98%, at least 99%, or at least 100% sequence identity to the amino acid sequence of ENRRGDQQNT (SEQ ID NO: 44868). In some embodiments, the targeting peptide comprises at least 90%, at least 95%, at least 98%, at least 99%, or at least 100% sequence identity to the amino acid sequence of SNRRGDYNSL (SEQ ID NO: 44869) In some embodiments, the targeting peptide comprises at least 90%, at least 95%, at least 98%, at least 99%, or at least 100% sequence identity to the amino acid sequence of SNRRGDYNNL (SEQ ID NO: 44870). In some embodiments, the targeting peptide comprises at least 90%, at least 95%, at least 98%, at least 99%, or at least 100% sequence identity to the amino acid sequence of SNRRGDYNST (SEQ ID NO: 44871). In some embodiments, the targeting peptide comprises at least 90%, at least 95%, at least 98%, at least 99%, or at least 100% sequence identity to the amino acid sequence of SNRRGDYNNT (SEQ ID NO: 44872) In some embodiments, the targeting peptide comprises at least 90%, at least 95%, at least 98%, at least 99%, or at least 100% sequence identity to the amino acid sequence of SNRRGDFNSL (SEQ ID NO: 44873). In some embodiments, the targeting peptide comprises at least 90%, at least 95%, at least 98%, at least 99%, or at least 100% sequence identity to the amino acid sequence of SNRRGDFNNL (SEQ ID NO: 44874). In some embodiments, the targeting peptide comprises at least 90%, at least 95%, at least 98%, at least 99%, or at least 100% sequence identity to the amino acid sequence of SNRRGDFNST (SEQ ID NO: 44875). In some embodiments, the targeting peptide comprises at least 90%, at least 95%, at least 98%, at least 99%, or at least 100% sequence identity to the amino acid sequence of SNRRGDFQNT (SEQ ID NO: 44876). In some embodiments, the targeting peptide comprises at least 90%, at least 95%, at least 98%, at least 99%, or at least 100% sequence identity to the amino acid sequence of SNRRGDEQNT (SEQ ID NO: 44877). In some embodiments, the targeting peptide comprises at least 90%, at least 95%, at least 98%, at least 99%, or at least 100% sequence identity to the amino acid sequence of SNRRGDQQNT (SEQ ID NO: 44878). In some embodiments, the targeting peptide comprises at least 90%, at least 95%, at least 98%, at least 99%, or at least 100% sequence identity to the amino acid sequence of ENRRGDFNGL (SEQ ID NO: 44879). In some embodiments, the targeting peptide comprises at least 90%, at least 95%, at least 98%, at least 99%, or at least 100% sequence identity to the amino acid sequence of ENRRGDFNNT (SEQ ID NO: 44880) In some embodiments, the targeting peptide comprises at least 90%, at least 95%, at least 98%, at least 99%, or at least 100% sequence identity to the amino acid sequence of SAQRGDFNNT (SEQ ID NO: 44881). In some embodiments, the targeting peptide comprises at least 90%, at least 95%, at least 98%, at least 99%, or at least 100% sequence identity to the amino acid sequence of SAQRGDLLLS (SEQ ID NO: 44882). In some embodiments, the targeting peptide comprises at least 90%, at least 95%, at least 98%, at least 99%, or at least 100% sequence identity to the amino acid sequence of SNRRGDFNNT (SEQ ID NO: 44883). In some embodiments, the targeting peptide comprises at least 90%, at least 95%, at least 98%, at least 99%, or at least 100% sequence identity to the amino acid sequence of (SEQ ID NO: 44911). In some embodiments, the targeting peptide comprises at least 90%, at least 95%, at least 98%, at least 99%, or at least 100% sequence identity to the amino acid sequence of (SEQ ID NO: 44912). In some embodiments, the targeting peptide comprises at least 90%, at least 95%, at least 98%, at least 99%, or at least 100% sequence identity to the amino acid sequence of (SEQ ID NO: 44913). In some embodiments, the targeting peptide comprises at least 90%, at least 95%, at least 98%, at least 99%, or at least 100% sequence identity to the amino acid sequence of (SEQ ID NO: 44918). In some embodiments, the targeting peptide comprises at least 90%, at least 95%, at least 98%, at least 99%, or at least 100% sequence identity to the amino acid sequence of (SEQ ID NO: 44919). In some embodiments, the targeting peptide does not have the amino acid sequence of SEQ ID NO: 44910. In some embodiments, the targeting peptide does not have the amino acid sequence of SEQ ID NO: 44880. In some embodiments, the targeting peptide does not have the amino acid sequence selected from SEQ ID NOs: 44855-44858.

In some embodiments, in the targeting peptide. X₇, X₈, and X₉ are independently selected from L, G, V, and A; and X₇ is S, V, A, G, or L. In some embodiments, X₇ is selected from A, D, E, F, G, H, I, K, L, N, Q, R, S, T, V, and Y. In some embodiments, X₈ is selected from A, D, E, F, G, H, I, K, L, N, Q, R, S, T, V, and Y. In some embodiments, X₉ is selected from A, D, E, F, G, H, I, K, L, N, Q, R, S, T, V, and Y. In some embodiments, X₁₀ is selected from A, D, E, F, G, H, I, K, L, N, Q, R, S, T, V, and Y.

In some embodiments, X₇ is selected from L, Q, D, H, M, P, and K. In some embodiments, X₇ is L. In some embodiments, X₈ is selected from G, V, S, D, M, and N. In some embodiments, X₈ is G. In some embodiments, X₉ is selected from V, M, P, S, and D. In some embodiments, X₉ is V. In some embodiments, X₁₀ is selected from S, N, L, H, and M. In some embodiments, X₁₀ is S.

In some embodiments, X₇ is L. In further embodiments, X₈ is G. In further embodiments, X₉ is L. In further embodiments, X₁₀ is S.

In some embodiments, X₇ is A. In further embodiments, X₈ is V. In further embodiments, X₉ is G. In further embodiments, X₁₀ is V.

In some embodiments, X₇ is L. In further embodiments, X₈ is L. In further embodiments, X₉ is L. In further embodiments, X₁₀ is S.

In some embodiments, X₇ is L; X₈ is selected from G, L and V; X₉ is selected from L and G; and/or X₁₀ is selected from S, V and L. In a further embodiment, X₇ is L, X₈ is G or L, and/or X₁₀ is S. In certain embodiments, X₈ and X₉ is G or L.

In certain embodiments. X₇, X₈, and X₉ are independently selected from L, V, and A; at least two of X₇, X₈, and X₉ are independently L. In some embodiments, X₇, X₈, and X₉ are L. In certain embodiments, X₈ is L.

In some embodiments, the targeting peptide has a sequence of X₁X₂X₃RGDX₇X₈X₉X₁₀, wherein X₇X₈X₉X₁₀ is a quad selected for enhanced targeting to muscle. In some embodiments, the targeting peptide has a sequence of X₁X₂X₃RGDX₇X₈X₉X₁₀, wherein X₇X₈X₉X₁₀ is a quad selected for enhanced targeting to a specific muscle (e.g., biceps, quadriceps, diaphragm, heart).

In some embodiments, the targeting peptide has a sequence of X₁X₂X₃RGDX₇X₈X₉X₁₀, wherein X₇X₈X₉X₁₀ is a quad selected for enhanced targeting to skeletal muscle. In some embodiments, the quad is FNNL, YNSL, RQGI, FONT, YVGL, YSSV, YTSM, RSVV, YTSV, RDYL, or FNNT.

In some embodiments, the targeting peptide has a sequence of X₁X₂X₃RGDX₇X₈X₉X₁₀, wherein X₇X₈X₉X₁₀ is a quad selected for enhanced targeting to cardiac muscle. In some embodiments, the quad is LIGR, QSTL, or RGVV.

In some embodiments, the targeting peptide has a sequence of X₁X₂X₃RGDX₇X₈X₉X₁₀, wherein X₇X₈X₉X₁₀ is a quad selected for enhanced targeting to liver. In some embodiments, the quad is RGVV, RSVV, RGQI, RSQT, RQGI, FONT, HGVL, YTSV, YTSM, LTVT, FNNT, YSSV, or HVNL

In certain embodiments, the targeting peptide comprises the amino acid sequence selected from RGDLRVS (SEQ ID NO: 153), RGDAVGV (SEQ ID NO: 154), RGDFTPTS (SEQ ID NO: 155), RGDLGLS (SEQ ID NO: 156), and RGDMSRE (SEQ ID NO: 157), and/or a sequence comprising at most two, preferably at most one, amino acid substitution compared to one of the aforesaid specific sequences. In certain embodiments, the targeting peptide does not comprise an amino acid sequence selected from RGDLRVS (SEQ ID NO: 153), RGDAVOV (SEQ ID NO: 154), RGDFTPTS (SEQ ID NO: 155), RGDLGLS (SEQ ID NO: 156), and RGDMSRE (SEQ ID NO: 157).

In some embodiments, the targeting peptide comprises a sequence of RGDLLLS (SEQ ID NO: 1).

In some embodiments, the targeting peptide comprises a sequence selected from: SEQ ID NOs.: 238-44858. In some embodiments, the modified AAV capsid protein comprises a targeting peptide comprising an amino acid sequence selected from SEQ ID NOs.: 238-247.

In some embodiments, the targeting peptide comprises a sequence selected from SEQ ID NOs.: 238-337. In some embodiments, the targeting peptide comprises a sequence selected from SEQ ID NOs.: 238-437. In some embodiments, the targeting peptide comprises a sequence selected from SEQ ID NOs.: 238-537. In some embodiments, the targeting peptide comprises a sequence selected from SEQ ID NOs.: 238-637. In some embodiments, the targeting peptide comprises a sequence selected from SEQ ID NOs.: 238-737. In some embodiments, the targeting peptide comprises a sequence selected from SEQ ID NOs.: 238-837. In some embodiments, the targeting peptide comprises a sequence selected from SEQ ID NOs.: 238-937. In some embodiments, the targeting peptide comprises a sequence selected from SEQ ID NOs.: 238-1037.

In some embodiments, the targeting peptide is the targeting peptide disclosed in US2017/0166926, incorporated by reference in its entirety herein.

The targeting peptide can have any of the sequences selected from SEQ ID NOs: 2-51 and 53 provided herein. In some embodiments, the targeting peptide is the 7-mer peptide TLAVPFK (SEQ ID NO: 53).

In some embodiments, the targeting peptide is not a targeting peptide disclosed in US2017/0166926, WO2019/028306, WO2020/072683, WO2021/042909, WO2021/050974, WO2021/077000, or WO2021/222831, incorporated by reference in their entireties herein.

### 6.2.2. Targeting Peptide Site

A modified AAV capsid protein of the present disclosure comprises a targeting peptide within VR VIII of the reference AAV capsid protein (FIG. 1).

Preferably, the targeting peptide is at a site exposed to the exterior of the capsid, preferably based on structure predictions and/or experimental data. More preferably, the targeting peptide is at a site exposed to the exterior of the AAV capsid in a manner that does not interfere with the activity of said protein in capsid assembly.

The position of the targeting peptide in an AAV capsid protein that “corresponds to” the position in the AAV9 capsid protein can be established by the skilled person by known methods, preferably by aligning the amino acids of the capsid proteins. In some embodiments, the site of the targeting peptide corresponds to amino acid position 588 of the AAV9 VP1 capsid protein. In other words, X₁ of the targeting peptide is at a site corresponding to amino acid position 580 of the AAV9 VP1 capsid protein. In other words, X₁ of the targeting peptide is at a site corresponding to amino acid position 581 of the AAV9 VP1 capsid protein. In other words, X₁ of the targeting peptide is at a site corresponding to amino acid position 582 of the AAV9 VP1 capsid protein. In other words, X₁ of the targeting peptide is at a site corresponding to amino acid position 583 of the AAV9 VP1 capsid protein. In other words, X₁ of the targeting peptide is at a site corresponding to amino acid position 584 of the AAV9 VP1 capsid protein. In other words, X₁ of the targeting peptide is at a site corresponding to amino acid position 588 of the AAV9 VP1 capsid protein. In other words, X₁ of the targeting peptide is at a site corresponding to amino acid position 585 of the AAV9 VP1 capsid protein. In other words, X₁ of the targeting peptide is at a site corresponding to amino acid position 586 of the AAV9 VP1 capsid protein. In other words, X₁ of the targeting peptide is at a site corresponding to amino acid position 587 of the AAV9 VP1 capsid protein. In other words, X₁ of the targeting peptide is at a site corresponding to amino acid position 588 of the AAV9 VP1 capsid protein. In other words, X₁ of the targeting peptide is at a site corresponding to amino acid position 587 of the AAV9 VP1 capsid protein. In other words, X₁ of the targeting peptide is at a site corresponding to amino acid position 586 of the AAV9 VP1 capsid protein. In other words, X₁ of the targeting peptide is at a site corresponding to amino acid position 585 of the AAV9 VP1 capsid protein. In other words, X₁ of the targeting peptide is at a site corresponding to amino acid position 589 of the AAV9 VP1 capsid protein. In other words, X₁ of the targeting peptide is at a site corresponding to amino acid position 590 of the AAV9 VP1 capsid protein. In other words, X₁ of the targeting peptide is at a site corresponding to amino acid position 591 of the AAV9 VP1 capsid protein. In other words, X₁ of the targeting peptide is at a site corresponding to amino acid position 592 of the AAV9 VP1 capsid protein.

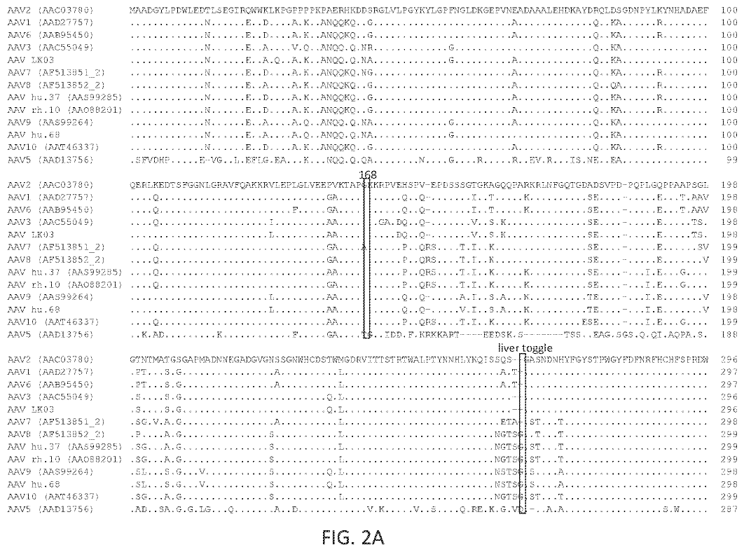

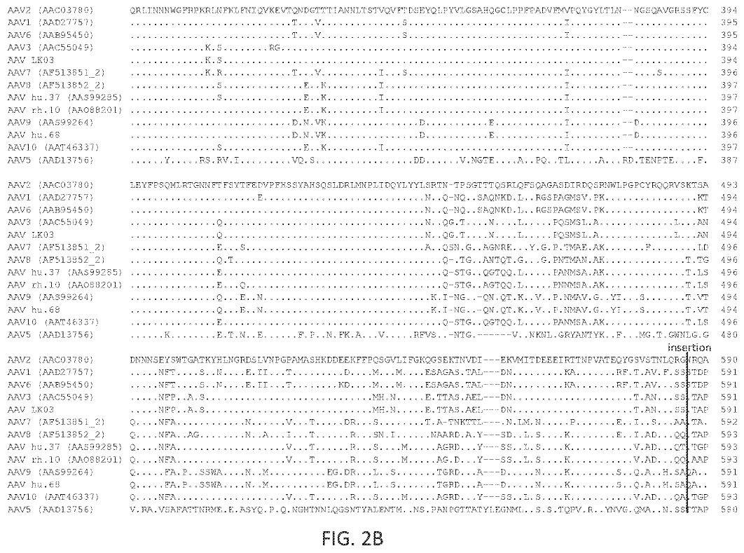

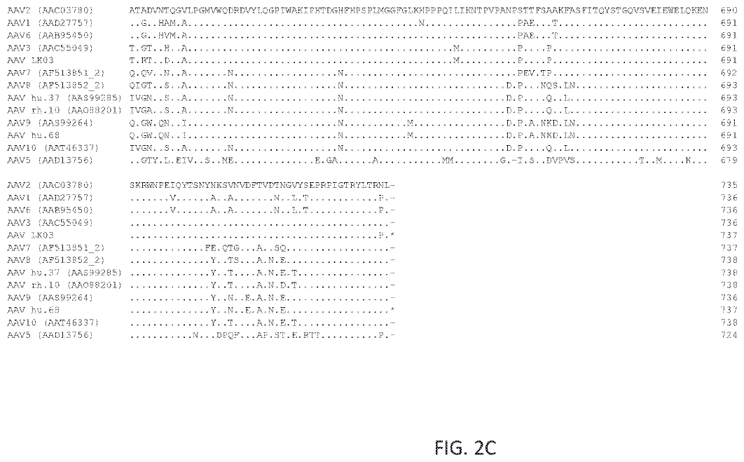

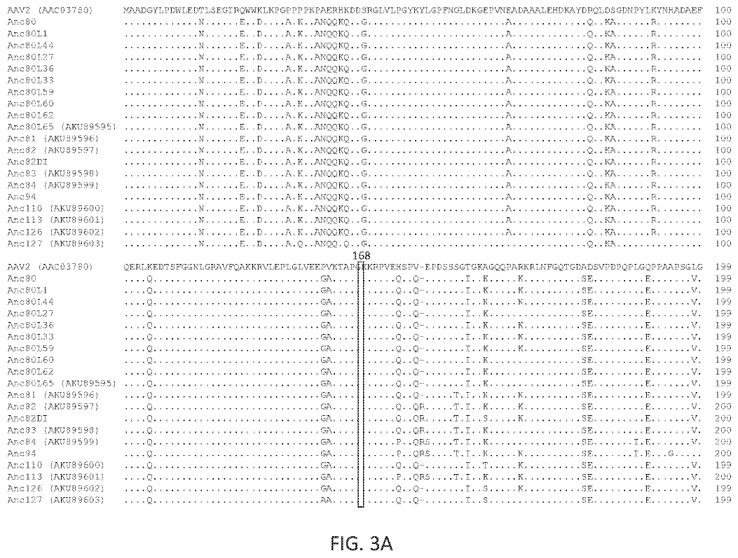

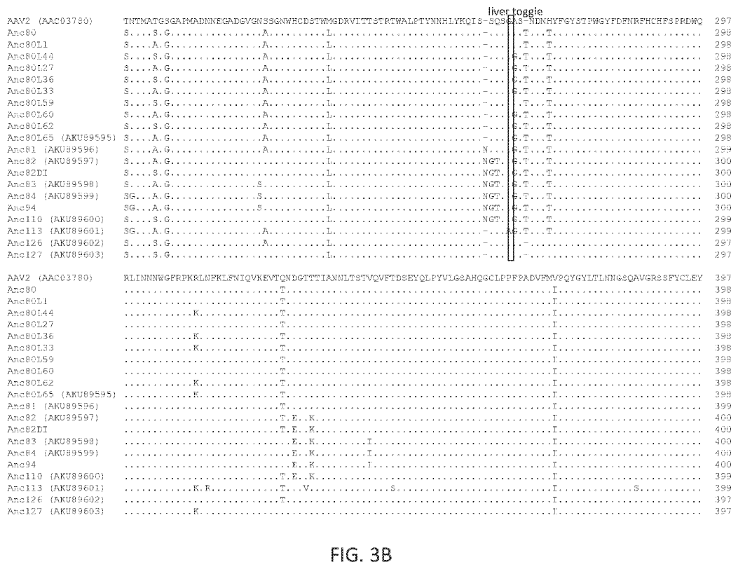

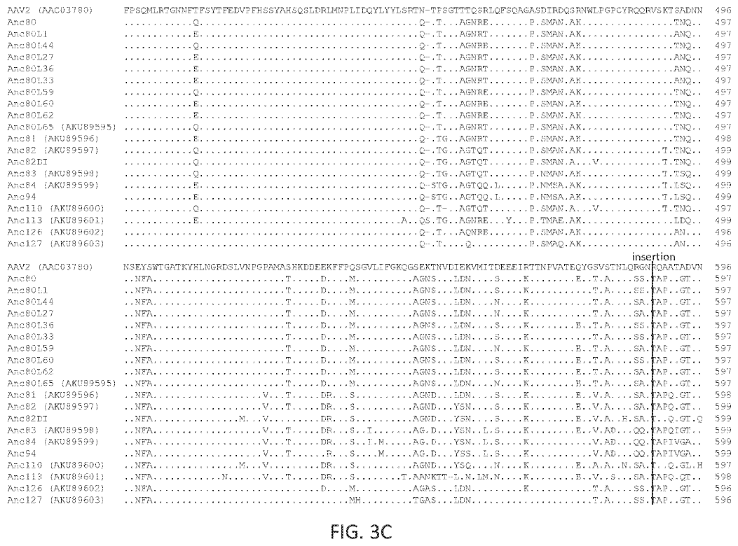

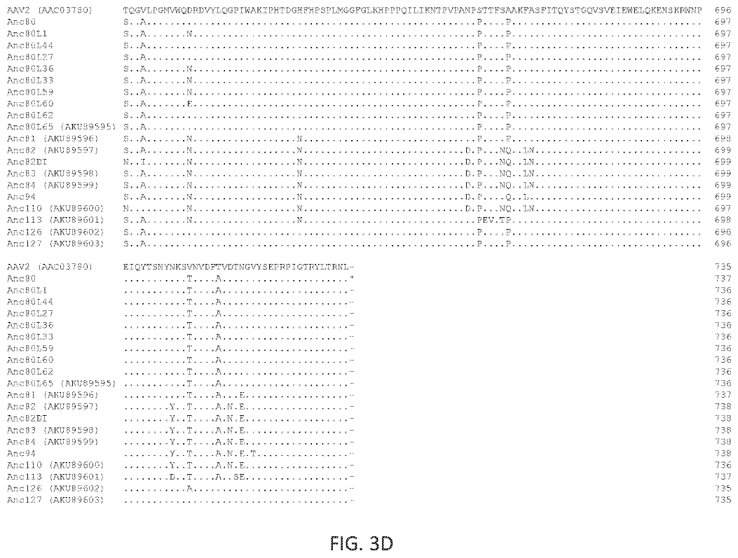

The position of the targeting peptide can be any one of those described in WO2019/207132, incorporated by reference in its entirety herein. Some of the sites are provided below in Table 1 and highlighted in FIGS. 2A-2C and FIGS. 3A-3D. In Table 1, the preferred sites are indicated by a “-” relative to wild type VP1 capsid polypeptide.

TABLE 1
Exemplary insertion sites

| | Insertion sites 1 | | Insertion sites 2 | | Insertion sites 3 | |
|---|---|---|---|---|---|---|
| AAV1 | D590 | STD- | S588 | SSS- | Q585 | Q-SSS-T |
| | P591 | PAT | T589 | TDP | T589 | |
| AAV2 | R588 | GNR- | N587 | RGN- | Q584 | Q-RGN-R |
| | Q589 | QAA | R588 | RQA | R588 | |
| AAV3b | S586 | LQS- | N588 | SSN- | Q585 | Q-SSN-T |
| | S587 | SNT | T589 | TAP | T589 | |
| AAV4 | S584 | DQS- | S586 | SNS- | G581 | G-DQS-N |
| | N585 | NSN | N587 | NLP | N585 | |
| AAV5 | S575 | NQS- | T577 | SST- | Q574 | Q-SST-T |
| | S576 | STT | T578 | TAP | T578 | |
| AAV6 | DS90 | STD- | S588 | SSS- | Q585 | Q-SSS-T |
| | P591 | PAT | T589 | TDP | T589 | |
| AAV7 | N589 | AAN- | | | Q586 | Q-AAN-T |
| | T590 | TAA | | | T590 | |
| AAV8 | N590 | QQN- | | | Q587 | Q-QQN-T |
| | T591 | TAP | | | T591 | |
| AAV9 | Q588 | SAQ- | | | Q585 | Q-SAQ-A |
| | A589 | AQA | | | A589 | |
| AAVrh10 | N590 | QQN- | | | Q587 | Q-QQN-A |
| | A591 | AAP | | | A591 | |
| AAVpo.1 | N567 | NQN- | N569 | NSN- | N564 | N-NQN-S |
| | S568 | SNT | T570 | THP | S568 | |
| AAV12 | N592 | NQN- | T594 | NAT- | N589 | N-NQN-A |
| | A593 | ATT | T595 | TAP | A593 | |
| Anc80 | N588 | QSXN-T | | | Q585 | Q-SXN-T |
| | T589 | | | | T589 | |
| Anc80L65 | N588 | QSAN-T | | | Q585 | Q-SAN-T |
| | T589 | | | | T589 | |
| Anc80-55 | N588 | QSSN-T | | | Q585 | Q-SSN-T |
| | T589 | | | | T589 | |
| Anc80-129 | N588 | QSAN-T | | | Q585 | Q-SAN-T |
| | T589 | | | | T589 | |
| Anc80-156 | N588 | QSAN-T | | | Q585 | Q-SAN-T |
| | T589 | | | | T589 | |
| Anc80-751 | N588 | QSAN-T | | | Q585 | Q-SAN-T |
| | T589 | | | | T589 | |
| Anc80-1029 | N588 | QSAN-T | | | Q585 | Q-SAN-T |
| | T589 | | | | T589 | |
| Anc80-1712 | N588 | QSAN-T | | | Q585 | Q-SAN-T |
| | T589 | | | | T589 | |

3 exemplary insertion sites are indicated in Table 1. For insertion sites 1 and 2, the targeting peptide is inserted between 2 amino acid positions, where the insertion site is denoted as “-”. For example, for AAV1 capsid protein insertion site 1, the targeting peptide is inserted between positions D590 and P591. In some embodiments, the AAV capsid protein is modified by way of mutation or substitution of one or more amino acids, followed by insertion of the targeting peptide. For example, for insertion site 3 of Table 1, 3 amino acids positioned between the insertion sites are substituted or mutated prior to insertion of the targeting peptide. For example, for AAV1 capsid protein insertion site 3, the targeting peptide is inserted between position Q585 and T589 after the amino acids “SSS” positioned between Q585 and T589 (S586, S587, and S588) are deleted, denoted as “Q-SSS-T”.

In some embodiments, the targeting peptide is at between 560 and 600 within the VR VIII of the modified AAV capsid protein. In some embodiments, the targeting peptide is at between 565 and 595 within the VR VIII of the modified AAV capsid protein.

In some embodiments, the targeting peptide is at between 570 and 610 within the VR VIII of the modified AAV capsid protein. In some embodiments, the targeting peptide is at between 580 and 610 within the VR VIII of the modified AAV capsid protein. In some embodiments, the targeting peptide is at between 580 and 595 within the VR VIII of the modified AAV capsid protein. In some embodiments, the targeting peptide is at between 582 and 600 within the VR VIII of the modified AAV capsid protein.

In some embodiments, the reference AAV capsid protein is a capsid protein of AAV1 or a modification thereof and the targeting peptide is between D590 and P591 or between S588 and T589 of the modified AAV capsid protein. In some embodiments, the reference AAV capsid protein is a capsid protein of AAV1 or a modification thereof and the targeting peptide is between positions 587 and 594 or between positions 585 and 592 of the modified AAV capsid protein. In some embodiments, the reference AAV capsid protein is a capsid protein of AAV1 or a modification thereof and the targeting peptide is between Q585 and TS89 of the reference AAV capsid protein.

In some embodiments, the reference AAV capsid protein is a capsid protein of AAV2 or a modification thereof and the targeting peptide is between R585 and Q589 or between N587 and R588 of the modified AAV capsid protein. In some embodiments, the reference AAV capsid protein is a capsid protein of AAV2 or a modification thereof and the targeting peptide is between positions 582 and 592 or between positions 585 and 591 of the modified AAV capsid protein. In some embodiments, the reference AAV capsid protein is a capsid protein of AAV2 or a modification thereof and the targeting peptide is between Q584 and R588.

In some embodiments, the reference AAV capsid protein is a capsid protein of AAV3 or a modification thereof and the targeting peptide is between S586 and S587 or between N588 and T589 of the modified AAV capsid protein. In some embodiments, the reference AAV capsid protein is a capsid protein of AAV3 or a modification thereof and the targeting peptide is between positions 583 and 590 or between positions 585 and 592 of the modified AAV capsid protein. In some embodiments, the reference AAV capsid protein is a capsid protein of AAV3 or a modification thereof and the targeting peptide is between Q585 and T589 of the reference AAV capsid protein.

In some embodiments, the reference AAV capsid protein is a capsid protein of AAV4 and the targeting peptide is between S584 and N585 or between S586 and N587 of the modified AAV capsid protein. In some embodiments, the reference AAV capsid protein is a capsid protein of AAV4 or a modification thereof and the targeting peptide is between positions 581 and 586 or between positions 583 and 590 of the modified AAV capsid protein. In some embodiments, the reference AAV capsid protein is a capsid protein of AAV4 or a modification thereof and the targeting peptide is between G581 and N585 of the reference AAV capsid protein.

In some embodiments, the reference AAV capsid protein is a capsid protein of AAV5 or a modification thereof and the targeting peptide is between S575 and S576 or between T577 and T578 of the capsid protein. In some embodiments, the reference AAV capsid protein is a capsid protein of AAV5 or a modification thereof and the targeting peptide is between positions 572 and 579 or between positions 574 and 581 of the capsid protein. In some embodiments, the reference AAV capsid protein is a capsid protein of AAV5 and the targeting peptide is between Q574 and T589 of the reference AAV capsid protein.

In some embodiments, the reference AAV capsid protein is a capsid protein of AAV6 or a modification thereof and the targeting peptide is between D590 and P591 or S588 and T589 of the modified AAV capsid protein. In some embodiments, the reference AAV capsid protein is a capsid protein of AAV6 or a modification thereof and the targeting peptide is between positions 587 and 594 or positions 585 and 592 of the modified AAV capsid protein. In some embodiments, the reference AAV capsid protein is a capsid protein of AAV6 or a modification thereof and the targeting peptide is between Q585 and T589 of the reference AAV capsid protein.

In some embodiments, the reference AAV capsid protein is a capsid protein of AAV7 or a modification thereof and the targeting peptide is between N589 and T590 of the modified AAV capsid protein. In some embodiments, the reference AAV capsid protein is a capsid protein of AAV7 or a modification thereof and the targeting peptide is between positions 586 and 593 of the modified AAV capsid protein. In some embodiments, the reference AAV capsid protein is a capsid protein of AAV7 or a modification thereof and the targeting peptide is between Q586 and T590 of the reference AAV capsid protein.

In some embodiments, the reference AAV capsid protein is a capsid protein of AAV8 and the targeting peptide is between N590 and T591 of the modified AAV capsid protein. In some embodiments, the reference AAV capsid protein is a capsid protein of AAV8 or a modification thereof and the targeting peptide is between positions 587 and 594 of the modified AAV capsid protein. In some embodiments, the reference AAV capsid protein is a capsid protein of AAV8 or a modification thereof and the targeting peptide is between Q587 and T591 of the modified AAV capsid protein.

In some embodiments, the reference AAV capsid protein is a capsid protein of AAV9 and the targeting peptide is between Q588 and A589 of the modified AAV capsid protein. In some embodiments, the reference AAV capsid protein is a capsid protein of AAV9 or a modification thereof and the targeting peptide is between positions 585 and 592 of the modified AAV capsid protein. In some embodiments, the reference AAV capsid protein is a capsid protein of AAV9 or a modification thereof and the targeting peptide is between Q585 and A589 of the reference AAV capsid protein.

In some embodiments, the reference AAV capsid protein is a capsid protein of AAVrh10 or a modification thereof and the targeting peptide is between N590 and A591 of the modified AAV capsid protein. In some embodiments, the reference AAV capsid protein is a capsid protein of AAVrh10 or a modification thereof and the targeting peptide is between positions 587 and 594 of the modified AAV capsid protein. In some embodiments, the reference AAV capsid protein is a capsid protein of AAVrh10 or a modification thereof and the targeting peptide is between Q587 and A591 of the reference AAV capsid protein.

In some embodiments, the reference AAV capsid protein is a capsid protein of AAVpo.1 or a modification thereof and the targeting peptide is between N567 and S568 or between N569 and T570 of the modified AAV capsid protein. In some embodiments, the reference AAV capsid protein is a capsid protein of AAVpo.1 or a modification thereof and the targeting peptide is between positions 570 and 571 or between positions 566 and 573 of the modified AAV capsid protein. In some embodiments, the reference AAV capsid protein is a capsid protein of AAVpo.1 or a modification thereof and the targeting peptide is between N564 and S568 of the reference AAV capsid protein.

In some embodiments, the reference AAV capsid protein is a capsid protein of AAV12 or a modification thereof and the targeting peptide is between N592 and A593 or between T594 and T595 of the modified AAV capsid protein. In some embodiments, the reference AAV capsid protein is a capsid protein of AAV12 or a modification thereof and the targeting peptide is between positions 589 and 596 or between positions 591 and 598 of the modified AAV capsid protein. In some embodiments, the reference AAV capsid protein is a capsid protein of AAV12 or a modification thereof and the targeting peptide is between N589 and A593 of the reference AAV capsid protein.

In some embodiments, the reference AAV capsid protein is a capsid protein of Anc80 or a modification thereof and the targeting peptide is between T589 and A590 or between N587 and T588 of the modified AAV capsid protein. In some embodiments, the reference AAV capsid protein is a capsid protein of Anc80 or a modification thereof and the targeting peptide is between positions 586 and 593 or between positions 584 and 591 of the modified AAV capsid protein. In some embodiments, the reference AAV capsid protein is a capsid protein of Anc80 or a modification thereof and the targeting peptide is between Q585 and T589 of the reference AAV capsid protein.

In some embodiments, the reference AAV capsid protein is a capsid protein of Anc80L65 or a modification thereof and the targeting peptide is between T589 and A590 or between N587 and T588 of the modified AAV capsid protein. In some embodiments, the reference AAV capsid protein is a capsid protein of Ane80L65 or a modification thereof and the targeting peptide is between positions 586 and 593 or between positions 584 and 591 of the modified AAV capsid protein. In some embodiments, the reference AAV capsid protein is a capsid protein of Anc80L65 or a modification thereof and the targeting peptide is between Q585 and T589 of the reference AAV capsid protein.

In some embodiments, the reference AAV capsid protein is a capsid protein of Anc80-55 or a modification thereof and the targeting peptide is between T589 and A590 or between N587 and T588 of the modified AAV capsid protein. In some embodiments, the reference AAV capsid protein is a capsid protein of Anc80-55 or a modification thereof and the targeting peptide is between positions 586 and 593 or between positions 584 and 591 of the modified AAV capsid protein. In some embodiments, the reference AAV capsid protein is a capsid protein of Anc80-55 or a modification thereof and the targeting peptide is between Q585 and T589 of the reference AAV capsid protein.

In some embodiments, the reference AAV capsid protein is a capsid protein of Anc80-129 or a modification thereof and the targeting peptide is between T589 and A590 or between N587 and T588 of the modified AAV capsid protein. In some embodiments, the reference AAV capsid protein is a capsid protein of Anc80-129 or a modification thereof and the targeting peptide is between positions 586 and 593 or between positions 584 and 591 of the modified AAV capsid protein. In some embodiments, the reference AAV capsid protein is a capsid protein of Anc80-129 or a modification thereof and the targeting peptide is between Q585 and T589 of the reference AAV capsid protein.

In some embodiments, the reference AAV capsid protein is a capsid protein of Anc80-156 or a modification thereof and the targeting peptide is between T589 and A590 or between N587 and T588 of the modified AAV capsid protein. In some embodiments, the reference AAV capsid protein is a capsid protein of Anc80-156 or a modification thereof and the targeting peptide is between positions 586 and 593 or between positions 584 and 591 of the modified AAV capsid protein. In some embodiments, the reference AAV capsid protein is a capsid protein of Anc80-156 or a modification thereof and the targeting peptide is between Q585 and T589 of the reference AAV capsid protein.

In some embodiments, the reference AAV capsid protein is a capsid protein of Anc80-751 or a modification thereof and the targeting peptide is between T589 and A590 or between N587 and T588 of the modified AAV capsid protein. In some embodiments, the reference AAV capsid protein is a capsid protein of Anc80-751 or a modification thereof and the targeting peptide is between positions 586 and 593 or between positions 584 and 591 of the modified AAV capsid protein. In some embodiments, the reference AAV capsid protein is a capsid protein of Anc80-751 or a modification thereof and the targeting peptide is between Q585 and T589 of the reference AAV capsid protein.

In some embodiments, the reference AAV capsid protein is a capsid protein of Anc80-1029 or a modification thereof and the targeting peptide is between T589 and A590 or between N587 and T588 of the modified AAV capsid protein. In some embodiments, the reference AAV capsid protein is a capsid protein of Anc80-1029 or a modification thereof and the targeting peptide is between positions 586 and 593 or between positions 584 and 591 of the modified AAV capsid protein. In some embodiments, the reference AAV capsid protein is a capsid protein of Anc80-1029 or a modification thereof and the targeting peptide is between Q585 and T589 of the reference AAV capsid protein.

In some embodiments, the reference AAV capsid protein is a capsid protein of Anc80-1712 or a modification thereof and the targeting peptide is between T589 and A590 or between N587 and T588 of the modified AAV capsid protein. In some embodiments, the reference AAV capsid protein is a capsid protein of Anc80-1712 or a modification thereof and the targeting peptide is between positions 586 and 593 or between positions 584 and 591 of the modified AAV capsid protein. In some embodiments, the reference AAV capsid protein is a capsid protein of Anc80-1712 or a modification thereof and the targeting peptide is between Q585 and T589 of the reference AAV capsid protein.

### 6.2.3. Various Embodiments of Modified AAV Capsid Proteins

The present disclosure provides a modified AAV capsid protein comprising a reference AAV capsid protein with one or more modifications to comprise a targeting peptide at a site within VR VIII of the reference AAV capsid protein, wherein the targeting peptide has a sequence X₁X₂X₃RGDX₇X₈X₉X₁₀. In some embodiments, the modified AAV capsid protein comprises a targeting peptide selected from SEQ ID NOs.: 238-44858 introduced into the VR VIII. In some embodiments, the modified AAV capsid protein comprises a targeting peptide having at least 90%, at least 95%, at least 98%, at least 99% or at least 100% sequence identity to the amino acid sequence selected from SEQ ID NOs. 238-44858 introduced into the VRVIII.

In some embodiments, the modified AAV capsid protein is an AAV9 capsid protein containing a targeting peptide, RGDLLLS (SEQ ID NO: 1), inserted into the VR VIII. In one embodiment, the modified AAV capsid protein has a sequence of SEQ ID NO: 158. In some embodiments, the modified AAV capsid protein has the amino acids 138 to 736 of SEQ ID NO: 158. In some embodiments, the modified AAV capsid protein has the amino acids 203 to 736 of SEQ ID NO: 158. In some embodiments, the modified AAV capsid protein has the amino acid sequence selected from SEQ ID NOs.: 44900-44909. In some embodiments, the modified AAV capsid protein has the amino acid sequence of SEQ ID NO.: 44900. In some embodiments, the modified AAV capsid protein has the amino acid sequence of SEQ ID NO: 44901. In some embodiments, the modified AAV capsid protein has the amino acid sequence of SEQ ID NO.: 44902. In some embodiments, the modified AAV capsid protein has the amino acid sequence of SEQ ID NO.: 44903. In some embodiments, the modified AAV capsid protein has the amino acid sequence of SEQ ID NO.: 44904 In some embodiments, the modified AAV capsid protein has the amino acid sequence of SEQ ID NO.: 44905. In some embodiments, the modified AAV capsid protein has the amino acid sequence of SEQ ID NO.: 44906. In some embodiments, the modified AAV capsid protein has the amino acid sequence of SEQ ID NO.: 44907. In some embodiments, the modified AAV capsid protein has the amino acid sequence of SEQ ID NO.: 44908. In some embodiments, the modified AAV capsid protein has the amino acid sequence of SEQ ID NO.: 44909. some embodiments, the modified AAV capsid protein has a sequence having at least 90%, 91%, 92%, 93%, 94%, 95%, 96%, 97%, 98%, or 99% identity to an amino acid sequence selected from: SEQ ID NOs.: 44900-44909.

In some embodiments, the modified AAV capsid protein has a sequence having at least 90%, 91%, 92%, 93%, 94%, 95%, 96%, 97%, 98%, or 99% identity to SEQ ID NO: 158.

In another aspect, the present disclosure provides a modified AAV capsid protein comprising (i) a) an alanine (A) amino acid residue at an amino acid position corresponding to position 266 in Anc80; or b) a lysine (K) amino acid residue at an amino acid position corresponding to position 168 in Anc80; and (it) a targeting peptide introduced into a site within VR VIII of the liver-toggle mutant.

In one embodiment, the modified AAV capsid protein is an AAV9 capsid protein containing a targeting peptide, RGDLLLS (SEQ ID NO: 1), inserted into the VR VIII. The modified AAV capsid protein can comprise one or more additional modifications to comprise (i) a) an alanine (A) amino acid residue at an amino acid position corresponding to position 266 in Anc80; or b) a lysine (K) amino acid residue at an amino acid position corresponding to position 168 in Anc80. In one embodiment, the modified AAV capsid protein has a sequence of SEQ ID NO: 159. In some embodiments, the modified AAV capsid protein has the amino acids 138 to 736 of SEQ ID NO: 159. In some embodiments, the modified AAV capsid protein has the amino acids 203 to 736 of SEQ ID NO: 159.

In some embodiments, the modified AAV capsid protein has a sequence having at least 90%, 91%, 92%, 93%, 94%, 95%, 96%, 97%, 98%, or 99% identity to SEQ ID NO: 159.

A modified AAV capsid protein of the present disclosure can change the tropism, specificity and/or bio-distribution of an AAV comprising the modified AAV capsid protein. In preferred embodiments, an AAV comprising the modified AAV capsid protein has increased targeting to a target cell, tissue or organ when administered to a subject. In some embodiments, an AAV comprising the modified AAV capsid protein has decreased distribution outside of a target cell, tissue or organ when administered to a subject. In some embodiments, tropism of a modified AAV capsid protein can be measured using an enrichment score. A non-limiting example of an enrichment score is based on the combination of amino acid residues present in the modified sequence within VR VIII. An exemplary enrichment formula is provided below:

$$Tissue Enrichment score = log 2 FoldChange = log 2 {MN}_{Tissue} - log 2 {MN}_{TestArticle}$$

The formula for the scaled log 2 fold change is provided below:

$$Scaled log 2 FoldChange = ( log 2 FoldChange - min ( log 2 FoldChange ) ) / ( max ( log 2 FoldChange ) - min ( log 2 FC ) )$$

In some embodiments, the modified AAV capsid protein comprises (i) a reference AAV capsid protein, and (ii) a 7-mer peptide having the sequence RGDLLLS (SEQ ID NO: 1) inserted into a site within VR VIII of the reference AAV capsid protein.

### 6.2.4. Reference AAV Capsid Proteins

The reference AAV capsid protein used in various embodiments of the present disclosure is a VP1, VP2 or VP3 capsid protein of an AAV known in the art. It can be a VP1, VP2 or VP3 capsid protein of a naturally occurring or non-naturally occurring AAV variant.

The non-naturally occurring VP1, VP2, or VP3 capsid protein includes a capsid protein generated by biological or chemical alteration or in silico design, or variation of a naturally occurring AAV capsid protein. Accordingly, the reference AAV capsid protein includes, but is not limited to, a capsid protein of various AAV serotypes (e.g., AAV1, AAV2, AAV3B, AAV5, AAV6, AAV8, and AAV9) or a variant thereof. A non-naturally occurring VP1, VP2, or VP3 capsid protein further includes an artificial capsid protein created by in silico design or synthesis. An artificial capsid protein includes, but is not limited to, AAV capsid proteins disclosed in PCT/US2014/060163, U.S. Pat. No. 9,695,220, PCT/US2016/044819, PCT/US2018/032166, PCT/US2019/031851, and PCT/US2019/047546, which are incorporated herein by reference in their entireties.

In some embodiments, the reference AAV capsid protein is the capsid protein of AAV9 (Genbank Acc. No: AAS99264.1), AAV1 (Genbank Acc. No: AAD27757.1), AAV2 (Genbank Acc. No: AAC03780.1). AAV3 (Genbank Acc. No: AAC55049.1), AAV3b (Genbank Acc. No: AF028705.1), AAV4 (Genbank Acc. No: AAC58045.1), AAV5 (Genbank Acc. No: AAD13756.1), AAV6 (Genbank Acc. No: AF028704.1), AAV7 (Genbank Acc. No: AAN03855.1), AAV 8 (Genbank Acc. No: AAN03857.1), AAV10 (Genbank Acc. No: AAT46337.1), AAVrh10 (Genbank Acc. No: AY243015.1), AAV1I (Genbank Acc. No: AAT46339.1), AAV12 (Genbank Acc. No: ABI16639.1), or AAV13 (Genbank Acc. No: ABZ10812.1), AAVpol (Genbank Acc. No: FJ688147.1). In certain embodiments, the AAV capsid protein is the capsid protein of AAV9 (Genbank Acc. No: AAS99264.1).

The reference AAV capsid protein can be VP1 capsid protein having a sequence selected from: SEQ ID NO: 54 (AAV1 (AAD27757)), SEQ ID NO: 55 (AAV2 (AAC03780), SEQ ID NO: 56 (AAV3 (AAC55049)), SEQ ID NO: 57 (AAV5 (AAD13756)), SEQ ID NO: 58 (AAV6 (AAB95450), SEQ ID NO: 59 (AAV7 (AF513851_2), SEQ ID NO: 60 (AAV8 (AF513852_2)), SEQ ID NO: 61 (AAV9 (AAS99264), SEQ ID NO: 62 (AAV10 (AAT46337)), SEQ ID NO: 63 (AAV hu.68), SEQ ID NO: 64 (AAV LK03), SEQ ID NO: 65 (AAV hu.1 (AAS99260)), SEQ ID NO: 66 (AAV hu.2 (AAS99270)), SEQ ID NO: 67 (AAV hu.3 (AAS99280), SEQ ID NO: 68 (AAV hu.4 (AAS99287), SEQ ID NO: 69 (AAV hu.6 (AAS99306), SEQ ID NO: 70 (AAV hu.7 (AAS99313)), SEQ ID NO: 71 (AAV hu.9 (AAS99314)), SEQ ID NO: 72 (AAV hu.10 (AAS99261)), SEQ ID NO: 73 (AAV hu.11 (AAS99262)), SEQ ID NO: 74 (AAV hu.15 (AAS99265)), SEQ ID NO: 75 (AAV hu.16 (AAS99266)), SEQ ID NO: 76 (AAV hu.17 (AAS99267), SEQ ID NO: 77 (AAV hu.18 (AAS99268)), SEQ ID NO: 78 (AAV hu.20 (AAS99271)), SEQ ID NO: 79 (AAV hu.21 (AAS99272)), SEQ ID NO: 80 (AAV hu.22 (AAS99273), SEQ ID NO: 81 (AAV hu.23 (AAS99274)), SEQ ID NO: 82 (AA V hu.25 (AAS99276)), SEQ ID NO: 83 (AAV hu.27 (AAS99277)), SEQ ID NO: 84 (AAV hu.28 (AAS99278)), SEQ ID NO: 85 (AAV hu.29 (AAS99279)), SEQ ID NO. 86 (AAV hu.31 (AAS99281)), SEQ ID NO: 87 (AAV hu.32 (AAS99282)), SEQ ID NO: 88 (AAV hu.34 (AAS99283)), SEQ ID NO: 89 (AAV hu.37 (AAS99285), SEQ ID NO: 90 (AAV hu.39 (AAS99286)), SEQ ID NO: 91 (AAV hu.41 (AAS99289), SEQ ID NO: 92 (AAV hu.42 (AAS99290), SEQ ID NO: 93 (AAV hu.43 (AAS99291)), SEQ ID NO: 94 (AAV hu.44 (AAS99292), SEQ ID NO: 95 (AAV hu.45 (AAS99293)), SEQ ID NO: 96 (AAV hu.46 (AAS99294)), SEQ ID NO: 97 (AAV hu.47 (AAS99295)), SEQ ID NO: 98 (AAV hu.48 (AAS99296)), SEQ ID NO: 99 (AAV hu.51 (AAS99298)), SEQ ID NO: 100 (AAV hu.52 (AAS99299)), SEQ ID NO: 101 (AAV hu.53 (AAS99300)), SEQ ID NO: 102 (AAV hu.54 (AAS99301)), SEQ ID NO: 103 (AAV hu.55 (AAS99302)), SEQ ID NO: 104 (AAV hu.56 (AAS99303)), SEQ ID NO: 105 (AAV hu.57 (AAS99304), SEQ ID NO: 106 (AAV hu.60 (AAS99307)), SEQ ID NO: 107 (AAV hu.61 (AAS99308), SEQ ID NO. 108 (AAV hu.63 (AAS99309), SEQ ID NO: 109 (AAV hu.66 (AAS99311)), SEQ ID NO: 110 (AAV hu.67 (AAS99312)), SEQ ID NO: 111 (AAV rh.10 (AAO88201)), SEQ ID NO: 112 (AAV rh.13 (AAO88199)), SEQ ID NO: 113 (AAV rh.19 (AAO88194)), SEQ ID NO: 114 (AAV rh.22 (AAO88192)), SEQ ID NO: 115 (AAV rh.23 (AAO88191)), SEQ ID NO: 116 (AAV rh.24 (AAO88190), SEQ ID NO: 117 (AAV rh.35 (AAO88186), SEQ ID NO: 118 (AAV rh.43 (AAS99245), SEQ ID NO: 119 (AAV rh.48 (AAS99246), SEQ ID NO: 120 (AAV rh.49 (AAS99247), SEQ ID NO: 121 (AAV rh.50 (AAS99248), SEQ ID NO. 122 (AAV rh.51 (AAS99249), SEQ ID NO: 123 (AAV rh.52 (AAS99250)), SEQ ID NO: 124 (AAV rh.53 (AAS99251)), SEQ ID NO: 125 (AAV rh.54 (AAS99252)), SEQ ID NO: 126 (AAV rh.55 (AAS99253)), SEQ ID NO: 127 (AAV rh.57 (AAS99254)), SEQ ID NO: 128 (AAV rh.58 (AAS99255)), SEQ ID NO: 129 (AAV rh.62 (AAS99258)), SEQ ID NO: 130 (AAV rh.64 (AAS99259)), SEQ ID NO: 131 (AAV rh.56 (JA400164)), SEQ ID NO: 143 (Anc80L1), SEQ ID NO: 144 (Anc80L27), SEQ ID NO: 145 (Anc80L33), SEQ ID NO: 146 (Anc80L36), SEQ ID NO: 147 (Anc80L44), SEQ ID NO: 148 (Anc80L59), SEQ ID NO: 149 (Anc80L60), SEQ ID NO: 150 (Anc80L62), SEQ ID NO: 151 (Anc82DI), SEQ ID NO: 152 (AAV rh.74). Anc80-55 (SEQ ID NO: 44885); Anc80-129 (SEQ ID NO: 44887); Anc80-156 (SEQ ID NO: 44889); Anc80-751 (SEQ ID NO: 44916); Anc80-1029 (SEQ ID NO: 44917); and Anc80-1712 (SEQ ID NO: 44893). The reference AAV capsid protein can be a VP2 or VP3 protein having a part of one of the sequences. For example, VP2 protein can have a sequence corresponding to amino acids 138 to 736 of AAV9 VP1 and VP3 protein can have a sequence corresponding to amino acids 138 to 736 of AAV9 VP1 protein.

The reference AAV capsid protein can be VP1 capsid protein having any member sequence of the ancestral AAV library selected from SEQ ID NO: 132 (Anc80), SEQ ID NO: 133 (Anc8) (AKU89596)), SEQ ID NO: 134 (Anc82 (AKU89597)), SEQ ID NO: 135 (Anc83 (AKU89598), SEQ ID NO: 136 (Anc84 (AKU89599)), SEQ ID NO: 137 (Anc94) SEQ ID NO: 138 (Anc110 (AKU89600)), SEQ ID NO: 139 (Anc113 (AKU89601)), SEQ ID NO: 140 (Anc126 (AKU89602)), SEQ ID NO: 141 Anc127 (AKU89603), and SEQ ID NO: 142 (Anc80L65 (AKU89595)). The reference AAV capsid protein can be a VP2 or VP3 protein having a part of one of the sequences. For example, VP2 protein can have a sequence corresponding to amino acids 138 to 736 of AAV9 VP1 and VP3 protein can have a sequence corresponding to amino acids 138 to 736 of AAV9 VP1 protein. When a SEQ ID NO for a library sequence is used in this disclosure, it refers to a sequence of any one member of the library.

In some embodiments, the reference AAV capsid protein is a liver-toggle mutant described in WO2019/217911, which is incorporated by reference in its entirety herein.

In some embodiments, the reference AAV capsid protein is a capsid protein (VP1, VP2 or VP3) of an AAV variant selected from the group consisting of: AAV2; AAV1; AAV6; AAV3; AAV LK03; AAV7; AAV8; AAV hu.37; AAV rh.10; AAV9; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B, rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu.9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; hu.42-E; rh.57-E; rh.40-E; rh74; hu.67-E; hu.17-E; hu.6-E; hu.66-E; rh.38-E; hu.32-F; AAV9/hu; hu.31-F; Anc80L.27; Anc80159; Anc80L60; Anc80L62; Anc80L65; Anc80L33; Anc80L36; Anc80L44; Anc80L1; Anc80-55, Anc80-129, Anc80-156, Anc80-751, Anc80-1029, Anc80-1712; Anc110; and Anc80DI. In some embodiments, the reference AAV capsid protein is a capsid protein of any member protein of an ancestral AAV library selected from: Anc80; Anc81; Anc82; Ane83; Anc84; Anc94; Anc113; Anc126; and Anc127.

In some embodiments, the reference AAV capsid protein is a protein having a sequence selected from SEQ ID Nos: 54-131 and 143-152. In some embodiments, the reference AAV capsid protein is a protein having a VP2 (corresponding to amino acids 138 to 736 of AAV9 VP1) or VP3 portion (corresponding to amino acids 138 to 736 of AAV9 VP1) of the protein having a sequence selected from SEQ ID NOs: 54-131 and 143-152.

In some embodiments, the reference AAV capsid protein is a capsid protein of the AAV variant modified to include one or more liver-toggle mutations described in WO2019/217911. In some embodiments, the reference AAV capsid protein comprises (1) an alanine (A) or glycine (G) amino acid residue at an amino acid position corresponding to position 266 in Anc80 VP1 and/or (2) a lysine (K) or arginine (R) amino acid residue at an amino acid position corresponding to position 168 in Anc80 VP1. In some embodiments, the reference AAV capsid protein comprises (i) an alanine (A) amino acid residue at an amino acid position corresponding to position 266 in Anc80 VP1 and/or b) a lysine (K) amino acid residue at an amino acid position corresponding to position 168 in Anc80 VP1. In some embodiments, the reference AAV capsid protein comprises (ii) an alanine (A) amino acid residue at an amino acid position corresponding to position 267 in AAV9 VP1 protein and/or a threonine (T) amino acid residue at an amino acid position corresponding to position 269 in AAV9 VP1.

In some embodiments, the reference AAV capsid protein is a capsid protein having a sequence selected from SEQ ID NOs.: 44885-44898, 44916-44917, or a fragment thereof. In some embodiments, the reference AAV capsid protein is a capsid protein having a sequence with at least 90%, at least 95%, at least 98%, at least 99%, or at least 100% sequence identity to the amino acid sequence selected from SEQ ID NOs.: 44885-44898, 44916-44917, or a fragment thereof.

In some embodiments, the reference AAV capsid protein comprises one or more modifications as described in WO2019/217911 or WO 2021/050614, which are both incorporated by reference herein in their entireties. In some embodiments, the reference AAV capsid protein comprises one or more modifications as described in PCT Application No. PCT/US2022/015842, which is herein incorporated by reference in its entirety.

### 6.2.5. Peptide Segment

In some embodiments, the modified AAV capsid protein comprises a peptide segment within variable region I (VR I). In some embodiments, the modified AAV capsid protein comprises a tropism-altering peptide segment within VR I. In some embodiments, the modified AAV capsid protein comprises a peptide segment of 12 amino acids (P₁P₂P₃P₄P₅P₆P₇P₈P₉P₁₀P₁₁P₁₂) within VR I which is different from the reference AAV capsid protein. In some embodiments, among the 12 amino acids, only one amino acid is different from the amino acids in the corresponding positions within the reference AAV capsid protein. In some embodiments, more than one amino acid residues in the peptide segment are different from the amino acid residues in the corresponding positions within the reference AAV capsid protein.

In some embodiments, the peptide segment comprises 12 amino acids positioned between about amino acid 259 to about amino acid 275 in a reference AAV capsid protein (e.g., any of the reference AAV capsid proteins described herein). In some embodiments, the peptide segment comprises 12 amino acids (P₁P₂P₃P₄P₅P₆P₇P₈P₉P₁₀P₁₁P₁₂) located between position 261 and position 274 in a reference AAV capsid protein or between position 260 and position 273 in a reference AAV capsid protein. In some embodiments, the peptide segment comprises 11 amino acids (P₁P₂P₃P₄P₅P₆P₇P₈P₉P₁₀P₁₁P₁₂) between about amino acid 259 to about amino acid 275 in a reference AAV capsid protein (e.g., any of the reference AAV capsid proteins described herein). In some embodiments, the peptide segment comprises 10 amino acids between about amino acid 259 to about amino acid 275 in a reference AAV capsid protein (e.g., any of the reference AAV capsid proteins described herein).

In some embodiments, one or more amino acids in P₁P₂P₃P₄P₅P₆P₇P₈P₉P₁₀P₁₁P₁₂ are same as the one or more amino acids at corresponding positions within the reference AAV capsid protein.

In some embodiments, the peptide segment has a sequence selected from SEQ ID NOs: 44935-47387. In some embodiments, the peptide segment has a sequence having at least 80% or at least 90% sequence identity to a sequence selected from SEQ ID NOs: 44935-47387.

In some embodiments, a modified adeno-associated virus (AAV) capsid protein includes a reference AAV capsid protein with one or more modifications to comprise a peptide segment within variable region I (VR I) of the reference AAV capsid protein, wherein the peptide segment has an amino acid sequence of P₁P₂P₃P₄P₅P₆P₇P₈NDNP₁₂ and P₁, P₂, P₃, P₄, P₅, P₆, P₇, P₈, and P₁₂ are independently selected from any amino acid residue.

In some embodiments, a modified adeno-associated virus (AAV) capsid protein includes a peptide segment within variable region I (VR I), wherein the peptide segment has an amino acid sequence of P₁P₂P₃P₄P₅P₆P₇P₈NDNP₁₂ and P₁, P₂, P₃, P₄, P₅, P₆, P₇, P₈, and P₁₂ are independently selected from any amino acid residue.

In some embodiments, the modified AAV capsid protein can include a peptide segment having the amino acid sequence of P₁P₂P₃P₄P₅P₆P₇P₈NDNP₁₂ where:
(i) P₁ is independently selected from an asparagine (N), a serine (S), or a threonine (T),
(ii) P₂ is independently selected from a serine (S) or a glycine (G),
(iii) P₃ is independently selected from a threonine (T), a glutamine (Q), an alanine (A), or glutamate (E),
(iv) P₄ is independently selected from a serine (S), a threonine (T), or an alanine (A),
(v) P₅ is independently selected from a glycine (G) or an alanine (A),
(vi) P₆ is independently selected from a glycine (G) or an alanine (A),
(vii) P₇ is independently selected from an alanine (A) or a serine (S),
(viii) P₈ is independently selected from a serine (S) or a threonine (T), and
(ix) P₁₂ is independently selected from a histidine (H), a threonine (T), or an alanine (A).

In some embodiments, a modified adeno-associated virus (AAV) capsid protein comprises a reference AAV capsid protein with one or more modifications to comprise a peptide segment within variable region I (VR I) of the reference AAV capsid protein, wherein the peptide segment has an amino acid sequence of P₁P₂P₃P₄P₅P₆P₇P₈P₉P₁₀P₁₁P₁₂, wherein positions P₉, P₁₀, and P₁₁, or a combination thereof, are not modified compared to a reference AAV capsid protein.

In some embodiments, a modified adeno-associated virus (AAV) capsid protein comprises a peptide segment within variable region I (VR I), wherein the peptide segment has an amino acid sequence of P₁P₂P₃P₄P₅P₆P₇P₈P₉P₁₀P₁₁P₁₂, wherein positions P₉, P₁₀, and P₁₁, or a combination thereof, are not modified compared to a reference AAV capsid protein.

In another aspect, the present disclosure provides a modified adeno-associated virus (AAV) capsid protein comprising a reference AAV capsid protein with one or more modifications to comprise a peptide segment within variable region I (VR I) of the reference AAV capsid protein, where the peptide segment has an amino acid sequence P₁P₂P₃P₄GGP₇P₈NDNP₁₂ (SEQ ID NO: 44921), wherein P₁, P₂, P₃, P₄, P₇, P₈, and P₁₂ are independently selected from any amino acid residue.

In another aspect, the present disclosure provides a modified adeno-associated virus (AAV) capsid protein comprising a peptide segment within variable region I (VR I), where the peptide segment has an amino acid sequence P₁P₂P₃P₄GGP₇P₈NDNP₁₂ (SEQ ID NO: 44921), wherein P₁, P₂, P₃, P₄, P₇, P₈, and P₁₂ are independently selected from any amino acid residue.

In some embodiments, the modified AAV capsid protein can comprise a peptide segment having the amino acid sequence of P₁P₂P₃P₄GGP₇P₈NDNP₁₂ (SEQ ID NO: 44921) where:
(i) P₂ is independently selected from an asparagine (N) or a serine (S);
(ii) P₂ is independently selected from a serine (S) or a glycine (G);
(iii) P₃ is independently selected from a threonine (T) or a glutamine (Q);
(iv) P₄ is independently selected from a serine (S), a threonine (T), or an alanine (A);
(v) P₇ is independently selected from an alanine (A) or a serine (S);
(vi) P₈ is independently selected from a serine (S) or a threonine (T); and
(vii) P₁₂ is independently selected from a histidine (H), a threonine (T), or an alanine (A).

In some embodiments, the modified AAV capsid protein can comprise a peptide segment having a sequence of P₁P₂TP₄GGP₇P₈NDNP₁₂ (SEQ ID NO: 44922), wherein P₁, P₂, P₄, P₇, P₈, and P₁₂ are independently selected from any amino acid residue.

In some embodiments, the modified AAV capsid protein can comprise a peptide segment has a sequence of P₁P₂QP₄GGP₇P₈NDNP₁₂ (SEQ ID NO: 44923), wherein P₁, P₂, P₄, P₇, P₈, and P₁₂ are independently selected from any amino acid residue.

In some embodiments, the modified AAV capsid protein can comprise a peptide segment having a sequence of P₁P₂TP₄GGP₇TNDNP₁₂ (SEQ ID NO: 44924), wherein P₁, P₂, P₄, P₇, and P₁₂ are independently selected from any amino acid residue.

In some embodiments, the modified AAV capsid protein can comprise a peptide segment has a sequence of NP₂TP₄GGP₇P₈NDNP₁₂ (SEQ ID NO: 44925), wherein P₂, P₄, P₇, P₈, and P₁₂ are independently selected from any amino acid residue.

In some embodiments, the modified AAV capsid protein can comprise a peptide segment has a sequence of SP₂TP₄GGP₇P₈NDNP₁₂ (SEQ ID NO: 44926), wherein P₂, P₄, P₇, P₈, and P₁₂ are independently selected from any amino acid residue.

In some embodiments, a modified adeno-associated virus (AAV) capsid protein includes a reference AAV capsid protein with one or more modifications to comprise a peptide segment within variable region I (VR I) of the reference AAV capsid protein, where the peptide segment has an amino acid sequence of P₁P₂P₃P₄P₅P₆P₇P₈P₉P₁₀P₁₁P₁₂, wherein positions P₅, P₆, or a combination thereof, are not modified compared to a reference AAV capsid protein. In some embodiments, a modified adeno-associated virus (AAV) capsid protein includes a reference AAV capsid protein with one or more modifications to comprise a peptide segment within variable region I (VR I) of the reference AAV capsid protein, where the peptide segment has an amino acid sequence of P₁P₂P₃P₄P₅P₆P₇P₈P₉P₁₀P₁₁P₁₂, wherein positions P₅, P₆, P₉, P₁₀, and P₁₁, or a combination thereof, are not modified compared to a reference AAV capsid protein.

In another aspect, the present disclosure provides a modified adeno-associated virus (AAV) capsid protein comprising a reference AAV capsid protein with one or more modifications to comprise a peptide segment within variable region I (VR I) of the reference AAV capsid protein, wherein the peptide segment has an amino acid sequence of NSTSGGP₇P₈NDNH (SEQ ID NO: 44927), wherein P₇ and P₈ are independently selected from any amino acid residue. In some embodiments, P₇ is independently selected from an alanine (A) or a serine (S) and P₈ is independently selected from a serine (S) or a threonine (T). In some embodiments, the modified adeno-associated virus (AAV) capsid protein comprises a peptide segment within variable region I (VR I), wherein the peptide segment has an amino acid sequence of NSTSGGP₇P₈NDNH (SEQ ID NO: 44927), wherein P₇ and P₈ are independently selected from any amino acid residue. In some embodiments, P₇ is independently selected from an alanine (A) or a serine (S) and P₈ is independently selected from a serine (S) or a threonine (T). In some embodiments, the peptide segment includes (i) NSTSGGASNDNH (SEQ ID NO: 46026), (ii) NSTSGGATNDNH (SEQ ID NO: 46029), (iii) NSTSGGSSNDNH (SEQ ID NO: 46031), (iv) NSTSGGSTNDNH (SEQ ID NO: 46034), or a corresponding sequence having one or more modifications (e.g., insertion, deletion, mutation, and/or substitution).

In another aspect, the present disclosure provides a modified adeno-associated virus (AAV) capsid protein comprising a reference AAV capsid protein with one or more modifications to comprise a peptide segment within variable region I (VR I) of the reference AAV capsid protein, wherein the peptide segment has an amino acid sequence of NSTTGGP₇P₈NDNH (SEQ ID NO: 44928), wherein P₇ and P₈ are independently selected from any amino acid residue. In some embodiments, P₇ is independently selected from an alanine (A) or a serine (S) and P₈ is independently selected from a serine (S) or a threonine (T). In some embodiments, the modified adeno-associated virus (AAV) capsid protein comprises a peptide segment within variable region I (VR I), wherein the peptide segment has an amino acid sequence of NSTTGGP₇P₈NDNH (SEQ ID NO: 44928), wherein P₇ and P₈ are independently selected from any amino acid residue. In some embodiments, P₇ is independently selected from an alanine (A) or a serine (S) and Pa is independently selected from a serine (S) or a threonine (T). In some embodiments, the peptide segment includes: (i) NSTTGGASNDNH (SEQ ID NO: 46073), (ii) NSTTGGATNDNH (SEQ ID NO: 46076), (iii) NSTTGGSSNDNH (SEQ ID NO: 46079), (iv) NSTTGGSTNDNH (SEQ ID NO: 46082), or a corresponding sequence having one or more modifications (e.g., insertion, deletion, mutation, and/or substitution).

In another aspect, the present disclosure provides a modified adeno-associated virus (AAV) capsid protein comprising a reference AAV capsid protein with one or more modifications to comprise a peptide segment within variable region I (VR I) of the reference AAV capsid protein, wherein the peptide segment has an amino acid sequence of SGQTGGP₇P₈NDNH (SEQ ID NO: 44929), wherein P₇ and P₈ are independently selected from any amino acid residue. In some embodiments, P₇ is independently selected from an alanine (A) or a serine (S) and P₈ is independently selected from a serine (S) or a threonine (T). In some embodiments, the modified adeno-associated virus (AAV) capsid protein comprises a peptide segment within variable region I (VR I), wherein the peptide segment has an amino acid sequence of SGQTGGP₇P₈NDNH (SEQ ID NO: 44929), wherein P₇ and P₈ are independently selected from any amino acid residue. In some embodiments, P₇ is independently selected from an alanine (A) or a serine (S) and Pa is independently selected from a serine (S) or a threonine (T). In some embodiments, the peptide segment includes: (i) SGQTGGASNDNH (SEQ ID NO: 46505), (ii) SGQTGGATNDNH (SEQ ID NO: 46508), (iii) SGQTGGSSNDNH (SEQ ID NO: 46511), (iv) SGQTGGSTNDNH (SEQ ID NO: 46514), or a corresponding sequence having one or more modifications (e.g., insertion, deletion, and/or substitution).

In another aspect, the present disclosure provides a modified adeno-associated virus (AAV) capsid protein comprising a reference AAV capsid protein with one or more modifications to comprise a peptide segment within variable region I (VR I) of the reference AAV capsid protein, wherein the peptide segment has an amino acid sequence of SGTAGGP₇P₈NDNT (SEQ ID NO: 44930), wherein P₇ and P₈ are independently selected from any amino acid residue. In some embodiments, P₇ is independently selected from an alanine (A) or a serine (S) and P₈ is independently selected from a serine (S) or a threonine (T). In some embodiments, the modified adeno-associated virus (AAV) capsid protein comprises a peptide segment within variable region I (VR I), wherein the peptide segment has an amino acid sequence of SGTAGGP₇P₈NDNT (SEQ ID NO: 44930), wherein P₇ and P₈ are independently selected from any amino acid residue. In some embodiments, P₇ is independently selected from an alanine (A) or a serine (S) and Pa is independently selected from a serine (S) or a threonine (T). In some embodiments, the peptide segment has a sequence of (i) SGTAGGASNDNT (SEQ ID NO: 46554), (ii) SGTAGGSSNDNT (SEQ ID NO: 46560), or a corresponding sequence having one or more modifications (e.g., insertion, deletion, and/or substitution). In some embodiments, the peptide segment does not comprise SGTAGGATNDNT (SEQ ID NO: 46557) or SGTAGGSINDNT (SEQ ID NO: 46563).

In another aspect, the present disclosure provides a modified adeno-associated virus (AAV) capsid protein comprising a reference AAV capsid protein with one or more modifications to comprise a peptide segment within variable region I (VR I) of the reference AAV capsid protein, wherein the peptide segment has an amino acid sequence of SGTSGGP₇P₈NDNA (SEQ ID NO: 44931), wherein P₇ and P₈ are independently selected from any amino acid residue. In some embodiments, P₇ is independently selected from an alanine (A) or a serine (S) and Pa is independently selected from a serine (S) or a threonine (T). In some embodiments, the modified adeno-associated virus (AAV) capsid protein comprises a peptide segment within variable region I (VR I), wherein the peptide segment has an amino acid sequence of SGTSGGP₇P₈NDNA (SEQ ID NO: 44931), wherein P₇ and P₈ are independently selected from any amino acid residue. In some embodiments, P₇ is independently selected from an alanine (A) or a serine (S) and Pa is independently selected from a serine (S) or a threonine (T). In some embodiments, the peptide segment includes: (i) SGTSGGASNDNA (SEQ ID NO: 46600), (ii) SGTSGGATNDNA (SEQ ID NO: 46600), (iii) SGTSGGSSNDNA (SEQ ID NO: 46603), (iv) SGTSGGSINDNA (SEQ ID NO: 46609), or a corresponding sequence having one or more modifications (e.g., insertion, deletion, and/or substitution).

In another aspect, the present disclosure provides a modified adeno-associated virus (AAV) capsid protein comprising a reference AAV capsid protein with one or more modifications to comprise a peptide segment within variable region I (VR I) of the reference AAV capsid protein, wherein the peptide segment has an amino acid sequence of SGTTGGP₇P₈NDNT (SEQ ID NO: 44932), wherein P₇ and P₈ are independently selected from any amino acid residue. In some embodiments, P₇ is independently selected from an alanine (A) or a serine (S) and P₈ is independently selected from a serine (S) or a threonine (T). In some embodiments, the modified adeno-associated virus (AAV) capsid protein comprises a peptide segment within variable region I (VR I) of the reference AAV capsid protein, wherein the peptide segment has an amino acid sequence of SGTTGGP₇P₈NDNT (SEQ ID NO: 44932), wherein P₇ and P₈ are independently selected from any amino acid residue. In some embodiments, P₇ is independently selected from an alanine (A) or a serine (S) and P₈ is independently selected from a serine (S) or a threonine (T). In some embodiments, the peptide segment includes: (i) SGTTGGASNDNT (SEQ ID NO: 46650), (ii) SGTTGGATNDNT (SEQ ID NO: 46653), (iii) SGTTGGSSNDNT (SEQ ID NO: 46656), (iv) SGTTGGSTNDNT (SEQ ID NO: 46659), or a corresponding sequence having one or more modifications (e.g., insertion, deletion, and/or substitution). In some embodiments, the peptide segment is SGTTGGSSNDNT (SEQ ID NO: 46656).

In another aspect, the present disclosure provides a modified adeno-associated virus (AAV) capsid protein comprising a reference AAV capsid protein with one or more modifications to comprise a peptide segment within variable region I (VR I) of the reference AAV capsid protein, wherein the peptide segment has an amino acid sequence of SSTAGGP₇P₈NDNA (SEQ ID NO: 44933), wherein P₇ and P₈ are independently selected from any amino acid residue. In some embodiments, P₇ is independently selected from an alanine (A) or a serine (S) and P₈ is independently selected from a serine (S) or a threonine (T). In some embodiments, the modified adeno-associated virus (AAV) capsid protein comprises a peptide segment within variable region I (VR I), wherein the peptide segment has an amino acid sequence of SSTAGGP₇P₈NDNA (SEQ ID NO: 44933), wherein P₇ and P₈ are independently selected from any amino acid residue. In some embodiments, P₇ is independently selected from an alanine (A) or a serine (S) and P₈ is independently selected from a serine (S) or a threonine (T). In some embodiments, the peptide segment includes: (i) SSTAGGASNDNA (SEQ ID NO: 47128), (ii) SSTAGGATNDNA (SEQ ID NO: 47131), (iii) SSTAGGSSNDNA (SEQ ID NO: 47134), (iv) SSTAGGSTNDNA (SEQ ID NO: 47137), or a corresponding sequence having one or more modifications (e.g., insertion, deletion, and/or substitution). In some embodiments, the peptide segment is SSTAGGASNDNA (SEQ ID NO: 47128). In some embodiments, the peptide segment is SSTAGGATNDNA (SEQ ID NO: 47131).

In some embodiments, the peptide segment is NSTSGASTNDNA (SEQ ID NO: 48390).

In some embodiments, the peptide segment is selected a peptide segment as shown in Table 20.

In some embodiments, a modified AAV capsid protein of the present disclosure can change the tropism, specificity and/or bio-distribution of an AAV comprising the modified AAV capsid protein. In some embodiments, an AAV comprising the modified AAV capsid protein has increased targeting to a target cell, tissue or organ when administered to a subject. In some embodiments, an AAV comprising the modified AAV capsid protein has decreased distribution outside of a target cell, tissue or organ when administered to a subject.

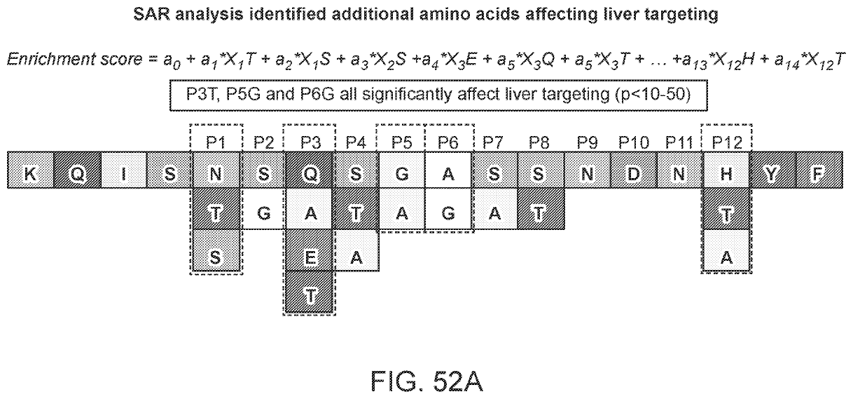

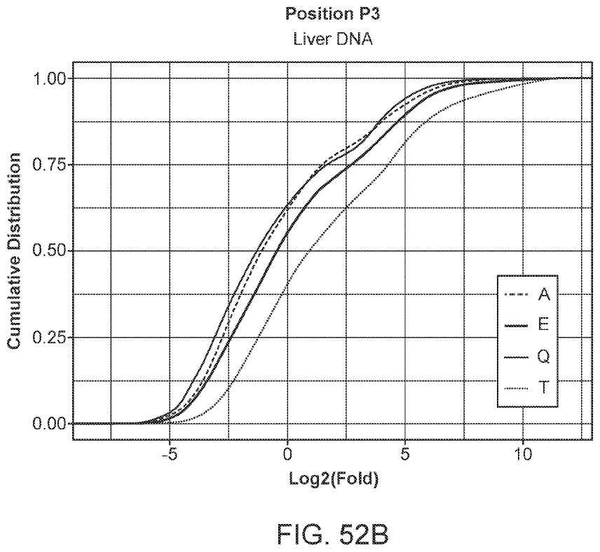

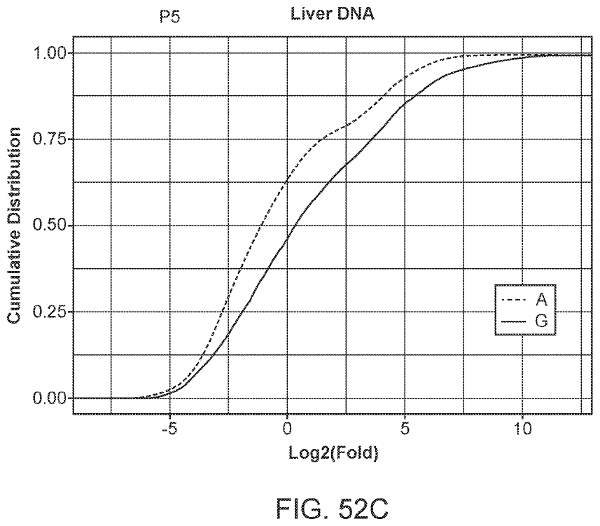

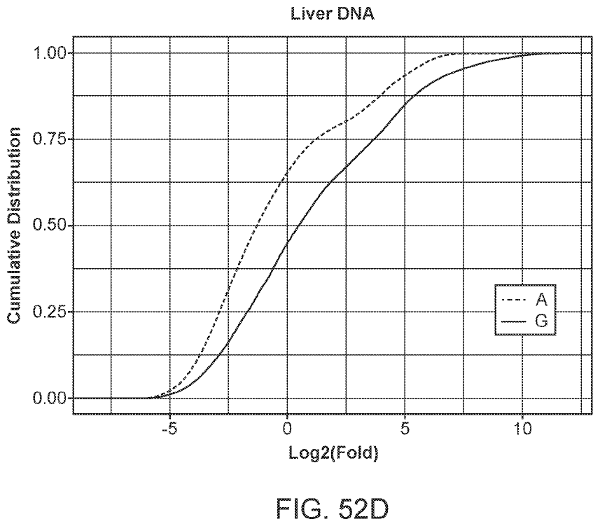

In some embodiments, tropism of a modified AAV capsid protein can be measured using an enrichment score. A non-limiting example of an enrichment score is based on the combination of amino acid residues present in the modified sequence within VR I, where the modified sequence includes P₁P₂P₃P₄P₅P₆P₇P₈P₉P₁₀P₁₁P₁₂. FIG. 52A provides an exemplary enrichment score formula, which is reproduced below.

$$Enrichment score = {a}_{0} + {a}_{1} {X}_{1} T + {a}_{2} {X}_{1} S + {a}_{3} {X}_{2} S + {a}_{4} {X}_{3} E + {a}_{5} {X}_{3} Q + {a}_{5} {X}_{3} T + … + {a}_{13} {X}_{12} H + {a}_{14} {X}_{12} T$$

where X₁ refers to, for example, amino acid position 1 (i.e., P₁), a refers to the metric calculated for the assigned amino acid residue at the amino acid position, and “T”, “S”, “E”, “Q”, “H”, and “T” refer to the amino acid residue at the X amino acid position.

### 6.2.6. Variable Region I (VR I)

In some embodiments, variable region I (VR I) corresponds to sequences between about position 259 to about position 275 in the modified AAV capsid protein. In some embodiments, variable region I (VR I) corresponds to sequences between about position 259 to about position 275 in the reference AAV capsid protein (e.g., any of the reference AAV capsid proteins described herein (sec. e.g., Section 4.2.4).

In some embodiments, a modified adeno-associated virus (AAV) capsid protein comprises a reference AAV capsid protein with one or more modifications to comprise a peptide segment within variable region I (VR I) of the reference AAV capsid protein

In some embodiments, the peptide segment is at a position between S261 and Y274 of an AAV9 capsid protein (SEQ ID NO: 61).

In some embodiments, the peptide segment is at a position c between S260 and Y273 of an Anc80 capsid protein (SEQ ID NO: 132).

In some embodiments, the peptide segment is at a position between S260 and Y273 of an Anc80L65 capsid protein (SEQ ID NO: 142).

In some embodiments, the peptide segment is at a position S260 and Y273 of an AAV2 capsid protein (SEQ ID NO: 55).

### 6.2.7. Other Modifications

In some embodiments, the modified AAV capsid protein comprises one or more additional modifications compared to a reference AAV capsid protein. The one or more additional modifications can be a insertion, deletion, substitution or a combination thereof, and can be located in the VRVIII or outside of the VRVIII.

In some embodiments, the modified AAV capsid protein is different from the reference AAV capsid protein by having one or more amino acid substitutions at a variable region of the reference AAV capsid protein. In some embodiments, the one or more amino acid substitutions is at a variable region, VR I, of the reference AAV capsid protein (FIG. 1).

The modified AAV capsid protein can be biologically or chemically produced. The modified AAV capsid protein can have tropism, specificity or localization different from the reference AAV capsid protein, particularly in liver, when administered to a mammalian subject. The mammalian subject can be a human, non-human primate (NHP), mice, rats, birds, rabbits, guinea pigs, hamsters, farm animals (including pigs and sheep), dogs, or cats.

In some embodiments, the modified AAV capsid protein comprises a sequence different from a reference AAV capsid protein by having an amino acid substitution at an amino acid position corresponding to position 266 in Anc80 VP1 and/or at an amino acid position corresponding to position 168 in Anc80 VP1.

In some embodiments, the modified AAV capsid protein comprises a sequence different from a reference AAV capsid protein by having (1) an alanine (A) amino acid residue at an amino acid position corresponding to position 266 in Anc80 VP1 or (2) a lysine (K) amino acid residue at an amino acid position corresponding to position 168 in Anc80 VP1. In some embodiments, the modified AAV capsid protein comprises a sequence different from a reference AAV capsid protein by having (1) an alanine (A) amino acid residue at an amino acid position corresponding to position 266 in Anc80 VP1 and (2) a lysine (K) amino acid residue at an amino acid position corresponding to position 168 in Anc80 VP1.

In some embodiments, the modified AAV capsid protein comprises a sequence different from a reference AAV capsid protein by having a glycine (G) amino acid residue at an amino acid position corresponding to position 266 in Anc80; or an arginine (R) amino acid residue at an amino acid position corresponding to position 168 in Anc80. In some embodiments, the modified AAV capsid protein comprises a sequence different from a reference AAV capsid protein by having a glycine (G) amino acid residue at an amino acid position corresponding to position 266 in Anc80; and an arginine (R) amino acid residue at an amino acid position corresponding to position 168 in Anc80.

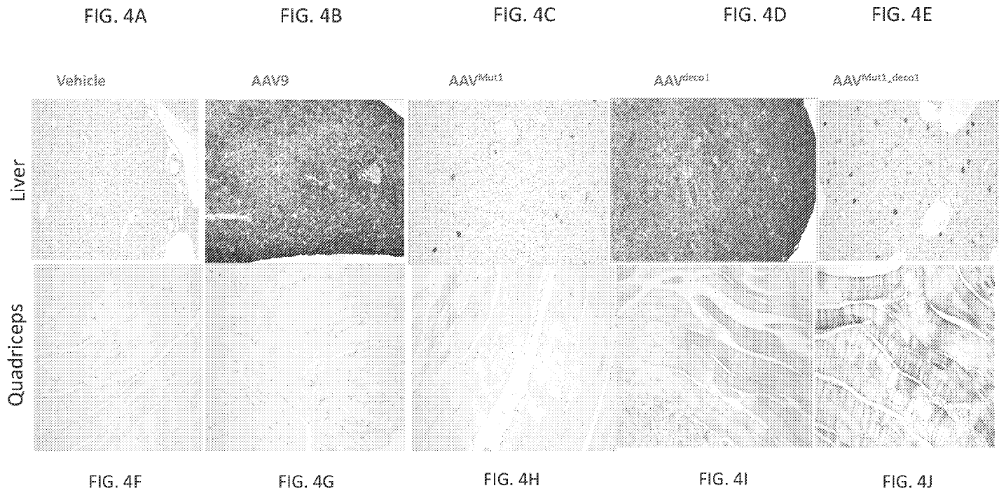

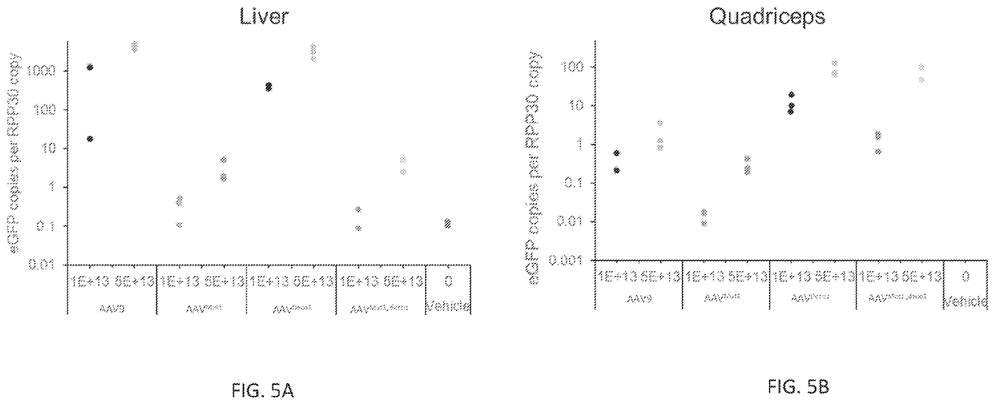

An amino acid position corresponding to position 266 in Anc80 VP1 and an amino acid position corresponding to position 168 in Anc80 VP1 in various VP1 protein sequences are indicated with boxes in FIGS. 3A-C and FIGS. 4A-D.

In some embodiments, the modified AAV capsid protein is different from a reference AAV capsid protein only at an amino acid position corresponding to position 266 in Anc80 VP1 or an amino acid position corresponding to position 168 in Anc80 VP1, other than the targeting peptide. In some embodiments, the modified AAV capsid protein is different from a reference AAV capsid protein only at two amino acid positions—an amino acid position corresponding to position 266 in Anc80 VP1 and an amino acid position corresponding to position 168 in Anc80 VP1, other than the targeting peptide. In some embodiments, the modified AAV capsid protein is different from a reference AAV capsid protein by more than the two amino acid substitutions.

In some embodiments, the modified AAV capsid protein comprises a sequence different from a reference AAV capsid protein by having an alanine (A) amino acid residue at an amino acid position corresponding to position 267 in AAV9 VP1 protein or a threonine (T) amino acid residue at an amino acid position corresponding to position 269 in AAV9 VP1. In some embodiments, the modified AAV capsid protein comprises a sequence different from a reference AAV capsid protein by having an alanine (A) amino acid residue at an amino acid position corresponding to position 267 in AAV9 VP1 protein and a threonine (T) amino acid residue at an amino acid position corresponding to position 269 in AAV9 VP1. In some embodiments, the modified AAV capsid protein is different from a reference AAV capsid protein only at an amino acid position corresponding to position 267 in AAV9 VP1 protein or an amino acid position corresponding to position 269 in AAV9 VP1, other than the targeting peptide. In some embodiments, the modified AAV capsid protein is different from a reference AAV capsid protein only at two amino acid positions—an amino acid position corresponding to position 267 in AAV9 VP1 protein and an amino acid position corresponding to position 269 in AAV9 VP1, other than the targeting peptide.

In some embodiments, a reference AAV capsid is an AAV capsid protein disclosed in WO2019/217911, which is incorporated by reference in its entirety herein.

In particular, an AAV capsid protein that is described therein to generate a “liver off” (“liver de-targeting”) AAV can be used in embodiments herein. In other embodiments, an AAV capsid protein that is described therein to generate a “liver on” (“liver targeting”) AAV can be used herein.

In some embodiments, two amino acid positions corresponding to position 266 and position 168 of Anc80 VP1 protein are used as liver-toggle positions. In some embodiments, AAV with a capsid protein having an alanine (A) amino acid residue at an amino acid position corresponding to position 266 in Anc80 VP1 or b) a lysine (K) amino acid residue at an amino acid position corresponding to position 168 in Anc80 VP1 exhibits liver-off phenotypes. In some embodiments, AAV with a capsid protein having a glycine (G) amino acid residue at an amino acid position corresponding to position 266 in Anc80 VP1 or b) an arginine (R) amino acid residue at an amino acid position corresponding to position 168 in Anc80 VP1 exhibits liver-on phenotypes.

In some embodiments, more than one toggle region residues are introduced to enhance the liver-off or the liver-on phenotypes. In some embodiments, a double mutant AAV9 G267A S269T is used.

In some embodiments, a reference AAV capsid is Anc80L65 capsid protein with a G266A mutation. In some embodiments, a reference AAV capsid is Anc80-55 capsid protein with a G266A mutation. In some embodiments, a reference AAV capsid is Anc80-129 capsid protein with a G266A mutation. In some embodiments, a reference AAV capsid is Anc80-156 capsid protein with a G266A mutation. In some embodiments, a reference AAV capsid is Anc80-751 capsid protein with a G266A mutation. In some embodiments, a reference AAV capsid is Anc80-1029 capsid protein with a G266A mutation. In some embodiments, a reference AAV capsid is Anc80-1712 capsid protein with a G266A mutation. In some embodiments, a reference AAV capsid is AAV9 capsid protein with a G267A mutation. In some embodiments, a reference AAV capsid is AAV9 capsid protein with G267A and S269T mutations.

In some embodiments, a reference AAV capsid comprises (1) an alanine (A) amino acid residue at an amino acid position corresponding to position 504 in AAV9; and (2) an alanine (A) amino acid residue at an amino acid position corresponding to position 505 in AAV9.

In some embodiments, the modified AAV capsid protein does not include one or more modifications as described in WO2019/217911 or WO 2021/050614. WO 2021/050614 showed that in a non-naturally occurring AAV capsid that A266 variants were less efficient in liver uptake compared to G266 variants. Amino acid position 266 from Anc80 in WO 2021/050614 corresponds to position 267 in AAV9 VP1. In some embodiments, the reference AAV capsid protein does not include an alanine (A) at an amino acid position corresponding to position 267 in AAV9 VP1. In some embodiments, the one or more modifications to a reference AAV capsid protein to comprise a peptide segment within variable region I (VR I) do not include modification to an alanine (A) at an amino acid position corresponding to position 267 in AAV9 VP1. In some embodiments, the reference AAV capsid protein comprises a glycine (G) at an amino acid position corresponding to position 267 in AAV9 VP1.

In some embodiments, the modified AAV capsid protein does not include an alanine (A) at an amino acid position corresponding to position 267 in AAV9 VP1 protein and a threonine (T) at an amino acid position corresponding to position 269 in AAV9 VP1. In some embodiments, the modified AAV capsid protein does not include an alanine (A) at an amino acid position corresponding to position 267 in AAV9 VP1 protein and a threonine (T) at an amino acid position corresponding to position 269 in AAV9 VP1.

### 6.2.8. Combinations of Targeting Peptides and Peptide Segments

In some embodiments, a modified AAV capsid protein comprises: (i) a targeting peptide at a site within VR VIII, wherein the targeting peptide has a sequence selected from SEQ ID NOs: 238-44858; and (ii) a peptide segment within variable region I (VR I), wherein the peptide segment as a sequence selected from SEQ ID NOs: 44935-47387.

In some embodiments, a modified AAV capsid protein comprises: (i) a targeting peptide at a site within VR VIII, wherein the targeting peptide has a sequence selected from SEQ ID NOs: 44864-44867, 44879-44883, 44911, 44912, 44913, 44918-44919, and 48391-157057; and (ii) a peptide segment within variable region I (VR I), wherein the peptide segment has a sequence selected from SEQ ID NOs: 46026, 46029, 46031, 46035, 46073, 46076, 46079, 46082, 46505, 46508, 46511, 46514, 46554, 46557, 46560, 46563, 46609, 46600, 46603, 46606, 46650, 46653, 46656, 46659, 47128, 47131, 47134, 47137, and 48390.

In some embodiments, the modified AAV capsid protein comprises an (i) a targeting peptide having an amino acid sequence selected from SEQ ID NOs 44864-44867, 44879-44883, 44911, 44912, 44913, 44918-44919, and 48391-157057; and (ii) a peptide segment that has the amino acid of sequence SGTAGGASNDNT (SEQ ID NO: 46554).

In some embodiments, the modified AAV capsid protein comprises an (i) a targeting peptide having an amino acid sequence selected from SEQ ID NOs: 44864-44867, 44879-44883, 44911, 44912, 44913, 44918-44919, and 48391-157057; and a peptide segment that has the amino acid sequence of SGTSGGSINDNA (SEQ ID NO: 46609).

In some embodiments, the modified AAV capsid protein comprises an (i a targeting peptide having an amino acid sequence selected from SEQ ID NOs: 44864-44867, 44879-44883, 44911, 44912, 44913, 44918-44919, and 48391-157057; and (ii) a peptide segment that has the amino acid sequence of SGTTGGSTNDNT (SEQ ID NO: 46659).

In some embodiments, the modified AAV capsid protein comprises an (i) a targeting peptide having an amino acid sequence selected from SEQ ID NOs: 44864-44867, 44879-44883, 44911, 44912, 44913, 44918-44919, and 48391-157057; and (ii) a peptide segment that has the amino acid sequence of SGTTGGSTNDNT (SEQ ID NO: 47128).

In some embodiments, the modified AAV capsid protein comprises an (i) a targeting peptide having an amino acid sequence selected from SEQ ID NOs: 44864-44867, 44879-44883, 44911, 44912, 44913, 44918-44919, and 48391-157057; and (ii) a peptide segment that has the amino acid sequence of SSTAGGATNDNA (SEQ ID NO: 47131).

In some embodiments, the modified AAV capsid protein comprises an (i) a targeting peptide having an amino acid sequence selected from SEQ ID NOs: 44864-44867, 44879-44883, 44911, 44912, 44913, 44918-44919, and 48391-157057; and (ii) a peptide segment that has the amino acid sequence of NSTSGGSSNDNA (SEQ ID NO: 48388).

In some embodiments, the modified AAV capsid protein comprises an (i) a targeting peptide having an amino acid sequence selected from SEQ ID NOs: 44864-44867, 44879-44883, 44911, 44912, 44913, 44918-44919, and 48391-157057; and (ii) a peptide segment that has the amino acid sequence of NSTSGASTNDNA (SEQ ID NO: 48390).

In some embodiments, a modified adeno-associated virus (AAV) capsid protein, comprising: a targeting peptide at a site within VR VIII, wherein the targeting peptide has a sequence selected from SEQ ID NOs: 48882, 44864, 44866, 44911, 44918, 98944-157057 and a peptide segment within VR I, wherein the peptide segment has a sequence selected from SEQ ID NOs: 48388, 48390, 46656, 47128, and 47131.

In some embodiments, a modified adeno-associated virus (AAV) capsid protein, comprising: a targeting peptide at a site within VR VIII, wherein the targeting peptide has a sequence selected from SEQ ID NOs: 44882, 44911, 44918, 49394, and 98944-144426 and a peptide segment within VR I, wherein the peptide segment has a sequence of NSTSGASTNDNA (SEQ ID NO: 48390).

In some embodiments, a modified adeno-associated virus (AAV) capsid protein, comprising: a targeting peptide at a site within VR VIII, wherein the targeting peptide has a sequence selected from SEQ ID NOs: 44880, 44886, 44864, and 144427-152004 and a peptide segment within VR I, wherein the peptide segment has a sequence of SGTTGGSSNDNT (SEQ ID NO: 46656).

In some embodiments, a modified adeno-associated virus (AAV) capsid protein, comprising: a targeting peptide at a site within VR VIII, wherein the targeting peptide has a sequence selected from SEQ ID NOs: 44864 and 152005-154530 and a peptide segment within VR I, wherein the peptide segment has a sequence of SSTAGGASNDNA (SEQ ID NO: 47128).

In some embodiments, a modified adeno-associated virus (AAV) capsid protein, comprising: a targeting peptide at a site within VR VIII, wherein the targeting peptide has a sequence selected from SEQ ID NOs: 44860 and 154531-157056 and a peptide segment within VR I, wherein the peptide segment has a sequence of SSTAGGATNDNA (SEQ ID NO: 47131).

In some embodiments, a modified adeno-associated virus (AAV) capsid protein, comprising: a targeting peptide at a site within VR VIII, wherein the targeting peptide has a sequence of selected from: SAQRGDRGQI (SEQ ID NO: 44911), SAQRGDHASW (SEQ ID NO: 157057), ENRRGDFNNT (SEQ ID NO: 44880), ENRRGDFNNL (SEQ ID NO: 44864), ENRRGDFQNT (SEQ ID NO: 44866), and SAQRGDLLLS (SEQ ID NO: 44882) and a peptide segment within VR I, wherein the peptide segment has a sequence of NSTSGGSSNDNA (SEQ ID NO: 48388).

### 6.3. Polynucleotides Encoding Modified AAV Capsid Proteins; Vectors; Host Cells

In another aspect, the present disclosure provides a polynucleotide encoding a modified AAV capsid protein described herein. In some embodiments, the polynucleotide is codon optimized for expression in a bacterial or mammalian cell.

In some embodiments, the polynucleotide is inserted into an expression vector. In some embodiments, the polynucleotide is operably linked to a promoter or a sequence inducing expression of a protein from the polynucleotide. The present disclosure provides a vector including the polynucleotide encoding a modified AAV capsid protein. The vector can be used for generation of the modified AAV capsid protein. In some embodiments, the vector is used to generate an AAV virion comprising the modified AAV capsid protein. In some embodiments, the vector further comprises an AAV rep protein or a fragment thereof. In some embodiments, the reference capsid protein for the modified AAV capsid protein and the rep protein are originated from an AAV of the same clade. In some embodiments, the reference capsid protein for the modified AAV capsid protein and the rep protein are originated from an AAV of different clades.

In some embodiments, the polynucleotide is transfected to a host cell. The present disclosure provides a host cell comprising the polynucleotide encoding a modified AAV capsid protein. The host cell can be a prokaryotic cell or eukaryotic cell. In some embodiments, the host cell is a mammalian cell or a yeast cell.

In some embodiments, the host cell further comprises another polynucleotide encoding an AAV protein. In some embodiments, the host cell comprises a functional rep gene; a recombinant nucleic acid vector comprising AAV inverted terminal repeats (ITRs) and an expressible polynucleotide; and sufficient helper functions to permit packaging of the recombinant nucleic acid vector into the modified AAV capsid protein.

In some embodiments, the components required for the host cell to package a recombinant nucleic acid vector in a modified AAV capsid protein are provided to the host cell in trans. In some embodiments, any one or more of the required components (e.g., a recombinant nucleic acid vector, rep sequences, cap sequences, and/or helper functions) are provided by a stable host cell which has been engineered to contain one or more of the required components using methods known to those of skill in the art. In some embodiments, such a stable host cell contains the required component(s) under the control of an inducible promoter. In some embodiments, the required component(s) is under the control of a constitutive promoter.

### 6.4. Recombinant Nucleic Acid Vector Containing an Expressible Polynucleotide

In one aspect, the present disclosure provides a recombinant nucleic acid vector containing an expressible polynucleotide. In some embodiments, the recombinant nucleic acid vector is encapsulated in the modified AAV capsid proteins disclosed herein. In preferred embodiments, the expressible polynucleotide comprises a transgene (in cis or trans configuration with other viral sequences).

The transgene can be, for example, a reporter gene (e.g., beta-lactamase, beta-galactosidase (LacZ), alkaline phosphatase, thymidine kinase, green fluorescent polypeptide (GFP), chloramphenicol acetyltransferase (CAT), or luciferase, or fusion polypeptides that include an antigen tag domain such as hemagglutinin or Myc), or a therapeutic gene (e.g., genes encoding hormones or receptors thereof, growth factors or receptors thereof, differentiation factors or receptors thereof, immune system regulators (e.g., cytokines and interleukins) or receptors thereof, enzymes, RNAs (e.g., inhibitory RNAs or catalytic RNAs), or target antigens (e.g., oncogenic antigens, autoimmune antigens). In some embodiments, the modified rAAV comprises an expressible polynucleotide encoding a therapeutic tRNA, miRNA, gene editing guide RNA, or RNA-editing guide RNA.

The transgene can be selected depending, at least in part, on the particular disease or deficiency being treated. Simply by way of example, gene transfer or gene therapy can be applied to the treatment of hemophilia, retinitis pigmentosa, cystic fibrosis, leber congenital amaurosis, lysosomal storage disorders, inborn errors of metabolism (e.g., inborn errors of amino acid metabolism including phenylketonuria, inborn errors of organic acid metabolism including propionic acidemia, inborn errors of fatty acid metabolism including medium-chain acyl-CoA dehydrogenase deficiency (MCAD)), cancer, achromatopsia, cone-rod dystrophies, macular degenerations (e.g., age-related macular degeneration), lipopolypeptide lipase deficiency, familial hypercholesterolemia, spinal muscular atrophy. Duchenne's muscular dystrophy, Alzheimer's disease, Parkinson's disease, obesity, inflammatory bowel disorder, diabetes, congestive heart failure, hypercholesterolemia, hearing loss, coronary heart disease, familial renal amyloidosis, Marfan's syndrome, fatal familial insomnia, Creutzfeldt-Jakob disease, sickle-cell disease, Huntington's disease, fronto-temporal lobar degeneration, Usher syndrome, lactose intolerance, lipid storage disorders (e.g., Niemann-Pick disease, type C), Batten disease, choroideremia, glycogen storage disease type II (Pompe disease), ataxia telangiectasia (Louis-Bar syndrome), congenital hypothyroidism, severe combined immunodeficiency (SCID), and/or amyotrophic lateral sclerosis (ALS). A transgene also can be, for example, an immunogen that is useful for immunizing a subject (e.g., a human, an animal (e.g., a companion animal, a farm animal, an endangered animal). For example, immunogens can be obtained from an organism (e.g., a pathogenic organism) or an immunogenic portion or component thereof (e.g., a toxin polypeptide or a by-product thereof). By way of example, pathogenic organisms from which immunogenic polypeptides can be obtained include viruses (e.g., picornavirus, enteroviruses, orthomyxovirus, reovirus, retrovirus), prokaryotes (e.g., Pneumococci, Staphylococci, Listeria, Pseudomonas), and eukaryotes (e.g., amebiasis, malaria, leishmaniasis, nematodes). It would be understood that the methods described herein and compositions produced by such methods are not to be limited by any particular transgene.

### 6.4.1. MTM1 Transgene

In certain embodiment, the transgene is the MTM1 transgene for treatment of subjects (preferably human subjects) suffering from XLMTM and/or carrying mutations in the MTM1 gene as described in PCT Application No. PCT/US2022/015842, which is herein incorporated by reference in its entirety.

A modified rAAV of the present disclosure can be administered to a subject in a suitable pharmaceutical carrier, e.g., as described herein, and as described in PCT Application No. PCT/US2022/015842, which is herein incorporated by reference in its entirety

Exemplary functional fragments of an MTM1 polypeptide include fragments comprising amino acids 29-486 of SEQ ID NO:165 (i.e., the amino acid sequence of SEQ ID NO: 164). Thus, in certain embodiments, the MTM1 polypeptides comprise amino acid residues 29-486 of SEQ ID NO: 165 or the amino acid sequence of SEQ ID NO:164.

In some embodiments, the MTM1 polypeptide comprises an amino acid sequence having at least 90%, at least 95%, at least 98%, at least 99% or 100% identity to a functional fragment of human MTM1 having the amino acid sequence of SEQ ID NO: 164. In some embodiments, the MTM1 polypeptide is a full length MTM1 polypeptide (e.g., a polypeptide of SEQ ID NO: 165)

In other embodiments, the MTM1 polypeptide is a fusion polypeptide comprising an amino acid sequence having at least at least 90%, at least 95%, at least 98%, at least 99% or 100% identity to SEQ ID NO: 164 fused to another polypeptide portion, e.g., one or more polypeptide portions that enhance one or more of in vivo stability, in vivo half-life, uptake/administration, and/or purification. In some embodiments, the polypeptide portion is an internalizing moiety.

In some embodiments, the MTM1 coding sequence comprises a nucleotide sequence having at least 80% sequence identity to SEQ ID NO: 166, which is of the native MTM1 coding sequence, or a portion thereof encoding a functional fragment of wild type MTM1, e.g., the functional fragment corresponding to amino acids 29-486 of MTM1 (SEQ ID NO: 164). In certain embodiments, the MTM1 coding sequence comprises a nucleotide sequence at least 80%, at least 85%, at least 90%, at least 95%, at least 97%, at least 98%, at least 99% or 100% identical to SEQ ID NO: 166 or a portion thereof encoding a functional fragment of wild type MTM1, e.g., the functional fragment corresponding to amino acids 29-486 of MTM1 (SEQ ID NO:164).

In other embodiments, the MTM1 coding sequence comprises a nucleotide sequence having at least 80% sequence identity to any of SEQ ID NOs: 167, 168 and 169, which are codon-optimized for expression in human cells, or to a portion of any of SEQ ID NOs: 167, 168 and 169 encoding a functional fragment of wild type MTM1, e.g., the functional fragment corresponding to amino acids 29-486 of MTM1 (SEQ ID NO: 164). In certain embodiments, the MTM1 coding sequence comprises a nucleotide sequence at least 80%, at least 85%, at least 90%, at least 95%, at least 97%, at least 98%, at least 99% or 100% identical to any one of SEQ ID NOs: 167, 168 and 169, or to or to a portion of any of SEQ ID NOs: 167, 168 and 169 encoding a functional fragment of wild type MTM1, e.g., the functional fragment corresponding to amino acids 29-486 of MTM1 (SEQ ID NO:164).

In some embodiments, the MTM1 coding sequence may further comprise a nucleotide sequence that encodes a linker and/or an internalizing moiety. In some embodiments, the internalizing moiety is an antibody or an antigen-binding fragment thereof.

### 6.4.2. Regulatory Sequences

The recombinant nucleic acid vector of the disclosure typically comprise regulatory sequences operably linked to expressible polynucleotide (e.g., the MTM1 coding sequence), for example, as described in PCT Application No. PCT/US2022/015842, which is herein incorporated by reference in its entirety. The regulatory sequence can comprise a promoter, an enhancer, and/or a repressor. In some embodiments, the regulatory sequence is tissue specific. For example, the regulatory sequence induces expression of a transgene in a specific target such as heart, liver, skeletal muscle, cardiac muscle, or other muscle.

Other suitable features of the rAAV include ITR sequences (e.g., wild type ITRs or a combination of wild type ITR sequences and an ITR sequence lacking a functional terminal resolution site, for example as set forth in SEQ ID NO: 178 and SEQ ID NO: 179), a intron (e.g., a chimeric intron comprising human herpesvirus beta and human globin 3 intronic sequences, for example as set forth in SEQ ID NO:176), a splice acceptor sequence 5′ of the MTM1 coding sequence (e.g., a human globin 3 splice acceptor sequence, for example as set forth in SEQ ID NO: 180), a polyadenylation sequence (e.g., a rabbit globin polyadenylation sequence, for example as set forth in SEQ ID NO: 177).

### 6.4.2.1 CAG Promoters

Certain embodiments of the present disclosure are based in part on the discovery that an ERE comprising a CAG promoter can drive far greater expression levels of the expressible polynucleotide (e.g., MTM1 coding sequence) than the desmin promoter in clinical development, for example, as described in PCT Application No. PCT/US2022/015842, which is herein incorporated by reference in its entirety. Without being bound by theory, it is believed the rAAV with the MTM1 coding sequence under the control of the CAG promoter can be therapeutically effective at lower doses than corresponding vectors in which the MTM1 coding sequence is under the control of the desmin promoter, and thus such vectors are believed to have improved therapeutic indexes as compared to corresponding vectors in which the MTM1 coding sequence is under the control of the desmin promoter.

In some embodiments, the CMV enhancer component of the CAG promoter or ERE comprises a sequence having at least 90%, at least 95%, at least 98%, at least 99% or 100% sequence identity to SEQ ID NO: 171.

In some embodiments, the chicken beta actin promoter component of the CAG promoter or ERE comprises a nucleotide sequence having at least 90%, at least 95%, at least 98%, at least 99% or 100% sequence identity to SEQ ID NO:172.

In some embodiments, the CAG promoter or ERE comprises a nucleotide sequence having at least 90%, at least 95%, at least 98%, at least 99% or 100% sequence identity to SEQ ID NO: 173.

An exemplary CAG ERE is used in the rAVE expression cassette (GeneDetect.com).

In some embodiments, the CAG ERE further comprises a chimeric intron, for example a chimeric intron formed from introns from the human betaherpes virus and rabbit beta globin. In some embodiments, the chimeric intron comprises a nucleotide sequence having at least 90%, at least 95%, at least 98%, at least 99% or 100% sequence identity to SEQ ID NO:174.

Further modifications of the CAG promoter can be used in the rAAV of the disclosure. For example, the intron in the 5′ untranslated region (UTR) of the CAG promoter can be truncated to accommodate larger inserts (Richardson et al., 2009, PLOS One, 4 (4), e5308. doi: 10.1371/journal.pone.0005308). Deletions in intron A of the hCMV promoter can also result in enhanced expression (Quilici et al., 2013, Biotechnol Lett. 35 (1), 21-27. doi: 10.1007/s10529-012-1043-z). Thus, a person skilled in the art could modify the CAG ERE or promoter sequences without compromising the high MTM1 expression levels observed with the constructs disclosed in Example 7.

### 6.4.2.2 Other Promoters

The rAAV of the disclosure may comprise, in lieu of a CAG ERE, an ERE comprising another constitutive promoter or a tissue specific or inducible promoter. Promoters that drive lower expression levels than a CAG promoter may be combined with other features that increase transgene expression (e.g., using codon optimized coding sequences) and/or reduce off target tropism of the virus (e.g., using muscle targeting and/or liver toggle capsid proteins).

In various embodiments the promoter is a constitutive, tissue-specific (e.g., muscle-specific) or inducible promoter. The promoters may be either naturally occurring promoters, or hybrid promoters that combine elements of more than one promoter.

In some embodiments, the promoter is a skeletal-muscle specific promoter. In some embodiments, the promoter is a cardiac-specific promoter.

Examples of constitutive promoters include, without limitation, a retroviral Rous sarcoma virus (RSV) LTR promoter (optionally with an RSV enhancer), a cytomegalovirus (CMV) promoter (optionally with a CMV enhancer), a SV40 promoter, a dihydrofolate reductase promoter, a β-actin promoter, a phosphoglycerol kinase (PGK) promoter, and a EF1α promoter.

Examples of tissue-specific promoters include, without limitation a synapsin-1 (Syn) promoter, a creatine kinase (MCK) promoter, a mammalian desmin (DES) promoter, an α-myosin heavy chain (a-MHC) promoter, or a cardiac Troponin T (cTnT) promoter.

Examples of inducible promoters include a zinc-inducible metallothionine (MT) promoter, a dexamethasone (Dex)-inducible mouse mammary tumor virus (MMTV) promoter, a tetracycline-inducible promoter, or a rapamycin-inducible promoter.

### 6.5. Modified AAV Virions

The present disclosure further provides a modified recombinant AAV (rAAV) virion comprising a modified AAV capsid protein described herein. In some embodiments, the modified rAAV comprises a modified AAV capsid protein and a recombinant nucleic acid vector.

In some embodiments, the modified rAAV comprising a modified AAV capsid protein achieves higher infection of a target following administration to a mammalian subject as compared to an rAAV comprising a corresponding reference AAV capsid protein. In some embodiments, the modified rAAV achieves higher expression in a target of an expressible polynucleotide within the recombinant nucleic acid vector following administration to a mammalian subject when compared to expression of the expressible polynucleotide administered in an rAAV comprising a corresponding reference AAV capsid protein.

In some embodiments, the modified rAAV comprising a modified AAV capsid protein achieves lower infection of an off-target following administration to a mammalian subject as compared to an rAAV comprising a corresponding reference AAV capsid protein. In some embodiments, the modified rAAV achieves lower expression in an off-target of an expressible polynucleotide within the recombinant nucleic acid vector following administration to a mammalian subject as compared to expression of the expressible polynucleotide administered in an rAAV comprising a corresponding reference AAV capsid protein. In typical embodiments, the corresponding reference AAV capsid protein is a capsid protein identical to the modified AAV capsid protein except that it does not include a targeting peptide and/or a liver-toggle mutation described above.

In some embodiments, the target is brain, muscle, spinal cord, eye, liver, muscle, or other organ. In some embodiments, the off-target tissue is brain, muscle, spinal cord, eye, liver, muscle, or other organ. In one embodiment, the target is muscle.

In some embodiments, the modified rAAV has less liver toxicity than an rAAV comprising a corresponding reference AAV capsid protein administered by the same route of administration and in the same dose. In some embodiments, the less liver toxicity is because of de-targeting of the modified rAAV to a liver.

### 6.6. Methods of Producing rAAV

The rAAV of the disclosure comprise a recombinant nucleic acid vector containing an expressible polynucleotide. In some embodiments, the expressible polynucleotide is operably linked to an ERE. The expressible polynucleotide and ERE optionally replace the AAV genomic coding region (e.g., replace the AAV rep and cap genes). The expressible polynucleotide and ERE are generally flanked on either side by AAV inverted terminal repeat (ITR) regions, although a single ITR may be sufficient to carry out the functions normally associated with configurations comprising two ITRs (see, for example, WO 94/13788), and vector constructs with only one ITR can thus be employed in conjunction with the rAAV of the present disclosure.

In some embodiments, the rAAV of the disclosure comprise an MTM1 coding sequence operably linked to an ERE. The MTM1 coding sequence and ERE optionally replace the AAV genomic coding region (e.g., replace the AAV rep and cap genes).

In order to replicate and package the vector, the missing functions are complemented with a packaging gene, or a plurality thereof, which together encode the necessary functions for the various missing rep and/or cap gene products. The packaging genes or gene cassettes are in one embodiment not flanked by AAV ITRs and in one embodiment do not share any substantial homology with the rAAV genome.

The rAAV vector construct, and the complementary packaging gene constructs can be implemented in a number of different forms. Viral particles, plasmids, and stably transformed host cells can all be used to introduce such constructs into the packaging cell, either transiently or stably.

In certain embodiments of this invention, the AAV vector and complementary packaging gene(s), if any, are provided in the form of bacterial plasmids, AAV particles, or any combination thereof. In other embodiments, either the AAV vector sequence, the packaging gene(s), or both, are provided in the form of genetically altered (preferably inheritably altered) eukaryotic cells. The development of host cells inheritably altered to express the AAV vector sequence, AAV packaging genes, or both, provides an established source of the material that is expressed at a reliable level.

A variety of different genetically altered cells can thus be used in the context of this invention. By way of illustration, a mammalian host cell may be used with at least one intact copy of a stably integrated rAAV vector. An AAV packaging plasmid comprising at least an AAV rep gene operably linked to a promoter can be used to supply replication functions (as described in U.S. Pat. No. 5,658,776). Alternatively, a stable mammalian cell line with an AAV rep gene operably linked to a promoter can be used to supply replication functions (see, e.g., WO 95/13392; WO 98/23018; and U.S. Pat. No. 5,656,785). The AAV cap gene, providing the encapsidation proteins as described above, can be provided together with an AAV rep gene or separately (see, e.g., the above-referenced patent documents as well as WO 98/27204.

Thus, the rAAV of the disclosure can be assembled by, for example, expression of its components in a packaging host cell. The components of a virus particle (e.g., rep sequences, cap sequences, inverted terminal repeat (ITR) sequences) can be introduced into a packaging host cell using one or more viral vectors.

Once assembled, rAAV particles can be purified, if desired, using routine methods. As used herein, “purified” virus particles refer to virus particles that are removed from components in the mixture in which they were made such as, but not limited to, viral components (e.g., rep sequences, cap sequences), packaging host cells, and partially- or incompletely-assembled virus particles.

### 6.7. Pharmaceutical Composition Comprising Modified rAAV

In one aspect, the present disclosure provides a pharmaceutical composition comprising a modified AAV capsid protein or a modified rAAV of the present disclosure and a pharmaceutically acceptable carrier. The modified rAAV can comprise a modified AAV capsid protein as described herein and a recombinant nucleic acid vector containing an expressible polynucleotide.

In particular embodiments, the present disclosure provides a pharmaceutical composition comprising an rAAV whose genome comprising an MTM1 coding sequence operably linked to an expression regulatory element (ERE); and one, two or all three of the following features: (a) the ERE is a hybrid expression regulatory element (ERE) comprising a CMV enhancer and a chicken beta actin promoter operably linked to the MTM1 coding sequence; and/or (b) the rAAV comprises a modified AAV capsid protein comprising at least one liver-toggle mutation and/or one muscle-targeting element; and/or (c) the MTM1 coding sequence is codon optimized for expression in human cells, optionally wherein the coding sequence has at least 90%, at least 95%, at least 98% or at least 99% sequence identity to any one of SEQ ID NOS: 167 to 170.

The pharmaceutical composition can be used to deliver the recombinant nucleic acid vector to a target within a mammalian subject. When the pharmaceutical composition is administered, the modified rAAV can achieve a higher infection of target cells following administration to a mammalian subject as compared to an rAAV comprising a corresponding reference AAV capsid protein administered by the same route of administration and in the same dose. In some embodiments, the modified rAAV achieves higher expression in target cells of an expressible polynucleotide within the recombinant nucleic acid genome following administration to a mammalian subject as compared to the expressible polynucleotide administered in an rAAV comprising a corresponding reference AAV capsid protein administered by the same route of administration and in the same dose.

The pharmaceutical composition can be formulated using one or more carriers, excipients, stabilizers and adjuvants to, for example: (1) increase stability; (2) increase cell transfection or transduction; (3) permit the sustained or delayed release; (4) alter the biodistribution (e.g., target the rAAV particle to specific tissues or cell types); (5) increase the translation of encoded protein in vivo; and/or (6) alter the release profile of encoded protein in vivo.

Formulations of the pharmaceutical compositions provided herein can include, without limitation, saline, which may be formulated with a variety of buffering solutions (e.g., phosphate buffered saline), lactose, sucrose, calcium phosphate, gelatin, dextran, agar, pectin, water, lipidoids, liposomes, lipid nanoparticles, polymers, lipoplexes, core-shell nanoparticles, peptides, proteins, nanoparticle mimics and combinations thereof.

Formulations of the pharmaceutical compositions described herein can be prepared by any method known or hereafter developed in the art of pharmacology. In general, such preparatory methods include the step of associating the active ingredient with a carrier and/or one or more other accessory ingredients (e.g., excipients, stabilizers and adjuvants).

A pharmaceutical composition in accordance with the present disclosure can be prepared, packaged, and/or sold in bulk, as a single unit dose, and/or as a plurality of single unit doses. As used herein, a unit dose refers to a discrete amount of the pharmaceutical composition including a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient (e.g., rAAV), the pharmaceutically acceptable carrier, and/or any additional ingredients in a pharmaceutical composition in accordance with the present disclosure can vary, depending upon the identity, size, and/or condition of the subject being treated and further depending upon the route by which the composition is to be administered.

Various carriers, excipients, stabilizers and adjuvants for formulating pharmaceutical compositions and techniques for preparing the composition are known in the art (see Remington: The Science and Practice of Pharmacy, 22nd Revised Ed., Pharmaceutical Press, 2012; incorporated herein by reference in its entirety). The use of suitable conventional carriers, excipients, stabilizers and adjuvants is contemplated within the scope of the present disclosure.

In some embodiments, the pharmaceutical composition is in the form of a solution containing concentrations of from about 1×101 to about 1×1016 genome copies (GCs)/ml of rAAV (e.g., a solution containing concentrations of from about 1×103 to about 1×1014 GCs/ml).

### 6.7.1. Routes of Administration

A modified rAAV of the present disclosure can be administered to a subject (e.g., a human or non-human mammal) in a suitable carrier. Suitable carriers include saline, which may be formulated with a variety of buffering solutions (e.g., phosphate buffered saline), lactose, sucrose, calcium phosphate, gelatin, dextran, agar, pectin, and water. A modified rAAV typically is administered in sufficient amounts to transduce or infect the desired cells and to provide sufficient levels of gene transfer and expression to provide a therapeutic benefit without undue adverse effects. Conventional and pharmaceutically acceptable routes of administration include, but are not limited to, direct delivery to an organ such as, for example, the muscle, liver or lung, orally, intranasally, intratracheally, intrathecally, intravenously, intramuscularly, intraocularly, subcutaneously, intradermally, or by other routes of administration. Routes of administration can be combined, if desired.

### 6.7.2. Dosages

The dose of a viral vector administered to a subject will depend primarily on factors such as the condition being treated, and the age, weight, and health of the subject. For example, a therapeutically effective dosage of a viral vector to be administered to a human subject generally is in the range of from about 0.1 ml to about 10 ml of a solution containing concentrations of from about 1×10¹ to about 1×10¹⁶ genome copies (GCs)/ml of viruses (e.g., a solution containing concentrations of from about 1×10³ to about 1×10¹⁴ GCs/ml). In some embodiments, the total dose of the rAAV administered to a subject is less than 3×10¹⁴ GCs, e.g., 1×10¹⁴ GCs or less, 5×10¹³ GCs or less, 1×10¹³ GCs or less, 5×10¹² GCs or less, or 1×10¹² GCs or less.

In another embodiment, a therapeutically effective dosage of a viral vector to be administered to a human subject generally is in the range of from about 0.1 ml to about 10 ml of a solution containing concentrations of from about 1×10¹ to 1×10¹² genome copies (GCs) of viruses (e.g., about 1×10³ to 1×10⁹ GCs). Transduction and/or expression of a transgene can be monitored at various time points following administration by DNA, RNA, or protein assays. In some instances, the levels of expression of the transgene can be monitored to determine the frequency and/or amount of dosage. Dosage regimens similar to those described for therapeutic purposes also may be utilized for immunization.

### 6.7.3. Targeting

Targeting of modified rAAVs can be tested in an experimental animal by measuring rAAV infection or expression of an expressible polynucleotide. In some embodiments, targeting is measured in a non-human primate (NHP), mice, rats, birds, rabbits, guinea pigs, hamsters, farm animals (including pigs and sheep), dogs, or cats.

Targeting of modified rAAVs can be measured after systemic or local administration of rAAVs. In some embodiments, targeting of modified rAAVs is measured after intravenous infusion of rAAVs.

### 6.7.3.1 RNA Data—Muscle:Liver Infection Ratio

In some embodiments, targeting of modified rAAVs is measured by measuring the ratio between the copy numbers of the transgene transcripts and housekeeping gene (e.g., RPP30) transcripts. In a particular embodiment, the transcripts are measured by RT-ddPCR. In some embodiments, the ratio is measured after a first administration into a mammal, e.g., a mouse, or a non-human primate such as a marmoset or rhesus macaque.

In some embodiments, a muscle:liver infection ratio (RNA) is measured by comparing the ratios between the copy numbers of the transgene transcripts and housekeeping gene (e.g., RPP30) transcripts in the two different organs (e.g., muscle v. liver).

$$muscle : liver infection ratio ( R N A ) = \frac{( \frac{transgene transcripts}{housekeeping transcripts} ) in muscle}{( \frac{transgene transcripts}{housekeeping transcripts} ) in liver}$$

In some embodiments, modified rAAV of the present disclosure provides a (transgene transcripts/housekeeping transcripts) ratio in liver of less than 1000, less than 900, less than 800, less than 700, less than 600, less than 500, less than 400, less than 300, less than 200, less than 100, less than 90, less than 80, less than 70, less than 60, less than 50, less than 40, less than 30, less than 20, or less than 10.

In certain embodiments, when the (transgene transcripts/housekeeping transcripts) in the liver is zero or below detection limits, the muscle:liver infection ratio is reported as >10,000 by convention.

In some embodiments, the modified rAAV of the present disclosure provides a muscle:liver infection ratio (RNA) of at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 150, at least 200, at least 500, at least 1000. In some embodiments, the muscle is triceps surae, biceps, heart or quadricep.

In some embodiments, modified rAAV of the present disclosure provides a muscle:liver infection ratio (RNA) of 1 to 10, 1 to 100, 10 to 20, 10 to 50, 10 to 80, 10 to 100, 20 to 100, 100 to 500, 100 to 1000, or 500 to 1000. In some embodiments, the muscle is triceps surae, bicep, heart or quadricep.

### 6.7.3.2 DNA Data—Muscle:Liver Infection Ratio

In some embodiments, targeting of modified rAAVs is measured by measuring the ratio between the copy numbers of the transgene DNA genomes to copy numbers of host genes or genetic loci (e.g., RPP30). In a particular embodiment, the genomes are measured by RT-ddPCR. In some embodiments, the ratio is measured after a first administration into a mammal, e.g., a mouse, or a non-human primate such as a marmoset or rhesus macaque.

In some embodiments, a muscle:liver infection ratio (DNA) is measured by comparing the ratios between the copy numbers of the transgene DNA genomes and housekeeping gene (e.g., RPP30) genomes in the two different organs (e.g., muscle v. liver).

$$muscle : liver infection ratio ( D N A ) = \frac{( \frac{transgene D N A genomes}{housekeeping genomes} ) in muscle}{( \frac{transgene D N A genomes}{housekeeping genomes} ) in liver}$$

In some embodiments, modified rAAV of the present disclosure provides a (transgene genomes/housekeeping genomes) ratio in liver of less than 1, or in a range from 1 to 10, 1 to 5, 1 to 2, 0.1 to 1, 0 to 1, 0.01 to 0.1, 0.01 to 0.5, or 0.01 to 0.05.

In certain embodiments, when the (transgene genomes/housekeeping genomes) in the liver is zero or below detection limits, the muscle:liver infection ratio is reported as >10,000 by convention.

In some embodiments, the modified rAAV of the present disclosure provides a muscle:liver infection ratio (DNA) of at least 1, at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 150, at least 200, at least 500, at least 1,000 or at least 10,000. In some embodiments, the muscle is triceps surae, biceps, heart or quadricep.

In some embodiments, modified rAAV of the present disclosure provides a muscle:liver infection ratio (DNA) in the range of 0.5 to 1, 0.5 to 5, 0.5 to 10, 1 to 10, 1 to 100, 2 to 8, 5 to 10, 10 to 20, 20 to 80, 10 to 50, 10 to 100, 50 to 80, 100 to 500, 100 to 1000, or 500 to 1000. In some embodiments, the muscle is triceps surae, biceps, heart, or quadricep. In some embodiments, the modified rAAV achieves a muscle:liver infection ratio (DNA) of at least 2, at least 5, at least 10, at least 20, at least 30, at least 40, at least 50, at least 60, at least 70, at least 80, at least 90, at least 100, at least 150, at least 200, at least 500, at least 1000. In some embodiments, the modified rAAV achieves a muscle:liver infection ratio of 0.1 to 1, 1 to 5, 1 to 10, 1 to 20, 1 to 50, 1 to 100, 1 to 200, 1 to 300, 100 to 500, 250 to 750, or 500 to 1000.

### 6.7.3.3 IHC Data Muscle:Liver Infection Ratio

In some embodiments, targeting of modified rAAVs is calculated using the % of cells that have been successfully transduced and express a transgene in a tissue (e.g., eGFP). In a particular embodiment, the transgene expression is measured by immunohistochemistry. In some embodiments, the ratio is measured after a first administration into a mammal, e.g., a mouse, or a non-human primate such as a marmoset or rhesus macaque.

In some embodiments, a muscle:liver infection ratio (IHC) is measured by comparing the ratios between the transgene % GFP+cells and housekeeping gene (e.g., RPP30) % GFP+cells in the two different organs (e.g., muscle v. liver).

$$muscle : liver infection ratio ( I H C ) = \frac{( \frac{transgene \% GFP + cells}{housekeeping \% GFP + cells} ) in muscle}{( \frac{transgene \% GFP + cells}{housekeeping \% GFP + cells} ) in liver}$$

In some embodiments, modified rAAV of the present disclosure provides a (transgene % GFP/housekeeping % GFP) ratio in liver of less than 1, less than 5, less than 10, or in a range from 1 to 10, 1 to 5, 1 to 2, 0.1 to 1, 0 to 1, 0.01 to 0.1, 0.01 to 0.5, or 0.01 to 0.05.

In certain embodiments, when the (transgene % GFP/housekeeping % GFP) in the liver is zero or below detection limits, the muscle:liver infection ratio is reported as >10,000 by convention.

In some embodiments, the modified rAAV of the present disclosure provides a muscle:liver infection ratio (IHC) of at least 1, at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 150, at least 200, at least 500, at least 1000. In some embodiments, the muscle is triceps surae, biceps, heart or quadricep.

In some embodiments, modified rAAV of the present disclosure provides a muscle:liver infection ratio (IHC) of 1 to 5, 1 to 10, 1 to 100, 2 to 8, 10 to 20, 20 to 30, 10 to 50, 10 to 100, 20 to 80, 50 to 80, 100 to 500, 100 to 1000, or 500 to 1000. In some embodiments, the muscle is triceps surae, bicep, heart or quadricep.

### 6.8. Method of Use

A modified rAAV as described herein can be used in research and/or therapeutic applications. In some embodiments, a modified rAAV is for genetically modifying a cell in vitro or in vivo. In some embodiments, a modified rAAV is used for gene therapy or for vaccination in a human or animal. More specifically, a modified rAAV can be used for gene addition, gene augmentation, genetic delivery of a polypeptide therapeutic, genetic vaccination, gene silencing, genome editing, gene therapy, RNAi delivery, cDNA delivery, mRNA delivery, miRNA delivery, miRNA sponging, genetic immunization, optogenetic gene therapy, transgenesis, DNA vaccination, or DNA immunization of liver cells or non-liver cells.

In some embodiments, a modified rAAV of the present disclosure is used for treatment of a muscle disease. In some embodiments, the disease is a muscular disease and/or the condition is muscle degeneration. In some embodiments, said muscular disease is a muscular dystrophy, a cardiomyopathy, a myotonia, a muscular atrophy, a myoclonus dystonia, a mitochondrial myopathy, a rhabdomyolysis, a fibromyalgia, and/or a myofascial pain syndrome. In some embodiments, the modified rAAV is used to deliver the rAAV to a striated muscle, preferably heart or a skeletal muscle or diaphragm.

In some embodiments, the rAAVs or pharmaceutical compositions described are useful in the treatment of subjects (preferably human subjects) suffering from XLMTM and/or carrying mutations in the MTM1 gene. “Treatment” of MTM encompasses a complete reversal or cure of the disease, or any range of improvement in conditions and/or adverse effects attributable to MTM. Merely to illustrate, “treatment” of MTM includes an improvement in any of the following effects associated with MTM or combination thereof: short life expectancy, respiratory insufficiency (partially or completely), poor muscle tone, drooping eyelids, poor strength in proximal muscles, poor strength in distal muscles, facial weakness with or without eye muscle weakness, abnormal curvature of the spine, joint deformities, and weakness in the muscles that control eye movement (ophthalmoplegia). Improvements in any of these conditions can be readily assessed according to standard methods and techniques known in the art.

A modified rAAV of the present disclosure can be administered to a subject in a suitable pharmaceutical carrier, e.g., as described in Section 4.7.

The rAAV of the disclosure are typically administered in sufficient amounts to transduce or infect the desired cells and to provide sufficient levels of gene transfer and expression to provide a therapeutic benefit to subjects suffering from a disease. In particular embodiments, the rAAV is administered in sufficient amounts to provide a therapeutic benefit to subjects suffering from XLMTM or carrying a mutation in the MTM1 gene, without undue adverse effects.

Conventional and pharmaceutically acceptable routes of administration include, but are not limited to, direct delivery to an organ such as, for example, the muscle, liver or lung, orally, intranasally, intratracheally, intrathecally, intravenously, intramuscularly, intraocularly, subcutaneously, intradermally, or by other routes of administration. Routes of administration can be combined, if desired.

The dose of a viral vector administered to a subject will depend primarily on factors such as the age, weight, and health (e.g., disease progression) of the subject. For example, a therapeutically effective dosage of a viral vector to be administered to a human subject generally is in the range of from about 0.1 ml to about 10 ml of a solution containing concentrations of from about 1×10¹ to about 1×10¹⁶ genome copies (GCs)/ml of viruses (e.g., a solution containing concentrations of from about 1×10³ to about 1×10¹⁴ GCs/ml). In some embodiments, the total dose of the rAAV administered to a subject is less than 3×10¹⁴ GCs, e.g., 1×10¹⁴ GCs or less, 5×10¹³ GCs or less, 1×10¹³ GCs or less, 5×10¹² GCs or less, or 1×10¹² GCs or less.

Transduction and/or expression of the transgene can be monitored at various time points following administration by DNA, RNA, or protein assays.

Accordingly, the present disclosure provides a method of treating and/or preventing a muscular disease and/or muscle degeneration by administering a modified rAAV described herein.

In some embodiments, the modified rAAV capsid of the present disclosure is used to deliver a transgene to a target tissue. In some embodiments the target tissue is cardiac muscle, diaphragm, tibialis anterior, quadriceps, biceps femoris, tibia gastrocnemius, tibialis anterior, triceps brachii, heart-ventricle wall, heart-atria, other skeletal muscle, or any muscle tissue described herein.

In some embodiments, the target tissue is heart. In some embodiments, the target tissue is liver.

### 7. ADDITIONAL EMBODIMENTS

### 7.1. Embodiment A

Embodiment A1. A modified adeno-associated virus (AAV) capsid protein, wherein: the capsid protein comprises: a targeting peptide within variable region (VR) VIII, wherein the targeting peptide has the sequence X₁X₂X₃RGDX₇X₈X₉X₁₀ and X₁, X₂, X₃, X₇, X₈, X₉ and X₁₀ are each independently selected from any amino acid residue.

Embodiment A2. The modified AAV capsid protein of Embodiment A1, wherein the AAV capsid protein has one or more modifications comprising amino acid insertions, deletions, substitutions, or combinations thereof as compared to a reference AAV capsid protein.

Embodiment A3. The modified AAV capsid protein of any one of Embodiment A1-A2, wherein the targeting peptide is at a site within variable region (VR) VIII.

Embodiment A4. The modified AAV capsid protein of any one of Embodiment A2-A3, wherein the one or more modifications comprises an amino acid insertion, deletion, substitution, or a combination thereof to introduce the targeting peptide into VR VIII of the reference AAV capsid protein.

Embodiment A5. The modified AAV capsid protein of any one of Embodiment A2-A4, wherein the one or more modifications comprises an amino acid modification outside of VR VIII of the reference AAV capsid protein.

Embodiment A6. The modified AAV capsid protein of any one of Embodiments A2-A5, having at least 90%, 95%, 98%, 99% or 99.5% sequence identity to the sequence of the reference AAV capsid protein.

Embodiment A7. The modified AAV capsid protein of any one of Embodiments A1-A6, wherein X₇, X₈, X₉ and X₁₀ are independently selected from A, D, E, F, G, H, I, K, L, M, N, Q, R, S, T, V, and Y.

Embodiment A8. The modified AAV capsid protein of any one of Embodiments A2-A7, wherein X₁, X₂, and X₃ are independently selected from any amino acid residue.

Embodiment A9. The modified AAV capsid protein of Embodiment A8, wherein X₁, X₂, and X₃ are amino acids identical to the amino acids at corresponding positions of the reference AAV capsid protein.

Embodiment A10. The modified AAV capsid protein of Embodiment A8, wherein X₁ is an amino acid that is identical to the amino acid at a corresponding position of the reference AAV capsid protein.

Embodiment A11. The modified AAV capsid protein of Embodiment A8, wherein X₂ is an amino acid that is identical to the amino acid at a corresponding position of the reference AAV capsid protein.

Embodiment A12. The modified AAV capsid protein of Embodiment A8, wherein X₃ is an amino acid that is identical to the amino acid at a corresponding position of the reference AAV capsid protein.

Embodiment A13. The modified AAV capsid protein of Embodiment A8, wherein X₁ and X₃ are amino acids that are identical to the amino acids at corresponding positions of the reference AAV capsid protein.

Embodiment A14. The modified AAV capsid protein of Embodiment A8, wherein X₁ and X₂ are amino acids that are identical to the amino acids at corresponding positions of the reference AAV capsid protein.

Embodiment A15. The modified AAV capsid protein of Embodiment A8, wherein X₂ and X₃ are amino acids that are identical to the amino acids at corresponding positions of the reference AAV capsid protein.

Embodiment A16. The modified AAV capsid protein of Embodiment A8, wherein X₁, X₂, and X₃ are amino acids that are not identical to the amino acids at corresponding positions of the reference AAV capsid protein.

Embodiment A17. The modified AAV capsid protein of Embodiment A8, wherein X₁ is an amino acid that is not identical to the amino acid at a corresponding position of the reference AAV capsid protein.

Embodiment A18. The modified AAV capsid protein of Embodiment A8, wherein X₂ is an amino acid that is not identical to the amino acid at a corresponding position of the reference AAV capsid protein.

Embodiment A19. The modified AAV capsid protein of Embodiment A8, wherein X₃ is an amino acid that is not identical to the amino acid at a corresponding position of the reference AAV capsid protein.

Embodiment A20. The modified AAV capsid protein of Embodiment A8, wherein X₁ and X₃ are amino acids that are not identical to the amino acids at corresponding positions of the reference AAV capsid protein.

Embodiment A21. The modified AAV capsid protein of Embodiment A8, wherein X₁ and X₂ are amino acids that are not identical to the amino acids at corresponding positions of the reference AAV capsid protein.

Embodiment A22. The modified AAV capsid protein of Embodiment A8, wherein X₂ and X₃ are amino acids that are not identical to the amino acids at corresponding positions of the reference AAV capsid protein.

Embodiment A23. The modified AAV capsid protein of any one of Embodiment A2-A22, wherein the modified AAV capsid protein comprises one or more substitutions, one or more insertions, one or more deletions, or a combination thereof into VR VIII of the reference AAV capsid protein.

Embodiment A24. The modified AAV capsid protein of Embodiment A23, wherein one or more modifications comprises a substitution of one or more amino acids between amino acid positions 565 and 595 of the reference AAV capsid.

Embodiment A25. The modified AAV capsid protein of any one of Embodiments A1-A24, wherein X₁ is selected from S, E, A, D, N, Q, or T.

Embodiment A26. The modified AAV capsid protein of any one of Embodiments A1-A24, wherein X₂ is selected from N, A, D, E, F, G, H, I, K, L, M, N, P, Q, R, S, T, V, W, or Y.

Embodiment A27. The modified AAV capsid protein of any one of Embodiments A1-A24, wherein X₃ is selected from R, Q, A, D, E, F, G, H, I, K, L, M, N, P, S, T, V, W, or Y.

Embodiment A28. The modified AAV capsid protein of any one of Embodiments A1-A24, wherein: X₂ is N; and X₃ is R.

Embodiment A29. The modified AAV capsid protein of any one of Embodiments A1-A28, wherein: X₁ is E; X₂ is N; and X₃ is R.

Embodiment A30. The modified AAV capsid protein of any one of Embodiments A1-A28, wherein: X₁ is S; X₂ is N; and X₃ is R.

Embodiment A31. The modified AAV capsid protein of any one of Embodiments A1-A30, wherein RGDX₇X₈X₉X₁₀ has an amino acid sequence selected from SEQ ID NOs.: 238-44858.

Embodiment A32. The modified AAV capsid protein of any one of Embodiments A1-A30, wherein RGDX₇X₈X₉X₁₀ has an amino acid sequence selected from SEQ ID NOs.: 238-248.

Embodiment A33. The modified AAV capsid protein of any one of Embodiments A1-A30 wherein RGDX₇X₈X₉X₁₀ has an amino acid sequence selected from SEQ ID NOs.: 238-338.

Embodiment A34. The modified AAV capsid protein of any one of Embodiments A1-A30, wherein RGDX₇X₈X₉X₁₀ has an amino acid sequence selected from SEQ ID NOs.: 238-438.

Embodiment A35. The modified AAV capsid protein of any one of Embodiments A1-A30, wherein RGDX₇X₈X₉X₁₀ has an amino acid sequence selected from SEQ ID NOs.: 238-538.

Embodiment A36. The modified AAV capsid protein of any one of Embodiments A1-A30, wherein RGDX₇X₈X₉X₁₀ has an amino acid sequence selected from SEQ ID NOs.: 238-638.

Embodiment A37. The modified AAV capsid protein of any one of Embodiments A1-A30, wherein RGDX₇X₈X₉X₁₀ has an amino acid sequence selected from SEQ ID NOs.: 238-738.

Embodiment A38. The modified AAV capsid protein of any one of Embodiments A1-A30, wherein RGDX₇X₈X₉X₁₀ has an amino acid sequence selected from SEQ ID NOs.: 3881, 12092, 14601, 15342, 21498, and 31396.

Embodiment A39. The modified AAV capsid protein of any one of Embodiments A1-A30, wherein RGDX₇X₈X₉X₁₀ has the amino acid sequence of SEQ ID NO: 238.

Embodiment A40. The modified AAV capsid protein of any one of Embodiments A1-A30, wherein X₁X₂X₃RGDX₇X₈X₉X₁₀ (SEQ ID NO: 44915) has an amino acid sequence selected from SEQ ID NOs.: 44859-44883, 44911, 44912, 44913, 44918-44919, and 44921-95452.

Embodiment A41. The modified AAV capsid protein of any one of Embodiments A1-A30, wherein X₁X₂X₃RGDX₇X₈X₉X₁₀ (SEQ ID NO: 44915) has an amino acid sequence selected from SEQ ID NOs.: 44864-44865, 44867, and 44879-44883.

Embodiment A42. The modified AAV capsid protein of any one of Embodiments A1-A30, wherein X₁X₂X₃RGDX₇X₈X₉X₁₀ has an amino acid sequence selected from SEQ ID NOs.: 44859-44878, 44911, 44912, 44913, and 44918-44919.

Embodiment A43. The modified AAV capsid protein of any one of Embodiments A1-A30, wherein the X₇ is selected from R, F, H, L, Q, R, and Y.

Embodiment A44. The modified AAV capsid protein of any one of Embodiments A43, wherein the X₇ is R.

Embodiment A45. The modified AAV capsid protein of Embodiment A43 or A44, wherein X₈ is selected from S, G, D, I, L, N, Q, T, and V.

Embodiment A46. The modified AAV capsid protein of Embodiment A45, wherein X₈ is S or G.

Embodiment A47. The modified AAV capsid protein of any one of Embodiments A1-A30, A45 or A46, wherein X₉ is selected from any of the amino acids.

Embodiment A48. The modified AAV capsid protein of any one of Embodiment A47, wherein X₉ is selected from V, S, N, G, Q, L, T, and Y.

Embodiment A49. The modified AAV capsid protein of any one of Embodiments A1-A30 and A47-A48, wherein X₁₀ is selected from I, V, S, L, M, R, T, and Q.

Embodiment A50. The modified AAV capsid protein of Embodiment A49, wherein X₁₀ is I or V.

Embodiment A51. The modified AAV capsid protein of any one of Embodiments A1-A30 and A45-A48, wherein RGDRX₈X₉X₁₀ has an amino acid sequence selected from: RGDRSX₉I, RGDRGX₉I, RGDRSX₉V, or RGDRGX₉V.

Embodiment A52. The modified AAV capsid protein of any one of Embodiments A1-A30 and A45-A48, wherein RGDRX₈X₉X₁₀ has an amino acid sequence selected from: RGDRGVX₁₀ (SEQ ID NO: 157058), RGDRGSX₁₀ (SEQ ID NO: 157059), RGDRGNX₁₀ (SEQ ID NO: 157060), RGDRGGX₁₀ (SEQ ID NO: 157061), RGDRGQX₁₀ (SEQ ID NO: 157062), RGDRGX₉V (SEQ ID NO: 157063), RGDRGX₉I (SEQ ID NO: 157064). RGDRGX₉S (SEQ ID NO. 157065), RGDRGX₉L (SEQ ID NO: 157066), RGDRGX₉Q (SEQ ID NO: 157067), RGDHX₈X₉L (SEQ ID NO: 157068), RGDRX₈X₉I (SEQ ID NO: 157069), RGDRX₈X₉V (SEQ ID NO: 157070), RGDRX₈X₉L (SEQ ID NO: 157071), RGDYX₈X₉L (SEQ ID NO: 157072), RGDYX₈X₉V (SEQ ID NO. 157073). RGDYX₈X₉M (SEQ ID NO: 157074), and RGDLX₈X₉T (SEQ ID NO: 157075).

Embodiment A53. The modified AAV capsid protein of any one of Embodiments A1-A30 and A47-A50, wherein the modified AAV capsid protein does not comprise an amino acid sequence selected from: RGDRMVF, RGDRTVI, SRGDRPM, ISLRGDR, and RGDLLLS (SEQ ID NO: 1).

Embodiment A54. The modified AAV capsid protein of any one of Embodiments A1-A30 and A43-A50, wherein the targeting peptide has a sequence selected from: X₁X₂X₃RGDHVNL (SEQ ID NO: 98924); X₁X₂X₃RGDLIGR (SEQ ID NO: 98925); X₁X₂X₃RGDQSTL (SEQ ID NO: 98926); X₁X₂X₃RGDRGQI (SEQ ID NO: 98927); X₁X₂X₃RGDRGVV (SEQ ID NO: 98928); X₁X₂X₃RGDRQGI (SEQ ID NO: 98929); X₁X₂X₃RGDRSQT (SEQ ID NO: 98930); X₁X₂X₃RGDRSVV (SEQ ID NO: 98931); X₁X₂X₃RGDLLLS (SEQ ID NO: 98932); X₁X₂X₃RGDFNNL (SEQ ID NO: 98933); X₁X₂X₃RGDFQNT (SEQ ID NO: 98934); X₁X₂X₃RGDLTVT (SEQ ID NO: 98935); X₁X₂X₃RGDYVGL (SEQ ID NO: 98936); X₁X₂X₃RGDYSSV (SEQ ID NO: 98937); X₁X₂X₃RGDHGVL (SEQ ID NO: 98938); X₁X₂X₃RGDRDYL (SEQ ID NO: 98939); X₁X₂X₃RGDYNSL (SEQ ID NO: 98940); X₁X₂X₃RGDYTSV (SEQ ID NO: 98941); X₁X₂X₃RGDYTSM (SEQ ID NO: 98942); and X₁X₂X₃RGDFNNT (SEQ ID NO: 98943).

Embodiment A55. The modified AAV capsid protein of any one of Embodiments A1-A54, wherein the reference AAV capsid protein is selected from VP1, VP2 and VP3.

Embodiment A56. The modified AAV capsid protein of any one of Embodiments A2-A55, wherein the reference AAV capsid protein is a capsid protein of an AAV selected from the group consisting of; AAV9; Anc8065; Anc80-55, Anc80-129, Anc80-156, Anc80-751, Anc80-1029, Anc80-1712, AAV2; AAV1; AAV6; AAV3; AAV LK03; AAV7; AAV8; AAV hu.37; AAV rh.10; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu.9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L33; Anc80L36; Anc80L44; Anc80L1; Anc110; and Anc80DI.

Embodiment A57. The modified AAV capsid protein of any one of Embodiments A2-A56, wherein the reference AAV capsid protein is a capsid protein having a sequence selected from SEQ ID Nos: 54-152, 44885-44898, 44916-44917, or a fragment thereof.

Embodiment A58. The modified AAV capsid protein of any one of Embodiments A2-A57, wherein the reference AAV capsid protein is a capsid protein having a sequence of SEQ ID NO: 61 or a fragment thereof.

Embodiment A59. The modified AAV capsid protein of any one of Embodiments A2-A57, wherein the reference AAV capsid protein is a capsid protein having a sequence of SEQ ID NO: 142 or a fragment thereof.

Embodiment A60. The modified AAV capsid protein of any one of Embodiments A2-A57, wherein the targeting peptide is positioned between 565 and 595 within VR VIII of the modified AAV capsid protein.

Embodiment A61. The modified AAV capsid protein of any one of Embodiments A2-A60, wherein: (i) the reference AAV capsid protein is a capsid protein of AAV1 or a modification thereof and the targeting peptide is between Q585 and T589 of the reference AAV capsid protein; (ii) the reference AAV capsid protein is a capsid protein of AAV2 or a modification thereof and the targeting peptide is between Q584 and R588 or between N587 and R588 of the reference AAV capsid protein; (iii) the reference AAV capsid protein is a capsid protein of AAV3 or a modification thereof and the targeting peptide is between Q585 and T589 of the reference AAV capsid protein; (iv) the reference AAV capsid protein is a capsid protein of AAV4 or a modification thereof and the targeting peptide is between G581 and N585 of the reference AAV capsid protein; (v) the reference AAV capsid protein is a capsid protein of AAV5 or a modification thereof and the targeting peptide is between Q574 and T578 of the reference AAV capsid protein; (vi) the reference AAV capsid protein is a capsid protein of AAV6 or a modification thereof and the targeting peptide is between Q585 and T589 of the reference AAV capsid protein; (vii) the reference AAV capsid protein is a capsid protein of AAV7 or a modification thereof and the targeting peptide is between Q586 and T590 of the reference AAV capsid protein; (viii) the reference AAV capsid protein is a capsid protein of AAV8 or a modification thereof and the targeting peptide is between Q587 and A591 of the modified AAV capsid protein; (ix) the reference AAV capsid protein is a capsid protein of AAV9 or a modification thereof and the targeting peptide is between H587 and A591 of the reference AAV capsid protein; (x) the reference AAV capsid protein is a capsid protein of AAVrh10 or a modification thereof and the targeting peptide is between Q587 and A591 of the reference AAV capsid protein; (xi) the reference AAV capsid protein is a capsid protein of AAVpo.1 or a modification thereof and the targeting peptide is between N564 and S568 of the reference AAV capsid protein; (xii) the reference AAV capsid protein is a capsid protein of AAV12 or a modification thereof and the targeting peptide is between N589 and A593 of the reference AAV capsid protein; (xiii) the reference AAV capsid protein is a capsid protein of Anc80 or a modification thereof and the targeting peptide is between Q585 and T589 of the reference AAV capsid protein; (xiv) the reference AAV capsid protein is a capsid protein of Anc80L65 or a modification thereof and the targeting peptide is between Q585 and T589 of the reference AAV capsid protein; (xv) the reference AAV capsid protein is a capsid protein of Anc80-55 or a modification thereof and the targeting peptide is between Q585 and T589 of the reference AAV capsid protein; (xvi) the reference AAV capsid protein is a capsid protein of Anc80-129 or a modification thereof and the targeting peptide is between Q585 and T589 of the reference AAV capsid protein; (xvii) the reference AAV capsid protein is a capsid protein of Anc80-156 or a modification thereof and the targeting peptide is between Q585 and T589 of the reference AAV capsid protein; (xviii) the reference AAV capsid protein is a capsid protein of Anc80-751 or a modification thereof and the targeting peptide is between Q585 and T589 of the reference AAV capsid protein; (xix) the reference AAV capsid protein is a capsid protein of Anc80-1029 or a modification thereof and the targeting peptide is between Q585 and T589 of the reference AAV capsid protein; or (xx) the reference AAV capsid protein is a capsid protein of Anc80-1712 or a modification thereof and the targeting peptide is between Q585 and T589 of the reference AAV capsid protein.

Embodiment A62. The modified AAV capsid protein of any one of Embodiments A2-A60, wherein: (i) the reference AAV capsid protein is a capsid protein of AAV1 or a modification thereof and the targeting peptide is between D590 and P591 or between S588 and T589 of the reference AAV capsid protein; (ii) the reference AAV capsid protein is a capsid protein of AAV2 or a modification thereof and the targeting peptide is between R588 and Q589 or between N587 and R588 of the reference AAV capsid protein; (iii) the reference AAV capsid protein is a capsid protein of AAV3 or a modification thereof and the targeting peptide is between S586 and S587 or between N588 and T589 of the reference AAV capsid protein; (iv) the reference AAV capsid protein is a capsid protein of AAV4 or a modification thereof and the targeting peptide is between S584 and N585 or between S586 and N587 of the reference AAV capsid protein; (v) the reference AAV capsid protein is a capsid protein of AAV5 or a modification thereof and the targeting peptide is between S575 and S576 or between T577 and T578 of the reference AAV capsid protein; (vi) the reference AAV capsid protein is a capsid protein of AAV6 or a modification thereof and the targeting peptide is between D590 and P591 or S588 and T589 of the reference AAV capsid protein; (vii) the reference AAV capsid protein is a capsid protein of AAV7 or a modification thereof and the targeting peptide is between N589 and T590 of the reference AAV capsid protein; (viii) the reference AAV capsid protein is a capsid protein of AAV8 or a modification thereof and the targeting peptide is between N590 and T591 of the modified AAV capsid protein; (ix) the reference AAV capsid protein is a capsid protein of AAV9 or a modification thereof and the targeting peptide is between Q588 and A589 of the reference AAV capsid protein; (x) the reference AAV capsid protein is a capsid protein of AAVrh10 or a modification thereof and the targeting peptide is between N590 and A591 of the reference AAV capsid protein; (xi) the reference AAV capsid protein is a capsid protein of AAVpo.1 or a modification thereof and the targeting peptide is between N567 and S568 or between N569 and T570 of the reference AAV capsid protein; (xii) the reference AAV capsid protein is a capsid protein of AAV12 or a modification thereof and the targeting peptide is between N592 and A593 or between T594 and T595 of the reference AAV capsid protein; (xiii) the reference AAV capsid protein is a capsid protein of Anc80 or a modification thereof and the targeting peptide is between T589 and A590 or between N587 and T588 of the reference AAV capsid protein; (xiv) the reference AAV capsid protein is a capsid protein of Anc80L65 or a modification thereof and the targeting peptide is between T589 and A590 or between N587 and T588 of the reference AAV capsid protein; (xv) the reference AAV capsid protein is a capsid protein of Anc80-55 or a modification thereof and the targeting peptide is between T589 and A590 or between N587 and T588 of the reference AAV capsid protein; (xvi) the reference AAV capsid protein is a capsid protein of Anc80-129 or a modification thereof and the targeting peptide is between T589 and A590 or between N587 and T588 of the reference AAV capsid protein; (xvii) the reference AAV capsid protein is a capsid protein of Anc80-156 or a modification thereof and the targeting peptide is between T589 and A590 or between N587 and T588 of the reference AAV capsid protein; (xviii) the reference AAV capsid protein is a capsid protein of Anc80-75 J or a modification thereof and the targeting peptide is between T589 and A590 or between N587 and T588 of the reference AAV capsid protein; (xix) the reference AAV capsid protein is a capsid protein of Anc80-1029 or a modification thereof and the targeting peptide is between T589 and A590 or between N587 and T588 of the reference AAV capsid protein; or (xx) the reference AAV capsid protein is a capsid protein of Anc80-1712 or a modification thereof and the targeting peptide is between T589 and A590 or between N587 and T588 of the reference AAV capsid protein.

Embodiment A63. The modified AAV capsid protein of any one of Embodiments A1-A55, having the sequence selected from SEQ ID NOs.: 44900-44909.

Embodiment A64. The modified AAV capsid protein of any one of Embodiments A2-A63, wherein the reference AAV capsid protein is a liver-toggle mutant of a capsid protein of an AAV variant selected from the group consisting of: AAV9; Anc80L65; Anc80-55. Anc80-129, Anc80-156, Anc80-751, Anc80-1029, Anc80-1712, AAV2; AAV1; AAV6; AAV3; AAV LK03; AAV7; AAV8; AAV hu.37; AAV rh.10; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu.9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L33; Anc80L36; Anc80L44; Anc80L1; Anc110; and Anc80DI.

Embodiment A65. The modified AAV capsid protein of any one of Embodiments A2-A64, wherein the reference AAV capsid protein is a liver-toggle mutant of a capsid protein having a sequence selected from SEQ ID Nos: 54-152, 44885-44898, 44916-44917, or a fragment thereof.

Embodiment A66. The modified AAV capsid protein of any one of Embodiments A63-A65, comprising: an alanine (A) amino acid residue at an amino acid position corresponding to position 266 in Anc80; or a lysine (K) amino acid residue at an amino acid position corresponding to position 168 in Anc80.

Embodiment A67. The modified AAV capsid protein of any one of Embodiments A63-A65, wherein the reference AAV capsid protein is a liver toggle mutant of a capsid protein of AAV9 comprising an alanine (A) amino acid residue at an amino acid position 267 and a threonine (T) amino acid residue at an amino acid position 269.

Embodiment A68. The modified AAV capsid protein of any one of Embodiments A2-A67, wherein the reference AAV capsid protein is a capsid protein having a sequence of SEQ ID NO: 163.

Embodiment A69. The modified protein of any one of Embodiments A63-A65, wherein the reference AAV capsid protein is a liver toggle mutant of a capsid protein of Anc80L65.

Embodiment A70. The modified protein of Embodiment A69, wherein the capsid protein of Anc80L65 comprises the sequence of SEQ ID NO: 142.

Embodiment A71. The modified protein of any one of Embodiments A64-A70, wherein the reference AAV capsid protein is a capsid protein selected from: Anc80-55, Anc80-129, Anc80-156, Anc80-751, Anc80-1029, and Anc80-1712.

Embodiment A72. The modified protein of Embodiment A71, wherein the reference AAV capsid protein is a capsid protein having a sequence selected from SEQ ID NOs: 44885-44898, and 44916-44917.

Embodiment A73. A polynucleotide encoding the modified AAV capsid protein of any one of Embodiments A1 to A72.

Embodiment A74. A vector comprising the polynucleotide of Embodiment A73.

Embodiment A75. The vector of Embodiment A74, further comprising a promoter operably linked to the polynucleotide.

Embodiment A76. A host cell comprising the modified AAV capsid protein of any one of Embodiments A1 to A72, the polynucleotide specified in Embodiment A73, or the vector of Embodiment A74 or A75.

Embodiment A77. A recombinant AAV virion (rAAV) comprising the modified AAV capsid protein of any one of Embodiments A1 to A72.

Embodiment A78. The AAV virion of Embodiment A77, further comprising an exogenous polynucleotide.

Embodiment A79. The AAV virion of Embodiment A78, wherein the exogenous polynucleotide comprises a template for homology directed repair.

Embodiment A80. The AAV virion of Embodiment A78, wherein the exogenous polynucleotide comprises an expressible polynucleotide encoding a therapeutic tRNA, miRNA, gene editing guide RNA, or RNA-editing guide RNA.

Embodiment A81. The AAV virion of Embodiment A78, wherein the exogenous polynucleotide comprises an expressible polynucleotide encoding a therapeutic protein.

Embodiment A82. The AAV virion of Embodiment A81, wherein the therapeutic protein is MTM1 or a fragment thereof.

Embodiment A83. The AAV virion of Embodiment A82, wherein the expressible polypeptide comprises the sequence of SEQ ID NO: 165 or a fragment thereof.

Embodiment A84. The AAV virion of Embodiment A82, wherein the expressible polypeptide comprises the sequence having at least 80%, 85%, 90%, 95%, 98%, 99% or 100% sequence identity to any of SEQ ID Nos: 166-170.

Embodiment A85. The AAV virion of any one of Embodiments A78-A84, wherein the exogenous polynucleotide further comprises a regulatory sequence.

Embodiment A86. The AAV virion of Embodiment A85, wherein the regulatory sequence comprises expression regulatory elements (EREs).

Embodiment A87. The AAV virion of Embodiment A86, wherein the EREs comprise a CAG promoter.

Embodiment A88. The AAV virion of Embodiment A86, wherein the EREs comprise a sequence having at least 90%, at least 95%, at least 98%, at least 99% or 100% sequence identity to any one of SEQ IDs NO: 171-173.

Embodiment A89. A pharmaceutical composition comprising the AAV virion of any one of Embodiments A77-A88.

Embodiment A90. A method for treating or ameliorating or preventing a disease or condition in a subject, comprising administering a therapeutically effective amount of the AAV virion of Embodiment A77 or the pharmaceutical composition of claim 89.

Embodiment A91. The method of treating or ameliorating or preventing a disease according to Embodiment A90, wherein the disease is a muscular disease and/or the condition is muscle degeneration.

Embodiment A92. The method of treating or ameliorating or preventing a disease according to Embodiment A91, wherein said muscle is a striated muscle, preferably heart or a skeletal muscle or diaphragm.

Embodiment A93. The method of treating or ameliorating or preventing a disease according to Embodiment A92, wherein said muscular disease is a muscular dystrophy, a cardiomyopathy, a myotonia, a muscular atrophy, a myoclonus dystonia, a mitochondrial myopathy, a rhabdomyolysis, a fibromyalgia, and/or a myofascial pain syndrome.

Embodiment A94. The modified adeno-associated virus (AAV) capsid protein of any one of Embodiments A1-A72, for use in treating and/or preventing a muscular disease and/or muscle degeneration.

Embodiment A95. An AAV virion comprising the modified AAV capsid protein of any one of Embodiments A1-A72 or an AAV virion of any one of Embodiments 77-88 for use in treating and/or preventing a muscular disease and/or in muscle regeneration.

Embodiment A96. A pharmaceutical composition comprising the modified AAV capsid protein of any one of Embodiments A1 to A72, and/or the AAV virion specified in any one of Embodiments A77-A88 for use in treating and/or preventing a muscular disease and/or in muscle regeneration.

Embodiment A97. A method of transferring an exogenous polynucleotide into a muscle cell, comprising the step of administering the AAV virion specified in any one of Embodiments A77-A88 to a subject.

Embodiment A98. The method of Embodiment A97, wherein the administration results in transfer of the exogenous polynucleotide in the muscle cell, at a muscle:liver infection ratio of greater than 1 when measured by genome copies of the AAV virion.

Embodiment A99. The method of Embodiment A97, wherein the muscle:liver infection ratio ranges from 1 to 100.

Embodiment A100. The method of Embodiment A98, wherein the muscle:liver infection ratio ranges from 1 to 10.

Embodiment A101. The method of Embodiment A100, wherein the muscle:liver infection ratio ranges from 2 to 8.

Embodiment A102. The method of any one of Embodiments A97-A101, wherein the administration results in expression of the exogenous polynucleotide in the muscle cell, at a muscle:liver expression ratio of greater than 10.

Embodiment A103. The method of Embodiment A102, wherein the muscle:liver expression ratio ranges from 10 to 100.

Embodiment A104. The method of Embodiment A103, wherein the muscle:liver expression ratio ranges from 20 to 80.

Embodiment A105. The method of any one of Embodiments A97-A104, wherein the muscle:liver expression ratio ranges from 50 to 80 when measured by mRNA transcript expression.

Embodiment A106. The method of any one of Embodiments A97-A105, wherein the muscle:liver expression ratio ranges from 10 to 50 when measured by protein expression.

Embodiment A107. The method of any one of Embodiments A97-A106, wherein the muscle cell is selected from triceps surae, biceps, heart and quadricep.

Embodiment A108. Use of the AAV capsid polypeptide of any one of Embodiments A1 to A72, and/or the AAV virion specified in any one of Embodiments A77-A88 for transferring an exogenous polynucleotide into a muscle cell.

Embodiment A109. The use according to Embodiment A108, wherein said use is a non-therapeutic use, preferably wherein said use is an in vitro use.

Embodiment A110. The use according to Embodiment A108, wherein the muscle cell is selected from triceps surae, biceps, heart and quadricep.

### 7.2. Embodiment B

Embodiment B1. A modified adeno-associated virus (AAV) capsid protein, comprising a peptide segment within variable region I (VR I) having an amino acid sequence of P₁P₂P₃P₄P₅P₆P₇P₈NDNP₁₂, wherein P₁, P₂, P₃, P₄, P₅, P₆, P₇, Pa, and Pin are independently selected from any amino acid residue.

Embodiment B2. The modified AAV capsid protein of Embodiment B1, wherein (i) P₁ is independently selected from an asparagine (N), a serine (S), or a threonine (T); (ii) P₂ is independently selected from a serine (S) or a glycine (G); (iii) P₃ is independently selected from a threonine (T), a glutamine (Q), an alanine (A), or glutamate (E); (iv) P₄ is independently selected from a serine (S), a threonine (T), or an alanine (A); (v) P₅ is independently selected from a glycine (G) or an alanine (A); (vi) P₆ is independently selected from a glycine (G) or an alanine (A); (vii) P₇ is independently selected from an alanine (A) or a serine (S); (viii) P₈ is independently selected from a serine (S) or a threonine (T); and (ix) P₁₂ is independently selected from a histidine (H), a threonine (T), or an alanine (A).

Embodiment B3. The modified AAV capsid protein of Embodiment B1 or B2, wherein P₅, P₆ or both P₅ and P₆ are not an alanine (A).

Embodiment B4. The modified AAV capsid protein of Embodiment B1 or B2, wherein the peptide segment does not comprise an alanine (A) at P₆ and a threonine (T) at Pa.

Embodiment B5. The modified AAV capsid protein of any one of Embodiments B1-B4, wherein the peptide segment has a sequence of P₁P₂P₃P₄GGP₇P₈NDNP₁₂ (SEQ ID NO: 44921), wherein P₁, P₂, P₃, P₄, P₇, Pa, and Pre are independently selected from any amino acid residue.

Embodiment B6. The modified AAV capsid protein of Embodiment B5, wherein (i) P₁ is independently selected from an asparagine (N) or a serine (S); (ii) P₂ is independently selected from a serine (S) or a glycine (G); (iii) P₃ is independently selected from a threonine (T) or a glutamine (Q); (iv) P₄ is independently selected from a serine (S), a threonine (T), or an alanine (A); (v) P₇ is independently selected from an alanine (A) or a serine (S); (vi) P₈ is independently selected from a serine (S) or a threonine (T); and (vii) P₁₂ is independently selected from a histidine (H), a threonine (T), or an alanine (A).

Embodiment B7. The modified AAV capsid protein of Embodiment B5, wherein the peptide segment has a sequence of P₁P₂TP₄GGP₇P₈NDNP₁₂ (SEQ ID NO: 44922), wherein P₁, P₂, P₄, P₇, P₈, and P₁₂ are independently selected from any amino acid residue.

Embodiment B8. The modified AAV capsid protein of Embodiment B5, wherein the peptide segment has a sequence of P₁P₂QP₄GGP₇P₈NDNP₁₂ (SEQ ID NO: 44923), wherein P₁, P₂, P₄, P₇, P₈, and P₁₂ are independently selected from any amino acid residue.

Embodiment B9. The modified AAV capsid protein of Embodiment B5, wherein the peptide segment has a sequence of P₁P₂TP₄GGP₇TNDNP₁₂ (SEQ ID NO: 44924), wherein P₁, P₂, P₄, P₇, and P₁₂ are independently selected from any amino acid residue.

Embodiment B10. The modified AAV capsid protein of Embodiment B5, wherein the peptide segment has a sequence of NP₂TP₄GGP₇P₈NDNP₁₂ (SEQ ID NO: 44925), wherein P₂, P₄, P₇, P₈, and P₁₂ are independently selected from any amino acid residue.

Embodiment B11. The modified AAV capsid protein of Embodiment B5, wherein the peptide segment has a sequence of SP₂TP₄GGP₇P₈NDNP₁₂ (SEQ ID NO: 44926), wherein P₂, P₄, P₇, P₈, and P₁₂ are independently selected from any amino acid residue.

Embodiment B12. The modified AAV capsid protein of any one of Embodiments B1-B6, wherein the peptide segment is NSTSGGP₇P₈NDNH (SEQ ID NO: 44927), wherein P₇ and P₈ are independently selected from any amino acid residue.

Embodiment B13. The modified AAV capsid of protein of Embodiment B12, wherein P₇ is independently selected from an alanine (A) or a serine (S) and P₈ is independently selected from a serine (S) or a threonine (T).

Embodiment B14. The modified AAV capsid protein of Embodiment B12 or B13, wherein the peptide segment comprises: (i) NSTSGGASNDNH (SEQ ID NO: 46026), (ii) NSTSGGATNDNH (SEQ ID NO: 46029), (iii) NSTSGGSSNDNH (SEQ ID NO: 46031), or (iv) NSTSGGSTNDNH (SEQ ID NO: 46034).

Embodiment B15. The modified AAV capsid protein of any one of Embodiments B1-6, wherein the peptide segment is NSTTGGP₇P₈NDNH (SEQ ID NO: 44928), wherein P₇ and P₈ are independently selected from any amino acid residue.

Embodiment B16. The modified AAV capsid protein of Embodiment B15, wherein P₇ is independently selected from an alanine (A) or a serine (S) and P₈ is independently selected from a serine (S) or a threonine (T).

Embodiment B17. The modified AAV capsid protein of Embodiment B15 or B16, wherein the peptide segment comprises: (i) NSTTGGASNDNH (SEQ ID NO: 46073), (ii) NSTTGGATNDNH (SEQ ID NO: 46076), (iii) NSTTGGSSNDNH (SEQ ID NO: 46079), or (iv) NSTTGGSTNDNH (SEQ ID NO: 46082).

Embodiment B18. The modified AAV capsid protein of any one of Embodiments B1-B6, wherein the peptide segment is SGQTGGP₇P₈NDNH (SEQ ID NO: 44929), wherein P₇ and P₈ are independently selected from any amino acid residue.

Embodiment B19. The modified AAV capsid protein of Embodiment B18, wherein P₇ is independently selected from an alanine (A) or a serine (S) and P₈ is independently selected from a serine (S) or a threonine (T).

Embodiment B20. The modified AAV capsid protein of Embodiment B18 or B19, wherein the peptide segment comprises: (i) SGQTGGASNDNH (SEQ ID NO: 46505), (ii) SGQTGGATNDNH (SEQ ID NO: 46508). (iii) SGQTGGSSNDNH (SEQ ID NO: 46511), or (iv) SGQTGGSTNDNH (SEQ ID NO: 46514).

Embodiment B21. The modified AAV capsid protein of any one of Embodiments B1-B6, wherein the peptide segment is SGTAGGP₇P₈NDNT (SEQ ID NO: 44930), wherein P₇ and P₈ are independently selected from any amino acid residue.

Embodiment B22. The modified AAV capsid protein of Embodiment B21, wherein P₇ is independently selected from an alanine (A) or a serine (S) and P₈ is independently selected from a serine (S) or a threonine (T).

Embodiment B23. The modified AAV capsid protein of Embodiment B21 or B22, wherein the peptide segment comprises: (i) SGTAGGASNDNT (SEQ ID NO: 46554), or (ii) SGTAGGSSNDNT (SEQ ID NO: 46560).

Embodiment B24. The modified AAV capsid protein of Embodiment B21 or B22, wherein the peptide segment does not comprise SGTAGGATNDNT (SEQ ID NO: 46557) or SGTAGGSTNDNT (SEQ ID NO: 46563).

Embodiment B25. The modified AAV capsid protein of any one of Embodiments B1-6, wherein the peptide segment is SGTSGGP₇P₈NDNA (SEQ ID NO: 44931), wherein P₇ and P₈ are independently selected from any amino acid residue.

Embodiment B26. The modified AAV capsid protein of Embodiment B25, wherein P₇ is independently selected from an alanine (A) or a serine (S) and P₈ is independently selected from a serine (S) or a threonine (T).

Embodiment B27. The modified AAV capsid protein of Embodiment B25 or B26, wherein the peptide segment comprises: (i) SGTSGGASNDNA (SEQ ID NO: 46600), (ii) SGTSGGATNDNA (SEQ ID NO: 46603), (iii) SGTSGGSSNDNA (SEQ ID NO: 46606), or (iv) SGTSGGSTNDNA (SEQ ID NO: 46609).

Embodiment B28. The modified AAV capsid protein of any one of Embodiments B1-B6, wherein the peptide segment is SGTTGGP₇P₈NDNT (SEQ ID NO: 44932), wherein P₇ and P₈ are independently selected from any amino acid residue.

Embodiment B29. The modified AAV capsid protein of Embodiment B28, wherein P₇ is independently selected from an alanine (A) or a serine (S) and P₈ is independently selected from a serine (S) or a threonine (T).

Embodiment B30. The modified AAV capsid protein of Embodiment B28 or B29, wherein the peptide segment comprises: (i) SGTTGGASNDNT (SEQ ID NO: 46650), (ii) SGTTGGATNDNT (SEQ ID NO: 46653), (iii) SGTTGGSSNDNT (SEQ ID NO: 46656), or (iv) SGTTGGSTNDNT (SEQ ID NO: 46659).

Embodiment B31. The modified AAV capsid protein of any one of Embodiments B1-B6, wherein the peptide segment is SSTAGGP₇P₈NDNA (SEQ ID NO: 44933), wherein P₇ and P₈ are independently selected from any amino acid residue.

Embodiment B32. The modified AAV capsid protein of Embodiment B31, wherein P₇ is independently selected from an alanine (A) or a serine (S) and P₈ is independently selected from a serine (S) or a threonine (T).

Embodiment B33. The modified AAV capsid protein of Embodiment B31 or B32, wherein the peptide segment comprises: (i) SSTAGGASNDNA (SEQ ID NO: 47128), (ii) SSTAGGATNDNA (SEQ ID NO: 47131), (iii) SSTAGGSSNDNA (SEQ ID NO: 47134), or (iv) SSTAGGSTNDNA (SEQ ID NO: 47137).

Embodiment B34. The modified AAV capsid protein of any one of Embodiments B1-B33, wherein peptide segment has a sequence selected from Tables 28 or 30.

Embodiment B35. The modified AAV capsid protein of any one of Embodiments B1-B34, having at least 90%, 95%, 98%, 99% or 99.5% sequence identity to the sequence of a reference AAV capsid protein.

Embodiment B36. The modified AAV capsid protein of Embodiment B35, wherein the modified AAV capsid protein has one or more amino acid insertions, deletions, substitutions, or combinations thereof as compared to the reference AAV capsid.

Embodiment B37. The modified AAV capsid protein of Embodiment B35 or B36, wherein the reference AAV capsid protein is selected from VP1, VP2 and VP3.

Embodiment B38. The modified AAV capsid protein of any one of Embodiments B35-B37, wherein the reference AAV capsid protein is a capsid protein of an AAV variant selected from the group consisting of: AAV9; AAV1; AAV2; AAV6; AAV3; AAV LK03; AAV7; AAV8; AAV hu.37; AAV rh.10; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu.9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L65; Anc80L33; Anc80L36; Anc80L44; Anc80L1; Anc80-55; Anc80-129; Anc80-156; Anc80-751; Anc80-1029; Anc80-1712; Anc110; and Anc80DI.

Embodiment B39. The modified AAV capsid protein of any one of Embodiments B1-B38, wherein the modified AAV capsid protein is an AAV capsid protein having a sequence selected from SEQ ID Nos: 54-152 and 215-220, or a fragment thereof.

Embodiment B40. The modified AAV capsid protein of any one of Embodiments B1-B39, wherein variable region I (VR I) corresponds to amino acid residues between position 259 to position 275 in SEQ ID NOs 54-152 or 215-220.

Embodiment B41. The modified AAV capsid protein of any one of Embodiments B1-B37, wherein the peptide segment is at a position corresponding to between S261 and Y274 of an AAV9 capsid protein (SEQ ID NO: 61).

Embodiment B42. The modified AAV capsid protein of any one of Embodiments B1-B37, wherein the peptide segment is at a position corresponding to between S260 and Y273 of an Anc80 capsid protein (SEQ ID NO: 132).

Embodiment B43. The modified AAV capsid protein of any one of Embodiments B1-B40, wherein the peptide segment is at a position corresponding to between S260 and Y273 of an Anc80L65 capsid protein (SEQ ID NO: 142).

Embodiment B44. The modified AAV capsid protein of any one of Embodiments B1-B40, wherein the peptide segment is at a position corresponding to between S260 and Y273 of an AAV2 capsid protein (SEQ ID NO: 55).

Embodiment B45. A polynucleotide encoding the modified AAV capsid protein of any one of Embodiments B1-B44.

Embodiment B46. A vector comprising the polynucleotide of Embodiment B45.

Embodiment B47. The vector of Embodiment B46, further comprising a promoter operably linked to the polynucleotide.

Embodiment B48. A host cell comprising the modified AAV capsid protein of any one of Embodiments B1-44, the polynucleotide specified in claim 45, or the vector of Embodiment B46 or B47.

Embodiment B49. A recombinant AAV virion (rAAV) comprising the modified AAV capsid protein of any one of Embodiments B1-B44.

Embodiment B50. The rAAV virion of Embodiment B49, further comprising at least one exogenous polynucleotide.

Embodiment B51. The rAAV virion of Embodiment B50, wherein the exogenous polynucleotide comprises a template for homology directed repair.

Embodiment B52. The rAAV virion of Embodiment B50, wherein the exogenous polynucleotide comprises at least one expressible polynucleotide encoding a therapeutic tRNA, miRNA, gene editing guide RNA, or RNA-editing guide RNA.

Embodiment B53. The rAAV virion of Embodiment B50, wherein the exogenous polynucleotide comprises an expressible polynucleotide encoding a therapeutic protein.

Embodiment B54. The rAAV virion of Embodiment B53, wherein the therapeutic protein is MTM1 or a fragment thereof.

Embodiment B55. The rAAV virion of Embodiment B54, wherein the expressible polynucleotide comprises the sequence of SEQ ID NO: 165 or a fragment thereof.

Embodiment B56. The rAAV virion of Embodiment B55, wherein the expressible polynucleotide comprises a sequence having at least 80%, 85%, 90%, 95%, 98%, 99% or 100% sequence identity to any of SEQ ID Nos: 166-170.

Embodiment B57. The rAAV virion of Embodiment B50, wherein the exogenous polynucleotide further comprises a regulatory sequence.

Embodiment B58. The rAAV virion of Embodiment B55, wherein the regulatory sequence comprises expression regulatory elements (EREs).

Embodiment B59. The rAAV virion of Embodiment B58, wherein the EREs comprise a CAG promoter.

Embodiment B60. The rAAV virion of Embodiment B58, wherein the EREs comprise a sequence having at least 90%, at least 95%, at least 98%, at least 99% or 100% sequence identity to any one of SEQ IDs NO: 171-173.

Embodiment B61. A pharmaceutical composition comprising the rAAV virion of any one of Embodiments B49-B60 a pharmaceutically acceptable carrier.

Embodiment B62. The pharmaceutical composition of Embodiment B61 which is in the form of a unit dose.

Embodiment B63. The pharmaceutical composition of Embodiment B61 or B62 which comprises 1×10¹⁰ to 1×10¹⁶ genome copy numbers (GC) of the rAAV and/or in which the rAAV concentration is 1×10¹⁰ vg/ml to 1×10¹⁶ vg/ml.

Embodiment B64. The pharmaceutical composition of any one of Embodiments B61-B63 which is formulated for parenteral administration, for example systemic (e.g., intravenous), intramuscular or subcutaneous administration.

Embodiment B65. An AAV virion comprising the modified AAV capsid protein of any one of Embodiments B1-B44 or the AAV virion of any one of Embodiments B49-B60 for use in treating and/or preventing a muscular disease and/or in muscle regeneration.

Embodiment B66. A pharmaceutical composition comprising the modified AAV capsid protein of any one of Embodiments B1-B44, and/or the AAV virion specified in any one of Embodiments B49-B60 for use in treating and/or preventing a muscular disease and/or in muscle regeneration.

Embodiment B67. A method for treating or ameliorating or preventing a disease or condition in a subject, comprising administering a therapeutically effective amount of the AAV virion of any one of Embodiments B49-B60 or the pharmaceutical composition of any one of Embodiments B61-B64.

Embodiment B68. The method of treating or ameliorating or preventing a disease according to Embodiment B67, wherein the effective dose comprises 1×10¹⁰ to 1×10¹⁶ genome copy numbers (GC) of the rAAV.

Embodiment B69. The method of Embodiment B67 or B68, wherein the administration is parenteral.

Embodiment B70. The method of Embodiment B67 or B68, wherein the administration is systemic (e.g., intravenous).

Embodiment B71. The method of Embodiment B67 or B68, wherein the administration is intramuscular.

Embodiment B72. The method of Embodiment B67 or B68, wherein the administration is subcutaneous.

Embodiment B73. The method of treating or ameliorating or preventing a disease according to Embodiment B67, wherein the disease is a muscular disease and/or the condition is muscle degeneration.

Embodiment B74. The method of treating or ameliorating or preventing a disease according to Embodiment B67, wherein said muscle is a striated muscle, preferably heart or a skeletal muscle or diaphragm.

Embodiment B75. The method of treating or ameliorating or preventing a disease according to Embodiment B67, wherein said muscular disease is a muscular dystrophy, a cardiomyopathy, a myotonia, a muscular atrophy, a myoclonus dystonia, a mitochondrial myopathy, a rhabdomyolysis, a fibromyalgia, and/or a myofascial pain syndrome.

Embodiment B76. A method of transferring an exogenous polynucleotide into a muscle cell, comprising the step of administering the AAV virion specified in any one of Embodiments B49-B60 to a subject.

Embodiment B77. The method of Embodiment B76, wherein the administration results in transfer of the exogenous polynucleotide in the muscle cell, at a muscle:liver infection ratio of greater than 1 when measured by genome copies of the AAV virion.

Embodiment B78. The method of Embodiment B76, wherein the muscle:liver infection ratio ranges from 1 to 100.

Embodiment B79. The method of Embodiment B77, wherein the muscle:liver infection ration ranges from 1 to 10.

Embodiment B80. The method of Embodiment B79, wherein the muscle:liver infection ratio ranges from 2 to 8.

Embodiment B81. The method of any one of Embodiments B76-B80, wherein the administration results in expression of the exogenous polynucleotide in the muscle cell, at a muscle:liver expression ratio of greater than 10.

Embodiment B82. The method of Embodiment B81, wherein the muscle:liver expression ratio ranges from 10 to 100.

Embodiment B83. The method of Embodiment B82, wherein the muscle:liver expression ratio ranges from 20 to 80.

Embodiment B84. The method of any one of Embodiments B76-B83, wherein the muscle:liver expression ratio ranges from 50 to 80 when measured by mRNA transcript expression.

Embodiment B85. The method of any one of Embodiments B76-B84, wherein the muscle:liver expression ratio ranges from 10 to 50 when measured by protein expression.

Embodiment B86. The method of any one of Embodiments B76-B85, wherein the muscle cell is selected from triceps surae, biceps, heart and quadricep.

Embodiment B87. Use of the AAV capsid polypeptide of any one of Embodiments B1-B44, and/or the AAV virion specified in any one of Embodiments B49-B60, for transferring an exogenous polynucleotide into a muscle cell.

Embodiment B88. The use according to Embodiment B87, wherein said use is a non-therapeutic use, preferably wherein said use is an in vitro use.

Embodiment B89. The use according to Embodiment B87, wherein the muscle cell is selected from triceps surae, biceps, heart and quadricep.

### 8. EXAMPLES

### 8.1. Example 1: Analysis of Muscle Tropism and Liver Tropism for AAV^{-Mut1-deco1}

Experiment AFT-MR0001 was designed to test the hypothesis that the peptide RGDLLLS (SEQ ID NO:1), when inserted into VR VIII to create a modified adeno-associated virus capsid protein, enhances gene delivery to skeletal muscle versus the unmodified protein. Further, the experiment was designed to test the hypothesis that the liver toggle mutation provides a structure that can determine efficiency of liver gene delivery and that the peptide insertion into VR VIII can act independently and/or synergistically. Two doses were also used, a low dose of 1×10¹³ gc/kg and a high dose of 5×10¹³ gc/kg, seeking to demonstrate possible equivalence of eGFP expression afforded by the modified AAV vector at a low dose with the eGFP expression observed with the unmodified vector at high dose. This experiment proved both hypotheses: the targeting peptide insertion into VR VIII of an adeno-associated virus VP1 protein enhances gene delivery to the muscle, and that combination with the liver-detargeting phenotype produces a vector with robust gene delivery to the muscle with the expected reduction in liver. Further, at a 5× lower dose, the level of expression between high dose unenhanced variants and matching low dose enhanced variants is not statistically significant.

### 8.2. Example 2: AAV Variants

The polynucleotide encoding the wild-type AAV9 VP1 capsid protein (SEQ ID NO: 61) or AAV^{mut1} capsid protein (SEQ ID NO: 163) was modified by inserting a coding sequence for the 7-mer peptide RGDLLLS (SEQ ID NO: 1) at a position that effected insertion of the peptide between amino acid residues 588 and 589 of the encoded polypeptide. The produced modified polynucleotides encode modified VP1 proteins referred to as: AAV^{deco1} capsid protein (SEQ ID NO: 158) or AAV^{mut1_deco1} capsid protein (SEQ ID NO: 159).

Corresponding AAV vectors were manufactured with the modified capsid proteins in the Affinia Therapeutics Vector Core facility via standard triple transfection into HEK293 cells. The AAV vectors produced by the method include AAV9 CAG.GFP (CAG.GFP construct encapsulated in AAV9 capsid), AAV^{mut1_deco1} CAG.GFP (CAG.GFP construct encapsulated in a capsid comprising the AAV^{mut1} capsid protein), AAV9^{deco1} CAG.GFP (CAG.GFP construct encapsulated in a capsid comprising the AAV^{deco1} capsid protein), and AAV^{mut1_deco1} CAG.GFP (CAG.GFP construct encapsulated in a capsid comprising the AAV^{mut1_deco1} capsid protein). Successful gene transfer by these vectors was detected by GFP expression in target cells. Please note that the vectors comprising a particular modified capsid protein is referred to in the Figures related to the following examples by the abbreviated term for the capsid protein itself, as will be clear from the context of the experiment.

### 8.3. Example 3: Confirmed Enhanced Muscle Tropism with Limited Liver Tropism for AAV^{mut1_deco1} in C57BL/6 Mice at Both Low and High Dosage Regimes

Gene transfer efficacy of each AAV vector prepared according to Example 1 was tested with three C57BL/6 mice, injected with one of the AAVs at one of the two different doses by intravenous tail vein injection. Total twenty-four mice were injected in total as summarized in the below table. The low dose was 1×10¹³ gc/kg (total 2×10¹¹ gc), and the high dose was 5×10¹³ gc/kg (total 1×10¹² gc). Additionally, a control mouse was injected with vehicle (1×PBS, 35 mM NaCl, 0.001% Pluronic) alone. Thus, a total 25 mice comprised the study.

TABLE 2

| | low dose | high dose |
|---|---|---|
| Vector (AAV) | (1 × 10¹³ gc/kg) | (5 × 10¹³ gc/kg) |
| AAV9 CAG.GFP | 3 mice | 3 mice |
| “AAV9” | | |
| AAVmut1 CAG.GFP | 3 mice | 3 mice |
| “AAV^{mut1}” | | |
| AAV9deco1 CAG.GFP | 3 mice | 3 mice |
| “AAV^{deco1}” | | |
| AAVmut1_deco1 CAG.GFP | 3 mice | 2 mice |
| “AAV^{mut1}^{_{—}}^{deco1}” | | |

| | |
|---|---|
| Control (vehicle) | 2 mice |

The mice were sacrificed 28 days after the injection. Individual tissues, notably the liver, major skeletal muscles of the hind limb, heart, and diaphragm, were collected at the time of necropsy. Tissue were immediately placed into the preservative RNAlater, after which the RNAlater was removed and the tissue flash frozen. The same tissues were fixed and embedded for sectioning and anti-GFP staining by immunohistochemistry (IHC).

GFP expression was assessed by anti-GFP IHC, and ddRT-PCR for the eGFP vector genome copies per DPG (DNA) and transcript (mRNA). The eGFR transcript level was compared against the transcript of a housekeeping standard RPP30. THC was performed at Histoserv Inc. (Germantown, MD). ddRT-PCR was performed at Affinia Therapeutics (Waltham, MA).

Images of exemplary liver and skeletal muscle tissue cross-sections obtained from the anti-GFP IHC are provided in FIGS. 4A-4J. The tissue cross-sections were stained with an anti-GFP primary antibody followed by an HRP-linked secondary staining and substrate addition. Brown staining of cells above the counterstain for intact cells and nuclei indicates eGFP expression. The vehicle control tissues from liver or skeletal muscle show the structure and organization expected from healthy tissues. AAV9 at 5×10¹³ gc/kg robustly transduces the liver and muscle cells (brown individual cells). GFP expression within the liver is reduced in mice injected with AAV-mut1-deco1, such that isolated individual cells are stained. On the other hand, GFP expression within muscle tissue was significantly increased in the mice injected with AAV-mut1-deco1.

Transgene transfer and expression capabilities of administered AAVs were also evaluated with ddPCR, by measuring amounts of DNA and mRNA of the transgene (eGFP) in the various tissue samples 28 days after injection. DNA genome copies and mRNA transcript copies of the transgene (eGFP) were quantified in comparison to the amounts of DNA genome copies or mRNA transcript copies of a house keeping gene (RPP30), respectively. Specifically, DNA genome copies are reported as vector genomes copies per diploid genome (VGC/DG). The formula for calculating the output is VGC/DG=(eGFP cp/μL÷RPP30 cp/μL)×2. RNA transcript copies are reported as % eGFP expression, which is calculated according to the formula. % eGFP expression=(eGFP cp/μL÷RPP30 cp/μL)×100.

Tissues were homogenized in a Qiagen Tissuelyser II (20 rps for 2 min) in lysis buffer from the Qiagen Dneasy Blood and Tissue Kit or the Qiagen RNeasy Lipid Tissue Mini Kit following the standard Qiagen protocol. Samples were eluted in 50 μL of buffer. Prior to analysis, DNA and RNA concentration and quality were determined using a NanoDrop One, using the nucleic acid (DNA or RNA) program. DNA samples were analyzed for biodistribution of vector genomes using a duplexed ddPCR method targeting the transgene (eGFP) and a reference gene (RPP30). RNA samples were analyzed for expression of the eGFP transgene using a duplexed, one-step RT-ddPCR method and a reference gene (RPP30).

mRNA was extracted from 30 mg sections of liver, and quadriceps. The results of the ddPCR assays are shown in FIG. 5A and FIG. 5B which show that AAV^{Mut1} reduces liver tropism but does not enhance muscle tropism, AAV^{Deco1} has high liver tropism and comparatively high muscle tropism, and that AAV^{mut1_deco1} has decreased liver tropism and increased muscle tropism compared to AAV9 (WT). Other muscle tissues examined, discussed below, and showed a similar trend and the DNA and RNA results generally agree.

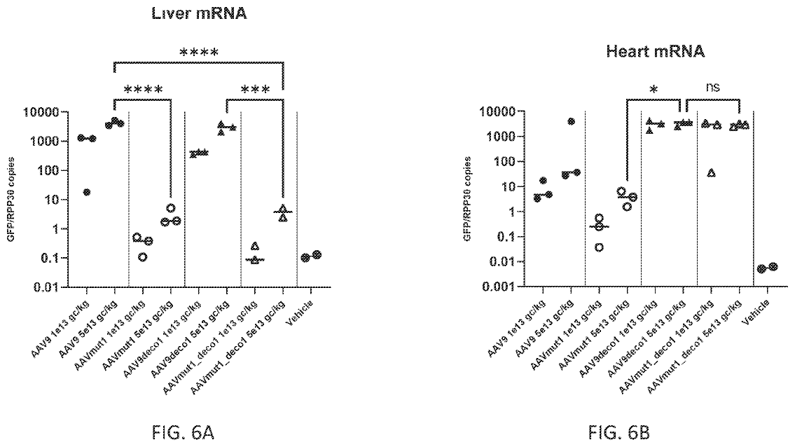

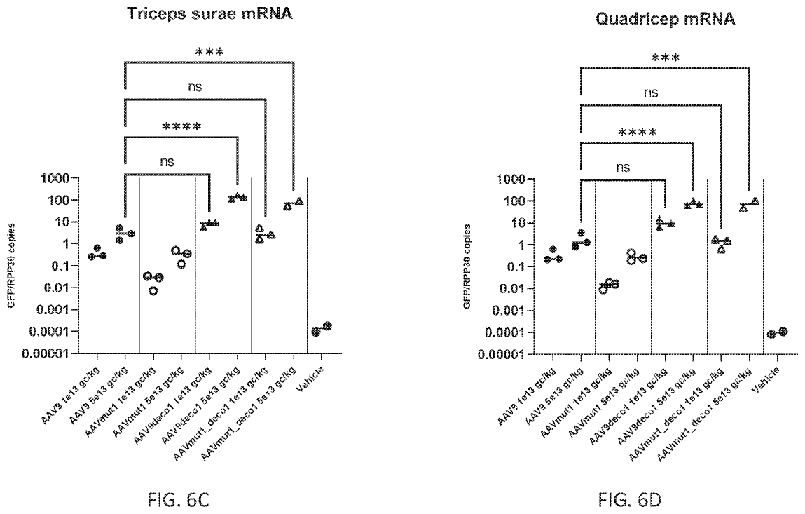

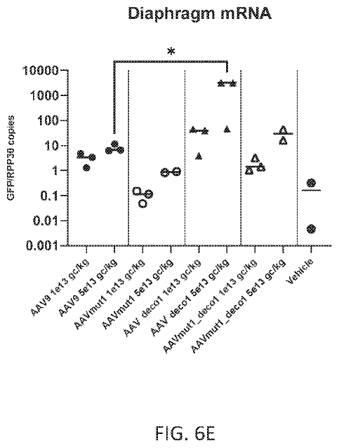

eGFP mRNA expression in various tissues was measured by RT-ddPCR and presented as the ratio of eGFP transcripts over RPP30 transcripts, a rough indicator of eGFP mRNA copies per cell. The results are provided in FIG. 6A (liver), FIG. 6B (heart), FIG. 6C (tricep surae), FIG. 6D (quadricep), and FIG. 6E (diaphragm). For each tissue, results from three biological replicates are provided for each AAV variant at each dose (high or low dose). Statistically significant differences were determined by an ANOVA 1-way test with P-values and indicated with asterisks. * P<0.1, ** P<0.01, *** P<0.001. **** P<0.0001, ns=not significant.

FIG. 6A provides the ratio of eGFP to RPP30 transcripts in the liver. At 5×10¹³ gc/kg dose, both AAV^{mut1} and AAV^{mut1_deco1} had a greater than 3-log lower levels of eGFF expression in the liver compared to AAV9. AAV^{deco1} had almost 3-logs higher expression in the liver than AAV^{mut1_deco1}.

FIG. 6B shows the ratio of eGFP to RPP30 transcripts in the heart. Both AAV^{deco1} and AAV^{mut1} deco1 had higher expression in the heart, although the difference and significance are reduced by a single outlier within the AAV9 at 5×10¹³ gc/kg dose group, and possible signal saturation within the AAV^{deco} and AAV^{mut1_deco1} high dose group. The level of expression is significantly higher in AAV^{deco1} compared to AAVmut1 at 5×10¹³ gc/kg, and there was no significant difference between high dose AAV^{deco1} and AAV^{mut1_deco1}, notwithstanding the possible signal saturation.

FIG. 6C shows the ratio of eGFP to RPP30 transcripts in the triceps surae. Within the 5×10¹³ gc/kg groups, there was more than 1-log increase in the eGFP per RPP30 mRNA ratio in AAV9^{deco1} and AAV^{mut1_deco1} compared to AAV9 in the calf muscle tissue of the study subjects. Importantly, there was no significant difference between the high dose AAV9 group and the 1×10¹³ gc/kg low dose groups of AAV^{deco1} and AAV^{mut1_deco1}.

FIG. 6D shows the ratio of eGFP to RPP30 transcripts in the quadricep. Results were similar to the triceps surae, the other skeletal muscle tested in this study. Within the 5×10¹³ gc/kg groups, there is more than 1 log increase in eGFP per RPP30 mRNA ratio in AAV^{deco1} and AAV^{mut1_deco1} compared to AAV9 in quadricep tissue of the study subjects. Importantly, there is no significant difference between the high dose AAV9 group and the 1×10¹³ gc/kg low dose groups of AAV^{deco1} and AAV^{mut1_deco1}.

FIG. 6E shows the ratio of eGFP to RPP30 transcripts in the diaphragm.

Increase of gene delivery efficacy in deco-containing vectors was also observed in the diaphragm, but in this study all but one comparison exceeded the threshold of significance: high dose AAV9 versus high dose AAV^{deco}.

### 8.4. Example 4: Enhanced Muscle Tropism with Limited Liver Tropism for AAV^{-mut1-deco1} Confirmed at the Earlier d14 Time Point in C57BL/6 Mice

Gene transfer efficacy of AAV9 vector and AAV^{mut1_deco1} vector was tested with groups of three C57BL/6 mice, injected with one of the AAVs by intravenous tail vein injection. Total thirteen mice were injected in total as summarized in the below table. The dose was 1×10¹³ gc/kg (total 2×10¹¹ gc). Additionally, a control mouse was injected with vehicle (1×PBS, 35 mM NaCl, 0.001% pluronic) alone.

TABLE 3

| | | Dose | No. of | Study | |
|---|---|---|---|---|---|
| Treatment | Route | (vgs) | Animal | Duration | Necroscopy |
| Vehicle /control | IV | 0 | 1 | 14 days | Organ |
| AAV^{mut1} | IV | 1.00E + 13 | 3 | | collection |
| AAV^{mut1}_deco1 | IV | 1.00E + 13 | 3 | | |
| AAV^{mut1} | IV | 1.00E + 13 | 3 | 28 days | |
| AAV^{mut1}_deco1 | IV | 1.00E + 13 | 3 | | |

The mice were sacrificed 14 or 28 days after the injection. Individual tissues, notably the liver and major skeletal muscles of the hind limb (quad), were collected at the time of necropsy. Tissues were immediately placed into the preservative RNAlater, after which the RNAlater was removed and the tissue flash frozen. The same tissues were fixed and embedded for sectioning and anti-GFP staining by immunohistochemistry (IHC).

eGFP expression was assessed by anti-GFP IHC. IHC was performed at Histoserv Inc. (Germantown, MD). ddRT-PCR was performed at Affinia Therapeutics (Waltham, MA) as described above.

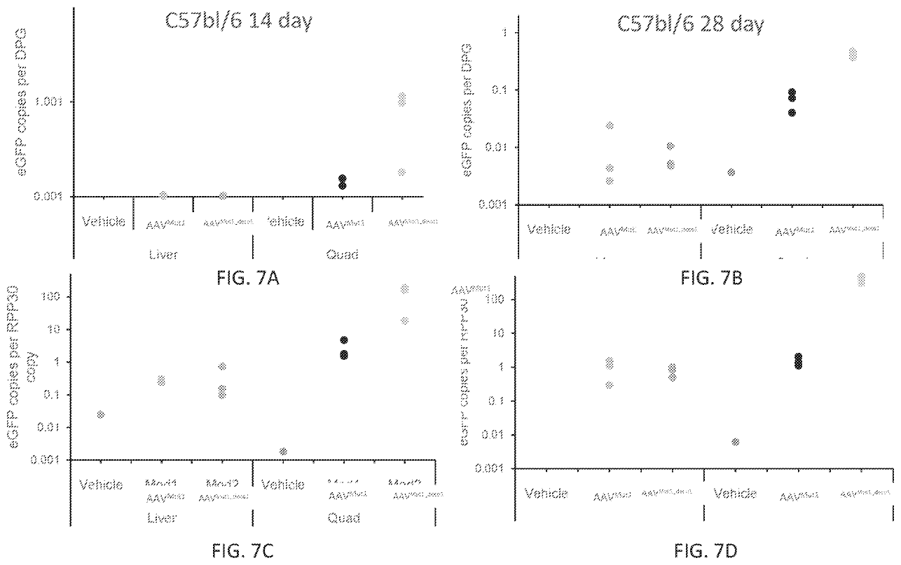

DNA and RNA were extracted from 30 mg sections. DNA and RNA samples were assayed for eGFP vector genome or mRNA transcript by ddRT-PCR and normalized to murine RPP30 genomic copies or RPP30 mRNA copies, respectively. Triplicate technical replicates were performed. The results are shown in FIGS. 7A-7D.

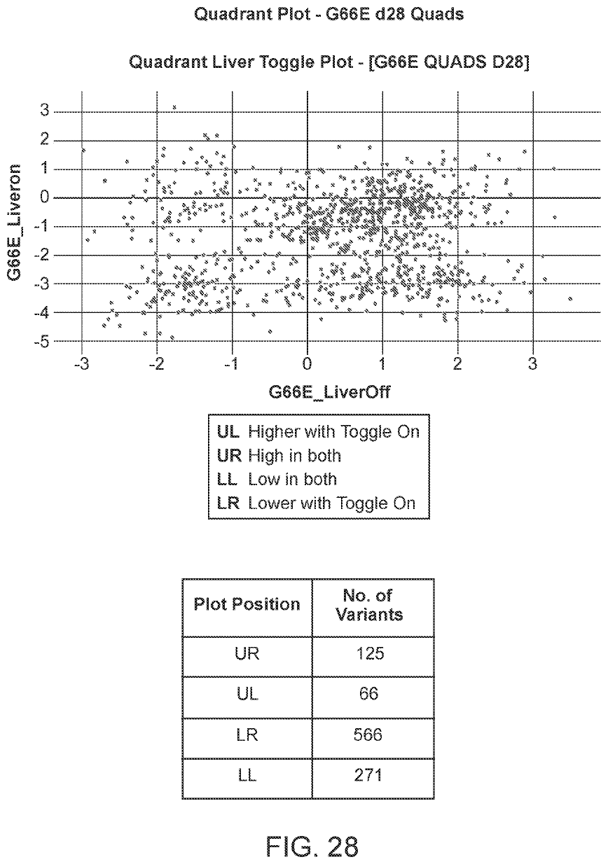

FIGS. 7A-7B show eGFP vector genome (DNA) in liver and quad tissues of C57BL/6 mice 14 days (FIG. 7A) or 28 days (FIG. 7B) after treatment with vehicle. AAV^{Mut1} and AAV^{Mut1_deco1} AAV vectors. FIGS. 7C-7D show eGFP mRNA expression in liver and quad tissues of C57BL/6 mice 14 days (FIG. 7C) or 28 days (FIG. 7D) after treatment with vehicle, AAV^{Mut1} and AAV^{Mut1_deco1} AAV vectors.

As can be seen from these data, AAV^{Mut1_deco1} enhancement of muscle tropism is observable at d14, AAV^{Mut1} and AAV^{Mut1_deco1} vector genome copies (VGs) are stable from d14 to d28; AAV^{Mut1_deco1} enhancement leads to greater accumulation of eGFP signal; and liver tropism is consistently low through all samples.

### 8.5. Example 5: Confirmed Enhanced Muscle Tropism with Limited Tropism to Liver and Other Organs for AAV^{mut1_deco1} in BALB/c Mice

Gene transfer efficacy of AAV^{mut1} and AAV^{mut1_deco1} were tested with three or six BALB/c mice, injected with one of the vectors at 5×10¹³ gc/kg (total 1×10¹² gc) by intravenous tail vein injection. Additionally, control mice were injected with vehicle (1×PBS, 35 mM NaCl, 0.001% pluronic) alone. Total twelve mice were injected in total as summarized in the below table.

TABLE 4

| | 5 × 10¹³ gc/kg |
|---|---|
| Vector (AAV) | (total 1 × 10¹² gc) |
| Control (vehicle) | 3 mice |
| AAVmut1 CAG.GFP | 6 mice |
| AAVmut1_deco1 CAG.GFP | 3 mice |

The mice were sacrificed 28 days after the injection. Individual tissues, notably the liver, major skeletal muscles of the hind limb, heart, diaphragm, brain, spinal cord, and spleen were collected at the time of necropsy.

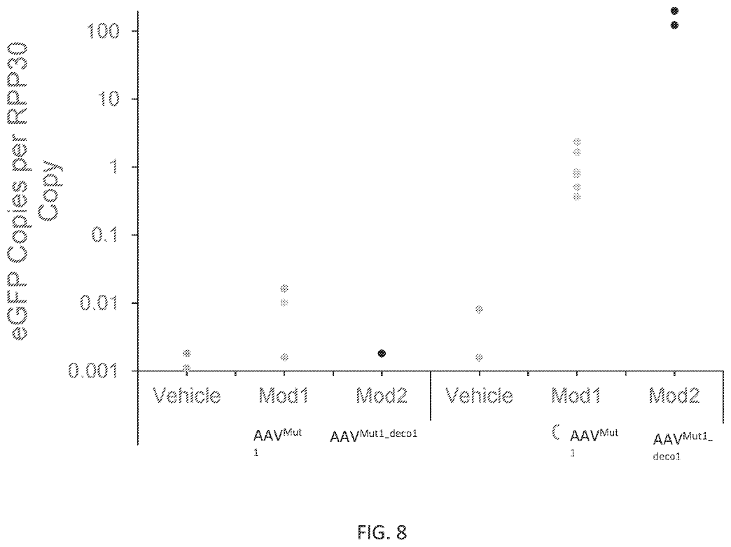

DNA and RNA were extracted from 30 mg sections of liver and quadricep. DNA and RNA samples were assayed for eGFP vector genome or mRNA by ddRT-PCR and normalized to murine RPP30 genomic copies or RPP30 mRNA copies. Triplicate technical replicates were performed. The results are provided in FIG. 8.

The results show no increase in liver tropism with AAV^{mut1 deco1} but increase of tropism in the quadriceps compared to AAV^{mut1}. Further the data showed a similar AAV^{mut1 deco1} enhancement in the heart, triceps surae, and diaphragm compared to AAV^{mut1} There was no significant difference found in the spleen, spinal cord, or liver.

### 8.6. Example 6: Summary of Mouse Data

The below Table 5 exemplifies the Muscle:Liver infection ratios calculated for the DNA biodistribution data, the RNA expression data and the IHC expression data obtained for administration of AAV^{mut1_deco1} vector compared to AAV9 vector in Mice.

TABLE 5

| | | | DNA | | RNA | | IHC |
|---|---|---|---|---|---|---|---|
| | | DNA | Muscle:Liver | | Muscle:Liver | | Muscle:Liver |
| | | Biodis- | infection | RNA | infection | IHC | infection |
| Tissue | Treatment | tribution† | ratio | % Expression† | ratio | Expression† | ratio |
| Liver | AAV9 | 151.4 | | 418083 | | 99 | |
| Liver | AAV^{mut1}^{_{—}}^{deco1} | 0* | | 373.9 | | 4.3 | |
| Quadriceps | AAV9 | 0.9 | 0.01 | 184.0 | 0.00 | 0.7 | 0.01 |
| Quadriceps | AAV^{mut1}^{_{—}}^{deco1} | 0.3 | >10,000* | 7277.0 | 19.46 | 8.7 | 2.02 |
| Triceps | AAV9 | 0.3 | 0.00 | 322.2 | 0.00 | 0.0 | 0.00 |
| Surae | | | | | | | |
| Triceps | AAV^{mut1}^{_{—}}^{deco1} | 0.5 | >10,000* | 7092.3 | 18.97 | 15.0 | 3.49 |
| Surae | | | | | | | |
| Heart | AAV9 | 0.5 | 0.00 | 4975.2 | 0.01 | 10.7 | 0.11 |
| Heart | AAV^{mut1}^{_{—}}^{deco1} | 3.8 | >10,000* | 322.2 | 0.86 | 68.3 | 15.88 |

*when liver value is zero, then the ratio is >10,000 by convention.

### 8.7. Example 7: Confirmed Enhanced AAV^{mut1_deco1} Muscle Tropism with Limited Liver Tropism Confirmed in NHP

The objective of this study is to confirm liver retargeting and muscle transduction superiority of AAV^{mut1_deco1} vector compared to AAV9 vector in non-human primates (NHP) as was observed in mice. The results confirm enhanced muscle transduction superiority and liver de-targeting of AAV^{Mut1_Deco1} vector compared to AAV9.

Two AAV constructs were used in the experiment: (i) AAV^{-mut1.deco1}-CAG-GFP, and (ii) AAV9-CAG-GFP, each including an AAV genome construct containing a coding sequence of GFP. GFP was used to detect distribution of AAVs and expression of the transgene. Marmoset monkeys were used as the subject animals.

Total of 7 animals were divided into 3 groups as summarized in the below TABLE #. Immunosuppression of the animals began 7 days prior to AAV administration. Group 1 is a control animal administered with vehicle. Animals in Group 2 and 3 were administered with 1×10¹⁴ vg (viral genome or GC) of AAV9 vector or AAV^{mut1_deco1} vector by IV to the right saphenous vein. Animals were sacrificed on day 28 after the vehicle or AAV vector administration and their organ samples were collected for analysis.

TABLE 6

| | Route of | | | | |
|---|---|---|---|---|---|
| | Adminis- | Dose | No. of | Volume | Study |
| Treatment | tration | (vgs) | Animal | (ml) | Duration |
| Vehicle/ | IV | 0 | 1 | 0.625 | 28 days |
| control | | | | | |
| AAV9 (WT) | IV | 1.00E+14 | 3 | 0.625 | |
| AAV^{mut1}^{_{—}}^{deco1} | IV | 1.00E+14 | 3 | 0.625 | |

IHC for GFP expression were scored (blinded) by a pathologist. A second pathologist peer reviewed the data. Initial assessment for GFP expression by IHC was conducted on one section per tissue-referred to as Run 1 tissues and included liver, heart and skeletal muscle (right and left sides-tibialis, biceps, quadriceps, gastrocnemius. Two additional sections per muscle group were run to assess consistency of expression within each muscle group-referred to as Run 2.

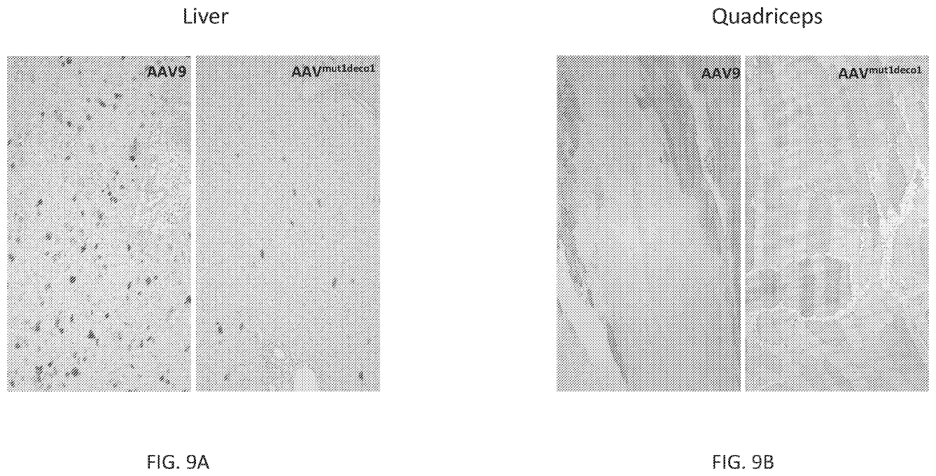

Analysis of Run 1 tissue samples is shown in FIGS. 9A and 9B. Exemplary IHC liver tissue is shown in FIG. 9A, obtained from AAV9 treated animal on the left, and AAV^{mut1_deco1} treated animal illustrated on the right side of the chart. Exemplary IHC quadriceps tissue is shown in FIG. 9B, obtained from AAV9 treated animal on the left, and AAV^{mut1_deco1} treated animal on the right.

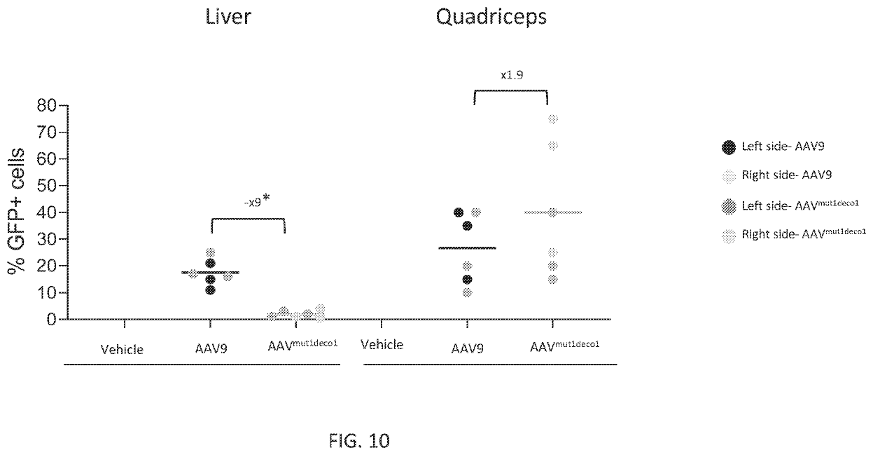

FIG. 10 shows the % GFP positive cells in the liver tissue (right and left side of the organ) and quadriceps tissue (right and left leg) in slides obtained from Run 1 for each animal administered vehicle or AAV (AAV9 or AAV^{mut1_deco1}).

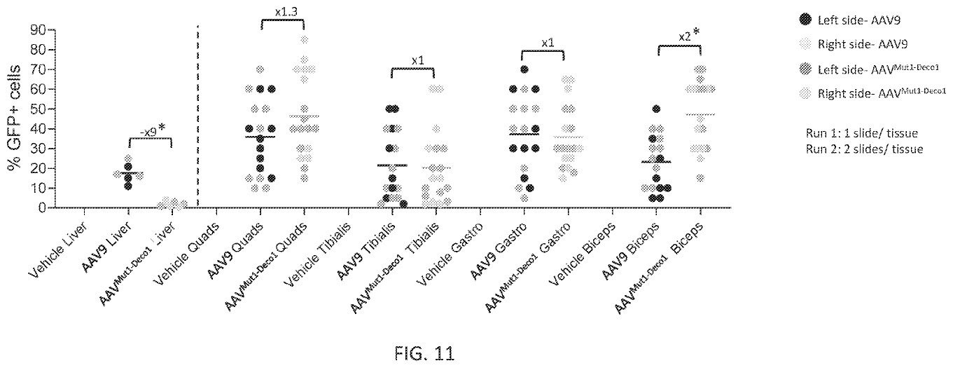

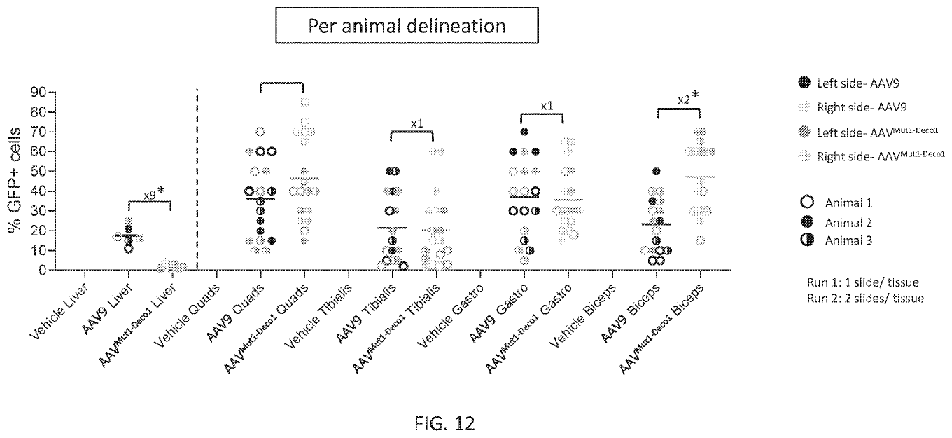

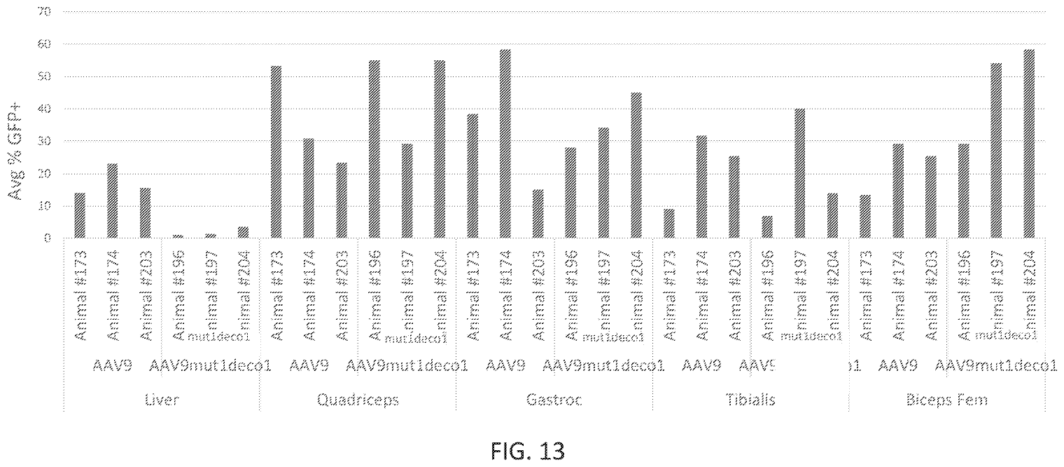

FIG. 11 shows the % GFP positive cells in various skeletal muscle and liver tissue (average from Runs 1 and 2) for each animal administered vehicle and AAV (AAV9 or AAV^{mut1_deco1}). FIG. 12 shows the % GFP positive cells per animal in various skeletal muscle and liver tissue (average from Runs 1 and 2) for each animal administered vehicle and AAV (AAV9) or AAV^{mut1_deco1}). FIG. 13 shows the average combined quantification of % GFP positive cells per animal in various skeletal muscle and liver tissue (average from Runs 1 and 2) for each animal administered vehicle and vector (AAV9 or AAV^{mut1_deco1}).

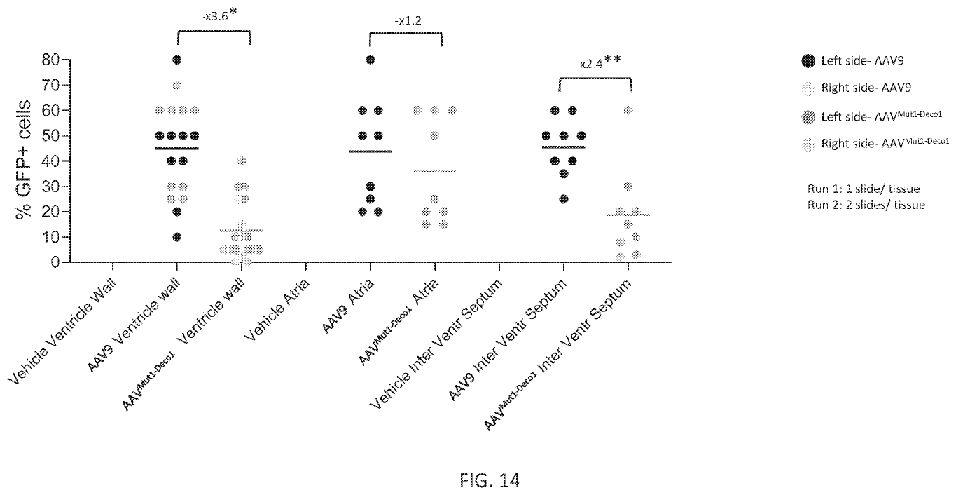

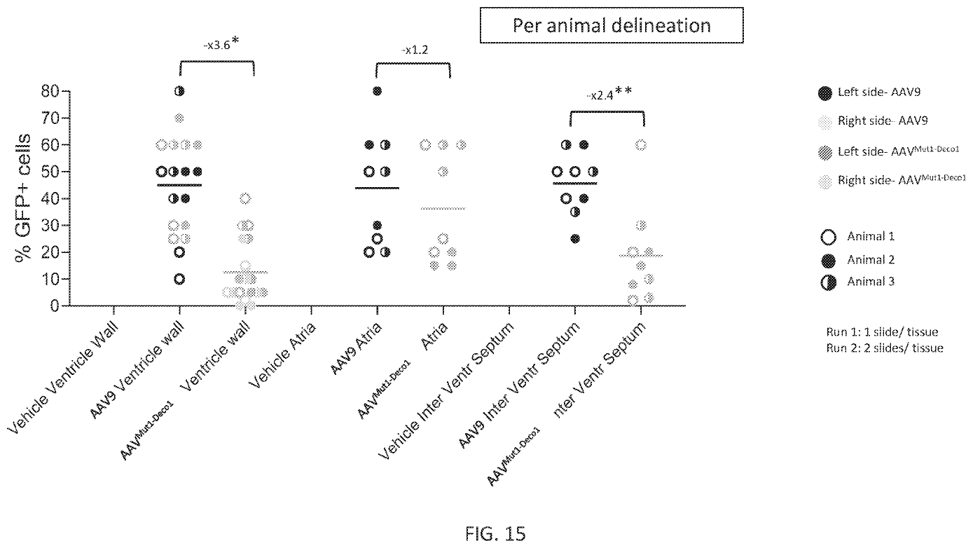

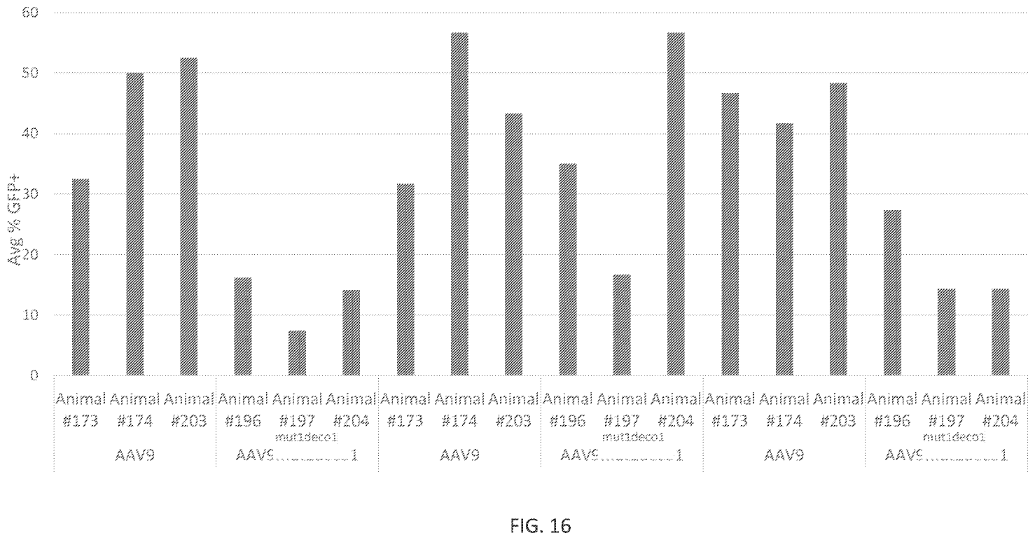

FIG. 14 shows the % GFP positive cells in various cardiac tissue (average from Runs 1 and 2) for each animal administered vehicle and AAV (AAV9 or AAV^{mut1_deco1}). FIG. 15 shows the % GFP positive cells per animal in various cardiac muscle (average from Runs 1 and 2) for each animal administered vehicle and AAV (AAV9 or AAV^{mut1_deco1}). FIG. 16 shows the average % GFP positive cells per animal in various cardiac muscle (average from Runs 1 and 2) for each animal administered vehicle and AAV (AAV9 or AAV^{mut1_deco1}).

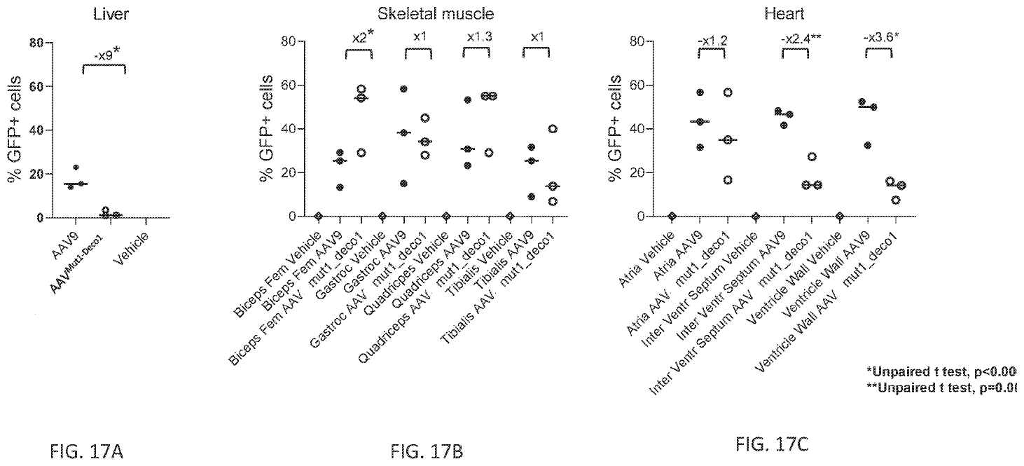

FIGS. 17A-17C show the average % GFP positive cells per animal in various tissues (average from Runs 1 and 2) for vehicle, AAV9 and AAV^{mut1_deco1}. FIG. 17A shows average % GFP positive cells per animal in liver tissue. FIG. 17B shows average % GFP positive cells per animal in various skeletal muscle tissue. FIG. 17C shows average % GFP positive cells per animal in various cardiac tissue.

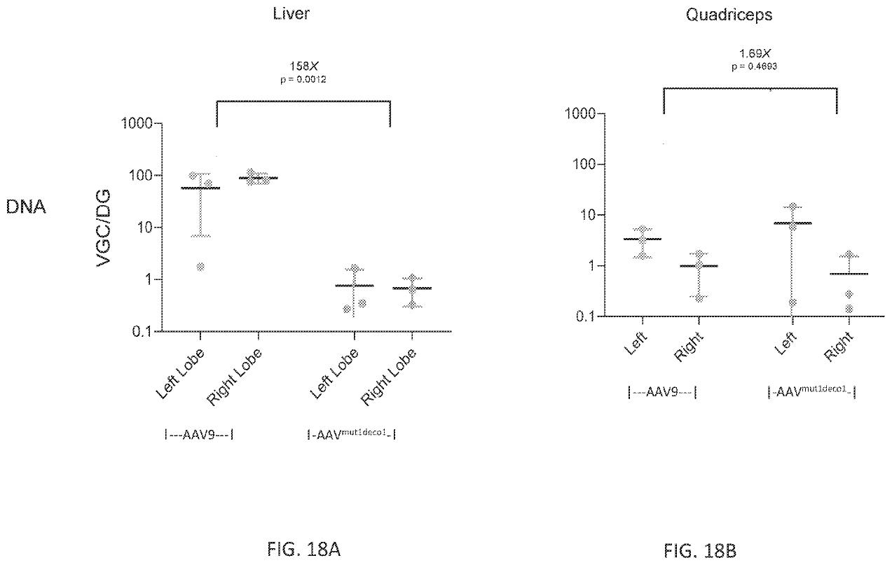

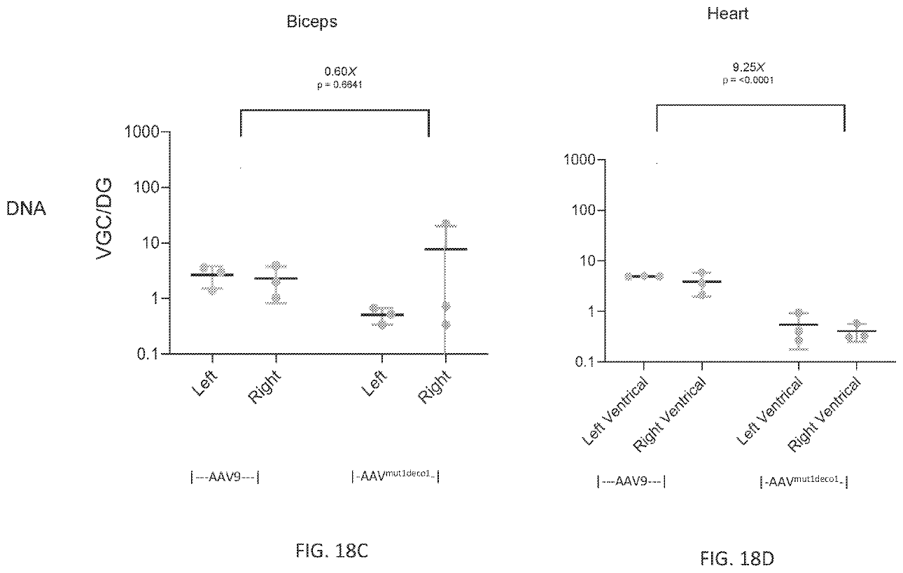

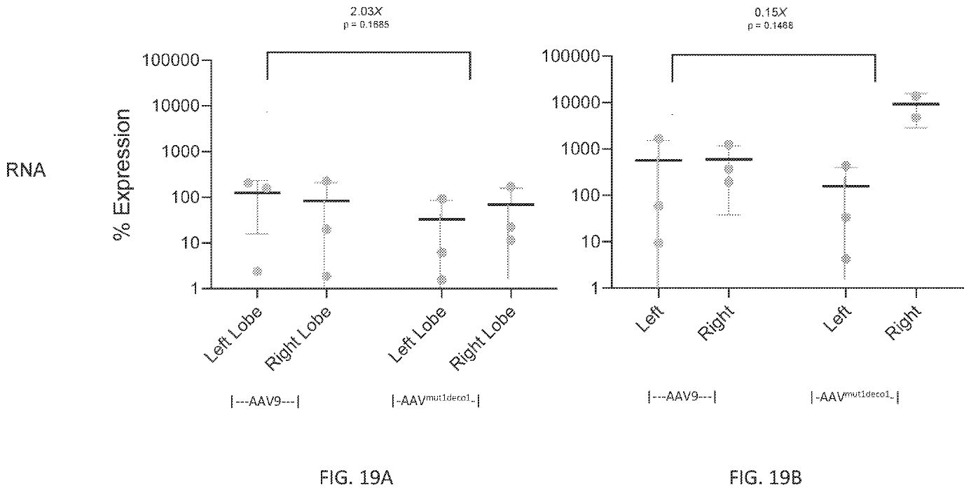

DNA samples were analyzed for biodistribution of vector genomes in the liver and quadriceps tissue using a duplexed ddPCR method targeting the transgene (eGFP) and a reference gene (RPP30). The results are shown in FIG. 18A (liver), FIG. 18B (quadriceps). FIG. 18C (biceps), and FIG. 18D (heart) where the x-axis represents AAV vectors (wild type AAV9 on the left and AAV^{mut1deco1} on the right) and whether the sample was taken from the left or right side of the organ/animal.

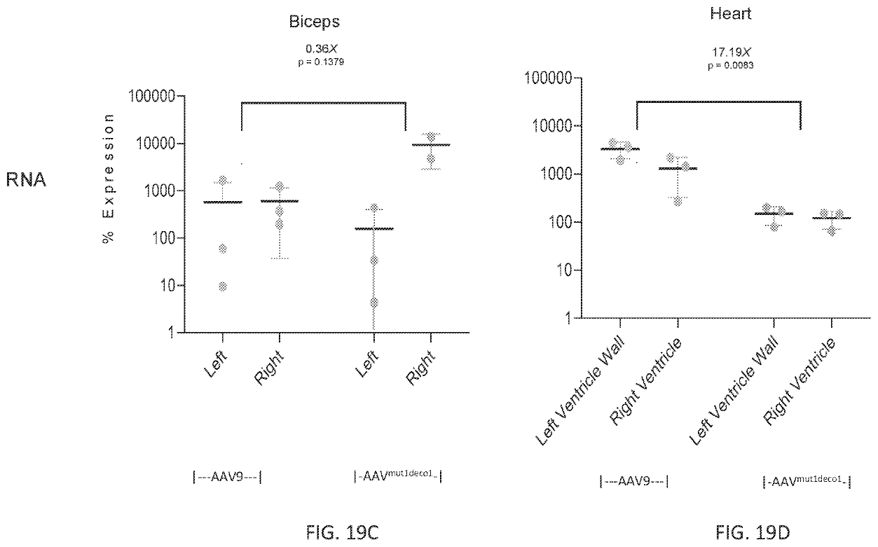

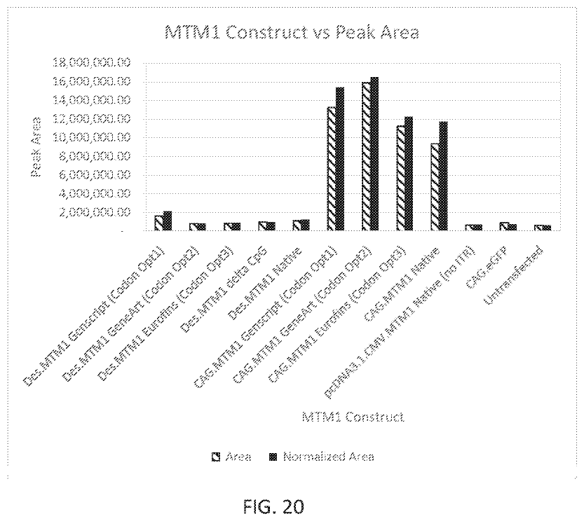

mRNA transcript amounts measured by eGFP copies of eGFP over RPP30 mRNA are shown in FIG. 19A (liver), FIG. 19B (quadriceps). FIG. 19C (biceps), and FIG. 19D (heart). The x-axis represents AAV vectors (wild type AAV9 on the left and AAV^{mut1deco1} on the right) and whether the sample was taken from the left or right side of the organ/animal.

The DNA, RNA and IHC expression data obtained from NHP experiments are quantified and summarized in the below Table 7 where each IHC stain is a technical replicate, data from all tissues combined including left and right sides; averages of the data obtained for all three animals is shown. Notably, heart data includes data from ventricles and atria but does not include septum.

TABLE 7

| | | DNA Bio- | | IHC |
|---|---|---|---|---|
| | | distribution^{†} | RNA % | Expression^{†} |
| Tissue | Treatment | VGC/DG | Expression^{†} | % GFP |
| Liver | AAV9 | 73.7 (32.7) | 104.1 (107.6) | 17.5 (4.9) |
| Liver | AAV^{mut1}^{_{—}}^{deco1} | 0.7 (0.6) | 51.1 (68.3) | 1.9 (1.4) |
| Quadriceps | AAV9 | 2.2 (1.3) | 589.5(693.1) | 35.8 (17.7) |
| Quadriceps | AAV^{mut1}^{_{—}}^{deco1} | 3.8 (3.6) | 3814.4 (5954.5) | 46.4 (21.0) |
| Biceps | AAV9 | 2.5 (1.0) | 1327.3 (3005.6) | 22.6 (11.6) |
| Biceps | AAV^{mut1}^{_{—}}^{deco1} | 4.4 (6.0) | 3677.6 (3962.5) | 47.2 (14.9) |
| Heart | AAV9 | 3.6 (2.2) | 2303.6 (1503.9) | 44.6 (10.1) |
| Heart | AAV^{mut1}^{_{—}}^{deco1} | 0.5 (1.5) | 134.3 (52.1) | 20.44 (15.8) |

^{†}Mean (St. Dev.)

The below Table & exemplifies the Muscle:Liver infection ratios calculated for the DNA biodistribution data, the RNA expression data and the IHC expression data obtained for administration of AAV^{mut1_deco1} compared to AAV9 in non-human primates (NHP) as shown in the table above.

TABLE 8

| | | | DNA | | RNA | | IHC |
|---|---|---|---|---|---|---|---|
| | | DNA | Muscle:Liver | | Muscle:Liver | | Muscle:Liver |
| | | Biodis- | infection | RNA | infection | IHC | infection |
| Tissue | Treatment | tribution† | ratio | % Expression† | ratio | Expression† | ratio |
| Liver | AAV9 | 73.7 | | 104.1 | | 17.5 | |
| Liver | AAV^{mut1}^{_{—}}^{deco1} | 0.7 | | 51.1 | | 1.9 | |
| Quadriceps | AAV9 | 2.2 | 0.03 | 589.5 | 5.66 | 35.8 | 2.05 |
| Quadriceps | AAV^{mut1}^{_{—}}^{deco1} | 3.8 | 5.43 | 3814.4 | 74.65 | 46.4 | 24.42 |
| Biceps | AAV9 | 2.5 | 0.03 | 1327.3 | 12.75 | 22.6 | 1.29 |
| Biceps | AAV^{mut1}^{_{—}}^{deco1} | 4.4 | 6.29 | 3677.6 | 71.97 | 47.2 | 24.84 |
| Heart | AAV9 | 3.6 | 0.05 | 2303.6 | 22.13 | 44.6 | 2.55 |
| Heart | AAV^{mut1}^{_{—}}^{deco1} | 0.5 | 0.71 | 134.3 | 2.63 | 20.44 | 10.76 |

### 8.8. Example 8: Development of rAAV Constructs with High MTM1 Expression

Myotubular myopathy (XLMTM, OMIM 310400) is a severe congenital muscular disease due to mutations in the myotubularin gene (MTM1) and characterized by the presence of small myofibers with frequent occurrence of central nuclei. Myotubularin is a ubiquitously expressed phosphoinositide phosphatase with a muscle-specific role in man and mouse that is poorly understood.

The objective of the current study was to identify a promoter that provides a broad biodistribution of expression within skeletal muscle. We have constructed nine human MTM1 expressing AAV gene expression constructs with various promoters and transgenes to transduce skeletal muscle and express adequate amounts of MTM1 protein for the treatment of XLMTM.

### 8.8.1. Materials & Methods

### 8.8.1.1 Cloning of MTM1 Expression Constructs

A nucleotide sequence was synthesized to include the untranslated first exon and a portion of the intron from the human Cytomegalovirus (hCMV) IE gene, a portion of the intron of the second intron of the human beta globin gene, a portion of the 3^{rd} exon of the human beta globin gene, a NotI restriction site, a predicted optimal Kozak sequence, a codon optimized human MTM1 CDS with a modified stop codon using a sequence provided by Genscript, a PacI restriction site which overlaps with the modified stop codon, the Rabbit beta-globin PolyA signal sequence, an AvrII site, and the first 10 bp of the AAV2 ITR. This fragment and SA024, an AAV2 ITR plasmid containing a Desmin promoter, a chimeric intron and exon of the CMV IE gene and human beta globin, a gene of interest, and a Rabbit beta-globin PolyA signal, were digested with BstBI, which has a site in CMV IE exon I, and XhoI, which has a site in the Rabbit beta-globin PolyA signal. Portions of SA024 containing the first ITR and expression regulatory sequences 5′ to the gene of interest are provided as SEQ ID NO:204 and the portions of SA024 from the 3′ of the open reading frame of the gene of interest through the second ITR are provided as SEQ ID NO:205. The 2443 bp long fragment containing MTM1 and the 6693 bp long fragment containing the plasmid elements were isolated by agarose gel electrophoresis and eluted from the agarose using NEB Monarch DNA Gel isolation kit. Ligations of the sticky end fragments were performed with T4 DNA ligase. Successful ligation products were isolated from E. coli transformants and confirmed by restriction digest and Sanger sequencing to confirm the insertion of the codon optimized MTM1 sequence and the additional features. The portions of the vector within the ITRs (and including the ITRs) are provided as SEQ ID NO.181.

Two additional codon optimizations for the MTM1 CDS using sequences derived from algorithms provided by GeneArt and Eurofins as well as the native MTM1 CDS (SEQ ID NO:202) were synthesized (used in SEQ ID NOS: 182, 183 and 184 described below, respectively). These three sequences with the addition of a NotI site and a Kozak site at the 5′ end and a modified stop codon and PacI site at the 3′ end of the sequence were synthesized at GeneArt, Eurofins, and Genscript, respectively, An additional sequence was synthesized by Genscript using an algorithm to both optimize the codons for expression and reduce the number of CpG sequences within the synthetic product (used in SEQ ID NO:185). These fragments or plasmids containing these fragments were digested with NotI and Pack along with the vector containing SEQ ID NO: 181. The 1823 bp fragment containing the MTM1 CDSs and the 7350 bp fragment containing the plasmid and ITR sequence and other SEQ ID NO: 181 features were isolated by agarose gel electrophoresis and eluted from the agarose using NEB Monarch DNA Gel isolation kit. Ligations of the sticky end fragments were performed with T4 DNA ligase. Successful ligation products were isolated from E. coli transformants and confirmed by restriction digest and Sanger sequencing to confirm the insertion of the codon optimized or native MTM1 sequence and the additional features (the portions of these vectors within (and including) the ITRs are provided as SEQ ID NOS: 182-185).

Constructs containing a promoter which is a hybrid of the CMV immediate early enhancer, and the chicken beta-actin promoter were also made. The hybrid is referred to as the CAG promoter. The CAG promoter was amplified from construct 7701591057 (the portion of which within (and including) the ITRs is provided as SEQ ID NO:201), which had been synthesized previously, using a 5′ primer with the sequence SEQ ID NO: 209 (ttttGGTACCgacattgattattgactagttatt) which contains a KpnI restriction site and a Poly T tag to aid in restriction digestion and a region matching the start of the CMV immediate early promoter in a linear amplification reaction. The amplification product was isolated from the amplification mixture with NEB Monarch DNA Gel isolation kit. A second amplification step was performed with a primer with the sequence SEQ ID NO: 210 (aaaaaa gatate cgccegeegegc) which contains a region matching the reverse complement of the Chicken beta actin promoter, an EcoRV restriction site, and a poly A sequence to aid in fragment digestion. The 675 base pair fragment (SEQ ID NO:200) was isolated by agarose gel electrophoresis and eluted from the agarose using NEB Monarch DNA Gel isolation kit. The 675 bp fragment and SEQ ID NO:181 vector were digested with KpnI and EcoRV. The 663 bp digested fragment of the amplification and the 8581 bp fragment of the SEQ ID NO: 181 vector were isolated by agarose gel electrophoresis and eluted from the agarose using NEB Monarch DNA Gel isolation kit. Ligations of the sticky end fragments were performed with T4 DNA ligase. Successful ligation products were isolated from E. coli transformants and confirmed by restriction digest and Sanger sequencing to confirm the insertion of the CAG promoter sequence into the vector containing SEQ ID NO: 181 resulting in a vector containing SEQ ID NO.186, which includes an MTM1 CDS with codon optimizations provided by Genscript.

To insert the GeneArt codon optimized MTM1 sequence and the Eurofins codon optimized MTM1 sequence into the SEQ ID NO: 186 containing vector, vectors with SEQ ID NO: 182. SEQ ID NO: 183, and SEQ ID NO: 186 were digested with KpnI and EcoRV. The 663 base pair fragment from the SEQ ID NO: 186 containing vector and the 8581 bp fragment from the digests of the vector with SEQ ID NO:182 and SEQ ID NO:183 were isolated by agarose gel electrophoresis and eluted from the agarose using NEB Monarch DNA Gel isolation kit. Ligations of the sticky end fragments were performed with T4 DNA ligase. Successful ligation products were isolated from E. coli transformants and confirmed by restriction digest and Sanger sequencing to confirm the insertion of the CAG promoter sequence into the SEQ ID NO. 182 and SEQ ID NO: 183 vectors, resulting in vectors containing SEQ ID NO: 187 (GeneArt) and SEQ ID NO: 188 (Eurofins).

To insert the native MTM1 sequence into the vector containing SEQ ID NO: 186, the native MTM1 sequence was amplified from the vector containing SEQ ID NO: 184 using a 5′ primer with the sequence TTTGAGCGGCCOCCA (SEQ ID NO: 215) which corresponds to the Kozak and start sequence of MTM1 and contains a NotI restriction site and a 3′ primer with the sequence GATCTTAATTAAAAGTGAGTTTGCACATGGG (SEQ ID NO: 216) which contains the reverse complement to the 3′ end of MTM1, an altered stop codon, and a PacI restriction site. The 1837 base pair PCR product (SEQ ID NO: 19) was isolated by agarose gel electrophoresis and eluted from the agarose using NEB Monarch DNA Gel isolation kit. The purified amplicon and the vector containing SEQ ID NO:186 were digested with NotI and PacI. The 1823 bp fragment containing the MTM1 CDS and the 7350 bp fragment containing the plasmid and ITR sequence and other SEQ ID NO.186 features were isolated by agarose gel electrophoresis and eluted from the agarose using NEB Monarch DNA Gel isolation kit. Ligations of the sticky end fragments were performed with T4 DNA ligase. Successful ligation products were isolated from E. coli transformants and confirmed by restriction digest and Sanger sequencing to confirm the insertion of the native MTM1 sequence and the additional features SEQ ID NO: 186 resulting in a vector with SEQ ID NO: 189.

### 8.8.1.2 Cloning of Self-Complementary MTM1 Expression Constructs

The vectors described above are single stranded vectors. To overcome potential limitations on expression from these vectors, self-complementary vectors were constructed. Genscript synthesized a vector which contained the sequence of the miniTK promoter, an alternate gene of interest, the Rabbit beta-globin Poly A signal, and an AAV2 ITR which contains a deletion in the D region of the ITR. A miniTK portion of the vector is provided as SEQ ID NO: 190 and the portion of the vector containing the rabbit globin poly A and ITR is provided as SEQ ID NO: 191. This synthetic sequence was flanked by SalI site at the 5′ end and AseI at the 3′ end. This fragment was introduced into the vector comprising SEQ ID NO:201 via restriction enzyme digestion, agarose gel fragment isolation, and T4 DNA ligase ligation. Successful ligation products were isolated from E. coli transformants and confirmed by restriction digest and Sanger sequencing to confirm the insertion of self-complementary vector sequence into the vector comprising SEQ ID NO:201 resulting in a vector containing SEQ ID NOS: 190 and 191. The first ITR and miniTK portions of the vector are provided as SEQ ID NO:206 and the rabbit poly and second ITR portions of the vector are provided as SEQ ID NO:207.

To create self-complementary vectors of the appropriate size for successful packaging into AAV capsids, the miniTK promoter was synthesized with KpnI restriction site at the 5′ end and a NotI site at the 3′ end. Additionally, bases were added to the synthesize product to enhance the efficiency of restriction digestion SEQ ID NO: 192. The fragment containing SEQ ID NO: 192 was digested with KpnI and NotI and inserted into a vector containing SEQ ID NO: 184 via the same restriction sites following agarose gel electrophoresis, gel extraction, T4 ligation. This vector (the portion of which within (and including) the ITRs is provided as SEQ ID NO: 11B) after sequencing, was determined to have an undesired deletion in the 5′ ITR. However, the insert of SEQ ID NO: 193 was identical to the desired sequence. The miniTK-native MTM1 sequence was PCR amplified using the 5′ primer SEQ ID NO. 211 (tttttGtcGACTTCGCATATTAAGGTGACGCGT) which contains a polyT sequence to aid in restriction digestion, the KpnI site, and the 5′ end of the miniTK promoter and the 3′ primer SEQ ID NO. 212 (tttttt cctagg gagTGAGAGACACAAAAAATTCCAACACAC), which contains a polyT sequence to aid in restriction digestion, an AvrII site, and the reverse complement of the 3′ end of the Rabbit beta-globin PolyA signal creating SEQ ID NO: 194. SEQ ID NO: 194 and the vector containing SEQ ID NOS: 190 and 191 were digested with KpnI and SalI. The 2024 bp fragment with SEQ ID NO: 194 and the 6603 bp fragment comprising SEQ ID NO: 190 and SEQ ID NO: 191 were isolated by agarose gel electrophoresis and eluted from the agarose using NEB Monarch DNA Gel isolation kit. Ligations of the sticky end fragments were performed with T4 DNA ligase. Successful ligation products were isolated from E. coli transformants and confirmed by restriction digest and Sanger sequencing to confirm the insertion of the promoter and native MTM1 CDS into the vector with appropriate ITRs for creating a self-complementary AAV vector. The vector created this way contains a full ITR, the miniTK promoter, the native MTM1 CDS, the Rabbit beta-globin Poly A signal and an ITR with an appropriate deletion to create a self-complementary AAV vector. The portion of this vector within (and including) the ITRs is provided as SEQ ID NO:208.

To create a similar vector with a miniaturized version of the Desmin promoter, the mini Desmin promoter was synthesized with KpnI site at the 5′ end and NotI site at the 3″ end (SEQ ID NO: 195). Additional bases were added to the synthesized product to enhance the efficiency of restriction digestion (SEQ ID NO: 196). The fragment containing SEQ ID NO: 196 was digested with KpnI and NotI and inserted into a vector containing SEQ ID NO: 184 via the same restriction sites following agarose gel electrophoresis, gel extraction. T4 ligation. This vector (the portion of which within (and including) the ITRs is provided as SEQ ID NO: 197), after sequencing, was determined to have a undesired deletion in the 5″ ITR. However, the insert of SEQ ID NO: 197 was identical to the desired sequence. The miniDes-native MTM1 sequence was PCR amplified using the 5′ primer SEQ ID NO: 213 (tttttGtcGACCCTCTATAAATACCCGCTCTGG) which contains a polyT sequence to aid in restriction digestion, the KpnI site, and the 5′ end of the miniDesmin promoter and the 3″ primer SEQ ID NO. 214 (tttttt cctagg gagTGAGAGACACAAAAAATTCCAACACAC) which contains a polyT sequence to aid in restriction digestion, an Avril site, and the reverse complement of the 3′ end of the Rabbit beta-globin PolyA signal creating SEQ ID NO: 198. SEQ ID NO: 198 and the vector comprising SEQ ID NO: 190 and SEQ ID NO: 191 were digested with KpnI and SalI. The 2185 bp fragment containing SEQ ID NO: 198 and the 6603 bp fragment containing comprising SEQ ID NO: 190 and SEQ ID NO: 191 were isolated by agarose gel electrophoresis and eluted from the agarose using NEB Monarch DNA Gel isolation kit. Ligations of the sticky end fragments were performed with T4 DNA ligase. Successful ligation products were isolated from E. coli transformants and confirmed by restriction digest and Sanger sequencing to confirm the insertion of the promoter and native MTM1 CDS into the vector with appropriate ITRs for creating a self-complementary AAV vector. The vector created this way contains a full ITR, the miniDesmin promoter, the native MTM1 CDS, the Rabbit beta-globin Poly A signal and an ITR with an appropriate deletion to create a self-complementary AAV vector. The portion of this vector within (and including) the ITRs is provided as SEQ ID NO: 199.

### 8.8.1.3 Expression Studies by Cell Transfection

The RD cell line (ATCC CCL-136) was used for our in vitro expression studies. RD cells are derived from patients with Rhabdomyosarcoma, a rare form of pediatric cancer that develops from skeletal muscles. RD cells were maintained in 10% FBS DMEM inside a humidified 37 degrees C. incubator with 5% CO₂ air with serial passage every three to four days following TrypLE non-enzymatic lifting and replating at ¼th density.

24 h prior to transfection, RD cells were lifted with TrypLE, pelleted (7, room temperature, 1400×g), and resuspended in media. Viability was determined by Trypan Blue exclusion using two chambers of a Countess automated cell counter (Thermo Fisher). Average cell density was adjusted to 3.2E5 live cells per mL and 1.6E5 viable cells were plated in 500 uL media. In a 24 well plate.

On the day of transfection, all reagents were warmed to room temperature before use. Plasmid DNA was diluted to 250 ng/uL in TE buffer. Enough reagent was used to transfect 4 wells per plasmid. 100 uL OptiMEM (gibco) plus 6 uL Lipofectamine 3000 (Thermofisher, lot 2170726) was prepared per plasmid. Separately, 100 uL of OptiMEM plus 6 ug of DNA (24 uL of 250 ng/uL diluted plasmid) plus 12 uL 3000 Reagent (Thermo Fisher) were combined. The diluted DNA was then mixed with the diluted Lipofectamine 3000, spun down briefly, and incubated at room temperature for 16 minutes. 57 uL, of the mixture was added to each of 4 wells per plasmid. Some wells of cells were left untransfected to serve as a negative control.

24 hours post-transfection, RD cells were imaged on a BioTek Lionheart for eGFP expression to confirm successful transfection and to estimate % transfection efficiency. A 1 second exposure using the LED intensity setting of 10 was used. After imaging, the cells were washed with 500 uL DPBS. The DPBS was removed and 125 uL TrypLE was added and incubated for 5 minutes at 37 degrees C. in a humidified incubator with 5% CO2. The cells were triturated to resuspend and pelleted at 140×g at 4 degrees C. for 7 minutes. 5 mL of 2× lysis was buffer was prepared in sterile filtered dH2O with 10×RIPA buffer (Cell Signaling) with 1 tablet of Roche EDTA-free Mini Complete Protease Inhibitor. 12 uL of the 2× lysis buffer was added to the cell pellet. The lysate was vortexed briefly and spun down. Samples, lysed or unlysed, were stored at minus 80 degrees C.

Plasmids used: In addition to the MTM1 expression constructs (Seq 1 to 9) certain reference plasmids were used. Plasmid 7701591057, a fully-synthesized plasmid vector, which contains AAV2 ITRs and eGFP under the control of the CAG promoter and the Rabbit beta-globin PolyA signal was used as a transfection control for fluorescently visualizing eGFP and percent of cells successfully transfected as well as a negative control for antibody-mediated MTM1 detection. pCDNA3.1+C/(K) DYK with human native MTM1 under the control of the CMV promoter, an in-frame DYK epitope tag, and a bovine Growth Hormone PolyA signal. This was obtained from Genscript.

### 8.8.1.4 Automated Western Analysis

In vitro expression of MIMI was analyzed using the ProteinSimple Jess automated western blot. Jess was utilized to fully automate capillary loading, protein separation, incubation, and detection. Protein lysates from transfected and untransfected RD cells were diluted 1:4 in 0.1× Sample Buffer+1× Fluorescent Master Mix (ProteinSimple, PS-ST01EZ-8) and 3 uL was loaded onto a separation module (ProteinSimple, SM-W004). Capillaries were incubated with an MTM1 polyclonal antibody at a 1:15 dilution (Proteintech, 13924-1-AP) and a Secondary Mouse Antibody conjugated with HRP (ProteinSimple. DM-001). In addition to an immunoassay, the Total Protein Detection module (ProteinSimple, DM-TP01-1) was included to allow for normalization of MTM1 expression by total protein load. Total protein and MTM1 were detected by the chemiluminescence channel.

### 8.8.2. Results

Results are shown in FIG. 20. Total human myotubularin protein levels were quantified in RD muscle cells following transfection with 9 different MIMI containing expression plasmids. All Mtm1 expression plasmids expressed levels of MTM1 protein significantly greater than controls (untransfected and GFP transfected controls). The CAG promoter expressed higher MTM1 protein levels in RD cells compared to Desmin promoter containing plasmids. Codon optimization of the MTM1 transgene using Gene Art. Genscript. and Eurofins algorithms had minimal impact on expression of MTM1 protein in RD cells. These findings indicate that the CAG promoter drove higher levels of MTM1 protein expression in human RD muscle cells in vitro compared to plasmids containing the Desmin promoter.

### 8.9. Example 9: Anc80 Variants

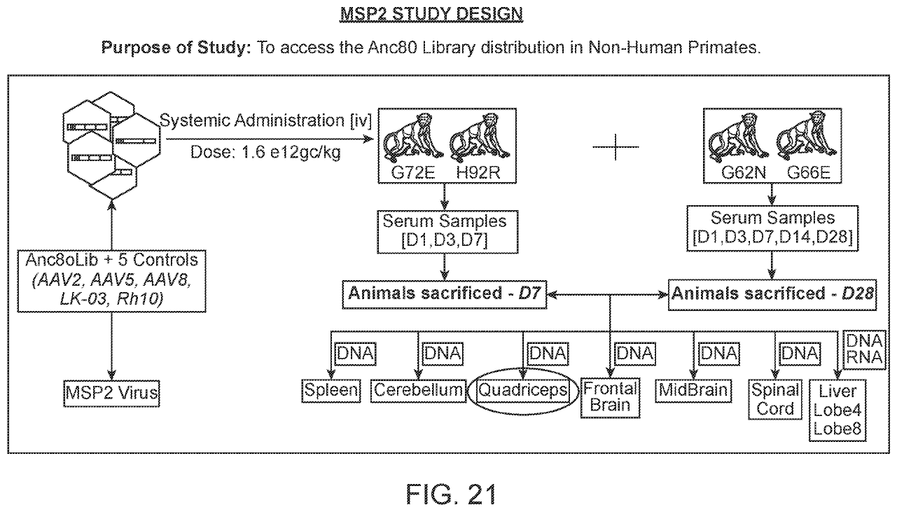

The distribution of AA Vs containing the Anc80 library capsids (SEQ ID NO: 132) systematically administered to non-human primates (NHPs) as outlined in FIG. 21 and Table X. From this study, Applicant aimed to identify capsids in the Anc80 library having preferred distribution patterns in non-human primates.

In short, AAV capsids of the Anc80 library and 5 control capsids (AAV2, AAV5, AAV8, LK-03, Rh10) were systemically administered to four NHPs by i.v. injection at the dose of 1.6×10¹² gc/kg. In the first two animals (G72E. H92R), serum samples were obtained on days 1, 3, and 7, and other tissue samples were obtained on day 7 after the animals were sacrificed. In the second two animals (G62N, G66E), serum samples were obtained on days 1, 3, 7, 14, 28 and other tissue samples were obtained on day 28 after the animals were sacrificed.

TABLE 9
Study design for Anc80 capsid library.

| | | Route of | | | | Post |
|---|---|---|---|---|---|---|
| Group | No. of | Admini- | | | Dosage | Procedure |
| No. | Animals | stration | Animals | AAVS | (vg/kg) | Analysis |
| 3 | 2 | IV | G72E and | Anc80 | 1.6E12 | 7 days |
| | | | H92R | Library | | |
| 2 | 2 | IV | G62N and | Anc80 | 1.6E12 | 28 days |
| | | | G66E | Library | | |

Individual tissues, including the liver and major skeletal muscles of the hind limb, heart, and diaphragm, were collected at the time of necropsy. For RNA, tissues were immediately placed into the preservative RNAlater, after which the RNAlater was removed and the tissue flash frozen For DNA, tissues were homogenized in a Qiagen Tissuelyser II (20 rps for 2 min) in lysis buffer from the Qiagen Dneasy Blood and Tissue Kit or the Qiagen RNeasy Lipid Tissue Mini Kit following the standard Qiagen protocol. Samples were eluted in 50 μL of buffer. For tissue expression analysis. NGS was performed on RNA harvested from non-human primate tissue following IV infusion of the Anc80 library and reverse transcribed into cDNA for analysis. As noted above, individual tissues (i.e., the liver and major skeletal muscles of the hind limb (quad)), were collected at the time of necropsy. Tissues were immediately placed into the preservative RNAlater, after which the RNAlater was removed and the tissue flash frozen.

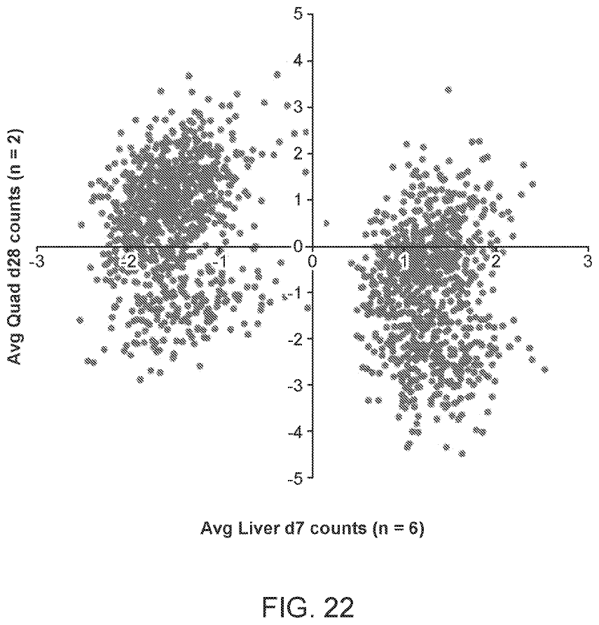

The viral DNA and RNA of each of the Anc80 variants were measured and enrichment of Ane80 variants in the quadricep muscle and the liver was analyzed. PCR amplified products corresponding to the bar code of the vector were analyzed via Illumina sequencers, either MiSeq or NovaSeq, to quantify the presence of each variant in tissue or test article. These counts were normalized to one million reads. Tissue enrichment was calculated by dividing tissue counts per million by test article counts per million. The base two logarithm is often used to present this last calculation. Enrichment data of Anc80 variants in the quadricep muscle (y-axis) and the liver (x-axis) are presented as individual dots in the plot in FIG. 22.

In the previous study described in WO2019217911, which is incorporated by reference in its entirety herein, it was demonstrated that amino acid at the position “P3” corresponding to amino acid 266 of Anc80 capsid protein can affect liver tropism of the AAVs containing the capsid protein. Specifically, capsids having a glycine (G) at P3 tend to have “liver on” whereas capsids having alanine (A) at P3 tend to have “liver off” properties.

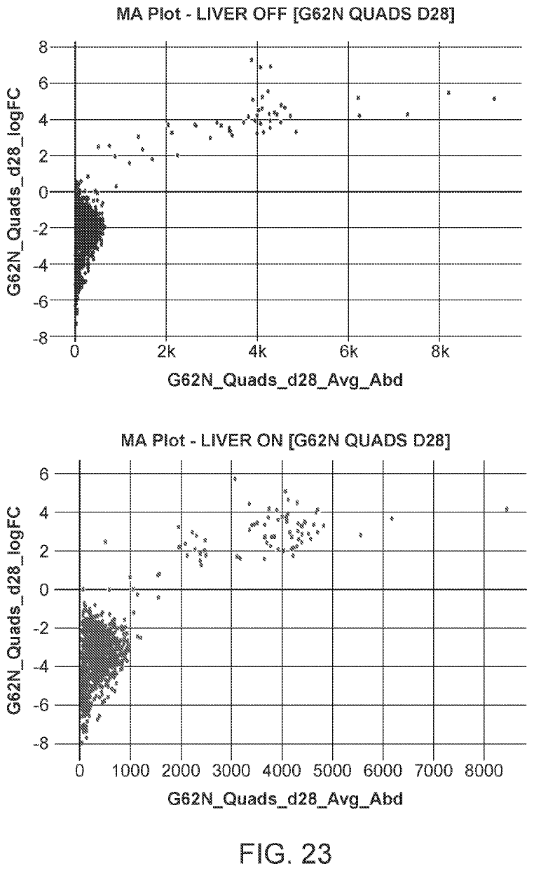

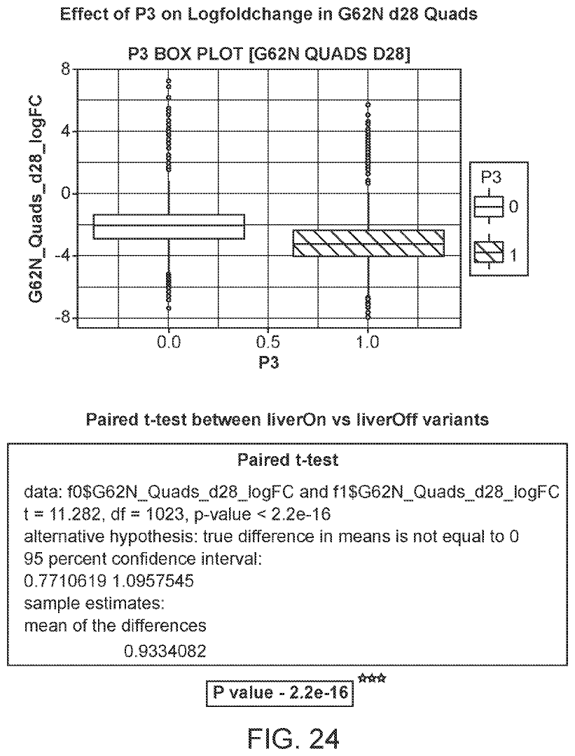

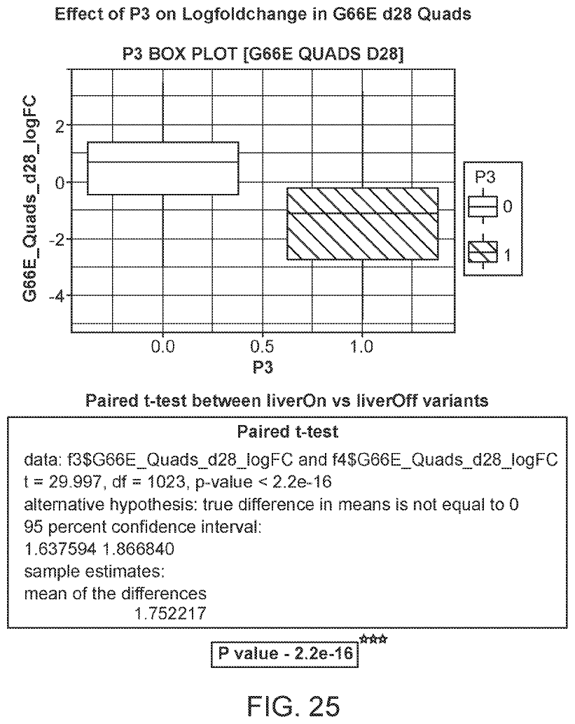

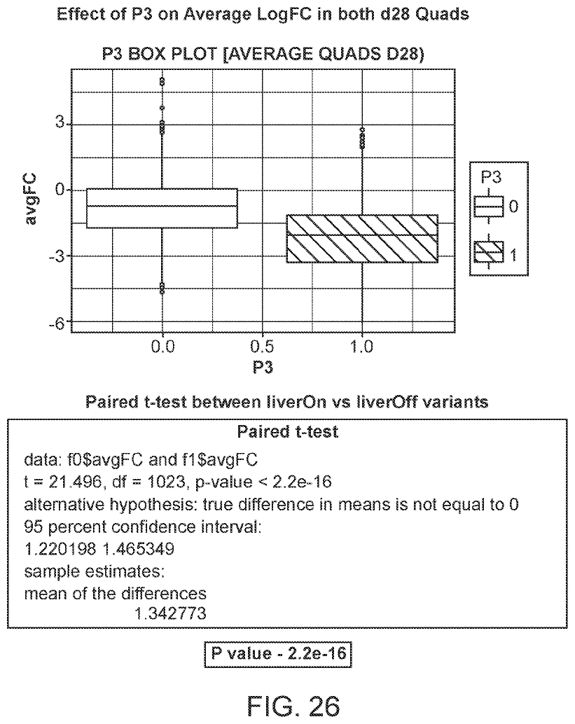

Anc80 capsid variants having G (“liver on”) or A (“liver off”) at P3 position respectively were separately analyzed and their log fold changes (log (FC)) in the quadricep muscle are plotted in FIG. 23. FIG. 23 shows that most of the Anc80 variants, both liver on and liver off, have negative log fold changes in tissues compared to target input (the pool of AAV variants that were administered). The data were further analyzed in FIG. 26 by box plotting log fold changes of liver-off Anc80 capsids (left) compared to liver-on Anc80 capsids (right). Paired t-test between liver-on and liver-off variants show that there is significant difference between the two groups. FIG. 26 provides the data of two experimental animals on average, and FIG. 24 (G62N) and FIG. 25 (G66E) provide data from individual animals.

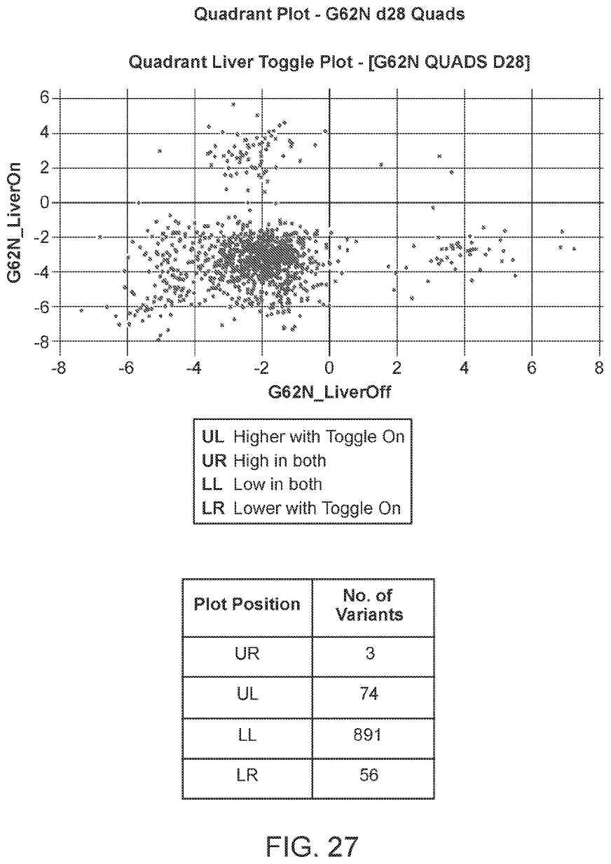

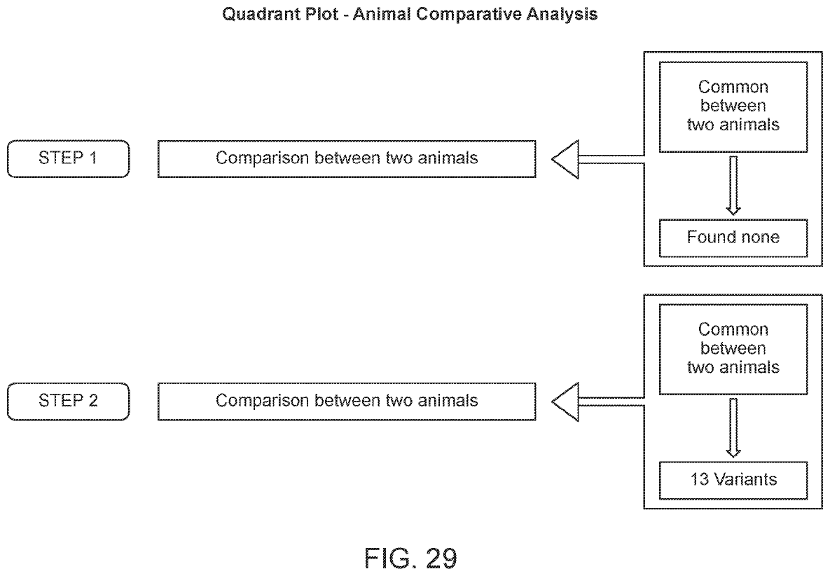

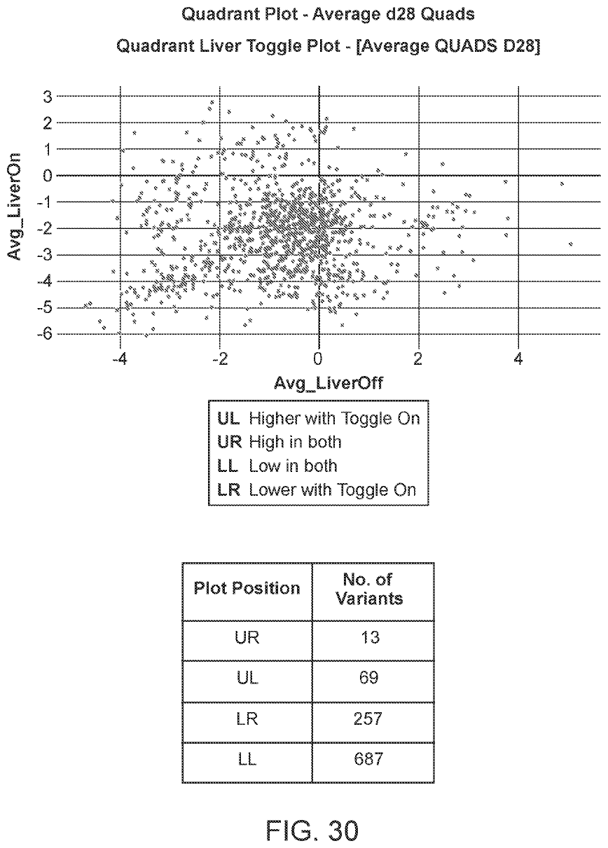

The quadricep enrichment data for the two animals are also provided in the quadrant plots of FIG. 27 (G62N) and FIG. 28 (G66E). The average data of the two animals are provided in FIG. 30. In the quadrant plots, each dot represents the quadricep enrichment of an Anc80 variant with G (“liver on”) (y-axis) or A (“liver off”) (x-axis) at P₃ position. Dots on the upper left box (UL) represent Anc80 variants with higher quadricep enrichment with G (“liver on”), dots in the upper right box (UR) represent Anc80 variants with high quadricep enrichment in both G (“liver on”) and A (“liver off”), dots in the lower left box (LL) represent Anc80 variants with low quadricep enrichment in both G (“liver on”) and A (“liver off”), and dots in the lower right box (LR) represent Anc80 variants with lower quadricep enrichment with G (“liver on”). The enrichment data from two different animals in each group were individually compared or averaged as illustrated in FIG. 29.

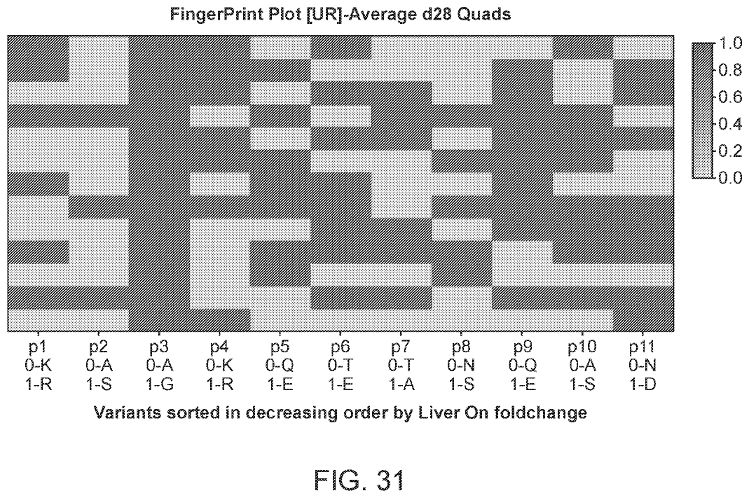

Anc80 variants having different amino acids at other positions—P1 (amino acid 168 of Anc80 capsid). P2 (amino acid 205), P3 (amino acid 266), P4, . . . . P10 (amino acid 587), P11 (amino acid 609)—were also analyzed and the results are provided in FIG. 31. Alternative amino acids at each variation site are summarized in the below table.

TABLE 10

| | P1 | P2 | P3 | P4 | P5 | P6 | P7 | P8 | P9 | P10 | P11 |
|---|---|---|---|---|---|---|---|---|---|---|---|
| Amino acid | 168 | 205 | 266 | 311 | 411 | 460 | 493 | 562 | 576 | 587 | 609 |
| position in | | | | | | | | | | | |
| Anc80 | | | | | | | | | | | |
| Alternative | K/R | A/S | A/G | K/R | Q/E | T/E | T/A | N/S | Q/E | Q/S | N/D |
| amino acids | | | | | | | | | | | |

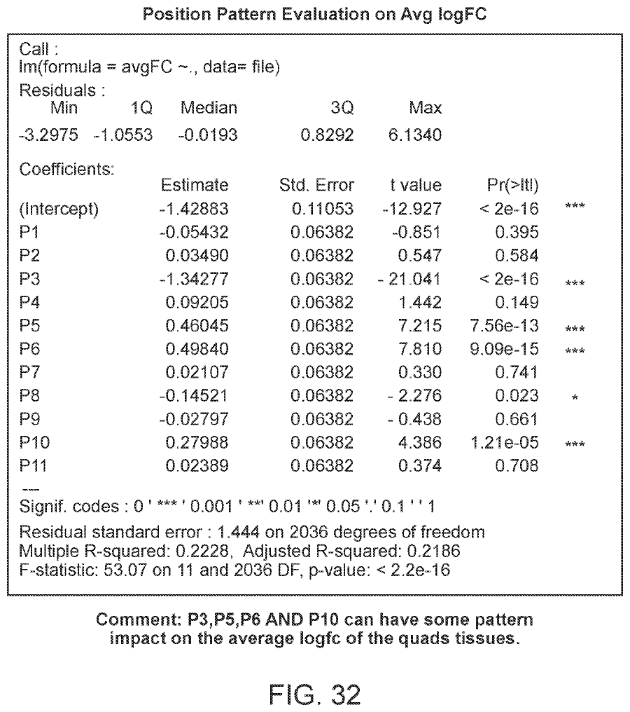

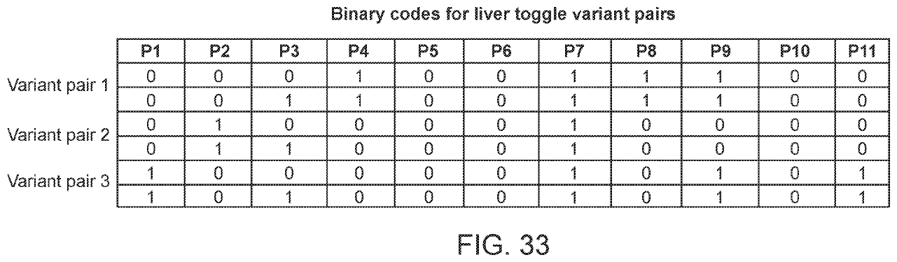

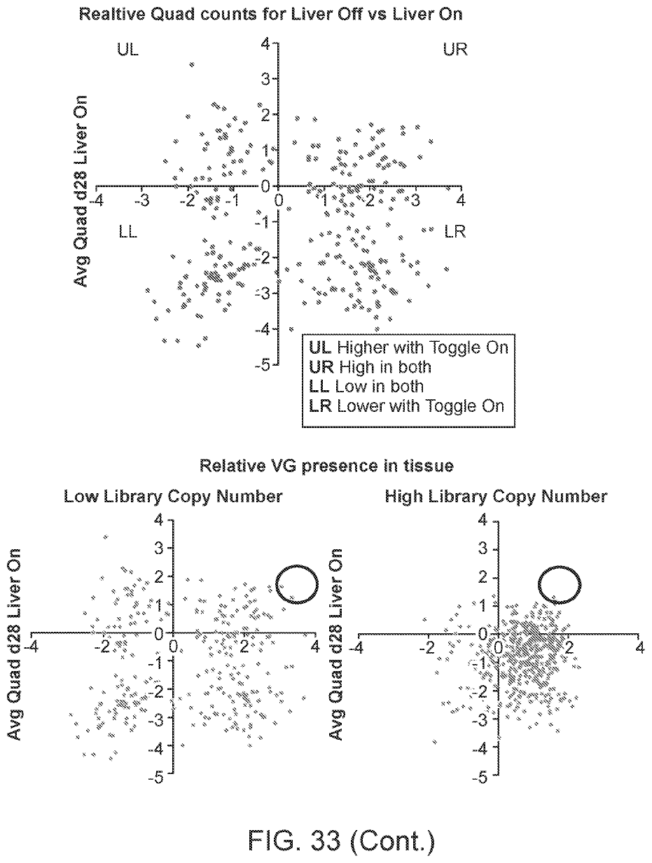

In FIG. 31, Anc80 variants were sorted by decreasing order by liver-on fold change, and alternative amino acids at each variation site are color coded. The relationship of the variations at the sites and the liver-on fold changes were then analyzed as provided in FIG. 32. The analysis showed that variations at P3, P5, P6, and P10 have some pattern impact on the average log fold changes (log (FC)) of tropism to the quadricep muscle. FIG. 33 shows exemplary amino acids at the P1 to P11 positions in three liver-toggle variant pairs. Two capsids in each pair differs only at one of the P1 to P11 positions, and the one amino acid difference makes one capsid liver-on and the other capsid liver-off.

With these analysis, 6 Anc80 variants (Anc80-55, Ane80-129, Anc80-156, Anc80-751, Anc80-1029, and Anc80-1712) were selected from the Anc80 library for the high muscle tropism in the quadricep.

### 8.10. Example 10: Deco1 Targeting Peptide Enhances Muscle Delivery in Anc80 Variants

To study the effect of a targeting peptide on Anc80 variants, a targeting peptide having SEQ ID NO: 1 (“deco1”) was inserted into VR VIII of Anc80 variants (Anc80L65 (SEQ ID NO: 44895), Anc80L65^{MuT1} (SEQ ID NO: 44896), Anc80-55 (SEQ ID NO: 44885), Anc80-129 (SEQ ID NO: 44887), Anc80-156 (SEQ ID NO: 44889), Anc80-751 (SEQ ID NO: 44916), Anc80-1029 (SEQ ID NO: 44917), and Anc80-1712 (SEQ ID NO: 44893). The Anc80 variants with the deco 1 insertion are referred to here as Anc80L65^{deco1} (SEQ ID NO: 44897), Anc80L65^{mut1deco1} (SEQ ID NO: 44898), Anc80-55^{deco1} (SEQ ID NO: 44886), Anc80-129^{deco1} (SEQ ID NO: 44888), Anc80-156^{deco1} (SEQ ID NO: 44890), Anc80-1712^{deco1} (SEQ ID NO: 44894), and Anc80-751^{deco1} (SEQ ID NO: 44891), Anc80-1029^{deco1} (SEQ ID NO: 44892).

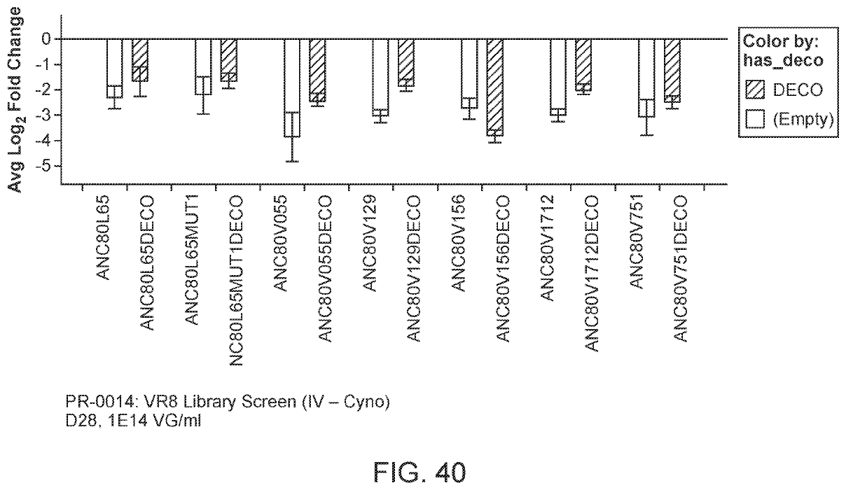

The Anc80 variants with or without deco1 peptide were then administered via IV injection to non-human primates at a concentration of 1×10¹⁴ vg/ml. The variants targeted to the muscles were quantified and the log 2 fold changes of tissue enrichment are show in FIG. 40. In some cases, the tissue enrichment is the ratio of the amount of each individual variant in the tissue divided by the amount of that same variant in the pool of AAV variants administered.

The results show that insertion of deco1 peptide increases muscle tropism of Anc variants. Six (6) out of 7 Anc80 variants with a deco1 insertion at VR VIII enhanced muscle delivery of Anc80 variants (FIG. 40). Tissue enrichment is the ratio of the amount of each individual variant in the tissue divided by the amount of that same variant in the pool of AAV variants administered.

### 8.11. Example 11: Muscle Targeting of AAV Capsids Including RGD Peptides (“VRVIII Library”)

A library of AAVs (VRVIII library) containing capsids modified to comprise a unique targeting peptide inserted into the VR VIII region of the capsid protein was generated. The modified capsid proteins include one of 44,617 unique 7-mer peptides between Q588 and A589 of AAV9^{mut1} (SEQ ID NO: 163) (“VRVIII library”). The sequences of the 7-mer peptides are provided as SEQ ID NOs: 238-44,853 (the “RGD peptide library” of as described in Table 27).

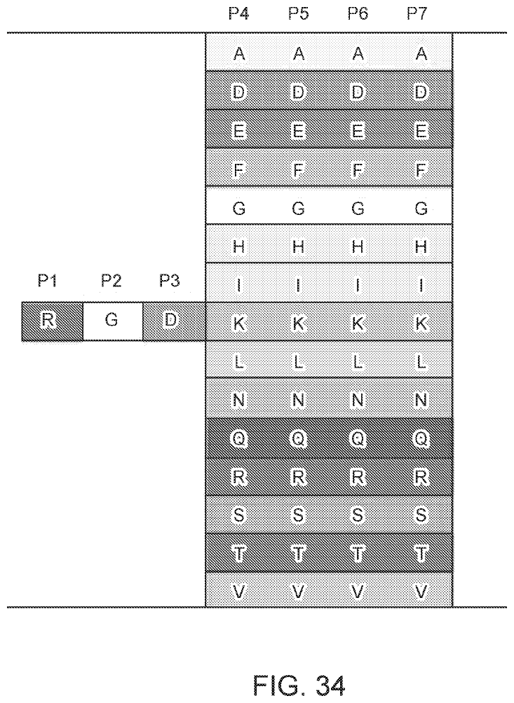

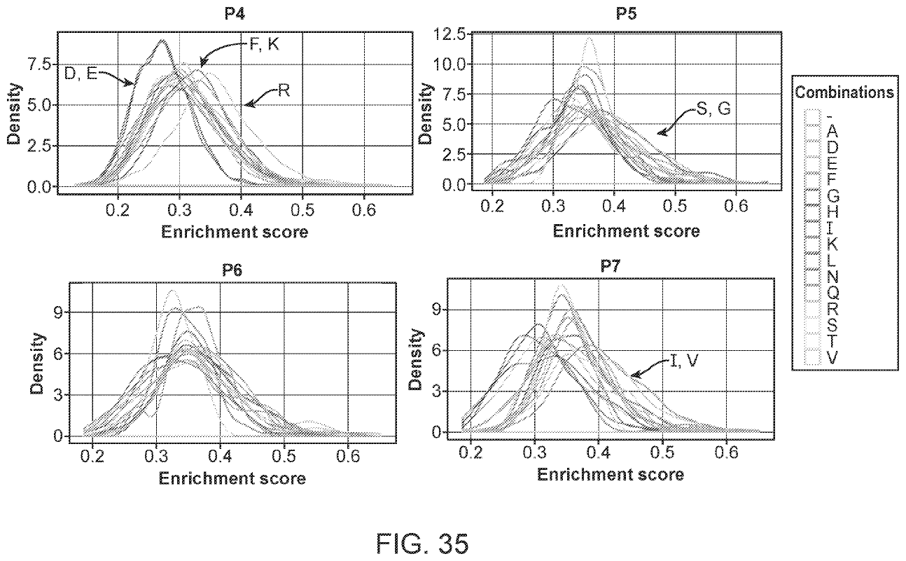

The seven amino acid positions of the targeting peptides inserted between (2588 and A589 of AAV^{mut1} VP1 capsid sequence are identified as “Y1, Y2, Y3, Y4, Y5, Y6, and Y7” in FIG. 34. The 7 amino acid positions (P1, P2, P3, Y4, P5, P6, P7) of FIG. 34 correspond to RGDX₇X₈X₉X₁₀ within X₁X₂X₃RGDX₇X₈X₉X₁₀ described as a targeting peptide in the present disclosure, where “Y1” is “R”, “Y2” is “G”, “Y3” is “D”, “Y4” is “X₇”, “Y5” is “X₈”, “Y6” is “X₉”, and “Y7” is “X₁₀” of X₁X₂X₃RGDX₇X₈X₉X₁₀ (SEQ ID NO: 44915). The peptides have amino acid residue “R” at the Y1 position, amino acid residue “G” at the Y2 position, and amino acid residue “D” at the Y3 position. Further, the peptides have amino acid residues “SAQ” as X₁X₂X₃ which are original amino acids positioned upstream of the insertion site (amino acid positions 586, 587 and 588) in AAV9mut1 VP1 capsid. Thus, capsid proteins in the VRVIII library include a targeting peptide having a sequence of “SAQRGD X₇X₈X₉X₁₀” at VRVIII.

The targeting peptides in the library include one of 15 amino acids (A, D, E, F, G, H, I, K, L, N, Q, R, S, T, V) at Y4, Y5, Y6, and Y7 positions as illustrated in FIG. 34. Random combinations of the 15 amino acids at Y4, Y5, Y6, and Y7 positions allow creation of 50,625 (15\hat{ }4) unique targeting peptide sequences. The 50,625 unique targeting peptide sequences were then quality filtered to remove variation, resulting in 40,617 unique targeting peptide sequences.

AAVs containing the modified AAV capsid proteins were produced using HEK293 cells containing an adenoviral helper gene plasmid construct and a plasmid containing the AAV rep expression cassette and a capsid coding sequence. The modified capsid coding sequence was flanked by the AAV ITR sequences to generate AAVs containing the modified capsid coding sequence encapsidated in the modified capsid proteins. After production of the AAVs, the AAV samples were analyzed by next-generation sequencing using Illumina reagents and hardware. Viral genomic DNA containing the capsid coding sequence was isolated from viral supernatant and sequenced. The analysis confirmed the presence of 44,617 capsids.

To identify modified capsid proteins that provide the desired AAV tropism (e.g., muscle targeting) in NHPs, AAVs containing the modified AAV capsids were administered to cynomolgus monkeys by IV administration. AAV containing the wild-type AAV9 capsids was included in the mixture for comparison. A control animal treated with a buffer was also included.

TABLE 11

| Group | | | Dose | | Volume |
|---|---|---|---|---|---|
| # | N | Treatment | (vg/kg) | Route | (mL) |
| 1 | 1 | TB-0017 (buffer) | NA | IV | 10 |
| 2 | 3 | TV-0080 (AAVs with | 5e13 | IV | 10 |
| | | capsids modified with | | | |
| | | 50,625 RGDX₁X₂X₃X₄ | | | |
| | | targeting peptides) and | | | |
| | | ATV-0048 (AAV with | | | |
| | | wild type AAV9 capsid | | | |
| | | protein) | | | |

Prior to the AAV administration blood samples (2.0 mL) were collected from the monkey's femoral, saphenous, or other available vein via direct venipuncture for AAV neutralizing antibodies and IgG evaluation. Blood was placed into a serum separator tube and serum was collected into 1 aliquot. The aliquot was labeled with “AA V NAb & IgG”. Serum sample was stored in −80° C. until shipment.

TABLE 12
Blood Collection

| | AAV NAbs & IGg |
|---|---|
| | (Affinia) |
| Study Day | 2 mL Blood for Serum |
| Pre-dose Day −28 | X |
| Day 1 (Prior to injection) | X |

On Day 29 after AAV administration, all study animals, were euthanized and underwent a tissue collection in accordance with PP LLC euthanasia procedures. Their tissue samples were collected with care to avoid contamination among samples. Viral genomic DNA was isolated from the tissue samples and sequenced to identify the targeting peptides in the AAVs localized in each tissue. Using the sequence data, enrichment scores for each tissue were calculated for each AAV variant containing a unique targeting peptide. In particular, the AAV variants including each targeting peptide were ranked based on a mean log fold-change tissue score (Column C of Table 27). Table 27 also includes tissue enrichment scores for each individual tissue analyzed. After determining a sequence count from sequence read data (FASTQ) following sequencing, AAV variants containing a unique targeting peptide were counted and normalized, followed by performing a tissue enrichment analysis for each tissue. Tissue enrichment analysis included Sequence Activity Relationship (SAR) analysis, Network analysis, and structural modeling. SAR analysis identified particular amino acid at a specific capsid position or sequence motifs (combination of amino acids at several positions) that significantly affects tissue tropism. Network analysis identified modules of variants that shared amino acid or peptide sequences of top performing variants. Structural modeling provided understanding of the structural effect of significant amino acids at specific capsid positions for mechanistic hypothesis formulation.

A mean log fold-change tissue enrichment score is based on the combination of amino acid residues present in the modified sequence within VR VIII. The formula used to determine a tissue enrichment score is provided below:

$$Tissue Enrichment score = log 2 FC = log 2 {MN}_{Tissue} - log 2 {MN}_{TestArticle}$$

$$scaled log 2 FC = ( log 2 FC - min ( log 2 FC ) ) / ( max ( log 2 FC ) - min ( log 2 FC ) )$$

After performing a SAR analysis, network analysis, and structural modeling, the RGD peptide library variants were ranked and a tissue enrichment score was calculated. A higher individual tissue enrichment score of columns F, G, H, I, J, K, and L equate to a higher amount of the AAV variant found in that particular tissue. Individual tissue enrichment scores for each AAV variant are shown in columns F, G, H, I, J, K, and L of Table 27.

By analyzing muscle enrichment scores corresponding to the targeting peptides having variants having an “R” amino acid residue at Y4 position (or “X₇” in X₁X₂X₃RGDX₇X₈X₉X₁₀) were found to have higher muscle enrichment scores compared to other variants having different amino acid residues at the position. A higher enrichment score is equated to a higher amount of the modified AAV capsid protein found in that particular tissue. Based on the finding, Y4 was identified to be an important position that affects muscle tropism, with amino acid residue “R” in position 4 being particularly effective. As shown in FIG. 35, the density plot shows highest tissue enrichment when Y4 is “R” as compared to other amino acid residues F, K, D, E, S, G, I, or V. Additionally, after fixing R at position 4, amino acid residues S or G at position 5 (or “X₈” in X₁X₂X₃RGDX₇X₈X₉X₁₀) (P5) and I or V at position 7 (or “X₁₀” in X₁X₂X₃RGDX₇X₈X₉X₁₀) were found to lead to higher muscle enrichment scores.

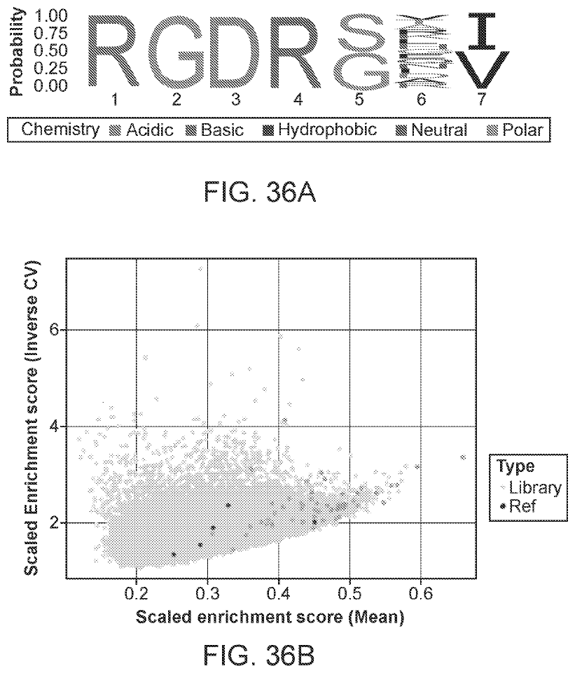

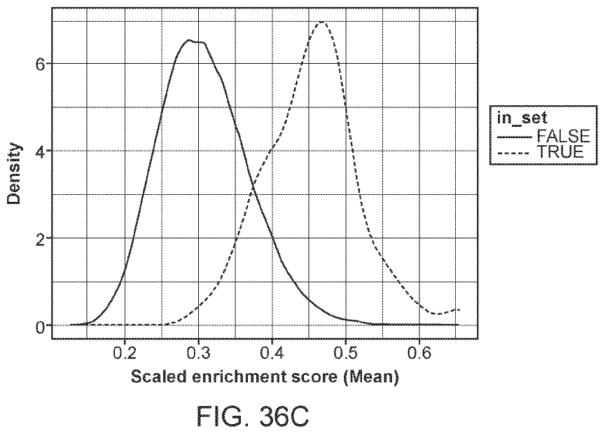

Based on the analysis the muscle-tropic sequence motif was identified as provided in FIG. 36A. FIG. 36B is a scatter plot showing muscle enrichment scores of variants (an inverse coefficient of variation, which is the mean divided by the standard deviation). Each dot represents a single variant with a unique targeting peptide, and dots corresponding to variants with the muscle-tropic motif of FIG. 36A are colored in red. The muscle enrichment scores of the variants with and without the muscle-tropic motif were further analyzed as provided in FIG. 36C, which shows that there was significant separation between targeting peptide with the muscle-tropic motif and targeting peptide without the motif.

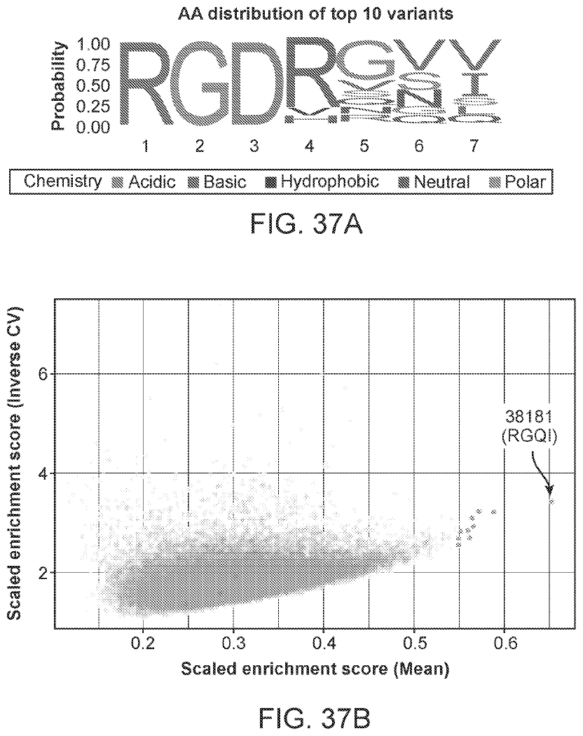

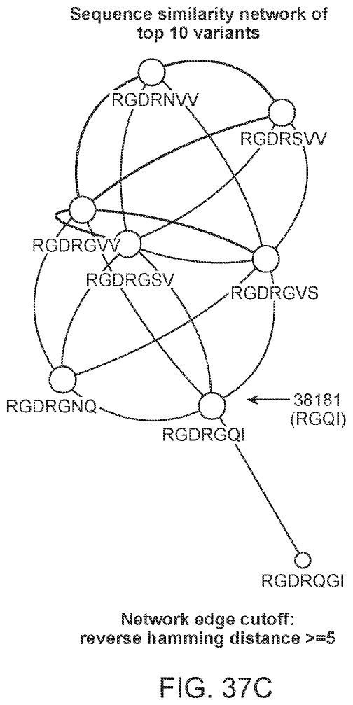

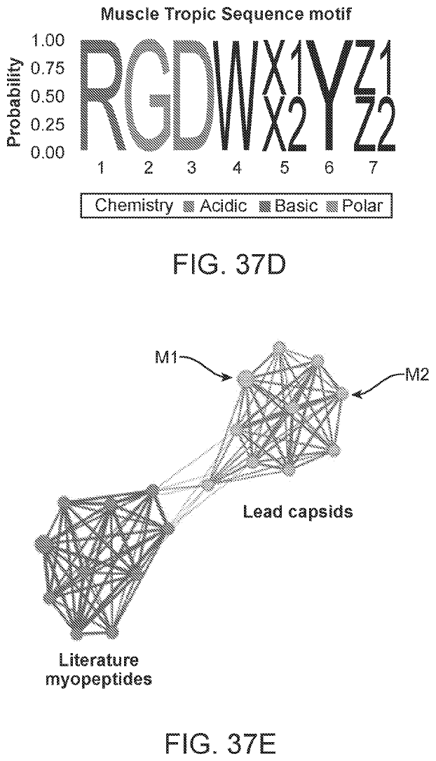

FIG. 37A shows amino acid probability distribution of the targeting peptides that provided top 10 muscle enrichment (see Table 27). Many of the top 10 peptides included amino acid residue “R” at position 4, amino acid residues S or G at position 5 and I or V at position 7 showing significant sequence similarity to the muscle-tropic motif in FIG. 36A. In particular, the top targeting peptides (FIG. 37B) belong to a muscle-tropic sequence motif by having R, G, Q, and I, at Y4, Y5, Y6, and Y7 position, respectively. Further, the top 10 peptides bad highly similar sequences to each other. 8 out of 10 top variants were connected in a sequence similarity network. The network plot (FIG. 37C) shows a capsid represented by a “dot”, where the lines connecting various dots represent sequence connectivity between the capsids. The sequence similarity network shows that SEQ ID NO: 238 (“ATLVT013XX38181”) was the top targeting peptide for muscle tropism and has a sequence similar to other targeting peptides providing high muscle enrichment. In the network plot, each dot/line is a AAV variant. The radial axis represents the tissue enrichment score for a particular tissue. The most inner circle represents a “low tissue enrichment”, while an outer circle represents a “high tissue enrichment”. Network plots are useful for comparing the performance of a small number of variants in multiple tissues. Similar analysis was performed in muscle tropic sequence motifs (FIG. 37D) including a network comparison to literature myopeptides (FIG. 37E), thereby showing the differences between the targeting peptides descried herein and those described in the art.

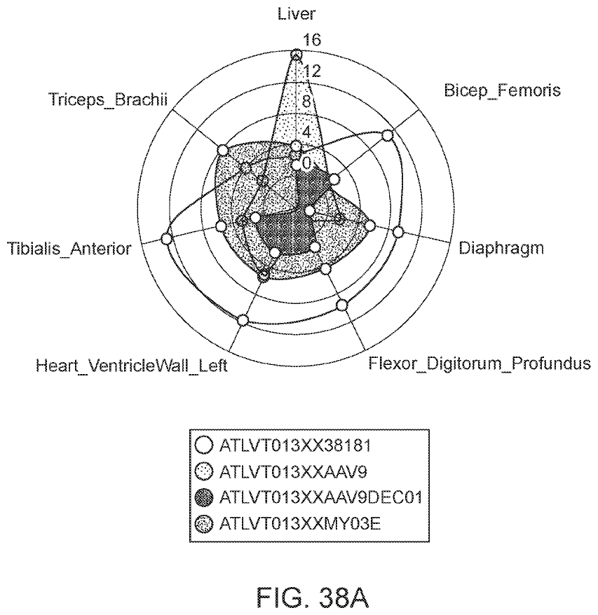

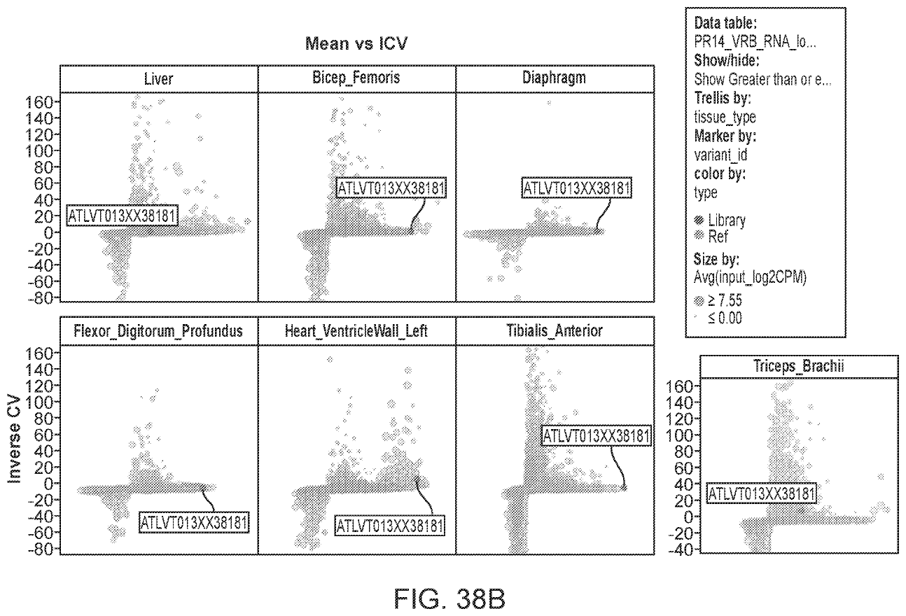

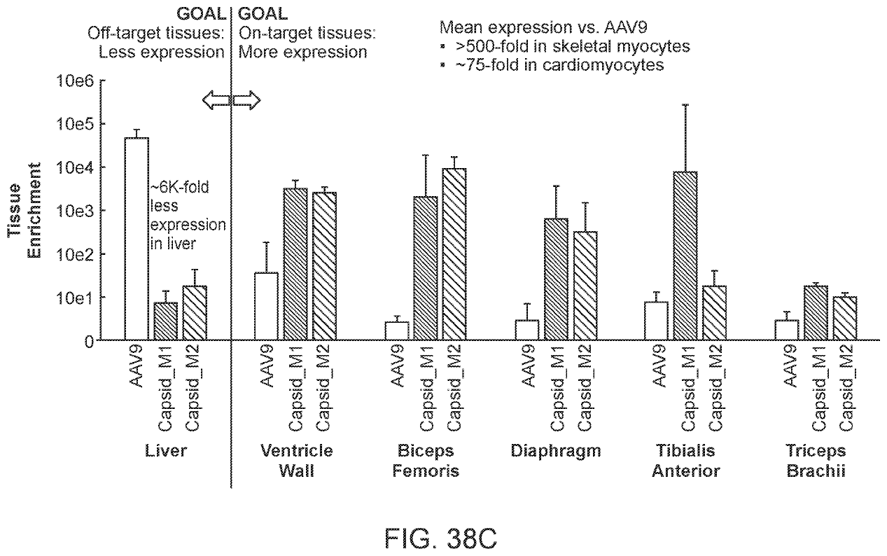

FIG. 38A shows enrichment of four AAV variants (AAV9^{mut1} containing SEQ ID NO: 238 (“ATLVT013XX38181”); wild-type AAV9, AAV9^{deco1} (AAV9 capsid with the deco1 peptide), and AAV9 with SEQ ID NO. 44880). FIG. 38B is a scatter plot showing tissue enrichment scores for each tissue region assessed. The network analysis and scatter plot show that AAV9^{mut1} containing SEQ ID NO: 238 (“ATLVT013XX38181”) outperformed wild-type AAV9, AAV9^{deco1}, and AAV9 with SEQ ID NO: 44880 for muscle tropism. Further, AAV9^{mut1} containing SEQ ID NO: 238 maintained the liver-detargeting phenotype AAV9^{mut1} as compared to wild-type AAV9, showing lower liver enrichment. The targeting peptide SEQ ID NO: 238 (“ATLVT013XX38181”) consistently ranked at the top for target enrichment in all muscles except for the triceps.

FIG. 38C shows a plot of tissue enrichment scores for liver and the indicated muscle tissues for AAV9 and AAV9 comprising an M1 (RGDRGQI (SEQ ID NO: 238) targeting peptide located in VR VIII between amino acids at positions 588 and 589 or an M2 (RGDRSVV (SEQ ID NO: 239)) targeting peptide located in VR VIII between amino acids at positions 588 and 589. Capsids comprising either an M1 targeting peptide or an M2 targeting peptide showed a 4-log reduction in tissue enrichment in the liver, which indicates a 4-log reduction in liver tropism, as compared to AAV9 (see FIG. 38C). Additionally, capsids comprising either an M3 targeting peptide or an M2 targeting peptide also showed increased tissue enrichment (i.e., increased tropism) in each of the muscle tissues analyzed as compared to AAV9 (see FIG. 38C). The increased muscle tropism in capsids comprising either an M1 targeting peptide or an M2 targeting peptide ranged from 10-100 fold increased expression compared to AAV9.

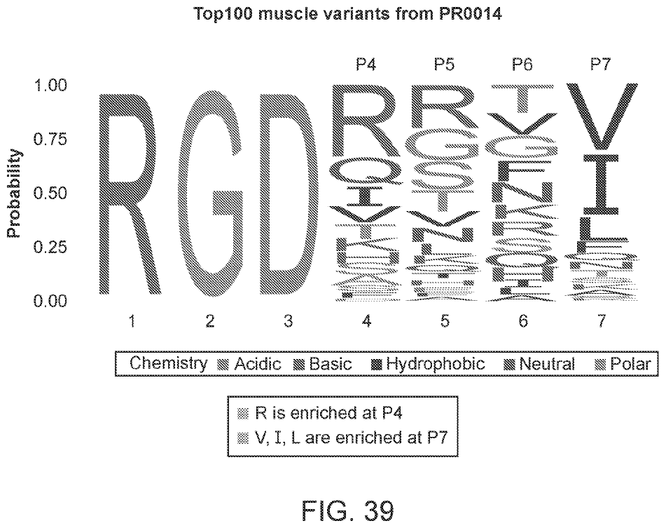

FIG. 39 shows amino acid probability distribution of the targeting peptides that provided top 100 muscle enrichment. Similar to the earlier data with top 10 variants, amino acid “R” is enriched at position 4 (Y4), and amino acids “V, I, and L” are enriched at position 7 (Y7).

From this study, several targeting peptides that can drastically improve muscle delivery in the heart, diaphragm, biceps femoris, flexor digitorum, tibialis anterior, triceps, Quadriceps and gastrocnemius, were identified.

### 8.12. Example 12: Muscle Targeting of AAV Capsids Including RGD Peptides (VRVIII Mini-Library)

A library of AAVs (VRVIII mini-library) containing capsids modified to comprise a different set of targeting peptides inserted into the VR VIII region of the capsid protein was generated.

The modified capsid proteins include one of 22 unique targeting peptides (SEQ ID NO: 44859-44878, 44879, and 44883) between Q585 and A589 of AAV9 (SEQ ID NO: 61). The modified capsid proteins include the targeting peptide having the sequence of X₁X₂X₃RGDX₇X₈X₉X₁₀ in place of “SAQ”, the original amino acids at positions 586, 587 and 588 in AAV9 VP1 capsid protein. In some modified capsid proteins, X₁X₂X₃ has the original sequence, “SAQ” of the AAV9 capsid protein (SEQ ID NOs.: 44881-44882, 44911, 44912, 44913, 44918-44919), whereas in other modified capsid proteins, X₁X₂X₃ has a sequence different from the original “SAQ” (SEQ ID NOs: 44859-44878, 44879, 44880, 44883, and 44910).

AAVs containing the modified capsid proteins of the VRVIII mini-library were generated and tested as described in Example 11. They were administered to the NHPs and muscle enrichment of the variants was analyzed.

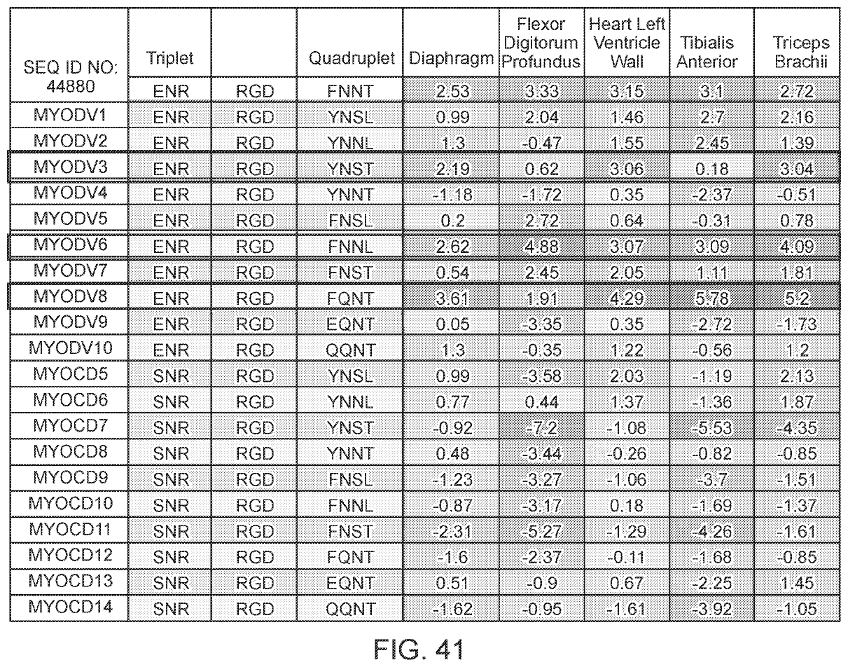

FIG. 41 shows a list of 20 targeting peptides (myoDV1-myoDV10 and myoCD5-myoCD14) in the VRVIII mini-library. FIG. 41 shows tissue enrichment scores for each of the 20 targeting peptides in muscle tissue (diaphragm, flexor digitorum profundus, heart left ventricle wall, tibialis anterior, and triceps brachii). Higher tissue enrichment score values represent greater muscle tropism, with MYODV6 and MYODV8 having the highest averages tissue enrichment score for all muscle tissue regions.

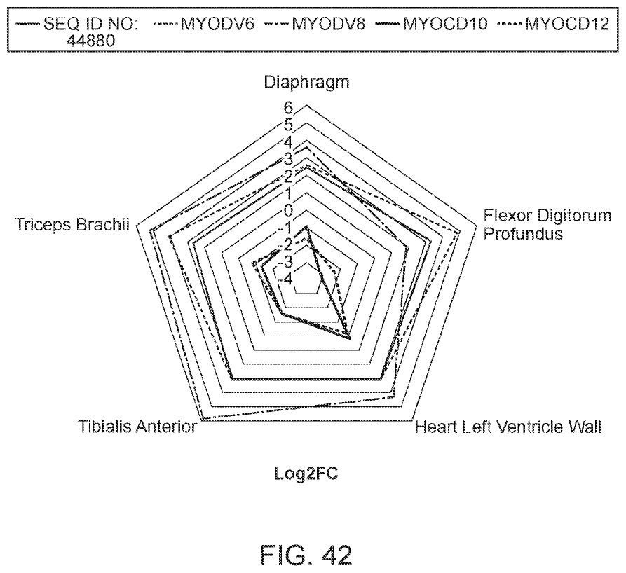

FIG. 42 shows a network plot showing log 2 fold change of tissue enrichment for each of the muscle tissues analyzed for the targeting peptide having an amino acid sequence of SEQ ID NO: 44880. MYODV6, MYODV8, MYOCD10, and MYOCD12 targeting peptides. Surprisingly, as shown in the network plot, MYODV6 and MYODV8 each have the highest overall log 2 fold change of tissue enrichment in each muscle tissue compared to MYOCD10 and MYCD12 (which contain amino acid residues “SNR” (triplet) preceding “RGD” within the targeting peptide. Additionally, MYODV6 and MYODV8 each have a higher overall log 2 fold change of tissue enrichment in each muscle tissue compared to a targeting peptide having an amino acid sequence of SEQ ID NO: 44880.

### 8.13. Example 13: SAR Analysis of Modified AAV Capsid Proteins Identifies Cardiac-Tropic Capsids

Using the data from Example 11, SAR analysis was performed with the aim of identifying modified AAV capsid proteins having different tropism for different types of muscle tissue. For example, SAR was used to identify modified AAV capsid proteins that had increased enrichment in cardiac muscle tissue as compared to skeletal muscle tissues. The reverse analysis was also performed, that is. SAR was used to identify modified AAV capsid proteins that had increased enrichment in skeletal muscle tissue as compared to cardiac muscle tissue.

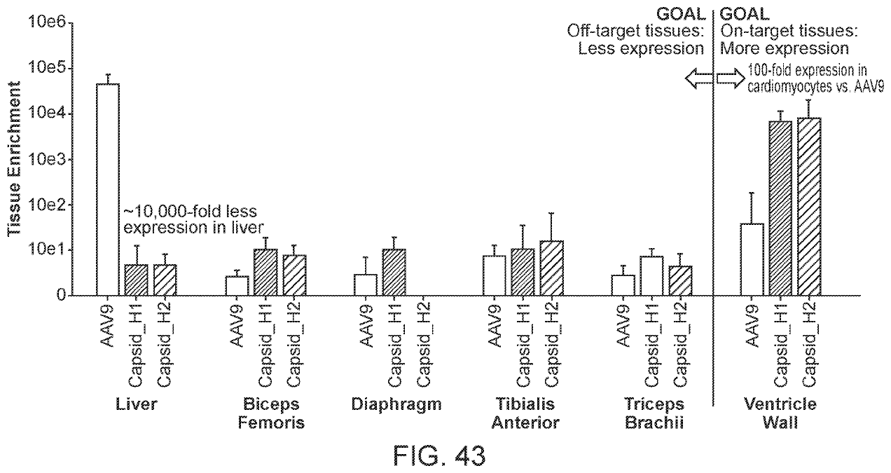

As shown in FIG. 43. SAR analysis identified at least two modified AAV capsid proteins (i.e., modified AAV capsid proteins comprising either targeting peptide H1 (RGDLIGR (SEQ ID NO: 1422)) or targeting peptide H2 (RGDQSTL (SEQ ID NO: 3052)) inserted into VR VIII of AAV9 between amino acids at positions 588 and 589)) that increased tissue enrichment in cardiac muscle tissue as compared to skeletal muscle tissue.

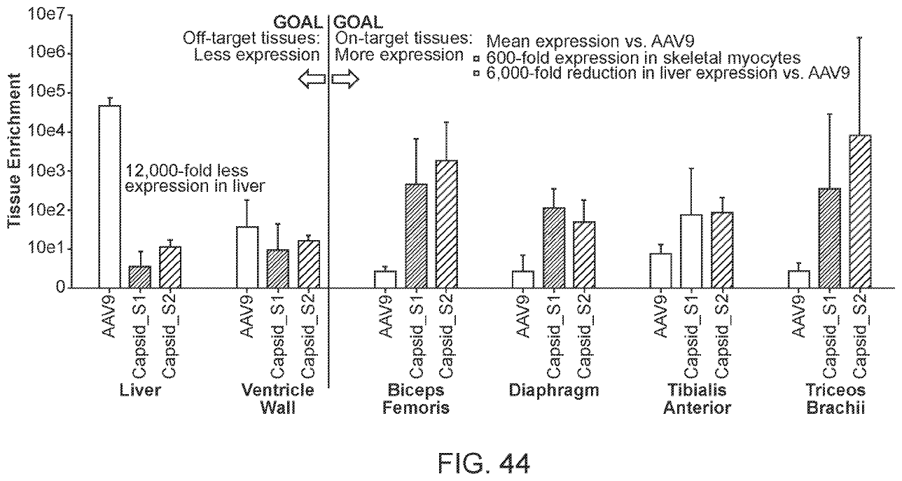

As shown in FIG. 44, SAR analysis also identified at least two modified AAV capsid proteins (i.e., modified AAV capsid proteins comprising either targeting peptide S1 (RGDISRT (SEQ ID NO: 263)) or targeting peptide S2 (RGDRSQT (SEQ ID NO: 251)) inserted into VR VIII of AAV9 between amino acids at positions 588 and 589) that increased tissue enrichment in skeletal tissue as compared to cardiac muscle tissue.

### 8.14. Example 14: Clonal Analysis of a Subset of Modified AAV Capsid Proteins in Non-Human Primates

The objective of this study was a clonal assessment of selected modified AAV capsid proteins comprising different targeting peptides in VR VIII in non-human primates.

rAAVs comprising modified capsid proteins having a targeting peptide having a sequence of SEQ ID NO: 44864 (ENRRGDFNNL) (M3 or MYODV6) or a targeting peptide having a sequence of SEQ ID NO: 44911 (SAQRGDRGQI)) in VR VIII of AAV9 were constructed as described herein (i.e., the targeting peptide was inserted between 585 and 589 such that amino acids at positions 586, 587 and 588 were replaced with three amino acids from the targeting peptide in AAV9 VP1 capsid protein). rAAVs comprising an M1 targeting peptide in VR VIII of AAV9 were constructed as described herein (i.e., the targeting peptide was inserted between 585 and 589 such that amino acids at positions 586, 587 and 588 were replaced with three amino acids from the targeting peptide in AAV9 VP1 capsid protein) and a mut1 VR I were also tested. rAAV comprising capsid proteins having a targeting peptide with the amino acid sequence of SEQ ID NO: 44880 (ENRRGDFNNT) was used as a control. AAV9 with no targeting peptide in VR VIII was also used as a control. The control rAAV and rAAV comprising the modified capsid proteins were administered to the NHPs according to the experimental design in the table 13 below.

TABLE 13
Experimental design for Example 14.

| | | | | | Study end |
|---|---|---|---|---|---|
| | | Dose | Dose | Dose (vg/ | point (necropsy |
| Animals | Treatment | Route | Vol (ml) | animal or kg) | day 28) |
| 1-3 | AAV9 | IV | 10.0 | 5E13 vg/kg | 3 |
| 4-6 | ATLVT019XXAAV9_1000 | IV | 10.0 | 5E13 vg/kg | 3 |
| | (SEQ ID NO: 44880 | | | | |
| | (ENRRGDFNNT)) | | | | |
| 7-9 | ATLVT019XX67 | IV | 10.0 | 5E13 vg/kg | 3 |
| | SEQ ID NO: 44864 | | | | |
| | (ENRRGDFNNL) | | | | |
| | (M1 or MYODV6) | | | | |
| 10-12 | ATLVT013XX38181 | IV | 10.0 | 5E13 vg/kg | 3 |
| | SEQ ID NO: 44911 | | | | |
| | (SAQRGDRGQI) | | | | |
| | (38181) | | | | |
| 13-15 | ATLVT013XX38181 | IV | 10.0 | 5E13 vg/kg | 3 |
| | SEQ ID NO: 44911 | | | | |
| | (SAQRGDRGQI) | | | | |
| | (38181mut1) | | | | |

The NHPs were sacrificed 28 days after the injection. Individual muscle tissues, notably the tibialis anterior, quadriceps, biceps femoris, gastrocnemius, or ventricle wall were collected at the time of necropsy. For RNA, tissues were immediately placed into the preservative RNAlater, after which the RNAlater was removed and the tissue flash frozen. For DNA, tissues were homogenized in a Qiagen Tissuelyser II (20 rps for 2 min) in lysis buffer from the Qiagen Dneasy Blood and Tissue Kit or the Qiagen RNeasy Lipid Tissue Mini Kit following the standard Qiagen protocol. The same tissues were fixed and embedded for sectioning and anti-GFP staining by immunohistochemistry (IHC) as described herein.

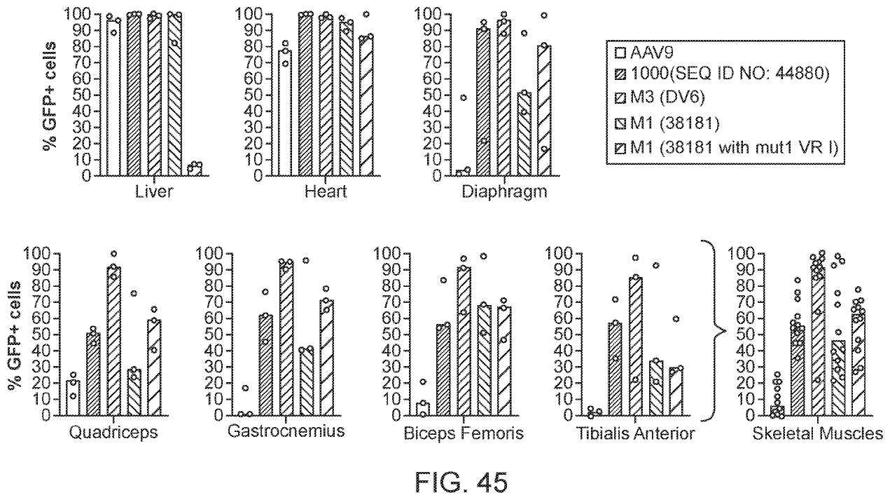

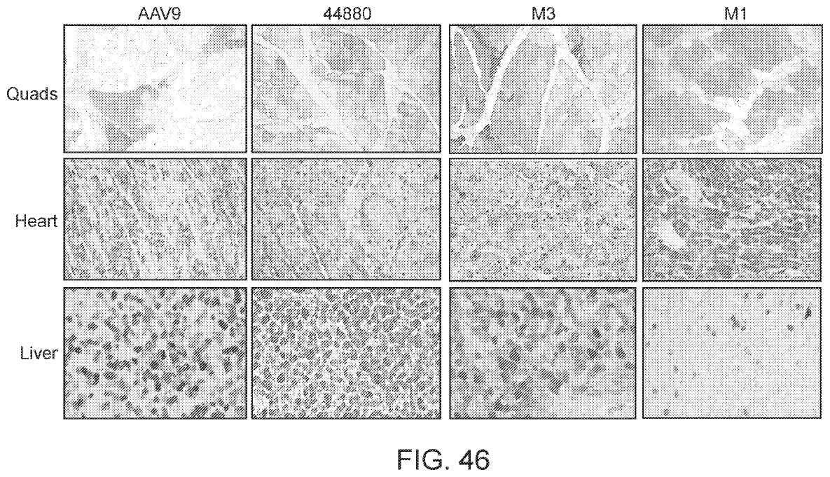

FIG. 45 shows the median percent GFP⁺ cells for liver and the indicated muscle tissues for AAV9 and AAV9 comprising an M3 (“DV6” ENRRGDFNNL (SEQ ID NO: 44864)) targeting peptide located in VR VIII (M3 targeting peptide was inserted between 585 and 589 such that amino acids at positions 586, 587 and 588 were replaced with three amino acids from the targeting peptide in AAV9 VP1 capsid protein); an M1 (“38181” SAQRGDRGQI (SEQ ID NO: 44911)) targeting peptide located in VR VIII (M1 targeting peptide was inserted between 585 and 589 such that amino acids at positions 586, 587 and 588 were replaced with three amino acids from the targeting peptide in AAV9 VP1 capsid protein) and a mut1 VR I substitution, or an M1 (“38181” SAQRGDRGQI (SEQ ID NO: 44911)) targeting peptide located in VR VIII (M1 targeting peptide was inserted between 585 and 589 such that amino acids at positions 586, 587 and 588 were replaced with three amino acids from the targeting peptide in AAV9 VP1 capsid protein) with a wild type VR I. Capsids comprising either the M1 targeting peptide or the M3 targeting peptide also showed increased tissue enrichment (i.e., increased tropism) in each of the muscle tissues analyzed as compared to AAV9 (see FIG. 45). FIG. 46 shows representative images of IHC quantified in FIG. 45.

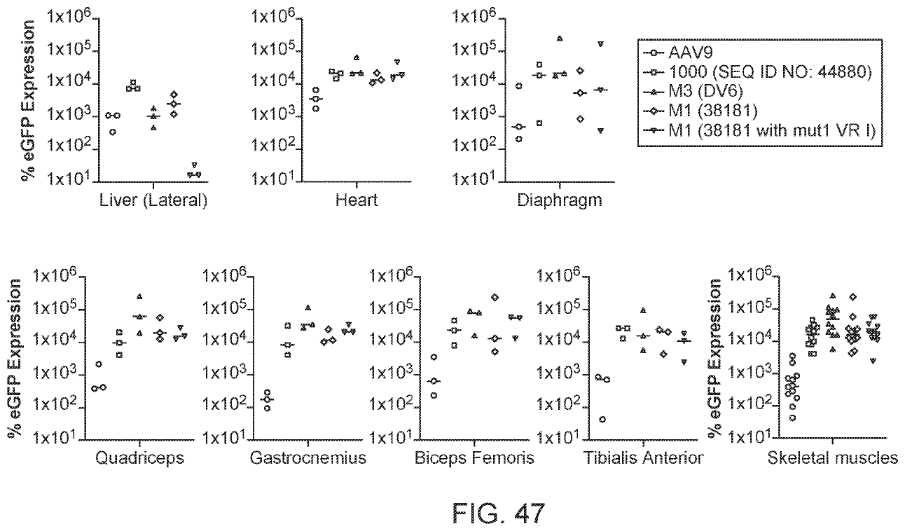

Results for ddRT-PCR shown eGFP RNA copies per copies of NHP RPP30 RNA is shown in the table 14 below) and FIG. 47. ddRT-PCR was performed at Affinia Therapeutics (Waltham, MA).

TABLE 14
ddRT-PCR data.

| | | VR VIII | | | | | | | |
|---|---|---|---|---|---|---|---|---|---|
| | | SEQ ID | | | Tibialis | | Biceps | | Ventricle |
| Variant | VR VIII | NO: | Animal | Diaphragm | Anterior | Quadriceps | Femoris | Gastrocnemius | Wall |
| AAV9 | Wt | — | 1 | 492.1 | 705.1 | 2505.7 | 232.6 | 173.5 | 1924.0 |
| | | | 2 | 8750.7 | 853.8 | 418.8 | 3507.0 | 287.1 | 11831.9 |
| | | | 3 | 208.3 | 42.2 | 380.5 | 641.7 | 92.8 | 4567.5 |
| ATLVT019 | ENRRGDFNNT | 44880 | 4 | 40173.1 | 26599.4 | 20347.2 | 7955.1 | 8242.2 | 17843.3 |
| XXAAV9_1000 | | | 5 | 641.5 | 26642.8 | 9801.2 | 45916.7 | 25697.3 | 10854.0 |
| | | | 6 | 18439.5 | 12852.9 | 4068.8 | 23015.3 | 4089.0 | 33818.0 |
| ATLVT019 | ENRRGDFNNL | 44864 | 7 | 17928.1 | 5638.9 | 19437.9 | 15907.5 | 27087.2 | 16086.6 |
| XX67 | | | 8 | 256249.4 | 97848.5 | 266768.8 | 87402.1 | 115773.9 | 14348.7 |
| (MYODV6) | | | 9 | 21159.9 | 13508.0 | 62078.7 | 89611.3 | 34642.6 | 7992.1 |
| ATLVT013 | SAQRGDRGQI | 44911 | 10 | 25910.0 | 20711.1 | 57160.4 | 235158.3 | 24079.2 | 17205.9 |
| XX38181 | | | 11 | 860.1 | 23940.4 | 19592.8 | 5034.9 | 11440.6 | 7624.6 |
| (38181) | | | 12 | 5371.3 | 4221.9 | 12590.9 | 12922.0 | 9971.5 | 14680.2 |
| ATLVT013 | SAQRGDRGQI | 44911 | 13 | 168927.6 | 10874.6 | 38166.3 | 12894.7 | 20214.1 | 72001.5 |
| XX38181 | | | 14 | 6651.2 | 17956.0 | 16984.7 | 56899.1 | 34666.0 | 5808.5 |
| (38181mut1) | | | 15 | 369.6 | 2415.7 | 12711.1 | 54755.1 | 20692.4 | 11766.4 |

Overall, this data showed that a modified AAV capsid protein comprising a targeting peptide of SEQ ID NO: 44864 and a modified AAV capsid protein comprising a targeting peptide of SEQ ID NO: 48391 each had increased muscle tropism compared to the controls.

### 8.15. Example 15: Analysis of AAV VRI (AAV^{-Lib1}) Capsid Library

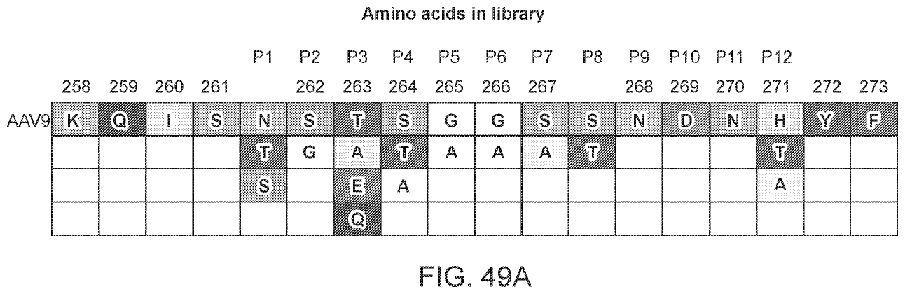

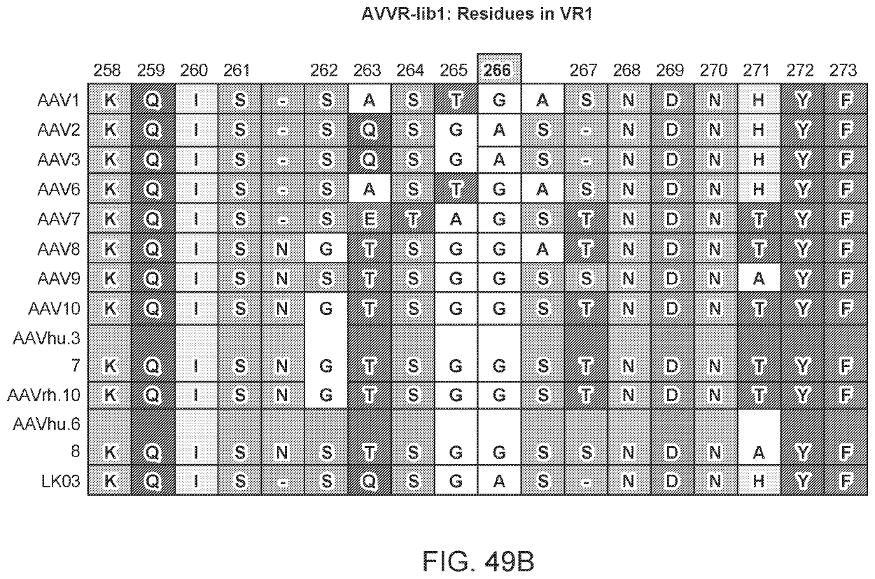

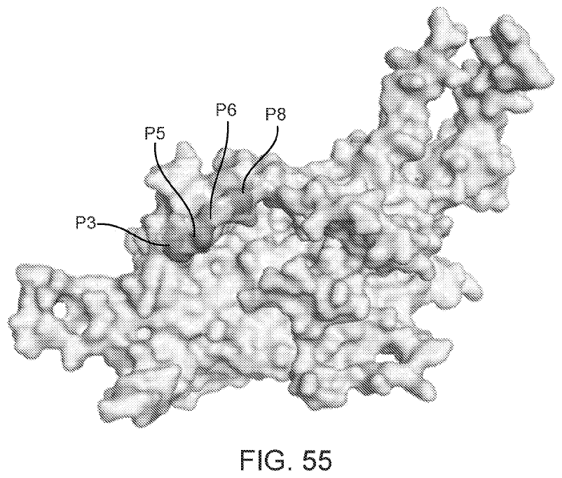

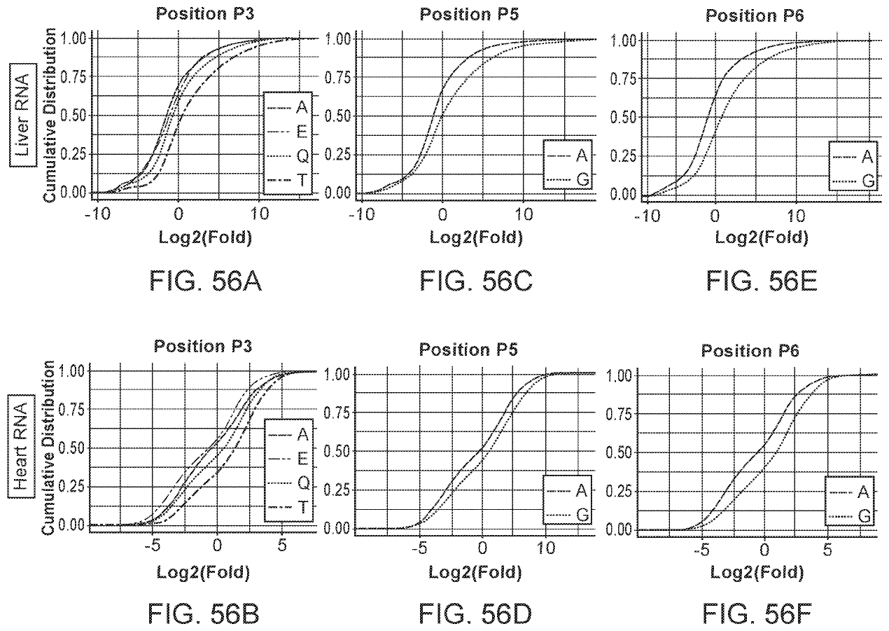

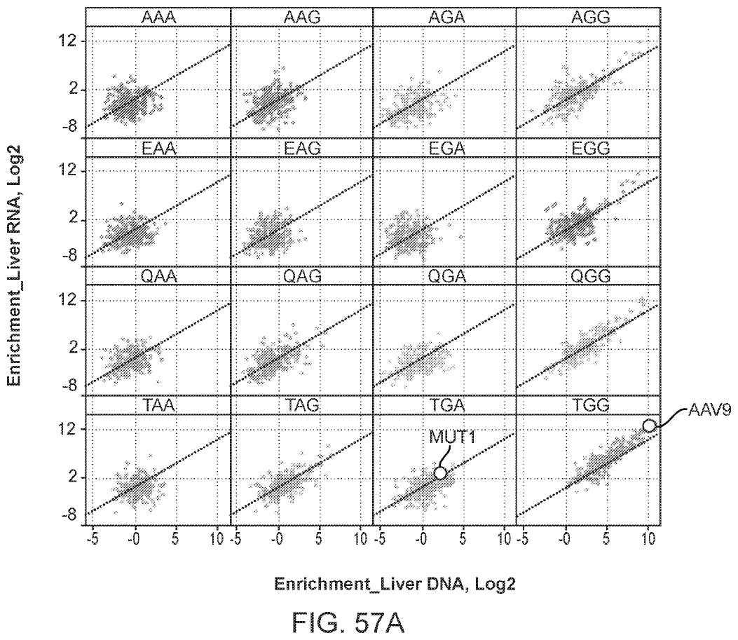

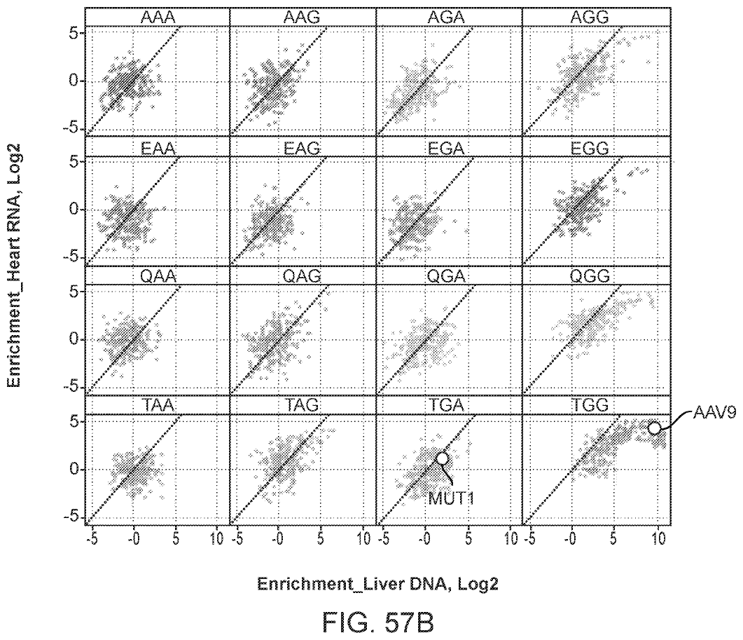

Experiment AFT-MR0026 was designed to identify one or more amino acid modifications in variable region I (VR I) of an AAV capsid protein that decreases liver tropism and increases muscle tropism (or do not effect reduced muscle tropism as compared to a control capsid, such as the reference capsid). Initially, amino acid modifications were made in VR I based, in part, on the idea that amino acid residues in VR I that are important for binding to AAV-R overlap with the residues known to impact liver tropism (e.g., G267A in AAV9, which is the same as GP₆A in the instant application). However, to explore VR I's hidden potential to impact tropism, amino acid modifications were made at amino acid positions throughout the VR I region (e.g., between about position 258 and position 275 in a AAV9 reference capsid protein) (see FIG. 49A). Surprisingly, amino acid modifications made at amino acid residues within VR I known to target the liver (e.g., G267G in AAV9 which is the same as GP₆G in the instant application) resulted in modified capsid proteins having reduced liver tropism and increased muscle tropism (as compared to control capsids). These surprising results were also seen when analyzing modified capsid proteins having combinations of modifications, including, but not limited to, modifications at P₃P₅P₆P₈ that included threonine (T), glycine (G), glycine (G), and threonine (T), respectively (see FIG. 55, FIGS. 56A-56E, and FIGS. 57A-57B). Overall, this experiment proved that one or more amino acid modifications in the VR I region of an AAV capsid protein can enhance muscle tropism while also decreasing liver tropism.

### 8.16. Example 16: Wild Type AAV9 and AAV^{mut1} Tropism in Liver and Muscle in C57BL/6 Mice

As a preliminary assessment for determining whether mice were a suitable system for an initial screen of the AAV-^{Lib1} capsid proteins, an analysis of AAV vector tissue distribution was performed in mice following IV injection of AAV^{mut1} and wild type AAV9. A low dose of 1×10¹³ gc/kg and a high dose of 5×10¹³ go/kg were IV administered for AAV capsid to 10 week old C57BL/6 mice (see Table below). Additionally, a control mouse was injected with vehicle (1×PBS, 35 mM NaCl, 0.001% pluronic) alone. Thus, a total 14 mice comprised the study

TABLE 15
Study design table for assessing mice as
suitable model for screening AAV^{-Lib1}

| | low dose | high dose |
|---|---|---|
| Vector (AAV) | (1 × 10¹³ gc/kg) | (5 × 10¹³ gc/kg) |
| AAV9 CAG.GFP “AAV9” | 3 mice | 3 mice |
| AAVmut1 CAG.GFP “AAV^{mut1}” | 3 mice | 3 mice |

| | |
|---|---|
| Control (vehicle) | 2 mice |

Tissues were harvested 28 days after administration. Gene transfer efficacy of each AAV vector was tested with three C57BL/6 mice, injected with wild type AAV9 or AAV^{mut1} at one of the two different doses by intravenous tail vein injection. Total fourteen mice were injected in total as summarized in the above table. The low dose was 1×10¹³ gc/kg (total 2×10¹¹ gc), and the high dose was 5×10¹³ gc/kg (total 1×10¹² gc).

GFP expression was assessed by ddRT-PCR for the eGFP vector genome copies per DPG (DNA) and transcript (mRNA). The eGFP transcript level was compared against the transcript of a housekeeping standard RPP30. IHC was performed at Histoserv Inc. (Germantown, MD). ddRT-PCR was performed at Affinia Therapeutics (Waltham, MA).

Transgene transfer and expression capabilities of administered vectors were also evaluated with ddPCR, by measuring amounts of DNA and mRNA of the transgene (eGFP) in the various tissue samples 28 days after injection. DNA genome copies and mRNA transcript copies of the transgene (eGFP) were quantified in comparison to the amounts of DNA genome copies or mRNA transcript copies of a house keeping gene (RPP30), respectively. Specifically, DNA genome copies are reported as vector genomes copies per diploid genome (VGC/DG). The formula for calculating the output is VGC/DG=(eGFP cp/μL÷RPP30 cp/μL)×2. RNA transcript copies are reported as % eGFP expression, which is calculated according to the formula, % eGFP expression=(eGFP cp/μL÷RPP30 cp/μL)×100.

Tissues were homogenized in a Qiagen Tissuelyser II (20 rps for 2 min) in lysis buffer from the Qiagen Dneasy Blood and Tissue Kit or the Qiagen RNeasy Lipid Tissue Mini Kit following the standard Qiagen protocol. Samples were eluted in 50 μL of buffer. Prior to analysis, DNA and RNA concentration and quality were determined using a NanoDrop One, using the nucleic acid (DNA or RNA) program. DNA samples were analyzed for biodistribution of vector genomes using a duplexed ddPCR method targeting the transgene (eGFP) and a reference gene (RPP30). RNA samples were analyzed for expression of the eGFP transgene using a duplexed, one-step RT-ddPCR method and a reference gene (RPP30).

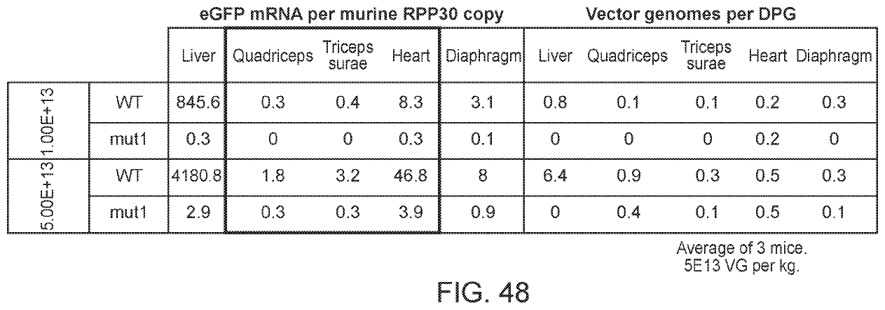

mRNA was extracted from 30 mg sections of liver, and quadriceps. The results of the ddPCR assays are shown in FIG. 48 which show that AAV^{Mut1} has low liver tropism but does not have high muscle tropism, AAV^{Deco1} has high liver tropism and comparatively high muscle tropism, and that AAV^{Mut1_deco1} has decreased liver tropism and increased muscle tropism compared to AAV9 (WT). Other muscle tissues examined, discussed below, and showed a similar trend and the DNA and RNA results generally agree.

eGFP mRNA expression in various tissues was measured by RT-ddPCR and presented as the ratio of eGFP transcripts over RPP30 transcripts, a rough indicator of eGFP mRNA copies per cell. The results are provided in FIG. 48 for liver, heart, tricep surae, quadricep, and diaphragm. For each tissue, results from three biological replicates are provided for each AAV variant at each dose (high or low dose).

FIG. 48 provides the eGFP mRNA per murineRPP30 transcript in the liver, quadriceps, triceps surae, heart, and diaphragm. For each dose group, mice treated with AAV^{mut1} had lower eGFP expression compared to mice treated with AAV9 for each of the five tissues measured (see FIG. 48). FIG. 48 also provides the vector genomes per DPG in the liver, quadriceps, triceps surae, heart, and diaphragm. For liver, quadriceps, triceps surae, and diaphragm, mice treated with AAV^{mut1} had fewer vector genomes per DPG compared to mice treated with AAV9 for ease dose group (see FIG. 48). For heart, AAV^{mut1} and AAV9 showed similar vector genomes per DPG for each dose group (FIG. 48).

### 8.17. Example 17: AAV VRI (AAV^{-Lib1}) Capsid Libraries

The polynucleotide encoding the wild-type AAV9 VP1 capsid protein (SEQ ID NO: 61) was modified by insertion, deletion or substitution of one or more amino acid residues between S261 and Y274 in the VR I region of AAV9. The amino acid positions between S261 and Y274 of AAV9 capsid are represented as “P₁, P₂, P₃, P₄, P₅, P₆, P₇, P₈, P₉, P₁₀, P₁₁, and P₁₂” as illustrated in FIG. 49A. In total, a library containing 3,456 unique sequences of modified AAV9 VP1 capsid proteins was produced, which is referred to herein as the AAV^{-Lib1} library or AAV^{-Lib1} capsid library. The amino acid sequences (a peptide segment in the VR I region) corresponding to the 12 amino acids between S261 and Y274 for each of the 3,456 sequences are as described in SEQ ID NOs: 251-3703. The amino acid modifications (insertion, deletion, substitution) in the VR I region of AAV9 include those described herein as shown in FIG. 49A. For reference, a sequence alignment of variable region I for certain AAV variants is shown in FIG. 49B.

### Library Construct Design and Capsid Library Preparation

The construct used for generating each of the AAVs for the AAV library (AAV^{-Lib1}) included a polynucleotide sequence located between the ITRs that encoded a sequence that was reflective of the genome of the virus that transduced the cell. For example, the AAV9 control construct included a polynucleotide sequence encoding the wild type AAV9 capsid protein (e.g., SEQ ID NO: 61). This polynucleotide was then encapsulated in an AAV virion that included an AAV9 capsid protein. For the AAV^{mut1} control, the construct included a polynucleotide sequence encoding the AAV^{mut1} capsid protein (e.g., SEQ ID NO: 163). This polynucleotide was then encapsulated in an AAV virion that included an AAV^{mut1} capsid protein. For each of the AAVs in the AAV^{-Lib1} library, each construct included polynucleotide sequence encoding a modified AAV capsid protein. This polynucleotide was then encapsulated in an AAV virion that included the same modified AAV capsid protein.

An AAV library was generated by transfecting the pooled plasmid library into HEK293 cells together with an adenoviral helper gene plasmid construct and a plasmid containing the AAV rep expression cassette. After AAV production, the AAV library was assessed for its diversity using sequencing (i.e., next-generation sequencing using Illumina reagents and hardware). DNA was isolated from viral supernatant and assessed for the presence of each of the 3,456 modified capsids based on the presence of the polynucleotide sequence encoding the same modified capsid variant present in the AAV virion. This data provided a count for each polynucleotide sequence encoding the same modified capsid variant present on the AAV virion by the sequencing, and thereby, gave a relative representation of the capsid variant using the barcode as a proxy. NOS sequencing data of the AAV library indicated that the AAV^{-VRI} library included each the 3,456 capsids as expected and included an equal distribution of the 3,456 capsid variants. As noted above, wild-type AAV9 and AAV^{mut1} capsid proteins were used as controls.

The transduction process by AAVs and related vectors resulted in both the presence of DNA and the production of an mRNA molecule both of which were reflective of the genome of the virus that transduced the cell. As demonstrated herein, in some embodiments, mRNA based-detection of a modified capsid protein using NGS was more specific and effective to determine a virus particle capable of functionally transducing a cell because it was based on the functional product produced from the AAV genome. Viral DNA using NOS was also used to detect the presence of a virus particle in the cell.

### Experimental Design

Experimental designed for testing the AAV^{-Lib1} library is as described in the Table 16 below. Group I included a control mouse injected with vehicle (1×PBS, 35 mM NaCl, 0.001% pluronic) alone. For Group 2, 5×10¹³ gc/kg of AAV^{-Lib1} Library was administered intravenously to each 10 week old C57BL/6 mice.

TABLE 16
Study designed for AAV^{-Lib1} capsid library.

| | | | | | | | Post |
|---|---|---|---|---|---|---|---|
| Group | No. of | Route of | | | | | Procedure |
| No. | Animals | Administration | Strain | Treatment | Dosage | Dosage | Analysis |
| 1 | 1 | IV | C57BL/6 | Vehicle | NA | vg/kg | 28 days |
| 2 | 8 | IV | C57BL/6 | AAV^{-Lib1} Library | 5E13 | vg/kg | 28 days |

### Tissue Analysis

Successful gene transfer by AAV^{-Lib1} Library (and controls) was detected by NGS sequencing of DNA and/or RNA harvested from mouse tissue following IV injection of the AAV^{-Lib1} library. In particular, the mice were sacrificed 28 days after the injection. Individual tissues, notably the liver, major skeletal muscles of the hind limb, heart, and diaphragm, were collected at the time of necropsy. For DNA, tissues were homogenized in a Qiagen Tissuelyser II (20 rps for 2 min) in lysis buffer from the Qiagen Dneasy Blood and Tissue Kit or the Qiagen RNeasy Lipid Tissue Mini Kit following the standard Qiagen protocol. Samples were eluted in 50 μL of buffer. For RNA, tissues were immediately placed into the preservative RNAlater, after which the RNAlater was removed and the tissue flash frozen. Variations to these general protocols are as described below.

### AAV^{-Lib1} Library Screen Data Informatics Analysis

Next generation sequencing (NGS) was used to determine the presence of AAV vector genomes (i.e., DNA) and to determine expression of AAV components (i.e., viral RNA) such as capsid sequences encoded by the AAV vector DNA. Following DNA and/or RNA isolation, sequencing library preparation, and sequencing, the resulting FASTQ reads were subjected to SeqCount QC (i.e., quality control of sequencing reads) before determining Variant Counts. Variant Counts represent the total Variant Count (i.e., sequencing reads associated with a particular capsid variant) in a sample or input test article. Prior to determining tissue enrichment, Variant Count data was normalized. Normalization included two steps. Step 1 included sequencing depth normalization:

$$log 2 CPM = log 2 ( \frac{RawVariantCount + 0.5}{TotalVariantCount + 1} \times 1 , 000 , 000 )$$

Step 2 included distribution normalization by median:

$$log 2 MN = log 2 CPM - median ( log 2 CPM )$$

Tissue enrichment score was calculated using:

$$log 2 FC = log 2 {MN}_{Tissue} - log 2 {MN}_{TestArticle}$$

$$scaled log 2 FC = \frac{( log 2 FC - min ( log 2 FC ) )}{( max ( log 2 FC ) - min ( log 2 FC ) )}$$

Tissue enrichment scores for all 3,456 variants and controls are as shown in Table 28, which is herein incorporated by reference in its entirety. The tissue enrichment measurements were used for functional interpretation of the data as well as for variant ranking and selecting capsids from the AAV^{-Lib1} library. As shown in the following Examples, tissue enrichment analysis identified numerous peptides segments having the desired tropism (e.g., decreased liver tropism and increased muscle tropism (or do not effect reduced muscle tropism as compared to a control capsid, such as the reference capsid (e.g., AAV9)). The tissue enrichment scores were also used for sequence activity relationship (SAR) analysis, network analysis, and structural modeling.

Sequence activity relationship (SAR) analysis of the AAV^{-Lib1} data was used to identify particular amino acid residues at specific amino acid positions and/or peptide segments (i.e., combinations of specific amino acid residues at specific amino acid positions) within VRI of the AAV capsid protein that significantly impact tissue tropism. Tissue enrichment scores from Table 28 were used for SAR analysis. Example 13 provides an example SAR analysis performed using the AAV^{-Lib1} data that identified particular amino acid residues at specific amino acid position and/or peptide segments within VRI of the AAV capsid proteins that significantly impacted tissue tropism (see, e.g., peptide segments 1-5 corresponding to SEQ ID NO: 44921-44926).

Network analysis of the AAV^{-Lib1} data was used to identify AAV capsid variants (from the 3,456 capsids of AAV^{-Lib1}) that shared amino acid residues at specific amino acid positions and/or peptide segments within VRI based. Tissue enrichment scores from Table 28 were used for the network analysis. For example, network analysis was used to identify the peptide segments (i.e., amino acid sequences) within VRI for the top performing capsids from the AAV^{-Lib1} library data (i.e., the AAV^{-Lib1} tissue enrichment data provided in Table 28). This analysis helped identify AAV capsids from the AAV^{-Lib1} data that significantly impacted tissue tropism and had similar peptide segments in VRI, including clusters 1-7 corresponding to SEQ ID NOs: 44927-44933.

Structural modeling was used to study the structural impact of particular amino acid residues at specific amino acid positions within the AAV^{-Lib1} capsids. Tissue enrichment scores from Table 28 were used for structural modeling. Results of structural modeling were used for mechanistic hypothesis formulation. In one example, Example 14 illustrates how structural modeling helped informed the hypothesis that particular amino acid residues at P₃, P₅, P₆, and P₈ interacted with AAV receptor (AAVR), and therefore, were key to tissue tropism.

Overall, tissue enrichment scores were used along with SAR analysis, network analysis, and structural modeling to interpret the data generated by the AAV^{-Lib1} library (e.g., data provided in Table 28). The Examples below show how analysis of tissue enrichment scores, SAR analysis, network analysis, and structural modeling were used to identify particular amino acid residues at particular amino acid positions that impact tropism.

### 8.18. Example 18: Analysis of AAV-Lil Data Showed Capsid Variants with Enhanced Muscle Tropism with Limited Liver Tropism in C57BL/6 Mice

For analysis of the AAV^{-Lib1} library. NGS was performed on DNA and/or RNA harvested from mouse tissues 28 days after IV injection of the AAV^{-Lib1} library according to the methods described herein.

In particular, gene transfer efficacy of each AAV within the AAV^{-Lib1} library was analyzed in liver and muscle (quadriceps) harvested 28 days after administration. DNA and RNA were isolated as described above and NGS was performed to determine levels of expression (RNA) and vector genomes (DNA) present in liver and muscle. Data (i.e., AAV^{-Lib1} library data) for all 3.456 capsids and controls (AAV^{mut1} and AAV9) is found in Table 28, which is herein incorporated by reference in its entirety.

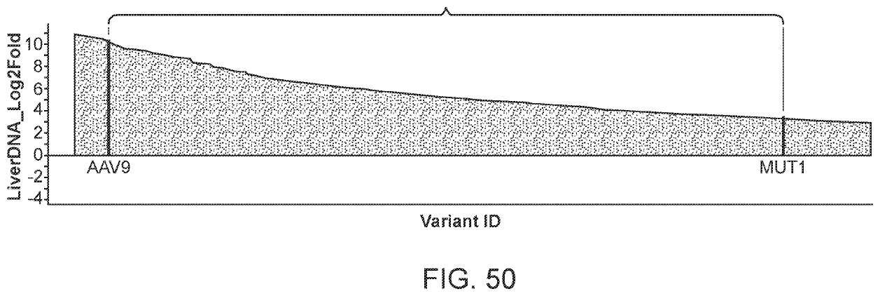

FIG. 50 shows DNA levels of each capsid variant from the AAV^{-Lib1} library (variant ID on x-axis) in the liver represented as Log 2 Fold Change over test article (see AAV^{-Lib1} Library Screen Data Informatics Analysis in Example 11 above for Log 2Fold Change calculation). As noted above, the AAV^{-Lib1} library included AAV^{mut1} and wild type AAV9 as controls. As expected, AAV^{mut1} had lower liver tropism than wild type AAV9 (as described in PCT Application No. PCT/US2022/015842, which is herein incorporated by reference in its entirety). Additionally, FIG. 50 shows the AAV^{-Lib1} library includes modified capsid proteins having a range of liver tropic capsid variants. Overall, this data showed that the AAV^{-Lib1} library includes modified capsids having one or more amino acid modifications in VR I that decrease liver tropism.

As noted above, Applicant hypothesized that one or more of these modified capsid proteins would not only reduce liver tropism but also potentially increase muscle tropism (or do not effect reduced muscle tropism as compared to a control capsid, such as the reference capsid). The following examples provide a summary of the analysis performed on data from the AAV^{-Lib1} library study aiming to identify modified capsid proteins having the properties of interest.

### 8.19. Example 19: Sequence Activity Relationship (SAR) Analysis of AAV^{-Lib1}

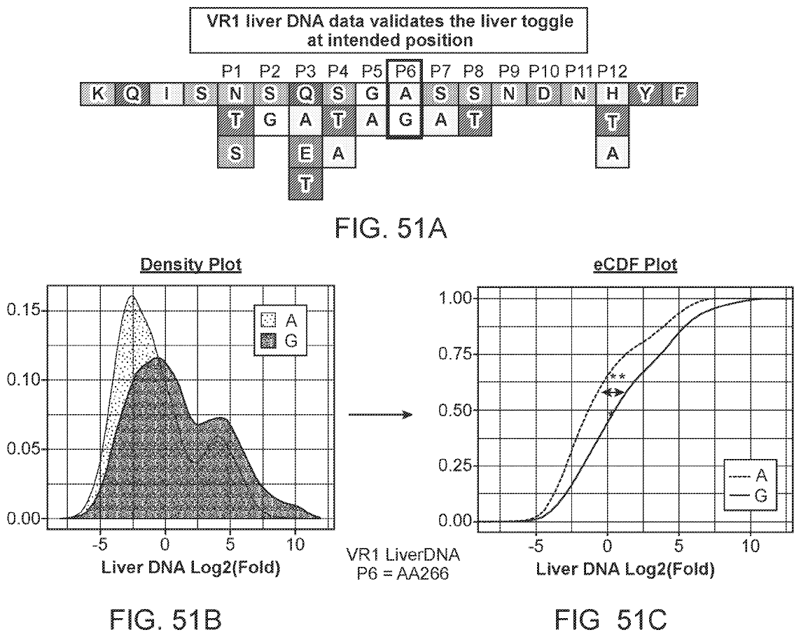

Sequence activity relationships (SARs) of the data from the AAV^{-Lib1} library were used to determine which amino acid modifications at which residues (or combinations of amino acid modifications at various residues) in VRI decrease liver tropism. As one example of SAR, capsids with the AAV^{-Lib1} library having an amino acid substitution at position P₆ (amino acid position 266 in AAV9) were analyzed for impact on liver tropism. The AAV^{-Lib1} library includes capsid having an alanine (A) or a glycine (G) at amino acid position P₆ (FIG. 51A). This includes the alanine (A) found in the control AAV^{mut1} capsid (e.g., G266A) and the guanine (G) found in AAV9 (e.g., G266). Twenty-eight days after injection. DNA was isolated from mouse liver according to the methods described herein and sequenced to determine the amount of DNA from each capsid variant from AAV^{-Lib1} present in the liver. As shown in FIG. 51B (density plot) and FIG. 51C (empirical cumulative distribution function), an alanine (A) at P₆ (amino acid position 266) resulted in less AAV DNA in the liver than compared to when a glycine (G) was present at P₆ (amino acid position 266). This data showed that SAR could be used to identify amino acid residues in VRI that reduce liver tropism—an integral step in optimizing capsid amino acid sequences for suitability as a gene therapy vector.

Next, SAR analysis was used to determine which amino acid modifications at positions P₁, P₂, P₃, P₄, P₅, P₆, P₇, P₈, and P₁₂ decrease liver tropism (see FIG. 52A).

In particular, amino acid modifications at positions P₃, P₅, and P₆ induced statistically significant changes in liver tropism. Amino acid modifications that were tested at P₃ included an alanine (A), a glutamate (E), a glutamine (Q), and a threonine (T). As shown in FIG. 52B, modification to an alanine (A) or a glutamate (E) at P₃ reduce liver tropism as compared to capsids having a glutamine (Q) or a threonine (T) at P₃. Amino acid modifications that were tested at P₅ included an alanine (A) and a glycine (G). As shown in FIG. 52C, modification to an alanine (A) at P₅ reduced liver tropism compared to capsids having a glycine (G) at P₅. Amino acid modifications that were tested at P₆ included alanine (A) and glycine (G). As shown in FIG. 52D, modification to an alanine (A) at P₆ reduced liver tropism compared to capsids having a glycine (G) at P₆. Overall, this data that shows that amino acid modifications at individual positions P₃, P₅, and P₆ can each alone reduce liver tropism.

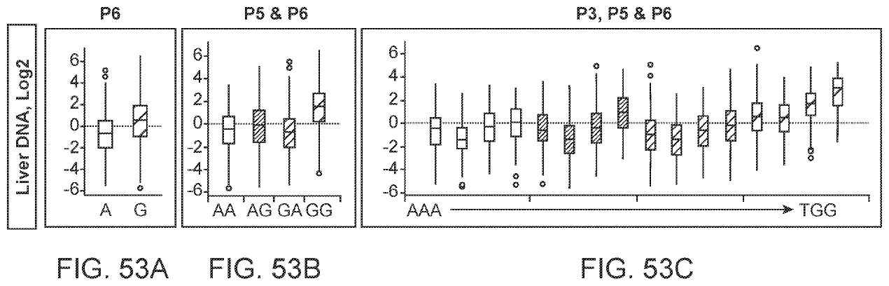

Next, Applicant hypothesized that amino acid substitutions at positions P₃, P₅, and P₆ could be combinatorial in the impact on liver tropism. Capsids comprising various amino acid residues at P₆ (FIG. 53A), P₅ and P₆ (FIG. 53B), and P₃, P₅, and P₆ (FIG. 53C) were assessed for liver tropism. FIG. 53A confirms previous SAR analysis showing that an analysis of all capsids in AAV^{-Lib1} having an alanine (A) at P⁶ decreased liver tropism (compared to capsids having a glycine (G) at P₆). FIG. 53B illustrates how different combinations of amino acid substitutions at P₅ and P₆ impact liver tropism. An analysis of all capsids in the AAV^{-Lib1} library having a P₅P₆ combination of AA, AG, and GA showed that these combinations reduced liver tropism compared to a P₅P₆ combination of GG. Lastly, combinations of amino acid substitutions at P₃, P₅, and P₆ were tested for impact on liver tropism. FIG. 53C provides an analysis of all capsids in the AAV^{-Lib1} library having a P₃, P₅, and P₆ combination of: AAA, AAG, AGA, AGG, EAA, EAG, EGA, EGG, QAA, QAG, QGA, QGG, TAA, TAG, TGA, and TGG (appearing from left to right on the x-axis in FIG. 53C).

Taken together, this Example showed that sequence activity relationship (SAR) analysis of the AAV^{-Lib1} library data could be used to determine which amino acid modifications at which residues or which peptide segments in VRI impact liver tropism. As the overall aim of the AAV^{-Lib1} library study was to identify AAV capsid proteins that decrease liver tropism and increase muscle tropism (or at least have comparable muscle tropism to a control (e.g., AAV9)), additional analysis (see Examples 20, 21 and 22) was performed to determine which peptide segments could produce the desired tropism.

### 8.20. Example 20: Network Analysis and Structural Modeling of AAV^{-Lib1} Library Data Identified Positions P₃, P₅, and P₆ as Important for Liver Tropism

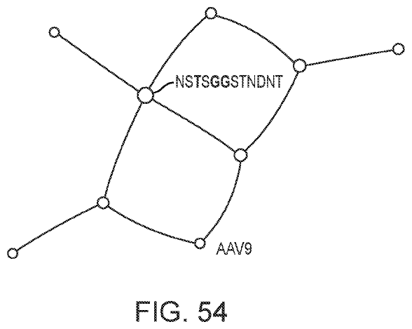

Network analysis of AAV^{-Lib1} library data identified P₃, P₅, and P₆ as important for liver tropism. In particular, network analysis revealed that modified capsid proteins having a VRI region having a threonine (T) at P₃, a glycine (G) at P₅, and a glycine (G) at P₆ were enriched in capsid variants from the AAV^{-Lib1} library that exhibited greater liver tropism than AAV9. For example, as shown in FIG. 54 where each circle indicates a capsid variant and a line connecting the capsids indicates sequence similarity, network analysis revealed, for example, that NSTSGGSTNDNT (SEQ ID NO: 1351) had greater liver tropism than AAV9.

The network analysis supported the structural modeling data in FIG. 55. As shown in FIG. 55, P₃, P₅, P₆, and P₈ correspond to amino acid residues TGGT, respectively, where modelling predicted that P₃T is involved in internal stabilization, P₅G and P₆G maintain loop flexibility, and P₇ and P₈ form direct Hydrogen bonds with AAV receptor (AAVR), where a P₈T resulted in 2 more Hydrogen bonds than other amino acids at P₈.

Overall, network analysis and structural modeling of the AAV^{-Lib1} library data provided an independent means by which to identify amino acid modifications within VRI that impact liver tropism. As noted above, the overall aim of the AAV^{-Lib1} library study was to identify AAV capsid proteins that decrease liver tropism and increase muscle tropism (or do not effect reduced muscle tropism as compared to a control capsid, such as the reference capsid (e.g., AAV9))). Therefore, additional analysis (see Examples 21 and 22) was performed to determine which peptide segments harbored the desired tropism.

### 8.21. Example 21: Analysis of RNA Levels from AAV^{-Lib1} Showed Enrichment Variation in Liver and Heart Tropism Based on Amino Acid Modification P₃, P₅, and P₆

With one of the aims of the AAV^{-Lib1} library study to identify AAV capsid proteins that decrease liver tropism and increase muscle tropism (or at least have comparable muscle tropism to a control (e.g., AAV9)), expression data from mouse heart and mouse liver were analyzed to identify peptide segments that harbored the desired tropism.

For expression analysis, NGS was performed on RNA harvested from mouse tissue following IV infusion of the AAV^{-Lib1} library. As noted above, individual tissues (i.e., the liver and major skeletal muscles of the hind limb (quad)), were collected at the time of necropsy. Tissues were immediately placed into the preservative RNAlater, after which the RNAlater was removed and the tissue flash frozen. As shown in FIGS. 56A-56F, expression analysis of RNA isolated from liver and heart revealed that particular AAV capsids from the AAV^{-Lib1} library exhibited residue-specific variation in tropism based on the particular amino acid modifications at P₃, P₅, and/or P₆. In some instances, the amino acid residues that showed increased muscle tropism also exhibited liver tropism. This suggested that the AAV^{-Lib1} library includes capsids having a range of both muscle tropism and liver tropism.

### 8.22. Example 22: Sequence Activity Relationship (SAR) Analysis Comparing Heart and Liver Data for Amino Acid Modifications at P₃, P₅, and P₆ Showed Specific Amino Acid Combinations Enhanced Muscle Tropism but Reduced Liver Tropism

In this Example, Applicant hypothesized that capsids having particular amino acid residues at specific amino acid positions within VRI harbor both increased muscle tropism and reduced liver tropism and that by modifying the amino acid sequences surrounding these positions, the Applicant could create an AAV capsid that maximizes muscle tropism while also limiting liver tropism. Sequence activity relationship (SAR) analysis based on DNA and RNA isolated from the AAV^{-Lib1} library and analyzed according to the methods described above, revealed that specific combinations of amino acid modifications enhanced muscle tropism and reduced liver tropism. FIG. 57A shows enrichment in liver DNA (x-axis) plotted against enrichment in liver RNA (y-axis) for capsids comprising various combination of amino acid modifications at P₃, P₅, and P₆. As shown in FIG. 57A, some sequence combinations, such as TGG, provide a range of liver detargeting (i.e., capsids having a TGG at positions P₃, P₅, and P₆, respectively) whereas most other sequence combinations detarget the liver. FIG. 57B shows enrichment in liver DNA (x-axis) plotted against enrichment in heart RNA (y-axis) for capsids comprising various combination of amino acid modifications at P₃, P₅, and P₆. As shown in FIG. 57B, some sequence combinations, such as those including QGG and TGG were enriched in both liver and muscle. In contrast, some sequence combinations, such as AAA, AAG, AGA, QAA, QAG, QGA, TAA, TAG, and TGA, had enhanced liver detargeting and increased muscle tropism.

Looking closer at the data for capsids comprising peptide segments having the amino acid combination of a threonine (T), a glycine (G), and a glycine (G) (TGG) at P₃, P₅, and P₆ revealed that this combination at least partially uncoupled liver and heart expression. In other words, expression in the liver did not correlate as tightly with expression in the muscle for capsids comprising the amino acid combination of TGG at P₃, P₅, and P₆.

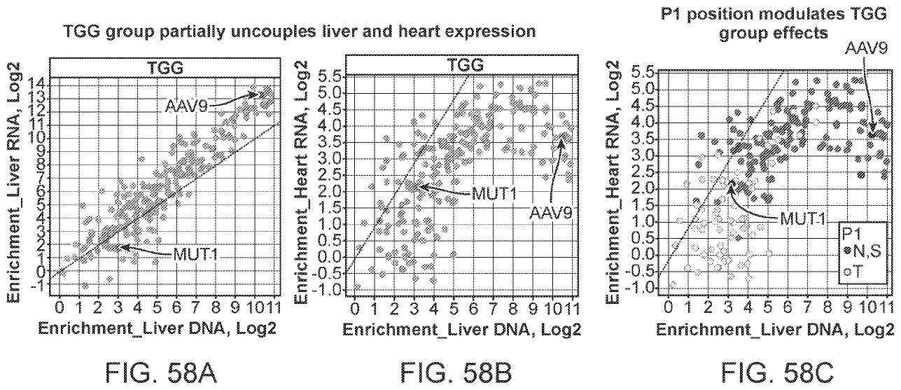

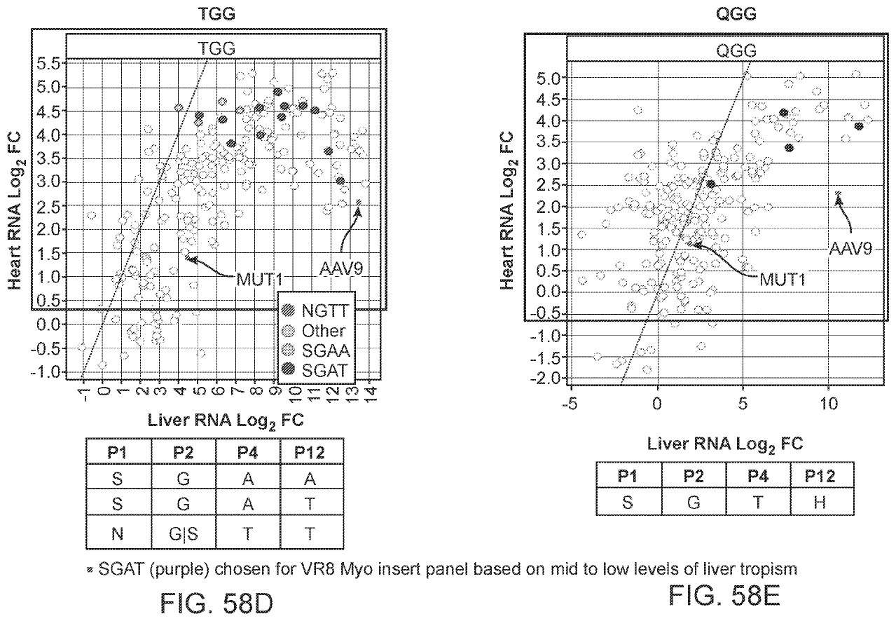

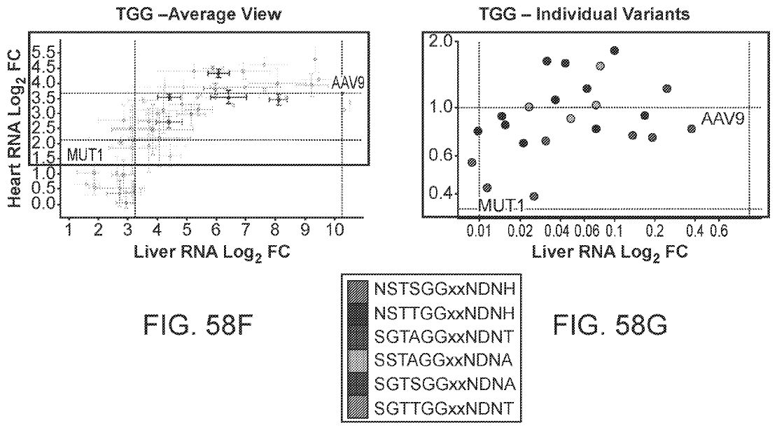

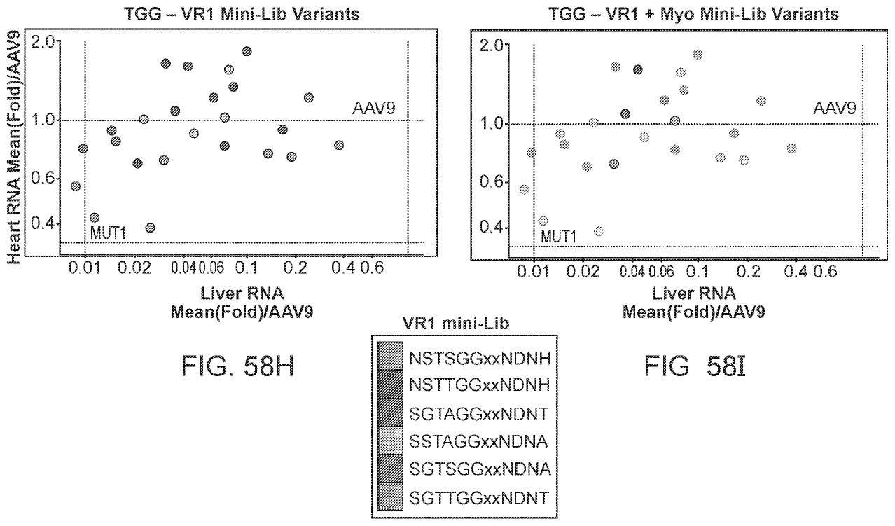

FIG. 58A shows enrichment in liver DNA (x-axis) plotted against enrichment in liver RNA (y-axis) for AAV capsids having a TGG combination of amino acid modifications at P₃, P₅, and P₆, respectively. Overlaying the data for AAV^{mut1} and AAV9 onto the plot in FIG. 58A shows AAV^{mut1} liver tropism compared to AAV9 liver tropism and the liver tropism for a subset capsid variants in the AAV^{-Lib1} library.

FIG. 58B shows enrichment in liver DNA (x-axis) plotted against enrichment in heart RNA (y-axis) for AAV capsids having a threonine (T), a glycine (G), and a glycine (G) at P₃, P₅, and P₆, respectively. Overlaying data for AAV^{mut1} and AAV9 onto the TGG plot in FIG. 58B highlights the lack of correlation between liver RNA and heart RNA for the modified capsids comprising TGG at P₃, P₅, and P₆.

Next, looking for mechanistic insight into the tropism for modified capsids comprising a TGG at P₃, P₅, and P₆, amino acid positions (e.g. P₁) within VRI were analyzed for correlation with the TGG tropism data (see FIG. 58C). This analysis revealed that particular amino acid modifications at the P₁ position correlated with the TOG group impact on tropism, meaning certain modification at P₁ correlated with liver tropism, reduced liver tropism, muscle tropisms, reduce muscle tropism, or a combination thereof. In particular, modified capsid proteins having a threonine (T) at P₁ exhibited heart enrichment levels below that of the control AAV^{mut1} (FIG. 58C, yellow dots). In contrast, modified capsid proteins having an asparagine (N) or a serine (S) at P₁ exhibited increased heart and increased liver enrichment compared to AAV^{mut1} (FIG. 58C, blue dots).

FIG. 58D shows enrichment in liver RNA (x-axis) plotted against enrichment in heart RNA (y-axis) for capsids having a threonine (T), a glycine (G), and a glycine (G) combination of amino acid residues at P₃, P₅, and P⁶, respectively. Particular subpopulations having NGTT, NSTT, SGAA, and SGAT at positions P₁, P₂, P₄, and P¹² are identified. Capsids comprising a peptide segment having a TGG at P₃, P⁵, and P₆ and SGAT at positions P₁, P₂, P₄, and P₁₂ were identified as capsids of interest based on mid to low levels of liver tropism (compared to AAV9) and increased muscle tropism (compared to AAV9). Positions of AAV^{mut1} and AAV9 controls are indicated by arrows and corresponding text.

FIG. 58E shows enrichment in liver RNA (x-axis) plotted against enrichment in heart RNA (y-axis) for capsids having a threonine (Q), a glycine (G), and a glycine (G) combination of amino acid residues at P₃, P₅, and P₆, respectively. Particular subpopulations having SGTH at positions P₁, P₂, P₄, and P₁₂ are identified. Capsids comprising a peptide segment having a TGG at P₃, P₅, and P₆ and SGTH at positions P₁, P₂, P₄, and P₁₂ were identified as capsids of interest based on mid to low levels of liver tropism (compared to AAV9) and increased muscle tropism (compared to AAV9). Positions of AAV^{mut1} and AAV9 controls are indicated by arrows and corresponding text.

FIG. 58F shows average enrichment in liver RNA (x-axis) plotted against enrichment in heart RNA (y-axis) for all AAV^{-Lib1} capsids having a threonine (T), a glycine (G), and a glycine (G) combination of amino acid residues at P₃, P₅, and P₆, respectively. Positions of AAVmut1 and AAV9 controls are indicated by corresponding text.

FIG. 58G shows enrichment in liver RNA (x-axis) plotted against enrichment in heart RNA (y-axis) for 24 selected capsids having a threonine (T), a glycine (G), and a glycine (G) combination of amino acid residues at P₃, P₅, and P₆, respectively. The 24 selected capsids correspond to groups: 1 NSTSGGP₇P₈NDNH (e.g., SEQ ID NO: 44927); group 2: NSTTGGP₇P₈NDNH (SEQ ID NO: 44928); group 4: SGTAGGP₇P₈NDNT (SEQ ID NO: 44930); group 5: SGTSGGP₇P₈NDNA (SEQ ID NO: 44931); group 6: SGTTGGP₇P₈NDNT (SEQ ID NO: 44932); and group 7: SSTAGGP₇P₈NDNA (SEQ ID NO: 44933). FIG. 58G shows that capsids comprising peptides segments sequences in groups 1 to 7 (i.e., SEQ ID NOs: 44927-44933) have detarget the liver while not effecting significant reduction muscle tropism. For example, 3× reduction in muscle (e.g., heart) tropism corresponds to 100× detargeting in the liver. FIG. 58H and FIG. 58I shows enrichment in liver RNA (x-axis) plotted against enrichment in heart RNA (y-axis) for 6 groups where each group has a threonine (T), a glycine (G), and a glycine (G) combination of amino acid residues at P₃, P₅, and P₆, respectively.

Overall, this data showed that AAV capsids comprising peptide segments having sequences in groups 1 to 7 (i.e., SEQ ID NOs: 44927-44933) include capsids having the desired tropism.

### 8.23. Example 23: Summary of Mouse Data

Overall, the AAV^{-Lib1} library data showed that AAV capsids comprising particular peptide segments in variable region I (VRI) bad decreased liver tropism and increased muscle tropism (or at least had comparable muscle tropism to the AAV9 control). For example, Table 17 lists data for 28 AAV capsids selected from the AAV^{-Lib1} library data (see Table 28) with each AAV capsid having a different peptide segment within VRI. These 28 AAV capsids met the criteria of having reduced liver tropism and had increased muscle tropism (or do not effect reduced muscle tropism as compared to a control capsid, such as the reference capsid (e.g., AAV9)). The Liver DNA, Liver RNA. Heart DNA, and Heart RNA values in Table 10 and Table 28 are tissue enrichment scores shown as Log 2Fold Change (FC) to test article.

TABLE 17
Tissue enrichment data for AAV capsids from AAV^{-Libl}
Library comprising the indicated peptide segment in VRI

| | SEQ | Sequence | | | | |
|---|---|---|---|---|---|---|
| | ID | (peptide | Liver | Liver | Heart | Heart |
| variant_id | NO: | segment) | DNA | RNA | DNA | RNA |
| ATLVT012XX1092 | 46026 | NSTSGGASNDNH | 7.98 | 9.47 | 1.07 | 3.41 |
| ATLVT012XX1095 | 46029 | NSTSGGATNDNH | 8.8 | 10.51 | 1.1 | 4.12 |
| ATLVT012XX1097 | 46031 | NSTSGGSSNDNH | 8.46 | 9.88 | 1.13 | 3.37 |
| ATLVT012XX1100 | 46034 | NSTSGGSTNDNH | 9.42 | 10.77 | 0.79 | 3.51 |
| ATLVT012XX1139 | 46073 | NSTTGGASNDNH | 5.29 | 6.39 | 0.68 | 3.29 |
| ATLVT012XX1142 | 46076 | NSTTGGATNDNH | 7.26 | 8.16 | 0.93 | 4.25 |
| ATLVT012XX1145 | 46079 | NSTTGGSSNDNH | 7.06 | 7.38 | 0.79 | 3.51 |
| ATLVT012XX1148 | 46082 | NSTTGGSTNDNH | 8.27 | 8.69 | 0.68 | 3.72 |
| ATLVT012XX1571 | 46505 | SGQTGGASNDNH | 6.54 | 8.15 | 1.31 | 3.5 |
| ATLVT012XX1574 | 46508 | SGQTGGATNDNH | 9.6 | 12.38 | 0.01 | 4.01 |
| ATLVT012XX1577 | 46511 | SGQTGGSSNDNH | 3.37 | 4.17 | 1.49 | 2.66 |
| ATLVT012XX1580 | 46514 | SGQTGGSTNDNH | 8 | 8.23 | 0.82 | 4.32 |
| ATLVT012XX1620 | 46554 | SGTAGGASNDNT | 6.3 | 9.6 | 0.57 | 4.51 |
| ATLVT012XX1626 | 46560 | SGTAGGSSNDNT | 5.86 | 5.69 | 2.35 | 4.55 |
| ATLVT012XX1666 | 46600 | SGTSGGASNDNA | 4.83 | 5.82 | 2.01 | 3.56 |
| ATLVT012XX1669 | 46603 | SGTSGGATNDNA | 4.75 | 6.4 | 1.26 | 3.69 |
| ATLVT012XX1672 | 46606 | SGTSGGSSNDNA | 4.17 | 5.83 | 0.54 | 3.47 |
| ATLVT012XX1675 | 46609 | SGTSGGSTNDNA | 6.06 | 5.89 | 0.78 | 3.95 |
| ATLVT012XX1716 | 46650 | SGTTGGASNDNT | 4.4 | 5.75 | 1.26 | 2.61 |
| ATLVT012XX1719 | 46653 | SGTTGGATNDNT | 5.54 | 6.78 | 0.98 | 2.47 |
| ATLVT012XX1722 | 46656 | SGTTGGSSNDNT | 4.01 | 5.43 | 0.54 | 2.99 |
| ATLVT012XX1725 | 46659 | SGTTGGSTNDNT | 5.83 | 5.77 | 1.3 | 3.32 |
| ATLVT012XX2194 | 47128 | SSTAGGASNDNA | 6.46 | 7.2 | 0.11 | 3.67 |
| ATLVT012XX2197 | 47131 | SSTAGGATNDNA | 7.08 | 9.46 | 1.03 | 3.88 |
| ATLVT012XX2200 | 47134 | SSTAGGSSNDNA | 5.42 | 5.4 | 0.59 | 3.84 |
| ATLVT012XX2203 | 47137 | SSTAGGSTNDNA | 7.17 | 7.86 | −0.09 | 4.47 |
| ATLVT012XXAAV9 | 48388 | NSTSGGSSNDNA | 10.85 | 13.11 | 1.65 | 3.83 |
| ATLVT012XXMUT1 | 48390 | NSTSGASTNDNA | 3.84 | 2.84 | 0.6 | 2.27 |
| ATLVT012XXAAV9LIO | 48389 | NSTSGASSNDNA | 3.35 | 3.29 | 1.18 | 1.56 |

### 8.24. Example 24: Analysis of AAV^{-Lib1} in Non-Human Primate to Identify VRI Variants with Enhanced Muscle Tropism with Limited Liver Tropism

The objective of this study was to assess the 3,456 modified capsid proteins from the AAV^{-Lib1} in non-human primates (NHP) to identify capsids having VRI variants that enhance muscle tropism and limit liver tropism.

NHPs were administered the same AAV^{-Lib1} library as described in Example 15. Briefly, a library containing 3,456 unique sequences of modified AAV9 VP1 capsid proteins was produced, which is referred to herein as the AAV-Lil library or AAV^{-Lib1} capsid library. The peptide segment of 12 amino acids positioned between S261 and Y274 for each of the 3,456 modified AAV9 VP1 capsid sequence are as described in SEQ ID NOs: 251-3703. The amino acid modifications (insertions, deletions, substitutions) in the VRI region of AAV9 included those described herein as shown in FIG. 49A. Additionally, wild-type AAV9 and AAV^{mut1} capsid proteins are used as controls. Wild-type AAV9 is known to target the liver whereas AAV^{mut1} has a liver detargeting phenotype.

### Experimental Design

A total of 6 animals were divided into 2 groups as summarized in the below Table 18. ATV-0071-001 and ATV-0071-002 represent different lots of the AAV^{-Lib1} library. Immunosuppression of the animals began 7 days prior to vector administration. Group 1 and 2 were administered with the indicated amounts or concentrations of AAV^{-Lib1} (comprising the 3,456 modified capsid proteins, an AAV9 capsid protein, AAV^{mut1} capsid protein) by ICM or IV. Animals were sacrificed on day 32 after AAV vector administration and their organ samples are collected for analysis.

TABLE 18
Experimental design for non-human primate study.

| | | | Dose | | | |
|---|---|---|---|---|---|---|
| Group | | Dose | Vol | Dose (vg/ | Dose | Study end point |
| No. | Treatment | Route | (ml) | animal or kg) | Concentration | (necropsy day 32) |
| 1 | ATV- | ICM | 2.0 | 3.4E13 vg/animal | 1.7E13 | 3 (2 M/1 F) |
| | 0071-001 | | | | | |
| 2 | ATV- | IV | 10.0 | 5E13 vg/kg | 1.05E14* | 3 (3M) |
| | 0071-002 | | | | 9.0E13** | |

*Dose concentration for two animals
**Dose concentration for one animal

### Tissue Analysis

Successful gene transfer by AAV^{-Lib1} Library (and controls) was detected by NGS sequencing of DNA and/or RNA harvested from mouse tissue following IV injection of the AAV^{-Lib1} library. In particular, the NHP were sacrificed 32 days after the injection. Individual tissues, notably the liver, heart, and diaphragm, were collected at the time of necropsy. For DNA, tissues were homogenized in a Qiagen Tissuelyser II (20 rps for 2 min) in lysis buffer from the Qiagen Dneasy Blood and Tissue Kit or the Qiagen RNeasy Lipid Tissue Mini Kit following the standard Qiagen protocol. Samples were eluted in 50 μL of buffer. For RNA, tissues were immediately placed into the preservative RNAlater, after which the RNAlater was removed and the tissue flash frozen. Variations to these general protocols are as described below.

### AAV^{-Lib1} Library Screen Data Informatics Analysis

Next generation sequencing (NGS) was used to determine the presence of AAV vector genomes (i.e., DNA) and to determine expression of AAV components (i.e., viral RNA) such as capsid sequences encoded by the AAV vector DNA. Following DNA and/or RNA isolation, sequencing library preparation, and sequencing, the resulting FASTQ reads were subjected to SeqCount QC (i.e., quality control of sequencing reads) before determining Variant Counts. Variant Counts represent the total Variant Count (i.e., sequencing reads associated with a particular capsid variant) in a sample or input test article. Prior to determining tissue enrichment, Variant Count data was normalized. Normalization included two steps. Step 1 included sequencing depth normalization:

$$log 2 CPM = log 2 ( \frac{RawVariantCount + 0.5}{TotalVariantCount + 1} \times 1 , 000 , 000 )$$

Step 2 included distribution normalization by median:

$$log 2 MN = log 2 CPM - median ( log 2 CPM )$$

Tissue enrichment score was calculated using:

$$log 2 FC = log 2 {MN}_{Tissue} - log 2 {MN}_{TestArticle}$$

$$scaled log 2 FC = \frac{( log 2 FC - min ( log 2 FC ) )}{( max ( log 2 FC ) - min ( log 2 FC ) )}$$

Tissue enrichment scores for all 3,456 variants and controls are as shown in Table 30, which is herein incorporated by reference in its entirety. Table 30 reports tissue enrichment scores for Liver DNA, Heart DNA, and Diaphragm DNA as Log 2 Fold Change (FC) to test article. The tissue enrichment measurements were used for functional interpretation of the data as well as for variant ranking and selecting capsids from the AAV^{-Lib1} library.

DNA samples were analyzed for biodistribution of vector genomes in the liver, diaphragm, and hear using NGS. Data (i.e., AAV^{-Lib1} library data) for all 3.456 capsids and controls (AAV^{mut1} and AAV9) is found in Table 30, which is herein incorporated by reference in its entirety. Table 30 shows tissue enrichment scores Log 2 Fold Change (Fc) compared to test article. The top 200 modified AAV capsids from Table 30 are shown in Table 20.

TABLE 19
Top 200 Modified AAV Capsids from Appendix E. (AAV-Lib1 in NHP)

| | | SEQ | | | | |
|---|---|---|---|---|---|---|
| Rank | variant_id | ID NO: | Sequence | Diaphragm | Heart | Liver |
| 1 | ATLVT012XXAAV9 | 48388 | NSTSGGSSNDNA | 11.26 | 21.08 | 19.61 |
| 2 | ATLVT012XX1099 | 46033 | NSTSGGSTNDNA | 8.69 | 11.7 | 18.88 |
| 3 | ATLVT012XX1094 | 46028 | NSTSGGATNDNA | 9.77 | 11.36 | 16.69 |
| 4 | ATLVT012XX1100 | 46034 | NSTSGGSTNDNH | 8.96 | 12.75 | 16.17 |
| 5 | ATLVT012XX1097 | 46031 | NSTSGGSSNDNH | 8.93 | 11.1 | 17.04 |
| 6 | ATLVT012XX1098 | 46032 | NSTSGGSSNDNT | 7.1 | 12.65 | 17.96 |
| 7 | ATLVT012XX1054 | 45988 | NSTAGGSTNDNA | 9.44 | 8.55 | 18.55 |
| 8 | ATLVT012XX1718 | 46652 | SGTTGGATNDNH | 9.02 | 11.49 | 14.24 |
| 9 | ATLVT012XX1147 | 46081 | NSTTGGSTNDNA | 7.39 | 10.36 | 18.33 |
| 10 | ATLVT012XX1101 | 46035 | NSTSGGSTNDNT | 7.29 | 10.97 | 17.33 |
| 11 | ATLVT012XX1144 | 46078 | NSTTGGSSNDNA | 6.52 | 12.04 | 17.21 |
| 12 | ATLVT012XX1091 | 46025 | NSTSGGASNDNA | 6.47 | 11.28 | 17.71 |
| 13 | ATLVT012XX1051 | 45985 | NSTAGGSSNDNA | 6.16 | 11.23 | 18.3 |
| 14 | ATLVT012XX2246 | 47180 | SSTSGGATNDNH | 7.85 | 10.16 | 16.7 |
| 15 | ATLVT012XX1093 | 46027 | NSTSGGASNDNT | 6.54 | 9.78 | 18.77 |
| 16 | ATLVT012XX1149 | 46083 | NSTTGGSTNDNT | 6.24 | 10.29 | 18.46 |
| 17 | ATLVT012XX523 | 45457 | NGTSGGSSNDNA | 7.82 | 10.99 | 14.84 |
| 18 | ATLVT012XX1053 | 45987 | NSTAGGSSNDNT | 6.11 | 10.03 | 18.82 |
| 19 | ATLVT012XX2243 | 47177 | SSTSGGASNDNH | 7.76 | 10.44 | 15.33 |
| 20 | ATLVT012XX1096 | 46030 | NSTSGGATNDNT | 5.83 | 10.7 | 18.3 |
| 21 | ATLVT012XX1670 | 46604 | SGTSGGATNDNH | 7.4 | 10.6 | 14.42 |
| 22 | ATLVT012XX1484 | 46418 | SGQAGGSTNDNH | 6.56 | 11.83 | 13.35 |
| 23 | ATLVT012XX2245 | 47179 | SSTSGGATNDNA | 7.25 | 11.27 | 12.73 |
| 24 | ATLVT012XX1142 | 46076 | NSTTGGATNDNH | 8.32 | 9.91 | 12.26 |
| 25 | ATLVT012XX1667 | 46601 | SGTSGGASNDNH | 8.08 | 9.25 | 13.33 |
| 26 | ATLVT012XX1056 | 45990 | NSTAGGSTNDNT | 6.42 | 9 | 16.44 |
| 27 | ATLVT012XX1095 | 46029 | NSTSGGATNDNH | 7.35 | 9.55 | 13.74 |
| 28 | ATLVT012XX1146 | 46080 | NSTTGGSSNDNT | 4.38 | 9.72 | 18.52 |
| 29 | ATLVT012XX2294 | 47228 | SSTTGGATNDNH | 8.07 | 8.33 | 13.8 |
| 30 | ATLVT012XX1055 | 45989 | NSTAGGSTNDNH | 7.57 | 9.8 | 12.76 |
| 31 | ATLVT012XX1047 | 45981 | NSTAGGASNDNT | 7.32 | 6.43 | 17.28 |
| 32 | ATLVT012XX1045 | 45979 | NSTAGGASNDNA | 5.47 | 10.24 | 14.81 |
| 33 | ATLVT012XX1141 | 46075 | NSTTGGATNDNA | 8.09 | 9.81 | 10.79 |
| 34 | ATLVT012XX1092 | 46026 | NSTSGGASNDNH | 7.04 | 7.89 | 14.34 |
| 35 | ATLVT012XX528 | 45462 | NGTSGGSTNDNT | 6.23 | 7.79 | 15.85 |
| 36 | ATLVT012XX1675 | 46609 | SGTSGGSTNDNA | 8.34 | 7.56 | 12.42 |
| 37 | ATLVT012XX1148 | 46082 | NSTTGGSTNDNH | 7.59 | 7.87 | 13.09 |
| 38 | ATLVT012XX1676 | 46610 | SGTSGGSTNDNH | 7.35 | 8.76 | 12.31 |
| 39 | ATLVT012XX571 | 45505 | NGTTGGSSNDNA | 7.68 | 8.14 | 12.16 |
| 40 | ATLVT012XX1052 | 45986 | NSTAGGSSNDNH | 6.03 | 8.92 | 13.8 |
| 41 | ATLVT012XX1140 | 46074 | NSTTGGASNDNT | 4.62 | 9.31 | 15.6 |
| 42 | ATLVT012XX1529 | 46463 | SGQSGGSSNDNH | 10.13 | 6.32 | 9.6 |
| 43 | ATLVT012XX1666 | 46600 | SGTSGGASNDNA | 8.04 | 8.48 | 10.31 |
| 44 | ATLVT012XX1143 | 46077 | NSTTGGATNDNT | 6.1 | 7.87 | 14.37 |
| 45 | ATLVT012XX1580 | 46514 | SGQTGGSTNDNH | 7.4 | 9.22 | 10.25 |
| 46 | ATLVT012XX1574 | 46508 | SGQTGGATNDNH | 6.97 | 7.32 | 13.42 |
| 47 | ATLVT012XX2054 | 46988 | SSQAGGATNDNH | 5.35 | 9.86 | 12.93 |
| 48 | ATLVT012XX2251 | 47185 | SSTSGGSTNDNA | 5.82 | 10.22 | 11.43 |
| 49 | ATLVT012XX2248 | 47182 | SSTSGGSSNDNA | 7.39 | 9.43 | 9.71 |
| 50 | ATLVT012XX1532 | 46466 | SGQSGGSTNDNH | 5.25 | 9.97 | 12.2 |
| 51 | ATLVT012XX2242 | 47176 | SSTSGGASNDNA | 5.92 | 8.57 | 12.68 |
| 52 | ATLVT012XX526 | 45460 | NGTSGGSTNDNA | 6.74 | 7.48 | 12.57 |
| 53 | ATLVT012XX1244 | 46178 | SGASGGSTNDNH | 7.97 | 9.41 | 7.95 |
| 54 | ATLVT012XX1579 | 46513 | SGQTGGSTNDNA | 5.34 | 10.71 | 10.65 |
| 55 | ATLVT012XX1720 | 46654 | SGTTGGSSNDNA | 6.16 | 9.07 | 10.95 |
| 56 | ATLVT012XX2102 | 47036 | SSQSGGATNDNH | 5.66 | 9.05 | 11.81 |
| 57 | ATLVT012XX525 | 45459 | NGTSGGSSNDNT | 5.23 | 6.43 | 15.89 |
| 58 | ATLVT012XX2108 | 47042 | SSQSGGSTNDNH | 8.25 | 9.5 | 6.64 |
| 59 | ATLVT012XX1719 | 46653 | SGTTGGATNDNT | 6.96 | 8.91 | 9.59 |
| 60 | ATLVT012XX2252 | 47186 | SSTSGGSTNDNH | 7.08 | 9.42 | 8.72 |
| 61 | ATLVT012XX1715 | 46649 | SGTTGGASNDNH | 5.11 | 9.59 | 11.78 |
| 62 | ATLVT012XX1145 | 46079 | NSTTGGSSNDNH | 4.26 | 9.95 | 12.54 |
| 63 | ATLVT012XX1526 | 46460 | SGQSGGATNDNH | 5.71 | 7.86 | 12.68 |
| 64 | ATLVT012XX1668 | 46602 | SGTSGGASNDNT | 7.37 | 7.46 | 10.34 |
| 65 | ATLVT012XX527 | 45461 | NGTSGGSTNDNH | 6.47 | 8.51 | 10.52 |
| 66 | ATLVT012XX2244 | 47178 | SSTSGGASNDNT | 5.83 | 7.69 | 12.28 |
| 67 | ATLVT012XX1046 | 45980 | NSTAGGASNDNH | 6.06 | 6.08 | 13.61 |
| 68 | ATLVT012XX1049 | 45983 | NSTAGGATNDNH | 6.07 | 6.27 | 13.11 |
| 69 | ATLVT012XX1531 | 46465 | SGQSGGSTNDNA | 5.24 | 9.4 | 10.5 |
| 70 | ATLVT012XX3446 | 48380 | TSTTGGATNDNH | 6.59 | 9.29 | 8.31 |
| 71 | ATLVT012XX1671 | 46605 | SGTSGGATNDNT | 4.9 | 9.38 | 10.77 |
| 72 | ATLVT012XX2250 | 47184 | SSTSGGSSNDNT | 7.72 | 6.67 | 9.37 |
| 73 | ATLVT012XX2203 | 47137 | SSTAGGSTNDNA | 5.49 | 9.59 | 9.37 |
| 74 | ATLVT012XX1814 | 46748 | SSASGGATNDNH | 6.43 | 10.77 | 6.23 |
| 75 | ATLVT012XX2291 | 47225 | SSTTGGASNDNH | 5.52 | 11.37 | 6.81 |
| 76 | ATLVT012XX1138 | 46072 | NSTTGGASNDNA | 4.8 | 6.48 | 14.2 |
| 77 | ATLVT012XX1139 | 46073 | NSTTGGASNDNH | 5.25 | 8.78 | 10.45 |
| 78 | ATLVT012XX1050 | 45984 | NSTAGGATNDNT | 5.09 | 6.01 | 14.28 |
| 79 | ATLVT012XX576 | 45510 | NGTTGGSTNDNT | 6.45 | 6.36 | 11.49 |
| 80 | ATLVT012XX2249 | 47183 | SSTSGGSSNDNH | 7.34 | 7.88 | 7.98 |
| 81 | ATLVT012XX524 | 45458 | NGTSGGSSNDNH | 6.4 | 6.89 | 10.8 |
| 82 | ATLVT012XX1533 | 46467 | SGQSGGSTNDNT | 3.5 | 11.85 | 9.36 |
| 83 | ATLVT012XX1528 | 46462 | SGQSGGSSNDNA | 7.93 | 6.53 | 8.46 |
| 84 | ATLVT012XX1382 | 46316 | SGESGGATNDNH | 8.11 | 6.14 | 8.62 |
| 85 | ATLVT012XX3395 | 48329 | TSTSGGASNDNH | 4.27 | 10.33 | 9.68 |
| 86 | ATLVT012XX1523 | 46457 | SGQSGGASNDNH | 5.7 | 7.33 | 10.93 |
| 87 | ATLVT012XX960 | 45894 | NSQSGGSTNDNT | 8.43 | 5.47 | 8.52 |
| 88 | ATLVT012XX1238 | 46172 | SGASGGATNDNH | 5.37 | 7.89 | 10.65 |
| 89 | ATLVT012XX2300 | 47234 | SSTTGGSTNDNH | 7.18 | 8.2 | 7.07 |
| 90 | ATLVT012XX1714 | 46648 | SGTTGGASNDNA | 6.11 | 8.36 | 8.66 |
| 91 | ATLVT012XX521 | 45455 | NGTSGGATNDNH | 7.45 | 5.79 | 9.66 |
| 92 | ATLVT012XX1717 | 46651 | SGTTGGATNDNA | 5.45 | 9.22 | 8.67 |
| 93 | ATLVT012XX1475 | 46409 | SGQAGGASNDNH | 4.94 | 7.89 | 11.24 |
| 94 | ATLVT012XX574 | 45508 | NGTTGGSTNDNA | 6.54 | 7.27 | 9.29 |
| 95 | ATLVT012XX1723 | 46657 | SGTTGGSTNDNA | 6.19 | 7.07 | 9.96 |
| 96 | ATLVT012XX1724 | 46658 | SGTTGGSTNDNH | 5.42 | 8.09 | 9.92 |
| 97 | ATLVT012XX1722 | 46656 | SGTTGGSSNDNT | 5.89 | 7.3 | 10.09 |
| 98 | ATLVT012XX1672 | 46606 | SGTSGGSSNDNA | 5.82 | 6.96 | 10.63 |
| 99 | ATLVT012XXMUT1 | 48390 | NSTSGASTNDNA | 3.73 | 10.26 | 9.82 |
| 100 | ATLVT012XX517 | 45451 | NGTSGGASNDNA | 5.54 | 8.57 | 8.74 |
| 101 | ATLVT012XX575 | 45509 | NGTTGGSTNDNH | 6.44 | 6.48 | 9.76 |
| 102 | ATLVT012XX3400 | 48334 | TSTSGGSSNDNA | 3.09 | 11.22 | 9.38 |
| 103 | ATLVT012XX2150 | 47084 | SSQTGGATNDNH | 4.32 | 6.83 | 12.91 |
| 104 | ATLVT012XX519 | 45453 | NGTSGGASNDNT | 6.54 | 5.74 | 10.47 |
| 105 | ATLVT012XX1485 | 46419 | SGQAGGSTNDNT | 6.45 | 6.19 | 10.03 |
| 106 | ATLVT012XX1483 | 46417 | SGQAGGSTNDNA | 6.14 | 6.75 | 9.82 |
| 107 | ATLVT012XX518 | 45452 | NGTSGGASNDNH | 8.18 | 4.75 | 8.86 |
| 108 | ATLVT012XX1622 | 46556 | SGTAGGATNDNH | 3.96 | 8 | 11.85 |
| 109 | ATLVT012XX1478 | 46412 | SGQAGGATNDNH | 3.81 | 9.14 | 10.57 |
| 110 | ATLVT012XX3398 | 48332 | TSTSGGATNDNH | 4.01 | 9.02 | 10.32 |
| 111 | ATLVT012XX1669 | 46603 | SGTSGGATNDNA | 6.42 | 6.39 | 9.37 |
| 112 | ATLVT012XX520 | 45454 | NGTSGGATNDNA | 7.24 | 6.05 | 8.2 |
| 113 | ATLVT012XX2253 | 47187 | SSTSGGSTNDNT | 5.41 | 7.59 | 9.25 |
| 114 | ATLVT012XX2299 | 47233 | SSTTGGSTNDNA | 7.26 | 5.95 | 8.2 |
| 115 | ATLVT012XX2870 | 47804 | TGTTGGATNDNH | 5.09 | 6.66 | 10.78 |
| 116 | ATLVT012XX2099 | 47033 | SSQSGGASNDNH | 6.03 | 6.58 | 9.21 |
| 117 | ATLVT012XX1628 | 46562 | SGTAGGSTNDNH | 3.87 | 9.58 | 8.95 |
| 118 | ATLVT012XX522 | 45456 | NGTSGGATNDNT | 4.75 | 6.91 | 10.8 |
| 119 | ATLVT012XX3403 | 48337 | TSTSGGSTNDNA | 4.34 | 8.42 | 9.53 |
| 120 | ATLVT012XX1048 | 45982 | NSTAGGATNDNA | 4.45 | 6.29 | 11.92 |
| 121 | ATLVT012XX1530 | 46464 | SGQSGGSSNDNT | 6.93 | 6.32 | 7.59 |
| 122 | ATLVT012XX477 | 45411 | NGTAGGSSNDNT | 3.86 | 7.4 | 11.31 |
| 123 | ATLVT012XX1436 | 46370 | SGETGGSTNDNH | 7.84 | 6.48 | 5.47 |
| 124 | ATLVT012XX573 | 45507 | NGTTGGSSNDNT | 4.16 | 7.38 | 10.63 |
| 125 | ATLVT012XX1430 | 46364 | SGETGGATNDNH | 4.68 | 7.69 | 9.3 |
| 126 | ATLVT012XX2247 | 47181 | SSTSGGATNDNT | 3.72 | 6.99 | 11.63 |
| 127 | ATLVT012XX1627 | 46561 | SGTAGGSTNDNA | 4.84 | 5.43 | 11.7 |
| 128 | ATLVT012XX1725 | 46659 | SGTTGGSTNDNT | 3.93 | 7.56 | 10.49 |
| 129 | ATLVT012XX2293 | 47227 | SSTTGGATNDNA | 4.5 | 9.42 | 6.94 |
| 130 | ATLVT012XX3302 | 48236 | TSQTGGATNDNH | 3.59 | 6.48 | 12.1 |
| 131 | ATLVT012XX1721 | 46655 | SGTTGGSSNDNH | 6.18 | 5.93 | 8.38 |
| 132 | ATLVT012XX3404 | 48338 | TSTSGGSTNDNH | 5.74 | 7.42 | 7.14 |
| 133 | ATLVT012XX3397 | 48331 | TSTSGGATNDNA | 5.77 | 6.41 | 8.22 |
| 134 | ATLVT012XX1623 | 46557 | SGTAGGATNDNT | 2.9 | 9.91 | 8.42 |
| 135 | ATLVT012XX2774 | 47708 | TGTAGGATNDNH | 7.08 | 2.26 | 11.07 |
| 136 | ATLVT012XX1626 | 46560 | SGTAGGSSNDNT | 2.66 | 9.25 | 9.64 |
| 137 | ATLVT012XX1958 | 46892 | SSESGGATNDNH | 7.1 | 7.32 | 4.5 |
| 138 | ATLVT012XX1527 | 46461 | SGQSGGATNDNT | 3.51 | 8.49 | 9.03 |
| 139 | ATLVT012XX2198 | 47132 | SSTAGGATNDNH | 4.15 | 3.92 | 13.77 |
| 140 | ATLVT012XX1677 | 46611 | SGTSGGSTNDNT | 4.49 | 5.49 | 11.15 |
| 141 | ATLVT012XX2290 | 47224 | SSTTGGASNDNA | 5.14 | 8.62 | 5.88 |
| 142 | ATLVT012XX2197 | 47131 | SSTAGGATNDNA | 3.98 | 6.27 | 10.88 |
| 143 | ATLVT012XX3299 | 48233 | TSQTGGASNDNH | 4.31 | 8.29 | 7.7 |
| 144 | ATLVT012XX3394 | 48328 | TSTSGGASNDNA | 2.55 | 9.48 | 9.14 |
| 145 | ATLVT012XX1916 | 46850 | SSEAGGSTNDNH | 8.85 | 5.76 | 3.08 |
| 146 | ATLVT012XX1243 | 46177 | SGASGGSTNDNA | 6.82 | 6.59 | 5.49 |
| 147 | ATLVT012XX1674 | 46608 | SGTSGGSSNDNT | 5.84 | 4.7 | 9.59 |
| 148 | ATLVT012XX2061 | 46995 | SSQAGGSTNDNT | 4.8 | 6.96 | 8.42 |
| 149 | ATLVT012XX1474 | 46408 | SGQAGGASNDNA | 3.52 | 8.55 | 8.39 |
| 150 | ATLVT012XX959 | 45893 | NSQSGGSTNDNH | 8.66 | 5.18 | 3.92 |
| 151 | ATLVT012XX1716 | 46650 | SGTTGGASNDNT | 4.75 | 4.39 | 11.62 |
| 152 | ATLVT012XXAAV9LIO | 48389 | NSTSGASSNDNA | 4.36 | 8.76 | 6.67 |
| 153 | ATLVT012XX1673 | 46607 | SGTSGGSSNDNH | 5.57 | 5.83 | 8.32 |
| 154 | ATLVT012XX2107 | 47041 | SSQSGGSTNDNA | 6.01 | 6.96 | 6.09 |
| 155 | ATLVT012XX1571 | 46505 | SGQTGGASNDNH | 3.83 | 6.45 | 10.38 |
| 156 | ATLVT012XX2819 | 47753 | TGTSGGASNDNH | 7.29 | 4.28 | 7.16 |
| 157 | ATLVT012XX2205 | 47139 | SSTAGGSTNDNT | 4.93 | 5.74 | 9.34 |
| 158 | ATLVT012XX471 | 45405 | NGTAGGASNDNT | 6.34 | 4.98 | 7.6 |
| 159 | ATLVT012XX2301 | 47235 | SSTTGGSTNDNT | 5.68 | 5.01 | 8.67 |
| 160 | ATLVT012XX1581 | 46515 | SGQTGGSTNDNT | 3.22 | 7.96 | 9.04 |
| 161 | ATLVT012XX2201 | 47135 | SSTAGGSSNDNH | 4.61 | 7.83 | 6.73 |
| 162 | ATLVT012XX2295 | 47229 | SSTTGGATNDNT | 3.68 | 7.97 | 8.01 |
| 163 | ATLVT012XX1577 | 46511 | SGQTGGSSNDNH | 5.49 | 6.96 | 6.22 |
| 164 | ATLVT012XX1624 | 46558 | SGTAGGSSNDNA | 2.93 | 6.5 | 11.21 |
| 165 | ATLVT012XX1196 | 46130 | SGAAGGSTNDNH | 2.79 | 7.46 | 10.12 |
| 166 | ATLVT012XX1573 | 46507 | SGQTGGATNDNA | 7.22 | 3.74 | 7.2 |
| 167 | ATLVT012XX2296 | 47230 | SSTTGGSSNDNA | 6.39 | 5.03 | 6.97 |
| 168 | ATLVT012XX569 | 45503 | NGTTGGATNDNH | 5.06 | 5.79 | 8.16 |
| 169 | ATLVT012XX1235 | 46169 | SGASGGASNDNH | 6.83 | 6.1 | 4.67 |
| 170 | ATLVT012XX958 | 45892 | NSQSGGSTNDNA | 7.97 | 5.14 | 3.95 |
| 171 | ATLVT012XX2109 | 47043 | SSQSGGSTNDNT | 6.08 | 6.47 | 5.47 |
| 172 | ATLVT012XX567 | 45501 | NGTTGGASNDNT | 3.12 | 7.07 | 9.59 |
| 173 | ATLVT012XX1570 | 46504 | SGQTGGASNDNA | 3.57 | 7.03 | 8.77 |
| 174 | ATLVT012XX2105 | 47039 | SSQSGGSSNDNH | 6.35 | 6.17 | 5.03 |
| 175 | ATLVT012XX2195 | 47129 | SSTAGGASNDNH | 4.6 | 4 | 10.75 |
| 176 | ATLVT012XX480 | 45414 | NGTAGGSTNDNT | 4.07 | 4.75 | 10.69 |
| 177 | ATLVT012XX3448 | 48382 | TSTTGGSSNDNA | 6.03 | 5.42 | 6.42 |
| 178 | ATLVT012XX1598 | 46532 | SGTAAGATNDNH | 5.76 | 8.56 | 2.75 |
| 179 | ATLVT012XX3444 | 48378 | TSTTGGASNDNT | 6.54 | 4.36 | 6.7 |
| 180 | ATLVT012XX572 | 45506 | NGTTGGSSNDNH | 4.56 | 5.62 | 8.33 |
| 181 | ATLVT012XX3401 | 48335 | TSTSGGSSNDNH | 5.01 | 5.79 | 7.29 |
| 182 | ATLVT012XX2200 | 47134 | SSTAGGSSNDNA | 3.92 | 5.57 | 9.37 |
| 183 | ATLVT012XX1090 | 46024 | NSTSGASTNDNT | 4.67 | 5.99 | 7.49 |
| 184 | ATLVT012XX2147 | 47081 | SSQTGGASNDNH | 4.03 | 6.06 | 8.45 |
| 185 | ATLVT012XX1077 | 46011 | NSTSAGSSNDNT | 1.63 | 9.84 | 7.69 |
| 186 | ATLVT012XX1629 | 46563 | SGTAGGSTNDNT | 3.09 | 7.28 | 8.43 |
| 187 | ATLVT012XX3451 | 48385 | TSTTGGSTNDNA | 4.37 | 6.17 | 7.66 |
| 188 | ATLVT012XX1388 | 46322 | SGESGGSTNDNH | 4.65 | 5.66 | 7.75 |
| 189 | ATLVT012XX2156 | 47090 | SSQTGGSTNDNH | 5.57 | 6.34 | 5.22 |
| 190 | ATLVT012XX1819 | 46753 | SSASGGSTNDNA | 5.4 | 7.14 | 4.49 |
| 191 | ATLVT012XX1524 | 46458 | SGQSGGASNDNT | 4.45 | 4.74 | 9.19 |
| 192 | ATLVT012XX1130 | 46064 | NSTTGAATNDNH | 5.66 | 4.61 | 7.25 |
| 193 | ATLVT012XX3308 | 48242 | TSQTGGSTNDNH | 3.3 | 9.43 | 5.04 |
| 194 | ATLVT012XX2822 | 47756 | TGTSGGATNDNH | 4.48 | 6.38 | 6.95 |
| 195 | ATLVT012XX3396 | 48330 | TSTSGGASNDNT | 3.02 | 6.68 | 9.03 |
| 196 | ATLVT012XX570 | 45504 | NGTTGGATNDNT | 3.15 | 8 | 7.12 |
| 197 | ATLVT012XX478 | 45412 | NGTAGGSTNDNA | 3.12 | 5.21 | 10.46 |
| 198 | ATLVT012XX1480 | 46414 | SGQAGGSSNDNA | 4.38 | 5.92 | 7.29 |
| 199 | ATLVT012XX1084 | 46018 | NSTSGAATNDNA | 5.48 | 5.24 | 6.28 |
| 200 | ATLVT012XX2060 | 46994 | SSQAGGSTNDNH | 5 | 4.44 | 8.01 |

As the overall aim of the AAV^{-Lib1} library study was to identify AAV capsid proteins that decrease liver tropism and increase muscle tropism for at least have comparable muscle tropism to a control (e.g., AAV9)), additional analysis was performed to determine which peptide segments could produce the desired tropism.

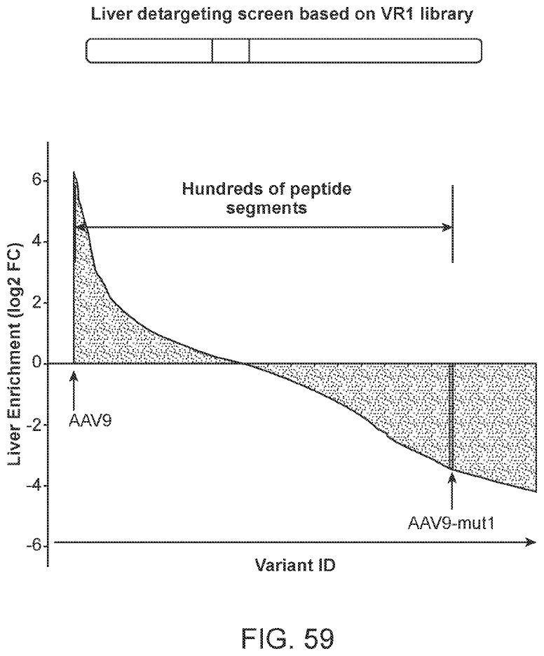

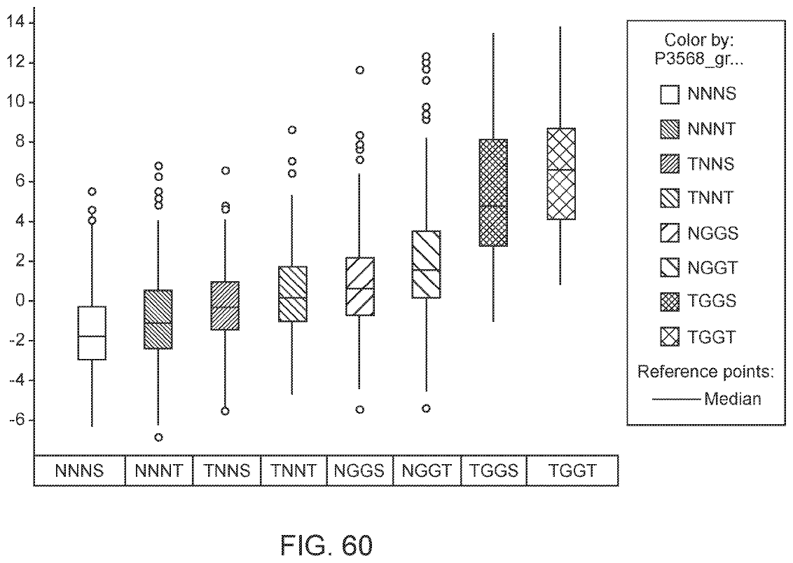

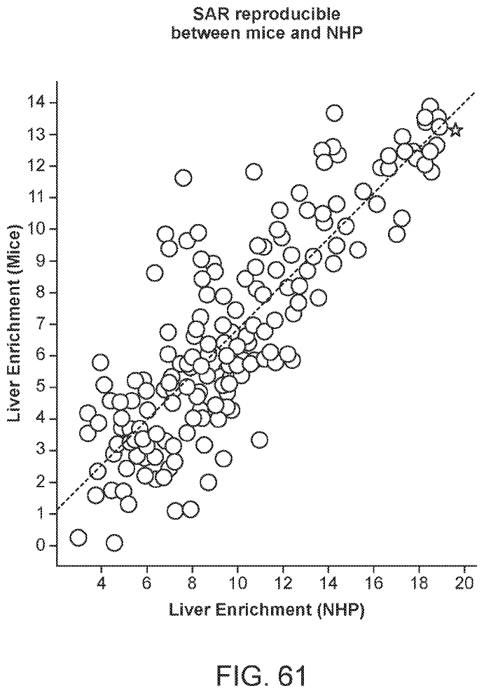

The data in Table 30 was used to identify interesting peptide segments, for example, peptide segments with liver enrichment between AAV9 and AAV9-mut 1 (see FIG. 59). Using the sequence data, enrichment scores for each tissue were calculated for each AAV variant containing a unique targeting peptide. In particular, the AAV variants including each targeting peptide were ranked based on a mean log fold-change tissue score (see, e.g., Table 30). After determining a sequence count from sequence read data (FASTQ) following sequencing. AAV variants containing a unique targeting peptide were counted and normalized, followed by performing a tissue enrichment analysis for each tissue. Tissue enrichment analysis included Sequence Activity Relationship (SAR) analysis, Network analysis, and structural modeling. SAR analysis identified particular amino acid at a specific capsid position or sequence motifs (combination of amino acids at several positions) that significantly affects tissue tropism. Network analysis identified modules of variants that shared amino acid or peptide sequences of top performing variants. Structural modeling provided understanding of the structural effect of significant amino acids at specific capsid positions for mechanistic hypothesis formulation. SAR analysis was performed on the peptide segments having liver enrichment between AAV9 and AAV9-mut1. As shown in FIG. 60, this identified sequence motifs at four different positions. SAR analysis was reproducible (i.e., showed good correlation) between NHP and mice (see FIG. 61).

Taken together, this Example showed that sequence activity relationship (SAR) analysis of the AAV^{-Lib1} library data could be used to determine which amino acid modifications at which residues or which peptide segments in VR I impact liver tropism.

### 8.25. Example 25: Assessment of AAVs Having Capsids a Targeting Peptide in VR VIII and a Peptide Segment within VR I in Mice and Non-Human Primates

The objective of this study was to assess AAVs comprising a capsid protein having a targeting peptide in VR VIII and a tropism-altering peptide segment within VR I in non-human primates (NHP) to identify heart neutral, liver detargeted. AAVs (e.g., moderate to low liver expression compared to a control (e.g., AAV9)) with potential for muscle targeting. Overall 28 peptide segments and 12 targeting peptides were selected for assessment.

Briefly, as noted above, the modified AAV capsid protein included peptide segments (e.g., the peptide segments described in Table 20) positioned between S261 and Y274 in VR I of AAV9. 28 peptide segments were selected based at least in part, on the data in Table 30, which is herein incorporated by reference in its entirety, including amino acid sequences selected from SEQ ID NO: 46026, 46029, 46031, 46035, 46073, 46076, 46079, 46082, 46505, 46508, 46511, 46514, 46554, 46560, 46609, 46600, 46603, 46606, 46650, 46653, 46656, 46659, 47128, 47131, 47134, and 47137. The 28 peptide segments can be divided into seven groups that are represented by SEQ ID NOs: 44927-44933, where each SEQ ID NO represents one of the seven groups. Six of the seven group include a threonine (T), a glycine (G), and a glycine (G) at positions P₃, P₅, and P₆, respectively, within the peptide segment. One group includes a glutamine (Q), a glycine (G), and a glycine (G) at positions P₃, P₅, and P₆, respectively, within the peptide segment. Within each of the seven groups, amino acid position P₇ is varied between an alanine (A) and a serine (S) and amino acid position P₈ is varied between a serine (S) and a threonine (T). Four of the seven groups (i.e., SEQ ID NOs: 44927-44933) were selected for testing in a modified AAV capsid protein that also includes a targeting peptide.

Briefly, the eight targeting peptides selected for testing in this example included targeting peptides having an amino acid sequence selected from SEQ ID NO: 44864-44867, 44879-44883, 44911, 44912, 44913, 44918, 44919.

Overall, in this experiment 116 AAV capsid proteins were assessed. AAV9 capsids were modified to include (i) the targeting peptide at a site within VR VIII of the reference AAV capsid protein and (ii) one or more modifications to comprise a peptide segment within variable region I (VR I) of the reference AAV capsid protein as described in Table 20

TABLE 20
Combinations of Targeting Peptides and Peptide Segments

| | | SEQ ID | | SEQ ID |
|---|---|---|---|---|
| Combinations | VR1 | NO: | VR8 | NO: |
| 1 | NSTSGGASNDNH | 46026 | wt VR8 | 48388 |
| 2 | NSTSGGATNDNH | 46029 | wt VR8 | 48388 |
| 3 | NSTSGGSSNDNH | 46031 | wt VR8 | 48388 |
| 4 | NSTSGGSTNDNH | 46034 | wt VR8 | 48388 |
| 5 | NSTTGGASNDNH | 46073 | wt VR8 | 48388 |
| 6 | NSTTGGATNDNH | 46076 | wt VR8 | 48388 |
| 7 | NSTTGGSSNDNH | 46079 | wt VR8 | 48388 |
| 8 | NSTTGGSTNDNH | 46082 | wt VR8 | 48388 |
| 9 | SGQTGGASNDNH | 46505 | wt VR8 | 48388 |
| 10 | SGQTGGATNDNH | 46508 | wt VR8 | 48388 |
| 11 | SGQTGGSSNDNH | 46511 | wt VR8 | 48388 |
| 12 | SGQTGGSTNDNH | 46514 | wt VR8 | 48388 |
| 13 | SGTAGGASNDNT | 46554 | ENR RGD EQNT | 44867 |
| 14 | SGTAGGASNDNT | 46554 | ENR RGD FNGL | 44879 |
| 15 | SGTAGGASNDNT | 46554 | ENR RGD FNNL | 44864 |
| 16 | SGTAGGASNDNT | 46554 | ENR RGD FNNT | 44880 |
| 17 | SGTAGGASNDNT | 46554 | ENR RGD FNST | 44865 |
| 18 | SGTAGGASNDNT | 46554 | SAQ RGD FNNT | 44881 |
| 19 | SGTAGGASNDNT | 46554 | SAQ RGD LLLS | 44882 |
| 20 | SGTAGGASNDNT | 46554 | SNR RGD FNNT | 44883 |
| 21 | SGTAGGASNDNT | 46554 | ENR RGD FQNT | 44866 |
| 22 | SGTAGGASNDNT | 46554 | SAQ RGD RGQI | 44911 |
| 23 | SGTAGGASNDNT | 46554 | SAQ RGD RGVV | 44918 |
| 24 | SGTAGGASNDNT | 46554 | SAQ RGD HVNL | 44919 |
| 25 | SGTAGGASNDNT | 46554 | wt VR8 | 48388 |
| 26 | SGTAGGATNDNT | 46557 | wt VR8 | 48388 |
| 27 | SGTAGGSSNDNT | 46560 | wt VR8 | 48388 |
| 28 | SGTAGGSTNDNT | 46563 | wt VR8 | 48388 |
| 29 | SGTSGGSTNDNA | 46609 | ENR RGD EQNT | 44867 |
| 30 | SGTSGGSTNDNA | 46609 | ENR RGD FNGL | 44879 |
| 31 | SGTSGGSTNDNA | 46609 | ENR RGD FNNL | 44864 |
| 32 | SGTSGGSTNDNA | 46609 | ENR RGD FNNT | 44880 |
| 33 | SGTSGGSTNDNA | 46609 | ENR RGD FNST | 44865 |
| 34 | SGTSGGSTNDNA | 46609 | SAQ RGD FNNT | 44881 |
| 35 | SGTSGGSTNDNA | 46609 | SAQ RGD LLLS | 44882 |
| 36 | SGTSGGSTNDNA | 46609 | SNR RGD FNNT | 44883 |
| 37 | SGTSGGSTNDNA | 46609 | ENR RGD FQNT | 44866 |
| 38 | SGTSGGSTNDNA | 46609 | SAQ RGD RGQI | 44911 |
| 39 | SGTSGGSTNDNA | 46609 | SAQ RGD RGVV | 44918 |
| 40 | SGTSGGSTNDNA | 46609 | SAQ RGD HVNL | 44919 |
| 41 | SGTSGGASNDNA | 46600 | wt VR8 | 48388 |
| 42 | SGTSGGATNDNA | 46603 | wt VR8 | 48388 |
| 43 | SGTSGGSSNDNA | 46606 | wt VR8 | 48388 |
| 44 | SGTSGGSTNDNA | 46609 | wt VR8 | 48388 |
| 45 | SGTTGGSTNDNT | 46659 | ENR RGD EQNT | 44867 |
| 46 | SGTTGGSTNDNT | 46659 | ENR RGD FNGL | 44879 |
| 47 | SGTTGGSTNDNT | 46659 | ENR RGD FNNL | 44864 |
| 48 | SGTTGGSTNDNT | 46659 | ENR RGD FNNT | 44880 |
| 49 | SGTTGGSTNDNT | 46659 | ENR RGD FNST | 44865 |
| 50 | SGTTGGSTNDNT | 46659 | SAQ RGD FNNT | 44881 |
| 51 | SGTTGGSTNDNT | 46659 | SAQ RGD LLLS | 44882 |
| 52 | SGTTGGSTNDNT | 46659 | SNR RGD FNNT | 44883 |
| 53 | SGTTGGSTNDNT | 46659 | ENR RGD FQNT | 44866 |
| 54 | SGTTGGSTNDNT | 46659 | SAQ RGD RGQI | 44911 |
| 55 | SGTTGGSTNDNT | 46659 | SAQ RGD RGVV | 44918 |
| 56 | SGTTGGSTNDNT | 46659 | SAQ RGD HVNL | 44919 |
| 57 | SGTTGGASNDNT | 46650 | wt VR8 | 48388 |
| 58 | SGTTGGATNDNT | 46653 | wt VR8 | 48388 |
| 59 | SGTTGGSSNDNT | 46656 | wt VR8 | 48388 |
| 60 | SGTTGGSTNDNT | 46659 | wt VR8 | 48388 |
| 61 | SSTAGGASNDNA | 47128 | ENR RGD EQNT | 44867 |
| 62 | SSTAGGASNDNA | 47128 | ENR RGD FNGL | 44879 |
| 63 | SSTAGGASNDNA | 47128 | ENR RGD FNNL | 44864 |
| 64 | SSTAGGASNDNA | 47128 | ENR RGD FNNT | 44880 |
| 65 | SSTAGGASNDNA | 47128 | ENR RGD FNST | 44865 |
| 66 | SSTAGGASNDNA | 47128 | SAQ RGD FNNT | 44881 |
| 67 | SSTAGGASNDNA | 47128 | SAQ RGD LLLS | 44882 |
| 68 | SSTAGGASNDNA | 47128 | SNR RGD FNNT | 44883 |
| 69 | SSTAGGASNDNA | 47128 | ENR RGD FQNT | 44866 |
| 70 | SSTAGGASNDNA | 47128 | SAQ RGD RGQI | 44911 |
| 71 | SSTAGGASNDNA | 47128 | SAQ RGD RGVV | 44918 |
| 672 | SSTAGGASNDNA | 47128 | SAQ RGD HVNL | 44919 |
| 73 | SSTAGGASNDNA | 47128 | wt VR8 | 48388 |
| 74 | SSTAGGATNDNA | 47131 | wt VR8 | 48388 |
| 75 | SSTAGGSSNDNA | 47134 | wt VR8 | 48388 |
| 76 | SSTAGGSTNDNA | 47137 | wt VR8 | 48388 |
| 77 | w/t | 48388 | SNR RGD FNNT | 44867 |
| 78 | w/t | 48388 | ENR RGD FNNT | 44879 |
| 79 | w/t | 48388 | ENR RGD EQNT | 44864 |
| 80 | w/t | 48388 | SAQ RGD LLLS | 44880 |
| 81 | w/t | 48388 | ENR RGD FNST | 44865 |
| 82 | w/t | 48388 | ENR RGD FNNL | 44881 |
| 83 | w/t | 48388 | SAQ RGD FNNT | 44882 |
| 84 | w/t | 48388 | ENR RGD FNGL | 44883 |
| 85 | w/t | 48388 | ENR RGD FQNT | 44866 |
| 86 | w/t | 48388 | SAQ RGD RGQI | 44911 |
| 87 | w/t | 48388 | SAQ RGD RGVV | 44918 |
| 88 | w/t | 48388 | SAQ RGD HVNL | 44919 |
| 89 | w/t | 48388 | w/t | 48388 |
| 90 | NSTSGASTNDNA | 48390 | SNR RGD FNNT | 44867 |
| 91 | NSTSGASTNDNA | 48390 | ENR RGD FNNT | 44879 |
| 92 | NSTSGASTNDNA | 48390 | ENR RGD EQNT | 44864 |
| 93 | NSTSGASTNDNA | 48390 | SAQ RGD LLLS | 44880 |
| 94 | NSTSGASTNDNA | 48390 | ENR RGD FNST | 44865 |
| 95 | NSTSGASTNDNA | 48390 | ENR RGD FNNL | 44881 |
| 96 | NSTSGASTNDNA | 48390 | SAQ RGD FNNT | 44882 |
| 97 | NSTSGASTNDNA | 48390 | ENR RGD FNGL | 44883 |
| 98 | NSTSGASTNDNA | 48390 | ENR RGD FQNT | 44866 |
| 99 | NSTSGASTNDNA | 48390 | SAQ RGD RGQI | 44911 |
| 100 | NSTSGASTNDNA | 48390 | SAQ RGD RGVV | 44918 |
| 101 | NSTSGASTNDNA | 48390 | SAQ RGD HVNL | 44919 |
| 102 | NSTSGASTNDNA | 48390 | w/t | 48388 |
| 103 | SGTTGGSSNDNT | 46656 | ENRRGDFNNL | 44864 |
| 104 | SGTTGGSSNDNT | 46656 | SAQRGDLLLS | 44882 |
| 105 | SGTTGGSSNDNT | 46656 | ENRRGDFQNT | 44866 |
| 106 | SGTTGGSSNDNT | 46656 | SAQRGDRQGI | 44912 |
| 107 | SGTTGGSSNDNT | 46656 | SAQRGDRGVV | 44918 |
| 108 | SGTTGGSSNDNT | 46656 | SAQRGDHVNL | 44919 |
| 109 | SGTTGGSSNDNT | 46656 | ENRRGDFNNT | 44880 |
| 110 | SSTAGGATNDNA | 47131 | SAQRGDLLLS | 44882 |
| 111 | SSTAGGATNDNA | 47131 | ENRRGDFQNT | 44866 |
| 112 | SSTAGGATNDNA | 47131 | SAQRGDRQGI | 44912 |
| 113 | SSTAGGATNDNA | 47131 | SAQRGDRGVV | 44918 |
| 114 | SSTAGGATNDNA | 47131 | SAQRGDHVNL | 44919 |
| 115 | SSTAGGATNDNA | 47131 | ENRRGDFNNT | 44880 |
| 116 | SSTAGGATNDNA | 47131 | ENRRGDFNNL | 44864 |

A library containing the 116 AAV capsids comprising the targeting peptides and/or the peptide segments described in Table 21 was produced according to the methods described herein. This library is referred to herein as the AAV-mini library or AAV-mini capsid library. Additionally, wild-type AAV9 and AAV^{mut1} capsid proteins were used as controls. Wild-type AAV9 is known to target the liver whereas AAV^{mut1} has a liver detargeting phenotype. The AAV-mini library was assessed in mice and non-human primates (NHPs).

### 8.25.1. AAV-Mini Library in NHPs

### Experimental Design

A total of 3 animals were used for this study according to the experimental design in Table 21. Immunosuppression of the animals began 7 days prior to AAV vector administration. NHPs were administered with the indicated amounts or concentrations of AAV^{-mini} library (comprising the 116 modified capsid proteins of Table 20) by IV. Table 21. Experimental design for non-human primate study.

TABLE 21

| | | | | | Study end |
|---|---|---|---|---|---|
| Group | | Dose | Dose | Dose (vg/ | point (necropsy |
| No. | Treatment | Route | Vol (ml) | animal or kg) | day 28) |
| 1 | Mini- | IV | 10.0 | 5E13 vg/kg | 3 |
| | Library | | | | |

Animals were sacrificed on day 28 after AAV vector administration and heart, liver, diaphragm, and quadricep samples were collected for analysis. Tissues were immediately placed into the preservative RNAlater, after which the RNAlater was removed and the tissue flash frozen. RNA was isolated from the tissue samples and sequenced to identify the AAVs (AAVs from AAV^{-mini} library, each AAV containing a unique targeting peptide in VR VIII and a unique peptide segment in VR I) localized in each tissue. Using the sequence data, enrichment scores for each tissue were calculated for each AAV variant (calculated as described in Example 11). After determining a sequence count from sequence read data (FASTQ) following sequencing, AAV variants were counted and normalized. The AAV variants were assigned tissue enrichment scores for each individual tissue analyzed (i.e., heart, liver, diaphragm, and quadriceps, biceps, gastrocnemius, and Tibialis). Tissue enrichment data for the AAV^{-mini} library in NHP is shown in Table 23 with tissue enrichment scores in columns F-L for the respective tissues.

TABLE 22

| | | | | VR | | | | | | | | | | |
|---|---|---|---|---|---|---|---|---|---|---|---|---|---|---|
| | | VR1 | | VIII | | | | | | | | | | |
| | | SEQ | | SEQ | Dia- | Heart | Liver | Liver | Liver | Liver | | Bicep | Gastroc- | Tibialis |
| | VR1_ | ID | VR | ID | phragm | VW | LL | LL | ML | ML | Quads | Femoris | nemius | Anterior |
| variant_id | mutation | NO: | VIII | NO: | Left | Left | Left | Right | Left | Right | Right | Left | Left | Left |
| ATLVT019XX1 | SSTAGGAS | 47128 | SNRR | 44883 | −0.21 | −0.32 | −0.11 | −0.09 | −0.25 | 0.16 | −0.18 | −0.70 | −0.55 | −0.58 |
| | NDNA | | GDFN | | | | | | | | | | | |
| | | | NT | | | | | | | | | | | |
| ATLVT019XX10 | SGTTGGST | 46659 | ENRR | 44864 | 0.73 | 1.17 | 0.93 | 0.78 | 0.45 | 0.89 | 1.40 | 1.84 | 1.72 | 1.45 |
| | NDNT | | GDFN | | | | | | | | | | | |
| | | | NL | | | | | | | | | | | |
| ATLVT019XX11 | SGTTGGSS | 46656 | ENRR | 44864 | 0.73 | 0.60 | 0.17 | 0.32 | 0.09 | 0.15 | 1.31 | 1.26 | 1.27 | 1.17 |
| | NDNT | | GDFN | | | | | | | | | | | |
| | | | NL | | | | | | | | | | | |
| ATLVT019XX12 | SSTAGGAS | 47128 | ENRR | 44864 | 1.76 | 1.32 | −0.05 | 1.73 | 1.45 | 1.97 | 1.90 | 1.69 | 2.05 | 1.67 |
| | NDNA | | GDFN | | | | | | | | | | | |
| | | | NL | | | | | | | | | | | |
| ATLVT019XX13 | SSTAGGAT | 47131 | SAQR | 44882 | −1.89 | −0.42 | 0.70 | 0.73 | 0.09 | −0.08 | −2.74 | −2.15 | −0.18 | −1.90 |
| | NDNA | | GDLL | | | | | | | | | | | |
| | | | LS | | | | | | | | | | | |
| ATLVT019XX14 | SGTTGGST | 46659 | SAQR | 44882 | −1.07 | −0.77 | −0.48 | −0.31 | −0.42 | −0.56 | −1.41 | −0.97 | −0.95 | −1.47 |
| | NDNT | | GDLL | | | | | | | | | | | |
| | | | LS | | | | | | | | | | | |
| ATLVT019XX15 | SGTTGGSS | 46656 | SAQR | 44882 | −1.80 | −0.72 | −0.03 | −0.17 | 0.06 | 0.42 | −1.41 | −0.56 | −0.94 | −1.44 |
| | NDNT | | GDLL | | | | | | | | | | | |
| | | | LS | | | | | | | | | | | |
| ATLVT019XX16 | SSTAGGAS | 47128 | SAQR | 44882 | 0.34 | 0.10 | 2.08 | 1.47 | 1.49 | 1.53 | 1.16 | 0.66 | 0.79 | −0.19 |
| | NDNA | | GDLL | | | | | | | | | | | |
| | | | LS | | | | | | | | | | | |
| ATLVT019XX17 | SSTAGGAT | 47131 | ENRR | 44866 | 0.49 | 1.11 | 1.08 | 0.85 | 0.67 | 0.80 | 1.25 | 1.36 | 0.88 | 1.12 |
| | NDNA | | GDFQ | | | | | | | | | | | |
| | | | NT | | | | | | | | | | | |
| ATLVT019XX18 | SGTTGGST | 46659 | ENRR | 44866 | 0.88 | 0.60 | −0.50 | −0.45 | −0.60 | −0.26 | 1.33 | 1.13 | 0.94 | 0.73 |
| | NDNT | | GDFQ | | | | | | | | | | | |
| | | | NT | | | | | | | | | | | |
| ATLVT019XX19 | SGTTGGSS | 46656 | ENRR | 44866 | 1.68 | 1.07 | −0.07 | −0.23 | −0.16 | −0.18 | 1.23 | 1.05 | 1.51 | 0.95 |
| | NDNT | | GDFQ | | | | | | | | | | | |
| | | | NT | | | | | | | | | | | |
| ATLVT019XX2 | SGTAGGAS | 46554 | SNRR | 44883 | −0.17 | −0.01 | 0.63 | 0.80 | 0.78 | 1.01 | −0.18 | −0.67 | −0.65 | −0.72 |
| | NDNT | | GDFN | | | | | | | | | | | |
| | | | NT | | | | | | | | | | | |
| ATLVT019XX20 | SSTAGGAS | 47128 | ENRR | 44866 | 0.62 | −0.59 | 1.32 | 1.24 | 1.35 | 1.34 | 0.49 | 0.31 | 0.53 | 0.51 |
| | NDNA | | GDFQ | | | | | | | | | | | |
| | | | NT | | | | | | | | | | | |
| ATLVT019XX21 | SSTAGGAT | 47131 | SAQR | 44912 | −0.09 | 0.10 | 1.27 | 1.43 | 1.08 | 1.38 | 0.00 | 0.25 | 0.11 | −0.02 |
| | NDNA | | GDRQ | | | | | | | | | | | |
| | | | GI | | | | | | | | | | | |
| ATLVT019XX23 | SGTTGGSS | 46656 | SAQR | 44912 | 0.32 | 0.60 | 1.24 | 1.00 | 1.22 | 1.29 | 0.39 | 0.83 | 0.57 | 0.58 |
| | NDNT | | GDRQ | | | | | | | | | | | |
| | | | GI | | | | | | | | | | | |
| ATLVT019XX24 | SSTAGGAS | 47128 | SAQR | 44912 | 0.87 | 0.77 | 1.66 | 1.20 | 1.50 | 1.29 | 1.06 | 0.68 | 0.80 | 0.77 |
| | NDNA | | GDRQ | | | | | | | | | | | |
| | | | GI | | | | | | | | | | | |
| ATLVT019XX25 | SSTAGGAT | 47131 | SAQR | 44918 | 1.02 | 1.19 | 1.57 | 1.65 | 1.51 | 1.71 | 1.06 | 1.19 | 0.81 | 1.05 |
| | NDNA | | GDRG | | | | | | | | | | | |
| | | | VV | | | | | | | | | | | |
| ATLVT019XX26 | SGTTGGST | 46659 | SAQR | 44918 | 1.26 | 1.40 | 0.62 | 1.47 | 0.89 | 1.18 | 1.28 | 1.19 | 1.19 | 1.13 |
| | NDNT | | GDRG | | | | | | | | | | | |
| | | | VV | | | | | | | | | | | |
| ATLVT019XX27 | SGTTGGSS | 46656 | SAQR | 44918 | 1.66 | 1.35 | 0.59 | 0.64 | 0.48 | 0.65 | 1.08 | 1.41 | 1.40 | 1.23 |
| | NDNT | | GDRG | | | | | | | | | | | |
| | | | VV | | | | | | | | | | | |
| ATLVT019XX28 | SSTAGGAS | 47128 | SAQR | 44918 | 0.69 | 0.62 | 1.66 | 1.71 | 1.62 | 1.66 | 0.25 | 0.54 | 0.39 | 0.37 |
| | NDNA | | GDRG | | | | | | | | | | | |
| | | | VV | | | | | | | | | | | |
| ATLVT019XX29 | SSTAGGAT | 47131 | SAQR | 44919 | 0.07 | 0.23 | 0.02 | −0.49 | −0.06 | −0.12 | 0.00 | 0.12 | −0.10 | −0.11 |
| | NDNA | | GDHV | | | | | | | | | | | |
| | | | NL | | | | | | | | | | | |
| ATLVT019XX3 | SGTSGGST | 46609 | SNRR | 44883 | −0.40 | −0.13 | −0.84 | −0.96 | −0.79 | −0.60 | −0.59 | −0.70 | −0.71 | −0.74 |
| | NDNA | | GDFN | | | | | | | | | | | |
| | | | NT | | | | | | | | | | | |
| ATLVT019XX30 | SGTTGGST | 46659 | SAQR | 44919 | 0.05 | −0.13 | −1.01 | −1.00 | −0.94 | −1.21 | −0.95 | −0.78 | −0.78 | −0.55 |
| | NDNT | | GDHV | | | | | | | | | | | |
| | | | NL | | | | | | | | | | | |
| ATLVT019XX31 | SGTTGGSS | 46656 | SAQR | 44919 | −0.47 | 0.63 | 0.44 | 0.19 | 0.44 | 0.54 | −0.60 | 0.47 | 0.21 | 0.30 |
| | NDNT | | GDHV | | | | | | | | | | | |
| | | | NL | | | | | | | | | | | |
| ATLVT019XX32 | SSTAGGAS | 47128 | SAQR | 44919 | 0.96 | 0.60 | 1.80 | 1.62 | 1.46 | 1.33 | 1.09 | 0.10 | 0.77 | 0.51 |
| | NDNA | | GDHV | | | | | | | | | | | |
| | | | NL | | | | | | | | | | | |
| ATLVT019XX4 | SGTTGGST | 46659 | SNRR | 44883 | −0.08 | 0.57 | −1.10 | −1.79 | −1.75 | −1.38 | −0.12 | −0.23 | 0.12 | −0.46 |
| | NDNT | | GDFN | | | | | | | | | | | |
| | | | NT | | | | | | | | | | | |
| ATLVT019XX41 | SGTSGGAS | 46600 | SAQ- | 48388 | −1.72 | −1.12 | −1.73 | −1.57 | −1.55 | −2.16 | −1.27 | −0.99 | −0.64 | −0.84 |
| | NDNA | | ---- | | | | | | | | | | | |
| | | | -- | | | | | | | | | | | |
| ATLVT019XX44 | SGTSGGST | 46609 | SAQ- | 48388 | −0.75 | −1.18 | −1.79 | −1.82 | −1.75 | −1.62 | −1.20 | −1.11 | −1.30 | −1.10 |
| | NDNA | | ---- | | | | | | | | | | | |
| | | | -- | | | | | | | | | | | |
| ATLVT019XX45 | SGTTGGAS | 46650 | SAQ- | 48388 | −0.92 | −1.04 | −2.05 | −2.19 | −2.29 | −2.44 | −1.19 | −1.07 | −0.98 | −0.99 |
| | NDNT | | ---- | | | | | | | | | | | |
| | | | -- | | | | | | | | | | | |
| ATLVT019XX46 | SGTTGGAT | 46653 | SAQ- | 48388 | −0.82 | −1.68 | −5.37 | −5.35 | −3.65 | −4.45 | −1.31 | −1.37 | −1.37 | −1.08 |
| | NDNT | | ---- | | | | | | | | | | | |
| | | | -- | | | | | | | | | | | |
| ATLVT019XX47 | SGTTGGSS | 46656 | SAQ- | 48388 | −0.72 | −1.17 | −4.53 | −2.59 | −3.50 | −3.93 | −1.21 | −1.93 | −1.13 | −1.73 |
| | NDNT | | ---- | | | | | | | | | | | |
| | | | -- | | | | | | | | | | | |
| ATLVT019XX48 | SGTTGGST | 46659 | SAQ- | 48388 | 0.05 | −0.92 | −4.28 | −2.97 | −3.05 | −2.83 | −0.63 | −1.09 | −0.88 | −0.54 |
| | NDNT | | ---- | | | | | | | | | | | |
| | | | -- | | | | | | | | | | | |
| ATLVT019XX49 | SSTAGGAS | 47128 | SAQ- | 48388 | 0.56 | −0.47 | −1.79 | −1.65 | −1.85 | −2.41 | 0.01 | −0.51 | −0.69 | 0.02 |
| | NDNA | | ---- | | | | | | | | | | | |
| | | | -- | | | | | | | | | | | |
| ATLVT019XX5 | SSTAGGAT | 47131 | ENRR | 44880 | 2.12 | 1.16 | 0.09 | −0.22 | −0.16 | 0.01 | 1.31 | 1.76 | 1.24 | 1.19 |
| | NDNA | | GDFN | | | | | | | | | | | |
| | | | NT | | | | | | | | | | | |
| ATLVT019XX50 | SSTAGGAT | 47131 | SAQ- | 48388 | −0.21 | −1.18 | −1.53 | −1.09 | −1.56 | −2.04 | −0.41 | −1.08 | −1.06 | −1.53 |
| | NDNA | | ---- | | | | | | | | | | | |
| | | | -- | | | | | | | | | | | |
| ATLVT019XX51 | SSTAGGSS | 47134 | SAQ- | 48388 | −0.65 | −0.76 | −0.89 | −1.27 | −1.19 | −1.56 | −0.49 | −0.46 | −0.55 | −0.33 |
| | NDNA | | ---- | | | | | | | | | | | |
| | | | -- | | | | | | | | | | | |
| ATLVT019XX52 | SSTAGGST | 47137 | SAQ- | 48388 | −0.91 | −1.39 | −1.44 | −1.53 | −1.51 | −1.69 | −0.97 | −1.36 | −1.44 | −1.06 |
| | NDNA | | ---- | | | | | | | | | | | |
| | | | -- | | | | | | | | | | | |
| ATLVT019XX53 | NSTSGGAS | 46026 | SAQ- | 48388 | −1.60 | −1.57 | −1.66 | −1.73 | −1.61 | −1.82 | −1.55 | −1.82 | −1.20 | −0.82 |
| | NDNH | | ---- | | | | | | | | | | | |
| | | | -- | | | | | | | | | | | |
| ATLVT019XX55 | NSTSGGSS | 46031 | SAQ- | 48388 | −0.59 | −1.10 | 0.25 | 0.34 | 0.17 | 0.25 | −0.83 | −1.18 | −1.10 | −0.52 |
| | NDNH | | ---- | | | | | | | | | | | |
| | | | -- | | | | | | | | | | | |
| ATLVT019XX56 | NSTSGGST | 46034 | SAQ- | 48388 | −1.17 | −0.86 | 0.79 | 0.76 | 0.63 | 0.85 | −1.14 | −0.68 | −0.75 | −0.31 |
| | NDNH | | ---- | | | | | | | | | | | |
| | | | -- | | | | | | | | | | | |
| ATLVT019XX57 | SGQTGGAS | 46505 | SAQ- | 48388 | −1.39 | −1.22 | −3.42 | −3.18 | −3.61 | −3.46 | −1.87 | −2.07 | −1.40 | −1.83 |
| | NDNH | | ---- | | | | | | | | | | | |
| | | | -- | | | | | | | | | | | |
| ATLVT019XX58 | SGQTGGAT | 46508 | SAQ- | 48388 | −0.36 | −0.87 | 1.23 | 1.20 | 1.11 | 1.05 | −0.80 | −1.29 | −1.46 | −1.05 |
| | NDNH | | ---- | | | | | | | | | | | |
| | | | -- | | | | | | | | | | | |
| ATLVT019XX59 | SGQTGGSS | 46511 | SAQ- | 48388 | −1.21 | −2.42 | −9.13 | −5.27 | −7.76 | −6.52 | −1.80 | −2.91 | −1.96 | −2.08 |
| | NDNH | | ---- | | | | | | | | | | | |
| | | | -- | | | | | | | | | | | |
| ATLVT019XX6 | SGTTGGST | 46659 | ENRR | 44880 | 1.58 | 0.99 | 0.19 | −0.42 | 0.22 | −0.18 | 0.96 | 1.62 | 1.18 | 1.08 |
| | NDNT | | GDFN | | | | | | | | | | | |
| | | | NT | | | | | | | | | | | |
| ATLVT019XX60 | SGQTGGST | 46514 | SAQ- | 48388 | −2.28 | −1.23 | −4.51 | −2.68 | −1.32 | −4.18 | −3.35 | −3.67 | −2.14 | −1.80 |
| | NDNH | | ---- | | | | | | | | | | | |
| | | | -- | | | | | | | | | | | |
| ATLVT019XX61 | NSTSGGSS | 48388 | ENRR | 44859 | 1.54 | 1.51 | 1.81 | 1.82 | 1.78 | 1.84 | 1.76 | 1.44 | 1.21 | 1.34 |
| | NDNA | | GDYN | | | | | | | | | | | |
| | | | SL | | | | | | | | | | | |
| ATLVT019XX62 | NSTSGGSS | 48388 | ENRR | 44866 | 0.80 | 1.05 | 2.16 | 2.09 | 2.04 | 2.25 | 1.18 | 1.14 | 0.84 | 0.88 |
| | NDNA | | GDFQ | | | | | | | | | | | |
| | | | NT | | | | | | | | | | | |
| ATLVT019XX63 | NSTSGAST | 48390 | ENRR | 44859 | −0.74 | −1.32 | −2.38 | −2.32 | −2.51 | −2.24 | −0.52 | −1.07 | −1.26 | −1.12 |
| | NDNA | | GDYN | | | | | | | | | | | |
| | | | SL | | | | | | | | | | | |
| ATLVT019XX64 | NSTSGAST | 48390 | ENRR | 44866 | 0.42 | 0.35 | 0.35 | 0.40 | 0.21 | 0.40 | 1.11 | 0.86 | 0.65 | 0.67 |
| | NDNA | | GDFQ | | | | | | | | | | | |
| | | | NT | | | | | | | | | | | |
| ATLVT019XX65 | NSTSGGSS | 48388 | SAQR | 44912 | −0.64 | −0.55 | 2.01 | 2.09 | 2.03 | 2.15 | −0.47 | −0.27 | −0.42 | −0.18 |
| | NDNA | | GDRQ | | | | | | | | | | | |
| | | | GI | | | | | | | | | | | |
| ATLVT019XX66 | NSTSGAST | 48390 | SAQR | 44912 | 0.72 | −0.20 | 0.25 | 0.41 | 0.29 | 0.43 | 0.57 | 0.27 | −0.01 | 0.21 |
| | NDNA | | GDRQ | | | | | | | | | | | |
| | | | GI | | | | | | | | | | | |
| ATLVT019XX67 | NSTSGGSS | 48388 | ENRR | 44864 | 1.03 | 0.83 | 2.23 | 2.29 | 2.23 | 2.35 | 1.52 | 1.59 | 1.40 | 1.52 |
| | NDNA | | GDFN | | | | | | | | | | | |
| | | | NL | | | | | | | | | | | |
| ATLVT019XX68 | NSTSGGSS | 48388 | SAQR | 44918 | −0.49 | −0.51 | 2.10 | 2.09 | 2.03 | 2.22 | −0.43 | −0.52 | −0.59 | −0.26 |
| | NDNA | | GDRG | | | | | | | | | | | |
| | | | VV | | | | | | | | | | | |
| ATLVT019XX69 | NSTSGAST | 48390 | ENRR | 44864 | 0.72 | −0.25 | −0.18 | −0.29 | −0.15 | −0.47 | 1.17 | 0.40 | 0.46 | 0.64 |
| | NDNA | | GDFN | | | | | | | | | | | |
| | | | NL | | | | | | | | | | | |
| ATLVT019XX7 | SGTTGGSS | 46656 | ENRR | 44880 | 1.79 | 1.28 | 0.62 | 0.60 | 0.37 | 0.37 | 1.49 | 1.48 | 1.52 | 1.65 |
| | NDNT | | GDFN | | | | | | | | | | | |
| | | | NT | | | | | | | | | | | |
| ATLVT019XX70 | NSTSGAST | 48390 | SAQR | 44918 | 2.07 | 1.79 | 1.01 | 0.82 | 0.79 | 1.29 | 1.91 | 1.71 | 2.50 | 1.98 |
| | NDNA | | GDRG | | | | | | | | | | | |
| | | | VV | | | | | | | | | | | |
| ATLVT019XX71 | NSTSGGSS | 48388 | SAQR | 44919 | −0.11 | −0.16 | 1.55 | 1.42 | 1.38 | 1.16 | −0.19 | −0.53 | −0.46 | −0.30 |
| | NDNA | | GDHV | | | | | | | | | | | |
| | | | NL | | | | | | | | | | | |
| ATLVT019XX72 | NSTSGAST | 48390 | SAQR | 44919 | 0.01 | 0.01 | −0.21 | 0.05 | −0.22 | −0.21 | 0.02 | −0.01 | −0.29 | −0.12 |
| | NDNA | | GDHV | | | | | | | | | | | |
| | | | NL | | | | | | | | | | | |
| ATLVT019XX8 | SSTAGGAS | 47128 | ENRR | 44880 | 1.07 | 0.65 | 1.24 | 0.62 | 1.02 | 1.04 | −0.18 | 0.48 | 0.63 | 0.65 |
| | NDNA | | GDFN | | | | | | | | | | | |
| | | | NT | | | | | | | | | | | |
| ATLVT019XX9 | SSTAGGAT | 47131 | ENRR | 44864 | 0.95 | 0.57 | 0.08 | 0.09 | 0.00 | 0.06 | 1.33 | 1.07 | 1.00 | 0.82 |
| | NDNA | | GDFN | | | | | | | | | | | |
| | | | NL | | | | | | | | | | | |
| ATLVT019XXAAV9 | NSTSGGSS | 48388 | SAQ- | 48388 | −0.14 | −0.92 | 0.87 | 0.78 | 0.64 | 0.86 | −0.62 | −1.10 | −1.15 | −0.89 |
| | NDNA | | ---- | | | | | | | | | | | |
| | | | -- | | | | | | | | | | | |
| ATLVT019XXAAV9deco1 | NSTSGGSS | 48388 | SAQR | 44882 | −0.11 | 0.48 | 2.18 | 2.10 | 1.99 | 2.13 | −0.05 | 0.01 | −0.17 | −0.04 |
| | NDNA | | GDLL | | | | | | | | | | | |
| | | | LS | | | | | | | | | | | |
| ATLVT019XXAAV9_1000 | NSTSGGSS | 48388 | ENRR | 44880 | 0.99 | 1.40 | 2.93 | 2.78 | 2.80 | 2.82 | 1.02 | 1.36 | 0.91 | 1.18 |
| | NDNA | | GDFN | | | | | | | | | | | |
| | | | NT | | | | | | | | | | | |
| ATLVT019XXMut1_1000 | NSTSGAST | 48390 | ENRR | 44880 | 1.12 | 0.13 | −0.04 | 0.06 | 0.04 | 0.02 | 1.04 | 0.84 | 0.82 | 0.81 |
| | NDNA | | GDFN | | | | | | | | | | | |
| | | | NT | | | | | | | | | | | |

VW = ventricle wall,
LL = lateral lobe,
ML = median lobe

Overall, this data showed that a subset of the modified AAV capsids from Table 22 tested in NHPs had reduced liver tropism and increased muscle tropism. Modified AAV capsids with “variant-ids” ATLVT019XX10 to ATLVT019XX20 exhibited the greatest reductions in liver tropism and the greatest increases in muscle tropism (or at least not effecting significant reduction in muscle tropism).

DNA samples are analyzed for biodistribution of vector genomes in the liver and quadriceps tissue using NGS. All analytical work is conducted, using an analytical method developed and qualified by that laboratory. Samples were collected at 4 weeks pre-dose and on day 28 following administration.

### 8.25.2. AAV^{-mini} Library in Mice

### Experimental Design

A total of 5 C57BL/6 mice were used for this study according to the experimental design in Table 23. Mice were administered with the indicated amounts or concentrations of AAV^{-mini} library (comprising the 116 modified capsid proteins of Table 20) by intravenous tail vein injection.

TABLE 23
Experimental design for mouse study.

| | | | | | Study end |
|---|---|---|---|---|---|
| Group | | Dose | Dose | Dose (vg/ | point (necropsy |
| No. | Treatment | Route | Vol (ml) | animal or kg) | day 28) |
| 1 | Mini- | IV | 10.0 | 5E13 vg/kg | 5 |
| | Library | | | | |

The mice were sacrificed 28 days after the injection. Individual tissues, notably the liver, heart, and diaphragm, were collected at the time of necropsy. Tissue were immediately placed into the preservative RNAlater, after which the RNAlater was removed and the tissue flash frozen. RNA was isolated from the tissue samples and sequenced to identify the AAVs (AAVs from AAV^{-mini} library, each AAV containing a unique targeting peptide in VR VIII and a unique peptide segment in VR I) localized in each tissue. Using the sequence data, enrichment scores for each tissue were calculated for each AAV (calculated as described in Example 11). After determining a sequence count from sequence read data (FASTQ) following sequencing, AAV variants were counted and normalized. The AAV variants were assigned tissue enrichment scores for each individual tissue analyzed (i.e., heart, liver, diaphragm, and quadriceps). Tissue enrichment data for the AAV^{-mini} library in mice is shown in Table 24 with tissue enrichment scores in columns F-H for the respective tissues.

TABLE 24

| | | | | VR VIII | | | |
|---|---|---|---|---|---|---|---|
| | | VRI SEQ | VR8_ | SEQ ID | | | |
| variant_id | VR1_mutation | ID NO: | insertion | NO: | Diaphragm | Heart | Liver |
| ATLVT019XX1 | SSTAGGASNDNA | 47128 | SNRRGDFNNT | 44883 | −0.56 | −0.10 | −0.08 |
| ATLVT019XX10 | SGTTGGSTNDNT | 46659 | ENRRGDFNNL | 44864 | 0.03 | 0.48 | 0.05 |
| ATLVT019XX11 | SGTTGGSSNDNT | 46656 | ENRRGDFNNL | 44864 | −0.31 | 0.09 | −0.24 |
| ATLVT019XX12 | SSTAGGASNDNA | 47128 | ENRRGDFNNL | 44864 | 1.55 | 1.68 | 1.73 |
| ATLVT019XX13 | SSTAGGATNDNA | 47131 | SAQRGDLLLS | 44882 | −0.51 | 0.49 | 0.09 |
| ATLVT019XX14 | SGTTGGSTNDNT | 46659 | SAQRGDLLLS | 44882 | −0.69 | 0.27 | −0.33 |
| ATLVT019XX15 | SGTTGGSSNDNT | 46656 | SAQRGDLLLS | 44882 | −0.48 | 0.40 | −0.36 |
| ATLVT019XX16 | SSTAGGASNDNA | 47128 | SAQRGDLLLS | 44882 | 0.85 | 1.14 | 2.03 |
| ATLVT019XX17 | SSTAGGATNDNA | 47131 | ENRRGDFQNT | 44866 | 0.29 | 0.74 | 0.08 |
| ATLVT019XX18 | SGTTGGSTNDNT | 46659 | ENRRGDFQNT | 44866 | 0.02 | 0.38 | −0.41 |
| ATLVT019XX19 | SGTTGGSSNDNT | 46656 | ENRRGDFQNT | 44866 | 1.04 | 1.20 | 0.65 |
| ATLVT019XX2 | SGTAGGASNDNT | 46554 | SNRRGDFNNT | 44883 | −0.46 | −0.15 | −0.01 |
| ATLVT019XX20 | SSTAGGASNDNA | 47128 | ENRRGDFQNT | 44866 | 0.29 | 0.58 | 1.52 |
| ATLVT019XX21 | SSTAGGATNDNA | 47131 | SAQRGDRQGI | 44912 | 0.54 | 0.81 | 1.46 |
| ATLVT019XX23 | SGTTGGSSNDNT | 46656 | SAQRGDRQGI | 44912 | 0.37 | 0.51 | 0.46 |
| ATLVT019XX24 | SSTAGGASNDNA | 47128 | SAQRGDRQGI | 44912 | 0.54 | 0.78 | 1.34 |
| ATLVT019XX25 | SSTAGGATNDNA | 47131 | SAQRGDRGVV | 44918 | 0.79 | 1.12 | 1.04 |
| ATLVT019XX26 | SGTTGGSTNDNT | 46659 | SAQRGDRGVV | 44918 | −0.24 | 0.02 | 0.87 |
| ATLVT019XX27 | SGTTGGSSNDNT | 46656 | SAQRGDRGVV | 44918 | −0.63 | 0.11 | 0.33 |
| ATLVT019XX28 | SSTAGGASNDNA | 47128 | SAQRGDRGVV | 44918 | 1.84 | 3.25 | 2.37 |
| ATLVT019XX29 | SSTAGGATNDNA | 47131 | SAQRGDHVNL | 44919 | 2.06 | 0.60 | 0.45 |
| ATLVT019XX3 | SGTSGGSTNDNA | 46609 | SNRRGDFNNT | 44883 | −0.53 | −0.06 | −0.50 |
| ATLVT019XX30 | SGTTGGSTNDNT | 46659 | SAQRGDHVNL | 44919 | 0.62 | 0.10 | −0.14 |
| ATLVT019XX31 | SGTTGGSSNDNT | 46656 | SAQRGDHVNL | 44919 | 0.80 | 0.48 | 0.05 |
| ATLVT019XX32 | SSTAGGASNDNA | 47128 | SAQRGDHVNL | 44919 | 0.45 | 0.68 | 1.23 |
| ATLVT019XX4 | SGTTGGSTNDNT | 46659 | SNRRGDFNNT | 44883 | −0.03 | 0.40 | −0.31 |
| ATLVT019XX41 | SGTSGGASNDNA | 46600 | SAQ------- | 48388 | 0.27 | −0.09 | −0.71 |
| ATLVT019XX44 | SGTSGGSTNDNA | 46609 | SAQ------- | 48388 | −0.55 | −0.87 | −0.95 |
| ATLVT019XX45 | SGTTGGASNDNT | 46650 | SAQ------- | 48388 | −0.76 | −1.16 | −1.86 |
| ATLVT019XX46 | SGTTGGATNDNT | 46653 | SAQ------- | 48388 | −0.71 | −1.17 | −2.03 |
| ATLVT019XX47 | SGTTGGSSNDNT | 46656 | SAQ------- | 48388 | −0.78 | −1.20 | −2.01 |
| ATLVT019XX48 | SGTTGGSTNDNT | 46659 | SAQ------- | 48388 | 0.05 | −0.33 | −1.04 |
| ATLVT019XX49 | SSTAGGASNDNA | 47128 | SAQ------- | 48388 | 1.33 | −0.04 | −0.51 |
| ATLVT019XX5 | SSTAGGATNDNA | 47131 | ENERGDFNNT | 44880 | 1.21 | 0.55 | 0.25 |
| ATLVT019XX50 | SSTAGGATNDNA | 47131 | SAQ------- | 48388 | −0.37 | −0.61 | −0.22 |
| ATLVT019XX51 | SSTAGGSSNDNA | 47134 | SAQ------- | 48388 | −0.53 | −0.81 | −0.93 |
| ATLVT019XX52 | SSTAGGSTNDNA | 47137 | SAQ------- | 48388 | −0.58 | −0.76 | −0.10 |
| ATLVT019XX53 | NSTSGGASNDNH | 46026 | SAQ------- | 48388 | −0.72 | −1.07 | −1.21 |
| ATLVT019XX55 | NSTSGGSSNDNH | 46031 | SAQ------- | 48388 | −0.76 | −0.92 | −0.05 |
| ATLVT019XX56 | NSTSGGSTNDNH | 46034 | SAQ------- | 48388 | −0.52 | −0.53 | 1.15 |
| ATLVT019XX57 | SGQTGGASNDNH | 46505 | SAQ------- | 48388 | −0.92 | −1.22 | −1.24 |
| ATLVT019XX58 | SGQTGGATNDNH | 46508 | SAQ------- | 48388 | −0.31 | −0.40 | 2.01 |
| ATLVT019XX59 | SGQTGGSSNDNH | 46511 | SAQ------- | 48388 | −1.34 | −1.63 | −2.09 |
| ATLVT019XX6 | SGTTGGSTNDNT | 46659 | ENRRGDFNNT | 44880 | 0.57 | 0.34 | −0.16 |
| ATLVT019XX60 | SGQTGGSTNDNH | 46514 | SAQ------- | 48388 | −0.12 | −0.31 | 0.25 |
| ATLVT019XX61 | NSTSGGSSNDNA | 48388 | ENRRGDYNSL | 44859 | 1.70 | 0.48 | 0.89 |
| ATLVT019XX62 | NSTSGGSSNDNA | 48388 | ENRRGDFQNT | 44866 | 0.21 | 0.72 | 1.42 |
| ATLVT019XX63 | NSTSGASTNDNA | 48390 | ENRRGDYNSL | 44859 | −1.17 | −0.92 | −1.40 |
| ATLVT019XX64 | NSTSGASTNDNA | 48390 | ENRRGDFQNT | 44866 | 0.66 | 0.32 | −0.31 |
| ATLVT019XX65 | NSTSGGSSNDNA | 48388 | SAQRGDRQGI | 44912 | −0.16 | 0.14 | 2.19 |
| ATLVT019XX66 | NSTSGASTNDNA | 48390 | SAQRGDRQGI | 44912 | 0.26 | 0.48 | 0.23 |
| ATLVT019XX67 | NSTSGGSSNDNA | 48388 | ENRRGDFNNL | 44864 | 0.21 | 0.50 | 1.49 |
| ATLVT019XX68 | NSTSGGSSNDNA | 48388 | SAQRGDRGVV | 44918 | 0.18 | −0.11 | 1.54 |
| ATLVT019XX69 | NSTSGASTNDNA | 48390 | ENRRGDFNNL | 44864 | −0.39 | 0.01 | −0.38 |
| ATLVT019XX7 | SGTTGGSSNDNT | 46656 | ENRRGDFNNT | 44880 | 0.99 | 0.96 | 0.34 |
| ATLVT019XX70 | NSTSGASTNDNA | 48390 | SAQRGDRGVV | 44918 | 1.62 | 1.57 | 1.98 |
| ATLVT019XX71 | NSTSGGSSNDNA | 48388 | SAQRGDHVNL | 44919 | 1.33 | 0.65 | 2.20 |
| ATLVT019XX72 | NSTSGASTNDNA | 48390 | SAQRGDHVNL | 44919 | 0.42 | 0.30 | −0.06 |
| ATLVT019XX8 | SSTAGGASNDNA | 47128 | ENRRGDFNNT | 44880 | 1.24 | 0.97 | 1.79 |
| ATLVT019XX9 | SSTAGGATNDNA | 47131 | ENRRGDFNNL | 44864 | −0.07 | 0.29 | 0.42 |
| ATLVT019XXAAV9 | NSTSGGSSNDNA | 48388 | SAQ------- | 48388 | 0.28 | −0.32 | 1.03 |
| ATLVT019XXAAV9deco1 | NSTSGGSSNDNA | 48388 | SAQRGDLLLS | 44882 | 0.21 | 0.92 | 1.80 |
| ATLVT019XXAAV9_1000 | NSTSGGSSNDNA | 48388 | ENRRGDFNNT | 44880 | 0.59 | 0.57 | 1.76 |
| ATLVT019XXMut1_1000 | NSTSGASTNDNA | 48390 | ENRRGDFNNT | 44880 | 0.29 | 0.25 | −0.12 |

Overall, this data showed that a subset of the modified AAV capsids from Table 25 tested in mice had reduced liver tropism and had increased muscle tropism. Similar to the NHP data, modified AAV capsids with “variant-ids” ATLVT019XX10 to ATLVT019XX20 exhibited the greatest reductions in liver tropism and the greatest increases in muscle tropism (or at least not effecting significant reduction in muscle tropism).

DNA samples are analyzed for biodistribution of vector genomes in the liver and quadriceps tissue using NGS. All analytical work is conducted using an analytical method developed and qualified by that laboratory. Samples were collected at 4 weeks pre-dose and on day 28 following administration.

### 8.26. Example 26: Assessment of AA Vs Having Capsids a Targeting Peptide in VR VIII and a Peptide Segment within VR I in Mice and Non-Human Primates

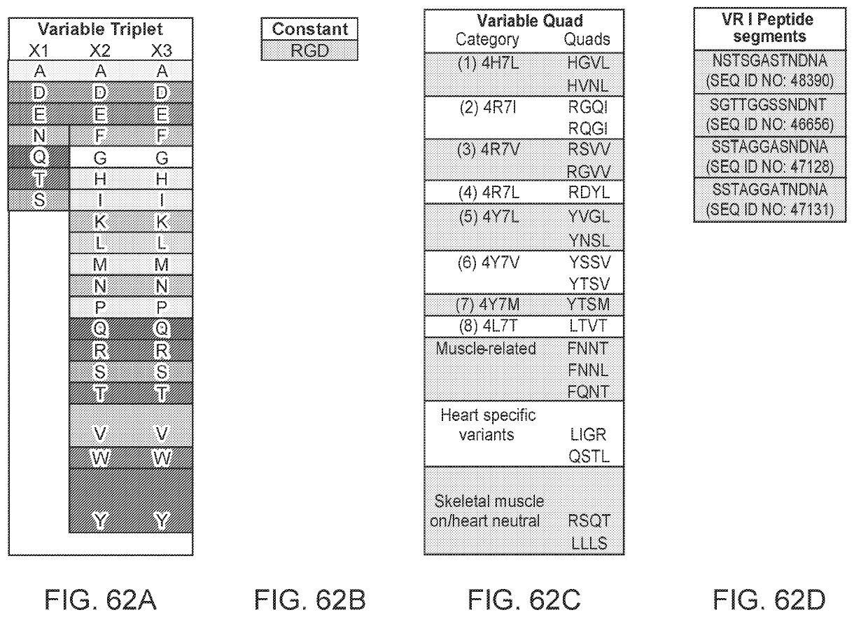

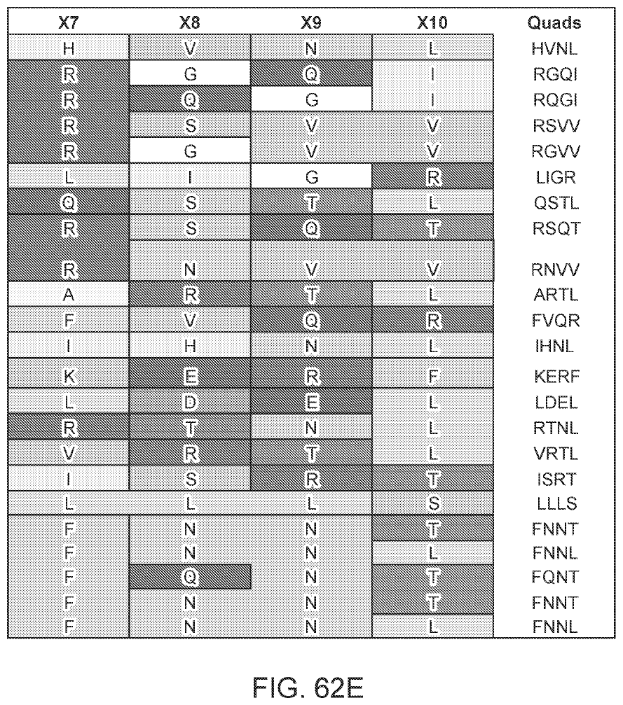

The objective of this study is to assess AA Vs comprising a capsid protein having a targeting peptide in VR VIII and a tropism-altering peptide segment within VR I in non-human primates (NHP) to identify modified AAV capsid proteins with potential for muscle targeting (e.g., cardiac tissue, skeletal muscle, or cardiac tissue and skeletal muscle). Overall, 58,129 modified AAV capsid proteins are tested. The combination of targeting peptides and peptide segments tested in this example are as shown in FIGS. 62A-62E. The targeting peptides include a variable triplet, an RGD, and a variable quad (see FIG. 62A-62E).

In particular, a library containing the 58,129 modified AAV capsid proteins is produced, which is referred to herein as the AAV-Lib3 library (“AAV^{-Lib3} library”). The combinations of targeting peptides and peptide segments included the modified AAV capsid proteins that comprised AAV-^{Lib3} are as described herein. The targeting peptides are positioned in VR VIII of AAV9 between amino acid residues 585 and 589, replacing amino acids 586, 587, and 588. The peptide segments are positioned in VR I. For example, the amino acid sequences for the peptide segments in VR I correspond to the 12 amino acids between S261 and Y274. Controls include AAV9.

Three animals are treated as summarized in Table 25 below. Immunosuppression of the animals begins 7 days prior to vector administration. The animals are administered with the indicated amounts or concentrations of AAV-Lib3 (comprising the about 58,129 modified AAV capsid proteins and controls) by IV.

TABLE 25
Experimental design for non-human
primate study with AAV^{-Lib3} Library

| | | | | | Study end |
|---|---|---|---|---|---|
| Group | | Dose | Dose | Dose (vg/ | point (necropsy |
| No. | Treatment | Route | Vol (ml) | animal or kg) | day 28) |
| 1 | AAV^{-Lib3} | IV | 2.0 | 2E+13 | 3 animals |
| | | | | (3.7E+8 per | |
| | | | | variant) | |

Animals are sacrificed on day 28 after AAV vector administration and their organ samples are collected for analysis. Tissue samples include various muscle tissues and the liver.

DNA samples are analyzed for biodistribution of vector genomes in the muscle tissues and the liver using NGS.

Gene transfer efficacy of each AAV vector is assessed by measuring mRNA transcript in the muscle tissues and the liver using NGS.

Overall, this data shows that a subset of the modified AAV capsid comprising various targeting peptides tested in NHP have increased muscle tropism.

### 8.27. Example 27: Analysis of AAV Capsids Including X₁X₂X₃RGDX₇X₈X₉X₁₀ Targeting Peptides

The objective of this study was to assess the 50,500 modified AAV capsid proteins from AAV^{-Lib2} library in non-human primates (NHP) to identify capsids having targeting peptides that enhance muscle tropism.

In particular, a library containing the 50,500 modified AAV capsid proteins was produced, which was referred to herein as the AAV^{-Lib2} library. The library was constructed as described elsewhere herein. The targeting peptides were positioned in VR VIII of AAV9 between amino acid residues 585 and 589, replacing amino acids 586, 587, and 588. Targeting peptides included the sequences as described in SEQ ID NOs: 48391-98943 (see Table 29), which is hereby incorporated by reference in its entirety). Controls include AAV9. See also FIGS. 62A-62C.

Three animals were treated as summarized in the table below. The animals were administered with the indicated amounts or concentrations of AAV^{-Lib2} (comprising the about 50,500 modified capsid proteins and controls) (see Table 26 below) by IV.

TABLE 26
Experimental design for non-human
primate study with AAV^{-Lib2} Library

| | | | | | Study end |
|---|---|---|---|---|---|
| Group | | Dose | Dose | Dose (vg/ | point (necropsy |
| No. | Treatment | Route | Vol (ml) | animal or kg) | day 28) |
| 1 | AAV^{-Lib2} | IV | 2.0 | 2E+13 | 3 animals |
| | | | | (3.7E+8 per | |
| | | | | variant) | |

Animals were sacrificed on day 28 after AAV vector administration and their organ samples are collected for analysis. Individual muscle tissues, notably the biceps femoris, quadriceps, diaphragm, heart aria, heart ventricle wall, and liver were collected at the time of necropsy. Tissue isolated and analysis was performed as described elsewhere herein.

DNA samples will be analyzed for biodistribution of vector genomes in biceps femoris, quadriceps, diaphragm, heart aria, heart ventricle wall, and liver.

RNA samples were analyzed for vector expression in the each of the six tissue types collected at day 28 (biceps femoris, quadriceps, diaphragm, heart aria, heart ventricle wall, and liver). AAV^{-Lib2} library data was analyzed as described elsewhere herein (see. e.g., Example 17 “AAV VRI (AAV^{-Lib1}) Capsid Libraries”).

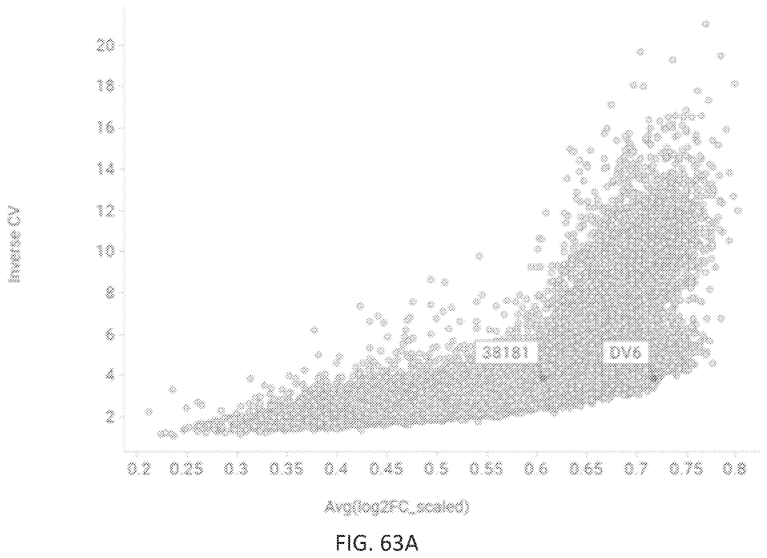

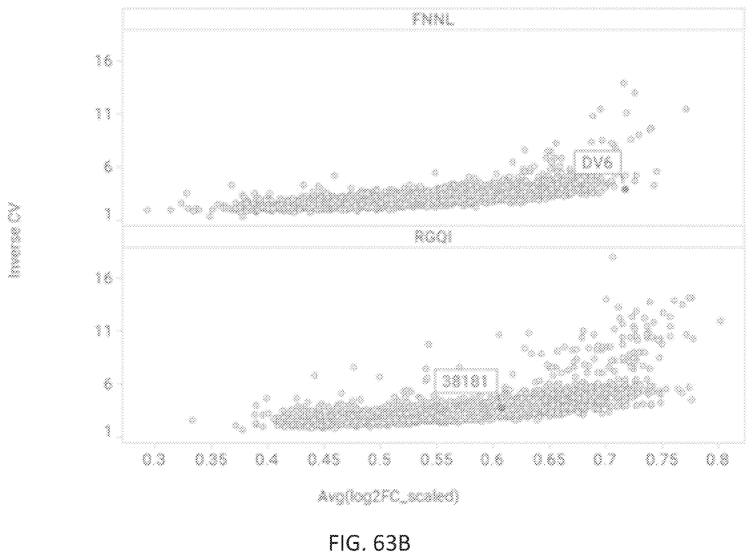

The AAV^{-Lib2} library data (see FIG. 63A) was analyzed to determine how targeting peptides having a particular quad sequence performed. FIG. 63B shows performance of capsids having a targeting peptide where the targeting peptide had a quad selected from FNNL, FNNT, and RGQI.

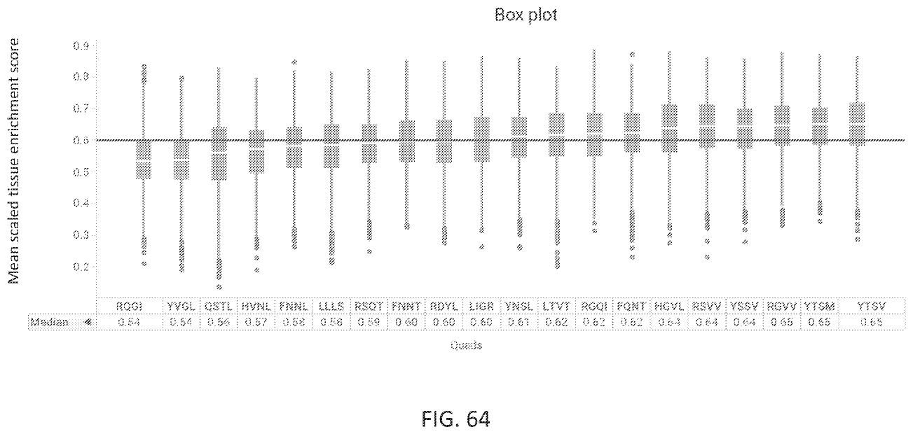

As noted above, AAV^{-Lib2} included targeting peptides having one of 20 different quads. FIG. 64 shows the amount of RNA in muscle tissue for the sum of all the capsids in the AAV^{-Lib2} library having the indicated quad sequences. This analysis revealed that targeting peptides having YTSV, YTSM, RGVV, RSVV, YSSV, and HGVL were the most muscle tropic quads. Targeting peptides having these quad sequences corresponded to X₁X₂X₃RGDYTSV (SEQ ID NO: 98941). X₁X₂X₃RGDYTSM (SEQ ID NO: 98942), X₁X₂X₃RGDRGVV (SEQ ID NO: 98928), X₁X₂X₃RGDRSVV (SEQ ID NO: 98931). X₁X₂X₃RGDYSSV (SEQ ID NO: 98937), and X₁X₂X₃RGDHGVL (SEQ ID NO: 98938), respectively.

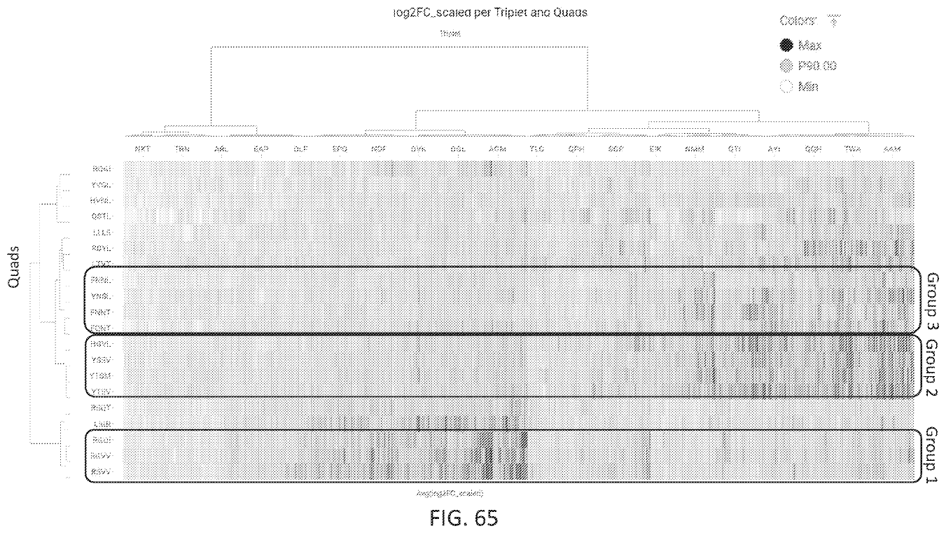

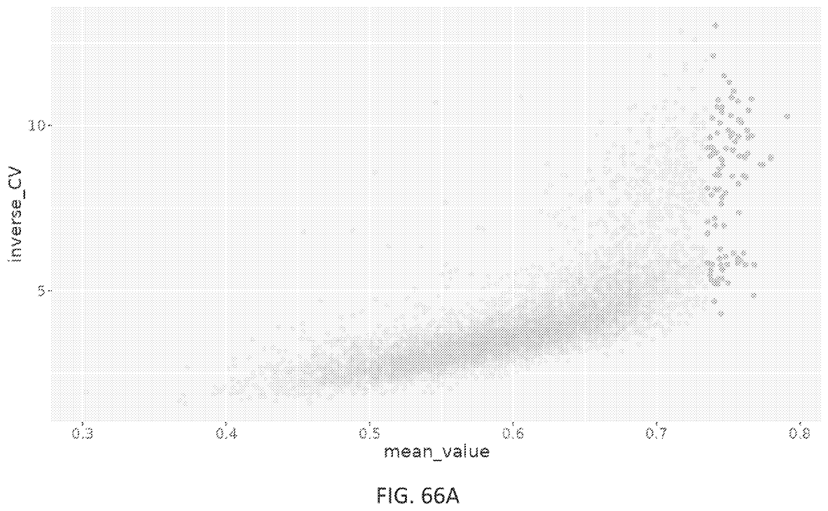

Looking globally at roughly all 50,500 capsids in the AAV^{-Lib2} library FIG. 65 is a heatmap showing enrichment scores averaged across all muscle tissues for each of the capsids in the AAV^{-Lib2} library. Hierarchical clustering of the data revealed at least three groups of capsids performed similarly: Group 1, Group 2, and Group 3 (see FIG. 65). Data associated with FIG. 65 can be found in Table 29. The data show that targeting peptides with quads in group 1 have enhanced muscle tropism when combined with certain X₁X₂X₃, e.g., DII, DWM, EEI, DML, DWI, SLE, EIN, NHE, DFI, EEL, TEQ, TDA, EDT, NEV, TDW, QFE, EDY, DTT, EPL, SEN, SEQ, TAE, EVN, ELN, DVQ, ETI, EVI, ESV, ETW, SEW, DNW, EVF, EAW, EPF, EIY, EIF, EPY, DVI, DMM, DQI, DHL, DTL, DVL, NDL, DLL, DMQ, NEF, DFL, DIM, TEW, DYI, SDY, DYY, DHF, DKE, DTW, DTI, ELY, TEY, TEI, DAI, DQY, DMY, EWG, DMV, DMI, EPH, QEG, DIN, NEI, EYY, DIV, SEG, DVG, DYQ, EGF, NDI, EGY, DVF, DVH, DGF, DIY, DSF, DGW, EHY, DRE, TEH, DTS, NEN, NEM, NEH, TEN, DSN, DVT, DQS, DKD, DTH, DVV, DQK, NET, DKP, TEV, NDS, QET, EVL, SET, SDT, AEQ, QEF, SEY, SEF, SPF, EGQ, ETH, TDQ, QEA, QDQ, AEN, ESS, NDT, EFM, EFI, EHM, DFM, QDT, SME, DYT, EHV, ENV, EAV, EAI, ESI, DAT, ENQ, EAM, ADN, EFQ, SDS, TDH, SDH, DAS, TWE, SSF, DRD, EFL, TDF, QDA, EMH, SGE, AEW, DAH, TET, TDM, TNE, SAE, NSE, SFE, QDI, DSA, ADV, SEI, AEI, QDV, ADT, DNM, DNQ, ADL, TDL, SDL, SDM, TDV, DNI, DHY, DTY, DAA, DSY, QLY, DVM, DAY, DMT, DQT, DAQ, DTV, DSH, QDF, DST, DNL, DSI, DFV, DNY, DAF, DKI, DKF, DTM, DSL, NDV, TDI, DSV, DAV, DKV, DAM, DNV, DKM, DKL, DKW, DSM, ENI, SDI, DKT, QGE, NNE, QYW, AGM, ESH, QDH, QEH, DYH, ENS, DKQ, TGE, DSS, EST, DSQ, DNS, EFV, SWE, DKN, DKS, SEH, EAL, EAS, ADQ, SDQ, ADI, SDV, EAH, ASE, EAF, EYV, DKA, SNE, AGE, EGV, EQF, ETF, EVY, EQY, QEY, QEN, SAD, AEF, EMF, SDF, ADF, ADY, AVF, TLY, SIY, SLY, AIY, DYV, AEY, ENF, EMY, QDY, ETY, EAY, TVY, SVY, QVF, QVY, TMY, DKY, ALY, AVY, TMW, EQW, SPW, SIW, DTQ, TFF, AMF, EYT, DRV, SPY, SIF, SLF, ENY, EWY, EYI, EFY, TLF, TVF, TYY, TFY, SVF, SYW, TAW, SAW, TIY, NIM, NEM, NII, NIV, SII, SIV, SEM, TTI, NSY, SSY, SNY, SEV, AEH, ALT, SGQ, QTY, SYY, AMY, QMY, SGF, SGY, ALF, TTF, SFN, QAF, ASF, SAF, TAF, TAY, TGL, SAY, QAY, ADH, ASI, SSV, SSI, TGV, SGV, DPH, TEG, DLY, ESE, TEF, NPE, DHA, DLI, DWG, EWM, EET, EIL, TEL, DYL, EMW, EVW, EPV, EDV, SLD, EGH, QRY, TDG, SQE, ELH, EYW, EMM, EFF, QEI, EQG, DVS, ESP, EIQ, EPM, EPQ, SPE, EPN, NQD, ELQ, TDT, ELT, EPI, ETM, ETG, EHI, EMI, EIT, ETV, EVV, EVM, TPY, QEW, EIW, EPT, EIH, DFY, DQL, NDA, DIW, TDY, ELF, DVY, ESG, EPG, EIG, ELG, EVG, DHI, EIS, DYM, DFT, DFH, EQS, TPF, EEF, DWN, SDN, EYL, AET, DMN, QSP, DNA, DHM, DHT, EQL ESN, EYF, EHF, NAD, AEG, AES, TEM, SEL, DYS, EVH, EAN, EGL, TFE, ETL, TYE, SHE, ELM, TYD, SYE, ESA, EFT, EAT, AMD, AND, DMS, QMD, SLQ, EYS, QDN, EYQ, EKD, QYD, QFD, DER, AFD, EAA, EFS, DHV, QSE, TSD, SFD, DGY, TWD, QWD, EHL, QGD, QSD, AWD, AGD, QIY, EMQ, EAG, ERD, DDR, DAL, QDL, DNT, EGN, ENH, NGE, DYN, DNH, DNF, ESQ, ESM, ESL, SSD, ENL, EMV, QDS, TSE, SWD, ASD, ENT, SSE, EGM, EGS, EGT, EGI, AIF, NIN, NIQ, NYW, EPW, EGG, TIW, QIW, TIW, EKW, NLY, SVM, TPT, AAW, AIH, TIH, QGT, QFF, QYF, QFY, EHH, QLH, TYF, STY, DRL, EYM, SFF, QLF, QIF, APF, TYW, QUI, QPF, SPI, TIL, SVL, NVI, SIM, SLL, QEL, AEM, AEL, QEV, QDM, NDM, SYL, ASL, TPL, ANI, NMI, TIM, AEV, TMI, SWI, SFI, SNW, TNF, NAF, ASP, QSQ, APY, SFM, TAN, QAW, SGS, SGH, TGF, QSN, ERY, QMN, TGQ, NGM, NGV, AMN, ALN, QFV, AAF, TFV, TSY, EDK, ASN, SAV, QSV, QGV, QST, ASM, TAI, QSM, QSL, QSI, SSL, AGV, TGT, AGI, QGM, TGM, SGM, SGI, THD, QVE, ALE, QLE, EWP, AFP, AEE, ADE, NIE, TYP, QIE, TFP, SYP, ELW, DVW, NEW, EPA, EVA, TLE, AWE, NME, QME, ATE, AYE, DQM, TPD, DFG, QPE, EDI, EWL, ELL, QPD, EWF, ALD, SVD, QVD, ADA, APD, AHE, EQL, DTA, AFE, DAW, QAE, TME, STE, THE, QID, AAE, EIA, EML, SDA, SIE, TQE, TVD, NWE, NOE, EYG, QYP, EIV, EII, QFP, TID, EQN, EVS, TYG, EAQ, ELV, QLD, TLD, QQD, TIN, TLN, SPN, TPN, NFD, NEY, DSW, NMD, DGA, DYA, DIH, DNP, DQW, DQV, EFA, EPS, DFN, DWV, ELI, DTF, EQT, DHW, ESF, DEK, DMH, EWS, EHA, TTD, EWI, QLQ, SQD, QHD, NSD, EMA, TND, ATD, DGS, TAD, TMD, QES, AHD, QAD, DGH, DGL, NTE, ENG, SMD, NDQ, EMS, ENA, EMT, EYN, DDK, EWA, EWQ, EFH, QWE, DGT, DGM, EWV, EWH, EWT, QND, DGV, ESY, AAD, SND, ANE, TIG, QIG, AVD, SFP, AWP, QDW, SLW, TPA, AII, TIV, SWP, AFY, APN, AVN, TVN, TIS, TIQ, TLQ, AIQ, QVN, NFY, QFG, EMN, NVF, SYF, SFW, TPQ, AIL, TWI, QWN, TWN, QWQ, AQF, ASA, TWV, EWN, TMF, NGI, ATN, SAS, TMQ, TMN, AYN, TQF, SMF, QMQ, SMQ, QAN, AAY, SFQ, TYQ, TFQ, TWQ, AVI, and SGL. Targeting peptides with quads in group 2 have enhanced muscle tropism when combined with certain X₁X₂X₃, e.g., APW, TEL, TDA, QPY, SPN, EHY, DWK, DLK, DFK, DVK, NSI, DIR, SPF, SEL, DRT, DRF, ADL, TDL, SDL, DNY, DKI, NDV, DKM, DNH, DNF, DSS, EST, EWT, DKN, DKS, SEH, ESQ, ESL, QND, EAH, AIF, AVF, QVF, TMY, ALY, NNG, NIF, NTF, NFF, AWF, NPY, SWF, AII, AYF, AQW, NFY, AGP, QQF, TKE, TNG, NSF, NAW, QAG, ERG, NKD, QSG, QNG, EAK, QWF, SWY, TFF, TYF, NYY, QFG, NWA, AMF, STY, TNW, ANW, AWM, TSF, DRL, DRV, SPY, NVF, SIF, QLF, SLF, QIF, SYF, APF, AGF, SVF, SAW, TIY, DHK, DAK, EGK, DYK, QNL, QPL, SPV, NPM, STL, NIL, AGA, NTV, SQA, QQA, NNF, NWL, NMA, NNA, NHN, NFH, ELR, NYM, ERA, NFS, SRD, NMM, NNL, NNQ, NNS, QDR, SDK, NNI, AQH, ANA, QNH, QWG, AWA, QWA, EQK, SHS, QFA, SFA, TFA, AYG, EKQ, ERN, EMR, EFR, AYM, EAR, EMK, EYK, EWK, QPI, QPF, NPL, SPI, SPL, NPI, SIV, SLL, SSA, AEM, TML, TLL, SML, TYL, QDM, NSN, AQL, NAT, SQQ, QTI, NAH, SQL, STH, NTT, AEK, ANV, QGL, AGL, QMV, STM, NOM, STI, TQL, SMM, SKE, SQT, SYL, ASL, SLV, NAM, TTV, TPL, TPL, ATL, DNK, DSR, ANL, SHL, SNL, TNL, QNI, QAL, ANI, QNM, QYM, SQL, SNI, QNV, SHV, SHM, QMM, ANM, AHM, SQM, NAL, AQT, EKL, NNM, TNM, SNM, QQI, TQV, TQI THM, SQV, AAV, THL, AQI, NNV, TNN, TNI, SNN, TQM, NAI, TNV, SWT, NMV, NQI NQL, NMI, NSS, SHF, QNF, SWL, AQM, SYV, TFM, SYM, TYM, ATI, TTI, SLI, ALI, AYI, QLV, TMI, SMI, QFI, NMY, NAY, NMN, NMF, ANF, NWT, QWM, TFT, SWM, TWM, QNW, STF, AFM, NSY, SSY, SNY, THY, TNY, QNY, THF, NSV, ATY, SKD, TNF, TWT, QWT, SFS, TFS, DRQ, NYN, DER, EKG, ENN, AWN, QWQ, TQH, ALV, QHQ, DVR, STS, SQS, AAA, QGQ, QSQ, QAQ, SIH, APY, QGN, AGQ, ASQ, AAQ, SQN, NON, ERM, ALH, NYH, TGH, ATH, QTH, TQT, QQH, AQF, QMH, EKN, ERV, ERS, AFT, EKF, EKT, QSA, NFT, QYH, NYS, TSA, QYY, AYY, AHY, QFN, AWQ, NWQ, QAW, NMS, NWN, QMF, AMH, TSN, TAH, TMH, QFH, NWS, SWN, QRD, QKD, AKE, TSS, AYH, TSH, AHQ, ADK, ADR, ASA, ASS, SNS, QSS, SAH, SMH, QAH, AAH, AHN, QNS, QAS, ANS, ANN, SWA, SWS, TWS, ERI, EKS, QWS, AYS, AFS, SYS, QFS, QYN, QFT, AYT, TNS, TYS, SFT, QNQ, QYS, SYT, ANQ, SWV, SNQ, ANY, TNQ, ATF, SSQ, SSN, TWA, SGQ, SSS, TWY, SMY, TGN, TGY, QWY, SQF, TGF, QTY, TTY, AMY, QMY, ERY, TGQ, SGY, TMF, ALN, ARD, ATN, NAS, SAS, AQN, SYG, SYN, TFN, TMN, AYN, SGN, QNN, DKG, AAN, SMN, SAN, TTF, TQF, SFN, QAF, QFV, AAF, ASF, SAF, SMF, ASY, AFN, TAF, TSY, TAY, QMQ, SMQ, TYV, TGL, QAY, AAY, ADH, EDK, ASN, QYQ, SFQ, TYQ, TWQ, SWQ, AYQ, SYQ, AMV, TMV, TAV, AQV, TYT, TAT, QGI, QQV, SAQ, NAV, SAV, ASV, QSV, QAV, QAI, QAM, TAM, DTR, DQR, DMR, NNT, NYT, NMT, NST, ATT, SMV, SNT, QNT, AAT, SAM, SAL, QGV, QST, QYT, ASM, TSM, AMT, TST, ANH, SNV, TNT, ANT, SST, AST, TAI, DAR, SSM, TAL, NSM, TSL, SNF, TSI, TSV, TDK, SAT, SAI, QSL, QSI, ASI, SSL, SSV, SSI, AGI, QGM, TGM, SGM, SGL, SGV, ARL, NWE, SFL, TPY, DRP, DTK, DMK, SEY, SME, EAI, AHD, ADN, EFQ, EMH, SGE, DNQ, DNI, QLY, DGT, EGN, DYN, EWV, ESM, EAL, ASD, DKA, ADF, ADY, SLY, AEY, ETY, EAY, TGA, AVM, TPT, SVI, QSH, TTQ, QTN, TLH, AWY, SLT, AWV, QQY, NFV, SFY, TYY, SHA, TEK, QFK, NNY, EPR, NIT, NHI, NTY, SHY, NFM, NFN, NDK, NER, SER, NDR, QDK, SDR, QNA, TNA, THT, SNH, AYA, NFA, AFA, AHA, QHA, EYR, ENR, EHK, QVL, SVL, NIV, NVI, ADM, SAA, SQH, NQV, ALL, ATM, QYA, STT, DGR, NHT, SHT, QHT, TTL, NHM, QHL, DNR, QHM, QFM, QMI, AHV, THI, AHT, TNH, NHV, THV, SHI, ATV, TWI, NYV, TYI, DYR, SFI, NQF, NWM, NSL, NSP, TQY, DRS, ASP, TKD, QWI, QWN, TWN, QWV, THN, TTM, TQQ, NMQ, TYA, STQ, STN, AGN, EKH, ERT, TFH, SFH, AFH, ENK, NYQ, SFM, QGS, AHF, QLT, QHY, AQY, TRD, ERH, QWH, TYH, TRE, TAQ, AER, TDR, ASH, EKI, AFV, TYN, ALT, NGN, SYY, EWN, NGM, NAN, TAS, TFV, SFV, QAN, SGT, SAY, AFQ, TFQ, QFQ, EFK, SYH, QHV, AMM, THQ, AAI, AAM, NSQ, QAT, TSQ, SHQ, QSM, AGV, AGT, TGT, TGI, AVI, TGV, SGI, ESP, SDA, TPN, EAW, NEQ, DSN, DIK, DNA, QES, SLQ, EKD, SDH, SSF, TET, QDI, DSA, DNM, DKT, DGM, SQY, TLT, QEH, DKQ, TGE, DNS, SDV, SNE, EGT, EGV, AAP, AHG, TWK, TQA, TVT, APQ, SVT, NGT, SSH, NLV, AGY, QFF, QYF, NWV, EKA, EKY, SMG, NAG, ASG, TFI, ELK, SEK, QTL, NML, DHS, ETR, NQQ, QWL, ANP, AHH, NEK, NNN, SNA, EWR, ESR, SHN, SFG, SYA, APV, QPV, SPT, TPQ, QVI, TPV, AVL, SEM, NDM, API, QLM, QTM, QQL, QHI, NFL, AYV, NKE, TIM, QYV, SWI, DRN, AWI, SMA, NRE, QTS, QVV, QQT, QMA, QQS, QAA, ATQ, ERL, TGS, QQQ, AQQ, QHF, TAN, QSY, QSN, NGQ, TWH, TMS, QMS, TQN, TMT, QTV, THS, TTT, SMT, QMT, SMR, ADA, AAE, EPY, EYL, NWI, TSP, DGQ, NDH, QFD, EHL, ERD, DAF, ENI, ENH, QQW, AGM, ENL, EAF, EYV, ENT, SSE, AGE, QEY, DRH, NIQ, SVV, SVM, QPT, TVL, QTQ, SVQ, TIQ, NLQ, SIQ, AWL, EHH, NGY, NHF, NSA, NAA, NTH, QLI, NWH, QRE, AML, EVR, SNP, NHS, THH, NOS, TTS, AHS, EKV, NFQ, APL, QPM, NPV, SIL, TII, STV, QEL, TFL, SIT, NAQ, TMM, AAL, QQM, AMI, QFL, AHI, SYI, SSP, NVV, TTN, TAA, TQS, TMA, ALA, QQN, NSH, TTH, SRE, ERQ, QHS, SWH, SEV, AVT, ALQ, DKH, AMQ, ARE, TWV, NGL, NGV, TMQ, TLV, EER and QTT. Targeting peptides with quads in group 3 have enhanced muscle tropism when combined with certain X₁X₂X₃ e.g., DMK, ATD, EEK, QMD, EFS, ERD, DDR, TDM, SAE, EHS, ENH, SWE, SNE, NNG, QAG, ERG, QSG, QNG, ASG, QFG, AMF, ELR, NFM, NNS, NNI, SDR, EQR, EHR, EWR, EQK, ESR, EKQ, EYR, ENR, EMK, EYK, EHK, EWK, QNI, TNI, TYI, SNY, DRQ, AWI, QWI, DFR, EKG, QYG, QWQ, EKN, EKF, EKT, AFH, ENK, NYS, DKH, AAG, QMF, QFH, QKD, ARE, AHQ, ADK, ADR, AHN, QNS, ANN, SWS, EKS, AFS, QFS, TNQ, TGY, NGL, ARD, AKD, AAF, SMF, AFN, SGT, TGL, ASN, SFQ, AFQ, TFQ, QFQ, EFK, SWQ, AYQ, SYQ, DQR, DMR, ASM, ANT, SHQ, TSL, SNF, QSL, ASI, SGL, SGI, NDA, DHQ, DFK, DTK, DNA, SDS, TDH, DGL, QDV, SDM, DKT, DGM, SQY, DKS, SSD, EMV, TSE, EYV, SSE, EGM, SDF, TTG, QLG, NNP, AGS, QSF, TLM, NGA, AWY, NKD, SWY, TFF, NSG, NGY, QEK, SEK, TEK, AQA, NVL, NHI, TER, NML, NMA, NNA, NHN, ANP, AMS, NER, SER, DKK, NMM, QDK, AHS, TNA, THT, SNH, QNH, ESK, SHS, AYM, SSA, NAT, QLM, QQM, SQI, SNI, QNV, SHV, SHM, NNV, TNN, AHT, TNH, NHV, TQM, THV, SHI, NYL, NMV, NFL, AYV, NOI, NOL, NMI, NSS, SHF, SYM, NYV, AYI, SWI, QFI, ANF, TFT, TWM, QNW, NSY, THY, TNY, NYN, AWN, SMA, NSH, TGH, SFH, SWH, SFM, NFT, QYH, QGS, TGS, TSA, QQQ, QYY, AMQ, TAN, AWH, NMS, TSN, TAH, TMH, QWH, NWS, SWN, QRD, TYH, AKE, TAQ, AER, AYH, ASA, SNS, QSS, SAH, SMH, ASH, QAS, ANS, SYS, QYN, TYN, TNS, SFT, ANQ, SGS, SGH, SSS, TWY, QTY, SYY, NGM, TMS, SYG, TQN, SYN, TFN, TMN, AYN, SGN, QNN, SMN, SAN, QAF, ASF, ASY, TAS, TAF, TFV, SMQ, SFV, TMT, QAN, SAY, EDK, AMV, QHV, THS, TAT, QGI, QQV, NAV, SAV, ASV, QSV, QAV, AAI, AAM, QAM, TAM, DTR, NNT, NYT, NMT, ATT, QTT, TTT, QAT, SMV, SNT, QNT, AAT, SAM, SAL, QGV, QST, QYT, TSM, ANH, SNV, TSQ, TNT, SST, AST, TAI, TSI, TSV, TDK, SAT, QSM, SAI, QSI, SSV, SSI, AGT, TGT, AGI, QGM, TGM, TGV, SGM, SGV, SQE, EHT, EYL, DSG, SFE, QND, TQA, AWV, NFV, TSG, SSG, DRG, ANW, TFI, TSF, QNL, STL, TWL, NNF, NND, NYM, NFI, ETR, SNP, EIR, NFS, NNH, NNL, AHL, NNQ, NDR, NNN, QDR, SDK, SNA, AFG, NYA, QHA, QFA, SHN, NFQ, SYA, TFA, EMR, EFR, EAR, NOM, TTL, TQL, SMM, SKE, SYL, ASL, SLV, TTV, AAL, NHL, NHM, QYL, QHL, ATL, DNR, DSR, ANL, SHL, SNL, TNL, QAL, AMI, ANI, QNM, QYM, QHM, QFM, QMM, QMI, ANM, AHM, SQM, AQT, EKL, NNM, TNM, SNM, QQI, TQV, TQI, THI, QHI, THL, SNN, TNV, SWL, AQM, SYV, TFM, TYM, TWI, AFI, ATI, DYR, QLV, TMI, SYI, SFI, NWM, NWT, NSL, STF, SNW, AFM, SSY, NSV, TNF, NAF, TFS, QWV, ERT, TQT, AFT, NYQ, ERL, NRD, TRD, AWQ, TDR, TSH, ASS, AYT, TYS, QYS, SYT, SWV, SNQ, SMS, NAS, SAS, QFV, SAF, TWQ, TAV, AMM, TYT, QAI, TST, DAR, SSM, TAL, NES, DWK, DLR, DIR, SDT, EAN, ETH, TYD, SYE, EAM, TND, AYD, TSP, DRT, NDH, SSF, EFH, DNQ, DSH, DNT, SDI, DKQ, EWT, EAL, EAS, ESL, ASD, EAH, ASE, EAF, ENT, SIY, QDY, APG, AIG, SSW, NPG, ERP, TVI, QSH, SLS, EDR, AWL, SLH, EQH, EHH, NWV, EKY, STY, DRI, EYH, DRV, SPY, NNY, DWR, ERA, QRE, EVR, SRD, AWA, SFA, QTI, SHT, THM, AQI, AHI, SWT, ATV, TTI, SMI, QYV, NMY, SWM, QNY, TWT, QWT, TQY, NRE, QTS, QHQ, DVR, AAA, TYA, EKH, ALH, AWT, ATH, SRE, TFH, QMH, ERV, ERS, QHY, ALQ, QSY, ERH, TRE, TSS, QAH, AAH, SWA, TWS, QWS, AYS, AFV, QNQ, ANY, ALT, SMY, TWV, TTY, AMY, QMY, ERY, TWH, TSY, TAY, TYV, QAY, AAY, QYQ, TYQ, SYH, AQV, EER, THQ, SMT.

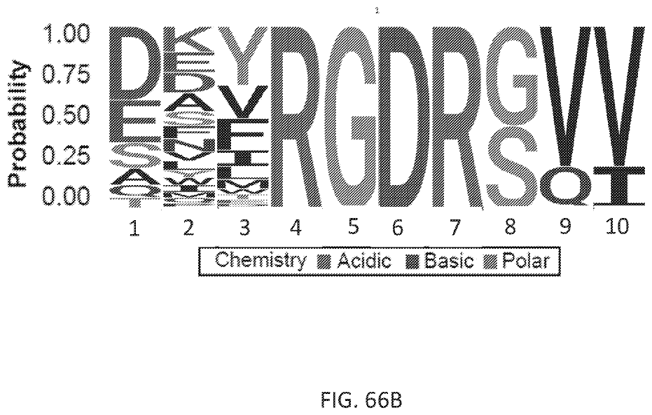

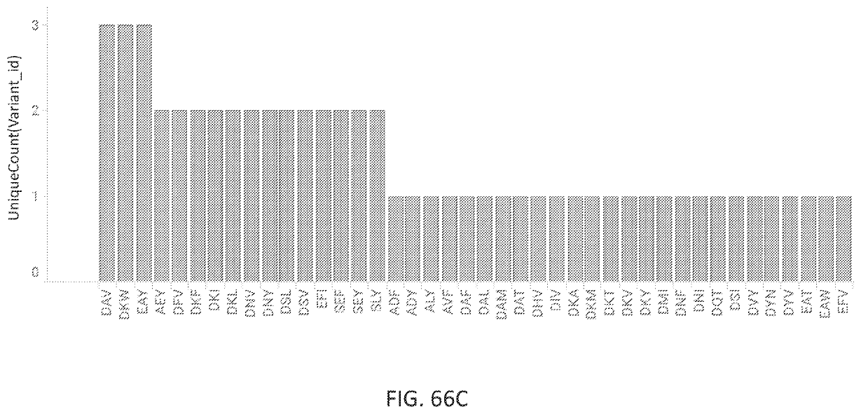

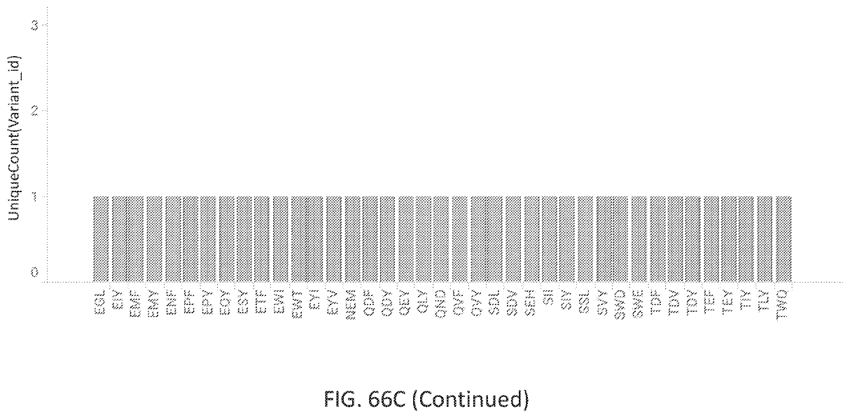

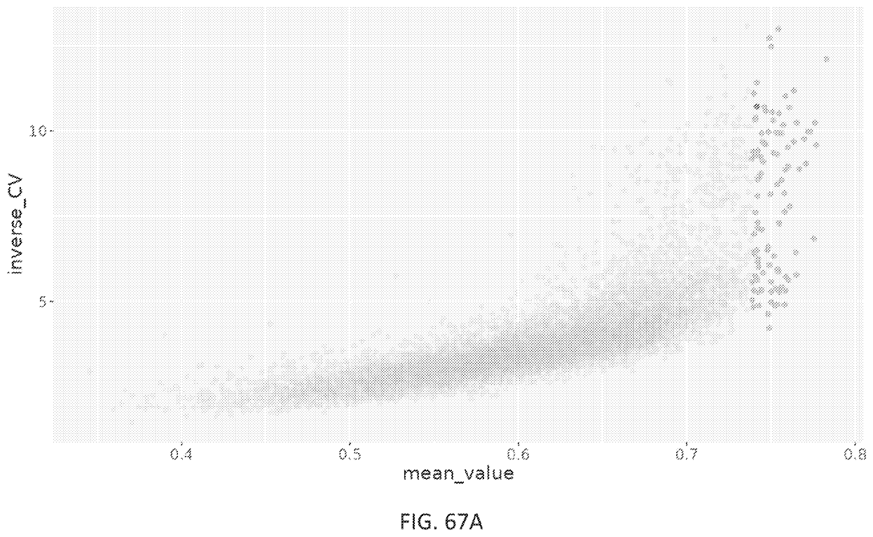

Analysis of capsids in Group 1 is shown in FIGS. 66A-66C. Group 1 included capsids comprising targeting peptides having a quad selected from: RGQI, RSVV, and RGVV. FIG. 66A shows enrichment scores averaged across all muscle tissue for each of the capsids in the AAV^{-Lib2} library having a quad selected from: RGQI, RSVV, and RGVV. The top 100 capsids are identified by the larger of the two dots on the plot. A motif analysis was performed to determine if there was a pattern of motifs enriched in the top 100 capsids in FIG. 66A. FIG. 66B shows the motif plot that identifies the enriched motifs in the top 100. Additional analysis of the triplets for the top 100 capsids was provided in FIG. 66C. For example, FIG. 66C shows the unique variant count of the triplet sequences present in the top 100 capsids in FIG. 66A. Interestingly, this data shows that for Group 1 the X₁ position favors amino acid residues D or E and the X₃ position favors amino acid residues Y, V, or F.

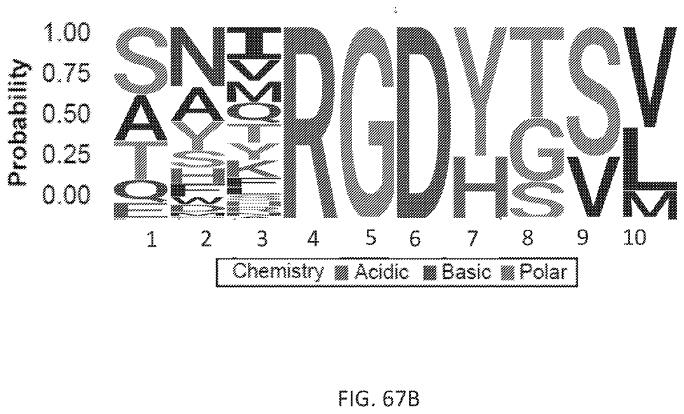

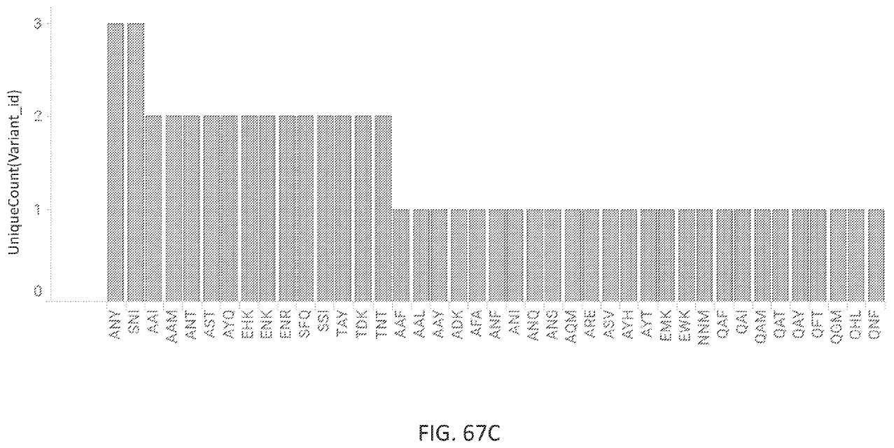

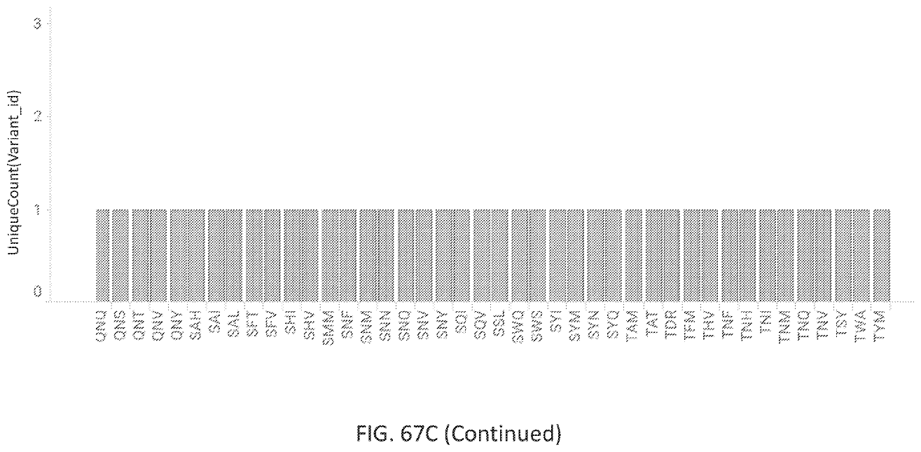

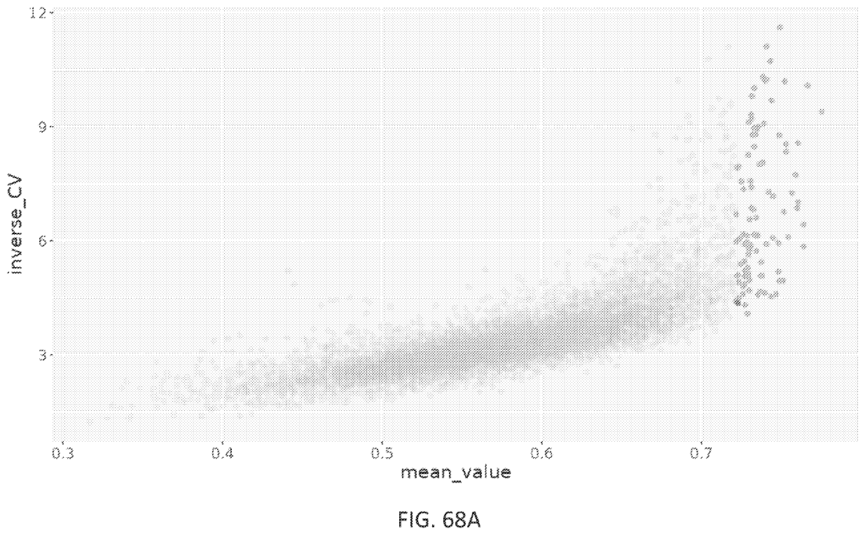

Analysis of capsids in Group 2 is shown in FIGS. 67A-67C. Group 2 included capsids comprising targeting peptides having a quad selected from: HGVL, YSSV, YSTM, and YTSV. FIG. 67A shows enrichment scores averaged across all muscle tissue for each of the capsids in the AAV^{-Lib2} library having a quad selected from: HGVL, YSSV, YSTM, and YTSV. The top 100 capsids are identified by the larger of the two dots on the plot. A motif analysis was performed to determine if there was a pattern of motifs enriched in the top 100 capsids in FIG. 67A. FIG. 67B shows the motif plot that identifies the enriched motifs in the top 100. Additional analysis of the triplets for the top 100 capsids was provided in FIG. 67C. In particular, FIG. 67C shows the unique variant count of the triplet sequences present in the top 100 capsids in FIG. 67A. Interestingly, this data shows that for Group 2 the X₁ position favors amino acid residues S, A, or T and the X₃ position favors amino acid residues N, A, or Y.

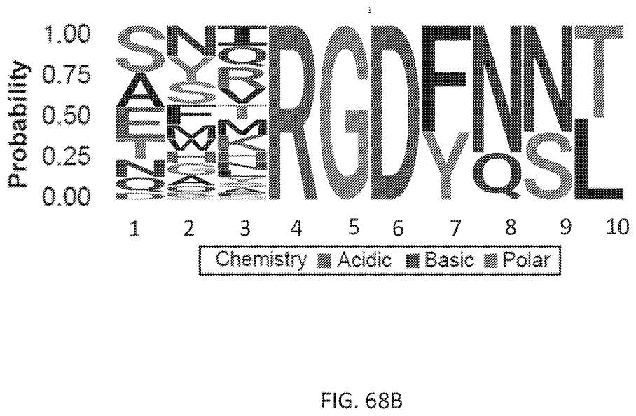

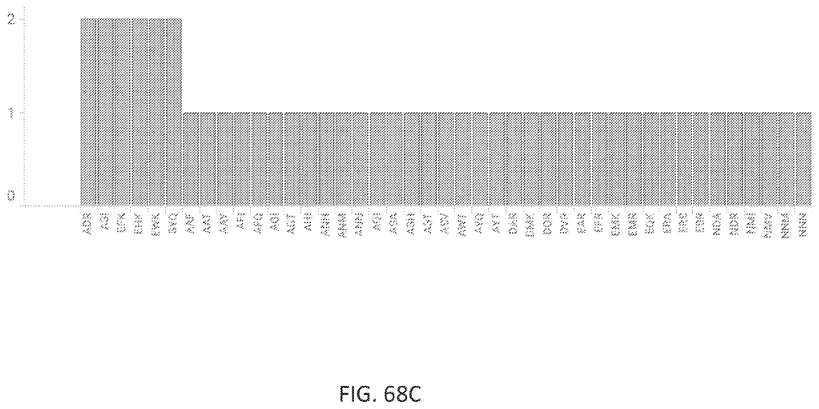

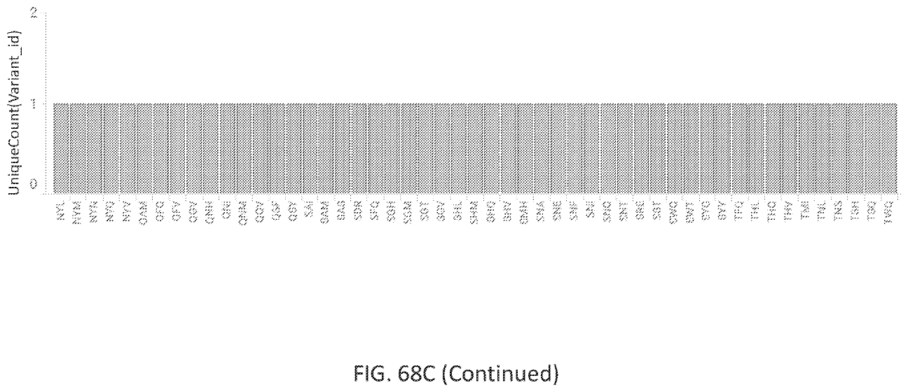

Analysis of capsids in Group 3 is shown in FIGS. 68A-68C. Group 3 included capsids comprising targeting peptides having a quad selected from: HGVL, YSSV. YSTM, and YTSV. FIG. 68A shows enrichment scores averaged across all muscle tissue for each of the capsids in the AAV^{-Lib2} library having a quad selected from: FNNT, FNNL. FQNT, and YNSL. The top 100 capsids are identified by the larger of the two dots on the plot. A motif analysis was performed to determine if there was a pattern of motifs enriched in the top 100 capsids in FIG. 68A. FIG. 68B shows the motif plot that identifies the enriched motifs in the top 100. Additional analysis of the triplets for the top 100 capsids was provided in FIG. 68C. In particular, FIG. 68C shows the unique variant count of the triplet sequences present in the top 100 capsids in FIG. 68A. Interestingly, this data shows that for Group 3 the X₁ position favors amino acid residues S, A, or E and the X₃ position favors amino acid residues N, Y, or S.

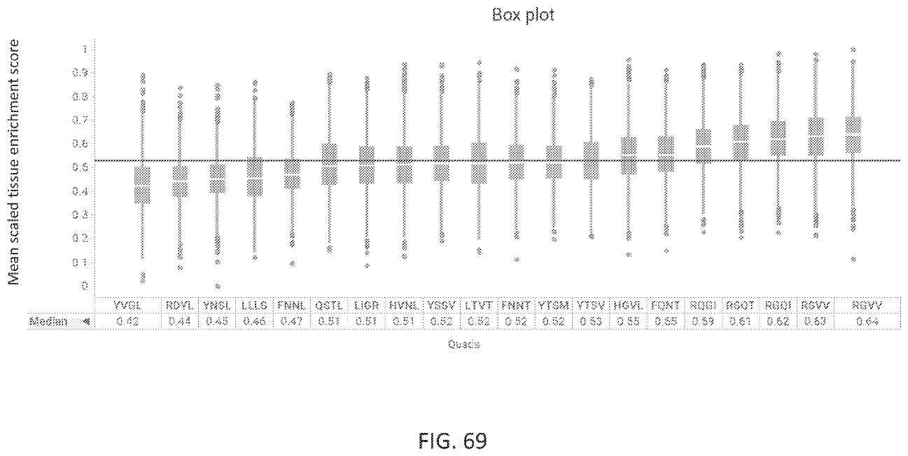

Tissue enrichment in liver tissue was also assessed for the AAV^{-Lib2} library. In particular, FIG. 69 shows the amount of vector RNA in liver tissue for the sum of all the capsids in the AAV^{-Lib2} library having the indicated quad sequences. This analysis revealed that targeting peptides having RGVV, RSVV, RGQI, RSQT, and RQGI were the most liver tropic quads.

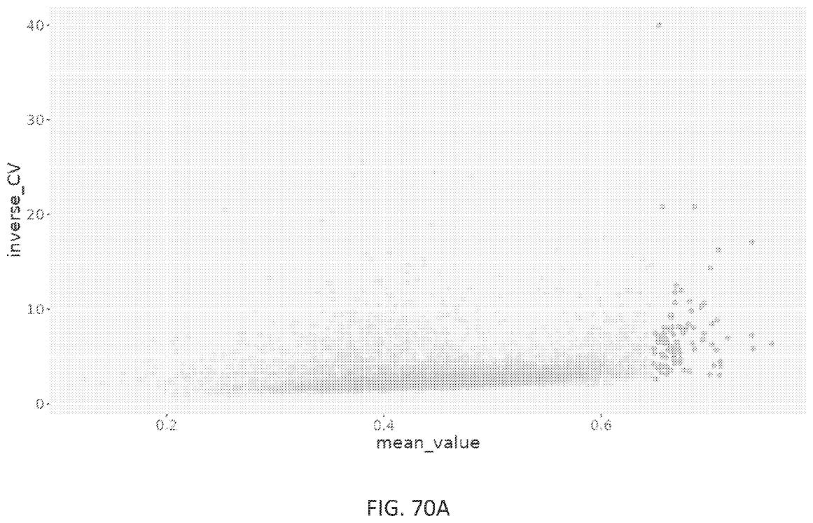

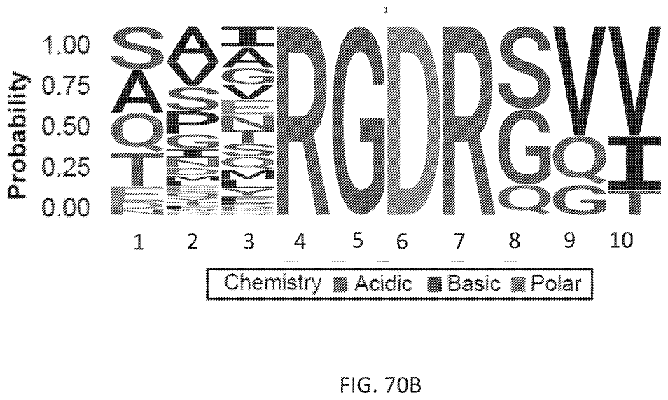

Additional analysis of capsids with highest liver enrichment is shown in FIGS. 70A-70B. As shown in FIG. 70A enrichment scores averaged across all liver tissue for each of the capsids in the AAV^{-Lib2} library enabled identification of the top 100 liver tropic capsids. The top 100 capsids are identified by the larger of the two dots on the plot (See FIG. 70A). A motif analysis was performed on the top 100 liver tropic capsids to determine if there was a pattern of motifs enriched in the top 100 capsids. FIG. 70B shows the enriched motifs in the top 100 capsids. Interestingly, this data shows that the triplets enriched in the muscle are different from the triplets enriched in the liver.

### 8.27.1. Top 10 Variants

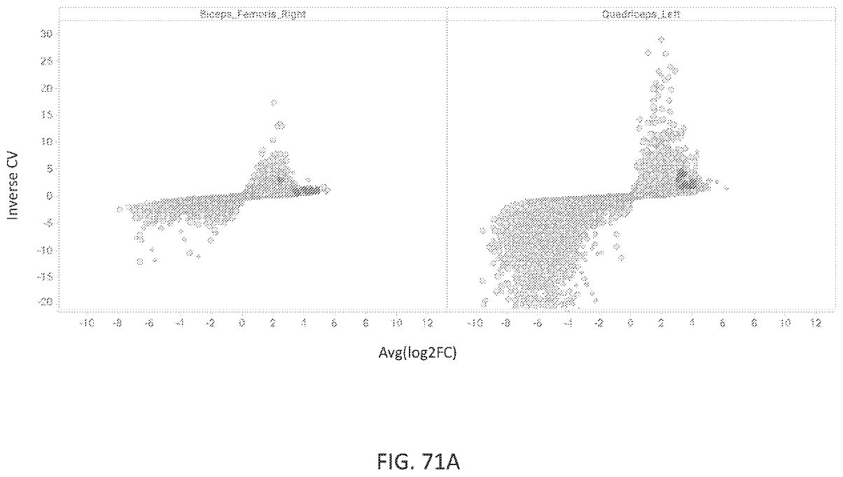

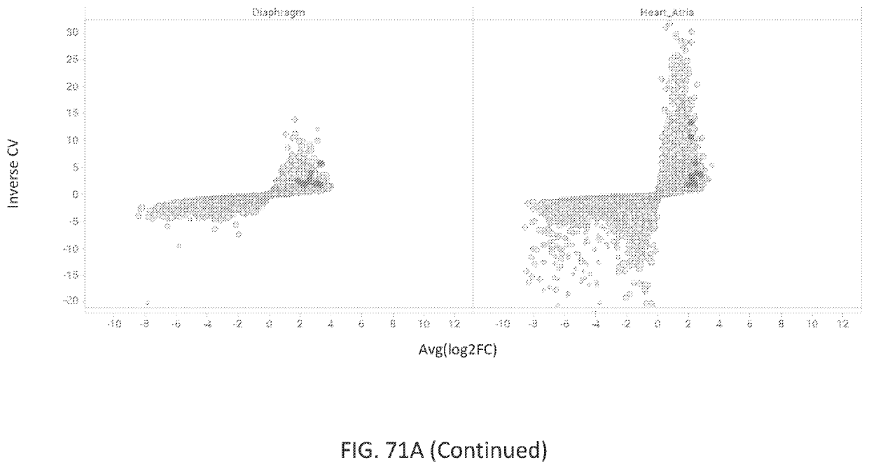

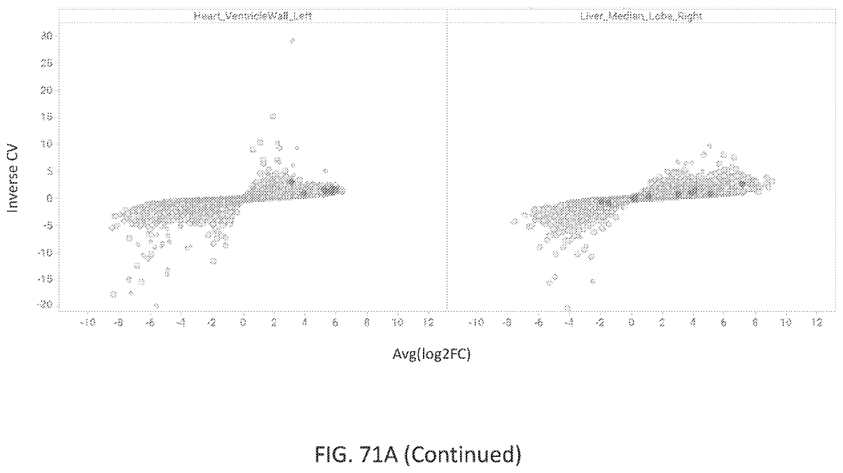

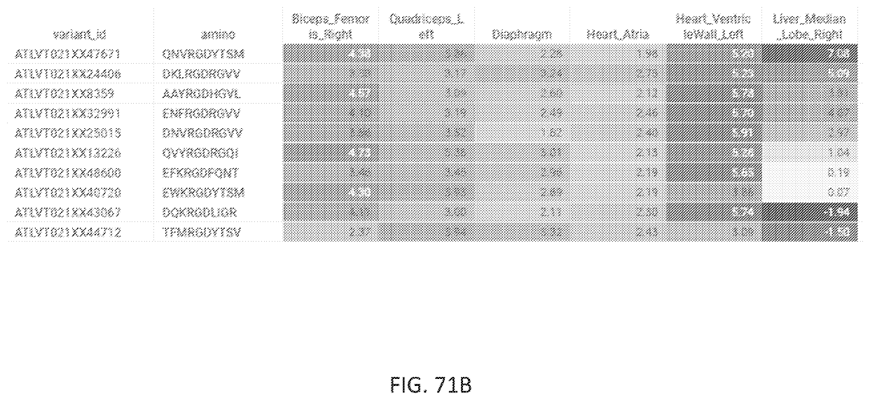

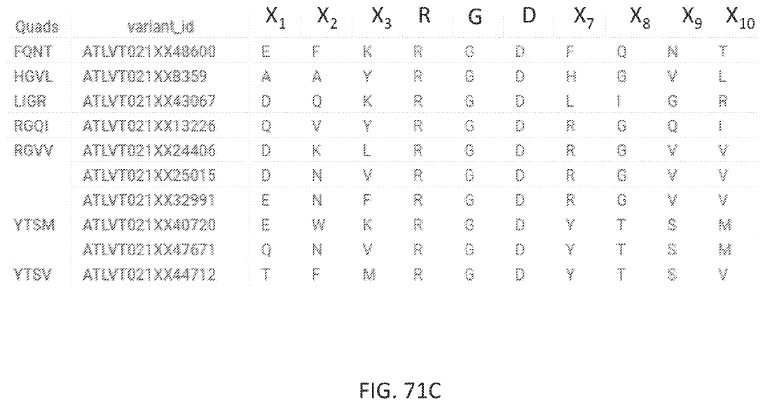

From the AAV^{-Lib2} data, the top 10 muscle tropic capsids were identified and further analyzed (see FIGS. 71A-71C, 72A-72B, 73A-73B, and 74A-74B).

FIG. 71A shows enrichment score plots (inverse CV versus AverageMN_FC) for biceps femoris, quadriceps, diaphragm, heart-atria, heart-ventricle, and liver for each of the capsids in the AAV^{-Lib2} library. The top 10 performing capsids are identified by the darker shaded circles. Tissue enrichment scores for each capsid in the tissues from FIG. 71A are shown in FIG. 71B and FIG. 71C. The top 10 targeting peptides included triplets selected from: EFK, AAY, DQK. QVY, DKL, DNV. ENF, EWK, QNV, and TFM. The top 10 muscle variants ranked consistently high in all muscle tissues and moderate in liver.

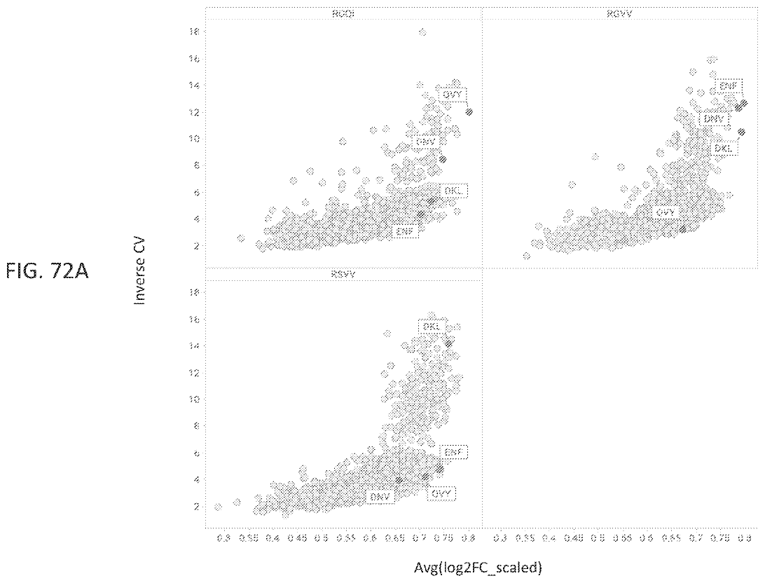

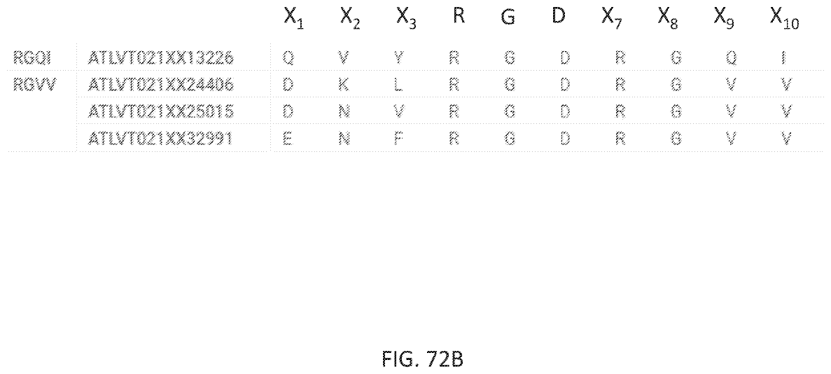

FIG. 72A-72B looked at the how the triplets from the top 10 targeting peptides performed when combined with various quads. In particular, FIG. 72A shows the enrichment scores for capsids having quads selected from RGQI, RGVV, and RSVV and targeting peptides having the triplets identified in the top 10 targeting peptides. FIG. 72B provides the amino acid residues for the relevant targeting peptides in FIG. 72A.

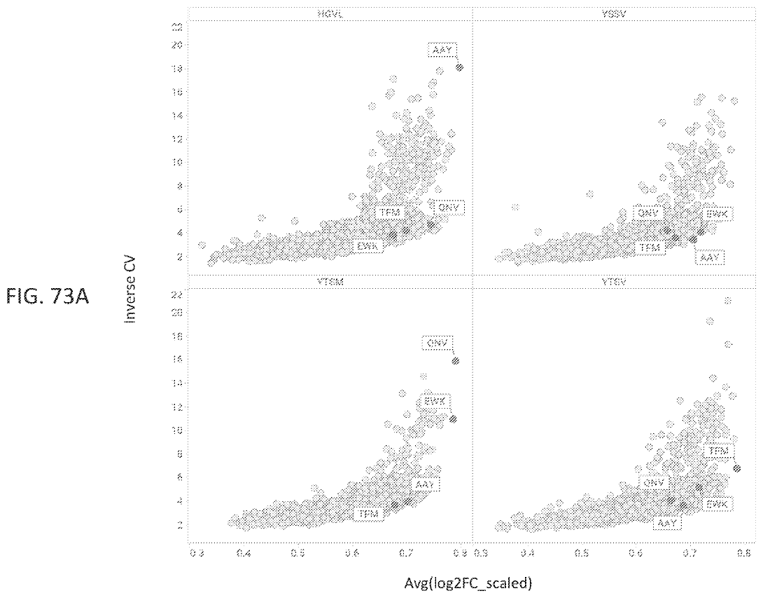

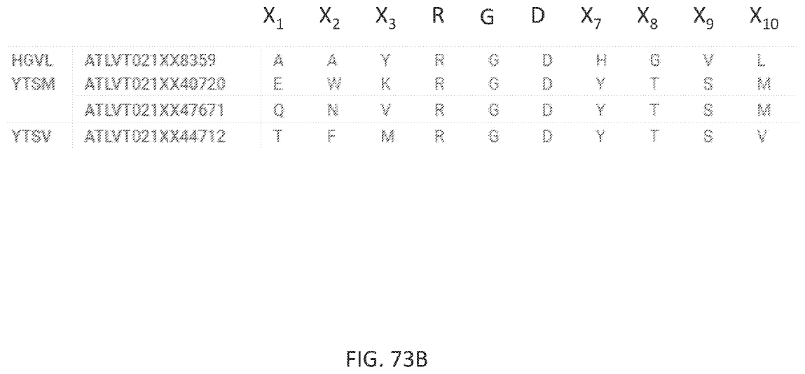

FIG. 73A-73B looked at the how the triplets from the top 10 targeting peptides performed when combined with other quads. FIG. 73A shows the enrichment scores for capsids having quads selected from HGVL, YTSM. YSSV, and YTSV and targeting peptides having the triplets identified in the top 10 targeting peptides. FIG. 73B provides the amino acid residues for the relevant targeting peptides in FIG. 73A.

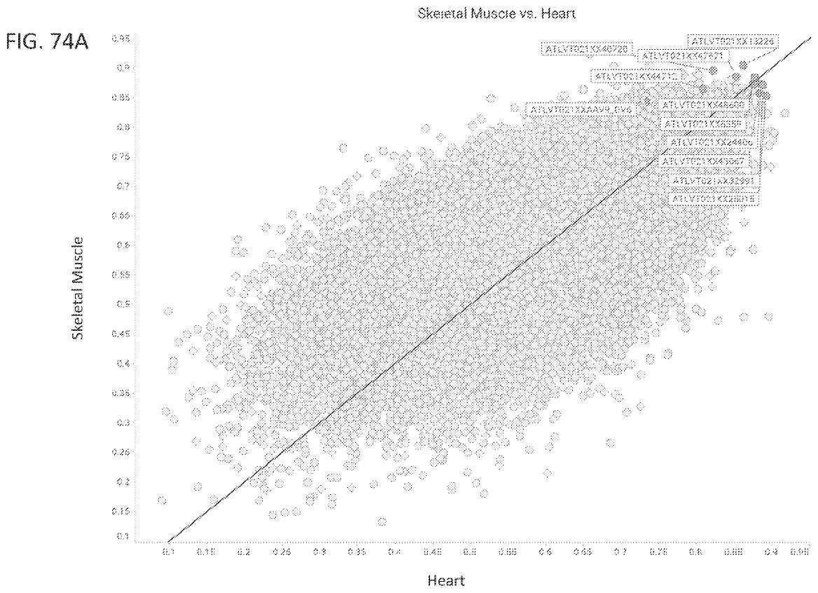

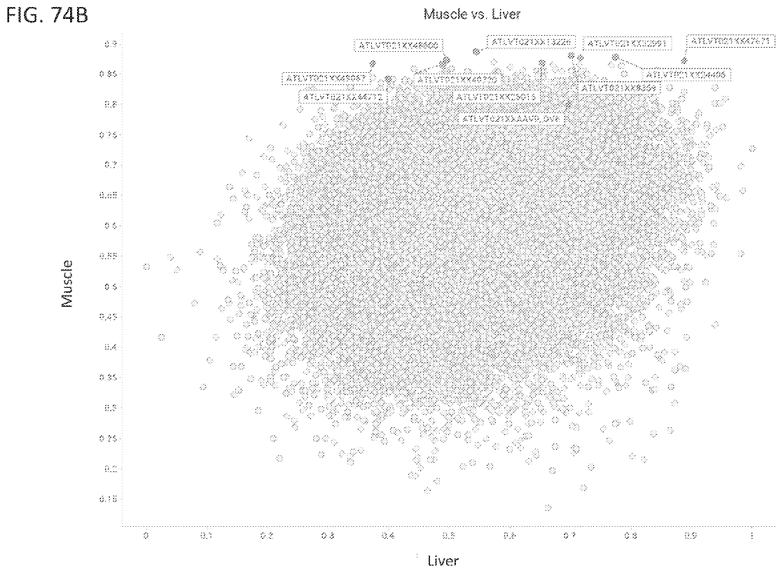

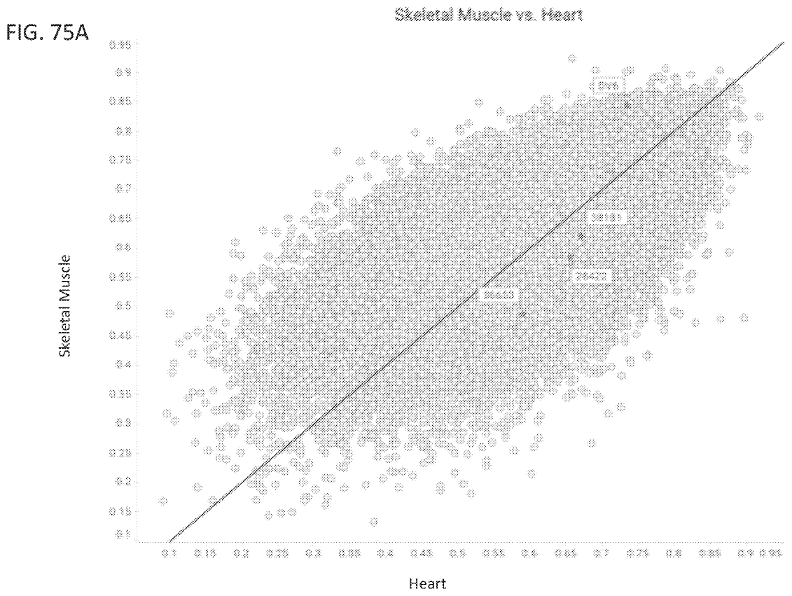

FIG. 74A-74B looked at how the top 10 targeting peptides performed compared to capsids: targeting peptide combinations identified in other studies (sec. Example 25). As shown in FIG. 74A, the top 10 targeting peptides performed similarly to previously identified targeting peptides when comparing enrichment in skeletal muscle versus heart. FIG. 74B shows how the top 10 targeting peptides performed when comparing enrichment in muscle versus liver. Overall, this data showed that the current study could successfully identify targeting peptides with muscle tropism and that these new targeting peptides performed as well or better than targeting peptides identified elsewhere herein.

### 8.27.2. Heart-Specific Targeting Peptides

The AAV^{-Lib2} library data was also analyzed to assess whether certain capsids had increased tropism for a particular subset of muscle tissue, for example, increased tropism in the heart over skeletal muscle or increased tropism in skeletal muscle over the heart.

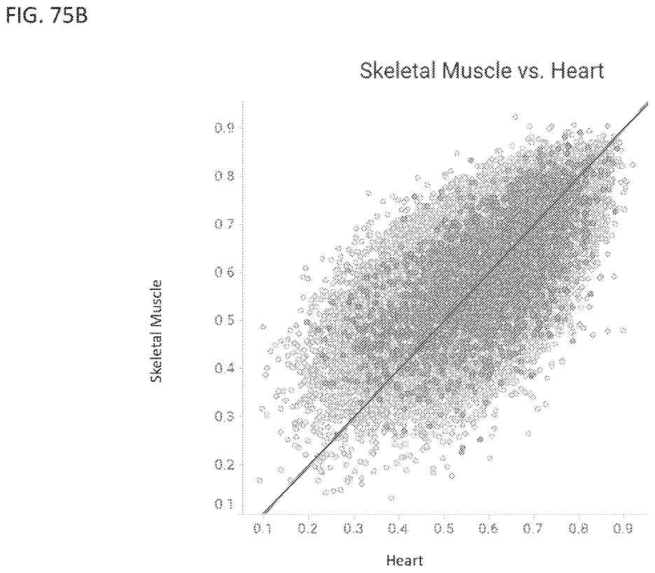

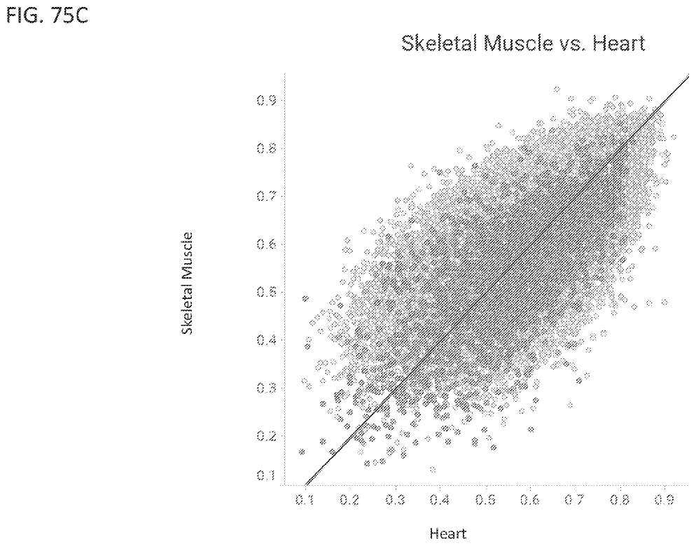

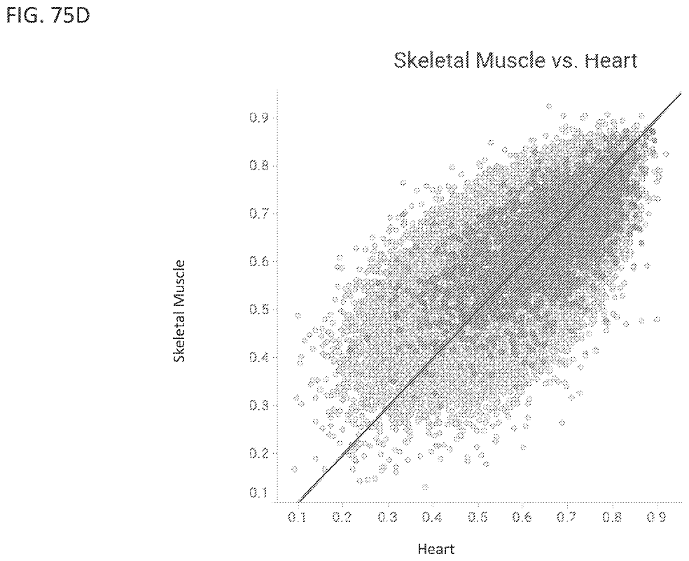

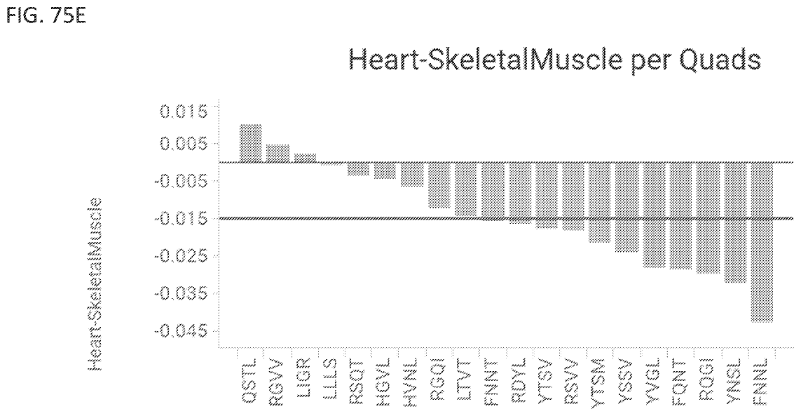

As shown in FIG. 75A, the present study included capsids having a variety of tissue enrichment profiles as some capsids favored skeletal muscle over heart muscle and vice versa. In particular, analysis of the data in FIG. 75A revealed that targeting peptides having quads selected from: QSTL (36653), LIGR (28422), and RGVV were the most “heart-specific” quads.

FIG. 75B shows enrichment scores in skeletal muscle versus heart for each of the capsids in the AAV^{-Lib2} library having the indicated quad (“LIGR”).

FIG. 75C shows enrichment scores in skeletal muscle versus heart for each of the capsids in the AAV^{-Lib2} library having the indicated quad (“QSTL”).

FIG. 75D shows enrichment scores in skeletal muscle versus heart for each of the capsids in the AAV^{-Lib2} library having the indicated quad (“RGVV”).

FIG. 75E shows enrichment scores for heart over skeletal muscle for each of the capsids in the AAV^{-Lib2} library having the indicated quads.

### 8.27.3. Conclusion

Overall, this data shows that a subset of the modified AAV capsid comprising various targeting peptides tested in NHP have increased muscle tropism. Further analysis revealed that a subset of the targeting peptides were enriched in skeletal muscle over heart muscle and a second subset of the targeting peptides were enriched in heart muscle over skeletal muscle.

### 9. SEQUENCES

Many of the nucleotide sequences provided below are obtained from double stranded vectors. Thus, one of skill in the art would appreciate that, unless the references throughout the specification and claims to nucleotide sequences provided herein also include references to the complementary sequences unless the context dictates otherwise.

| | SEQUENCE (X or X″ can be any of the standard amino acids; |
|---|---|
| | for Anc library sequences (Anc80; Anc81; Anc82; Anc83; |
| | Anc84; Anc94; Anc110; Anc113; Anc126; and Anc127), |
| SEQ ID | X can be any one of the amino acids listed |
| NO | below for each toggle site) |
| SEQ ID | RGDLLLS |
| NO: 1 | |
| SEQ ID | AQTLAWPFKAQ |
| NO: 2 | |
| SEQ ID | AQSWSKPFLAQ |
| NO: 3 | |
| SEQ ID | DGTLAVPFKAQ |
| NO: 4 | |
| SEQ ID | ESTLAVPFKAQ |
| NO: 5 | |
| SEQ ID | ESTLAVPFKAQ |
| NO: 6 | |
| SEQ ID | GGTLAVPFKAQ |
| NO: 7 | |
| SEQ ID | AQTLATPFKAQ |
| NO: 8 | |
| SEQ ID | ATTLATPFKAQ |
| NO: 9 | |
| SEQ ID | DGTLATPFKAQ |
| NO: 10 | |
| SEQ ID | GGTLATPFKAQ |
| NO: 11 | |
| SEQ ID | SGSLAWPFKAQ |
| NO: 12 | |
| SEQ ID | AQTLAQPFKAQ |
| NO: 13 | |
| SEQ ID | AQTLQQPFKAQ |
| NO: 14 | |
| SEQ ID | AQTLSNPFKAQ |
| NO: 15 | |
| SEQ ID | AQTLAVPFSNP |
| NO: 16 | |
| SEQ ID | QGTLAVPFKAQ |
| NO: 17 | |
| SEQ ID | NQTLAVPFKAQ |
| NO: 18 | |
| SEQ ID | EGSLAVPFKAQ |
| NO: 19 | |
| SEQ ID | SGNLAVPFKAQ |
| NO: 20 | |
| SEQ ID | EGTLAVPFKAQ |
| NO: 21 | |
| SEQ ID | DSTLAVPFKAQ |
| NO: 22 | |
| SEQ ID | AVTLAVPFKAQ |
| NO: 23 | |
| SEQ ID | AQTLSTPFKAQ |
| NO: 24 | |
| SEQ ID | AQTLPQPFKAQ |
| NO: 25 | |
| SEQ ID | AQTLSQPFKAQ |
| NO: 26 | |
| SEQ ID | AQTLQLPFKAQ |
| NO: 27 | |
| SEQ ID | AQTLTMPFKAQ |
| NO: 28 | |
| SEQ ID | AQTLTTPFKAQ |
| NO: 29 | |
| SEQ ID | AQYTLSQGWAQ |
| NO: 30 | |
| SEQ ID | AQMNATKNVAQ |
| NO: 31 | |
| SEQ ID | AQVSGGHHSAQ |
| NO: 32 | |
| SEQ ID | AQTLPQPFKAQ |
| NO: 33 | |
| SEQ ID | AQTLATPFKAQ |
| NO: 34 | |
| SEQ ID | AQTLTMPFKAQ |
| NO: 35 | |
| SEQ ID | AQTLTAPFKAQ |
| NO: 36 | |
| SEQ ID | AQTLSKPFKAQ |
| NO: 37 | |
| SEQ ID | QAVRTSL |
| NO: 38 | |
| SEQ ID | YTLSQGW |
| NO: 39 | |
| SEQ ID | LAKERLS |
| NO: 40 | |
| SEQ ID | LAKERLS |
| NO: 41 | |
| SEQ ID | SVSKPFL |
| NO: 42 | |
| SEQ ID | FTLTTPK |
| NO: 43 | |
| SEQ ID | MNSTKNV |
| NO: 44 | |
| SEQ ID | VSGGHHS |
| NO: 45 | |
| SEQ ID | SAQTLAVPFKAQAQ |
| NO: 46 | |
| SEQ ID | SXXXLAVPFKAQAQ |
| NO: 47 | |
| SEQ ID | SAQXXXVPFKAQAQ |
| NO: 48 | |
| SEQ ID | SAQTLXXXFKAQAQ |
| NO: 49 | |
| SEQ ID | SAQTLAVXXXAQAQ |
| NO: 50 | |
| SEQ ID | SAQTLAVPFXXXAQ |
| NO: 51 | |
| RGD | RGDX₁X₂X₃X₄ |
| consensus | |
| SEQ ID | TLAVPFK |
| NO: 53 | |
| SEQ ID | MAADGYLPDWLEDNLSEGIREWWDLKPGAPKPKANQQKQDDGRGLVLPGY |
| NO: 54 | KYLGPFNGLDKGEPVNAADAAALEHDKAYDQQLKAGDNPYLRYNHADAEF |
| (AAV1 | QERLQEDTSFGGNLGRAVFQAKKRVLEPLGLVEEGAKTAPGKKRPVEQSP |
| (AAD27757)) | QEPDSSSGIGKTGQQPAKKRLNFGQTGDSESVPDPQPLGEPPATPAAVGP |
| | TTMASGGGAPMADNNEGADGVGNASGNWHCDSTWLGDRVITTSTRTWALP |
| | TYNNHLYKQISSASTGASNDNHYFGYSTPWGYFDFNRFHCHFSPRDWQRL |
| | INNNWGFRPKRLNFKLFNIQVKEVTTNDGVTTIANNLTSTVQVFSDSEYQ |
| | LPYVLGSAHQGCLPPFPADVFMIPQYGYLTLNNGSQAVGRSSFYCLEYFP |
| | SQMLRTGNNFTFSYTFEEVPFHSSYAHSQSLDRLMNPLIDQYLYYLNRTQ |
| | NQSGSAQNKDLLFSRGSPAGMSVQPKNWLPGPCYRQQRVSKTKTDNNNSN |
| | FTWTGASKYNLNGRESIINPGTAMASHKDDEDKFFPMSGVMIFGKESAGA |
| | SNTALDNVMITDEEEIKATNPVATERFGTVAVNFQSSSTDPATGDVHAMG |
| | ALPGMVWQDRDVYLQGPIWAKIPHTDGHFHPSPLMGGFGLKNPPPQILIK |
| | NTPVPANPPAEFSATKFASFITQYSTGQVSVEIEWELQKENSKRWNPEVQ |
| | YTSNYAKSANVDFTVDNNGLYTEPRPIGTRYLTRPL |
| SEQ ID | MAADGYLPDWLEDTLSEGIRQWWKLKPGPPPPKPAERHKDDSRGLVLPGY |
| NO: 55 | KYLGPFNGLDKGEPVNEADAAALEHDKAYDRQLDSGDNPYLKYNHADAEF |
| (AAV2 | QERLKEDTSFGGNLGRAVFQAKKRVLEPLGLVEEPVKTAPGKKRPVEHSP |
| (AAC03780)) | VEPDSSSGTGKAGQQPARKRLNFGQTGDADSVPDPQPLGQPPAAPSGLGT |
| | NTMATGSGAPMADNNEGADGVGNSSGNWHCDSTWMGDRVITTSTRTWALP |
| | TYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQL |
| | PYVLGSAHQGCLPPFPADVFMVPQYGYLTLNNGSQAVGRSSFYCLEYFPS |
| | QMLRTGNNFTFSYTFEDVPFHSSYAHSQSLDRLMNPLIDQYLYYLSRTNT |
| | PSGTTTQSRLQFSQAGASDIRDQSRNWLPGPCYRQQRVSKTSADNNNSEY |
| | SWTGATKYHLNGRDSLVNPGPAMASHKDDEEKFFPQSGVLIFGKQGSEKT |
| | NVDIEKVMITDEEEIRTTNPVATEQYGSVSTNLQRGNRQAATADVNTQGV |
| | LPGMVWQDRDVYLQGPIWAKIPHTDGHFHPSPLMGGFGLKHPPPQILIKN |
| | TPVPANPSTTFSAAKFASFITQYSTGQVSVEIEWELQKENSKRWNPEIQY |
| | TSNYNKSVNVDFTVDINGVYSEPRPIGTRYLTRNL |
| SEQ ID | MAADGYLPDWLEDNLSEGIREWWALKPGVPQPKANQQHQDNRRGLVLPGY |
| NO: 56 | KYLGPGNGLDKGEPVNEADAAALEHDKAYDQQLKAGDNPYLKYNHADAEF |
| (AAV3 | QERLQEDTSFGGNLGRAVFQAKKRILEPLGLVEEAAKTAPGKKGAVDQSP |
| (AAC55049)) | QEPDSSSGVGKSGKQPARKRLNFGQTGDSESVPDPQPLGEPPAAPTSLGS |
| | NTMASGGGAPMADNNEGADGVGNSSGNWHCDSQWLGDRVITTSTRTWALP |
| | TYNNHLYKQISSQSGASNDNHYFGYSTPWGYFDFNRFHCHFSPRDWQRLI |
| | NNNWGFRPKKLSFKLFNIQVRGVTQNDGTTTIANNLTSTVQVFTDSEYQL |
| | PYVLGSAHQGCLPPFPADVFMVPQYGYLTLNNGSQAVGRSSFYCLEYFPS |
| | QMLRTGNNFQFSYTFEDVPFHSSYAHSQSLDRLMNPLIDQYLYYLNRTQG |
| | TTSGTTNQSRLLFSQAGPQSMSLQARNWLPGPCYRQQRLSKTANDNNNSN |
| | FPWTAASKYHLNGRDSLVNPGPAMASHKDDEEKFFPMHGNLIFGKEGTTA |
| | SNAELDNVMITDEEEIRTTNPVATEQYGTVANNLQSSNTAPTTGTVNHQG |
| | ALPGMVWQDRDVYLQGPIWAKIPHTDGHFHPSPLMGGFGLKHPPPQIMIK |
| | NTPVPANPPTTFSPAKFASFITQYSTGQVSVEIEWELQKENSKRWNPEIQ |
| | YTSNYNKSVNVDFTVDTNGVYSEPRPIGTRYLTRNL |
| SEQ ID | MSFVDHPPDWLEEVGEGLREFLGLEAGPPKPKPNQQHQDQARGLVLPGYN |
| NO: 57 | YLGPGNGLDRGEPVNRADEVAREHDISYNEQLEAGDNPYLKYNHADAEFQ |
| (AAV5 | EKLADDTSFGGNLGKAVFQAKKRVLEPFGLVEEGAKTAPTGKRIDDHFPK |
| (AAD13756)) | RKKARTEEDSKPSTSSDAEAGPSGSQQLQIPAQPASSLGADTMSAGGGGP |
| | LGDNNQGADGVGNASGDWHCDSTWMGDRVVTKSTRTWVLPSYNNHQYREI |
| | KSGSVDGSNANAYFGYSTPWGYFDFNRFHSHWSPRDWQRLINNYWGFRPR |
| | SLRVKIFNIQVKEVTVQDSTTTIANNLTSTVQVFTDDDYQLPYVVGNGTE |
| | GCLPAFPPQVFTLPQYGYATLNRDNTENPTERSSFFCLEYFPSKMLRTGN |
| | NFEFTYNFEEVPFHSSFAPSQNLFKLANPLVDQYLYRFVSTNNTGGVQFN |
| | KNLAGRYANTYKNWFPGPMGRTQGWNLGSGVNRASVSAFATTNRMELEGA |
| | SYQVPPQPNGMINNLQGSNTYALENTMIFNSQPANPGTTATYLEGNMLIT |
| | SESETQPVNRVAYNVGGQMATNNQSSTTAPATGTYNLQEIVPGSVWMERD |
| | VYLQGPIWAKIPETGAHFHPSPAMGGFGLKHPPPMMLIKNTPVPGNITSF |
| | SDVPVSSFITQYSTGQVTVEMEWELKKENSKRWNPEIQYTNNYNDPQFVD |
| | FAPDSTGEYRTTRPIGTRYLTRPL |
| SEQ ID | MAADGYLPDWLEDNLSEGIREWWDLKPGAPKPKANQQKQDDGRGLVLPGY |
| NO: 58 | KYLGPFNGLDKGEPVNAADAAALEHDKAYDQQLKAGDNPYLRYNHADAEF |
| (AAV6 | QERLQEDTSFGGNLGRAVFQAKKRVLEPFGLVEEGAKTAPGKKRPVEQSP |
| (AAB95450)) | QEPDSSSGIGKTGQQPAKKRLNFGQTGDSESVPDPQPLGEPPATPAAVGP |
| | TTMASGGGAPMADNNEGADGVGNASGNWHCDSTWLGDRVITTSTRTWALP |
| | TYNNHLYKQISSASTGASNDNHYFGYSTPWGYFDFNRFHCHFSPRDWQRL |
| | INNNWGFRPKRLNFKLFNIQVKEVTTNDGVTTIANNLTSTVQVFSDSEYQ |
| | LPYVLGSAHQGCLPPFPADVFMIPQYGYLTLNNGSQAVGRSSFYCLEYFP |
| | SQMLRTGNNFTFSYTFEDVPFHSSYAHSQSLDRLMNPLIDQYLYYLNRTQ |
| | NQSGSAQNKDLLFSRGSPAGMSVQPKNWLPGPCYRQQRVSKTKTDNNNSN |
| | FTWTGASKYNLNGRESIINPGTAMASHKDDKDKFFPMSGVMIFGKESAGA |
| | SNTALDNVMITDEEEIKATNPVATERFGTVAVNLQSSSTDPATGDVHVMG |
| | ALPGMVWQDRDVYLQGPIWAKIPHTDGHFHPSPLMGGFGLKHPPPQILIK |
| | NTPVPANPPAEFSATKFASFITQYSTGQVSVEIEWELQKENSKRWNPEVQ |
| | YTSNYAKSANVDFTVDNNGLYTEPRPIGTRYLTRPL |
| SEQ ID | MAADGYLPDWLEDNLSEGIREWWDLKPGAPKPKANQQKQDNGRGLVLPGY |
| NO: 59 | KYLGPFNGLDKGEPVNAADAAALEHDKAYDQQLKAGDNPYLRYNHADAEF |
| (AAV7 | QERLQEDTSFGGNLGRAVFQAKKRVLEPLGLVEEGAKTAPAKKRPVEPSP |
| (AF513851_2)) | QRSPDSSTGIGKKGQQPARKRLNFGQTGDSESVPDPQPLGEPPAAPSSVG |
| | SGTVAAGGGAPMADNNEGADGVGNASGNWHCDSTWLGDRVITTSTRTWAL |
| | PTYNNHLYKQISSETAGSTNDNTYFGYSTPWGYFDFNRFHCHFSPRDWQR |
| | LINNNWGFRPKKLRFKLFNIQVKEVTTNDGVTTIANNLTSTIQVESDSEY |
| | QLPYVLGSAHQGCLPPFPADVFMIPQYGYLTLNNGSQSVGRSSFYCLEYF |
| | PSQMLRTGNNFEFSYSFEDVPFHSSYAHSQSLDRLMNPLIDQYLYYLART |
| | QSNPGGTAGNRELQFYQGGPSTMAEQAKNWLPGPCFRQQRVSKTLDQNNN |
| | SNFAWTGATKYHLNGRNSLVNPGVAMATHKDDEDRFFPSSGVLIFGKTGA |
| | TNKTTLENVLMTNEEEIRPTNPVATEEYGIVSSNLQAANTAAQTQVVNNQ |
| | GALPGMVWQNRDVYLQGPIWAKIPHTDGNFHPSPLMGGFGLKHPPPQILI |
| | KNTPVPANPPEVFTPAKFASFITQYSTGQVSVEIEWELQKENSKRWNPEI |
| | QYTSNFEKQTGVDFAVDSQGVYSEPRPIGTRYLTRNL |
| SEQ ID | MAADGYLPDWLEDNLSEGIREWWALKPGAPKPKANQQKQDDGRGLVLPGY |
| NO: 60 | KYLGPFNGLDKGEPVNAADAAALEHDKAYDQQLQAGDNPYLRYNHADAEF |
| (AAV8 | QERLQEDTSFGGNLGRAVFQAKKRVLEPLGLVEEGAKTAPGKKRPVEPSP |
| (AF513852_2)) | QRSPDSSTGIGKKGQQPARKRLNFGQTGDSESVPDPQPLGEPPAAPSGVG |
| | PNTMAAGGGAPMADNNEGADGVGSSSGNWHCDSTWLGDRVITTSTRTWAL |
| | PTYNNHLYKQISNGTSGGATNDNTYFGYSTPWGYFDFNRFHCHFSPRDWQ |
| | RLINNNWGFRPKRLSFKLFNIQVKEVTQNEGTKTIANNLTSTIQVFTDSE |
| | YQLPYVLGSAHQGCLPPFPADVFMIPQYGYLTLNNGSQAVGRSSFYCLEY |
| | FPSQMLRTGNNFQFTYTFEDVPFHSSYAHSQSLDRLMNPLIDQYLYYLSR |
| | TQTTGGTANTQTLGFSQGGPNTMANQAKNWLPGPCYRQQRVSTTTGQNNN |
| | SNFAWTAGTKYHLNGRNSLANPGIAMATHKDDEERFFPSNGILIFGKQNA |
| | ARDNADYSDVMLTSEEEIKTTNPVATEEYGIVADNLQQQNTAPQIGTVNS |
| | QGALPGMVWQNRDVYLQGPIWAKIPHTDGNFHPSPLMGGFGLKHPPPQIL |
| | IKNTPVPADPPTTFNQSKLNSFITQYSTGQVSVEIEWELQKENSKRWNPE |
| | IQYTSNYYKSTSVDFAVNTEGVYSEPRPIGTRYLTRNL |
| SEQ ID | MAADGYLPDWLEDNLSEGIREWWALKPGAPQPKANQQHQDNARGLVLPGY |
| NO: 61 | KYLGPGNGLDKGEPVNAADAAALEHDKAYDQQLKAGDNPYLKYNHADAEF |
| (AAV9 | QERLKEDTSFGGNLGRAVFQAKKRLLEPLGLVEEAAKTAPGKKRPVEQSP |
| (AAS99264)) | QEPDSSAGIGKSGAQPAKKRLNFGQTGDTESVPDPQPIGEPPAAPSGVGS |
| | LTMASGGGAPVADNNEGADGVGSSSGNWHCDSQWLGDRVITTSTRTWALP |
| | TYNNHLYKQISNSTSGGSSNDNAYFGYSTPWGYFDFNRFHCHFSPRDWQR |
| | LINNNWGFRPKRLNFKLFNIQVKEVTDNNGVKTIANNLTSTVQVFTDSDY |
| | QLPYVLGSAHEGCLPPFPADVFMIPQYGYLTLNDGSQAVGRSSFYCLEYF |
| | PSQMLRTGNNFQFSYEFENVPFHSSYAHSQSLDRLMNPLIDQYLYYLSKT |
| | INGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSAQAQAQTGWVQNQG |
| | ILPGMVWQDRDVYLQGPIWAKIPHTDGNFHPSPLMGGFGMKHPPPQILIK |
| | NTPVPADPPTAFNKDKLNSFITQYSTGQVSVEIEWELQKENSKRWNPEIQ |
| | YTSNYYKSNNVEFAVNTEGVYSEPRPIGTRYLTRNL |
| SEQ ID | MAADGYLPDWLEDNLSEGIREWWDLKPGAPKPKANQQKQDDGRGLVLPGY |
| NO: 62 | KYLGPFNGLDKGEPVNAADAAALEHDKAYDQQLKAGDNPYLRYNHADAEF |
| (AAV10 | QERLQEDTSFGGNLGRAVFQAKKRVLEPLGLVEEAAKTAPGKKRPVEPSP |
| (AAT46337)) | QRSPDSSTGIGKKGQQPAKKRLNFGQTGESESVPDPQPIGEPPAGPSGLG |
| | SGTMAAGGGAPMADNNEGADGVGSSSGNWHCDSTWLGDRVITTSTRTWAL |
| | PTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSE |
| | YQLPYVLGSAHQGCLPPFPADVFMIPQYGYLTLNNGSQAVGRSSFYCLEY |
| | FPSQMLRTGNNFEFSYTFEDVPFHSSYAHSQSLDRLMNPLIDQYLYYLSR |
| | TQSTGGTQGTQQLLFSQAGPANMSAQAKNWLPGPCYRQQRVSTTLSQNNN |
| | SNFAWTGATKYHLNGRDSLVNPGVAMATHKDDEERFFPSSGVLMFGKQGA |
| | GRDNVDYSSVMLTSEEEIKTTNPVATEQYGVVADNLQQANTGPIVGNVNS |
| | QGALPGMVWQNRDVYLQGPIWAKIPHTDGNFHPSPLMGGFGLKHPPPQIL |
| | IKNTPVPADPPTTFSQAKLASFITQYSTGQVSVEIEWELQKENSKRWNPE |
| | IQYTSNYYKSTNVDFAVNTEGTYSEPRPIGTRYLTRNL |
| SEQ ID | MAADGYLPDWLEDNLSEGIREWWALKPGAPQPKANQQHQDNARGLVLPGY |
| NO: 63 | KYLGPGNGLDKGEPVNEADAAALEHDKAYDQQLKAGDNPYLKYNHADAEF |
| (AAV | QERLKEDTSFGGNLGRAVFQAKKRLLEPLGLVEEAAKTAPGKKRPVEQSP |
| hu.68) | QEPDSSVGIGKSGAQPAKKRLNFGQTGDTESVPDPQPIGEPPAAPSGVGS |
| | LTMASGGGAPVADNNEGADGVGSSSGNWHCDSQWLGDRVITTSTRTWALP |
| | TYNNHLYKQISNSTSGGSSNDNAYFGYSTPWGYFDFNRFHCHFSPRDWQR |
| | LINNNWGFRPKRLNFKLFNIQVKEVTDNNGVKTIANNLTSTVQVFTDSDY |
| | QLPYVLGSAHEGCLPPFPADVFMIPQYGYLTLNDGSQAVGRSSPYCLEYF |
| | PSQMLRTGNNFQFSYEFENVPFHSSYAHSQSLDRLMNPLIDQYLYYLSKT |
| | INGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSAQAQAQTGWVQNQG |
| | ILPGMVWQDRDVYLQGPIWAKIPHTDGNFHPSPLMGGFGMKHPPPQILIK |
| | NTPVPADPPTAFNKDKLNSFITQYSTGQVSVEIEWELQKENSKRWNPEIQ |
| | YTSNYYKSNNVEFAVNTEGVYSEPRPIGTRYLTRNL* |
| SEQ ID | MAADGYLPDWLEDNLSEGIREWWALQPGAPKPKANQQHQDNARGLVLPGY |
| NO: 64 | KYLGPGNGLDKGEPVNAADAAALEHDKAYDQQLKAGDNPYLKYNHADAEF |
| (AAV | QERLKEDTSFGGNLGRAVFQAKKRLLEPLGLVEEAAKTAPGKKRPVDQSP |
| LK03) | QEPDSSSGVGKSGKQPARKRLNFGQTGDSESVPDPQPLGEPPAAPTSLGS |
| | NTMASGGGAPMADNNEGADGVGNSSGNWHCDSQWLGDRVITTSTRTWALP |
| | TYNNHLYKQISSQSGASNDNHYFGYSTPWGYFDFNRFHCHFSPRDWQRLI |
| | NNNWGFRPKKLSFKLFNIQVKEVTQNDGTTTIANNLTSTVQVFTDSEYQL |
| | PYVLGSAHQGCLPPFPADVFMVPQYGYLTLNNGSQAVGRSSFYCLEYFPS |
| | QMLRTGNNFQFSYTFEDVPFHSSYAHSQSLDRLMNPLIDQYLYYLNRTQG |
| | TTSGTTNQSRLLFSQAGPQSMSLQARNWLPGPCYRQQRLSKTANDNNNSN |
| | FPWTAASKYHLNGRDSLVNPGPAMASHKDDEEKFFPMHGNLIFGKEGTTA |
| | SNAELDNVMITDEEEIRTTNPVATEQYGTVANNLQSSNTAPTTRTVNDQG |
| | ALPGMVWQDRDVYLQGPIWAKIPHTDGHFHPSPLMGGFGLKHPPPQIMIK |
| | NTPVPANPPTTFSPAKFASFITQYSTGQVSVEIEWELQKENSKRWNPEIQ |
| | YTSNYNKSVNVDFTVDINGVYSEPRPIGTRYLTRPL* |
| SEQ ID | MAADGYLPDWLEDTLSEGIRQWWKLKPGPPPPKPAERHKDDSRGLVLPGY |
| NO: 65 | KYLGPFNGLDKGEPVNEADAAALEHDKAYDRQLDSGDNPYLKYNHADAEF |
| (AAV | QERLKEDTSFGGNLGRAVFQAKKRVLEPLGLVEEPVKTAPGKKRPVEHSP |
| hu.1 | VEPDSSSGTGKAGQQPARKRLNFGQTGDADSVPDPQPLGQPPAAPSGLGS |
| (AAS99260)) | TTMATGSGAPMADNNEGADGVGNSSGNWHCDSQWLGDRVITTSTRTWALP |
| | TYNNHLYKQISSQSGASNDNHYFGYSTPWGYFDFNRFHCHFSPRDWQRLI |
| | NNNWGFRPKRLNFKLENIQVKEVTQNGGTTTIANNLTSTVQVFTDSEYQL |
| | PYVLGSAHQGCLPPFPADVFMVPQYGYLTLNNGSQAVGRSSFYCLEYFPS |
| | QMLRTGNNFQFSYTFEDVPFHSSYAHSQSLDRLMNPLIDQYLYYLNKTQT |
| | NSGTLQQSRLLFSQAGPTNMSLQAKNWLPGPCYRQQRLSKQANGNNNSNF |
| | PWTAATKYHLNGRDSLVNPGPAMASHKDDEEKFFPMHGTLIFGKQGTNAN |
| | DADLENVMITDEEEIRATNPVATEQYGTVSNNLQNSNTGPTTGTVNHQGA |
| | LPGMVWQDRDVYLQGPIWAKIPHTDGHFHPSPLTGGFGLKHPPPQIMIKN |
| | TPVPANPPTNFSSAKFASFITQYSTGQVSVEIEWELQKENSKRWNPEIQY |
| | TSNYNKSVNVDFTVDINGVYSEPRPIGTRYLTRNL |
| SEQ ID | MAADGYPPDWLEDTLSEGIRQWWKLKPGPPPPKPAERHKDDSRGLVLPGY |
| NO: 66 | KYLGPFNGLDKGEPVNEADAAALEHDKAYDRQLDSGDNPYLKYNHADAEF |
| (AAV | QERLKEDTSFGGNLGRAVFQAKKRVLEPLGLVEEPVKTAPGKKRPVEHSP |
| hu.2 | VEPDSSSGTGKAGQRPARKRLNFGQTGDADSVPDPQPLGQPPAAPSGLGS |
| (AAS99270)) | TTMATGSGAPMADNNEGADGVGNSSGNWHCDSQWLGDRVITTSTRTWALP |
| | TYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQL |
| | PYVLGSAHQGCLPPFPADVFMVPQYGYLTLNNGSQAVGRSSFYCLEYFPS |
| | QMLRTGNNFQFSYTFEDVPFHSSYAHSQSLDRLMNPLIDQYLYYLNKTQT |
| | NSGTLQQSRLLFSQAGPTNMSLQAKNWLPGPCYRQQRLSKQANDNNNSNF |
| | PWTAATKYHLNGRDSLVNPGPAMASHKDDEEKFFPMHGTLIFGKQGTNAN |
| | DADLENVMITDEEEIRATNPVATEQYGTVSNNLQNSNTGPTTGTVNRQGA |
| | LPGMVWQDRDVYLQGPIWAKIPHTDGHFHPSPLMGGFGLKHPPPQIMIKN |
| | TPVPANPPTNFSSAKFASFITQYSTGQVSVEIEWELQKENSKRWNPEIQY |
| | TSNYNKSVNVDFTVDINGVYSEPRPIGTRYLTRNL |
| SEQ ID | MAADGYLPDWLEDTLSEGIRQWWKLKPGPPPPKPAERHKDDSRGLVLPGY |
| NO: 67 | KYLGPFNGLDKGEPVNEADAAALEHDKAYDRQLDSGDNPYLKYNHADAEF |
| (AAV | QERLKEDTSFGGNLGRAVFQAKKRVLEPLRPGLRKPVKTAPGKKRPVEHS |
| hu.3 | PVEPDSSSGTGKAGQQPARKRLNFGQTGDADSVPDPQPLGQPPAAPSGLG |
| (AAS99280)) | STTMATGSGAPMADNNEGADGVGNSSGNWHCDSQWLDDRVIATSTRTWAL |
| | PTYNNHLYKQISSQSGACNDNHYFGYSTPWGYFDFNRFHCHFSPRDWQRL |
| | INSNWGFRPKRLNFKLFNIQVKEVTQNDGTTTIANNLTSTVQVFTDSEYQ |
| | LPYVPGSAHQGCLPPFPADVFMVPQYGYLTLNNGSQAVGRSSFYCLEYFP |
| | SQMLRTGNNFQFSYTFEDVPFHSSYAHCQSLDRLMNPLIDQYLYYLNKTQ |
| | TNSGTLQQSRLLFSQAGPTNMSLQAKNWLPGPCYRQQRLSKQANDNNNCN |
| | FPWTAATKYHLNGRDSLVNPGPAMASHKDDEEKFFPMHGTLIFGKQGTNA |
| | NDADLENVMITDEEEIRPTNPVATEQYGTVSNNLQNSNTGPTTGTVNHQG |
| | ALPGMVWQDRDVYLQGPIWAKIPHTDGHFHPSPLMGGFGLKHPPPQIMIK |
| | STPVPANPPTNFSSAKFASSITQYSTGQVSVEIEWELQKENSKRWNPEIQ |
| | YTSNYNKSVNVDFTVDTNGVYSEPRPIGTRYLTRNL |
| SEQ ID | MAADGYLPDWLEDTLSEGIRQWWKLKPGPPPPKPAERHKDDSRGLVLPGY |
| NO: 68 | KYLGPFNGLDKGEPVNEADAAALEHDKAYDRQLDSGDNPYLKYNHADAEF |
| (AAV | QERLKEDTSFGGNLGRAVFQAKKRVLEPLGLVEEPVKTAPGKKRPVEHSP |
| hu.4 | VEPDSSSGTGKAGQQPARKRLNFGQTGDADSVPDPQPLGQPPAAPSGLGS |
| (AAS99 | TTMATGSGAPMADNNEGADGVGNSSGNWHCDSQWLGDRVITTSTRTWALP |
| 287)) | TYNNHLYKQISSQSGASNDNHYFGYSTPWGYFDFNRFHCHFSPRDWQRLV |
| | NNNRGFRPKRLNFKLFNIQVKEVTQNDGTTTIANNLTSTVQVFTDSEYQL |
| | PYVLGSAHQGCLPPFPADVFMVPQYGYLTLNNGSQAVGRSSFYCLEYFPS |
| | QMLRTGNNFQFSYTFEDVPFHSSYAHSQSLDRLMNPLIDQYLYYLNKTQT |
| | NSGTLQQSRLLFSQAGPTNMSLQAKNWLPGPCYRQQRLSKQANDNNNSNF |
| | PWTAATKYHLNGRDSLVNPGPAMASHKDDEEKFFPMHGTLIFGKQGTNAN |
| | DADLENVMITDEEEIRATNPVATEQYGTVSNNLQNSNTGPTTGTVNHQGA |
| | LPGMVWQDRDVYLQGPIWAKIPHTDGHFHPSPLMGGFGLKHPPPQIMIKN |
| | TPVPANPPTNFSSAKFASFITQYSTGQVSVEIEWELQKENSKRWNPEIQY |
| | TSNYNKSVNVDFTVDINGVYSEPRPIGTRYLTRNL |
| SEQ ID | MAADGYLPDWLEDNLSEGIREWWDLKPGAPKPKANQQKQDDGRGLVLPGY |
| NO: 69 | KYLGPFNGLDKGEPVNAADAAALEHDKAYDQQLKAGDNPYLRYNHADAEF |
| (AAV | QERLQEDTSFGGNLGRAVFQAKKRVLEPLGLVEEGAKTAPGKKRPVEPSP |
| hu.6 | QRSPDSSTGIGKTGQQPAKKRLNFGQTGDSESVPDPQPIGEPPAGPSGLG |
| (AAS99306)) | SGTMAAGGGAPMADNNEGADGVGSSSGNWHCDSAWLGDRVITTSTRPWAL |
| | PTYNNHLYKQISNGTSGGSTNDNTYFGYSTPWGYFDFNRFHCHFSPRDWQ |
| | RLINNNWGFRPKRLNFKLFNIQVKEVTQNEGTKTIANNLTSTIQVFTDSE |
| | YQLPYVLGSAHQGCPPPFPADVFMIPQYGYLTLNNGSQAVGRSSFYCLEY |
| | FPSQMRRTGNNFEFSYQFEDVPFHSSYAHSQSLDRLMNPLIDQYLYYLSR |
| | TQSTGGTAGTQQLLFSQAGPNNMSAQAKNWLPGPCYRQQRVSTTLSQNNN |
| | SNFAWTGATKYHLNGRDSLVNPGVAMATHKDDEERFFPSSGVLMFGKQGA |
| | GKDNVDYSSVMLTSEEEIKTTNPVATEQYGVVADNLQQQNAAPIVGAVNS |
| | QGALPGMVWQNRDVYLQGPIWAKIPHTDGNFHPSPLMGGFGLKHPPPQIL |
| | IKNTPVPADPPTTFSQAKLASFITQYSTGQVSVEIEWELQKENSKRWNPE |
| | IQYTSNYYKSTNVDFAVNTEGTYSEPRPIGTRYLTRNL |
| SEQ ID | MAADGYLPDWLEDTLSEGIRQWWKLKPGPPPPKPAERHKDDSRGLVLPGY |
| NO: 70 | KYLGPFNGLDKGEPVNEADAAALEHDKAYDRQLDSGDNPYLKYNHADAEF |
| (AAV | QERLKEDTSFGGNLGRAVFQAKKRVLEPLGLVEGPVKTAPGKKRPVEHSP |
| hu.7 | AEPDSSSGTGKAGQQPARKRLNFGQTGDADSVPDPQPLGQPPAAPSGLGS |
| (AAS99313)) | TTMATGSGAPMADNNEGADGVGNSSGNWHCDSQWLGDRVITTSTRTWALP |
| | TYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQL |
| | PYVLGSAHQGCLPPFPADVFMVPQYGYLTLNNGSQAVGRSSFYCLEYFPS |
| | QMLRTGNNFQFSYTFEDVPFHSSYAHSQSLDRLMNPLIDQYLYYLNKTQS |
| | NSGTLQQSRLLFSQAGPTSMSLQAKNWLPGPCYRQQRLSKQANDNNNSNF |
| | PWTAATKYHLNGRDSLVNPGPAMASHKDDEEKFFPMHGTLIFGKQGTNAN |
| | DADLDNVMITDEEEIRTTNPVATEQYGYVSNNLQNSNTGPTTGTVNHQGA |
| | LPGMVWQDRDVYLQGPIWAKIPHTDGHFHPSPLMGGFGLKHPPPQIMIKN |
| | TPVPANPPTNFSSAKFASFITQYSTGQVSVEIEWELQKENSKRWNPEIQY |
| | TSNYNKSVNVDFTVDINGVYSEPRPIGTRYLTRNL |
| SEQ ID | MAADGYLPDWLEDTLSEGIRQWWKLKPGPPPPKPAERHQDNSRGLVLPGY |
| NO: 71 | KYLGPSNGLDKGEPVNEADAAALEHDKAYDRQLDSGDNPYLKYNHADAEF |
| (AAV | QERLKEDTSFGGNLGRAVFQAKKRVLEPLGLVEEPVKTAPGKKRPVEHSP |
| hu.9 | VEPDSSSGTGKAGHQPARKRLNFGQTGDADSVPDPQPLGQPPAAPTSLGS |
| (AAS99314)) | TTMATGSGAPMADNNEGADGVGNSSGNWHCDSQWLGDRVITTSTRTWALP |
| | TYNNHLYKQISSQSGASNDNHYFGCSTPWGYFDFNRFHCHFSPRDWQRLI |
| | NNNWGFRPKRLNFKLFNIQVKEVTQNDGTTTIANNLTSTVQVFTDSEYPL |
| | PYVLGSAHQGCLPPFPADVFMVPQYGYLTLNNGSQAVGRSSFYCLEYFPS |
| | QMLRTGNNFTFSYTFEDVPFHSSYAHSQSLDRLMNPLIDQYLYYLNRTQS |
| | NSGTLQQSRLLESQAGPTSMSLQAKNWLPGPCYRQQRLSKQANDNNNSNF |
| | PWTAATKYHLNGRDSLVNPGPAMASHKDDEEKFFPMHGTLIFGKQGTNAN |
| | DADLEHVMITDEEEIRTTNPVATEQYGNVSNNLQNSNTGPTTENVNHQGA |
| | LPGMVWQDRDVYLQGPIWAKIPHTDGHFHPSPLMGGFGLKHPPPQIMIKN |
| | TPVPANPPTNFSSAKFASFITQYSTGQVSVEIEWELQKENSKRWNPEIQY |
| | TSNYNKSVNVDFTVDINGVYSEPCPIGTRYLTRNL |
| SEQ ID | MAADGYLPDWLEDTLSEGIRQWWKLKPGPPPPKLAERHQDDSRGLVLPGY |
| NO: 72 | KYLGPFNGLDKGEPVNEADAAALEHDKAYDRQLDSGDNPYLKYNHADAEF |
| (AAV | QERLKEDTSFGGNLGRAVFQAKKRVLEPLGLVEEPVKTAPGKKRPVEHSP |
| hu.10 | VEPDSSSGTGKAGHQPARKRLNFGQTGDADSVPDPQPLGQPPAAPTSLGS |
| (AAS99261)) | TTMATGSGAPMADNNEGADGVGNSSGNWHCDSQWLGDRVITTSTRTWALP |
| | TYNNHLYKQISSQSGASNDNHYFGYSTPWGYFDFNRFHCHFSPRDWQRLI |
| | NNNWGFRPKRLNFKLENIQVKEVTQNDGTTTIANNLTSTVQVFTDSEYQL |
| | PYVLGSAHQGCLPPFPADVFTVPQYGYLTLNNGSQAVGRSSFYCLEYFPS |
| | QMLRTGNNLTFSYTFEDVPFHSSYAHSQSLDRLMNPLIDQYLYYLNRTQS |
| | NSGTLQQSRLLFSQAGPTSMSLQAKNWLPGPCYRQQRLSKQANDNNNSNF |
| | PWTAATKYHLNGRDSLVNPGPAMASHKDDEEKFFPMHGTLIFGKQGTNAN |
| | DADLEHVMITDEEEIRTTNPVATEQYGNVSNNLQNSNTGPTTENVNHQGA |
| | LPGMVWQDRDVYLQGPIWAKIPHTDGHFHPSPLMGGFGLKHPPPQIMIKN |
| | TPVPANPPTNYSSAKFASFITQYSTGQVSVEIEWELRKENSKRWNPEIQY |
| | TSNYNKSVNVDFTVDINGVYSEPRPIGTRYLTRNL |
| SEQ ID | MAADGYLPDWLEDTLSEGIRQWWKLKPGPPPPKPAERHQDDSRGLVLPGY |
| NO: 73 | KYLGPFNGLDKGEPVNEADAAALEHDKAYDRQLDSGDNPYLKYNHADAEF |
| (AAV | QERLKEDTSFGGNLGRAVFQAKKRVLEPLGLVEEPVKTAPGKKRPVEHSP |
| hu.11 | VEPDSSSGTGKAGHQPARKRLNFGQTGDADSVPDPQPLGQPPAAPTSLGS |
| (AAS99262)) | TTMATGSGAPMADNNEGADGVGNSSGNWHCDSQWLGDRVITTSTRTWALP |
| | TYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQL |
| | PYVLGSAHQGCLPPFPADVFMVPQYGYLTLNNGSQAVGRSSFYCLEYFPS |
| | QMLRTGNNFTFSYTFEDVPFHSSYAHSQSLDRLMNPLIDQYLYYLNRTQS |
| | NSGTLQQSRLLFSQAGPTSMSLQAKNWLPGPCYRQQRLSKQANDNNNSNF |
| | PWTAATKYRLNGRDSLVNPGPAMASHKDDEEKFFPMHGTLIFGKQGTNAN |
| | DADLEHVMITDEEEIRTTNPVATEQYGNVSNNLQNSNTGPTTENVNHQGA |
| | LPGMVWQDRDVYLQGPIWAKIPHTDGHFHPSPLMGGFGLKHPPPQIMIKN |
| | TPVPANPPTNFSSAKFASFITQYSTGQVSVEIEWELQKENSKRWNPEIQY |
| | TSNYNKSVNVDFTVDTNGVYSEPRPIGTRYLTRNL |
| SEQ ID | MAADGYLPDWLEDTLSEGIRQWWKLKPGPPPPKPAERHKDDSRGLVLPGY |
| NO: 74 | KYLGPFNGLYKGEPVDEADAAALEHDKAYDRQLDSGDNPYLKYNHADAEF |
| (AAV | QERLKEDTSFGGNLGRAVFQAKKRVLEPLGLVGEPVKTAPGKKRPVEHSP |
| hu.15 | VEPDSSSGTGKAGNQPARKRLNFGQTGDADSVPDPQPLGQPPAAPSGLGS |
| (AAS99265)) | TTMATGSGAPVADNNEGADGVGNSSGNWHCDSQWLGDRVITTSTRTWALP |
| | TYNNHLYKQISSQSGASNDNHYFGYSTPWGYFDFNRFHCHFSPRDRQRLI |
| | NNNWGFRPKRLNFKLFNIQVKEVTQNDGTTTIANNLTSTVQVFTDSGYQL |
| | PYVLGLAHQGCLPPFPADVFMVPQYGYLTLNNGSQAVGRSSFYCLEYFPS |
| | QMLRTGNNFQFSYTFEDVPFHSSYAHSQSLDRLMNPLIDQYLYYLNKTQS |
| | NSGTLQQSRLLFSQAGPTSMSLQAKNWLPGPCYRQQRLSKQANDNNNSNF |
| | PWTAATKYHLNGRDSLVNPGPAMASHKDDEEKFFPMHGTLIFGKQGTNAN |
| | DADLDNVMITDEEEIRTTNPVATEQYGYVSNNLQNSNTGPTTGTVNHQGA |
| | LPGMVWQDRDVYLQGPIWAKIPHTDGHFHPSPLMGGFGLKHPPPQIMIKN |
| | TPVPANPPTNFSSAKFASFITQYSTGQVSVEIEWELQKEDSKRWNPEIQY |
| | TSNYNKPVNVDFTVDINGVYSEPRPIGTRYLTRNL |
| SEQ ID | MAADGYLPDWLEDTLSEGIRQWWKLKPGPPPPKPAERHKDDSRGLVLPGY |
| NO: 75 | KYLGPFNGLYKGEPVNEADAAALEHDKAYDRQLDSGDNPYLKYNHAGAEF |
| (AAV | QERLKEDTSFGGNLGRAVFQAKKRVLEPLGLVEEPVKTAPGKKRPVEHSP |
| hu.16 | VEPDSSSGTGKAGNQPARKRLNFGQTGDADSVPDPQPLGQPPAAPSGLGS |
| (AAS99266)) | TTMATGSGAPVADNNEGADGVGNSSGNWHCDSQWLGDRVITTSTRTWALP |
| | TYNNHLYKQISSQSGASNDNHYFGYSTPWGYFDFNRFHCHFSPRDWQRLI |
| | NNNWGFRPKRLNFKLENIQVKEVTQNDGTTTIANNLTSTVQVFTDSEYQL |
| | PYVLGSAHQGCLPPFPADVEMVPQYGYLTLNNGSQAVGRSSFYCLEYFPS |
| | QMLRTGNNFQFSYTFEDVPFHSSYAHSQSLDRLMNPLIDQYLYYLNKTQS |
| | NSGTLQQSRLLFSQAGPTSMSLQAKNWLPGPCYRQQRLSKQANDNNNSNF |
| | PWTAATKYHLNGRDSLVNPGPAMASHKDDEEKFFPMHGTLIFGKQGTNAN |
| | DADLDNVMITDEEEIRTTNPVATEQYGYVSNNLQDSNTGPTTGTVNHQGA |
| | LPGMVWQDRDVYLQGPIWAKIPHTDGHFHPSPLMGGFGLKHPPPQIMIKN |
| | TPVPANPPTNFSSAKFASFITQYSTGQVSVEIEWELQKENSKRWNPEIQY |
| | TSNYNKSVNVDFTVDINGVYSEPRPIGTRYLTRNL |
| SEQ ID | MAADGYLPDWLEDNLSEGIREWWDLKPGAPKPKANQQKQDDGRGLVLPGC |
| NO: 76 | KYLGPFNGLDKGEPVNAADAAALEHDKAYDQQLKAGDNPYLRYNHADAEF |
| (AAV | QERLQEDTSFGGNLGRAVFQAKKRVLEPLGLVEEGAKTAPGKKRPVEPSP |
| hu.17 | QRSPDSSTGIGKTGQQPAKKRLNFGQTGDSESVPDPQPIGEPPAGPSGLG |
| (AAS99267)) | SGTMAAGGGAPMADNNEGADGVGSSSGNWHCDSTWLGDRVITTSTRTWAL |
| | PTYNNHLYKQISNGTSGGSTNDNTYFGYSTPWGYFDFNRFHCHFSPRDWQ |
| | RLINNNWGFRPKRLNFKLFNIQVKEVTQNEGTKTIANNLTSTIQVFTDSE |
| | YQLPYVLGSAHQGCPPPFPADVFMIPQYGYLTLNNGSQAVGRSSFYCLEY |
| | FPSQMRRTGNNFEFSYQFEDVPFHSSYAHSQSLDRLMNPLIDQYLYYLSR |
| | TQSTGGTAGTQQLLFSQAGPNNMSAQAKNWLPGPCYRQQRVSTTLSQNNN |
| | SNFAWTGATKYHLNGRDSLVNPGVAMATHKDDEERFFPSSGVLMFGKQGA |
| | GKDNVDYSSVMLTSEEEIKTTNPVATEQYGVVADNLQQQNAAPIVGAVNS |
| | QGALPGMVWQNRDVYLQGPIWAKIPHTDGNFHPSPLMGGFGLKHPPPQIL |
| | IKNTPVPADPPTTFSQAKLASFITQYSTGQVSVEIEWELQKENSKRWNPE |
| | IQYTSNYNKSVNVDFTVDINGVYSEPRPIGTRYLTRNL |
| SEQ ID | MAADGYLPDWLEDTLSEGIRQWWKLKPGPPPPKPAERHKDDSRGLVLPGY |
| NO: 77 | KYLGPFNGLDKGEPVNEADAAALEHDKAYDRQLESGDNPYLKYNHADAEF |
| (AAV | QERLKEDTSFGGNLGRAVFQAKKRVLEPLGLVEEPVKTAPGKKRPVEHSP |
| hu.18 | VEPDSSSGTGKAGQQPARKRLNFGQTGDADSVPDPQPLGQPPAAPSGLGS |
| (AAS99268)) | TTMASGSGAPVADNNEGADGVGNSSGNWHCDSQWLGDRVITTSTRTWALP |
| | TYNNHLYKQISSQSGASNDNHYFGYSTPWGYFDFNRFHCHFSPRDWQRLI |
| | NNSWGFRPKRLNFKLFNIQVKEVTQNDGTTTIANNLTSTVQVFTDSEYQL |
| | PYVLGSAHQGCLPPFPADVFMVPQYGYLTLNNGSQAVGRSSFYCLEYFPS |
| | QMLRTGNNFQFSYTFEDVPFHSSYAHSQSLDRLLNPLIDQYLYYLNKTQS |
| | NSGTLQQSRLLFSQAGPTSMSLQAKNWLPGPCYRQQRLSKQANDNNNSNF |
| | PWTAATKYHLNGRDSLVNPGPAMASHKDDEEKFFPMHGTLIFGKQGTNAN |
| | DADLDNVMITDEEEIRTTNPVATEQYGYVSNNLQNSNTGPTTGTVNHQGA |
| | LPGMVWQDRDVYLQGPIWAKIPHTDGHFHPSPLMGGFGLKHPPPQIMIKN |
| | TPVPANPPTNFSSSKFASFITQYSTGQVSVEIEWELQKENSKRWNPEIQY |
| | TSNYNKSVNVDFTVDINGVYSEPRPIGTRYPTRNL |
| SEQ ID | MAADGYLPDWLEDTLSEGIRQWWKLKPGPPPPKPAERHKDDSRGLVLPGY |
| NO: 78 | RYLGPFNGLDKGEPVNEADAAALEHDKAYDRQLDSGDNPYLKYNHVDAEF |
| (AAV | QERLKEDTSFGGNLGRAVFQAKKRILEPLGLVEEPVKAAPGEKRPVEHSP |
| hu.20 | AEPDSSSGTGKAGQQPARKRLNFGQTGDADSVPDPQPLGQPPAAPSGLGT |
| (AAS99271)) | NTMASGSGAPMADNNEGADGVGNSSGNWHCDSTWMGDRVITTSTRTWALP |
| | TYNNHLYKQISSQSGASNDNHYFGYSTPWGHFDFNRFHCHFSPRDWQRLI |
| | NNNWGFRPKRLSFKLFNIQVKEVTQNDGTTTIANNLTSTVQVFTDSEYQL |
| | PYVLGSAHQGCLPPFPADVFMVPQYGYLTLNNGSQAVGRSSFYCLEYFPS |
| | QMLRTGNNFTFSYTFEDVPFHSSYAHSQSLDRLMNPLIDQYLYYLSRTNT |
| | PSGTTTMSRLQFSQAGASDIRDQSRNWLPGPCYRQQRVSKTAADNNNSDY |
| | SWTGATKYHLNGRDSLVNPGPAMASHKDDEEKYFPQSGVLIFGKQDSGKT |
| | NVDIEKVMITDEEEIRTTNPVATEQYGSVSTNLQSGNTQAATSDVNTQGV |
| | LPGMVWQDRDVYLQGPIWAKIPHTDGHFHPSPPMGGFGLKHPPPQILIKN |
| | TPVPANPSTTFSAAKFASFITQYSTGQVSVEIEWELQKENSKRWNPEIQY |
| | TSNYNKSVNVDFTVDINGVYSEPRPIGARYLTRNL |
| SEQ ID | MAADGYLPDWLEDTLSEGIRQWWKLKPGPPPPKPAERHKDDSRGLVLPGY |
| NO: 79 | KYLGPFNGLDKGEPVNEADAAALEHDKAYDRQLDSGDNPYLKYNHADAEF |
| (AAV | QERLKEDTSFGGNLGRAVFQAKKRILEPLGLVEEPVKTAPGKKRPVEHSP |
| hu.21 | AEPDSSSGTGKAGQQPARKRLNFGQTGDADSVPDPRPLGQPPAAPSGLGT |
| (AAS99272)) | NTMASGSGAPMADNNEGADGVGNSSGNWHCDSTWMGDRVITTSTRTWALP |
| | TYNNHLYKQISSQSGASNDNHYFGYSTPWGYFDFNRFHCHFSPRDWQRLI |
| | NNNWGFRPKRLSFKLFNIQVKEVTQNDGTTTIANNLTSTVQVFTDSEYQL |
| | PYVLGSAHQGCLPPFPADVFMVPQYGYLTLNNGSQAVGRSSFYCLEYFPS |
| | QMLRTGNNFTFSYTFEDVPFHSSYAHSQSLDRLMNPLIDQYLYYLSRTNT |
| | PSGTTTMSRLQFSQAGASDIRDQSRNWLPGPCYRQQRVSKTAADNNNSDY |
| | SWTGATKYHLNGRDSLVNPGPAMASHKDDEEKYFPQSGVLIFGKQDSGKT |
| | NVDIEKVMITDEEEIRTTNPVATEQYGSVSTNLQSGNTQAATSDVNTQGV |
| | LPGMVWQDRDVYLQGPIWAKIPHTDGHFHPSPLMGGFGLKHPPPQILIKN |
| | TPVPANPSTTFSAAKFASFITQYSTGQVSVEIEWELQKENSKRWNPEIQY |
| | TSNYNKSVNVDFTVDINGVYSEPRPIGTRYLTRNL |
| SEQ ID | MAADGYLPDWLEDTLSEGIRQWWKLKPGPPPPKPAERHKDDSRGLVLPGY |
| NO: 80 | KYLGPFNGLDKGEPVNEADAAALEHDKAYDRQLDSGDNPYLKYNHADAEF |
| (AAV | QERLKGDTSFGGNLGRAVFQAKKRILEPLGLVEEPVKTAPGKKRPVEHSP |
| hu.22 | AEPDSSSGTGKAGQQPARKRLNFGQTGDADSVPDPQPLGQPPAAPSGLGT |
| (AAS99273)) | NTMASGSGAPMADNNEGADGVGNSSGNWHCDSTWMGGRVITTSTRTWALP |
| | TYNNHLYKQISSQSGASNDNHYFGYSTPWGYFDFNRFHCHFSPRDWQRLI |
| | NNNWGFRPKRLSFKLFNIQVKEVTQNDGTTTIANNLTSTVQVFTDSEYQL |
| | PYVLGSAHQGCLPPFPADVFMVPQYGYLTLNNGSQAVGRSSFYCLEYFPS |
| | QTLRTGNNFTFSYTFEDVPFHSSYAHSQSLDRLMNPLIDQYLYYLSRTNT |
| | PSGTTTMSRLQFSQAGASDIRDQSRNWLPGPCYRQQRVSKTAADNNNSDY |
| | SWTGATKYHLNGRDSLVNPGPAMASHKDDEEKYFPQSGVLIFGKQDSGKT |
| | NVDIEKVMITDEEEIRTTNPVATEQYGSVSTNLQSGNTQAATSDVNTQGV |
| | LPGMVWQDRDVYLQGPIWAKIPHTDGHFHPSPLMGGFGLKHPPPQILIKN |
| | TPVPANPSTTFSAAKFASFITQYSTGQVSVEIEWELQKENSKRWNPEIQY |
| | TSNYNKSVNVDFTVDINGVYSEPRPIGTRYLTRNL |
| SEQ ID | MAADGYLPDWLEDTLSEGIRQWWKLKPGPPPPKPAERHKDDSRGLVLPGY |
| NO: 81 | KYLGPFNGLDKGEPVNEADAAALEHDKAYDRQLDSGDNPYLKYNHADAEF |
| (AAV | QERLKEDTSFGGNLGRAVFQAKKRILEPLGLVEEPVKTAPGKKRPVEHSP |
| ho 23 | AEPDSSSGTGKAGQQPARKRLNFGQTGDADSVPDPQPLGQPPAAPSGLGT |
| (AAS99274)) | NTMASGSGAPMADNNEGADGVGNSSGNWHCDSTWMGDRVITTSTRTWALP |
| | TCNNHLYKQISSQSGASNDNHYFGYSTPWGYFDFNRFHCHFSPRDWQRLI |
| | NNNWGFRPKRLSFKLFNIQVKEVTQNDGTTTIANNLTSTVQVFTDSEYQL |
| | PYVLGSAHQGCLPPFPADVFMVPQYGYLTLNNGSQAVGRSSFYCLEYFPS |
| | QMLRTGNNFTFSYTFEDVPFHSSYAHSQSLDRLMNPLIDQYLYYLSRTNT |
| | PSGTTTMSRLQFSQAGASDIRDQSRNWLPGPCYRQQRVSKTAADNNNSDY |
| | SWTGATKYHLNGRDSLVNPGPAMASHKDDEEKYFPQSGVLIFGKQDSGKT |
| | NVDIEKVMITDEEEIRTTNPVATEQYGSVSTYLQSGNTQAATSDVNTQGV |
| | LPGMVWQDRDVYLRGPIWAKIPHTDGHFHPSPLMGGFGLKHPPPQILIKN |
| | TPVPANPSTTFSAAKFASFITQYSTGQVSVEIEWELQKENSKRWNPEIQY |
| | TSNYNKSVNVDFTVDTNGVYSEPRPIGTRYLTRNL |
| SEQ ID | MAADGYLPDWLEDTLSEGIRQWWKLKPGPPPPKPAERHKDGSRGLVLPGY |
| NO: 82 | KYLGPFNGLDKGEPVNEADAAALEHDKAYDRQLNSGDNPYLKYNHADAEF |
| (AAV | QERLKEDTSFGGNLGRAVFQAKKRVLEPLGLVEEPVKTAPGKKRPVEHSP |
| hu.25 | AEPDSSSGTGKAGQQPARKRLNFGQTGDADSVPDPQPLGQPPAAPSGLGS |
| (AAS99276)) | TTMATGSGAPMADNNEGADGVGNSSGNWHCDSQWLGDRVITTSTRTWALP |
| | TYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQL |
| | PYVLGSAHQGCLPPFPADVFMVPQYGYLTLNNGSQAVGRSPFYCLEYFPS |
| | QMLRTGNNFQFSYTFEDVPFHSSYAHSQSLDRLMNPLIDQYLYYLNKTQT |
| | NSGTLQQSRLLFSQAGPTNMSLQAKNWLPGPCYRQQRLSKQANDNNNSNF |
| | PWTAATKYHLNGRDSLVNPGPAMASHKDDEEKFFPMHGTLIFGKQGTNAN |
| | DADLENVMITDEEEIRTTNPVATEQYGTVSNNLQNSNTGPTTGTVNHQGA |
| | LPGMVWQDRDVYLQGPIWAKIPHTDGHFHPSPLMGGFGLKHPPPQIMIKN |
| | TPVPANPPTNFSSAKFASFITQYSTGQVSVEIEWELQKENSKRWNPEIQY |
| | TSNYNKSVNVDFTVDNNGVYSEPRPIGTRYLTRNL |
| SEQ ID | MAADGYLPDWLEDTLSEGIRQWWKLKPGPPPPKPAERHKDDSRGLVLPGY |
| NO: 83 | KYLGPFNGLDKGEPVNEADAAALEHDKAYDRQLDSGDNPYLKYNHADAEF |
| (AAV | QERLKEDTSFGGNLGRAVFQAKKRILEPLGLVEEPVKTAPGKKRPVEHSP |
| hu.27 | AEPDSSSGTGKAGQQPARKRLNFGQTGDADSVPDPQPLGQPPAAPSGLGT |
| (AAS99277)) | NTMASGSGAPMADNNEGADGVGNSSGNWHCDSTWMGDRVITTSTRTWALP |
| | TYNNHLYKQISSQSGASNDNHYFGYSTPWGYFDFNRFHCHFSPRDWQRLI |
| | NNNWGFRPKRLSFKLFNIQVKEVTQNDGTTTIANNLTSTVQVFTDSGYQL |
| | PYVLGSAHQGCLPPFPADVFMVPQYGYLTLNNGSQAVGRSSFYCLEYFPS |
| | QMLRTGNNFTFSYTFEDVPFHSSYAHGQSLDRLMNPLIDQYLYYLSRTNT |
| | PSGTTTMSRLQFSQAGASDVRDQSRNWLPGPCYRQQRVSKTAADNNNSDY |
| | SWTGATKYHLNGRDSLVNPGPAMASHKDDEEKYFPQSGVLVFGKQDSGKT |
| | NVDIEKVMITDEEEIRTINPAATEQYGSVSTNLQSGNTQAATSDVNTQGV |
| | LPGMVWQDRDVYLQGPIWAKIPHTDGHFHPSPLMGGFGLKHPPPQILIKN |
| | TPVPANPSTTFSAAKFVSFITQYSTGQVSVEIEWELQKENSKRWNPEIQY |
| | TSNYNKSVNVDFTVDINGVYSEPRPIGTRYLTRNL |
| SEQ ID | MAADGYLPDWLEDTLSEGIRQWWKLKPGPPPPKPAERHKDDSRGLVLPGY |
| NO: 84 | KYLGPFNGLDKGEPVNEADAAALEHDKAYDRQLDSGDNPYLKYNHADAEF |
| (AAV | QERLKEDTSFGGNLGRAVFQAKKRVLEPLSLVEEPVKTAPGKKRPVEHSP |
| hu.28 | AEPDSSSGTGKSGNQPARKRLNFGQTGDSDSVPDPQPLGQPPAAPSGLGT |
| (AAS99278)) | NTMATGSGAPMADNNEGADGVGNSSGNWHCDSTWMGDRVITTSTRTWALP |
| | TYNNHLYKQISSQSGASNDNHYFGYSTPWGYFDFNRFHCHFSPRDWQRLI |
| | NNNWGFRPKRLNFKLENIQVKEVTQNDGTTTIANNLTSTVQVFTDSEYQL |
| | PYVLGSAHQGCLPPFPADVFMVPQYGYLTLNNGSQAVGRSSFYCLEYFPS |
| | QMLRTGNNFTFSYTFEDVPFHSSYAHSQSLDRLMNPLIDQYLYYLSRTNT |
| | PSGTTTQSRLQFSQAGASDIQDQSRNWLPGPCYRQQRVSKTSADNNNSEY |
| | SWTGATKYHLNGRDSLVNPGPAMASHKDDEEKFFPQSGVLIFGKQGSEKT |
| | NVDIEKVMITDEEEIRTTNPVATEQYGSVSTNLQSGNTQAATADVNTQGV |
| | LPGMVGQDRDVYLQGPTWAKIPHTDGHFHPSPLMGGFGLKHPPPQILIKN |
| | TPVPANPSTTFSAAKFASFITQYSTGQVSVEIEWELQKENSKRWNPEIQY |
| | TSNYNKSVNVDFTVDTNGVYSEPRPIGTRYLTRNL |
| SEQ ID | MAADGYLPDWLEDTLSEGIRQWWKLKPGPPPPKPAERHKDDSRGLVLPGY |
| NO: 85 | KYLGPFNGLDKGEPVNEADAAALEHDKAYDRQLDSGDNPYLKYNHADAEF |
| (AAV | QERLKEDTSFGGNLGRAVFQAKKRVLEPLGLVEEPVKTAPGKKRPVEHSP |
| hu.29 | AEPDSSSGTGKSGNQPARKRLNFGQTGDSDSVPDPQPLGQPPAAPSGLGT |
| (AAS99279)) | NTMATGSGAPMADNNEGADGVGNSSGNWHCDSTWMGDRVITTSTRTWALP |
| | TYNNHLYKQISSQSGASNDNHYFGYSTPWGYFDFNRFHCHFSPRDWQRLI |
| | NNNWGFRPKRLNFKLENIQVKEVTQNDGTTTIANNLTSTVQVFTDSEYQL |
| | PYVLGSAHQGCLPPFPADVFMVPQYGYLTLNNGSQAVGRSSFYCLGYFPS |
| | QMLRTGNNFTFSYTFEDVPFHSSYAHSQSLDRLMNPLIDQYLYYLSRTNT |
| | PSGTTTQSRLQFSQAGASDIRDQSRNWLPGPCYRQQRVSKTSADNNNSEY |
| | SWTGATKYHLNGRDSLVNPGPAMASHKDDEEKFFPQSGVLIFGKQGPEKT |
| | NVDIEKVMITDEEEIRTTNPVATEQYGSVSTNLQSGNTQAATADVNTQGV |
| | LPGMVWQDRDVYLQGPIWAKIPHTDGHFHPSPLMGGFGLKHPPPQILIKN |
| | TPVPANPSTTFSAAKFASFITQYSTGQVSVEIEWELQKENSKRWNPEIQY |
| | TSNYNKSVNVDFTVDTNGVYSEPRPIGTRYLTRNL |
| SEQ ID | MAADGYLPDWLEDTLSEGIRQWWKLKPGPPPPKPAERHKDDSRGLVLPGY |
| NO: 86 | KYLGPGNGLDKGEPVNAADAAALEHDKAYDQQLKAGDNPYLKYNHADAEF |
| (AAV | QERLKEDTSFGGNLGRAVFQAKKRLLEPLGLVEEAAKTAPGKKRPVEQSP |
| hu.31 | QEPDSSAGIGKSGSQPAKKKLNFGQTGDTESVPDPQPIGEPPAAPSGVGS |
| (AAS99281)) | LTMASGGGAPVADNNEGADGVGSSSGNWHCDSQWLGDRVITTSTRTWALP |
| | TYNNHLYKQISNSTSGGSSNDNAYFGYSTPWGYFDFNRFHCHFSPRDWQR |
| | LINNNWGFRPKRLNFKLFNIQVKEVTDNNGVKTIANNLTSTVQVFTDSDY |
| | QLPYVLGSAHEGCLPPFPADVFMIPQYGYLTLNDGGQAVGRSSFYCLEYF |
| | PSQMLRTGNNFQFSYEFENVPFHSSYAHSQSLDRLMNPLIDQYLYYLSKT |
| | INGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSAQAQAQTGWVQNQG |
| | ILPGMVWQDRDVYLQGPIWAKIPHTDGNFHPSPLMGGFGMKHPPPQILIK |
| | NTPVPADPPTAFNKDKLNSFITQYSTGQVSVEIEWELQKENSKRWNPEIQ |
| | YTSNYYKSNNVEFAVSTEGVYSEPRPIGTRYLTRNL |
| SEQ ID | MAADGYLPDWLEDTLSEGIRQWWKLKPGPPPPKPAERHKDDSRGLVLPGY |
| NO: 87 | KYLGPGNGLDKGEPVNAADAAALEHDKAYDQQLKAGDNPYLKYNHADAEF |
| (AAV | QERLKEDTSFGGNLGRAVFQAKKRLLEPLGLVEEAAKTAPGKKRPVEQSP |
| hu.32 | QEPDSSAGIGKSGSQPAKKKLNFGQTGDTESVPDPQPIGEPPAAPSGVGS |
| (AAS99282)) | LTMASGGGAPVADNNEGADGVGSSSGNWHCDSQWLGDRVITTSTRTWALP |
| | TYNNHLYKQISNSTSGGSSNDNAYFGYSTPWGYFDFNRFHCHFSPRDWQR |
| | LINNNWGFRPKRLNFKLFNIQVKEVTDNNGVKTIANNLTSTVQVFTDSDY |
| | QLPYVLGSAHEGCLPPFPADVFMIPQYGYLTLNDGSQAVGRSSFYCLEYF |
| | PSQMLRTGNNFQFSYEFENVPFHSSYAHSQSLDRLMNPLIDQYLYYLSKT |
| | INGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSAQAQAQTGWVQNQG |
| | ILPGMVWQDRDVYLQGPIWAKIPHTDGNFHPSPLMGGFGMKHPPPQILIK |
| | NTPVPADPPTAFNKDKLNSFITQYSTGQVSVEIEWELQKENSKRWNPEIQ |
| | YTSNYYKSNNVEFAVNTEGVYSEPRPIGTRYLTRNL |
| SEQ ID | MAADGYLPDWLEDTLSEGIRQRWKLKPGPPPPEPAERHKDDSRGLVLPGY |
| NO: 88 | KYLGPFNGLDKGEPVNEADAAALEHDKAYDRQLDSGDNPYLKYNHADAEF |
| (AAV | QERLKEDTSFGGNLGRAVFQAKKRVLEPLGLVEEPVKTAPGKKRPVEHSP |
| hu.34 | VEPDSSSGTGKAGQQPARKRLNFGQTGDADSVPDPQPLGQPPAAPSGLGT |
| (AAS99283)) | NTMATGSGAPMADNNEGADGVGNSSGNWHCDSTWMGDRVITTSTRTWALP |
| | TYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQL |
| | PYVLGSAHQGCLPPFPADVFMVPQYGYLTLNNESQAVGRSSFYCLEYFPS |
| | QMLRTGNNFTFSYTFEDVPFHSSYAHSQSLGRLMNPLIDQYLYYLSRTNT |
| | PSGTTTQSRLQFSQAGASDIRDQSRNWLPGPCYRQQRVSKTSADNNNSEY |
| | SWTGATKYHLNGRDSLVNPGPAMASHKDDEEKFFPQSGVLIFGKQGSEKT |
| | NVDIEKVMITDEEEIRTTNPVATEQYGSVSTNLQRGNRQAATADVNTQGV |
| | LPGMVWQDRDVYLQGPIWAKIPHTDGHFHPSPLMGGFGLKHPPPQILIKN |
| | TPVPANPSTTFSAAKFASFITQYSTGQVSVEIEWELQKENSKRWNPEIQY |
| | TSNYNKSVNVDFTVDTNGVYSEPRPIGTRYLTRNL |
| SEQ ID | MAADGYLPDWLEDNLSEGIREWWDLKPGAPKPKANQQKQDDGRGLVLPGY |
| NO: 89 | KYLGPFNGLDKGEPVNAADAAALEHDKAYDQQLKAGDNPYLRYNHADAEF |
| (AAV | QERLQEDTSFGGNLGRAVFQAKKRVLEPLGLVEEAAKTAPGKKRPVEPSP |
| hu.37 | QRSPDSSTGIGKKGQQPAKKRLNFGQTGDSESVPDPQPIGEPPAGPSGLG |
| (AAS99285)) | SGTMAAGGGAPMADNNEGADGVGSSSGNWHCDSTWLGDRVITTSTRTWAL |
| | PTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSE |
| | YQLPYVLGSAHQGCLPPFPADVFMIPQYGYLTLNNGSQAVGRSSFYCLEY |
| | FPSQMLRTGNNFEFSYTFEDVPFHSSYAHSQSLDRLMNPLIDQYLYYLSR |
| | TQSTGGTQGTQQLLFSQAGPANMSAQAKNWLPGPCYRQQRVSTTLSQNNN |
| | SNFAWTGATKYHLNGRDSLVNPGVAMATHKDDEERFFPSSGVLMFGKQGA |
| | GRDNVDYSSVMLTSEEEIKTTNPVATEQYGVVADNLQQTNTGPIVGNVNS |
| | QGALPGMVWQNRDVYLQGPIWAKIPHTDGNFHPSPLMGGFGLKHPPPQIL |
| | IKNTPVPADPPTTFSQAKLASFITQYSTGQVSVEIEWELQKENSKRWNPE |
| | IQYTSNYYKSTNVDFAVNTEGTYSEPRPIGTRYLTRNL |
| SEQ ID | MAADGYLPDWLEDNLSEGIREWWDLKPGAPKPKANQQKQDDGRGLVLPGY |
| NO: 90 | KYLGPFNGLDKGEPVNAADAAALEHDKAYDQQLKAGDNPYLRYNHADAEF |
| (AAV | QERLQEDTSFGGNLGRAVFQAKKRVLEPLGLVEEGAKTAPGKKRPVEPSP |
| hu.39 | QRSPDSSTGIGKKGQQPARKRLNFGRTGDSESVPDPQPIGEPPAAPSSVG |
| (AAS99286)) | SGTMAAGGGAPMADNNEGADGVGSSSGNWHCDSTWLGDRVITTSTRTWAL |
| | PTYNNHLYKQISNGTSGGSTNDNTYFGYSTPWGYLDENRFHCHFSPRDWQ |
| | RLINNNWGFRPKRLSFKLFNIQVKEVTQNEGTKTIANNLASTIQVFTDSE |
| | YQPPYVLGSAHQGCLPPFPADVFMIPQYGYLTLNNGSQAVGRSSFYCLEY |
| | FPSQMLRTGNNFSFSYTFEDVPFHSSYAHSQSLDRLMNPLIDQYLYYLSR |
| | TQSTGGTAGTQQLLFSRAGPSNMSAQARNWLPGPCYRQQRVSTTLSQNNN |
| | SNFAWTGATKYHLNGRDSLVNPGVAMATNKDDEDRFFPSSGILMFGKQGA |
| | GKDNVDYSNVMLTSEEEIKTTNPVATEQYGVVADNLQQQNTAPTVGAVNS |
| | QGALPGMVWQNRDVYLQGPIWAKIPHTDGNFHPSPLMGGFGLKHPPPQIL |
| | IKNTPVPADPPTAFNQAKLNSFIAQYSTGQVSVEIEWELQKENSKRWNPE |
| | IQYTSNYYKSTNADFAVNTEGVYSEPRPIGTRYLTRNL |
| SEQ ID | MAADGYLPDWLEDNLSEGIREWWDLKPGAPKPKANQQKQDDGRGLVLPGY |
| NO: 91 | KYLGPFNGLDKGEPVNAADAAALEHDKAYDQQLKAGDNPYLRYNHADAEF |
| (AAV | QERLQEDTSFGGNLGRAVFQAKKRVLEPLGPVEEAAKTAPGKKRPVEPPP |
| hu.41 | QRSPDSSTGIGKKGQQPAKKRLNFGQTGDSESVPDPQPIGEPPAGPSGLG |
| (AAS99289)) | SGTMAAGGGAPMADNNEGADGVGSSSGNWHCDSTWLGDRVITTSTRTWAL |
| | PTYNNHLYKQISNGTSGGSTNDNTYFGYSTPWGYFDFNRFHCHFSPRDWQ |
| | RLINNNWGFRPKRLSFKLFNIQVKEVTQNEGTKTVANNLTSTIQVFTDSE |
| | YQLPYVLGSAHQGCLPPFPADVFMIPQYGYLTLNNGSQAVGRSSFYCLEY |
| | FPSQMLRTGNNFEFSYTFEDVPFHSSYAHSQSLDRLMNPLIDQYLYYLSR |
| | TQSTGGTQGTQQLLFSQAGPANMSAQAKNWLPGPCYRQQRVSTTLSQNNN |
| | SNFAWTGATKYHLNGRDSLVNPGVAMATHKDDEERFFPSSGVLMFGKQGA |
| | GRDNVDYSSVMLTSEEEIKTTNPVATEQYGVVADNLQQTNTGPIVGNVNS |
| | QGALPGMVWQNRDVYLQGPIWAKIPHTDGNFHPSPLMGGFGLKHPPPQIL |
| | IKNTPVPADPPTTFSQAKLASFITQYSTGQVSVEIEWELQKENSKRWNPE |
| | IQYTSNYYKSTNVDFAVNTEGTYSEPRPIGTRYLTRNL |
| SEQ ID | MAADGYLPDWLEDNLSEGIREWWDLKPGAPKPKANQQKQDDGRGLVLPGY |
| NO: 92 | KYLGPFNGLDKGEPVNAADAAALEHDKAYDQQLKAGDNPYLRYNHADAEF |
| (AAV | QERLQEDTSFGGNLGRAVEQAKKRVLEPLGLVEBAAKTAPGKKRPVEPSP |
| hu.42 | QRSPDSSTGIGKKGQQPAKKRLNFGQTGDSESVPDPQPIGEPPAGPSGLG |
| (AAS99290)) | SGTMAAGGGAPMADNNEGADGVGSSSGNWHCDSTWLGDRVITTSTRTWAL |
| | PTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSE |
| | YQLPYVLGSAHQGCLPPFPADVFMIPQYGYLTLNNGSQAVGRSSFYCLEY |
| | FPSQMLRTGNNFEFSYTFEDVPFHSSYAHSQSLDRLMNPLIDQYLYYLSR |
| | TQSTGGTQGTQQLLFSQAGPANMSAQAKNWLPGPCYRQQRVSTTLSQSNN |
| | SNFAWTGATKYHLNGRDSLVNPGVAMATHKDDEERFFPSSGVLMFGKQGA |
| | GRDNVDYSSVMLTSEEEIKTTNPVATEQYGVVADNLQQTNTGPIVGNVNS |
| | QGALPGMVWQNRDVYLQGPIWAKIPHTDGNFHPSPLMGGLGLKHPPPQIL |
| | IKNTPVPADPPTTFSQAKLASFITQYSTGQVSVEIEWELQKENSKRWNPE |
| | IQYTSNYYKSTNVDFAVNTEGTYSEPRPIGTRYLTRNL |
| SEQ ID | MAADGYLPDWLEDNLSEGIREWWDLKPGAPKPKANQQKQDDGRGLVLPGY |
| NO: 93 | KYLGPFNGLDKGEPVNAADAAALEHDKAYDQQLKAGDNPYPRYNHADAEF |
| (AAV | QERLQEDTPFGGNLGRAVFQAKKRVLEPLGLVEEAAKTAPGKKRPVEPSP |
| hu.43 | QRSPDSSTGIGKKGQQPAKKRLNFGQTGDSESVPDPQPIGEPPAGPSGLG |
| (AAS99291)) | SGTMAAGGGAPMADNNEGADGVGNASGNWHCDSTWLGDRVITTSTRTWAL |
| | PTYNNHLYKQISSASTGASNDNHYFGYSTPWGYFDFNRFHCHFSPRDWQR |
| | LINNNWGFRPKRLNFKLFNIQVKEVTTNDGVTTIANNLTSTVQVFSDSEY |
| | QLPYVLGSAHQGCLPPFPADVFMIPQYGYLTLNNGSQAVGRSSFYCLEYF |
| | PSQMLRTGNNFTFSYTFEEVPLHSSYAHSQSLDRLMNPLIVQYLYYLNRT |
| | QNQSGSAQNKDLLFSRGSPAGMSVQPKNWLPGPCYRQQRVSKTKTDNNNS |
| | NFTWTGASKYNLNGRESIINPGTAMASHKDDEDKFFPMSGVMIFGKESAG |
| | ASNTALDNVMITDEEEIKATNPVATERFGTVAVNFQSSSTDPATGDVHAM |
| | GALPGMVWQDRDVYLQGPIWAKIPHTDGHFHPSPLMGGFGLKNPPPQILI |
| | KNTPVPANPPAEFSATKFASFITQYSTGQVSVEIEWELQKENSKRWNPEV |
| | QYTSNYAKSASVDFTVDNNGLYTEPRPIGTRYLTRPL |
| SEQ ID | MAADGYLPDWLEDTLSEGIRQWWKLRPGPPPPKPAERHKDDSRGLVLPGY |
| NO: 94 | KYLGPFNGLDKGEPVNEADAAALEHDKAYDRQLDSGDNPYLKYNHADAEF |
| (AAV | QERLKEDTSFGGNLGRAVFQAKKRVLEPLGLVEEGAETAPGKKRPVEQSP |
| hu.44 | QGPDSSSGIGKTGQQPAKKRLNFGQTGDSESVPDPQPLGEPPATPAAVGP |
| (AAS99292)) | TTMASGGGAPMADNNEGADGVGNASGNWHCDSTWLGDRVITTSTRTWALP |
| | TYNNHLYKQISSASTGASNDNHYFGYSTPWGYFDFNRFHCHFSPRDWQRL |
| | INNNWGFRPKRLNFKLENIQVKEVTTNDGVTTIANNLTSTVQVFSDSEYQ |
| | LPYVLGSAHQGCLPPFPADVFMIPQYGYLTLNNGSQAVGRSSFYCLEYFP |
| | SQMLRTGNNFTFSYTFEEVPFHSSYAHSQSLDRLMNPLIDQYLYYPNRTQ |
| | NQSGSAQNKDLLFSRGSPAGMSVQPKNWLPGPCYRQQRVSKTKTDNNNSN |
| | FTWTGASKYNLNGRESIINPGTAMASHKDDEDKFFPMSGVMIFGKESAGA |
| | SNTALDNVMITDEEEIKATNPVATERFGTVAVNFQSSSTDPATGDVHAMG |
| | ALPGMVWQGRDVYLQGPIWAKIPHTDGHFHPSPLMGGFGLKNPPPQILIK |
| | NTPVPANPPAEFSATKFASFITQYSTGQVSVEIEWELQKENSKRWNPEVQ |
| | YTSNYAKSANVDFTVDNNGLYTEPRPIGTRYLTRPL |
| SEQ ID | MAADGYLPDWLEDTLSEGIRQWWKLKPGPPPPKPAERHRDDSRGLVLPGY |
| NO: 95 | KYLGPFNGLDKGEPVNEADAAALEHDKAYDRQLDSGDNPYLKYNHADAEF |
| (AAV | QERLKEDTSFGGNLGRAVFQAKKRVLEPLGLVEEPVKTAPGKKRPVEHSP |
| hu.45 | VEPDSSSGTGKAGQQPARKRLNFGQTGDADSVPDPQPLGQPPAAPSGLGT |
| (AAS99293)) | NTMATGSGAPMADNNEGADGVGNSSGNWHCDSTWMGDRVITTSTRTWALP |
| | TYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSGYQL |
| | PYVLGSAHQGCLPPFPADVFMVPQYGYPTLNNGSQAVGRSSFYCLEYFPS |
| | QMLRTGNNFTFSYTFEDVPFHSSYAHSQSLDRLMNPLIDQYLYYLSTTNT |
| | PSGTTTQSRLQFSQAGASDIRDQSRNWLPGPCYRQQRVSKTSADNNNSEY |
| | SWTGATKYHLNGRDSLVNPGPAVASHKDDEEKFFPQSGVLIFGKQGSEKT |
| | NVDIEKVMITDEEEIRTINPVATEQYGSVSTNLQRGNRQAATADVNTQGV |
| | LPGMVWQDRDVYLQGPIWAKIPHTDGHFHPSPLMGGFGLKHPPPQILIKN |
| | TPVPANPSTTFSAAKFASFITQYSTGQVSVEIEWELQKENSKRWNPEIQY |
| | TSNYNKSVNVDFTVDINGVYSEPRPIGTRYLTRNL |
| SEQ ID | MAADGYLPDWLEDTLSEGIRQWWKLKPGPPPPKPAERHKDDSRGLVLPGY |
| NO: 96 | KYLGPFNGLDKGEPVNEADAAALEHDKAYDRQLDSGDNPYLKYNHADAEF |
| (AAV | QERLKEDTSFGGNLGRAVFQAKKRVLEPLGLVEEGAKTAPGKKRPVEQSP |
| hu.46 | QEPDSPSGIGKTGQQPAKKRLNFGQTGDSESVPDPQPLGEPPATPAAVGP |
| (AAS99294)) | TTMASGGGAPMADNNEGADGVGNASGNWHCDSTWLGDRVITTSTRTWALP |
| | TYNNHLYKQISSASTGASNDNHYFGYSTPWGYFDFNRFHCHFSPRDWQRL |
| | INNNWGFRPKRLNFKLENIQVKEVTTNDGVTTIANNLTSTVQVFSDSEYQ |
| | LPYVLGSAHQGRLPPFPADVFMIPQYGYLTLNNGSQAVGRSSSYCLEYFP |
| | SQMLRTGNNFTFSYTFEEVPLHSSCAHSQSLDRLMNPLIDQYLYYLNRTQ |
| | NQSGSAQNRDLLFSRGSPAGMSVQPKNWLPGPCYRQQRVSKTKTDNNNSN |
| | FTWTGASKYNLNGRESIINPGTAMASHKDDEDKFFPMSGVMIFGKESAGA |
| | SNTALDNVMITDEEEIKATNPVATERFGTVAVNFQSSSTDPATGDVHAMG |
| | ALPGMVWQDRDVYLQGPIWAKIPHTDGHFHPSPLMGGFGLKNPPPQILIK |
| | NTPVPANPPAEFSATKFASFITQYSAGQVSVEIEWELQKENSKRWNPEVQ |
| | YTSNYAKSANVDFTVDNNGLYTEPRPIGTRYLTRPL |
| SEQ ID | MAADGYLPDWLEDTLSEGIRQWWKLKPGPPPPKPAERHRDDSRGLVLPGY |
| NO: 97 | KYLGPFNGLDKGEPVNEADAAALEHDKAYDRQLDSGDNPYLKYNHADAEF |
| (AAV | QERLKEDTSFGGNLGRAVFQAKKRVLEPLGLVGEPVKTAPGKKRPVEHSP |
| hu.47 | VEPDSSSGTGKAGQQPARKRLNFGQTGDADSVPDPQPLGQPPAAPSGLGT |
| (AAS99295)) | NTMATGSGAPMADNNEGADGVGNSSGNWHCDSTWMGDRVITTSTRTWALP |
| | TYNNHLYKQISSQSGASNDSHYFGYSTPWGYFDFNRFHCHFSPRDWQRLI |
| | NNNWGFRPKRLNFKLFNIQVKEVTQNDGTTTIANNLTSTVQVFTDSEYQL |
| | PYVLGSAHQGCLPPFPADVFMVPQYGYLTLNNGSQAVGRSSFYCLEYFPS |
| | QMLRTGNNFTFSYTFEDVPFHSSYAHSQSLDRLMNPLIDQYLYYLSTTNT |
| | PSGTTTQSRLQFSQAGASDIRDQSRNWLPGPCYRQQRVSKTSADNNNSEY |
| | SWTGATKYHLNGRDSLVNPGPAMASHKDNEEKFFPQSGVLIFGKQGSEKT |
| | NVDIEKVMITDEEEIRTTNPVATEQYGSVSTNLQRGNRQAATADVNTQGV |
| | LPGMVWQDRDVYLQGPIWAKIPHTDGHFHPSPLMGGFGLKHPPPQILIKN |
| | TPVPANPSTTFSAAKFASFITQYSTGQVSVEIEWELQKENSKRWNPEIQY |
| | TSNYNKSVNVDFTVDINGVYSEPRPIGTRYLTRNL |
| SEQ ID | MAADGYLPDWLEDNLSEGIREWWDLKPGAPKPKANQQKQDDGRGLVLPGY |
| NO: 98 | KYLGPFNGLDKGEPVNAADAAALEHDKAYDQQLKAGDNPYLRYNHADAEF |
| (AAV | QERLQEDTSFGGNLGRAVFQAKKRVLEPLGLVEEGAKTAPGKKRPVEQSP |
| hu.48 | QEPDSSSGIGKTGQQPAKKRLNFGQTGDSESVPDPQPLGEPPATPAAVGP |
| (AAS99296)) | TTMASGGGAPMADNNEGADGVGNASGNWHCDSTWLGDRVITTSTRTWALP |
| | TYNNHLYKQISSTSTGASNDNHYFGYGTPWGYFDFNRFHCHFSPRDWQRL |
| | INNNWGFRPKRLNEKLFNIQVEEVTTNDGVTTIANNLTSTVQVFSDSEYQ |
| | LPYVLGSAHQGCLPPFPADVFMIPQYGYLTLNNGSQAVGRSSFYCLEYFP |
| | SQMLRTGNNFTFSYTFEEVPFHSSYAHSQSLDRLMNPLIDQYLYYLNRTQ |
| | NQSGSAQNKDLLFSRGSPAGMSVQPKNWLPGPCYRQQRVSKTKTDNNNSN |
| | FTWTGASKYNLNGRESIINPGTAVASHKDDEDKFFPMSGVMIFGKESAGA |
| | SSTALDNVMITDEEEIKATNPVATERFGTVAVNFQSSSTDPATGDVHAMG |
| | ALPGMVWQDRDVYLQGPIWAKIPHTDGHFHPSPLMGGFGLKNPPPQILIK |
| | NTPVPANPPAEFSATKFASFITQYSTGQVSVEIEWELQKENSKRWNPEVQ |
| | YTSNYAKSANVDFTVDNNGLYTEPRPIGTRYLTRPL |
| SEQ ID | MAADGYLPDWLEDTLSEGIRQWWKLKPGPPPPKPAERHKDDSRGLVLPGY |
| NO: 99 | KYLGPFNGLDKGEPVNEADAAALEHDKAYDRQLDSGDNPYLKYNHADAEF |
| (AAV | QERLKEDTSFGGNLGRAVFQAKKRVLEPLGLVGEPVKTAPGKKRPVEHSP |
| hu.51 | VEPDSSSGTGKAGQQPARKRLNFGQTGDADSVPDPQPLGQPPAAPSGLGT |
| (AAS99298)) | NTMATGSGAPMADNNEGADGVGNSSGNWHCDSTWMGDRVITTSTRTWALP |
| | TYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQL |
| | PYVLGSAHQGCLPPFPADVFMVPQYGYLTLNNGSQAVGRSSFYCLEYFPS |
| | QMLRTGNNFTFSYTFEDVPFHSGYAHSQSLDRLMNPLIDQYLYYLSTTNT |
| | PSGTTTQSRLQFSQAGASDIRDQSRNWLPGPCYRQQRVSKTSADNNNSEY |
| | SWTGATKYHLNGRDSLVNPGPAMASHKDNEEKFFPQSGVLIFGKQGSEKT |
| | NVDIEKVMITDEEEIRTTNPVATEQYGSVSTNLQRGNRQAATADVNTQGV |
| | LPGMVWQDRDVYLQGPIWAKIPHTDGHFHPSPLMGGFGLKHPPPQILIKN |
| | TPVPANPSTTFSAAKFASFITQYSTGQVSVEIEWELQKENSKRWNPEIQY |
| | TSNYNKSVNVDFTVDINGVYSEPRPIGTRYLTRNL |
| SEQ ID | MAADGYLPDWLEDTLSEGIRQWWKLKPGPPPPKPAERHKDDSRGLVLPGY |
| NO: | KYLGPFNGLDKGEPVNEADAAALEHDKAYDRQLDSGDNPYLKYNHADAEF |
| 100 | QERLKEDTSFGGNLGRAVFQAKKRVLEPLGLVGEPVKTAPGKKRPVEHSP |
| (AAV | VEPDSSSGTGKAGQQPARKRLNFGQTGDADSVPDPQPLGQPPAAPSGLGT |
| hu.52 | NTMATGSGAPMADNNEGADGVGNSSGNRHCDSTWMGDRVITTSTRTWALP |
| (AAS99299)) | TYNNHLYRQISSQSGASNDNHYFGYSTPWGYFDFNRFHCHFSPRDWQRLI |
| | NNNWGFRPKRLNFKLENIQVKEVTQNDGTTTIANNLTSTVQVFTDSEYQL |
| | PYVLGSAHQGCLPPFPADVFMVPQYGYLTLSNGSQAVGRSSFYCPEYFPS |
| | QMLRTGNNFTFSYTFEDVPFHSSYAHSQSLDRLMNPLIDQYLYYLSTTNT |
| | PSGTTTQSRLQFSQAGASDIRDQSRNWLPGPCYRQQRVSKTSADNNNSEY |
| | SWTGATKYHLNGRDSLVNPGPAMASHKDNEEKFFPQSGVLIFGKQGSEKT |
| | NVDIEKVMITDEEEIRTTNPVATEQYGSVSTNLQRGNRQAATADVNTQGV |
| | LPGMVWQDRDVYLQGPIWAKIPHTDGHFHPSPLMGGFGPKHPPPQILIKN |
| | TPVPANPSTTFSAAKFASFITQYSTGQVSVEIEWELQKENSKRWNPEIQY |
| | TSNYNKSVNVDFTVDINGVYSEPRPIGTRYLTRNL |
| SEQ ID | MAADGYLPDWLEDTLSEGIRQWWKLKPGPPPPKPAERHKDDSRGLVLPGY |
| NO: | KYLGPFNGLDKGEPVNEADAAALEHDKAYDRQLDSGDNPYLKYNHADAEF |
| 101 | QERLKEDTSFGGNLGRAVFQAKKRVLEPLGLVEEPVKTAPGKKRPVEHSP |
| (AAV | AEPDSSSGTGKAGQQPARKRLNFGQTGDADSVPDPQPLRQPPAAPTSLGS |
| hu.53 | TTMATGSGAPMADNNEGADGVGNSSGNWHCDSQWLGDRVITTSTRTWALP |
| (AAS99300)) | TYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQL |
| | PYVLGSAHQGCLPPFPADVFMVPQYGYLTLNNGSQAVGRSSFYCLEYFPS |
| | QMLRTGNNFQFSYTFEDVPFHSSYAHSQSLDRLMNPLIDQYLYYLNRTQT |
| | ASGTQQSRLLFSQAGPTSMSLQAKNWLPGPCYRQQRLSKQANDNNNSNFP |
| | WTGATKYYLNGRDSLVNPGPAMASHKDDEEKFFPMHGTLIFGKEGTNATN |
| | AELENVMITDEEEIRTTNPVATEQYGYVSNNLQNSNTAASTETVNHQGAL |
| | PGMVWQDRDVYLQGPIWAKIPHTDGHFHPSPLMGGFGLKHPPPQIMIKNT |
| | PVPANPPTNFSSAKFASFITQYSTGQVSVEIEWELQKENSKRWNPEIQYT |
| | SNYNKSVNVDFTVDINGVYSEPRPIGTRYLTRNL |
| SEQ ID | MAADGYLPDWLEDTLSEGIRQWWKLKPGPPPPKPAERHKDDSRGLVLPGY |
| NO: | KYLGPFNGLDKGEPVNEADAAALEHDKAYDRQLDSGDNPYLKYNHADAEF |
| 102 | QERLKEDTSFGGNLGRAVFQAKKRVLEPLGLVEEPVKTAPGKKRPVEHSP |
| (AAV | AEPDSSSGTGKAGQQPARKRLNFGQTGDADSVPDPQPLGQPPAAPTSLGS |
| hu.54 | TTMATGSGAPMADNNEGADGVGNSSGNWHCDSQWLGDRVITTSTRTWALP |
| (AAS99301)) | TYNNHLYKQISSQSGASNDNHYFGYSTPWGYFDFNRFHCRFSPRDWQRLI |
| | NNNWGFRPKRLNFKLFNIQVKEVTQNDGTTTIANNLTSTVQVFTDSEYQL |
| | PYVLGSAHQGCLPPFPADVFMVPQYGYLTLNNGSQAVGRSSFYCLEYFPS |
| | QMLRTGNNFQFSYTFEDVPFHSSYAHSQGLDRLMNPLIDQYLYYLNRTQT |
| | ASGTQQSRLLFSQAGPTSMSLQAKNWLPGPCYRQQRLSKQANDNNNSNFP |
| | WTGATKYHLNGGDSLVNPGPAMASHKDDEEKFFPMHGTLIFGKEGTNATN |
| | AELENVMITDEEEIRTTNPVATEQYGYVSNNLQNSNTAASTETVNHQGAL |
| | PGMVWQDRDVYLRGPIWAKIPHADGHFHPSPLMGGFGLKHPPPQIMIKNT |
| | PVPANPPTNFSSAKFASFITQYSTGQVSVEIEWELQKENSKRWNPEIQYT |
| | SNYNKSVNVDFTVDINGVYSEPRPIGTRYLTRNL |
| SEQ ID | MAADGYLPDWLEDTLSEGIRQWWKLKPGPPPPKPAERHKDDSRGLVLPGY |
| NO: | KYLGPFNGLDKGEPVNEADAAALEHDKAYDRQLDSGDNPYLKYNHADAEF |
| 103 | QERLKEDTSFGGNLGRAVFQAKKRVLEPLGLVEEPVKTAPGKKRPVEHSP |
| (AAV | AEPDSSSGTGKAGQQPARKRLNFGQTGDADSVPDPQPLGQPPAAPTSLGS |
| hu.55 | TTMATGSGAPMADNNEGADGVGNSSGNWHCDSQWLGDRVITTSTRTWALP |
| (AAS99302)) | TYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQL |
| | PYVLGSAHQGCLPPFPADVFMVPQYGYLTLNNGSQAVGRSSFYCLECFPS |
| | QMLRTGNNFQFSYTFEDVPFHSSYAHSQSLDRLMNPLIDQYLYYLNRTQT |
| | ASGTQQSRLLFSQAGPTSMSLQAKNWLPGPCYRQQRLSKQANDNNNSNFP |
| | WTGATKYHLNGRDSLVNPGPAMASHKDDEEKFFPMHGTLIFGKEGTNATN |
| | AELENVMITDEEEIRTTNPVATEQYGYVSNNLQNSNTAASTETVNHQGAL |
| | PGMVWQDRDVYLQGPIWAKIPHTDGHFHPSPLMGGFGLKHPPPQIMIKNT |
| | PVPANPPTNFSSAKFASFITQYSTGQVSVEIEWELQKENSKRWNPEIQYT |
| | SNYNKSVNVDFTVDINGVYSEPRPIGTRYLTRNL |
| SEQ ID | MAADGYLPDWLEDTLSEGIRQWWKLKPGPPPPKPAERHKDDSRGLVLPGY |
| NO: | KYLGPFNGLDKGEPVNEADAAALEHDKAYDRQLDSGDNPYLKYNHADAEF |
| 104 | QERLKEDTSFGGNLGRAVFQAKKRVLEPLGLVEEPVKTAPGKKRPVEHSP |
| (AAV | VEPDSSSGTGKAGNQPARKRLNFGQTGDADSVPDPQPLGQPPASPSGLGT |
| hu.56 | NTMATGSGAPMADNNEGADGVGNSSGNWHCDSTWMGDRVVTTSTRTWALP |
| (AAS99303)) | TYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDLEYQL |
| | PYVLGSAHQGCLPPFPADVFMVPQYGYLTLNNGSQAVGRSSFYCLEYFPS |
| | QMLRTGNNFTFSYTFEDVPFHSSYAHSQSLDRLMNPLIDQYLYYLSRTNT |
| | PSGTTTQSRLQFSQAGASDIRDQSRNWLPGPCYRQQRVSKTAADNNNSEY |
| | SWTGATKYHLNGRDSLVNPGPAMASHKDDEEKFFPQSGVLIFGKQGSEKT |
| | NVDIEKVMITDEEEIRTTNPVATEQYGSVSTNLQSGNTQAATSDVNTQGV |
| | LPGMVWQDRDVYLQGPIWAKIPHTDGHFHPSPLMGGFGLKHPPPQILIKN |
| | TPVPANPSTTFSAAKFASFITQYSTGQVSVEIEWELQKENSKRWNPEIQY |
| | TSNYNKSVNVDFTVDTNGVYSEPRPIGTRYLTRNL |
| SEQ ID | MAADGYLPDWLEDTLSEGIRQWWKLKPGPPPKPAERHKDDSRGLVLPGYK |
| NO: | YLGPFNGLDKGEPVNEADAAALEHDKAYDRQLDSGDNPYLKYNHADAEFQ |
| 105 | ERLKEDTSFGGNLGRAVFQAKKRVLEPLGLVEEPVKTAPGKKRPVEHSPV |
| (AAV | EPDSSSGTGKAGNQPARKRLNFGQTGDADSVPDPQPLGQPPAAPSGLGTN |
| hu.57 | TMATGSGAPMADNNEGADGVGNSSGDWHCDSTWMGDRVITTSTRTWALPT |
| (AAS99304)) | YNNHLYKQISSQSGASNDNHYFGYSTPWGYFDFNRFHCHFSPRDWQRLIN |
| | NNWGFRPKRLNLKLFNIQVKEVTQNDGTTTIANNLTSTVQVFTDLEYQLP |
| | YVLGSAHQGCLPPFPADVFMVPQYGYLTLNNGSQAVGRSSFYCLEYFPSQ |
| | MLRTGNNFTFSYTFEDVPFHSSYAHSQSLDRLMNPLIDQYLYYLSRTNTP |
| | SGTTTQSRLQFSQAGASDIRDQSRNWLPGPCYRQQRVSKTAADNNNGEYS |
| | WTGATKYHLNGRDSLVNPGPAMASHKDDEEKFFPQSGVLIFGKQGSEKTN |
| | VDIEKVMITDEEEIRTTNPVATEQYGSVSTNLQSGNTRAATSDVNTQGVL |
| | PGMVWQDRDVYLQGPIWAKIPHTDGHFHPSPLMGGFGLKHPPPQILIKNT |
| | PVPANPSTTFSAAKFASFITQYSTGQVSVEIEWELQKENSKRWNPEIQYT |
| | SNYNKSVNVDFTVDINGVYSEPRPIGTRYLTRNL |
| SEQ ID | MAADGYLPDWLEDTLSEGIRQWWKLKPGPPPPKPAERHKDDSRGLVLPGY |
| NO: | KYLGPFNGLDKGEPVNEADAAALEHDKAYDRQLDSGDNPYLKYNHADAEF |
| 106 | QERLKEDTSFGGNLGRAVFQAKKRVLEPLGLVEEPVKTAPGKKRPVEHSP |
| (AAV | VEPDSSSGTGKAGQQPARKRLNFGQTGDADSVPDPQPLGQPPAAPSGLGS |
| hu.60 | TTMATGSGAPMADNNEGADGVGNSSGNWHCDSQWLGDRVITTSTRTWALP |
| (AAS99307)) | TYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQL |
| | PYVLGSAHQGCLPPFPADVFMVPQYGYLTLNNGSQAVGRSSFYCLEYFPS |
| | QMLRTGNNFQFSYTFEDVPFHSSYAHSQSLDRLMNPLVDQYLYYLNKTQT |
| | NSGTLQQSRLLFSQAGPTNMSLQAKNWLPGPCYRQQRLSKQANDNNNSNF |
| | PWTAATKYHLNGRDSLVNPGPAMASHKDDEEKFFPMHGTLIFGKQGTNAN |
| | DADLENVMITDEEEIRTTNPVATEQYGTVSNNLQNSNTGPTTGTVNHQGA |
| | LPGMVWQDRDVYLQGPIWAKIPHTDGHFHPSPLMGGFGLKHPPPQIMIKN |
| | TPVPANPPTNFSSAKFASFITQYSTGQVSVEIEWELQKENSKRWNPEIQY |
| | TSNYNKSVNVDFTVDINGVYSEPRPIGTRYLTRNL |
| SEQ ID | MAADGYLPDWLEDTLSEGIRQWWKLKPGPPPPKPAERHKDDSRGLVLPGY |
| NO: | KYLGPFNGLDKGEPVNEADAAALEHDKAYDRQLDSGDNPYLKYNHADAEF |
| 107 | QERLKEDTSFGGNLGRAVFQAKKRVLEPLGLVEEPVKTAPGKKRPVEHPP |
| (AAV | VEPDSSSGTGKAGQQPARKRLNFGQTGDADSVPDPQPLGQPPAAPSGLGS |
| hu.61 | TTMATGSGAPMADNNEGADGVGNSSGNWHCDSQWLGDRVITTSTRTWALP |
| (AAS99308)) | TYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQL |
| | PYVLGSAHQGCLPPFPADVFMVPQYGYLTLNNGSQAVGRSSFYCLEYFPS |
| | QMLRTGNNFQFSYTFEDVPFHSSYAHSQSLDRLMNPLIDQYLYYLNKTQT |
| | NSGTLQQSRLLFSQAGPTNMSLQAKNRLPGPCYRQQRLSKQANDNNNSNF |
| | PWTAATKYHLNGRDSLVNPGPAMASHKDDEEKFFPMHGTLIFGKQGTNAN |
| | DADLENVMITDEEEIRTTNPVATEQYGTVSNNLQNSNTGPTTGTVNHQGA |
| | LPGMVWQDRDVYLQGPIWAKIPHTDGHFHPSPLVGGFGLKHPPPQIMIKN |
| | TPVPANPPTNFSSAKFASFITQYSTGQVSVEIEWELQKENSKRWNPEIQY |
| | TSNYNKSVNVDFTVDINGVYSEPRPIGTRYLTRNL |
| SEQ ID | MAADGYLPDWLEDTLSEGIRQWWKLKPGPPPPKPAERHKDDSRGLVLPGY |
| NO: | KYLGPFNGLDKGEPVNEADAAALEHDKAYDRQLDSGDNPYPKYNHADAEF |
| 108 | QERLKEDTSFGGNLGRAVFQAKKRVLEPLGLVEEPVKTAPGKKRPVEHSP |
| (AAV | AEPDSSSGTGKAGQQPARKRLNFGQTGDADSVPDPQPLGQPPAAPSGLGT |
| hu.63 | NTMATGSGAPMADNNEGADGVGNSSGNWHCDSTWMGDRVITTSTRTWALP |
| (AAS99309)) | TYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQL |
| | PYVLGSAHQGCLPPFPADVFMVPQYGYLTLNNGSQAVGRSSFYCLEYFPS |
| | QMLRTGNNFTFSYTFEDVPFHSSYAHSQSLDRLMNPLIDQYLYYLSRTNT |
| | PSGTTTQSRLQFSQAGASDIRDQSRNWLPGPCYRQQRVSKTSADNNNSEY |
| | SWTGATKYHLNGRDSLVNPGPAMASHKDDEEKFFPQSGVLIFGKQDSGKT |
| | NVDIEKVMITDEEEIRTTNPVATEQYGSVSTNLQSGNTQAATSDVNTQGV |
| | LPGMVWQDRDVYLQGPIWAKIPHTDGHFHPSPLMGGFGLKHPPPQILIKN |
| | TPVPANPSTTFSAAKFASFITQYSTGQVSVEIEWELQKENSKRWNPEIQY |
| | TSNYNKSVNVDFTVDTNGVYSEPRPIGTRYLTRNL |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLKAGDNPYLRYNHADAEF |
| 109 | QERLQEDTSFGGNLGRAVFQAKKRVLEPLGLVEEAAKTAPGKKRPVEPSP |
| (AAV | QRSPDSSAGIGKKGQQPAKKRLNFGQTGDSESVPDPQPIGEPPAGPSGLG |
| hu.66 | SGTMAAGGGAPMADNNEGADGVGSSSGNWHCDSTWLGDRVITTSTRTWAL |
| (AAS99311)) | PTYNNHLYKQISNGTSGGSTNDNTYFGYSTPWGYFDFNRFHCHFSPRDWQ |
| | RLINNNWGFRPKRLSFKLFNIQVKEVTQNEGTETIANNLTSTIQVFTDSE |
| | YQLPYVLGSAHQGCLPPFPADVFMIPQYGYLTLNNGSQAVGRSSFYCLEY |
| | FPSQMLRTGNNFEFSYTFEDVPFHSSCAHSQSSDRLMNPLIDQYLYYLSR |
| | TRSTGGTQGTQQLLFSQAGPANMSAQAKNWLPGPCYRQQRVSTTLSQNNN |
| | SNFAWTGATKYHLNGRDSLVNPGVAMATHKDDEERFFPSSGVLMFGKQGA |
| | GRDNVDYSSVMLTSEEEIKTTNPVATEQYGVVADNLQQTNTGPIVGNVNS |
| | QGALPGMVWQNRDVYLQGPIWAKIPHTDGNFHPSPLMGGFGLKHPPPQIL |
| | IKNTPVPADPPTTFSQAKLASFITQYSTGQVSVEIEWELQKENSKRWNPE |
| | IQYTSNYYKSTNVDFAVNTEGTYSEPRPIGTRYLTRNL |
| SEQ ID | MAADGYLPDWLEDNLSEGIREWWDLKPGAPKPKANQQKQDDGRGLVLLGY |
| NO: | KYLGPFNGLDKGEPVNAADAAALEHDKAYDQQLKAGDNPYLRYNHADAEF |
| 110 | QERLQEDTSFGGNLGRAVFQAKKRVLEPLGLVEBAAKTAPGKKRPVEPSP |
| (AAV | QRSPDSSTGIGKKGQQPAKKRLNFGQTGDSESVPDPQPIGEPPAGPSGLG |
| hu.67 | SGTMAAGGGAPMADNNEGADGVGSSSGNWHCDSTWLGDRVITTSTRTWAL |
| (AAS99312)) | PTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSE |
| | YQLPYVLGSAHQGCLPPFPADVFMIPQYGYLTLNNGSQAVGRSSFYCLEY |
| | FPSQMLRTGNNFEFSYTFEDVPFHSGYAHSQSLDRLMNPLIDQYLYYLSR |
| | TQSTGGTQGTQQLLFSQAGPANMSAQAKNWLPGPCYRQQRVSTTLSQNNN |
| | SNFAWTGATKYHLNGRDSLVNPGVAMATHKDDEERFFPSSGVLMFGKQGA |
| | GRDNVDYSSVMLTSEEEIKTTNPVATEQYGVVADNLQQTNTGPIVGNVNS |
| | QGALPGMVWQNRDVYLQGPIWAKIPHTDGNFHPSPLMGGFGLKHPPPQIL |
| | IKNTPVPADPPTTFSQAKLASFITQYSTGQVSVEIEWELQKENSKRWNPE |
| | IQYTSNYYKSTNVDFAVNTEGTYSEPRPIGTRYLTRNL |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLKAGDNPYLRYNHADAEF |
| 111 | QERLQEDTSFGGNLGRAVFQAKKRVLEPLGLVEEGAKTAPGKKRPVEPSP |
| (AAV | QRSPDSSTGIGKKGQQPAKKRLNFGQTGDSESVPDPQPIGEPPAGPSGLG |
| rh.10 | SGTMAAGGGAPMADNNEGADGVGSSSGNWHCDSTWLGDRVITTSTRTWAL |
| (AAO88201)) | PTYNNHLYKQISNGTSGGSTNDNTYFGYSTPWGYFDFNRFHCHFSPRDWQ |
| | RLINNNWGFRPKRLNFKLFNIQVKEVTQNEGTKTIANNLTSTIQVFTDSE |
| | YQLPYVLGSAHQGCLPPFPADVFMIPQYGYLTLNNGSQAVGRSSFYCLEY |
| | FPSQMLRTGNNFEFSYQFEDVPFHSSYAHSQSLDRLMNPLIDQYLYYLSR |
| | TQSTGGTAGTQQLLFSQAGPNNMSAQAKNWLPGPCYRQQRVSTTLSQNNN |
| | SNFAWTGATKYHLNGRDSLVNPGVAMATHKDDEERFFPSSGVLMFGKQGA |
| | GKDNVDYSSVMLTSEEEIKTTNPVATEQYGVVADNLQQQNAAPIVGAVNS |
| | QGALPGMVWQNRDVYLQGPIWAKIPHTDGNFHPSPLMGGFGLKHPPPQIL |
| | IKNTPVPADPPTTFSQAKLASFITQYSTGQVSVEIEWELQKENSKRWNPE |
| | IQYTSNYYKSTNVDFAVNTDGTYSEPRPIGTRYLTRNL |
| SEQ ID | MAADGYLPDWLEDNLSEGIREWWDLKPGAPKPKANQQKQDDGRGLVLPGY |
| NO: | KYLGPFNGLDKGEPVNEADAAALEHDKAYDKQLEQGDNPYLKYNHADAEF |
| 112 | QERLQEDTSFGGNLGRAVFQAKKRVLEPLGLVEEGAKTAPGKKRPIESPD |
| (AAV | SSTGIGKKGQQPAKKKLNFGQTGDSESVPDPQPLGEPPAAPSGLGSGTMA |
| rh.13 | AGGGAPMADNNEGADGVGNASGNWHCDSTWLGDRVITTSTRTWALPTYNN |
| (AAO88199)) | HLYKQISSQSGATNDNHFFGYSTPWGYFDFNRFHCHFSPRDWQRLINNNW |
| | GFRPRKLRFKLFNIQVKEVTTNDGVTTIANNLTSTIQVFSDSEYQLPYVL |
| | GSAHQGCLPPFPADVEMIPQYGYLTLNNGSQSVGRSSFYCLEYFPSQMLR |
| | TGNNFEFSYTFEEVPFHSSYAHSQSLDRLMNPLIDQYLYYLARTQSTTGS |
| | TRELQFHQAGPNTMAEQSKNWLPGPCYRQQRLSKNIDSNNNSNFAWTGAT |
| | KYHLNGRNSLTNPGVAMATNKDDEDQFFPINGVLVFGETGAANKTTLENV |
| | LMTSEEEIKTTNPVATEEYGVVSSNLQSSTAGPQTQTVNSQGALPGMVWQ |
| | NRDVYLQGPIWAKIPHTDGNFHPSPLMGGFGLKHPPPQILIKNTPVPANP |
| | PEVFTPAKFASFITQYSTGQVSVEIEWELQKENSKRWNPEIQYTSNYAKS |
| | NNVEFAVNNEGVYTEPRPIGTRYLTRNL |
| SEQ ID | MAADGYLPDWLEDNLSEGIREWWDLKPGAPKPKANQQKQDDGRGLVLPGY |
| NO: | KYLGPFNGLDKGEPVNEADAAALEHDKAYDKQLEQGDNPYLKYNHADAEF |
| 113 | QERLQEDTSFGGNLGRAVFQAKKRVLEPLGLVEEGAKTAPGKKRPVEPSP |
| (AAV | QRSPDSSTGIGKTGQQPAKKRLNFGQTGDSESVPDPQPIGEPPAGPSGLG |
| rh.19 | SGTMAAGGGAPMADNNEGADGVGSSSGNWHCDSTWLGDRVITTSTRTWAL |
| (AAO88194)) | PTYNNHLYKQISNGTSGGSTNDNTYFGYSTPWGYFDFNRFHCHFSPRDWQ |
| | RLINNNWGFRPRKLRFKLFNIQVKEVTTDDGVTTIANNLTSTIQVFSDSE |
| | YQLPYVLGSAHQGCLPPFPADVFMIPQYGYLTLNNGSQSVGRSSFYCLEY |
| | FPSQMLRTGNNFEFSYTFEEVPFHSSYAHSQSLDRLMNPLIDQYLYYLAR |
| | TQSTTGSTRELQFHQAGPNTMAEQSKNWLPGPCYRQQRLSKNIDSNNNSN |
| | FAWTGATKYHLNGRNSLTNPGVAMATNKDDEDQFFPINGVLVFGKTGAAN |
| | KTTLENVLMTSEEEIKTTNPVATEEYGVVSSNLQSSTAGPQTQTVNSQGA |
| | LPGMVWQNRDVYLQGPIWAKIPHTDGNFHPSPLMDGFGLKHPPPQILIKN |
| | TPVPANPPEVFTPAKFASFITQYSTGQVSVEIEWELQKENSKRWNPEIQY |
| | TSNYAKSNNVEFAVNNEGVYTEPRPIGTRYLTRNL |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLKAGDNPYLRYNHADAEF |
| 114 | QERLQEDTSFGGNLGRAVFQAKKRVLEPLGLVEEGAKTAPGKKRPIESPD |
| (AAV | SSTGIGKKGQQPAKKKLNFGQTGDSESVPDPQPIGEPPAGPSGLGSGTMA |
| rh.22 | AGGGAPMADNNEGADGVGNASGNWHCDSTWLGDRVITTSTRTWALPTYNN |
| (AAO88192)) | HLYKQISSQSGATNDNHFFGYSTPWGYFDFNRFHCHFSPRDWQRLINNNW |
| | GFRPRKLRFKLFNIQVKEVTTNDGVTTIANNLTSTIQVESDSEYQLPYVL |
| | GSAHQGCLPPFPADVFMIPQYGYLTLNNGSQSVGRSSFYCLEYFPSQMLR |
| | TGNNFEFSYTFEEVPFHSSYAHSQSLDRLMNPLIDQYLYYLARTQSTTGS |
| | TRELQFHQAGPNTMAEQSKNWLPGPCYRRQRLSKDIDSNNNSNFAWTGAT |
| | KYHLNGRNSLTNPGVAMATNKDDEDQFFPINGVLVFGKTGAANKTTLENV |
| | LMTSEEEIKTTNPVATEEYGVVSSNLQSSTAGPQTQTVNSQGALPGMVWQ |
| | NRDVYLQGPIWAKIPHTDGNFHPSPLMGGFGLKHPPPQILIKNTPVPANP |
| | PEVFTPAKFASFITQYSTGQVSVEIEWELQKENSKRWNPEIQYTSNYAKS |
| | NNVEFAVNNEGVYTEPRPIGTRYLTRNL |
| SEQ ID | MAADGYLPDWLEDNLSEGIREWWDLKPGAPKPKANQQKQDDGRGLVLPGY |
| NO: | KYLGPFNGLDKGEPVNEADAAALEHDKAYDKQLEQGDNPYLKYNHADAEF |
| 115 | QERLQEDTSFGGNLGRAVFQAKKRVLEPLGLVEEGAKTAPGKKRPVEPSP |
| (AAV | QRSPDSSTGIGKTGQQPAKKRLNFGQTGDSESVPDPQPIGEPPAGPSGLG |
| rh.23 | SGTMAAGGGAPMADNNEGADGVGSSSGNWHCDSTWLGDRVITTSTRTWAL |
| (AAO88191)) | PTYNNHLYKQISNGTSGGSTNDNTYFGYSTPWGYFDFNRFHCHFSPRDWQ |
| | RLINNNWGFRPKRLNFKLFNIQVKEVTQNEGTKTIANNLTSTIQVFTDSE |
| | YQLPYVLGSAHQGCLPPFPADVFMIPQYGYLTLNNGSQAVGRSSFYCLEY |
| | FPSQMLRTGNNFEFSYQFEDVPFHSSYAHSQSLDRLTNPLIDQYLYYLAR |
| | TQSTTGSTRGLQFHQAGPNTMAEQSKNWLPGPCYRQQRLSKNIDSNNNSN |
| | FAWTGATKYHLNGRNSLTNPGVAMATNKDDEDQFFPINGVLVFGKTGAAN |
| | KTTLENVLMTSEEEIKTTNPVATEEYGVVSSNLQSSTAGPQTQTVNSQGA |
| | LPGMVWQNRDVYLQGPIWAKIPHTDGNFHPSPLMGGFGLKHPPPQILIKY |
| | TSNYYKSTNVDFAVNTEGTYSEPRPIGTRYLTRNL |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLKAGDNPYLRYNHADAEF |
| 116 | QERLQEDTSFGGNLGRAVFQAKKRVLEPLGLVEEGAKTAPGKKRPIESPD |
| (AAV | SSTGIGKKGQQPAKKKLNFGQTGDSESVPDPQPIGEPPAGPSGLGSGTMA |
| rh.24 | AGGGAPMADNNEGADGVGSSSGNWHCDSTWLGDRVITTSTRTWALPTYNN |
| (AAO88190)) | HLYKQISNGTSGGSTNDNTYFGYSTPWGYFDFNRFHCHFSPRDWQRLINN |
| | NWGFRPKRLNFKLFNIQVKEVTQNEGTKTIANNLTSTIQVFTDSEYQLPY |
| | VLGSAHQGCLPPFPADVFMIPQYGYLTLNNGSQAVGRSSFYCLEYFPSQM |
| | LRTGNNFEFSYQFEDVPFHSSYAHSQSLDRLMNPLIDQYLYYLSRTQSTG |
| | GTAGTQQLLFSQAGPNNMSAQAKNWLPGPCYRQQRVSTTVSQNNNSNFAW |
| | TGATKYHLNGRDSLVNPGVAMATHKGDEERFFPSSGVLMFGKQGAGKDNV |
| | DYSSVMLTSEEEIKTTNPVATEQYGVVADNLQQQNAAPIVGAVNSQGALP |
| | GMVWQNRDVYLQGPIWAKIPHTDGNFHPSPLMGGFGLKHPPPQILIKNTP |
| | VPADPPTTFSQAKLASFITQYSTGQVSVEIEWELQKENSKRWNPEIQYTS |
| | NYYKSTNVDFAVNTEGTYSEPRPIGTRYLTRSL |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLKAGDNPYLRYNHADAEF |
| 117 | QERLQEDTSFGGNLGRAVFQAKKRVLEPLGLVEEGAKTAPGKKRPIDSPD |
| (AAV | SSTGIGKKGQQPAKKKLNFGQTGDSESVPDPQPLGEPPAAPSSVGSGTMA |
| rh.35 | AGGGAPMADNNEGADGVGNASGNWHCDSTWLGDRVITTSTRTWALPTYNN |
| (AAO88186)) | HLYKQISSSSSGATNDNHYFGYSTPWGYFDFNRFHCHFSPRDWQRLINNN |
| | WGFRPKKLRFKLFNIQVKEVTTNDGVTTIANNLTSTVQVFSDSEYQLPYV |
| | LGSAHQGCLPPFPADVFMIPQYGYLTLNNGSQSVGRSSFYCLEYFPSQML |
| | RTGNNFEFSYSFEDVPFHSSYAHSQSLDRLMNPLIDQYLYYLARTQSTTG |
| | STRELQFHQAGPNTMAEQSKNWLPGPCYRQQGLSKNLDENNNSNFAWTAA |
| | TKYHLNGRNSLTNPGIPMATNKDDEDQFFPINGVLVFGKTGAANKTTLEN |
| | VLMTSEEEIKTTNPVATEBYGVVSSNLQPSTAGPQSQTINSQGALPGMVW |
| | QNRDVYLQGPIWAKIPHTDGNFHPSPLMGGFGLKHPPPQILIKNTPVPAN |
| | PPEVFTPAKFASFITQYSTGQVSVEIEWELQKENSKRWNPEIQYTSNYAK |
| | SNNVEFAVNPDGVYTEPRPIGTRYLPRNL |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLEAGDNPYLRYNHADAEF |
| 118 | QERLQEDTSFGGNLGRAVFQAKKRVLEPLGLVEEGAKTAPGKKRPVEQSP |
| (AAV | QEPDSSSGIGKKGQQPARKRLNFGQTGDSESVPDPQPLGEPPAAPSGVGP |
| rh.43 | NTMAAGGGAPMADNNEGADGVGSSSGNWHCDSTWLGDRVITTSTRTWALP |
| (AAS99245)) | TYNNHLYKQISNGTSGGATNDNTYFGYSTPWGYFDFNRFHCHFSPRDWQR |
| | LINNNWGFRPKRLSFKLFNIQVKEVTQNEGTKTIANNLTSTIQVFTDSEY |
| | QLPYVLGSAHQGCLPPFPADVFMIPQYGYLTLNNGSQAVGRSSFYCLEYF |
| | PSQMLRTGNNFQFTYTFEDVPFHSSYAHSQSLDRLMNPLIDQYLYYLSRT |
| | QTTGGTANTQTLGFSQGGPNTMANQAKNWLPGPCYRQQRVSTTTGQNNNS |
| | NFAWTAGTKYHLNGRNSLANPGIAMATHKDDEERFFPVTGSCFWQQNAAR |
| | DNADYSDVMLTSEEEIKTTNPVATEEYGIVADNLQQQNTAPQIGTVNSQG |
| | ALPGMVWQNRDVYLQGPIWAKIPHTDGNFHPSPLMGGFGLKHPPPQILIK |
| | NTPVPADPPTTFNQSKLNSFITQYSTGQVSVEIEWELQKENSKRWNPEIQ |
| | YTSNYYKSTSVDFAVNTEGVYSEPRPIGTRYLTRNL |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLKAGDNPYLRYNHADAEF |
| 119 | QERLQEDTSFGGNLGRAVFQAKKRVLEPLGLVEBAAKTAPGKKRPVEPSP |
| (AAV | QRSPDSSTGIGKKGQQPARKRLNFGQTGDSESVPDPQPIGEPPAGPSGLG |
| rh.48 | SGTMAAGGGAPMADNNKGADGVGNASGNWHCDSTWLGDRVITTSTRTWAL |
| (AAS99246)) | PTYNNHLYKQISSQSAGSTNDNVYFGYSTPWGYFDFNRFHCHFSPRDWQR |
| | LINSNWGFRPKKLNEKLFNIQVKEVTTNDGVTTIANNLTSTVQVFSDSEY |
| | QLPYVLGSAHQGCLPPFPADVFMIPQYGYLTLNNGSQSVGRSSFYCLEYF |
| | PSQMLRTGNNFTFSYTFEDVPFHSSYAHSQSLDRLMNPLIDQYLYYLART |
| | QSNAGGTAGNRELQFYQGGPTTMAEQAKNWLPGPCFRQQRVSKTLDQNNN |
| | SNFAWTGATKYHLNGRNSLVNPGVAMATHKDDEERFFPSSGVLIFGKTGA |
| | ANKTTLENVLMTNEEEIRPTNPVATEEYGTVSSNLQAANTAAQTQVVNNQ |
| | GALPGMVWQNRDVYLQGPIWAKIPHTDGNFHPSPLMGGFGLKHPPPQILI |
| | KNTPVPANPPEVFTPAKFASFITQYSTGQVSVEIEWELQKENSKRWNPEI |
| | QYTSNFDKQTGVDFAVDSQGVYSEPRPIGTRYLTRNL |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLKAGDNPHLRYNHADAEF |
| 120 | QERLQEDTSFGGNLGRAVFQAKKRVLEPLGLVEEGAKTAPGKKRPVEPSP |
| (AAV | QRSPDSSTGIGKKGQQPARKRLNFGQTGDSESVPDPQLIGEPPAAPSSVG |
| rb.49 | SGTMAAGGGAPMADNNEGADGVGSSSGNWHCDSTWLGDRVITTSTRTWAL |
| (AAS99247)) | PTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSE |
| | YQLPYVLGSAHQGCLPPFPADVFMIPQYGNLTLNNGSQAVGRSSFYCLEY |
| | FPSQMLRTGNNFSFSYTFEDVPFHSSYAHSQSLDRLMNPLIDQYLYYLSR |
| | TQSTGGTAGTQQLLFSQAGPSNMSAQARNWLPGPCYRQQRVSTTLSQNNN |
| | SNFAWTGATKYHLNGRDSLVNPGVAMATNKDDEDRFFPSSGILMFGKQGA |
| | GKDNMGYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQIL |
| | IKNTPVPADPPTAFNQAKLNSFITQYGTGQVSVEIEWELQKENSKRWNPE |
| | IQYTSNYYKSTNVDFAVNTEGVYSEPRPIGTRYLTRNL |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLKAGDNPYLRYNHADAEF |
| 121 | QERLQEDTSFGGNLGRAVFQAKKRVLEPLGLVEEGAKTAPGKKRPVEPSP |
| (AAV | QRSPDSSTGIGKKGQQPAGKRLNFGQTGDSESVPDPQPIGEPPAAPSSVG |
| rh.50 | SGTMAAGGGAPMADNNEGADGVGSSSGNWHCDSTWLGDRVITTSTRTWAL |
| (AAS99248)) | PTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSE |
| | YQLPYVLGSAHQGCLPPFPADVFMIPQYGYLTLNNGSQAVGRSSFYCLEY |
| | FPSQMLRTGNNFSFSYTFEDVPFHSSYAHSQSLDRLMNPLVDQYLYYLSR |
| | TQSTGGTAGTQQLLFSQAGPSNMSAQARNWLPGPCYRQQRVSTTLSQNNN |
| | SNFAWTGATKYHLNGRDSLVNPGVAMATNKDDEDRFFPSSGILMFGKQGA |
| | GKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQIL |
| | IKNTPVPADPPTAFNQAKLNSFITQYSTGQVSVEIEWELQKENSKRWSPE |
| | IQYTSNYYKSTNVDFAVNTEGVYSEPRPIGTRYLTRNL |
| SEQ ID | MVADGYLPDWLEDNLSEGIREWWDLKPGAPKPKANQQKQGDGRGLVLPGY |
| NO: | KYLGPFNGLDKGEPVNAADAAALEHDKAYDQQLKAGDNPYLRYNHADAEL |
| 122 | QERLQEDTSFGGNLGRAVFQAKKRVLEPLGLVEEGAKTAPGKKRPVEPSP |
| (AAV | QRSPDSSTGIGKKGQQPARKRLNFGQTGDSESVPDPQPIGEPPAAPSSVG |
| rh.51 | SGTMAAGGGAPMADNNEGADGVGSSSGNWHCDSTWLGDRVITTSTRTWAL |
| (AAS99249)) | PTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSE |
| | YQLPYVLGSAHQGCQPPFPADVFMIPQYGYLTLNNGSQAVGRSSFYCLEY |
| | FPSQMLRTGNNFSFSYTFEDVPFHSSYAHSQSLDRLMNPLIDQYLYYLSR |
| | TQSTGGTAGTQQLLFSQAGPSNMSAQARNWLPGPCYRQQRVSTTLSQNNN |
| | SNFAWTGATKYHLNGRDSLVNPGVAMATNKDDEDRFFPSSGILMFGKQGA |
| | GKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQIL |
| | IKNTPVPADPPTAFNQAKLNSFITQYSTGQVSVEIEWEPQKENSKRWNPE |
| | IQYTSNYYKSTNVDFAVNTEGVYSEPRPIGTRYLTRNL |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLKAGDNPYLRYNHADAEF |
| 123 | QERLQEDTSFGGNLGRAVFQAKKRVLEPLGLVEEGAKTAPGKKRPVEPSP |
| (AAV | QRSPDSSTGIGKKGQQPARKRLNFGQTGDSESVPDPQPIGEPPAAPSSVG |
| rh.52 | SGTMAAGGGAPMADNNEGADGVGSSSGNWHCDSTWLGDRVITTSTRTWAL |
| (AAS99250) | PTYNNHLYKQISNGTSGGSTNDNTYFGYSTPWGYFDFNRFHCHFSPRDWQ |
| | RLINNNWGFRPKRLSFKLFNIQVKEVTQNEGTKTIANSLTSTIQVFTDSE |
| | YQLPYVLGSAHQGCLPPFPADVFMIPQYGYLTPNNGSQAVGRSSFYCLEY |
| | FPSQMLRTGNNFSFSYTFEDVPFHSSYAHSQSLDRLMNPLIDQYLYYLSR |
| | TQSTGGTAGTQQLLSSQAGPSNMSAQARNWLPGPCYRQQRVSTTLSQNNN |
| | SNFAWTGATKYHLNGRDSLVNPGVAMATNKDDEDRFFPSSGILMFGKQGA |
| | GKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQIL |
| | IKNTPVPADPPTAFNQAKLNSFITQYSTGQVSVEIEWELQKENSKRWNPE |
| | IQYTSNYYKSTNVDFAVNTEGVYSEPRPIGTRYLTRNL |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLKAGDNPYLRYNHADAEF |
| 124 | QERLQEDTSFGGNLGRAVFQAKKRVLEPLGLVEEGAKTAPGKKRPVEPSP |
| (AAV | QRSPDSSTGIGKKGQQPARKRLNFGQTGDSESVPDPQPIGEPPAAPSSVG |
| rh.53 | SGTMAAGGGAPMADNNEGADGVGSSSGNWHCDSTWLGDRVITTSTRTWAL |
| (AAS99251)) | PTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSE |
| | YQLPYVLGSAHQGCLPPFPADVFMIPQYGYLTLNNGSQAVGRSSFYCLEY |
| | FPSQMLRTGNNFSFSYTFEDVPFHSSYVHSQSLDRLMNPLIDQYLYYLSR |
| | TQSTGGTAGTQQLLFSQAGPSNMSAQARNWLPGPCYRQQRVSTTLSQNNN |
| | SNFAWTGATKYHLNGRDSLVNSGVAMATNKDDEDRFFPSSGILMFGKQGA |
| | GKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQIL |
| | IKNTPVPADPPTAFNQAKLNSFITQYSTGQVSVEIEWELQKENSKRWNPE |
| | IQYTSNYYKSTNVDFAVNTEGVYSEPRPIGTRYPTRNL |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLKAGDNPYLRYNHADAEF |
| 125 | QERLQEDTSFGGNLGRAVFQAKKRVLEPLGLVERAAKTAPGKKRPVEPSP |
| (AAV | QRSPDSSTGIGKKGQQPARKRLNFGQTGDSESVPDPQPLGEPPAGPSGLG |
| rh.54 | SGTMAAGGGAPMADNNEGADGVGNASGNWHCDSTWLGDRVITTSTRTWAL |
| (AAS99252)) | PTYNNHLYKQISSQSAGSTNDNVYFGYSTPWGYFDFNRFHCHFSPRDWQR |
| | LINNNWGFRPKKLNFKLFNIQVKEVTTNDGVTTIANNLTSTVQVFSDSEY |
| | QLPYVLGSAHQGCLPPFPADVFMIPQYGYLTLNNGSQSVGRSSFYCLEYF |
| | PSQVLRTGNNFTFSYTFEDVPFHSSYAHSQSLDRLMNPLIDQYLYYLART |
| | QSNPGGTSGNRELQFYQGGPSTMAEQAKNWLPGPCFRQQRVSKTLDQNNN |
| | SNFAWTGATKYHLNGRNSLVNPGVAMATHKDDEDRFFPSSGVLIFGKTGA |
| | TNKTTLENVLMTNEEEIRPTNPVATEEYGIVSSNLQAANTAAQTQVVNNQ |
| | GALPGMVWQNRDVYLQGPIWAKIPHTDGNFHPSPLMGGFGLKHPPPQILI |
| | KNTPVPANPPEVFTPAKFASFITQYSTGQVSVEIEWELQKENSKRWNPEI |
| | QYTSNFDKQTGVDFAVDSQGVYSEPRPIGTRYLTRNL |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLKAGDNPYLRYNHADAEF |
| 126 | QERLQEDTSFGGNLGRAVFQAKKRVLEPLGLVEEGAKTAPGKKRPVEPSP |
| (AAV | QRSPDSSTGIGKKGQQPARKRLNFGQTGDSESVPDPQPIGEPPAAPSSVG |
| rh.55 | SGTMAAGGGAPMADNNEGADGVGSSSGNWHCDSTRLGDRVITTSTRTWAL |
| (AAS99253)) | PTYNNHLYKQISSQSAGSTNDNVYFGYSTPWGYFDFNRFHCHFSPRDWQR |
| | LINNNWGFRPKKLNFKLFNIQVKEVTTNDGVTTIANNLTSTVQVFSDSEY |
| | QLPYVLGSAHQGCLPPFPADVFMIPQYGYLTLNNGSQSVGRSSFYCLEYF |
| | PSQMLRTGNNFTFSYTFEDVPFHSSYAHSQSLDRLMNPLIDQYLYYLART |
| | QSNAGGTAGNRELQFYQGGPTTMAEQAKNWLPGPCFRQRRVSKTLDQNNN |
| | SNFAWTGATKYHLNGRNSLVNPGVAMATHKDDEERFFPSSGVLIFGKTGA |
| | ANKTTLENVLMTNEEEIRPTNPVATEEYGTVSSNLQAANTAAQTQVVNNQ |
| | GALPGMVWQNRDVYLQGPIWAKIPHTDGNFHPSPLMGGFGLKHPPPQILI |
| | KNTPVPANPPEVFTPAKFASFITQYSTGQVSVEIEWELQKENSKRWNPEI |
| | QYTSNFDKQTGVDFAVDSQGVYSEPRPIGTRYLTRNL |
| SEQ ID | MAADGYLPDWLEDNLSEGIREWWALKPGAPKPKANQQKQDDGRGLVLPGY |
| NO: | KYLGPFNGLDKGEPVNAADAAALEHDKAYDQQLQAGDNPYLRYNHADAEF |
| 127 | QERLQEDTSFGGNLGRAVFQAKKRVLEPLGLVEEGAKTAPGKKRPVEPSP |
| (AAV | QRSPDSSTGIGKKGQQPARKRLNFGQTGDSESVPDPQPIGEPPAAPSSVG |
| rb.57 | SGTMAAGGGAPMADNNEGADGVGSSSGNWHCDSTWLGDRVITTSTRTWAL |
| (AAS99254)) | PTYNNHLYKQTSNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSE |
| | YQLPYVLGSAHQGCLPPFPADVFMIPQYGYLTLNNGSQAVGRSSFYCLEY |
| | FPSQMLRTGNNFSFSYTFEDVPFHSSYAHSQSLDRLMNPLIDQYLYYLSR |
| | TQSTGGTAGTQQLLFSQAGPSNMSAQARNWLPGPCYRQQRVSTTLSQNNN |
| | SNFAWTGATKYHLNGRDSLVNPGVAMATNKDDEDRFFPSSGILMFGKQGA |
| | GKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQIL |
| | IKNTPVPADPPTAFNQAKLNSFITQYSTGQVSAEIEWELQKENSKRWNPE |
| | IQYTSNYYKSTNVDFAVNTEGVYSEPRPIGTRYLTRNL |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLKAGDNPYLRYNHADAEF |
| 128 | QERLQEDTSFGGNLGRAVFQAKKRVLEPLGLVEEAAKTAPGKKRPVEPSP |
| (AAV | QRSPDSSTGIGKKGQQPARKRLNFGQTGDSESVPDPQPIGEPPAAPSSVG |
| rh.58 | SGTMAAGGGAPMADNNEGADGVGSSSGNWHCDSTWLGDRVITTSTRTWAL |
| (AAS99255)) | PTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSE |
| | YQLPYVLGSAHQGCLPPFPADVFMIPQYGYLTLNNGSQAVGRSSFYCLEY |
| | FPSQMLRTGNNFSFSYTFEDVPFHSSYAHSQSLDRLMNPLIDQYLYYLSR |
| | TQSTGGTAGTQQLLFSQAGPSNMSAQARNWLPGPCYRQQRVSTTLSQNNN |
| | SNFAWTGATKYHLNGRDSLVNPGVAMATNKDDEDRFFPSSGILMFGKQGA |
| | GKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQIL |
| | IKSTPVPADPPTAFNQAKLNSFITQYSTGQVSVEIEWELQKENSKCWNPE |
| | IQYTSNYYKSTNVDFAVNTEGVYSEPRPIGTRYLTRNL |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLKAGDNPYLRYNHADAEF |
| 129 | QERLQEDTSFGGNLGRAVFQAKKRVLEPLGLAERAAKTAPGKKRPVEPSP |
| (AAV | QRSPDSSTGIGKKGQQPARKRLNFGQTGDSESVPDPQPIGEPPAGPSGLG |
| rh.62 | SGTMAAGGGAPMADNNKGADGVGNASGNWHCDSTWLGDRVITTSTRTWAL |
| (AAS99258)) | PTYNNHLYKQISSQSAGSTNDNVYFGYSTPWGYFDFNRFHCHFSPRDWQR |
| | LINNNWGFRPKKLNFKLFNIQVKEVTTGDGVTTIANNLTSTVQVFSDSEY |
| | QLPYVLGSAHQGCLPPFPADVFMIPQYGYLTLNNDSQSVGRSSFYCLEYF |
| | PSQMLRTGNNFTFSYTFEDVPFHSSYAHSQSLDRLMNPLIDQYLYYLART |
| | QSNAGGTAGNRELQFYQGGPTTMAEQAKNWLPGPCFRQQRVSKTLDQNNN |
| | SNFAWTGATKYHLNGRNSLVNPGVAMATHKDDEERFFPSSGVLIFGKTGA |
| | ANKTTLENVLMTNEEEIRPTNPVATEEYGTVSSNLQAANTAAQTQVVNNQ |
| | GALPGMVWQNRDVYLQGPIWAKIPHTDGNFHPSPLMGGFGLKHPPPQILI |
| | KNTPVPANPPEVFTPAKFASFITQYSTGQVSVEIEWELQKENSKRWNPEI |
| | QYTSNFDKQTGVDFAVDSQGVYSEPRPIGTRYLTRNL |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLKAGDNPYLRYNHADAEF |
| 130 | QERLQEDTSFGGNLGRAVFQAKKRVLEPLGLVEEGAKTAPGKKRPVEPSP |
| (AAV | QRSPDSSTGIGKKGQQPARKRLNFGQTGDSESVPDPQPIGEPPAAPSSVG |
| rh.64 | SGTMAAGGGAPMADNNEGADGVGSSSGNWHCDSTWLGDRVITTSTRTWAL |
| (AAS99259)) | PTYNNHLYKQISNGTSGGSTNDNTYFGYSTPWGYFDFNRFHCHFSPRDWQ |
| | RLINNNWGFRPKRLSFKLFNIQVKEVTQNEGTKTIANNLTSTIQVFTDSE |
| | YQLPYVLGSAHQGCLPPFPADVFMIPQYGYLTLNNGSQAVGRSSFYCLEY |
| | FPSQMLRTGNNFSFSYTFEDVPFHSSYAHSQSLDRLMNPLIDQYLYYLSR |
| | TQSTGGTAGTQQLLFSQAGPSNMSAQARNWLPGPCYRQQRVSTTLSQNNN |
| | SNFAWTGATKYHLNGRDSLVNPGVAMATNKDDEDRFFPSSGILMFGKQGA |
| | GKDNVDYSNVMLTSEEEIKTTNPVATEQYGVVADNLQQQNTAPIVGAVNS |
| | QGALPGMVWQNRDVYLQGPIWAKIPHTDGNFHPSPLMGGFGLKHPPPQIL |
| | IKNTPVPADPPTAFNQAKLNSFITQYSTGQVSVEIVWELQKENSKRRNPE |
| | IQYTSNYYKSTNVDFAVNTEGVYSEPRPIGTRYLTRNL |
| SEQ ID | MAADGYLPDWLEDTLSEGIRQWWKLKPGPPPPKPAERHKDDSRGLVLPGY |
| NO: | KYLGPFNGLDKGEPVNEADAAALEHDKAYDRQLDSGDNPYLKYNHADAEF |
| 131 | QERLKEDTSFGGNLGRAVFQAKKRVLEPLGLVEEPVKTAPGKKRPVEHSP |
| (AAV | AEPDSSSGTGKAGQQPARKRLNFGQTGDADSVPDPQPLGQPPAAPSGLGS |
| rh.56 | TTMATGSGAPMADNNEGADGVGNSSGNWHCDSQWLGDRVITTSTRTWAQP |
| (JA400164)) | TYNNHLYKQISSQSGASNDNHYFGYSTPWGYFDFNRFHCHFSPRDWQRLI |
| | NNNWGFRPKRLNFKLFNIQVKEVTQNDGTTTIANNLTSTVQVFTDSEYQL |
| | PYVLGSAHQGCLPPFPADVFMVPQYGYLTLNNGSQAVGRSSFYCLEYFPS |
| | QMLRTGNNFQFSYTFEDVPFHSSYAHSQSLDRLMNPLIDQYLYYLNKTQS |
| | NSGALQQSRLLFSQAGPTSMSLQAKNWLPGPCYRQQRLSKQANDNNNSNF |
| | PWTAATKYHLNGRDSLVNPGPAMASHKDDEEKFFPMHGTLIFGKQGTNAN |
| | DADLDNVMITDEEEIRTTNPVATEQYGYVSNNLQNSNTGPTTGTVNHRGA |
| | LPGMVWQDRDVYLQGPIWAKIPHTDGHFHPSPLMGGFGLKHPPPQIMIKN |
| | TPVPANPPTNFSSAKFASFITQYSTGQVSVEIEWELQKENSKRWNPEIQY |
| | TSNYNKSVNVDFTVDTNGVYSEPRPIGTRYLTRNL |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLKAGDNPYLRYNHADAEF |
| 132 | QERLQEDTSFGGNLGRAVFQAKKRVLEPLGLVEEGAKTAPGKKRPVEQSP |
| (Anc80) | QEPDSSSGIGKKGQQPAXKRLNFGQTGDSESVPDPQPLGEPPAAPSGVGS |
| | NTMAXGGGAPMADNNEGADGVGNASGNWHCDSTWLGDRVITTSTRTWALP |
| | TYNNHLYKQISSQSGXSTNDNTYFGYSTPWGYFDFNRFHCHFSPRDWQRL |
| | INNNWGFRPKXLNFKLFNIQVKEVTTNDGTTTIANNLTSTVQVFTDSEYQ |
| | LPYVLGSAHQGCLPPFPADVFMIPQYGYLTLNNGSQAVGRSSFYCLEYFP |
| | SQMLRTGNNFXFSYTFEDVPFHSSYAHSQSLDRLMNPLIDQYLYYLSRTQ |
| | TTSGTAGNRXLQFSQAGPSSMANQAKNWLPGPCYRQQRVSKTXNQNNNSN |
| | FAWTGATKYHLNGRDSLVNPGPAMATHKDDEDKFFPMSGVLIFGKQGAGN |
| | SNVDLDNVMITXEEEIKTTNPVATEXYGTVATNLQSXNTAPATGTVNSQG |
| | ALPGMVWQXRDVYLQGPIWAKIPHTDGHFHPSPLMGGFGLKHPPPQILIK |
| | NTPVPANPPTTFSPAKFASFITQYSTGQVSVEIEWELQKENSKRWNPEIQ |
| | YTSNYNKSTNVDFAVDTNGVYSEPRPIGTRYLTRNL |
| | (168) . . . (168) Lys or Arg |
| | (205) . . . (205) Ala or Ser |
| | (266) . . . (266) Ala or Gly |
| | (311) . . . (311) Arg or Lys |
| | (411) . . . (411) Glu or Gln |
| | (460) . . . (460) Thr or Glu |
| | (493) . . . (493) Ala or Thr |
| | (562) . . . (562) Ser or Asn |
| | (576) . . . (576) Gln or Glu |
| | (587) . . . (587) Ser or Ala |
| | (609) . . . (609) Asn or Asp |
| SEQ ID | MAADGYLPDWLEDNLSEGIREWWDLKPGAPKPKANQQKQDDGRGLVLPGYK |
| NO: | YLGPFNGLDKGEPVNAADAAALEHDKAYDQQLKAGDNPYLRYNHADAEFQ |
| 133 | ERLQEDTSFGGNLGRAVFQAKKRVLEPLGLVEEGAKTAPGKKRPVEQSPQ |
| (Anc81 | EPDSSXGIGKKGQQPAXKRLNFGQTGDSESVPDPQPLGEPPAAPSGVGSN |
| (AKU8 | TMAAGGGAPMADNNEGADGVGNASGNWHCDSTWLGDRVITTSTRTWALPT |
| 9596)) | YNNHLYKQISXXQSGGSTNDNTYFGYSTPWGYFDFNRFHCHFSPRDWQRL |
| | INNNWGFRPKXLNFKLFNIQVKEVTTNDGTTTIANNLTSTVQVFTDSEYQ |
| | LPYVLGSAHQGCLPPFPADVFMIPQYGYLTLNNGSQAVGRSSFYCLEYFP |
| | SQMLRTGNNFXFSYTFEDVPFHSSYAHSQSLDRLMNPLIDQYLYYLSRTQ |
| | TTGGTAGNXXLQFSQAGPSSMANQAKNWLPGPCYRQQRVSKTTNQNNNSN |
| | FAWTGATKYHLNGRDSLVNPGVAMATHKDDEDRFFPSSGVLIFGKQGAGN |
| | XNVDXXNVMITXEEEIKTTNPVATEEYGXVATNLQSXNTAPQTGTVNSQG |
| | ALPGMVWQNRDVYLQGPIWAKIPHTDGNFHPSPLMGGFGLKHPPPQILIK |
| | NTPVPANPPTTFXPAKFASFITQYSTGQVSVEIEWELQKENSKRWNPEIQ |
| | YTSNYNKSTNVDFAVDTEGVYSEPRPIGTRYLTRNL |
| | (157) . . . (157) Thr or Ser |
| | (168) . . . (168) Lys or Arg |
| | (262) . . . (262) Asn or Ser |
| | (263) . . . (263) Ser or His |
| | (312) . . . (312) Arg or Lys |
| | (412) . . . (412) Glu or Gln |
| | (460) . . . (460) Arg or Gln |
| | (461) . . . (461) Thr or Glu |
| | (552) . . . (552) Asp or Ser |
| | (556) . . . (556) Leu or Tyr |
| | (557) . . . (557) Asp or Ser |
| | (563) . . . (563) Ser or Asn |
| | (580) . . . (580) Val or Ile |
| | (588) . . . (588) Asn or Ser |
| | (664) . . . (664) Ser or Thr |
| SEQ ID | MAADGYLPDWLEDNLSEGIREWWDLKPGAPKPKANQQKQDDGRGLVLPGYK |
| NO: | YLGPFNGLDKGEPVNAADAAALEHDKAYDQQLKAGDNPYLRYNHADAEFQ |
| 134 | ERLQEDTSFGGNLGRAVFQAKKRVLEPLGLVEEGAKTAPGKKRPVEQSPQ |
| (Anc82 | REPDSSXGIGKKGQQPAXKRLNFGQTGDSESVPDPQPLGEPPAAPSGVGS |
| (AKU8 | NTMAAGGGAPMADNNEGADGVGNSSGNWHCDSTWLGDRVITTSTRTWALP |
| 9597)) | TYNNHLYKQISNGTSGGSTNDNTYFGYSTPWGYFDFNRFHCHFSPRDWQR |
| | LINNNWGFRPKRLNFKLFNIQVKEVTTNEGTKTIANNLTSTVQVFTDSEY |
| | QLPYVLGSAHQGCLPPFPADVFMIPQYGYLTLNNGSQAVGRSSFYCLEYF |
| | PSQMLRTGNNFQFSYTFEDVPFHSSYAHSQSLDRLMNPLIDQYLYYLSRT |
| | QTTGGTAGTQTLQFSQAGPSSMANQAKNWLPGPCYRQQRVSTTTNQNNNS |
| | NFAWTGATKYHLNGRDSLVNPGVAMATHKDDEDRFFPSSGVLIFGKQGAG |
| | NDNVDYSNVMITXEEEIKTTNPVATEEYGVVATNLQSANTAPQTGTVNSQ |
| | GALPGMVWQNRDVYLQGPIWAKIPHTDGNFHPSPLMGGFGLKHPPPQILI |
| | KNTPVPADPPTTFNQAKLNSFITQYSTGQVSVEIEWELQKENSKRWNPEI |
| | QYTSNYYKSTNVDFAVNTEGVYSEPRPIGTRYLTRNL |
| | (158) . . . (158) Thr or Ser |
| | (169) . . . (169) Lys or Arg |
| | (564) . . . (564) Ser or Asn |
| SEQ ID | MAADGYLPDWLEDNLSEGIREWWDLKPGAPKPKANQQKQDDGRGLVLPGYK |
| NO: | YLGPFNGLDKGEPVNAADAAALEHDKAYDQQLKAGDNPYLRYNHADAEFQ |
| 135 | ERLQEDTSFGGNLGRAVFQAKKRVLEPLGLVEEGAKTAPGKKRPVEQSPQ |
| (Anc83 | REPDSSXGIGKKGQQPAXKRLNFGQTGDSESVPDPQPLGEPPAAPSGVGS |
| (AKU8 | NTMAAGGGAPMADNNEGADGVGSSSGNWHCDSTWLGDRVITTSTRTWALP |
| 9598)) | TYNNHLYKQISNGTSGGSTNDNTYFGYSTPWGYFDFNRFHCHFSPRDWQR |
| | LINNNWGFRPKRLXFKLFNIQVKEVTQNEGTKTIANNLTSTIQVFTDSEY |
| | QLPYVLGSAHQGCLPPFPADVFMIPQYGYLTLNNGSQAVGRSSFYCLEYF |
| | PSQMLRTGNNFXFSYTFEDVPFHSSYAHSQSLDRLMNPLIDQYLYYLSRT |
| | QTTGGTAGTQTLQFSQAGPSXMANQAKNWLPGPCYRQQRVSTTTSQNNNS |
| | NFAWTGATKYHLNGRDSLVNPGVAMATHKDDEXRFFPSSGXLIFGKQGAG |
| | KDNVDYSNVMLTSEEEIKTTNPVATEEYGVVADNLQQQNTAPQXGTVNSQ |
| | GALPGMVWQNRDVYLQGPIWAKIPHTDGNFHPSPLMGGFGLKHPPPQILI |
| | KNTPVPADPPTTFNQAKLNSFITQYSTGQVSVEIEWELQKENSKRWNPEI |
| | QYTSNYYKSTNVDFAVNTEGVYSEPRPIGTRYLTRNL |
| | (158) . . . (158) Thr or Ser |
| | (169) . . . (169) Arg or Lys |
| | (315) . . . (315) Asn or Ser |
| | (413) . . . (413) Gln or Glu |
| | (472) . . . (472) Asn, Thr or Ser |
| | (534) . . . (534) Asp or Glu |
| | (542) . . . (542) Ile or Val |
| | (595) . . . (595) Ile or Val |
| SEQ ID | MAADGYLPDWLEDNLSEGIREWWDLKPGAPKPKANQQKQDDGRGLVLPGYK |
| NO: | YLGPFNGLDKGEPVNAADAAALEHDKAYDQQLKAGDNPYLRYNHADAEFQ |
| 136 | ERLQEDTSFGGNLGRAVFQAKKRVLEPLGLVEEGAKTAPGKKRPVEPSPQ |
| (Anc84 | RSPDSSTGIGKKGQQPAXKRLNFGQTGDSESVPDPQPIGEPPAAPSGVGS |
| (AKU8 | GTMAAGGGAPMADNNEGADGVGSSSGNWHCDSTWLGDRVITTSTRTWALP |
| 9599)) | TYNNHLYKQISNGTSGGSTNDNTYFGYSTPWGYFDFNRFHCHFSPRDWQR |
| | LINNNWGFRPKRLXFKLFNIQVKEVTQNEGTKTIANNLTSTIQVFTDSEY |
| | QLPYVLGSAHQGCLPPFPADVFMIPQYGYLTLNNGSQAVGRSSFYCLEYF |
| | PSQMLRTGNNFEFSYTFEDVPFHSSYAHSQSLDRLMNPLIDQYLYYLSRT |
| | QSTGGTAGTQQLLFSQAGPSNMSAQAKNWLPGPCYRQQRVSTTLSQNNNS |
| | NFAWTGATKYHLNGRDSLVNPGVAMATHKDDEXRFFPSSGXLMFGKQGAG |
| | KDNVDYSNVMLTSEEEIKTTNPVATEQYGVVADNLQQQNTAPIVGAVNSQ |
| | GALPGMVWQNRDVYLQGPIWAKIPHTDGNFHPSPLMGGFGLKHPPPQILI |
| | KNTPVPADPPTTFNQAKLNSFITQYSTGQVSVEIEWELQKENSKRWNPEI |
| | QYTSNYYKSTNVDFAVNTEGVYSEPRPIGTRYLTRNL |
| | (169) . . . (169) Arg or Lys |
| | (315) . . . (315) Asn or Ser |
| | (534) . . . (534) Asp or Glu |
| | (542) . . . (542) Ile or Val |
| SEQ ID | MAADGYLPDWLEDNLSEGIREWWDLKPGAPKPKANQQKQDDGRGLVLPGYK |
| NO: | YLGPFNGLDKGEPVNAADAAALEHDKAYDQQLKAGDNPYLRYNHADAEFQE |
| 137 | RLQEDTSFGGNLGRAVFQAKKRVLEPLGLVEEGAKTAPGKKRPVEPSPQRS |
| (Anc94) | PDSSTGIGKKGQQPAKKRLNFGQTGDSESVPDPQPIGEPPAGPSGLGSGTM |
| | AAGGGAPMADNNEGADGVGSSSGNWHCDSTWLGDRVITTSTRTWALPTYNN |
| | HLYKQISNGTSGGSTNDNTYFGYSTPWGYFDFNRFHCHFSPRDWQRLINNN |
| | WGFRPKRLNFKLFNIQVKEVTQNEGTKTIANNLTSTIQVFTDSEYQLPYVL |
| | GSAHQGCLPPFPADVFMIPQYGYLTLNNGSQAVGRSSFYCLEYFPSQMLRT |
| | GNNFEFSYTFEDVPFHSSYAHSQSLDRLMNPLIDQYLYYLSRTQSTGGTAG |
| | TQQLLFSQAGPXNMSAQAKNWLPGPCYRQQRVSTTLSQNNNSNFAWTGATK |
| | YHLNGRDSLVNPGVAMATHKDDEERFFPSSGVLMFGKQGAGKDNVDYSSVM |
| | LTSEEEIKTTNPVATEQYGVVADNLQQQNTAPIVGAVNSQGALPGMVWQNR |
| | DVYLQGPIWAKIPHTDGNFHPSPLMGGFGLKHPPPQILIKNTPVPADPPTT |
| | FSQAKLASFITQYSTGQVSVEIEWELQKENSKRWNPEIQYTSNYYKSTNVD |
| | FAVNTEGTYSEPRPIGTRYLTRNL |
| | (471) . . . (471) Ser or Asn |
| SEQ ID | MAADGYLPDWLEDNLSEGIREWWDLKPGAPKPKANQQKQDDGRGLVLPGYK |
| NO: | YLGPFNGLDKGEPVNAADAAALEHDKAYDQQLKAGDNPYLRYNHADAEFQER |
| 138 | LQEDTSFGGNLGRAVFQAKKRVLEPLGLVEEGAKTAPGKKRPVEQSPQEPDS |
| (Anc110 | SXLGIGKTGQQPAXKRLNFGQTGDSESVPDPQPLGEPPAAPSGVGSNTMASG |
| (AKU89600)) | GGAPMADNNEGADGVGNSSGNWHCDSTWLGDRVITTSTRTWALPTYNNHLYK |
| | QISNGTSGGSTNDNTYFGYSTPWGYFDFNRFHCHFSPRDWQRLINNNWGFRP |
| | KRLNFKLFNIQVKEVTTNEGTKTIANNLTSTVQVFTDSEYQLPYVLGSAHQG |
| | CLPPFPADVFMIPQYGYLTLNNGSQAVGRSSFYCLEYFPSQMLRTGNNFQFS |
| | YTFEDVPFHSSYAHSQSLDRLMNPLIDQYLYYLSRTQTTGTXGTQTLXFSQA |
| | GPSSMANQARNWVPGPCYRQQRVSTTTNQNNNSNFAWTGAXKXXLNGRDSLM |
| | NPGVAMASHKDDEDRFFPSSGVLIFGKQGAGNDNVDYSXVMITNEEEIKTTN |
| | PVATEEYGAVATNXQXLANTQAQTGLVHNQGVLPGMVWQNRDVYLQGPIWAK |
| | IPHTDGNFHPSPLMGGFGLKHPPPQILIKNTPVPADPPTTFNQAKLNSFITQ |
| | YSTGQVSVEIEWELQKENSKRWNPEIQYTSNYYKSTNVDFAVNTEGVYSEPR |
| | PIGTRYLTRNL |
| | (157) . . . (157) Ser or Thr |
| | (169) . . . (169) Lys or Arg |
| | (457) . . . (457) Ala or Gly |
| | (463) . . . (463) Gln or Ala |
| | (508) . . . (508) Thr or Ala |
| | (510) . . . (510) Tyr or Phe |
| | (511) . . . (511) His or Lys |
| | (558) . . . (558) Gln or Asn |
| | (585) . . . (585) Asn or His |
| | (587) . . . (587) Ser or Ala |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLKAGDNPYLRYNHADAEF |
| 139 | QERLQEDTSFGGNLGRAVFQAKKRVLEPLGLVEEGAKTAPGKKRPVEXSP |
| (Anc113 | QRSPDSSTGIGKKGQQPAXKRLNFGQTGDSESVPDPQPLGEPPAAPSGVG |
| (AKU89601)) | SGTMAAGGGAPMADNNEGADGVGNASGNWHCDSTWLGDRVITTSTRTWAL |
| | PTYNNHLYKQISSQSAGSTNDNTYFGYSTPWGYFDFNRFHCHFSPRDWQR |
| | LINNNWGFRPKKLXFKLFNIQVKEVTTNDGVTTIANNLTSTVQVFSDSEY |
| | QLPYVLGSAHQGCLPPFPADVFMIPQYGYLTLNNGSQSVGRSSFYCLEYF |
| | PSQMLRTGNNFEFSYTFEDVPFHSSYAHSQSLDRLMNPLIDQYLYYLART |
| | QSTTGGTAGNRELQFXQAGPSTMABQAKNWLPGPCYRQQRVSKTLDQNNN |
| | SNFAWTGATKYHLNGRNSLVNPGVAMATHKDDEDRFFPSSGVLIFGKTGA |
| | ANKTTLENVLMTXEEEIKTTNPVATEEYGXVSSNLQSXNTAPQTQTVNSQ |
| | GALPGMVWQNRDVYLQGPIWAKIPHTDGNFHPSPLMGGFGLKHPPPQILI |
| | KNTPVPANPPEVFTPAKFASFITQYSTGQVSVEIEWELQKENSKRWNPEI |
| | QYTSNYDKSTNVDFAVDSEGVYSEPRPIGTRYLTRNL |
| | (148) . . . (148) Pro or Gln |
| | (169) . . . (169) Lys or Arg |
| | (314) . . . (314) Arg or Asn |
| | (466) . . . (466) Tyr or His |
| | (563) . . . (563) Asn or Ser |
| | (580) . . . (580) Val or Ile |
| | (588) . . . (588) Ala or Ser |
| SEQ ID | MAADGYLPDWLEDNLSEGIREWWDLKPGAPKPKANQQKQDDGRGLVLPGYK |
| NO: | YLGPFNGLDKGEPVNAADAAALEHDKAYDQQLKAGDNPYLRYNHADAEFQ |
| 140 | ERLQEDTSFGGNLGRAVFQAKKRVLEPLGLVEEGAKTAPGKKRPVEQSPQ |
| (Anc126 | EPDSSSGIGKXGQQPAXKRLNFGQTGDSESVPDPQPLGEPPAAPSGVGSN |
| (AKU89602)) | TMASGGGAPMADNNEGADGVGNXSGNWHCDSTWLGDRVITTSTRTWALPT |
| | YNNHLYKQISSQSGASNDNHYFGYSTPWGYFDFNRFHCHFSPRDWQRLIN |
| | NNWGFRPKXLNFKLFNIQVKEVTTNDGTTTIANNLTSTVQVFTDSEYQLP |
| | YVLGSAHQGCLPPFPADVFMIPQYGYLTLNNGSQAVGRSSFYCLEYFPSQ |
| | MLRTGNNFXFSYTFEDVPFHSSYAHSQSLDRLMNPLIDQYLYYLXRTQTT |
| | SGTAQNRELXFSQAGPSSMXNQAKNWLPGPCYRQQRVSKTANDNNNSNFA |
| | WTGATKYHLNGRDSLVNPGPAMASHKDDEDKFFPMSGVLIFGKQGAGASN |
| | VDLDNVMITDEEEIKTTNPVATEQYGTVATNLQSSNTAPATGTVNSQGAL |
| | PGMVWQDRDVYLQGPIWAKIPHTDGHFHPSPLMGGFGLKHPPPQILIKNT |
| | PVPANPPTTFSPAKFASFITQYSTGQVSVEIEWELQKENSKRWNPEIQYT |
| | SNYNKSXNVDFTVDINGVYSEPRPIGTRYLTRNL |
| | (162) . . . (162) Ser or Thr |
| | (168) . . . (168) Lys or Arg |
| | (224) . . . (224) Ala or Ser |
| | (310) . . . (310) Arg or Lys |
| | (410) . . . (410) Thr or Gln |
| | (446) . . . (446) Ser or Asn |
| | (461) . . . (461) Gln or Leu |
| | (471) . . . (471) Ala or Ser |
| | (708) . . . (708) Ala or Thr |
| SEQ ID | MAADGYLPDWLEDNLSEGIREWWDLKPGAPQPKANQQHQDDXRGLVLPGY |
| NO: | KYLGPFNGLDKGEPVNEADAAALEHDKAYDQQLKAGDNPYLKYNHADAEF |
| 141 | QERLQEDTSFGGNLGRAVFQAKKRVLEPLGLVEEAAKTAPGKKRPVEQSP |
| Anc127 | QEPDSSSGIGKSGQQPAXKRLNFGQTGDSESVPDPQPLGEPPAAPSGVGS |
| (AKU89603) | NTMASGGGAPMADNNEGADGVGNSSGNWHCDSTWLGDRVITTSTRTWALP |
| | TYNNHLYKQISSQSGASNDNHYFGYSTPWGYFDFNRFHCHFSPRDWQRLI |
| | NNNWGFRPKXLNFKLFNIQVKEVTQNDGTTTIANNLTSTVQVFTDSEYQL |
| | PYVLGSAHQGCLPPFPADVFMIPQYGYLTLNNGSQAVGRSSFYCLEYFPS |
| | QMLRTGNNFXFSYTFEDVPFHSSYAHSQSLDRLMNPLIDQYLYYLXRTQT |
| | TSGTTQQSRLXFSQAGPSSMXQQAXNWLPGPCYRQQRVSKTANDNNNSNF |
| | AWTXATKYHLNGRDSLVNPGPAMASHKDDEEKFFPMHGXLIFGKQGTGAS |
| | NVDLDNVMITDEEEIRTTNPVATEQYGTVATNLQSSNTAPATGTVNSQGA |
| | LPGMVWQDRDVYLQGPIWAKIPHTDGHFHPSPLMGGFGLKHPPPQILIKN |
| | TPVPANPPTTFSPAKFASFITQYSTGQVSVEIEWELQKENSKRWNPEIQY |
| | TSNYNKSVNVDFTVDINGVYSEPRPIGTRYLTRNL |
| | (42) . . . (42) Gly or Ser |
| | (168) . . . (168) Arg or Lys |
| | (310) . . . (310) Lys or Arg |
| | (410) . . . (410) Thr or Gln |
| | (446) . . . (446) Ser or Arg |
| | (461) . . . (461) Gln or Leu |
| | (471) . . . (471) Ala or Ser |
| | (475) . . . (475) Lys or Arg |
| | (504) . . . (504) Gly or Ala |
| | (539) . . . (539) Val or Asn |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLKAGDNPYLRYNHADAEF |
| 142 | QERLQEDTSFGGNLGRAVFQAKKRVLEPLGLVEEGAKTAPGKKRPVEQSP |
| (Anc80 | QEPDSSSGIGKKGQQPARKRLNFGQTGDSESVPDPQPLGEPPAAPSGVGS |
| L65 | NTMAAGGGAPMADNNEGADGVGNASGNWHCDSTWLGDRVITTSTRTWALP |
| (AKU89595)) | TYNNHLYKQISSQSGGSTNDNTYFGYSTPWGYFDFNRFHCHFSPRDWQRL |
| | INNNWGFRPKKLNFKLFNIQVKEVTTNDGTTTIANNLTSTVQVFTDSEYQ |
| | LPYVLGSAHQGCLPPFPADVFMIPQYGYLTLNNGSQAVGRSSFYCLEYFP |
| | SQMLRTGNNFQFSYTFEDVPFHSSYAHSQSLDRLMNPLIDQYLYYLSRTQ |
| | TTSGTAGNRTLQFSQAGPSSMANQAKNWLPGPCYRQQRVSKTTNQNNNSN |
| | FAWTGATKYHLNGRDSLVNPGPAMATHKDDEDKFFPMSGVLIFGKQGAGN |
| | SNVDLDNVMITNEEEIKTTNPVATEEYGTVATNLQSANTAPATGTVNSQG |
| | ALPGMVWQDRDVYLQGPIWAKIPHTDGHFHPSPLMGGFGLKHPPPQILIK |
| | NTPVPANPPTTFSPAKFASFITQYSTGQVSVEIEWELQKENSKRWNPEIQ |
| | YTSNYNKSTNVDFAVDINGVYSEPRPIGTRYLTRNL |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLKAGDNPYLRYNHADAEF |
| 143 | QERLQEDTSFGGNLGRAVFQAKKRVLEPLGLVEEGAKTAPGKKRPVBQSP |
| (Anc80 | QEPDSSSGIGKKGQQPAKKRLNFGQTGDSESVPDPQPLGEPPAAPSGVGS |
| L1) | NTMAAGGGAPMADNNEGADGVGNASGNWHCDSTWLGDRVITTSTRTWALP |
| | TYNNHLYKQISSQSGASTNDNTYFGYSTPWGYFDFNRFHCHFSPRDWQRL |
| | INNNWGFRPKRLNFKLFNIQVKEVTTNDGTTTIANNLTSTVQVFTDSEYQ |
| | LPYVLGSAHQGCLPPFPADVFMIPQYGYLTLNNGSQAVGRSSFYCLEYFP |
| | SQMLRTGNNFEFSYTFEDVPFHSSYAHSQSLDRLMNPLIDQYLYYLSRTQ |
| | TTSGTAGNRTLQFSQAGPSSMANQAKNWLPGPCYRQQRVSKTANQNNNSN |
| | FAWTGATKYHLNGRDSLVNPGPAMATHKDDEDKFFPMSGVLIFGKQGAGN |
| | SNVDLDNVMITSEEEIKTTNPVATEQYGTVATNLQSSNTAPATGTVNSQG |
| | ALPGMVWQNRDVYLQGPIWAKIPHTDGHFHPSPLMGGFGLKHPPPQILIK |
| | NTPVPANPPTTFSPAKFASFITQYSTGQVSVEIEWELQKENSKRWNPEIQ |
| | YTSNYNKSTNVDFAVDINGVYSEPRPIGTRYLTRNL |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLKAGDNPYLRYNHADAEF |
| 144 | QERLQEDTSFGGNLGRAVFQAKKRVLEPLGLVEEGAKTAPGKKRPVEQSP |
| (Anc80 | QEPDSSSGIGKKGQQPARKRLNFGQTGDSESVPDPQPLGEPPAAPSGVGS |
| L27) | NTMAAGGGAPMADNNEGADGVGNASGNWHCDSTWLGDRVITTSTRTWALP |
| | TYNNHLYKQISSQSGGSTNDNTYFGYSTPWGYFDFNRFHCHFSPRDWQRL |
| | INNNWGFRPKRLNFKLFNIQVKEVTINDGTTTIANNLTSTVQVFTDSEYQ |
| | LPYVLGSAHQGCLPPFPADVFMIPQYGYLTLNNGSQAVGRSSFYCLEYFP |
| | SQMLRTGNNFEFSYTFEDVPFHSSYAHSQSLDRLMNPLIDQYLYYLSRTQ |
| | TTSGTAGNRTLQFSQAGPSSMANQAKNWLPGPCYRQQRVSKTANQNNNSN |
| | FAWTGATKYHLNGRDSLVNPGPAMATHKDDEDKFFPMSGVLIFGKQGAGN |
| | SNVDLDNVMITNEEEIKTTNPVATEQYGTVATNLQSANTAPATGTVNSQG |
| | ALPGMVWQDRDVYLQGPIWAKIPHTDGHFHPSPLMGGFGLKHPPPQILIK |
| | NTPVPANPPTTFSPAKFASFITQYSTGQVSVEIEWELQKENSKRWNPEIQ |
| | YTSNYNKSTNVDFAVDINGVYSEPRPIGTRYLTRNL |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLKAGDNPYLRYNHADAEF |
| 145 | QERLQEDTSFGGNLGRAVFQAKKRVLEPLGLVEEGAKTAPGKKRPVEQSP |
| (Anc80 | QEPDSSSGIGKKGQQPAKKRLNFGQTGDSESVPDPQPLGEPPAAPSGVGS |
| L33) | NTMAAGGGAPMADNNEGADGVGNASGNWHCDSTWLGDRVITTSTRTWALP |
| | TYNNHLYKQISSQSGGSTNDNTYFGYSTPWGYFDFNRFHCHFSPRDWQRL |
| | INNNWGFRPKKLNFKLFNIQVKEVTTNDGTTTIANNLTSTVQVFTDSEYQ |
| | LPYVLGSAHQGCLPPFPADVFMIPQYGYLTLNNGSQAVGRSSFYCLEYFP |
| | SQMLRTGNNFEFSYTFEDVPFHSSYAHSQSLDRLMNPLIDQYLYYLSRTQ |
| | TTSGTAGNRTLQFSQAGPSSMANQAKNWLPGPCYRQQRVSKTANQNNNSN |
| | FAWTGATKYHLNGRDSLVNPGPAMATHKDDEDKFFPMSGVLIFGKQGAGN |
| | SNVDLDNVMITSEEEIKTTNPVATEQYGTVATNLQSSNTAPATGTVNSQG |
| | ALPGMVWQNRDVYLQGPIWAKIPHTDGHFHPSPLMGGFGLKHPPPQILIK |
| | NTPVPANPPTTFSPAKFASFITQYSTGQVSVEIEWELQKENSKRWNPEIQ |
| | YTSNYNKSTNVDFAVDTNGVYSEPRPIGTRYLTRNL |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLKAGDNPYLRYNHADAEF |
| 146 | QERLQEDTSFGGNLGRAVFQAKKRVLEPLGLVEEGAKTAPGKKRPVEQSP |
| (Anc80 | QEPDSSSGIGKKGQQPAKKRLNFGQTGDSESVPDPQPLGEPPAAPSGVGS |
| L36) | NTMASGGGAPMADNNEGADGVGNASGNWHCDSTWLGDRVITTSTRTWALP |
| | TYNNHLYKQISSQSGGSTNDNTYFGYSTPWGYFDFNRFHCHFSPRDWQRL |
| | INNNWGFRPKKLNFKLFNIQVKEVTTNDGTTTIANNLTSTVQVFTDSEYQ |
| | LPYVLGSAHQGCLPPFPADVFMIPQYGYLTLNNGSQAVGRSSFYCLEYFP |
| | SQMLRTGNNFEFSYTFEDVPFHSSYAHSQSLDRLMNPLIDQYLYYLSRTQ |
| | TTSGTAGNRTLQFSQAGPSSMANQAKNWLPGPCYRQQRVSKTANQNNNSN |
| | FAWTGATKYHLNGRDSLVNPGPAMATHKDDEDKFFPMSGVLIFGKQGAGN |
| | SNVDLDNVMITSEEEIKTTNPVATEEYGTVATNLQSSNTAPATGTVNSQG |
| | ALPGMVWQNRDVYLQGPIWAKIPHTDGHFHPSPLMGGFGLKHPPPQILIK |
| | NTPVPANPPTTFSPAKFASFITQYSTGQVSVEIEWELQKENSKRWNPEIQ |
| | YTSNYNKSTNVDFAVDINGVYSEPRPIGTRYLTRNL |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLKAGDNPYLRYNHADAEF |
| 147 | QERLQEDTSFGGNLGRAVFQAKKRVLEPLGLVEEGAKTAPGKKRPVEQSP |
| (Anc80 | QEPDSSSGIGKKGQQPAKKRLNFGQTGDSESVPDPQPLGEPPAAPSGVGS |
| L44) | NTMASGGGAPMADNNEGADGVGNASGNWHCDSTWLGDRVITTSTRTWALP |
| | TYNNHLYKQISSQSGGSTNDNTYFGYSTPWGYFDFNRFHCHFSPRDWQRL |
| | INNNWGFRPKKLNFKLFNIQVKEVTTNDGTTTIANNLTSTVQVFTDSEYQ |
| | LPYVLGSAHQGCLPPFPADVFMIPQYGYLTLNNGSQAVGRSSFYCLEYFP |
| | SQMLRTGNNFQFSYTFEDVPFHSSYAHSQSLDRLMNPLIDQYLYYLSRTQ |
| | TTSGTAGNRELQFSQAGPSSMANQAKNWLPGPCYRQQRVSKTTNQNNNSN |
| | FAWTGATKYHLNGRDSLVNPGPAMATHKDDEDKFFPMSGVLIFGKQGAGN |
| | SNVDLDNVMITNEEEIKTINPVATEQYGTVATNLQSANTAPATGTVNSQG |
| | ALPGMVWQDRDVYLQGPIWAKIPHTDGHFHPSPLMGGFGLKHPPPQILIK |
| | NTPVPANPPTTFSPAKFASFITQYSTGQVSVEIEWELQKENSKRWNPEIQ |
| | YTSNYNKSTNVDFAVDINGVYSEPRPIGTRYLTRNL |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLKAGDNPYLRYNHADAEF |
| 148 | QERLQEDTSFGGNLGRAVFQAKKRVLEPLGLVEEGAKTAPGKKRPVEQSP |
| (Anc80 | QEPDSSSGIGKKGQQPAKKRLNFGQTGDSESVPDPQPLGEPPAAPSGVGS |
| L59) | NTMASGGGAPMADNNEGADGVGNASGNWHCDSTWLGDRVITTSTRTWALP |
| | TYNNHLYKQISSQSGASTNDNTYFGYSTPWGYFDFNRFHCHFSPRDWQRL |
| | INNNWGFRPKRLNEKLFNIQVKEVTINDGTTTIANNLTSTVQVFTDSEYQ |
| | LPYVLGSAHQGCLPPFPADVFMIPQYGYLTLNNGSQAVGRSSFYCLEYFP |
| | SQMLRTGNNFQFSYTFEDVPFHSSYAHSQSLDRLMNPLIDQYLYYLSRTQ |
| | TTSGTAGNRELQFSQAGPSSMANQAKNWLPGPCYRQQRVSKTTNQNNNSN |
| | FAWTGATKYHLNGRDSLVNPGPAMATHKDDEDKFFPMSGVLIFGKQGAGN |
| | SNVDLDNVMITNEEEIKTTNPVATBEYGTVATNLQSANTAPATGTVNSQG |
| | ALPGMVWQNRDVYLQGPIWAKIPHTDGHFHPSPLMGGFGLKHPPPQILIK |
| | NTPVPANPPTTFSPAKFASFITQYSTGQVSVEIEWELQKENSKRWNPEIQ |
| | YTSNYNKSTNVDFAVDINGVYSEPRPIGTRYLTRNL |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLKAGDNPYLRYNHADAEF |
| 149 | QERLQEDTSFGGNLGRAVFQAKKRVLEPLGLVEEGAKTAPGKKRPVEQSP |
| (Anc80 | QEPDSSSGIGKKGQQPARKRLNFGQTGDSESVPDPQPLGEPPAAPSGVGS |
| L60) | NTMAAGGGAPMADNNEGADGVGNASGNWHCDSTWLGDRVITTSTRTWALP |
| | TYNNHLYKQISSQSGGSTNDNTYFGYSTPWGYFDFNRFHCHFSPRDWQRL |
| | INNNWGFRPKRLNEKLFNIQVKEVTTNDGTTTIANNLTSTVQVFTDSEYQ |
| | LPYVLGSAHQGCLPPFPADVFMIPQYGYLTLNNGSQAVGRSSFYCLEYFP |
| | SQMLRTGNNFEFSYTFEDVPFHSSYAHSQSLDRLMNPLIDQYLYYLSRTQ |
| | TTSGTAGNRELQFSQAGPSSMANQAKNWLPGPCYRQQRVSKTTNQNNNSN |
| | FAWTGATKYHLNGRDSLVNPGPAMATHKDDEDKFFPMSGVLIFGKQGAGN |
| | SNVDLDNVMITSEEEIKTTNPVATEEYGTVATNLQSSNTAPATGTVNSQG |
| | ALPGMVWQERDVYLQGPIWAKIPHTDGHFHPSPLMGGFGLKHPPPQILIK |
| | NTPVPANPPTTFSPAKFASFITQYSTGQVSVEIEWELQKENSKRWNPEIQ |
| | YTSNYNKSTNVDFAVDINGVYSEPRPIGTRYLTRNL |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLKAGDNPYLRYNHADAEF |
| 150 | QERLQEDTSFGGNLGRAVFQAKKRVLEPLGLVEEGAKTAPGKKRPVEQSP |
| (Anc80 | QEPDSSSGIGKKGQQPARKRLNFGQTGDSESVPDPQPLGEPPAAPSGVGS |
| L62) | NTMASGGGAPMADNNEGADGVGNASGNWHCDSTWLGDRVITTSTRTWALP |
| | TYNNHLYKQISSQSGGSTNDNTYFGYSTPWGYFDFNRFHCHFSPRDWQRL |
| | INNNWGFRPKKLNFKLFNIQVKEVTTNDGTTTIANNLTSTVQVFTDSEYQ |
| | LPYVLGSAHQGCLPPFPADVFMIPQYGYLTLNNGSQAVGRSSFYCLEYFP |
| | SQMLRTGNNFEFSYTFEDVPFHSSYAHSQSLDRLMNPLIDQYLYYLSRTQ |
| | TTSGTAGNRELQFSQAGPSSMANQAKNWLPGPCYRQQRVSKTTNQNNNSN |
| | FAWTGATKYHLNGRDSLVNPGPAMATHKDDEDKFFPMSGVLIFGKQGAGN |
| | SNVDLDNVMITSEEEIKTTNPVATEEYGTVATNLQSANTAPATGTVNSQG |
| | ALPGMVWQDRDVYLQGPIWAKIPHTDGHFHPSPLMGGFGLKHPPPQILIK |
| | NTPVPANPPTTFSPAKFASFITQYSTGQVSVEIEWELQKENSKRWNPEIQ |
| | YTSNYNKSTNVDFAVDINGVYSEPRPIGTRYLTRNL |
| SEQ ID | MAADGYLPDWLEDNLSEGIREWWDLKPGAPKPKANQQKQDDGRGLVLPGY |
| NO: | KYLGPFNGLDKGEPVNAADAAALEHDKAYDQQLKAGDNPYLRYNHADAEF |
| 151 | QERLQEDTSFGGNLGRAVFQAKKRVLEPLGLVEEGAKTAPGKKRPVEQSP |
| (Anc82 | QREPDSSTGIGKSGQQPAKKRLNFGQTGDSESVPDPQPLGEPPAAPSGVG |
| DI) | SNTMASGGGAPMADNNEGADGVGNSSGNWHCDSTWLGDRVITTSTRTWAL |
| | PTYNNHLYKQISNGTSGGSTNDNTYFGYSTPWGYFDFNRFHCHFSPRDWQ |
| | RLINNNWGFRPKRLNFKLFNIQVKEVTTNEGTKTIANNLTSTVQVFTDSE |
| | YQLPYVLGSAHQGCLPPFPADVFMIPQYGYLTLNNGSQAVGRSSFYCLEY |
| | FPSQMLRTGNNFQFSYTFEDVPFHSSYAHSQSLDRLMNPLIDQYLYYLSR |
| | TQTTGGTAGTQTLQFSQAGPSSMANQARNWVPGPCYRQQRVSTTTNQNNN |
| | SNFAWTGATKYHLNGRDSLMNPGVAMASHKDDEDRFFPSSGVLIFGKQGA |
| | GNDNVDYSNVMITSEEEIKTTNPVATEEYGVVATNHQSANTQAQTGTVQN |
| | QGILPGMVWQNRDVYLQGPIWAKIPHTDGNFHPSPLMGGFGLKHPPPQIL |
| | IKNTPVPADPPTTFNQAKLNSFITQYSTGQVSVEIEWELQKENSKRWNPE |
| | IQYTSNYYKSTNVDFAVNTEGVYSEPRPIGTRYLTRNL |
| SEQ ID | MAADGYLPDWLEDNLSEGIREWWDLKPGAPKPKANQQKQDNGRGLVLPGY |
| NO: | KYLGPFNGLDKGEPVNAADAAALEHDKAYDQQLQAGDNPYLRYNHADAEF |
| 152 | QERLQEDTSFGGNLGRAVFQAKKRVLEPLGLVESPVKTAPGKKRPVEPSP |
| (AAV | QRSPDSSTGIGKKGQQPAKKRLNFGQTGDSESVPDPQPIGEPPAGPSGLG |
| rh. 74) | SGTMAAGGGAPMADNNEGADGVGSSSGNWHCDSTWLGDRVITTSTRTWAL |
| | PTYNNHLYKQISNGTSGGSTNDNTYFGYSTPWGYFDFNRFHCHFSPRDWQ |
| | RLINNNWGFRPKRLNFKLFNIQVKEVTQNEGTKTIANNLTSTIQVFTDSE |
| | YQLPYVLGSAHQGCLPPFPADVFMIPQYGYLTLNNGSQAVGRSSFYCLEY |
| | FPSQMLRTGNNFEFSYNFEDVPFHSSYAHSQSLDRLMNPLIDQYLYYLSR |
| | TQSTGGTAGTQQLLFSQAGPNNMSAQAKNWLPGPCYRQQRVSTTLSQNNN |
| | SNFAWTGATKYHLNGRDSLVNPGVAMATHKDDEERFFPSSGVLMFGKQGA |
| | GKDNVDYSSVMLTSEEEIKTTNPVATEQYGVVADNLQQQNAAPIVGAVNS |
| | QGALPGMVWQNRDVYLQGPIWAKIPHTDGNFHPSPLMGGFGLKHPPPQIL |
| | IKNTPVPADPPTTFNQAKLASFITQYSTGQVSVEIEWELQKENSKRWNPE |
| | IQYTSNYYKSTNVDFAVNTEGTYSEPRPIGTRYLTRNL |
| SEQ ID | RGDLRVS |
| NO: | |
| 153 | |
| SEQ ID | RGDAVGV |
| NO: | |
| 154 | |
| SEQ ID | RGDFTPTS |
| NO: | |
| 155 | |
| SEQ ID | RGDLGLS |
| NO: | |
| 156 | |
| SEQ ID | RGDMSRE |
| NO: | |
| 157 | |
| SEQ ID | MAADGYLPDWLEDNLSEGIREWWALKPGAPQPKANQQHQDNARGLVLPGY |
| NO: | KYLGPGNGLDKGEPVNAADAAALEHDKAYDQQLKAGDNPYLKYNHADAEF |
| 158 | QERLKEDTSFGGNLGRAVFQAKKRLLEPLGLVEEAAKTAPGKKRPVEQSP |
| (AAV9- | QEPDSSAGIGKSGAQPAKKRLNFGQTGDTESVPDPQPIGEPPAAPSGVGS |
| decol) | LTMASGGGAPVADNNEGADGVGSSSGNWHCDSQWLGDRVITTSTRTWALP |
| | TYNNHLYKQISNSTSGGSSNDNAYFGYSTPWGYFDFNRFHCHFSPRDWQR |
| | LINNNWGFRPKRLNFKLFNIQVKEVTDNNGVKTIANNLTSTVQVFTDSDY |
| | QLPYVLGSAHEGCLPPFPADVFMIPQYGYLTLNDGSQAVGRSSFYCLEYF |
| | PSQMLRTGNNFQFSYEFENVPFHSSYAHSQSLDRLMNPLIDQYLYYLSKT |
| | INGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSAQRGDLLLSAQAQT |
| | GWVQNQGILPGMVWQDRDVYLQGPIWAKIPHTDGNFHPSPLMGGFGMKHP |
| | PPQILIKNTPVPADPPTAFNKDKLNSFITQYSTGQVSVEIEWELQKENSK |
| | RWNPEIQYTSNYYKSNNVEFAVNTEGVYSEPRPIGTRYLTRNL |
| SEQ ID | MAADGYLPDWLEDNLSEGIREWWALKPGAPQPKANQQHQDNARGLVLPGY |
| NO: | KYLGPGNGLDKGEPVNAADAAALEHDKAYDQQLKAGDNPYLKYNHADAEF |
| 159 | QERLKEDTSFGGNLGRAVFQAKKRLLEPLGLVEEAAKTAPGKKRPVEQSP |
| (AAV9. | QEPDSSAGIGKSGAQPAKKRLNFGQTGDTESVPDPQPIGEPPAAPSGVGS |
| decol- | LTMASGGGAPVADNNEGADGVGSSSGNWHCDSQWLGDRVITTSTRTWALP |
| mut1) | TYNNHLYKQISNSTSGASTNDNAYFGYSTPWGYFDFNRFHCHFSPRDWQR |
| | LINNNWGFRPKRLNFKLFNIQVKEVTDNNGVKTIANNLTSTVQVFTDSDY |
| | QLPYVLGSAHEGCLPPFPADVFMIPQYGYLTLNDGSQAVGRSSFYCLEYF |
| | PSQMLRTGNNFQFSYEFENVPFHSSYAHSQSLDRLMNPLIDQYLYYLSKT |
| | INGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSAQRGDLLLSAQAQT |
| | GWVQNQGILPGMVWQDRDVYLQGPIWAKIPHTDGNFHPSPLMGGFGMKHP |
| | PPQILIKNTPVPADPPTAFNKDKLNSFITQYSTGQVSVEIEWELQKENSK |
| | RWNPEIQYTSNYYKSNNVEFAVNTEGVYSEPRPIGTRYLTRNL |
| SEQ ID | NSTSGASS |
| NO: | |
| 160 | |
| SEQ ID | NSTSGGST |
| NO: | |
| 161 | |
| SEQ ID | NSTSGAST |
| NO: | |
| 162 | |
| SEQ ID | MAADGYLPDWLEDNLSEGIREWWALKPGAPQPKANQQHQDNARGLVLPGY |
| NO: | KYLGPGNGLDKGEPVNAADAAALEHDKAYDQQLKAGDNPYLKYNHADAEF |
| 163 | QERLKEDTSFGGNLGRAVFQAKKRLLEPLGLVEEAAKTAPGKKRPVEQSP |
| (AAV9- | QEPDSSAGIGKSGAQPAKKRLNFGQTGDTESVPDPQPIGEPPAAPSGVGS |
| mut1) | LTMASGGGAPVADNNEGADGVGSSSGNWHCDSQWLGDRVITTSTRTWALP |
| | TYNNHLYKQISNSTSGASTNDNAYFGYSTPWGYFDFNRFHCHFSPRDWQR |
| | LINNNWGFRPKRLNEKLFNIQVKEVTDNNGVKTIANNLTSTVQVFTDSDY |
| | QLPYVLGSAHEGCLPPFPADVFMIPQYGYLTLNDGSQAVGRSSFYCLEYF |
| | PSQMLRTGNNFQFSYEFENVPFHSSYAHSQSLDRLMNPLIDQYLYYLSKT |
| | INGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSAQAQAQTGWVQNQG |
| | ILPGMVWQDRDVYLQGPIWAKIPHTDGNFHPSPLMGGFGMKHPPPQILIK |
| | NTPVPADPPTAFNKDKLNSFITQYSTGQVSVEIEWELQKENSKRWNPEIQ |
| | YTSNYYKSNNVEFAVNTEGVYSEPRPIGTRYLTRNL |
| SEQ ID | RDLTEAVPRLPGETLITDKEVIYICPFNGPIKGRVYITNYRLYLRSLETD |
| NO: | SSLILDVPLGVISRIEKMGGATSRGENSYGLDITCKDMRNLRFALKQEGH |
| 164 | SRRDMFEILTRYAFPLAHSLPLFAFLNEEKFNVDGWTVYNPVEEYRRQGL |
| (A1) | PNHHWRITFINKCYELCDTYPALLVVPYRASDDDLRRVATFRSRNRIPVL |
| | SWIHPENKTVIVRCSQPLVGMSGKRNKDDEKYLDVIRETNKQISKLTIYD |
| | ARPSVNAVANKATGGGYESDDAYHNAELFFLDIHNIHVMRESLKKVKDIV |
| | YPNVEESHWLSSLESTHWLEHIKLVLTGAIQVADKVSSGKSSVLVHCSDG |
| | WDRTAQLTSLAMLMLDSFYRSIEGFEILVQKEWISFGHKFASRIGHGDKN |
| | HTDADRSPIFLQFIDCVWQMSKQFPTAFEFNEQFLIIILDHLYSCRFGTF |
| | LFNCESAR |
| SEQ ID | MASASTSKYNSHSLENESIKRTSRDGVNRDLTEAVPRLPGETLITDKEVI |
| NO: | YICPFNGPIKGRVYITNYRLYLRSLETDSSLILDVPLGVISRIEKMGGAT |
| 165 | SRGENSYGLDITCKDMRNLRFALKQEGHSRRDMFEILTRYAFPLAHSLPL |
| (MTM1 | FAFLNEEKFNVDGWTVYNPVEEYRRQGLPNHHWRITFINKCYELCDTYPA |
| amino | LLVVPYRASDDDLRRVATFRSRNRIPVLSWIHPENKTVIVRCSQPLVGMS |
| acid | GKRNKDDEKYLDVIRETNKQISKLTIYDARPSVNAVANKATGGGYESDDA |
| sequence: | YHNAELFFLDIHNIHVMRESLKKVKDIVYPNVEESHWLSSLESTHWLEHI |
| A2) | KLVLTGAIQVADKVSSGKSSVLVHCSDGWDRTAQLTSLAMLMLDSFYRSI |
| | EGFEILVQKEWISFGHKFASRIGHGDKNHTDADRSPIFLQFIDCVWQMSK |
| | QFPTAFEFNEQFLIULDHLYSCRFGTFLENCESARERQKVTERTVSLWSL |
| | INSNKEKFKNPFYTKEINRVLYPVASMRHLELWVNYYIRWNPRIKQQQPN |
| | PVEQRYMELLALRDEYIKRLEELQLANSAKLSDPPTSPSSPSQMMPHVQT |
| | HF |
| SEQ ID | ATGGCTTCTGCATCAACTTCTAAATATAATTCACACTCCTTGGAGAATGA |
| NO: | GTCTATTAAGAGGACGTCTCGAGATGGAGTCAATCGAGATCTCACTGAGG |
| 166 | CTGTTCCTCGACTTCCAGGAGAAACACTAATCACTGACAAAGAAGTTATT |
| (A3: | TACATATGTCCTTTCAATGGCCCCATTAAGGGAAGAGTTTACATCACAAA |
| MTM1 | TTATCGTCTTTATTTAAGAAGTTTGGAAACGGATTCTTCTCTAATACTTG |
| coding | ATGTTCCTCTGGGTGTGATCTCGAGAATTGAAAAAATGGGAGGCGCGACA |
| seq) | AGTAGAGGAGAAAATTCCTATGGTCTAGATATTACTTGTAAAGACATGAG |
| | AAACCTGAGGTTCGCTTTGAAACAGGAAGGCCACAGCAGAAGAGATATGT |
| | TTGAGATCCTCACGAGATACGCGTTTCCCCTGGCTCACAGTCTGCCATTA |
| | TTTGCATTTTTAAATGAAGAAAAGTTTAACGTGGATGGATGGACAGTTTA |
| | CAATCCAGTGGAAGAATACAGGAGGCAGGGCTTGCCCAATCACCATTGGA |
| | GAATAACTTTTATTAATAAGTGCTATGAGCTCTGTGACACTTACCCTGCT |
| | CTTTTGGTGGTTCCGTATCGTGCCTCAGATGATGACCTCCGGAGAGTTGC |
| | AACTTTTAGGTCCCGAAATCGAATTCCAGTGCTGTCATGGATTCATCCAG |
| | AAAATAAGACGGTCATTGTGCGTTGCAGTCAGCCTCTTGTCGGTATGAGT |
| | GGGAAACGAAATAAAGATGATGAGAAATATCTCGATGTTATCAGGGAGAC |
| | TAATAAACAAATTTCTAAACTCACCATTTATGATGCAAGACCCAGCGTAA |
| | ATGCAGTGGCCAACAAGGCAACAGGAGGAGGATATGAAAGTGATGATGCA |
| | TATCATAACGCCGAACTTTTCTTCTTAGACATTCATAATATTCATGTTAT |
| | GCGGGAATCTTTAAAAAAAGTGAAGGACATTGTTTATCCTAATGTAGAAG |
| | AATCTCATTGGTTGTCCAGTTTGGAGTCTACTCATTGGTTAGAACATATC |
| | AAGCTCGTTTTGACAGGAGCCATTCAAGTAGCAGACAAAGTTTCTTCAGG |
| | GAAGAGTTCAGTGCTTGTGCATTGCAGTGACGGATGGGACAGGACTGCTC |
| | AGCTGACATCCTTGGCCATGCTGATGTTGGATAGCTTCTATAGGAGCATT |
| | GAAGGGTTCGAAATACTGGTACAAAAAGAATGGATAAGTTTTGGACATAA |
| | ATTTGCATCTCGAATAGGTCATGGTGATAAAAACCACACCGATGCTGACC |
| | GTTCTCCTATTTTTCTCCAGTTTATTGATTGTGTGTGGCAAATGTCAAAA |
| | CAGTTCCCTACAGCTTTTGAATTCAATGAACAATTTTTGATTATAATTTT |
| | GGATCATCTGTATAGTTGCCGATTTGGTACTTTCTTATTCAACTGTGAAT |
| | CTGCTCGAGAAAGACAGAAGGTTACAGAAAGGACTGTTTCTTTATGGTCA |
| | CTGATAAACAGTAATAAAGAAAAATTCAAAAACCCCTTCTATACTAAAGA |
| | AATCAATCGAGTTTTATATCCAGTTGCCAGTATGCGTCACTTGGAACTCT |
| | GGGTGAATTACTACATTAGATGGAACCCCAGGATCAAGCAACAACAGCCG |
| | AATCCAGTGGAGCAGCGTTACATGGAGCTCTTAGCCTTACGCGACGAATA |
| | CATAAAGCGGCTTGAGGAACTGCAGCTCGCCAACTCTGCCAAGCTTTCTG |
| | ATCCCCCAACTTCACCTTCCAGTCCTTCGCAAATGATGCCCCATGTGCAA |
| | ACTCACTTTTAA |
| SEQ ID | TTAAAAGTGGGTCTGGACGTGGGGCATCATCTGTGAGGGGCTGCTTGGTG |
| NO: 167 | AAGTAGGTGGGTCGGACAGCTTGGCGCTATTGGCCAGCTGCAGCTCCTCC |
| (A4) | AGCCTCTTGATATACTCATCTCTCAGGGCCAGCAGCTCCATGTACCGCTG |
| (Genscript | CTCCACAGGGTTGGGCTGCTGCTGCTTGATCCGGGGATTCCAGCGGATAT |
| codon | AGTAGTTCACCCACAGCTCCAGGTGTCTCATGGATGCCACAGGATACAGC |
| optimized | ACGCGATTGATCTCCTTTGTGTAGAAGGGGTTCTTGAACTTCTCCTTATT |
| MTM1) | GCTGTTGATCAGGCTCCACAGAGACACTGTGCGCTCGGTCACCTTCTGCC |
| | TCTCTCTGGCAGACTCACAATTAAACAGGAATGTGCCGAACCTGCAGGAG |
| | TACAGGTGGTCCAGGATGATGATCAGAAACTGCTCGTTAAACTCGAAGGC |
| | GGTTGGGAACTGCTTGGACATCTGCCACACGCAGTCGATAAACTGCAGGA |
| | AGATAGGGCTGCGGTCGGCATCGGTGTGATTCTTATCGCCGTGGCCGATC |
| | CTGGAGGCAAACTTGTGGCCGAAGCTGATCCACTCCTTCTGCACCAGGAT |
| | CTCAAAGCCCTCGATAGATCTATAGAAGGAGTCCAGCATCAGCATaGCCA |
| | GGCTTGTCAGCTGTGCTGTTCTGTCCCACCCATCGGAACAGTGCAC |
| | CAGCACGGAGCTCTTGCCAGAGGACACCTTATCGGCCACCTGGATGGCGC |
| | CTGTCAGCACCAGCTTGATGTGCTCCAGCCAGTGGGTAGACTCCAGGCTA |
| | GACAGCCAGTGGGACTCCTCCACGTTTGGGTACACGATGTCCTTCACCTT |
| | CTTCAGGGACTCTCTCATCACGTGGATATTGTGGATATCCAGAAAGAACA |
| | GCTCGGCGTTGTGATAGGCGTCATCAGACTCATATCCTCCTCCTGTTGCC |
| | TTATTTGCCACTGCATTCACAGAAGGCCGGGCATCATAGATTGTCAGCTT |
| | GCTGATCTGCTTATTTGTCTCTCTGATCACATCCAGGTACTTCTCGTCAT |
| | CCTTGTTCCGCTTGCCAGACATTCCCACCAGTGGCTGGGAGCAGCGCACG |
| | ATCACTGTCTTATTCTCTGGGTGGATCCAGCTCAGCACAGGGATTCTGTT |
| | CCGGCTCCGGAAGGTGGCCACTCTCCGCAGGTCATCGTCGCTGGCTCTGT |
| | AGGGCACCACCAGCAGGGCTGGATATGTATCGCACAGCTCGTAGCACTTA |
| | TTGATAAAGGTGATCCGCCAGTGGTGGTTAGGCAGGCCCTGGCGCCTATA |
| | CTCCTCCACGGGGTTGTACACTGTCCAGCCGTCCACATTGAACTTCTCCT |
| | CGTTCAGAAAGGCGAACAGAGGCAGAGAGTGGGCCAGGGGAAAGGCATAT |
| | CTGGTCAGGATCTCGAACATATCTCTCCGGGAGTGGCCCTCCTGCTTCAG |
| | GGCAAACCGCAGATTGCGCATGTCCTTACAGGTGATATCCAGGCCGTAGC |
| | TGTTCTCTCCCCTAGAGGTGGCTCCTCCCATCTTCTCGATTCTGCTGATC |
| | ACGCCCAGAGGCACGTCCAGGATCAGGGAGCTATCTGTCTCCAGGCTCCG |
| | CAGGTACAGTCTGTAATTGGTGATATACACGCGGCCCTTGATTGGGCCGT |
| | TGAAAGGGCAGATGTAGATCACTTCCTTGTCTGTGATCAGTGTCTCTCCA |
| | GGCAGCCTTGGCACTGCCTCTGTCAGATCTCTATTGACCCCGTCCCTTGA |
| | GGTTCTCTTGATGCTCTCGTTTTCCAGGCTATGTGAGTTGTATTTGCTTG |
| | TGCTTGCTGATGCCAT |
| SEQ ID | ATGGCTTCTGCATCAACTTCTAAATATAATTCACACTCCTTGGAGAATGA |
| NO: | GTCTATTAAGAGGACGTCTCGAGATGGAGTCAATCGAGATCTCACTGAGG |
| 168 | CTGTTCCTCGACTTCCAGGAGAAACACTAATCACTGACAAAGAAGTTATT |
| (A5) | TACATATGTCCTTTCAATGGCCCCATTAAGGGAAGAGTTTACATCACAAA |
| (Eurofins | TTATCGTCTTTATTTAAGAAGTTTGGAAACGGATTCTTCTCTAATACTTG |
| codon | ATGTTCCTCTG |
| optimized | GGTGTGATCTCGAGAATTGAAAAAATGGGAGGCGCGACAAGTAGAGGAGA |
| MTM1) | AAATTCCTATGGTCTAGATATTACTTGTAAAGACATGAGAAACCTGAGGT |
| | TCGCTTTGAAACAGGAAGGCCACAGCAGAAGAGATATGTTTGAGATCCTC |
| | ACGAGATACGCGTTTCCCCTGGCTCACAGTCTGCCATTATTTGCATTTTT |
| | AAATGAAGAAAAGTTTAACGTGGATGGATGGACAGTTTACAATCCAGTGG |
| | AAGAATACAGGAGGCAGGGCTTGCCCAATCACCATTGGAGAATAACTTTT |
| | ATTAATAAGTGCTATGAGCTCTGTGACACTTACCCTGCTCTTTTGGTGGT |
| | TCCGTATCGTGCCTCAGATGATGACCTCCGGAGAGTTGCAACTTTTAGGT |
| | CCCGAAATCGAATTCCAGTGCTGTCATGGATTCATCCAGAAAATAAGACG |
| | GTCATTGTGCGTTGCAGTCAGCCTCTTGTCGGTATGAGTGGGAAACGAAA |
| | TAAAGATGATGAGAAATATCTCGATGTTATCAGGGAGACTAATAAACAAA |
| | TTTCTAAACTCACCATTTATGATGCAAGACCCAGCGTAAATGCAGTGGCC |
| | AACAAGGCAACAGGAGGAGGATATGAAAGTGATGATGCATATCATAACGC |
| | CGAACTTTTCTTCTTAGACATTCATAATATTCATGTTATGCGGGAATCTT |
| | TAAAAAAAGTGAAGGACATTGTTTATCCTAATGTAGAAGAATCTCATTGG |
| | TTGTCCAGTTTGGAGTCTACTCATTGGTTAGAACATATCAAGCTCGTTTT |
| | GACAGGAGCCATTCAAGTAGCAGACAAAGTTTCTTCAGGGAAGAGTTCAG |
| | TGCTTGTGCATTGCAGTGACGGATGGGACAGGACTGCTCAGCTGACATCC |
| | TTGGCCATGCTGATGTTGGATAGCTTCTATAGGAGCATTGAAGGGTTCGA |
| | AATACTGGTACAAAAAGAATGGATAAGTTTTGGACATAAATTTGCATCTC |
| | GAATAGGTCATGGTGATAAAAACCACACCGATGCTGACCGTTCTCCTATT |
| | TTTCTCCAGTTTATTGATTGTGTGTGGCAAATGTCAAAACAGTTCCCTAC |
| | AGCTTTTGAATTCAATGAACAATTTTTGATTATAATTTTGGATCATCTGT |
| | ATAGTTGCCGATTTGGTACTTTCTTATTCAACTGTGAATCTGCTCGAGAA |
| | AGACAGAAGGTTACAGAAAGGACTGTTTCTTTATGGTCACTGATAAACAG |
| | TAATAAAGAAAAATTCAAAAACCCATTCTATACTAAAGAAATCAATAGAG |
| | TTTTATATCCAGTTGCAAGTATGCGTCACTTGGAACTCTGGGTGAATTAC |
| | TACATTAGATGGAACCCCAGGATCAAACAACAACAACCAAATCCAGTGGA |
| | ACAACGTTACATGGAACTCTTAGCCTTACGAGATGAATACATAAAACGGC |
| | TTGAGGAACTGCAACTAGCAAACTCTGCAAAACTTTCTGATCCCCCAACT |
| | TCACCTTCCAGTCCTTCTCAAATGATGCCACATGTGCAAACTCACTTTTA |
| | A |
| SEQ ID | ATGGCCTCTGCCAGCACCTCTAAGTACAACAGCCACTCCCTGGAAAATGA |
| NO: | ATCCATCAAAAGGACCAGCAGAGATGGAGTGAACAGAGACCTAACTGAAG |
| 169 | CTGTGCCAAGACTGCCTGGAGAGACCCTGATCACAGACAAGGAAGTGATC |
| (A6) | TACATCTGCCCCTTCAATGGCCCTATCAAGGGAAGGGTGTACATCACCAA |
| (delCpG | CTACAGGCTTTACCTGAGATCCCTGGAGACAGACAGCAGCCTGATCCTGG |
| Genscript | ATGTGCCTCTGGGAGTGATCAGCAGAATAGAGAAGATGGGGGGTGCCACC |
| codon | AGCAGAGGAGAGAACAGCTATGGCCTGGACATCACCTGCAAGGACATGAG |
| optimized | AAACCTGAGATTTGCCCTGAAGCAGGAGGGCCACAGCAGAAGAGACATGT |
| MTM1) | TTGAAATCCTGACCAGGTATGCCTTCCCCCTGGCCCACTCTCTCCCCCTG |
| | TTTGCCTTCCTGAATGAGGAAAAGTTCAATGTTGATGGCTGGACAGTGTA |
| | CAACCCAGTGGAGGAGTACAGAAGACAGGGCCTGCCTAACCACCACTGGA |
| | GGATCACCTTCATCAACAAGTGCTATGAACTGTGTGACACATACCCTGCC |
| | CTGCTGGTGGTGCCTTACAGAGCCTCAGATGATGACCTGAGAAGAGTTGC |
| | CACCTTCAGAAGCAGGAACAGAATCCCTGTACTGAGCTGGATCCACCCTG |
| | AGAATAAGACTGTGATTGTGAGGTGCAGCCAGCCCCTGGTGGGCATGAGT |
| | GGCAAGAGAAACAAAGATGATGAAAAGTACCTGGATGTGATCAGAGAGAC |
| | CAACAAACAGATCAGCAAGCTCACCATCTATGATGCTAGACCCTCTGTTA |
| | ATGCTGTGGCCAACAAGGCCACAGGGGGAGGCTATGAATCTGATGATGCT |
| | TACCACAATGCTGAGCTGTTCTTCCTGGACATCCACAACATCCATGTGAT |
| | GAGAGAATCCCTCAAGAAAGTGAAGGACATTGTGTACCCTAATGTGGAAG |
| | AAAGTCACTGGCTGAGCAGCTTGGAGTCCACCCACTGGCTGGAGCACATC |
| | AAGCTGGTCCTGACAGGAGCCATCCAGGTGGCTGACAAGGTGAGTTCTGG |
| | CAAGTCCTCAGTGCTGGTCCACTGCTCTGATGGCTGGGACAGAACTGCCC |
| | AGCTGACCAGTCTGGCCATGCTGATGCTGGACTCCTTCTACAGAAGCATT |
| | GAAGGCTTTGAAATCCTGGTGCAAAAGGAATGGATCTCTTTTGGCCACAA |
| | GTTTGCCAGCAGAATTGGCCATGGTGACAAAAACCACACAGATGCTGACA |
| | GAAGCCCTATCTTCCTGCAGTTCATTGACTGTGTGTGGCAGATGAGCAAG |
| | CAGTTCCCCACAGCATTTGAGTTCAATGAGCAGTTCCTAATAATCATCCT |
| | GGACCACCTCTACAGCTGCAGATTTGGCACCTTCCTGTTCAACTGTGAGT |
| | CTGCCAGAGAAAGACAGAAGGTGACAGAGAGGACAGTGAGCCTGTGGAGC |
| | CTGATCAACTCCAACAAGGAGAAGTTCAAGAACCCCTTCTACACCAAGGA |
| | AATCAACAGGGTGCTGTACCCTGTGGCTAGCATGAGGCACCTGGAGCTGT |
| | GGGTCAACTACTACATCAGATGGAACCCTAGAATCAAACAGCAACAACCC |
| | AACCCTGTGGAGCAGAGGTACATGGAGTTACTGGCCCTGAGGGATGAGTA |
| | CATCAAGAGACTGGAGGAGCTGCAGCTGGCCAACTCTGCCAAACTGTCTG |
| | ACCCTCCTACCTCCCCCAGCTCCCCCTCTCAGATGATGCCACATGTGCAG |
| | ACCCACTTTTAA |
| SEQ ID | TTAAAAGTGGGTCTGCACGTGAGGCATCATCTGGCTGGGGCTGCTAGGGC |
| NQ 170 | TTGTAGGAGGATCGCTCAGCTTGGCGCTGTTGGCCAGCTGCAGTTCTTCC |
| (A7) | AGTCTCTTGATGTACTCGTCCCGCAGGGCCAGCAGTTCCATGTACCGCTG |
| (GeneArt | TTCCACAGGATTGGGCTGCTGCTGCTTGATTCTGGGGTTCCACCGGATGT |
| codon | AGTAGTTGACCCACAGTTCCAGATGTCTCATGCTGGCCACGGGGTACAGC |
| optimized | ACCCGGTTGATTTCTTTGGTGTAGAAGGGGTTCTTGAATTTCTCTTTGTT |
| MTM1) | GCTGTTGATCAGGGACCACAGAGACACGGTCCGCTCGGTCACTTTCTGCC |
| | GTTCTCTGGCGCTCTCGCAGTTGAACAGGAAGGTGCCGAATCTGCAGCTG |
| | TACAGGTGGTCCAGGATGATGATCAGGAACTGCTCGTTGAACTCGAAGGC |
| | GGTAGGGAACTGCTTGGACATCTGCCACACGCAGTCGATGAACTGCAGGA |
| | AGATGGGGCTTCTATCGGCGTCGGTGTGGTTCTTGTCGCCGTGTCCGATT |
| | CTGCTGGCGAACTTGTGGCCGAAGCTGATCCACTCTTTCTGCACCAGGAT |
| | CTCAAAGCCCTCGATGGATCTGTAGAAGCTGTCCAGCATCAGCATGGCCA |
| | GAGATGTCAGCTGGGCTGTTCTATCCCAGCCGTCGCTACAGTGCACCAGC |
| | ACGCTAGACTTGCCAGAGGACACCTTATCGGCCACCTGGATGGCGCCTGT |
| | CAGCACCAGCTTGATGTGTTCCAGCCAGTGTGTGCTTTCCAGACTGCTCA |
| | GCCAGTGGCTCTCTTCCACATTGGGGTACACGATGTCCTTCACTTTCTTC |
| | AGGCTTTCCCGCATCACGTGGATGTTGTGGATGTCCAGAAAGAACAGCTC |
| | GGCGTTATGATAGGCGTCGTCGCTCTCGTATCCGCCGCCTGTAGCTTTGT |
| | TGGCCACGGCGTTCACGCTAGGTCTGGCGTCGTAGATGGTCAGCTTGCTG |
| | ATCTGCTTGTTTGTCTCGCGGATCACGTCCAGGTACTTCTCGTCGTCCTT |
| | GTTTCTCTTGCCAGACATGCCCACGAGGGGCTGAGAACACCGCACGATCA |
| | CGGTCTTGTTCTCGGGGTGAATCCAGCTCAGCACAGGGATTCTGTTCCGG |
| | CTCCGAAAGGTGGCCACTCTTCTCAGGTCGTCGTCGGAGGCTCTGTAAGG |
| | CACCACCAGCAGTGCGGGGTATGTGTCGCACAGCTCGTAGCACTTGTTGA |
| | TGAAGGTGATCCGCCAGTGGTGATTAGGCAGGCCCTGTCTCCGATACTCT |
| | TCCACGGGGTTGTACACGGTCCAGCCGTCCACGTTGAACTTCTCTTCGTT |
| | CAGGAAGGCGAACAGAGGCAGGGAGTGAGCCAGAGGAAAGGQGTATCTGG |
| | TCAGGATCTCGAACATGTCCCGTCTGCTGTGGCCCTCTTGCTTCAGGGCG |
| | AATCTCAGGTTCCGCATGTCCTTGCATGTGATGTCCAGGCCGTAGCTATT |
| | CTCGCCTCTGGAGGTGGCTCCGCCCATTTTCTCAATCCGGCTGATCACGC |
| | CCAGGGGCACATCCAGGATCAGGCTGCTATCGGTTTCCAGGGACCGCAGG |
| | TACAGCCGGTAGTTGGTGATGTACACGCGGCCCTTGATGGGGCCGTTGAA |
| | GGGGCAGATGTAGATCACTTCTTTGTCGGTGATCAGTGTCTCGCCAGGCA |
| | GTCTAGGCACGGCCTCTGTCAGATCCCTGTTCACGCCATCTCTGCTGGTC |
| | CGCTTGATGCTCTCGTTTTCCAGGCTGTGGCTGTTGTACTTGCTTGTGCT |
| | GGCGCTAGCCAT |
| SEQ ID | GACATTGATTATTGACTAGTTATTAATAGTAATCAATTACGGGGTCATTA |
| NO: | GTTCATAGCCCATATATGGAGTTCCGCGTTACATAACTTACGGTAAATGG |
| 171 | CCCGCCTGGCTGACCGCCCAACGACCCCCGCCCATTGACGTCAATAATGA |
| (B1) | CGTATGTTCCCATAGTAACGCCAATAGGGACTTTCCATTGACGTCAATGG |
| (CMV-IE | GTGGAGTATTTACGGTAAACTGCCCACTTGGCAGTACATCAAGTGTATCA |
| sequence) | TATGCCAAGTACGCCCCCTATTGACGTCAATGACGGTAAATGGCCCGCCT |
| | GGCATTATGCCCAGTACATGACCTTATGGGACTTTCCTACTTGGCAGTAC |
| | ATCTACGTATTAGTCATCGCTATTACCATGGT |
| SEQ ID | TCGAGGTGAGCCCCACGTTCTGCTTCACTCTCCCCATCTCCCCCCCCTCC |
| NQ | CCACCCCCAATTTTGTATTTATTTATTTTTTAATTATTTTGTGCAGCGAT |
| 172 | GGGGGCGGGGGGGGGGGGGGGGCGCGCGCCAGGCGGGGGGGGGGGGGGCG |
| (B2) | AGGGGCGGGGCGGGGCGAGGCGGAGAGGTGCGGCGGCAGCCAATCAGAGC |
| (chicken | GGCGCGCTCCGAAAGTTTCCTTTTATGGCGAGGCGGCGGCGGCGGCGGCC |
| beta | CTATAAAAAGCGAAGCGCGCGGCGGGCG |
| actin | |
| promoter) | |
| SEQ | GACATTGATTATTGACTAGTTATTAATAGTAATCAATTACGGGGTCATTA |
| ID NO: | GTTCATAGCCCATATATGGAGTTCCGCGTTACATAACTTACGGTAAATGG |
| 173 | CCCGCCTGGCTGACCGCCCAACGACCCCCGCCCATTGACGTCAATAATGA |
| (B3) | CGTATGTTCCCATAGTAACGCCAATAGGGACTTTCCATTGACGTCAATGG |
| (CMV- | GTGGAGTATTTACGGTAAACTGCCCACTTGGCAGTACATCAAGTGTATCA |
| IE plus | TATGCCAAGTACGCCCCCTATTGACGTCAATGACGGTAAATGGCCCGCCT |
| chicken | GGCATTATGCCCAGTACATGACCTTATGGGACTTTCCTACTTGGCAGTAC |
| beta | ATCTACGTATTAGTCATCGCTATTACCATGGTTCGAGGTGAGCCCCACGT |
| actin | TCTGCTTCACTCTCCCCATCTCCCCCCCCTCCCCACCCCCAATTTTGTAT |
| promoter) | TTATTTATTTTTTAATTATTITGTGCAGCGATGGGGGCGGGGGGGGGGGG |
| | GGGGCGCGCGCCAGGCGGGGCGGGGCGGGGCGAGGGGGGGGGCGGGGCGA |
| | GGCGGAGAGGTGCGGCGGCAGCCAATCAGAGCGGCGCGCTCCGAAAGTTT |
| | CCTTTTATGGCGAGGCGGCGGCGGCGGCGGCCCTATAAAAAGCGAAGCGC |
| | GCGGCGGGCG |
| SEQ ID | AAAAAATGCTTTCTTCTTTTAATATACTTTTTTGTTTATCTTATTTCTAA |
| NO: | TACTTTCCCTAATCTCTTTCTTTCAGGGCAATAATGATACAATGTATCAT |
| 174 | GCCTCTTTGCACCATTCTAAAGAATAACAGTGATAATTTCTGGGTTAAGG |
| (B4) | CAATAGCAATATTTCTGCATATAAATATTTCTGCATATAAATTGTAACTG |
| (3′ end | ATGTAAGAGGTTTCATATTGCTAATAGCAGCTACAATCCAGCTACCATTC |
| of | TGCTTTTATTTTATGGTTGGGATAAGGCTGGATTATTCTGAGTCCAAGCT |
| human | AGGCCCTTTTGCTAATCATGTTCATACCTCTTATCTTCCTQCCACAG |
| beta | |
| globin | |
| intron | |
| 2) | |
| SEQ ID | GTGCATTGGAACGCGGATTCCCCGTGCCAAGAGTGACGTAAGTACCGCCT |
| NO: | ATAGAGTCTATAGGCCCAC |
| 175 | |
| (B5) | |
| (human | |
| betaberpes | |
| virus | |
| intron) | |
| SEQ ID | GTGCATTGGAACGCGGATTCCCCGTGCCAAGAGTGACGTAAGTACCGCCT |
| NO: | ATAGAGTCTATAGGCCCACAAAAAATGCTTTCTTCTTTTAATATACTTTT |
| 176 | TTGTTTATCTTATTTCTAATACTTTCCCTAATCTCTTTCTTTCAGGGCAA |
| (B6) | TAATGATACAATGTATCATGCCTCTTTGCACCATTCTAAAGAATAACAGT |
| (chimeric | GATAATTTCTGGGTTAAGGCAATAGCAATATTTCTGCATATAAATATTTC |
| intron) | TGCATATAAATTGTAACTGATGTAAGAGGTTTCATATTGCTAATAGCAGC |
| | TACAATCCAGCTACCATTCTGCTTTTATTTTATGGTTGGGATAAGGCTGG |
| | ATTATTCTGAGTCCAAGCTAGGCCCTTTTGCTAATCATGTTCATACCTCT |
| | TATCTTCCTQCCACAG |
| SEQ ID | GATCTTTTTCCCTCTGCCAAAAATTATGGGGACATCATGAAGCCCCTTGA |
| NO: | GCATCTGACTTCTGGCTAATAAAGGAAATTTATTTTCATTGCAATAGTGT |
| 177 | GTTGGAATTTTTTGTGTCTCTCA |
| (D1) | |
| SEQ ID | TTGGCCACTCCCTCTCTGCGCGCTCGCTCGCTCACTGAGGCCGCCCGGGC |
| NQ | AAAGCCCGGGCGTCGGGCGACCTTTGGTCGCCCGGCCTCAGTGAGCGAGC |
| 178 | GAGCGCGCAGAGAGGGAGTGGCCAACTCCATCACTAGGGGTTCCT |
| (D2) | |
| SEQ ID | AGGAACCCCTAGTGATGGAGTTGGCCACTCCCTCTCTGCGCGCTCGCTCG |
| NO: | CTCACTGAGGCCGGGCGACCAAAGGTCGCCCGACGCCCGGGCTTTGCCCG |
| 179 | GGCGGCCTCAGTGAGCGAGCGAGCGCGCAGAGAGGGAGTGGCCAA |
| (D3) | |
| SEQ ID | CTCCTGGGCAACGTGCTGGTCTGTGTGCTGGCCCATCACTTTGGCAAAGA |
| NO: | ATT |
| 180 | |
| (D4) | |
| SEQ ID | TTGGCCACTCCCTCTCTGCGCGCTCGCTCGCTCACTGAGGCCGCCCGGGC |
| NO: | AAAGCCCGGGCGTCGGGCGACCTTTGGTCGCCCGGCCTCAGTGAGCGAGC |
| 181 | GAGCGCGCAGAGAGGGAGTGGCCAACTCCATCACTAGGGGTTCCTTGCGC |
| (E1) | ACAGGTACCCCCCTGCCCCCACAGCTCCTCTCCTGTGCCTTGTTTCCCAG |
| | CCATGQGTTCTCCTCTATAAATACCCGCTCTGGTATTTGGGGTTGGCAGC |
| | TGTTGCTGCCAGGGAGATGGTTGGGTTGACATGCGGCTCCTGACAAAACA |
| | CAAACCCCTGGTGTGTGTGGGCGTGGGTGGTGTGAGTAGGGGGATGAATC |
| | AGGGAGGGGGCGGGGGACCCAGGGGGCAGGAGCCACACAAAGTCTGTGCG |
| | GGGGTGGGAGCGCACATAGCAATTGGAAACTGGCTGCAGACATGCTTGCT |
| | GCCTGCCCTGGCGAAGGATTGGTAGGCTTGCCGTCACAGGACCCCCGCTG |
| | GCTGACTCAGGGGCGCAGGCTCTTGCGGGGGAGCTGGCCTCCCGCCCCCA |
| | CGGCCACGGGCCCTTTCCTGGCAGGACAGCGGGATCTTGCAGCTGTCAGG |
| | GGAGGGGAGGCGGGGGCTGATGTCAGGAGGGATACAAATAGTGCCGACGG |
| | CTGGGGGCCCTGTCTCCCCTCGCCGCATCCACTCTCCGGCCGGCCGCCTG |
| | CCCGCCGCCTCCTCCGTGCGCCCGCCAGCCTCGCCCGCGCCGTCACCGAT |
| | ATCTCAGATCGCCTGGAGACGCCATCCACGCTGTTTTGACCTCCATAGAA |
| | GACACCGGGACCGATCCAGCCTCCGCGGATTCGAATCCCGGCCGGGAACG |
| | GTGCATTGGAACGCGGATTCCCCGTGCCAAGAGTGACGTAAGTACCGCCT |
| | ATAGAGTCTATAGGCCCACAAAAAATGCTTTCTTCTTTTAATATACTTTT |
| | TTGTTTATCTTATTTCTAATACTTTCCCTAATCTCTTTCTTTCAGGGCAA |
| | TAATGATACAATGTATCATGCCTCTTTGCACCATTCTAAAGAATAACAGT |
| | GATAATTTCTGGGTTAAGGCAATAGCAATATTTCTGCATATAAATATTTC |
| | TGCATATAAATTGTAACTGATGTAAGAGGTTTCATATTGCTAATAGCAGC |
| | TACAATCCAGCTACCATTCTGCTTTTATTTTATGGTTGGGATAAGGCTGG |
| | ATTATTCTGAGTCCAAGCTAGGCCCTTTTGCTAATCATGTTCATACCTCT |
| | TATCTTCCTCCCACAGCTCCTGGGCAACGTGCTGGTCTGTGTGCTGGCCC |
| | ATCACTTTGGCAAAGAATTTGAGCGGCCGCCACCATGGCATCAGCAAGCA |
| | CAAGCAAATACAACTCACATAGCCTGGAAAACGAGAGCATCAAGAGAACC |
| | TCAAGGGACGGGGTCAATAGAGATCTGACAGAGGCAGTGCCAAGGCTGCC |
| | TGGAGAGACACTGATCACAGACAAGGAAGTGATCTACATCTGCCCTTTCA |
| | ACGGCCCAATCAAGGGCCGCGTGTATATCACCAATTACAGACTGTACCTG |
| | CGGAGCCTGGAGACAGATAGCTCCCTGATCCTGGACGTGCCTCTGGGCGT |
| | GATCAGCAGAATCGAGAAGATGGGAGGAGCCACCTCTAGGGGAGAGAACA |
| | GCTACGGCCTGGATATCACCTGTAAGGACATGCGCAATCTGCGGTTTGCC |
| | CTGAAGCAGGAGGGCCACTCCCGGAGAGATATGTTCGAGATCCTGACCAG |
| | ATATGCCTTTCCCCTGGCCCACTCTCTGCCTCTGTTCGCCTTTCTGAACG |
| | AGGAGAAGTTCAATGTGGACGGCTGGACAGTGTACAACCCCGTGGAGGAG |
| | TATAGGCGCCAGGGCCTGCCTAACCACCACTGGQGGATCACCTTTATCAA |
| | TAAGTGCTACGAGCTGTGCGATACATATCCAGCCCTGCTGGTGGTGCCCT |
| | ACAGAGCCAGCGACGATGACCTGCGGAGAGTGGCCACCTTCCGGAGCCGG |
| | AACAGAATCCCTGTGCTGAGCTGGATCCACCCAGAGAATAAGACAGTGAT |
| | CGTGCGCTGCTCCCAGCCACTGGTGGGAATGTCTGGCAAGCGGAACAAGG |
| | ATGACGAGAAGTACCTGGATGTGATCAGAGAGACAAATAAGCAGATCAGC |
| | AAGCTGACAATCTATGATGCCCGGCCTTCTGTGAATGCAGTGGCAAATAA |
| | GGCAACAGGAGGAGGATATGAGTCTGATGACGCCTATCACAACGCCGAGC |
| | TGTTCTTTCTGGATATCCACAATATCCACGTGATGAGAGAGTCCCTGAAG |
| | AAGGTGAAGGACATCGTGTACCCAAACGTGGAGGAGTCCCACTGGCTGTC |
| | TAGCCTGGAGTCTACCCACTGGCTGGAGCACATCAAGCTGGTGCTGACAG |
| | GCGCCATCCAGGTGGCCGATAAGGTGTCCTCTGGCAAGAGCTCCGTGCTG |
| | GTGCACTGTTCCGATGGGTGGGACAGAACAGCACAGCTGACAAGCCTGGC |
| | TATGCTGATGCTGGACTCCTTCTATAGATCTATCGAGGGCTTTGAGATCC |
| | TGGTGCAGAAGGAGTGGATCAGCTTCGGCCACAAGTTTGCCTCCAGGATC |
| | GGCCACGGCGATAAGAATCACACCGATGCCGACCGCAGCCCTATCTTCCT |
| | GCAGTTTATCGACTGCGTGTGGCAGATGTCCAAGCAGTTCCCAACCGCCT |
| | TCGAGTTTAACGAGCAGTTTCTGATCATCATCCTGGACCACCTGTACTCC |
| | TGCAGGTTCGGCACATTCCTGTTTAATTGTGAGTCTGCCAGAGAGAGGCA |
| | GAAGGTGACCGAGCGCACAGTGTCTCTGTGGAGCCTGATCAACAGCAATA |
| | AGGAGAAGTTCAAGAACCCCTTCTACACAAAGGAGATCAATCGCGTGCTG |
| | TATCCTGTGGCATCCATGAGACACCTGGAGCTGTGGGTGAACTACTATAT |
| | CCGCTGGAATCCCCGGATCAAGCAGCAGCAGCCCAACCCTGTGGAGCAGC |
| | GGTACATGGAGCTGCTGGCCCTGAGAGATGAGTATATCAAGAGGCTGGAG |
| | GAGCTGCAGCTGGCCAATAGCGCCAAGCTGTCCGACCCACCTACTTCACC |
| | AAGCAGCCCCTCACAGATGATGCCCCACGTCCAGACCCACTTTTAATTAA |
| | GATCTTTTTCCCTCTGCCAAAAATTATGGGGACATCATGAAGCCCCTTGA |
| | GCATCTGACTTCTGGCTAATAAAGGAAATTTATTTTCATTGCAATAGTGT |
| | GTTGGAATTTTTTGTGTCTCTCACTCGAGGCCTAGGAACCCCTAGTGATG |
| | GAGTTGGCCACTCCCTCTCTGCGCGCTCGCTCGCTCACTGAGGCCGGGCG |
| | ACCAAAGGTCGCCCGACGCCCGGGCTTTGCCCGGGCGGCCTCAGTGAGCG |
| | AGCGAGCGCGCAGAGAGGGAGTGGCCAA |
| SEQ ID | TTGGCCACTCCCTCTCTGCGCGCTCGCTCGCTCACTGAGGCCGCCCGGGC |
| NO: | AAAGCCCGGGCGTCGGGCGACCTTTGGTCGCCCGGCCTCAGTGAGCGAGC |
| 182 | GAGCGCGCAGAGAGGGAGTGGCCAACTCCATCACTAGGGGTTCCTTGCGC |
| (E2) | ACAGGTACCGACATTGATTATTGACTAGTTATTAATAGTAATCAATTACG |
| | GGGTCATTAGTTCATAGCCCATATATGGAGTTCCGCGTTACATAACTTAC |
| | GGTAAATGGCCCGCCTGGCTGACCGCCCAACGACCCCCGCCCATTGACGT |
| | CAATAATGACGTATGTTCCCATAGTAACGCCAATAGGGACTTTCCATTGA |
| | CGTCAATGGGTGGAGTATTTACGGTAAACTGCCCACTTGGCAGTACATCA |
| | AGTGTATCATATGCCAAGTACGCCCCCTATTGACGTCAATGACGGTAAAT |
| | GGCCCGCCTGGCATTATGCCCAGTACATGACCTTATGGGACTTTCCTACT |
| | TGGCAGTACATCTACGTATTAGTCATCGCTATTACCATGGTCGAGGTGAG |
| | CCCCACGTTCTGCTTCACTCTCCCCATCTCCCCCCCCTCCCCACCCCCAA |
| | TTTTGTATTTATTTATTTTTTAATTATTTTGTGCAGCGATGGGGGGGGGG |
| | GGGGGGGGGGGGGGCGCGCCAGGCGGGGCGGGGCGGGGCGAGGGGGGGGG |
| | CGGGGCGAGGCGGAGAGGTGCGGCGGCAGCCAATCAGAGCGGCGCGCTCC |
| | GAAAGTTTCCTTTTATGGCGAGGCGGCGGCGGCGGCGGCCCTATAAAAAG |
| | CGAAGCGCGCGGCGGGCGGATATCTCAGATCGCCTGGAGACGCCATCCAC |
| | GCTGTTTTGACCTCCATAGAAGACACCGGGACCGATCCAGCCTCCGCGGA |
| | TTCGAATCCCGGCCGGGAACGGTGCATTGGAACGCGGATTCCCCGTGCCA |
| | AGAGTGACGTAAGTACCGCCTATAGAGTCTATAGGCCCACAAAAAATGCT |
| | TTCTTCTTTTAATATACTTTTTTGTTTATCTTATTTCTAATACTTTCCCT |
| | AATCTCTTTCTTTCAGGGCAATAATGATACAATGTATCATGCCTCTTTGC |
| | ACCATTCTAAAGAATAACAGTGATAATTTCTGGGTTAAGGCAATAGCAAT |
| | ATTTCTGCATATAAATATTTCTGCATATAAATTGTAACTGATGTAAGAGG |
| | TTTCATATTGCTAATAGCAGCTACAATCCAGCTACCATTCTGCTTTTATT |
| | TTATGGTTGGGATAAGGCTGGATTATTCTGAGTCCAAGCTAGGCCCTTTT |
| | GCTAATCATGTTCATACCTCTTATCTTCCTCCCACAGCTCCTGGGCAACG |
| | TGCTGGTCTGTGTGCTGGCCCATCACTTTGGCAAAGAATTTGAGCGGCCG |
| | CCACCATGGCTAGCGCCAGCACAAGCAAGTACAACAGCCACAGCCTGGAA |
| | AACGAGAGCATCAAGCGGACCAGCAGAGATGGCGTGAACAGGGATCTGAC |
| | AGAGGCCGTGCCTAGACTGCCTGGCGAGACACTGATCACCGACAAAGAAG |
| | TGATCTACATCTGCCCCTTCAACGGCCCCATCAAGGGCCGCGTGTACATC |
| | ACCAACTACCGGCTGTACCTGCGGTCCCTGGAAACCGATAGCAGCCTGAT |
| | CCTGGATGTGCCCCTGGGCGTGATCAGCCGGATTGAGAAAATGGGCGGAG |
| | CCACCTCCAGAGGCGAGAATAGCTACGGCCTGGACATCACATGCAAGGAC |
| | ATGCGGAACCTGAGATTCGCCCTGAAGCAAGAGGGCCACAGCAGACGGGA |
| | CATGTTCGAGATCCTGACCAGATACGCCTTTCCTCTGGCTCACTCCCTGC |
| | CTCTGTTCGCCTTCCTGAACGAAGAGAAGTTCAACGTGGACGGCTGGACC |
| | GTGTACAACCCCGTGGAAGAGTATCGGAGACAGGGCCTGCCTAATCACCA |
| | CTGGCGGATCACCTTCATCAACAAGTGCTACGAGCTGTGCGACACATACC |
| | CCGCACTGCTGGTGGTGCCTTACAGAGCCTCCGACGACGACCTGAGAAGA |
| | GTGGCCACCTTTCGGAGCCGGAACAGAATCCCTGTGCTGAGCTGGATTCA |
| | CCCCGAGAACAAGACCGTGATCGTGCGGTGTTCTCAGCCCCTCGTGGGCA |
| | TGTCTGGCAAGAGAAACAAGGACGACGAGAAGTACCTGGACGTGATCCGC |
| | GAGACAAACAAGCAGATCAGCAAGCTGACCATCTACGACGCCAGACCTAG |
| | CGTGAACGCCGTGGCCAACAAAGCTACAGGCGGCGGATACGAGAGCGACG |
| | ACGCCTATCATAACGCCGAGCTGTTCTTTCTGGACATCCACAACATCCAC |
| | GTGATGCGGGAAAGCCTGAAGAAAGTGAAGGACATCGTGTACCCCAATGT |
| | GGAAGAGAGCCACTGGCTGAGCAGTCTGGAAAGCACACACTGGCTGGAAC |
| | ACATCAAGCTGGTGCTGACAGGCGCCATCCAGGTGGCCGATAAGGTGTCC |
| | TCTGGCAAGTCTAGCGTGCTGGTGCACTGTAGCGACGGCTGGGATAGAAC |
| | AGCCCAGCTGACATCTCTGGCCATGCTGATGCTGGACAGCTTCTACAGAT |
| | CCATCGAGGGCTTTGAGATCCTGGTGCAGAAAGAGTGGATCAGCTTCGGC |
| | CACAAGTTCGCCAGCAGAATCGGACACGGCGACAAGAACCACACCGACGC |
| | CGATAGAAGCCCCATCTTCCTGCAGTTCATCGACTGCGTGTGGCAGATGT |
| | CCAAGCAGTTCCCTACCGCCTTCGAGTTCAACGAGCAGTTCCTGATCATC |
| | ATCCTGGACCACCTGTACAGCTGCAGATTCGGCACCTTCCTGTTCAACTG |
| | CGAGAGCGCCAGAGAACGGCAGAAAGTGACCGAGCGGACCGTGTCTCTGT |
| | GGTCCCTGATCAACAGCAACAAAGAGAAATTCAAGAACCCCTTCTACACC |
| | AAAGAAATCAACCGGGTGCTGTACCCCGTGGCCAGCATGAGACATCTGGA |
| | ACTGTGGGTCAACTACTACATCCGGTGGAACCCCAGAATCAAGCAGCAGC |
| | AGCCCAATCCTGTGGAACAGCGGTACATGGAACTGCTGGCCCTGCGGGAC |
| | GAGTACATCAAGAGACTGGAAGAACTGCAGCTGGCCAACAGCGCCAAGCT |
| | GAGCGATCCTCCTACAAGCCCTAGCAGCCCCAGCCAGATGATGCCTCACG |
| | TGCAGACCCACTTTTAATTAAGATCTTTTTCCCTCTGCCAAAAATTATGG |
| | GGACATCATGAAGCCCCTTGAGCATCTGACTTCTGGCTAATAAAGGAAAT |
| | TTATTTTCATTGCAATAGTGTGTTGGAATTTTTTGTGTCTCTCACTCGAG |
| | GCCTAGGAACCCCTAGTGATGGAGTTGGCCACTCCCTCTCTGCGCGCTCG |
| | CTCGCTCACTGAGGCCGGGCGACCAAAGGTCGCCCGACGCCCGGGCTTTG |
| | CCCGGGCGGCCTCAGTGAGCGAGCGAGCGCGCAGAGAGGGAGTGGCCAA |
| SEQ ID | TTGGCCACTCCCTCTCTGCGCGCTCGCTCGCTCACTGAGGCCGCCCGGGC |
| NO: | AAAGCCCGGGCGTCGGGCGACCTTTGGTCGCCCGGCCTCAGTGAGCGAGC |
| 183 | GAGCGCGCAGAGAGGGAGTGGCCAACTCCATCACTAGGGGTTCCTTGCGC |
| (E3) | ACAGGTACCCCCCTGCCCCCACAGCTCCTCTCCTGTGCCTTGTTTCCCAG |
| | CCATGCGTTCTCCTCTATAAATACCCGCTCTGGTATTTGGGGTTGGCAGC |
| | TGTTGCTGCCAGGGAGATGGTTGGGTTGACATGCGGCTCCTGACAAAACA |
| | CAAACCCCTGGTGTGTGTGGGCGTGGGTGGTGTGAGTAGGGGGATGAATC |
| | AGGGGGGGGCGGGGGACCCAGGGGGCAGGAGCCACACAAAGTCTGTGCGG |
| | GGGTGGGAGCGCACATAGCAATTGGAAACTGGCTGCAGACATGCTTGCTG |
| | CCTGCCCTGGCGAAGGATTGGTAGGCTTGCCGTCACAGGACCCCCGCTGG |
| | CTGACTCAGGGGCGCAGGCTCTTGCGGGGGAGCTGGCCTCCCGCCCCCAC |
| | GGCCACGGGCCCTTTCCTGGCAGGACAGCGGGATCTTGCAGCTGTCAGGG |
| | GAGGGGAGGCGGGGGCTGATGTCAGGAGGGATACAAATAGTGCCGACGGC |
| | TGGGGGCCCTGTCTCCCCTCGCCGCATCCACTCTCCGGCCGGCCGCCTGC |
| | CCGCCGCCTCCTCCGTGCGCCCGCCAGCCTCGCCCGCGCCGTCACCGATA |
| | TCTCAGATCGCCTGGAGACGCCATCCACGCTGTTTTGACCTCCATAGAAG |
| | ACACCGGGACCGATCCAGCCTCCGCGGATTCGAATCCCGGCCGGGAACGG |
| | TGCATTGGAACGCGGATTCCCCGTGCCAAGAGTGACGTAAGTACCGCCTA |
| | TAGAGTCTATAGGCCCACAAAAAATGCTTTCTTCTTTTAATATACTTTTT |
| | TGTTTATCTTATTTCTAATACTTTCCCTAATCTCTTTCTTTCAGGGCAAT |
| | AATGATACAATGTATCATGCCTCTTTGCACCATTCTAAAGAATAACAGTG |
| | ATAATTTCTGGGTTAAGGCAATAGCAATATTTCTGCATATAAATATTTCT |
| | GCATATAAATTGTAACTGATGTAAGAGGTTTCATATTGCTAATAGCAGCT |
| | ACAATCCAGCTACCATTCTGCTTTTATTTTATGGTTGGGATAAGGCTGGA |
| | TTATTCTGAGTCCAAGCTAGGCCCTTTTGCTAATCATGTTCATACCTCTT |
| | ATCTTCCTCCCACAGCTCCTGGGCAACGTGCTGGTCTGTGTGCTGGCCCA |
| | TCACTTTGGCAAAGAATTTGAGCGGCCGCCACCATGGCTTCTGCATCAAC |
| | TTCTAAATATAATTCACACTCCTTGGAGAATGAGTCTATTAAGAGGACGT |
| | CTCGAGATGGAGTCAATCGAGATCTCACTGAGGCTGTTCCTCGACTTCCA |
| | GGAGAAACACTAATCACTGACAAAGAAGTTATTTACATATGTCCTTTCAA |
| | TGGCCCCATTAAGGGAAGAGTTTACATCACAAATTATCGTCTTTATTTAA |
| | GAAGTTTGGAAACGGATTCTTCTCTAATACTTGATGTTCCTCTGGGTGTG |
| | ATCTCGAGAATTGAAAAAATGGGAGGCGCGACAAGTAGAGGAGAAAATTC |
| | CTATGGTCTAGATATTACTTGTAAAGACATGAGAAACCTGAGGTTCGCTT |
| | TGAAACAGGAAGGCCACAGCAGAAGAGATATGTTTGAGATCCTCACGAGA |
| | TACGCGTTTCCCCTGGCTCACAGTCTGCCATTATTTGCATTTTTAAATGA |
| | AGAAAAGTTTAACGTGGATGGATGGACAGTTTACAATCCAGTGGAAGAAT |
| | ACAGGAGGCAGGGCTTGCCCAATCACCATTGGAGAATAACTTTTATTAAT |
| | AAGTGCTATGAGCTCTGTGACACTTACCCTGCTCTTTTGGTGGTTCCGTA |
| | TCGTGCCTCAGATGATGACCTCCGGAGAGTTGCAACTTTTAGGTCCCGAA |
| | ATCGAATTCCAGTGCTGTCATGGATTCATCCAGAAAATAAGACGGTCATT |
| | GTGCGTTGCAGTCAGCCTCTTGTCGGTATGAGTGGGAAACGAAATAAAGA |
| | TGATGAGAAATATCTCGATGTTATCAGGGAGACTAATAAACAAATTTCTA |
| | AACTCACCATTTATGATGCAAGACCCAGCGTAAATGCAGTGGCCAACAAG |
| | GCAACAGGAGGAGGATATGAAAGTGATGATGCATATCATAACGCCGAACT |
| | TTTCTTCTTAGACATTCATAATATTCATGTTATGCGGGAATCTTTAAAAA |
| | AAGTGAAGGACATTGTTTATCCTAATGTAGAAGAATCTCATTGGTTGTCC |
| | AGTTTGGAGTCTACTCATTGGTTAGAACATATCAAGCTCGTTTTGACAGG |
| | AGCCATTCAAGTAGCAGACAAAGTTTCTTCAGGGAAGAGTTCAGTGCTTG |
| | TGCATTGCAGTGACGGATGGGACAGGACTGCTCAGCTGACATCCTTGGCC |
| | ATGCTGATGTTGGATAGCTTCTATAGGAGCATTGAAGGGTTCGAAATACT |
| | GGTACAAAAAGAATGGATAAGTTTTGGACATAAATTTGCATCTCGAATAG |
| | GTCATGGTGATAAAAACCACACCGATGCTGACCGTTCTCCTATTTTTCTC |
| | CAGTTTATTGATTGTGTGTGGCAAATGTCAAAACAGTTCCCTACAGCTTT |
| | TGAATTCAATGAACAATTTTTGATTATAATTTTGGATCATCTGTATAGTT |
| | GCCGATTTGGTACTTTCTTATTCAACTGTGAATCTGCTCGAGAAAGACAG |
| | AAGGTTACAGAAAGGACTGTTTCTTTATGGTCACTGATAAACAGTAATAA |
| | AGAAAAATTCAAAAACCCATTCTATACTAAAGAAATCAATAGAGTTTTAT |
| | ATCCAGTTGCAAGTATGCGTCACTTGGAACTCTGGGTGAATTACTACATT |
| | AGATGGAACCCCAGGATCAAACAACAACAACCAAATCCAGTGGAACAACG |
| | TTACATGGAACTCTTAGCCTTACGAGATGAATACATAAAACGGCTTGAGG |
| | AACTGCAACTAGCAAACTCTGCAAAACTTTCTGATCCCCCAACTTCACCT |
| | TCCAGTCCTTCTCAAATGATGCCACATGTGCAAACTCACTTTTAATTAAG |
| | ATCTTTTTCCCTCTGCCAAAAATTATGGGGACATCATGAAGCCCCTTGAG |
| | CATCTGACTTCTGGCTAATAAAGGAAATTTATTTTCATTGCAATAGTGTG |
| | TTGGAATTTTTTGTGTCTCTCACTCGAGGCCTAGGAACCCCTAGTGATGG |
| | AGTTGGCCACTCCCTCTCTGCGCGCTCGCTCGCTCACTGAGGCCGGGCGA |
| | CCAAAGGTCGCCCGACGCCCGGGCTTTGCCCGGGCGGCCTCAGTGAGCGA |
| | GCGAGCGCGCAGAGAGGGAGTGGCCAA |
| SEQ ID | TTGGCCACTCCCTCTCTGCGCGCTCGCTCGCTCACTGAGGCCGCCCGGGC |
| NO: | AAAGCCCGGGCGTCGGGCGACCTTTGGTCGCCCGGCCTCAGTGAGCGAGC |
| 184 | GAGCGCGCAGAGAGGGAGTGGCCAACTCCATCACTAGGGGTTCCTTGCGC |
| (E4) | ACAGGTACCCCCCTGCCCCCACAGCTCCTCTCCTGTGCCTTGTTTCCCAG |
| | CCATGCGTTCTCCTCTATAAATACCCGCTCTGGTATTTGGGGTTGGCAGC |
| | TGTTGCTGCCAGGGAGATGGTTGGGTTGACATGCGGCTCCTGACAAAACA |
| | CAAACCCCTGGTGTGTGTGGGCGTGGGTGGTGTGAGTAGGGGGATGAATC |
| | AGGGAGGGGGCGGGGGACCCAGGGGGCAGGAGCCACACAAAGTCTGTGCG |
| | GGGGGGGAGCGCACATAGCAATTGGAAACTGGCTGCAGACATGCTTGCTG |
| | CCTGCCCTGGCGAAGGATTGGTAGGCTTGCCGTCACAGGACCCCCGCTGG |
| | CTGACTCAGGGGCGCAGGCTCTTGCGGGGGAGCTGGCCTCCCGCCCCCAC |
| | GGCCACGGGCCCTTTCCTGGCAGGACAGCGGGATCTTGCAGCTGTCAGGG |
| | GAGGGGAGGCGGGGGCTGATGTCAGGAGGGATACAAATAGTGCCGACGGC |
| | TGGGGGCCCTGTCTCCCCTCGCCGCATCCACTCTCCGGCCGGCCGCCTGC |
| | CCGCCGCCTCCTCCGTGCGCCCGCCAGCCTCGCCCGCGCCGTCACCGATA |
| | TCTCAGATCGCCTGGAGACGCCATCCACGCTGTTTTGACCTCCATAGAAG |
| | ACACCGGGACCGATCCAGCCTCCGCGGATTCGAATCCCGGCCGGGAACGG |
| | TGCATTGGAACGCGGATTCCCCGTGCCAAGAGTGACGTAAGTACCGCCTA |
| | TAGAGTCTATAGGCCCACAAAAAATGCTTTCTTCTTTTAATATACTTTTT |
| | TGTTTATCTTATTTCTAATACTTTCCCTAATCTCTTTCTTTCAGGGCAAT |
| | AATGATACAATGTATCATGCCTCTTTGCACCATTCTAAAGAATAACAGTG |
| | ATAATTTCTGGGTTAAGGCAATAGCAATATTTCTGCATATAAATATTTCT |
| | GCATATAAATTGTAACTGATGTAAGAGGTTTCATATTGCTAATAGCAGCT |
| | ACAATCCAGCTACCATTCTGCTTTTATITTATGGTTGGGATAAGGCTGGA |
| | TTATTCTGAGTCCAAGCTAGGCCCTTTTGCTAATCATGTTCATACCTCTT |
| | ATCTTCCTCCCACAGCTCCTGGGCAACGTGCTGGTCTGTGTGCTGGCCCA |
| | TCACTTTGGCAAAGAATTTGAGCGGCCGCCACCATGGCTTCTGCATCAAC |
| | TTCTAAATATAATTCACACTCCTTGGAGAATGAGTCTATTAAGAGGACGT |
| | CTCGAGATGGAGTCAATCGAGATCTCACTGAGGCTGTTCCTCGACTTCCA |
| | GGAGAAACACTAATCACTGACAAAGAAGTTATTTACATATGTCCTTTCAA |
| | TGGCCCCATTAAGGGAAGAGTTTACATCACAAATTATCGTCTTTATTTAA |
| | GAAGTTTGGAAACGGATTCTTCTCTAATACTTGATGTTCCTCTGGGTGTG |
| | ATCTCGAGAATTGAAAAAATGGGAGGCGCGACAAGTAGAGGAGAAAATTC |
| | CTATGGTCTAGATATTACTTGTAAAGACATGAGAAACCTGAGGTTCGCTT |
| | TGAAACAGGAAGGCCACAGCAGAAGAGATATGTTTGAGATCCTCACGAGA |
| | TACGCGTTTCCCCTGGCTCACAGTCTGCCATTATTTGCATTTTTAAATGA |
| | AGAAAAGITTAACGTGGATGGATGGACAGTTTACAATCCAGTGGAAGAAT |
| | ACAGGAGGCAGGGCTTGCCCAATCACCATTGGAGAATAACTTTTATTAAT |
| | AAGTGCTATGAGCTCTGTGACACTTACCCTGCTCTTTTGGTGGTTCCGTA |
| | TCGTGCCTCAGATGATGACCTCCGGAGAGTTGCAACTTTTAGGTCCCGAA |
| | ATCGAATTCCAGTGCTGTCATGGATTCATCCAGAAAATAAGACGGTCATT |
| | GTGCGTTGCAGTCAGCCTCTTGTCGGTATGAGTGGGAAACGAAATAAAGA |
| | TGATGAGAAATATCTCGATGTTATCAGGGAGACTAATAAACAAATTTCTA |
| | AACTCACCATTTATGATGCAAGACCCAGCGTAAATGCAGTGGCCAACAAG |
| | GCAACAGGAGGAGGATATGAAAGTGATGATGCATATCATAACGCCGAACT |
| | TTTCTTCTTAGACATTCATAATATTCATGTTATGCGGGAATCTTTAAAAA |
| | AAGTGAAGGACATTGTTTATCCTAATGTAGAAGAATCTCATTGGTTGTCC |
| | AGTTTGGAGTCTACTCATTGGTTAGAACATATCAAGCTCGTTTTGACAGG |
| | AGCCATTCAAGTAGCAGACAAAGTTTCTTCAGGGAAGAGTTCAGTGCTTG |
| | TGCATTGCAGTGACGGATGGGACAGGACTGCTCAGCTGACATCCTTGGCC |
| | ATGCTGATGTTGGATAGCTTCTATAGGAGCATTGAAGGGTTCGAAATACT |
| | GGTACAAAAAGAATGGATAAGTTTTGGACATAAATTTGCATCTCGAATAG |
| | GTCATGGTGATAAAAACCACACCGATGCTGACCGTTCTCCTATTTTTCTC |
| | CAGTTTATTGATTGTGTGTGGCAAATGTCAAAACAGTTCCCTACAGCTTT |
| | TGAATTCAATGAACAATTTTTGATTATAATTTTGGATCATCTGTATAGTT |
| | GCCGATTTGGTACTTTCTTATTCAACTGTGAATCTGCTCGAGAAAGACAG |
| | AAGGTTACAGAAAGGACTGTTTCTTTATGGTCACTGATAAACAGTAATAA |
| | AGAAAAATTCAAAAACCCCTTCTATACTAAAGAAATCAATCGAGTTTTAT |
| | ATCCAGTTGCCAGTATGCGTCACTTGGAACTCTGGGTGAATTACTACATT |
| | AGATGGAACCCCAGGATCAAGCAACAACAGCCGAATCCAGTGGAGCAGCG |
| | TTACATGGAGCTCTTAGCCTTACGCGACGAATACATAAAGCGGCTTGAGG |
| | AACTGCAGCTCGCCAACTCTGCCAAGCTTTCTGATCCCCCAACTTCACCT |
| | TCCAGTCCTTCGCAAATGATGCCCCATGTGCAAACTCACTTTTAATTAAG |
| | ATCTTTTTCCCTCTGCCAAAAATTATGGGGACATCATGAAGCCCCTTGAG |
| | CATCTGACTTCTGGCTAATAAAGGAAATTTATTTTCATTGCAATAGTGTG |
| | TTGGAATTTTTTGTGTCTCTCACTCGAGGCCTAGGAACCCCTAGTGATGG |
| | AGTTGGCCACTCCCTCTCTGCGCGCTCGCTCGCTCACTGAGGCCGGGCGA |
| | CCAAAGGTCGCCCGACGCCCGGGCTTTGCCCGGGCGGCCTCAGTGAGCGA |
| | GCGAGCGCGCAGAGAGGGAGTGGCCAA |
| SEQ ID | TTGGCCACTCCCTCTCTGCGCGCTCGCTCGCTCACTGAGGCCGCCCGGGC |
| NO: | AAAGCCCGGGCGTCGGGCGACCTTTGGTCGCCCGGCCTCAGTGAGCGAGC |
| 185 | GAGCGCGCAGAGAGGGAGTGGCCAACTCCATCACTAGGGGTTCCTTGCGC |
| (ES) | ACAGGTACCCCCCTGCCCCCACAGCTCCTCTCCTGTGCCTTGTTTCCCAG |
| | CCATGCGTTCTCCTCTATAAATACCCGCTCTGGTATTTGGGGTTGGCAGC |
| | TGTTGCTGCCAGGGAGATGGTTGGGTTGACATGCGGCTCCTGACAAAACA |
| | CAAACCCCTGGTGTGTGTGGGCGTGGGTGGTGTGAGTAGGGGGATGAATC |
| | AGGGAGGGGGCGGGGGACCCAGGGGGCAGGAGCCACACAAAGTCTGTGCG |
| | GGGGGGGAGCGCACATAGCAATTGGAAACTGGCTGCAGACATGCTTGCTG |
| | CCTGCCCTGGCGAAGGATTGGTAGGCTTGCCGTCACAGGACCCCCGCTGG |
| | CTGACTCAGGGGCGCAGGCTCTTGCGGGGGAGCTGGCCTCCCGCCCCCAC |
| | GGCCACGGGCCCTTTCCTGGCAGGACAGCGGGATCTTGCAGCTGTCAGGG |
| | GAGGGGAGGCGGGGGCTGATGTCAGGAGGGATACAAATAGTGCCGACGGC |
| | TGGGGGCCCTGTCTCCCCTCGCCGCATCCACTCTCCGGCCGGCCGCCTGC |
| | CCGCCGCCTCCTCCGTGCGCCCGCCAGCCTCGCCCGCGCCGTCACCGATA |
| | TCTCAGATCGCCTGGAGACGCCATCCACGCTGTTTTGACCTCCATAGAAG |
| | ACACCGGGACCGATCCAGCCTCCGCGGATTQGAATCCCGGCCGGGAACGG |
| | TGCATTGGAACGCGGATTCCCCGTGCCAAGAGTGACGTAAGTACCGCCTA |
| | TAGAGTCTATAGGCCCACAAAAAATGCTTTCTTCTTTTAATATACTTTTT |
| | TGTTTATCTTATTTCTAATACTTTCCCTAATCTCTTTCTTTCAGGGCAAT |
| | AATGATACAATGTATCATGCCTCTTTGCACCATTCTAAAGAATAACAGTG |
| | ATAATTTCTGGGTTAAGGCAATAGCAATATTTCTGCATATAAATATTTCT |
| | GCATATAAATTGTAACTGATGTAAGAGGTTTCATATTGCTAATAGCAGCT |
| | ACAATCCAGCTACCATTCTGCTTTTATTTTATGGTTGGGATAAGGCTGGA |
| | TTATTCTGAGTCCAAGCTAGGCCCTTTTGCTAATCATGTTCATACCTCTT |
| | ATCTTCCTCCCACAGCTCCTGGGCAACGTGCTGGTCTGTGTGCTGGCCCA |
| | TCACTTTGGCAAAGAATTTGAGCGGCCGCCACCATGGCCTCTGCCAGCAC |
| | CTCTAAGTACAACAGCCACTCCCTGGAAAATGAATCCATCAAAAGGACCA |
| | GCAGAGATGGAGTGAACAGAGACCTAACTGAAGCTGTGCCAAGACTGCCT |
| | GGAGAGACCCTGATCACAGACAAGGAAGTGATCTACATCTGCCCCTTCAA |
| | TGGCCCTATCAAGGGAAGGGTGTACATCACCAACTACAGGCTTTACCTGA |
| | GATCCCTGGAGACAGACAGCAGCCTGATCCTGGATGTGCCTCTGGGAGTG |
| | ATCAGCAGAATAGAGAAGATGGGGGGTGCCACCAGCAGAGGAGAGAACAG |
| | CTATGGCCTGGACATCACCTGCAAGGACATGAGAAACCTGAGATTTGCCC |
| | TGAAGCAGGAGGGCCACAGCAGAAGAGACATGTTTGAAATCCTGACCAGG |
| | TATGCCTTCCCCCTGGCCCACTCTCTCCCCCTGTTTGCCTTCCTGAATGA |
| | GGAAAAGTTCAATGTTGATGGCTGGACAGTGTACAACCCAGTGGAGGAGT |
| | ACAGAAGACAGGGCCTGCCTAACCACCACTGGAGGATCACCTTCATCAAC |
| | AAGTGCTATGAACTGTGTGACACATACCCTGCCCTGCTGGTGGTGCCTTA |
| | CAGAGCCTCAGATGATGACCTGAGAAGAGTTGCCACCTTCAGAAGCAGGA |
| | ACAGAATCCCTGTACTGAGCTGGATCCACCCTGAGAATAAGACTGTGATT |
| | GTGAGGTGCAGCCAGCCCCTGGTGGGCATGAGTGGCAAGAGAAACAAAGA |
| | TGATGAAAAGTACCTGGATGTGATCAGAGAGACCAACAAACAGATCAGCA |
| | AGCTCACCATCTATGATGCTAGACCCTCTGTTAATGCTGTGGCCAACAAG |
| | GCCACAGGGGGAGGCTATGAATCTGATGATGCTTACCACAATGCTGAGCT |
| | GTTCTTCCTGGACATCCACAACATCCATGTGATGAGAGAATCCCTCAAGA |
| | AAGTGAAGGACATTGTGTACCCTAATGTGGAAGAAAGTCACTGGCTGAGC |
| | AGCTTGGAGTCCACCCACTGGCTGGAGCACATCAAGCTGGTCCTGACAGG |
| | AGCCATCCAGGTGGCTGACAAGGTGAGTTCTGGCAAGTCCTCAGTGCTGG |
| | TCCACTGCTCTGATGGCTGGGACAGAACTGCCCAGCTGACCAGTCTGGCC |
| | ATGCTGATGCTGGACTCCTTCTACAGAAGCATTGAAGGCTTTGAAATCCT |
| | GGTGCAAAAGGAATGGATCTCTTTTGGCCACAAGTTTGCCAGCAGAATTG |
| | GCCATGGTGACAAAAACCACACAGATGCTGACAGAAGCCCTATCTTCCTG |
| | CAGTTCATTGACTGTGTGTGGCAGATGAGCAAGCAGTTCCCCACAGCATT |
| | TGAGTTCAATGAGCAGTTCCTAATAATCATCCTGGACCACCTCTACAGCT |
| | GCAGATTTGGCACCTTCCTGTTCAACTGTGAGTCTGCCAGAGAAAGACAG |
| | AAGGTGACAGAGAGGACAGTGAGCCTGTGGAGCCTGATCAACTCCAACAA |
| | GGAGAAGTTCAAGAACCCCTTCTACACCAAGGAAATCAACAGGGTGCTGT |
| | ACCCTGTGGCTAGCATGAGGCACCTGGAGCTGTGGGTCAACTACTACATC |
| | AGATGGAACCCTAGAATCAAACAGCAACAACCCAACCCTGTGGAGCAGAG |
| | GTACATGGAGTTACTGGCCCTGAGGGATGAGTACATCAAGAGACTGGAGG |
| | AGCTGCAGCTGGCCAACTCTGCCAAACTGTCTGACCCTCCTACCTCCCCC |
| | AGCTCCCCCTCTCAGATGATGCCACATGTGCAGACCCACTTTTAATTAAG |
| | ATCTTTTTCCCTCTGCCAAAAATTATGGGGACATCATGAAGCCCCTTGAG |
| | CATCTGACTTCTGGCTAATAAAGGAAATTTATTTTCATTGCAATAGTGTG |
| | TTGGAATTTTTTGTGTCTCTCACTCGAGGCCTAGGAACCCCTAGTGATGG |
| | AGTTGGCCACTCCCTCTCTGCGCGCTCGCTCGCTCACTGAGGCCGGGCGA |
| | CCAAAGGTCGCCCGACGCCCGGGCTTTGCCCGGGCGGCCTCAGTGAGCGA |
| | GCGAGCGCGCAGAGAGGGAGTGGCCAA |
| SEQ ID | TTGGCCACTCCCTCTCTGCGCGCTCGCTCGCTCACTGAGGCCGCCCGGGC |
| NO: | AAAGCCCGGGCGTCGGGCGACCTTTGGTCGCCCGGCCTCAGTGAGCGAGC |
| 186 | GAGCGCGCAGAGAGGGAGTGGCCAACTCCATCACTAGGGGTTCCTTGCGC |
| (E6) | ACAGGTACCGACATTGATTATTGACTAGTTATTAATAGTAATCAATTACG |
| | GGGTCATTAGTTCATAGCCCATATATGGAGTTCCGCGTTACATAACTTAC |
| | GGTAAATGGCCCGCCTGGCTGACCGCCCAACGACCCCCGCCCATTGACGT |
| | CAATAATGACGTATGTTCCCATAGTAACGCCAATAGGGACTTTCCATTGA |
| | CGTCAATGGGTGGAGTATTTACGGTAAACTGCCCACTTGGCAGTACATCA |
| | AGTGTATCATATGCCAAGTACGCCCCCTATTGACGTCAATGACGGTAAAT |
| | GGCCCGCCTGGCATTATGCCCAGTACATGACCTTATGGGACTTTCCTACT |
| | TGGCAGTACATCTACGTATTAGTCATCGCTATTACCATGGTCGAGGTGAG |
| | CCCCACGTTCTGCTTCACTCTCCCCATCTCCCCCCCCTCCCCACCCCCAA |
| | TTTTGTATTTATTTATTTTTTAATTATTTTGTGCAGCGATGGGGGGGGGG |
| | GGGGGGGGGGGGGGCGCGCCAGGCGGGGCGGGGGGGGGCGAGGGGGGGGG |
| | GGGGCGAGGCGGAGAGGTGCGGCGGCAGCCAATCAGAGCGGCGCGCTCCG |
| | AAAGTTTCCTTTTATGGCGAGGCGGCGGCGGCGGCGGCCCTATAAAAAGC |
| | GAAGCGCGCGGCGGGCGGATATCTCAGATCGCCTGGAGACGCCATCCACG |
| | CTGTTTTGACCTCCATAGAAGACACCGGGACCGATCCAGCCTCCGCGGAT |
| | TCGAATCCCGGCCGGGAACGGTGCATTGGAACGCGGATTCCCCGTGCCAA |
| | GAGTGACGTAAGTACCGCCTATAGAGTCTATAGGCCCACAAAAAATGCTT |
| | TCTTCTTTTAATATACTTTTTTGTTTATCTTATTTCTAATACTTTCCCTA |
| | ATCTCTTTCTTTCAGGGCAATAATGATACAATGTATCATGCCTCTTTGCA |
| | CCATTCTAAAGAATAACAGTGATAATTTCTGGGTTAAGGCAATAGCAATA |
| | TTTCTGCATATAAATATTTCTGCATATAAATTGTAACTGATGTAAGAGGT |
| | TTCATATTGCTAATAGCAGCTACAATCCAGCTACCATTCTGCTTTTATTT |
| | TATGGTTGGGATAAGGCTGGATTATTCTGAGTCCAAGCTAGGCCCTTTTG |
| | CTAATCATGTTCATACCTCTTATCTTCCTCCCACAGCTCCTGGGCAACGT |
| | GCTGGTCTGTGTGCTGGCCCATCACTTTGGCAAAGAATTTGAGCGGCCGC |
| | CACCATGGCATCAGCAAGCACAAGCAAATACAACTCACATAGCCTGGAAA |
| | ACGAGAGCATCAAGAGAACCTCAAGGGACGGGGTCAATAGAGATCTGACA |
| | GAGGCAGTGCCAAGGCTGCCTGGAGAGACACTGATCACAGACAAGGAAGT |
| | GATCTACATCTGCCCTTTCAACGGCCCAATCAAGGGCCGCGTGTATATCA |
| | CCAATTACAGACTGTACCTGCGGAGCCTGGAGACAGATAGCTCCCTGATC |
| | CTGGACGTGCCTCTGGGCGTGATCAGCAGAATCGAGAAGATGGGAGGAGC |
| | CACCTCTAGGGGAGAGAACAGCTACGGCCTGGATATCACCTGTAAGGACA |
| | TGCGCAATCTGCGGTTTGCCCTGAAGCAGGAGGGCCACTCCCGGAGAGAT |
| | ATGTTCGAGATCCTGACCAGATATGCCTTTCCCCTGGCCCACTCTCTGCC |
| | TCTGTTCGCCTTTCTGAACGAGGAGAAGTTCAATGTGGACGGCTGGACAG |
| | TGTACAACCCCGTGGAGGAGTATAGGCGCCAGGGCCTGCCTAACCACCAC |
| | TGGCGGATCACCTTTATCAATAAGTGCTACGAGCTGTGCGATACATATCC |
| | AGCCCTGCTGGTGGTGCCCTACAGAGCCAGCGACGATGACCTGCGGAGAG |
| | TGGCCACCTTCCGGAGCCGGAACAGAATCCCTGTGCTGAGCTGGATCCAC |
| | CCAGAGAATAAGACAGTGATCGTGCGCTGCTCCCAGCCACTGGTGGGAAT |
| | GTCTGGCAAGCGGAACAAGGATGACGAGAAGTACCTGGATGTGATCAGAG |
| | AGACAAATAAGCAGATCAGCAAGCTGACAATCTATGATGCCCGGCCTTCT |
| | GTGAATGCAGTGGCAAATAAGGCAACAGGAGGAGGATATGAGTCTGATGA |
| | CGCCTATCACAACGCCGAGCTGTTCTTTCTGGATATCCACAATATCCACG |
| | TGATGAGAGAGTCCCTGAAGAAGGTGAAGGACATCGTGTACCCAAACGTG |
| | GAGGAGTCCCACTGGCTGTCTAGCCTGGAGTCTACCCACTGGCTGGAGCA |
| | CATCAAGCTGGTGCTGACAGGCGCCATCCAGGTGGCCGATAAGGTGTCCT |
| | CTGGCAAGAGCTCCGTGCTGGTGCACTGTTCCGATGGGTGGGACAGAACA |
| | GCACAGCTGACAAGCCTGGCTATGCTGATGCTGGACTCCTTCTATAGATC |
| | TATCGAGGGCTTTGAGATCCTGGTGCAGAAGGAGTGGATCAGCTTCGGCC |
| | ACAAGTTTGCCTCCAGGATCGGCCACGGCGATAAGAATCACACCGATGCC |
| | GACCGCAGCCCTATCTTCCTGCAGTTTATCGACTGCGTGTGGCAGATGTC |
| | CAAGCAGTTCCCAACCGCCTTCGAGTTTAACGAGCAGTTTCTGATCATCA |
| | TCCTGGACCACCTGTACTCCTGCAGGTTCGGCACATTCCTGTTTAATTGT |
| | GAGTCTGCCAGAGAGAGGCAGAAGGTGACCGAGCGCACAGTGTCTCTGTG |
| | GAGCCTGATCAACAGCAATAAGGAGAAGTTCAAGAACCCCTTCTACACAA |
| | AGGAGATCAATCGCGTGCTGTATCCTGTGGCATCCATGAGACACCTGGAG |
| | CTGTGGGTGAACTACTATATCCGCTGGAATCCCCGGATCAAGCAGCAGCA |
| | GCCCAACCCTGTGGAGCAGCGGTACATGGAGCTGCTGGCCCTGAGAGATG |
| | AGTATATCAAGAGGCTGGAGGAGCTGCAGCTGGCCAATAGCGCCAAGCTG |
| | TCCGACCCACCTACTTCACCAAGCAGCCCCTCACAGATGATGCCCCACGT |
| | CCAGACCCACTTTTAATTAAGATCTTTTTCCCTCTGCCAAAAATTATGGG |
| | GACATCATGAAGCCCCTTGAGCATCTGACTTCTGGCTAATAAAGGAAATT |
| | TATTTTCATTGCAATAGTGTGTTGGAATTTTTTGTGTCTCTCACTCGAGG |
| | CCTAGGAACCCCTAGTGATGGAGTTGGCCACTCCCTCTCTGCGCGCTCGC |
| | TCGCTCACTGAGGCCGGGCGACCAAAGGTCGCCCGACGCCCGGGCTTTGC |
| | CCGGGCGGCCTCAGTGAGCGAGCGAGCGCGCAGAGAGGGAGTGGCCAA |
| SEQ ID | TTGGCCACTCCCTCTCTGCGCGCTCGCTCGCTCACTGAGGCCGCCCGGGC |
| NO: | AAAGCCCGGGCGTCGGGCGACCTTTGGTCGCCCGGCCTCAGTGAGCGAGC |
| 187 | GAGCGCGCAGAGAGGGAGTGGCCAACTCCATCACTAGGGGTTCCTTGCGC |
| (E7) | ACAGGTACCGACATTGATTATTGACTAGTTATTAATAGTAATCAATTACG |
| | GGGTCATTAGTTCATAGCCCATATATGGAGTTCCGCGTTACATAACTTAC |
| | GGTAAATGGCCCGCCTGGCTGACCGCCCAACGACCCCCGCCCATTGACGT |
| | CAATAATGACGTATGTTCCCATAGTAACGCCAATAGGGACTTTCCATTGA |
| | CGTCAATGGGTGGAGTATTTACGGTAAACTGCCCACTTGGCAGTACATCA |
| | AGTGTATCATATGCCAAGTACGCCCCCTATTGACGTCAATGACGGTAAAT |
| | GGCCCGCCTGGCATTATGCCCAGTACATGACCTTATGGGACTTTCCTACT |
| | TGGCAGTACATCTACGTATTAGTCATCGCTATTACCATGGTCGAGGTGAG |
| | CCCCACGTTCTGCTTCACTCTCCCCATCTCCCCCCCCTCCCCACCCCCAA |
| | TTTTGTATTTATTTATTTTTTAATTATTTTGTGCAGCGATGGGGGGGGGG |
| | GGGGGGGGGGGGGGCGCGCCAGGGGGGCGGGGCGGGGCGAGGGGCGGGGG |
| | GGGGCGAGGCGGAGAGGTGCGGCGGCAGCCAATCAGAGCGGCGCGCTCCG |
| | AAAGTTTCCTTTTATGGCGAGGCGGCGGCGGCGGCGGCCCTATAAAAAGC |
| | GAAGCGCGCGGCGGGCGGATATCTCAGATCGCCTGGAGACGCCATCCACG |
| | CTGTTTTGACCTCCATAGAAGACACCGGGACCGATCCAGCCTCCGCGGAT |
| | TCGAATCCCGGCCGGGAACGGTGCATTGGAACGCGGATTCCCCGTGCCAA |
| | GAGTGACGTAAGTACCGCCTATAGAGTCTATAGGCCCACAAAAAATGCTT |
| | TCTTCTTTTAATATACTTTTTTGTTTATCTTATTTCTAATACTTTCCCTA |
| | ATCTCTTTCTTTCAGGGCAATAATGATACAATGTATCATGCCTCTTTGCA |
| | CCATTCTAAAGAATAACAGTGATAATTTCTGGGTTAAGGCAATAGCAATA |
| | TTTCTGCATATAAATATTTCTGCATATAAATTGTAACTGATGTAAGAGGT |
| | TTCATATTGCTAATAGCAGCTACAATCCAGCTACCATTCTGCTTTTATTT |
| | TATGGTTGGGATAAGGCTGGATTATTCTGAGTCCAAGCTAGGCCCTTTTG |
| | CTAATCATGTTCATACCTCTTATCTTCCTCCCACAGCTCCTGGGCAACGT |
| | GCTGGTCTGTGTGCTGGCCCATCACTTTGGCAAAGAATTTGAGCGGCCGC |
| | CACCATGGCTAGCGCCAGCACAAGCAAGTACAACAGCCACAGCCTGGAAA |
| | ACGAGAGCATCAAGCGGACCAGCAGAGATGGCGTGAACAGGGATCTGACA |
| | GAGGCCGTGCCTAGACTGCCTGGCGAGACACTGATCACCGACAAAGAAGT |
| | GATCTACATCTGCCCCTTCAACGGCCCCATCAAGGGCCGCGTGTACATCA |
| | CCAACTACCGGCTGTACCTGCGGTCCCTGGAAACCGATAGCAGCCTGATC |
| | CTGGATGTGCCCCTGGGCGTGATCAGCCGGATTGAGAAAATGGGCGGAGC |
| | CACCTCCAGAGGCGAGAATAGCTACGGCCTGGACATCACATGCAAGGACA |
| | TGCGGAACCTGAGATTCGCCCTGAAGCAAGAGGGCCACAGCAGACGGGAC |
| | ATGTTCGAGATCCTGACCAGATACGCCTTTCCTCTGGCTCACTCCCTGCC |
| | TCTGTTCGCCTTCCTGAACGAAGAGAAGTTCAACGTGGACGGCTGGACCG |
| | TGTACAACCCCGTGGAAGAGTATCGGAGACAGGGCCTGCCTAATCACCAC |
| | TGGQGGATCACCTTCATCAACAAGTGCTACGAGCTGTGCGACACATACCC |
| | CGCACTGCTGGTGGTGCCTTACAGAGCCTCCGACGACGACCTGAGAAGAG |
| | TGGCCACCTTTCGGAGCCGGAACAGAATCCCTGTGCTGAGCTGGATTCAC |
| | CCCGAGAACAAGACCGTGATCGTGCGGTGTTCTCAGCCCCTCGTGGGCAT |
| | GTCTGGCAAGAGAAACAAGGACGACGAGAAGTACCTGGACGTGATCCGCG |
| | AGACAAACAAGCAGATCAGCAAGCTGACCATCTACGACGCCAGACCTAGC |
| | GTGAACGCCGTGGCCAACAAAGCTACAGGCGGCGGATACGAGAGCGACGA |
| | CGCCTATCATAACGCCGAGCTGTTCTTTCTGGACATCCACAACATCCACG |
| | TGATGCGGGAAAGCCTGAAGAAAGTGAAGGACATCGTGTACCCCAATGTG |
| | GAAGAGAGCCACTGGCTGAGCAGTCTGGAAAGCACACACTGGCTGGAACA |
| | CATCAAGCTGGTGCTGACAGGCGCCATCCAGGTGGCCGATAAGGTGTCCT |
| | CTGGCAAGTCTAGCGTGCTGGTGCACTGTAGCGACGGCTGGGATAGAACA |
| | GCCCAGCTGACATCTCTGGCCATGCTGATGCTGGACAGCTTCTACAGATC |
| | CATCGAGGGCTTTGAGATCCTGGTGCAGAAAGAGTGGATCAGCTTCGGCC |
| | ACAAGTTCGCCAGCAGAATCGGACACGGCGACAAGAACCACACCGACGCC |
| | GATAGAAGCCCCATCTTCCTGCAGTTCATCGACTGCGTGTGGCAGATGTC |
| | CAAGCAGTTCCCTACCGCCTTCGAGTTCAACGAGCAGTTCCTGATCATCA |
| | TCCTGGACCACCTGTACAGCTGCAGATTCGGCACCTTCCTGTTCAACTGC |
| | GAGAGCGCCAGAGAACGGCAGAAAGTGACCGAGCGGACCGTGTCTCTGTG |
| | GTCCCTGATCAACAGCAACAAAGAGAAATTCAAGAACCCCTTCTACACCA |
| | AAGAAATCAACCGGGTGCTGTACCCCGTGGCCAGCATGAGACATCTGGAA |
| | CTGTGGGTCAACTACTACATCCGGTGGAACCCCAGAATCAAGCAGCAGCA |
| | GCCCAATCCTGTGGAACAGCGGTACATGGAACTGCTGGCCCTGCGGGACG |
| | AGTACATCAAGAGACTGGAAGAACTGCAGCTGGCCAACAGCGCCAAGCTG |
| | AGCGATCCTCCTACAAGCCCTAGCAGCCCCAGCCAGATGATGCCTCACGT |
| | GCAGACCCACTTTTAATTAAGATCTTTTTCCCTCTGCCAAAAATTATGGG |
| | GACATCATGAAGCCCCTTGAGCATCTGACTTCTGGCTAATAAAGGAAATT |
| | TATTTTCATTGCAATAGTGTGTTGGAATTTTTTGTGTCTCTCACTCGAGG |
| | CCTAGGAACCCCTAGTGATGGAGTTGGCCACTCCCTCTCTGCGCGCTCGC |
| | TCGCTCACTGAGGCCGGGCGACCAAAGGTCGCCCGACGCCCGGGCTTTGC |
| | CCGGGCGGCCTCAGTGAGCGAGCGAGCGCGCAGAGAGGGAGTGGCCAA |
| SEQ ID | TTGGCCACTCCCTCTCTGCGCGCTCGCTCGCTCACTGAGGCCGCCCGGGC |
| NO: | AAAGCCCGGGCGTCGGGCGACCTTTGGTCGCCCGGCCTCAGTGAGCGAGC |
| 188 | GAGCGCGCAGAGAGGGAGTGGCCAACTCCATCACTAGGGGTTCCTTGCGC |
| (E8) | ACAGGTACCGACATTGATTATTGACTAGTTATTAATAGTAATCAATTACG |
| | GGGTCATTAGTTCATAGCCCATATATGGAGTTCCGCGTTACATAACTTAC |
| | GGTAAATGGCCCGCCTGGCTGACCGCCCAACGACCCCCGCCCATTGACGT |
| | CAATAATGACGTATGTTQCCATAGTAACGCCAATAGGGACTTTCCATTGA |
| | CGTCAATGGGTGGAGTATTTACGGTAAACTGCCCACTTGGCAGTACATCA |
| | AGTGTATCATATGCCAAGTACGCCCCCTATTGACGTCAATGACGGTAAAT |
| | GGCCCGCCTGGCATTATGCCCAGTACATGACCTTATGGGACTTTCCTACT |
| | TGGCAGTACATCTACGTATTAGTCATCGCTATTACCATGGTCGAGGTGAG |
| | CCCCACGTTCTGCTTCACTCTCCCCATCTCCCCCCCCTCCCCACCCCCAA |
| | TTTTGTATTTATTTATTTTTTAATTATTTTGTGCAGCGATGGGGGGGGGG |
| | GGGGGGGGGGGGGGCGCGCCAGGQGGGGCGGGGCGGGGCGAGGGGGGGGG |
| | GGGGGCGAGGCGGAGAGGTGCGGGGCAGCCAATCAGAGCGGCGCGCTCCG |
| | AAAGTTTCCTTTTATGGCGAGGCGGCGGCGGCGGCGGCCCTATAAAAAGC |
| | GAAGCGCGCGGCGGGCGGATATCTCAGATCGCCTGGAGACGCCATCCACG |
| | CTGTTTTGACCTCCATAGAAGACACCGGGACCGATCCAGCCTCCGCGGAT |
| | TCGAATCCCGGCCGGGAACGGTGCATTGGAACGCGGATTCCCCGTGCCAA |
| | GAGTGACGTAAGTACCGCCTATAGAGTCTATAGGCCCACAAAAAATGCTT |
| | TCTTCTTTTAATATACTTTTTTGTTTATCTTATTTCTAATACTTTCCCTA |
| | ATCTCTTTCTTTCAGGGCAATAATGATACAATGTATCATGCCTCTTTGCA |
| | CCATTCTAAAGAATAACAGTGATAATTTCTGGGTTAAGGCAATAGCAATA |
| | TTTCTGCATATAAATATTTCTGCATATAAATTGTAACTGATGTAAGAGGT |
| | TTCATATTGCTAATAGCAGCTACAATCCAGCTACCATTCTGCTTTTATTT |
| | TATGGTTGGGATAAGGCTGGATTATTCTGAGTCCAAGCTAGGCCCTTTTG |
| | CTAATCATGTTCATACCTCTTATCTTCCTCCCACAGCTCCTGGGCAACGT |
| | GCTGGTCTGTGTGCTGGCCCATCACTTTGGCAAAGAATTTGAGCGGCCGC |
| | CACCATGGCTTCTGCATCAACTTCTAAATATAATTCACACTCCTTGGAGA |
| | ATGAGTCTATTAAGAGGACGTCTCGAGATGGAGTCAATCGAGATCTCACT |
| | GAGGCTGTTCCTCGACTTCCAGGAGAAACACTAATCACTGACAAAGAAGT |
| | TATTTACATATGTCCTTTCAATGGCCCCATTAAGGGAAGAGTTTACATCA |
| | CAAATTATCGTCTTTATTTAAGAAGTTTGGAAACGGATTCTTCTCTAATA |
| | CTTGATGTTCCTCTGGGTGTGATCTCGAGAATTGAAAAAATGGGAGGCGC |
| | GACAAGTAGAGGAGAAAATTCCTATGGTCTAGATATTACTTGTAAAGACA |
| | TGAGAAACCTGAGGTTCGCTTTGAAACAGGAAGGCCACAGCAGAAGAGAT |
| | ATGTTTGAGATCCTCACGAGATACGCGTTTCCCCTGGCTCACAGTCTGCC |
| | ATTATTTGCATTTTTAAATGAAGAAAAGTTTAACGTGGATGGATGGACAG |
| | TTTACAATCCAGTGGAAGAATACAGGAGGCAGGGCTTGCCCAATCACCAT |
| | TGGAGAATAACTTTTATTAATAAGTGCTATGAGCTCTGTGACACTTACCC |
| | TGCTCTTTTGGTGGTTCCGTATCGTGCCTCAGATGATGACCTCCGGAGAG |
| | TTGCAACTTTTAGGTCCCGAAATCGAATTCCAGTGCTGTCATGGATTCAT |
| | CCAGAAAATAAGACGGTCATTGTGCGTTGCAGTCAGCCTCTTGTCGGTAT |
| | GAGTGGGAAACGAAATAAAGATGATGAGAAATATCTCGATGTTATCAGGG |
| | AGACTAATAAACAAATTTCTAAACTCACCATTTATGATGCAAGACCCAGC |
| | GTAAATGCAGTGGCCAACAAGGCAACAGGAGGAGGATATGAAAGTGATGA |
| | TGCATATCATAACGCCGAACTTTTCTTCTTAGACATTCATAATATTCATG |
| | TTATGCGGGAATCTTTAAAAAAAGTGAAGGACATTGTTTATCCTAATGTA |
| | GAAGAATCTCATTGGTTGTCCAGTTTGGAGTCTACTCATTGGTTAGAACA |
| | TATCAAGCTCGTTTTGACAGGAGCCATTCAAGTAGCAGACAAAGTTTCTT |
| | CAGGGAAGAGTTCAGTGCTTGTGCATTGCAGTGACGGATGGGACAGGACT |
| | GCTCAGCTGACATCCTTGGCCATGCTGATGTTGGATAGCTTCTATAGGAG |
| | CATTGAAGGGTTCGAAATACTGGTACAAAAAGAATGGATAAGTTTTGGAC |
| | ATAAATTTGCATCTCGAATAGGTCATGGTGATAAAAACCACACCGATGCT |
| | GACCGTTCTCCTATTTTTCTCCAGTTTATTGATTGTGTGTGGCAAATGTC |
| | AAAACAGTTCCCTACAGCTTTTGAATTCAATGAACAATTTTTGATTATAA |
| | TTTTGGATCATCTGTATAGTTGCCGATTTGGTACTTTCTTATTCAACTGT |
| | GAATCTGCTCGAGAAAGACAGAAGGTTACAGAAAGGACTGTTTCTTTATG |
| | GTCACTGATAAACAGTAATAAAGAAAAATTCAAAAACCCATTCTATACTA |
| | AAGAAATCAATAGAGTTTTATATCCAGTTGCAAGTATGCGTCACTTGGAA |
| | CTCTGGGTGAATTACTACATTAGATGGAACCCCAGGATCAAACAACAACA |
| | ACCAAATCCAGTGGAACAACGTTACATGGAACTCTTAGCCTTACGAGATG |
| | AATACATAAAACGGCTTGAGGAACTGCAACTAGCAAACTCTGCAAAACTT |
| | TCTGATCCCCCAACTTCACCTTCCAGTCCTTCTCAAATGATGCCACATGT |
| | GCAAACTCACTTTTAATTAAGATCTTTTTCCCTCTGCCAAAAATTATGGG |
| | GACATCATGAAGCCCCTTGAGCATCTGACTTCTGGCTAATAAAGGAAATT |
| | TATTTTCATTGCAATAGTGTGTTGGAATTTTTTGTGTCTCTCACTCGAGG |
| | CCTAGGAACCCCTAGTGATGGAGTTGGCCACTCCCTCTCTGCGCGCTCGC |
| | TCGCTCACTGAGGCCGGGCGACCAAAGGTCGCCCGACGCCCGGGCTTTGC |
| | CCGGGCGGCCTCAGTGAGCGAGCGAGCGCGCAGAGAGGGAGTGGCCAA |
| SEQ ID | TTGGCCACTCCCTCTCTGCGCGCTCGCTCGCTCACTGAGGCCGCCCGGGC |
| NO: | AAAGCCCGGGCGTCGGGCGACCTTTGGTCGCCCGGCCTCAGTGAGCGAGC |
| 189 | GAGCGCGCAGAGAGGGAGTGGCCAACTCCATCACTAGGGGTTCCTTGCGC |
| (E9) | ACAGGTACCGACATTGATTATTGACTAGTTATTAATAGTAATCAATTACG |
| | GGGTCATTAGTTCATAGCCCATATATGGAGTTCCGCGTTACATAACTTAC |
| | GGTAAATGGCCCGCCTGGCTGACCGCCCAACGACCCCCGCCCATTGACGT |
| | CAATAATGACGTATGTTCCCATAGTAACGCCAATAGGGACTTTCCATTGA |
| | CGTCAATGGGTGGAGTATTTACGGTAAACTGCCCACTTGGCAGTACATCA |
| | AGTGTATCATATGCCAAGTACGCCCCCTATTGACGTCAATGACGGTAAAT |
| | GGCCCGCCTGGCATTATGCCCAGTACATGACCTTATGGGACTTTCCTACT |
| | TGGCAGTACATCTACGTATTAGTCATCGCTATTACCATGGTCGAGGTGAG |
| | CCCCACGTTCTGCTTCACTCTCCCCATCTCCCCCCCCTCCCCACCCCCAA |
| | TTTTGTATTTATTTATTTTTTAATTATTTTGTGCAGCGATGGGGGGGGGG |
| | GGGGGGGGGGGGGGCGCGCCAGGCGGGGCGGGGGGGGCGAGGGGCGGGGC |
| | GGGGCGAGGCGGAGAGGTGCGGCGGCAGCCAATCAGAGCGGCGCGCTCCG |
| | AAAGTTTCCTTTTATGGCGAGGCGGCGGCGGCGGCGGCCCTATAAAAAGC |
| | GAAGCGCGCGGGGGGGGATATCTCAGATCGCCTGGAGACGCCATCCACGC |
| | TGTTTTGACCTCCATAGAAGACACCGGGACCGATCCAGCCTCCGCGGATT |
| | CGAATCCCGGCCGGGAACGGTGCATTGGAACGCGGATTCCCCGTGCCAAG |
| | AGTGACGTAAGTACCGCCTATAGAGTCTATAGGCCCACAAAAAATGCTTT |
| | CTTCTTTTAATATACTTTTTTGTTTATCTTATTTCTAATACTTTCCCTAA |
| | TCTCTTTCTTTCAGGGCAATAATGATACAATGTATCATGCCTCTTTGCAC |
| | CATTCTAAAGAATAACAGTGATAATTTCTGGGTTAAGGCAATAGCAATAT |
| | TTCTGCATATAAATATTTCTGCATATAAATTGTAACTGATGTAAGAGGTT |
| | TCATATTGCTAATAGCAGCTACAATCCAGCTACCATTCTGCTTTTATTTT |
| | ATGGTTGGGATAAGGCTGGATTATTCTGAGTCCAAGCTAGGCCCTTTTGC |
| | TAATCATGTTCATACCTCTTATCTTCCTQCCACAGCTCCTGGGCAACGTG |
| | CTGGTCTGTGTGCTGGCCCATCACTTTGGCAAAGAATTTGAGCGGCCGCC |
| | ACCATGGCTTCTGCATCAACTTCTAAATATAATTCACACTCCTTGGAGAA |
| | TGAGTCTATTAAGAGGACGTCTCGAGATGGAGTCAATCGAGATCTCACTG |
| | AGGCTGTTCCTCGACTTCCAGGAGAAACACTAATCACTGACAAAGAAGTT |
| | ATTTACATATGTCCTTTCAATGGCCCCATTAAGGGAAGAGTTTACATCAC |
| | AAATTATCGTCTTTATTTAAGAAGTTTGGAAACGGATTCTTCTCTAATAC |
| | TTGATGTTQCTCTGGGTGTGATCTCGAGAATTGAAAAAATGGGAGGCGCG |
| | ACAAGTAGAGGAGAAAATTCCTATGGTCTAGATATTACTTGTAAAGACAT |
| | GAGAAACCTGAGGTTCGCTTTGAAACAGGAAGGCCACAGCAGAAGAGATA |
| | TGTTTGAGATCCTCACGAGATACGCGTTTCCCCTGGCTCACAGTCTGCCA |
| | TTATTTGCATTTTTAAATGAAGAAAAGTTTAACGTGGATGGATGGACAGT |
| | TTACAATCCAGTGGAAGAATACAGGAGGCAGGGCTTGCCCAATCACCATT |
| | GGAGAATAACTTTTATTAATAAGTGCTATGAGCTCTGTGACACTTACCCT |
| | GCTCTTTTGGTGGTTCCGTATCGTGCCTCAGATGATGACCTCCGGAGAGT |
| | TGCAACTTTTAGGTCCCGAAATCGAATTCCAGTGCTGTCATGGATTCATC |
| | CAGAAAATAAGACGGTCATTGTGCGTTGCAGTCAGCCTCTTGTCGGTATG |
| | AGTGGGAAACGAAATAAAGATGATGAGAAATATCTCGATGTTATCAGGGA |
| | GACTAATAAACAAATTTCTAAACTCACCATTTATGATGCAAGACCCAGCG |
| | TAAATGCAGTGGCCAACAAGGCAACAGGAGGAGGATATGAAAGTGATGAT |
| | GCATATCATAACGCCGAACTTTTCTTCTTAGACATTCATAATATTCATGT |
| | TATGCGGGAATCTTTAAAAAAAGTGAAGGACATTGTTTATCCTAATGTAG |
| | AAGAATCTCATTGGTTGTCCAGTTTGGAGTCTACTCATTGGTTAGAACAT |
| | ATCAAGCTQGTTTTGACAGGAGCCATTCAAGTAGCAGACAAAGTTTCTTC |
| | AGGGAAGAGTTCAGTGCTTGTGCATTGCAGTGACGGATGGGACAGGACTG |
| | CTCAGCTGACATCCTTGGCCATGCTGATGTTGGATAGCTTCTATAGGAGC |
| | ATTGAAGGGTTCGAAATACTGGTACAAAAAGAATGGATAAGTTTTGGACA |
| | TAAATTTGCATCTCGAATAGGTCATGGTGATAAAAACCACACCGATGCTG |
| | ACCGTTCTCCTATTTTTCTCCAGTTTATTGATTGTGTGTGGCAAATGTCA |
| | AAACAGTTCCCTACAGCTTTTGAATTCAATGAACAATTTTTGATTATAAT |
| | TTTGGATCATCTGTATAGTTGCCGATTTGGTACTTTCTTATTCAACTGTG |
| | AATCTGCTCGAGAAAGACAGAAGGTTACAGAAAGGACTGTTTCTTTATGG |
| | TCACTGATAAACAGTAATAAAGAAAAATTCAAAAACCCCTTCTATACTAA |
| | AGAAATCAATCGAGTTTTATATCCAGTTGCCAGTATGCGTCACTTGGAAC |
| | TCTGGGTGAATTACTACATTAGATGGAACCCCAGGATCAAGCAACAACAG |
| | CCGAATCCAGTGGAGCAGCGTTACATGGAGCTCTTAGCCTTACGCGACGA |
| | ATACATAAAGCGGCTTGAGGAACTGCAGCTCGCCAACTCTGCCAAGCTTT |
| | CTGATCCCCCAACTTCACCTTCCAGTCCTTCGCAAATGATGCCCCATGTG |
| | CAAACTCACTTTTAATTAAGATCTTTTTCCCTCTGCCAAAAATTATGGGG |
| | ACATCATGAAGCCCCTTGAGCATCTGACTTCTGGCTAATAAAGGAAATTT |
| | ATTTTCATTGCAATAGTGTGTTGGAATTTTTTGTGTCTCTCACTCGAGGC |
| | CTAGGAACCCCTAGTGATGGAGTTGGCCACTCCCTCTCTGCGCGCTCGCT |
| | CGCTCACTGAGGCCGGGCGACCAAAGGTCGCCCGACGCCCGGGCTTTGCC |
| | CGGGCGGCCTCAGTGAGCGAGCGAGCGCGCAGAGAGGGAGTGGCCAA |
| SEQ ID | TTGTCGACTCGGTTCGCATATTAAGGTGACGCGTGTGGCCTCGAACACCG |
| NO: | AGCGACCCTGCAGCGACCCGCTTAA |
| 190 | |
| (E10A) | |
| SEQ ID | CCACTCCCTCTCTGCGCGCTCGCTCGCTCACTGAGGCCGGGCGACCAAAG |
| NO: | GTCGCCCGACGCCCGGGCTTTGCCCGGGCGGCCTCAGTGAGCGAGCGAGC |
| 191 | GCGCAGAGAGGGAGTGGCCAA |
| (E10B) | |
| SEQ ID | TTTTTTGGTACCTTCGCATATTAAGGTGACGCGTGTGGCCTCGAACACCG |
| NO: | AGCGACCCTGCAGCGACCCGCTTAAGCGGCCGCCACCATGGCATCAGCAA |
| 192 | GCACAAGCAAATACAACTCACATAGCCTGGAAAACG |
| (EIIA) | |
| SEQ ID | AGTGAGCGAGCGAGCGCGCAGAGAGGGAGTGGCCAACTCCATCACTAGGG |
| NO: | GTTCCTTGCGCACAGGTACCTTCGCATATTAAGGTGACGCGTGTGGCCTC |
| 193 | GAACACCGAGCGACCCTGCAGCGACCCGCTTAAGCGGCCGCCACCATGGC |
| (E11B) | TTCTGCATCAACTTCTAAATATAATTCACACTCCTTGGAGAATGAGTCTA |
| | TTAAGAGGACGTCTCGAGATGGAGTCAATCGAGATCTCACTGAGGCTGTT |
| | CCTCGACTTCCAGGAGAAACACTAATCACTGACAAAGAAGTTATTTACAT |
| | ATGTCCTTTCAATGGCCCCATTAAGGGAAGAGTTTACATCACAAATTATC |
| | GTCTTTATTTAAGAAGTTTGGAAACGGATTCTTCTCTAATACTTGATGTT |
| | CCTCTGGGTGTGATCTCGAGAATTGAAAAAATGGGAGGCGCGACAAGTAG |
| | AGGAGAAAATTCCTATGGTCTAGATATTACTTGTAAAGACATGAGAAACC |
| | TGAGGTTCGCTTTGAAACAGGAAGGCCACAGCAGAAGAGATATGTTTGAG |
| | ATCCTCACGAGATACGQGTTTCCCCTGGCTCACAGTCTGCCATTATTTGC |
| | ATTTTTAAATGAAGAAAAGTTTAACGTGGATGGATGGACAGTTTACAATC |
| | CAGTGGAAGAATACAGGAGGCAGGGCTTGCCCAATCACCATTGGAGAATA |
| | ACTTTTATTAATAAGTGCTATGAGCTCTGTGACACTTACCCTGCTCTTTT |
| | GGTGGTTCCGTATCGTGCCTCAGATGATGACCTCCGGAGAGTTGCAACTT |
| | TTAGGTCCCGAAATCGAATTCCAGTGCTGTCATGGATTCATCCAGAAAAT |
| | AAGACGGTCATTGTGCGTTGCAGTCAGCCTCTTGTCGGTATGAGTGGGAA |
| | ACGAAATAAAGATGATGAGAAATATCTCGATGTTATCAGGGAGACTAATA |
| | AACAAATTTCTAAACTCACCATTTATGATGCAAGACCCAGCGTAAATGCA |
| | GTGGCCAACAAGGCAACAGGAGGAGGATATGAAAGTGATGATGCATATCA |
| | TAACGCCGAACTTTTCTTCTTAGACATTCATAATATTCATGTTATGCGGG |
| | AATCTTTAAAAAAAGTGAAGGACATTGTTTATCCTAATGTAGAAGAATCT |
| | CATTGGTTGTCCAGTTTGGAGTCTACTCATTGGTTAGAACATATCAAGCT |
| | CGTTTTGACAGGAGCCATTCAAGTAGCAGACAAAGTTTCTTCAGGGAAGA |
| | GTTCAGTGCTTGTGCATTGCAGTGACGGATGGGACAGGACTGCTCAGCTG |
| | ACATCCTTGGCCATGCTGATGTTGGATAGCTTCTATAGGAGCATTGAAGG |
| | GTTCGAAATACTGGTACAAAAAGAATGGATAAGTTTTGGACATAAATTTG |
| | CATCTCGAATAGGTCATGGTGATAAAAACCACACCGATGCTGACCGTTCT |
| | CCTATTTTTCTCCAGTTTATTGATTGTGTGTGGCAAATGTCAAAACAGTT |
| | CCCTACAGCTTTTGAATTCAATGAACAATTTTTGATTATAATTTTGGATC |
| | ATCTGTATAGTTGCCGATTTGGTACTTTCTTATTCAACTGTGAATCTGCT |
| | CGAGAAAGACAGAAGGTTACAGAAAGGACTGTTTCTTTATGGTCACTGAT |
| | AAACAGTAATAAAGAAAAATTCAAAAACCCCTTCTATACTAAAGAAATCA |
| | ATCGAGTTTTATATCCAGTTGCCAGTATGCGTCACTTGGAACTCTGGGTG |
| | AATTACTACATTAGATGGAACCCCAGGATCAAGCAACAACAGCCGAATCC |
| | AGTGGAGCAGCGTTACATGGAGCTCTTAGCCTTACGCGACGAATACATAA |
| | AGCGGCTTGAGGAACTGCAGCTCGCCAACTCTGCCAAGCTTTCTGATCCC |
| | CCAACTTCACCTTCCAGTCCTTCGCAAATGATGCCCCATGTGCAAACTCA |
| | CTTTTAATTAAGATCTTTTTCCCTCTGCCAAAAATTATGGGGACATCATG |
| | AAGCCCCTTGAGCATCTGACTTCTGGCTAATAAAGGAAATTTATTTTCAT |
| | TGCAATAGTGTGTTGGAATTTTTTGTGTCTCTCACTCGAGGCCTAGGAAC |
| | CCCTAGTGATGGAGTTGGCCACTCCCTCTCTGCGCGCTCGCTCGCTCACT |
| | GAGGCCGGGCGACCAAAGGTCGCCCGACGCCCGGGCTTTGCCCGGGCGGC |
| | CTCAGTGAGCGAGCGAGCGCGCAGAGAGGGAGTGGCCAA |
| SEQ ID | TTTTTGTCGACTTCGCATATTAAGGTGACGCGTGTGGCCTCGAACACCGA |
| NO: | GCGACCCTGCAGCGACCCGCTTAAGCGGCCGCCACCATGGCTTCTGCATC |
| 194 | AACTTCTAAATATAATTCACACTCCTTGGAGAATGAGTCTATTAAGAGGA |
| (E12) | CGTCTCGAGATGGAGTCAATCGAGATCTCACTGAGGCTGTTCCTCGACTT |
| | CCAGGAGAAACACTAATCACTGACAAAGAAGTTATTTACATATGTCCTTT |
| | CAATGGCCCCATTAAGGGAAGAGTTTACATCACAAATTATCGTCTTTATT |
| | TAAGAAGTTTGGAAACGGATTCTTCTCTAATACTTGATGTTCCTCTGGGT |
| | GTGATCTCGAGAATTGAAAAAATGGGAGGCGCGACAAGTAGAGGAGAAAA |
| | TTCCTATGGTCTAGATATTACTTGTAAAGACATGAGAAACCTGAGGTTCG |
| | CTTTGAAACAGGAAGGCCACAGCAGAAGAGATATGTTTGAGATCCTCACG |
| | AGATACGCGTTTCCCCTGGCTCACAGTCTGCCATTATTTGCATTTTTAAA |
| | TGAAGAAAAGTTTAACGTGGATGGATGGACAGTTTACAATCCAGTGGAAG |
| | AATACAGGAGGCAGGGCTTGCCCAATCACCATTGGAGAATAACTTTTATT |
| | AATAAGTGCTATGAGCTCTGTGACACTTACCCTGCTCTTTTGGTGGTTCC |
| | GTATCGTGCCTCAGATGATGACCTCCGGAGAGTTGCAACTTTTAGGTCCC |
| | GAAATCGAATTCCAGTGCTGTCATGGATTCATCCAGAAAATAAGACGGTC |
| | ATTGTGCGTTGCAGTCAGCCTCTTGTQGGTATGAGTGGGAAACGAAATAA |
| | AGATGATGAGAAATATCTCGATGTTATCAGGGAGACTAATAAACAAATTT |
| | CTAAACTCACCATTTATGATGCAAGACCCAGCGTAAATGCAGTGGCCAAC |
| | AAGGCAACAGGAGGAGGATATGAAAGTGATGATGCATATCATAACGCCGA |
| | ACTTTTCTTCTTAGACATTCATAATATTCATGTTATGCGGGAATCTTTAA |
| | AAAAAGTGAAGGACATTGTTTATCCTAATGTAGAAGAATCTCATTGGTTG |
| | TCCAGTTTGGAGTCTACTCATTGGTTAGAACATATCAAGCTCGTTTTGAC |
| | AGGAGCCATTCAAGTAGCAGACAAAGTTTCTTCAGGGAAGAGTTCAGTGC |
| | TTGTGCATTGCAGTGACGGATGGGACAGGACTGCTCAGCTGACATCCTTG |
| | GCCATGCTGATGTTGGATAGCTTCTATAGGAGCATTGAAGGGTTCGAAAT |
| | ACTGGTACAAAAAGAATGGATAAGTTTTGGACATAAATTTGCATCTCGAA |
| | TAGGTCATGGTGATAAAAACCACACCGATGCTGACCGTTCTCCTATTTTT |
| | CTCCAGTTTATTGATTGTGTGTGGCAAATGTCAAAACAGTTCCCTACAGC |
| | TTTTGAATTCAATGAACAATTITTGATTATAATTTTGGATCATCTGTATA |
| | GTTGCCGATTTGGTACTTTCTTATTCAACTGTGAATCTGCTCGAGAAAGA |
| | CAGAAGGTTACAGAAAGGACTGTTTCTTTATGGTCACTGATAAACAGTAA |
| | TAAAGAAAAATTCAAAAACCCCTTCTATACTAAAGAAATCAATCGAGTTT |
| | TATATCCAGTTGCCAGTATGCGTCACTTGGAACTCTGGGTGAATTACTAC |
| | ATTAGATGGAACCCCAGGATCAAGCAACAACAGCCGAATCCAGTGGAGCA |
| | GCGTTACATGGAGCTCTTAGCCTTACGCGACGAATACATAAAGCGGCTTG |
| | AGGAACTGCAGCTCGCCAACTCTGCCAAGCTTTCTGATCCCCCAACTTCA |
| | CCTTCCAGTCCTTCGCAAATGATGCCCCATGTGCAAACTCACTTTTAATT |
| | AAGATCTTTTTCCCTCTGCCAAAAATTATGGGGACATCATGAAGCCCCTT |
| | GAGCATCTGACTTCTGGCTAATAAAGGAAATTTATTTTCATTGCAATAGT |
| | GTGTTGGAATTTTTTGTGTCTCTCACTCCCTAGGAAAAAA |
| SEQ ID | TTTTTGTCGACTTCGCATATTAAGGTGACGCGTGTGGCCTCGAACACCGA |
| NO: | GQGACCCTGCAGCGACCCGCTTAAGCGGCCGCCACCATGGCTTCTGCATC |
| 195 | AACTTCTAAATATAATTCACACTCCTTGGAGAATGAGTCTATTAAGAGGA |
| (E13A) | CGTCTCGAGATGGAGTCAATCGAGATCTCACTGAGGCTGTTCCTCGACTT |
| | CCAGGAGAAACACTAATCACTGACAAAGAAGTTATTTACATATGTCCTTT |
| | CAATGGCCCCATTAAGGGAAGAGTTTACATCACAAATTATCGTCTTTATT |
| | TAAGAAGTTTGGAAACGGATTCTTCTCTAATACTTGATGTTCCTCTGGGT |
| | GTGATCTCGAGAATTGAAAAAATGGGAGGCGCGACAAGTAGAGGAGAAAA |
| | TTCCTATGGTCTAGATATTACTTGTAAAGACATGAGAAACCTGAGGTTCG |
| | CTTTGAAACAGGAAGGCCACAGCAGAAGAGATATGTTTGAGATCCTCACG |
| | AGATACGCGTTTCCCCTGGCTCACAGTCTGCCATTATTTGCATTTTTAAA |
| | TGAAGAAAAGTTTAACGTGGATGGATGGACAGTTTACAATCCAGTGGAAG |
| | AATACAGGAGGCAGGGCTTGCCCAATCACCATTGGAGAATAACTTTTATT |
| | AATAAGTGCTATGAGCTCTGTGACACTTACCCTGCTCTTTTGGTGGTTCC |
| | GTATCGTGCCTCAGATGATGACCTCCGGAGAGTTGCAACTTTTAGGTCCC |
| | GAAATCGAATTCCAGTGCTGTCATGGATTCATCCAGAAAATAAGACGGTC |
| | ATTGTGCGTTGCAGTCAGCCTCTTGTCGGTATGAGTGGGAAACGAAATAA |
| | AGATGATGAGAAATATCTCGATGTTATCAGGGAGACTAATAAACAAATTT |
| | CTAAACTCACCATTTATGATGCAAGACCCAGCGTAAATGCAGTGGCCAAC |
| | AAGGCAACAGGAGGAGGATATGAAAGTGATGATGCATATCATAACGCCGA |
| | ACTTTTCTTCTTAGACATTCATAATATTCATGTTATGCGGGAATCTTTAA |
| | AAAAAGTGAAGGACATTGTTTATCCTAATGTAGAAGAATCTCATTGGTTG |
| | TCCAGTTTGGAGTCTACTCATTGGTTAGAACATATCAAGCTCGTTTTGAC |
| | AGGAGCCATTCAAGTAGCAGACAAAGTTTCTTCAGGGAAGAGTTCAGTGC |
| | TTGTGCATTGCAGTGACGGATGGGACAGGACTGCTCAGCTGACATCCTTG |
| | GCCATGCTGATGTTGGATAGCTTCTATAGGAGCATTGAAGGGTTCGAAAT |
| | ACTGGTACAAAAAGAATGGATAAGTTTTGGACATAAATTTGCATCTCGAA |
| | TAGGTCATGGTGATAAAAACCACACCGATGCTGACCGTTCTCCTATTTTT |
| | CTCCAGTTTATTGATTGTGTGTGGCAAATGTCAAAACAGTTCCCTACAGC |
| | TTTTGAATTCAATGAACAATTTTTGATTATAATTTTGGATCATCTGTATA |
| | GTTGCCGATTTGGTACTTTCTTATTCAACTGTGAATCTGCTCGAGAAAGA |
| | CAGAAGGTTACAGAAAGGACTGITTCTTTATGGTCACTGATAAACAGTAA |
| | TAAAGAAAAATTCAAAAACCCCTTCTATACTAAAGAAATCAATCGAGTTT |
| | TATATCCAGTTGCCAGTATGCGTCACTTGGAACTCTGGGTGAATTACTAC |
| | ATTAGATGGAACCCCAGGATCAAGCAACAACAGCCGAATCCAGTGGAGCA |
| | GCGTTACATGGAGCTCTTAGCCTTACGCGACGAATACATAAAGCGGCTTG |
| | AGGAACTGCAGCTCGCCAACTCTGCCAAGCTTTCTGATCCCCCAACTTCA |
| | CCTTCCAGTCCTTCGCAAATGATGCCCCATGTGCAAACTCACTTTTAATT |
| | AAGATCTTTTTCCCTCTGCCAAAAATTATGGGGACATCATGAAGCCCCTT |
| | GAGCATCTGACTTCTGGCTAATAAAGGAAATTTATTTTCATTGCAATAGT |
| | GTGTTGGAATTTTTTGTGTCTCTCACTCCCTAGGAAAAAA |
| SEQ ID | TTTTTGTCGACCCTCTATAAATACCCGCTCTGGGTTGGCAGCTGTTGCTG |
| NO: | CGGTGTGTGTGGGCGTGGGTGGTGTGAGTAGGGGGATGAATCAGGGAGGG |
| 196 | GGCGGGGGCAGGGGGCAGGAGCCACACAAACCTGCCCTGGCGAAGACCCC |
| (E13B) | CGCTGGCTGACTCAGGGATCTTGCAGCTGTCAGGGGGGAGGGATACAAAT |
| | AGTGCCGACGGCTGGGGGCCCTGTCTCCCCTCGCCGCGGCCGCCACCATG |
| | GCTTCTGCATCAACTTCTAAATATAATTCACACTCCTTGGAGAATGAGTC |
| | TATTAAGAGGACGTCTCGAGATGGAGTCAATCGAGATCTCACTGAGGCTG |
| | TTCCTCGACTTCCAGGAGAAACACTAATCACTGACAAAGAAGTTATTTAC |
| | ATATGTCCTTICAATGGCCCCATTAAGGGAAGAGTTTACATCACAAATTA |
| | TCGTCTTTATTTAAGAAGTTTGGAAACGGATTCTTCTCTAATACTTGATG |
| | TTCCTCTGGGTGTGATCTCGAGAATTGAAAAAATGGGAGGCGCGACAAGT |
| | AGAGGAGAAAATTCCTATGGTCTAGATATTACTTGTAAAGACATGAGAAA |
| | CCTGAGGTTCGCTTTGAAACAGGAAGGCCACAGCAGAAGAGATATGTTTG |
| | AGATCCTCACGAGATACGCGTTTCCCCTGGCTCACAGTCTGCCATTATTT |
| | GCATTTTTAAATGAAGAAAAGTTTAACGTGGATGGATGGACAGTTTACAA |
| | TCCAGTGGAAGAATACAGGAGGCAGGGCTTGCCCAATCACCATTGGAGAA |
| | TAACTTTTATTAATAAGTGCTATGAGCTCTGTGACACTTACCCTGCTCTT |
| | TTGGTGGTTCCGTATCGTGCCTCAGATGATGACCTCCGGAGAGTTGCAAC |
| | TTTTAGGTCCCGAAATCGAATTCCAGTGCTGTCATGGATTCATCCAGAAA |
| | ATAAGACGGTCATTGTGCGTTGCAGTCAGCCTCTTGTCGGTATGAGTGGG |
| | AAACGAAATAAAGATGATGAGAAATATCTCGATGTTATCAGGGAGACTAA |
| | TAAACAAATTTCTAAACTCACCATTTATGATGCAAGACCCAGCGTAAATG |
| | CAGTGGCCAACAAGGCAACAGGAGGAGGATATGAAAGTGATGATGCATAT |
| | CATAACGCCGAACTTTTCTTCTTAGACATTCATAATATTCATGTTATGCG |
| | GGAATCTTTAAAAAAAGTGAAGGACATTGTTTATCCTAATGTAGAAGAAT |
| | CTCATTGGTTGTCCAGTTTGGAGTCTACTCATTGGTTAGAACATATCAAG |
| | CTCGTTTTGACAGGAGCCATTCAAGTAGCAGACAAAGTTTCTTCAGGGAA |
| | GAGTTCAGTGCTTGTGCATTGCAGTGACGGATGGGACAGGACTGCTCAGC |
| | TGACATCCTTGGCCATGCTGATGTTGGATAGCTTCTATAGGAGCATTGAA |
| | GGGTTCGAAATACTGGTACAAAAAGAATGGATAAGTTTTGGACATAAATT |
| | TGCATCTCGAATAGGTCATGGTGATAAAAACCACACCGATGCTGACCGTT |
| | CTCCTATTTTTCTCCAGTTTATTGATTGTGTGTGGCAAATGTCAAAACAG |
| | TTCCCTACAGCTTTTGAATTCAATGAACAATTTTTGATTATAATTTTGGA |
| | TCATCTGTATAGTTGCCGATTTGGTACTTTCTTATTCAACTGTGAATCTG |
| | CTCGAGAAAGACAGAAGGTTACAGAAAGGACTGTTTCTTTATGGTCACTG |
| | ATAAACAGTAATAAAGAAAAATTCAAAAACCCCTTCTATACTAAAGAAAT |
| | CAATCGAGTTTTATATCCAGTTGCCAGTATGCGTCACTTGGAACTCTGGG |
| | TGAATTACTACATTAGATGGAACCCCAGGATCAAGCAACAACAGCCGAAT |
| | CCAGTGGAGCAGCGTTACATGGAGCTCTTAGCCTTACGCGACGAATACAT |
| | AAAGCGGCTTGAGGAACTGCAGCTCGCCAACTCTGCCAAGCTTTCTGATC |
| | CCCCAACTTCACCTTCCAGTCCTTCGCAAATGATGCCCCATGTGCAAACT |
| | CACTTTTAATTAAGATCTTTTTCCCTCTGCCAAAAATTATGGGGACATCA |
| | TGAAGCCCCTTGAGCATCTGACTTCTGGCTAATAAAGGAAATTTATTTTC |
| | ATTGCAATAGTGTGTTGGAATTTTTTGTGTCTCTCACTCCCTAGGAAAAA |
| | A |
| SEQ ID | AGTGAGCGAGCGAGCGCGCAGAGAGGGAGTGGCCAACTCCATCACTAGGG |
| NO: | GTTCCTTGCGCACAGGTACCCCTCTATAAATACCCGCTCTGGGTTGGCAG |
| 197 | CTGTTGCTGCGGTGTGTGTGGGCGTGGGTGGTGTGAGTAGGGGGATGAAT |
| (E13C) | CAGGGAGGGGGCGGGGGCAGGGGGCAGGAGCCACACAAACCTGCCCTGGC |
| | GAAGACCCCCGCTGGCTGACTCAGGGATCTTGCAGCTGTCAGGGGGGAGG |
| | GATACAAATAGTGCCGACGGCTGGGGGCCCTGTCTCCCCTCGCCGCGGCC |
| | GCCACCATGGCTTCTGCATCAACTTCTAAATATAATTCACACTCCTTGGA |
| | GAATGAGTCTATTAAGAGGACGTCTCGAGATGGAGTCAATCGAGATCTCA |
| | CTGAGGCTGTTCCTCGACTTCCAGGAGAAACACTAATCACTGACAAAGAA |
| | GTTATTTACATATGTCCTTTCAATGGCCCCATTAAGGGAAGAGTTTACAT |
| | CACAAATTATCGTCTTTATTTAAGAAGTTTGGAAACGGATTCTTCTCTAA |
| | TACTTGATGTTCCTCTGGGTGTGATCTCGAGAATTGAAAAAATGGGAGGC |
| | GCGACAAGTAGAGGAGAAAATTCCTATGGTCTAGATATTACTTGTAAAGA |
| | CATGAGAAACCTGAGGTTCGCTTTGAAACAGGAAGGCCACAGCAGAAGAG |
| | ATATGTTTGAGATCCTCACGAGATACGCGTTTCCCCTGGCTCACAGTCTG |
| | CCATTATTTGCATTTTTAAATGAAGAAAAGTTTAACGTGGATGGATGGAC |
| | AGTTTACAATCCAGTGGAAGAATACAGGAGGCAGGGCTTGCCCAATCACC |
| | ATTGGAGAATAACTTTTATTAATAAGTGCTATGAGCTCTGTGACACTTAC |
| | CCTGCTCTTTTGGTGGTTCCGTATCGTGCCTCAGATGATGACCTCCGGAG |
| | AGTTGCAACTTTTAGGTCCCGAAATCGAATTCCAGTGCTGTCATGGATTC |
| | ATCCAGAAAATAAGACGGTCATTGTGCGTTGCAGTCAGCCTCTTGTCGGT |
| | ATGAGTGGGAAACGAAATAAAGATGATGAGAAATATCTCGATGTTATCAG |
| | GGAGACTAATAAACAAATTTCTAAACTCACCATTTATGATGCAAGACCCA |
| | GCGTAAATGCAGTGGCCAACAAGGCAACAGGAGGAGGATATGAAAGTGAT |
| | GATGCATATCATAACGCCGAACTTTTCTTCTTAGACATTCATAATATTCA |
| | TGTTATGCGGGAATCTTTAAAAAAAGTGAAGGACATTGTTTATCCTAATG |
| | TAGAAGAATCTCATTGGTTGTCCAGTTTGGAGTCTACTCATTGGTTAGAA |
| | CATATCAAGCTQGTTTTGACAGGAGCCATTCAAGTAGCAGACAAAGTTTC |
| | TTCAGGGAAGAGTTCAGTGCTTGTGCATTGCAGTGACGGATGGGACAGGA |
| | CTGCTCAGCTGACATCCTTGGCCATGCTGATGTTGGATAGCTTCTATAGG |
| | AGCATTGAAGGGTTCGAAATACTGGTACAAAAAGAATGGATAAGTTTTGG |
| | ACATAAATTTGCATCTCGAATAGGTCATGGTGATAAAAACCACACCGATG |
| | CTGACCGTTCTCCTATTTTTCTCCAGTTTATTGATTGTGTGTGGCAAATG |
| | TCAAAACAGTTCCCTACAGCTTTTGAATTCAATGAACAATTTTTGATTAT |
| | AATTTTGGATCATCTGTATAGTTGCCGATTTGGTACTTTCTTATTCAACT |
| | GTGAATCTGCTCGAGAAAGACAGAAGGTTACAGAAAGGACTGITTCTTTA |
| | TGGTCACTGATAAACAGTAATAAAGAAAAATTCAAAAACCCCTTCTATAC |
| | TAAAGAAATCAATCGAGTTTTATATCCAGTTGCCAGTATGCGTCACTTGG |
| | AACTCTGGGTGAATTACTACATTAGATGGAACCCCAGGATCAAGCAACAA |
| | CAGCCGAATCCAGTGGAGCAGCGTTACATGGAGCTCTTAGCCTTACGCGA |
| | CGAATACATAAAGCGGCTTGAGGAACTGCAGCTCGCCAACTCTGCCAAGC |
| | TTTCTGATCCCCCAACTTCACCTTCCAGTCCTTCGCAAATGATGCCCCAT |
| | GTGCAAACTCACTTTTAATTAAGATCTTTTTCCCTCTGCCAAAAATTATG |
| | GGGACATCATGAAGCCCCTTGAGCATCTGACTTCTGGCTAATAAAGGAAA |
| | TTTATTTTCATTGCAATAGTGTGTTGGAATTTTTTGTGTCTCTCACTCGA |
| | GGCCTAGGAACCCCTAGTGATGGAGTTGGCCACTCCCTCTCTGCGCGCTC |
| | GCTCGCTCACTGAGGCCGGGCGACCAAAGGTCGCCCGACGCCCGGGCTTT |
| | GCCCGGGCGGCCTCAGTGAGCGAGCGAGCGCGCAGAGAGGGAGTGGCCAA |
| SEQ ID | TTTTTGTCGACCCTCTATAAATACCCGCTCTGGGTTGGCAGCTGTTGCTG |
| NO: | CGGTGTGTGTGGGCGTGGGTGGTGTGAGTAGGGGGATGAATCAGGGAGGG |
| 198 | GGCGGGGGCAGGGGGCAGGAGCCACACAAACCTGCCCTGGCGAAGACCCC |
| (E14) | CGCTGGCTGACTCAGGGATCTTGCAGCTGTCAGGGGGGAGGGATACAAAT |
| | AGTGCCGACGGCTGGGGGCCCTGTCTCCCCTCGCCGCGGCCGCCACCATG |
| | GCTTCTGCATCAACTTCTAAATATAATTCACACTCCTTGGAGAATGAGTC |
| | TATTAAGAGGACGTCTCGAGATGGAGTCAATCGAGATCTCACTGAGGCTG |
| | TTCCTCGACTTCCAGGAGAAACACTAATCACTGACAAAGAAGTTATTTAC |
| | ATATGTCCTTTCAATGGCCCCATTAAGGGAAGAGTTTACATCACAAATTA |
| | TCGTCTTTATTTAAGAAGTTTGGAAACGGATTCTTCTCTAATACTTGATG |
| | TTCCTCTGGGTGTGATCTCGAGAATTGAAAAAATGGGAGGCGCGACAAGT |
| | AGAGGAGAAAATTCCTATGGTCTAGATATTACTTGTAAAGACATGAGAAA |
| | CCTGAGGTTCGCTTTGAAACAGGAAGGCCACAGCAGAAGAGATATGTTTG |
| | AGATCCTCACGAGATACGCGTTTCCCCTGGCTCACAGTCTGCCATTATTT |
| | GCATTTTTAAATGAAGAAAAGTTTAACGTGGATGGATGGACAGTTTACAA |
| | TCCAGTGGAAGAATACAGGAGGCAGGGCTTGCCCAATCACCATTGGAGAA |
| | TAACTTTTATTAATAAGTGCTATGAGCTCTGTGACACTTACCCTGCTCTT |
| | TTGGTGGTTCCGTATCGTGCCTCAGATGATGACCTCCGGAGAGTTGCAAC |
| | TTTTAGGTCCCGAAATCGAATTCCAGTGCTGTCATGGATTCATCCAGAAA |
| | ATAAGACGGTCATTGTGCGTTGCAGTCAGCCTCTTGTCGGTATGAGTGGG |
| | AAACGAAATAAAGATGATGAGAAATATCTCGATGTTATCAGGGAGACTAA |
| | TAAACAAATTTCTAAACTCACCATTTATGATGCAAGACCCAGCGTAAATG |
| | CAGTGGCCAACAAGGCAACAGGAGGAGGATATGAAAGTGATGATGCATAT |
| | CATAACGCCGAACTTTTCTTCTTAGACATTCATAATATTCATGTTATGCG |
| | GGAATCTTTAAAAAAAGTGAAGGACATTGTTTATCCTAATGTAGAAGAAT |
| | CTCATTGGTTGTCCAGTTTGGAGTCTACTCATTGGTTAGAACATATCAAG |
| | CTCGTTTTGACAGGAGCCATTCAAGTAGCAGACAAAGTTTCTTCAGGGAA |
| | GAGTTCAGTGCTTGTGCATTGCAGTGACGGATGGGACAGGACTGCTCAGC |
| | TGACATCCTTGGCCATGCTGATGTTGGATAGCTTCTATAGGAGCATTGAA |
| | GGGTTCGAAATACTGGTACAAAAAGAATGGATAAGTTTTGGACATAAATT |
| | TGCATCTCGAATAGGTCATGGTGATAAAAACCACACCGATGCTGACCGTT |
| | CTCCTATTTTTCTCCAGTTTATTGATTGTGTGTGGCAAATGTCAAAACAG |
| | TTCCCTACAGCTTTTGAATTCAATGAACAATTTTTGATTATAATTTTGGA |
| | TCATCTGTATAGTTGCCGATTTGGTACTTTCTTATTCAACTGTGAATCTG |
| | CTCGAGAAAGACAGAAGGTTACAGAAAGGACTGTTTCTTTATGGTCACTG |
| | ATAAACAGTAATAAAGAAAAATTCAAAAACCCCTTCTATACTAAAGAAAT |
| | CAATCGAGTTTTATATCCAGTTGCCAGTATGCGTCACTTGGAACTCTGGG |
| | TGAATTACTACATTAGATGGAACCCCAGGATCAAGCAACAACAGCCGAAT |
| | CCAGTGGAGCAGCGTTACATGGAGCTCTTAGCCTTACGCGACGAATACAT |
| | AAAGCGGCTTGAGGAACTGCAGCTCGCCAACTCTGCCAAGCTTTCTGATC |
| | CCCCAACTTCACCTTCCAGTCCTTCGCAAATGATGCCCCATGTGCAAACT |
| | CACTTTTAATTAAGATCTTTTTCCCTCTGCCAAAAATTATGGGGACATCA |
| | TGAAGCCCCTTGAGCATCTGACTTCTGGCTAATAAAGGAAATTTATTTTC |
| | ATTGCAATAGTGTGTTGGAATTTTTTGTGTCTCTCACTCCCTAGGAAAAA |
| | A |
| SEQ ID | TTGGCCACTCCCTCTCTGCGCGCTCGCTCGCTCACTGAGGCCGCCCGGGC |
| NO: | AAAGCCCGGGCGTCGGGCGACCTTTGGTCGCCCGGCCTCAGTGAGCGAGC |
| 199 | GAGCGCGCAGAGAGGGAGTGGCCAACTCCATCACTAGGGGTTCCTTTGTC |
| (15) | GACCCTCTATAAATACCCGCTCTGGGTTGGCAGCTGTTGCTGCGGTGTGT |
| | GTGGGCGTGGGTGGTGTGAGTAGGGGGATGAATCAGGGAGGGGGCGGGGG |
| | CAGGGGGCAGGAGCCACACAAACCTGCCCTGGCGAAGACCCCCGCTGGCT |
| | GACTCAGGGATCTTGCAGCTGTCAGGGGGGAGGGATACAAATAGTGCCGA |
| | CGGCTGGGGGCCCTGTCTCCCCTCGCCGCGGCCGCCACCATGGCTTCTGC |
| | ATCAACTTCTAAATATAATTCACACTCCTTGGAGAATGAGTCTATTAAGA |
| | GGACGTCTCGAGATGGAGTCAATCGAGATCTCACTGAGGCTGTTCCTCGA |
| | CTTCCAGGAGAAACACTAATCACTGACAAAGAAGTTATTTACATATGTCC |
| | TTTCAATGGCCCCATTAAGGGAAGAGTTTACATCACAAATTATCGTCTTT |
| | ATTTAAGAAGTTTGGAAACGGATTCTTCTCTAATACTTGATGTTCCTCTG |
| | GGTGTGATCTCGAGAATTGAAAAAATGGGAGGCGCGACAAGTAGAGGAGA |
| | AAATTCCTATGGTCTAGATATTACTTGTAAAGACATGAGAAACCTGAGGT |
| | TCGCTTTGAAACAGGAAGGCCACAGCAGAAGAGATATGTTTGAGATCCTC |
| | ACGAGATACGCGTTTCCCCTGGCTCACAGTCTGCCATTATTTGCATTTTT |
| | AAATGAAGAAAAGTTTAACGTGGATGGATGGACAGTTTACAATCCAGTGG |
| | AAGAATACAGGAGGCAGGGCTTGCCCAATCACCATTGGAGAATAACTTTT |
| | ATTAATAAGTGCTATGAGCTCTGTGACACTTACCCTGCTCTTTTGGTGGT |
| | TCCGTATCGTGCCTCAGATGATGACCTCCGGAGAGTTGCAACTTTTAGGT |
| | CCCGAAATCGAATTCCAGTGCTGTCATGGATTCATCCAGAAAATAAGACG |
| | GTCATTGTGCGTTGCAGTCAGCCTCTTGTCGGTATGAGTGGGAAACGAAA |
| | TAAAGATGATGAGAAATATCTCGATGTTATCAGGGAGACTAATAAACAAA |
| | TTTCTAAACTCACCATTTATGATGCAAGACCCAGCGTAAATGCAGTGGCC |
| | AACAAGGCAACAGGAGGAGGATATGAAAGTGATGATGCATATCATAACGC |
| | CGAACTTTTCTTCTTAGACATTCATAATATTCATGTTATGCGGGAATCTT |
| | TAAAAAAAGTGAAGGACATTGITTATCCTAATGTAGAAGAATCTCATTGG |
| | TTGTCCAGTTTGGAGTCTACTCATTGGTTAGAACATATCAAGCTCGTTTT |
| | GACAGGAGCCATTCAAGTAGCAGACAAAGTTTCTTCAGGGAAGAGTTCAG |
| | TGCTTGTGCATTGCAGTGACGGATGGGACAGGACTGCTCAGCTGACATCC |
| | TTGGCCATGCTGATGTTGGATAGCTTCTATAGGAGCATTGAAGGGTTCGA |
| | AATACTGGTACAAAAAGAATGGATAAGTTTTGGACATAAATTTGCATCTC |
| | GAATAGGTCATGGTGATAAAAACCACACCGATGCTGACCGTTCTCCTATT |
| | TTTCTCCAGTTTATTGATTGTGTGTGGCAAATGTCAAAACAGTTCCCTAC |
| | AGCTTTTGAATTCAATGAACAATTTTTGATTATAATTTTGGATCATCTGT |
| | ATAGTTGCCGATTTGGTACTTTCTTATTCAACTGTGAATCTGCTCGAGAA |
| | AGACAGAAGGTTACAGAAAGGACTGTTTCTTTATGGTCACTGATAAACAG |
| | TAATAAAGAAAAATTCAAAAACCCCTTCTATACTAAAGAAATCAATCGAG |
| | TTTTATATCCAGTTGCCAGTATGCGTCACTTGGAACTCTGGGTGAATTAC |
| | TACATTAGATGGAACCCCAGGATCAAGCAACAACAGCCGAATCCAGTGGA |
| | GCAGCGTTACATGGAGCTCTTAGCCTTACGCGACGAATACATAAAGCGGC |
| | TTGAGGAACTGCAGCTCGCCAACTCTGCCAAGCTTTCTGATCCCCCAACT |
| | TCACCTTCCAGTCCTTCGCAAATGATGCCCCATGTGCAAACTCACTTTTA |
| | ATTAAGATCTTTTTCCCTCTGCCAAAAATTATGGGGACATCATGAAGCCC |
| | CTTGAGCATCTGACTTCTGGCTAATAAAGGAAATTTATTTTCATTGCAAT |
| | AGTGTGTTGGAATTTTTTGTGTCTCTCACTCGCCTAGGCCACTCCCTCTC |
| | TGCGCGCTCGCTCGCTCACTGAGGCCGGGCGACCAAAGGTCGCCCGACGC |
| | CCGGGCTTTGCCCGGGCGGCCTCAGTGAGCGAGCGAGCGCGCAGAGAGGG |
| | AGTGGCCAA |
| SEQ ID | TTTTGGTACCGACATTGATTATTGACTAGTTATTAATAGTAATCAATTAC |
| NO: | GGGGTCATTAGTTCATAGCCCATATATGGAGTTCCGCGTTACATAACTTA |
| 200 | CGGTAAATGGCCCGCCTGGCTGACCGCCCAACGACCCCCGCCCATTGACG |
| (E16) | TCAATAATGACGTATGTTQCCATAGTAACGCCAATAGGGACTTTCCATTG |
| | ACGTCAATGGGTGGAGTATTTACGGTAAACTGCCCACTTGGCAGTACATC |
| | AAGTGTATCATATGCCAAGTACGCCCCCTATTGACGTCAATGACGGTAAA |
| | TGGCCCGCCTGGCATTATGCCCAGTACATGACCTTATGGGACTTTCCTAC |
| | TTGGCAGTACATCTACGTATTAGTCATCGCTATTACCATGGTCGAGGTGA |
| | GCCCCACGTTCTGCTTCACTCTCCCCATCTCCCCCCCCTCCCCACCCCCA |
| | ATTTTGTATTTATTTATTTTTTAATTATTTTGTGCAGCGATGGGGGGGGG |
| | GGGGGGGGGGGGGGGCGCGCCAGGCGGGGCGGGGCGGGGCGAGGGGGGGG |
| | GGGGGGCGAGGCGGAGAGGTGCGGCGGCAGCCAATCAGAGCGGCGCGCTC |
| | CGAAAGTTTCCTTTTATGGCGAGGCGGCGGCGGCGGCGGCCCTATAAAAA |
| | GCGAAGCGCGCGGCGGGCGGATATCTTTTTT |
| SEQ ID | TTGGCCACTCCCTCTCTGCGCGCTCGCTCGCTCACTGAGGCCGCCCGGGC |
| NO: | AAAGCCCGGGCGTCGGGCGACCTTTGGTCGCCCGGCCTCAGTGAGCGAGC |
| 201 | GAGCGCGCAGAGAGGGAGTGGCCAACTCCATCACTAGGGGTTCCTTTGTC |
| (E17) | GACGACATTGATTATTGACTAGTTATTAATAGTAATCAATTACGGGGTCA |
| | TTAGTTCATAGCCCATATATGGAGTTCCGCGTTACATAACTTACGGTAAA |
| | TGGCCCGCCTGGCTGACCGCCCAACGACCCCCGCCCATTGACGTCAATAA |
| | TGACGTATGTTCCCATAGTAACGCCAATAGGGACTTTCCATTGACGTCAA |
| | TGGGTGGAGTATTTACGGTAAACTGCCCACTTGGCAGTACATCAAGTGTA |
| | TCATATGCCAAGTACGCCCCCTATTGACGTCAATGACGGTAAATGGCCCG |
| | CCTGGCATTATGCCCAGTACATGACCTTATGGGACTTTCCTACTTGGCAG |
| | TACATCTACGTATTAGTCATCGCTATTACCATGGTCGAGGTGAGCCCCAC |
| | GTTCTGCTTCACTCTCCCCATCTCCCCCCCCTCCCCACCCCCAATTTTGT |
| | ATTTATTTATTTTTTAATTATTTTGTGCAGCGATGGGGGCGGGGGGGGGG |
| | GGGGGGCGCGCGCCAGGCGGGGCGGGGCGGGGCGAGGGGGGGGGGGGGCG |
| | AGGCGGAGAGGTGCGGCGGCAGCCAATCAGAGCGGCGCGCTCCGAAAGTT |
| | TCCTTTTATGGCGAGGCGGCGGCGGCGGCGGCCCTATAAAAAGCGAAGCG |
| | CGCGGGGGCGGGAGTGGCTGCGCGCTGCCTTCGCCCCGTGCCCCGCTCCG |
| | CCGCCGCCTCGCGCCGCCCGCCCCGGCTCTGACTGACCGCGTTACTCCCA |
| | CAGGTGAGCGGGCGGGACGGCCCTTCTCCTCCGGGCTGTAATTAGCGCTT |
| | GGTTTAATGACGGCTTGTTTCTTTTCTGTGGCTGCGTGAAAGCCTTGAGG |
| | GGCTCCGGGAGGGCCCTTTGTGCGGGGGGAGCGGCTCGGGGGGTGCGTGC |
| | GTGTGTGTGTGCGTGGGGAGCGCCGCGTGCGGCTCCGCGCTGCCCGGCGG |
| | CTGTGAGCGCTGCGGGCGCGGCGCGGGGCTTTGTGCGCTCCGCAGTGTGC |
| | GCGAGGGGAGCGCGGCCGGGGGCGGTGCCCCGCGGTGCGGGGGGGGCTGC |
| | GAGGGGAACAAAGGCTGCGTGCGGGGTGTGTGCGTGGGGGGGTGAGCAGG |
| | GGGTGTGGGCGCGTCGGTCGGGCTGCAACCCCCCCTGCACCCCCCTCCCC |
| | GAGTTGCTGAGCACGGCCCGGCTTCGGGTGCGGGGCTCCGTACGGGGCGT |
| | GGCGCGGGGCTCGCCGTGCCGGGGGGGGGGTGGCGGCAGGTGGGGGTGCC |
| | GGGGGGGGGGGGCCGCCTCGGGCCGGGGAGGGCTCGGGGGAGGGGCGCGG |
| | CGGCCCCCGGAGCGCCGGCGGCTGTCGAGGCGCGGCGAGCCGCAGCCATT |
| | GCCTTTTATGGTAATCGTGCGAGAGGGCGCAGGGACTTCCTTTGTCCCAA |
| | ATCTGTGCGGAGCCGAAATCTGGGAGGCGCCGCCGCACCCCCTCTAGCGG |
| | GCGCGGGGCGAAGCGGTGCGGCGCCGGCAGGAAGGAAATGGGGGGGGAGG |
| | GCCTTCGTGCGTCGCCGCGCCGCCGTCCCCTTCTCCCTCTCCAGCCTCGG |
| | GGCTGTCCGCGGGGGGACGGCTGCCTTCGGGGGGGACGGGGCAGGGGGGG |
| | GTTCGGCTTCTGGCGTGTGACCGGCGGCTCTAGAGCCTCTGCTAACCATG |
| | TTCATGCCTTCTTCTTTTTCCTACAGCTCCTGGGCAACGTGCTGGTTATT |
| | GTGCTGTCTCATCATTTTGGCAAAAGATCTGCCGCCACCATGGTGAGCAA |
| | GGGCGAGGAGCTGTTCACCGGGGTGGTGCCCATCCTGGTCGAGCTGGACG |
| | GCGACGTAAACGGCCACAAGITCAGCGTGTCCGGCGAGGGCGAGGGCGAT |
| | GCCACCTACGGCAAGCTGACCCTGAAGTTCATCTGCACCACCGGCAAGCT |
| | GCCCGTGCCCTGGCCCACCCTCGTGACCACCCTGACCTACGGCGTGCAGT |
| | GCTTCAGCCGCTACCCCGACCACATGAAGCAGCACGACTTCTTCAAGTCC |
| | GCCATGCCCGAAGGCTACGTCCAGGAGCGCACCATCTTCTTCAAGGACGA |
| | CGGCAACTACAAGACCCGCGCCGAGGTGAAGTTCGAGGGCGACACCCTGG |
| | TGAACCGCATCGAGCTGAAGGGCATCGACTTCAAGGAGGACGGCAACATC |
| | CTGGGGCACAAGCTGGAGTACAACTACAACAGCCACAACGTCTATATCAT |
| | GGCCGACAAGCAGAAGAACGGCATCAAGGTGAACTTCAAGATCCGCCACA |
| | ACATCGAGGACGGCAGCGTGCAGCTCGCCGACCACTACCAGCAGAACACC |
| | CCCATCGGCGACGGCCCCGTGCTGCTGCCCGACAACCACTACCTGAGCAC |
| | CCAGTCCGCCCTGAGCAAAGACCQCAACGAGAAGCGCGATCACATGGTCC |
| | TGCTGGAGTTCGTGACCGCCGCCGGGATCACTCTCGGCATGGACGAGCTG |
| | TACAAGTGAGAGCTCGATCTTTTTCCCTCTGCCAAAAATTATGGGGACAT |
| | CATGAAGCCCCTTGAGCATCTGACTTCTGGCTAATAAAGGAAATTTATTT |
| | TCATTGCAATAGTGTGTTGGAATTTTTTGTGTCTCTCACTCGGAAGCCTA |
| | GGAGGAACCCCTAGTGATGGAGTTGGCCACTCCCTCTCTGCGCGCTCGCT |
| | CGCTCACTGAGGCCGGGCGACCAAAGGTCGCCCGACGCCCGGGCTTTGCC |
| | CGGGGGCCTCAGTGAGCGAGCGAGCGCGCAGAGAGGGAGTGGCCAA |
| SEQ ID | CAAAGAATTTGAGCGGCCGCCACCATGGCTTCTGCATCAACTTCTAAATA |
| NO: | TAATTCACACTCCTTGGAGAATGAGTCTATTAAGAGGACGTCTCGAGATG |
| 202 | GAGTCAATCGAGATCTCACTGAGGCTGTTCCTCGACTTCCAGGAGAAACA |
| (E18) | CTAATCACTGACAAAGAAGTTATTTACATATGTCCTTTCAATGGCCCCAT |
| | TAAGGGAAGAGTTTACATCACAAATTATCGTCTTTATTTAAGAAGTTTGG |
| | AAACGGATTCTTCTCTAATACTTGATGTTCCTCTGGGTGTGATCTCGAGA |
| | ATTGAAAAAATGGGAGGCGCGACAAGTAGAGGAGAAAATTCCTATGGTCT |
| | AGATATTACTTGTAAAGACATGAGAAACCTGAGGTTCGCTTTGAAACAGG |
| | AAGGCCACAGCAGAAGAGATATGTTTGAGATCCTCACGAGATACGCGTTT |
| | CCCCTGGCTCACAGTCTGCCATTATTTGCATTTTTAAATGAAGAAAAGTT |
| | TAACGTGGATGGATGGACAGTTTACAATCCAGTGGAAGAATACAGGAGGC |
| | AGGGCTTGCCCAATCACCATTGGAGAATAACTTTTATTAATAAGTGCTAT |
| | GAGCTCTGTGACACTTACCCTGCTCTTTTGGTGGTTCCGTATCGTGCCTC |
| | AGATGATGACCTCCGGAGAGTTGCAACTTTTAGGTCCCGAAATCGAATTC |
| | CAGTGCTGTCATGGATTCATCCAGAAAATAAGACGGTCATTGTGCGTTGC |
| | AGTCAGCCTCTTGTCGGTATGAGTGGGAAACGAAATAAAGATGATGAGAA |
| | ATATCTCGATGTTATCAGGGAGACTAATAAACAAATTTCTAAACTCACCA |
| | TTTATGATGCAAGACCCAGCGTAAATGCAGTGGCCAACAAGGCAACAGGA |
| | GGAGGATATGAAAGTGATGATGCATATCATAACGCCGAACTTTTCTTCTT |
| | AGACATTCATAATATTCATGTTATGCGGGAATCTTTAAAAAAAGTGAAGG |
| | ACATTGTTTATCCTAATGTAGAAGAATCTCATTGGTTGTCCAGTTTGGAG |
| | TCTACTCATTGGTTAGAACATATCAAGCTCGTTTTGACAGGAGCCATTCA |
| | AGTAGCAGACAAAGTTTCTTCAGGGAAGAGTTCAGTGCTTGTGCATTGCA |
| | GTGACGGATGGGACAGGACTGCTCAGCTGACATCCTTGGCCATGCTGATG |
| | TTGGATAGCTTCTATAGGAGCATTGAAGGGTTCGAAATACTGGTACAAAA |
| | AGAATGGATAAGTTTTGGACATAAATTTGCATCTCGAATAGGTCATGGTG |
| | ATAAAAACCACACCGATGCTGACCGTTCTCCTATTTTTCTCCAGTTTATT |
| | GATTGTGTGTGGCAAATGTCAAAACAGTTCCCTACAGCTTTTGAATTCAA |
| | TGAACAATTTTTGATTATAATTTTGGATCATCTGTATAGITGCCGATTTG |
| | GTACTTTCTTATTCAACTGTGAATCTGCTCGAGAAAGACAGAAGGTTACA |
| | GAAAGGACTGTTTCTTTATGGTCACTGATAAACAGTAATAAAGAAAAATT |
| | CAAAAACCCCTTCTATACTAAAGAAATCAATCGAGTTTTATATCCAGTTG |
| | CCAGTATGCGTCACTTGGAACTCTGGGTGAATTACTACATTAGATGGAAC |
| | CCCAGGATCAAGCAACAACAGCCGAATCCAGTGGAGCAGCGTTACATGGA |
| | GCTCTTAGCCTTACGCGACGAATACATAAAGCGGCTTGAGGAACTGCAGC |
| | TCGCCAACTCTGCCAAGCTTTCTGATCCCCCAACTTCACCTTCCAGTCCT |
| | TCGCAAATGATGCCCCATGTGCAAACTCACTTTTAATTAAGATCTTTTT |
| SEQ ID | TTTGAGCGGCCGCCACCATGGCTTCTGCATCAACTTCTAAATATAATTCA |
| NO: | CACTCCTTGGAGAATGAGTCTATTAAGAGGACGTCTCGAGATGGAGTCAA |
| 203 | TCGAGATCTCACTGAGGCTGTTCCTCGACTTCCAGGAGAAACACTAATCA |
| (E19) | CTGACAAAGAAGTTATTTACATATGTCCTTTCAATGGCCCCATTAAGGGA |
| | AGAGTTTACATCACAAATTATCGTCTTTATTTAAGAAGTTTGGAAACGGA |
| | TTCTTCTCTAATACTTGATGTTCCTCTGGGTGTGATCTCGAGAATTGAAA |
| | AAATGGGAGGCGCGACAAGTAGAGGAGAAAATTCCTATGGTCTAGATATT |
| | ACTTGTAAAGACATGAGAAACCTGAGGTTCGCTTTGAAACAGGAAGGCCA |
| | CAGCAGAAGAGATATGTTTGAGATCCTCACGAGATACGCGTTTCCCCTGG |
| | CTCACAGTCTGCCATTATTTGCATTTTTAAATGAAGAAAAGTTTAACGTG |
| | GATGGATGGACAGTTTACAATCCAGTGGAAGAATACAGGAGGCAGGGCTT |
| | GCCCAATCACCATTGGAGAATAACTTTTATTAATAAGTGCTATGAGCTCT |
| | GTGACACTTACCCTGCTCTTTTGGTGGTTCCGTATCGTGCCTCAGATGAT |
| | GACCTCCGGAGAGTTGCAACTTTTAGGTCCCGAAATCGAATTCCAGTGCT |
| | GTCATGGATTCATCCAGAAAATAAGACGGTCATTGTGCGTTGCAGTCAGC |
| | CTCTTGTQGGTATGAGTGGGAAACGAAATAAAGATGATGAGAAATATCTC |
| | GATGTTATCAGGGAGACTAATAAACAAATTTCTAAACTCACCATTTATGA |
| | TGCAAGACCCAGCGTAAATGCAGTGGCCAACAAGGCAACAGGAGGAGGAT |
| | ATGAAAGTGATGATGCATATCATAACGCCGAACTTTTCTTCTTAGACATT |
| | CATAATATTCATGTTATGCGGGAATCTTTAAAAAAAGTGAAGGACATTGT |
| | TTATCCTAATGTAGAAGAATCTCATTGGITGTCCAGTTTGGAGTCTACTC |
| | ATTGGTTAGAACATATCAAGCTCGTTTTGACAGGAGCCATTCAAGTAGCA |
| | GACAAAGTTTCTTCAGGGAAGAGTTCAGTGCTTGTGCATTGCAGTGACGG |
| | ATGGGACAGGACTGCTCAGCTGACATCCTTGGCCATGCTGATGTTGGATA |
| | GCTTCTATAGGAGCATTGAAGGGTTCGAAATACTGGTACAAAAAGAATGG |
| | ATAAGTTTTGGACATAAATTTGCATCTCGAATAGGTCATGGTGATAAAAA |
| | CCACACCGATGCTGACCGTTCTCCTATTTTTCTCCAGTTTATTGATTGTG |
| | TGTGGCAAATGTCAAAACAGTTCCCTACAGCTTTTGAATTCAATGAACAA |
| | TTTTTGATTATAATTTTGGATCATCTGTATAGTTGCCGATTTGGTACTTT |
| | CTTATTCAACTGTGAATCTGCTCGAGAAAGACAGAAGGTTACAGAAAGGA |
| | CTGTTTCTTTATGGTCACTGATAAACAGTAATAAAGAAAAATTCAAAAAC |
| | CCCTTCTATACTAAAGAAATCAATCGAGTTTTATATCCAGTTGCCAGTAT |
| | GCGTCACTTGGAACTCTGGGTGAATTACTACATTAGATGGAACCCCAGGA |
| | TCAAGCAACAACAGCCGAATCCAGTGGAGCAGCGTTACATGGAGCTCTTA |
| | GCCTTACGCGACGAATACATAAAGCGGCTTGAGGAACTGCAGCTCGCCAA |
| | CTCTGCCAAGCTTTCTGATCCCCCAACTTCACCTTCCAGTCCTTCGCAAA |
| | TGATGCCCCATGTGCAAACTCACTTTTAATTAAGATC |
| SEQ ID | TTGGCCACTCCCTCTCTGCGCGCTCGCTCGCTCACTGAGGCCGCCCGGGC |
| NO: | AAAGCCCGGGCGTCGGGCGACCTTTGGTCGCCCGGCCTCAGTGAGCGAGC |
| 204 | GAGCGCGCAGAGAGGGAGTGGCCAACTCCATCACTAGGGGTTCCTTGCGC |
| (E20A) | ACAGGTACCCCCCTGCCCCCACAGCTCCTCTCCTGTGCCTTGTTTCCCAG |
| | CCATGCGTTCTCCTCTATAAATACCCGCTCTGGTATTTGGGGTTGGCAGC |
| | TGTTGCTGCCAGGGAGATGGTTGGGTTGACATGCGGCTCCTGACAAAACA |
| | CAAACCCCTGGTGTGTGTGGGCGTGGGTGGTGTGAGTAGGGGGATGAATC |
| | AGGGAGGGGGCGGGGGACCCAGGGGGCAGGAGCCACACAAAGTCTGTGCG |
| | GGGGTGGGAGCGCACATAGCAATTGGAAACTGGCTGCAGACATGCTTGCT |
| | GCCTGCCCTGGCGAAGGATTGGTAGGCTTGCCGTCACAGGACCCCCGCTG |
| | GCTGACTCAGGGGCGCAGGCTCTTGCGGGGGAGCTGGCCTCCCGCCCCCA |
| | CGGCCACGGGCCCTTTCCTGGCAGGACAGCGGGATCTTGCAGCTGTCAGG |
| | GGAGGGGAGGCGGGGGCTGATGTCAGGAGGGATACAAATAGTGCCGACGG |
| | CTGGGGGCCCTGTCTCCCCTCGCCGCATCCACTCTCCGGCCGGCCGCCTG |
| | CCCGCCGCCTCCTCCGTGCGCCCGCCAGCCTCGCCCGCGCCGTCACCGAT |
| | ATCTCAGATCGCCTGGAGACGCCATCCACGCTGTTTTGACCTCCATAGAA |
| | GACACCGGGACCGATCCAGCCTCCGCGGATTCGAATCCCGGCCGGGAACG |
| | GTGCATTGGAACGCGGATTCCCCGTGCCAAGAGTGACGTAAGTACCGCCT |
| | ATAGAGTCTATAGGCCCACAAAAAATGCTTTCTTCTTTTAATATACTTTT |
| | TTGTTTATCTTATTTCTAATACTTTCCCTAATCTCTTTCTTTCAGGGCAA |
| | TAATGATACAATGTATCATGCCTCTTTGCACCATTCTAAAGAATAACAGT |
| | GATAATTTCTGGGTTAAGGCAATAGCAATATTTCTGCATATAAATATTTC |
| | TGCATATAAATTGTAACTGATGTAAGAGGTTTCATATTGCTAATAGCAGC |
| | TACAATCCAGCTACCATTCTGCTTTTATTTTATGGTTGGGATAAGGCTGG |
| | ATTATTCTGAGTCCAAGCTAGGCCCTTTTGCTAATCATGTTCATACCTCT |
| | TATCTTCCTCCCACAGCTCCTGGGCAACGTGCTGGTCTGTGTGCTGGCCC |
| | ATCACTTTGGCAAAGAATTGGATCCGCCGCCACC |
| SEQ ID | TAAGTCGACGTAAGTTTTTAAATGTATAAATTGTCTTATTTATAAATTGG |
| NO: | TCTAAAATATATGTAATTGTCTTAAGATCTTTTTCCCTCTGCCAAAAATT |
| 205 | ATGGGGACATCATGAAGCCCCTTGAGCATCTGACTTCTGGCTAATAAAGG |
| (E20B) | AAATTTATTTTCATTGCAATAGTGTGTTGGAATTTTTTGTGTCTCTCACT |
| | CGAGGCCTAGGAACCCCTAGTGATGGAGTTGGCCACTCCCTCTCTGCGCG |
| | CTCGCTCGCTCACTGAGGCCGGGCGACCAAAGGTCGCCCGACGCCCGGGC |
| | TTTGCCCGGGCGGCCTCAGTGAGCGAGCGAGCGCGCAGAGAGGGAGTGGC |
| | CAA |
| SEQ ID | TTGGCCACTCCCTCTCTGCGCGCTCGCTCGCTCACTGAGGCCGCCCGGGC |
| NO: | AAAGCCCGGGCGTCGGGCGACCTTTGGTCGCCCGGCCTCAGTGAGCGAGC |
| 206 | GAGCGCGCAGAGAGGGAGTGGCCAACTCCATCACTAGGGGTTCCTTTGTC |
| (E21A) | GACTCGGTTCGCATATTAAGGTGACGCGTGTGGCCTCGAACACCGAGCGA |
| | CCCTGCAGCGACCCGCTTAA |
| SEQ ID | AATAAAGGAAATTTATTTTCATTGCAATAGTGTGTTGGAATTTTTTGTGT |
| NO: | CTCTCAGAGTCCTAGGCCACTCCCTCTCTGCGCGCTCGCTCGCTCACTGA |
| 207 | GGCCGGGCGACCAAAGGTCGCCCGACGCCCGGGCTTTGCCCGGGCGGCCT |
| (E21B) | CAGTGAGCGAGCGAGCGCGCAGAGAGGGAGTGGCCAA |
| SEQ ID | TTGGCCACTCCCTCTCTGCGCGCTCGCTCGCTCACTGAGGCCGCCCGGGC |
| NO: | AAAGCCCGGGCGTCGGGCGACCTTTGGTCGCCCGGCCTCAGTGAGCGAGC |
| 208 | GAGCGCGCAGAGAGGGAGTGGCCAACTCCATCACTAGGGGTTCCTTTGTC |
| (E22) | GACTTCGCATATTAAGGTGACGCGTGTGGCCTCGAACACCGAGCGACCCT |
| | GCAGCGACCCGCTTAAGCGGCCGCCACCATGGCTTCTGCATCAACTTCTA |
| | AATATAATTCACACTCCTTGGAGAATGAGTCTATTAAGAGGACGTCTCGA |
| | GATGGAGTCAATCGAGATCTCACTGAGGCTGTTCCTCGACTTCCAGGAGA |
| | AACACTAATCACTGACAAAGAAGTTATTTACATATGTCCTTTCAATGGCC |
| | CCATTAAGGGAAGAGTTTACATCACAAATTATCGTCTTTATTTAAGAAGT |
| | TTGGAAACGGATTCTTCTCTAATACTTGATGTTCCTCTGGGTGTGATCTC |
| | GAGAATTGAAAAAATGGGAGGCGCGACAAGTAGAGGAGAAAATTCCTATG |
| | GTCTAGATATTACTTGTAAAGACATGAGAAACCTGAGGTTCGCTTTGAAA |
| | CAGGAAGGCCACAGCAGAAGAGATATGTTTGAGATCCTCACGAGATACGC |
| | GTTTCCCCTGGCTCACAGTCTGCCATTATTTGCATTTTTAAATGAAGAAA |
| | AGTTTAACGTGGATGGATGGACAGTTTACAATCCAGTGGAAGAATACAGG |
| | AGGCAGGGCTTGCCCAATCACCATTGGAGAATAACTTTTATTAATAAGTG |
| | CTATGAGCTCTGTGACACTTACCCTGCTCTTTTGGTGGTTCCGTATCGTG |
| | CCTCAGATGATGACCTCCGGAGAGTTGCAACTTTTAGGTCCCGAAATCGA |
| | ATTCCAGTGCTGTCATGGATTCATCCAGAAAATAAGACGGTCATTGTGCG |
| | TTGCAGTCAGCCTCTTGTCGGTATGAGTGGGAAACGAAATAAAGATGATG |
| | AGAAATATCTCGATGTTATCAGGGAGACTAATAAACAAATTTCTAAACTC |
| | ACCATTTATGATGCAAGACCCAGCGTAAATGCAGTGGCCAACAAGGCAAC |
| | AGGAGGAGGATATGAAAGTGATGATGCATATCATAACGCCGAACTTTTCT |
| | TCTTAGACATTCATAATATTCATGTTATGCGGGAATCTTTAAAAAAAGTG |
| | AAGGACATTGTTTATCCTAATGTAGAAGAATCTCATTGGTTGTCCAGTTT |
| | GGAGTCTACTCATTGGTTAGAACATATCAAGCTCGTTTTGACAGGAGCCA |
| | TTCAAGTAGCAGACAAAGTTTCTTCAGGGAAGAGTTCAGTGCTTGTGCAT |
| | TGCAGTGACGGATGGGACAGGACTGCTCAGCTGACATCCTTGGCCATGCT |
| | GATGTTGGATAGCTTCTATAGGAGCATTGAAGGGTTCGAAATACTGGTAC |
| | AAAAAGAATGGATAAGTTTTGGACATAAATTTGCATCTCGAATAGGTCAT |
| | GGTGATAAAAACCACACCGATGCTGACCGTTCTCCTATTTTTCTCCAGTT |
| | TATTGATTGTGTGTGGCAAATGTCAAAACAGTTCCCTACAGCTTTTGAAT |
| | TCAATGAACAATTTTTGATTATAATTTTGGATCATCTGTATAGTTGCCGA |
| | TTTGGTACTTTCTTATTCAACTGTGAATCTGCTCGAGAAAGACAGAAGGT |
| | TACAGAAAGGACTGTTTCTTTATGGTCACTGATAAACAGTAATAAAGAAA |
| | AATTCAAAAACCCCTTCTATACTAAAGAAATCAATCGAGTTTTATATCCA |
| | GTTGCCAGTATGCGTCACTTGGAACTCTGGGTGAATTACTACATTAGATG |
| | GAACCCCAGGATCAAGCAACAACAGCCGAATCCAGTGGAGCAGCGTTACA |
| | TGGAGCTCTTAGCCTTACGCGACGAATACATAAAGCGGCTTGAGGAACTG |
| | CAGCTCGCCAACTCTGCCAAGCTTTCTGATCCCCCAACTTCACCTTCCAG |
| | TCCTTCGCAAATGATGCCCCATGTGCAAACTCACTTTTAATTAAGATCTT |
| | TTTCCCTCTGCCAAAAATTATGGGGACATCATGAAGCCCCTTGAGCATCT |
| | GACTTCTGGCTAATAAAGGAAATTTATTTTCATTGCAATAGTGTGTTGGA |
| | ATTTTTTGTGTCTCTCACTCCCTAGGCCACTCCCTCTCTGCGCGCTCGCT |
| | CGCTCACTGAGGCCGGGCGACCAAAGGTCGCCCGACGCCCGGGCTTTGCC |
| | CGGGCGGCCTCAGTGAGCGAGCGAGCGCGCAGAGAGGGAGTGGCCAA |
| SEQ ID | ttttGGTACCgacattgattattgactagttatt |
| NO: | |
| 209 | |
| SEQ ID | aaaaaa gatatc cgcocgocgogc |
| NO: | |
| 210 | |
| SEQ ID | TttttGtcGACTTCGCATATTAAGGTGACGCGT |
| NO: | |
| 211 | |
| SEQ ID | tttttt cctagg gagTGAGAGACACAAAAAATTCCAACACAC |
| NO: | |
| 212 | |
| SEQ ID | tttttGtcGACCCTCTATAAATACCCGCTCTGG |
| NO: | |
| 213 | |
| SEQ ID | tttttt cctagg gagTGAGAGACACAAAAAATTCCAACACAC |
| NO: | |
| 214 | |
| Anc80- | MAADGYLPDWLEDNLSEGIREWWDLKPGAPKPKANQQKQDDGRGLVLPGY |
| 55 | KYLGPFNGLDKGEPVNAADAAALEHDKAYDQQLKAGDNPYLRYNHADAEF |
| SEQ ID | QERLQEDTSFGGNLGRAVFQAKKRVLEPLGLVEEGAKTAPGKKRPVEQSP |
| NO: | QEPDSSSGIGKKGQQPAKKRLNFGQTGDSESVPDPQPLGEPPAAPSGVGS |
| 44885 | NTMAAGGGAPMADNNEGADGVGNASGNWHCDSTWLGDRVITTSTRTWALP |
| | TYNNHLYKQISSQSGASTNDNTYFGYSTPWGYFDFNRFHCHFSPRDWQRL |
| | INNNWGFRPKKLNFKLFNIQVKEVTINDGTTTIANNLTSTVQVFTDSEYQ |
| | LPYVLGSAHQGCLPPFPADVFMIPQYGYLTLNNGSQAVGRSSFYCLEYFP |
| | SQMLRTGNNFQFSYTFEDVPFHSSYAHSQSLDRLMNPLIDQYLYYLSRTQ |
| | TTSGTAGNRELQFSQAGPSSMANQAKNWLPGPCYRQQRVSKTANQNNNSN |
| | FAWTGATKYHLNGRDSLVNPGPAMATHKDDEDKFFPMSGVLIFGKQGAGN |
| | SNVDLDNVMITNEEEIKTTNPVATEEYGTVATNLQSSNTAPATGTVNSQG |
| | ALPGMVWQDRDVYLQGPIWAKIPHTDGHFHPSPLMGGFGLKHPPPQILIK |
| | NTPVPANPPTTFSPAKFASFITQYSTGQVSVEIEWELQKENSKRWNPEIQ |
| | YTSNYNKSTNVDFAVDINGVYSEPRPIGTRYLTRNL |
| Anc80- | MAADGYLPDWLEDNLSEGIREWWDLKPGAPKPKANQQKQDDGRGLVLPGY |
| 55 - | KYLGPFNGLDKGEPVNAADAAALEHDKAYDQQLKAGDNPYLRYNHADAEF |
| decol | QERLQEDTSFGGNLGRAVFQAKKRVLEPLGLVEEGAKTAPGKKRPVEQSP |
| SEQ ID | QEPDSSSGIGKKGQQPAKKRLNFGQTGDSESVPDPQPLGEPPAAPSGVGS |
| NO: | NTMAAGGGAPMADNNEGADGVGNASGNWHCDSTWLGDRVITTSTRTWALP |
| 44886 | TYNNHLYKQISSQSGASTNDNTYFGYSTPWGYFDFNRFHCHFSPRDWQRL |
| | INNNWGFRPKKLNFKLFNIQVKEVTINDGTTTIANNLTSTVQVFTDSEYQ |
| | LPYVLGSAHQGCLPPFPADVFMIPQYGYLTLNNGSQAVGRSSFYCLEYFP |
| | SQMLRTGNNFQFSYTFEDVPFHSSYAHSQSLDRLMNPLIDQYLYYLSRTQ |
| | TTSGTAGNRELQFSQAGPSSMANQAKNWLPGPCYRQQRVSKTANQNNNSN |
| | FAWTGATKYHLNGRDSLVNPGPAMATHKDDEDKFFPMSGVLIFGKQGAGN |
| | SNVDLDNVMITNEEEIKTINPVATEEYGTVATNLQSSNRGDLLLSTAPAT |
| | GTVNSQGALPGMVWQDRDVYLQGPIWAKIPHTDGHFHPSPLMGGFGLKHP |
| | PPQILIKNTPVPANPPTTFSPAKFASFITQYSTGQVSVEIEWELQKENSK |
| | RWNPEIQYTSNYNKSTNVDFAVDINGVYSEPRPIGTRYLTRNL |
| Anc80 | MAADGYLPDWLEDNLSEGIREWWDLKPGAPKPKANQQKQDDGRGLVLPGY |
| 129 | KYLGPFNGLDKGEPVNAADAAALEHDKAYDQQLKAGDNPYLRYNHADAEF |
| SEQ ID | QERLQEDTSFGGNLGRAVFQAKKRVLEPLGLVEEGAKTAPGKKRPVEQSP |
| NO: | QEPDSSSGIGKKGQQPAKKRLNFGQTGDSESVPDPQPLGEPPAAPSGVGS |
| 44887 | NTMAAGGGAPMADNNEGADGVGNASGNWHCDSTWLGDRVITTSTRTWALP |
| | TYNNHLYKQISSQSGASTNDNTYFGYSTPWGYFDFNRFHCHFSPRDWQRL |
| | INNNWGFRPKRLNFKLFNIQVKEVTINDGTTTIANNLTSTVQVFTDSEYQ |
| | LPYVLGSAHQGCLPPFPADVFMIPQYGYLTLNNGSQAVGRSSFYCLEYFP |
| | SQMLRTGNNFQFSYTFEDVPFHSSYAHSQSLDRLMNPLIDQYLYYLSRTQ |
| | TTSGTAGNRTLQFSQAGPSSMANQAKNWLPGPCYRQQRVSKTTNQNNNSN |
| | FAWTGATKYHLNGRDSLVNPGPAMATHKDDEDKFFPMSGVLIFGKQGAGN |
| | SNVDLDNVMITNEEEIKTTNPVATEQYGTVATNLQSANTAPATGTVNSQG |
| | ALPGMVWQDRDVYLQGPIWAKIPHTDGHFHPSPLMGGFGLKHPPPQILIK |
| | NTPVPANPPTTFSPAKFASFITQYSTGQVSVEIEWELQKENSKRWNPEIQ |
| | YTSNYNKSTNVDFAVDINGVYSEPRPIGTRYLTRNL |
| Anc80- | MAADGYLPDWLEDNLSEGIREWWDLKPGAPKPKANQQKQDDGRGLVLPGY |
| 129- | KYLGPFNGLDKGEPVNAADAAALEHDKAYDQQLKAGDNPYLRYNHADAEF |
| decol | QERLQEDTSFGGNLGRAVFQAKKRVLEPLGLVEEGAKTAPGKKRPVEQSP |
| SEQ ID | QEPDSSSGIGKKGQQPAKKRLNFGQTGDSESVPDPQPLGEPPAAPSGVGS |
| NQ | NTMAAGGGAPMADNNEGADGVGNASGNWHCDSTWLGDRVITTSTRTWALP |
| 44888 | TYNNHLYKQISSQSGASTNDNTYFGYSTPWGYFDFNRFHCHFSPRDWQRL |
| | INNNWGFRPKRLNFKLFNIQVKEVTINDGTTTIANNLTSTVQVFTDSEYQ |
| | LPYVLGSAHQGCLPPFPADVFMIPQYGYLTLNNGSQAVGRSSFYCLEYFP |
| | SQMLRTGNNFQFSYTFEDVPFHSSYAHSQSLDRLMNPLIDQYLYYLSRTQ |
| | TTSGTAGNRTLQFSQAGPSSMANQAKNWLPGPCYRQQRVSKTTNQNNNSN |
| | FAWTGATKYHLNGRDSLVNPGPAMATHKDDEDKFFPMSGVLIFGKQGAGN |
| | SNVDLDNVMITNEEEIKTINPVATEQYGTVATNLQSANRGDLLLSTAPAT |
| | GTVNSQGALPGMVWQDRDVYLQGPIWAKIPHTDGHFHPSPLMGGFGLKHP |
| | PPQILIKNTPVPANPPTTFSPAKFASFITQYSTGQVSVEIEWELQKENSK |
| | RWNPEIQYTSNYNKSTNVDFAVDINGVYSEPRPIGTRYLTRNL |
| Anc80- | MAADGYLPDWLEDNLSEGIREWWDLKPGAPKPKANQQKQDDGRGLVLPGY |
| 156 | KYLGPFNGLDKGEPVNAADAAALEHDKAYDQQLKAGDNPYLRYNHADAEF |
| SEQ ID | QERLQEDTSFGGNLGRAVFQAKKRVLEPLGLVEEGAKTAPGKKRPVEQSP |
| NO: | QEPDSSSGIGKKGQQPAKKRLNFGQTGDSESVPDPQPLGEPPAAPSGVGS |
| 44889 | NTMAAGGGAPMADNNEGADGVGNASGNWHCDSTWLGDRVITTSTRTWALP |
| | TYNNHLYKQISSQSGASTNDNTYFGYSTPWGYFDFNRFHCHFSPRDWQRL |
| | INNNWGFRPKRLNFKLFNIQVKEVTINDGTTTIANNLTSTVQVFTDSEYQ |
| | LPYVLGSAHQGCLPPFPADVFMIPQYGYLTLNNGSQAVGRSSFYCLEYFP |
| | SQMLRTGNNFQFSYTFEDVPFHSSYAHSQSLDRLMNPLIDQYLYYLSRTQ |
| | TTSGTAGNRTLQFSQAGPSSMANQAKNWLPGPCYRQQRVSKTANQNNNSN |
| | FAWTGATKYHLNGRDSLVNPGPAMATHKDDEDKFFPMSGVLIFGKQGAGN |
| | SNVDLDNVMITSEEEIKTTNPVATEEYGTVATNLQSANTAPATGTVNSQG |
| | ALPGMVWQNRDVYLQGPIWAKIPHTDGHFHPSPLMGGFGLKHPPPQILIK |
| | NTPVPANPPTTFSPAKFASFITQYSTGQVSVEIEWELQKENSKRWNPEIQ |
| | YTSNYNKSTNVDFAVDINGVYSEPRPIGTRYLTRNL |
| Anc80- | MAADGYLPDWLEDNLSEGIREWWDLKPGAPKPKANQQKQDDGRGLVLPGY |
| 156- | KYLGPFNGLDKGEPVNAADAAALEHDKAYDQQLKAGDNPYLRYNHADAEF |
| Decol | QERLQEDTSFGGNLGRAVFQAKKRVLEPLGLVEEGAKTAPGKKRPVEQSP |
| SEQ ID | QEPDSSSGIGKKGQQPAKKRLNFGQTGDSESVPDPQPLGEPPAAPSGVGS |
| NO: | NTMAAGGGAPMADNNEGADGVGNASGNWHCDSTWLGDRVITTSTRTWALP |
| 44890 | TYNNHLYKQISSQSGASTNDNTYFGYSTPWGYFDFNRFHCHFSPRDWQRL |
| | INNNWGFRPKRLNFKLFNIQVKEVTINDGTTTIANNLTSTVQVFTDSEYQ |
| | LPYVLGSAHQGCLPPFPADVFMIPQYGYLTLNNGSQAVGRSSFYCLEYFP |
| | SQMLRTGNNFQFSYTFEDVPFHSSYAHSQSLDRLMNPLIDQYLYYLSRTQ |
| | TTSGTAGNRTLQFSQAGPSSMANQAKNWLPGPCYRQQRVSKTANQNNNSN |
| | FAWTGATKYHLNGRDSLVNPGPAMATHKDDEDKFFPMSGVLIFGKQGAGN |
| | SNVDLDNVMITSEEEIKTTNPVATEEYGTVATNLQSANRGDLLLSTAPAT |
| | GTVNSQGALPGMVWQNRDVYLQGPIWAKIPHTDGHFHPSPLMGGFGLKHP |
| | PPQILIKNTPVPANPPTTFSPAKFASFITQYSTGQVSVEIEWELQKENSK |
| | RWNPEIQYTSNYNKSTNVDFAVDINGVYSEPRPIGTRYLTRNL |
| Anc80- | MAADGYLPDWLEDNLSEGIREWWDLKPGAPKPKANQQKQDDGRGLVLPGY |
| 751- | KYLGPFNGLDKGEPVNAADAAALEHDKAYDQQLKAGDNPYLRYNHADAEF |
| decol | QERLQEDTSFGGNLGRAVFQAKKRVLEPLGLVEEGAKTAPGKKRPVEQSP |
| SEQ ID | QEPDSSSGIGKKGQQPAKKRLNFGQTGDSESVPDPQPLGEPPAAPSGVGS |
| NO: | NTMASGGGAPMADNNEGADGVGNASGNWHCDSTWLGDRVITTSTRTWALP |
| 44891 | TYNNHLYKQISSQSGASTNDNTYFGYSTPWGYFDFNRFHCHFSPRDWQRL |
| | INNNWGFRPKRLNFKLFNIQVKEVTINDGTTTIANNLTSTVQVFTDSEYQ |
| | LPYVLGSAHQGCLPPFPADVFMIPQYGYLTLNNGSQAVGRSSFYCLEYFP |
| | SQMLRTGNNFEFSYTFEDVPFHSSYAHSQSLDRLMNPLIDQYLYYLSRTQ |
| | TTSGTAGNRELQFSQAGPSSMANQAKNWLPGPCYRQQRVSKTTNQNNNSN |
| | FAWTGATKYHLNGRDSLVNPGPAMATHKDDEDKFFPMSGVLIFGKQGAGN |
| | SNVDLDNVMITSEEEIKTTNPVATEEYGTVATNLQSSNRGDLLLSTAPAT |
| | GTVNSQGALPGMVWQDRDVYLQGPIWAKIPHTDGHFHPSPLMGGFGLKHP |
| | PPQILIKNTPVPANPPTTFSPAKFASFITQYSTGQVSVEIEWELQKENSK |
| | RWNPEIQYTSNYNKSTNVDFAVDINGVYSEPRPIGTRYLTRNL |
| Anc80~ | MAADGYLPDWLEDNLSEGIREWWDLKPGAPKPKANQQKQDDGRGLVLPGY |
| 751 | KYLGPFNGLDKGEPVNAADAAALEHDKAYDQQLKAGDNPYLRYNHADAEF |
| SEQ ID | QERLQEDTSFGGNLGRAVFQAKKRVLEPLGLVEEGAKTAPGKKRPVEQSP |
| NO: | QEPDSSSGIGKKGQQPAKKRLNFGQTGDSESVPDPQPLGEPPAAPSGVGS |
| 44916 | NTMASGGGAPMADNNEGADGVGNASGNWHCDSTWLGDRVITTSTRTWALP |
| | TYNNHLYKQISSQSGASTNDNTYFGYSTPWGYFDFNRFHCHFSPRDWQRL |
| | INNNWGFRPKRLNFKLFNIQVKEVTINDGTTTIANNLTSTVQVFTDSEYQ |
| | LPYVLGSAHQGCLPPFPADVFMIPQYGYLTLNNGSQAVGRSSFYCLEYFP |
| | SQMLRTGNNFEFSYTFEDVPFHSSYAHSQSLDRLMNPLIDQYLYYLSRTQ |
| | TTSGTAGNRELQFSQAGPSSMANQAKNWLPGPCYRQQRVSKTTNQNNNSN |
| | FAWTGATKYHLNGRDSLVNPGPAMATHKDDEDKFFPMSGVLIFGKQGAGN |
| | SNVDLDNVMITSEEEIKTTNPVATEEYGTVATNLQSSNTAPATGTVNSQG |
| | ALPGMVWQDRDVYLQGPIWAKIPHTDGHFHPSPLMGGFGLKHPPPQILIK |
| | NTPVPANPPTTFSPAKFASFITQYSTGQVSVEIEWELQKENSKRWNPEIQ |
| | YTSNYNKSTNVDFAVDINGVYSEPRPIGTRYLTRNL |
| Anc80- | MAADGYLPDWLEDNLSEGIREWWDLKPGAPKPKANQQKQDDGRGLVLPGY |
| 1029- | KYLGPFNGLDKGEPVNAADAAALEHDKAYDQQLKAGDNPYLRYNHADAEF |
| decol | QERLQEDTSFGGNLGRAVFQAKKRVLEPLGLVEEGAKTAPGKKRPVEQSP |
| SEQ ID | QEPDSSSGIGKKGQQPARKRLNFGQTGDSESVPDPQPLGEPPAAPSGVGS |
| NO: | NTMAAGGGAPMADNNEGADGVGNASGNWHCDSTWLGDRVITTSTRTWALP |
| 44892 | TYNNHLYKQISSQSGASTNDNTYFGYSTPWGYFDFNRFHCHFSPRDWQRL |
| | INNNWGFRPKKLNFKLFNIQVKEVTINDGTTTIANNLTSTVQVFTDSEYQ |
| | LPYVLGSAHQGCLPPFPADVFMIPQYGYLTLNNGSQAVGRSSFYCLEYFP |
| | SQMLRTGNNFQFSYTFEDVPFHSSYAHSQSLDRLMNPLIDQYLYYLSRTQ |
| | TTSGTAGNRTLQFSQAGPSSMANQAKNWLPGPCYRQQRVSKTTNQNNNSN |
| | FAWTGATKYHLNGRDSLVNPGPAMATHKDDEDKFFPMSGVLIFGKQGAGN |
| | SNVDLDNVMITNEEEIKTTNPVATEEYGTVATNLQSANRGDLLLSTAPAT |
| | GTVNSQGALPGMVWQDRDVYLQGPIWAKIPHTDGHFHPSPLMGGFGLKHP |
| | PPQILIKNTPVPANPPTTFSPAKFASFITQYSTGQVSVEIEWELQKENSK |
| | RWNPEIQYTSNYNKSTNVDFAVDINGVYSEPRPIGTRYLTRNL |
| Anc80- | MAADGYLPDWLEDNLSEGIREWWDLKPGAPKPKANQQKQDDGRGLVLPGY |
| 1029 | KYLGPFNGLDKGEPVNAADAAALEHDKAYDQQLKAGDNPYLRYNHADAEF |
| SEQ ID | QERLQEDTSFGGNLGRAVFQAKKRVLEPLGLVEEGAKTAPGKKRPVEQSP |
| NQ | QEPDSSSGIGKKGQQPARKRLNFGQTGDSESVPDPQPLGEPPAAPSGVGS |
| 44917 | NTMAAGGGAPMADNNEGADGVGNASGNWHCDSTWLGDRVITTSTRTWALP |
| | TYNNHLYKQISSQSGASTNDNTYFGYSTPWGYFDFNRFHCHFSPRDWQRL |
| | INNNWGFRPKKLNFKLFNIQVKEVTTNDGTTTIANNLTSTVQVFTDSEYQ |
| | LPYVLGSAHQGCLPPFPADVFMIPQYGYLTLNNGSQAVGRSSFYCLEYFP |
| | SQMLRTGNNFQFSYTFEDVPFHSSYAHSQSLDRLMNPLIDQYLYYLSRTQ |
| | TTSGTAGNRTLQFSQAGPSSMANQAKNWLPGPCYRQQRVSKTTNQNNNSN |
| | FAWTGATKYHLNGRDSLVNPGPAMATHKDDEDKFFPMSGVLIFGKQGAGN |
| | SNVDLDNVMITNEEEIKTINPVATEEYGTVATNLQSANTAPATGTVNSQG |
| | ALPGMVWQDRDVYLQGPIWAKIPHTDGHFHPSPLMGGFGLKHPPPQILIK |
| | NTPVPANPPTTFSPAKFASFITQYSTGQVSVEIEWELQKENSKRWNPEIQ |
| | YTSNYNKSTNVDFAVDINGVYSEPRPIGTRYLTRNL |
| Anc80- | MAADGYLPDWLEDNLSEGIREWWDLKPGAPKPKANQQKQDDGRGLVLPGY |
| 1712 | KYLGPFNGLDKGEPVNAADAAALEHDKAYDQQLKAGDNPYLRYNHADAEF |
| SEQ ID | QERLQEDTSFGGNLGRAVFQAKKRVLEPLGLVEEGAKTAPGKKRPVEQSP |
| NO: | QEPDSSSGIGKKGQQPARKRLNFGQTGDSESVPDPQPLGEPPAAPSGVGS |
| 44893 | NTMASGGGAPMADNNEGADGVGNASGNWHCDSTWLGDRVITTSTRTWALP |
| | TYNNHLYKQISSQSGASTNDNTYFGYSTPWGYFDFNRFHCHFSPRDWQRL |
| | INNNWGFRPKRLNFKLFNIQVKEVTINDGTTTIANNLTSTVQVFTDSEYQ |
| | LPYVLGSAHQGCLPPFPADVFMIPQYGYLTLNNGSQAVGRSSFYCLEYFP |
| | SQMLRTGNNFQFSYTFEDVPFHSSYAHSQSLDRLMNPLIDQYLYYLSRTQ |
| | TTSGTAGNRELQFSQAGPSSMANQAKNWLPGPCYRQQRVSKTANQNNNSN |
| | FAWTGATKYHLNGRDSLVNPGPAMATHKDDEDKFFPMSGVLIFGKQGAGN |
| | SNVDLDNVMITNEEEIKTTNPVATEQYGTVATNLQSANTAPATGTVNSQG |
| | ALPGMVWQNRDVYLQGPIWAKIPHTDGHFHPSPLMGGFGLKHPPPQILIK |
| | NTPVPANPPTTFSPAKFASFITQYSTGQVSVEIEWELQKENSKRWNPEIQ |
| | YTSNYNKSTNVDFAVDINGVYSEPRPIGTRYLTRNL |
| Anc80- | MAADGYLPDWLEDNLSEGIREWWDLKPGAPKPKANQQKQDDGRGLVLPGY |
| 1712- | KYLGPFNGLDKGEPVNAADAAALEHDKAYDQQLKAGDNPYLRYNHADAEF |
| decol | QERLQEDTSFGGNLGRAVFQAKKRVLEPLGLVEEGAKTAPGKKRPVEQSP |
| SEQ ID | QEPDSSSGIGKKGQQPARKRLNFGQTGDSESVPDPQPLGEPPAAPSGVGS |
| NQ | NTMASGGGAPMADNNEGADGVGNASGNWHCDSTWLGDRVITTSTRTWALP |
| 44894 | TYNNHLYKQISSQSGASTNDNTYFGYSTPWGYFDFNRFHCHFSPRDWQRL |
| | INNNWGFRPKRLNFKLFNIQVKEVTINDGTTTIANNLTSTVQVFTDSEYQ |
| | LPYVLGSAHQGCLPPFPADVFMIPQYGYLTLNNGSQAVGRSSFYCLEYFP |
| | SQMLRTGNNFQFSYTFEDVPFHSSYAHSQSLDRLMNPLIDQYLYYLSRTQ |
| | TTSGTAGNRELQFSQAGPSSMANQAKNWLPGPCYRQQRVSKTANQNNNSN |
| | FAWTGATKYHLNGRDSLVNPGPAMATHKDDEDKFFPMSGVLIFGKQGAGN |
| | SNVDLDNVMITNEEEIKTTNPVATEQYGTVATNLQSANRGDLLLSTAPAT |
| | GTVNSQGALPGMVWQNRDVYLQGPIWAKIPHTDGHFHPSPLMGGFGLKHP |
| | PPQILIKNTPVPANPPTTFSPAKFASFITQYSTGQVSVEIEWELQKENSK |
| | RWNPEIQYTSNYNKSTNVDFAVDINGVYSEPRPIGTRYLTRNL |
| Anc80 | MAADGYLPDWLEDNLSEGIREWWDLKPGAPKPKANQQKQDDGRGLVLPGY |
| L65 | KYLGPFNGLDKGEPVNAADAAALEHDKAYDQQLKAGDNPYLRYNHADAEF |
| SEQ ID | QERLQEDTSFGGNLGRAVFQAKKRVLEPLGLVEEGAKTAPGKKRPVEQSP |
| NO: | QEPDSSSGIGKKGQQPARKRLNFGQTGDSESVPDPQPLGEPPAAPSGVGS |
| 44895 | NTMAAGGGAPMADNNEGADGVGNASGNWHCDSTWLGDRVITTSTRTWALP |
| | TYNNHLYKQISSQSGGSTNDNTYFGYSTPWGYFDFNRFHCHFSPRDWQRL |
| | INNNWGFRPKKLNFKLFNIQVKEVTTNDGTTTIANNLTSTVQVFTDSEYQ |
| | LPYVLGSAHQGCLPPFPADVFMIPQYGYLTLNNGSQAVGRSSFYCLEYFP |
| | SQMLRTGNNFQFSYTFEDVPFHSSYAHSQSLDRLMNPLIDQYLYYLSRTQ |
| | TTSGTAGNRTLQFSQAGPSSMANQAKNWLPGPCYRQQRVSKTTNQNNNSN |
| | FAWTGATKYHLNGRDSLVNPGPAMATHKDDEDKFFPMSGVLIFGKQGAGN |
| | SNVDLDNVMITNEEEIKTINPVATEEYGTVATNLQSANTAPATGTVNSQG |
| | ALPGMVWQDRDVYLQGPIWAKIPHTDGHFHPSPLMGGFGLKHPPPQILIK |
| | NTPVPANPPTTFSPAKFASFITQYSTGQVSVEIEWELQKENSKRWNPEIQ |
| | YTSNYNKSTNVDFAVDINGVYSEPRPIGTRYLTRNL |
| Anc80 | MAADGYLPDWLEDNLSEGIREWWDLKPGAPKPKANQQKQDDGRGLVLPGY |
| L65 | KYLGPFNGLDKGEPVNAADAAALEHDKAYDQQLKAGDNPYLRYNHADAEF |
| liver | QERLQEDTSFGGNLGRAVFQAKKRVLEPLGLVEEGAKTAPGKKRPVEQSP |
| off | QEPDSSSGIGKKGQQPARKRLNFGQTGDSESVPDPQPLGEPPAAPSGVGS |
| SEQ ID | NTMAAGGGAPMADNNEGADGVGNASGNWHCDSTWLGDRVITTSTRTWALP |
| NO: | TYNNHLYKQISSQSGATTNDNTYFGYSTPWGYFDFNRFHCHFSPRDWQRL |
| 44896 | INNNWGFRPKKLNFKLFNIQVKEVTINDGTTTIANNLTSTVQVFTDSEYQ |
| | LPYVLGSAHQGCLPPFPADVFMIPQYGYLTLNNGSQAVGRSSFYCLEYFP |
| | SQMLRTGNNFQFSYTFEDVPFHSSYAHSQSLDRLMNPLIDQYLYYLSRTQ |
| | TTSGTAGNRTLQFSQAGPSSMANQAKNWLPGPCYRQQRVSKTTNQNNNSN |
| | FAWTGATKYHLNGRDSLVNPGPAMATHKDDEDKFFPMSGVLIFGKQGAGN |
| | SNVDLDNVMITNEEEIKTINPVATEEYGTVATNLQSANTAPATGTVNSQG |
| | ALPGMVWQDRDVYLQGPIWAKIPHTDGHFHPSPLMGGFGLKHPPPQILIK |
| | NTPVPANPPTTFSPAKFASFITQYSTGQVSVEIEWELQKENSKRWNPEIQ |
| | YTSNYNKSTNVDFAVDINGVYSEPRPIGTRYLTRNL |
| Anc80 | MAADGYLPDWLEDNLSEGIREWWDLKPGAPKPKANQQKQDDGRGLVLPGY |
| L65 | KYLGPFNGLDKGEPVNAADAAALEHDKAYDQQLKAGDNPYLRYNHADAEF |
| Deco1 | QERLQEDTSFGGNLGRAVFQAKKRVLEPLGLVEEGAKTAPGKKRPVEQSP |
| SEQ ID | QEPDSSSGIGKKGQQPARKRLNFGQTGDSESVPDPQPLGEPPAAPSGVGS |
| NO: | NTMAAGGGAPMADNNEGADGVGNASGNWHCDSTWLGDRVITTSTRTWALP |
| 44897 | TYNNHLYKQISSQSGGSTNDNTYFGYSTPWGYFDFNRFHCHFSPRDWQRL |
| | INNNWGFRPKKLNFKLFNIQVKEVTINDGTTTIANNLTSTVQVFTDSEYQ |
| | LPYVLGSAHQGCLPPFPADVFMIPQYGYLTLNNGSQAVGRSSFYCLEYFP |
| | SQMLRTGNNFQFSYTFEDVPFHSSYAHSQSLDRLMNPLIDQYLYYLSRTQ |
| | TTSGTAGNRTLQFSQAGPSSMANQAKNWLPGPCYRQQRVSKTTNQNNNSN |
| | FAWTGATKYHLNGRDSLVNPGPAMATHKDDEDKFFPMSGVLIFGKQGAGN |
| | SNVDLDNVMITNEEEIKTTNPVATEEYGTVATNLQSANRGDLLLSTAPAT |
| | GTVNSQGALPGMVWQDRDVYLQGPIWAKIPHTDGHFHPSPLMGGFGLKHP |
| | PPQILIKNTPVPANPPTTFSPAKFASFITQYSTGQVSVEIEWELQKENSK |
| | RWNPEIQYTSNYNKSINVDFAVDINGVYSEPRPIGTRYLTRNL |
| Anc80 | MAADGYLPDWLEDNLSEGIREWWDLKPGAPKPKANQQKQDDGRGLVLPGY |
| L65 | KYLGPFNGLDKGEPVNAADAAALEHDKAYDQQLKAGDNPYLRYNHADAEF |
| Liver | QERLQEDTSFGGNLGRAVFQAKKRVLEPLGLVEEGAKTAPGKKRPVEQSP |
| Off | QEPDSSSGIGKKGQQPARKRLNFGQTGDSESVPDPQPLGEPPAAPSGVGS |
| Deco1 | NTMAAGGGAPMADNNEGADGVGNASGNWHCDSTWLGDRVITTSTRTWALP |
| SEQ ID | TYNNHLYKQISSQSGATTNDNTYFGYSTPWGYFDFNRFHCHFSPRDWQRL |
| NO: | INNNWGFRPKKLNFKLFNIQVKEVTTNDGTTTIANNLTSTVQVFTDSEYQ |
| 44898 | LPYVLGSAHQGCLPPFPADVFMIPQYGYLTLNNGSQAVGRSSFYCLEYFP |
| | SQMLRTGNNFQFSYTFEDVPFHSSYAHSQSLDRLMNPLIDQYLYYLSRTQ |
| | TTSGTAGNRTLQFSQAGPSSMANQAKNWLPGPCYRQQRVSKTTNQNNNSN |
| | FAWTGATKYHLNGRDSLVNPGPAMATHKDDEDKFFPMSGVLIFGKQGAGN |
| | SNVDLDNVMITNEEEIKTTNPVATEEYGTVATNLQSANRGDLLLSTAPAT |
| | GTVNSQGALPGMVWQDRDVYLQGPIWAKIPHTDGHFHPSPLMGGFGLKHP |
| | PPQILIKNTPVPANPPTTFSPAKFASFITQYSTGQVSVEIEWELQKENSK |
| | RWNPEIQYTSNYNKSTNVDFAVDINGVYSEPRPIGTRYLTRNL |
| AAV9 | MAADGYLPDWLEDNLSEGIREWWALKPGAPQPKANQQHQDNARGLVLPGY |
| (MUTI- | KYLGPGNGLDKGEPVNAADAAALEHDKAYDQQLKAGDNPYLKYNHADAEF |
| ATLV | QERLKEDTSFGGNLGRAVFQAKKRLLEPLGLVEEAAKTAPGKKRPVEQSP |
| T013X | QEPDSSAGIGKSGAQPAKKRLNFGQTGDTESVPDPQPIGEPPAAPSGVGS |
| X38181) | LTMASGGGAPVADNNEGADGVGSSSGNWHCDSQWLGDRVITTSTRTWALP |
| SEQ ID | TYNNHLYKQISNSTSGASTNDNAYFGYSTPWGYFDFNRFHCHFSPRDWQR |
| NO: | LINNNWGFRPKRLNFKLFNIQVKEVTDNNGVKTIANNLTSTVQVFTDSDY |
| 44900 | QLPYVLGSAHEGCLPPFPADVFMIPQYGYLTLNDGSQAVGRSSFYCLEYF |
| | PSQMLRTGNNFQFSYEFENVPFHSSYAHSQSLDRLMNPLIDQYLYYLSKT |
| | INGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSAQRGDRGQIAQAQT |
| | GWVQNQGILPGMVWQDRDVYLQGPIWAKIPHTDGNFHPSPLMGGFGMKHP |
| | PPQILIKNTPVPADPPTAFNKDKLNSFITQYSTGQVSVEIEWELQKENSK |
| | RWNPEIQYTSNYYKSNNVEFAVNTEGVYSEPRPIGTRYLTRNL |
| AAV9 | MAADGYLPDWLEDNLSEGIREWWALKPGAPQPKANQQHQDNARGLVLPGY |
| (MUTI- | KYLGPGNGLDKGEPVNAADAAALEHDKAYDQQLKAGDNPYLKYNHADAEF |
| ATLV | QERLKEDTSFGGNLGRAVFQAKKRLLEPLGLVEEAAKTAPGKKRPVEQSP |
| T013X | QEPDSSAGIGKSGAQPAKKRLNFGQTGDTESVPDPQPIGEPPAAPSGVGS |
| X40049 | LTMASGGGAPVADNNEGADGVGSSSGNWHCDSQWLGDRVITTSTRTWALP |
| SEQ ID | TYNNHLYKQISNSTSGASTNDNAYFGYSTPWGYFDFNRFHCHFSPRDWQR |
| NO: | LINNNWGFRPKRLNFKLFNIQVKEVTDNNGVKTIANNLTSTVQVFTDSDY |
| 44901 | QLPYVLGSAHEGCLPPFPADVFMIPQYGYLTLNDGSQAVGRSSFYCLEYF |
| | PSQMLRTGNNFQFSYEFENVPFHSSYAHSQSLDRLMNPLIDQYLYYLSKT |
| | INGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEBEIKTTNPVATESYGQVATNHQSAQRGDRSVVAQAQT |
| | GWVQNQGILPGMVWQDRDVYLQGPIWAKIPHTDGNFHPSPLMGGFGMKHP |
| | PPQILIKNTPVPADPPTAFNKDKLNSFITQYSTGQVSVEIEWELQKENSK |
| | RWNPEIQYTSNYYKSNNVEFAVNTEGVYSEPRPIGTRYLTRNL |
| AAV9 | MAADGYLPDWLEDNLSEGIREWWALKPGAPQPKANQQHQDNARGLVLPGY |
| (MUTI- | KYLGPGNGLDKGEPVNAADAAALEHDKAYDQQLKAGDNPYLKYNHADAEF |
| ATLV | QERLKEDTSFGGNLGRAVFQAKKRLLEPLGLVEEAAKTAPGKKRPVEQSP |
| T013X | QEPDSSAGIGKSGAQPAKKRLNFGQTGDTESVPDPQPIGEPPAAPSGVGS |
| X38170 | LTMASGGGAPVADNNEGADGVGSSSGNWHCDSQWLGDRVITTSTRTWALP |
| SEQ ID | TYNNHLYKQISNSTSGASTNDNAYFGYSTPWGYFDFNRFHCHFSPRDWQR |
| NO: | LINNNWGFRPKRLNFKLFNIQVKEVTDNNGVKTIANNLTSTVQVFTDSDY |
| 44902 | QLPYVLGSAHEGCLPPFPADVFMIPQYGYLTLNDGSQAVGRSSFYCLEYF |
| | PSQMLRTGNNFQFSYEFENVPFHSSYAHSQSLDRLMNPLIDQYLYYLSKT |
| | INGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSAQRGDRGNQAQAQT |
| | GWVQNQGILPGMVWQDRDVYLQGPIWAKIPHTDGNFHPSPLMGGFGMKHP |
| | PPQILIKNTPVPADPPTAFNKDKLNSFITQYSTGQVSVEIEWELQKENSK |
| | RWNPEIQYTSNYYKSNNVEFAVNTEGVYSEPRPIGTRYLTRNL |
| AAV9 | MAADGYLPDWLEDNLSEGIREWWALKPGAPQPKANQQHQDNARGLVLPGY |
| (MUTI- | KYLGPGNGLDKGEPVNAADAAALEHDKAYDQQLKAGDNPYLKYNHADAEF |
| ATLV | QERLKEDTSFGGNLGRAVFQAKKRLLEPLGLVEEAAKTAPGKKRPVEQSP |
| T013X | QEPDSSAGIGKSGAQPAKKRLNFGQTGDTESVPDPQPIGEPPAAPSGVGS |
| X39374 | LTMASGGGAPVADNNEGADGVGSSSGNWHCDSQWLGDRVITTSTRTWALP |
| SEQ ID | TYNNHLYKQISNSTSGASTNDNAYEGYSTPWGYFDFNRFHCHFSPRDWQR |
| NO: | LINNNWGFRPKRLNFKLFNIQVKEVTDNNGVKTIANNLTSTVQVFTDSDY |
| 44903 | QLPYVLGSAHEGCLPPFPADVFMIPQYGYLTLNDGSQAVGRSSFYCLEYF |
| | PSQMLRTGNNFQFSYEFENVPFHSSYAHSQSLDRLMNPLIDQYLYYLSKT |
| | INGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSAQRGDRNVVAQAQT |
| | GWVQNQGILPGMVWQDRDVYLQGPIWAKIPHTDGNFHPSPLMGGFGMKHP |
| | PPQILIKNTPVPADPPTAFNKDKLNSFITQYSTGQVSVEIEWELQKENSK |
| | RWNPEIQYTSNYYKSNNVEFAVNTEGVYSEPRPIGTRYLTRNL |
| AAV9 | MAADGYLPDWLEDNLSEGIREWWALKPGAPQPKANQQHQDNARGLVLPGY |
| (MUTI- | KYLGPGNGLDKGEPVNAADAAALEHDKAYDQQLKAGDNPYLKYNHADAEF |
| ATLV | QERLKEDTSFGGNLGRAVFQAKKRLLEPLGLVEEAAKTAPGKKRPVEQSP |
| TQ13X | QEPDSSAGIGKSGAQPAKKRLNFGQTGDTESVPDPQPIGEPPAAPSGVGS |
| X20169 | LTMASGGGAPVADNNEGADGVGSSSGNWHCDSQWLGDRVITTSTRTWALP |
| SEQ ID | TYNNHLYKQISNSTSGASTNDNAYFGYSTPWGYFDFNRFHCHFSPRDWQR |
| NO: | LINNNWGFRPKRLNFKLFNIQVKEVIDNNGVKTIANNLTSTVQVFTDSDY |
| 44904 | QLPYVLGSAHEGCLPPFPADVFMIPQYGYLTLNDGSQAVGRSSFYCLEYF |
| | PSQMLRTGNNFQFSYEFENVPFHSSYAHSQSLDRLMNPLIDQYLYYLSKT |
| | INGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSAQRGDHVNLAQAQT |
| | GWVQNQGILPGMVWQDRDVYLQGPIWAKIPHTDGNFHPSPLMGGFGMKHP |
| | PPQILIKNTPVPADPPTAFNKDKLNSFITQYSTGQVSVEIEWELQKENSK |
| | RWNPEIQYTSNYYKSNNVEFAVNTEGVYSEPRPIGTRYLTRNL |
| AAV9 | MAADGYLPDWLEDNLSEGIREWWALKPGAPQPKANQQHQDNARGLVLPGY |
| (MUTI- | KYLGPGNGLDKGEPVNAADAAALEHDKAYDQQLKAGDNPYLKYNHADAEF |
| ATLV | QERLKEDTSFGGNLGRAVFQAKKRLLEPLGLVEEAAKTAPGKKRPVEQSP |
| T013X | QEPDSSAGIGKSGAQPAKKRLNFGQTGDTESVPDPQPIGEPPAAPSGVGS |
| X49911 | LTMASGGGAPVADNNEGADGVGSSSGNWHCDSQWLGDRVITTSTRTWALP |
| SEQ ID | TYNNHLYKQISNSTSGASTNDNAYFGYSTPWGYFDFNRFHCHFSPRDWQR |
| NO: | LINNNWGFRPKRLNFKLFNIQVKEVTDNNGVKTIANNLTSTVQVFTDSDY |
| 44905 | QLPYVLGSAHEGCLPPFPADVFMIPQYGYLTLNDGSQAVGRSSFYCLEYF |
| | PSQMLRTGNNFQFSYEFENVPFHSSYAHSQSLDRLMNPLIDQYLYYLSKT |
| | INGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSAQRGDVRSIAQAQT |
| | GWVQNQGILPGMVWQDRDVYLQGPIWAKIPHTDGNFHPSPLMGGFGMKH |
| | PPPQILIKNTPVPADPPTAFNKDKLNSFITQYSTGQVSVEIEWELQKEN |
| | SKRWNPEIQYTSNYYKSNNVEFAVNTEGVYSEPRPIGTRYLTRNL |
| AAV9 | MAADGYLPDWLEDNLSEGIREWWALKPGAPQPKANQQHQDNARGLVLPGY |
| (MUTI- | KYLGPGNGLDKGEPVNAADAAALEHDKAYDQQLKAGDNPYLKYNHADAEF |
| ATLV | QERLKEDTSFGGNLGRAVFQAKKRLLEPLGLVEEAAKTAPGKKRPVEQSP |
| T013X | QEPDSSAGIGKSGAQPAKKRLNFGQTGDTESVPDPQPIGEPPAAPSGVGS |
| X38249 | LTMASGGGAPVADNNEGADGVGSSSGNWHCDSQWLGDRVITTSTRTWALP |
| SEQ ID | TYNNHLYKQISNSTSGASTNDNAYFGYSTPWGYFDFNRFHCHFSPRDWQR |
| NO: | LINNNWGFRPKRLNFKLFNIQVKEVTDNNGVKTIANNLTSTVQVFTDSDY |
| 44906 | QLPYVLGSAHEGCLPPFPADVFMIPQYGYLTLNDGSQAVGRSSFYCLEYF |
| | PSQMLRTGNNFQFSYEFENVPFHSSYAHSQSLDRLMNPLIDQYLYYLSKT |
| | INGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSAQRGDRGVVAQAQT |
| | GWVQNQGILPGMVWQDRDVYLQGPIWAKIPHTDGNFHPSPLMGGFGMKHP |
| | PPQILIKNTPVPADPPTAFNKDKLNSFITQYSTGQVSVEIEWELQKENSK |
| | RWNPEIQYTSNYYKSNNVEFAVNTEGVYSEPRPIGTRYLTRNL |
| AAV9 | MAADGYLPDWLEDNLSEGIREWWALKPGAPQPKANQQHQDNARGLVLPGY |
| (MUTI- | KYLGPGNGLDKGEPVNAADAAALEHDKAYDQQLKAGDNPYLKYNHADAEF |
| ATLV | QERLKEDTSFGGNLGRAVFQAKKRLLEPLGLVEEAAKTAPGKKRPVEQSP |
| T013X | QEPDSSAGIGKSGAQPAKKRLNFGQTGDTESVPDPQPIGEPPAAPSGVGS |
| X38219 | LTMASGGGAPVADNNEGADGVGSSSGNWHCDSQWLGDRVITTSTRTWALP |
| SEQ ID | TYNNHLYKQISNSTSGASTNDNAYFGYSTPWGYFDFNRFHCHFSPRDWQR |
| NO: | LINNNWGFRPKRLNFKLFNIQVKEVTDNNGVKTIANNLTSTVQVFTDSDY |
| 44907 | QLPYVLGSAHEGCLPPFPADVFMIPQYGYLTLNDGSQAVGRSSFYCLEYF |
| | PSQMLRTGNNFQFSYEFENVPFHSSYAHSQSLDRLMNPLIDQYLYYLSKT |
| | INGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSAQRGDRGSVAQAQT |
| | GWVQNQGILPGMVWQDRDVYLQGPIWAKIPHTDGNFHPSPLMGGFGMKHP |
| | PPQILIKNTPVPADPPTAFNKDKLNSFITQYSTGQVSVEIEWELQKENSK |
| | RWNPEIQYTSNYYKSNNVEFAVNTEGVYSEPRPIGTRYLTRNL |
| AAV9 | MAADGYLPDWLEDNLSEGIREWWALKPGAPQPKANQQHQDNARGLVLPGY |
| (MUTI- | KYLGPGNGLDKGEPVNAADAAALEHDKAYDQQLKAGDNPYLKYNHADAEF |
| ATLV | QERLKEDTSFGGNLGRAVFQAKKRLLEPLGLVEEAAKTAPGKKRPVEQSP |
| T013X | QEPDSSAGIGKSGAQPAKKRLNFGQTGDTESVPDPQPIGEPPAAPSGVGS |
| X26512 | LTMASGGGAPVADNNEGADGVGSSSGNWHCDSQWLGDRVITTSTRTWALP |
| SEQ ID | TYNNHLYKQISNSTSGASTNDNAYFGYSTPWGYFDFNRFHCHFSPRDWQR |
| NO: | LINNNWGFRPKRLNFKLFNIQVKEVTDNNGVKTIANNLTSTVQVFTDSDY |
| 44908 | QLPYVLGSAHEGCLPPFPADVFMIPQYGYLTLNDGSQAVGRSSFYCLEYF |
| | PSQMLRTGNNFQFSYEFENVPFHSSYAHSQSLDRLMNPLIDQYLYYLSKT |
| | INGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSAQRGDKSSIAQAQT |
| | GWVQNQGILPGMVWQDRDVYLQGPIWAKIPHTDGNFHPSPLMGGFGMKHP |
| | PPQILIKNTPVPADPPTAFNKDKLNSFITQYSTGQVSVEIEWELQKENSK |
| | RWNPEIQYTSNYYKSNNVEFAVNTEGVYSEPRPIGTRYLTRNL |
| AAV9 | MAADGYLPDWLEDNLSEGIREWWALKPGAPQPKANQQHQDNARGLVLPGY |
| (MUTI- | KYLGPGNGLDKGEPVNAADAAALEHDKAYDQQLKAGDNPYLKYNHADAEF |
| ATLV | QERLKEDTSFGGNLGRAVFQAKKRLLEPLGLVEEAAKTAPGKKRPVEQSP |
| T013X | QEPDSSAGIGKSGAQPAKKRLNFGQTGDTESVPDPQPIGEPPAAPSGVGS |
| X3824 | LTMASGGGAPVADNNEGADGVGSSSGNWHCDSQWLGDRVITTSTRTWALP |
| 7 | TYNNHLYKQISNSTSGASTNDNAYFGYSTPWGYFDFNRFHCHFSPRDWQR |
| SEQ ID | LINNNWGFRPKRLNFKLFNIQVKEVTDNNGVKTIANNLTSTVQVFTDSDY |
| NO: | QLPYVLGSAHEGCLPPFPADVFMIPQYGYLTLNDGSQAVGRSSFYCLEYF |
| 44909 | PSQMLRTGNNFQFSYEFENVPFHSSYAHSQSLDRLMNPLIDQYLYYLSKT |
| | INGSGQNQQTLKFSVAGPSNMAVQGRNYIPGPSYRQQRVSTTVTQNNNSE |
| | FAWPGASSWALNGRNSLMNPGPAMASHKEGEDRFFPLSGSLIFGKQGTGR |
| | DNVDADKVMITNEEEIKTTNPVATESYGQVATNHQSAQRGDRGVSAQAQT |
| | GWVQNQGILPGMVWQDRDVYLQGPIWAKIPHTDGNFHPSPLMGGFGMKHP |
| | PPQILIKNTPVPADPPTAFNKDKLNSFITQYSTGQVSVEIEWELQKENSK |
| | RWNPEIQYTSNYYKSNNVEFAVNTEGVYSEPRPIGTRYLTRNL |
| Library | P₁P₂P₅P₄P₅P₆P₇P₈NDNP₁₂ |
| Consensus | |
| SEQ | P₁P₂P₃P₄GGP₇P₈NDNP₁₂ |
| ID NO: | |
| 44921 | |
| (Top | |
| 28 | |
| Consensus) | |
| SEQ | P₁P₂TP₄GGP₇P₈NDNP₁₂ |
| ID NO: | |
| 44922 | |
| (Peptide | |
| segment 1) | |
| SEQ | P₁P₂QP₄GGP₇P₈NDNP₁₂ |
| ID NO: | |
| 44923 | |
| (Peptide | |
| Segment 2) | |
| SEQ | P₁P₂TP₄GGP₇TNDNP₁₂ |
| ID NO: | |
| 44924 | |
| (Peptide | |
| Segment 3) | |
| SEQ | NP₂TP₄GGP₇P₈NDNP₁₂ |
| ID NO: | |
| 44925 | |
| (Peptid? | |
| Segment 4) | |
| SEQ | SP₂TP₄GGP₇P₈NDNP₁₂ |
| ID NO: | |
| 44926 | |
| (Peptide | |
| Segment 5) | |
| SEQ | NSTSGGP₇P₈NDNH |
| ID NO: | |
| 44927 | |
| (group | |
| 1) | |
| SEQ | NSTTGGP₇P₈NDNH |
| ID NO: | |
| 44928 | |
| (group | |
| 2) | |
| SEQ | SGQTGGP₇P₈NDNH |
| ID NO: | |
| 44929 | |
| (group | |
| 3) | |
| SEQ | SGTAGGP₇P₈NDNT |
| ID NO: | |
| 44930 | |
| (group | |
| 4) | |
| SEQ | SGTSGGP₇P₈NDNA |
| ID NO: | |
| 44931 | |
| (group | |
| 5) | |
| SEQ | SGTTGGP₇P₈NDNT |
| ID NO: | |
| 44932 | |
| (group | |
| 6) | |
| SEQ | SSTAGGP₇P₈NDNA |
| ID NO: | |
| 44933 | |
| (group | |
| 7) | |

### 10. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### 11. TABLES

Provided below is Table 27. Abbreviations for Table 27 are as follows: Bicep—Bicep femoris, Diap—Diaphragm, Flex—Flexor Digitorum Profundus, Vent.—Heart VentricleWall Left, Tibia—Tibialis Anterior, and Tricep—Triceps Brachii. Table 27 shows individual tissue enrichment scores for each AAV variant. Data is provided for tissues including Liver, Bicep, Diap., Flex, Vent., Tibia, and Tricep.

Lengthy table referenced here
US20260062450A1-20260305-T00001
Please refer to the end of the specification for access instructions.

Provided below is Table 28. Table 28 shows individual tissue enrichment scores for each AAV variant. Data is provided for tissues including Liver DNA, Liver RNA, Heart DNA, and Heart RNA.

| | SEQ | | | | | | | SEQ | | | | | |
|---|---|---|---|---|---|---|---|---|---|---|---|---|---|
| M to | ID | | Liver | Liver | Heart | Heart | M to | ID | | Liver | Liver | Heart | Heart |
| variant_id | NO: | Sequence | DNA | RNA | DNA | RNA | variant_id | NO: | Sequence | DNA | RNA | DNA | RNA |
| ATLVT012XX0001 | 44935 | NGAAAAASNDNA | −0.1 | −1.02 | −3.29 | −1.09 | ATLVT012XX0047 | 44981 | NGAAGGSTNDNH | −1.48 | −2 | −0.83 | 0.43 |
| ATLVT012XX0002 | 44936 | NGAAAAASNDNH | −1.1 | −1.11 | −1.48 | −1.39 | ATLVT012XX0048 | 44982 | NGAAGGSTNDNT | 1.18 | 0.18 | 0.18 | 0.07 |
| ATLVT012XX0003 | 44937 | NGAAAAASNDNT | 0 | −1.4 | −2.39 | 0.39 | ATLVT012XX0049 | 44983 | NGASAAASNDNA | −0.75 | −0.72 | −0.12 | −0.36 |
| ATLVT012XX0004 | 44938 | NGAAAAATNDNA | 0.61 | −1.74 | −1.27 | 0.05 | ATLVT012XX0050 | 44984 | NGASAAASNDNH | −1.51 | −0.47 | 0.94 | 0.13 |
| ATLVT012XX0005 | 44939 | NGAAAAATNDNH | 0.22 | −1.43 | −1.17 | 0.13 | ATLVT012XX0051 | 44985 | NGASAAASNDNT | −0.8 | −2.03 | 1.52 | 1.52 |
| ATLVT012XX0006 | 44940 | NGAAAAATNDNT | 0.28 | −0.25 | 0.13 | 0.81 | ATLVT012XX0052 | 44986 | NGASAAATNDNA | −0.91 | −2.41 | −0.43 | −0.46 |
| ATLVT012XX0007 | 44941 | NGAAAASSNDNA | −0.94 | −1.69 | −1.95 | −0.21 | ATLVT012XX0053 | 44987 | NGASAAATNDNH | 0.76 | −1.46 | −0.77 | 2.1 |
| ATLVT012XX0008 | 44942 | NGAAAASSNDNH | −0.58 | −0.74 | −0.8 | 0.55 | ATLVT012XX0054 | 44988 | NGASAAATNDNT | 0.51 | −2.42 | 1.16 | −0.8 |
| ATLVT012XX0009 | 44943 | NGAAAASSNDNT | −1.46 | −1.08 | 0.44 | −0.11 | ATLVT012XX0055 | 44989 | NGASAASSNDNA | −1.86 | −0.46 | 0.95 | −0.11 |
| ATLVT012XX0010 | 44944 | NGAAAASTNDNA | −1.75 | −1.09 | −1.93 | −0.85 | ATLVT012XX0056 | 44990 | NGASAASSNDNH | −0.37 | −2.25 | 0.32 | −0.37 |
| ATLVT012XX0011 | 44945 | NGAAAASTNDNH | −2.33 | −1.99 | −1.46 | −0.75 | ATLVT012XX0057 | 44991 | NGASAASSNDNT | −1.03 | −1.97 | 1.1 | −1.41 |
| ATLVT012XX0012 | 44946 | NGAAAASTNDNT | −0.43 | −1.16 | −0.73 | 0.4 | ATLVT012XX0058 | 44992 | NGASAASTNDNA | −1.8 | −2.3 | 0.16 | −2.63 |
| ATLVT012XX0013 | 44947 | NGAAAGASNDNA | −2.61 | −1.1 | −2.25 | −1.47 | ATLVT012XX0059 | 44993 | NGASAASTNDNH | −1.36 | −1.94 | 0.95 | −1.93 |
| ATLVT012XX0014 | 44948 | NGAAAGASNDNH | 0.11 | −0.98 | −2.33 | −2.09 | ATLVT012XX0060 | 44994 | NGASAASTNDNT | 0.06 | −2.2 | 0.11 | −0.07 |
| ATLVT012XX0015 | 44949 | NGAAAGASNDNT | −0.9 | −0.47 | −2.99 | −2.66 | ATLVT012XX0061 | 44995 | NGASAGASNDNA | −3.61 | 1.79 | 0.21 | 0.82 |
| ATLVT012XX0016 | 44950 | NGAAAGATNDNA | −1.6 | 0.92 | −2.92 | −1.71 | ATLVT012XX0062 | 44996 | NGASAGASNDNH | −3.21 | 1.82 | 0.26 | 0.19 |
| ATLVT012XX0017 | 44951 | NGAAAGATNDNH | −1.24 | 2.7 | −1.91 | −0.66 | ATLVT012XX0063 | 44997 | NGASAGASNDNT | −3.4 | −0.02 | −0.74 | −0.1 |
| ATLVT012XX0018 | 44952 | NGAAAGATNQNT | −2.1 | −0.09 | −1.96 | −2.6 | ATLVT012XX0064 | 44998 | NGASAGATNDNA | −2.86 | −0.06 | 1.15 | 1.5 |
| ATLVT012XX0019 | 44953 | NGAAAGSSNDNA | −1.44 | 2.03 | −2.93 | −1.69 | ATLVT012XX0065 | 44999 | NGASAGATNDNH | −1.48 | −0.37 | 0.29 | 2.01 |
| ATLVT012XX0020 | 44954 | NGAAAGSSNDNH | −2.95 | 2.18 | −2.77 | −3.07 | ATLVT012XX0066 | 45000 | NGASAGATNDNT | −2.44 | 0.06 | 0.52 | 0.71 |
| ATLVT012XX0021 | 44955 | NGAAAGSSNDNT | −0.75 | 1.05 | −1.65 | −3.3 | ATLVT012XX0067 | 45001 | NGASAGSSNDNA | −0.66 | 3.12 | 0.58 | 0.28 |
| ATLVT012XX0022 | 44956 | NGAAAGSTNDNA | 0.01 | 1.63 | −1.29 | −1.96 | ATLVT012XX0068 | 45002 | NGASAGSSNDNH | −1.88 | 0.57 | −0.34 | −0.62 |
| ATLVT012XX0023 | 44957 | NGAAAGSTNDNH | −0.71 | 1.18 | −0.69 | −1.71 | ATLVT012XX0069 | 45003 | NGASAGSSNDNT | −0.88 | 1.03 | −0.64 | −1.29 |
| ATLVT012XX0024 | 44958 | NGAAAGSTNDNT | −0.03 | 2.88 | −2.11 | −3.25 | ATLVT012XX0070 | 45004 | NGASAGSTNDNA | 0.04 | 3.74 | 0.52 | −0.83 |
| ATLVT012XX0025 | 44959 | NGAAGAASNDNA | −3.92 | −2.55 | −2.31 | −2.71 | ATLVT012XX0071 | 45005 | NGASAGSTNDNH | 0.37 | 1.44 | −0.64 | 0.83 |
| ATLVT012XX0026 | 44960 | NGAAGAASNDNH | −3.48 | −4.91 | −3.94 | −4.24 | ATLVT012XX0072 | 45006 | NGASAGSTNDNT | 0.81 | 3.5 | −0.36 | −1.06 |
| ATLVT012XX0027 | 44961 | NGAAGAASNDNT | −3.69 | −3.25 | −2.48 | −1.12 | ATLVT012XX0073 | 45007 | NGASGAASNDNA | −3.79 | −0.63 | −3.52 | −2.2 |
| ATLVT012XX0028 | 44962 | NGAAGAATNDNA | −1.62 | −2.26 | −2.16 | −1.13 | ATLVT012XX0074 | 45008 | NGASGAASNDNH | −2.76 | 2 | −2.83 | −2.21 |
| ATLVT012XX0029 | 44963 | NGAAGAATNDNH | −2.47 | −0.28 | −2.57 | −0.93 | ATLVT012XX0075 | 45009 | NGASGAASNDNT | −2.95 | −0.79 | −2.28 | −0.38 |
| ATLVT012XX0030 | 44964 | NGAAGAATNDNT | −3.53 | | −2.25 | −1.87 | ATLVT012XX0076 | 45010 | NGASGAATNDNA | −3.81 | −1.35 | −1.98 | −1.88 |
| ATLVT012XX0031 | 44965 | NGAAGASSNDNA | −3.72 | −2.86 | −1.4 | −0.96 | ATLVT012XX0077 | 45011 | NGASGAATNDNH | −2.47 | 0.17 | 0.03 | −1.65 |
| ATLVT012XX0032 | 44966 | NGAAGASSNDNH | −3.2 | −5.77 | −1.64 | −1.58 | ATLVT012XX0078 | 45012 | NGASGAATNDNT | −2.26 | 0.35 | −0.53 | −1.59 |
| ATLVT012XX0033 | 44967 | NGAAGASSNDNT | −1.91 | −3.48 | −1.27 | −1.6 | ATLVT012XX0079 | 45013 | NGASGASSNDNA | −1.57 | 0.36 | 1.27 | −2.28 |
| ATLVT012XX0034 | 44968 | NGAAGASTNDNA | −4.11 | −2.09 | −0.53 | −0.48 | ATLVT012XX0080 | 45014 | NGASGASSNDNH | 0.56 | −1.22 | −0.08 | −1.68 |
| ATLVT012XX0035 | 44969 | NGAAGASTNDNH | −2.13 | −0.74 | −1.87 | −0.73 | ATLVT012XX0081 | 45015 | NGASGASSNDNT | −0.19 | 0.07 | 0.81 | −2.09 |
| ATLVT012XX0036 | 44970 | NGAAGASTNDNT | −3.67 | −1.18 | −1.59 | −1.07 | ATLVT012XX0082 | 45016 | NGASGASTNDNA | −1.81 | 0.99 | 0.92 | 0.52 |
| ATLVT012XX0037 | 44971 | NGAAGGASNDNA | −1.79 | −0.61 | −3.31 | −2.02 | ATLVT012XX0083 | 45017 | NGASGASTNDNH | −0.69 | −0.55 | −0.51 | 1.17 |
| ATLVT012XX0038 | 44972 | NGAAGGASNDNH | 1.41 | 1.09 | 1.58 | 0.79 | ATLVT012XX0084 | 45018 | NGASGASTNDNT | 0.29 | −1.18 | 0.05 | 1.22 |
| ATLVT012XX0039 | 44973 | NGAAGGASNDNT | −0.43 | −1.28 | −4.61 | −0.48 | ATLVT012XX0085 | 45019 | NGASGGASNDNA | 1.73 | 0.15 | 0.99 | −0.28 |
| ATLVT012XX0040 | 44974 | NGAAGGATNDNA | 0.46 | −0.27 | −3.4 | −0.17 | ATLVT012XX0086 | 45020 | NGASGGASNDNH | 3.11 | 0.51 | 0.13 | 0.49 |
| ATLVT012XX0041 | 44975 | NGAAGGATNDNH | 0.89 | 1.07 | −2.35 | −0.71 | ATLVT012XX0087 | 45021 | NGASGGASNDNT | 2.42 | 2.32 | 0.37 | −0.02 |
| ATLVT012XX0042 | 44976 | NGAAGGATNQNT | 0.42 | 0.73 | −2.37 | −0.85 | ATLVT012XX0088 | 45022 | NGASGGATNDNA | −1.68 | −0.92 | 0.96 | −0.91 |
| ATLVT012XX0043 | 44977 | NGAAGGSSNDNA | −0.1 | 0.39 | −1.02 | −1.21 | ATLVT012XX0089 | 45023 | NGASGGATNHNH | 1.16 | 2.28 | 0.17 | 1.48 |
| ATLVT012XX0044 | 44978 | NGAAGGSSNDNH | 0.26 | −0.03 | −0.86 | −0.07 | ATLVT012XX0090 | 45024 | NGASGGATNDNT | 0.67 | 0.64 | −0.01 | 0.68 |
| ATLVT012XX0045 | 44979 | NGAAGGSSNDNT | −0.22 | 0.08 | −2.54 | −2.83 | ATLVT012XX0091 | 45025 | NGASGGSSNDNA | 1.06 | 2.53 | 0.27 | 0.03 |
| ATLVT012XX0046 | 44980 | NGAAGGSTNDNA | −1.7 | −0.79 | −1.43 | −1.89 | ATLVT012XX0092 | 45026 | NGASGGSSNDNH | 0.69 | −0.19 | 0.27 | 1.73 |
| ATLVT012XX0093 | 45027 | NGASGGSSNDNT | 2.75 | 0.28 | 0.62 | 0.9 | ATLVT012XX0139 | 45073 | NGATGGSSNDNA | 1.95 | 2.75 | 0.95 | 1.39 |
| ATLVT012XX0094 | 45028 | NGASGGSTNDNA | 2.28 | 2.78 | −0.11 | 1.49 | ATLVT012XX0140 | 45074 | NGATGGSSNDNH | −0.04 | 2.52 | 0.38 | 1.42 |
| ATLVT012XX0095 | 45029 | NGASGGSTNDNH | 3.01 | 1.56 | 0 | 2.37 | ATLVT012XX0141 | 45075 | NGATGGSSNDNT | 1.86 | 3 | 0.42 | −0.47 |
| ATLVT012XX0096 | 45030 | NGASGGSTNDNT | 3.12 | 1.98 | 0.42 | 0.45 | ATLVT012XX0142 | 45076 | NGATGGSTNDNA | 0.21 | 1.92 | 0.82 | 0.14 |
| ATLVT012XX0097 | 45031 | NGATAAASNDNA | 0.46 | −2.76 | −0.53 | 1.02 | ATLVT012XX0143 | 45077 | NGATGGSTNDNH | −0.77 | 2.52 | 0.66 | 2.48 |
| ATLVT012XX0098 | 45032 | NGATAAASNDNH | 3.53 | −1.94 | −0.41 | 1.85 | ATLVT012XX0144 | 45078 | NGATGGSTNDNT | 2.64 | 3.37 | 0.42 | 0.47 |
| ATLVT012XX0099 | 45033 | NGATAAASNDNT | 1.4 | −1.72 | 0.61 | −0.12 | ATLVT012XX0145 | 45079 | NGEAAAASNDNA | −2.54 | −1.83 | 0.61 | 1.44 |
| ATLVT012XX0100 | 45034 | NGATAAATNDNA | −0.48 | −1.03 | −0.78 | 1.75 | ATLVT012XX0146 | 45080 | NGEAAAASNDNH | −2.97 | −1.3 | 0.14 | −1.87 |
| ATLVT012XX0101 | 45035 | NGATAAATNDNH | 0.26 | 0.41 | −0.15 | 3.19 | ATLVT012XX0147 | 45081 | NGEAAAASNDNT | −1.6 | −0.36 | 0.87 | −1.55 |
| ATLVT012XX0102 | 45036 | NGATAAATNDNT | 0.15 | −2.17 | 0.81 | 1.4 | ATLVT012XX0148 | 45082 | NGEAAAATNDNA | −0.06 | 0.26 | −0.38 | −0.1 |
| ATLVT012XX0103 | 45037 | NGATAASSNDNA | −1.79 | 2.07 | −0.76 | −1.86 | ATLVT012XX0149 | 45083 | NGEAAAATNDNH | 1.97 | 0.06 | 1.34 | 0.99 |
| ATLVT012XX0104 | 45038 | NGATAASSNDNH | 0.75 | −1.24 | −0.03 | −0.85 | ATLVT012XX0150 | 45084 | NGEAAAATNDNT | 1.58 | 0.49 | 1.16 | 0.33 |
| ATLVT012XX0105 | 45039 | NGATAASSNDNT | 0.03 | −1.39 | 1.14 | 0.39 | ATLVT012XX0151 | 45085 | NGEAAASSNDNA | 2.32 | 1.53 | 0.32 | −1.73 |
| ATLVT012XX0106 | 45040 | NGATAASTNDNA | 1.56 | −1.08 | 0.26 | −0.36 | ATLVT012XX0152 | 45086 | NGEAAASSNDNH | 1.68 | −0.62 | −0.92 | −2 |
| ATLVT012XX0107 | 45041 | NGATAASTNDNH | −0.04 | −0.31 | 0.11 | −0.08 | ATLVT012XX0153 | 45087 | NGEAAASSNDNT | 4.3 | 0.78 | 0.32 | 0.46 |
| ATLVT012XX0108 | 45042 | NGATAASTNDNT | 2.48 | 1.03 | 0.51 | −1.27 | ATLVT012XX0154 | 45088 | NGEAAASTNDNA | 0.14 | −0.3 | 0.86 | −1.7 |
| ATLVT012XX0109 | 45043 | NGATAGASNDNA | −1.53 | 1.64 | −0.09 | −2.13 | ATLVT012XX0155 | 45089 | NGEAAASTNDNH | −2.4 | −0.7 | −0.09 | −0.77 |
| ATLVT012XX0110 | 45044 | NGATAGASNDNH | 0.09 | −0.16 | −0.73 | −0.15 | ATLVT012XX0156 | 45090 | NGEAAASTNDNT | −1.03 | 3.38 | −0.53 | −0.15 |
| ATLVT012XX0111 | 45045 | NGATAGASNDNT | 2.26 | −0.74 | −0.66 | −0.89 | ATLVT012XX0157 | 45091 | NGEAAGASNDNA | −0.76 | 2.1 | −1.78 | −2.65 |
| ATLVT012XX0112 | 45046 | NGATAGATNDNA | 0.62 | −0.18 | −0.71 | 0.33 | ATLVT012XX0158 | 45092 | NGEAAGASNDNH | −0.22 | −2.02 | −1.21 | −2.43 |
| ATLVT012XX0113 | 45047 | NGATAGATNDNH | 3.31 | 1.97 | 1.21 | 3.92 | ATLVT012XX0159 | 45093 | NGEAAGASNDNT | 2.28 | −1.35 | −2.28 | −3.86 |
| ATLVT012XX0114 | 45048 | NGATAGATNDNT | 2.05 | 1.47 | 0.82 | 1.44 | ATLVT012XX0160 | 45094 | NGEAAGATNDNA | −0.43 | −2.02 | −1.63 | −0.46 |
| ATLVT012XX0115 | 45049 | NGATAGSSNDNA | −0.4 | 1.06 | 0.53 | −1.67 | ATLVT012XX0161 | 45095 | NGEAAGATNDNH | 0.1 | −1.97 | −0.68 | 2.56 |
| ATLVT012XX0116 | 45050 | NGATAGSSNDNH | −1.11 | −1.36 | 0.31 | 1.44 | ATLVT012XX0162 | 45096 | NGEAAGATNDNT | −1.06 | −0.81 | −1.49 | 0.32 |
| ATLVT012XX0117 | 45051 | NGATAGSSNDNT | 0.27 | −0.9 | −0.31 | −0.37 | ATLVT012XX0163 | 45097 | NGEAAGSSNDNA | −1.6 | −2.12 | 0.3 | −2.79 |
| ATLVT012XX0118 | 45052 | NGATAGSTNDNA | 1.5 | −1.66 | 0.64 | 1.22 | ATLVT012XX0164 | 45098 | NGEAAGSSNDNH | −1.09 | −1.54 | 0.43 | −1.68 |
| ATLVT012XX0119 | 45053 | NGATAGSTNDNH | 2.39 | −0.57 | 1.06 | 1.38 | ATLVT012XX0165 | 45099 | NGEAAGSSNDNT | −1.44 | −2.62 | 0.56 | −4.31 |
| ATLVT012XX0120 | 45054 | NGATAGSTNDNT | 0.02 | −1.16 | 1.27 | 0.93 | ATLVT012XX0166 | 45100 | NGEAAGSTNDNA | 0.21 | −1.87 | 0.61 | −2.79 |
| ATLVT012XX0121 | 45055 | NGATGAASNDNA | −1.82 | −1.75 | −1.06 | 1.48 | ATLVT012XX0167 | 45101 | NGEAAGSTNDNH | −0.08 | −2.76 | 0.98 | −0.53 |
| ATLVT012XX0122 | 45056 | NGATGAASNDNH | −0.97 | −0.4 | −0.76 | 0.72 | ATLVT012XX0168 | 45102 | NGEAAGSTNDNT | −1.07 | −2.86 | 0.3 | −1.08 |
| ATLVT012XX0123 | 45057 | NGATGAASNDNT | −0.71 | −0.19 | −1.83 | 1.99 | ATLVT012XX0169 | 45103 | NGEAGAASNDNA | −2.65 | −0.59 | −0.82 | −3.35 |
| ATLVT012XX0124 | 45058 | NGATGAATNDNA | −0.88 | 1.98 | 1.14 | −1.54 | ATLVT012XX0170 | 45104 | NGEAGAASNDNH | −2.31 | −1.83 | 0.79 | −1.61 |
| ATLVT012XX0125 | 45059 | NGATGAATNDNH | −1.96 | 0.66 | 0.3 | 1.43 | ATLVT012XX0171 | 45105 | NGEAGAASNDNT | −3.99 | −2.13 | 0.52 | −2.29 |
| ATLVT012XX0126 | 45060 | NGATGAATNDNT | −1.34 | 2.47 | 0.83 | −1.51 | ATLVT012XX0172 | 45106 | NGEAGAATNDNA | −4.22 | 1.73 | −0.33 | −0.62 |
| ATLVT012XX0127 | 45061 | NGATGASSNDNA | 0.45 | 1.43 | −0.43 | −0.65 | ATLVT012XX0173 | 45107 | NGEAGAATNDNH | −3.39 | 0.11 | 0.68 | 1.31 |
| ATLVT012XX0128 | 45062 | NGATGASSNDNH | −0.76 | −0.82 | 0.54 | 0.81 | ATLVT012XX0174 | 45108 | NGEAGAATNDNT | −3.9 | 1.21 | −0.38 | −0.76 |
| ATLVT012XX0129 | 45063 | NGATGASSNDNT | 0.07 | 0.99 | −0.12 | 0.36 | ATLVT012XX0175 | 45109 | NGEAGASSNDNA | −2.69 | 0.28 | −0.26 | −0.09 |
| ATLVT012XX0130 | 45064 | NGATGASTNDNA | −0.92 | 2.39 | 1.47 | 0.64 | ATLVT012XX0176 | 45110 | NGEAGASSNDNH | −1.5 | 1.19 | 0.4 | −1.49 |
| ATLVT012XX0131 | 45065 | NGATGASTNDNH | 0.23 | 3.01 | 1.2 | 2.25 | ATLVT012XX0177 | 45111 | NGEAGASSNDNT | −3.09 | 0.96 | −0.17 | 1.53 |
| ATLVT012XX0132 | 45066 | NGATGASTNDNT | −1.15 | 0.31 | 1.72 | 1.24 | ATLVT012XX0178 | 45112 | NGEAGASTNDNA | 1.11 | 2.6 | 0.15 | 0.12 |
| ATLVT012XX0133 | 45067 | NGATGGASNDNA | −1.35 | 0.1 | −0.63 | 2.01 | ATLVT012XX0179 | 45113 | NGEAGASTNDNH | −1.17 | 0.55 | −0.01 | −0.06 |
| ATLVT012XX0134 | 45068 | NGATGGASNDNH | −0.99 | 1.2 | 1.2 | 1.09 | ATLVT012XX0180 | 45114 | NGEAGASTNDNT | 1.38 | −0.49 | 0.48 | −0.87 |
| ATLVT012XX0135 | 45069 | NGATGGASNDNT | 0.78 | 2.22 | 0.97 | 1.08 | ATLVT012XX0181 | 45115 | NGEAGGASNDNA | −0.74 | 0.46 | −0.75 | −2.2 |
| ATLVT012XX0136 | 45070 | NGATGGATNDNA | 0.24 | 1.98 | 0.2 | 0.52 | ATLVT012XX0182 | 45116 | NGEAGGASNDNH | −0.99 | 4.12 | 0.72 | 1.22 |
| ATLVT012XX0137 | 45071 | NGATGGATNDNH | 0.56 | 7.03 | 0.23 | 2.69 | ATLVT012XX0183 | 45117 | NGEAGGASNDNT | −0.82 | 0.91 | −1.11 | −1.95 |
| ATLVT012XX0138 | 45072 | NGATGGATNDNT | 0.17 | 6.78 | 0.28 | 0.39 | ATLVT012XX0184 | 45118 | NGEAGGATNDNA | −1.8 | 0.87 | −0.64 | −1.32 |
| ATLVT012XX0185 | 45119 | NGEAGGATNDNH | 0.75 | 3.1 | 1.09 | 1.38 | ATLVT012XX0231 | 45165 | NGESGGASNDNT | 2.46 | −0.02 | 0.11 | −0.62 |
| ATLVT012XX0186 | 45120 | NGEAGGATNDNT | −1.4 | 0.52 | 0.52 | −1.75 | ATLVT012XX0232 | 45166 | NGESGGATNDNA | −1.14 | 0.51 | 0.23 | −0.59 |
| ATLVT012XX0187 | 45121 | NGEAGGSSNDNA | −0.59 | 0.33 | −0.63 | 1.17 | ATLVT012XX0233 | 45167 | NGESGGATNDNH | 0.67 | 1.52 | 0.59 | 3.2 |
| ATLVT012XX0188 | 45122 | NGEAGGSSNDNH | 1.2 | −1.68 | 0.11 | 1.43 | ATLVT012XX0234 | 45168 | NGESGGATNDNT | −1.63 | 0.56 | 1.18 | 1.28 |

| | | | | | | | | | | | | | |
|---|---|---|---|---|---|---|---|---|---|---|---|---|---|
| ATLVT012XX0189 | 45123 | NGEAGGSSNDNT | −0.57 | 0.95 | 0.16 | −0.91 | ATLVT012XX0235 | 45169 | NGESGGSSNDNA | −0.2 | −0.34 | 0.03 | 1.5 |
| ATLVT012XX0190 | 45124 | NGEAGGSTNDNA | 0.59 | 2.2 | −0.46 | 0.63 | ATLVT012XX0236 | 45170 | NGESGGSSNDNH | −0.26 | 0.07 | −0.13 | 0.73 |
| ATLVT012XX0191 | 45125 | NGEAGGSTNDNH | −1.23 | 1.72 | 0.52 | 0.88 | ATLVT012XX0237 | 45171 | NGESGGSSNDNT | 3.23 | 1.17 | 0.73 | 1.24 |
| ATLVT012XX0192 | 45126 | NGEAGGSTNDNT | −0.74 | 3.89 | 0.47 | 2.14 | ATLVT012XX0238 | 45172 | NGESGGSTNDNA | 2.16 | 2.22 | 0.69 | 1.19 |
| ATLVT012XX0193 | 45127 | NGESAAASNDNA | −1.37 | −1.15 | 1.2 | 2.67 | ATLVT012XX0239 | 45173 | NGESGGSTNDNH | 3.41 | 0.43 | 0.47 | 1.18 |
| ATLVT012XX0194 | 45128 | NGESAAASNDNH | −0.42 | 2.12 | 0.15 | 1.08 | ATLVT012XX0240 | 45174 | NGESGGSTNDNT | 2.06 | 3.76 | 1.05 | 0.2 |
| ATLVT012XX0195 | 45129 | NGESAAASNDNT | 0.45 | −0.5 | 0.57 | 0.71 | ATLVT012XX0241 | 45175 | NGETAAASNDNA | −1.35 | −0.12 | −0.38 | −0.03 |
| ATLVT012XX0196 | 45130 | NGESAAATNDNA | 0.26 | 3.34 | 0.45 | 0.95 | ATLVT012XX0242 | 45176 | NGETAAASNDNH | −2.18 | 0.85 | −0.46 | 0.04 |
| ATLVT012XX0197 | 45131 | NGESAAATNDNH | −1.29 | 0.65 | 0.63 | 0.17 | ATLVT012XX0243 | 45177 | NGETAAASNDNT | −0.93 | 1.42 | 0 | −0.15 |
| ATLVT012XX0198 | 45132 | NGESAAATNDNT | −1.14 | 3.25 | 0.52 | 0.69 | ATLVT012XX0244 | 45178 | NGETAAATNDNA | −3.55 | −0.49 | 0.92 | −1.22 |
| ATLVT012XX0199 | 45133 | NGESAASSNDNA | −1.23 | 1.37 | 0.43 | −0.74 | ATLVT012XX0245 | 45179 | NGETAAATNDNH | −2.03 | 3.53 | 0.56 | −0.26 |
| ATLVT012XX0200 | 45134 | NGESAASSNDNH | −1.02 | −1.16 | 0.02 | −1.17 | ATLVT012XX0246 | 45180 | NGETAAATNDNT | −0.87 | −0.23 | 0.53 | 0.31 |
| ATLVT012XX0201 | 45135 | NGESAASSNDNT | −1.71 | −1.48 | −0.01 | −0.09 | ATLVT012XX0247 | 45181 | NGETAASSNDNA | 0.02 | −1.15 | −0.34 | −1.26 |
| ATLVT012XX0202 | 45136 | NGESAASTNDNA | −1.5 | −0.48 | 0.11 | 0.54 | ATLVT012XX0248 | 45182 | NGETAASSNDNH | −0.13 | 1.43 | 0.36 | −0.59 |
| ATLVT012XX0203 | 45137 | NGESAASTNDNH | 1.22 | −0.05 | 0.6 | −0.55 | ATLVT012XX0249 | 45183 | NGETAASSNDNT | 0.54 | −1.59 | −0.49 | −0.27 |
| ATLVT012XX0204 | 45138 | NGESAASTNDNT | 0.4 | 0.47 | 0.05 | 1.09 | ATLVT012XX0250 | 45184 | NGETAASTNDNA | 0.19 | −1.24 | 0.26 | 0.32 |
| ATLVT012XX0205 | 45139 | NGESAGASNDNA | −1.56 | −0.62 | −0.11 | −1.63 | ATLVT012XX0251 | 45185 | NGETAASTNDNH | −2.21 | 0.61 | −0.66 | −0.4 |
| ATLVT012XX0206 | 45140 | NGESAGASNDNH | −0.93 | 1.14 | −0.67 | −1.72 | ATLVT012XX0252 | 45186 | NGETAASTNDNT | −0.87 | −1.87 | 0.7 | 0.74 |
| ATLVT012XX0207 | 45141 | NGESAGASNDNT | −1.75 | −0.54 | −0.29 | −0.3 | ATLVT012XX0253 | 45187 | NGETAGASNDNA | 0.11 | −1.44 | 1.22 | −0.72 |
| ATLVT012XX0208 | 45142 | NGESAGATNDNA | −1.37 | −0.67 | −0.22 | −1.59 | ATLVT012XX0254 | 45188 | NGETAGASNDNH | −1.5 | 1.12 | −0.81 | 1.1 |
| ATLVT012XX0209 | 45143 | NGESAGATNDNH | −0.94 | 2.26 | 1.44 | 0.35 | ATLVT012XX0255 | 45189 | NGETAGASNDNT | −1.67 | −1.3 | 0.23 | −1.65 |
| ATLVT012XX0210 | 45144 | NGESAGATNDNT | −1.21 | −0.66 | −0.62 | −0.46 | ATLVT012XX0256 | 45190 | NGETAGATNDNA | −2.59 | −0.6 | 0.65 | −0.57 |
| ATLVT012XX0211 | 45145 | NGESAGSSNDNA | −0.16 | 1.87 | 0.35 | −0.09 | ATLVT012XX0257 | 45191 | NGETAGATNDNH | −2.26 | −0.26 | 0.72 | 1.34 |
| ATLVT012XX0212 | 45146 | NGESAGSSNDNH | −2.71 | −1 | −0.1 | 0.77 | ATLVT012XX0258 | 45192 | NGETAGATNDNT | −2 | 0.21 | 1.54 | −0.5 |
| ATLVT012XX0213 | 45147 | NGESAGSSNDNT | −0.05 | 0.61 | 0.75 | 1.42 | ATLVT012XX0259 | 45193 | NGETAGSSNDNA | −0.48 | −0.51 | 0.4 | 0.49 |
| ATLVT012XX0214 | 45148 | NGESAGSTNDNA | −1.45 | 0.08 | 0.02 | −0.36 | ATLVT012XX0260 | 45194 | NGETAGSSNDNH | −1.76 | −1.86 | −0.5 | −0.39 |
| ATLVT012XX0215 | 45149 | NGESAGSTNDNH | 0.09 | −1.2 | −0.21 | 0.13 | ATLVT012XX0261 | 45195 | NGETAGSSNDNT | 1.61 | −0.44 | −0.53 | −0.99 |
| ATLVT012XX0216 | 45150 | NGESAGSTNDNT | −0.04 | −0.33 | 0.6 | 0.37 | ATLVT012XX0262 | 45196 | NGETAGSTNDNA | −2.18 | −0.37 | 1.1 | −0.74 |
| ATLVT012XX0217 | 45151 | NGESGAASNDNA | −3.17 | 0.01 | 0.31 | −1.78 | ATLVT012XX0263 | 45197 | NGETAGSTNDNH | −1.89 | −1.97 | 0.18 | 0.99 |
| ATLVT012XX0218 | 45152 | NGESGAASNDNH | −2.39 | 1.26 | 0.44 | −2.58 | ATLVT012XX0264 | 45198 | NGETAGSTNDNT | −1.33 | −1.53 | −0.05 | −1.6 |
| ATLVT012XX0219 | 45153 | NGESGAASNDNT | −2.04 | −1.73 | −0.53 | −1.94 | ATLVT012XX0265 | 45199 | NGETGAASNDNA | −2.57 | −2 | 0.23 | −3.17 |
| ATLVT012XX0220 | 45154 | NGESGAATNDNA | −2.9 | −1.42 | −1.25 | −0.2 | ATLVT012XX0266 | 45200 | NGETGAASNDNH | −2.64 | −0.51 | 0.54 | −3.78 |
| ATLVT012XX0221 | 45155 | NGESGAATNDNH | −0.71 | −1.54 | −0.52 | 0.59 | ATLVT012XX0267 | 45201 | NGETGAASNDNT | −0.34 | −1.58 | −0.26 | −3.94 |
| ATLVT012XX0222 | 45156 | NGESGAATNDNT | −3.33 | −1.39 | −1.16 | −0.95 | ATLVT012XX0268 | 45202 | NGETGAATNDNA | −3.69 | −0.91 | −0.69 | −1.89 |
| ATLVT012XX0223 | 45157 | NGESGASSNDNA | −1.36 | −0.9 | 0.66 | −2.83 | ATLVT012XX0269 | 45203 | NGETGAATNDNH | −2.46 | 2.15 | 0.85 | 0.21 |
| ATLVT012XX0224 | 45158 | NGESGASSNDNH | −1.34 | −2.2 | 0.23 | 1.43 | ATLVT012XX0270 | 45204 | NGETGAATNDNT | −1.76 | 0.1 | 1.15 | −0.67 |
| ATLVT012XX0225 | 45159 | NGESGASSNDNT | −1.08 | −2.74 | 0.5 | −0.76 | ATLVT012XX0271 | 45205 | NGETGASSNDNA | −0.61 | −0.61 | 0.05 | 0.28 |
| ATLVT012XX0226 | 45160 | NGESGASTNDNA | 1.76 | 0.38 | 0.94 | −1.02 | ATLVT012XX0272 | 45206 | NGETGASSNDNH | −2.12 | −0.71 | 0.43 | 0.15 |
| ATLVT012XX0227 | 45161 | NGESGASTNDNH | 1.77 | −0.58 | 0.06 | −0.37 | ATLVT012XX0273 | 45207 | NGETGASSNDNT | −1.94 | 2.93 | −0.25 | −1.16 |
| ATLVT012XX0228 | 45162 | NGESGASTNDNT | 3.84 | 1.97 | 0.57 | −0.96 | ATLVT012XX0274 | 45208 | NGETGASTNDNA | 0.4 | −0.08 | 0.27 | −0.68 |
| ATLVT012XX0229 | 45163 | NGESGGASNDNA | 0.25 | 1.73 | 0.13 | −0.4 | ATLVT012XX0275 | 45209 | NGETGASTNDNH | −0.68 | −1.01 | 0.35 | 0.49 |
| ATLVT012XX0230 | 45164 | NGESGGASNDNH | −0.57 | −1.06 | −0.29 | −0.82 | ATLVT012XX0276 | 45210 | NGETGASTNDNT | −0.77 | −0.83 | 0.99 | 0.6 |
| ATLVT012XX0277 | 45211 | NGETGGASNDNA | −1.26 | −1.73 | −0.1 | 0.69 | ATLVT012XX0323 | 45257 | NGQAGASTNDNH | −2.47 | 1.31 | 0.51 | −0.58 |
| ATLVT012XX0278 | 45212 | NGETGGASNDNH | 3.99 | −1.16 | 0.25 | 0.86 | ATLVT012XX0324 | 45258 | NGQAGASTNDNT | −0.69 | −0.03 | 0.78 | −1.62 |
| ATLVT012XX0279 | 45213 | NGETGGASNDNT | 1.18 | −1.31 | 0.27 | −1 | ATLVT012XX0325 | 45259 | NGQAGGASNDNA | −1.9 | −1.02 | −0.65 | −1.46 |
| ATLVT012XX0280 | 45214 | NGETGGATNDNA | 3.31 | 1.18 | 0.88 | 0.15 | ATLVT012XX0326 | 45260 | NGQAGGASNDNH | 1.62 | 3.29 | −1.62 | −0.08 |
| ATLVT012XX0281 | 45215 | NGETGGATNDNH | 2.76 | 1.85 | 0.05 | 2.69 | ATLVT012XX0327 | 45261 | NGQAGGASNDNT | −1.02 | −1.02 | −0.83 | 0.78 |
| ATLVT012XX0282 | 45216 | NGETGGATNDNT | 1.55 | −0.87 | 0.34 | −0.2 | ATLVT012XX0328 | 45262 | NGQAGGATNDNA | 2.42 | 0.14 | 1.34 | 0.2 |
| ATLVT012XX0283 | 45217 | NGETGGSSNDNA | 0.85 | 2.52 | 0.28 | 0.68 | ATLVT012XX0329 | 45263 | NGQAGGATNDNH | 4.11 | 2.02 | 0.97 | 2.13 |
| ATLVT012XX0284 | 45218 | NGETGGSSNDNH | 1.46 | −0.23 | 0.19 | 0.08 | ATLVT012XX0330 | 45264 | NGQAGGATNDNT | 1.49 | −1.12 | 0.35 | 0.36 |
| ATLVT012XX0285 | 45219 | NGETGGSSNDNT | 2.4 | 1.69 | 0.51 | 1.54 | ATLVT012XX0331 | 45265 | NGQAGGSSNDNA | 0.49 | 2.27 | −0.64 | 2.04 |
| ATLVT012XX0286 | 45220 | NGETGGSTNDNA | 2.01 | 2.62 | 0.17 | 0.9 | ATLVT012XX0332 | 45266 | NGQAGGSSNDNH | −1.41 | −0.74 | 0.27 | 1.74 |
| ATLVT012XX0287 | 45221 | NGETGGSTNDNH | 1.56 | −0.7 | 0.33 | 1.15 | ATLVT012XX0333 | 45267 | NGQAGGSSNDNT | 1.48 | −0.55 | −0.33 | 2.82 |
| ATLVT012XX0288 | 45222 | NGETGGSTNDNT | 1.84 | 1.12 | 0.67 | 0.65 | ATLVT012XX0334 | 45268 | NGQAGGSTNDNA | 0.1 | 1.68 | −0.77 | 2 |
| ATLVT012XX0289 | 45223 | NGQAAAASNDNA | −2.94 | −1.33 | −0.48 | 0.53 | ATLVT012XX0335 | 45269 | NGQAGGSTNDNH | 4.14 | 0.08 | −0.34 | 2.79 |
| ATLVT012XX0290 | 45224 | NGQAAAASNDNH | −2.06 | −2.29 | 0.25 | −1.22 | ATLVT012XX0336 | 45270 | NGQAGGSTNDNT | 0.91 | −0.09 | 0.57 | 2.71 |
| ATLVT012XX0291 | 45225 | NGQAAAASNDNT | −2.11 | −2.29 | 0.8 | 0.84 | ATLVT012XX0337 | 45271 | NGQSAAASNDNA | 0.27 | 0.41 | −0.89 | −1.59 |
| ATLVT012XX0292 | 45226 | NGQAAAATNDNA | −3.46 | 2.05 | −0.23 | 0.18 | ATLVT012XX0338 | 45272 | NGQSAAASNDNH | 0.32 | −1.28 | 0.21 | 0.1 |
| ATLVT012XX0293 | 45227 | NGQAAAATNDNH | −1.49 | 0.36 | 0.77 | 2.3 | ATLVT012XX0339 | 45273 | NGQSAAASNDNT | −0.37 | −2.7 | 0.31 | 0.87 |
| ATLVT012XX0294 | 45228 | NGQAAAATNDNT | −3.04 | 3.24 | 0.63 | 0.29 | ATLVT012XX0340 | 45274 | NGQSAAATNDNA | 0.32 | −0.84 | 1.53 | −0.2 |
| ATLVT012XX0295 | 45229 | NGQAAASSNDNA | 1.6 | −0.59 | 0.9 | 1.04 | ATLVT012XX0341 | 45275 | NGQSAAATNDNH | −0.06 | −0.36 | −0.01 | 1.07 |
| ATLVT012XX0296 | 45230 | NGQAAASSNDNH | 0.07 | 1.01 | −0.89 | −1.17 | ATLVT012XX0342 | 45276 | NGQSAAATNDNT | −0.25 | 0.6 | 0.76 | 2 |
| ATLVT012XX0297 | 45231 | NGQAAASSNDNT | 0.89 | 1.17 | −0.54 | −0.36 | ATLVT012XX0343 | 45277 | NGQSAASSNDNA | −0.32 | −1.54 | 0.22 | −0.52 |
| ATLVT012XX0298 | 45232 | NGQAAASTNDNA | 0.74 | −1.33 | −0.11 | −1.72 | ATLVT012XX0344 | 45278 | NGQSAASSNDNH | −1.06 | −2.57 | 0.01 | −0.9 |
| ATLVT012XX0299 | 45233 | NGQAAASTNDNH | 3.33 | −2.03 | −0.67 | 1.79 | ATLVT012XX0345 | 45279 | NGQSAASSNDNT | −1.82 | −2.7 | 0.25 | 0.42 |
| ATLVT012XX0300 | 45234 | NGQAAASTNDNT | 1.24 | −1.08 | −0.47 | 0.28 | ATLVT012XX0346 | 45280 | NGQSAASTNDNA | −0.11 | 1.69 | −0.25 | 0.34 |
| ATLVT012XX0301 | 45235 | NGQAAGASNDNA | 0.37 | −0.39 | −0.65 | −2.92 | ATLVT012XX0347 | 45281 | NGQSAASTNDNH | −1.26 | 0.16 | 0.27 | −1.72 |
| ATLVT012XX0302 | 45236 | NGQAAGASNDNH | −0.49 | 0.57 | −1.37 | −0.92 | ATLVT012XX0348 | 45282 | NGQSAASTNDNT | −2.28 | −0.07 | −0.71 | 0.12 |
| ATLVT012XX0303 | 45237 | NGQAAGASNDNT | −0.85 | 0.44 | −0.2 | −2.01 | ATLVT012XX0349 | 45283 | NGQSAGASNDNA | −2.11 | −2.59 | −0.02 | −0.83 |
| ATLVT012XX0304 | 45238 | NGQAAGATNDNA | −3.15 | 0.05 | −0.76 | −0.85 | ATLVT012XX0350 | 45284 | NGQSAGASNDNH | −1.08 | −2.42 | −1.14 | 1.85 |
| ATLVT012XX0305 | 45239 | NGQAAGATNDNH | −1.37 | 3.39 | −1.26 | 0.44 | ATLVT012XX0351 | 45285 | NGQSAGASNDNT | −1.5 | −2.23 | −0.71 | 1.01 |
| ATLVT012XX0306 | 45240 | NGQAAGATNDNT | −3.19 | 0.12 | −1.43 | −1.56 | ATLVT012XX0352 | 45286 | NGQSAGATNDNA | −1.65 | −2.87 | −0.73 | 0.19 |
| ATLVT012XX0307 | 45241 | NGQAAGSSNDNA | −1.35 | −0.85 | −3.03 | −0.24 | ATLVT012XX0353 | 45287 | NGQSAGATNDNH | −0.41 | −0.97 | −0.85 | 2.07 |
| ATLVT012XX0308 | 45242 | NGQAAGSSNDNH | −0.78 | −1.53 | −0.15 | 0.72 | ATLVT012XX0354 | 45288 | NGQSAGATNDNT | −0.78 | −1.3 | −1.3 | 0.43 |
| ATLVT012XX0309 | 45243 | NGQAAGSSNDNT | −1.18 | −1.23 | −1.14 | −1.27 | ATLVT012XX0355 | 45289 | NGQSAGSSNDNA | 2.1 | −1.86 | −0.67 | 1 |
| ATLVT012XX0310 | 45244 | NGQAAGSTNDNA | −3.18 | −1.83 | −1.54 | 0.16 | ATLVT012XX0356 | 45290 | NGQSAGSSNDNH | 1.35 | 1.16 | −0.35 | 0.79 |
| ATLVT012XX0311 | 45245 | NGQAAGSTNDNH | −0.59 | −0.65 | −1.03 | −1.33 | ATLVT012XX0357 | 45291 | NGQSAGSSNDNT | 1.79 | −0.47 | −0.13 | 0.28 |
| ATLVT012XX0312 | 45246 | NGQAAGSTNDNT | −1.46 | −0.97 | −1.34 | 0.19 | ATLVT012XX0358 | 45292 | NGQSAGSTNDNA | 0.15 | −1.11 | −0.84 | 1.15 |
| ATLVT012XX0313 | 45247 | NGQAGAASNDNA | −2.54 | −2.56 | 0 | 1.14 | ATLVT012XX0359 | 45293 | NGQSAGSTNDNH | 1.48 | −2.25 | −0.7 | 1.94 |
| ATLVT012XX0314 | 45248 | NGQAGAASNDNH | −1.54 | −2.97 | 0.39 | −0.52 | ATLVT012XX0360 | 45294 | NGQSAGSTNDNT | 0.15 | −2.53 | 0.74 | 0.34 |
| ATLVT012XX0315 | 45249 | NGQAGAASNDNT | −1.19 | −1.95 | 0.26 | 2.23 | ATLVT012XX0361 | 45295 | NGQSGAASNDNA | −2.1 | 0.45 | −0.23 | −1.78 |
| ATLVT012XX0316 | 45250 | NGQAGAATNDNA | −0.79 | −1.4 | −2.8 | −0.67 | ATLVT012XX0362 | 45296 | NGQSGAASNDNH | −1.98 | −0.84 | −1.22 | −1.75 |
| ATLVT012XX0317 | 45251 | NGQAGAATNDNH | −1.41 | −0.91 | −0.48 | 0.65 | ATLVT012XX0363 | 45297 | NGQSGAASNDNT | −0.67 | −1.46 | −0.8 | −2.11 |
| ATLVT012XX0318 | 45252 | NGQAGAATNDNT | −0.91 | 0.89 | −1.44 | 1.44 | ATLVT012XX0364 | 45298 | NGQSGAATNDNA | −2.18 | 1.96 | 0.7 | −2.05 |
| ATLVT012XX0319 | 45253 | NGQAGASSNDNA | −0.94 | −1.35 | 0.91 | 1.06 | ATLVT012XX0365 | 45299 | NGQSGAATNDNH | −1.35 | 0.17 | −0.42 | 0.87 |
| ATLVT012XX0320 | 45254 | NGQAGASSNDNH | −1.28 | −0.47 | 0.88 | −0.6 | ATLVT012XX0366 | 45300 | NGQSGAATNDNT | −0.88 | −0.85 | 0.49 | −1.08 |
| ATLVT012XX0321 | 45255 | NGQAGASSNDNT | −0.03 | 0.86 | 0.86 | 1.87 | ATLVT012XX0367 | 45301 | NGQSGASSNDNA | 1.71 | 1.16 | 0.25 | 2 |
| ATLVT012XX0322 | 45256 | NGQAGASTNDNA | −0.86 | −1.88 | 0.76 | −0.84 | ATLVT012XX0368 | 45302 | NGQSGASSNDNH | 2.49 | 3.47 | 0.97 | 0.5 |
| ATLVT012XX0369 | 45303 | NGQSGASSNDNT | 1.08 | −0.87 | 0.65 | −0.41 | ATLVT012XX0415 | 45349 | NGQTGASSNDNA | 2.11 | 0.71 | 0.49 | 0.02 |
| ATLVT012XX0370 | 45304 | NGQSGASTNDNA | −1.17 | 1.82 | 0.52 | −1.06 | ATLVT012XX0416 | 45350 | NGQTGASSNDNH | 1.53 | 0.37 | 0.97 | 1.16 |
| ATLVT012XX0371 | 45305 | NGQSGASTNDNH | −0.11 | −0.44 | 1.5 | 0.06 | ATLVT012XX0417 | 45351 | NGQTGASSNDNT | −0.04 | 0.3 | −0.05 | 0.45 |
| ATLVT012XX0372 | 45306 | NGQSGASTNDNT | 0.93 | −0.5 | 0.63 | 0.48 | ATLVT012XX0418 | 45352 | NGQTGASTNDNA | −1.22 | −0.31 | 0.66 | 0.16 |
| ATLVT012XX0373 | 45307 | NGQSGGASNDNA | 2.5 | 1.97 | 0.32 | 1.4 | ATLVT012XX0419 | 45353 | NGQTGASTNDNH | 1.42 | 0.04 | 0.84 | 1.91 |
| ATLVT012XX0374 | 45308 | NGQSGGASNDNH | 1.06 | 1.21 | 0.1 | 1.53 | ATLVT012XX0420 | 45354 | NGQTGASTNDNT | 0.47 | −1.27 | 0.01 | 2.02 |
| ATLVT012XX0375 | 45309 | NGQSGGASNDNT | 0.48 | 1.46 | 0.26 | 1.71 | ATLVT012XX0421 | 45355 | NGQTGGASNDNA | 1.66 | 0.35 | 0.39 | 2.45 |
| ATLVT012XX0376 | 45310 | NGQSGGATNDNA | 1.12 | −0.49 | 0.72 | 2.26 | ATLVT012XX0422 | 45356 | NGQTGGASNDNH | 1.78 | 0.51 | 1.09 | 3.38 |
| ATLVT012XX0377 | 45311 | NGQSGGATNDNH | 3.41 | 3.25 | 1.88 | 1.98 | ATLVT012XX0423 | 45357 | NGQTGGASNDNT | 0.5 | 1.01 | 0.7 | 1.98 |
| ATLVT012XX0378 | 45312 | NGQSGGATNDNT | 0.96 | 1.7 | 0.83 | 0.98 | ATLVT012XX0424 | 45358 | NGQTGGATNDNA | 1.22 | 2.2 | 0.97 | 1.91 |
| ATLVT012XX0379 | 45313 | NGQSGGSSNDNA | 1.48 | 0.5 | 1.4 | 1.24 | ATLVT012XX0425 | 45359 | NGQTGGATNDNH | 4.07 | 4.83 | 1.62 | 3.8 |
| ATLVT012XX0380 | 45314 | NGQSGGSSNDNH | 0.87 | −0.54 | 0.53 | 2.13 | ATLVT012XX0426 | 45360 | NGQTGGATNDNT | 1.54 | 1.99 | 1.09 | 1.03 |
| ATLVT012XX0381 | 45315 | NGQSGGSSNDNT | 0.54 | 2.84 | 0.34 | 1.79 | ATLVT012XX0427 | 45361 | NGQTGGSSNDNA | 1.24 | 1.35 | 1.2 | 0 |
| ATLVT012XX0382 | 45316 | NGQSGGSTNDNA | 0.96 | 2.68 | 0.48 | 2.74 | ATLVT012XX0428 | 45362 | NGQTGGSSNDNH | −0.27 | −0.27 | 0.38 | 2.07 |
| ATLVT012XX0383 | 45317 | NGQSGGSTNDNH | 3.33 | 2.37 | 0.47 | 1.36 | ATLVT012XX0429 | 45363 | NGQTGGSSNDNT | 2.44 | 2.05 | 1.46 | 1.79 |
| ATLVT012XX0384 | 45318 | NGQSGGSTNDNT | 2.35 | 3.14 | 0.71 | 2.79 | ATLVT012XX0430 | 45364 | NGQTGGSTNDNA | 0.9 | 1.8 | 0.58 | 1.34 |
| ATLVT012XX0385 | 45319 | NGQTAAASNDNA | 1.28 | 0.23 | 1.3 | 1.63 | ATLVT012XX0431 | 45365 | NGQTGGSTNDNH | 0.55 | 3.32 | 0.29 | 2.81 |
| ATLVT012XX0386 | 45320 | NGQTAAASNDNH | 0.9 | 0.74 | 0.82 | 1.84 | ATLVT012XX0432 | 45366 | NGQTGGSTNDNT | 1.8 | 3.01 | −0.02 | 2.71 |
| ATLVT012XX0387 | 45321 | NGQTAAASNDNT | −0.89 | −0.76 | 0.86 | 1.05 | ATLVT012XX0433 | 45367 | NGTAAAASNDNA | 0.86 | 2.43 | −0.08 | −0.19 |
| ATLVT012XX0388 | 45322 | NGQTAAATNDNA | 0.88 | 1.65 | 0.82 | −0.24 | ATLVT012XX0434 | 45368 | NGTAAAASNDNH | −0.11 | −0.62 | −0.17 | 1.77 |
| ATLVT012XX0389 | 45323 | NGQTAAATNDNH | 1.16 | 1.36 | 0.81 | 0.46 | ATLVT012XX0435 | 45369 | NGTAAAASNDNT | 1.18 | 0.46 | −0.9 | −0.82 |
| ATLVT012XX0390 | 45324 | NGQTAAATNDNT | 1.58 | 0.61 | 0.18 | −1.1 | ATLVT012XX0436 | 45370 | NGTAAAATNDNA | −0.13 | 1.62 | 0.3 | −0.07 |
| ATLVT012XX0391 | 45325 | NGQTAASSNDNA | 0.25 | 0.41 | −0.01 | 1.05 | ATLVT012XX0437 | 45371 | NGTAAAATNDNH | 1.93 | 4.61 | 0.92 | 1.22 |
| ATLVT012XX0392 | 45326 | NGQTAASSNDNH | 2.19 | −0.77 | 0.15 | −0.3 | ATLVT012XX0438 | 45372 | NGTAAAATNDNT | 2.36 | 1.14 | 0.62 | 1.94 |
| ATLVT012XX0393 | 45327 | NGQTAASSNDNT | −0.53 | −0.62 | 1.54 | −2.17 | ATLVT012XX0439 | 45373 | NGTAAASSNDNA | 2.73 | 1 | 0.06 | 0.97 |
| ATLVT012XX0394 | 45328 | NGQTAASTNDNA | 1.38 | 4.24 | −0.45 | 2.43 | ATLVT012XX0440 | 45374 | NGTAAASSNDNH | 0.61 | 1.22 | −0.13 | −0.53 |
| ATLVT012XX0395 | 45329 | NGQTAASTNDNH | 1.58 | 4.48 | 1.29 | −0.11 | ATLVT012XX0441 | 45375 | NGTAAASSNDNT | 0.55 | 1.26 | 1.01 | 0.11 |
| ATLVT012XX0396 | 45330 | NGQTAASTNDNT | 2.35 | 1.01 | 1.06 | 0.31 | ATLVT012XX0442 | 45376 | NGTAAASTNDNA | −1.6 | 0.62 | −0.65 | −0.3 |
| ATLVT012XX0397 | 45331 | NGQTAGASNDNA | 1.47 | 2.48 | 0.31 | 0.84 | ATLVT012XX0443 | 45377 | NGTAAASTNDNH | −1.42 | 1.21 | −1.18 | −0.49 |
| ATLVT012XX0398 | 45332 | NGQTAGASNDNH | −1.88 | 0.93 | 0.23 | −0.16 | ATLVT012XX0444 | 45378 | NGTAAASTNDNT | −1.55 | 2.18 | −0.56 | 1.35 |
| ATLVT012XX0399 | 45333 | NGQTAGASNDNT | −1.51 | 2.45 | −1.16 | 1.27 | ATLVT012XX0445 | 45379 | NGTAAGASNDNA | 0.34 | 1.96 | −2.12 | −0.12 |
| ATLVT012XX0400 | 45334 | NGQTAGATNDNA | −0.85 | 0.13 | 0.08 | 2.19 | ATLVT012XX0446 | 45380 | NGTAAGASNDNH | 0 | 3 | −1.51 | 0.51 |
| ATLVT012XX0401 | 45335 | NGQTAGATNDNH | 0.71 | 1.53 | 0.58 | 1.2 | ATLVT012XX0447 | 45381 | NGTAAGASNDNT | 2.36 | 1.26 | −1.78 | −0.88 |
| ATLVT012XX0402 | 45336 | NGQTAGATNDNT | −0.57 | 1.59 | 0.16 | 1.03 | ATLVT012XX0448 | 45382 | NGTAAGATNDNA | 0.26 | −0.21 | −0.97 | 1.39 |
| ATLVT012XX0403 | 45337 | NGQTAGSSNDNA | 2.05 | 1.53 | 0.2 | −0.23 | ATLVT012XX0449 | 45383 | NGTAAGATNDNH | 1.3 | 2.08 | 0.19 | 1.53 |
| ATLVT012XX0404 | 45338 | NGQTAGSSNDNH | −1.53 | 2.02 | 1.02 | 1.55 | ATLVT012XX0450 | 43384 | NGTAAGATNDNT | 0.33 | 0.13 | −0.86 | 0.91 |
| ATLVT012XX0405 | 45339 | NGQTAGSSNDNT | 0.04 | 2.45 | −0.73 | 0.34 | ATLVT012XX0451 | 45385 | NGTAAGSSNDNA | 1.87 | −0.88 | −0.27 | −1.48 |
| ATLVT012XX0406 | 45340 | NGQTAGSTNDNA | 1.5 | 0.83 | 0.67 | 0.91 | ATTVT012XX0452 | 45386 | NGTAAGSSNDNH | −1.09 | −1.9 | −1.02 | 0.84 |
| ATLVT012XX0407 | 45341 | NGQTAGSTNDNH | 1.33 | 2.07 | −0.29 | −0.04 | ATLVT012XX0453 | 45387 | NGTAAGSSNDNT | 0.56 | 1.54 | −0.49 | 0.93 |
| ATLVT012XX0408 | 45342 | NGQTAGSTNDNT | 2.66 | 0.96 | 1.05 | −0.52 | ATLVT012XX0454 | 45388 | NGTAAGSTNDNA | 0.28 | 0.1 | −0.12 | 1.85 |
| ATLVT012XX0409 | 45343 | NGQTGAASNDNA | −0.8 | −1.47 | −1.28 | −0.8 | ATLVT012XX0455 | 45389 | NGTAAGSTNDNE | 2.02 | 1.98 | −1.41 | −0.01 |
| ATLVT012XX0410 | 45344 | NGQTGAASNDNH | −0.98 | −0.51 | −0.02 | 1.57 | ATLVT012XX0456 | 45390 | NGTAAGSTNDNT | 0.33 | 0.45 | −1.97 | 0.92 |
| ATLVT012XX0411 | 45345 | NGQTGAASNDNT | 2.48 | 2.46 | 0.48 | 0.59 | ATLVT012XX0457 | 45391 | NGTAGAASNDNA | 0.21 | −1.43 | −1.99 | −0.33 |
| ATLVT012XX0412 | 45346 | NGQTGAATNDNA | 0.66 | −0.2 | −0.06 | −0.73 | ATLVT012XX0458 | 45392 | NGTAGAASNDNH | 1.43 | 0.19 | −1.7 | −0.91 |
| ATLVT012XX0413 | 45347 | NGQTGAATNDNH | 3.16 | −1.4 | 0.08 | 1.83 | ATLVT012XX0459 | 45393 | NGTAGAASNDNT | 2.17 | −1.99 | −2.6 | −1.69 |
| ATLVT012XX0414 | 45348 | NGQTGAATNDNT | 1.15 | −0.16 | −0.32 | 0.78 | ATLVT012XX0460 | 45394 | NGTAGAATNDNA | 0.53 | −0.76 | −0.38 | −1.68 |
| ATLVT012XX0461 | 45395 | NGTAGAATNDNH | −0.91 | −0.43 | 1.64 | −0.32 | ATLVT012XX0507 | 45441 | NGTSGAASNDNT | −1.57 | −2.33 | 0.4 | −0.24 |
| ATLVT012XX0462 | 45396 | NGTAGAATNDNT | −0.18 | 0.42 | 0.49 | −1.14 | ATLVT012XX0508 | 45442 | NGTSGAATNDNA | −0.66 | 0.21 | −0.5 | 0.42 |
| ATLVT012XX0463 | 45397 | NGTAGASSNDNA | −0.4 | 2.25 | 0.01 | 1.43 | ATLVT012XX0509 | 45443 | NGTSGAATNDNH | 0.2 | 2.13 | 0.95 | 1.8 |
| ATLVT012XX0464 | 45398 | NGTAGASSNDNH | 0.2 | 0.07 | −0.11 | 0.73 | ATLVT012XX0510 | 45444 | NGTSGAATNDNT | 0.68 | 1.28 | −0.2 | −0.06 |
| ATLVT012XX0465 | 45399 | NGTAGASSNDNT | 1.42 | 0.49 | −0.25 | 0.8 | ATLVT012XX0511 | 45445 | NGTSGASSNDNA | 2.34 | 1.7 | 0.41 | 1.21 |
| ATLVT012XX0466 | 45400 | NGTAGASTNDNA | −0.05 | −0.24 | −0.5 | 3.43 | ATLVT012XX0512 | 45446 | NGTSGASSNDNH | 1.92 | −0.13 | 0.68 | 0.57 |
| ATLVT012XX0467 | 45401 | NGTAGASTNDNH | 0.05 | 0.47 | −1.05 | 2.51 | ATLVT012XX0513 | 45447 | NGTSGASSNDNT | 1.81 | 0.73 | 0.02 | −0.22 |
| ATLVT012XX0468 | 45402 | NGTAGASTNDNT | 0.85 | −0.37 | −0.25 | 1.93 | ATLVT012XX0514 | 45448 | NGTSGASTNDNA | 0.83 | 1.65 | 0.9 | 1.8 |
| ATLVT012XX0469 | 45403 | NGTAGGASNDNA | 4.28 | 4.8 | 0.76 | 2.12 | ATLVT012XX0515 | 45449 | NGTSGASTNDNH | 2.17 | 1 | 0.63 | 1.56 |
| ATLVT012XX0470 | 45404 | NGTAGGASNDNH | 3.45 | 4.99 | 0.46 | 3.6 | ATLVT012XX0516 | 45450 | NGTSGASTNDNT | 2.6 | 2.79 | 1.11 | 1.94 |
| ATLVT012XX0471 | 45405 | NGTAGGASNDNT | 5.37 | 7.19 | 1.25 | 1.82 | ATLVT012XX0517 | 45451 | NGTSGGASNDNA | 3.95 | 3.64 | 1.21 | 3.84 |
| ATLVT012XX0472 | 45406 | NGTAGGATNDNA | 1.76 | 1.99 | −0.68 | 1.79 | ATLVT012XX0518 | 45452 | NGTSGGASNDNH | 4.44 | 5.62 | 0.3 | 2.91 |
| ATLVT012XX0473 | 45407 | NGTAGGATNDNH | 4.54 | 3.92 | 1.14 | 2.75 | ATLVT012XX0519 | 45453 | NGTSGGASNDNT | 6.57 | 7.36 | 1.74 | 4.46 |
| ATLVT012XX0474 | 45408 | NGTAGGATNDNT | 4.67 | 6.16 | −1.6 | 1.88 | ATLVT012XX0520 | 45454 | NGTSGGATNDNA | 3.87 | 3.84 | 0.7 | 3.46 |
| ATLVT012XX0475 | 45409 | NGTAGGSSNDNA | 6.36 | 9.13 | 1.27 | 4.03 | ATLVT012XX0521 | 45455 | NGTSGGATNDNH | 5.52 | 5.35 | 1.58 | 4.14 |
| ATLVT012XX0476 | 45410 | NGTAGGSSNDNH | 5.12 | 3.94 | 0.34 | 3.2 | ATLVT012XX0522 | 45456 | NGTSGGATNDNT | 5.43 | 7.85 | 0.93 | 3.84 |
| ATLVT012XX0477 | 45411 | NGTAGGSSNDNT | 9.18 | 10.72 | 2.23 | 4.69 | ATLVT012XX0523 | 45457 | NGTSGGSSNDNA | 6.62 | 9.58 | 1.28 | 4.6 |
| ATLVT012XX0478 | 45412 | NGTAGGSTNDNA | 6.6 | 8.64 | 0.08 | 5.06 | ATLVT012XX0524 | 45458 | NGTSGGSSNDNH | 5.1 | 5.92 | 1.27 | 2.91 |
| ATLVT012XX0479 | 45413 | NGTAGGSTNDNH | 5.11 | 7.63 | −0.03 | 3.07 | ATLVT012XX0525 | 45459 | NGTSGGSSNDNT | 8.4 | 10.5 | 1.73 | 4.85 |
| ATLVT012XX0480 | 45414 | NGTAGGSTNDNT | 9.3 | 10.09 | 0.43 | 5.26 | ATLVT012XX0526 | 45460 | NGTSGGSTNDNA | 7.25 | 8.83 | 1.85 | 4.2 |
| ATLVT012XX0481 | 45415 | NGTSAAASNDNA | 0.9 | 1.8 | −2.78 | −1.83 | ATLVT012XX0527 | 45461 | NGTSGGSTNDNH | 6.45 | 7.14 | 1.24 | 3.86 |
| ATLVT012XX0482 | 45416 | NGTSAAASNDNH | −0.6 | 1.15 | −1.44 | 0.71 | ATLVT012XX0528 | 45462 | NGTSGGSTNDNT | 9.49 | 11.91 | 1.72 | 5.11 |
| ATLVT012XX0483 | 45417 | NGTSAAASNDNT | −0.04 | 0.08 | −0.15 | 1.58 | ATLVT012XX0529 | 45463 | NGTTAAASNDNA | 1. | 3.03 | 2.15 | 1.56 |
| ATLVT012XX0484 | 45418 | NGTSAAATNDNA | −1.53 | −0.01 | −0.54 | 0.66 | ATLVT012XX0530 | 45464 | NGTTAAASNDNH | −0.26 | 2.95 | 0.97 | 0.68 |
| ATLVT012XX0485 | 45419 | NGTSAAATNDNH | −0.59 | −0.07 | 0.58 | 0.81 | ATLVT012XX0531 | 45465 | NGTTAAASNDNT | −0.17 | 0.06 | 0.79 | 0.19 |
| ATLVT012XX0486 | 45420 | NGTSAAATNDNT | 0.82 | 0.02 | −0.28 | 1.36 | ATLVT012XX0532 | 45466 | NGTTAAATNDNA | 1.75 | 0.54 | 0.46 | 0.7 |
| ATLVT012XX0487 | 45421 | NGTSAASSNDNA | 1.35 | 1.02 | −1.51 | −0.01 | ATLVT012XX0533 | 45467 | NGTTAAATNDNH | 2.51 | 1.08 | 1.32 | 2.27 |
| ATLVT012XX0488 | 45422 | NGTSAASSNDNH | −0.41 | −0.64 | −0.77 | −0.06 | ATLVT012XX0534 | 45468 | NGTTAAATNDNT | 4.07 | 0.75 | 0.24 | 1.78 |
| ATLVT012XX0489 | 45423 | NGTSAASSNDNT | 1.71 | 0.14 | −0.07 | −0.18 | ATLVT012XX0535 | 45469 | NGTTAASSNDNA | −0.54 | −0.95 | 0.58 | −0.84 |
| ATLVT012XX0490 | 45424 | NGTSAASTNDNA | 1.31 | 1.72 | 0.78 | −0.14 | ATTVT012XX0536 | 45470 | NGTTAASSNDNH | 0.59 | 0.36 | 0.92 | 0.39 |
| ATLVT012XX0491 | 45425 | NGTSAASTNDNH | −0.55 | 3.83 | −0.85 | 1.19 | ATLVT012XX0537 | 45471 | NGTTAASSNDNT | 1.84 | 2.43 | 0.97 | 1.02 |
| ATLVT012XX0492 | 45426 | NGTSAASTNDNT | 0.78 | 0.35 | 0.02 | 0.26 | ATLVT012XX0538 | 45472 | NGTTAASTNDNA | −1.03 | 0.58 | −0.27 | 0.68 |
| ATLVT012XX0493 | 45427 | NGTSAGASNDNA | 1.51 | 4.07 | 0.55 | 0.91 | ATLVT012XX0539 | 45473 | NGTTAASTNDNH | −1.15 | 1.68 | 0.7 | 0.54 |
| ATLVT012XX0494 | 45428 | NGTSAGASNDNH | 0.31 | 1.8 | 0.12 | 0.96 | ATLVT012XX0540 | 45474 | NGTTAASTNDNT | 0.05 | 0.82 | 0.86 | 1.2 |
| ATLVT012XX0495 | 45429 | NGTSAGASNDNT | 1.06 | 1.63 | 1.06 | 2.54 | ATLVT012XX0541 | 45475 | NGTTAGASNDNA | 1.18 | 1.47 | 0.5 | 2.89 |
| ATLVT012XX0496 | 45430 | NGTSAGATNDNA | −1.05 | 0.83 | 1.01 | 0.1 | ATLVT012XX0542 | 45476 | NGTTAGASNDNH | 0.83 | 0.99 | 0.91 | 2.79 |
| ATLVT012XX0497 | 45431 | NGTSAGATNDNH | 0.88 | 4.41 | −0.82 | 2.45 | ATLVT012XX0543 | 45477 | NGTTAGASNDNT | 1.46 | 2.16 | 0.99 | 2.19 |
| ATLVT012XX0498 | 45432 | NGTSAGATNDNT | 0.86 | 1.23 | 0.6 | 1.4 | ATLVT012XX0544 | 45478 | NGTTAGATNDNA | −2.1 | 1.4 | 0.77 | 2.04 |
| ATLVT012XX0499 | 45433 | NGTSAGSSNDNA | 0.09 | 0.73 | 0.24 | 1.82 | ATLVT012XX0545 | 45479 | NGTTAGATNDNH | 0.22 | 1.5 | 1.38 | 3.46 |
| ATLVT012XX0500 | 45434 | NGTSAGSSNDNH | −0.58 | 0.14 | 0.55 | −0.51 | ATLVT012XX0546 | 45480 | NGTTAGATNDNT | −0.4 | 0.82 | 0.33 | 3.12 |
| ATLVT012XX0501 | 45435 | NGTSAGSSNDNT | 2.83 | 3.83 | 1.66 | 3.56 | ATLVT012XX0547 | 45481 | NGTTAGSSNDNA | 1.65 | 1.23 | −0.01 | 2.25 |
| ATLVT012XX0502 | 45436 | NGTSAGSTNDNA | 2.69 | 2.06 | 0.73 | 0.87 | ATLVT012XX0548 | 45482 | NGTTAGSSNDNH | 0.1 | −1.12 | 0.21 | 0.06 |
| ATLVT012XX0503 | 45437 | NGTSAGSTNDNH | 2.15 | 2.73 | 0.16 | 1.7 | ATLVT012XX0549 | 45483 | NGTTAGSSNDNT | 1.51 | 1.14 | 0.54 | 2.76 |
| ATLVT012XX0504 | 45438 | NGTSAGSTNDNT | 0.7 | 1.7 | 0.93 | 2.36 | ATLVT012XX0550 | 45484 | NGTTAGSTNDNA | 0.91 | 1.89 | 1.35 | 1.96 |
| ATLVT012XX0505 | 45439 | NGTSGAASNDNA | −0.95 | −1.95 | −1.02 | −0.22 | ATLVT012XX0551 | 45485 | NGTTAGSTNDNH | 1.16 | 1.06 | −0.05 | 2.1 |
| ATLVT012XX0506 | 45440 | NGTSGAASNDNH | 0.06 | −0.27 | 1.06 | −0.5 | ATLVT012XX0552 | 45486 | NGTTAGSTNDNT | 3.38 | 2.98 | 1.39 | 3.38 |
| ATLVT012XX0553 | 45487 | NGTTGAASNDNA | 0.11 | −0.72 | 0.05 | −0.41 | ATTVT012XX0599 | 45533 | NSAAAGSTNDNH | −1.11 | −0.77 | −0.7 | 1.18 |
| ATLVT012XX0554 | 454SS | NGTTGAASNDNH | −1.31 | −0.62 | 1.51 | −0.43 | ATLVT012XX0600 | 45534 | NSAAAGSTNDNT | 1.07 | −0.16 | −1.71 | −0.21 |
| ATLVT012XX0555 | 45489 | NGTTGAASNDNT | −0.99 | 1.45 | −0.54 | −0.89 | ATLVT012XX0601 | 45535 | NSAAGAASNDNA | −3.94 | 1.93 | −2.11 | −3.39 |
| ATLVT012XX0556 | 45490 | NGTTGAATNDNA | −0.95 | 0.09 | −0.09 | 0.36 | ATLVT012XX0602 | 45536 | NSAAGAASNDNH | 0.32 | 4.31 | 0.16 | 0.51 |
| ATLVT012XX0557 | 45491 | NGTTGAATNDNH | −0.16 | 2.12 | −0.59 | 1.8 | ATLVT012XX0603 | 45537 | NSAAGAASNDNT | −3.09 | 1.2 | −2.86 | −1.64 |
| ATLVT012XX0558 | 45492 | NGTTGAATNDNT | −0.01 | 1.91 | −0.12 | 0.53 | ATLVT012XX0604 | 45538 | NSAAGAATNDNA | −1.62 | 3.23 | −1.59 | −0.73 |
| ATLVT012XX0559 | 45493 | NGTTGASSNDNA | 0.51 | 3.92 | 1.86 | 0.27 | ATLVT012XX0605 | 45539 | NSAAGAATNDNH | 1.39 | 5.39 | 0.63 | 2.63 |
| ATLVT012XX0560 | 45494 | NGTTGASSNDNH | 0.85 | 1.65 | 0.48 | 1.46 | ATLVT012XX0606 | 45540 | NSAAGAATNDNT | −4.51 | −0.23 | −1.06 | −1.9 |
| ATLVT012XX0561 | 45495 | NGTTGASSNDNT | 2.2 | 2.44 | 0.11 | 2.28 | ATLVT012XX0607 | 45541 | NSAAGASSNDNA | −4.52 | −0.09 | −1.57 | −1.71 |
| ATLVT012XX0562 | 45496 | NGTTGASTNDNA | 0.03 | 0.76 | 1.22 | 1.79 | ATLVT012XX0608 | 45542 | NSAAGASSNDNH | −2.61 | −1.41 | −2.01 | −1.84 |
| ATLVT012XX0563 | 45497 | NGTTGASTNDNH | 1.35 | 1.1 | 0.16 | 0.77 | ATLVT012XX0609 | 45543 | NSAAGASSNDNT | −3.63 | −0.73 | −1.56 | −1.87 |
| ATLVT012XX0564 | 45498 | NGTTGASTNDNT | 1.34 | −0.5 | 0.54 | 2.01 | ATLVT012XX0610 | 45544 | NSAAGASTNDNA | −0.13 | 0.65 | −1.51 | −0.38 |
| ATLVT012XX0565 | 45499 | NGTTGGASNDNA | 2.04 | 2.98 | 2.07 | 3.78 | ATLVT012XX0611 | 45545 | NSAAGASTNDNH | −1.34 | 0.15 | −1.66 | −1.15 |
| ATLVT012XX0566 | 45500 | NGTTGGASNDNH | 2.68 | 6.21 | 1.31 | 3.59 | ATLVT012XX0612 | 45546 | NSAAGASTNDNT | −0.36 | 0.58 | −1.22 | −1.69 |
| ATLVT012XX0567 | 45501 | NGTTGGASNDNT | 3.83 | 6.47 | 1.36 | 4.46 | ATLVT012XX0613 | 45547 | NSAAGGASNDNA | −1.94 | −0.13 | −2.01 | −1.98 |
| ATLVT012XX0568 | 45502 | NGTTGGATNDNA | 4.38 | 3.99 | 1.34 | 3.44 | ATLVT012XX0614 | 45548 | NSAAGGASNDNH | −0.26 | 1.5 | −1.74 | −0.94 |
| ATLVT012XX0569 | 45503 | NGTTGGATNDNH | 4.93 | 6.02 | 1.39 | 3.69 | ATLVT012XX0615 | 45549 | NSAAGGASNDNT | −0.72 | 1.47 | −1.22 | −0.85 |
| ATLVT012XX0570 | 45504 | NGTTGGATNDNT | 3.62 | 6.59 | 1.16 | 3.95 | ATLVT012XX0616 | 45550 | NSAAGGATNDNA | −3.7 | −0.69 | −2.42 | −0.55 |
| ATLVT012XX0571 | 45505 | NGTTGGSSNDNA | 5.88 | 7.98 | 0.95 | 4.78 | ATLVT012XX0617 | 45551 | NSAAGGATNDNE | 0.71 | 5.32 | −1.45 | 2.03 |
| ATLVT012XX0572 | 45506 | NGTTGGSSNDNH | 3.85 | 4 | 0.15 | 3.35 | ATLVT012XX0618 | 45552 | NSAAGGATNDNT | −0.27 | 4.06 | −1.89 | −0.47 |
| ATLVT012XX0573 | 45507 | NGTTGGSSNDNT | 7.33 | 9.68 | 1.6 | 4.74 | ATLVT012XX0619 | 45553 | NSAAGGSSNDNA | −0.76 | 1 | −0.79 | 0.39 |
| ATLVT012XX0574 | 45508 | NGTTGGSTNDNA | 6.86 | 8.55 | 1.77 | 4.15 | ATLVT012XX0620 | 45554 | NSAAGGSSNDNH | −0.36 | −0.64 | −1.37 | 1.25 |
| ATLVT012XX0575 | 45509 | NGTTGGSTNDNH | 4.83 | 5.76 | 0.69 | 3.65 | ATLVT012XX0621 | 45555 | NSAAGGSSNDNT | 0.96 | 1.93 | −1.25 | 0.09 |
| ATLVT012XX0576 | 45510 | NGTTGGSTNDNT | 8.35 | 9.78 | 1.72 | 5.03 | ATLVT012XX0622 | 45556 | NSAAGGSTNDNA | 0.67 | 3.52 | −0.12 | 0.71 |
| ATLVT012XX0577 | 45511 | NSAAAAASNDNA | −2.46 | −0.44 | 0.32 | 0.65 | ATLVT012XX0623 | 45557 | NSAAGGSTNDNH | −0.73 | 4.31 | 0.29 | 1.22 |
| ATLVT012XX0578 | 45512 | NSAAAAASNDNH | 0.59 | 2.03 | 1.19 | −1.01 | ATLVT012XX0624 | 45558 | NSAAGGSTNDNT | 0.18 | 1.75 | −0.06 | 3.68 |
| ATLVT012XX0579 | 45513 | NSAAAAASNDNT | −2.8 | 2.17 | −0.25 | −1.16 | ATLVT012XX0625 | 45559 | NSASAAASNDNA | −0.35 | 0.99 | −0.25 | −0.11 |
| ATLVT012XX0580 | 45514 | NSAAAAATNDNA | −1.67 | 1.81 | 1.39 | 1.46 | ATLVT012XX0626 | 45560 | NSASAAASNDNH | −1.03 | 0.7 | −0.89 | 0.8 |
| ATLVT012XX0581 | 45515 | NSAAAAATNDNR | −0.38 | 1.42 | 0.38 | 0.22 | ATLVT012XX0627 | 45561 | NSASAAASNDNT | 3.65 | −0.57 | −0.04 | 0.38 |
| ATLVT012XX0582 | 45516 | NSAAAAATNDNT | −1.1 | 2.03 | 0.37 | 0.98 | ATLVT012XX0628 | 45562 | NSASAAATNDNA | 0.09 | 4.89 | 0.45 | 1.82 |
| ATLVT012XX0583 | 45517 | NSAAAASSNDNA | −0.32 | −1.71 | −0.24 | −0.69 | ATLVT012XX0629 | 45563 | NSASAAATNDNH | −0.29 | 3.59 | −0.22 | 0.98 |
| ATLVT012XX0584 | 45518 | NSAAAASSNDNH | 2.65 | 2.38 | 0.4 | −0.86 | ATLVT012XX0630 | 45564 | NSASAAATNDNT | 0.7 | 0.36 | 0.49 | −0.14 |
| ATLVT012XX0585 | 45519 | NSAAAASSNDNT | 2.71 | −1.41 | −0.39 | 0.22 | ATLVT012XX0631 | 45565 | NSASAASSNDNA | −1.65 | −1.5 | 0.78 | −0.45 |
| ATLVT012XX0586 | 45520 | NSAAAASTNDNA | 1.32 | 1.7 | −0.58 | .1.13 | ATLVT012XX0632 | 45566 | NSASAASSNDNH | −1.02 | 0.55 | 1.39 | −1.41 |
| ATLVT012XX0587 | 45521 | NSAAAASTNDNH | 0.14 | 0.54 | 0.19 | −0.23 | ATLVT012XX0633 | 45567 | NSASAASSNDNT | −0.12 | −1.15 | 0.02 | −1.29 |
| ATLVT012XX0588 | 45522 | NSAAAASTNDNT | −0.34 | 1.12 | −0.07 | −1.28 | ATLVT012XX0634 | 45568 | NSASAASTNDNA | 1.63 | 1.06 | 0.83 | −0.52 |
| ATLVT012XX0589 | 45523 | NSAAAGASNDNA | 3.09 | −0.95 | −1.34 | −1.57 | ATLVT012XX0635 | 45569 | NSASAASTNDNH | −0.11 | .1.24 | −0.35 | −0.49 |
| ATLVT012XX0590 | 45524 | NSAAAGASNDNH | −2.38 | −1.94 | −1.04 | −0.32 | ATLVT012XX0636 | 45570 | NSASAASTNDNT | 0.89 | −1.73 | 0.77 | 1.16 |
| ATLVT012XX0591 | 45525 | NSAAAGASNDNT | −2.16 | −0.79 | −0.25 | −2.09 | ATLVT012XX0637 | 45571 | NSASAGASNDNA | −1.57 | −1.54 | 0.22 | −1.11 |
| ATLVT012XX0592 | 45526 | NSAAAGATNDNA | −1.67 | −2.1 | −1.31 | 0.08 | ATLVT012XX0638 | 45572 | NSASAGASNDNH | −0.71 | 1.24 | −1 | 0.42 |
| ATLVT012XX0593 | 45527 | NSAAAGATNDNH | −0.74 | −1.03 | 1.47 | 2.29 | ATLVT012XX0639 | 45573 | NSASAGASNDNT | −0.3 | −1.2 | −0.23 | 1.46 |
| ATLVT012XX0594 | 45528 | NSAAAGATNDNT | −1.39 | −2.45 | −0.18 | 1.8 | ATLVT012XX0640 | 45574 | NSASAGATNDNA | 0.06 | −0.01 | 0.87 | 1.33 |
| ATLVT012XX0595 | 45529 | NSAAAGSSNDNA | −0.7 | −2.96 | −1.7 | −0.78 | ATLVT012XX0641 | 45575 | NSASAGATNDNH | −0.44 | 1.87 | 1.05 | 1.92 |
| ATLVT012XX0596 | 45530 | NSAAAGSSNDNH | −0.24 | −2.38 | −1.44 | 0.88 | ATLVT012XX0642 | 45576 | NSASAGATNDNT | −0.26 | −1.05 | −0.52 | 1.36 |
| ATLVT012XX0597 | 45531 | NSAAAGSSNDNT | −1.44 | −1.59 | −0.16 | −2.24 | ATLVT012XX0643 | 45577 | NSASAGSSNDNA | −0.89 | 0.29 | −1.48 | 0.44 |
| ATLVT012XX0598 | 45532 | NSAAAGSTNDNA | −1.71 | −1.08 | −1.15 | 0.15 | ATLVT012XX0644 | 45578 | NSASAGSSNDNH | −0.61 | 3.2 | 0.01 | −1.12 |
| ATLVT012XX0645 | 45579 | NSASAGSSNDNT | 0.61 | 0.73 | 0.77 | 0.63 | ATLVT012XX0691 | 45625 | NSATAGSSNDNA | 1.34 | 1.38 | −0.4 | −0.18 |
| ATLVT012XX0646 | 45580 | NSASAGSTNDNA | 1.73 | 1.33 | −0.08 | −0.53 | ATLVT012XX0692 | 45626 | NSATAGSSNDNH | −0.23 | 0.5 | 0.26 | 0.29 |
| ATLVT012XX0647 | 45581 | NSASAGSTNDNH | 1.55 | 1.83 | 0.96 | 0.78 | ATLVT012XX0693 | 45627 | NSATAGSSNDNT | 0.19 | 4.99 | 0.31 | −0.12 |
| ATLVT012XX0648 | 45582 | NSASAGSTNDNT | 1.63 | −0.37 | 0.26 | 0.48 | ATLVT012XX0694 | 45628 | NSATAGSTNDNA | 1.81 | 1.5 | 0.37 | 0.38 |
| ATLVT012XX0649 | 45583 | NSASGAASNDNA | −0.6 | 1.53 | −2.28 | 0.02 | ATLVT012XX0695 | 45629 | NSATAGSTNDNH | −0.88 | 2.49 | −0.06 | 1.44 |
| ATLVT012XX0650 | 45584 | NSASGAASNDNH | −1.52 | 0.71 | −0.97 | −0.27 | ATLVT012XX0696 | 45630 | NSATAGSTNDNT | −0.39 | 2.04 | −0.92 | 1.24 |
| ATLVT012XX0651 | 45585 | NSASGAASNDNT | 0.04 | 1.57 | −0.52 | −0.91 | ATLVT012XX0697 | 45631 | NSATGAASNDNA | 0.28 | −2.03 | −0.45 | −0.33 |
| ATLVT012XX0652 | 45586 | NSASGAATNDNA | −2.73 | 1.04 | −0.54 | −2.52 | ATLVT012XX0698 | 45632 | NSATGAASNDNH | 2.72 | −0.2 | 0.95 | 0.5 |
| ATLVT012XX0653 | 45587 | NSASGAATNDNH | −0.7 | 4.05 | 0.1 | 0.2 | ATTVT012XX0699 | 45633 | NSATGAASNDNT | 1.2 | −3.01 | −0.64 | −2.36 |
| ATLVT012XX0654 | 455SS | NSASGAATNDNT | −1.98 | 0.96 | −0.81 | −0.8 | ATLVT012XX0700 | 45634 | NSATGAATNDNA | −0.36 | −1.01 | −1.91 | −0.56 |
| ATLVT012XX0655 | 45589 | NSASGASSNDNA | −0.22 | 0.8 | 1.33 | −1.85 | ATLVT012XX0701 | 45635 | NSATGAATNDNH | 1.4 | 1.99 | 0.02 | 0.56 |
| ATLVT012XX0656 | 45590 | NSASGASSNDNH | 2.46 | 0.28 | 0.48 | −0.88 | ATLVT012XX0702 | 45636 | NSATGAATNDNT | −1.33 | −2.24 | −1.02 | −0.88 |
| ATLVT012XX0657 | 45591 | NSASGASSNDNT | 0.36 | 4.28 | 0.43 | −0.04 | ATLVT012XX0703 | 45637 | NSATGASSNDNA | −0.9 | −1.85 | 0.08 | 0.01 |
| ATLVT012XX0658 | 45592 | NSASGASTNDNA | −0.29 | 1.5 | −0.01 | −1.47 | ATLVT012XX0704 | 45638 | NSATGASSNDNH | 0.98 | −1.42 | 0.84 | 1.15 |
| ATLVT012XX0659 | 45593 | NSASGASTNDNH | 0.38 | 0.58 | 0.61 | −0.22 | ATLVT012XX0705 | 45639 | NSATGASSNDNT | 0.48 | −2.5 | −0.32 | 0.94 |
| ATLVT012XX0660 | 45594 | NSASGASTNDNT | 1.18 | −0.22 | 1.04 | 0.07 | ATLVT012XX0706 | 45640 | NSATGASTNDNA | −0.02 | −0.93 | 0.04 | 1.26 |
| ATLVT012XX0661 | 45595 | NSASGGASNDNA | 0.04 | 0.69 | −0.45 | 0.23 | ATTVT012XX0707 | 45641 | NSATGASTNDNH | −1.29 | 0.86 | 0.08 | 0.83 |
| ATLVT012XX0662 | 45596 | NSASGGASNDNH | 1.48 | −0.6 | −0.39 | 1.57 | ATLVT012XX0708 | 45642 | NSATGASTNDNT | 0.06 | −1.82 | 0.04 | −0.52 |
| ATLVT012XX0663 | 45597 | NSASGGASNDNT | 1.82 | 2.16 | 0.28 | 1.49 | ATLVT012XX0709 | 45643 | NSATGGASNDNA | 0.55 | 0.23 | 0.13 | 0.32 |
| ATLVT012XX0664 | 45598 | NSASGGATNDNA | 0.75 | 2.47 | −0.12 | 2.84 | ATLVT012XX0710 | 45644 | NSATGGASNDNH | 0.08 | 0.3 | 1.51 | 2.69 |
| ATLVT012XX0665 | 45599 | NSASGGATNDNH | 4.45 | 2.87 | 0.77 | 3.06 | ATLVT012XX0711 | 45645 | NSATGGASNDNT | 0.08 | 0.61 | 0.65 | 1.45 |
| ATLVT012XX0666 | 45600 | NSASGGATNDNT | 2.39 | 1.69 | 0.15 | 2.5 | ATTVT012XX0712 | 45646 | NSATGGATNDNA | 1.02 | 1.16 | −0.2 | 2.53 |
| ATLVT012XX0667 | 45601 | NSASGGSSNDNA | 1.1 | 1.95 | 0.4 | 1.65 | ATLVT012XX0713 | 45647 | NSATGGATNDNH | 3.59 | 4.03 | 0.05 | 3.78 |
| ATLVT012XX0668 | 45602 | NSASGGSSNDNH | 1.42 | 1.62 | 0.91 | 1.23 | ATLVT012XX0714 | 45648 | NSATGGATNDNT | 2.11 | 2.98 | −0.14 | 2.02 |
| ATLVT012XX0669 | 45603 | NSASGGSSNDNT | 2.63 | 1.49 | 0.02 | 1.69 | ATLVT012XX0715 | 45649 | NSATGGSSNDNA | 2.21 | 1.23 | 0.72 | 1.3 |
| ATLVT012XX0670 | 45604 | NSASGGSTNDNA | 2.55 | 3.83 | −0.28 | 0.93 | ATLVT012XX0716 | 45650 | NSATGGSSNDNH | 1.89 | −0.01 | 0.09 | −0.07 |
| ATLVT012XX0671 | 45605 | NSASGGSTNDNH | 2.73 | 1.39 | 0.45 | 2.67 | ATLVT012XX0717 | 45651 | NSATGGSSNDNT | 2.18 | 1.31 | 0.99 | 1.42 |
| ATLVT012XX0672 | 45606 | NSASGGSTNDNT | 4.34 | 2.6 | 0.68 | 2.67 | ATLVT012XX0718 | 45652 | NSATGGSTNDNA | 0.86 | 1.5 | 0.45 | 1.98 |
| ATLVT012XX0673 | 45607 | NSATAAASNDNA | −0.98 | −0.47 | −0.27 | −1.27 | ATLVT012XX0719 | 45653 | NSATGGSTNDNH | 1.31 | 3.29 | 0.55 | 1.88 |
| ATLVT012XX0674 | 45608 | NSATAAASNDNH | −0.61 | 0.9 | −0.02 | −0.4 | ATLVT012XX0720 | 45654 | NSATGGSTNDNT | 2.94 | 1.25 | 1.13 | 0.78 |
| ATLVT012XX0675 | 45609 | NSATAAASNDNT | 0.27 | −1.46 | 0.26 | 0.53 | ATLVT012XX0721 | 45655 | NSEAAAASNDNA | −1.1 | −1.33 | 0.6 | −0.21 |
| ATLVT012XX0676 | 45610 | NSATAAATNDNA | −1.99 | 0.43 | −0.51 | −0.95 | ATLVT012XX0722 | 45656 | NSEAAAASNDNH | −3.13 | −1.28 | 0.42 | −1.94 |
| ATLVT012XX0677 | 45611 | NSATAAATNDNH | .1.28 | −1.5 | −0.94 | 2.58 | ATLVT012XX0723 | 45657 | NSEAAAASNDNT | −2.11 | 1.42 | 0.33 | −2.44 |
| ATLVT012XX0678 | 45612 | NSATAAATNDNT | −0.91 | −2.38 | 0.39 | −1.1 | ATLVT012XX0724 | 45658 | NSEAAAATNDNA | −2.39 | −1.11 | −0.31 | −1.49 |
| ATLVT012XX0679 | 45613 | NSATAASSNDNA | −1.49 | 0.31 | −0.23 | −0.27 | ATLVT012XX0725 | 45659 | NSEAAAATNDNH | −0.84 | 1.6 | −0.43 | 1.45 |
| ATLVT012XX0680 | 45614 | NSATAASSNDNH | −1.23 | −1.21 | 0.72 | −1.55 | ATLVT012XX0726 | 45660 | NSEAAAATNDNT | −0.15 | −0.89 | −0.21 | 0.49 |
| ATLVT012XX0681 | 45615 | NSATAASSNDNT | 0.22 | −0.19 | −0.16 | 0.62 | ATLVT012XX0727 | 45661 | NSEAAASSNDNA | 1.47 | 0.84 | −0.12 | −1.84 |
| ATLVT012XX0682 | 45616 | NSATAASTNDNA | 1.63 | 2.16 | 0.1 | −1.32 | ATLVT012XX0728 | 45662 | NSEAAASSNDNH | 2.06 | 1.07 | 1.46 | −3.01 |
| ATLVT012XX0683 | 45617 | NSATAASTNDNH | 1.44 | 1.27 | 0.84 | −0.18 | ATLVT012XX0729 | 45663 | NSEAAASSNDNT | −0.14 | −1.51 | −0.68 | 0.2 |
| ATLVT012XX0684 | 45618 | NSATAASTNDNT | 0.51 | 4.9 | 0.28 | 0.45 | ATLVT012XX0730 | 45664 | NSEAAASTNDNA | −0.57 | 0.79 | 0 | −1.36 |
| ATLVT012XX0685 | 45619 | NSATAGASNDNA | 2.8 | 2.68 | −1.23 | 1.26 | ATLVT012XX0731 | 45665 | NSEAAASTNDNH | −1.51 | −1.49 | 0.94 | −1.78 |
| ATLVT012XX0686 | 45620 | NSATAGASNDNH | 3.83 | 2.2 | 0.24 | 1.92 | ATLVT012XX0732 | 45666 | NSEAAASTNDNT | −0.87 | 1.36 | 0.03 | −1.68 |
| ATLVT012XX0687 | 45621 | NSATAGASNDNT | 1.5 | 1.38 | −1.79 | 0.26 | ATLVT012XX0733 | 45667 | NSEAAGASNDNA | −2.77 | −0.12 | −2.15 | −2.84 |
| ATLVT012XX0688 | 45622 | NSATAGATNDNA | 1.78 | −0.06 | 1.17 | 1.19 | ATLVT012XX0734 | 45668 | NSEAAGASNDNH | −1.07 | −0.51 | −0.6 | −2.56 |
| ATLVT012XX0689 | 45623 | NSATAGATNDNH | 2.75 | 2.91 | 0.75 | 3.35 | ATLVT012XX0735 | 45669 | NSEAAGASNDNT | −2.42 | −1.61 | −3.66 | −2.59 |
| ATLVT012XX0690 | 45624 | NSATAGATNDNT | 1.64 | −0.17 | −0.03 | 2.23 | ATLVT012XX0736 | 45670 | NSEAAGATNDNA | −1.28 | 0.74 | −0.93 | −0.88 |
| ATLVT012XX0737 | 45671 | NSEAAGATNDNH | −0.1 | 2.04 | −0.97 | −0.72 | ATTVT012XX0783 | 45717 | NSESAGASNDNT | −1.23 | −0.61 | −1.37 | −2.26 |
| ATLVT012XX0738 | 45672 | NSEAAGATNDNT | −0.07 | 0.05 | −1.15 | −1.29 | ATLVT012XX0784 | 45718 | NSESAGATNDNA | −1.9 | −2.74 | 0.63 | −0.99 |
| ATLVT012XX0739 | 45673 | NSEAAGSSNDNA | −0.15 | −0.53 | −1.13 | −2.19 | ATLVT012XX0785 | 45719 | NSESAGATNDNH | −0.05 | −0.21 | 0.43 | 2.52 |
| ATLVT012XX0740 | 45674 | NSEAAGSSNDNH | −0.5 | −2.47 | −0.64 | −3.29 | ATLVT012XX0786 | 45720 | NSESAGATNDNT | −1.24 | −3 | −0.31 | −1.34 |
| ATLVT012XX0741 | 45675 | NSEAAGSSNDNT | 1.48 | −2.11 | −1.37 | −0.45 | ATLVT012XX0787 | 45721 | NSESAGSSNDNA | −0.81 | −2.11 | −0.44 | −2.38 |
| ATLVT012XX0742 | 45676 | NSEAAGSTNDNA | 0.54 | 0.33 | −0.16 | −0.18 | ATLVT012XX0788 | 45722 | NSESAGSSNDNH | −0.6 | 0.09 | 0.33 | −0.54 |
| ATLVT012XX0743 | 45677 | NSEAAGSTNDNH | 3.8 | −1.72 | −0.23 | 0.94 | ATLVT012XX0789 | 45723 | NSESAGSSNDNT | 1.72 | −1.7 | −0.8 | −0.36 |
| ATLVT012XX0744 | 45678 | NSEAAGSTNDNT | 1.13 | −1.54 | −0.92 | 0.7 | ATLVT012XX0790 | 45724 | NSESAGSTNDNA | −0.56 | −0.09 | 0.52 | −2.04 |
| ATLVT012XX0745 | 45679 | NSEAGAASNDNA | 0.01 | 1.04 | 1.03 | −3.04 | ATLVT012XX0791 | 45725 | NSESAGSTNDNH | −0.91 | −1.48 | −0.68 | 0.78 |
| ATLVT012XX0746 | 45680 | NSEAGAASNDNH | −0.31 | −1.98 | 1.18 | −2.01 | ATLVT012XX0792 | 45726 | NSESAGSTNDNT | −1.89 | −0.2 | 0.48 | −2.55 |
| ATLVT012XX0747 | 45681 | NSEAGAASNDNT | −1.93 | −2.55 | −0.68 | −0.87 | ATLVT012XX0793 | 45727 | NSESGAASNDNA | −1.82 | −0.29 | −0.3 | −2.47 |
| ATLVT012XX0748 | 45682 | NSEAGAATNDNA | −2.27 | −1.08 | −1.2 | 0.6 | ATLVT012XX0794 | 45728 | NSESGAASNDNH | −0.24 | −2.24 | −2.42 | −2.67 |
| ATLVT012XX0749 | 45683 | NSEAGAATNDNH | −0.67 | 1.58 | −1.07 | 0.82 | ATLVT012XX0795 | 45729 | NSESGAASNDNT | −1.71 | −3.22 | −0.67 | −0.06 |
| ATLVT012XX0750 | 45684 | NSEAGAATNDNT | −0.3 | −0.33 | −1.23 | −1.97 | ATLVT012XX0796 | 45730 | NSESGAATNDNA | −0.55 | −2.35 | −1.64 | −0.19 |
| ATLVT012XX0751 | 45685 | NSEAGASSNDNA | −0.34 | −1.24 | −0.35 | 0.81 | ATLVT012XX0797 | 45731 | NSESGAATNDNH | −1.11 | −1.8 | −1.46 | −0.96 |
| ATLVT012XX0752 | 45686 | NSEAGASSNDNH | −0.58 | −1.58 | −0.68 | −1.66 | ATLVT012XX0798 | 45732 | NSESGAATNDNT | −1.41 | −2.06 | −0.57 | −0.62 |
| ATLVT012XX0753 | 45687 | NSEAGASSNDNT | 0.64 | −1.78 | 0.47 | 0.07 | ATLVT012XX0799 | 45733 | NSESGASSNDNA | −0.67 | −1.08 | −0.03 | −1.61 |
| ATLVT012XX0754 | 45688 | NSEAGASTNDNA | 0.16 | −2.95 | −0.45 | −0.87 | ATLVT012XX0800 | 45734 | NSESGASSNDNH | 0.59 | −2.62 | −0.56 | −1.9 |
| ATLVT012XX0755 | 45689 | NSEAGASTNDNH | 0.7 | −2.47 | −0.05 | −1.36 | ATLVT012XX0801 | 45735 | NSESGASSNDNT | 0.33 | −1.85 | −0.3 | −1.25 |
| ATLVT012XX0756 | 45690 | NSEAGASTNQNT | 0.56 | −3.23 | 0.7 | −1.68 | ATLVT012XX0802 | 45736 | NSESGASTNDNA | 2.25 | 2.55 | 0.15 | −0.66 |
| ATLVT012XX0757 | 45691 | NSEAGGASNDNA | −0.06 | 3.3 | −1.37 | −0.29 | ATLVT012XX0803 | 45737 | NSESGASTNDNH | 0.89 | −0.84 | −0.45 | −0.65 |
| ATLVT012XX0758 | 45692 | NSEAGGASNDNH | −0.26 | 1.2 | 0.72 | −0.24 | ATTVT012XX0804 | 45738 | NSESGASTNDNT | 1.24 | 0.06 | 0.26 | −0.58 |
| ATLVT012XX0759 | 45693 | NSEAGGASNDNT | 0.14 | 0.5 | −2.42 | −1.89 | ATLVT012XX0805 | 45739 | NSESGGASNDNA | 3.33 | 4.52 | 0.42 | 0.28 |
| ATLVT012XX0760 | 45694 | NSEAGGATNDNA | 0.45 | 3.01 | −0.24 | −0.67 | ATLVT012XX0806 | 45740 | NSESGGASNDNH | 1.19 | 0.6 | −0.32 | −0.01 |
| ATLVT012XX0761 | 45695 | NSEAGGATNDNH | 3.82 | 1.81 | 0.26 | 1.42 | ATLVT012XX0807 | 45741 | NSESGGASNDNT | 2.3 | 1.43 | 0.12 | 1.02 |
| ATLVT012XX0762 | 45696 | NSEAGGATNDNT | 3.63 | 1.73 | −1.25 | −0.84 | ATLVT012XX0808 | 45742 | NSESGGATNDNA | −1.15 | 0.28 | 0.38 | −0.55 |
| ATLVT012XX0763 | 45697 | NSEAGGSSNDNA | 1.25 | −1.02 | −0.21 | −1.35 | ATLVT012XX0809 | 45743 | NSESGGATNDNH | 2.4 | 2.19 | −0.12 | 1.57 |
| ATLVT012XX0764 | 45698 | NSEAGGSSNDNH | 3.89 | −1.3 | 0.88 | 0.66 | ATLVT012XX0810 | 45744 | NSESGGATNDNT | 0.4 | 0. | 0.81 | 1.05 |
| ATLVT012XX0765 | 45699 | NSEAGGSSNDNT | 3.16 | −0.7 | −0.34 | 1.58 | ATLVT012XX0811 | 45745 | NSESGGSSNDNA | 0.69 | 1.61 | −0.35 | 2.44 |
| ATLVT012XX0766 | 45700 | NSEAGGSTNDNA | 0.85 | 0.32 | 0.48 | 1.51 | ATLVT012XX0812 | 45746 | NSESGGSSNDNH | −0.63 | 0.26 | 0.22 | 0.26 |
| ATLVT012XX0767 | 45701 | NSEAGGSTNDNH | 1.53 | −0.7 | 0.98 | 1.69 | ATLVT012XX0813 | 45747 | NSESGGSSNDNT | 3.24 | 1.02 | −0.04 | 2.14 |
| ATLVT012XX0768 | 45702 | NSEAGGSTNDNT | 3.12 | 1.27 | 0.11 | 2.38 | ATLVT012XX0814 | 45748 | NSESGGSTNDNA | 1.56 | 2.7 | 1.35 | 1.51 |
| ATLVT012XX0769 | 45703 | NSESAAASNDNA | −1.53 | −0.45 | 0.24 | −2.38 | ATLVT012XX0815 | 45749 | NSESGGSTNDNH | 2.73 | 3.35 | −0.52 | 1.7 |
| ATLVT012XX0770 | 45704 | NSESAAASNDNH | −0.05 | −2.29 | 0.38 | −2.07 | ATLVT012XX0816 | 45750 | NSESGGSTNDNT | 2.97 | 3.62 | 1.08 | 2.49 |
| ATLVT012XX0771 | 45705 | NSESAAASNDNT | −0.72 | −2.37 | 0.18 | −0.03 | ATLVT012XX0817 | 45751 | NSETAAASNDNA | 1.17 | −2.07 | 0.4 | −1.58 |
| ATLVT012XX0772 | 45706 | NSESAAATNDNA | 1.14 | 2.2 | −0.24 | −1.37 | ATLVT012XX0818 | 45752 | NSETAAASNDNH | −0.27 | −0.8 | 0.11 | −0.72 |
| ATLVT012XX0773 | 45707 | NSESAAATNDNH | 1.7 | −1 | −0.35 | 0.59 | ATLVT012XX0819 | 45753 | NSETAAASNDNT | 0.82 | −0.42 | −0.54 | −2.06 |
| ATLVT012XX0774 | 45708 | NSESAAATNDNT | 0.95 | −0.11 | 0.29 | −0.57 | ATLVT012XX0820 | 45754 | NSETAAATNDNA | −1.23 | 1.72 | −0.79 | −0.69 |
| ATLVT012XX0775 | 45709 | NSESAASSNDNA | −1.79 | −2.13 | 0.82 | 0.33 | ATLVT012XX0821 | 45755 | NSETAAATNDNH | 1.37 | −0.54 | 0.16 | −0.75 |
| ATLVT012XX0776 | 45710 | NSESAASSNDNH | −0.98 | −1.76 | 0.45 | −2.11 | ATLVT012XX0822 | 45756 | NSETAAATNDNT | 0.38 | 1.58 | −0.03 | 0.11 |
| ATLVT012XX0777 | 45711 | NSESAASSNDNT | 0.38 | 1.95 | −1.03 | 0.7 | ATLVT012XX0823 | 45757 | NSETAASSNDNA | −1.6 | 0.06 | −0.56 | −1.22 |
| ATLVT012XX0778 | 45712 | NSESAASTNDNA | −2.56 | −2.95 | 0.39 | −1.6 | ATLVT012XX0824 | 45758 | NSETAASSNDNH | 0.97 | −1 | 0.26 | −1.78 |
| ATLVT012XX0779 | 45713 | NSESAASTNDNH | −2.68 | −2.79 | 0.69 | −1.77 | ATLVT012XX0825 | 45759 | NSETAASSNDNT | −0.42 | −0.16 | −0.25 | −3.19 |
| ATLVT012XX0780 | 45714 | NSESAASTNDNT | −0.27 | −2.68 | −1.36 | −0.95 | ATLVT012XX0826 | 45760 | NSETAASTNDNA | −0.47 | 2.1 | 0.02 | −2.96 |
| ATLVT012XX0781 | 45715 | NSESAGASNDNA | −2.38 | −1.56 | −1.29 | −1.33 | ATLVT012XX0827 | 45761 | NSETAASTNDNH | −0.28 | −1.55 | −0.2 | −2.03 |
| ATLVT012XX0782 | 45716 | NSESAGASNDNH | −2.65 | 0.56 | −1.25 | −0.94 | ATLVT012XX0828 | 45762 | NSETAASTNDNT | 1.04 | −1.59 | −1.36 | −2.02 |
| ATLVT012XX0829 | 45763 | NSETAGASNDNA | 0.25 | −0.22 | −0.45 | −2.66 | ATLVT012XX0875 | 45809 | NSQAAASTNDNH | −1.91 | −1.04 | −1.65 | −1.55 |
| ATLVT012XX0830 | 45764 | NSETAGASNDNH | 0.53 | −2.13 | −1.88 | −1.45 | ATLVT012XX0876 | 45810 | NSQAAASTNDNT | −1.48 | −0.9 | −1 | −1.79 |
| ATLVT012XX0831 | 45765 | NSETAGASNDNT | 1.16 | −1.86 | −1.63 | −2.49 | ATLVT012XX0877 | 45811 | NSQAAGASNDNA | 0.55 | −0.82 | −1.46 | −1.83 |
| ATLVT012XX0832 | 45766 | NSETAGATNDNA | 0.16 | −1.28 | 0.18 | −0.71 | ATLVT012XX0878 | 45812 | NSQAAGASNDNH | −1.66 | −2.19 | −1.27 | −3.63 |
| ATLVT012XX0833 | 45767 | NSETAGATNDNH | 0.16 | −1.48 | 0.58 | 0.7 | ATLVT012XX0879 | 45813 | NSQAAGASNDNT | 0.13 | −0.67 | −1.48 | −1.52 |
| ATLVT012XX0834 | 45768 | NSETAGATNDNT | −0.42 | −2.91 | 1.18 | −0.15 | ATLVT012XX0880 | 45814 | NSQAAGATNDNA | −0.19 | −0.03 | −1.15 | −0.35 |
| ATLVT012XX0835 | 45769 | NSETAGSSNDNA | −0.03 | 2.21 | 0.25 | −1.25 | ATLVT012XX0881 | 45815 | NSQAAGATNDNH | 0.35 | −0.21 | −2.38 | −0.78 |
| ATLVT012XX0836 | 45770 | NSETAGSSNDNH | 0.06 | −0.02 | 1.07 | −2.42 | ATLVT012XX0882 | 45816 | NSQAAGATNDNT | −2.13 | −1.82 | −1.03 | −2.86 |
| ATLVT012XX0837 | 45771 | NSETAGSSNDNT | 1.42 | 1.12 | −0.76 | −0.44 | ATLVT012XX0883 | 45817 | NSQAAGSSNDNA | −2.23 | 0.4 | −0.33 | 0.23 |
| ATLVT012XX0838 | 45772 | NSETAGSTNDNA | −2.39 | −1.04 | −0.1 | −1.44 | ATLVT012XX0884 | 45818 | NSQAAGSSNDNH | −4.09 | −2.24 | −1.93 | 0.8 |
| ATLVT012XX0839 | 45773 | NSETAGSTNDNH | −0.46 | 1.11 | 0.02 | −0.01 | ATLVT012XX0885 | 45819 | NSQAAGSSNDNT | −2.76 | −1.19 | −1.99 | 0.43 |
| ATLVT012XX0840 | 45774 | NSETAGSTNDNT | 0.02 | 0.67 | −0.76 | −0.96 | ATLVT012XX0886 | 45820 | NSQAAGSTNDNA | −3.04 | 0.92 | −0.32 | −0.25 |
| ATLVT012XX0841 | 45775 | NSETGAASNDNA | −2.08 | −0.28 | −2.15 | 0.7 | ATLVT012XX0887 | 45821 | NSQAAGSTNDNH | −2.7 | 2.83 | −1.02 | −0.86 |
| ATLVT012XX0842 | 45776 | NSETGAASNDNH | −0.65 | −0.37 | −2.03 | −0.73 | ATLVT012XX0888 | 45822 | NSQAAGSTNDNT | −2.55 | 2.91 | 0.01 | −0.88 |
| ATLVT012XX0843 | 45777 | NSETGAASNDNT | −0.53 | 0.44 | −0.99 | −0.5 | ATLVT012XX0889 | 45823 | NSQAGAASNDNA | −2.51 | 0.47 | −2.15 | −0.81 |
| ATLVT012XX0844 | 45778 | NSETGAATNDNA | −2.44 | 2.37 | −1.48 | −0.04 | ATLVT012XX0890 | 45824 | NSQAGAASNDNH | −4.58 | −2.72 | −1.64 | −1.97 |
| ATLVT012XX0845 | 45779 | NSETGAATNDNH | −0.71 | 0.56 | 0.03 | 2.11 | ATLVT012XX0891 | 45825 | NSQAGAASNDNT | −3.94 | −2.91 | −1.6 | −0.05 |
| ATLVT012XX0846 | 45780 | NSETGAATNDNT | −2.26 | 1.49 | −2.14 | −0.89 | ATLVT012XX0892 | 45826 | NSQAGAATNDNA | −3.23 | −2.78 | 0.9 | −1.29 |
| ATLVT012XX0847 | 45781 | NSETGASSNDNA | 0.11 | 0.71 | −0.1 | −0.34 | ATLVT012XX0893 | 45827 | NSQAGAATNDNH | −3.26 | −2.02 | −0.48 | −0.21 |
| ATLVT012XX0848 | 45782 | NSETGASSNDNH | −1.42 | −0.77 | −0.02 | 0.14 | ATLVT012XX0894 | 45828 | NSQAGAATNDNT | −4.18 | −2.14 | −1.26 | −0.7 |
| ATLVT012XX0849 | 45783 | NSETGASSNDNT | −0.63 | −1.99 | −0.4 | −1.43 | ATLVT012XX0895 | 45829 | NSQAGASSNDNA | −1.28 | 2.04 | −0.39 | −1.98 |
| ATLVT012XX0850 | 45784 | NSETGASTNDNA | −2.01 | −0.73 | 0.34 | −0.93 | ATLVT012XX0896 | 45830 | NSQAGASSNDNH | 2.07 | 2.24 | −0.4 | −1.63 |
| ATLVT012XX0851 | 45785 | NSETGASTNDNH | −2.67 | −1.99 | 0.34 | −0.26 | ATLVT012XX0897 | 45831 | NSQAGASSNDNT | −0.02 | 0.28 | −1.04 | −2.39 |
| ATLVT012XX0852 | 45786 | NSETGASTNDNT | −1.68 | −1.85 | −0.53 | −1.68 | ATLVT012XX0898 | 45832 | NSQAGASTNDNA | −2.52 | −1.9 | −0.3 | −0.48 |
| ATLVT012XX0853 | 45787 | NSETGGASNDNA | −1.59 | 5.32 | −0.77 | 0.96 | ATLVT012XX0899 | 45833 | NSQAGASTNDNH | −1.85 | −0.56 | 0.33 | −1.11 |
| ATLVT012XX0854 | 45788 | NSETGGASNDNH | −1.23 | 2.28 | 0.46 | 2.2 | ATLVT012XX0900 | 45834 | NSQAGASTNDNT | −3.5 | −2.74 | −0.23 | 0.93 |
| ATLVT012XX0855 | 45789 | NSETGGASNDNT | −0.27 | 1.34 | 0.46 | 0.06 | ATLVT012XX0901 | 45835 | NSQAGGASNDNA | −1.02 | −1.59 | −0.3 | 2.11 |
| ATLVT012XX0856 | 45790 | NSETGGATNDNA | 1.48 | 2.65 | −0.51 | 2.43 | ATLVT012XX0902 | 45836 | NSQAGGASNDNH | 0.82 | 1.39 | −0.69 | 1.74 |
| ATLVT012XX0857 | 45791 | NSETGGATNDNH | 2.05 | 2.9 | 0.44 | 1.47 | ATLVT012XX0903 | 45837 | NSQAGGASNDNT | −0.82 | −0.52 | −0.08 | 0.25 |
| ATLVT012XX0858 | 45792 | NSETGGATNDNT | 0.18 | 1.89 | −0.23 | 1.48 | ATLVT012XX0904 | 45838 | NSQAGGATNDNA | −0.74 | −0.05 | −0.32 | 2.11 |
| ATLVT012XX0859 | 45793 | NSETGGSSNDNA | 1.1 | 0.18 | 0.17 | 1.62 | ATLVT012XX0905 | 45839 | NSQAGGATNDNH | 3.34 | 3.97 | 0.72 | 3.67 |
| ATLVT012XX0860 | 45794 | NSETGGSSNDNH | 0.58 | 0.41 | −0.1 | −0.49 | ATLVT012XX0906 | 45840 | NSQAGGATNDNT | 0.49 | 1.57 | 0.02 | 1.94 |
| ATLVT012XX0861 | 45795 | NSETGGSSNDNT | 2.18 | 0.45 | −0.44 | 2.07 | ATLVT012XX0907 | 45841 | NSQAGGSSNDNA | −0.02 | −0.2 | 1.5 | 2.85 |
| ATLVT012XX0862 | 45796 | NSETGGSTNDNA | 2.39 | 0.44 | 0.68 | 1.57 | ATLVT012XX0908 | 45842 | NSQAGGSSNDNH | −0.59 | −1.43 | 1.24 | 0.41 |
| ATLVT012XX0863 | 45797 | NSETGGSTNDNH | 2.75 | 1.13 | 0.29 | 2.82 | ATLVT012XX0909 | 45843 | NSQAGGSSNDNT | 1.27 | 2.13 | 0.47 | 2.13 |
| ATLVT012XX0864 | 45798 | NSETGGSTNDNT | 3.43 | 1.3 | 0.71 | 1.27 | ATLVT012XX0910 | 45844 | NSQAGGSTNDNA | 0.82 | 2.92 | −0.43 | 1.52 |
| ATLVT012XX0865 | 45799 | NSQAAAASNDNA | 0.66 | 0.86 | 0.26 | −0.65 | ATLVT012XX0911 | 45845 | NSQAGGSTNDNH | 1.39 | 0.95 | 0.41 | 4.39 |
| ATLVT012XX0866 | 45800 | NSQAAAASNDNH | −0.63 | −0.26 | 0.12 | −0.59 | ATTVT012XX0912 | 45846 | NSQAGGSTNDNT | 3.6 | 3.04 | 1.22 | 2.81 |
| ATLVT012XX0867 | 45801 | NSQAAAASNDNT | 0 | 1.12 | −0.19 | 1.95 | ATLVT012XX0913 | 45847 | NSQSAAASNDNA | 0.99 | −1.29 | −0.23 | 1.19 |
| ATLVT012XX0868 | 45802 | NSQAAAATNDNA | 2.22 | −1.27 | −0.73 | −0.82 | ATLVT012XX0914 | 45848 | NSQSAAASNDNH | −0.56 | −0.04 | 0.68 | −0.75 |
| ATLVT012XX0869 | 45803 | NSQAAAATNDNH | 0.87 | −0.57 | 0.07 | 1.2 | ATLVT012XX0915 | 45849 | NSQSAAASNDNT | −0.55 | 1.29 | 0.79 | 0.56 |
| ATLVT012XX0870 | 45804 | NSQAAAATNDNT | 0.4 | −0.28 | −0.89 | −0.77 | ATLVT012XX0916 | 45850 | NSQSAAATNDNA | −0.25 | −1.17 | 0.23 | −0.56 |
| ATLVT012XX0871 | 45805 | NSQAAASSNDNA | −0.45 | 0.57 | 0.38 | 0.79 | ATLVT012XX0917 | 45851 | NSQSAAATNDNH | −1.28 | 0.15 | 0.6 | 0.13 |
| ATLVT012XX0872 | 45806 | NSQAAASSNDNH | −2.68 | −1.32 | 0.39 | −0.75 | ATLVT012XX0918 | 45852 | NSQSAAATNDNT | −0.77 | −1.28 | −0.07 | 0.13 |
| ATLVT012XX0873 | 45807 | NSQAAASSNDNT | −0.89 | 0.98 | 0.36 | −1.57 | ATLVT012XX0919 | 45853 | NSQSAASSNDNA | 0.76 | 0.97 | −0.68 | −0.07 |
| ATLVT012XX0874 | 45808 | NSQAAASTNDNA | −3.24 | −1.67 | −1.08 | −1.72 | ATLVT012XX0920 | 45854 | NSQSAASSNDNH | 0.93 | 0.27 | 0.32 | −1.65 |
| ATLVT012XX0921 | 45855 | NSQSAASSNDNT | −0.46 | 0.08 | −0.54 | −2.2 | ATLVT012XX0967 | 45901 | NSQTAASSNDNA | 1.12 | 1.36 | 0.48 | 0 |
| ATLVT012XX0922 | 45856 | NSQSAASTNDNA | 0.52 | −0.01 | 0.91 | −0.88 | ATLVT012XX0968 | 45902 | NSQTAASSNDNH | 0.17 | 3.76 | −1.24 | −0.44 |
| ATLVT012XX0923 | 45857 | NSQSAASTNDNH | −0.35 | 0.4 | 0.49 | −1.76 | ATLVT012XX0969 | 45903 | NSQTAASSNDNT | −0.03 | 1.74 | −0.96 | −0.66 |
| ATLVT012XX0924 | 45858 | NSQSAASTNDNT | −0.44 | 1.22 | 0.33 | −1.2 | ATLVT012XX0970 | 45904 | NSQTAASTNDNA | −0.48 | −0.01 | 0.96 | 0.96 |
| ATLVT012XX0925 | 45859 | NSQSAGASNDNA | 0.05 | −1.52 | −0.47 | 0.44 | ATLVT012XX0971 | 45905 | NSQTAASTNDNH | 0.72 | 2.56 | −0.25 | 0.13 |
| ATLVT012XX0926 | 45860 | NSQSAGASNDNH | −1.02 | −1.93 | 0.09 | −0.26 | ATLVT012XX0972 | 45906 | NSQTAASTNDNT | −0.37 | 1.64 | 0.23 | 0.68 |
| ATLVT012XX0927 | 45861 | NSQSAGASNDNT | −0.07 | −1.63 | −1.95 | −0.43 | ATLVT012XX0973 | 45907 | NSQTAGASNDNA | 0.54 | −0.3 | −1.55 | −0.75 |
| ATLVT012XX0928 | 45862 | NSQSAGATNDNA | 0.1 | −1.53 | −0.35 | −0.24 | ATLVT012XX0974 | 45908 | NSQTAGASNDNH | 0.89 | −0.39 | −0.04 | −0.82 |
| ATLVT012XX0929 | 45863 | NSQSAGATNDNH | 0.47 | −0.81 | 0.01 | 2.25 | ATLVT012XX0975 | 45909 | NSQTAGASNDNT | −0.85 | 0.13 | −0.27 | −1.02 |
| ATLVT012XX0930 | 45864 | NSQSAGATNDNT | −1.02 | −1.32 | −1.1 | −0.02 | ATLVT012XX0976 | 45910 | NSQTAGATNDNA | −0.99 | 1.28 | −0.21 | 0.17 |
| ATLVT012XX0931 | 45865 | NSQSAGSSNDNA | −0.03 | 0.16 | −0.59 | 0.01 | ATLVT012XX0977 | 45911 | NSQTAGATNDNH | −0.1 | 0.02 | −0.64 | 2.74 |
| ATLVT012XX0932 | 45866 | NSQSAGSSNDNH | 0.9 | −1.64 | 1.51 | −0.52 | ATLVT012XX0978 | 45912 | NSQTAGATNDNT | −0.48 | −0.29 | 0.24 | 0.22 |
| ATLVT012XX0933 | 45867 | NSQSAGSSNDNT | −0.09 | −1.21 | 0.5 | 0.19 | ATLVT012XX0979 | 45913 | NSQTAGSSNDNA | −3.39 | 2.11 | −0.95 | 0.55 |
| ATLVT012XX0934 | 45868 | NSQSAGSTNDNA | 0.37 | 1.41 | −0.48 | 1.36 | ATLVT012XX0980 | 45914 | NSQTAGSSNDNH | −3.26 | −0.03 | 0.2 | 0.85 |
| ATLVT012XX0935 | 45869 | NSQSAGSTNDNH | 0.89 | 1.97 | 1.42 | 2.0 | ATLVT012XX0981 | 45915 | NSQTAGSSNDNT | −2.44 | 1.51 | −0.95 | 0.13 |
| ATLVT012XX0936 | 45870 | NSQSAGSTNDNT | 0.65 | −0.44 | −0.86 | 1.45 | ATLVT012XX0982 | 45916 | NSQTAGSTNDNA | −1.81 | 1.18 | −0.41 | 0.46 |
| ATLVT012XX0937 | 45871 | NSQSGAASNDNA | −2.31 | 1.43 | −0.7 | −1.82 | ATLVT012XX0983 | 45917 | NSQTAGSTNDNH | −2.3 | 0.58 | 0.56 | 0.93 |
| ATLVT012XX0938 | 45872 | NSQSGAASNDNH | −2 | 0.98 | −0.65 | 2.16 | ATLVT012XX0984 | 45918 | NSQTAGSTNDNT | −0.78 | 2.58 | 0.09 | −0.02 |
| ATLVT012XX0939 | 45873 | NSQSGAASNDNT | −1.65 | 2.19 | −2.16 | −2.76 | ATLVT012XX0985 | 45919 | NSQTGAASNDNA | −3.5 | −1.72 | −2.15 | −1.28 |
| ATLVT012XX0940 | 45874 | NSQSGAATNDNA | −1.16 | 0.9 | −0.79 | −1.45 | ATLVT012XX0986 | 45920 | NSQTGAASNDNH | −2.64 | −0.6 | −1.12 | −1.38 |
| ATLVT012XX0941 | 45875 | NSQSGAATNDNH | 0.8 | −0.86 | −1.1 | 0.41 | ATLVT012XX0987 | 45921 | NSQTGAASNDNT | −3.52 | 1.33 | −1.75 | −1.54 |
| ATLVT012XX0942 | 45876 | NSQSGAATNDNT | −1.63 | −1.29 | −0.34 | −0.29 | ATLVT012XX0988 | 45922 | NSQTGAATNDNA | 0.01 | −0.78 | −0.78 | −0.3 |
| ATLVT012XX0943 | 45877 | NSQSGASSNDNA | −1.63 | 0.7 | −0.24 | −0.87 | ATLVT012XX0989 | 45923 | NSQTGAATNDNH | 0.28 | −1.06 | −1.29 | 0.51 |
| ATLVT012XX0944 | 45878 | NSQSGASSNDNH | −0.91 | 2.59 | −0.86 | −0.77 | ATLVT012XX0990 | 45924 | NSQTGAATNDNT | −1.78 | −2.73 | −0.53 | −1.03 |
| ATLVT012XX0945 | 45879 | NSQSGASSNDNT | −0.86 | 1.97 | 0.54 | 0 | ATLVT012XX0991 | 45925 | NSQTGASSNDNA | −1.39 | −0.23 | 0.23 | 0.96 |
| ATLVT012XX0946 | 45880 | NSQSGASTNDNA | 0.36 | −0.93 | 0.68 | 0.64 | ATLVT012XX0992 | 45926 | NSQTGASSNDNH | −1.34 | −1.27 | 1.02 | −0.31 |
| ATLVT012XX0947 | 45881 | NSQSGASTNDNH | 2.88 | 0.13 | 0.35 | 0.71 | ATLVT012XX0993 | 45927 | NSQTGASSNDNT | −1.59 | −0.55 | −0.28 | 0.06 |
| ATLVT012XX0948 | 45882 | NSQSGASTNDNT | 3.81 | −0.34 | 1.07 | −1.44 | ATLVT012XX0994 | 45928 | NSQTGASTNDNA | −2.24 | −0.22 | 0.57 | −0.26 |
| ATLVT012XX0949 | 45883 | NSQSGGASNDNA | 1.68 | 1.36 | 0.52 | 0.06 | ATLVT012XX0995 | 45929 | NSQTGASTNDNH | −0.48 | −1.78 | −0.52 | −0.42 |
| ATLVT012XX0950 | 45884 | NSQSGGASNDNH | 3.77 | 2.36 | 0.25 | 1.75 | ATLVT012XX0996 | 45930 | NSQTGASTNDNT | −0.92 | −1.62 | 1.48 | −1.44 |
| ATLVT012XX0951 | 45885 | NSQSGGASNDNT | 2.49 | 5.23 | 0.51 | 2.3 | ATLVT012XX0997 | 45931 | NSQTGGASNDNA | 0 | 4.04 | 0.67 | 1.7 |
| ATLVT012XX0952 | 45886 | NSQSGGATNDNA | 2.05 | 1.85 | 0.91 | 2.61 | ATLVT012XX0998 | 45932 | NSQTGGASNDNH | 2.81 | 4.31 | 0.87 | 0.19 |
| ATLVT012XX0953 | 45887 | NSQSGGATNDNH | 4.87 | 3.77 | 1.1 | 3.34 | ATLVT012XX0999 | 45933 | NSQTGGASNDNT | 2.13 | 2.49 | 0.32 | 1.9 |
| ATLVT012XX0954 | 45888 | NSQSGGATNDNT | 3.06 | 2.41 | 0.74 | 2.63 | ATLVT012XX1000 | 45934 | NSQTGGATNDNA | 2.44 | −0.55 | 0.81 | 3.34 |
| ATLVT012XX0955 | 45889 | NSQSGGSSNDNA | 1.67 | 2.26 | −0.34 | 2.1 | ATLVT012XX1001 | 45935 | NSQTGGATNDNH | 3.84 | 3.69 | 1.27 | 3.8 |
| ATLVT012XX0956 | 45890 | NSQSGGSSNDNH | 0.54 | 1.71 | −0.2 | 2.37 | ATLVT012XX1002 | 45936 | NSQTGGATNDNT | 1.35 | 2.58 | 0.77 | 1.9 |
| ATLVT012XX0957 | 45891 | NSQSGGSSNDNT | 3.27 | 4.37 | 0.29 | 3.23 | ATLVT012XX1003 | 45937 | NSQTGGSSNDNA | 1.29 | 2.6 | −0.11 | 1.44 |
| ATLVT012XX0958 | 45892 | NSQSGGSTNDNA | 2.69 | 4.32 | 0.64 | 2.92 | ATLVT012XX1004 | 45938 | NSQTGGSSNDNH | 0.53 | 2.64 | 1.69 | 0.87 |
| ATLVT012XX0959 | 45893 | NSQSGGSTNDNH | 3.33 | 2.8 | 1.03 | 2.65 | ATLVT012XX1005 | 45939 | NSQTGGSSNDNT | 1.63 | 1.88 | 1.02 | 2.65 |
| ATLVT012XX0960 | 45894 | NSQSGGSTNDNT | 5.76 | 5.19 | 1.53 | 3.49 | ATLVT012XX1006 | 45940 | NSQTGGSTNDNA | 2.39 | 2.33 | 1.26 | 1.34 |
| ATLVT012XX0961 | 45895 | NSQTAAASNDNA | −1.88 | −1.22 | 1.25 | −0.78 | ATLVT012XX1007 | 45941 | NSQTGGSTNDNH | 1.77 | 1.03 | 0.5 | 2.53 |
| ATLVT012XX0962 | 45896 | NSQTAAASNDNH | −2.4 | 1.97 | 0.51 | −0.12 | ATLVT012XX1008 | 45942 | NSQTGGSTNDNT | 4.53 | 3.04 | 1.03 | 3.08 |
| ATLVT012XX0963 | 45897 | NSQTAAASNDNT | −0.77 | 0.43 | −0.3 | −0.05 | ATLVT012XX1009 | 45943 | NSTAAAASNDNA | 0 | 0.55 | 0.6 | 0.31 |
| ATLVT012XX0964 | 45898 | NSQTAAATNDNA | 1.12 | −0.48 | 2.31 | 0.87 | ATLVT012XX1010 | 45944 | NSTAAAASNDNH | 0.95 | −0.17 | 0.43 | 0.15 |
| ATLVT012XX0965 | 45899 | NSQTAAATNDNH | −0.83 | −0.91 | 1.07 | 3.1 | ATLVT012XX1011 | 45945 | NSTAAAASNDNT | 0.84 | 2.15 | 0.11 | −0.08 |
| ATLVT012XX0966 | 45900 | NSQTAAATNDNT | 0.88 | −0.05 | 0.88 | 1.88 | ATLVT012XX1012 | 45946 | NSTAAAATNDNA | 1.68 | 0.13 | −0.04 | 1.33 |
| ATLVT012XX1013 | 45947 | NSTAAAATNDNH | 2.02 | 1.49 | 0.04 | 1.68 | ATLVT012XX1059 | 45993 | NSTSAAASNDNT | 1.68 | 4.51 | 0.17 | −1.03 |
| ATLVT012XX1014 | 45948 | NSTAAAATNDNT | 1.29 | 1.48 | −0.43 | 0.45 | ATLVT012XX1060 | 45994 | NSTSAAATNDNA | 0.87 | 2.03 | 0.2 | 0.45 |
| ATLVT012XX1015 | 45949 | NSTAAASSNDNA | 0.68 | 0.81 | 0.04 | −0.33 | ATLVT012XX1061 | 45995 | NSTSAAATNDNH | 2.37 | 1.68 | −0.12 | 1.34 |
| ATLVT012XX1016 | 45950 | NSTAAASSNDNH | −0.73 | 1.37 | −0.27 | 1.6 | ATLVT012XX1062 | 45996 | NSTSAAATNDNT | 1.95 | 0.73 | 0.53 | 1.34 |
| ATLVT012XX1017 | 45951 | NSTAAASSNQNT | 1.75 | 0.02 | −0.42 | 0.15 | ATLVT012XX1063 | 45997 | NSTSAASSNDNA | 3.97 | 1.12 | −0.64 | −2.7 |
| ATLVT012XX1018 | 45952 | NSTAAASTNDNA | 2.01 | 1.65 | 1.25 | 1.22 | ATLVT012XX1064 | 45998 | NSTSAASSNDNH | 2.34 | 1.37 | −1.38 | 0.13 |
| ATLVT012XX1019 | 45953 | NSTAAASTNDNH | 0.91 | 0.46 | −0.16 | −0.11 | ATLVT012XX1065 | 45999 | NSTSAASSNDNT | 1.37 | 1.44 | 0.42 | −1.78 |
| ATLVT012XX1020 | 45954 | NSTAAASTNDNT | 1.03 | 2.03 | −0.4 | −0.72 | ATLVT012XX1066 | 46000 | NSTSAASTNDNA | 2.05 | 0.65 | −0.06 | 1.36 |
| ATLVT012XX1021 | 45955 | NSTAAGASNDNA | 2.79 | 2.07 | −1.16 | 0.56 | ATLVT012XX1067 | 46001 | NSTSAASTNDNH | 2.07 | 0.76 | 0.58 | −1.21 |
| ATLVT012XX1022 | 45956 | NSTAAGASNDNH | 2.21 | 2.02 | −0.61 | 1.35 | ATLVT012XX1068 | 46002 | NSTSAASTNDNT | 0.86 | 0.87 | −0.01 | 0.13 |
| ATLVT012XX1023 | 45957 | NSTAAGASNDNT | 3.64 | 2.93 | 0.15 | 1.94 | ATLVT012XX1069 | 46003 | NSTSAGASNDNA | 2.2 | 5.41 | 0.14 | 3.65 |
| ATLVT012XX1024 | 45958 | NSTAAGATNDNA | 0.93 | 2.91 | 0.66 | 0.8 | ATLVT012XX1070 | 46004 | NSTSAGASNDNH | 2.34 | 3.93 | 0.95 | 2.03 |
| ATLVT012XX1025 | 45959 | NSTAAGATNDNH | 2.68 | 5.8 | 0.52 | 1.21 | ATLVT012XX1071 | 46005 | NSTSAGASNQNT | 3.68 | 4.43 | 0.63 | 4.22 |
| ATLVT012XX1026 | 45960 | NSTAAGATNDNT | 2.13 | 2.63 | 0.49 | 0.65 | ATLVT012XX1072 | 46006 | NSTSAGATNDNA | 2.62 | 2.76 | −0.47 | 2.03 |
| ATLVT012XX1027 | 45961 | NSTAAGSSNDNA | 3.49 | 0.51 | −0.19 | 0.75 | ATLVT012XX1073 | 46007 | NSTSAGATNDNH | 4.47 | 3.84 | −0.56 | 2.42 |
| ATLVT012XX1028 | 45962 | NSTAAGSSNDNH | 1.92 | 0.35 | −0.88 | −0.19 | ATLVT012XX1074 | 46008 | NSTSAGATNDNT | 5.03 | 4.81 | −0.07 | 3.25 |
| ATLVT012XX1029 | 45963 | NSTAAGSSNDNT | 3.9 | 1.96 | 0.41 | 2.69 | ATLVT012XX1075 | 46009 | NSTSAGSSNDNA | 2.35 | 3.35 | −0.13 | 2.33 |
| ATLVT012XX1030 | 45964 | NSTAAGSTNDNA | 3.24 | 5.19 | 0.1 | 2.09 | ATLVT012XX1076 | 46010 | NSTSAGSSNDNH | 3.06 | 2.94 | −0.32 | 1.49 |
| ATLVT012XX1031 | 45965 | NSTAAGSTNDNH | 2.62 | 2.18 | 0.8 | 0.94 | ATLVT012XX1077 | 46011 | NSTSAGSSNDNT | 5.38 | 6.49 | 1.29 | 3.02 |
| ATLVT012XX1032 | 45966 | NSTAAGSTNDNT | 4.38 | 5.05 | 1.02 | 3.04 | ATLVT012XX1078 | 46012 | NSTSAGSTNDNA | 3.08 | 2.33 | 1.52 | 2.4 |
| ATLVT012XX1033 | 45967 | NSTAGAASNDNA | 1.26 | 2.25 | −0.9 | −0.23 | ATLVT012XX1079 | 46013 | NSTSAGSTNDNH | 2.81 | 3.21 | −0.13 | 2.47 |
| ATLVT012XX1034 | 45968 | NSTAGAASNDNH | 1.69 | 1.03 | −2.58 | −0.58 | ATLVT012XX1080 | 46014 | NSTSAGSTNDNT | 5.67 | 6.94 | 2.01 | 4.29 |
| ATLVT012XX1035 | 45969 | NSTAGAASNDNT | 3.38 | 1.05 | −2.25 | −0.77 | ATLVT012XX1081 | 46015 | NSTSGAASNDNA | 3.18 | 2.94 | −1.78 | 1.6 |
| ATLVT012XX1036 | 45970 | NSTAGAATNDNA | 2.27 | 3.46 | −1.81 | 0 | ATLVT012XX1082 | 46016 | NSTSGAASNDNH | 3.56 | 3.6 | −0.89 | −0.45 |
| ATLVT012XX1037 | 45971 | NSTAGAATNDNH | 1.12 | 3.36 | −3.43 | 1.42 | ATLVT012XX1083 | 46017 | NSTSGAASNDNT | 2.62 | 2.86 | −0.18 | 0.66 |
| ATLVT012XX1038 | 45972 | NSTAGAATNDNT | 0.4 | 4.05 | −2.18 | 0.74 | ATLVT012XX1084 | 46018 | NSTSGAATNDNA | 2.5 | 3.68 | −1.86 | 1.4 |
| ATLVT012XX1039 | 45973 | NSTAGASSNDNA | 1.95 | 4.4 | 0.06 | 1.75 | ATLVT012XX1085 | 46019 | NSTSGAATNDNH | 3.26 | 3.63 | 0.55 | 1.4 |
| ATLVT012XX1040 | 45974 | NSTAGASSNDNH | 2.18 | 2.94 | −0.42 | 0.39 | ATLVT012XX1086 | 46020 | NSTSGAATNDNT | 3.02 | 3.52 | 0.41 | 1.06 |
| ATLVT012XX1041 | 45975 | NSTAGASSNDNT | 2.9 | 3.23 | −0.32 | 1.7 | ATLVT012XX1087 | 46021 | NSTSGASSNDNH | 2.1 | 4.26 | −0.78 | 0 |
| ATLVT012XX1042 | 45976 | NSTAGASTNDNA | 4.3 | 3.61 | 0.9 | 3.47 | ATLVT012XX1088 | 46022 | NSTSGASSNDNT | 3.3 | 3.07 | −0.32 | 1.65 |
| ATLVT012XX1043 | 45977 | NSTAGASTNDNH | 3.03 | 3.39 | 0.1 | 2.5 | ATLVT012XX1089 | 46023 | NSTSGASTNDNH | 3.27 | 3.21 | −0.83 | 1.26 |
| ATLVT012XX1044 | 45978 | NSTAGASTNDNT | 5.63 | 5.23 | −0.09 | 2.95 | ATLVT012XX1090 | 46024 | NSTSGASTNDNT | 5.39 | 5.39 | 0.64 | 2.16 |
| ATLVT012XX1045 | 45979 | NSTAGGASNDNA | 9.46 | 10.07 | 0.8 | 4.69 | ATLVT012XX1091 | 46025 | NSTSGGASNDNA | 9.7 | 12.41 | 1.52 | 5.44 |
| ATLVT012XX1046 | 45980 | NSTAGGASNDNH | 8.12 | 7.8 | 0.84 | 3.51 | ATLVT012XX1092 | 46026 | NSTSGGASNDNH | 7.98 | 9.47 | 1.07 | 3.41 |
| ATLVT012XX1047 | 45981 | NSTAGGASNDNT | 10.86 | 12.93 | 1.72 | 4.49 | ATLVT012XX1093 | 46027 | NSTSGGASNDNT | 11.16 | 13.46 | 1.63 | 3.8 |
| ATLVT012XX1048 | 45982 | NSTAGGATNDNA | 8.53 | 9.79 | 0.52 | 4.79 | ATLVT012XX1094 | 46028 | NSTSGGATNDNA | 8.76 | 11.94 | 1.88 | 5.31 |
| ATLVT012XX1049 | 45983 | NSTAGGATNDNH | 9.34 | 10.59 | 0.73 | 3.34 | ATLVT012XX1095 | 46029 | NSTSGGATNDNH | 8.8 | 10.51 | 1.1 | 4.12 |
| ATLVT012XX1050 | 45984 | NSTAGGATNDNT | 10.38 | 13.68 | 1.19 | 3.1 | ATLVT012XX1096 | 46030 | NSTSGGATNDNT | 11.07 | 13.53 | 1.25 | 4.22 |
| ATLVT012XX1051 | 45985 | NSTAGGSSNDNA | 11.01 | 13.41 | 0.04 | 3.87 | ATLVT012XX1097 | 46031 | NSTSGGSSNDNH | 8.46 | 9.88 | 1.13 | 3.37 |
| ATLVT012XX1052 | 45986 | NSTAGGSSNDNH | 8.87 | 10.24 | 0.31 | 3.37 | ATLVT012XX1098 | 46032 | NSTSGGSSNDNT | 11.01 | 12.21 | 0.03 | 3.25 |
| ATLVT012XX1053 | 45987 | NSTAGGSSNDNT | 11.28 | 12.66 | −0.59 | 2.98 | ATLVT012XX1099 | 46033 | NSTSGGSTNDNA | 11.31 | 13.26 | 1.54 | 4.09 |
| ATLVT012XX1054 | 45988 | NSTAGGSTNDNA | 11.14 | 13.89 | 0.45 | 3.7 | ATLVT012XX1100 | 46034 | NSTSGGSTNDNH | 9.42 | 10.77 | 0.79 | 3.51 |
| ATLVT012XX1055 | 45989 | NSTAGGSTNDNH | 9.45 | 11.15 | −0.11 | 4.56 | ATLVT012XX1101 | 46035 | NSTSGGSTNDNT | 11.18 | 12.48 | 0.3 | 2.68 |
| ATLVT012XX1056 | 45990 | NSTAGGSTNDNT | 11.18 | 11.98 | −0.94 | 2.52 | ATLVT012XX1102 | 46036 | NSTTAAASNDNA | 3.05 | 2.29 | 0.68 | 1.76 |
| ATLVT012XX1057 | 45991 | NSTSAAASNDNA | 0.33 | 1.45 | −0.78 | −0.98 | ATLVT012XX1103 | 46037 | NSTTAAASNDNH | 1.71 | 2.02 | 0.73 | 0.72 |
| ATLVT012XX1058 | 45992 | NSTSAAASNDNH | 1.01 | 1.43 | 0.66 | −0.68 | ATLVT012XX1104 | 46038 | NSTTAAASNDNT | 2.63 | −0.05 | −0.12 | 1.4 |
| ATLVT012XX1105 | 46039 | NSTTAAATNDNA | 2.43 | 6.26 | 0.22 | −0.34 | ATLVT012XX1151 | 46085 | SGAAAAASNDNH | −1.11 | 1.26 | −1.64 | −2.75 |
| ATLVT012XX1106 | 46040 | NSTTAAATNDNH | 2.99 | 3.12 | 0.43 | 0.87 | ATLVT012XX1152 | 46086 | SGAAAAASNDNT | −2.08 | 0.57 | −1.5 | −2.35 |
| ATLVT012XX1107 | 46041 | NSTTAAATNDNT | 1.03 | 3 | 0.68 | 0.22 | ATLVT012XX1153 | 46087 | SGAAAAATNDNA | −0.71 | 2.79 | −1.38 | −0.16 |
| ATLVT012XX1108 | 46042 | NSTTAASSNDNA | 0.59 | −1.24 | −0.11 | 0.58 | ATLVT012XX1154 | 46088 | SGAAAAATNDNH | 0.2 | 1.03 | −1.49 | −0.37 |
| ATLVT012XX1109 | 46043 | NSTTAASSNDNH | 2.05 | 0.49 | 0.89 | −0.15 | ATLVT012XX1155 | 46089 | SGAAAAATNDNT | 1.81 | 0.46 | −0.08 | −1.33 |
| ATLVT012XX1110 | 46044 | NSTTAASSNDNT | 2.43 | −0.66 | 0.25 | 0.45 | ATLVT012XX1156 | 46090 | SGAAAASSNDNA | −3.07 | −0.49 | −0.48 | −1.34 |
| ATLVT012XX1111 | 46045 | NSTTAASTNDNA | 1.96 | 2.4 | −0.65 | −0.15 | ATLVT012XX1157 | 46091 | SGAAAASSNDNH | −2.14 | 2.27 | −0.95 | −1.2 |
| ATLVT012XX1112 | 46046 | NSTTAASTNDNH | 0.5 | 2.09 | 0.52 | −1.17 | ATLVT012XX1158 | 46092 | SGAAAASSNDNT | −1.26 | 0.85 | −0.24 | −1.95 |
| ATLVT012XX1113 | 46047 | NSTTAASTNDNT | 3.02 | 2.37 | −0.57 | 1.05 | ATLVT012XX1159 | 46093 | SGAAAASTNDNA | −2.06 | 0.6 | 0.75 | −1.01 |
| ATLVT012XX1114 | 46048 | NSTTAGASNDNA | 1.26 | 1.89 | 0.96 | 1.47 | ATLVT012XX1160 | 46094 | SGAAAASTNDNH | −2.71 | 1.23 | −0.16 | −1.11 |
| ATLVT012XX1115 | 46049 | NSTTAGASNDNH | 2.68 | 2.38 | 0.18 | 2.67 | ATLVT012XX1161 | 46095 | SGAAAASTNDNT | −1 | 1.42 | −2.27 | −2.42 |
| ATLVT012XX1116 | 46050 | NSTTAGASNDNT | 4.98 | 3 | 1.48 | 3.55 | ATLVT012XX1162 | 46096 | SGAAAGASNDNA | −3.36 | −1.01 | −0.84 | −1.85 |
| ATLVT012XX1117 | 46051 | NSTTAGATNDNA | 0.85 | 2.25 | −0.21 | 1.28 | ATLVT012XX1163 | 46097 | SGAAAGASNDNH | −1 | −0.06 | −0.7 | −1.17 |
| ATLVT012XX1118 | 46052 | NSTTAGATNDNH | 2.13 | 2.85 | 0.65 | 1.88 | ATLVT012XX1164 | 46098 | SGAAAGASNDNT | −2.27 | −1.24 | 0.13 | −2.24 |
| ATLVT012XX1119 | 46053 | NSTTAGANDNT | 1.8 | 2.94 | 0.7 | 2.58 | ATLVT012XX1165 | 46099 | SGAAAGATNDNA | −0.63 | 3.01 | −1.42 | 0.98 |
| ATLVT012XX1120 | 46054 | NSTTAGSSNDNA | 1.37 | 2.42 | 1.18 | 3.41 | ATLVT012XX1166 | 46100 | SGAAAGATNDNH | −0.78 | 1.24 | 1.38 | 1.31 |
| ATLVT012XX1121 | 46055 | NSTTAGSSNDNH | 1.73 | 0.25 | 0.81 | 0.54 | ATLVT012XX1167 | 46101 | SGAAAGATNDNT | 0.09 | 2.08 | −1.46 | 0.94 |
| ATLVT012XX1122 | 46056 | NSTTAGSSNDNT | 3.57 | 2.84 | 0.95 | 2.94 | ATLVT012XX1168 | 46102 | SGAAAGSSNDNA | 0.12 | −2.55 | −0.95 | −1.62 |
| ATLVT012XX1123 | 46057 | NSTTAGSTNDNA | 1.56 | 2.05 | 0.25 | 2.1 | ATLVT012XX1169 | 46103 | SGAAAGSSNDNH | 1.13 | 2.48 | −0.44 | 0.57 |
| ATLVT012XX1124 | 46058 | NSTTAGSTNDNH | 2.06 | 1.76 | −0.09 | 1.58 | ATLVT012XX1170 | 46104 | SGAAAGSSNDNT | −1.73 | −1.09 | −1.49 | −0.66 |
| ATLVT012XX1125 | 46059 | NSTTAGSTNDNT | 3.37 | 5.77 | 1.8 | 3.16 | ATLVT012XX1171 | 46105 | SGAAAGSTNDNA | −1.5 | 2.01 | −0.26 | 0.2 |
| ATLVT012XX1126 | 46060 | NSTTGAASNDNA | 1.78 | 1.21 | −0.03 | −1.35 | ATLVT012XX1172 | 46106 | SGAAAGSTNDNH | −2.56 | 1.11 | 0.16 | 2.44 |
| ATLVT012XX1127 | 46061 | NSTTGAASNDNH | 1.26 | 2.53 | −15 | −1.52 | ATLVT012XX1173 | 46107 | SGAAAGSTNDNT | −1.14 | 0.52 | 0.55 | 1.71 |
| ATLVT012XX1128 | 46062 | NSTTGAASNDNT | 1.48 | 2.92 | −0.98 | −1.79 | ATLVT012XX1174 | 46108 | SGAAGAASNDNA | −1.62 | −0.67 | −2.67 | −2.7 |
| ATLVT012XX1129 | 46063 | NSTTGAATNDNA | 1.5 | 3.25 | −1.77 | 0.14 | ATLVT012XX1175 | 46109 | SGAAGAASNDNH | 1.63 | 1.14 | 2.27 | 0.83 |
| ATLVT012XX1130 | 46064 | NSTTGAATNDNH | 1.69 | 2.64 | −1.74 | 1.91 | ATLVT012XX1176 | 46110 | SGAAGAASNDNT | −1.77 | −2.09 | −2.17 | −1.3 |
| ATLVT012XX1131 | 46065 | NSTTGAATNDNT | 1.62 | 1.25 | 0.13 | −0.27 | ATLVT012XX1177 | 46111 | SGAAGAATNDNA | 0.78 | −0.79 | −4.35 | 0.18 |
| ATLVT012XX1132 | 46066 | NSTTGASSNDNA | 2.38 | 2.82 | 0.2 | 1.17 | ATLVT012XX1178 | 46112 | SGAAGAATNDNH | 1.77 | 2.14 | −1.71 | 0.94 |
| ATLVT012XX1133 | 46067 | NSTTGASSNDNH | 2.93 | 1.86 | −0.34 | 0.05 | ATLVT012XX1179 | 46113 | SGAAGAATNDNT | 1.58 | 1.67 | −2.87 | −0.81 |
| ATLVT012XX1134 | 46068 | NSTTGASSNDNT | 3.45 | 1.89 | 0.66 | −1.11 | ATLVT012XX1180 | 46114 | SGAAGASSNDNA | −1.8 | 2.41 | 0.22 | −3.56 |
| ATLVT012XX1135 | 46069 | NSTTGASTNDNA | 2.79 | 1.07 | 1.16 | 0.68 | ATLVT012XX1181 | 46115 | SGAAGASSNDNH | −0.52 | 0.79 | −1.08 | −2.25 |
| ATLVT012XX1136 | 46070 | NSTTGASTNDNH | 1.84 | 1.92 | −0.32 | 0.63 | ATLVT012XX1182 | 46116 | SGAAGASSNDNT | −0.55 | 0.58 | −1.19 | −2.67 |
| ATLVT012XX1137 | 46071 | NSTTGASTNDNT | 1.7 | 2.7 | 0.82 | 1.53 | ATLVT012XX1183 | 46117 | SGAAGASTNDNA | −0.87 | 0.42 | 0.19 | 1.44 |
| ATLVT012XX1138 | 46072 | NSTTGGASNDNA | 6.72 | 8.93 | 2.3 | 4.29 | ATLVT012XX1184 | 46118 | SGAAGASTNDNH | 0.32 | 1.31 | 0.11 | −0.53 |
| ATLVT012XX1139 | 46073 | NSTTGGASNDNH | 5.29 | 6.39 | 0.68 | 3.29 | ATLVT012XX1185 | 46119 | SGAAGASTNDNT | −0.15 | 0.32 | −1.27 | −0.66 |
| ATLVT012XX1140 | 46074 | NSTTGGASNDNT | 8.98 | 11.19 | 1.74 | 4.65 | ATLVT012XX1186 | 46120 | SGAAGGASNDNA | 3.94 | 3.94 | −0.53 | 2.79 |
| ATLVT012XX1141 | 46075 | NSTTGGATNDNA | 6.76 | 8.12 | 1.6 | 5.37 | ATLVT012XX1187 | 46121 | SGAAGGASNDNH | 7.1 | 4.45 | 0.79 | 3.97 |
| ATLVT012XX1142 | 46076 | NSTTGGATNDNH | 7.26 | 8.16 | 0.93 | 4.25 | ATLVT012XX1188 | 46122 | SGAAGGASNDNT | 4.86 | 4.79 | −0.57 | 0.5 |
| ATLVT012XX1143 | 46077 | NSTTGGATNDNT | 8.78 | 10.8 | 1.66 | 4.75 | ATLVT012XX1189 | 46123 | SGAAGGATNDNA | 5.1 | 7.93 | −1 | 0.88 |
| ATLVT012XX1144 | 46078 | NSTTGGSSNDNA | 9.2 | 10.34 | 2.23 | 4.68 | ATLVT012XX1190 | 46124 | SGAAGGATNDNH | 9.36 | 11.17 | −2.08 | 2.19 |
| ATLVT012XX1145 | 46079 | NSTTGGSSNDNH | 7.06 | 7.38 | 0.79 | 3.51 | ATLVT012XX1191 | 46125 | SGAAGGATNDNT | 5.15 | 6.51 | −0.75 | 0.17 |
| ATLVT012XX1146 | 46080 | NSTTGGSSNDNT | 8.83 | 11.88 | 0.65 | 3.79 | ATLVT012XX1192 | 46126 | SGAAGGSSNDNA | 1.58 | 3.42 | −0.79 | −0.52 |
| ATLVT012XX1147 | 46081 | NSTTGGSTNDNA | 10.05 | 11.99 | 1.82 | 4.71 | ATLVT012XX1193 | 46127 | SGAAGGSSNDNH | 2.11 | 1.9 | −1.07 | 0.54 |
| ATLVT012XX1148 | 46082 | NSTTGGSTNDNH | 8.27 | 8.69 | 0.68 | 3.72 | ATLVT012XX1194 | 46128 | SGAAGGSSNDNT | 2.69 | 2.37 | −0.1 | 0.09 |
| ATLVT012XX1149 | 46083 | NSTTGGSTNDNT | 10.94 | 12.44 | 0.99 | 3.16 | ATLVT012XX1195 | 46129 | SGAAGGSTNDNA | 5.1 | 4.66 | −0.91 | 1.7 |
| ATLVT012XX1150 | 46084 | SGAAAAASNDNA | −2.1 | 4.68 | −1.33 | −1.14 | ATLVT012XX1196 | 46130 | SGAAGGSTNDNH | 3.77 | 6.29 | −0.02 | 1.61 |
| ATLVT012XX1197 | 46131 | SGAAGGSTNDNT | 4.16 | 6.51 | −0.44 | 2.05 | ATLVT012XX1243 | 46177 | SGASGGSTNDNA | 3.76 | 3.46 | 1.32 | 3.19 |
| ATLVT012XX1198 | 46132 | SGASAAASNDNA | −2.76 | 3.82 | −0.7 | 0.49 | ATLVT012XX1244 | 46178 | SGASGGSTNDNH | 5.81 | 4.21 | 1.17 | 4.18 |
| ATLVT012XX1199 | 46133 | SGASAAASNDNH | −1.67 | 0.41 | −0.71 | 0.16 | ATLVT012XX1245 | 46179 | SGASGGSTNDNT | 3.86 | 2.62 | 1.13 | 2.35 |
| ATLVT012XX1200 | 46134 | SGASAAASNDNT | −1.84 | 0.76 | 0.9 | 1.89 | ATLVT012XX1246 | 46180 | SGATAAASNDNA | −0.21 | 0.38 | 0.4 | −0.78 |
| ATLVT012XX1201 | 46135 | SGASAAATNDNA | −0.68 | −0.28 | −0.24 | 0.81 | ATLVT012XX1247 | 46181 | SGATAAASNDNH | −0.01 | 2.12 | 1.59 | 0.64 |
| ATLVT012XX1202 | 46136 | SGASAAATNDNH | −0.69 | 1.91 | 0.84 | 1.97 | ATLVT012XX1248 | 46182 | SGATAAASNDNT | −1.94 | 2.04 | −0.35 | −0.62 |
| ATLVT012XX1203 | 46137 | SGASAAATNDNT | 0.55 | 0.9 | 0.25 | 0.94 | ATLVT012XX1249 | 46183 | SGATAAATNDNA | 2.88 | −0.38 | 0.35 | −0.41 |
| ATLVT012XX1204 | 46138 | SGASAASSNDNA | −0.43 | −0.98 | −0.63 | −0.57 | ATLVT012XX1250 | 46184 | SGATAAATNDNH | 0.63 | 2.4 | −0.13 | 2.16 |
| ATLVT012XX1205 | 46139 | SGASAASSNDNH | −1.81 | −1.95 | −0.14 | −1.71 | ATLVT012XX1251 | 46185 | SGATAAATNDNT | 1.35 | 0.08 | −0.41 | −0.84 |
| ATLVT012XX1206 | 46140 | SGASAASSNDNT | −0.97 | −2.23 | −1.03 | −0.7 | ATLVT012XX1252 | 46186 | SGATAASSNDNA | −0.61 | 0.71 | 1.19 | 0.6 |
| ATLVT012XX1207 | 46141 | SGASAASTNDNA | 1.02 | 1.62 | 1.6 | 0.8 | ATLVT012XX1253 | 46187 | SGATAASSNDNH | −0.69 | 0.8 | 0.01 | 0.64 |
| ATLVT012XX1208 | 46142 | SGASAASTNDNH | 2.76 | 0.77 | 0.46 | 0.07 | ATLVT012XX1254 | 46188 | SGATAASSNDNT | −0.63 | −0.21 | 1.09 | −0.59 |
| ATLVT012XX1209 | 46143 | SGASAASTNQNT | 3.75 | 0.7 | −0.67 | 0.71 | ATLVT012XX1255 | 46189 | SGATAASTNDNA | −0.74 | −0.05 | 1 | 0.25 |
| ATLVT012XX1210 | 46144 | SGASAGASNDNA | −0.26 | 0.84 | −0.89 | 0.37 | ATLVT012XX1256 | 46190 | SGATAASTNDNH | −1.08 | 0.76 | 0.63 | −0.03 |
| ATLVT012XX1211 | 46145 | SGASAGASNDNH | −0.99 | 2.97 | 1.02 | 1.2 | ATLVT012XX1257 | 46191 | SGATAASTNDNT | −1.07 | −0.78 | −0.53 | 0.57 |
| ATLVT012XX1212 | 46146 | SGASAGASNDNT | 0.47 | −0.27 | −0.12 | −0.13 | ATLVT012XX1258 | 46192 | SGATAGASNDNA | −0.82 | 1.3 | −0.14 | −0.22 |
| ATLVT012XX1213 | 46147 | SGASAGATNDNA | −0.16 | 3.73 | 1.22 | 1.04 | ATTVT012XX1259 | 46193 | SGATAGASNDNH | −0.62 | 1.1 | 0.85 | 1.9 |
| ATLVT012XX1214 | 46148 | SGASAGATNDNH | 1.24 | 2.88 | 1.43 | 2.19 | ATLVT012XX1260 | 46194 | SGATAGASNDNT | −0.45 | 2.17 | 0.09 | 1.69 |
| ATLVT012XX1215 | 46149 | SGASAGATNDNT | 2.68 | 3.34 | 0.71 | 0.24 | ATLVT012XX1261 | 46195 | SGATAGATNDNA | −0.75 | 2.02 | −0.44 | 0.46 |
| ATLVT012XX1216 | 46150 | SGASAGSSNDNA | −0.42 | 0.89 | 0.51 | −0.43 | ATLVT012XX1262 | 46196 | SGATAGATNDNH | 1.35 | 1.51 | 0.8 | 3.5 |
| ATLVT012XX1217 | 46151 | SGASAGSSNDNH | 0.57 | 2.84 | 1.09 | 0.64 | ATLVT012XX1263 | 46197 | SGATAGATNDNT | 3.84 | 0.14 | −0.38 | 2.23 |
| ATLVT012XX1218 | 46152 | SGASAGSSNDNT | 0.16 | 3.14 | −0.26 | −1.4 | ATLVT012XX1264 | 46198 | SGATAGSSNDNA | −2.61 | −1.95 | 0.38 | −0.38 |
| ATLVT01XXX1219 | 46153 | SGASAGSTNDNA | −1.54 | 2.02 | 0.38 | 0.31 | ATLVT012XX1265 | 46199 | SGATAGSSNDNH | −2.25 | 0.07 | −0.38 | 1 |
| ATLVT012XX1220 | 46154 | SGASAGSTNDNH | −0.82 | 1.18 | 1.03 | 2.14 | ATLVT012XX1266 | 46200 | SGATAGSSNDNT | −1.45 | 0.38 | 0.59 | −0.75 |
| ATLVT012XX1221 | 46155 | SGASAGSTNDNT | −1.35 | 0.93 | 0.68 | 1.62 | ATLVT012XX1267 | 46201 | SGATAGSTNDNA | 0.34 | −0.04 | −0.36 | 0.31 |
| ATLVT012XX1222 | 46156 | SGASGAASNDNA | 0.21 | −0.68 | −0.39 | −0.93 | ATLVT012XX1268 | 46202 | SGATAGSTNDNH | −0.08 | −1.08 | −0.36 | 0.67 |
| ATLVT012XX1223 | 46157 | SGASGAASNDNH | 0.58 | 1.27 | 0.52 | −0.05 | ATLVT012XX1269 | 46203 | SGATAGSTNDNT | 1.13 | −0.76 | 0.43 | 1.67 |
| ATLVT012XX1224 | 46158 | SGASGAASNDNT | 0.1 | −0.07 | −0.34 | −0.18 | ATLVT012XX1270 | 46204 | SGATGAASNDNA | 1.91 | −1.61 | −0.18 | −1.32 |
| ATLVT012XX1225 | 46159 | SGASGAATNDNA | 1.21 | 2 | 0.08 | 0.05 | ATLVT012XX1271 | 46205 | SGATGAASNDNH | 0.44 | 0.73 | −1 | 2.37 |
| ATLVT012XX1226 | 46160 | SGASGAATNDNH | 1.29 | 3.83 | −0.32 | 3.91 | ATTVT012XX1272 | 46206 | SGATGAASNDNT | −0.44 | −1.43 | −0.25 | 0.1 |
| ATLVT012XX1227 | 46161 | SGASGAATNDNT | 1.12 | 1.15 | 1.14 | −2.01 | ATLVT012XX1273 | 46207 | SGATGAATNDNA | 0.13 | 1.45 | −1.61 | 0.26 |
| ATLVT012XX1228 | 46162 | SGASGASSNDNA | −0.23 | 1.02 | 1.56 | 0.86 | ATLVT012XX1274 | 46208 | SGATGAATNDNH | 2.54 | 5.14 | 1.7 | 2.11 |
| ATLVT012XX1229 | 46163 | SGASGASSNDNH | 0.25 | 0.43 | 1.69 | 1.63 | ATLVT012XX1275 | 46209 | SGATGAATNDNT | 1.09 | 2.36 | 0 | −0.51 |
| ATLVT012XX1230 | 46164 | SGASGASSNDNT | 0.48 | −0.47 | 0.57 | −0.18 | ATLVT012XX1276 | 46210 | SGATGASSNDNA | −0.31 | 1.34 | 1.01 | −0.51 |
| ATLVT012XX1231 | 46165 | SGASGASTNDNA | −0.11 | −0.76 | 0.83 | 1.84 | ATLVT012XX1277 | 46211 | SGATGASSNDNH | 0.23 | 0.1 | 1.33 | −0.27 |
| ATLVT012XX1232 | 46166 | SGASGASTNDNH | 0.24 | 0.28 | −0.33 | 2.35 | ATLVT012XX1278 | 46212 | SGATGASSNDNT | 0.22 | 0.99 | 1.37 | −0.19 |
| ATLVT012XX1233 | 46167 | SGASGASTNDNT | 0.13 | 0.02 | 1.01 | 1.67 | ATLVT012XX1279 | 46213 | SGATGASTNDNA | 2.91 | 3.44 | 0.91 | −0.52 |
| ATLVT012XX1234 | 46168 | SGASGGASNDNA | 2.47 | 3.05 | 0.6 | 1.38 | ATLVT012XX1280 | 46214 | SGATGASTNDNH | 1.58 | 0.87 | 0.22 | 2.06 |
| ATLVT012XX1235 | 46169 | SGASGGASNDNH | 6.92 | 8.34 | 0.88 | 4.23 | ATLVT012XX1281 | 46215 | SGATGASTNDNT | 2.78 | 0.33 | 1.05 | −0.88 |
| ATLVT012XX1236 | 46170 | SGASGGASNDNT | 3.57 | 5.29 | 0.42 | 1.15 | ATLVT012XX1282 | 46216 | SGATGGASNDNA | 1.19 | 1.2 | −0.39 | 1.82 |
| ATLVT012XX1237 | 46171 | SGASGGATNDNA | 4.02 | 3.91 | 0.87 | 2.25 | ATLVT012XX1283 | 46217 | SGATGGASNDNH | 2.49 | 4.54 | 0.11 | 3.28 |
| ATLVT012XX1238 | 46172 | SGASGGATNDNH | 9.39 | 11.76 | 0.9 | 4.76 | ATLVT012XX1284 | 46218 | SGATGGASNDNT | 2.01 | 2.03 | 1.24 | 1.19 |
| ATLVT012XX1239 | 46173 | SGASGGATNDNT | 4.65 | 4.59 | 1.02 | 2.79 | ATLVT012XX1285 | 46219 | SGATGGATNDNA | 3.94 | 2.84 | 1.68 | 2.76 |
| ATLVT012XX1240 | 46174 | SGASGGSSNDNA | 2.85 | 2.39 | −0.56 | 1.43 | ATLVT012XX1286 | 46220 | SGATGGATNDNH | 8.55 | 9.86 | 1.54 | 4.86 |
| ATLVT012XX1241 | 46175 | SGASGGSSNDNH | 2.26 | 2.39 | 0.22 | 2.74 | ATLVT012XX1287 | 46221 | SGATGGATNDNT | 4 | 3.62 | 1.28 | 1.42 |
| ATLVT012XX1242 | 46176 | SGASGGSSNDNT | 4.09 | 3.65 | 0.81 | 2.59 | ATLVT012XX1288 | 46222 | SGATGGSSNDNA | 1.41 | 1.62 | 0.34 | 1.26 |
| ATLVT012XX1289 | 46223 | SGATGGSSNDNH | 1.5 | 2.07 | 0.01 | 0.88 | ATLVT012XX1335 | 46269 | SGEAGGATNDNT | 5.63 | 6.15 | 1.1 | 2.34 |
| ATLVT01XXX1290 | 46224 | SGATGGSSNDNT | 3.78 | 1.22 | 0.09 | 1.26 | ATLVT012XX1336 | 46270 | SGEAGGSSNDNA | 2.17 | 4.62 | −0.35 | 1.95 |
| ATLVT012XX1291 | 46225 | SGATGGSTNDNA | 3.24 | 2.56 | −0.82 | 3.18 | ATLVT012XX1337 | 46271 | SGEAGGSSNDNH | 2 | 1.34 | 0.02 | 1.95 |
| ATLVT012XX1292 | 46226 | SGATGGSTNDNH | 4.21 | 3.85 | 0.14 | 4.24 | ATLVT012XX1338 | 46272 | SGEAGGSSNDNT | 2.24 | 3.16 | −0.03 | 2.72 |
| ATLVT012XX1293 | 46227 | SGATGGSTNDNT | 2.43 | 2.15 | 0.37 | 2.09 | ATLVT012XX1339 | 46273 | SGEAGGSTNDNA | 3.24 | 4.26 | 0.15 | 1.05 |
| ATLVT012XX1294 | 46228 | SGEAAAASNDNA | −1.17 | 0.79 | −0.68 | 0.89 | ATLVT012XX1340 | 46274 | SGEAGGSTNDNH | 5.25 | 6.05 | 1.44 | 3.54 |
| ATLVT012XX1295 | 46229 | SGEAAAASNDNH | −0.16 | 2.46 | 0.28 | 1.53 | ATLVT012XX1341 | 46275 | SGEAGGSTNDNT | 3.66 | 3.48 | 0.56 | 3.16 |
| ATLVT012XX1296 | 46230 | SGEAAAASNDNT | −1.53 | 2.82 | −0.02 | 1.28 | ATLVT012XX1342 | 46276 | SGESAAASNDNA | −1.37 | −1.44 | −1.16 | 0.38 |
| ATLVT012XX1297 | 46231 | SGEAAAATNDNA | 1.78 | 1.32 | −0.34 | 0.76 | ATLVT012XX1343 | 46277 | SGESAAASNDNH | −0.98 | −1.87 | −0.43 | −1.34 |
| ATLVT01XXX1298 | 46232 | SGEAAAATNDNH | −1.16 | 2.66 | −0.3 | 0.29 | ATLVT012XX1344 | 46278 | SGESAAASNDNT | −2.13 | −2.76 | 0.46 | −0.72 |
| ATLVT012XX1299 | 46233 | SGEAAAATNDNT | 0.45 | 4.07 | −0.88 | −2.05 | ATLVT012XX1345 | 46279 | SGESAAATNDNA | −0.44 | −0.89 | 0.21 | −0.84 |
| ATLVT012XX1300 | 46234 | SGEAAASSNDNA | 0.94 | −1.01 | −0.2 | −2.09 | ATLVT012XX1346 | 46280 | SGESAAATNDNH | 0.34 | 0.09 | −1.11 | −0.68 |
| ATLVT012XX1301 | 46235 | SGEAAASSNDNH | 0.9 | −1.15 | 0.28 | −1.14 | ATLVT012XX1347 | 46281 | SGESAAATNDNT | 0 | −1.49 | −0.01 | −0.47 |
| ATLVT012XX1302 | 46236 | SGEAAASSNDNT | 0.25 | 0.06 | 0.67 | −1.91 | ATLVT012XX1348 | 46282 | SGESAASSNDNA | −1.81 | −0.73 | −0.9 | −0.04 |
| ATLVT012XX1303 | 46237 | SGEAAASTNDNA | −1.95 | −1.21 | 0.47 | 1.72 | ATLVT012XX1349 | 46283 | SGESAASSNDNH | 0.36 | 2.16 | 0.47 | −0.07 |
| ATLVT012XX1304 | 46238 | SGEAAASTNDNH | −1.2 | 0.67 | −0.85 | 1.17 | ATLVT012XX1350 | 46284 | SGESAASSNDNT | −0.91 | 0.53 | 0.01 | −2.59 |
| ATLVT012XX1305 | 46239 | SGEAAASTNQNT | −0.13 | −0.87 | −1.47 | −1.79 | ATLVT012XX1351 | 46285 | SGESAASTNDNA | 0.7 | 0.93 | 0.25 | −1.36 |
| ATLVT012XX1306 | 46240 | SGEAAGASNDNA | −2.17 | 1.96 | −0.22 | −2.53 | ATLVT012XX1352 | 46286 | SGESAASTNDNH | −0.48 | 0.56 | 0.27 | −0.95 |
| ATLVT012XX1307 | 46241 | SGEAAGASNDNH | −1.17 | 2.53 | −0.97 | −2.08 | ATLVT012XX1353 | 46287 | SGESAASTNDNT | −0.28 | −1.09 | 0.58 | −1.74 |
| ATLVT012XX1308 | 46242 | SGEAAGASNDNT | −1.66 | 3.35 | −1.19 | −1.94 | ATLVT012XX1354 | 46288 | SGESAGASNDNA | 0.72 | −0.94 | −0.92 | −2.62 |
| ATLVT012XX1309 | 46243 | SGEAAGATNDNA | −2.19 | −0.04 | 0.55 | −0.18 | ATLVT012XX1355 | 46289 | SGESAGASNDNH | 1.43 | 0.44 | −0.53 | −1.88 |
| ATLVT012XX1310 | 46244 | SGEAAGATNDNH | 0.17 | 4.41 | −1.78 | 0.58 | ATLVT012XX1356 | 46290 | SGESAGASNDNT | 3.23 | −0.87 | 0.11 | −2.19 |
| ATLVT012XX1311 | 46245 | SGEAAGATNDNT | 0.14 | 1.67 | −0.27 | 0.67 | ATLVT012XX1357 | 46291 | SGESAGATNDNA | 0.93 | −1.1 | 0.07 | 0.21 |
| ATLVT012XX1312 | 46246 | SGEAAGSSNDNA | −0.35 | −1.21 | −1.49 | −0.56 | ATLVT012XX1358 | 46292 | SGESAGATNDNH | 2.37 | 1.82 | −0.5 | 0.17 |
| ATLVT012XX1313 | 46247 | SGEAAGSSNDNH | −0.79 | −1.1 | −0.13 | −0.13 | ATLVT012XX1359 | 46293 | SGESAGATNDNT | 2.02 | 2.53 | −0.73 | −1.71 |
| ATLVT012XX1314 | 46248 | SGEAAGSENDNT | 1.07 | 1.13 | −0.72 | −0.79 | ATLVT012XX1360 | 46294 | SGESAGSSNDNA | 1.84 | 0.35 | −0.38 | −0.85 |
| ATLVT012XX1315 | 46249 | SGEAAGSTNDNA | 1.32 | 1.4 | −0.34 | 0.68 | ATLVT012XX1361 | 46295 | SGESAGSSNDNH | 0.08 | −1.75 | 0.26 | −0.58 |
| ATLVT012XX1316 | 46250 | SGEAAGSTNDNH | 1.29 | 1.87 | −1.1 | 1.49 | ATLVT012XX1362 | 46296 | SGESAGSSNDNT | 0.56 | −1.41 | 0.56 | −1.14 |
| ATLVT012XX1317 | 46251 | SGEAAGSTNDNT | 1.15 | 0.71 | 1.39 | 0.77 | ATLVT012XX1363 | 46297 | SGESAGSTNDNA | 0.1 | 1.33 | 1.18 | −1.3 |
| ATLVT012XX1318 | 46252 | SGEAGAASNDNA | −0.42 | −0.41 | 0.51 | −1.67 | ATLVT012XX1364 | 46298 | SGESAGSTNDNH | 1.77 | 2 | 0.27 | 0.64 |
| ATLVT012XX1319 | 46253 | SGEAGAASNDNH | −0.93 | 2.62 | −1.22 | −0.97 | ATLVT012XX1365 | 46299 | SGESAGSTNDNT | 1.75 | −0.4 | 0.16 | 1.45 |
| ATLVT012XX1320 | 46254 | SGEAGAASNDNT | −2.17 | −1.21 | −0.74 | −2.7 | ATLVT012XX1366 | 46300 | SGESGAASNDNA | −1.18 | −0.57 | −0.6 | −1.9 |
| ATLVT012XX1321 | 46255 | SGEAGAATNDNA | −1.97 | 2.87 | −0.55 | 1.03 | ATLVT012XX1367 | 46301 | SGESGAASNDNH | 0.94 | 1 | −1.07 | −0.85 |
| ATLVT012XX1322 | 46256 | SGEAGAATNDNH | −1.18 | 1.77 | 0.8 | 1.6 | ATLVT012XX1368 | 46302 | SGESGAASNDNT | −0.96 | −0.85 | 0.43 | −1.01 |
| ATLVT012XX1323 | 46257 | SGEAGAATNDNT | −2.14 | −0.15 | −0.55 | 0.59 | ATLVT012XX1369 | 46303 | SGESGAATNDNA | −0.89 | 0.88 | −0.14 | −1.55 |
| ATLVT012XX1324 | 46258 | SGEAGASSNDNA | 1.63 | 1.66 | 0.73 | 0.06 | ATLVT012XX1370 | 46304 | SGESGAATNDNH | −1.06 | 0.53 | −0.52 | 0.93 |
| ATLVT012XX1325 | 46259 | SGEAGASSNDNH | −0.93 | −1.07 | −0.02 | −0.02 | ATLVT012XX1371 | 46305 | SGESGAATNDNT | −0.8 | 0.06 | 0.34 | 0.19 |
| ATLVT012XX1326 | 46260 | SGEAGASSNDNT | 0.7 | −1.67 | 0.56 | 0.74 | ATLVT012XX1372 | 46306 | SGESGASSNDNA | 0.6 | 0.66 | −0.53 | −0.84 |
| ATLVT012XX1327 | 46261 | SGEAGASTNDNA | −0.44 | 3.51 | −0.99 | −0.77 | ATLVT012XX1373 | 46307 | SGESGASSNDNH | −0.54 | −0.85 | −0.32 | −1.57 |
| ATLVT012XX1328 | 46262 | SGEAGASTNDNH | −0.79 | 1.12 | −0.77 | 1.69 | ATLVT012XX1374 | 46308 | SGESGASSNDNT | 0.16 | −0.46 | −0.28 | −0.91 |
| ATLVT012XX1329 | 46263 | SGEAGASTNDNT | −1 | 0.71 | 0.34 | 0.09 | ATLVT012XX1375 | 46309 | SGESGASTNDNA | −0.48 | 1.47 | −0.3 | −0.81 |
| ATLVT012XX1330 | 46264 | SGEAGGASNDNA | 3.14 | 2.38 | 0.35 | 1.4 | ATLVT012XX1376 | 46310 | SGESGASTNDNH | −1.9 | 0.44 | −0.63 | 1.12 |
| ATLVT012XX1331 | 46265 | SGEAGGASNDNH | 5.34 | 5.54 | 1.11 | 3.28 | ATLVT012XX1377 | 46311 | SGESGASTNDNT | −0.09 | −1.4 | −0.05 | 0.85 |
| ATLVT012XX1332 | 46266 | SGEAGGASNDNT | 2.74 | 5.44 | 0.56 | 1.55 | ATLVT012XX1378 | 46312 | SGESGGASNDNA | 3.04 | 1.99 | 1.24 | 1.66 |
| ATLVT012XX1333 | 46267 | SGEAGGATNDNA | 4.67 | 4.89 | 0.93 | 1.24 | ATLVT012XX1379 | 46313 | SGESGGASNHNH | 3.53 | 4.21 | 1.15 | 3.62 |
| ATLVT012XX1334 | 46268 | SGEAGGATNDNH | 9.25 | 11.42 | 1.08 | 4.43 | ATLVT012XX1380 | 46314 | SGESGGASNDNT | 2.7 | 2.45 | 1.59 | 0.48 |
| ATLVT012XX1381 | 46315 | SGESGGATNDNA | 3.86 | 4.42 | 1.63 | 1.05 | ATLVT012XX1427 | 46361 | SGETGGASNDNH | 3.1 | 4.03 | 0.34 | 2.04 |
| ATLVT012XX1382 | 46316 | SGESGGATNDNH | 8.54 | 9.82 | 1.52 | 4.17 | ATLVT012XX1428 | 46362 | SGETGGASNDNT | 1.7 | 2.85 | 0.52 | 0.67 |
| ATLVT012XX1383 | 46317 | SGESGGATNDNT | 4.43 | 4.03 | 0.74 | 0.88 | ATLVT012XX1429 | 46363 | SGETGGATNDNA | 2.98 | 2.67 | −0.06 | 2.82 |
| ATLVT012XX1384 | 46318 | SGESGGSSNDNA | 1.38 | 3.89 | 0.81 | 0.96 | ATLVT012XX1430 | 46364 | SGETGGATNDNH | 7.39 | 8.19 | 1.77 | 4.17 |
| ATLVT012XX1385 | 46319 | SGESGGSSNDNH | 1.38 | 2.62 | 0.85 | 2.32 | ATLVT012XX1431 | 46365 | SGETGGATNDNT | 3.41 | 2.73 | 0.75 | 2.75 |
| ATLVT012XX1386 | 46320 | SGESGGSSNDNT | 2.82 | 2.4 | 0.03 | 0.78 | ATLVT012XX1432 | 46366 | SGETGGSSNDNA | 0.29 | 2.33 | −0.3 | 1.59 |
| ATLVT012XX1387 | 46321 | SGESGGSTNDNA | 3.09 | 2.78 | 0.29 | 2.05 | ATLVT012XX1433 | 46367 | SGETGGSSNDNH | 2.01 | 1.15 | −0.1 | −0.53 |
| ATLVT012XX1388 | 46322 | SGESGGSTNDNH | 4.34 | 4 | 0.64 | 2.73 | ATLVT012XX1434 | 46368 | SGETGGSSNDNT | 2.15 | 0.63 | 0.54 | −0.49 |
| ATLVT012XX1389 | 46323 | SGESGGSTNDNT | 3.52 | 1.65 | 0.54 | 2.7 | ATLVT012XX1435 | 46369 | SGETGGSTNDNA | 1.65 | 2.25 | 0.78 | 2.27 |
| ATLVT012XX1390 | 46324 | SGETAAASNDNA | 2.45 | −2.56 | 0.66 | 0.11 | ATLVT012XX1436 | 46370 | SGETGGSTNDNH | 1.91 | 2.34 | 0.3 | 2.09 |
| ATLVT012XX1391 | 46325 | SGETAAASNDNH | 0 | −0.13 | −0.31 | −0.73 | ATLVT012XX1437 | 46371 | SGETGGSTNDNT | 2.07 | 1.05 | 0.98 | 1.29 |
| ATLVT012XX1392 | 46326 | SGETAAASNDNT | −0.15 | −1.8 | −0.35 | 0.37 | ATLVT012XX1438 | 46372 | SGQAAAASNDNA | 2.83 | 2.52 | 0.14 | −0.93 |
| ATLVT012XX1393 | 46327 | SGETAAATNDNA | 1.38 | −0.94 | −0.07 | 1.21 | ATLVT012XX1439 | 46373 | SGQAAAASNDNH | 1.57 | 2.87 | −0.53 | −0.24 |
| ATLVT012XX1394 | 46328 | SGETAAATNDNH | −0.74 | −1.31 | 0.14 | −0.22 | ATLVT012XX1440 | 46374 | SGQAAAASNDNT | 0.54 | 1.7 | 0.38 | 2.22 |
| ATLVT012XX1395 | 46329 | SGETAAATNDNT | 0.86 | −1.04 | 0.42 | 0.41 | ATLVT012XX1441 | 46375 | SGQAAAATNDNA | −0.49 | 1.42 | −0.81 | 0.24 |
| ATLVT012XX1396 | 46330 | SGETAASSNDNA | 0.13 | 1 | 1.39 | −0.75 | ATLVT012XX1442 | 46376 | SGQAAAATNDNH | 0.91 | 0.23 | 2.13 | 1.81 |
| ATLVT012XX1397 | 46331 | SGETAASSNDNH | −0.89 | 1.4 | −1.05 | 0.08 | ATTVT012XX1443 | 46377 | SGQAAAATNDNT | 0.64 | 1.15 | 1.52 | 0.33 |
| ATLVT012XX1398 | 46332 | SGETAASSNDNT | 1.7 | 1.61 | −0.44 | 0.43 | ATLVT012XX1444 | 46378 | SGQAAASSNDNA | 1.75 | 2.97 | 0.52 | −1.38 |
| ATLVT012XX1399 | 46333 | SGETAASTNDNA | 1 | 2.59 | 0.31 | 0.09 | ATLVT012XX1445 | 46379 | SGQAAASSNDNH | 1.85 | 3.36 | 0.95 | −0.14 |
| ATLVT012XX1400 | 46334 | SGETAASTNDNH | −0.9 | −0.4 | 0.18 | −0.04 | ATLVT012XX1446 | 46380 | SGQAAASSNDNT | 2.43 | 2.35 | −0.18 | 1.19 |
| ATLVT012XX1401 | 46335 | SGETAASTNDNT | 0.34 | −1.05 | 0.29 | −1.45 | ATLVT012XX1447 | 46381 | SGQAAASTNDNA | 2.41 | 4.06 | 0.86 | 0.69 |
| ATLVT012XX1402 | 46336 | SGETAGASNDNA | −1 | 0.68 | −1 | −0.23 | ATLVT012XX1448 | 46382 | SGQAAASTNDNH | 1.75 | 0.92 | 0.3 | 1.54 |
| ATLVT012XX1403 | 46337 | SGETAGASNDNH | −1.58 | 2.13 | −2.4 | −0.21 | ATLVT012XX1449 | 46383 | SGQAAASTNDNT | 1.74 | −0.02 | −0.37 | 2.36 |
| ATLVT012XX1404 | 46338 | SGETAGASNDNT | −1.59 | 2.35 | −0.96 | 0.02 | ATLVT012XX1450 | 46384 | SGQAAGASNDNA | −0.09 | 0.95 | −1.25 | −2.15 |
| ATLVT012XX1405 | 46339 | SGETAGATNDNA | −1.7 | 1.49 | −0.67 | 0.71 | ATLVT012XX1451 | 46385 | SGQAAGASNDNH | 1.61 | 2.3 | −1.64 | −1.54 |
| ATLVT012XX1406 | 46340 | SGETAGATNDNH | −1.21 | 1.62 | −0.02 | 2.81 | ATLVT012XX1452 | 46386 | SGQAAGASNDNT | 0.47 | 0.88 | −2.6 | −1.91 |
| ATLVT012XX1407 | 46341 | SGETAGATNDNT | −0.32 | −0.56 | −0.04 | 1.03 | ATLVT012XX1453 | 46387 | SGQAAGATNDNA | 2.62 | 0.72 | −1.04 | −0.18 |
| ATLVT012XX1408 | 46342 | SGETAGSSNDNA | −0.22 | 1.09 | −0.28 | −2.49 | ATLVT012XX1454 | 46388 | SGQAAGATNDNH | 5.95 | 3.61 | −0.8 | 3.4 |
| ATLVT012XX1409 | 46343 | SGETAGSSNDNH | −2.15 | 1.25 | 0.3 | −2.31 | ATLVT012XX1455 | 46389 | SGQAAGATNDNT | 3.96 | 2.18 | −1.46 | 0.93 |
| ATLVT012XX1410 | 46344 | SGETAGSSNDNT | −0.16 | −0.33 | −0.1 | −0.93 | ATLVT012XX1456 | 46390 | SGQAAGSSNDNA | −0.99 | 0.32 | 0.12 | 0.32 |
| ATLVT012XX1411 | 46345 | SGETAGSTNDNA | −0.4 | −0.86 | 0.31 | 0.2 | ATLVT012XX1457 | 46391 | SGQAAGSSNDNH | −0.91 | −0.58 | −0.51 | 0.34 |
| ATLVT012XX1412 | 46346 | SGETAGSTNDNH | 0.66 | 1.4 | −0.48 | −0.4 | ATLVT012XX1458 | 46392 | SGQAAGSSNDNT | −0.2 | 0.97 | −0.18 | 1 |
| ATLVT012XX1413 | 46347 | SGETAGSTNDNT | 0.21 | −0.62 | −0.87 | −0.68 | ATLVT012XX1459 | 46393 | SGQAAGSTNDNA | 0.67 | 2.2 | 0.56 | 0.65 |
| ATLVT012XX1414 | 46348 | SGETGAASNDNA | −1.1 | −0.85 | −1.37 | −1.97 | ATLVT012XX1460 | 46394 | SGQAAGSTNDNH | 2.21 | 6.12 | −0.5 | 1.94 |
| ATLVT012XX1415 | 46349 | SGETGAASNDNH | −1.35 | 3.54 | −1.34 | −0.57 | ATLVT012XX1461 | 46395 | SGQAAGSTNDNT | 2.5 | 5.3 | 0.55 | 2.55 |
| ATLVT012XX1416 | 46350 | SGETGAASNDNT | −1.26 | −0.07 | −2.43 | −2.05 | ATLVT012XX1462 | 46396 | SGQAGAASNDNA | 1.52 | 1.27 | −0.4 | −0.94 |
| ATLVT012XX1417 | 46351 | SGETGAATNDNA | −0.16 | 0.99 | −0.17 | 0.32 | ATLVT012XX1463 | 46397 | SGQAGAASNDNH | 0.63 | 1.19 | −0.93 | 0.65 |
| ATLVT012XX1418 | 46352 | SGETGAATNDNH | 1.49 | 0.14 | −1.37 | 2.14 | ATLVT012XX1464 | 46398 | SGQAGAASNDNT | 0.94 | 0.29 | −1.45 | −0.65 |
| ATLVT01XXX1419 | 46353 | SGETGAATNDNT | 1.1 | −0.6 | 0.1 | −1.72 | ATLVT012XX1465 | 46399 | SGQAGAATNDNA | 0.74 | 0.04 | −1.28 | 0.64 |
| ATLVT012XX1420 | 46354 | SGETGASSNDNA | −1.85 | 2.28 | 0.67 | −1.49 | ATLVT012XX1466 | 46400 | SGQAGAATNDNH | 2.42 | 5.53 | −0.29 | 1.82 |
| ATLVT012XX1421 | 46355 | SGETGASSNDNH | −0.49 | 0.25 | −0.65 | −1.26 | ATLVT012XX1467 | 46401 | SGQAGAATNDNT | 0.97 | 0.43 | 0.06 | 1.31 |
| ATLVT012XX1422 | 46356 | SGETGASSNDNT | −2.23 | 2.18 | −0.67 | −1.14 | ATLVT012XX1468 | 46402 | SGQAGASSNDNA | 2.58 | 1.2 | 0.48 | 0.73 |
| ATLVT012XX1423 | 46357 | SGETGASTNDNA | 0.04 | 1.55 | 0.75 | 0.43 | ATLVT012XX1469 | 46403 | SGQAGASSNDNH | 1.38 | 1.25 | −0.21 | 0.21 |
| ATLVT012XX1424 | 46358 | SGETGASTNDNH | −0.19 | 0.67 | 1.04 | 0.65 | ATLVT012XX1470 | 46404 | SGQAGASSNDNT | 3.48 | 1.3 | −0.64 | −0.78 |
| ATLVT012XX1425 | 46359 | SGETGASTNDNT | −0.26 | 0.04 | −0.55 | 0.61 | ATLVT012XX1471 | 46405 | SGQAGASTNDNA | 0.46 | 1.23 | 1.35 | 1.29 |
| ATLVT012XX1426 | 46360 | SGETGGASNDNA | 0.72 | 1.58 | 0.1 | 2.07 | ATLVT012XX1472 | 46406 | SGQAGASTNDNH | 1.79 | 1.38 | 0.78 | 1.86 |
| ATLVT012XX1473 | 46407 | SGQAGASTNDNT | 0.17 | 1.7 | 0.99 | 0.11 | ATLVT012XX1519 | 46453 | SGQSGASTNDNA | 0.58 | 1.94 | 0.29 | 0.6 |
| ATLVT01XXX1474 | 46408 | SGQAGGASNDNA | 4.39 | 6.08 | 0.04 | 2.13 | ATLVT012XX1520 | 46454 | SGQSGASTNDNH | 3.56 | 2.5 | 1.58 | 1.79 |
| ATLVT012XX1475 | 46409 | SGQAGGASNDNH | 9.33 | 11.99 | 1.57 | 5.23 | ATLVT012XX1521 | 46455 | SGQSGASTNDNT | 3.41 | 2.85 | 0.66 | 0.85 |
| ATLVT012XX1476 | 46410 | SGQAGGASNDNT | 6.17 | 6.69 | 0.82 | 3.04 | ATLVT012XX1522 | 46456 | SGQSGGASNDNA | 2.5 | 4.63 | 2.1 | 2.77 |
| ATLVT012XX1477 | 46411 | SGQAGGATNDNA | 4.7 | 5.79 | 1.04 | 3.28 | ATLVT012XX1523 | 46457 | SGQSGGASNDNH | 8.17 | 8.18 | 1.83 | 5 |
| ATLVT012XX1478 | 46412 | SGQAGGATNDNH | 9.92 | 11.89 | −0.11 | 3.72 | ATLVT012XX1524 | 46458 | SGQSGGASNDNT | 4.2 | 5.92 | 0.51 | 3.2 |
| ATLVT012XX1479 | 46413 | SGQAGGATNDNT | 4.96 | 5.93 | 1.41 | 4.5 | ATLVT012XX1525 | 46459 | SGQSGGATNDNA | 5.3 | 7.22 | 1.04 | 3.04 |
| ATLVT012XX1480 | 46414 | SGQAGGSSNDNA | 3.67 | 4.19 | 1.19 | 3.41 | ATLVT012XX1526 | 46460 | SGQSGGATNDNH | 9.86 | 11.99 | −0.06 | 4.21 |
| ATLVT012XX1481 | 46415 | SGQAGGSSNDNH | 5.17 | 5.87 | 0.99 | 3.66 | ATLVT012XX1527 | 46461 | SGQSGGATNDNT | 5.99 | 5.7 | 0.76 | 3.06 |
| ATLVT012XX1482 | 46416 | SGQAGGSSNDNT | 5.66 | 4.25 | 0.82 | 2.21 | ATLVT012XX1528 | 46462 | SGQSGGSSNDNA | 2.31 | 3.89 | 0.3 | 2.06 |
| ATLVT012XX1483 | 46417 | SGQAGGSTNDNA | 5.55 | 7.11 | 0.82 | 4.29 | ATLVT012XX1529 | 46463 | SGQSGGSSNDNH | 3.49 | 3.83 | 0.73 | 3.29 |
| ATLVT012XX1484 | 46418 | SGQAGGSTNDNH | 8.44 | 9.89 | 1.46 | 4.4 | ATLVT012XX1530 | 46464 | SGQSGGSSNDNT | 3.85 | 3.38 | 0.85 | 1.45 |
| ATLVT012XX1485 | 46419 | SGQAGGSTNDNT | 7.08 | 7.73 | 1.73 | 4.18 | ATLVT012XX1531 | 46465 | SGQSGGSTNDNA | 4.66 | 6.66 | 0.63 | 4.19 |
| ATLVT012XX1486 | 46420 | SGQSAAASNDNA | 3.54 | 3.45 | 0.07 | 0.08 | ATLVT012XX1532 | 46466 | SGQSGGSTNDNH | 8.12 | 10.05 | 1.36 | 4.5 |
| ATLVT012XX1487 | 46421 | SGQSAAASNDNH | 3.53 | 2.22 | −0.14 | 1.14 | ATLVT012XX1533 | 46467 | SGQSGGSTNDNT | 5.92 | 6.31 | 0.58 | 4.12 |
| ATLVT012XX1488 | 46422 | SGQSAAASNDNT | 0.72 | 2.05 | 0.56 | 1.59 | ATLVT012XX1534 | 46468 | SGQTAAASNDNA | 1.11 | 0.37 | −0.55 | 1.06 |
| ATLVT012XX1489 | 46423 | SGQSAAATNDNA | −0.34 | 3.61 | −0.08 | −0.59 | ATTVT012XX1535 | 46469 | SGQTAAASNDNH | 1.26 | 2.93 | −0.37 | 1.12 |
| ATLVT012XX1490 | 46424 | SGQSAAATNDNH | 0.97 | 2.55 | 0.24 | 1.68 | ATLVT012XX1536 | 46470 | SGQTAAASNDNT | 0.8 | 0.17 | −0.63 | −0.42 |
| ATLVT012XX1491 | 46425 | SGQSAAATNDNT | 0.29 | 3.39 | 0.75 | 1.2 | ATLVT012XX1537 | 46471 | SGQTAAATNDNA | 0.95 | −0.44 | 1.07 | 1.99 |
| ATLVT012XX1492 | 46426 | SGQSAASSNDNA | 0.44 | 0.57 | −0.81 | 1.52 | ATLVT012XX1538 | 46472 | SGQTAAATNDNH | 1.57 | 1.03 | 1.06 | 0.53 |
| ATLVT012XX1493 | 46427 | SGQSAASSNDNH | 1.17 | −0.84 | 0.67 | −0.76 | ATLVT012XX1539 | 46473 | SGQTAAATNDNT | 2.27 | −0.47 | 0.83 | 0.95 |
| ATLVT012XX1494 | 46428 | SGQSAASSNDNT | 1.37 | −1.32 | 0.56 | 0.67 | ATLVT012XX1$40 | 46474 | SGQTAASSNDNA | 0.45 | 1.7 | 0.53 | 0.77 |
| ATLVT012XX1495 | 46429 | SGQSAASTNDNA | 0.38 | 1.23 | 1.42 | 0.1 | ATLVT012XX1541 | 46475 | SGQTAASSNDNH | −0.19 | 1.82 | 0.89 | 0.46 |
| ATLVT012XX1496 | 46430 | SGQSAASTNDNH | −0.35 | 1.15 | −0.84 | 1.17 | ATLVT012XX1542 | 46476 | SGQTAASSNDNT | 0.82 | 2.53 | 0.32 | 0.07 |
| ATLVT012XX1497 | 46431 | SGQSAASTNDNT | 1.35 | 1.19 | 0.92 | 1.52 | ATLVT012XX1543 | 46477 | SGQTAASTNDNA | −0.82 | −0.68 | 0.48 | 1.83 |
| ATLVT012XX1498 | 46432 | SGQSAGASNDNA | 2.14 | 1.47 | −0.52 | −0.35 | ATLVT012XX1544 | 46478 | SGQTAASTNDNH | −0.02 | 0.98 | −0.15 | 2.75 |
| ATLVT012XX1499 | 46433 | SGQSAGASNDNH | 2.46 | 2.16 | 0.62 | 1.82 | ATLVT012XX1545 | 46479 | SGQTAASTNDNT | 0.7 | 2.26 | 1.35 | 1.3 |
| ATLVT012XX1500 | 46434 | SGQSAGASNDNT | 1.57 | 2.41 | −1.14 | 0.68 | ATLVT012XX1546 | 46480 | SGQTAGASNDNA | −0.37 | 1.8 | 1.11 | −1.08 |
| ATLVT012XX1501 | 46435 | SGQSAGATNDNA | 1.89 | 4.37 | 0.26 | 2.86 | ATLVT012XX1547 | 46481 | SGQTAGASNDNH | 0.58 | 0.43 | 0.69 | 2.66 |
| ATLVT012XX1502 | 46436 | SGQSAGATNDNH | 6.23 | 6.22 | 0.73 | 5.07 | ATLVT012XX1548 | 46482 | SGQTAGASNDNT | 0.35 | 0.24 | 0.87 | 0.9 |
| ATLVT012XX1503 | 46437 | SGQSAGATNDNT | 3.61 | 3.34 | −0.52 | 2.19 | ATLVT012XX1549 | 46483 | SGQTAGATNDNA | −0.65 | 0.55 | −0.45 | 0.11 |
| ATLVT012XX1504 | 46438 | SGQSAGSSNDNA | 1.33 | 4.55 | −1.14 | −1.08 | ATLVT012XX1550 | 46484 | SGQTAGATNDNH | 4.66 | 7.04 | 0.86 | 4.11 |
| ATLVT012XX1505 | 46439 | SGQSAGSSNDNH | 1.9 | 4 | 0.44 | 0.82 | ATLVT012XX1551 | 46485 | SGQTAGATNDNT | 0.04 | −0.02 | 0.6 | 0.81 |
| ATLVT012XX1506 | 46440 | SGQSAGSSNDNT | 1.4 | 2.58 | 0.88 | 1.45 | ATLVT012XX1552 | 46486 | SGQTAGSSNDNA | −1.03 | 1.91 | 0.28 | 0.43 |
| ATLVT012XX1507 | 46441 | SGQSAGSTNDNA | 1.87 | 2.74 | 0.25 | 3.1 | ATLVT012XX1553 | 46487 | SGQTAGSSNDNH | 0.02 | 0.04 | 1.24 | 1.63 |
| ATLVT012XX1508 | 46442 | SGQSAGSTNDNH | 1.55 | 4.49 | −0.23 | 3.61 | ATLVT012XX1554 | 46488 | SGQTAGSSNDNT | −0.87 | 1.36 | 0.7 | −0.25 |
| ATLVT012XX1509 | 46443 | SGQSAGSTNDNT | 2.65 | 3.29 | −0.49 | 2.07 | ATLVT012XX1555 | 46489 | SGQTAGSTNDNA | 0.39 | 2.32 | −1.07 | 1.49 |
| ATLVT012XX1510 | 46444 | SGQSGAASNDNA | 2.63 | −0.37 | −2.18 | −1.35 | ATLVT012XX1556 | 46490 | SGQTAGSTNDNH | 3.01 | 1.88 | 0.85 | 3.65 |
| ATLVT012XX1511 | 46445 | SGQSGAASNDNH | 2.16 | 0.7 | −0.2 | −1.79 | ATLVT012XX1557 | 46491 | SGQTAGSTNDNT | 0.62 | 3.86 | −0.08 | 1.82 |
| ATLVT012XX1512 | 46446 | SGQSGAASNDNT | 3 | −0.69 | −1.92 | 0.41 | ATLVT012XX1558 | 46492 | SGQTGAASNDNA | 0.82 | −1.51 | 0.26 | 2.32 |
| ATLVT012XX1513 | 46447 | SGQSGAATNDNA | 2.65 | 1 | 0.16 | 1.11 | ATLVT012XX1559 | 46493 | SGQTGAASNDNH | 2.14 | −0.83 | 0.95 | 0.61 |
| ATLVT012XX1514 | 46448 | SGQSGAATNDNH | 2.69 | 3.4 | 1.14 | 3.52 | ATLVT012XX1560 | 46494 | SGQTGAASNDNT | 2.73 | 1.11 | 0.44 | 0.5 |
| ATLVT012XX1515 | 46449 | SGQSGAATNDNT | 2.99 | 1.63 | −0.14 | 1.95 | ATLVT012XX1561 | 46495 | SGQTGAATNDNA | 0.1 | 0.16 | −0.38 | −0.2 |
| ATLVT012XX1516 | 46450 | SGQSGASSNDNA | 2.13 | 2.72 | 1.08 | −0.56 | ATLVT012XX1562 | 46496 | SGQTGAATNDNH | 2.8 | 2.74 | −0.79 | 3.63 |
| ATLVT012XX1517 | 46451 | SGQSGASSNDNH | 2.37 | 2.92 | 1.4 | −0.86 | ATLVT012XX1563 | 46497 | SGQTGAATNDNT | 1.21 | 0.41 | −0.44 | 0.08 |
| ATLVT012XX1518 | 46452 | SGQSGASSNDNT | 1.84 | −0.23 | 0.98 | −0.08 | ATLVT012XX1564 | 46498 | SGQTGASSNDNA | 1.06 | 1.5 | 0.29 | 0.11 |
| ATLVT012XX1565 | 46499 | SGQTGASSNDNH | 2.55 | 1.64 | 1.09 | −0.27 | ATTVT012XX1611 | 46545 | SGTAGAATNDNT | −0.8. | 2.13 | 0.04 | 1.15 |
| ATLVT012XX1566 | 46500 | SGQTGASSNDNT | 1.76 | −0.35 | 0.2 | −1.2 | ATLVT012XX1612 | 46546 | SGTAGASSNDNA | −0.08 | 0.47 | −0.36 | 2.21 |
| ATLVT012XX1567 | 46501 | SGQTGASTNDNA | 0.68 | 3.56 | 0.11 | 2.24 | ATLVT012XX1613 | 46547 | SGTAGASSNDNH | 1.28 | 1.92 | 0.6 | 1.53 |
| ATLVT012XX1568 | 46502 | SGQTGASTNDNH | 1.56 | 4.91 | 0.59 | 2.05 | ATLVT012XX1614 | 46548 | SGTAGASSNDNT | 1.38 | −0.2 | 0.58 | −0.29 |
| ATLVT012XX1569 | 46503 | SGQTGASTNDNT | −0.06 | 0.72 | 0.46 | 1.33 | ATLVT012XX1615 | 46549 | SGTAGASTNDNA | 3.15 | 5.01 | 0.77 | 0.73 |
| ATLVT012XX1570 | 46504 | SGQTGGASNDNA | 3.13 | 4.08 | 0.9 | 3.3 | ATLVT012XX1616 | 46550 | SGTAGASTNDNH | 1.23 | 1.48 | −0.35 | 1.63 |
| ATLVT012XX1571 | 46505 | SGQTGGASNDNH | 6.54 | 8.15 | 1.31 | 3.5 | ATLVT012XX1617 | 46551 | SGTAGASTNDNT | 1.49 | 0.59 | 0.69 | 2.08 |
| ATLVT012XX1572 | 46506 | SGQTGGASNDNT | 4.02 | 5.98 | 0.78 | 0.72 | ATLVT012XX1618 | 46552 | SGTAGGASNDNA | 6 | 6.65 | 1.04 | 4.84 |
| ATLVT012XX1573 | 46507 | SGQTGGATNDNA | 3.68 | 4.25 | −0.26 | 3.4 | ATLVT012XX1619 | 46553 | SGTAGGASNDNH | 10.04 | 11.58 | 1.06 | 5.44 |
| ATLVT012XX1574 | 46508 | SGQTGGATNDNH | 9.6 | 12.38 | 0.01 | 4.01 | ATLVT012XX1620 | 46554 | SGTAGGASNDNT | 6.3 | 9.6 | 0.57 | 4.51 |
| ATLVT012XX1575 | 46509 | SGQTGGATNDNT | 3.97 | 6.43 | 1.44 | 2.93 | ATLVT012XX1621 | 46555 | SGTAGGATNDNA | 5.81 | 4.87 | −0.35 | 4.71 |
| ATLVT012XX1576 | 46510 | SGQTGGSSNDNA | 3.25 | 4.21 | 0.63 | 1.88 | ATLVT012XX1622 | 46556 | SGTAGGATNDNH | 9.89 | 10.6 | −0.51 | 3.05 |
| ATLVT012XX1577 | 46511 | SGQTGGSSNDNH | 3.37 | 4.17 | 1.49 | 2.66 | ATLVT012XX1623 | 46557 | SGTAGGATNDNT | 6.86 | 8.41 | 0.4 | 4.13 |
| ATLVT012XX1578 | 46512 | SGQTGGSSNDNT | 3.13 | 2.8 | −0.02 | 2.71 | ATLVT012XX1624 | 46558 | SGTAGGSSNDNA | 6.55 | 5.91 | 1.67 | 4.4 |
| ATLVT012XX1579 | 46513 | SGQTGGSTNDNA | 4.63 | 4.16 | 0.55 | 3.02 | ATLVT012XX1625 | 46559 | SGTAGGSSNDNH | 5.77 | 5.66 | −0.27 | 3.96 |
| ATLVT012XX1580 | 46514 | SGQTGGSTNDNH | 8 | 8.23 | 0.82 | 4.32 | ATLVT012XX1626 | 46560 | SGTAGGSSNDNT | 5.86 | 5.69 | 2.35 | 4.55 |
| ATLVT012XX1581 | 46515 | SGQTGGSTNDNT | 4.91 | 6.26 | 1.36 | 3.13 | ATLVT012XX1627 | 46561 | SGTAGGSTNDNA | 7.39 | 8.73 | 1.73 | 4.66 |
| ATLVT012XX1582 | 46516 | SGTAAAASNDNA | 0.52 | 1.83 | −0.47 | 0.96 | ATLVT012XX1628 | 46562 | SGTAGGSTNDNH | 8.81 | 8.85 | 1.86 | 4.08 |
| ATLVT012XX1583 | 46517 | SGTAAAASNDNH | 2.12 | 2.48 | 0.57 | 2.59 | ATLVT012XX1629 | 46563 | SGTAGGSTNDNT | 7.52 | 9 | 1.22 | 4.71 |
| ATLVT012XX1584 | 46518 | SGTAAAASNDNT | 2.79 | 3.5 | −0.51 | 1.69 | ATLVT012XX1630 | 46564 | SGTSAAASNDNA | 0.99 | −0.93 | −1.94 | −1.23 |
| ATLVT012XX1585 | 46519 | SGTAAAATNDNA | 2.02 | 2.12 | −0.09 | 0.52 | ATLVT012XX1631 | 46565 | SGTSAAASNDNH | 2.51 | 0.17 | −0.46 | 0.4 |
| ATLVT012XX1586 | 46520 | SGTAAAATNDNH | 2.03 | 3.38 | −1.21 | 1.01 | ATLVT012XX1632 | 46566 | SGTSAAASNDNT | 2.1 | −1.27 | −1 | −0.87 |
| ATLVT012XX1587 | 46521 | SGTAAAATNDNT | 0.6 | 2.1 | 0.2 | 0.65 | ATLVT012XX1633 | 46567 | SGTSAAATNDNA | 0.99 | −1.46 | −0.19 | 0 |
| ATLVT012XX1588 | 46522 | SGTAAASSNDNA | 3.98 | −0.89 | −0.75 | −0.18 | ATLVT012XX1634 | 46568 | SGTSAAATNDNH | 0.73 | −0.57 | −0.67 | 0.6 |
| ATLVT012XX1589 | 46523 | SGTAAASSNDNH | 0.57 | −0.34 | 0.36 | 0.18 | ATLVT012XX1635 | 46569 | SGTSAAATNDNT | 2.8 | −1.86 | 0.26 | 0.24 |
| ATLVT012XX1590 | 46524 | SGTAAASSNDNT | 3.5 | −0.14 | −0.31 | 0.22 | ATLVT012XX1636 | 46570 | SGTSAASSNDNA | 2.36 | −2.71 | 0.49 | 0 |
| ATLVT012XX1591 | 46525 | SGTAAASTNDNA | 2.23 | 1.29 | 0.71 | 1.13 | ATLVT012XX1637 | 46571 | SGTSAASSNDNH | 0.4 | −2.53 | −0.29 | −1.32 |
| ATLVT012XX1592 | 46526 | SGTAAASTNDNH | 1.72 | 1.72 | 0.84 | 1.8 | ATLVT012XX1638 | 46572 | SGTSAASSNDNT | 1.82 | −0.67 | −0.06 | −1.15 |
| ATLVT012XX1593 | 46527 | SGTAAASTNDNT | 0.31 | 2.65 | 0.26 | 0.89 | ATLVT012XX1639 | 46573 | SGTSAASTNDNA | 0.15 | 1.92 | 0.18 | −0.61 |
| ATLVT012XX1594 | 46528 | SGTAAGASNDNA | 1.73 | 2.27 | 0.03 | 0.78 | ATLVT012XX1640 | 46574 | SGTSAASTNDNH | −0.14 | 0.42 | 0.26 | 0.36 |
| ATLVT012XX1595 | 46529 | SGTAAGASNDNH | 2.28 | 1.51 | 0.01 | 1.68 | ATLVT012XX1641 | 46575 | SGTSAASTNDNT | 0.68 | −0.76 | 0.25 | 0.35 |
| ATLVT012XX1596 | 46530 | SGTAAGASNDNT | 3 | 3.19 | −0.97 | 3.35 | ATLVT012XX1642 | 46576 | SGTSAGASNDNA | 0.34 | 2.56 | −0.15 | 1.6 |
| ATLVT012XX1597 | 46531 | SGTAAGATNDNA | 3.15 | 1.97 | 0.42 | 0.07 | ATLVT012XX1643 | 46577 | SGTSAGASNDNH | 4.96 | 1.18 | 1.55 | 1.92 |
| ATLVT012XX1598 | 46532 | SGTAAGATNDNH | 6.83 | 7.77 | 0.6 | 4.28 | ATLVT012XX1644 | 46578 | SGTSAGASNDNT | 1.41 | 0.29 | 1.14 | 1.87 |
| ATLVT012XX1599 | 46533 | SGTAAGATNDNT | 2.15 | 2.95 | −0.31 | 1.19 | ATLVT012XX1645 | 46579 | SGTSAGATNDNA | 2.02 | 2.21 | −0.34 | 1.21 |
| ATLVT012XX1600 | 46534 | SGTAAGSSNDNA | 0.66 | 0.15 | 0.05 | 1.75 | ATLVT012XX1646 | 46580 | SGTSAGATNDNH | 6.42 | 9.05 | 0.96 | 4.38 |
| ATLVT012XX1601 | 46535 | SGTAAGSSNDNH | 0.24 | 1.46 | −0.57 | 0.69 | ATLVT012XX1647 | 46581 | SGTSAGATNDNT | 2.9 | 2.7 | 0.95 | 2.97 |
| ATLVT012XX1602 | 46536 | SGTAAGSSNDNT | −0.09 | 2.04 | −0.61 | 1.07 | ATTVT012XX1648 | 46582 | SGTSAGSSNDNA | 2.17 | 0.76 | −1.38 | 0.02 |
| ATLVT01XXX1603 | 46537 | SGTAAGSTNDNA | 0.97 | −0.18 | 0.9 | 1.71 | ATLVT012XX1649 | 46583 | SGTSAGSSNDNH | −0.08 | 0.05 | 0.14 | 1.43 |
| ATLVT012XX1604 | 46538 | SGTAAGSTNDNH | 0.54 | 2.43 | −0.75 | 3.61 | ATLVT012XX1650 | 46584 | SGTSAGSSNDNT | 1.86 | 0.61 | −0.35 | 0.96 |
| ATLVT012XX1605 | 46539 | SGTAAGSTNDNT | 1.04 | −0.29 | −0.08 | 1.69 | ATLVT012XX1651 | 46585 | SGTSAGSTNDNA | 2.56 | −0.2 | 0.79 | 1.8 |
| ATLVT012XX1606 | 46540 | SGTAGAASNDNA | 1.63 | 0.45 | −0.6 | 0.53 | ATLVT012XX1652 | 46586 | SGTSAGSTNDNH | 2.67 | 1.62 | 0.64 | 2.2 |
| ATLVT012XX1607 | 46541 | SGTAGAASNDNH | 0.57 | −0.03 | 0.21 | −1.44 | ATLVT012XX1653 | 46587 | SGTSAGSTNDNT | 2.68 | 1.36 | 1.03 | 2.02 |
| ATLVT012XX1608 | 46542 | SGTAGAASNDNT | 0.4 | −0.28 | −1.07 | −1.77 | ATLVT012XX1654 | 46588 | SGTSGAASNDNA | 0.84 | 0.68 | 1.29 | −1.83 |
| ATLVT012XX1609 | 46543 | SGTAGAATNDNA | 0.23 | 2.61 | −0.51 | −0.21 | ATLVT012XX1655 | 46589 | SGTSGAASNDNH | 1.05 | 0.38 | 0.43 | −0.85 |
| ATLVT012XX1610 | 46544 | SGTAGAATNDNH | 2.42 | 4.95 | −1.55 | 1.45 | ATLVT012XX1656 | 46590 | SGTSGAASNDNT | 1.09 | −0.65 | 0.84 | −2.72 |
| ATLVT012XX1657 | 46591 | SGTSGAATNDNA | 2.13 | 0.61 | −0.51 | 0.86 | ATLVT012XX1703 | 46637 | SGTTGAASNDNH | 0.47 | 2.09 | −1.46 | 0.66 |
| ATLVT012XX1658 | 46592 | SGTSGAATNDNH | 1.5 | 3.09 | −0.52 | 1.36 | ATLVT012XX1704 | 46638 | SGTTGAASNDNT | −0.39 | 1.52 | −0.04 | −1.27 |
| ATLVT012XX1659 | 46593 | SGTSGAATNDNT | −0.14 | −0.08 | 0.16 | −1.04 | ATLVT012XX1705 | 46639 | SGTTGAATNDNA | 3.03 | 1.52 | −0.51 | −1.63 |
| ATLVT012XX1660 | 46594 | SGTSGASSNDNA | 2.06 | 1.74 | 0.76 | −0.24 | ATLVT012XX1706 | 46640 | SGTTGAATNDNH | 4.17 | 4.56 | −0.86 | 1.54 |
| ATLVT012XX1661 | 46595 | SGTSGASSNDNH | 0.05 | 0.74 | 0.39 | −0.13 | ATLVT012XX1707 | 46641 | SGTTGAATNDNT | 1.48 | 0.77 | 1.1 | −2.12 |
| ATLVT012XX1662 | 46596 | SGTSGASSNDNT | 0.72 | 2.95 | −0.67 | 0.15 | ATLVT012XX1708 | 46642 | SGTTGASSNDNA | 1.17 | 1.02 | 0.38 | 0.45 |
| ATLVT012XX1663 | 46597 | SGTSGASTNDNA | −0.08 | 0.33 | 1.35 | 1.65 | ATLVT012XX1709 | 46643 | SGTTGASSNDNH | 2.36 | 1.13 | 1.09 | 1.08 |
| ATLVT012XX1664 | 46598 | SGTSGASTNDNH | 0.9 | 1.11 | 0.29 | 1.77 | ATLVT012XX1710 | 46644 | SGTTGASSNDNT | 1. | −1.17 | −0.32 | −0.09 |
| ATLVT012XX1665 | 46599 | SGTSGASTNDNT | 2.18 | 0.44 | 1.19 | −0.08 | ATLVT012XX1711 | 46645 | SGTTGASTNDNA | 1.26 | 0.84 | 0.74 | 2.46 |
| ATLVT012XX1666 | 46600 | SGTSGGASNDNA | 4.83 | 5.82 | 2.01 | 3.56 | ATLVT012XX1712 | 46646 | SGTTGASTNDNH | 2 | 1.92 | 0.46 | 0.73 |
| ATLVT012XX1667 | 46601 | SGTSGGASNDNH | 8.43 | 9.08 | 1.95 | 4.59 | ATLVT012XX1713 | 46647 | SGTTGASTNDNT | 1.05 | 2.31 | −0.49 | 2.03 |
| ATLVT012XX1668 | 46602 | SGTSGGASNDNT | 5.68 | 6.56 | 1.7 | 3.41 | ATLVT012XX1714 | 46648 | SGTTGGASNDNA | 3.43 | 6.07 | 0.69 | 4.26 |
| ATLVT012XX1669 | 46603 | SGTSGGATNDNA | 4.75 | 6.4 | 1.26 | 3.69 | ATLVT012XX1715 | 46649 | SGTTGGASNDNH | 7.38 | 9.99 | 1.57 | 4.6 |
| ATLVT012XX1670 | 46604 | SGTSGGATNDNH | 10.13 | 12.32 | 1.08 | 4.16 | ATLVT012XX1716 | 46650 | SGTTGGASNDNT | 4.4 | 5.75 | 1.26 | 2.61 |
| ATLVT012XX1671 | 46605 | SGTSGGATNDNT | 6.71 | 6.92 | 1.3 | 3.36 | ATLVT012XX1717 | 46651 | SGTTGGATNDNA | 4.71 | 7.88 | 0.5 | 2.97 |
| ATLVT012XX1672 | 46606 | SGTSGGSSNDNA | 4.17 | 5.83 | 0.54 | 3.47 | ATLVT012XX1718 | 46652 | SGTTGGATNDNH | 9.98 | 12.6 | 0.54 | 3.93 |
| ATLVT012XX1673 | 46607 | SGTSGGSSNDNH | 4.44 | 4.04 | 0.29 | 3.15 | ATLVT012XX1719 | 46653 | SGTTGGATNDNT | 5.54 | 6.78 | 0.98 | 2.47 |
| ATLVT012XX1674 | 46608 | SGTSGGSSNDNT | 4.36 | 4.31 | 0.96 | 2.04 | ATLVT012XX1720 | 46654 | SGTTGGSSNDNA | 2.91 | 3.33 | 0.26 | 3.27 |
| ATLVT012XX1675 | 46609 | SGTSGGSTNDNA | 6.06 | 5.89 | 0.78 | 3.95 | ATLVT012XX1721 | 46655 | SGTTGGSSNDNH | 1.87 | 4.21 | 0.51 | 2.9 |
| ATLVT012XX1676 | 46610 | SGTSGGSTNDNH | 7.19 | 9.17 | 0.76 | 4.1 | ATLVT012XX1722 | 46656 | SGTTGGSSNDNT | 4.01 | 5.43 | 0.54 | 2.99 |
| ATLVT012XX1677 | 46611 | SGTSGGSTNDNT | 6.04 | 6.78 | 0.53 | 3.6 | ATLVT012XX1723 | 46657 | SGTTGGSTNDNA | 5.37 | 6.35 | 1.64 | 3.74 |
| ATLVT012XX1678 | 46612 | SGTTAAASNDNA | 2.96 | 2.96 | 1.08 | 0.24 | ATLVT012XX1724 | 46658 | SGTTGGSTNDNH | 7 | 7.42 | 1.17 | 3.73 |
| ATLVT012XX1679 | 46613 | SGTTAAASNDNH | 1.33 | 0.59 | 0.38 | 0.38 | ATLVT012XX1725 | 46659 | SGTTGGSTNDNT | 5.83 | 5.77 | 1.3 | 3.32 |
| ATLVT012XX1680 | 46614 | SGTTAAASNDNT | 2.68 | 2.26 | 1.1 | 1.47 | ATLVT012XX1726 | 46660 | SSAAAAASNDNA | 1.78 | −2.15 | −0.29 | 1.59 |
| ATLVT012XX1681 | 46615 | SGTTAAANDNA | −0.26 | 1.68 | 0.73 | 0.78 | ATLVT012XX1727 | 46661 | SSAAAAASNDNH | 1.59 | −2.3 | −0.08 | 1.41 |
| ATLVT012XX1682 | 46616 | SGTTAAATNDNH | −0.01 | 1.03 | 1.18 | 1.26 | ATLVT012XX1728 | 46662 | SSAAAAASNDNT | 0.85 | −0.78 | 0.29 | −0.08 |
| ATLVT012XX1683 | 46617 | SGTTAAATNDNT | −0.66 | 0.67 | 0.65 | 1.67 | ATLVT012XX1729 | 46663 | SSAAAAATNDNA | 1.21 | −1.57 | 0.9 | 2.27 |
| ATLVT012XX1684 | 46618 | SGTTAASSNDNA | 0.45 | −1.66 | −0.64 | 0.89 | ATLVT012XX1730 | 46664 | SSAAAAATNDNH | 0.36 | −0.55 | 0.37 | 2.01 |
| ATLVT012XX1685 | 46619 | SGTTAASSNDNH | 0.62 | −2.59 | 0.73 | −1.07 | ATLVT012XX1731 | 46665 | SSAAAAATNDNT | 2.69 | 0.87 | −0.41 | 1.17 |
| ATLVT012XX1686 | 46620 | SGTTAASSNDNT | 0.58 | −0.83 | −0.85 | 0.88 | ATLVT012XX1732 | 46666 | SSAAAASSNDNA | 1.14 | −0.89 | −0.38 | 0.46 |
| ATLVT01XXX1687 | 46621 | SGTTAASTNDNA | 1.61 | 0.94 | 1.26 | −0.4 | ATLVT012XX1733 | 46667 | SSAAAASSNDNH | 1.84 | −1.9 | 0.15 | 0.42 |
| ATLVT012XX1688 | 46622 | SGTTAASTNDNH | 4 | 2.96 | 0.33 | 0.74 | ATLVT012XX1734 | 46668 | SSAAAASSNDNT | 1.87 | −1.59 | 0.79 | 0.66 |
| ATLVT012XX1689 | 46623 | SGTTAASTNDNT | 1.37 | 0.19 | 0.06 | 1 | ATLVT012XX1735 | 46669 | SSAAAASTNDNA | 1.51 | 0.29 | 0.48 | 1.1 |
| ATLVT012XX1690 | 46624 | SGTTAGASNDNA | 0.33 | 1.3 | −1.95 | 2.77 | ATLVT012XX1736 | 46670 | SSAAAASTNQNH | 1.06 | 2.37 | 0.01 | 1.3 |
| ATLVT012XX1691 | 46625 | SGTTAGASNDNH | 0.44 | 1.24 | 0.87 | 2.74 | ATLVT012XX1737 | 46671 | SSAAAASTNDNT | 1.87 | 1.81 | 0.67 | 0.15 |
| ATLVT012XX1692 | 46626 | SGTTAGASNDNT | 2.45 | 2.62 | −1.5 | 0.77 | ATLVT012XX1738 | 46672 | SSAAAGASNDNA | −0.28 | −0.74 | −0.81 | −1 |
| ATLVT012XX1693 | 46627 | SGTTAGATNDNA | 2.31 | 4.18 | −0.06 | 1.98 | ATLVT012XX1739 | 46673 | SSAAAGASNDNH | −1.76 | −2.58 | −1.22 | −0.45 |
| ATLVT012XX1694 | 46628 | SGTTAGATNDNH | 4.29 | 4.79 | 1.84 | 3.52 | ATLVT012XX1740 | 46674 | SSAAAGASNDNT | −1.72 | −2.03 | −0.62 | −1.75 |
| ATLVT01XXX1695 | 46629 | SGTTAGATNDNT | 1.9 | 2.25 | 0.37 | 3.15 | ATLVT012XX1741 | 46675 | SSAAAGATNDNA | 2.13 | −0.05 | −1.97 | −0.37 |
| ATLVT012XX1696 | 46630 | SGTTAGSSNDNA | 0.69 | −0.29 | 0.08 | 1.34 | ATLVT012XX1742 | 46676 | SSAAAGATNDNH | 1.03 | 1.64 | 0.14 | 1.73 |
| ATLVT012XX1697 | 46631 | SGTTAGSSNDNH | −0.04 | −0.75 | 0.13 | 0.34 | ATLVT012XX1743 | 46677 | SSAAAGATNDNT | −0.05 | −0.97 | 0.35 | 1 |
| ATLVT012XX1698 | 46632 | SGTTAGSSNDNT | 1.54 | 1.65 | −0.24 | 0.19 | ATLVT012XX1744 | 46678 | SSAAAGSSNDNA | −2.31 | −2.65 | 0.9 | −1.44 |
| ATLVT012XX1699 | 46633 | SGTTAGSTNDNA | 2.25 | −0.07 | 0.19 | −0.09 | ATLVT012XX1745 | 46679 | SSAAAGSSNDNH | −2.26 | −2.93 | −0.55 | 0.03 |
| ATLVT012XX1700 | 46634 | SGTTAGSTNDNH | 3.98 | 2.79 | 1.07 | 2.65 | ATLVT012XX1746 | 46680 | SSAAAGSSNDNT | −2.82 | −1.56 | −0.3 | −1.75 |
| ATLVT012XX1701 | 46635 | SGTTAGSTNDNT | 3.17 | 2.66 | 0.2 | 3.05 | ATLVT012XX1747 | 46681 | SSAAAGSTNDNA | 1.26 | 1.36 | −1.26 | −1.45 |
| ATLVT012XX1702 | 46636 | SGTTGAASNDNA | 0.69 | 0.4 | −0.97 | −2.21 | ATLVT012XX1748 | 46682 | SSAAAGSTNDNH | −1.02 | −1.38 | −0.79 | −0.36 |
| ATLVT012XX1749 | 46683 | SSAAAGSTNDNT | 1.45 | −0.56 | −2.43 | −2.34 | ATLVT012XX1795 | 46729 | SSASAGSTNDNA | 0.71 | −0.11 | −0.05 | −0.67 |
| ATLVT012XX1750 | 46684 | SSAAGAASNDNA | −2.27 | −1.25 | −1.49 | −3.73 | ATLVT012XX1796 | 46730 | SSASAGSTNDNH | 2.05 | 2.08 | 0.09 | 1.66 |
| ATLVT012XX1751 | 46685 | SSAAGAASNDNH | −2.69 | −3.06 | −3.22 | −3.6 | ATLVT012XX1797 | 46731 | SSASAGSTNDNT | 0.4 | 0.36 | 0.26 | 0.1 |
| ATLVT012XX1752 | 46686 | SSAAGAASNDNT | −2.47 | −1.71 | −0.84 | −3.67 | ATLVT012XX1798 | 46732 | SSASGAASNDNA | −1.74 | −1.84 | −1.9 | 1.14 |
| ATLVT012XX1753 | 46687 | SSAAGAATNDNA | −0.48 | −0.01 | −1.29 | −0.35 | ATLVT012XX1799 | 46733 | SSASGAASNDNH | −0.06 | −0.73 | −0.59 | 1.37 |
| ATLVT012XX1754 | 46688 | SSAAGAATNDNH | 1.48 | 1.83 | −0.63 | −0.15 | ATLVT012XX1800 | 46734 | SSASGAASNDNT | −1.66 | −1.86 | −0.36 | 0.33 |
| ATLVT012XX1755 | 46689 | SSAAGAATNDNT | −2.06 | −1.46 | −3 | −2.52 | ATLVT012XX1801 | 46735 | SSASGAATNDNA | −1.57 | 0.78 | −0.68 | 0.76 |
| ATLVT012XX1756 | 46690 | SSAAGASSNDNA | −1.3 | 0.33 | −2.31 | −1.02 | ATLVT012XX1802 | 46736 | SSASGAATNDNH | −0.19 | 1.55 | 0.14 | −1.65 |
| ATLVT012XX1757 | 46691 | SSAAGASSNDNH | −1.7 | −1.72 | −0.34 | −1.23 | ATLVT012XX1803 | 46737 | SSASGAATNDNT | −1.17 | 2.87 | 0.27 | −2.11 |
| ATLVT012XX1758 | 46692 | SSAAGASSNDNT | −2.1 | −1.71 | 0.36 | −1.4 | ATLVT012XX1804 | 46738 | SSASGASSNDNA | −0.25 | 0.08 | 0.55 | −1.13 |
| ATLVT012XX1759 | 46693 | SSAAGASTNDNA | −1.21 | −1.03 | −2.03 | −1.41 | ATLVT012XX1805 | 46739 | SSASGASSNDNH | 0.38 | −1.93 | −0.74 | −2.05 |
| ATLVT012XX1760 | 46694 | SSAAGASTNDNH | −1.82 | −1.95 | −0.51 | −1.09 | ATLVT012XX1806 | 46740 | SSASGASSNDNT | −0.13 | 0.85 | 0.19 | −1.12 |
| ATLVT012XX1761 | 46695 | SSAAGASTNDNT | 0.87 | −1.41 | −2.52 | −0.52 | ATLVT012XX1807 | 46741 | SSASGASTNDNA | −1.52 | −1.03 | 0.66 | 0.25 |
| ATLVT012XX1762 | 46696 | SSAAGGASNDNA | 0.29 | −0.17 | −2.33 | 0.77 | ATLVT012XX1808 | 46742 | SSASGASTNDNH | 0.12 | 0.18 | 1.4 | 0.18 |
| ATLVT012XX1763 | 46697 | SSAAGGASNDNH | 0.44 | 1.15 | −0.8 | 2.58 | ATLVT012XX1809 | 46743 | SSASGASTNDNT | 0.69 | 1.44 | 0.01 | 0.44 |
| ATLVT012XX1764 | 46698 | SSAAGGASNDNT | 0.01 | 1.04 | −0.44 | 0.94 | ATLVT012XX1810 | 46744 | SSASGGASNDNA | 1.33 | 1.25 | −0.12 | 0.95 |
| ATLVT012XX1765 | 46699 | SSAAGGATNDNA | −0.3 | 2.36 | −0.06 | 0.82 | ATLVT012XX1811 | 46745 | SSASGGASNDNH | 2.06 | 2.75 | 1.23 | 3.48 |
| ATLVT012XX1766 | 46700 | SSAAGGATNDNH | 6.01 | 6.5 | −0.29 | 4.3 | ATLVT012XX1812 | 46746 | SSASGGASNDNT | 1.49 | 2.89 | −0.36 | 0.55 |
| ATLVT012XX1767 | 46701 | SSAAGGATNDNT | 0.7 | 1.17 | 0.47 | 1.44 | ATLVT012XX1813 | 46747 | SSASGGATNDNA | 3.01 | 3.42 | 0.81 | 3.11 |
| ATLVT012XX1768 | 46702 | SSAAGGSSNDNA | 1.65 | 1.39 | −1.77 | −0.97 | ATLVT012XX1814 | 46748 | SSASGGATNDNH | 7.69 | 9.53 | 1.61 | 4.65 |
| ATLVT012XX1769 | 46703 | SSAAGGSSNDNH | −1.94 | −0.23 | −3.52 | −0.6 | ATLVT012XX1815 | 46749 | SSASGGATNDNT | 2.9 | 4.12 | −0.35 | 2.56 |
| ATLVT012XX1770 | 46704 | SSAAGGSSNDNT | 0.21 | 1.02 | −1.29 | 0.5 | ATTVT012XX1816 | 46750 | SSASGGSSNDNA | 2.71 | 2.52 | −0.04 | −0.47 |
| ATLVT012XX1771 | 46705 | SSAAGGSTNDNA | 0.74 | 2.05 | −0.75 | 1.48 | ATLVT012XX1817 | 46751 | SSASGGSSNDNH | 0.8 | 1.63 | 0.28 | 1.04 |
| ATLVT012XX1772 | 46706 | SSAAGGSTNDNH | −0.52 | 3.43 | −2.24 | 2.23 | ATLVT012XX1818 | 46752 | SSASGGSSNDNT | 2.7 | 3.32 | −0.71 | −0.35 |
| ATLVT012XX1773 | 46707 | SSAAGGSTNDNT | 0.5 | 1.21 | 0.06 | 1.24 | ATLVT012XX1819 | 46753 | SSASGGSTNDNA | 3.49 | 2.29 | 1.34 | 0.58 |
| ATLVT012XX1774 | 46708 | SSASAAASNDNA | −1.3 | 2.34 | 0.9 | 0.52 | ATLVT012XX1820 | 46754 | SSASGGSTNDNH | 3.94 | 3.67 | −0.12 | 1.67 |
| ATLVT012XX1775 | 46709 | SSASAAASNDNH | −1.48 | 1.85 | 0.3 | 1.36 | ATLVT012XX1821 | 46755 | SSASGGSTNDNT | 2.71 | 3.7 | 0.22 | 0.49 |
| ATLVT012XX1776 | 46710 | SSASAAASNDNT | −0.81 | 1.1 | 0.13 | 0.18 | ATLVT012XX1822 | 46756 | SSATAAASNDNA | −1.36 | 1.13 | −0.2 | 0.49 |
| ATLVT012XX1777 | 46711 | SSASAAANDNA | 1 | 2.2 | −0.4 | 1.06 | ATLVT012XX1823 | 46757 | SSATAAASNDNH | 0.82 | −1.46 | −0.93 | 0.68 |
| ATLVT012XX1778 | #6712 | SSASAAATNDNH | −1.05 | 4.38 | −0.09 | 1.61 | ATLVT012XX1824 | 46758 | SSATAAASNDNT | 0.85 | −1.09 | −0.54 | 1.67 |
| ATLVT01XXX1779 | 46713 | SSASAAATNDNT | 0.84 | 0.59 | 1.19 | −0.98 | ATLVT012XX1825 | 46759 | SSATAAATNDNA | −0.48 | 2.22 | 0.54 | 2.62 |
| ATLVT012XX1780 | 46714 | SSASAASSNDNA | 1.62 | 2.42 | 0.33 | −0.53 | ATLVT012XX1826 | 46760 | SSATAAATNDNH | 0.26 | 0.07 | 0.3 | 1.92 |
| ATLVT012XX1781 | 46715 | SSASAASSNDNH | 1.96 | 1.78 | 0.55 | 0.35 | ATLVT012XX1827 | 46761 | SSATAAATNDNT | 2.36 | 1.98 | 0.43 | 0.72 |
| ATLVT012XX1782 | 46716 | SSASAASSNDNT | 2.02 | 1.3 | 0.54 | −0.31 | ATLVT012XX1828 | 46762 | SSATAASSNDNA | 1.87 | −1.34 | −0.77 | −1.26 |
| ATLVT012XX1783 | 46717 | SSASAASTNDNA | 3.58 | 1.62 | −0.14 | −0.86 | ATLVT012XX1829 | 46763 | SSATAASSNDNH | 2.47 | −1.46 | 1.09 | −0.62 |
| ATLVT012XX1784 | 46718 | SSASAASTNDNH | 1.47 | 0.98 | −0.71 | −0.53 | ATLVT012XX1830 | 46764 | SSATAASSNDNT | 1.48 | 0.63 | −0.41 | 1.36 |
| ATLVT012XX1785 | 46719 | SSASAASTNDNT | 2.32 | 2.48 | 0.78 | −1.02 | ATLVT012XX1831 | 46765 | SSATAASTNDNA | 1.68 | 2.09 | 1.06 | 0.53 |
| ATLVT012XX1786 | 46720 | SSASAGASNDNA | 2.04 | 1.32 | −1.38 | 0.29 | ATLVT012XX1832 | 46766 | SSATAASTNDNH | 1.46 | 0.77 | 0.42 | 0.64 |
| ATLVT012XX1787 | 46721 | SSASAGASNDNH | 1.09 | 2.55 | −1.6 | −0.04 | ATLVT012XX1833 | 46767 | SSATAASTNDNT | 1.79 | 1.81 | 0.63 | 0.43 |
| ATLVT012XX1788 | 46722 | SSASAGASNDNT | 0.12 | −1.29 | 0.48 | 0.43 | ATLVT012XX1834 | 46768 | SSATAGASNDNA | −0.85 | 1.99 | 1.58 | −0.99 |
| ATLVT012XX1789 | 46723 | SSASAGATNDNA | −0.86 | −1.1 | −0.43 | −0.25 | ATLVT012XX1835 | 46769 | SSATAGASNDNH | 0.81 | 0 | −0.19 | 1.94 |
| ATLVT012XX1790 | 46724 | SSASAGATNDNH | 1.99 | 2.51 | 0.38 | 1.85 | ATLVT012XX1836 | 46770 | SSATAGASNQNT | 1.06 | 2.01 | 0.44 | 0.52 |
| ATLVT012XX1791 | 46725 | SSASAGATNDNT | 1.31 | 1 | −1.35 | 0.87 | ATLVT012XX1837 | 46771 | SSATAGATNDNA | 2.96 | 1.53 | −0.89 | 0.64 |
| ATLVT012XX1792 | 46726 | SSASAGSSNDNA | 1.82 | −1.58 | 0.77 | −0.81 | ATLVT012XX1838 | 46772 | SSATAGATNDNH | 1.68 | 0.48 | −0.03 | 0.85 |
| ATLVT012XX1793 | 46727 | SSASAGSSNDNH | 0.29 | −0.93 | 0.72 | −1.6 | ATLVT012XX1839 | 46773 | SSATAGATNDNT | 3.35 | 0.85 | −1.02 | 1.34 |
| ATLVT012XX1794 | 46728 | SSASAGSSNDNT | 0.05 | −1.13 | 0.74 | 0.01 | ATLVT012XX1840 | 46774 | SSATAGSSNDNA | 0.23 | −1.33 | 0.45 | −0.02 |
| ATLVT012XX1841 | 46775 | SSATAGSSNDNH | −0.56 | −1.34 | −0.47 | −1.54 | ATLVT012XX1887 | 46821 | SSEAAGATNDNT | −0.43 | 0.99 | −1.32 | −0.84 |
| ATLVT012XX1842 | 46776 | SSATAGSSNDNT | 1.25 | 1.65 | −0.1 | 0.86 | ATLVT012XX1888 | 46822 | SSEAAGSSNDNA | −0.18 | −1.89 | −0.21 | −1.92 |
| ATLVT012XX1843 | 46777 | SSATAGSTNDNA | 0.43 | 0.19 | 0.07 | −0.33 | ATLVT012XX1889 | 46823 | SSEAAGSSNDNH | −2.62 | −2.78 | −0.39 | −1.52 |
| ATLVT012XX1844 | 46778 | SSATAGSTNDNH | 1.19 | 1.4 | 0.64 | 0.16 | ATLVT012XX1890 | 46824 | SSEAAGSSNDNT | −1.98 | −2.52 | −0.32 | −3.61 |
| ATLVT012XX1845 | 46779 | SSATAGSTNDNT | 0.74 | 2.44 | −0.12 | 1.69 | ATLVT012XX1891 | 46825 | SSEAAGSTNDNA | −3.49 | −2.17 | −2.76 | −2.51 |
| ATLVT012XX1846 | 46780 | SSATGAASNDNA | 0.03 | −1.83 | −0.15 | −0.75 | ATLVT012XX1892 | 46826 | SSEAAGSTNDNH | −3.68 | −2.32 | −0.36 | −1.88 |
| ATLVT012XX1847 | 46781 | SSATGAASNDNH | 0.32 | −1.51 | 0.3 | 1.13 | ATLVT012XX1893 | 46827 | SSEAAGSTNDNT | −3.06 | −2.81 | −0.84 | −2.91 |
| ATLVT012XX1848 | 46782 | SSATGAASNDNT | 0.46 | −2.04 | 1.23 | 0.52 | ATLVT012XX1894 | 46828 | SSEAGAASNDNA | 0.53 | −0.93 | −0.76 | −1.11 |
| ATLVT012XX1849 | 46783 | SSATGAATNDNA | 0.07 | 0.73 | −1.14 | −0.06 | ATLVT012XX1895 | 46829 | SSEAGAASNDNH | −0.27 | −0.37 | −0.43 | −0.35 |
| ATLVT012XX1850 | 46784 | SSATGAATNDNH | 0.5 | 0.44 | −0.55 | 1.51 | ATLVT012XX1896 | 46830 | SSEAGAASNDNT | 0.08 | −1.35 | 0.38 | −0.77 |
| ATLVT012XX1851 | 46785 | SSATGAATNDNT | −0.7 | −1.43 | −0.08 | −1.7 | ATLVT012XX1897 | 46831 | SSEAGAATNDNA | −2.58 | −0.07 | −1.51 | −0.63 |
| ATLVT012XX1852 | 46786 | SSATGASSNDNA | 3.19 | −0.01 | 0.73 | −0.02 | ATLVT012XX1898 | 46832 | SSEAGAATNDNH | −0.2 | 3.91 | 0.43 | 1.62 |
| ATLVT012XX1853 | 46787 | SSATGASSNDNH | −0.12 | −1.55 | 0.66 | −1.46 | ATLVT012XX1899 | 46833 | SSEAGAATNDNT | −1.36 | 0.71 | 0.11 | 2.34 |
| ATLVT012XX1854 | 46788 | SSATGASSNDNT | 3.85 | −1.49 | −0.22 | 0.07 | ATLVT012XX1900 | 46834 | SSEAGASSNDNA | 0.72 | 1.65 | 0.49 | −1.03 |
| ATLVT012XX1855 | 46789 | SSATGASTNDNA | 4.52 | 2.82 | 0.82 | −0.37 | ATLVT012XX1901 | 46835 | SSEAGASSNDNH | 0.77 | −1.84 | 0.61 | −1.05 |
| ATLVT012XX1856 | 46790 | SSATGASTNDNH | 2.9 | 0.18 | 0.34 | 1.55 | ATLVT012XX1902 | 46836 | SSEAGASSNDNT | 0.41 | −1.3 | −0.03 | −1.47 |
| ATLVT012XX1857 | 46791 | SSATGASTNDNT | 2.3 | 1.37 | 0.99 | −1.06 | ATLVT012XX1903 | 46837 | SSEAGASTNDNA | −0.62 | −0.55 | −0.94 | −0.92 |
| ATLVT012XX1858 | 46792 | SSATGGASNDNA | −1.11 | 1.3 | 1.11 | 1.67 | ATLVT012XX1904 | 46838 | SSEAGASTNDNH | −2.46 | −0.04 | 1.43 | 0.57 |
| ATLVT012XX1859 | 46793 | SSATGGASNDNH | 1.58 | 2.55 | 0.8 | 2.6 | ATLVT012XX1905 | 46839 | SSEAGASTNDNT | −0.57 | 1.4 | −0.58 | 0.53 |
| ATLVT012XX1860 | 46794 | SSATGGASNDNT | 0.35 | 2.5 | 0.84 | 0.47 | ATLVT012XX1906 | 46840 | SSEAGGASNDNA | 2.21 | 2.49 | −0.9 | 2.23 |
| ATLVT012XX1861 | 46795 | SSATGGATNDNA | 3.46 | 2.67 | 0.14 | 1.32 | ATLVT012XX1907 | 46841 | SSEAGGASNDNH | 2.96 | 3.56 | 0.22 | 3.37 |
| ATLVT012XX1862 | 46796 | SSATGGATNDNH | 6.41 | 6.53 | 0.47 | 4.62 | ATLVT012XX1908 | 46842 | SSEAGGASNDNT | 1.9 | 3.04 | −0.15 | 1.55 |
| ATLVT012XX1863 | 46797 | SSATGGATNDNT | 2.07 | 3.64 | 0.87 | 2.07 | ATLVT012XX1909 | 46843 | SSEAGGATNDNA | 2.47 | 4.87 | 1.28 | 1.94 |
| ATLVT012XX1864 | 46798 | SSATGGSSNDNA | 0.97 | 1.97 | −0.06 | 0.87 | ATLVT012XX1910 | 46844 | SSEAGGATNDNH | 7.9 | 10.05 | 1.97 | 4.58 |
| ATLVT012XX1865 | 46799 | SSATGGSSNDNH | −0.45 | 0.69 | 0.83 | 0.66 | ATLVT012XX1911 | 46845 | SSEAGGATNDNT | 3.93 | 4.47 | 0.01 | 2.5 |
| ATLVT012XX1866 | 46800 | SSATGGSSNDNT | 1.26 | 1.75 | 0.18 | −0.42 | ATLVT012XX1912 | 46846 | SSEAGGSSNDNA | 1.17 | 3.02 | 0.01 | −0.04 |
| ATLVT012XX1867 | 46801 | SSATGGSTNDNA | 2.01 | 2.58 | 0.35 | 1.88 | ATLVT012XX1913 | 46847 | SSEAGGSSNDNH | 1.47 | 3.86 | 1.8 | 0.61 |
| ATLVT012XX1868 | 46802 | SSATGGSTNDNH | 3 | 2.89 | 0.04 | 2.15 | ATLVT012XX1914 | 46848 | SSEAGGSSNDNT | 1.92 | 3.33 | 0.18 | 0.7 |
| ATLVT012XX1869 | 46803 | SSATGGSTNDNT | 2.82 | 1.72 | −0.01 | 1.81 | ATLVT012XX1915 | 46849 | SSEAGGSTNDNA | 2.75 | 5.24 | 1.09 | 1.85 |
| ATLVT012XX1870 | 46804 | SSEAAAASNDNA | 2.64 | 0.46 | 0.56 | 0.16 | ATLVT012XX1916 | 46850 | SSEAGGSTNDNH | 0.59 | 2.26 | 0.39 | 2.58 |
| ATLVT012XX1871 | 46805 | SSEAAAASNDNH | 1.68 | 1.11 | −0.02 | −0.37 | ATLVT012XX1917 | 46851 | SSEAGGSTNDNT | 1.95 | 21 | 0.42 | 0.92 |
| ATLVT012XX1872 | 46806 | SSEAAAASNDNT | 0.24 | −0.01 | 0.55 | −0.09 | ATLVT012XX1918 | 46852 | SSESAAASNDNA | 0.94 | −1.56 | −0.33 | −1.6 |
| ATLVT012XX1873 | 46807 | SSEAAAATNDNA | 0.75 | 1.71 | −0.88 | −0.81 | ATLVT012XX1919 | 46853 | SSESAAASNDNH | −0.67 | −0.32 | −0.04 | −0.36 |
| ATLVT012XX1874 | 46808 | SSEAAAATNDNH | 0.34 | 0.8 | 0.08 | 0.1 | ATLVT012XX1920 | 46854 | SSESAAASNDNT | −1.65 | −3.13 | −0.14 | 1.59 |
| ATLVT012XX1875 | 46809 | SSEAAAATNDNT | 1.17 | −1.26 | 0.39 | 0.22 | ATLVT012XX1921 | 46855 | SSESAAATNDNA | −1.11 | 0.49 | 0.55 | −1.02 |
| ATLVT012XX1876 | 46810 | SSEAAASSNDNA | 0.23 | 2.35 | 0.55 | −0.23 | ATLVT012XX1922 | 46856 | SSESAAATNDNH | 2.55 | −0.01 | 0.78 | −1.75 |
| ATLVT012XX1877 | 46811 | SSEAAASSNDNH | 2.65 | 0.34 | −0.49 | −1.12 | ATLVT012XX1923 | 46857 | SSESAAATNDNT | −0.19 | 0.18 | 1.07 | −0.12 |
| ATLVT012XX1878 | 46812 | SSEAAASSNDNT | 2.07 | 2.13 | 1.16 | −1.15 | ATLVT012XX1924 | 46858 | SSESAASSNDNA | 0.67 | −0.9 | 0.97 | 0.11 |
| ATLVT012XX1879 | 46813 | SSEAAASTNDNA | −0.37 | 1.51 | 0.5 | −3.49 | ATLVT012XX1925 | 46859 | SSESAASSNDNH | −0.62 | 1.34 | 0.04 | −0.44 |
| ATLVT012XX1880 | 46814 | SSEAAASTNDNH | −0.19 | −0.57 | −0.28 | −0.55 | ATLVT012XX1926 | 46860 | SSESAASSNDNT | 1.34 | 2.7 | 0.02 | −2.14 |
| ATLVT012XX1881 | 46815 | SSEAAASTNDNT | 1.95 | 0.14 | 0.72 | −2.59 | ATLVT012XX1927 | 46861 | SSESAASTNDNA | 2.55 | −1.23 | −0.54 | −0.53 |
| ATLVT012XX1882 | 46816 | SSEAAGASNDNA | −2.76 | −0.36 | −1.49 | −3.14 | ATLVT012XX1928 | 46862 | SSESAASTNDNH | 1.17 | −1.19 | −1.05 | −1.12 |
| ATLVT012XX1883 | 46817 | SSEAAGASNDNH | −1.13 | −0.38 | −0.3 | −0.74 | ATLVT012XX1929 | 46863 | SSESAASTNDNT | 0.56 | −2.16 | 0.66 | −1.19 |
| ATLVT012XX1884 | 46818 | SSEAAGASNDNT | −2.34 | −0.94 | −1.45 | −1.68 | ATLVT012XX1930 | 46864 | SSESAGASNDNA | 0.66 | −0.16 | −0.92 | 0.79 |
| ATLVT012XX1885 | 46819 | SSEAAGATNDNA | 2.53 | −1.16 | −2.26 | −0.69 | ATLVT012XX1931 | 46865 | SSESAGASNDNH | 0.3 | 1.33 | −0.93 | −1.74 |
| ATLVT012XX1886 | 46820 | SSEAAGATNDNH | −0.99 | 1.14 | −1.41 | −0.03 | ATLVT012XX1932 | 46866 | SSESAGASNDNT | −1.35 | −1.39 | 0.58 | −2.33 |
| ATLVT012XX1933 | 46867 | SSESAGATNDNA | −2.56 | 0.16 | −0.73 | −0.78 | ATLVT012XX1979 | 46913 | SSETAGASNDNH | 0.8 | −1.69 | −0.98 | 0.81 |
| ATLVT012XX1934 | 46868 | SSESAGATNDNH | −1.05 | −0.39 | −0.18 | 0.28 | ATLVT012XX1980 | 46914 | SSETAGASNDNT | −0.26 | 1.12 | −1.29 | 0.36 |
| ATLVT012XX1935 | 46869 | SSESAGATNDNT | −2.53 | −1.16 | −0.98 | −2.42 | ATLVT012XX1981 | 46915 | SSETAGATNDNA | −1.08 | 1.21 | −1.55 | −1.82 |
| ATLVT012XX1936 | 46870 | SSESAGSSNDNA | 0.37 | −1.46 | 0.31 | −2.25 | ATLVT012XX1982 | 46916 | SSETAGATNDNH | 0.31 | 1.69 | −2.17 | 1.1 |
| ATLVT012XX1937 | 46871 | SSESAGSSNDNH | 0.1 | −1.51 | 0.59 | −2.13 | ATLVT012XX1983 | 46917 | SSETAGATNDNT | −1.86 | 0.61 | −0.16 | −1.56 |
| ATLVT012XX1938 | 46872 | SSESAGSSNDNT | 0.56 | −1.54 | 0.58 | −3.08 | ATLVT012XX1984 | 46918 | SSETAGSSNDNA | 3.06 | −1.89 | −0.3 | 1.42 |
| ATLVT012XX1939 | 46873 | SSESAGSTNDNA | 0.33 | 3.62 | −0.49 | −0.54 | ATLVT012XX1985 | 46919 | SSETAGSSNDNH | −0.02 | −0.5 | 1.31 | 1.44 |
| ATLVT012XX1940 | 46874 | SSESAGSTNDNH | 1.76 | 1.99 | 0.14 | −1.8 | ATLVT012XX1986 | 46920 | SSETAGSSNDNT | 1.82 | −2.53 | 0.27 | −0.42 |
| ATLVT012XX1941 | 46875 | SSESAGSTNDNT | −0.58 | 1.18 | −0.1 | −1.19 | ATLVT012XX1987 | 46921 | SSETAGSTNDNA | −0.77 | −1.1 | −0.7 | −0.98 |
| ATLVT012XX1942 | 46876 | SSESGAASNDNA | −2.55 | −2.36 | −1.77 | −1.44 | ATLVT012XX1988 | 46922 | SSETAGSTNDNH | 0.09 | −2.44 | −0.58 | −0.48 |
| ATLVT012XX1943 | 46877 | SSESGAASNDNH | −1.24 | −1.99 | −2.18 | −1.57 | ATLVT012XX1989 | 46923 | SSETAGSTNDNT | −1.17 | −2.03 | −0.49 | −0.05 |
| ATLVT012XX1944 | 46878 | SSESGAASNDNT | −0.84 | −2.66 | −3.32 | −2.32 | ATLVT012XX1990 | 46924 | SSETGAASNDNA | −3.15 | −1.1 | −1.02 | −3.12 |
| ATLVT012XX1945 | 46879 | SSESGAATNDNA | −0.58 | −0.36 | 1.16 | 0.8 | ATLVT012XX1991 | 46925 | SSETGAASNDNH | −3.01 | −1.74 | 1.81 | −0.58 |
| ATLVT012XX1946 | 46880 | SSESGAATNDNH | 0.03 | −0.33 | −1.5 | −0.41 | ATLVT012XX1992 | 46926 | SSETGAASNDNT | −2.61 | −1.31 | −0.4 | −2.87 |
| ATLVT012XX1947 | 46881 | SSESGAATNDNT | −1.9 | −0.99 | −0.5 | 0.34 | ATLVT012XX1993 | 46927 | SSETGAATNDNA | −2.23 | 0.12 | −1.89 | −2.09 |
| ATLVT012XX1948 | 46882 | SSESGASSNDNA | −0.08 | −0.18 | 1.27 | 0.77 | ATLVT012XX1994 | 46928 | SSETGAATNDNH | 0.76 | 1.02 | −0.55 | −2.17 |
| ATLVT012XX1949 | 46883 | SSESGASSNDNH | −2.77 | −2.82 | −0.86 | 0.18 | ATTVT012XX1995 | 46929 | SSETGAATNDNT | −1.66 | −0.49 | 0.23 | −3.72 |
| ATLVT012XX1950 | 46884 | SSESGASSNDNT | −1.73 | −0.41 | −1.07 | −0.52 | ATLVT012XX1996 | 46930 | SSETGASSNDNA | −2.31 | 1.87 | 0.39 | −0.6 |
| ATLVT012XX1951 | 46885 | SSESGASTNDNA | −1.26 | −2.55 | 0.71 | −2.12 | ATLVT012XX1997 | 46931 | SSETGASSNDNH | −1.76 | 0.43 | −0.26 | −0.11 |
| ATLVT012XX1952 | 46886 | SSESGASTNDNH | −1.92 | −2.81 | 0.32 | −1.54 | ATLVT012XX1998 | 46932 | SSETGASSNDNT | −2.81 | −0.21 | −0.26 | 1 |
| ATLVT012XX1953 | 46887 | SSESGASTNDNT | −0.09 | −1.91 | −0.19 | −1.6 | ATLVT012XX1999 | 46933 | SSETGASTNDNA | 0.04 | 0.99 | 1.35 | −2.23 |
| ATLVT012XX1954 | 46888 | SSESGGASNDNA | 2.15 | 3.23 | 0.21 | 1.07 | ATLVT012XX2000 | 46934 | SSETGASTNDNH | −2.13 | −0.32 | 0.21 | 0.17 |
| ATLVT012XX1955 | 46889 | SSESGGASNDNH | 2.63 | 5.06 | 1.27 | 1.65 | ATLVT012XX2001 | 46935 | SSETGASTNDNT | −0.33 | −1.24 | −0.18 | −1.25 |
| ATLVT012XX1956 | 46890 | SSESGGASNDNT | 1.9 | 3.12 | −0.12 | 0.19 | ATLVT012XX2002 | 46936 | SSETGGASNDNA | 2.94 | 2.11 | 0.06 | 0.66 |
| ATLVT012XX1957 | 46891 | SSESGGATNDNA | 2.71 | 2.5 | 0.49 | 1.71 | ATLVT012XX2003 | 46937 | SSETGGASNDNH | 2.21 | 1.44 | 0.75 | 1.5 |
| ATLVT012XX1958 | 46892 | SSESGGATNDNH | 7.18 | 8.78 | 1.5 | 3.92 | ATLVT012XX2004 | 46938 | SSETGGASNDNT | 3.71 | 0.43 | 1.06 | −0.13 |
| ATLVT012XX1959 | 46893 | SSESGGATNDNT | 2.46 | 2.7 | 0.26 | 2.23 | ATLVT012XX2005 | 46939 | SSETGGATNDNA | 3.47 | 1.69 | 0.06 | 2.94 |
| ATLVT012XX1960 | 46894 | SSESGGSSNDNA | 1.02 | 0.46 | 0.15 | −0.17 | ATLVT012XX2006 | 46940 | SSETGGATNDNH | 7.01 | 6.38 | 1.31 | 3.98 |
| ATLVT012XX1961 | 46895 | SSESGGSSNDNH | 0.67 | 1.3 | 0.48 | 0.55 | ATLVT012XX2007 | 46941 | SSETGGATNDNT | 3.42 | 0.27 | 0.13 | 1.24 |
| ATLVT012XX1962 | 46896 | SSESGGSSNDNT | 2.06 | 0.48 | −0.67 | −1.69 | ATLVT012XX2008 | 46942 | SSETGGSSNDNA | 1.17 | −0.05 | 0.36 | 0.16 |
| ATLVT012XX1963 | 46897 | SSESGGSTNDNA | 2.37 | 0.94 | 0.13 | 0.27 | ATLVT012XX2009 | 46943 | SSETGGSSNDNH | 1.35 | −1.77 | 0.67 | 0.82 |
| ATLVT012XX1964 | 46898 | SSESGGSTNDNH | 1.25 | 0.84 | 0.09 | 1.24 | ATLVT012XX2010 | 46944 | SSETGGSSNDNT | 1.91 | −0.64 | 0.3 | 0.57 |
| ATLVT012XX1965 | 46899 | SSESGGSTNDNT | 0.88 | 0.82 | 0.86 | 0.6 | ATLVT012XX2011 | 46945 | SSETGGSTNDNA | 0.96 | 0.53 | 0.7 | 1.26 |
| ATLVT012XX1966 | 46900 | SSETAAASNDNA | −0.85 | −1.51 | 0.36 | −1.62 | ATLVT012XX2012 | 46946 | SSETGGSTNDNH | 1.02 | 0.37 | 0.86 | 2.46 |
| ATLVT012XX1967 | 46901 | SSETAAASNDNH | −2.47 | 2.08 | −0.01 | −2.47 | ATLVT012XX2013 | 46947 | SSETGGSTNDNT | 2.56 | 0.66 | −0.31 | 0.43 |
| ATLVT012XX1968 | 46902 | SSETAAASNDNT | −1.81 | −2.12 | −0.16 | −2.13 | ATLVT012XX2014 | 46948 | SSQAAAASNDNA | −2.19 | −0.45 | −1.58 | −0.18 |
| ATLVT012XX1969 | 46903 | SSETAAATNDNA | 0.18 | −0.57 | −1.47 | 1.71 | ATLVT012XX2015 | 46949 | SSQAAAASNDNH | −0.31 | −0.1 | 0.64 | 0.29 |
| ATLVT012XX1970 | 46904 | SSETAAATNDNH | −0.88 | −1.01 | −1.88 | −0.44 | ATTVT012XX2016 | 46950 | SSQAAAASNDNT | −1.44 | 1.83 | 0.4 | 1 |
| ATLVT012XX1971 | 46905 | SSETAAATNDNT | 2.88 | 0.99 | −1.09 | 0.16 | ATLVT012XX2017 | 46951 | SSQAAAATNDNA | 2 | −0.45 | 1.53 | 2.53 |
| ATLVT012XX1972 | 46906 | SSETAASSNDNA | 1.96 | −1.46 | 0.09 | −1.49 | ATLVT012XX2018 | 46952 | SSQAAAATNDNH | 0.37 | −0.18 | 0.46 | 1.53 |
| ATLVT012XX1973 | 46907 | SSETAASSNDNH | −1.67 | −2.87 | −0.13 | −1.22 | ATLVT012XX2019 | 46953 | SSQAAAATNDNT | 1.03 | 1.63 | −0.42 | 0.99 |
| ATLVT012XX1974 | 46908 | SSETAASSNDNT | 0.3 | −1.99 | −1.36 | −0.69 | ATLVT012XX2020 | 46954 | SSQAAASSNDNA | −0.9 | −1.4 | 0.8 | 0.82 |
| ATLVT012XX1975 | 46909 | SSETAASTNDNA | 0.29 | 1.41 | 0.14 | 0.43 | ATLVT012XX2021 | 46955 | SSQAAASSNDNH | −1.49 | 1.26 | 0.35 | −0.21 |
| ATLVT012XX1976 | 46910 | SSETAASTNDNH | −1.6 | −0.89 | 0.11 | −0.1 | ATLVT012XX2022 | 46956 | SSQAAASSNDNT | 0.39 | −1.09 | 0.59 | −1.36 |
| ATLVT012XX1977 | 46911 | SSETAASTNDNT | −0.04 | −1.82 | 0.36 | −1.93 | ATLVT012XX2023 | 46957 | SSQAAASTNDNA | 1.85 | 1.74 | −0.08 | −0.49 |
| ATLVT012XX1978 | 46912 | SSETAGASNDNA | −0.52 | 1.8 | −0.77 | −0.64 | ATLVT012XX2024 | 46958 | SSQAAASTNDNH | 0.03 | −1.1 | 0.36 | 1.17 |
| ATLVT012XX2025 | 46959 | SSQAAASTNDNT | 1.58 | −0.14 | −0.48 | −1.64 | ATLVT012XX2071 | 47005 | SSQSAASTNDNA | −0.64 | −0.53 | −0.35 | 0.85 |
| ATLVT012XX2026 | 46960 | SSQAAGASNDNA | −0.27 | 1.99 | −2.34 | −1.12 | ATLVT012XX2072 | 47006 | SSQSAASTNDNH | −2.24 | 2.23 | 0.18 | −0.93 |
| ATLVT012XX2027 | 46961 | SSQAAGASNDNH | −0.38 | 0.67 | −0.64 | −0.98 | ATLVT012XX2073 | 47007 | SSQSAASTNDNT | −1.05 | 0.04 | −0.14 | 0.32 |
| ATLVT012XX2028 | 46962 | SSQAAGASNDNT | 1.36 | 1.82 | −0.61 | −0.51 | ATLVT012XX2074 | 47008 | SSQSAGASNDNA | −0.73 | 1.09 | 0.66 | 0.39 |
| ATLVT012XX2029 | 46963 | SSQAAGATNDNA | −1.19 | 1.92 | −0.29 | −0.85 | ATLVT012XX2075 | 47009 | SSQSAGASNDNH | −0.25 | 2.59 | −0.75 | 2.14 |
| ATLVT012XX2030 | 46964 | SSQAAGATNDNH | 1.35 | 2.48 | −1.3 | 3.58 | ATLVT012XX2076 | 47010 | SSQSAGASNDNT | −0.96 | −0.3 | −0.58 | −0.43 |
| ATLVT012XX2031 | 46965 | SSQAAGATNDNT | 0.04 | 0.03 | −2.72 | 0.93 | ATLVT012XX2077 | 47011 | SSQSAGATNDNA | −0.37 | 1.35 | 0.28 | 0.5 |
| ATLVT012XX2032 | 46966 | SSQAAGSSNDNA | −2.2 | 3.4 | −1.17 | −1.38 | ATLVT012XX2078 | 47012 | SSQSAGATNDNH | 1.43 | 3.96 | −0.03 | 2.21 |
| ATLVT012XX2033 | 46967 | SSQAAGSSNDNH | −1.9 | 0.16 | −1.73 | −0.64 | ATLVT012XX2079 | 47013 | SSQSAGATNDNT | 1.35 | 2.65 | −0.33 | 0.54 |
| ATLVT012XX2034 | 46968 | SSQAAGSSNDNT | −0.47 | 1.2 | −0.76 | 0.96 | ATLVT012XX2080 | 47014 | SSQSAGSSNDNA | −1.91 | 0.17 | −0.7 | −0.34 |
| ATLVT012XX2035 | 46969 | SSQAAGSTNDNA | 1.26 | 0.03 | −1.33 | 0.25 | ATLVT012XX2081 | 47015 | SSQSAGSSNDNH | −1.26 | −0.47 | −0.32 | −0.3 |
| ATLVT012XX2036 | 46970 | SSQAAGSTNDNH | 0.72 | 0.71 | −1.79 | 1.72 | ATLVT012XX2082 | 47016 | SSQSAGSSNDNT | −0.32 | 0.3 | −0.96 | −1.3 |
| ATLVT012XX2037 | 46971 | SSQAAGSTNQNT | 1.69 | 0.73 | −0.23 | 0.48 | ATLVT012XX2083 | 47017 | SSQSAGSTNDNA | −1.14 | 3.61 | 0.9 | 0.38 |
| ATLVT012XX2038 | 46972 | SSQAGAASNDNA | 1.73 | −0.17 | −1.96 | −1.52 | ATLVT012XX2084 | 47018 | SSQSAGSTNDNH | 0.2 | 0.92 | 0.09 | 2.05 |
| ATLVT012XX2039 | 46973 | SSQAGAASNDNH | 0.81 | −0.81 | 0.86 | 0.97 | ATLVT012XX2085 | 47019 | SSQSAGSTNDNT | −0.94 | 1.37 | 0.62 | 0.02 |
| ATLVT012XX2040 | 46974 | SSQAGAASNDNT | 1.67 | −2.32 | −0.42 | −0.59 | ATLVT012XX2086 | 47020 | SSQSGAASNDNA | 0.36 | 0.97 | 0.22 | −0.42 |
| ATLVT012XX2041 | 46975 | SSQAGAATNDNA | 1.11 | 2.96 | −0.71 | 2.13 | ATLVT012XX2087 | 47021 | SSQSGAASNDNH | −1.2 | 1.8 | −1.26 | −0.49 |
| ATLVT012XX2042 | 46976 | SSQAGAATNDNH | 0.86 | 2.54 | −1.03 | 1.37 | ATLVT012XX2088 | 47022 | SSQSGAASNDNT | 0.46 | 2.93 | 1.69 | −1.9 |
| ATLVT012XX2043 | 46977 | SSQAGAATNDNT | 0.45 | −0.63 | −0.12 | 1.02 | ATLVT012XX2089 | 47023 | SSQSGAATNDNA | −0.43 | 1.45 | 0.19 | 0.46 |
| ATLVT012XX2044 | 46978 | SSQAGASSNDNA | 2.63 | −0.14 | 0.03 | −0.04 | ATLVT012XX2090 | 47024 | SSQSGAATNDNH | −0.37 | 1.85 | −0.98 | 1.52 |
| ATLVT012XX2045 | 46979 | SSQAGASSNDNH | 1.36 | 0.62 | 1.2 | −0.68 | ATLVT012XX2091 | 47025 | SSQSGAATNDNT | −0.04 | 2.45 | −1.07 | 0.06 |
| ATLVT012XX2046 | 46980 | SSQAGASSNDNT | 0.31 | 0.13 | −0.48 | 0.47 | ATLVT012XX2092 | 47026 | SSQSGASSNDNA | −0.76 | 0.74 | −0.05 | −0.93 |
| ATLVT01XXX2047 | 46981 | SSQAGASTNDNA | 0.21 | 0.39 | 0.77 | 0.77 | ATLVT012XX2093 | 47027 | SSQSGASSNDNH | −0.06 | −1.08 | 0.17 | 0.48 |
| ATLVT012XX2048 | 46982 | SSQAGASTNDNH | 0.78 | 3.76 | 1.05 | 1.82 | ATLVT012XX2094 | 47028 | SSQSGASSNDNT | 1.44 | −0.52 | 0.64 | −1.02 |
| ATLVT012XX2049 | 46983 | SSQAGASTNDNT | 1.93 | 1.59 | 0.02 | −0.03 | ATLVT012XX2095 | 47029 | SSQSGASTNDNA | −0.11 | −0.53 | 0.12 | 1.67 |
| ATLVT012XX2050 | 46984 | SSQAGGASNDNA | 3 | 4.38 | −0.05 | 1.86 | ATLVT012XX2096 | 47030 | SSQSGASTNDNH | 0.5 | 0.06 | 0.7 | 2.44 |
| ATLVT012XX2051 | 46985 | SSQAGGASNDNH | 6.93 | 9.18 | 1.19 | 5.18 | ATLVT012XX2097 | 47031 | SSQSGASTNDNT | 1.24 | −0.34 | −0.1 | 1.16 |
| ATLVT012XX2052 | 46986 | SSQAGGASNDNT | 2.97 | 3.59 | 1.42 | 2.28 | ATLVT012XX2098 | 47032 | SSQSGGASNDNA | 2.71 | 3.44 | 0.16 | 1.45 |
| ATLVT012XX2053 | 46987 | SSQAGGATNDNA | 5.13 | 5.34 | −0.33 | 2.01 | ATLVT012XX2099 | 47033 | SSQSGGASNDNH | 6.62 | 8.06 | 1.73 | 4.09 |
| ATLVT012XX2054 | 46988 | SSQAGGATNDNH | 9.66 | 12.66 | −0.11 | 4.16 | ATTVT012XX2100 | 47034 | SSQSGGASNDNT | 4.3 | 5.75 | 0.08 | 2.1 |
| ATLVT012XX2055 | 46989 | SSQAGGATNDNT | 5.49 | 6.48 | 0.44 | 2.79 | ATLVT012XX2101 | 47035 | SSQSGGATNDNA | 4.56 | 5.51 | 0.97 | 2.28 |
| ATLVT012XX2056 | 46990 | SSQAGGSSNDNA | 2.75 | 4.23 | 0.16 | 1.03 | ATLVT012XX2102 | 47036 | SSQSGGATNDNH | 9.74 | 12.42 | 1.75 | 4.5 |
| ATLVT012XX2057 | 46991 | SSQAGGSSNDNH | 1.25 | 3.22 | 0.91 | 2.46 | ATLVT012XX2103 | 47037 | SSQSGGATNDNT | 5.48 | 5.42 | 0.15 | 2.9 |
| ATLVT012XX2058 | 46992 | SSQAGGSSNDNT | 2.49 | 4.42 | 0.08 | 1.53 | ATLVT012XX2104 | 47038 | SSQSGGSSNDNA | 3 | 2.54 | 0.47 | 2.2 |
| ATLVT012XX2059 | 46993 | SSQAGGSTNDNA | 3.72 | 4.8 | 0.74 | 3.08 | ATLVT012XX2105 | 47039 | SSQSGGSSNDNH | 1.62 | 1.58 | 0.76 | 2.3 |
| ATLVT012XX2060 | 46994 | SSQAGGSTNDNH | 7.65 | 8.13 | 1.37 | 3.86 | ATLVT012XX2106 | 47040 | SSQSGGSSNDNT | 2.88 | 3.03 | 0.51 | 0.39 |
| ATLVT012XX2061 | 46995 | SSQAGGSTNDNT | 3.71 | 4.53 | 0.88 | 2.41 | ATLVT012XX2107 | 47041 | SSQSGGSTNDNA | 5 | 6.16 | 0.95 | 1.56 |
| ATLVT012XX2062 | 46996 | SSQSAAASNDNA | 0.86 | 3 | 1.99 | −0.63 | ATLVT012XX2108 | 47042 | SSQSGGSTNDNH | 6.51 | 8.58 | 1.54 | 3.74 |
| ATLVT012XX2063 | 46997 | SSQSAAASNDNH | −0.98 | 1.53 | −1.1 | 0.06 | ATLVT012XX2109 | 47043 | SSQSGGSTNDNT | 4.49 | 5.72 | 1.51 | 3.29 |
| ATLVT012XX2064 | 46998 | SSQSAAASNDNT | −0.07 | −0.57 | −0.48 | 0.46 | ATLVT012XX2110 | 47044 | SSQTAAASNDNA | −0.69 | −0.85 | 0.23 | 0.33 |
| ATLVT012XX2065 | 46999 | SSQSAAATNDNA | −0.28 | 1.35 | 0.25 | −0.45 | ATLVT012XX2111 | 47045 | SSQTAAASNDNH | −0.07 | 2.89 | 0.26 | −1 |
| ATLVT012XX2066 | 47000 | SSQSAAATNDNH | −1.72 | 2.4 | 0.6 | 0.07 | ATLVT012XX2112 | 47046 | SSQTAAASNDNT | 0.4 | 0.88 | 0.04 | 0.73 |
| ATLVT012XX2067 | 47001 | SSQSAAATNDNT | −0.65 | 1.52 | 0.64 | 1.28 | ATLVT012XX2113 | 47047 | SSQTAAATNDNA | 2.07 | −0.21 | 0.75 | −0.42 |
| ATLVT012XX2068 | 47002 | SSQSAASSNDNA | 2.03 | 1.62 | 0.89 | −1.01 | ATLVT012XX2114 | 47048 | SSQTAAATNDNH | 0.63 | 2.98 | 0.24 | 0.6 |
| ATLVT012XX2069 | 47003 | SSQSAASSNDNH | −1.25 | 1.47 | −0.78 | −1.78 | ATLVT012XX2115 | 47049 | SSQTAAATNDNT | −0.56 | 1.35 | 1.16 | 0.23 |
| ATLVT012XX2070 | 47004 | SSQSAASSNDNT | 1.04 | 1.27 | 0.24 | −0.6 | ATLVT012XX2116 | 47050 | SSQTAASSNDNA | 0.78 | −0.54 | 1.03 | 0.38 |
| ATLVT012XX2117 | 47051 | SSQTAASSNDNH | −0.06 | 1 | −0.57 | −1.22 | ATLVT012XX2163 | 47097 | SSTAAAATNDNT | −0.99 | −1.03 | 0.96 | 0.88 |
| ATLVT01XXX2118 | 47052 | SSQTAASSNDNT | −1.17 | 1.26 | −0.47 | −1.47 | ATLVT012XX2164 | 47098 | SSTAAASSNDNA | −0.67 | 1.44 | 0.02 | −0.08 |
| ATLVT012XX2119 | 47053 | SSQTAASTNDNA | 1.87 | 0.02 | 0.67 | −0.05 | ATLVT012XX2165 | 47099 | SSTAAASSNDNH | −1.31 | 0.2 | 1.05 | 1.16 |
| ATLVT012XX2120 | 47054 | SSQTAASTNDNH | 3.35 | 1.83 | 1.19 | −0.52 | ATLVT012XX2166 | 47100 | SSTAAASSNDNT | 1.42 | 1.25 | 0.37 | 0.23 |
| ATLVT012XX2121 | 47055 | SSQTAASTNDNT | 0.23 | −0.99 | 0.37 | 1.66 | ATLVT012XX2167 | 47101 | SSTAAASTNDNA | 0.84 | 0.58 | −0.87 | −0.54 |
| ATLVT012XX2122 | 47056 | SSQTAGASNDNA | 4.17 | −0.39 | 1.07 | 0.99 | ATLVT012XX2168 | 47102 | SSTAAASTNDNH | 0.4 | 3.8 | −0.02 | −0.8 |
| ATLVT012XX2123 | 47057 | SSQTAGASNDNH | 0.86 | 1.04 | 0.39 | 0.97 | ATLVT012XX2169 | 47103 | SSTAAASTNDNT | 1.81 | −0.8 | −1.01 | 0.37 |
| ATLVT012XX2124 | 47058 | SSQTAGASNDNT | 1.61 | −0.54 | 0.57 | 1.08 | ATLVT012XX2170 | 47104 | SSTAAGASNDNA | −0.15 | 0.28 | −0.28 | −2.21 |
| ATLVT012XX2125 | 47059 | SSQTAGATNDNA | 1.09 | −0.72 | −0.27 | 0.45 | ATLVT012XX2171 | 47105 | SSTAAGASNDNH | 1.56 | 1.53 | −0.81 | 0 |
| ATLVT012XX2126 | 47060 | SSQTAGATNDNH | 2.46 | 1.42 | −0.01 | 4.17 | ATLVT012XX2172 | 47106 | SSTAAGASNDNT | 1.54 | 1.56 | −1.19 | −2.02 |
| ATLVT012XX2127 | 47061 | SSQTAGATNDNT | 0.69 | −1.01 | −0.75 | 1.32 | ATLVT012XX2173 | 47107 | SSTAAGATNDNA | 2.08 | 0.67 | 0.25 | 0.27 |
| ATLVT012XX2128 | 47062 | SSQTAGSSNDNA | −0.77 | −0.87 | −1.22 | 0.3 | ATLVT012XX2174 | 47108 | SSTAAGATNDNH | 2.73 | 1.85 | 0.58 | 3.64 |
| ATLVT012XX2129 | 47063 | SSQTAGSSNDNH | −0.3 | 2.96 | 0.57 | 1.72 | ATLVT012XX2175 | 47109 | SSTAAGATNDNT | 1.76 | 2.34 | −0.1 | −0.4 |
| ATLVT012XX2130 | 47064 | SSQTAGSSNDNT | 2.81 | 0.51 | 0.38 | 1.1 | ATLVT012XX2176 | 47110 | SSTAAGSSNDNA | 3.17 | −0.84 | 0.14 | 0.42 |
| ATLVT012XX2131 | 47065 | SSQTAGSTNDNA | 0.33 | 0.82 | 0.62 | 1.37 | ATLVT012XX2177 | 47111 | SSTAAGSSNDNH | 1.5 | −0.71 | −0.23 | −0.42 |
| ATLVT012XX2132 | 47066 | SSQTAGSTNDNH | 0.07 | −0.54 | 0.87 | 1.17 | ATLVT012XX2178 | 47112 | SSTAAGSSNDNT | 2.21 | 2.62 | −0.43 | 0.43 |
| ATLVT012XX2133 | 47067 | SSQTAGSTNDNT | 0.45 | 1.7 | 0.3 | 0.22 | ATLVT012XX2179 | 47113 | SSTAAGSTNDNA | 0.76 | −0.28 | 0.36 | −0.93 |
| ATLVT012XX2134 | 47068 | SSQTGAASNDNA | 1.61 | −0.4 | −0.99 | −1.67 | ATLVT012XX2180 | 47114 | SSTAAGSTNDNH | 0.21 | 1.31 | 1.13 | 0.26 |
| ATLVT012XX2135 | 47069 | SSQTGAASNDNH | −0.59 | −0.67 | −1.24 | −2.56 | ATLVT012XX2181 | 47115 | SSTAAGSTNDNT | 1.22 | 0.16 | −1.8 | 0.84 |
| ATLVT012XX2136 | 47070 | SSQTGAASNDNT | −0.32 | −1.03 | −0.3 | −3.3 | ATLVT012XX2182 | 47116 | SSTAGAASNDNA | 1.37 | 0.03 | −0.5 | −1.66 |
| ATLVT012XX2137 | 47071 | SSQTGAATNDNA | −0.84 | 2.38 | 0.98 | −2.29 | ATLVT012XX2183 | 47117 | SSTAGAASNDNH | 0.96 | 1.11 | −1.79 | 1.15 |
| ATLVT012XX2138 | 47072 | SSQTGAATNDNH | 3.09 | 1.73 | 1.48 | −1.05 | ATLVT012XX2184 | 47118 | SSTAGAASNDNT | −0.63 | −1.2 | 0.28 | −2.51 |
| ATLVT012XX2139 | 47073 | SSQTGAATNDNT | −0.99 | −0.17 | 0.88 | −1.35 | ATLVT012XX2185 | 47119 | SSTAGAATNDNA | 1.85 | 3.74 | −0.5 | 1.02 |
| ATLVT012XX2140 | 47074 | SSQTGASSNDNA | 2.1 | −0.28 | −0.75 | −1.1 | ATLVT012XX2186 | 47120 | SSTAGAATNDNH | 2.26 | 4 | 0.33 | 3.72 |
| ATLVT012XX2141 | 47075 | SSQTGASSNDNH | 2.67 | 1.86 | 0.26 | 0.09 | ATLVT012XX2187 | 47121 | SSTAGAATNDNT | 0.61 | 2.05 | −1.39 | 2.38 |
| ATLVT012XX2142 | 47076 | SSQTGASSNDNT | 1.88 | 2.12 | −1.01 | −2.1 | ATLVT012XX2188 | 47122 | SSTAGASSNDNA | −0.53 | 3.78 | −0.38 | −0.29 |
| ATLVT012XX2143 | 47077 | SSQTGASTNDNA | 1.81 | 1.56 | 0.15 | 1.26 | ATLVT012XX2189 | 47123 | SSTAGASSNDNH | −0.26 | −0.83 | −0.35 | 0.24 |
| ATLVT012XX2144 | 47078 | SSQTGASTNDNH | 0.93 | 2.38 | −0.28 | 2.31 | ATLVT012XX2190 | 47124 | SSTAGASSNDNT | 0.27 | 1.03 | 0.11 | −0.97 |
| ATLVT012XX2145 | 47079 | SSQTGASTNDNT | −0.18 | −0.03 | 0.57 | −0.45 | ATLVT012XX2191 | 47125 | SSTAGASTNDNA | −0.35 | 1.04 | −0.69 | 1.48 |
| ATLVT012XX2146 | 47080 | SSQTGGASNDNA | 2.82 | 3.36 | 0.67 | 2.89 | ATLVT012XX2192 | 47126 | SSTAGASTNDNH | −0.15 | 2.63 | 0.94 | 1.62 |
| ATLVT012XX2147 | 47081 | SSQTGGASNDNH | 5.66 | 7.47 | 1.57 | 3.99 | ATLVT012XX2193 | 47127 | SSTAGASTNDNT | 0.33 | −0.04 | 0.21 | 1.17 |
| ATLVT012XX2148 | 47082 | SSQTGGASNDNT | 3.13 | 2.58 | 1.31 | 0.88 | ATLVT012XX2194 | 47128 | SSTAGGASNDNA | 6.46 | 7.2 | 0.11 | 3.67 |
| ATLVT012XX2149 | 47083 | SSQTGGATNDNA | 4.06 | 3.93 | 0.11 | 2.66 | ATLVT012XX2195 | 47129 | SSTAGGASNDNH | 10.05 | 11.81 | 1.07 | 2.61 |
| ATLVT012XX2150 | 47084 | SSQTGGATNDNH | 9.52 | 10.46 | 2.18 | 4.82 | ATLVT012XX2196 | 47130 | SSTAGGASNDNT | 6.25 | 9.42 | 0.65 | 4.03 |
| ATLVT012XX2151 | 47085 | SSQTGGATNDNT | 5.49 | 5.41 | 0.37 | 1.4 | ATLVT012XX2197 | 47131 | SSTAGGATNDNA | 7.08 | 9.46 | 1.03 | 3.88 |
| ATLVT012XX2152 | 47086 | SSQTGGSSNDNA | 2.92 | 1.29 | 0.82 | 2.37 | ATLVT012XX2198 | 47132 | SSTAGGATNDNH | 10.47 | 12.47 | 0.98 | 3.49 |
| ATLVT012XX2153 | 47087 | SSQTGGSSNDNH | 3.15 | 0.98 | −0.14 | 2.17 | ATLVT012XX2199 | 47133 | SSTAGGATNDNT | 6.22 | 8.64 | 1.56 | 3.1 |
| ATLVT012XX2154 | 47088 | SSQTGGSSNDNT | 3.25 | 1.63 | 0.09 | 1.65 | ATLVT012XX2200 | 47134 | SSTAGGSSNDNA | 5.42 | 5.4 | 0.59 | 3.84 |
| ATLVT01XXX2155 | 47089 | SSQTGGSTNDNA | 3.87 | 5.78 | 1.17 | 2.84 | ATLVT012XX2201 | 47135 | SSTAGGSSNDNH | 4.48 | 3.35 | 1.6 | 4.26 |
| ATLVT012XX2156 | 47090 | SSQTGGSTNDNH | 6.36 | 6.28 | 1.12 | 4.16 | ATLVT012XX2202 | 47136 | SSTAGGSSNDNT | 4.91 | 5.66 | 1.1 | 3.59 |
| ATLVT012XX2157 | 47091 | SSQTGGSTNDNT | 3.8 | 2.69 | 0.36 | 1.49 | ATLVT012XX2203 | 47137 | SSTAGGSTNDNA | 7.17 | 7.86 | −0.09 | 4.47 |
| ATLVT012XX2158 | 47092 | SSTAAAASNDNA | 0.47 | −0.95 | 0.27 | 1.95 | ATLVT012XX2204 | 47138 | SSTAGGSTNDNH | 7.79 | 8.59 | 1.17 | 5.43 |
| ATLVT012XX2159 | 47093 | SSTAAAASNDNE | 0.11 | 0.63 | −0.7 | 0.92 | ATLVT012XX2205 | 47139 | SSTAGGSTNDNT | 6.66 | 6.91 | 0.14 | 4.2 |
| ATLVT012XX2160 | 47094 | SSTAAAASNDNT | 1.85 | −1.31 | −0.44 | 1.18 | ATLVT012XX2206 | 47140 | SSTSAAASNDNA | 0.07 | −0.92 | 1.15 | 0.18 |
| ATLVT012XX2161 | 47095 | SSTAAAATNDNA | −1.03 | −1.24 | −0.34 | 0.1 | ATLVT012XX2207 | 47141 | SSTSAAASNDNH | 1.15 | −0.77 | −0.17 | −0.81 |
| ATLVT012XX2162 | 47096 | SSTAAAATNDNH | −0.57 | 0.71 | 0 | 0.33 | ATLVT012XX2208 | 47142 | SSTSAAASNDNT | 3.32 | −0.98 | −1.08 | −0.15 |
| ATLVT012XX2209 | 47143 | SSTSAAATNDNA | 1.62 | −0.62 | 0.72 | −0.3 | ATLVT012XX2255 | 47189 | SSTTAAASNDNH | 1.46 | 2.19 | 0.2 | 2.35 |
| ATLVT012XX2210 | 47144 | SSTSAAATNDNH | 1.71 | −0.17 | 0.36 | 1.31 | ATLVT012XX2256 | 47190 | SSTTAAASNDNT | 2.4 | 0.59 | −0.29 | −0.05 |
| ATLVT012XX2211 | 47145 | SSTSAAATNDNT | 0.38 | 0.52 | 0.7 | 0.4 | ATLVT012XX2257 | 47191 | SSTTAAATNDNA | 0.32 | 1.72 | 0.92 | 1.97 |
| ATLVT012XX2212 | 47146 | SSTSAASSNDNA | 1.45 | −0.04 | 0.07 | 0 | ATLVT012XX2258 | 47192 | SSTTAAATNDNH | 0.37 | 0.54 | −0.46 | 1.31 |
| ATLVT012XX2213 | 47147 | SSTSAASSNDNH | 0.05 | −0.88 | 0.22 | 0.02 | ATLVT012XX2259 | 47193 | SSTTAAATNDNT | 0.72 | −0.22 | 0.16 | 0.18 |
| ATLVT012XX2214 | 47148 | SSTSAASSNDNT | 2.02 | 0.47 | 0.99 | 1.94 | ATLVT012XX2260 | 47194 | SSTTAASSNDNA | −0.29 | −1.82 | 0.69 | −1.26 |
| ATLVT012XX2215 | 47149 | SSTSAASTNDNA | 0.12 | −0.3 | −0.51 | −0.82 | ATLVT012XX2261 | 47195 | SSTTAASSNDNH | −0.89 | 0.7 | −0.02 | 0.32 |
| ATLVT012XX2216 | 47150 | SSTSAASTNDNH | −0.38 | 0.92 | −0.2 | 0.12 | ATLVT012XX2262 | 47196 | SSTTAASSNDNT | −0.08 | 0.04 | 0.41 | 0.6 |
| ATLVT012XX2217 | 47151 | SSTSAASTNDNT | 0.3 | −0.09 | 0.07 | −0.66 | ATLVT012XX2263 | 47197 | SSTTAASTNDNA | −0.17 | −0.01 | 0.25 | −0.76 |
| ATLVT012XX2218 | 47152 | SSTSAGASNDNA | 2.36 | 1.53 | 1.15 | 0.7 | ATLVT012XX2264 | 47198 | SSTTAASTNDNH | −1.2 | −0.43 | 0.19 | 1.85 |
| ATLVT012XX2219 | 47153 | SSTSAGASNDNH | 0.22 | 1.66 | −0.97 | 1.57 | ATLVT012XX2265 | 47199 | SSTTAASTNDNT | 1.69 | 1.59 | −0.57 | −0.64 |
| ATLVT012XX2220 | 47154 | SSTSAGASNDNT | 0.16 | 1.02 | 0.93 | 1.31 | ATLVT012XX2266 | 47200 | SSTTAGASNDNA | 0.71 | 0.8 | −0.42 | 2.52 |
| ATLVT012XX2221 | 47155 | SSTSAGATNDNA | −0.18 | 3.36 | 0.76 | −0.06 | ATLVT012XX2267 | 47201 | SSTTAGASNDNH | 0.64 | 2.38 | −0.1 | 2.42 |
| ATLVT012XX2222 | 47156 | SSTSAGATNDNH | 3.08 | 5.55 | 1.09 | 3.77 | ATLVT012XX2268 | 47202 | SSTTAGASNDNT | 1.23 | 1.22 | 0.08 | 1.12 |
| ATLVT012XX2223 | 47157 | SSTSAGATNDNT | 1.34 | 1.78 | −0.13 | 0.79 | ATLVT012XX2269 | 47203 | SSTTAGATNDNA | −0.33 | 2.55 | −0.68 | −0.24 |
| ATLVT012XX2224 | 47158 | SSTSAGSSNDNA | −0.24 | 1.21 | 0.73 | 0.95 | ATLVT012XX2270 | 47204 | SSTTAGATNDNH | 3.25 | 2.23 | 0.42 | 3.42 |
| ATLVT012XX2225 | 47159 | SSTSAGSSNDNH | −1.12 | −0.57 | 0.33 | 1.37 | ATLVT012XX2271 | 47205 | SSTTAGATNDNT | 0.03 | 0.08 | −0.39 | 0.49 |
| ATLVT012XX2226 | 47160 | SSTSAGSSNDNT | 0.86 | 2.31 | 0.78 | 0.39 | ATLVT012XX2272 | 47206 | SSTTAGSSNDNA | 0.72 | 0.42 | 1.01 | −0.13 |
| ATLVT012XX2227 | 47161 | SSTSAGSTNDNA | −0.1 | 0.27 | 0.67 | 0.22 | ATLVT012XX2273 | 47207 | SSTTAGSSNDNH | −0.05 | −0.21 | −0.51 | −1.45 |
| ATLVT012XX2228 | 47162 | SSTSAGSTNDNH | 0.57 | 2.56 | 0.66 | 1.69 | ATLVT012XX2274 | 47208 | SSTTAGSSNDNT | 2.06 | 2.73 | −0.07 | −1.11 |
| ATLVT012XX2229 | 47163 | SSTSAGSTNDNT | 1.76 | 2.33 | 0.8 | 1.49 | ATLVT012XX2275 | 47209 | SSTTAGSTNDNA | 0.32 | −1.5 | −0.25 | 1.44 |
| ATLVT012XX2230 | 47164 | SSTSGAASNDNA | 0.74 | −0.26 | 0.49 | 0.4 | ATLVT012XX2276 | 47210 | SSTTAGSTNDNH | −0.38 | −1.4 | −0.28 | 1.51 |
| ATLVT012XX2231 | 47165 | SSTSGAASNDNH | 1.78 | 0.9 | 0.37 | −0.48 | ATLVT012XX2277 | 47211 | SSTTAGSTNDNT | 0.6 | −0.13 | 0.19 | 0.53 |
| ATLVT012XX2232 | 47166 | SSTSGAASNDNT | 0.33 | −0.44 | −0.72 | −0.51 | ATLVT012XX2278 | 47212 | SSTTGAASNDNA | 1.42 | −0.08 | −0.9 | 0.95 |
| ATLVT012XX2233 | 47167 | SSTSGAATNDNA | 1.34 | 2.29 | 1.43 | 3.29 | ATLVT012XX2279 | 47213 | SSTTGAASNDNH | 1.39 | 0.75 | 0.17 | 1.07 |
| ATLVT012XX2234 | 47168 | SSTSGAATNDNH | 1.93 | 3.46 | 0.53 | 3.82 | ATLVT012XX2280 | 47214 | SSTTGAASNDNT | 0.84 | 4.59 | −0.07 | −0.12 |
| ATLVT012XX2235 | 47169 | SSTSGAATNDNT | 1.08 | 1.99 | −0.72 | 0.39 | ATLVT012XX2281 | 47215 | SSTTGAATNDNA | 2.54 | 0.95 | −1.26 | −0.69 |
| ATLVT012XX2236 | 47170 | SSTSGASSNDNA | 1.51 | 0.52 | 0.39 | 0.02 | ATLVT012XX2282 | 47216 | SSTTGAATNDNH | 1.79 | 1.44 | 0.65 | 2.28 |
| ATLVT012XX2237 | 47171 | SSTSGASSNDNH | 0.47 | 0.55 | 0.51 | 0.63 | ATLVT012XX2283 | 47217 | SSTTGAATNDNT | 3.23 | 0.99 | −0.58 | −2.06 |
| ATLVT012XX2238 | 47172 | SSTSGASSNDNT | 0.4 | 1.02 | 0.16 | −0.56 | ATLVT012XX2284 | 47218 | SSTTGASSNDNA | 1.76 | 2.71 | 0.82 | −0.97 |
| ATLVT012XX2239 | 47173 | SSTSGASTNDNA | 0.43 | −0.44 | 1.06 | 0.28 | ATLVT012XX2285 | 47219 | SSTTGASSNDNH | 1.3 | 0.56 | 0.71 | −0.16 |
| ATLVT012XX2240 | 47174 | SSTSGASTNDNH | 1.47 | 1.23 | 0.45 | 2.48 | ATLVT012XX2286 | 47220 | SSTTGASSNDNT | 2.6 | 1.2 | 0.26 | −0.26 |
| ATLVT012XX2241 | 47175 | SSTSGASTNDNT | 0.71 | 0.79 | 0.9 | 0.26 | ATLVT012XX2287 | 47221 | SSTTGASTNDNA | 0.57 | −0.46 | −0.24 | −0.14 |
| ATLVT012XX2242 | 47176 | SSTSGGASNDNA | 5.97 | 7.63 | 1.49 | 3.04 | ATLVT012XX2288 | 47222 | SSTTGASTNDNH | −0.19 | 1.96 | 0.19 | 0.71 |
| ATLVT012XX2243 | 47177 | SSTSGGASNDNH | 8.73 | 9.31 | 2.04 | 4.85 | ATLVT012XX2289 | 47223 | SSTTGASTNDNT | 1.98 | 0.58 | 0.72 | 0.37 |
| ATLVT012XX2244 | 47178 | SSTSGGASNDNT | 6.29 | 6.02 | 1.48 | 3.48 | ATLVT012XX2290 | 47224 | SSTTGGASNDNA | 3.64 | 5.2 | 1.54 | 3.31 |
| ATLVT012XX2245 | 47179 | SSTSGGATNDNA | 6.57 | 8.2 | 0.91 | 4.28 | ATLVT012XX2291 | 47225 | SSTTGGASNDNH | 7.54 | 9.81 | 1.57 | S.il |
| ATLVT012XX2246 | 47180 | SSTSGGATNDNH | 10.29 | 12.33 | 1.17 | 4.14 | ATTVT012XX2292 | 47226 | SSTTGGASNDNT | 5.14 | 4.91 | 0.1 | 1.94 |
| ATLVT012XX2247 | 47181 | SSTSGGATNDNT | 7.12 | 7.09 | 0.12 | 3.62 | ATLVT012XX2293 | 47227 | SSTTGGATNDNA | 5.26 | 6.72 | 1.25 | 3.83 |
| ATLVT012XX2248 | 47182 | SSTSGGSSNDNA | 5.06 | 5.31 | 0.24 | 3.53 | ATLVT012XX2294 | 47228 | SSTTGGATNDNH | 10.02 | 12.11 | 1.06 | 4.6 |
| ATLVT012XX2249 | 47183 | SSTSGGSSNDNH | 4.03 | 5.7 | 0.8 | 2.3 | ATLVT012XX2295 | 47229 | SSTTGGATNDNT | 5.42 | 5.9 | 0.48 | 2.44 |
| ATLVT012XX2250 | 47184 | SSTSGGSSNDNT | 5.15 | 4.36 | 0.24 | 2.79 | ATLVT012XX2296 | 47230 | SSTTGGSSNDNA | 3.86 | 6.03 | 0.18 | 2.12 |
| ATLVT012XX2251 | 47185 | SSTSGGSTNDNA | 6.05 | 6.07 | 2.12 | 3.89 | ATLVT012XX2297 | 47231 | SSTTGGSSNDNH | 2.81 | 4.13 | 0.49 | 1.76 |
| ATLVT012XX2252 | 47186 | SSTSGGSTNDNH | 7.1 | 6.39 | 1.1 | 3.65 | ATLVT012XX2298 | 47232 | SSTTGGSSNDNT | 3.87 | 3.52 | −0.53 | 0.68 |
| ATLVT012XX2253 | 47187 | SSTSGGSTNDNT | 5.3 | 5.23 | 0.97 | 3.15 | ATLVT012XX2299 | 47233 | SSTTGGSTNDNA | 4.24 | 6.84 | 0.68 | 2.22 |
| ATLVT012XX2254 | 47188 | SSTTAAASNDNA | 0.39 | 2.59 | 0.51 | −0.58 | ATLVT012XX2300 | 47234 | SSTTGGSTNDNH | 5.59 | 5.32 | 0.3 | 3.25 |
| ATLVT012XX2301 | 47235 | SSTTGGSTNDNT | 5.45 | 5.4 | 1.06 | 1.88 | ATLVT012XX2347 | 47281 | TGAAGGSTNDNA | −0.46 | −0.11 | −2.77 | −2.63 |
| ATLVT012XX2302 | 47236 | TGAAAAASNDNA | −2.83 | 2 | −3.42 | 0.28 | ATLVT012XX2348 | 47282 | TGAAGGSTNDNH | 3.8 | 6.3 | 0.14 | 1.9 |
| ATLVT012XX2303 | 47237 | TGAAAAASNDNH | −0.66 | 1.51 | −2.6 | −1.72 | ATLVT012XX2349 | 47283 | TGAAGGSTNDNT | 0.51 | 1.3 | −3.37 | −1.88 |
| ATLVT012XX2304 | 47238 | TGAAAAASNDNT | −2.16 | 0.11 | −3.63 | −0.37 | ATLVT012XX2350 | 47284 | TGASAAASNDNA | 0.44 | −1.47 | −0.5 | 1.32 |
| ATLVT012XX2305 | 47239 | TGAAAAATNDNA | −1.96 | 0.4 | 0.68 | −2.15 | ATLVT012XX2351 | 47285 | TGASAAASNDNH | 0.24 | −0.72 | −1.41 | −1.24 |
| ATLVT012XX2306 | 47240 | TGAAAAATNDNH | −1.14 | 3.15 | −1.59 | −1.39 | ATLVT012XX2352 | 47286 | TGASAAASNDNT | 1.88 | 0.25 | −0.86 | 1.81 |
| ATLVT012XX2307 | 47241 | TGAAAAATNDNT | −1.77 | −0.74 | 0.25 | −2.76 | ATLVT012XX2353 | 47287 | TGASAAATNDNA | 0.44 | −0.73 | −0.69 | 1.16 |
| ATLVT012XX2308 | 47242 | TGAAAASSNDNA | −2.82 | 1.72 | −2.45 | −2.08 | ATLVT012XX2354 | 47288 | TGASAAATNDNH | 0.19 | −0.72 | −1.9 | 0.69 |
| ATLVT012XX2309 | 47243 | TGAAAASSNDNH | −2.32 | −0.07 | −1.06 | −0.1 | ATLVT012XX2355 | 47289 | TGASAAATNDNT | −0.66 | 0.53 | −0.89 | 0.18 |
| ATLVT012XX2310 | 47244 | TGAAAASSNDNT | −1.77 | 1.02 | −1.25 | −0.84 | ATLVT012XX2356 | 47290 | TGASAASSNDNA | 3.05 | −0.26 | −1.48 | −1.15 |
| ATLVT012XX2311 | 47245 | TGAAAASTNDNA | −2.79 | 0.61 | −0.01 | −1.95 | ATLVT012XX2357 | 47291 | TGASAASSNDNH | −0.31 | −1.26 | −0.92 | 0.63 |
| ATLVT012XX2312 | 47246 | TGAAAASTNDNH | −3.14 | −0.96 | −1.43 | −1.7 | ATLVT012XX2358 | 47292 | TGASAASSNDNT | 0.43 | −1.61 | −1.2 | 1.48 |
| ATLVT012XX2313 | 47247 | TGAAAASTNDNT | −3.04 | −1.32 | −0.96 | −2.86 | ATLVT012XX2359 | 47293 | TGASAASTNDNA | −0.23 | −0.43 | 0.14 | 0.6 |
| ATLVT012XX2314 | 47248 | TGAAAGASNDNA | −0.66 | −1.74 | 0 | −2.75 | ATLVT012XX2360 | 47294 | TGASAASTNDNH | −1.42 | 1.75 | −1.27 | −0.95 |
| ATLVT012XX2315 | 47249 | TGAAAGASNDNH | 0.59 | −0.9 | −0.34 | −1.6 | ATLVT012XX2361 | 47295 | TGASAASTNDNT | −0.15 | −1.19 | −0.88 | −0.8 |
| ATLVT012XX2316 | 47250 | TGAAAGASNDNT | 1.35 | −1.1 | −2.02 | 0.17 | ATLVT012XX2362 | 47296 | TGASAGASNDNA | 0.08 | −0.07 | −0.78 | 0.16 |
| ATLVT012XX2317 | 47251 | TGAAAGATNDNA | 0.61 | −0.33 | −2.95 | −2.89 | ATLVT012XX2363 | 47297 | TGASAGASNDNH | −0.16 | 1.81 | −0.73 | −0.27 |
| ATLVT012XX2318 | 47252 | TGAAAGATNDNH | 0.09 | 0.91 | −2.24 | −2.11 | ATLVT012XX2364 | 47298 | TGASAGASNDNT | −1.17 | −0.73 | −0.73 | −0.78 |
| ATLVT012XX2319 | 47253 | TGAAAGATNDNT | −0.42 | −1.8 | −4.15 | −2.19 | ATLVT012XX2365 | 47299 | TGASAGATNDNA | −1.75 | −0.59 | −0.38 | 0.4 |
| ATLVT012XX2320 | 47254 | TGAAAGSSNDNA | −1.5 | −3.29 | −2.2 | −2.58 | ATLVT012XX2366 | 47300 | TGASAGATNDNH | −0.79 | 3.18 | −0.96 | 1.25 |
| ATLVT012XX2321 | 47255 | TGAAAGSSNDNH | −2.81 | −2.6 | −4.49 | −1.79 | ATLVT012XX2367 | 47301 | TGASAGATNDNT | −1.08 | 0.97 | −0.38 | −0.41 |
| ATLVT012XX2322 | 47256 | TGAAAGSSNDNT | −1.58 | −2.01 | −3.69 | −2.23 | ATLVT012XX2368 | 47302 | TGASAGSSNDNA | −0.26 | −0.45 | −0.45 | −1.61 |
| ATLVT012XX2323 | 47257 | TGAAAGSTNDNA | −2.43 | −2.76 | −3.64 | −2.29 | ATLVT012XX2369 | 47303 | TGASAGSSNDNH | 0.69 | 2.23 | 0.31 | −1.28 |
| ATLVT012XX2324 | 47258 | TGAAAGSTNDNH | −2.03 | −1.98 | −2.49 | −1.94 | ATLVT012XX2370 | 47304 | TGASAGSSNDNT | 1.09 | 0.41 | −1.14 | −1.41 |
| ATLVT012XX2325 | 47259 | TGAAAGSTNDNT | −1.84 | −1.67 | −3.14 | −2.85 | ATLVT012XX2371 | 47305 | TGASAGSTNDNA | −2.35 | 3.41 | 0.11 | −1.15 |
| ATLVT012XX2326 | 47260 | TGAAGAASNDNA | −1.43 | 0.07 | −3.26 | −1.66 | ATLVT012XX2372 | 47306 | TGASAGSTNDNH | −1.43 | 1.84 | 1.15 | 0.45 |
| ATLVT012XX2327 | 47261 | TGAAGAASNDNH | −1.32 | −0.33 | −3.98 | −2.36 | ATLVT012XX2373 | 47307 | TGASAGSTNDNT | −1.43 | 0.64 | −0.43 | −1.78 |
| ATLVT012XX2328 | 47262 | TGAAGAASNDNT | −0.74 | −0.96 | −2.81 | −1.61 | ATLVT012XX2374 | 47308 | TGASGAASNDNA | −0.47 | 0.16 | −1.04 | 1.66 |
| ATLVT012XX2329 | 47263 | TGAAGAATNDNA | 1.09 | −0.62 | −2.97 | −0.92 | ATLVT012XX2375 | 47309 | TGASGAASNDNH | −1.11 | −1.48 | 0.34 | 1.15 |
| ATLVT012XX2330 | 47264 | TGAAGAATNDNH | −0.28 | −0.68 | −4.31 | −1.67 | ATLVT012XX2376 | 47310 | TGASGAASNDNT | −2.55 | −2.29 | −3.52 | 1.76 |
| ATLVT012XX2331 | 47265 | TGAAGAATNDNT | −2.95 | 0.41 | −5 | −2.83 | ATLVT012XX2377 | 47311 | TGASGAATNDNA | −0.63 | −1.33 | −2.48 | −2.15 |
| ATLVT012XX2332 | 47266 | TGAAGASSNDNA | −2.73 | −1.39 | −2.45 | −3.91 | ATLVT012XX2378 | 47312 | TGASGAATNDNH | 0.83 | −0.92 | −1.9 | −1.68 |
| ATLVT012XX2333 | 47267 | TGAAGASSNDNH | −2.58 | −1.2 | −3.23 | −4.23 | ATLVT012XX2379 | 47313 | TGASGAATNDNT | −0.12 | −0.36 | −2.17 | −1.5 |
| ATLVT012XX2334 | 47268 | TGAAGASSNDNT | −2.1 | 2.01 | −3.45 | −2.02 | ATLVT012XX2380 | 47314 | TGASGASSNDNA | −1.28 | −0.52 | −1.53 | −0.5 |
| ATLVT012XX2335 | 47269 | TGAAGASTNDNA | 0.55 | 0.49 | 0.28 | −0.07 | ATLVT012XX2381 | 47315 | TGASGASSNDNH | −1.99 | −1.11 | −1.54 | −1.53 |
| ATLVT012XX2336 | 47270 | TGAAGASTNDNH | −1.56 | −0.5 | −1.64 | −1.35 | ATLVT012XX2382 | 47316 | TGASGASSNDNT | −0.85 | 1.54 | −0.03 | −2.12 |
| ATLVT012XX2337 | 47271 | TGAAGASTNDNT | 0.33 | −1.48 | −0.39 | −1.91 | ATLVT012XX2383 | 47317 | TGASGASTNDNA | −1.88 | −0.23 | −0.1 | 0.48 |
| ATLVT012XX2338 | 47272 | TGAAGGASNDNA | 4.25 | 4.95 | −0.78 | 3.18 | ATLVT012XX2384 | 47318 | TGASGASTNDNH | 0.09 | 0.53 | −0.62 | 1.15 |
| ATLVT012XX2339 | 47273 | TGAAGGASNDNH | 4.78 | 4.9 | −0.81 | 1.14 | ATLVT012XX2385 | 47319 | TGASGASTNDNT | 0.52 | −1.19 | −0.56 | −0.12 |
| ATLVT012XX2340 | 47274 | TGAAGGASNDNT | 6.31 | 7.16 | 1.25 | 2.98 | ATLVT012XX2386 | 47320 | TGASGGASNDNA | −0.39 | 0.86 | −0.99 | −0.2 |
| ATLVT012XX2341 | 47275 | TGAAGGATNDNA | 2.4 | 4.15 | −1.77 | 0.46 | ATLVT012XX2387 | 47321 | TGASGGASNDNH | 1.89 | 0.53 | −0.51 | 1.95 |
| ATLVT012XX2342 | 47276 | TGAAGGATNDNH | 2.39 | 4.55 | −3.84 | 0.97 | ATLVT012XX2388 | 47322 | TGASGGASNDNT | 0.99 | 0.59 | −1.2 | 1.32 |
| ATLVT012XX2343 | 47277 | TGAAGGATNDNT | 0.74 | 1.25 | −2.54 | −1.99 | ATLVT012XX2389 | 47323 | TGASGGATNDNA | 2.05 | −0.36 | −2.55 | 0.25 |
| ATLVT012XX2344 | 47278 | TGAAGGSSNDNA | 0.16 | 1.32 | −2.43 | −2.05 | ATLVT012XX2390 | 47324 | TGASGGATNDNH | 3.64 | 5 | 0.18 | 4.28 |
| ATLVT012XX2345 | 47279 | TGAAGGSSNDNH | −2.15 | −0.03 | −3.32 | −3.13 | ATLVT012XX2391 | 47325 | TGASGGATNDNT | 1.49 | 0.88 | −1.4 | 0.07 |
| ATLVT012XX2346 | 47280 | TGAAGGSSNDNT | −0.24 | 0.04 | −2.68 | −3.94 | ATLVT012XX2392 | 47326 | TGASGGSSNDNA | 0.96 | 1.73 | 1.53 | −0.53 |
| ATLVT012XX2393 | 47327 | TGASGGSSNDNH | −0.17 | −0.25 | −1.16 | 1.27 | ATLVT012XX2439 | 47373 | TGATGGATNDNT | 4.18 | 4.23 | −1.4 | 1.75 |
| ATLVT012XX2394 | 47328 | TGASGGSSNDNT | 0.94 | 0.42 | 0.57 | −1.31 | ATLVT012XX2440 | 47374 | TGATGGSSNDNA | −0.54 | 0.59 | 0.27 | −0.35 |
| ATLVT012XX2395 | 47329 | TGASGGSTNDNA | 2.11 | −0.19 | −0.93 | −0.24 | ATLVT012XX2441 | 47375 | TGATGGSSNDNH | −1.25 | 3.98 | 0.74 | −0.68 |
| ATLVT012XX2396 | 47330 | TGASGGSTNDNH | 2.48 | −0.61 | 0.39 | 0.45 | ATLVT012XX2442 | 47376 | TGATGGSSNDNT | 1.82 | 3.29 | −0.8 | 0.24 |
| ATLVT012XX2397 | 47331 | TGASGGSTNDNT | 1.28 | −0.17 | −0.48 | −0.49 | ATLVT012XX2443 | 47377 | TGATGGSTNDNA | 1.68 | 1.28 | −0.29 | −0.03 |
| ATLVT012XX2398 | 47332 | TGATAAASNDNA | −0.26 | −0.77 | 0.82 | 0.79 | ATLVT012XX2444 | 47378 | TGATGGSTNDNH | 2.74 | 1.92 | 0.21 | 2.52 |
| ATLVT012XX2399 | 47333 | TGATAAASNDNH | −0.31 | −0.8 | −1.38 | −0.26 | ATLVT012XX2445 | 47379 | TGATGGSTNDNT | 2.64 | 2.84 | 0.38 | 1.03 |
| ATLVT012XX2400 | 47334 | TGATAAASNDNT | −0.89 | | −1.4 | −0.48 | ATLVT012XX2446 | 47380 | TGEAAAASNDNA | −0.36 | −1.37 | −1.07 | −0.51 |
| ATLVT012XX2401 | 47335 | TGATAAATNDNA | −2.06 | 2.24 | 0.91 | −0.41 | ATLVT012XX2447 | 47381 | TGEAAAASNDNH | 0.14 | −0.14 | −1 | −2.06 |
| ATLVT012XX2402 | 47336 | TGATAAATNDNH | −0.88 | 0.45 | 0.36 | 1.51 | ATLVT012XX2448 | 47382 | TGEAAAASNDNT | −1.72 | 0.06 | −2.46 | −2.93 |
| ATLVT012XX2403 | 47337 | TGATAAATNDNT | −1.74 | −0.99 | −0.45 | 0.27 | ATLVT012XX2449 | 47383 | TGEAAAATNDNA | 2.7 | 1.89 | −1.03 | −0.42 |
| ATLVT012XX2404 | 47338 | TGATAASSNDNA | 0.35 | −1.62 | 0.01 | 0.79 | ATLVT012XX2450 | 47384 | TGEAAAATNDNH | 1.37 | −0.42 | −0.74 | −0.07 |
| ATLVT012XX2405 | 47339 | TGATAASSNDNH | −0.54 | 1.06 | −0.37 | −0.13 | ATLVT012XX2451 | 47385 | TGEAAAATNDNT | 1.19 | −0.94 | −1 | −0.16 |
| ATLVT012XX2406 | 47340 | TGATAASSNDNT | 0.33 | −0.76 | −0.72 | −0.41 | ATLVT012XX2452 | 47386 | TGEAAASSNDNA | 1.79 | −0.52 | −1.26 | −0.2 |
| ATLVT012XX2407 | 47341 | TGATAASTNDNA | 0.28 | −0.09 | −0.49 | 0.6 | ATLVT012XX2453 | 47387 | TGEAAASSNDNH | 0.18 | 0.02 | −1.66 | −1.79 |
| ATLVT012XX2408 | 47342 | TGATAASTNDNH | 1.62 | −0.17 | −0.51 | 0.25 | ATLVT012XX2454 | 47388 | TGEAAASSNDNT | 1.68 | −0.01 | −0.49 | −0.85 |
| ATLVT012XX2409 | 47343 | TGATAASTNDNT | −0.31 | 2.09 | −0.75 | 0.39 | ATLVT012XX2455 | 47389 | TGEAAASTNDNA | −0.84 | 1.3 | −1.45 | 0.17 |
| ATLVT012XX2410 | 47344 | TGATAGASNDNA | 2.14 | 0.5 | −0.12 | −1.03 | ATLVT012XX2456 | 47390 | TGEAAASTNDNH | −1.8 | 0.62 | −0.7 | 1.25 |
| ATLVT012XX2411 | 47345 | TGATAGASNDNH | 1.91 | 4.73 | 0.45 | −1.31 | ATLVT012XX2457 | 47391 | TGEAAASTNDNT | −0.43 | −1.38 | −0.38 | 0.83 |
| ATLVT012XX2412 | 47346 | TGATAGASNDNT | 2.07 | 1.64 | −1.17 | −0.87 | ATLVT012XX2458 | 47392 | TGEAAGASNDNA | −2.06 | 0.63 | −1.46 | 1.7 |
| ATLVT012XX2413 | 47347 | TGATAGATNDNA | −1.1 | 1.48 | −0.5 | 0.41 | ATLVT012XX2459 | 47393 | TGEAAGASNDNH | −2.7 | −1.4 | 0.23 | −0.15 |
| ATLVT012XX2414 | 47348 | TGATAGATNDNH | 1.14 | 5.85 | 0.93 | 0.33 | ATLVT012XX2460 | 47394 | TGEAAGASNDNT | −2.5 | −0.97 | 0.29 | −2.13 |
| ATLVT012XX2415 | 47349 | TGATAGATNDNT | 0.87 | 1.4 | −1.74 | −1.04 | ATLVT012XX2461 | 47395 | TGEAAGATNDNA | −2.64 | −0.92 | −1.04 | −1.83 |
| ATLVT012XX2416 | 47350 | TGATAGSSNDNA | 0.07 | −0.9 | 0.16 | −1.2 | ATLVT012XX2462 | 47396 | TGEAAGATNDNH | −2.49 | −0.74 | −1.97 | −0.2 |
| ATLVT012XX2417 | 47351 | TGATAGSSNDNH | 0.48 | 1.05 | −0.32 | −1.3 | ATLVT012XX2463 | 47397 | TGEAAGATNDNT | −2.23 | −1.38 | 1.45 | −0.68 |
| ATLVT012XX2418 | 47352 | TGATAGSSNDNT | −0.57 | −1.13 | −0.08 | 1.96 | ATLVT012XX2464 | 47398 | TGEAAGSSNDNA | 0.43 | −1.57 | −1.06 | −0.77 |
| ATLVT012XX2419 | 47353 | TGATAGSTNDNA | −1.05 | 2.28 | 0.1 | −0.1 | ATLVT012XX2465 | 47399 | TGEAAGSSNDNH | −2.03 | −1.01 | −1.17 | −2.24 |
| ATLVT012XX2420 | 47354 | TGATAGSTNDNH | −0.63 | 1 | −1.49 | −0.22 | ATLVT012XX2466 | 47400 | TGEAAGSSNDNT | −0.15 | −2.43 | 0.28 | −2.97 |
| ATLVT012XX2421 | 47355 | TGATAGSTNDNT | −0.34 | 0.53 | −1.2 | −0.91 | ATLVT012XX2467 | 47401 | TGEAAGSTNDNA | 0.22 | −1.98 | 0.14 | −1.12 |
| ATLVT012XX2422 | 47356 | TGATGAASNDNA | 0.39 | −0.35 | −0.7 | 0.15 | ATLVT012XX2468 | 47402 | TGEAAGSTNDNH | −0.15 | −1.31 | −0.39 | −0.94 |
| ATLVT012XX2423 | 47357 | TGATGAASNDNH | −1.54 | −1.57 | −1.98 | −0.3 | ATLVT012XX2469 | 47403 | TGEAAGSTNDNT | 0.03 | −1.29 | −1.6 | −2.31 |
| ATLVT012XX2424 | 47358 | TGATGAASNDNT | −1.95 | −2.06 | −3.08 | 0.22 | ATLVT012XX2470 | 47404 | TGEAGAASNDNA | −0.49 | 2.8 | −2.35 | −3.1 |
| ATLVT012XX2425 | 47359 | TGATGAATNDNA | −0.87 | 0.5 | −0.56 | −1.31 | ATLVT012XX2471 | 47405 | TGEAGAASNDNH | −2.07 | 0.28 | −2.43 | 0.64 |
| ATLVT012XX2426 | 47360 | TGATGAATNDNH | 3.5 | 1.68 | −0.64 | 1.7 | ATLVT012XX2472 | 47406 | TGEAGAASNDNT | −2.45 | −1.17 | −2.41 | −1.88 |
| ATLVT012XX2427 | 47361 | TGATGAATNDNT | 1.45 | −0.78 | −1.06 | −0.45 | ATLVT012XX2473 | 47407 | TGEAGAATNDNA | −2.97 | 0.99 | −1.09 | −2.93 |
| ATLVT012XX2428 | 47362 | TGATGASSNDNA | 1.58 | 2.43 | −0.46 | −2.23 | ATLVT012XX2474 | 47408 | TGEAGAATNDNH | −3.56 | 1.55 | −1.32 | −1.25 |
| ATLVT012XX2429 | 47363 | TGATGASSNDNH | −1.27 | 3.11 | 1.25 | −2.37 | ATLVT012XX2475 | 47409 | TGEAGAATNDNT | −2.63 | 4.38 | −1.87 | −1.69 |
| ATLVT012XX2430 | 47364 | TGATGASSNDNT | −0.35 | −0.27 | 0.16 | −1.24 | ATLVT012XX2476 | 47410 | TGEAGASSNDNA | 2.72 | −0.78 | −1.15 | −1.17 |
| ATLVT012XX2431 | 47365 | TGATGASTNDNA | 1 | 2.21 | −1.63 | −0.88 | ATLVT012XX2477 | 47411 | TGEAGASSNDNH | 0.84 | −0.15 | −0.14 | −3.17 |
| ATLVT012XX2432 | 47366 | TGATGASTNDNH | 1.92 | 2.24 | −0.15 | 0.22 | ATLVT012XX2478 | 47412 | TGEAGASSNDNT | 5.18 | −1.75 | −0.44 | −1.94 |
| ATLVT012XX2433 | 47367 | TGATGASTNDNT | 3.11 | 1.02 | −2.4 | −0.21 | ATLVT012XX2479 | 47413 | TGEAGASTNDNA | −0.7 | −0.47 | 0.67 | −3.1 |
| ATLVT012XX2434 | 47368 | TGATGGASNDNA | 0.03 | 0.64 | −1.21 | 0.75 | ATLVT012XX2480 | 47414 | TGEAGASTNDNH | −1.16 | −0.04 | 0.08 | −0.1 |
| ATLVT012XX2435 | 47369 | TGATGGASNDNH | 1.02 | 3.66 | −0.43 | 2.76 | ATLVT012XX2481 | 47415 | TGEAGASTNDNT | −1.77 | 1.53 | −0.94 | −0.7 |
| ATLVT012XX2436 | 47370 | TGATGGASNDNT | 1.54 | 1 | −0.03 | 1.51 | ATLVT012XX2482 | 47416 | TGEAGGASNDNA | 1.13 | 0.89 | −2.03 | −1.61 |
| ATLVT012XX2437 | 47371 | TGATGGATNDNA | 3.65 | 1.22 | 0.04 | −0.01 | ATLVT012XX2483 | 47417 | TGEAGGASNDNH | 1.52 | −0.35 | −2.62 | −0.65 |
| ATLVT012XX2438 | 47372 | TGATGGATNDNH | 6.36 | 4.93 | 0.04 | 2.21 | ATLVT012XX2484 | 47418 | TGEAGGASNDNT | 0.61 | 1.1 | −2.2 | −2.33 |
| ATLVT012XX2485 | 47419 | TGEAGGATNDNA | 0.29 | 1.6 | −0.94 | 0.61 | ATLVT012XX2531 | 47465 | TGESGGASNDNH | 1.91 | 3.93 | 0.07 | 0.41 |
| ATLVT012XX2486 | 47420 | TGEAGGATNDNH | 2.8 | 5.4 | 0.06 | 2.42 | ATLVT012XX2532 | 47466 | TGESGGASNDNT | 1.98 | 1.15 | −1.09 | −0.5 |
| ATLVT012XX2487 | 47421 | TGEAGGATNDNT | 1.43 | 3.88 | −1.66 | −0.68 | ATLVT012XX2533 | 47467 | TGESGGATNDNA | 1.42 | 3.35 | 0.33 | 0.17 |
| ATLVT012XX2488 | 47422 | TGEAGGSSNDNA | 3.19 | 0.65 | −0.52 | −0.47 | ATLVT012XX2534 | 47468 | TGESGGATNDNH | 6.49 | 2.84 | 0.39 | 2.12 |
| ATLVT012XX2489 | 47423 | TGEAGGSSNDNH | 0 | −0.44 | 0.13 | −1.74 | ATLVT012XX2535 | 47469 | TGESGGATNDNT | 2.7 | 3.13 | 0.68 | −0.55 |
| ATLVT012XX2490 | 47424 | TGEAGGSSNDNT | 0.89 | −0.07 | −0.6 | −1.75 | ATLVT012XX2536 | 47470 | TGESGGSSNDNA | 1.47 | 1.23 | −0.2 | −0.56 |
| ATLVT012XX2491 | 47425 | TGEAGGSTNDNA | 1.27 | 0.41 | −0.26 | 1.27 | ATLVT012XX2537 | 47471 | TGESGGSSNDNH | −0.18 | −0.97 | 0 | −0.1 |
| ATLVT012XX2492 | 47426 | TGEAGGSTNDNH | 0.76 | 1.01 | −0.51 | 0.49 | ATLVT012XX2538 | 47472 | TGESGGSSNDNT | 0.78 | 2.12 | −1.71 | −0.74 |
| ATLVT012XX2493 | 47427 | TGEAGGSTNDNT | 0.35 | −0.12 | −1.51 | −1.89 | ATLVT012XX2539 | 47473 | TGESGGSTNDNA | 1.79 | 2.44 | −0.27 | 0.02 |
| ATLVT012XX2494 | 47428 | TGESAAASNDNA | −0.2 | −0.86 | −0.83 | −1.61 | ATLVT012XX2540 | 47474 | TGESGGSTNDNH | 0.84 | −0.18 | 0.07 | 0.97 |
| ATLVT012XX2495 | 47429 | TGESAAASNDNH | −0.08 | −1.65 | −1.4 | 0.7 | ATLVT012XX2541 | 47475 | TGESGGSTNDNT | 1.82 | 0.39 | 0.31 | 0.86 |
| ATLVT012XX2496 | 47430 | TGESAAASNDNT | 0.36 | −1.17 | −1.51 | −1.72 | ATLVT012XX2542 | 47476 | TGETAAASNDNA | 2.13 | 0.69 | −0.13 | −1.75 |
| ATLVT012XX2497 | 47431 | TGESAAATNDNA | 1.97 | −0.71 | −0.52 | −0.08 | ATLVT012XX2543 | 47477 | TGETAAASNDNH | 1.86 | 2.44 | 0.13 | 0.37 |
| ATLVT012XX2498 | 47432 | TGESAAATNDNH | 2.63 | −0.71 | 0.23 | −0.16 | ATLVT012XX2544 | 47478 | TGETAAASNDNT | 1.04 | 0.31 | −1.06 | −2.12 |
| ATLVT012XX2499 | 47433 | TGESAAATNDNT | 1.8 | −1.68 | 0 | −1.93 | ATLVT012XX2545 | 47479 | TGETAAATNDNA | 1.02 | 0.49 | 0.6 | 0.48 |
| ATLVT012XX2500 | 47434 | TGESAASSNDNA | −0.42 | −2.43 | −0.92 | −2.43 | ATLVT012XX2546 | 47480 | TGETAAATNDNH | 1.62 | 1.27 | −1.02 | 0.92 |
| ATLVT012XX2501 | 47435 | TGESAASSNDNH | 2.28 | −2.17 | −1.23 | −0.77 | ATTVT012XX2547 | 47481 | TGETAAATNQNT | 3.33 | 2.45 | −0.98 | 0.3 |
| ATLVT012XX2502 | 47436 | TGESAASSNDNT | 0.52 | −2.19 | −1.26 | −1.78 | ATLVT012XX2548 | 47482 | TGETAASSNDNA | 0.17 | 0.12 | 0.68 | −3.11 |
| ATLVT012XX2503 | 47437 | TGESAASTNDNA | 0.65 | 0.94 | −0.22 | −1.81 | ATLVT012XX2549 | 47483 | TGETAASSNDNH | −1.37 | −0.91 | −0.29 | −2.58 |
| ATLVT012XX2504 | 47438 | TGESAASTNDNH | 2.75 | −0.65 | −0.69 | −2.36 | ATLVT012XX2550 | 47484 | TGETAASSNDNT | 1.19 | −1.4 | −0.43 | −2.45 |
| ATLVT012XX2505 | 47439 | TGESAASTNQNT | 2.31 | 1.44 | −0.68 | −0.67 | ATLVT012XX2551 | 47485 | TGETAASTNDNA | −2.53 | 1.01 | 0.88 | −0.28 |
| ATLVT012XX2506 | 47440 | TGESAGASNDNA | −1.28 | 1.86 | −0.18 | −2.27 | ATLVT012XX2552 | 47486 | TGETAASTNDNH | −2.93 | 2.52 | 0.56 | −1.34 |
| ATLVT012XX2507 | 47441 | TGESAGASNDNH | −0.57 | −0.62 | −1.56 | −0.31 | ATLVT012XX2553 | 47487 | TGETAASTNDNT | −1.01 | 2.56 | −0.49 | −1.98 |
| ATLVT012XX2508 | 47442 | TGESAGASNDNT | −0.28 | 0.69 | −2.53 | −2.59 | ATLVT012XX2554 | 47488 | TGETAGASNDNA | −1.3 | 1.35 | −0.23 | −0.42 |
| ATLVT012XX2509 | 47443 | TGESAGATNDNA | −2.26 | 0.17 | −2.11 | −1.75 | ATLVT012XX2555 | 47489 | TGETAGASNDNH | −1.85 | −0.77 | −1.07 | −1.63 |
| ATLVT012XX2510 | 47444 | TGESAGATNDNH | −0.85 | 0.7 | −0.92 | 0.49 | ATLVT012XX2556 | 47490 | TGETAGASNDNT | −1.5 | −0.78 | −0.24 | −0.24 |
| ATLVT012XX2511 | 47445 | TGESAGATNDNT | −1.62 | 1.9 | −2.02 | −1.45 | ATLVT012XX2557 | 47491 | TGETAGATNDNA | 0.08 | 2.47 | −0.02 | −1.89 |
| ATLVT012XX2512 | 47446 | TGESAGSSNDNA | −0.52 | −1.72 | −0.87 | −2.63 | ATLVT012XX2558 | 47492 | TGETAGATNDNH | −0.1 | −0.36 | 1.88 | 1.13 |
| ATLVT012XX2513 | 47447 | TGESAGSSNDNH | 1.01 | −1.95 | −1.29 | −0.95 | ATLVT012XX2559 | 47493 | TGETAGATNDNT | 1.02 | −0.56 | −0.87 | −0.73 |
| ATLVT012XX2514 | 47448 | TGESAGSSNDNT | 1.24 | −0.85 | −0.57 | −3.09 | ATLVT012XX2560 | 47494 | TGETAGSSNDNA | −0.28 | −1.51 | −0.16 | 0.2 |
| ATLVT012XX2515 | 47449 | TGESAGSTNDNA | 0.3 | −0.24 | −0.83 | −0.82 | ATLVT012XX2561 | 47495 | TGETAGSSNDNH | −1.13 | −1.82 | −0.29 | −0.95 |
| ATLVT012XX2516 | 47450 | TGESAGSTNDNH | 0.15 | 1.67 | −0.32 | 0.22 | ATLVT012XX2562 | 47496 | TGETAGSSNDNT | 1.96 | 0.34 | 0.18 | −1.26 |
| ATLVT012XX2517 | 47451 | TGESAGSTNDNT | −1.09 | 1.17 | −0.58 | −1.93 | ATLVT012XX2563 | 47497 | TGETAGSTNDNA | −2.34 | 1.11 | −1.16 | −1.71 |
| ATLVT012XX2518 | 47452 | TGESGAASNDNA | −1.41 | −0.82 | 1.33 | −3.59 | ATLVT012XX2564 | 47498 | TGETAGSTNDNH | −0.48 | 1.63 | 0.44 | −1.46 |
| ATLVT012XX2519 | 47453 | TGESGAASNDNH | −0.78 | −1.09 | −0.68 | −2.46 | ATLVT012XX2565 | 47499 | TGETAGSTNDNT | −0.81 | 2.41 | −1.18 | −0.51 |
| ATLVT012XX2520 | 47454 | TGESGAASNDNT | −0.54 | −2.25 | 0.8 | −3.55 | ATLVT012XX2566 | 47500 | TGETGAASNDNA | −3.06 | −1.63 | −1.98 | −0.64 |
| ATLVT012XX2521 | 47455 | TGESGAATNDNA | −2.65 | 0.29 | −1.79 | −1.64 | ATLVT012XX2567 | 47501 | TGETGAASNDNH | −2.26 | −1.4 | 0.03 | −1.68 |
| ATLVT012XX2522 | 47456 | TGESGAATNDNH | −2.5 | 1.77 | −1.93 | −1.78 | ATLVT012XX2568 | 47502 | TGETGAASNDNT | −2.33 | −1.72 | −3.18 | −2.49 |
| ATLVT012XX2523 | 47457 | TGESGAATNDNT | −2.01 | −0.16 | −2.13 | −2.68 | ATLVT012XX2569 | 47503 | TGETGAATNDNA | −2.62 | 2.32 | −2.17 | −1.81 |
| ATLVT012XX2524 | 47458 | TGESGASSNDNA | −0.13 | 0.89 | 0.34 | −0.5 | ATLVT012XX2570 | 47504 | TGETGAATNDNH | −3.5 | 1.31 | −0.1 | −1.38 |
| ATLVT012XX2525 | 47459 | TGESGASSNDNH | 0.23 | −0.91 | 0.55 | 0.01 | ATLVT012XX2571 | 47505 | TGETGAATNDNT | −2.68 | 1.88 | 0.14 | −2.72 |
| ATLVT012XX2526 | 47460 | TGESGASSNDNT | 0.15 | 0.75 | 0.1 | −2.02 | ATLVT012XX2572 | 47506 | TGETGASSNDNA | −1.38 | −1.09 | −1.76 | −2.55 |
| ATLVT012XX2527 | 47461 | TGESGASTNDNA | 0.98 | 0.62 | 0.94 | 0.93 | ATLVT012XX2573 | 47507 | TGETGASSNDNH | 1.82 | −1.72 | −1.87 | 0.4 |
| ATLVT012XX2528 | 47462 | TGESGASTNDNH | −1.08 | −0.7 | −1.67 | −1.19 | ATLVT012XX2574 | 47508 | TGETGASSNDNT | −0.37 | −1.03 | −1.77 | −3.1 |
| ATLVT012XX2529 | 47463 | TGESGASTNDNT | 0.45 | −1.23 | 0.21 | −1.97 | ATLVT012XX2575 | 47509 | TGETGASTNDNA | 0.6 | 1.62 | −0.6 | −2.15 |
| ATLVT012XX2530 | 47464 | TGESGGASNDNA | 2.39 | 2.97 | 0.76 | −2.24 | ATLVT012XX2576 | 47510 | TGETGASTNDNH | −1.67 | 4.08 | −0.06 | −0.54 |
| ATLVT012XX2577 | 47511 | TGETGASTNDNT | −2.38 | −0.42 | −1.74 | −2 | ATLVT012XX2623 | 47557 | TGQAGASTNDNA | 1.65 | 1.81 | 0.08 | −1.49 |
| ATLVT012XX2578 | 47512 | TGETGGASNDNA | −0.68 | 2.2 | −1.58 | 0.24 | ATLVT012XX2624 | 47558 | TGQAGASTNDNH | 1.32 | 1.09 | 0.22 | 1.09 |
| ATLVT012XX2579 | 47513 | TGETGGASNDNH | 2.44 | 2.25 | −0.98 | 1.39 | ATLVT012XX2625 | 47559 | TGQAGASTNDNT | 0.17 | 4.59 | −1.37 | −0.55 |
| ATLVT012XX2580 | 47514 | TGETGGASNDNT | 1.75 | 2.09 | −0.2 | −1.92 | ATLVT012XX2626 | 47560 | TGQAGGASNDNA | 0.47 | 0.36 | −0.35 | 1.39 |
| ATLVT012XX2581 | 47515 | TGETGGATNDNA | −1.3 | 1.34 | −0.66 | 0.79 | ATLVT012XX2627 | 47561 | TGQAGGASNDNH | 1.82 | 2 | 0.13 | 2.31 |
| ATLVT012XX2582 | 47516 | TGETGGATNDNH | 2.86 | 4.64 | 0.8 | 3.22 | ATLVT012XX2628 | 47562 | TGQAGGASNDNT | 2.39 | 0.53 | −1.2 | 0.22 |
| ATLVT012XX2583 | 47517 | TGETGGATNDNT | −0.08 | 1.44 | −0.01 | −1.03 | ATLVT012XX2629 | 47563 | TGQAGGATNDNA | 1.56 | 0.8 | −1.3 | 3.08 |
| ATLVT012XX2584 | 47518 | TGETGGSSNDNA | 1.4 | 3.92 | −0.4 | −1.07 | ATLVT012XX2630 | 47564 | TGQAGGATNDNH | 6.35 | 6.36 | −0.95 | 5.18 |
| ATLVT012XX2585 | 47519 | TGETGGSSNDNH | 1.29 | 0.81 | 0.43 | −1.14 | ATLVT012XX2631 | 47565 | TGQAGGATNDNT | 2.03 | 0.26 | −0.14 | 2.2 |
| ATLVT012XX2586 | 47520 | TGETGGSSNDNT | −0.21 | 1.99 | −0.14 | −1.16 | ATLVT012XX2632 | 47566 | TGQAGGSSNDNA | 1.73 | 1.5 | 0.65 | −1.21 |
| ATLVT012XX2587 | 47521 | TGETGGSTNDNA | −0.22 | 2.73 | −0.08 | 0.84 | ATLVT012XX2633 | 47567 | TGQAGGSSNDNH | −0.1 | −0.49 | −1.42 | 0.53 |
| ATLVT012XX2588 | 47522 | TGETGGSTNDNH | −0.02 | 0.66 | −1.16 | 0.55 | ATLVT012XX2634 | 47568 | TGQAGGSSNDNT | 0.9 | 0.02 | −1.56 | −1.54 |
| ATLVT012XX2589 | 47523 | TGETGGSTNDNT | 0.92 | 1.85 | −0.43 | −1.02 | ATLVT012XX2635 | 47569 | TGQAGGSTNDNA | 2.4 | 1.98 | −1.35 | 0.4 |
| ATLVT012XX2590 | 47524 | TGQAAAASNDNA | −2.51 | −1.77 | −0.23 | 1.25 | ATLVT012XX2636 | 47570 | TGQAGGSTNDNH | 2.83 | 4.9 | 0.03 | 1.1 |
| ATLVT012XX2591 | 47525 | TGQAAAASNDNH | −2.37 | −0.86 | 0.88 | 0.81 | ATLVT012XX2637 | 47571 | TGQAGGSTNDNT | 2.23 | 1.39 | −1.83 | −0.4 |
| ATLVT012XX2592 | 47526 | TGQAAAASNDNT | −2.93 | −2.05 | −1.22 | 2.74 | ATLVT012XX2638 | 47572 | TGQSAAASNDNA | −0.12 | 0.98 | −0.45 | 1.16 |
| ATLVT012XX2593 | 47527 | TGQAAAATNDNA | −0.93 | −0.29 | −1.1 | 0.12 | ATLVT012XX2639 | 47573 | TGQSAAASNDNH | −1.53 | −1.17 | 0.03 | 0.01 |
| ATLVT012XX2594 | 47528 | TGQAAAATNDNH | −1.67 | 1.59 | −1.01 | 1.52 | ATLVT012XX2640 | 47574 | TGQSAAASNDNT | −1.17 | 0.35 | 0.78 | 0.91 |
| ATLVT012XX2595 | 47529 | TGQAAAATNDNT | −1.91 | −0.75 | −0.48 | −1.66 | ATLVT012XX2641 | 47575 | TGQSAAATNDNA | −1.9 | 1.89 | 0.73 | −1.28 |
| ATLVT012XX2596 | 47530 | TGQAAASSNDNA | −2.79 | −2.46 | 0.18 | 0.19 | ATLVT012XX2642 | 47576 | TGQSAAATNDNH | −1.06 | −0.66 | −0.63 | −0.26 |
| ATLVT012XX2597 | 47531 | TGQAAASSNDNH | −1.54 | −1.96 | 0.37 | −3 | ATLVT012XX2643 | 47577 | TGQSAAATNDNT | −1.45 | −0.53 | −1.25 | −1.64 |
| ATLVT012XX2598 | 47532 | TGQAAASSNDNT | −1.65 | −2.13 | −0.5 | −1.44 | ATLVT012XX2644 | 47578 | TGQSAASSNDNA | −0.97 | 4.17 | 0.76 | −1.47 |
| ATLVT012XX2599 | 47533 | TGQAAASTNDNA | −0.12 | −1.37 | −1.17 | 0.66 | ATLVT012XX2645 | 47579 | TGQSAASSNDNH | −1.99 | 0.27 | 0.51 | −0.93 |
| ATLVT012XX2600 | 47534 | TGQAAASTNDNH | −0.59 | −1.75 | −1.97 | −0.09 | ATLVT012XX2646 | 47580 | TGQSAASSNDNT | −0.72 | 1.66 | −0.94 | 0.14 |
| ATLVT012XX2601 | 47535 | TGQAAASTNDNT | −0.35 | −2.49 | −1.14 | −0.73 | ATLVT012XX2647 | 47581 | TGQSAASTNDNA | −1.5 | 2.06 | −0.43 | −0.28 |
| ATLVT012XX2602 | 47536 | TGQAAGASNDNA | −2.27 | −2.62 | −2.5 | −2.44 | ATLVT012XX2648 | 47582 | TGQSAASTNDNH | −0.26 | 2.98 | −1.87 | 0.11 |
| ATLVT012XX2603 | 47537 | TGQAAGASNDNH | 0.06 | −0.42 | −1.77 | −0.43 | ATLVT012XX2649 | 47583 | TGQSAASTNDNT | −1.81 | 4.16 | −1.75 | 0.57 |
| ATLVT012XX2604 | 47538 | TGQAAGASNDNT | −2.62 | −1.99 | −1.94 | −0.65 | ATLVT012XX2650 | 47584 | TGQSAGASNDNA | −1.47 | 0.45 | −1.12 | −1.49 |
| ATLVT012XX2605 | 47539 | TGQAAGATNDNA | −2.8 | −2.24 | −3.16 | −2.8 | ATLVT012XX2651 | 47585 | TGQSAGASNDNH | 0.62 | 0.31 | 0.14 | −0.2 |
| ATLVT012XX2606 | 47540 | TGQAAGATNQNH | 3.35 | 2.98 | −0.57 | 2.14 | ATLVT012XX2652 | 47586 | TGQSAGASNDNT | 0.58 | −0.09 | −0.77 | −2.22 |
| ATLVT012XX2607 | 47541 | TGQAAGATNDNT | −1.09 | −0.93 | −2.69 | −1.68 | ATLVT012XX2653 | 47587 | TGQSAGATNDNA | −1.6 | 0.72 | −0.58 | −0.31 |
| ATLVT012XX2608 | 47542 | TGQAAGSSNDNA | −3.2 | −2.62 | −2.92 | −1.75 | ATLVT012XX2654 | 47588 | TGQSAGATNDNH | 1.14 | 2.25 | −1.01 | 2.82 |
| ATLVT012XX2609 | 47543 | TGQAAGSSNDNH | −1.9 | −2.59 | −1.31 | −0.6 | ATLVT012XX2655 | 47589 | TGQSAGATNDNT | −1.53 | 3.14 | −0.63 | −0.85 |
| ATLVT012XX2610 | 47544 | TGQAAGSSNDNT | −2.05 | −2.36 | −2.14 | −2.82 | ATLVT012XX2656 | 47590 | TGQSAGSSNDNA | 0.68 | 1.32 | −0.79 | −2.25 |
| ATLVT012XX2611 | 47545 | TGQAAGSTNDNA | −0.79 | −2.47 | −2.32 | −2.6 | ATLVT012XX2657 | 47591 | TGQSAGSSNDNH | 1.54 | 4.52 | −1.64 | 0.89 |
| ATLVT012XX2612 | 47546 | TGQAAGSTNDNH | −1.96 | 0.21 | −1.21 | −2.16 | ATLVT012XX2658 | 47592 | TGQSAGSSNDNT | 4.06 | 2.79 | −0.84 | −1.55 |
| ATLVT012XX2613 | 47547 | TGQAAGSTNDNT | −0.3 | −0.6 | −0.03 | −1.22 | ATLVT012XX2659 | 47593 | TGQSAGSTNDNA | −0.41 | 3.48 | 1.3 | −1.28 |
| ATLVT012XX2614 | 47548 | TGQAGAASNDNA | −0.93 | 2.46 | −0.54 | 2.22 | ATLVT012XX2660 | 47594 | TGQSAGSTNDNH | 0.32 | 2.91 | −1.39 | −1.92 |
| ATLVT012XX2615 | 47549 | TGQAGAASNDNH | 0.06 | 2.12 | −0.37 | 0.21 | ATLVT012XX2661 | 47595 | TGQSAGSTNDNT | −0.4 | 2.15 | −0.79 | −2.39 |
| ATLVT012XX2616 | 47550 | TGQAGAASNDNT | −0.1 | −1.34 | −2.39 | −0.66 | ATLVT012XX2662 | 47596 | TGQSGAASNDNA | −0.76 | 1.71 | −1.45 | 0.29 |
| ATLVT012XX2617 | 47551 | TGQAGAATNDNA | 0.99 | −0.38 | −2.6 | −1.29 | ATLVT012XX2663 | 47597 | TGQSGAASNDNH | −1.16 | 2.39 | −2.42 | −0.63 |
| ATLVT012XX2618 | 47552 | TGQAGAATNDNH | 0.38 | 2.38 | −3.44 | −1.39 | ATLVT012XX2664 | 47598 | TGQSGAASNDNT | 0.06 | 0.93 | −1.28 | −0.79 |
| ATLVT012XX2619 | 47553 | TGQAGAATNDNT | 0.51 | −0.08 | −2.45 | −0.61 | ATLVT012XX2665 | 47599 | TGQSGAATNDNA | −1.63 | 5.11 | −1.28 | −1.55 |
| ATLVT012XX2620 | 47554 | TGQAGASSNDNA | 0.93 | −0.95 | −1.4 | −2.58 | ATLVT012XX2666 | 47600 | TGQSGAATNDNH | −1.18 | 2.34 | −1.97 | −0.16 |
| ATLVT012XX2621 | 47555 | TGQAGASSNDNH | 2.65 | −1.18 | −0.44 | −2.29 | ATLVT012XX2667 | 47601 | TGQSGAATNDNT | −1.54 | 0.88 | −1.73 | −1.84 |
| ATLVT012XX2622 | 47556 | TGQAGASSNDNT | 3.2 | 0.41 | −0.6 | −1.99 | ATLVT012XX2668 | 47602 | TQSGASSNDNA | −0.22 | 2.49 | −0.36 | −0.12 |
| ATLVT012XX2669 | 47603 | TGQSGASSNDNH | 1.12 | −0.94 | 0.82 | 1.07 | ATLVT012XX2715 | 47649 | TGQTGAATNDNT | 3.85 | 3.32 | −2.6 | 1.56 |
| ATLVT01XXX2670 | 47604 | TGQSGASSNDNT | 1.66 | 0.31 | 0.76 | 0.96 | ATLVT012XX2716 | 47650 | TGQTGASSNDNA | 1.2 | −0.69 | 0.45 | −0.54 |
| ATLVT012XX2671 | 47605 | TGQSGASTNDNA | 0.58 | 2.92 | −0.24 | −0.23 | ATLVT012XX2717 | 47651 | TGQTGASSNDNH | −1.3 | 1.11 | 1.29 | −0.71 |
| ATLVT012XX2672 | 47606 | TGQSGASTNDNH | 0.34 | 3.02 | −0.8 | 1.5 | ATLVT012XX2718 | 47652 | TGQTGASSNDNT | −0.12 | −0.56 | 0.19 | 0.56 |
| ATLVT012XX2673 | 47607 | TGQSGASTNDNT | 2.59 | 1.74 | −0.17 | 0.86 | ATLVT012XX2719 | 47653 | TGQTGASTNDNA | 1.94 | 3.3 | 0.38 | 0.72 |
| ATLVT012XX2674 | 47608 | TGQSGGASNDNA | 2.27 | 4.73 | 0.76 | 0.71 | ATLVT012XX2720 | 47654 | TGQTGASTNDNH | 4.25 | 0.08 | −0.74 | −0.12 |
| ATLVT012XX2675 | 47609 | TGQSGGASNDNH | 2.83 | 3.19 | 0.64 | 2.99 | ATLVT012XX2721 | 47655 | TGQTGASTNDNT | 2.32 | 3.19 | −0.66 | −0.16 |
| ATLVT012XX2676 | 47610 | TGQSGGASNDNT | 2.05 | 3.29 | −0.64 | −0.59 | ATLVT012XX2722 | 47656 | TGQTGGASNDNA | 0.48 | 3.85 | 2.38 | 0.13 |
| ATLVT012XX2677 | 47611 | TGQSGGATNDNA | 1.79 | 3.24 | 1.28 | 1.81 | ATTVT012XX2723 | 47657 | TGQTGGASNDNH | 3.91 | 3.13 | 1.34 | 2.36 |
| ATLVT012XX2678 | 47612 | TGQSGGATNDNH | 6.13 | 6.98 | 0.66 | 3.84 | ATLVT012XX2724 | 47658 | TGQTGGASNDNT | 3.39 | 2.04 | 1.34 | 0.26 |
| ATLVT012XX2679 | 47613 | TGQSGGATNDNT | 2.86 | 5.22 | 0.17 | 0.9 | ATLVT012XX2725 | 47659 | TGQTGGATNDNA | 3 | 1.45 | −0.5 | 3.59 |
| ATLVT012XX2680 | 47614 | TGQSGGSSNDNA | 2.3 | 2.69 | −0.62 | −0.25 | ATLVT012XX2726 | 47660 | TGQTGGATNDNH | 7.35 | 7.38 | 1.16 | 4.18 |
| ATLVT012XX2681 | 47615 | TGQSGGSSNDNH | 2.31 | 0.6 | −0.74 | 0.81 | ATLVT012XX2727 | 47661 | TGQTGGATNDNT | 3.45 | 2.38 | −0.25 | 1.87 |
| ATLVT012XX2682 | 47616 | TGQSGGSSNDNT | 2.36 | 2.37 | −1.39 | −0.04 | ATLVT012XX2728 | 47662 | TGQTGGSSNDNA | 1.1 | 2.58 | 0.25 | 2.15 |
| ATLVT012XX2683 | 47617 | TGQSGGSTNDNA | 2.4 | 2.28 | 0.41 | 0.5 | ATLVT012XX2729 | 47663 | TGQTGGSSNDNH | 2.7 | 3.67 | 0.08 | 0.81 |
| ATLVT012XX2684 | 47618 | TGQSGGSTNDNH | 4.96 | 2.97 | 0.8 | 3.38 | ATLVT012XX2730 | 47664 | TGQTGGSSNDNT | 2.85 | 2.45 | 0.05 | 0.35 |
| ATLVT012XX2685 | 47619 | TGQSGGSTNDNT | 2.43 | 1.96 | −0.49 | −0.26 | ATLVT012XX2731 | 47665 | TGQTGGSTNDNA | 2.5 | 3.05 | 0.35 | 1.54 |
| ATLVT012XX2686 | 47620 | TGQTAAASNDNA | −0.56 | −1.72 | 0.59 | 1.91 | ATLVT012XX2732 | 47666 | TGQTGGSTNDNH | 2.78 | 4.11 | −0.23 | 3.37 |
| ATLVT012XX2687 | 47621 | TGQTAAASNDNH | 0.92 | −0.86 | −0.06 | −0.82 | ATLVT012XX2733 | 47667 | TGQTGGSTNDNT | 2.74 | 2.43 | 0.36 | 0.58 |
| ATLVT012XX2688 | 47622 | TGQTAAASNDNT | −0.37 | 0.37 | −1.16 | 1.43 | ATLVT012XX2734 | 47668 | TGTAAAASNDNA | 1.46 | 1.86 | −1.21 | −0.86 |
| ATLVT012XX2689 | 47623 | TGQTAAATNDNA | 0.59 | 1.06 | −0.84 | 2.18 | ATLVT012XX2735 | 47669 | TGTAAAASNDNH | 3.45 | 1.94 | −0.95 | −0.06 |
| ATLVT012XX2690 | 47624 | TGQTAAATNDNH | −0.34 | 3.66 | 0.28 | 2.42 | ATLVT012XX2736 | 47670 | TGTAAAASNDNT | 1.77 | 2.14 | −2.73 | 0.59 |
| ATLVT012XX2691 | 47625 | TGQTAAATNDNT | 0.4 | 0.74 | −0.19 | 0.99 | ATLVT012XX2737 | 47671 | TGTAAAATNDNA | 1.69 | 0.06 | −0.02 | 0.04 |
| ATLVT012XX2692 | 47626 | TGQTAASSNDNA | 2.21 | −1.3 | 0.59 | 0.92 | ATLVT012XX2738 | 47672 | TGTAAAATNDNH | 2.07 | 0.4 | −1.06 | −0.1 |
| ATLVT012XX2693 | 47627 | TGQTAASSNDNH | 0.8 | −1.38 | −1.35 | 0.28 | ATLVT012XX2739 | 47673 | TGTAAAATNDNT | 0.1 | −0.56 | −0.7 | −1.05 |
| ATLVT012XX2694 | 47628 | TGQTAASSNDNT | 3.83 | 0.03 | −0.18 | 0.71 | ATLVT012XX2740 | 47674 | TGTAAASSNDNA | 1.37 | 2.59 | −0.21 | −2.2 |
| ATLVT012XX2695 | 47629 | TGQTAASTNDNA | −1.54 | −0.12 | −0.36 | −0.59 | ATLVT012XX2741 | 47675 | TGTAAASSNDNH | 1.33 | 4.66 | 1.09 | −0.96 |
| ATLVT012XX2696 | 47630 | TGQTAASTNDNH | −1.22 | 1.99 | −0.49 | 1.3 | ATLVT012XX2742 | 47676 | TGTAAASSNDNT | 1.12 | 3.21 | 1.57 | −0.82 |
| ATLVT012XX2697 | 47631 | TGQTAASTNQNT | −1.57 | 2.89 | 0.78 | 0.77 | ATLVT012XX2743 | 47677 | TGTAAASTNDNA | −0.45 | 6.33 | 0.28 | −0.88 |
| ATLVT012XX2698 | 47632 | TGQTAGASNDNA | 0.32 | −0.63 | −1.91 | −0.22 | ATLVT012XX2744 | 47678 | TGTAAASTNDNH | 2.82 | 3.72 | 0.21 | 0.83 |
| ATLVT012XX2699 | 47633 | TGQTAGASNDNH | −0.11 | −0.79 | −2.03 | 3.12 | ATLVT012XX2745 | 47679 | TGTAAASTNDNT | 2.19 | 2.64 | −0.58 | −1.25 |
| ATLVT012XX2700 | 47634 | TGQTAGASNDNT | −0.93 | 2.21 | −0.15 | 0.21 | ATLVT012XX2746 | 47680 | TGTAAGASNDNA | 2.24 | 2.08 | −0.73 | −1.65 |
| ATLVT012XX2701 | 47635 | TGQTAGATNDNA | −2.67 | −1.24 | −1.26 | 0.5 | ATLVT012XX2747 | 47681 | TGTAAGASNDNH | 0.84 | 3.3 | 0.84 | 0.84 |
| ATLVT012XX2702 | 47636 | TGQTAGATNDNH | 0.48 | 1.68 | 0.02 | 1.56 | ATLVT012XX2748 | 47682 | TGTAAGASNDNT | 0.72 | 3.08 | −0.14 | −0.17 |
| ATLVT012XX2703 | 47637 | TGQTAGATNDNT | −1.04 | −0.17 | −0.47 | −0.44 | ATLVT012XX2749 | 47683 | TGTAAGATNDNA | 1.29 | 3.87 | −1.99 | −0.71 |
| ATLVT012XX2704 | 47638 | TGQTAGSSNDNA | −0.92 | 0.52 | −0.84 | −0.09 | ATLVT012XX2750 | 47684 | TGTAAGATNDNH | 5.59 | 5.58 | −0.44 | 3.12 |
| ATLVT012XX2705 | 47639 | TGQTAGSSNDNH | −1.84 | −1.33 | −1.84 | −1.5 | ATLVT012XX2751 | 47685 | TGTAAGATNDNT | 4.07 | 2.64 | −1.19 | 0.44 |
| ATLVT012XX2706 | 47640 | TGQTAGSSNDNT | −1.69 | 0.57 | −0.76 | −1.55 | ATLVT012XX2752 | 47686 | TGTAAGSSNDNA | 1.48 | 1.51 | 0.48 | −1.57 |
| ATLVT012XX2707 | 47641 | TGQTAGSTNDNA | −1.81 | −1.12 | −1.22 | −1.37 | ATLVT012XX2753 | 47687 | TGTAAGSSNDNH | −1.82 | −0.9 | −0.74 | −1.23 |
| ATLVT012XX2708 | 47642 | TGQTAGSTNDNH | 0.12 | 0.4 | −1.73 | −0.73 | ATLVT012XX2754 | 47688 | TGTAAGSSNDNT | −1.33 | 2.22 | −0.61 | −2.5 |
| ATLVT012XX2709 | 47643 | TGQTAGSTNDNT | −0.32 | 1.45 | −1.2 | 0.28 | ATLVT012XX2755 | 47689 | TGTAAGSTNDNA | −1.08 | 0.64 | −0.29 | −1.49 |
| ATLVT012XX2710 | 47644 | TGQTGAASNDNA | −1.07 | 0.41 | −0.98 | 0.02 | ATLVT012XX2756 | 47690 | TGTAAGSTNDNH | 0.4 | −0.02 | −1.57 | −0.46 |
| ATLVT012XX2711 | 47645 | TGQTGAASNDNH | −0.12 | 2.54 | 0.19 | −0.35 | ATLVT012XX2757 | 47691 | TGTAAGSTNDNT | −0.56 | 0.16 | −1.82 | −2.57 |
| ATLVT012XX2712 | 47646 | TGQTGAASNDNT | 0.28 | −0.54 | −0.18 | 1.31 | ATLVT012XX2758 | 47692 | TGTAGAASNDNA | −1.42 | −1.72 | −0.5 | −1.49 |
| ATLVT012XX2713 | 47647 | TGQTGAATNDNA | 1.46 | 1.7 | −2.21 | 0.73 | ATLVT012XX2759 | 47693 | TGTAGAASNDNG | 0.1 | −0.82 | −1.93 | −1.91 |
| ATLVT012XX2714 | 47648 | TGQTGAATNDNH | 1.25 | 0.58 | −1.41 | −1.37 | ATLVT012XX2760 | 47694 | TGTAGAASNDNT | −0.91 | −1.73 | −0.93 | −2.32 |
| ATLVT012XX2761 | 47695 | TGTAGAATNDNA | −2.07 | 0.14 | −2.73 | −2.03 | ATLVT012XX2807 | 47741 | TGTSGAASNDNH | −0.38 | 1.48 | −0.27 | −2.68 |
| ATLVT012XX2762 | 47696 | TGTAGAATNDNH | −1.03 | 1.57 | −2.27 | −0.61 | ATLVT012XX2808 | 47742 | TGTSGAASNDNT | −2.25 | −0.97 | −1.24 | −3.46 |
| ATLVT012XX2763 | 47697 | TGTAGAATNDNT | −2.94 | 0.23 | −3.59 | −3 | ATLVT012XX2809 | 47743 | TGTSGAATNDNA | −0.66 | 0.89 | −1.19 | −0.19 |
| ATLVT012XX2764 | 47698 | TGTAGASSNDNA | −2.62 | 0.11 | −1.61 | −1.23 | ATLVT012XX2810 | 47744 | TGTSGAATNDNH | −0.56 | 3.43 | 0 | 1.24 |
| ATLVT012XX2765 | 47699 | TGTAGASSNDNH | −2.49 | −0.91 | −1.94 | −1.41 | ATLVT012XX2811 | 47745 | TGTSGAATNDNT | −0.85 | 1.48 | −1.45 | −0.96 |
| ATLVT012XX2766 | 47700 | TGTAGASSNDNT | −0.99 | 0.77 | −1.2 | −2.36 | ATLVT012XX2812 | 47746 | TGTSGASSNDNA | 3.13 | −2.49 | 0.22 | −0.64 |
| ATLVT012XX2767 | 47701 | TGTAGASTNDNA | −1.57 | 0.92 | −1.08 | −0.06 | ATLVT012XX2813 | 47747 | TGTSGASSNDNH | 0.54 | −1.8 | 0.55 | 2.04 |
| ATLVT012XX2768 | 47702 | TGTAGASTNDNH | −3 | 0.39 | −0.69 | −0.86 | ATLVT012XX2814 | 47748 | TGTSGASSNDNT | 1.89 | −0.16 | 0.07 | −0.83 |
| ATLVT012XX2769 | 47703 | TGTAGASTNDNT | −2.15 | −1.38 | −1.39 | −0.32 | ATLVT012XX2815 | 47749 | TGTSGASTNDNA | −0.18 | −1.75 | 0.82 | 1.26 |
| ATLVT012XX2770 | 47704 | TGTAGGASNDNA | 2.96 | 2.91 | −1.6 | 0.16 | ATLVT012XX2816 | 47750 | TGTSGASTNDNH | −0.32 | −0.82 | −0.04 | −0.81 |
| ATLVT012XX2771 | 47705 | TGTAGGASNDNH | 2.99 | 3.84 | −0.9 | 0.46 | ATLVT012XX2817 | 47751 | TGTSGASTNDNT | −0.79 | −0.91 | 0.28 | 0.73 |
| ATLVT012XX2772 | 47706 | TGTAGGASNDNT | 2.18 | 3.58 | −1.87 | 0.16 | ATLVT012XX2818 | 47752 | TGTSGGASNDNA | 2.87 | 3.74 | −1.04 | −0.21 |
| ATLVT012XX2773 | 47707 | TGTAGGATNDNA | 0.93 | 2.09 | −0.51 | 1.2 | ATLVT012XX2819 | 47753 | TGTSGGASNDNH | 5.57 | 4.38 | 0.73 | 2.94 |
| ATLVT012XX2774 | 47708 | TGTAGGATNDNH | 5.89 | 7.95 | −0.6 | 3.67 | ATLVT012XX2820 | 47754 | TGTSGGASNDNT | 2.82 | 3.57 | −0.15 | −0.2 |
| ATLVT012XX2775 | 47709 | TGTAGGATNDNT | 2.5 | 4.79 | −1.69 | 0.33 | ATLVT012XX2821 | 47755 | TGTSGGATNDNA | 2.81 | 3.54 | 1.05 | 1.52 |
| ATLVT012XX2776 | 47710 | TGTAGGSSNDNA | 2.05 | 2.95 | −0.62 | 1.57 | ATLVT012XX2822 | 47756 | TGTSGGATNDNH | 7.78 | 9.35 | 1.64 | 4.16 |
| ATLVT012XX2777 | 47711 | TGTAGGSSNDNH | 2.15 | 1.71 | 1.02 | 1.14 | ATLVT012XX2823 | 47757 | TGTSGGATNDNT | 4.43 | 4.85 | 0.27 | 0.2 |
| ATLVT012XX2778 | 47712 | TGTAGGSSNDNT | 2.07 | 2.82 | −0.75 | 0.94 | ATLVT012XX2824 | 47758 | TGTSGGSSNDNA | 4.58 | 3.61 | 0.4 | 0.76 |
| ATLVT012XX2779 | 47713 | TGTAGGSTNDNA | 3.69 | 4.86 | −1.18 | 1.68 | ATLVT012XX2825 | 47759 | TGTSGGSSNDNH | 2.14 | 3.83 | −0.45 | 0.2 |
| ATLVT012XX2780 | 47714 | TGTAGGSTNDNH | 3.82 | 5.77 | −1.5 | 2.67 | ATLVT012XX2826 | 47760 | TGTSGGSSNDNT | 3.19 | 4.28 | −0.39 | −0.01 |
| ATLVT012XX2781 | 47715 | TGTAGGSTNDNT | 2.91 | 3.4 | −1.93 | 1.27 | ATLVT012XX2827 | 47761 | TGTSGGSTNDNA | 3.33 | 2.9 | 0.3 | 2.22 |
| ATLVT012XX2782 | 47716 | TGTSAAASNDNA | 0.5 | −0.84 | −0.06 | −1.98 | ATTVT012XX2828 | 47762 | TGTSGGSTNDNH | 2.05 | 4.57 | −0.35 | 3.1 |
| ATLVT012XX2783 | 47717 | TGTSAAASNDNH | 0.42 | 0.62 | −1.17 | −1.36 | ATLVT012XX2829 | 47763 | TGTSGGSTNDNT | 3.49 | 2.73 | 1.02 | 0.69 |
| ATLVT012XX2784 | 47718 | TGTSAAASNDNT | 2.68 | 0.94 | −0.21 | −2.09 | ATLVT012XX2830 | 47764 | TGTTAAASNDNA | −0.4 | 3.22 | 0.92 | 0.27 |
| ATLVT012XX2785 | 47719 | TGTSAAATNDNA | 0.85 | 0.72 | −0.35 | 0.62 | ATLVT012XX2831 | 47765 | TGTTAAASNDNH | 0.2 | −0.1 | 0.24 | −0.11 |
| ATLVT012XX2786 | 47720 | TGTSAAATNDNH | 0.53 | 1.94 | 0.25 | −0.11 | ATLVT012XX2832 | 47766 | TGTTAAASNDNT | −0.94 | 0.86 | −0.5 | 0.12 |
| ATLVT012XX2787 | 47721 | TGTSAAATNDNT | 1.89 | 0.07 | −0.19 | 0.37 | ATLVT012XX2833 | 47767 | TGTTAAATNDNA | 1.27 | 0.54 | −0.67 | −0.74 |
| ATLVT012XX2788 | 47722 | TGTSAASSNDNA | 0.03 | −0.19 | 0.22 | 1.92 | ATLVT012XX2834 | 47768 | TGTTAAATNDNH | 1.24 | −0.99 | −1.03 | 1.55 |
| ATLVT012XX2789 | 47723 | TGTSAASSNDNH | 1.23 | 0.47 | −0.13 | −0.14 | ATLVT012XX2835 | 47769 | TGTTAAATNDNT | 1.92 | 2.61 | 0.58 | −0.78 |
| ATLVT012XX2790 | 47724 | TGTSAASSNDNT | 1.33 | −0.32 | −0.65 | −2.04 | ATLVT012XX2836 | 47770 | TGTTAASSNDNA | 0.25 | −0.89 | −0.96 | 0.02 |
| ATLVT012XX2791 | 47725 | TGTSAASTNDNA | −0.35 | 4.99 | −0.91 | 0.59 | ATLVT012XX2837 | 47771 | TGTTAASSNDNH | −1.05 | 1.05 | −1.76 | −0.02 |
| ATLVT012XX2792 | 47726 | TGTSAASTNDNH | −0.55 | 3.82 | −0.88 | −0.72 | ATLVT012XX2838 | 47772 | TGTTAASSNDNT | 0.54 | 2.24 | −0.27 | −0.91 |
| ATLVT012XX2793 | 47727 | TGTSAASTNDNT | 1.12 | 4.81 | −1.48 | −0.53 | ATLVT012XX2839 | 47773 | TGTTAASTNDNA | 0.34 | −0.27 | −0.72 | 0.29 |
| ATLVT012XX2794 | 47728 | TGTSAGASNDNA | −0.51 | 0.2 | −0.59 | 0.49 | ATLVT012XX2840 | 47774 | TGTTAASTNDNH | 0.87 | 3.56 | −0.91 | 0.15 |
| ATLVT012XX2795 | 47729 | TGTSAGASNDNH | 0.52 | −0.77 | 0.35 | 1.78 | ATLVT012XX2841 | 47775 | TGTTAASTNDNT | 4.08 | 0.68 | −0.4 | −1.28 |
| ATLVT012XX2796 | 47730 | TGTSAGASNDNT | 0.46 | −0.91 | 0 | −0.37 | ATLVT012XX2842 | 47776 | TGTTAGASNDNA | −2.29 | 0.43 | 0.47 | −0.5 |
| ATLVT012XX2797 | 47731 | TGTSAGATNDNA | 1.52 | −0.18 | −0.37 | 1 | ATLVT012XX2843 | 47777 | TGTTAGASNDNH | −0.33 | 0.78 | −0.03 | 2.34 |
| ATLVT012XX2798 | 47732 | TGTSAGATNDNH | 6.23 | 3.39 | −0.23 | 3.74 | ATLVT012XX2844 | 47778 | TGTTAGASNDNT | 0.57 | 0.55 | −1.39 | 1 |
| ATLVT01XXX2799 | 47733 | TGTSAGATNDNT | 3.22 | 0.27 | 0.56 | 0.3 | ATLVT012XX2845 | 47779 | TGTTAGATNDNA | −1.65 | 1.26 | −1.46 | 1.88 |
| ATLVT012XX2800 | 47734 | TGTSAGSSNDNA | 0.21 | 2.2 | −0.41 | −0.48 | ATLVT012XX2846 | 47780 | TGTTAGATNDNH | 1.54 | 3.77 | −0.57 | 3.2 |
| ATLVT012XX2801 | 47735 | TGTSAGSSNDNH | −0.96 | −1 | −0.33 | −2.26 | ATLVT012XX2847 | 47781 | TGTTAGATNDNT | 0.51 | 1.62 | 0.38 | 1.23 |
| ATLVT012XX2802 | 47736 | TGTSAGSSNDNT | 0.77 | 1.57 | −0.42 | −2.11 | ATLVT012XX2848 | 47782 | TGTTAGSSNDNA | −1.88 | −0.77 | −1.29 | −1.49 |
| ATLVT012XX2803 | 47737 | TGTSAGSTNDNA | 0.22 | 2.32 | 0.16 | 0.4 | ATLVT012XX2849 | 47783 | TGTTAGSSNDNH | −0.37 | 1.02 | −1.77 | 0.37 |
| ATLVT012XX2804 | 47738 | TGTSAGSTNDNH | 2.29 | 1.53 | −0.46 | 1.99 | ATLVT012XX2850 | 47784 | TGTTAGSSNDNT | 1.05 | 0.3 | −0.51 | −0.39 |
| ATLVT012XX2805 | 47739 | TGTSAGSTNDNT | 2.12 | 1.1 | −0.76 | −0.32 | ATLVT012XX2851 | 47785 | TGTTAGSTNDNA | 0.02 | −0.78 | −1.12 | 0.75 |
| ATLVT012XX2806 | 47740 | TGTSGAASNDNA | −1.37 | −0.23 | −1.52 | −1.3 | ATLVT012XX2852 | 47786 | TGTTAGSTNDNH | −1.1 | −0.24 | −0.44 | 1.26 |
| ATLVT012XX2853 | 47787 | TGTTAGSTNDNT | 0.3 | 0.27 | −0.69 | 0.72 | ATTVT012XX2899 | 47833 | TSAAAGSTNDNA | 2.3 | −1.97 | −0.69 | −2.14 |
| ATLVT012XX2854 | 47788 | TGTTGAASNDNA | −1.83 | −1.85 | 1.21 | −1.53 | ATLVT012XX2900 | 47834 | TSAAAGSTNDNH | 0.42 | −3.26 | −0.34 | −0.63 |
| ATLVT012XX2855 | 47789 | TGTTGAASNDNH | −1.59 | −1.15 | −0.43 | −0.15 | ATLVT012XX2901 | 47835 | TSAAAGSTNDNT | 1.3 | −1.71 | −0.11 | −2.21 |
| ATLVT012XX2856 | 47790 | TGTTGAASNDNT | −2.94 | −2.48 | 0.32 | −1.59 | ATLVT012XX2902 | 47836 | TSAAGAASNDNA | −3.41 | −4.04 | −2.88 | −1.95 |
| ATLVT012XX2857 | 47791 | TGTTGAATNDNA | −0.18 | 1.8 | −0.94 | −1.48 | ATLVT012XX2903 | 47837 | TSAAGAASNDNH | −1.95 | −4.8 | −2.91 | −1.52 |
| ATLVT012XX2858 | 47792 | TGTTGAATNDNH | 1.37 | 0.16 | −1.39 | 0.21 | ATLVT012XX2904 | 47838 | TSAAGAASNDNT | −3.09 | −1.88 | −3.52 | −0.45 |
| ATLVT012XX2859 | 47793 | TGTTGAATNDNT | 0.63 | −0.88 | −0.67 | −0.86 | ATLVT012XX2905 | 47839 | TSAAGAATNDNA | −1.7 | | −2.65 | 0.11 |
| ATLVT012XX2860 | 47794 | TGTTGASSNDNA | −1.59 | −0.5 | 0.32 | −0.6 | ATLVT012XX2906 | 47840 | TSAAGAATNDNH | −1.08 | 0.85 | −2.57 | 0.12 |
| ATLVT012XX2861 | 47795 | TGTTGASSNDNH | 1.2 | 1 | −0.42 | −0.71 | ATLVT012XX2907 | 47841 | TSAAGAATNDNT | −2.34 | −1.91 | −2.69 | −2.85 |
| ATLVT012XX2862 | 47796 | TGTTGASSNDNT | −0.81 | −0.47 | −1.19 | −0.7 | ATLVT012XX2908 | 47842 | TSAAGASSNDNA | −2.37 | −0.98 | −1.12 | −2.23 |
| ATLVT012XX2863 | 47797 | TGTTGASTNDNA | −0.35 | −1.82 | −0.07 | −1.35 | ATLVT012XX2909 | 17843 | TSAAGASSNDNH | 1.37 | −5.49 | −1.59 | −1.89 |
| ATLVT012XX2864 | 47798 | TGTTGASTNDNH | −0.83 | −1.38 | 0.47 | 2.07 | ATLVT012XX2910 | 47844 | TSAAGASSNDNT | −3.12 | −4.55 | −1.02 | −1.38 |
| ATLVT012XX2865 | 47799 | TGTTGASTNDNT | 0.92 | −1.9 | −0.81 | −1.35 | ATLVT012XX2911 | 47845 | TSAAGASTNDNA | −1.87 | −4.21 | 0.21 | −0.74 |
| ATLVT012XX2866 | 47800 | TGTTGGASNDNA | 5.41 | 1.64 | 1.27 | 0.24 | ATLVT012XX2912 | 47846 | TSAAGASTNDNH | −2.95 | −4.66 | −1.6 | −2.74 |
| ATLVT012XX2867 | 47801 | TGTTGGASNDNH | 2.83 | 2.57 | −0.38 | 3.19 | ATLVT012XX2913 | 47847 | TSAAGASTNDNT | −0.45 | −4.11 | −0.34 | −2.47 |
| ATLVT012XX2868 | 47802 | TGTTGGASNDNT | 4.39 | 2.17 | 1.27 | −0.54 | ATLVT012XX2914 | 47848 | TSAAGGASNDNA | −0.43 | −0.83 | −3.05 | −1.72 |
| ATLVT012XX2869 | 47803 | TGTTGGATNDNA | 3.59 | 3.34 | 0.18 | 1.16 | ATTVT012XX2915 | 47849 | TSAAGGASNDNH | 0.32 | −1.92 | −0.86 | −1.24 |
| ATLVT012XX2870 | 47804 | TGTTGGATNDNH | 7.74 | 8.75 | 1.19 | 4.68 | ATLVT012XX2916 | 47850 | TSAAGGASNDNT | −0.71 | −0.43 | −4 | −2.07 |
| ATLVT012XX2871 | 47805 | TGTTGGATNDNT | 3.48 | 3.22 | −0.18 | 1.23 | ATLVT012XX2917 | 47851 | TSAAGGATNDNA | −1.32 | −1.46 | −1.76 | −0.51 |
| ATLVT012XX2872 | 47806 | TGTTGGSSNDNA | 1.33 | 2.75 | −0.13 | −0.23 | ATLVT012XX2918 | 47852 | TSAAGGATNDNH | 1.22 | −0.06 | −2.34 | 3.08 |
| ATLVT012XX2873 | 47807 | TGTTGGSSNDNH | 0.58 | 1.56 | 0.81 | −0.73 | ATLVT012XX2919 | 47853 | TSAAGGATNDNT | −1.26 | −2.63 | −1.87 | −1.02 |
| ATLVT012XX2874 | 47808 | TGTTGGSSNDNT | 2.47 | 3.32 | 0.07 | −0.02 | ATLVT012XX2920 | 47854 | TSAAGGSSNDNA | 0.9 | 0.05 | −1.65 | −1.48 |
| ATLVT012XX2875 | 47809 | TGTTGGSTNDNA | 2.76 | 2.97 | −0.48 | 1.45 | ATLVT012XX2921 | 47855 | TSAAGGSSNDNH | −2.6 | −2.94 | −0.77 | −2.04 |
| ATLVT012XX2876 | 47810 | TGTTGGSTNDNH | 3.73 | 2.41 | 0.93 | 1.96 | ATLVT012XX2922 | 47856 | TSAAGGSSNDNT | 0.06 | −2.99 | −2.9 | −2.94 |
| ATLVT012XX2877 | 47811 | TGTTGGSTNDNT | 2.68 | 3.18 | −0.05 | 1.17 | ATLVT012XX2923 | 47857 | TSAAGGSTNDNA | 0.96 | −1.89 | −2.24 | −2.16 |
| ATLVT012XX2878 | 47812 | TSAAAAASNDNA | 0.56 | −2.25 | −2.62 | −1.62 | ATLVT012XX2924 | 47858 | TSAAGGSTNDNH | 1.27 | −0.89 | −2.43 | −0.58 |
| ATLVT012XX2879 | 47813 | TSAAAAASNDNH | 0.33 | −2.78 | −1.68 | −3.46 | ATLVT012XX2925 | 47859 | TSAAGGSTNDNT | 1.88 | −1.52 | −2.19 | −0.96 |
| ATLVT012XX2880 | 47814 | TSAAAAASNDNT | 1.42 | 0.79 | −1.98 | −1.99 | ATLVT012XX2926 | 47860 | TSASAAASNDNA | 0.06 | 4.68 | −1.89 | 0.76 |
| ATLVT012XX2881 | 47815 | TSAAAAATNDNA | −0.83 | −0.02 | 0.9 | −0.91 | ATLVT012XX2927 | 47861 | TSASAAASNDNH | −1.01 | 2.99 | 1.15 | 0.14 |
| ATLVT012XX2882 | 47816 | TSAAAAATNDNH | −0.12 | −2.63 | 0.13 | −0.88 | ATTVT012XX2928 | 47862 | TSASAAASNDNT | −1.22 | 4.1 | −0.81 | −2.48 |
| ATLVT012XX2883 | 47817 | TSAAAAATNDNT | 0.42 | −2.09 | −0.55 | −1.54 | ATLVT012XX2929 | 47863 | TSASAAATNDNA | −1.27 | −0.22 | 1.89 | −0.79 |
| ATLVT012XX2884 | 47818 | TSAAAASSNDNA | −1.13 | −0.83 | −0.99 | −1.3 | ATLVT012XX2930 | 47864 | TSASAAATNDNH | −0.35 | 1.02 | 0.54 | 0.09 |
| ATLVT012XX2885 | 47819 | TSAAAASSNDNH | −1.11 | −0.28 | −1.43 | −1.35 | ATLVT012XX2931 | 47865 | TSASAAATNDNT | 1.73 | 1.57 | −0.05 | −0.94 |
| ATLVT012XX2886 | 47820 | TSAAAASSNDNT | 0.15 | 0.64 | 1.03 | −0.65 | ATLVT012XX2932 | 47866 | TSASAASSNDNA | 0.89 | 2.26 | −1.21 | −1.25 |
| ATLVT012XX2887 | 47821 | TSAAAASTNDNA | −2 | −2.21 | −0.76 | 1.16 | ATLVT012XX2933 | 47867 | TSASAASSNDNH | 1.41 | 1.62 | −0.62 | −1.43 |
| ATLVT012XX2888 | 47822 | TSAAAASTNDNH | −1.62 | −0.9 | −1.39 | −0.6 | ATLVT012XX2934 | 47868 | TSASAASSNDNT | 2.99 | 1.13 | −1.13 | −1.59 |
| ATLVT012XX2889 | 47823 | TSAAAASTNDNT | −2.2 | 0.92 | −0.73 | 1.2 | ATLVT012XX2935 | 47869 | TSASAASTNDNA | 2 | 2.56 | −0.36 | −0.08 |
| ATLVT012XX2890 | 47824 | TSAAAGASNDNA | −0.94 | 0.06 | −2.46 | −2.28 | ATTVT012XX2936 | 47870 | TSASAASTNDNH | 0.25 | 1.07 | 1.49 | −0.65 |
| ATLVT012XX2891 | 47825 | TSAAAGASNDNH | 0.88 | −0.59 | −2.65 | −2.41 | ATLVT012XX2937 | 47871 | TSASAASTNDNT | 1.08 | 2.23 | −0.1 | −2.41 |
| ATLVT012XX2892 | 47826 | TSAAAGASNDNT | 0.77 | −0.79 | −2 | −2.08 | ATLVT012XX2938 | 47872 | TSASAGASNDNA | −0.82 | 1.58 | −0.97 | 0.33 |
| ATLVT012XX2893 | 47827 | TSAAAGATNDNA | −0.35 | −0.39 | −1.59 | −0.33 | ATLVT012XX2939 | 47873 | TSASAGASNDNH | 0.21 | 3.86 | 0.56 | −1.58 |
| ATLVT012XX2894 | 47828 | TSAAAGATNDNH | 1.59 | 1.65 | −0.42 | 1.95 | ATLVT012XX2940 | 47874 | TSASAGASNDNT | 0.38 | 2.11 | −1.7 | −0.15 |
| ATLVT012XX2895 | 47829 | TSAAAGATNDNT | 1.27 | −1.16 | −0.81 | −0.95 | ATLVT012XX2941 | 47875 | TSASAGATNDNA | 1.31 | 0.68 | −1.22 | 0.98 |
| ATLVT012XX2896 | 47830 | TSAAAGSSNDNA | −1.37 | −3.23 | −0.55 | −2.85 | ATLVT012XX2942 | 47876 | TSASAGATNDNH | 0.28 | 3.33 | −0.82 | 0.33 |
| ATLVT012XX2897 | 47831 | TSAAAGSSNDNH | 1.11 | −2.02 | −2.11 | −2.06 | ATLVT012XX2943 | 47877 | TSASAGATNDNT | −0.23 | 0.14 | −1.53 | −0.69 |
| ATLVT012XX2898 | 47832 | TSAAAGSSNDNT | −0.13 | −2.27 | −1.31 | −2.67 | ATLVT012XX2944 | 47878 | TSASAGSSNDNA | −0.13 | 0.82 | −0.69 | −1.65 |
| ATLVT012XX2945 | 47879 | TSASAGSSNDNH | −1.29 | 2.03 | −0.05 | −1.56 | ATLVT012XX2991 | 47925 | TSATAGATNDNT | −0.03 | 1.64 | −0.98 | 0.23 |
| ATLVT012XX2946 | 47880 | TSASAGSSNDNT | 0.44 | 3.16 | −1.11 | −1.16 | ATLVT012XX2992 | 47926 | TSATAGSSNDNA | 0.38 | 0.73 | −0.18 | −3.22 |
| ATLVT012XX2947 | 47881 | TSASAGSTNDNA | −1.01 | 2.82 | −1.43 | 0.3 | ATLVT012XX2993 | 47927 | TSATAGSSNDNH | −0.56 | −2.08 | −0.44 | −0.91 |
| ATLVT012XX2948 | 47882 | TSASAGSTNDNH | −0.09 | 0.36 | −0.34 | −1.75 | ATLVT012XX2994 | 47928 | TSATAGSSNDNT | 0.35 | 1.01 | 0.09 | −1.33 |
| ATLVT012XX2949 | 47883 | TSASAGSTNDNT | 0.62 | 2.41 | −1.67 | −1.19 | ATLVT012XX2995 | 47929 | TSATAGSTNDNA | −2.21 | −1.27 | −1.02 | 0.07 |
| ATLVT012XX2950 | 47884 | TSASGAASNDNA | 0 | 0.1 | −1.49 | −0.44 | ATLVT012XX2996 | 47930 | TSATAGSTNDNH | −2.21 | 0.65 | −1.18 | −0.34 |
| ATLVT012XX2951 | 47885 | TSASGAASNDNH | −1.65 | −2.21 | −0.43 | −0.01 | ATLVT012XX2997 | 47931 | TSATAGSTNDNT | −1.61 | 0.34 | −0.1 | 0.17 |
| ATLVT012XX2952 | 47886 | TSASGAASNDNT | −0.63 | −1.47 | −1.54 | −2.14 | ATLVT012XX2998 | 47932 | TSATGAASNDNA | −1.21 | −2.64 | −1.54 | −0.86 |
| ATLVT012XX2953 | 47887 | TSASGAATNDNA | −2.21 | 5.25 | −2.05 | −1.77 | ATLVT012XX2999 | 47933 | TSATGAASNDNH | 0.4 | −1.79 | 0.28 | −1.35 |
| ATLVT012XX2954 | 47888 | TSASGAATNDNH | −2.37 | 1.91 | 0.66 | −1.1 | ATLVT012XX3000 | 47934 | TSATGAASNDNT | −1.31 | −2.68 | −1.44 | −2.71 |
| ATLVT012XX2955 | 47889 | TSASGAATNDNT | −1.7 | 2.2 | −0.2 | −2.09 | ATLVT012XX3001 | 47935 | TSATGAATNDNA | −1.25 | −1.08 | −0.96 | −1.79 |
| ATLVT012XX2956 | 47890 | TSASGASSNDNA | 1.41 | −2.84 | −0.23 | −0.73 | ATLVT012XX3002 | 47936 | TSATGAATNDNH | 0.06 | −1.14 | 0.37 | 1.13 |
| ATLVT012XX2957 | 47891 | TSASGASSNDNH | 1.42 | −3.39 | −2.1 | −3.85 | ATLVT012XX3003 | 47937 | TSATGAATNDNT | 1.48 | −0.75 | −1.33 | −0.98 |
| ATLVT012XX2958 | 47892 | TSASGASSNDNT | 1.4 | −2.24 | −1.64 | −1.17 | ATLVT012XX3004 | 47938 | TSATGASSNDNA | −2.14 | −1.64 | −1.12 | −0.21 |
| ATLVT012XX2959 | 47893 | TSASGASTNDNA | −1.29 | −0.83 | 0.61 | −1.66 | ATLVT012XX3005 | 47939 | TSATGASSNDNH | −2.62 | −0.17 | 0.13 | −0.75 |
| ATLVT012XX2960 | 47894 | TSASGASTNDNH | −1.4 | −2.98 | 0.46 | 0.29 | ATLVT012XX3006 | 47940 | TSATGASSNDNT | −0.28 | −1.61 | 0.09 | −1.02 |
| ATLVT012XX2961 | 47895 | TSASGASTNDNT | 1.74 | −2.08 | −1.11 | −1.25 | ATLVT012XX3007 | 47941 | TSATGASTNDNA | −0.97 | −1 | −0.43 | 0.43 |
| ATLVT012XX2962 | 47896 | TSASGGASNDNA | 1.32 | 2.19 | −1.44 | −0.14 | ATLVT012XX3008 | 47942 | TSATGASTNDNH | −0.69 | −1.6 | −1.62 | 0.51 |
| ATLVT012XX2963 | 47897 | TSASGGASNDNH | 2.4 | −0.33 | −1.1 | 0.85 | ATLVT012XX3009 | 47943 | TSATGASTNDNT | −0.6 | 0.64 | −0.27 | −1.11 |
| ATLVT012XX2964 | 47898 | TSASGGASNDNT | 3.45 | 0.61 | −0.98 | −1.14 | ATLVT012XX3010 | 47944 | TSATGGASNDNA | 1.08 | −0.01 | −1.41 | 0.43 |
| ATLVT012XX2965 | 47899 | TSASGGATNDNA | 0.64 | 1.18 | 0.5 | 2.21 | ATLVT012XX3011 | 47945 | TSATGGASNDNH | 1.24 | 0.98 | 0 | 0.87 |
| ATLVT012XX2966 | 47900 | TSASGGATNDNH | 4.02 | 4 | 1.52 | 2.86 | ATLVT012XX3012 | 47946 | TSATGGASNDNT | 2.25 | 2.34 | −0.48 | 2.88 |
| ATLVT012XX2967 | 47901 | TSASGGATNDNT | 3.37 | 1.15 | −0.89 | 0.35 | ATLVT012XX3013 | 47947 | TSATGGATNDNA | 1.52 | 0.14 | −0.85 | −0.41 |
| ATLVT012XX2968 | 47902 | TSASGGSSNDNA | 1.59 | 0.23 | −1.17 | −1.61 | ATLVT012XX3014 | 47948 | TSATGGATNDNH | 4.02 | 1.85 | 0.09 | 2.31 |
| ATLVT012XX2969 | 47903 | TSASGGSSNDNH | 0.15 | −2.02 | −0.24 | −0.88 | ATLVT012XX3015 | 47949 | TSATGGATNDNT | 2.85 | 1.9 | −0.8 | −1.14 |
| ATLVT012XX2970 | 47904 | TSASGGSSNDNT | 2.2 | −1.01 | 0.65 | −0.95 | ATLVT012XX3016 | 47950 | TSATGGSSNDNA | −0.2 | 3.15 | 0.3 | 0.77 |
| ATLVT012XX2971 | 47905 | TSASGGSTNDNA | 1.56 | 0.07 | 0.44 | 0.43 | ATLVT012XX3017 | 47951 | TSATGGSSNDNH | 2.26 | 1.03 | −0.23 | −0.21 |
| ATLVT012XX2972 | 47906 | TSASGGSTNDNH | 0.95 | 0.77 | −0.74 | 1.6 | ATLVT012XX3018 | 47952 | TSATGGSSNDNT | 1.34 | 1.53 | 0.78 | 0.45 |
| ATLVT012XX2973 | 47907 | TSASGGSTNDNT | 2.78 | 0.62 | 1.3 | −0.46 | ATLVT012XX3019 | 47953 | TSATGGSTNDNA | 2.21 | 0.9 | 0.83 | 0.86 |
| ATLVT012XX2974 | 47908 | TSATAAASNDNA | 0.64 | −1.21 | 0.58 | 0.01 | ATLVT012XX3020 | 47954 | TSATGGSTNQNH | 2.83 | −0.08 | 0.96 | 1.19 |
| ATLVT012XX2975 | 47909 | TSATAAASNDNH | 1.87 | 0.61 | −0.64 | −0.93 | ATLVT012XX3021 | 47955 | TSATGGSTNDNT | 2.89 | 1.5 | 0.05 | −1.21 |
| ATLVT012XX2976 | 47910 | TSATAAASNDNT | 3.72 | −1.73 | 1.24 | −1.12 | ATLVT012XX3022 | 47956 | TSEAAAASNDNA | −0.25 | 0.81 | 0 | 0.91 |
| ATLVT012XX2977 | 47911 | TSATAAATNDNA | 0.11 | −1.39 | −2.1 | −0.78 | ATLVT012XX3023 | 47957 | TSEAAAASNDNH | 1.27 | −0.38 | −0.15 | −1.18 |
| ATLVT012XX2978 | 47912 | TSATAAATNDNH | 0.48 | −0.29 | −0.38 | −0.4 | ATLVT012XX3024 | 47958 | TSEAAAASNDNT | −0.98 | 1.6 | −0.72 | −1.37 |
| ATLVT012XX2979 | 47913 | TSATAAATNDNT | 0.99 | −0.81 | −1.49 | −2.77 | ATLVT012XX3025 | 47959 | TSEAAAATNDNA | −1.08 | 6.76 | −0.32 | 1 |
| ATLVT012XX2980 | 47914 | TSATAASSNDNA | −0.9 | −2.77 | −0.76 | 0 | ATLVT012XX3026 | 47960 | TSEAAAATNDNH | 0.47 | 2.95 | −0.42 | 0.58 |
| ATLVT012XX2981 | 47915 | TSATAASSNDNH | −0.03 | −2.58 | −1.57 | −0.15 | ATLVT012XX3027 | 47961 | TSEAAAATNDNT | −1.98 | 0.92 | −0.01 | −0.52 |
| ATLVT012XX2982 | 47916 | TSATAASSNDNT | −0.67 | −2.13 | −1.14 | −1.78 | ATLVT012XX3028 | 47962 | TSEAAASSNDNA | −1.01 | −1 | −2.01 | −1.09 |
| ATLVT012XX2983 | 47917 | TSATAASTNDNA | −2.03 | −1.83 | −0.1 | −0.5 | ATLVT012XX3029 | 47963 | TSEAAASSNDNH | −2.42 | −1.72 | −1.28 | −4 |
| ATLVT012XX2984 | 47918 | TSATAASTNDNH | −2.72 | 0.21 | −0.51 | −2.02 | ATLVT012XX3030 | 47964 | TSEAAASSNDNT | 1.4 | −0.91 | −0.75 | −1.95 |
| ATLVT012XX2985 | 47919 | TSATAASTNDNT | −1.54 | −0.81 | 0.21 | −1.04 | ATLVT012XX3031 | 47965 | TSEAAASTNDNA | −2.04 | −0.01 | 0.1 | −1.38 |
| ATLVT012XX2986 | 47920 | TSATAGASNDNA | −2.09 | −0.11 | −1.82 | −0.92 | ATLVT012XX3032 | 47966 | TSEAAASTNDNH | −3.39 | −1.7 | −2.36 | −1.37 |
| ATLVT012XX2987 | 47921 | TSATAGASNDNH | −2.24 | 0.47 | −2.53 | −2.22 | ATLVT012XX3033 | 47967 | TSEAAASTNDNT | −1.65 | −0.31 | 0.18 | −1.67 |
| ATLVT012XX2988 | 47922 | TSATAGASNDNT | −1.18 | 0.44 | −0.94 | −1.08 | ATLVT012XX3034 | 47968 | TSEAAGASNDNA | −1.65 | 3.83 | −2.27 | −3.26 |
| ATLVT012XX2989 | 47923 | TSATAGATNDNA | −2.47 | 1.8 | −1.26 | 0.14 | ATLVT012XX3035 | 47969 | TSEAAGASNDNH | −2.5 | 0.15 | −2.38 | −2.93 |
| ATLVT012XX2990 | 47924 | TSATAGATNDNH | −0.55 | 1.09 | −1.42 | −0.19 | ATLVT012XX3036 | 47970 | TSEAAGASNDNT | −1.82 | −0.27 | −2.777 | −3.36 |
| ATLVT012XX3037 | 47971 | TSEAAGATNDNA | −0.74 | −0.3 | −2.9 | −0.41 | ATLVT012XX3083 | 48017 | TSESAGASNDNH | 0.61 | 1.31 | −1.39 | −1.95 |
| ATLVT012XX3038 | 47972 | TSEAAGATNDNH | −0.48 | 1.48 | −3.15 | −2.34 | ATLVT012XX3084 | 48018 | TSESAGASNDNT | 1.17 | −1.49 | −1.06 | −1.3 |
| ATLVT012XX3039 | 47973 | TSEAAGATNDNT | 0.01 | −0.68 | −0.71 | −2.89 | ATLVT012XX3085 | 48019 | TSESAGATNDNA | −2.22 | −0.66 | −0.83 | −1.54 |
| ATLVT012XX3040 | 47974 | TSEAAGSSNDNA | −3.14 | −1.37 | −1.97 | −2.52 | ATLVT012XX3086 | 48020 | TSESAGATNDNH | 0.74 | 4.13 | −1.25 | −0.83 |
| ATLVT012XX3041 | 47975 | TSEAAGSSNDNH | −3.68 | −2.03 | −2.43 | −2.49 | ATLVT012XX3087 | 48021 | TSESAGATNDNT | −1.21 | −0.4 | −2.15 | −2.88 |
| ATLVT012XX3042 | 47976 | TSEAAGSSNDNT | −3.17 | −1.76 | −1.87 | 0.32 | ATLVT012XX3088 | 48022 | TSESAGSSNDNA | 1.12 | 1.02 | −1.74 | 0.92 |
| ATLVT012XX3043 | 47977 | TSEAAGSTNDNA | −3.28 | 0.76 | −1.02 | −3.6 | ATLVT012XX3089 | 48023 | TSESAGSSNDNH | 1.46 | 3.9 | −0.76 | −0.37 |
| ATLVT012XX3044 | 47978 | TSEAAGSTNDNH | −2.92 | 1.86 | −2.24 | −3.66 | ATLVT012XX3090 | 48024 | TSESAGSSNDNT | 1.86 | −0.45 | −1.58 | −1.29 |
| ATLVT012XX3045 | 47979 | TSEAAGSTNDNT | −2.47 | −0.53 | −1.65 | −3.62 | ATLVT012XX3091 | 48025 | TSESAGSTNDNA | 1.8 | −0.83 | 0.01 | −2.23 |
| ATLVT012XX3046 | 47980 | TSEAGAASNDNA | −0.62 | −1.27 | −2.77 | −2.32 | ATLVT012XX3092 | 48026 | TSESAGSTNDNH | 0.64 | −1.2 | −0.78 | −0.72 |
| ATLVT012XX3047 | 47981 | TSEAGAASNDNH | −0.98 | −1.69 | −1.84 | −2.72 | ATLVT012XX3093 | 48027 | TSESAGSTNDNT | 0.64 | −1.07 | −0.71 | −1.57 |
| ATLVT012XX3048 | 47982 | TSEAGAASNDNT | −3.24 | −2.23 | −0.95 | −1.64 | ATLVT012XX3094 | 48028 | TSESGAASNDNA | 0.2 | −1.91 | −0.72 | −1.76 |
| ATLVT012XX3049 | 47983 | TSEAGAATNDNA | −3.18 | −0.16 | −2.66 | −2.77 | ATLVT012XX3095 | 48029 | TSESGAASNDNH | 1.47 | 0.88 | −1.78 | −0.77 |
| ATLVT012XX3050 | 47984 | TSEAGAATNDNH | 0.02 | 3.48 | −2.99 | −0.18 | ATLVT012XX3096 | 48030 | TSESGAASNDNT | 0.91 | −1.78 | −0.85 | −2.21 |
| ATLVT012XX3051 | 47985 | TSEAGAATNDNT | −2.08 | 0.95 | −3.23 | −1.61 | ATLVT012XX3097 | 48031 | TSESGAATNDNA | −0.93 | −1.44 | −0.26 | −0.87 |
| ATLVT012XX3052 | 47986 | TSEAGASSNDNA | −2.34 | −1.02 | −0.62 | −1.4 | ATLVT012XX3098 | 48032 | TSESGAATNDNH | 1.14 | −1.61 | −1.58 | −1.2 |
| ATLVT012XX3053 | 47987 | TSEAGASSNDNH | −3.52 | −1.4 | −1.08 | −2.48 | ATLVT012XX3099 | 48033 | TSESGAATNDNT | −0.41 | −2.19 | −0.46 | −0.56 |
| ATLVT012XX3054 | 47988 | TSEAGASSNDNT | −2.35 | −2.34 | −0.35 | −3.17 | ATLVT012XX3100 | 48034 | TSESGASSNDNA | −1.02 | 2.64 | −0.22 | −2.5 |
| ATLVT012XX3055 | 47989 | TSEAGASTNDNA | −3.8 | −1.1 | −0.53 | −1.83 | ATLVT012XX3101 | 48035 | TSESGASSNDNH | −1.76 | −0.19 | −0.08 | −3.67 |
| ATLVT012XX3056 | 47990 | TSEAGASTNDNH | −3.92 | 3.02 | −0.77 | −2.18 | ATLVT012XX3102 | 48036 | TSESGASSNDNT | −1.62 | 2.17 | 0.01 | −2.12 |
| ATLVT012XX3057 | 47991 | TSEAGASTNDNT | −2.69 | −0.58 | −1.44 | −0.75 | ATLVT012XX3103 | 48037 | TSESGASTNDNA | 0.81 | −2.19 | −0.74 | 0.7 |
| ATLVT012XX3058 | 47992 | TSEAGGASNDNA | 0.42 | 0.59 | 0.84 | 0.01 | ATLVT012XX3104 | 48038 | TSESGASTNDNH | −1.47 | −0.78 | 0.13 | −0.92 |
| ATLVT01XXX3059 | 47993 | TSEAGGASNDNH | 2.16 | 4.1 | −0.88 | −0.89 | ATLVT012XX3105 | 48039 | TSESGASTNDNT | 1.38 | −0.26 | 0.52 | −1.44 |
| ATLVT012XX3060 | 47994 | TSEAGGASNDNT | 1.21 | 0.26 | −0.89 | −0.3 | ATLVT012XX3106 | 48040 | TSESGGASNDNA | 0.58 | −0.89 | −0.45 | −0.93 |
| ATLVT012XX3061 | 47995 | TSEAGGATNDNA | −0.24 | −0.41 | 2.19 | 0.29 | ATLVT012XX3107 | 48041 | TSESGGASNDNH | 0.83 | 1.81 | 0.84 | 0.4 |
| ATLVT012XX3062 | 47996 | TSEAGGATNDNH | 3.94 | 2.5 | 1.17 | 2.63 | ATLVT012XX3108 | 48042 | TSESGGASNDNT | 1.26 | 1.7 | 0.49 | −1.82 |
| ATLVT012XX3063 | 47997 | TSEAGGATNDNT | 0.84 | 1.01 | 1.01 | 0.4 | ATLVT012XX3109 | 48043 | TSESGGATNDNA | 0.36 | 2.3 | −0.87 | −0.16 |
| ATLVT012XX3064 | 47998 | TSEAGGSSNDNA | 0.55 | 0.95 | −2.04 | −0.42 | ATLVT012XX3110 | 48044 | TSESGGATNDNH | 2.79 | 1.18 | 0.21 | 1.01 |
| ATLVT012XX3065 | 47999 | TSEAGGSSNDNH | 0.28 | −0.42 | 0.24 | −1.66 | ATLVT012XX3111 | 48045 | TSESGGATNDNT | 3.07 | −0.82 | −0.81 | 0.18 |
| ATLVT012XX3066 | 48000 | TSEAGGSSNDNT | 0.89 | −0.19 | −0.48 | 0 | ATLVT012XX3112 | 48046 | TSESGGSSNDNA | 2.41 | 0.85 | −0.68 | −0.25 |
| ATLVT012XX3067 | 48001 | TSEAGGSTNDNA | 2.57 | 2.02 | 0.27 | 0.05 | ATLVT012XX3113 | 48047 | TSESGGSSNDNH | 0.73 | −1.19 | 0.9 | −1.59 |
| ATLVT012XX3068 | 48002 | TSEAGGSTNDNH | 0.68 | −0.39 | −0.67 | −0.55 | ATLVT012XX3114 | 48048 | TSESGGSSNDNT | 2.13 | 1.55 | 0.33 | 0.26 |
| ATLVT012XX3069 | 48003 | TSEAGGSTNDNT | 2.69 | 0.83 | −0.61 | −0.71 | ATLVT012XX3115 | 48049 | TSESGGSTNDNA | 2.89 | 0.28 | −0.48 | −0.34 |
| ATLVT012XX3070 | 48004 | TSESAAASNDNA | 1 | −1.03 | −1.76 | −2.16 | ATLVT012XX3116 | 48050 | TSESGGSTNDNH | 1.37 | 0.92 | −0.29 | 0.51 |
| ATLVT012XX3071 | 48005 | TSESAAASNDNH | 1.09 | 2.45 | −1.93 | −3.85 | ATLVT012XX3117 | 48051 | TSESGGSTNDNT | 1.56 | 1.93 | −0.27 | −1.65 |
| ATLVT012XX3072 | 48006 | TSESAAASNDNT | 3.14 | −0.51 | −0.94 | −2.79 | ATLVT012XX3118 | 48052 | TSETAAASNDNA | −0.54 | −0.93 | −1.2 | −1.28 |
| ATLVT012XX3073 | 48007 | TSESAAATNDNA | 0.1 | 0.5 | −0.53 | −2.51 | ATLVT012XX3119 | 48053 | TSETAAASNDNH | −0.72 | 0.56 | −1.42 | −2.67 |
| ATLVT012XX3074 | 48008 | TSESAAATNDNH | −1.29 | −0.36 | −1.14 | 0.26 | ATLVT012XX3120 | 48054 | TSETAAASNDNT | −0.5 | 1.08 | −1.57 | −0.3 |
| ATLVT012XX3075 | 48009 | TSESAAATNDNT | −1.71 | 0.55 | −1.73 | −3.97 | ATLVT012XX3121 | 48055 | TSETAAATNDNA | 0.75 | 2.84 | −0.22 | 0.6 |
| ATLVT012XX3076 | 48010 | TSESAASSNDNA | −0.46 | −0.87 | −0.9 | −0.66 | ATLVT012XX3122 | 48056 | TSETAAATNDNH | 1.59 | 0.9 | −0.99 | 0.39 |
| ATLVT012XX3077 | 48011 | TSESAASSNDNH | 1.4 | 1.44 | −0.26 | −2.67 | ATLVT012XX3123 | 48057 | TSETAAATNDNT | 1.48 | 3 | −1.53 | −1.74 |
| ATLVT012XX3078 | 48012 | TSESAASSNDNT | 1.06 | 0.16 | −1.09 | −3.3 | ATLVT012XX3124 | 48058 | TSETAASSNDNA | −1.5 | 1.69 | −0.21 | 0.24 |
| ATLVT012XX3079 | 48013 | TSESAASTNDNA | −0.17 | −0.72 | −1.89 | −2.09 | ATLVT012XX3125 | 48059 | TSETAASSNDNH | −2.72 | 1.49 | −1.04 | −0.1 |
| ATLVT012XX3080 | 48014 | TSESAASTNDNH | −2.37 | 0.53 | −1.59 | −2.55 | ATLVT012XX3126 | 48060 | TSETAASSNDNT | −2.83 | −1.12 | −1.14 | −0.11 |
| ATLVT012XX3081 | 48015 | TSESAASTNDNT | −0.13 | −0.63 | 0.3 | −1.96 | ATLVT012XX3127 | 48061 | TSETAASTNDNA | 1.06 | −1.56 | 0.9 | −0.93 |
| ATLVT012XX3082 | 48016 | TSESAGASNDNA | 0.8 | 0.08 | −2.42 | −1.51 | ATLVT012XX3128 | 48062 | TSETAASTNDNH | −0.34 | −1.41 | −0.38 | −1.91 |
| ATLVT012XX3129 | 48063 | TSETAASTNDNT | 0.76 | −2.05 | −0.71 | 0.15 | ATLVT012XX3175 | 48109 | TSQAAASTNDNA | −1.3 | −1.46 | −1.32 | −1.45 |
| ATLVT01XXX3130 | 48064 | TSETAGASNDNA | −0.47 | 1.28 | −1.44 | −0.57 | ATLVT012XX3176 | 48110 | TSQAAASTNDNH | −0.2 | −1.5 | −0.33 | −0.77 |
| ATLVT012XX3131 | 48065 | TSETAGASNDNH | −0.08 | 0.32 | −1.7 | 1.08 | ATLVT012XX3177 | 48111 | TSQAAASTNDNT | −0.81 | −1.16 | −1.49 | −2.34 |
| ATLVT012XX3132 | 48066 | TSETAGASNDNT | 0.09 | 0.98 | −0.65 | −1.32 | ATLVT012XX3178 | 48112 | TSQAAGASNDNA | 1.95 | 2.69 | −1.88 | −1.04 |
| ATLVT012XX3133 | 48067 | TSETAGATNDNA | −0.37 | 3.8 | −1.29 | 0.48 | ATLVT012XX3179 | 48113 | TSQAAGASNDNH | 1.45 | −0.82 | −1.26 | −3.52 |
| ATLVT012XX3134 | 48068 | TSETAGATNDNH | −0.22 | 0.44 | 0.03 | −0.22 | ATLVT012XX3180 | 48114 | TSQAAGASNDNT | 1.07 | 2 | −3.1 | −3.27 |
| ATLVT012XX3135 | 48069 | TSETAGATNDNT | −0.53 | 2.3 | 0.3 | −1.74 | ATLVT012XX3181 | 48115 | TSQAAGATNDNA | −0.25 | 3.29 | −3.15 | −2.42 |
| ATLVT012XX3136 | 48070 | TSETAGSSNDNA | −0.32 | −0.42 | 0.62 | −2.7 | ATLVT012XX3182 | 48116 | TSQAAGATNDNH | 1.07 | 3.37 | −3.28 | −1.72 |
| ATLVT012XX3137 | 48071 | TSETAGSSNDNH | −1.14 | 2.59 | −0.63 | −1.41 | ATLVT012XX3183 | 48117 | TSQAAGATNDNT | 1.19 | 1.45 | −3.57 | −1.89 |
| ATLVT012XX3138 | 48072 | TSETAGSSNDNT | −0.96 | −0.79 | −0.01 | −2.25 | ATLVT012XX3184 | 48118 | TSQAAGSSNDNA | −3.18 | −1.12 | −1.21 | −2.1 |
| ATLVT012XX3139 | 48073 | TSETAGSTNDNA | 0.99 | 0.99 | −0.32 | −2.05 | ATLVT012XX3185 | 48119 | TSQAAGSSNDNH | −2.94 | −1.67 | −3.15 | −3.45 |
| ATLVT012XX3140 | 48074 | TSETAGSTNDNH | 0.35 | −1.78 | −1.03 | −1.35 | ATLVT012XX3186 | 48120 | TSQAAGSSNDNT | −1.28 | −0.65 | −2.94 | −2.91 |
| ATLVT012XX3141 | 48075 | TSETAGSTNDNT | 0.42 | −0.67 | −0.7 | −1.06 | ATLVT012XX3187 | 48121 | TSQAAGSTNDNA | −1.14 | −1.02 | −3.59 | −3.86 |
| ATLVT012XX3142 | 48076 | TSETGAASNDNA | −2.62 | 0.77 | −2.94 | −4.64 | ATLVT012XX3188 | 48122 | TSQAAGSTNDNH | 0.38 | 0.87 | −2.64 | −1.22 |
| ATLVT012XX3143 | 48077 | TSETGAASNDNH | −1.97 | 5.32 | −0.66 | −3.82 | ATLVT012XX3189 | 48123 | TSQAAGSTNDNT | 1.65 | 0.89 | −1.79 | −1.77 |
| ATLVT012XX3144 | 48078 | TSETGAASNDNT | −1.05 | −0.76 | 0.34 | −4 | ATLVT012XX3190 | 48124 | TSQAGAASNDNA | −2.09 | −1.83 | −2.92 | −2.82 |
| ATLVT012XX3145 | 48079 | TSETGAATNDNA | −2.64 | −1.09 | −1.55 | −1.65 | ATLVT012XX3191 | 48125 | TSQAGAASNDNH | −2.77 | −2.55 | −1.97 | −2.9 |
| ATLVT012XX3146 | 48080 | TSETGAATNDNH | −1.6 | −1.39 | −2.52 | −0.85 | ATLVT012XX3192 | 48126 | TSQAGAASNDNT | −2.76 | −0.01 | −3.39 | −4.11 |
| ATLVT012XX3147 | 48081 | TSETGAATNDNT | −1.18 | −1.99 | −2.35 | −1.89 | ATLVT012XX3193 | 48127 | TSQAGAATNDNA | −1.71 | 0.45 | −2.84 | −2.6 |
| ATLVT012XX3148 | 48082 | TSETGASSNDNA | −2.48 | −0.4 | −0.84 | −1.92 | ATLVT012XX3194 | 48128 | TSQAGAATNDNH | 2.38 | −0.78 | −1.38 | −3.33 |
| ATLVT012XX3149 | 48083 | TSETGASSNDNH | −0.33 | −2.78 | −0.7 | −2.21 | ATLVT012XX3195 | 48129 | TSQAGAATNDNT | −1.21 | −0.29 | −1.27 | −2.27 |
| ATLVT012XX3150 | 48084 | TSETGASSNDNT | −2.56 | −2.3 | −0.33 | −0.74 | ATLVT012XX3196 | 48130 | TSQAGASSNDNA | 1.25 | −1.06 | −1.25 | −1.75 |
| ATLVT012XX3151 | 48085 | TSETGASTNDNA | −2.22 | −1.69 | 0.36 | −3.12 | ATLVT012XX3197 | 48131 | TSQAGASSNDNH | 1.44 | −1.11 | 0.41 | −2.05 |
| ATLVT012XX3152 | 48086 | TSETGASTNDNH | −0.94 | 2.9 | −0.39 | −1.54 | ATLVT012XX3198 | 48132 | TSQAGASSNDNT | −0.91 | 0.19 | 0.95 | −2.29 |
| ATLVT012XX3153 | 48087 | TSETGASTNDNT | 0.16 | −2.05 | −0.98 | −2.45 | ATLVT012XX3199 | 48133 | TSQAGASTNDNA | −0.15 | 0.62 | 0 | 3.3 |
| ATLVT012XX3154 | 48088 | TSETGGASNDNA | 0.23 | 1.52 | −0.07 | 1.54 | ATLVT012XX3200 | 48134 | TSQAGASTNDNH | −0.72 | −1.53 | −0.28 | −1.64 |
| ATLVT012XX3155 | 48089 | TSETGGASNDNH | 1.09 | −0.08 | 0.65 | 1.44 | ATLVT012XX3201 | 48135 | TSQAGASTNDNT | −0.94 | 0.13 | −0.33 | −0.03 |
| ATLVT012XX3156 | 48090 | TSETGGASNDNT | 1.39 | 1.1 | 0.45 | −1.19 | ATLVT012XX3202 | 48136 | TSQAGGASNDNA | 1.29 | 0.94 | −0.94 | 0.81 |
| ATLVT012XX3157 | 48091 | TSETGGATNDNA | −0.92 | −1.14 | −0.12 | 0.58 | ATLVT012XX3203 | 48137 | TSQAGGASNDNH | 2.63 | 2.07 | −1.94 | 1.68 |
| ATLVT012XX3158 | 48092 | TSETGGATNDNH | 1.78 | 2.46 | 0.25 | 2.94 | ATLVT012XX3204 | 48138 | TSQAGGASNDNT | 2.32 | 0.44 | −0.31 | −0.34 |
| ATLVT012XX3159 | 48093 | TSETGGATNDNT | 0.73 | 0.83 | 0.16 | −0.28 | ATLVT012XX3205 | 48139 | TSQAGGATNDNA | 1.43 | 1.83 | −2.08 | 0.98 |
| ATLVT012XX3160 | 48094 | TSETGGSSNDNA | −1.18 | −0.48 | 0.61 | 0.17 | ATLVT012XX3206 | 48140 | TSQAGGATNDNH | 7.06 | 4.19 | −0.27 | 2.54 |
| ATLVT012XX3161 | 48095 | TSETGGSSNDNH | −1.93 | 3.58 | −0.82 | −2.09 | ATLVT012XX3207 | 48141 | TSQAGGATNDNT | 4.17 | 0.79 | −1.45 | 0.25 |
| ATLVT012XX3162 | 48096 | TSETGGSSNDNT | −1.02 | −0.55 | −0.86 | −2.39 | ATLVT012XX3208 | 48142 | TSQAGGSSNDNA | 0.17 | −1.11 | −0.65 | −0.18 |
| ATLVT012XX3163 | 48097 | TSETGGSTNDNA | −0.56 | 0.58 | −0.56 | −1.05 | ATLVT012XX3209 | 48143 | TSQAGGSSNDNH | −2.01 | −0.59 | 0.39 | 1.48 |
| ATLVT012XX3164 | 48098 | TSETGGSTNDNH | −0.11 | 2.6 | −0.56 | 1.01 | ATLVT012XX3210 | 48144 | TSQAGGSSNDNT | −0.17 | −1.27 | −0.33 | −1.37 |
| ATLVT012XX3165 | 48099 | TSETGGSTNDNT | 0.44 | 1.25 | −0.09 | −0.91 | ATLVT012XX3211 | 48145 | TSQAGGSTNDNA | 1.01 | 0.81 | −1.47 | 0.83 |
| ATLVT012XX3166 | 48100 | TSQAAAASNDNA | 0.76 | 3.59 | 0.31 | −0.16 | ATLVT012XX3212 | 48146 | TSQAGGSTNDNH | 0.86 | 0.78 | −0.07 | 1.67 |
| ATLVT012XX3167 | 48101 | TSQAAAASNDNH | 3.43 | 1.62 | 0.19 | −1.87 | ATLVT012XX3213 | 48147 | TSQAGGSTNDNT | 0.6 | 1.27 | 1.62 | −0.58 |
| ATLVT012XX3168 | 48102 | TSQAAAASNDNT | 2.44 | 2.15 | −0.74 | −0.8 | ATLVT012XX3214 | 48148 | TSQSAAASNDNA | −0.5 | −0.12 | 0.05 | −1.3 |
| ATLVT012XX3169 | 48103 | TSQAAAATNDNA | 0.12 | −0.16 | 0.25 | 0.11 | ATLVT012XX3215 | 48149 | TSQSAAASNDNH | −0.21 | 2.85 | −0.59 | −0.89 |
| ATLVT012XX3170 | 48104 | TSQAAAATNDNH | 3.78 | −0.2 | −0.61 | 0.85 | ATLVT012XX3216 | 48150 | TSQSAAASNDNT | −1.73 | 3.76 | −1.04 | −1.54 |
| ATLVT012XX3171 | 48105 | TSQAAAATNDNT | 1.34 | 2.25 | −0.11 | 0.13 | ATLVT012XX3217 | 48151 | TSQSAAATNDNA | −1.21 | −0.12 | −0.83 | −1.33 |
| ATLVT012XX3172 | 48106 | TSQAAASSNDNA | −1.38 | −0.22 | −0.57 | −1.53 | ATLVT012XX3218 | 48152 | TSQSAAATNDNH | 0.16 | 1.45 | −0.34 | −1.87 |
| ATLVT012XX3173 | 48107 | TSQAAASSNDNH | 1.82 | −2.14 | −0.03 | −3.45 | ATLVT012XX3219 | 48153 | TSQSAAATNDNT | −1.93 | 0.86 | −2.11 | −1.27 |
| ATLVT012XX3174 | 48108 | TSQAAASSNDNT | 0.71 | −2.21 | −0.67 | −0.89 | ATLVT012XX3220 | 48154 | TSQSAASSNDNA | −0.85 | −1.28 | −1.08 | −0.2 |
| ATLVT012XX3221 | 48155 | TSQSAASSNDNH | −1.72 | −2.21 | 0.41 | −2.21 | ATLVT012XX3267 | 48201 | TSQTAAATNDNT | 0.81 | −1.15 | 0.4 | 1.33 |
| ATLVT01XXX3222 | 48156 | TSQSAASSNDNT | 1.18 | −2.13 | 0.8 | −1.66 | ATLVT012XX3268 | 48202 | TSQTAASSNDNA | −1.55 | 0.25 | −0.46 | 0.73 |
| ATLVT012XX3223 | 48157 | TSQSAASTNDNA | −0.05 | 0.4 | −1.19 | 0.57 | ATLVT012XX3269 | 48203 | TSQTAASSNDNH | −1.55 | 0.46 | 0 | −1.89 |
| ATLVT012XX3224 | 48158 | TSQSAASTNDNH | −2.45 | 1.03 | 0.59 | 1.91 | ATLVT012XX3270 | 48204 | TSQTAASSNDNT | −1.85 | 0.76 | −1.76 | −0.22 |
| ATLVT012XX3225 | 48159 | TSQSAASTNDNT | −1.26 | 0.67 | 0.16 | 1.24 | ATLVT012XX3271 | 48205 | TSQTAASTNDNA | 1.55 | −1.57 | −0.64 | 0.03 |
| ATLVT012XX3226 | 48160 | TSQSAGASNDNA | −1.89 | −0.37 | −0.75 | −1.76 | ATLVT012XX3272 | 48206 | TSQTAASTNDNH | 1.63 | −1.69 | −1.23 | −0.09 |
| ATLVT012XX3227 | 48161 | TSQSAGASNDNH | −0.99 | 1.59 | −1.6 | −0.66 | ATLVT012XX3273 | 48207 | TSQTAASTNDNT | 0.84 | −1.43 | −0.53 | 1.71 |
| ATLVT012XX3228 | 48162 | TSQSAGASNDNT | 1.26 | 0.89 | −0.89 | −1.55 | ATLVT012XX3274 | 48208 | TSQTAGASNDNA | 2.93 | 3.33 | −2.81 | −2.04 |
| ATLVT012XX3229 | 48163 | TSQSAGATNDNA | 3.34 | −0.96 | 0.12 | 0.25 | ATLVT012XX3275 | 48209 | TSQTAGASNDNH | 1.61 | 0.63 | −0.31 | −1.42 |
| ATLVT01ZXX3230 | 48164 | TSQSAGATNDNH | 3.83 | −0.32 | 0.07 | 2.23 | ATLVT012XX3276 | 48210 | TSQTAGASNDNT | 2.36 | 2.62 | −2.15 | −1.59 |
| ATLVT012XX3231 | 48165 | TSQSAGATNDNT | 2.06 | 3.42 | −2.05 | 1.05 | ATLVT012XX3277 | 48211 | TSQTAGATNDNA | −1.53 | −0.77 | −0.31 | −0.67 |
| ATLVT012XX3232 | 48166 | TSQSAGSSNDNA | −1.57 | −1.3 | −1.21 | 1.28 | ATLVT012XX3278 | 48212 | TSQTAGATNDNH | 2.66 | 2.7 | 0.28 | 1.11 |
| ATLVT012XX3233 | 48167 | TSQSAGSSNDNH | −1.04 | 1.4 | 0.1 | 0.52 | ATLVT012XX3279 | 48213 | TSQTAGATNDNT | −0.36 | −0.5 | −1.39 | −2.04 |
| ATLVT012XX3234 | 48168 | TSQSAGSSNDNT | −0.17 | −0.97 | −0.57 | −1.87 | ATLVT012XX3280 | 48214 | TSQTAGSSNDNA | −0.55 | −0.83 | −1.21 | −2.31 |
| ATLVT012XX3235 | 48169 | TSQSAGSTNDNA | 0.7 | −0.69 | 0.46 | −1.28 | ATLVT012XX3281 | 48215 | TSQTAGSSNDNH | 0 | −1.47 | −0.89 | 1.97 |
| ATLVT012XX3236 | 48170 | TSQSAGSTNDNH | 0.81 | 0.32 | −0.77 | −1.53 | ATLVT012XX3282 | 48216 | TSQTAGSSNDNT | 0.47 | 0.28 | −1.29 | −0.95 |
| ATLVT012XX3237 | 48171 | TSQSAGSTNDNT | −0.25 | −0.81 | 0.09 | −0.6 | ATLVT012XX3283 | 48217 | TSQTAGSTNDNA | 0.01 | −1.48 | −0.68 | 0.49 |
| ATLVT012XX3238 | 48172 | TSQSGAASNDNA | −0.49 | −1.41 | −0.62 | −0.22 | ATLVT012XX3284 | 48218 | TSQTAGSTNDNH | −0.07 | −1.56 | −0.8 | 0.7 |
| ATLVT012XX3239 | 48173 | TSQSGAASNDNH | −1.52 | −1.12 | −0.33 | −0.86 | ATLVT012XX3285 | 48219 | TSQTAGSTNDNT | 1.18 | −1.81 | −0.07 | −0.2 |
| ATLVT012XX3240 | 48174 | TSQSGAASNDNT | −1.12 | 1.1 | −0.08 | −1.98 | ATLVT012XX3286 | 48220 | TSQTGAASNDNA | −0.88 | −0.72 | −2.28 | −1.63 |
| ATLVT012XX3241 | 48175 | TSQSGAATNDNA | −0.81 | −0.14 | −0.17 | −1.91 | ATLVT012XX3287 | 48221 | TSQTGAASNDNH | −0.87 | 0.64 | −1.74 | −0.53 |
| ATLVT012XX3242 | 48176 | TSQSGAATNDNH | −0.72 | −0.9 | −0.71 | 0.57 | ATLVT012XX3288 | 48222 | TSQTGAASNDNT | 0.57 | 1 | −2.3 | −2.03 |
| ATLVT01XXX3243 | 48177 | TSQSGAATNDNT | −0.09 | −0.74 | −1.45 | 0.18 | ATLVT012XX3289 | 48223 | TSQTGAATNDNA | −0.82 | −0.3 | −1.47 | −1.57 |
| ATLVT012XX3244 | 48178 | TSQSGASSNDNA | 0.48 | −1.84 | −2.11 | 0.08 | ATLVT012XX3290 | 48224 | TSQTGAATNDNH | 0.19 | −0.59 | −1.2 | −2.21 |
| ATLVT012XX3245 | 48179 | TSQSGASSNDNH | −0.74 | −2.01 | −0.9 | −0.63 | ATLVT012XX3291 | 48225 | TSQTGAATNDNT | 0.22 | −0.88 | −1.88 | −1.53 |
| ATLVT012XX3246 | 48180 | TSQSGASSNDNT | 1.54 | −2.57 | −1.86 | 0.11 | ATLVT012XX3292 | 48226 | TSQTGASSNDNA | −1.42 | −1.65 | −1.25 | 0.05 |
| ATLVT012XX3247 | 48181 | TSQSGASTNDNA | −1.66 | −0.73 | 1.01 | −1.52 | ATLVT012XX3293 | 48227 | TSQTGASSNDNH | −1.16 | −1.25 | −0.64 | −1.5 |
| ATLVT012XX3248 | 48182 | TSQSGASTNDNH | −1.02 | −1.89 | 1.12 | −0.04 | ATLVT012XX3294 | 48228 | TSQTGASSNDNT | −1.19 | −2.23 | −1.71 | −1.18 |
| ATLVT012XX3249 | 48183 | TSQSGASTNDNT | 1.56 | 0.21 | −0.68 | −1.04 | ATLVT012XX3295 | 48229 | TSQTGASTNDNA | −1.78 | 0.33 | 1.59 | −1.05 |
| ATLVT012XX3250 | 48184 | TSQSGGASNDNA | 2.19 | 3.05 | −0.14 | −1.13 | ATLVT012XX3296 | 48230 | TSQTGASTNDNH | −1.88 | 1.4 | −0.51 | 0.43 |
| ATLVT012XX3251 | 48185 | TSQSGGASNDNH | 3.45 | 5.8 | 1.09 | 2.97 | ATLVT012XX3297 | 48231 | TSQTGASTNDNT | −1.06 | 2.52 | −0.01 | −1.3 |
| ATLVT012XX3252 | 48186 | TSQSGGASNDNT | 2.23 | 2.09 | −0.27 | 0.04 | ATLVT012XX3298 | 48232 | TSQTGGASNDNA | 2.44 | 4.93 | 0.7 | 2.86 |
| ATLVT012XX3253 | 48187 | TSQSGGATNDNA | 2.88 | 4.06 | 0.67 | 1.19 | ATLVT012XX3299 | 48233 | TSQTGGASNDNH | 2.1 | 2.18 | −0.45 | 2.14 |
| ATLVT012XX3254 | 48188 | TSQSGGATNDNH | 7 | 7.59 | 0.64 | 4.47 | ATLVT012XX3300 | 48234 | TSQTGGASNDNT | 2.3 | 2.42 | −0.49 | 0.78 |
| ATLVT012XX3255 | 48189 | TSQSGGATNDNT | 2.66 | 2.47 | 0.81 | 0.6 | ATLVT012XX3301 | 48235 | TSQTGGATNDNA | 4.03 | 2.06 | −0.34 | 1.53 |
| ATLVT012XX3256 | 48190 | TSQSGGSSNDNA | 0.42 | 0.91 | 0.89 | 0.15 | ATLVT012XX3302 | 48236 | TSQTGGATNDNH | 7.75 | 8.54 | 0.7 | 4.36 |
| ATLVT012XX3257 | 48191 | TSQSGGSSNDNH | 1.18 | 0.55 | 0.07 | 1.33 | ATLVT012XX3303 | 48237 | TSQTGGATNDNT | 4.25 | 2.93 | 0.18 | 0.67 |
| ATLVT012XX3258 | 48192 | TSQSGGSSNDNT | 1.08 | 0.36 | 1.46 | −1.27 | ATLVT012XX3304 | 48238 | TSQTGGSSNDNA | 0.13 | −0.39 | −1.21 | −1.67 |
| ATLVT01XXX3259 | 48193 | TSQSGGSTNDNA | 1.58 | 0.97 | 0.19 | 0.76 | ATLVT012XX3305 | 48239 | TSQTGGSSNDNH | 1.43 | −0.18 | −1.33 | −0.23 |
| ATLVT012XX3260 | 48194 | TSQSGGSTNDNH | 1.06 | 3.08 | −0.97 | 1.36 | ATLVT012XX3306 | 48240 | TSQTGGSSNDNT | 0.94 | 0.06 | −1.31 | 0.36 |
| ATLVT012XX3261 | 48195 | TSQSGGSTNDNT | 1.36 | 2.18 | −0.52 | 1 | ATLVT012XX3307 | 48241 | TSQTGGSTNDNA | 3.95 | 1.51 | 0.17 | 2.61 |
| ATLVT012XX3262 | 48196 | TSQTAAASNDNA | −1.92 | 0.81 | −0.07 | −1.8 | ATLVT012XX3308 | 48242 | TSQTGGSTNDNH | 2.77 | 2.54 | −0.28 | 3.16 |
| ATLVT012XX3263 | 48197 | TSQTAAASNDNH | −0.7 | −0.42 | −0.49 | −3.56 | ATLVT012XX3309 | 48243 | TSQTGGSTNDNT | 3.46 | 3.02 | −0.3 | −0.26 |
| ATLVT012XX3264 | 48198 | TSQTAAASNDNT | −0.44 | −0.6 | −0.92 | −1.65 | ATLVT012XX3310 | 48244 | TSTAAAASNDNA | 0.73 | −1.12 | −1.32 | 1.5 |
| ATLVT012XX3265 | 48199 | TSQTAAATNDNA | −1.15 | −0.71 | −1.14 | 0.75 | ATLVT012XX3311 | 48245 | TSTAAAASNDNH | 0.74 | −0.89 | −0.61 | −0.55 |
| ATLVT012XX3266 | 48200 | TSQTAAATNDNH | 0.84 | 2.72 | −1.11 | −0.77 | ATLVT012XX3312 | 48246 | TSTAAAASNDNT | 1.92 | −0.22 | −1.53 | 0.12 |
| ATLVT012XX3313 | 48247 | TSTAAAATNDNA | 1.58 | −1.22 | −1.08 | 0.31 | ATLVT012XX3359 | 48293 | TSTSAAASNDNH | 2.11 | −0.44 | −0.71 | −0.64 |
| ATLVT012XX3314 | 48248 | TSTAAAATNDNH | 2.76 | −0.07 | −0.01 | −0.69 | ATLVT012XX3360 | 48294 | TSTSAAASNDNT | 2.1 | 1.62 | −0.72 | −1.29 |
| ATLVT012XX3315 | 48249 | TSTAAAATNDNT | 3.73 | 1.01 | 0.08 | −0.9 | ATLVT012XX3361 | 48295 | TSTSAAATNDNA | 2.3 | −0.19 | −0.95 | −0.14 |
| ATLVT012XX3316 | 48250 | TSTAAASSNDNA | 0.93 | 1.15 | −1.08 | 0.55 | ATLVT012XX3362 | 48296 | TSTSAAATNDNH | 0.85 | 1.98 | −0.81 | −0.59 |
| ATLVT012XX3317 | 48251 | TSTAAASSNDNH | −1.85 | −1.37 | −0.83 | −0.14 | ATLVT012XX3363 | 48297 | TSTSAAATNDNT | 1.57 | −0.12 | −0.63 | −1.96 |
| ATLVT012XX3318 | 48252 | TSTAAASSNDNT | 0.49 | 0.49 | −0.18 | 1.5 | ATLVT012XX3364 | 48298 | TSTSAASSNDNA | −0.14 | −0.36 | −0.65 | −1.61 |
| ATLVT012XX3319 | 48253 | TSTAAASTNDNA | 1.53 | −1.25 | −0.32 | −1.07 | ATLVT012XX3365 | 48299 | TSTSAASSNDNH | −1.84 | −1.22 | 0.72 | −0.12 |
| ATLVT012XX3320 | 48254 | TSTAAASTNDNH | 0.34 | −1.49 | −0.17 | −0.1 | ATLVT012XX3366 | 48300 | TSTSAASSNDNT | −0.5 | 0.24 | −0.92 | −1.5 |
| ATLVT012XX3321 | 48255 | TSTAAASTNDNT | 3.29 | −2.11 | −0.99 | −0.22 | ATLVT012XX3367 | 48301 | TSTSAASTNDNA | −1.5 | 2.73 | 0.08 | −0.21 |
| ATLVT012XX3322 | 48256 | TSTAAGASNDNA | −1.22 | −0.66 | 0.02 | −0.8 | ATLVT012XX3368 | 48302 | TSTSAASTNDNH | −1.79 | 2.84 | −0.83 | −0.83 |
| ATLVT012XX3323 | 48257 | TSTAAGASNDNH | −0.68 | −0.04 | −0.31 | −0.29 | ATLVT012XX3369 | 48303 | TSTSAASTNDNT | −2.11 | 1.99 | −0.4 | −1.13 |
| ATLVT012XX3324 | 48258 | TSTAAGASNDNT | −0.08 | 0.02 | −0.55 | −1.8 | ATLVT012XX3370 | 48304 | TSTSAGASNDNA | −0.55 | 5.11 | −1.15 | 2.38 |
| ATLVT012XX3325 | 48259 | TSTAAGATNDNA | −1.33 | 0.53 | 0 | 1.29 | ATLVT012XX3371 | 48305 | TSTSAGASNDNH | −1.33 | 1.37 | 0.18 | 1.05 |
| ATLVT012XX3326 | 48260 | TSTAAGATNDNH | 1.75 | 2.19 | −0.18 | 3.68 | ATLVT012XX3372 | 48306 | TSTSAGASNDNT | −0.36 | 1.5 | −0.04 | 1.8 |
| ATLVT012XX3327 | 48261 | TSTAAGATNDNT | 0.25 | 1.02 | −0.65 | −0.11 | ATLVT012XX3373 | 48307 | TSTSAGATNDNA | −3.03 | 3.25 | 1.03 | 1.73 |
| ATLVT012XX3328 | 48262 | TSTAAGSSNDNA | 0.24 | −2.33 | −1.07 | 0.61 | ATLVT012XX3374 | 48308 | TSTSAGATNDNH | −0.65 | 4.86 | −0.31 | 3.05 |
| ATLVT012XX3329 | 48263 | TSTAAGSSNDNH | −2.32 | −2.27 | −0.07 | 2.17 | ATLVT012XX3375 | 48309 | TSTSAGATNDNT | −0.81 | 1.24 | −0.03 | 1.07 |
| ATLVT012XX3330 | 48264 | TSTAAGSSNDNT | 1.26 | −2.05 | −1.21 | 0.15 | ATLVT012XX3376 | 48310 | TSTSAGSSNDNA | 2.18 | 0.2 | −2.76 | −1.19 |
| ATLVT012XX3331 | 48265 | TSTAAGSTNDNA | 0.41 | −0.04 | −0.67 | −0.45 | ATLVT012XX3377 | 48311 | TSTSAGSSNDNH | −0.91 | −0.71 | −1.56 | −2.26 |
| ATLVT012XX3332 | 48266 | TSTAAGSTNDNH | −1.01 | −1.6 | −1.11 | −0.26 | ATLVT012XX3378 | 48312 | TSTSAGSSNDNT | 1.69 | 2.14 | −1.55 | −1.59 |
| ATLVT012XX3333 | 48267 | TSTAAGSTNDNT | 0.98 | −0.51 | −2.53 | −0.73 | ATLVT012XX3379 | 48313 | TSTSAGSTNDNA | 0.71 | 2.53 | −0.92 | 0.25 |
| ATLVT012XX3334 | 48268 | TSTAGAASNDNA | 1.06 | −1.08 | −3.26 | −0.47 | ATLVT012XX3380 | 48314 | TSTSAGSTNDNH | −1.82 | 1.11 | 0.91 | 0.99 |
| ATLVT012XX3335 | 48269 | TSTAGAASNDNH | 1.04 | 0.95 | −1.88 | −1.91 | ATLVT012XX3381 | 48315 | TSTSAGSTNDNT | 0.81 | 3.15 | −0.68 | −0.25 |
| ATLVT012XX3336 | 48270 | TSTAGAASNDNT | 0.41 | −1.42 | −2.01 | −0.41 | ATLVT012XX3382 | 48316 | TSTSGAASNDNA | −0.81 | 1.36 | −1.83 | −1.78 |
| ATLVT012XX3337 | 48271 | TSTAGAATNDNA | 1.31 | −0.08 | −1.9 | −0.29 | ATLVT012XX3383 | 48317 | TSTSGAASNDNH | −0.48 | 3.64 | −1.24 | −1.31 |
| ATLVT012XX3338 | 48272 | TSTAGAATNDNH | 0.84 | 0.14 | −0.94 | −0.29 | ATLVT012XX3384 | 48318 | TSTSGAASNDNT | −1.45 | 1.09 | −1.44 | −0.54 |
| ATLVT012XX3339 | 48273 | TSTAGAATNDNT | −0.21 | 1.02 | −1.65 | 0.99 | ATLVT012XX3385 | 48319 | TSTSGAATNDNA | −2.85 | 1.97 | −0.14 | −1.33 |
| ATLVT012XX3340 | 48274 | TSTAGASSNDNA | 0.73 | −1.34 | −1.27 | −1.42 | ATLVT012XX3386 | 48320 | TSTSGAATNDNH | −2.18 | 0.78 | 0.2 | 1.56 |
| ATLVT012XX3341 | 48275 | TSTAGASSNDNH | 1.26 | −1.72 | −0.56 | −0.06 | ATLVT012XX3387 | 48321 | TSTSGAATNDNT | −1.67 | 3.39 | −0.1 | −1.3 |
| ATLVT012XX3342 | 48276 | TSTAGASSNDNT | 2.7 | 0.65 | −2.49 | −0.98 | ATLVT012XX3388 | 48322 | TSTSGASSNDNA | 0.02 | −1.66 | −0.68 | −2.34 |
| ATLVT012XX3343 | 48277 | TSTAGASTNDNA | 1.22 | 1.6 | −0.04 | 0.18 | ATLVT012XX3389 | 48323 | TSTSGASSNDNH | −1.09 | −1.92 | 0.83 | −1.32 |
| ATLVT012XX3344 | 48278 | TSTAGASTNDNH | 0.31 | 0.96 | −1.15 | −1.36 | ATLVT012XX3390 | 48324 | TSTSGASSNDNT | 0.12 | −1.12 | −1.2 | −1.68 |
| ATLVT012XX3345 | 48279 | TSTAGASTNDNT | 2.21 | 1.94 | −1.7 | −0.77 | ATLVT012XX3391 | 48325 | TSTSGASTNDNA | −1.33 | −0.21 | −0.27 | −1.1 |
| ATLVT012XX3346 | 48280 | TSTAGGASNDNA | 3.16 | 4.54 | −0.72 | 2.31 | ATLVT012XX3392 | 48326 | TSTSGASTNDNH | −2.71 | 2.06 | 0.58 | 0.82 |
| ATLVT012XX3347 | 48281 | TSTAGGASNDNH | 4.42 | 5.08 | 0.09 | 2.36 | ATLVT012XX3393 | 48327 | TSTSGASTNDNT | 0.08 | 0.17 | 0.95 | −1.57 |
| ATLVT012XX3348 | 48282 | TSTAGGASNDNT | 3.14 | 3.37 | 0.78 | 0.29 | ATLVT012XX3394 | 48328 | TSTSGGASNDNA | 3.36 | 3.99 | −1.09 | 2.32 |
| ATLVT012XX3349 | 48283 | TSTAGGATNDNA | 4.91 | 4.51 | 0.94 | 3.7 | ATLVT012XX3395 | 48329 | TSTSGGASNDNH | 4.71 | 5.12 | 0.46 | 2.41 |
| ATLVT012XX3350 | 48284 | TSTAGGATNDNH | 6.94 | 9.87 | −0.46 | 3.79 | ATLVT012XX3396 | 48330 | TSTSGGASNDNT | 4.05 | 4.39 | 0.69 | 1.27 |
| ATLVT012XX3351 | 48285 | TSTAGGATNDNT | 4.14 | 5.21 | −0.81 | 0.98 | ATLVT012XX3397 | 48331 | TSTSGGATNDNA | 3.23 | 4.7 | 0.26 | 2.39 |
| ATLVT012XX3352 | 48286 | TSTAGGSSNDNA | 2.05 | 2.75 | 0.36 | 2.44 | ATLVT012XX3398 | 48332 | TSTSGGATNDNH | 6.61 | 8.41 | 2.1 | 4.5 |
| ATLVT012XX3353 | 48287 | TSTAGGSSNDNH | 1.84 | 0.23 | −1 | −0.36 | ATLVT012XX3399 | 48333 | TSTSGGATNDNT | 3.61 | 4 | 0.28 | 0.85 |
| ATLVT012XX3354 | 48288 | TSTAGGSSNDNT | 4.17 | 2.37 | −0.4 | 1.29 | ATLVT012XX3400 | 48334 | TSTSGGSSNDNA | 3.08 | 2.74 | −0.54 | 1.86 |
| ATLVT012XX3355 | 48289 | TSTAGGSTNDNA | 4.52 | 5.06 | −0.43 | 2.2 | ATLVT012XX3401 | 48335 | TSTSGGSSNDNH | 0.71 | 1.03 | −0.38 | 0.45 |
| ATLVT012XX3356 | 48290 | TSTAGGSTNDNH | 5.32 | 3.15 | −0.37 | 3.49 | ATLVT012XX3402 | 48336 | TSTSGGSSNDNT | 2.92 | 3.19 | 0.36 | 0.94 |
| ATLVT012XX3357 | 48291 | TSTAGGSTNDNT | 3.51 | 5.13 | −0.28 | −0.48 | ATLVT012XX3403 | 48337 | TSTSGGSTNDNA | 3.58 | 5.96 | 1.23 | 2.33 |
| ATLVT012XX3358 | 48292 | TSTSAAASNDNA | 0.03 | 1.38 | −2.25 | 0.49 | ATLVT012XX3404 | 48338 | TSTSGGSTNDNH | 4.99 | 3.15 | 1.04 | 1.01 |
| ATLVT012XX3405 | 48339 | TSTSGGSTNDNT | 4.3 | 3.99 | 0.43 | −0.11 | ATLVT012XX3451 | 48385 | TSTTGGSTNDNA | 1.72 | 5.01 | 0.55 | 0.39 |
| ATLVT012XX3406 | 48340 | TSTTAAASNDNA | −0.03 | −0.28 | −1.02 | −2.32 | ATLVT012XX3452 | 48386 | TSTTGGSTNDNH | 1.28 | 2.64 | 1.49 | 2.31 |
| ATLVT012XX3407 | 48341 | TSTTAAASNDNH | 0.79 | 3.39 | −1.13 | −2.28 | ATLVT012XX3453 | 48387 | TSTTGGSTNDNT | 4 | 2.02 | −0.68 | −0.13 |
| ATLVT012XX3408 | 48342 | TSTTAAASNDNT | 0.64 | −0.88 | −1.18 | −1.44 | ATLVT012XXAAV9 | 48388 | NSTSGGSSNDNA | 10.85 | 13.11 | 1.65 | 3.83 |
| ATLVT012XX3409 | 48343 | TSTTAAATNDNA | 0.48 | 0 | 0.1 | −2.59 | ATLVT012XXAAV9 | 48389 | NSTSGASSNDNA | 3.35 | 3.29 | 1.18 | 1.56 |
| | | | | | | | L10 | | | | | | |
| ATLVT012XX3410 | 48344 | TSTTAAATNDNH | 0.85 | −1.13 | −0.78 | 0.46 | ATLVT012XXMUT | 48390 | NSTSGASTNDNA | 3.84 | 2.84 | 0.6 | 0 |
| ATLVT012XX3411 | 48345 | TSTTAAATNDNT | 2.01 | −0.7 | −0.7 | −2.67 | | | | | | | |
| ATLVT012XX3412 | 48346 | TSTTAASSNDNA | 0.6 | 0.48 | −0.9 | −1.73 | | | | | | | |
| ATLVT012XX3413 | 48347 | TSTTAASSNDNH | −0.63 | 2.81 | −1.27 | −1.16 | | | | | | | |
| ATLVT012XX3414 | 48348 | TSTTAASSNDNT | −0.69 | 0.38 | −1.34 | −1.46 | | | | | | | |

| | | | | | | |
|---|---|---|---|---|---|---|
| ATLVT012XX3415 | 48349 | TSTTAASTNDNA | −1.53 | 2.58 | 0.71 | 1.68 |
| ATLVT012XX3416 | 48350 | TSTTAASTNDNH | −1.84 | 0.35 | 1.57 | −1.8 |
| ATLVT012XX3417 | 48351 | TSTTAASTNDNT | −0.62 | 2.08 | −1.29 | 0.67 |
| ATLVT012XX3418 | 48352 | TSTTAGASNDNA | −1.07 | 0.27 | −1.74 | 1.52 |
| ATLVT012XX3419 | 48353 | TSTTAGASNDNH | 1.59 | 1.41 | −2.01 | 2.34 |
| ATLVT012XX3420 | 48354 | TSTTAGASNDNT | −0.03 | −0.15 | −1.07 | 0.24 |
| ATLVT012XX3421 | 48355 | TSTTAGATNDNA | 0.07 | 2.43 | −2.02 | 0.08 |
| ATLVT012XX3422 | 48356 | TSTTAGATNDNH | 0.84 | 3.02 | 0.75 | 3.26 |
| ATLVT012XX3423 | 48357 | TSTTAGATNDNT | 0.4 | −0.22 | 0.22 | −0.02 |
| ATLVT012XX3424 | 48358 | TSTTAGSSNDNA | 0.44 | 0.53 | −1.42 | 0.07 |
| ATLVT012XX3425 | 48359 | TSTTAGSSNDNH | −1.39 | −0.02 | −0.93 | −1.77 |
| ATLVT012XX3426 | 48360 | TSTTAGSSNDNT | 2.06 | −0.6 | −2.02 | −0.81 |
| ATLVT012XX3427 | 48361 | TSTTAGSTNDNA | 1 | 0.15 | 0.74 | 0.37 |
| ATLVT012XX3428 | 48362 | TSTTAGSTNDNH | 0.53 | 2.24 | −0.46 | 0.53 |
| ATLVT012XX3429 | 48363 | TSTTAGSTNDNT | 4.42 | 1.9 | −0.79 | 1.15 |
| ATLVT012XX3430 | 48364 | TSTTGAASNDNA | −0.07 | −1.67 | −2.66 | −2.07 |
| ATLVT012XX3431 | 48365 | TSTTGAASNDNH | −1.39 | 0.27 | −1.78 | −2.13 |
| ATLVT012XX3432 | 48366 | TSTTGAASNDNT | −0.79 | −0.15 | −2.77 | −1.77 |
| ATLVT012XX3433 | 48367 | TSTTGAATNDNA | 0.53 | −0.29 | −0.18 | −1.87 |
| ATLVT012XX3434 | 48368 | TSTTGAATNDNH | 1.24 | 0.54 | −1.39 | −0.32 |
| ATLVT012XX3435 | 48369 | TSTTGAATNDNT | 3.07 | 1.76 | −0.39 | −1.55 |
| ATLVT012XX3436 | 48370 | TSTTGASSNDNA | −1.34 | −1.52 | −2.06 | −1.36 |
| ATLVT012XX3437 | 48371 | TSTTGASSNDNH | −0.18 | .1.29 | −1.45 | 1.58 |
| ATLVT012XX3438 | 48372 | TSTTGASSNDNT | 1.62 | 0.99 | −0.37 | −0.64 |
| ATLVT012XX3439 | 48373 | TSTTGASTNDNA | 1.91 | −0.93 | 0.91 | −0.6 |
| ATLVT012XX3440 | 48374 | TSTTGASTNDNH | 0.59 | 0.3 | −0.73 | −1.36 |
| ATLVT012XX3441 | 48375 | TSTTGASTNDNT | 2.7 | 1.7 | −0.24 | −1.65 |
| ATLVT012XX3442 | 48376 | TSTTGGASNDNA | 1.69 | 1.28 | −0.34 | 0.96 |
| ATLVT012XX3443 | 48377 | TSTTGGASNDNH | 3.73 | 3.97 | 0.17 | 2.42 |
| ATLVT012XX3444 | 48378 | TSTTGGASNDNT | 1.71 | 2.11 | −0.39 | 1.28 |
| ATLVT012XX3445 | 48379 | TSTTGGATNDNA | 1.81 | 2.32 | 0.95 | 1.68 |
| ATLVT012XX3446 | 48380 | TSTTGGATNDNH | 5.6 | 5.73 | 1.1 | 4.5 |
| ATLVT012XX3447 | 48381 | TSTTGGATNDNT | 2.65 | 3.46 | −0.21 | 2.45 |
| ATLVT012XX3448 | 48382 | TSTTGGSSNDNA | 3.4 | 2.76 | 0.14 | 0.03 |
| ATLVT012XX3449 | 48383 | TSTTGGSSNDNH | 3.08 | 0.08 | 0.37 | 2.44 |
| ATLVT012XX3450 | 48384 | TSTTGGSSNDNT | 4.31 | 1.1 | −0.82 | 1.08 |

Provided below is Table 29. Abbreviations for Table 29 are as follows: Bicep—Biceps femoris, Quad—Quadriceps, Diap.—Diaphragm, Atria—Heart—Atria, Vent.—Heart—Ventricle Wall, and Liver—Liver lobe. Table 29 shows individual tissue enrichment scores for RNA for each AAV variant. Data is provided for tissues including Bicep, Quad, Diaph. Atria, Vent., and Liver.

Lengthy table referenced here
US20260062450A1-20260305-T00002
Please refer to the end of the specification for access instructions.

Provided below is Table 30. Abbreviations for Table 30 are as follows: Diaph.—Diapgrahm. Table 30 shows individual tissues enrichment scores for DNA for each AAV. Data is provided for tissues including Diaph., Heart, and Liver.

| | | SEQ ID | | | | |
|---|---|---|---|---|---|---|
| Rank | variant_id | NO: | Sequence | Diaph. | Heart | Liver |
| 1 | ATLVT012XXAAV9 | 48388 | NSTSGGSSNDNA | 11.26 | 21.08 | 19.61 |
| 2 | ATLVT012XX1099 | 46033 | NSTSGGSTNDNA | 8.69 | 11.7 | 18.88 |
| 3 | ATLVT012XX1094 | 46028 | NSTSGGAINDNA | 9.77 | 11.36 | 16.69 |
| 4 | ATLVT012XX1100 | 46034 | NSTSGGSINDNH | 8.96 | 12.75 | 16.17 |
| 5 | ATLVT012XX1097 | 46031 | NSTSGGSSNDNH | 8.93 | 11.1 | 17.04 |
| 6 | ATLVT012XX1098 | 46032 | NSTSGGSSNDNT | 7.1 | 12.65 | 17.96 |
| 7 | ATLVT012XX1054 | 45988 | NSTAGGSTNDNA | 9.44 | 8.55 | 18.55 |
| 8 | ATLVT012XX1718 | 46652 | SGTTGGAINDNH | 9.02 | 11.49 | 14.24 |
| 9 | ATLVT012XX1147 | 46081 | NSTTGGSINDNA | 7.39 | 10.36 | 18.33 |
| 10 | ATLVT012XX1101 | 46035 | NSTSGGSTNDNT | 7.29 | 10.97 | 17.33 |
| 11 | ATLVT012XX1144 | 46078 | NSTTGGSSNDNA | 6.52 | 12.04 | 17.21 |
| 12 | ATLVT012XX1091 | 46025 | NSTSGGASNDNA | 6.47 | 11.28 | 17.71 |
| 13 | ATLVT012XX1051 | 45985 | NSTAGGSSNDNA | 6.16 | 11.23 | 18.3 |
| 14 | ATLVT012XX2246 | 47180 | SSTSGGATNDNH | 7.85 | 10.16 | 16.7 |
| 15 | ATLVT012XX1093 | 46027 | NSTSGGASNDNT | 6.54 | 9.78 | 18.77 |
| 16 | ATLVT012XX1149 | 46083 | NSTTGGSTNDNT | 6.24 | 10.29 | 18.46 |
| 17 | ATLVT012XX523 | 45457 | NGTSGGSSNDNA | 7.82 | 10.99 | 14.84 |
| 18 | ATLVT012XX1053 | 45987 | NSTAGGSSNDNT | 6.11 | 10.03 | 18.82 |
| 19 | ATLVT012XX2243 | 47177 | SSTSGGASNDNH | 7.76 | 10.44 | 15.33 |
| 20 | ATLVT012XX1096 | 46030 | NSTSGGATNDNT | 5.83 | 10.7 | 18.3 |
| 21 | ATLVT012XX1670 | 46604 | SGTSGGATNDNH | 7.4 | 10.6 | 14.42 |
| 22 | ATLVT012XX1484 | 46418 | SGQAGGSINDNH | 6.56 | 11.83 | 13.35 |
| 23 | ATLVT012XX2245 | 47179 | SSTSGGATNDNA | 7.25 | 11.27 | 12.73 |
| 24 | ATLVT012XX1142 | 46076 | NSTTGGAINDNH | 8.32 | 9.91 | 12.26 |
| 25 | ATLVT012XX1667 | 46601 | SGTSGGASNDNH | 8.08 | 9.25 | 13.33 |
| 26 | ATLVT012XX1056 | 45990 | NSTAGGSINDNT | 6.42 | 9 | 16.44 |
| 27 | ATLVT012XX1095 | 46029 | NSTSGGAINDNH | 7.35 | 9.55 | 13.74 |
| 28 | ATLVT012XX1146 | 46080 | NSTTGGSSNDNT | 4.38 | 9.72 | 18.52 |
| 29 | ATLVT012XX2294 | 47228 | SSTTGGATNDNH | 8.07 | 8.33 | 13.8 |
| 30 | ATLVT012XX1055 | 45989 | NSTAGGSTNDNH | 7.57 | 9.8 | 12.76 |
| 31 | ATLVT012XX1047 | 45981 | NSTAGGASNDNT | 7.32 | 6.43 | 17.28 |
| 32 | ATLVT012XX1045 | 45979 | NSTAGGASNDNA | 5.47 | 10.24 | 14.81 |
| 33 | ATLVT012XX1141 | 46075 | NSTIGGATNDNA | 8.09 | 9.81 | 10.79 |
| 34 | ATLVT012XX1092 | 46026 | NSTSGGASNDNH | 7.04 | 7.89 | 14.34 |
| 35 | ATLVT012XX528 | 45462 | NGTSGGSINDNT | 6.23 | 7.79 | 15.85 |
| 36 | ATLVT012XX1675 | 46609 | SGTSGGSINDNA | 8.34 | 7.56 | 12.42 |
| 37 | ATLVT012XX1148 | 46082 | NSTTGGSTNDNH | 7.59 | 7.87 | 13.09 |
| 38 | ATLVT012XX1676 | 46610 | SGTSGGSINDNH | 7.35 | 8.76 | 12.31 |
| 39 | ATLVT012XX571 | 45505 | NGTTGGSSNDNA | 7.68 | 8.14 | 12.16 |
| 40 | ATLVT012XX1052 | 45986 | NSTAGGSSNDNH | 6.03 | 8.92 | 13.8 |
| 41 | ATLVT012XX1140 | 46074 | NSTTGGASNDNT | 4.62 | 9.31 | 15.6 |
| 42 | ATLVT012XX1529 | 46463 | SGQSGGSSNDNH | 10.13 | 6.32 | 9.6 |
| 43 | ATLVT012XX1666 | 46600 | SGTSGGASNDNA | 8.04 | 8.48 | 10.31 |
| 44 | ATLVT012XX1143 | 46077 | NSTTGGAINDNT | 6.1 | 7.87 | 14.37 |
| 45 | ATLVT012XX1580 | 46514 | SGQTGGSINDNH | 7.4 | 9.22 | 10.25 |
| 46 | ATLVT012XX1574 | 46508 | SGQTGGATNDNH | 6.97 | 7.32 | 13.42 |
| 47 | ATLVT012XX2054 | 46988 | SSQAGGATNDNH | 5.35 | 9.86 | 12.93 |
| 48 | ATLVT012XX2251 | 47185 | SSTSGGSTNDNA | 5.82 | 10.22 | 11.43 |
| 49 | ATLVT012XX2248 | 47182 | SSTSGGSSNDNA | 7.39 | 9.43 | 9.71 |
| 50 | ATLVT012XX1532 | 46466 | SGQSGGSINDNH | 5.25 | 9.97 | 12.2 |
| 51 | ATLVT012XX2242 | 47176 | SSTSGGASNDNA | 5.92 | 8.57 | 12.68 |
| 52 | ATLVT012XX526 | 45460 | NGTSGGSINDNA | 6.74 | 7.48 | 12.57 |
| 53 | ATLVT012XX1244 | 46178 | SGASGGSINDNH | 7.97 | 9.41 | 7.95 |
| 54 | ATLVT012XX1579 | 46513 | SGQTGGSINDNA | 5.34 | 10.71 | 10.65 |
| 55 | ATLVT012XX1720 | 46654 | SGTTGGSSNDNA | 6.16 | 9.07 | 10.95 |
| 56 | ATLVT012XX2102 | 47036 | SSQSGGATNDNH | 5.66 | 9.05 | 11.81 |
| 57 | ATLVT012XX525 | 45459 | NGTSGGSSNDNT | 5.23 | 6.43 | 15.89 |
| 58 | ATLVT012XX2108 | 47042 | SSQSGGSINDNH | 8.25 | 9.5 | 6.64 |
| 59 | ATLVT012XX1719 | 46653 | SGTTGGATNDNT | 6.96 | 8.91 | 9.59 |
| 60 | ATLVT012XX2252 | 47186 | SSTSGGSINDNH | 7.08 | 9.42 | 8.72 |
| 61 | ATLVT012XX1715 | 46649 | SGTTGGASNDNH | 5.11 | 9.59 | 11.78 |
| 62 | ATLVT012XX1145 | 46079 | NSTTGGSSNDNH | 4.26 | 9.95 | 12.54 |
| 63 | ATLVT012XX1526 | 46460 | SGQSGGATNDNH | 5.71 | 7.86 | 12.68 |
| 64 | ATLVT012XX1668 | 46602 | SGTSGGASNDNI | 7.37 | 7.46 | 10.34 |
| 65 | ATLVT012XX527 | 45461 | NGTSGGSINDNH | 6.47 | 8.51 | 10.52 |
| 66 | ATLVT012XX2244 | 47178 | SSTSGGASNDNT | 5.83 | 7.69 | 12.28 |
| 67 | ATLVT012XX1046 | 45980 | NSTAGGASNDNH | 6.06 | 6.08 | 13.61 |
| 68 | ATLVT012XX1049 | 45983 | NSTAGGATNDNH | 6.07 | 6.27 | 13.11 |
| 69 | ATLVT012XX1531 | 46465 | SGQSGGSINDNA | 5.24 | 9.4 | 10.5 |
| 70 | ATLVT012XX3446 | 48380 | TSTTGGATNDNH | 6.59 | 9.29 | 8.31 |
| 71 | ATLVT012XX1671 | 46605 | SGTSGGATNDNT | 4.9 | 9.38 | 10.77 |
| 72 | ATLVT012XX2250 | 47184 | SSTSGGSSNDNT | 7.72 | 6.67 | 9.37 |
| 73 | ATLVT012XX2203 | 47137 | SSTAGGSTNDNA | 5.49 | 9.59 | 9.37 |
| 74 | ATLVT012XX1814 | 46748 | SSASGGATNDNH | 6.43 | 10.77 | 6.23 |
| 75 | ATLVT012XX2291 | 47225 | SSTTGGASNDNH | 5.52 | 11.37 | 6.81 |
| 76 | ATLVT012XX1138 | 46072 | NSTTGGASNDNA | 4.8 | 6.48 | 14.2 |
| 77 | ATLVT012XX1139 | 46073 | NSTTGGASNDNH | 5.25 | 8.78 | 10.45 |
| 78 | ATLVT012XX1050 | 45984 | NSTAGGATNDNT | 5.09 | 6.01 | 14.28 |
| 79 | ATLVT012XX576 | 45510 | NGTTGGSINDNT | 6.45 | 6.36 | 11.49 |
| 80 | ATLVT012XX2249 | 47183 | SSTSGGSSNDNH | 7.34 | 7.88 | 7.98 |
| 81 | ATLVT012XX524 | 45458 | NGTSGGSSNDNH | 6.4 | 6.89 | 10.8 |
| 82 | ATLVT012XX1533 | 46467 | SGQSGGSINDNT | 3.5 | 11.85 | 9.36 |
| 83 | ATLVT012XX1528 | 46462 | SGQSGGSSNDNA | 7.93 | 6.53 | 8.46 |
| 84 | ATLVT012XX1382 | 46316 | SGESGGATNDNH | 8.11 | 6.14 | 8.62 |
| 85 | ATLVT012XX3395 | 48329 | TSTSGGASNDNH | 4.27 | 10.33 | 9.68 |
| 86 | ATLVT012XX1523 | 46457 | SGQSGGASNDNH | 5.7 | 7.33 | 10.93 |
| 87 | ATLVT012XX960 | 45894 | NSQSGGSTNDNT | 8.43 | 5.47 | 8.52 |
| 88 | ATLVT012XX1238 | 46172 | SGASGGATNDNH | 5.37 | 7.89 | 10.65 |
| 89 | ATLVT012XX2300 | 47234 | SSTTGGSINDNH | 7.18 | 8.2 | 7.07 |
| 90 | ATLVT012XX1714 | 46648 | SGTTGGASNDNA | 6.11 | 8.36 | 8.66 |
| 91 | ATLVT012XX521 | 45455 | NGTSGGATNDNH | 7.45 | 5.79 | 9.66 |
| 92 | ATLVT012XX1717 | 46651 | SGTTGGATNDNA | 5.45 | 9.22 | 8.67 |
| 93 | ATLVT012XX1475 | 46409 | SGQAGGASNDNH | 4.94 | 7.89 | 11.24 |
| 94 | ATLVT012XX574 | 45508 | NGTTGGSTNDNA | 6.54 | 7.27 | 9.29 |
| 95 | ATLVT012XX1723 | 46657 | SGTTGGSINDNA | 6.19 | 7.07 | 9.96 |
| 96 | ATLVT012XX1724 | 46658 | SGTTGGSTNDNH | 5.42 | 8.09 | 9.92 |
| 97 | ATLVT012XX1722 | 46656 | SGTTGGSSNDNT | 5.89 | 7.3 | 10.09 |
| 98 | ATLVT012XX1672 | 46606 | SGTSGGSSNDNA | 5.82 | 6.96 | 10.63 |
| 99 | ATLVT012XXMUT1 | 48390 | NSTSGASTNDNA | 3.73 | 10.26 | 9.82 |
| 100 | ATLVT012XX517 | 45451 | NGTSGGASNDNA | 5.54 | 8.57 | 8.74 |
| 101 | ATLVT012XX575 | 45509 | NGTTGGSTNDNH | 6.44 | 6.48 | 9.76 |
| 102 | ATLVT012XX3400 | 48334 | TSTSGGSSNDNA | 3.09 | 11.22 | 9.38 |
| 103 | ATLVT012XX2150 | 47084 | SSQTGGATNDNH | 4.32 | 6.83 | 12.91 |
| 104 | ATLVT012XX519 | 45453 | NGTSGGASNDNT | 6.54 | 5.74 | 10.47 |
| 105 | ATLVT012XX1485 | 46419 | SGQAGGSINDNT | 6.45 | 6.19 | 10.03 |
| 106 | ATLVT012XX1483 | 46417 | SGQAGGSTNDNA | 6.14 | 6.75 | 9.82 |
| 107 | ATLVT012XX518 | 45452 | NGTSGGASNDNH | 8.18 | 4.75 | 8.86 |
| 108 | ATLVT012XX1622 | 46556 | SGTAGGATNDNH | 3.96 | 8 | 11.85 |
| 109 | ATLVT012XX1478 | 46412 | SGQAGGATNDNH | 3.81 | 9.14 | 10.57 |
| 110 | ATLVT012XX3398 | 48332 | ISTSGGATNDNH | 4.01 | 9.02 | 10.32 |
| 111 | ATLVT012XX1669 | 46603 | SGTSGGATNDNA | 6.42 | 6.39 | 9.37 |
| 112 | ATLVT012XX520 | 45454 | NGTSGGATNDNA | 7.24 | 6.05 | 8.2 |
| 113 | ATLVT012XX2253 | 47187 | SSTSGGSINDNT | 5.41 | 7.59 | 9.25 |
| 114 | ATLVT012XX2299 | 47233 | SSTIGGSTNDNA | 7.26 | 5.95 | 8.2 |
| 115 | ATLVT012XX2870 | 47804 | TGTTGGATNDNH | 5.09 | 6.66 | 10.78 |
| 116 | ATLVT012XX2099 | 47033 | SSQSGGASNDNH | 6.03 | 6.58 | 9.21 |
| 117 | ATLVT012XX1628 | 46562 | SGTAGGSINDNH | 3.87 | 9.58 | 8.95 |
| 118 | ATLVT012XX522 | 45456 | NGTSGGATNDNT | 4.75 | 6.91 | 10.8 |
| 119 | ATLVT012XX3403 | 48337 | TSTSGGSTNDNA | 4.34 | 8.42 | 9.53 |
| 120 | ATLVT012XX1048 | 45982 | NSTAGGATNDNA | 4.45 | 6.29 | 11.92 |
| 121 | ATLVT012XX1530 | 46464 | SGQSGGSSNDNT | 6.93 | 6.32 | 7.59 |
| 122 | ATLVT012XX477 | 45411 | NGTAGGSSNDNT | 3.86 | 7.4 | 11.31 |
| 123 | ATLVT012XX1436 | 46370 | SGETGGSINDNH | 7.84 | 6.48 | 5.47 |
| 124 | ATLVT012XX573 | 45507 | NGTTGGSSNDNT | 4.16 | 7.38 | 10.63 |
| 125 | ATLVT012XX1430 | 46364 | SGETGGATNDNH | 4.68 | 7.69 | 9.3 |
| 126 | ATLVT012XX2247 | 47181 | SSTSGGATNDNT | 3.72 | 6.99 | 11.63 |
| 127 | ATLVT012XX1627 | 46561 | SGTAGGSINDNA | 4.84 | 5.43 | 11.7 |
| 128 | ATLVT012XX1725 | 46659 | SGTTGGSINDNT | 3.93 | 7.56 | 10.49 |
| 129 | ATLVT012XX2293 | 47227 | SSTTGGATNDNA | 4.5 | 9.42 | 6.94 |
| 130 | ATLVT012XX3302 | 48236 | TSQTGGAINDNH | 3.59 | 6.48 | 12.1 |
| 131 | ATLVT012XX1721 | 46655 | SGTTGGSSNDNH | 6.18 | 5.93 | 8.38 |
| 132 | ATLVT012XX3404 | 48338 | TSTSGGSINDNH | 5.74 | 7.42 | 7.14 |
| 133 | ATLVT012XX3397 | 48331 | TSTSGGATNDNA | 5.77 | 6.41 | 8.22 |
| 134 | ATLVT012XX1623 | 46557 | SGTAGGAINDNT | 2.9 | 9.91 | 8.42 |
| 135 | ATLVT012XX2774 | 47708 | TGTAGGATNDNH | 7.08 | 2.26 | 11.07 |
| 136 | ATLVT012XX1626 | 46560 | SGTAGGSSNDNT | 2.66 | 9.25 | 9.64 |
| 137 | ATLVT012XX1958 | 46892 | SSESGGATNDNH | 7.1 | 7.32 | 4.5 |
| 138 | ATLVT012XX1527 | 46461 | SGQSGGAINDNT | 3.51 | 8.49 | 9.03 |
| 139 | ATLVT012XX2198 | 47132 | SSTAGGATNDNH | 4.15 | 3.92 | 13.77 |
| 140 | ATLVT012XX1677 | 46611 | SGTSGGSINDNT | 4.49 | 5.49 | 11.15 |
| 141 | ATLVT012XX2290 | 47224 | SSTTGGASNDNA | 5.14 | 8.62 | 5.88 |
| 142 | ATLVT012XX2197 | 47131 | SSTAGGATNDNA | 3.98 | 6.27 | 10.88 |
| 143 | ATLVT012XX3299 | 48233 | TSQTGGASNDNH | 4.31 | 8.29 | 7.7 |
| 144 | ATLVT012XX3394 | 48328 | TSTSGGASNDNA | 2.55 | 9.48 | 9.14 |
| 145 | ATLVT012XX1916 | 46850 | SSEAGGSTNDNH | 8.85 | 5.76 | 3.08 |
| 146 | ATLVT012XX1243 | 46177 | SGASGGSINDNA | 6.82 | 6.59 | 5.49 |
| 147 | ATLVT012XX1674 | 46608 | SGTSGGSSNDNI | 5.84 | 4.7 | 9.59 |
| 148 | ATLVT012XX2061 | 46995 | SSQAGGSINDNT | 4.8 | 6.96 | 8.42 |
| 149 | ATLVT012XX1474 | 46408 | SGQAGGASNDNA | 3.52 | 8.55 | 8.39 |
| 150 | ATLVT012XX959 | 45893 | NSQSGGSINDNH | 8.66 | 5.18 | 3.92 |
| 151 | ATLVT012XX1716 | 46650 | SGTTGGASNDNT | 4.75 | 4.39 | 11.62 |
| 152 | ATLVT012XXAAV9L10 | 48389 | NSTSGASSNDNA | 4.36 | 8.76 | 6.67 |
| 153 | ATLVT012XX1673 | 46607 | SGTSGGSSNDNH | 5.57 | 5.83 | 8.32 |
| 154 | ATLVT012XX2107 | 47041 | SSQSGGSINDNA | 6.01 | 6.96 | 6.09 |
| 155 | ATLVT012XX1571 | 46505 | SGQTGGASNDNH | 3.83 | 6.45 | 10.38 |
| 156 | ATLVT012XX2819 | 47753 | TGTSGGASNDNH | 7.29 | 4.28 | 7.16 |
| 157 | ATLVT012XX2205 | 47139 | SSTAGGSINDNT | 4.93 | 5.74 | 9.34 |
| 158 | ATLVT012XX471 | 45405 | NGTAGGASNDNT | 6.34 | 4.98 | 7.6 |
| 159 | ATLVT012XX2301 | 47235 | SSTTGGSTNDNT | 5.68 | 5.01 | 8.67 |
| 160 | ATLVT012XX1581 | 46515 | SGQTGGSINDNT | 3.22 | 7.96 | 9.04 |
| 161 | ATLVT012XX2201 | 47135 | SSTAGGSSNDNH | 4.61 | 7.83 | 6.73 |
| 162 | ATLVT012XX2295 | 47229 | SSTTGGATNDNT | 3.68 | 7.97 | 8.00 |
| 163 | ATLVT012XX1577 | 46511 | SGQTGGSSNDNH | 5.49 | 6.96 | 6.22 |
| 164 | ATLVT012XX1624 | 46558 | SGTAGGSSNDNA | 2.93 | 6.5 | 11.21 |
| 165 | ATLVT012XX1196 | 46130 | SGAAGGSINDNH | 2.79 | 7.46 | 10.12 |
| 166 | ATLVT012XX1573 | 46507 | SGQTGGATNDNA | 7.22 | 3.74 | 7.2 |
| 167 | ATLVT012XX2296 | 47230 | SSTTGGSSNDNA | 6.39 | 5.03 | 6.97 |
| 168 | ATLVT012XX569 | 45503 | NGTTGGATNDNH | 5.06 | 5.79 | 8.16 |
| 169 | ATLVT012XX1235 | 46169 | SGASGGASNDNH | 6.83 | 6.1 | 4.67 |
| 170 | ATLVT012XX958 | 45892 | NSQSGGSTNDNA | 7.97 | 5.14 | 3.95 |
| 171 | ATLVT012XX2109 | 47043 | SSQSGGSTNDNT | 6.08 | 6.47 | 5.47 |
| 172 | ATLVT012XX567 | 45501 | NGTTGGASNDNT | 3.12 | 7.07 | 9.59 |
| 173 | ATLVT012XX1570 | 46504 | SGQTGGASNDNA | 3.57 | 7.03 | 8.77 |
| 174 | ATLVT012XX2105 | 47039 | SSQSGGSSNDNH | 6.35 | 6.17 | 5.03 |
| 175 | ATLVT012XX2195 | 47129 | SSTAGGASNDNH | 4.6 | 4 | 10.75 |
| 176 | ATLVT012XX480 | 45414 | NGTAGGSINDNT | 4.07 | 4.75 | 10.69 |
| 177 | ATLVT012XX3448 | 48382 | TSTTGGSSNDNA | 6.03 | 5.42 | 6.42 |
| 178 | ATLVT012XX1598 | 46532 | SGTAAGATNDNH | 5.76 | 8.56 | 2.75 |
| 179 | ATLVT012XX3444 | 48378 | TSTTGGASNDNT | 6.54 | 4.36 | 6.7 |
| 180 | ATLVT012XX572 | 45506 | NGTTGGSSNDNH | 4.56 | 5.62 | 8.31 |
| 181 | ATLVT012XX3401 | 48335 | TSTSGGSSNDNH | 5.01 | 5.79 | 7.29 |
| 182 | ATLVT012XX2200 | 47134 | SSTAGGSSNDNA | 3.92 | 5.57 | 9.37 |
| 183 | ATLVT012XX1090 | 46024 | NSTSGASTNDNT | 4.67 | 5.99 | 7.49 |
| 184 | ATLVT012XX2147 | 47081 | SSQTGGASNDNH | 4.03 | 6.06 | 8.45 |
| 185 | ATLVT012XX1077 | 46011 | NSTSAGSSNDNT | 1.63 | 9.84 | 7.69 |
| 186 | ATLVT012XX1629 | 46563 | SGTAGGSINDNT | 3.09 | 7.28 | 8.43 |
| 187 | ATLVT012XX3451 | 48385 | TSTTGGSTNDNA | 4.37 | 6.17 | 7.66 |
| 188 | ATLVT012XX1388 | 46322 | SGESGGSINDNH | 4.65 | 5.66 | 7.75 |
| 189 | ATLVT012XX2156 | 47090 | SSQTGGSTNDNH | 5.57 | 6.34 | 5.22 |
| 190 | ATLVT012XX1819 | 46753 | SSASGGSTNDNA | 5.4 | 7.14 | 4.49 |
| 191 | ATLVT012XX1524 | 46458 | SGQSGGASNDNT | 4.45 | 4.74 | 9.19 |
| 192 | ATLVT012XX1130 | 46064 | NSTTGAATNDNH | 5.66 | 4.61 | 7.25 |
| 193 | ATLVT012XX3308 | 48242 | TSQTGGSTNDNH | 3.3 | 9.43 | 5.04 |
| 194 | ATLVT012XX2822 | 47756 | TGTSGGATNDNH | 4.48 | 6.38 | 6.95 |
| 195 | ATLVT012XX3396 | 48330 | TSTSGGASNDNT | 3.02 | 6.68 | 9.03 |
| 196 | ATLVT012XX570 | 45504 | NGTTGGAINDNT | 3.15 | 8 | 7.12 |
| 197 | ATLVT012XX478 | 45412 | NGTAGGSINDNA | 3.12 | 5.21 | 10.46 |
| 198 | ATLVT012XX1480 | 46414 | SGQAGGSSNDNA | 4.38 | 5.92 | 7.29 |
| 199 | ATLVT012XX1084 | 46018 | NSTSGAATNDNA | 5.48 | 5.24 | 6.28 |
| 200 | ATLVT012XX2060 | 46994 | SSQAGGSINDNH | 5 | 4.44 | 8.01 |
| 201 | ATLVT012XX2726 | 47660 | TGQTGGATNDNH | 4.8 | 2 | 11.47 |
| 202 | ATLVT012XX3402 | 48336 | ISTSGGSSNDNT | 4.19 | 4.96 | 8.51 |
| 203 | ATLVT012XX1706 | 46640 | SGTTGAATNDNH | 8.69 | 2.73 | 3.59 |
| 204 | ATLVT012XX2828 | 47762 | TGTSGGSTNDNH | 4.34 | 7.08 | 5.31 |
| 205 | ATLVT012XX2292 | 47226 | SSTTGGASNDNT | 4.37 | 5.84 | 6.83 |
| 206 | ATLVT012XX1525 | 46459 | SGQSGGATNDNA | 5.15 | 4.08 | 7.65 |
| 207 | ATLVT012XX1125 | 46059 | NSTTAGSTNDNT | 2.44 | 8.41 | 6.71 |
| 208 | ATLVT012XX1190 | 46124 | SGAAGGATNDNH | 1.97 | 5.3 | 11.45 |
| 209 | ATLVT012XX2876 | 47810 | TGTTGGSTNDNH | 3.17 | 8.64 | 5.09 |
| 210 | ATLVT012XX1820 | 46754 | SSASGGSINDNH | 6.95 | 6.21 | 1.74 |
| 211 | ATLVT012XX2204 | 47138 | SSTAGGSINDNH | 3.77 | 6.82 | 6.38 |
| 212 | ATLVT012XX3350 | 48284 | TSTAGGATNDNH | 4.1 | 4.9 | 8.26 |
| 213 | ATLVT012XX2146 | 47080 | SSQTGGASNDNA | 4.22 | 5.46 | 7.32 |
| 214 | ATLVT012XX475 | 45409 | NGTAGGSSNDNA | 3.71 | 4.58 | 9 |
| 215 | ATLVT012XX2827 | 47761 | TGTSGGSINDNA | 5.29 | 4.57 | 6.29 |
| 216 | ATLVT012XX1481 | 46415 | SGQAGGSSNDNH | 4.78 | 5.58 | 5.73 |
| 217 | ATLVT012XX2053 | 46987 | SSQAGGATNDNA | 6.13 | 2.88 | 6.88 |
| 218 | ATLVT012XX3260 | 48194 | TSQSGGSINDNH | 3.13 | 6.43 | 7.45 |
| 219 | ATLVT012XX3254 | 48188 | TSQSGGATNDNH | 4.55 | 3.99 | 8.08 |
| 220 | ATLVT012XX1576 | 46510 | SGQTGGSSNDNA | 4.7 | 4.61 | 6.94 |
| 221 | ATLVT012XX1241 | 46175 | SGASGGSSNDNH | 4.81 | 6.37 | 4.49 |
| 222 | ATLVT012XX1479 | 46413 | SGQAGGAINDNT | 3.63 | 6.1 | 6.86 |
| 223 | ATLVT012XX1085 | 46019 | NSTSGAATNDNH | 3.57 | 7.47 | 5.17 |
| 224 | ATLVT012XX2194 | 47128 | SSTAGGASNDNA | 3.57 | 4.92 | 8.39 |
| 225 | ATLVT012XX1910 | 46844 | SSEAGGATNDNH | 4.6 | 6.39 | 4.61 |
| 226 | ATLVT012XX3452 | 48386 | TSTTGGSINDNH | 4.17 | 4.94 | 7.16 |
| 227 | ATLVT012XX2202 | 47136 | SSTAGGSSNDNT | 2.81 | 4.51 | 10.02 |
| 228 | ATLVT012XX2006 | 46940 | SSETGGATNDNH | 5.6 | 4.18 | 5.64 |
| 229 | ATLVT012XX955 | 45889 | NSQSGGSSNDNA | 5.79 | 6.21 | 2.66 |
| 230 | ATLVT012XX665 | 45599 | NSASGGAINDNH | 4.89 | 5.63 | 4.94 |
| 231 | ATLVT012XX2090 | 47024 | SSQSGAATNDNH | 7.52 | 3.94 | 2.58 |
| 232 | ATLVT012XX1240 | 46174 | SGASGGSSNDNA | 6.38 | 3.42 | 5.07 |
| 233 | ATLVT012XX1476 | 46410 | SGQAGGASNDNT | 2.01 | 6.96 | 8.01 |
| 234 | ATLVT012XX1075 | 46009 | NSTSAGSSNDNA | 1.74 | 8.58 | 6.34 |
| 235 | ATLVT012XX2222 | 47156 | SSTSAGATNDNH | 3.93 | 5.98 | 5.91 |
| 236 | ATLVT012XX912 | 45846 | NSQAGGSINDNT | 5.31 | 3.65 | 6.53 |
| 237 | ATLVT012XX3450 | 48384 | TSTTGGSSNDNT | 3.77 | 4.63 | 7.89 |
| 238 | ATLVT012XX2057 | 46991 | SSQAGGSSNDNH | 4.6 | 5.35 | 5.51 |
| 239 | ATLVT012XX1955 | 46889 | SSESGGASNDNH | 3.69 | 8.35 | 3.19 |
| 240 | ATLVT012XX1073 | 46007 | NSTSAGAINDNH | 3.05 | 7.3 | 5.54 |
| 241 | ATLVT012XX565 | 45499 | NGTTGGASNDNA | 4.13 | 4.97 | 6.65 |
| 242 | ATLVT012XX667 | 45601 | NSASGGSSNDNA | 1.52 | 8.52 | 6.55 |
| 243 | ATLVT012XX1245 | 46179 | SGASGGSINDNT | 5.36 | 3.45 | 6.41 |
| 244 | ATLVT012XX2100 | 47034 | SSQSGGASNDNT | 4.85 | 4.68 | 5.67 |
| 245 | ATLVT012XX3355 | 48289 | TSTAGGSINDNA | 3.05 | 4.18 | 9.39 |
| 246 | ATLVT012XX1620 | 46554 | SGTAGGASNDNT | 1.2 | 7.89 | 7.78 |
| 247 | ATLVT012XX3443 | 48377 | TSTTGGASNDNH | 4.94 | 5.09 | 4.87 |
| 248 | ATLVT012XX951 | 45885 | NSQSGGASNDNT | 5.62 | 4.29 | 4.72 |
| 249 | ATLVT012XX3453 | 48387 | TSTTGGSTNDNT | 2.87 | 4.8 | 8.73 |
| 250 | ATLVT012XX1625 | 46559 | SGTAGGSSNDNH | 2.13 | 6.75 | 7.49 |
| 251 | ATLVT012XX1435 | 46369 | SGETGGSINDNA | 5.86 | 4.32 | 4.2 |
| 252 | ATLVT012XX1477 | 46411 | SGQAGGATNDNA | 3.8 | 5.41 | 6.33 |
| 253 | ATLVT012XX1383 | 46317 | SGESGGATNDNT | 5.14 | 5.31 | 4.15 |
| 254 | ATLVT012XX1766 | 46700 | SSAAGGATNDNH | 5.66 | 3.88 | 5.1 |
| 255 | ATLVT012XX2339 | 47273 | TGAAGGASNDNH | 4.43 | 4.81 | 6 |
| 256 | ATLVT012XX1187 | 46121 | SGAAGGASNDNH | 5.38 | 3.72 | 5.75 |
| 257 | ATLVT012XX2101 | 47035 | SSQSGGATNDNA | 4.02 | 5.21 | 6.14 |
| 258 | ATLVT012XX810 | 45744 | NSESGGATNDNT | 2.85 | 7.18 | 5.6 |
| 259 | ATLVT012XX1007 | 45941 | NSQTGGSTNDNH | 6.67 | 5.51 | 1.19 |
| 260 | ATLVT012XX3447 | 48381 | TSTTGGATNDNT | 4.9 | 3.76 | 6.47 |
| 261 | ATLVT012XX1236 | 46170 | SGASGGASNDNT | 3.89 | 6.21 | 4.99 |
| 262 | ATLVT012XX1862 | 46796 | SSATGGATNDNH | 4.95 | 5.64 | 3.88 |
| 263 | ATLVT012XX1117 | 46051 | NSTTAGATNDNA | 3.62 | 6.71 | 4.77 |
| 264 | ATLVT012XX2875 | 47809 | TGTTGGSINDNA | 4.46 | 4.67 | 5.96 |
| 265 | ATLVT012XX811 | 45745 | NSESGGSSNDNA | 7.63 | 3.45 | 2.05 |
| 266 | ATLVT012XX1619 | 46553 | SGTAGGASNDNH | 2.68 | 5.72 | 7.61 |
| 267 | ATLVT012XX1575 | 46509 | SGQTGGAINDNT | 3.06 | 4.83 | 8.1 |
| 268 | ATLVT012XX1912 | 46846 | SSEAGGSSNDNA | 6.88 | 4.26 | 2.05 |
| 269 | ATLVT01XXX1618 | 46552 | SGTAGGASNDNA | 1.28 | 6.96 | 8.08 |
| 270 | ATLVT012XX1197 | 46131 | SGAAGGSINDNT | 4.23 | 4.16 | 6.6 |
| 271 | ATLVT012XX809 | 45743 | NSESGGATNDNH | 6.81 | 3.47 | 3.06 |
| 272 | ATLVT012XX3405 | 48339 | TSTSGGSINDNT | 2.63 | 4.84 | 8.44 |
| 273 | ATLVT012XX1001 | 45935 | NSQTGGATNDNH | 4.66 | 3.42 | 6.76 |
| 274 | ATLVT012XX2196 | 47130 | SSTAGGASNDNT | 2.49 | 2.96 | 11.08 |
| 275 | ATLVT012XX1700 | 46634 | SGTTAGSTNDNH | 4.5 | 4.97 | 5.03 |
| 276 | ATLVT012XX3442 | 48376 | TSTTGGASNDNA | 3.99 | 5.56 | 5.14 |
| 277 | ATLVT012XX720 | 45654 | NSATGGSTNDNT | 3.91 | 4.93 | 5.92 |
| 278 | ATLVT012XX2148 | 47082 | SSQTGGASNDNT | 3.35 | 3.84 | 8.28 |
| 279 | ATLVT012XX2340 | 47274 | TGAAGGASNDNT | 4.15 | 4.41 | 6.16 |
| 280 | ATLVT012XX854 | 45788 | NSETGGASNDNH | 5.87 | 6.66 | 0.3 |
| 281 | ATLVT012XX1578 | 46512 | SGQTGGSSNDNT | 4.35 | 3.34 | 7.13 |
| 282 | ATLVT012XX1646 | 46580 | SGTSAGATNDNH | 4.25 | 4.95 | 5.18 |
| 283 | ATLVT012XX2059 | 46993 | SSQAGGSINDNA | 4.27 | 3.06 | 7.54 |
| 284 | ATLVT012XX2155 | 47089 | SSQTGGSTNDNA | 3.38 | 4.73 | 6.91 |
| 285 | ATLVT012XX2390 | 47324 | TGASGGATNDNH | 4.44 | 5.2 | 4.49 |
| 286 | ATLVT012XX664 | 45598 | NSASGGATNDNA | 3.81 | 6.39 | 3.79 |
| 287 | ATLVT012XX2104 | 47038 | SSQSGGSSNDNA | 3.93 | 5.83 | 4.2 |
| 288 | ATLVT012XX1080 | 46014 | NSTSAGSTNDNT | 3.19 | 4.35 | 7.37 |
| 289 | ATLVT012XX1621 | 46555 | SGTAGGATNDNA | 0.76 | 5.84 | 9.56 |
| 290 | ATLVT012XX3301 | 48235 | TSQTGGATNDNA | 4.83 | 3.27 | 5.9 |
| 291 | ATLVT012XX1286 | 46220 | SGATGGATNDNH | 4.12 | 3.14 | 7.21 |
| 292 | ATLVT012XX953 | 45887 | NSQSGGATNDNH | 6.14 | 3.19 | 3.67 |
| 293 | ATLVT012XX1379 | 46313 | SGESGGASNDNH | 3.5 | 5.29 | 5.42 |
| 294 | ATLVT012XX2240 | 47174 | SSTSGASTNDNH | 6.19 | 2.56 | 4.31 |
| 295 | ATLVT012XX1118 | 46052 | NSTTAGATNDNH | 3.29 | 7.37 | 3.1 |
| 296 | ATLVT012XX89 | 45023 | NGASGGATNDNH | 4.36 | 5.67 | 3.44 |
| 297 | ATLVT012XX1124 | 46058 | NSTTAGSTNDNH | 2.69 | 5.91 | 5.96 |
| 298 | ATLVT012XX3250 | 48184 | TSQSGGASNDNA | 2.22 | 6 | 6.62 |
| 299 | ATLVT012XX1089 | 46023 | NSTSGASTNDNH | 2.37 | 6.15 | 6.12 |
| 300 | ATLVT012XX2098 | 47032 | SSQSGGASNDNA | 3.46 | 5.01 | 5.71 |
| 301 | ATLVT012XX469 | 45403 | NGTAGGASNDNA | 3.43 | 4.5 | 6.4 |
| 302 | ATLVT012XX2051 | 46985 | SSQAGGASNDNH | 4.3 | 3.47 | 6.21 |
| 303 | ATLVT012XX2824 | 47758 | TGTSGGSSNDNA | 4.39 | 3.77 | 5.65 |
| 304 | ATLVT012XX668 | 45602 | NSASGGSSNDNH | 3.13 | 6.01 | 4.92 |
| 305 | ATLVT012XX1334 | 46268 | SGEAGGATNDNH | 2.35 | 5.26 | 6.95 |
| 306 | ATLVT012XX1522 | 46456 | SGQSGGASNDNA | 3.24 | 3.41 | 7.75 |
| 307 | ATLVT012XX3251 | 48185 | TSQSGGASNDNH | 4.8 | 3.78 | 4.53 |
| 308 | ATLVT012XX1114 | 46048 | NSTTAGASNDNA | 4.66 | 5.02 | 3.17 |
| 309 | ATLVT012XX3305 | 48239 | TSQTGGSSNDNH | 3.87 | 5.52 | 3.86 |
| 310 | ATLVT012XX806 | 45740 | NSESGGASNDNH | 6.83 | 2.99 | 2.05 |
| 311 | ATLVT012XX1432 | 46366 | SGETGGSSNDNA | 5.27 | 2.83 | 4.91 |
| 312 | ATLVT012XX1509 | 46443 | SGQSAGSTNDNT | 5.12 | 5.68 | 1.5 |
| 313 | ATLVT012XX143 | 45077 | NGATGGSTNDNH | 6.14 | 3.26 | 2.85 |
| 314 | ATLVT012XX3354 | 48288 | TSTAGGSSNDNT | 1.33 | 6.53 | 6.88 |
| 315 | ATLVT012XX3259 | 48193 | TSQSGGSTNDNA | 5.12 | 4.38 | 3.15 |
| 316 | ATLVT012XX1811 | 46745 | SSASGGASNDNH | 3.72 | 6.12 | 3.27 |
| 317 | ATLVT012XX479 | 45413 | NGTAGGSTNDNH | 3.08 | 4.14 | 6.88 |
| 318 | ATLVT012XX1082 | 46016 | NSTSGAASNDNH | 4.64 | 3.25 | 5.33 |
| 319 | ATLVT012XX1550 | 46484 | SGQTAGATNDNH | 5.34 | 4.48 | 2.56 |
| 320 | ATLVT012XX1115 | 46049 | NSTTAGASNDNH | 4.09 | 4.65 | 4.46 |
| 321 | ATLVT012XX903 | 45837 | NSQAGGASNDNT | 6.75 | 3.74 | 1.06 |
| 322 | ATLVT012XX3352 | 48286 | TSTAGGSSNDNA | 1.64 | 7.01 | 5.59 |
| 323 | ATLVT012XX1234 | 46168 | SGASGGASNDNA | 4.37 | 4.57 | 4.04 |
| 324 | ATLVT012XX473 | 45407 | NGTAGGATNDNH | 4.11 | 2.53 | 7.1 |
| 325 | ATLVT012XX3307 | 48241 | TSQTGGSTNDNA | 3.2 | 4.87 | 5.63 |
| 326 | ATLVT012XX671 | 45605 | NSASGGSTNDNH | 5.23 | 4.1 | 3.11 |
| 327 | ATLVT012XX2818 | 47752 | TGTSGGASNDNA | 4.35 | 3.83 | 4.93 |
| 328 | ATLVT012XX1044 | 45978 | NSTAGASTNDNT | 3.51 | 5.13 | 4.68 |
| 329 | ATLVT012XX1239 | 46173 | SGASGGAINDNT | 4.13 | 3.31 | 5.94 |
| 330 | ATLVT012XX2873 | 47807 | TGTTGGSSNDNH | 5.97 | 2.5 | 3.83 |
| 331 | ATLVT012XX470 | 45404 | NGTAGGASNDNH | 3.86 | 4.57 | 4.74 |
| 332 | ATLVT012XX2298 | 47232 | SSTTGGSSNDNT | 3.07 | 3.2 | 7.77 |
| 333 | ATLVT012XX2338 | 47272 | TGAAGGASNDNA | 3.35 | 4.64 | 5.42 |
| 334 | ATLVT012XX3449 | 48383 | TSTTGGSSNDNH | 5.12 | 2.92 | 4.56 |
| 335 | ATLVT012XX713 | 45647 | NSATGGATNDNH | 3.99 | 3.94 | 5.19 |
| 336 | ATLVT012XX1193 | 46127 | SGAAGGSSNDNH | 2.63 | 3.29 | 8.32 |
| 337 | ATLVT012XX1387 | 46321 | SGESGGSINDNA | 2.99 | 5.73 | 4.56 |
| 338 | ATLVT012XX2820 | 47754 | TGTSGGASNDNT | 4.44 | 3.15 | 5.38 |
| 339 | ATLVT012XX1431 | 46365 | SGETGGATNDNT | 4.95 | 1.7 | 6.35 |
| 340 | ATLVT012XX1005 | 45939 | NSQTGGSSNDNT | 7.55 | 2.9 | 0.21 |
| 341 | ATLVT012XX566 | 45500 | NGTTGGASNDNH | 3.26 | 3.55 | 6.74 |
| 342 | ATLVT012XX2348 | 47282 | TGAAGGSINDNH | 3.03 | 4.7 | 5.62 |
| 343 | ATLVT012XX2872 | 47806 | TGTTGGSSNDNA | 3.47 | 3.99 | 5.78 |
| 344 | ATLVT012XX2375 | 47309 | TGASGAASNDNH | 7.54 | 1.9 | 1.36 |
| 345 | ATLVT012XX1079 | 46013 | NSTSAGSTNDNH | 2.78 | 4.88 | 5.66 |
| 346 | ATLVT012XX2732 | 47666 | TGQTGGSTNDNH | 1.84 | 7.04 | 4.49 |
| 347 | ATLVT012XX2052 | 46986 | SSQAGGASNDNT | 5.18 | 2.02 | 5.13 |
| 348 | ATLVT012XX1772 | 46706 | SSAAGGSINDNH | 5.89 | 3.03 | 2.61 |
| 349 | ATLVT012XX2149 | 47083 | SSQTGGATNDNA | 4.33 | 2.64 | 5.76 |
| 350 | ATLVT012XX336 | 45270 | NGQAGGSTNDNT | 4.7 | 1.49 | 6.58 |
| 351 | ATLVT012XX1817 | 46751 | SSASGGSSNDNH | 4.7 | 5.77 | 1.03 |
| 352 | ATLVT012XX672 | 45606 | NSASGGSINDNT | 2.52 | 6.76 | 3.45 |
| 353 | ATLVT012XX956 | 45890 | NSQSGGSSNDNH | 4.57 | 5.54 | 1.48 |
| 354 | ATLVT012XX1237 | 46171 | SGASGGATNDNA | 4.36 | 3.26 | 4.77 |
| 355 | ATLVT012XX1427 | 46361 | SGETGGASNDNH | 3.96 | 4.56 | 3.72 |
| 356 | ATLVT012XX474 | 45408 | NGTAGGATNDNT | 2.4 | 5.26 | 5.48 |
| 357 | ATLVT012XX2775 | 47709 | TGTAGGATNDNT | 2.22 | 3.32 | 8.27 |
| 358 | ATLVT012XX1694 | 46628 | SGTTAGATNDNH | 4.23 | 3.87 | 4.09 |
| 359 | ATLVT012XX952 | 45886 | NSQSGGATNDNA | 3.69 | 4.79 | 3.84 |
| 360 | ATLVT012XX719 | 45653 | NSATGGSINDNH | 5.06 | 4.58 | 1.72 |
| 361 | ATLVT012XX718 | 45652 | NSATGGSINDNA | 3.7 | 3.98 | 4.81 |
| 362 | ATLVT012XX2973 | 47907 | TSASGGSINDNT | 4.46 | 3.76 | 3.75 |
| 363 | ATLVT012XX1913 | 46847 | SSEAGGSSNDNH | 6.99 | 2.25 | 1.34 |
| 364 | ATLVT012XX2055 | 46989 | SSQAGGATNDNT | 3.16 | 3.26 | 6.6 |
| 365 | ATLVT012XX476 | 45410 | NGTAGGSSNDNH | 2.66 | 4.62 | 5.72 |
| 366 | ATLVT012XX3399 | 48333 | TSTSGGATNDNT | 0.74 | 5.36 | 8.05 |
| 367 | ATLVT012XX2050 | 46984 | SSQAGGASNDNA | 2.84 | 4.65 | 5.35 |
| 368 | ATLVT012XX2199 | 47133 | SSTAGGATNDNT | 1.61 | 3.46 | 8.97 |
| 369 | ATLVT012XX425 | 45359 | NGQTGGATNDNH | 3 | 5.81 | 3.55 |
| 370 | ATLVT012XX568 | 45502 | NGTTGGATNDNA | 2.71 | 4.59 | 5.58 |
| 371 | ATLVT012XX2825 | 47759 | TGTSGGSSNDNH | 5.36 | 2.34 | 3.87 |
| 372 | ATLVT012XX2829 | 47763 | TGTSGGSINDNT | 2.44 | 4.68 | 5.87 |
| 373 | ATLVT012XX2186 | 47120 | SSTAGAATNDNH | 5.5 | 2.4 | 3.54 |
| 374 | ATLVT012XX807 | 45741 | NSESGGASNDNT | 5.24 | 1.23 | 5.47 |
| 375 | ATLVT012XX1087 | 46021 | NSTSGASSNDNH | 1.98 | 5.19 | 5.95 |
| 376 | ATLVT012XX2103 | 47037 | SSQSGGATNDNT | 2.24 | 3.91 | 7.12 |
| 377 | ATLVT012XX957 | 45891 | NSQSGGSSNDNT | 3.71 | 5.26 | 2.83 |
| 378 | ATLVT012XX1502 | 46436 | SGQSAGATNDNH | 5.87 | 3.73 | 1.08 |
| 379 | ATLVT012XX1175 | 46109 | SGAAGAASNDNH | 3 | 3.83 | 5.86 |
| 380 | ATLVT012XX3445 | 48379 | TSTTGGATNDNA | 4.32 | 3.64 | 3.83 |
| 381 | ATLVT012XX1195 | 46129 | SGAAGGSTNDNA | 2.6 | 2.84 | 7.8 |
| 382 | ATLVT012XX816 | 45750 | NSESGGSINDNT | 5.51 | 3.16 | 2.39 |
| 383 | ATLVT012XX2780 | 47714 | IGTAGGSTNDNH | 2.89 | 5.45 | 3.93 |
| 384 | ATLVT012XX1041 | 45975 | NSTAGASSNDNT | 1.23 | 6.65 | 5.23 |
| 385 | ATLVT012XX1120 | 46054 | NSTTAGSSNDNA | 5.39 | 2.75 | 3.07 |
| 386 | ATLVT012XX1083 | 46017 | NSTSGAASNDNT | 3.74 | 2.81 | 5.82 |
| 387 | ATLVT012XX2191 | 47125 | SSTAGASTNDNA | 4.24 | 1.57 | 6.56 |
| 388 | ATLVT012XX2153 | 47087 | SSQTGGSSNDNH | 4.25 | 2.65 | 5.1 |
| 389 | ATLVT012XX621 | 45555 | NSAAGGSSNDNT | 0.92 | 5.84 | 6.7 |
| 390 | ATLVT012XX1031 | 45965 | NSTAAGSINDNH | 4.8 | 2.93 | 3.75 |
| 391 | ATLVT012XX663 | 45597 | NSASGGASNDNT | 3.42 | 3.58 | 5.28 |
| 392 | ATLVT012XX1816 | 46750 | SSASGGSSNDNA | 5.12 | 3.51 | 2.42 |
| 393 | ATLVT012XX622 | 45556 | NSAAGGSINDNA | 3.05 | 3.65 | 5.78 |
| 394 | ATLVT012XX911 | 45845 | NSQAGGSTNDNH | 4.59 | 3.51 | 3.32 |
| 395 | ATLVT012XX1599 | 46533 | SGTAAGATNDNT | 6.49 | 3.34 | 0.25 |
| 396 | ATLVT012XX1072 | 46006 | NSTSAGATNDNA | 3.95 | 3.73 | 4.09 |
| 397 | ATLVT012XX1078 | 46012 | NSTSAGSTNDNA | 2.4 | 4.59 | 5.61 |
| 398 | ATLVT012XX949 | 45883 | NSQSGGASNDNA | 4.97 | 2.68 | 3.66 |
| 399 | ATLVT012XX2867 | 47801 | TGTTGGASNDNH | 1.73 | 4.3 | 7.12 |
| 400 | ATLVT012XX3300 | 48234 | TSQTGGASNDNT | 3.45 | 3.04 | 5.79 |
| 401 | ATLVT012XX670 | 45604 | NSASGGSINDNA | 3.42 | 3.57 | 5.14 |
| 402 | ATLVT012XX1086 | 46020 | NSTSGAATNDNT | 3.13 | 2.77 | 6.67 |
| 403 | ATLVT012XX950 | 45884 | NSQSGGASNDNH | 3.95 | 4.71 | 2.75 |
| 404 | ATLVT012XX1821 | 46755 | SSASGGSTNDNT | 4.73 | 4.32 | 1.87 |
| 405 | ATLVT012XX2966 | 47900 | TSASGGATNDNH | 2.46 | 4.4 | 5.66 |
| 406 | ATLVT012XX1555 | 46489 | SGQTAGSINDNA | 1.9 | 7.57 | 2.48 |
| 407 | ATLVT012XX1186 | 46120 | SGAAGGASNDNA | 2.51 | 3.7 | 6.4 |
| 408 | ATLVT012XX1034 | 45968 | NSTAGAASNDNH | 5.33 | 2.64 | 2.92 |
| 409 | ATLVT012XX862 | 45796 | NSETGGSINDNA | 5.08 | 5.36 | −0.15 |
| 410 | ATLVT012XX2846 | 47780 | TGTTAGATNDNH | 5.67 | 2.05 | 3.06 |
| 411 | ATLVT012XX3386 | 48320 | ISTSGAATNDNH | 1.28 | 5.63 | 5.98 |
| 412 | ATLVT01XXX2798 | 47732 | TGTSAGATNDNH | 4.53 | 2.03 | 5 |
| 413 | ATLVT012XX864 | 45798 | NSETGGSINDNT | 3.56 | 7.35 | −0.18 |
| 414 | ATLVT012XX1859 | 46793 | SSATGGASNDNH | 2.32 | 6.02 | 3.58 |
| 415 | ATLVT012XX1568 | 46502 | SGQTGASINDNH | 3.48 | 4.51 | 3.53 |
| 416 | ATLVT012XX2804 | 47738 | TGTSAGSTNDNH | 6.48 | 1.73 | 1.95 |
| 417 | ATLVT012XX1283 | 46217 | SGATGGASNDNH | 4.37 | 3.19 | 3.68 |
| 418 | ATLVT012XX1433 | 46367 | SGETGGSSNDNH | 4.97 | 2.01 | 4.14 |
| 419 | ATLVT012XX516 | 45450 | NGTSGASTNDNT | 4.61 | 1.7 | 5.16 |
| 420 | ATLVT012XX2003 | 46937 | SSETGGASNDNH | 4.11 | 4.12 | 2.86 |
| 421 | ATLVT012XX95 | 45029 | NGASGGSINDNH | 5.58 | 2.6 | 2.22 |
| 422 | ATLVT012XX1597 | 46531 | SGTAAGATNDNA | 3.99 | 6.13 | 0.37 |
| 423 | ATLVT012XX1508 | 46442 | SGQSAGSTNDNH | 3.4 | 5.34 | 2.39 |
| 424 | ATLVT012XX1572 | 46506 | SGQTGGASNDNT | 2.62 | 2.46 | 7.4 |
| 425 | ATLVT012XX1025 | 45959 | NSTAAGATNDNH | 2.33 | 5.58 | 3.87 |
| 426 | ATLVT012XX1038 | 45972 | NSTAGAATNDNT | 1.99 | 4.43 | 5.92 |
| 427 | ATLVT012XX1030 | 45964 | NSTAAGSINDNA | 5.66 | 1.01 | 4.01 |
| 428 | ATLVT012XX335 | 45269 | NGQAGGSINDNH | 2.58 | 5.95 | 2.92 |
| 429 | ATLVT012XX1964 | 46898 | SSESGGSINDNH | 4.73 | 4.65 | 0.89 |
| 430 | ATLVT012XX2864 | 47798 | TGTIGASINDNH | 3.67 | 3.54 | 4.09 |
| 431 | ATLVT012XX1810 | 46744 | SSASGGASNDNA | 4.55 | 3.88 | 2.14 |
| 432 | ATLVT012XX1004 | 45938 | NSQTGGSSNDNH | 4.97 | 5.15 | −0.22 |
| 433 | ATLVT012XX383 | 45317 | NGQSGGSTNDNH | 1.83 | 6.48 | 3.41 |
| 434 | ATLVT012XX2005 | 46939 | SSETGGATNDNA | 4.72 | 4.35 | 1.16 |
| 435 | ATLVT012XX2779 | 47713 | TGTAGGSTNDNA | 2.62 | 2.51 | 7.13 |
| 436 | ATLVT012XX1242 | 46176 | SGASGGSSNDNT | 3.56 | 2.87 | 5.03 |
| 437 | ATLVT012XX1116 | 46050 | NSTTAGASNDNT | 5.24 | 2.85 | 2.18 |
| 438 | ATLVT012XX968 | 45902 | NSQTAASSNDNH | 2.97 | 1.57 | 7.69 |
| 439 | ATLVT012XX2776 | 47710 | TGTAGGSSNDNA | 2.54 | 3.49 | 5.93 |
| 440 | ATLVT012XX483 | 45417 | NGTSAAASNDNT | 2.18 | 3.47 | 6.56 |
| 441 | ATLVT012XX377 | 45311 | NGQSGGATNDNH | 3.99 | 2.7 | 4.42 |
| 442 | ATLVT012XX3212 | 48146 | TSQAGGSTNDNH | 3.47 | 1.3 | 7.1 |
| 443 | ATLVT012XX1434 | 46368 | SGETGGSSNDNT | 3.7 | 3.13 | 4.34 |
| 444 | ATLVT012XX1813 | 46747 | SSASGGATNDNA | 3.05 | 5.62 | 2.21 |
| 445 | ATLVT012XX2821 | 47755 | TGTSGGATNDNA | 4.19 | 3.19 | 3.37 |
| 446 | ATLVT012XX1039 | 45973 | NSTAGASSNDNA | 2.51 | 4.98 | 3.94 |
| 447 | ATLVT012XX1482 | 46416 | SGQAGGSSNDNT | 2.16 | 2.59 | 7.58 |
| 448 | ATLVT012XX1287 | 46221 | SGATGGATNDNT | 4.22 | 1.52 | 5.43 |
| 449 | ATLVT012XX3306 | 48240 | TSQTGGSSNDNT | 4.59 | 2.43 | 3.62 |
| 450 | ATLVT012XX1029 | 45963 | NSTAAGSSNDNT | 4.73 | 1.53 | 4.5 |
| 451 | ATLVT012XX2154 | 47088 | SSQTGGSSNDNT | 3.22 | 2.93 | 5.29 |
| 452 | ATLVT012XX1412 | 46346 | SGETAGSINDNH | 2.36 | 4.62 | 4.55 |
| 453 | ATLVT012XX2289 | 47223 | SSTTGASTNDNT | 4.93 | 2.1 | 3.39 |
| 454 | ATLVT012XX2392 | 47326 | TGASGGSSNDNA | 2.89 | 1.96 | 7.01 |
| 455 | ATLVT012XX2877 | 47811 | TGTTGGSTNDNT | 2.16 | 3.68 | 6.03 |
| 456 | ATLVT012XX905 | 45839 | NSQAGGAINDNH | 3.74 | 3.72 | 3.25 |
| 457 | ATLVT012XX139 | 45073 | NGATGGSSNDNA | 5.12 | 1.53 | 3.71 |
| 458 | ATLVT012XX1812 | 46746 | SSASGGASNDNT | 3.73 | 4.83 | 1.83 |
| 459 | ATLVT012XX814 | 45748 | NSESGGSINDNA | 6.24 | 1.55 | 1.73 |
| 460 | ATLVT012XX2923 | 47857 | TSAAGGSTNDNA | 1.85 | 6.43 | 2.97 |
| 461 | ATLVT012XX1658 | 46592 | SGTSGAATNDNH | 3.57 | 3.3 | 4.03 |
| 462 | ATLVT012XX239 | 45173 | NGESGGSTNDNH | 2.87 | 7.07 | 0.38 |
| 463 | ATLVT012XX1429 | 46363 | SGETGGAINDNA | 2.79 | 4.56 | 3.74 |
| 464 | ATLVT012XX1189 | 46123 | SGAAGGATNDNA | 1.82 | 3.44 | 6.84 |
| 465 | ATLVT012XX1754 | 46688 | SSAAGAATNDNH | 2.9 | 4.47 | 3.62 |
| 466 | ATLVT012XX2342 | 47276 | TGAAGGATNDNH | 2.32 | 3.18 | 6.25 |
| 467 | ATLVT012XX908 | 45842 | NSQAGGSSNDNH | 4.98 | 3.99 | 0.65 |
| 468 | ATLVT012XX2731 | 47665 | TGQTGGSTNDNA | 1.49 | 3.8 | 6.87 |
| 469 | ATLVT012XX716 | 45650 | NSATGGSSNDNH | 3.6] | 5.12 | 1.5 |
| 470 | ATLVT012XX1965 | 46899 | SSESGGSINDNT | 4.89 | 4.55 | 0 |
| 471 | ATLVT012XX2297 | 47231 | SSTTGGSSNDNH | 3.35 | 3.92 | 3.42 |
| 472 | ATLVT012XX1071 | 46005 | NSTSAGASNDNI | 2.57 | 3.86 | 4.83 |
| 473 | ATLVT012XX662 | 45596 | NSASGGASNDNH | 4.56 | 4.01 | 1.18 |
| 474 | ATLVT012XX2969 | 47903 | TSASGGSSNDNH | 5.15 | 2.25 | 2.43 |
| 475 | ATLVT012XX813 | 45747 | NSESGGSSNDNT | 4.82 | 2.81 | 2.24 |
| 476 | ATLVT012XX2287 | 47221 | SSTTGASTNDNA | 3.52 | 5.39 | 1.15 |
| 477 | ATLVT012XX2056 | 46990 | SSQAGGSSNDNA | 2.84 | 1.87 | 6.83 |
| 478 | ATLVT012XX1661 | 46595 | SGTSGASSNDNH | 3.2 | 1.26 | 6.94 |
| 479 | ATLVT012XX602 | 45536 | NSAAGAASNDNH | 3.35 | 3.56 | 3.73 |
| 480 | ATLVT012XX515 | 45449 | NGTSGASTNDNH | 2.45 | 3.02 | 5.96 |
| 481 | ATLVT012XX2968 | 47902 | TSASGGSSNDNA | 4.25 | 0.88 | 5.62 |
| 482 | ATLVT012XX545 | 45479 | NGTTAGATNDNH | 5.73 | 1.16 | 2.73 |
| 483 | ATLVT012XX1340 | 46274 | SGEAGGSTNDNH | 4.51 | 2.49 | 3.09 |
| 484 | ATLVT012XX2678 | 47612 | TGQSGGATNDNH | 3.15 | 2.84 | 4.95 |
| 485 | ATLVT012XX2777 | 47711 | TGTAGGSSNDNH | 3.51 | 2.69 | 4.52 |
| 486 | ATLVT012XX1381 | 46315 | SGESGGATNDNA | 3.87 | 1.62 | 5.27 |
| 487 | ATLVT012XX2282 | 47216 | SSTTGAATNDNH | 4.89 | 2.68 | 2.16 |
| 488 | ATLVT012XX2239 | 47173 | SSTSGASINDNA | 3.61 | 3.87 | 2.82 |
| 489 | ATLVT012XX2924 | 47858 | TSAAGGSTNDNH | 2.57 | 4.56 | 3.71 |
| 490 | ATLVT012XX1188 | 46122 | SGAAGGASNDNT | 1.87 | 3.58 | 6.16 |
| 491 | ATLVT012XX2729 | 47663 | TGQTGGSSNDNH | 3.83 | 3.71 | 2.62 |
| 492 | ATLVT012XX2971 | 47905 | TSASGGSTNDNA | 3.88 | 3.15 | 3.27 |
| 493 | ATLVT012XX2157 | 47091 | SSQTGGSTNDNT | 2.32 | 3.63 | 5.28 |
| 494 | ATLVT012XX2630 | 47564 | TGQAGGATNDNH | 2.92 | 3.57 | 4.33 |
| 495 | ATLVT012XX1037 | 45971 | NSTAGAATNDNH | 3.88 | 3.18 | 3.17 |
| 496 | ATLVT012XX2007 | 46941 | SSETGGATNDNT | 4.08 | 2.97 | 3.09 |
| 497 | ATLVT012XX1194 | 46128 | SGAAGGSSNDNT | 1.11 | 3.79 | 7.11 |
| 498 | ATLVT012XX2234 | 47168 | SSTSGAATNDNH | 3.6 | 2.84 | 4.06 |
| 499 | ATLVT012XX1069 | 46003 | NSTSAGASNDNA | 1.53 | 6.11 | 3.37 |
| 500 | ATLVT012XX1288 | 46222 | SGATGGSSNDNA | 4.45 | 2.4 | 3.13 |
| 501 | ATLVT012XX815 | 45749 | NSESGGSINDNH | 4.46 | 4.04 | 1 |
| 502 | ATLVT012XX1214 | 46148 | SGASAGATNDNH | 4.35 | 2.72 | 2.87 |
| 503 | ATLVT012XX1184 | 46118 | SGAAGASINDNH | 2.9 | 4.22 | 3.39 |
| 504 | ATLVT012XX1496 | 46430 | SGQSAASINDNH | 3.71 | 4.54 | 1.58 |
| 505 | ATLVT012XX2606 | 47540 | TGQAAGATNDNH | 2.52 | 4.01 | 4.27 |
| 506 | ATLVT012XX2233 | 47167 | SSTSGAATNDNA | 3.15 | 3.3 | 4.08 |
| 507 | ATLVT012XX1074 | 46008 | NSTSAGAINDNT | 3.38 | 2.79 | 4.35 |
| 508 | ATLVT012XX2957 | 47891 | TSASGASSNDNH | 6.29 | 0.43 | 2.33 |
| 509 | ATLVT012XX860 | 45794 | NSETGGSSNDNH | 4.99 | 3.45 | 0.64 |
| 510 | ATLVT012XX1289 | 46223 | SGATGGSSNDNH | 5.61 | 1.59 | 1.97 |
| 511 | ATLVT012XX669 | 45603 | NSASGGSSNDNT | 2.14 | 3.73 | 5.17 |
| 512 | ATLVT012XX605 | 45539 | NSAAGAATNDNH | 3.07 | 3.52 | 3.84 |
| 513 | ATLVT012XX812 | 45746 | NSESGGSSNDNH | 4.41 | 3.63 | 1.35 |
| 514 | ATLVT012XX3298 | 48232 | TSQTGGASNDNA | 1.55 | 5.16 | 4.27 |
| 515 | ATLVT012XX2582 | 47516 | TGETGGATNDNH | 4.03 | 3.13 | 2.63 |
| 516 | ATLVT012XX551 | 45485 | NGTTAGSTNDNH | 2.57 | 2.91 | 5.38 |
| 517 | ATLVT012XX557 | 45491 | NGTTGAATNDNH | 4.51 | 2.53 | 2.55 |
| 518 | ATLVT012XX2106 | 47040 | SSQSGGSSNDNT | 3.21 | 2.84 | 4.36 |
| 519 | ATLVT012XX1556 | 46490 | SGQTAGSTNDNH | 2.41 | 4.09 | 4.13 |
| 520 | ATLVT012XX3304 | 48238 | TSQTGGSSNDNA | 3.24 | 3.2 | 3.84 |
| 521 | ATLVT012XX2778 | 47712 | TGTAGGSSNDNT | 1.77 | 3.61 | 5.82 |
| 522 | ATLVT012XX1437 | 46371 | SGETGGSTNDNT | 4.5 | 1.2 | 4.23 |
| 523 | ATLVT012XX2587 | 47521 | TGETGGSINDNA | 4.8 | 3.03 | 1.3 |
| 524 | ATLVT012XX391 | 45325 | NGQTAASSNDNA | 4.86 | −0.25 | 5.41 |
| 525 | ATLVT012XX1520 | 46454 | SGQSGASTNDNH | 1.15 | 7.24 | 2.13 |
| 526 | ATLVT012XX2138 | 47072 | SSQTGAATNDNH | 2.36 | 0.76 | 8.38 |
| 527 | ATLVT012XX1907 | 46841 | SSEAGGASNDNH | 2.89 | 5.29 | 1.52 |
| 528 | ATLVT012XX3356 | 48290 | TSTAGGSINDNH | 2.39 | 3.51 | 4.67 |
| 529 | ATLVT012XX1132 | 46066 | NSTTGASSNDNA | 1.24 | 5.02 | 4.7 |
| 530 | ATLVT012XX1644 | 46578 | SGTSAGASNDNT | 4.95 | 0.48 | 4.16 |
| 531 | ATLVT012XX853 | 45787 | NSETGGASNDNA | 1.47 | 6.62 | 2.19 |
| 532 | ATLVT012XX3206 | 48140 | TSQAGGATNDNH | 1.74 | 3.64 | 5.54 |
| 533 | ATLVT012XX1428 | 46362 | SGETGGASNDNT | 4.7 | 0.95 | 3.89 |
| 534 | ATLVT012XX1081 | 46015 | NSTSGAASNDNA | 3.74 | 1.34 | 5.04 |
| 535 | ATLVT012XX1076 | 46010 | NSTSAGSSNDNH | 1.67 | 4.46 | 4.55 |
| 536 | ATLVT012XX2030 | 46964 | SSQAAGATNDNH | 2.24 | 4.25 | 3.85 |
| 537 | ATLVT012XX1915 | 46849 | SSEAGGSINDNA | 4.76 | 2.6 | 1.64 |
| 538 | ATLVT012XX1610 | 46544 | SGTAGAATNDNH | 2.57 | 3.55 | 4.17 |
| 539 | ATLVT012XX2826 | 47760 | TGTSGGSSNDNT | 3.17 | 1.26 | 6.06 |
| 540 | ATLVT012XX2284 | 47218 | SSTTGASSNDNA | 2.41 | 6.71 | 0.36 |
| 541 | ATLVT012XX1119 | 46053 | NSTTAGATNDNT | 1.47 | 4.61 | 4.67 |
| 542 | ATLVT012XX1123 | 46057 | NSTTAGSINDNA | 1.29 | 3.69 | 6.15 |
| 543 | ATLVT012XX1131 | 46065 | NSTTGAATNDNT | 1.82 | 3.53 | 5.43 |
| 544 | ATLVT012XX1211 | 46145 | SGASAGASNDNH | 5.03 | 2.83 | 0.82 |
| 545 | ATLVT012XX1521 | 46455 | SGQSGASTNDNT | 3.76 | 2.56 | 3.35 |
| 546 | ATLVT012XX378 | 45312 | NGQSGGATNDNT | 3.81 | 1.68 | 4.36 |
| 547 | ATLVT012XX119 | 45053 | NGATAGSINDNH | 6.79 | 0.72 | 0.46 |
| 548 | ATLVT012XX1708 | 46642 | SGTTGASSNDNA | 4.91 | 1.72 | 2.39 |
| 549 | ATLVT012XX2438 | 47372 | TGATGGATNDNH | 2.66 | 2.83 | 4.8 |
| 550 | ATLVT012XX1963 | 46897 | SSESGGSINDNA | 4.88 | 3.21 | 0.5 |
| 551 | ATLVT012XX2772 | 47706 | TGTAGGASNDNT | 2.61 | 2.43 | 5.36 |
| 552 | ATLVT012XX1122 | 46056 | NSTTAGSSNDNT | 1.92 | 3.38 | 5.32 |
| 553 | ATLVT012XX1389 | 46323 | SGESGGSINDNT | 3.33 | 3.08 | 3.25 |
| 554 | ATLVT012XX1032 | 45966 | NSTAAGSINDNT | 3.62 | 2 | 4.13 |
| 555 | ATLVT012XX2750 | 47684 | TGTAAGATNDNH | 2.32 | 5.21 | 2.16 |
| 556 | ATLVT012XX137 | 45071 | NGATGGATNDNH | 3.23 | 2.11 | 4.56 |
| 557 | ATLVT012XX1665 | 46599 | SGTSGASTNDNT | 4.67 | 1.62 | 2.73 |
| 558 | ATLVT012XX509 | 45443 | NGTSGAATNDNH | 5.09 | 1.61 | 1.98 |
| 559 | ATLVT012XX954 | 45888 | NSQSGGATNDNT | 4.05 | 2.41 | 2.72 |
| 560 | ATLVT012XX1514 | 46448 | SGQSGAATNDNH | 3.13 | 3.65 | 2.71 |
| 561 | ATLVT012XX879 | 45813 | NSQAAGASNDNT | 2.66 | 3.5 | 3.7 |
| 562 | ATLVT012XX251 | 45185 | NGETAASTNDNH | 4.09 | 5.38 | −1.18 |
| 563 | ATLVT012XX3353 | 48287 | TSTAGGSENDNH | 3.2 | 3.31 | 2.99 |
| 564 | ATLVT012XX2843 | 47777 | TGTTAGASNDNH | 4.56 | 1.7 | 2.72 |
| 565 | ATLVT012XX805 | 45739 | NSESGGASNDNA | 5.53 | 0.79 | 2.22 |
| 566 | ATLVT012XX2868 | 47802 | TGTTGGASNDNT | 2.26 | 2.21 | 5.99 |
| 567 | ATLVT012XX1042 | 45976 | NSTAGASTNDNA | 2.06 | 2.81 | 5.53 |
| 568 | ATLVT012XX2871 | 47805 | TGTTGGATNDNT | 1.56 | 3.6 | 5.34 |
| 569 | ATLVT012XX2627 | 47561 | TGQAGGASNDNH | 4.14 | 0.94 | 4.32 |
| 570 | ATLVT012XX1914 | 46848 | SSEAGGSSNDNT | 4.93 | 1.29 | 2.53 |
| 571 | ATLVT012XX1370 | 46304 | SGESGAATNDNH | 0.69 | 7.59 | 1.67 |
| 572 | ATLVT012XX1384 | 46318 | SGESGGSSNDNA | 4.08 | 2.22 | 2.68 |
| 573 | ATLVT012XX3357 | 48291 | TSTAGGSINDNT | 1.39 | 2.46 | 6.94 |
| 574 | ATLVT012XX2636 | 47570 | TGQAGGSINDNH | 1.72 | 3.06 | 5.57 |
| 575 | ATLVT012XX1103 | 46037 | NSTTAAASNDNH | 2.77 | 4.39 | 2.06 |
| 576 | ATLVT012XX1129 | 46063 | NSTTGAATNDNA | 1.31 | 4.17 | 4.82 |
| 577 | ATLVT012XX3326 | 48260 | TSTAAGAINDNH | 0.15 | 8.74 | 0.93 |
| 578 | ATLVT012XX1643 | 46577 | SGTSAGASNDNH | 4.16 | 1.39 | 3.52 |
| 579 | ATLVT012XX2042 | 46976 | SSQAGAATNDNH | 2.74 | 2.49 | 4.5 |
| 580 | ATLVT012XX1215 | 46149 | SGASAGATNDNT | 3.43 | 1.52 | 4.53 |
| 581 | ATLVT012XX1192 | 46126 | SGAAGGSSNDNA | 1.62 | 1.95 | 7.07 |
| 582 | ATLVT012XX1906 | 46840 | SSEAGGASNDNA | 4.1 | 2.19 | 2.5 |
| 583 | ATLVT012XX2095 | 47029 | SSQSGASTNDNA | 4.15 | 0.41 | 4.7 |
| 584 | ATLVT012XX2152 | 47086 | SSQTGGSSNDNA | 2.39 | 1.3 | 6.57 |
| 585 | ATLVT012XX1008 | 45942 | NSQTGGSTNDNT | 2.86 | 5.16 | 0.79 |
| 586 | ATLVT012XX1336 | 46270 | SGEAGGSSNDNA | 3.59 | 4.35 | 0.58 |
| 587 | ATLVT012XX2393 | 47327 | TGASGGSSNDNH | 1.6 | 1.69 | 7.37 |
| 588 | ATLVT012XX3347 | 48281 | TSTAGGASNDNH | 2.13 | 3.47 | 4.17 |
| 589 | ATLVT012XX2823 | 47757 | TGTSGGATNDNT | 1.47 | 2.91 | 6.01 |
| 590 | ATLVT012XX1088 | 46022 | NSTSGASSNDNT | 1.39 | 3.34 | 5.59 |
| 591 | ATLVT012XX1134 | 46068 | NSTTGASSNDNT | 0.4 | 3.92 | 6.53 |
| 592 | ATLVT012XX2139 | 47073 | SSQTGAATNDNT | 2.74 | −1.39 | 9.35 |
| 593 | ATLVT012XX2874 | 47808 | TGTTGGSSNDNT | 1.87 | 2.74 | 5.5 |
| 594 | ATLVT012XX1003 | 45937 | NSQTGGSSNDNA | 4.78 | 2.23 | 1.13 |
| 595 | ATLVT012XX1035 | 45969 | NSTAGAASNDNT | 3.06 | 1.96 | 4.43 |
| 596 | ATLVT012XX1183 | 46117 | SGAAGASINDNA | 1.27 | 5.11 | 3.43 |
| 597 | ATLVT012XX661 | 45595 | NSASGGASNDNA | 2.82 | 1.92 | 4.86 |
| 598 | ATLVT012XX2894 | 47828 | TSAAAGAINDNH | 4.38 | 2.3 | 1.69 |
| 599 | ATLVT012XX2728 | 47662 | TGQTGGSSNDNA | 1.72 | 4.75 | 3.07 |
| 600 | ATLVT012XX2852 | 47786 | TGTTAGSINDNH | 2.16 | 1.24 | 6.83 |
| 601 | ATLVT012XX2723 | 47657 | TGQTGGASNDNH | 2.04 | 1.58 | 6.58 |
| 602 | ATLVT012XX1712 | 46646 | SGTTGASTNDNH | 5.33 | 0.8 | 1.95 |
| 603 | ATLVT012XX1909 | 46843 | SSEAGGATNDNA | 3.44 | 1.9 | 3.75 |
| 604 | ATLVT012XX2315 | 47249 | TGAAAGASNDNH | 5.08 | 1.23 | 1.79 |
| 605 | ATLVT012XX941 | 45875 | NSQSGAATNDNH | 4.54 | 2.59 | 0.96 |
| 606 | ATLVT012XX1701 | 46635 | SGTTAGSINDNT | 0.04 | 4.83 | 5.78 |
| 607 | ATLVT012XX2330 | 47264 | TGAAGAATNDNH | 2.04 | 3.48 | 4.09 |
| 608 | ATLVT012XX3348 | 48282 | TSTAGGASNDNT | 1.12 | 3.3 | 5.88 |
| 609 | ATLVT012XX233 | 45167 | NGESGGATNDNH | 4.23 | 3.07 | 0.84 |
| 610 | ATLVT012XX863 | 45797 | NSETGGSINDNH | 2.37 | 6.53 | −0.42 |
| 611 | ATLVT012XX321 | 45255 | NGQAGASSNDNT | 4.99 | −1.11 | 4.88 |
| 612 | ATLVT012XX2096 | 47030 | SSQSGASTNDNH | 3.8 | 2.84 | 1.82 |
| 613 | ATLVT012XX1231 | 46165 | SGASGASINDNA | 5.17 | 0.62 | 2.31 |
| 614 | ATLVT012XX1070 | 46004 | NSTSAGASNDNH | 1.09 | 4.42 | 4.4 |
| 615 | ATLVT012XX2725 | 47659 | TGQTGGATNDNA | 3.88 | −0.09 | 5.4 |
| 616 | ATLVT012XX1385 | 46319 | SGESGGSSNDNH | 3.59 | 2.73 | 2.26 |
| 617 | ATLVT012XX1982 | 46916 | SSETAGAINDNH | 3.42 | 4.18 | 0.67 |
| 618 | ATLVT012XX2396 | 47330 | TGASGGSINDNH | 3.18 | 2.25 | 3.55 |
| 619 | ATLVT012XX2008 | 46942 | SSETGGSSNDNA | 4.09 | 2.17 | 2.07 |
| 620 | ATLVT012XX3284 | 48218 | TSQTAGSINDNH | 1.49 | 5.58 | 2.12 |
| 621 | ATLVT012XX1284 | 46218 | SGATGGASNDNT | 2.18 | 2.86 | 4.42 |
| 622 | ATLVT012XX1137 | 46071 | NSTTGASINDNT | 1.66 | 2.46 | 5.81 |
| 623 | ATLVT012XX2241 | 47175 | SSTSGASINDNT | 4.28 | 1.29 | 2.82 |
| 624 | ATLVT012XX623 | 45557 | NSAAGGSTNDNH | 1.28 | 6.44 | 1.32 |
| 625 | ATLVT012XX2639 | 47573 | TGQSAAASNDNH | 4.08 | −0.11 | 4.91 |
| 626 | ATLVT012XX2326 | 47260 | TGAAGAASNDNA | 1.96 | 3.36 | 4.09 |
| 627 | ATLVT012XX543 | 45477 | NGTTAGASNDNT | 2.73 | 4.29 | 1.55 |
| 628 | ATLVT012XX947 | 45881 | NSQSGASTNDNH | 2.89 | 5.68 | −0.52 |
| 629 | ATLVT012XX939 | 45873 | NSQSGAASNDNT | 2.2 | 4.57 | 2.1 |
| 630 | ATLVT012XX1406 | 46340 | SGETAGATNDNH | 2.09 | 5.89 | 0.56 |
| 631 | ATLVT012XX3434 | 48368 | TSTTGAATNDNH | 3.66 | 2.15 | 2.64 |
| 632 | ATLVT012XX861 | 45795 | NSETGGSSNDNT | 2.99 | 2.22 | 3.69 |
| 633 | ATLVT012XX857 | 45791 | NSETGGATNDNH | 3.29 | 3.67 | 1.28 |
| 634 | ATLVT012XX332 | 45266 | NGQAGGSSNDNH | 4.69 | 1.93 | 1.11 |
| 635 | ATLVT012XX2288 | 47222 | SSTTGASTNDNH | 4.45 | 2.54 | 0.73 |
| 636 | ATLVT012XX1426 | 46360 | SGETGGASNDNA | 2.63 | 2.36 | 4.04 |
| 637 | ATLVT012XX2869 | 47803 | TGTTGGATNDNA | 0.8 | 3.75 | 5.39 |
| 638 | ATLVT012XX938 | 45872 | NSQSGAASNDNH | 2.69 | 4.2 | 1.55 |
| 639 | ATLVT012XX3245 | 48179 | TSQSGASSNDNH | 2 | 4.92 | 1.81 |
| 640 | ATLVT012XX2684 | 47618 | TGQSGGSINDNH | 1.06 | 4.95 | 3.38 |
| 641 | ATLVT012XX619 | 45553 | NSAAGGSSNDNA | 1.03 | 3.8 | 4.9 |
| 642 | ATLVT012XX2329 | 47263 | TGAAGAATNDNA | 1.9 | 3.25 | 4.08 |
| 643 | ATLVT012XX701 | 45635 | NSATGAATNDNH | 5 | 1.28 | 1.29 |
| 644 | ATLVT012XX1006 | 45940 | NSQTGGSTNDNA | 4.38 | 2.44 | 0.87 |
| 645 | ATLVT012XX2534 | 47468 | TGESGGATNDNH | 2.32 | 3.33 | 3.19 |
| 646 | ATLVT012XX899 | 45833 | NSQAGASTNDNH | 2.3 | 5.01 | 1.06 |
| 647 | ATLVT012XX1967 | 46901 | SSETAAASNDNH | −0.44 | 4.33 | 6.63 |
| 648 | ATLVT012XX711 | 45645 | NSATGGASNDNT | 2.21 | 3.24 | 3.48 |
| 649 | ATLVT012XX717 | 45651 | NSATGGSSNDNT | 2.89 | 1.21 | 4.92 |
| 650 | ATLVT012XX3253 | 48187 | TSQSGGATNDNA | 2.96 | 2.57 | 3.04 |
| 651 | ATLVT012XX1495 | 46429 | SGQSAASTNDNA | 1.8 | 6.72 | −0.31 |
| 652 | ATLVT012XX1191 | 46125 | SGAAGGATNDNT | 0.81 | 3.4 | 5.65 |
| 653 | ATLVT012XX1868 | 46802 | SSATGGSINDNH | 5.56 | 0.85 | 0.78 |
| 654 | ATLVT012XX44 | 44978 | NGAAGGSSNDNH | 1.91 | 3.19 | 4.02 |
| 655 | ATLVT012XX1898 | 46832 | SSEAGAATNDNH | 2.19 | 4.45 | 1.91 |
| 656 | ATLVT012XX910 | 45844 | NSQAGGSTNDNA | 2.87 | 3.57 | 1.87 |
| 657 | ATLVT012XX998 | 45932 | NSQTGGASNDNH | 2.97 | 2.85 | 2.64 |
| 658 | ATLVT012XX2794 | 47728 | TGTSAGASNDNA | 4.87 | 0.85 | 1.95 |
| 659 | ATLVT012XX1858 | 46792 | SSATGGASNDNA | 5.07 | 1.58 | 0.65 |
| 660 | ATLVT012XX3303 | 48237 | TSQTGGATNDNT | 0.93 | 2.69 | 6.3 |
| 661 | ATLVT012XX2231 | 47165 | SSTSGAASNDNH | 2.92 | 2.11 | 3.62 |
| 662 | ATLVT012XX2228 | 47162 | SSTSAGSINDNH | 3.11 | 2.7 | 2.53 |
| 663 | ATLVT012XX909 | 45843 | NSQAGGSSNDNT | 3.24 | 2.36 | 2.72 |
| 664 | ATLVT012XX2236 | 47170 | SSTSGASSNDNA | 4.44 | 1.33 | 1.98 |
| 665 | ATLVT012XX2918 | 47852 | TSAAGGATNDNH | 2.89 | 2.96 | 2.53 |
| 666 | ATLVT012XX1519 | 46453 | SGQSGASTNDNA | 2.65 | 2.91 | 3 |
| 667 | ATLVT012XX1043 | 45977 | NSTAGASINDNH | 2.02 | 3.58 | 3.21 |
| 668 | ATLVT012XX384 | 45318 | NGQSGGSINDNT | 1.66 | 4.18 | 3.05 |
| 669 | ATLVT012XX333 | 45267 | NGQAGGSSNDNT | 1.72 | 3.96 | 3.16 |
| 670 | ATLVT012XX902 | 45836 | NSQAGGASNDNH | 4.13 | 1.98 | 1.59 |
| 671 | ATLVT012XX1285 | 46219 | SGATGGATNDNA | 1.65 | 2.13 | 5.6 |
| 672 | ATLVT012XX1917 | 46851 | SSEAGGSINDNT | 3.65 | 2.39 | 1.83 |
| 673 | ATLVT012XX2866 | 47800 | TGTTGGASNDNA | 1.56 | 2.73 | 4.97 |
| 674 | ATLVT012XX715 | 45649 | NSATGGSSNDNA | 2.53 | 2.34 | 3.79 |
| 675 | ATLVT012XX2237 | 47171 | SSTSGASSNDNH | 2.3 | 3.92 | 2.15 |
| 676 | ATLVT012XX642 | 45576 | NSASAGAINDNT | 2.65 | 5.09 | 0.04 |
| 677 | ATLVT012XX2752 | 47686 | TGTAAGSSNDNA | 4.94 | 1.12 | 1.22 |
| 678 | ATLVT012XX3309 | 48243 | TSQTGGSINDNT | 1.43 | 3.8 | 3.77 |
| 679 | ATLVT012XX91 | 45025 | NGASGGSSNDNA | 2.52 | 1.76 | 4.48 |
| 680 | ATLVT012XX808 | 45742 | NSESGGATNDNA | 3.12 | 2.7 | 2.23 |
| 681 | ATLVT012XX2905 | 47839 | TSAAGAATNDNA | 1.97 | 3.11 | 3.67 |
| 682 | ATLVT012XX2453 | 47387 | TGEAAASSNDNH | 4.37 | 2.42 | 0.44 |
| 683 | ATLVT012XX1867 | 46801 | SSATGGSINDNA | 4.01 | 2.3 | 1.21 |
| 684 | ATLVT012XX1066 | 46000 | NSTSAASTNDNA | 2.15 | 3.11 | 3.37 |
| 685 | ATLVT012XX113 | 45047 | NGATAGATNDNH | 3.78 | 3.44 | 0.14 |
| 686 | ATLVT012XX1133 | 46067 | NSTTGASSNDNH | 1.14 | 2.59 | 5.75 |
| 687 | ATLVT012XX1790 | 46724 | SSASAGAINDNH | 2.86 | 5.12 | −0.47 |
| 688 | ATLVT012XX643 | 45577 | NSASAGSSNDNA | 0.04 | 4.83 | 4.74 |
| 689 | ATLVT012XX2387 | 47321 | TGASGGASNDNH | 3.09 | 1.9 | 3.27 |
| 690 | ATLVT012XX1291 | 46225 | SGATGGSINDNA | 3.07 | 2.41 | 2.65 |
| 691 | ATLVT012XX1135 | 46069 | NSTTGASTNDNA | 0.21 | 4.05 | 5.39 |
| 692 | ATLVT012XX1601 | 46535 | SGTAAGSSNDNH | 2.96 | 4.64 | −0.08 |
| 693 | ATLVT012XX1565 | 46499 | SGQTGASSNDNH | 2.75 | 3.61 | 1.6 |
| 694 | ATLVT012XX1110 | 46044 | NSTTAASSNDNT | 4.61 | −0.26 | 3.37 |
| 695 | ATLVT012XX2220 | 47154 | SSTSAGASNDNT | 3.21 | 0.55 | 4.7 |
| 696 | ATLVT012XX318 | 45252 | NGQAGAATNDNT | 6 | −0.44 | 1.16 |
| 697 | ATLVT012XX3441 | 48375 | TSTTGASINDNI | 1.27 | 2.66 | 5.29 |
| 698 | ATLVT012XX2184 | 47118 | SSTAGAASNDNT | 3.72 | 1.82 | 2.15 |
| 699 | ATLVT012XX497 | 45431 | NGTSAGATNDNH | 2.81 | 2.75 | 2.49 |
| 700 | ATLVT012XX314 | 45448 | NGTSGASINDNA | 1.3 | 1.86 | 6.2 |
| 701 | ATLVT012XX2771 | 47705 | TGTAGGASNDNH | 2.02 | 1.92 | 4.87 |
| 702 | ATLVT012XX2906 | 47840 | TSAAGAATNDNH | 2.1 | 2.96 | 3.37 |
| 703 | ATLVT01XXX1166 | 46100 | SGAAAGAINDNH | 1.81 | 2.85 | 4 |
| 704 | ATLVT012XX765 | 45699 | NSEAGGSSNDNT | 2.25 | 1.02 | 5.59 |
| 705 | ATLVT012XX932 | 45866 | NSQSAGSSNDNH | 3.47 | 4.62 | −1.13 |
| 706 | ATLVT012XX2817 | 47751 | TGTSGASTNDNT | 0.4 | 7.81 | 0.03 |
| 707 | ATLVT012XX472 | 45406 | NGTAGGAINDNA | 1.98 | 1.69 | 5.15 |
| 708 | ATLVT012XX2588 | 47522 | TGETGGSINDNH | 4.45 | 1.6 | 1.02 |
| 709 | ATLVT012XX2185 | 47119 | SSTAGAATNDNA | 2.43 | 2.38 | 3.47 |
| 710 | ATLVT012XX1660 | 46594 | SGTSGASSNDNA | 3.09 | 0.69 | 4.5 |
| 711 | ATLVT012XX3374 | 48308 | TSTSAGAINDNH | 0.26 | 5.28 | 3.45 |
| 712 | ATLVT012XX1290 | 46224 | SGATGGSSNDNT | 3.71 | 0.98 | 3.06 |
| 713 | ATLVT012XX375 | 45309 | NGQSGGASNDNT | 2.43 | 3.76 | 1.65 |
| 714 | ATLVT012XX656 | 45590 | NSASGASSNDNH | 3.92 | 3.02 | 0.04 |
| 715 | ATLVT012XX1504 | 46438 | SGQSAGSSNDNA | 4.11 | 2.3 | 0.64 |
| 716 | ATLVT012XX1925 | 46859 | SSESAASSNDNH | 5.37 | 0.28 | 1.06 |
| 717 | ATLVT012XX2792 | 47726 | TGTSAASINDNH | 2.71 | 4.99 | −0.44 |
| 718 | ATLVT012XX2637 | 47571 | TGQAGGSINDNT | 3.06 | 0.9 | 4.2 |
| 719 | ATLVT012XX3256 | 48190 | TSQSGGSSNDNA | 2.31 | 2.59 | 3.29 |
| 720 | ATLVT012XX2235 | 47169 | SSTSGAATNDNT | 2.6 | 1.3 | 4.46 |
| 721 | ATLVT012XX459 | 45393 | NGTAGAASNDNT | 4.46 | 0.98 | 1.67 |
| 722 | ATLVT012XX1460 | 46394 | SGQAAGSTNDNH | 2.01 | 3.04 | 3.23 |
| 723 | ATLVT012XX1604 | 46538 | SGTAAGSINDNH | 2.67 | 3.28 | 1.75 |
| 724 | ATLVT012XX292 | 45226 | NGQAAAATNDNA | 5.64 | −2.38 | 3.93 |
| 725 | ATLVT012XX1170 | 46104 | SGAAAGSSNDNT | 4.28 | 0.97 | 1.95 |
| 726 | ATLVT012XX3346 | 48280 | TSTAGGASNDNA | 0.99 | 3.37 | 4.49 |
| 727 | ATLVT012XX1220 | 46154 | SGASAGSTNDNH | 3.62 | 1.55 | 2.31 |
| 728 | ATLVT012XX1739 | 46673 | SSAAAGASNDNH | 0.03 | 7.11 | 1.32 |
| 729 | ATLVT012XX2031 | 46965 | SSQAAGAINDNT | 3.48 | 1.86 | 2.13 |
| 730 | ATLVT012XX3203 | 48137 | TSQAGGASNDNH | 3.38 | 1.52 | 2.73 |
| 731 | ATLVT012XX2963 | 47897 | TSASGGASNDNH | 1.74 | 4 | 2.37 |
| 732 | ATLVT012XX2187 | 47121 | SSTAGAATNDNT | 1.53 | 2.86 | 4.17 |
| 733 | ATLVT012XX608 | 45542 | NSAAGASSNDNH | 0.17 | 7.35 | 0.7 |
| 734 | ATLVT012XX3385 | 48319 | TSTSGAATNDNA | −0.46 | 4.36 | 5.62 |
| 735 | ATLVT012XX1185 | 46119 | SGAAGASINDNT | 1.68 | 2.83 | 3.89 |
| 736 | ATLVT012XX87 | 45021 | NGASGGASNDNT | 4.09 | 0.61 | 2.6 |
| 737 | ATLVT012XX3163 | 48097 | TSETGGSTNDNA | 5.44 | 1.51 | −0.88 |
| 738 | ATLVT012XX2770 | 47704 | TGTAGGASNDNA | 0.68 | 3.59 | 4.58 |
| 739 | ATLVT012XX1040 | 45974 | NSTAGASSNDNH | 1.01 | 3.13 | 4.61 |
| 740 | ATLVT012XX3283 | 48217 | TSQTAGSINDNA | 4.39 | 0.77 | 1.85 |
| 741 | ATLVT012XX1818 | 46752 | SSASGGSSNDNT | 3.1 | 2.54 | 1.77 |
| 742 | ATLVT012XX2151 | 47085 | SSQTGGATNDNT | 1.25 | 1.23 | 6.61 |
| 743 | ATLVT012XX2058 | 46992 | SSQAGGSSNDNT | 1.63 | 1.54 | 5.57 |
| 744 | ATLVT012XX1036 | 45970 | NSTAGAATNDNA | 1.93 | 2.01 | 4.45 |
| 745 | ATLVT012XX2327 | 47261 | TGAAGAASNDNH | 1.82 | 2.83 | 3.55 |
| 746 | ATLVT012XX92 | 45026 | NGASGGSSNDNH | 2.63 | 1.6 | 3.75 |
| 747 | ATLVT012XX1127 | 46061 | NSTTGAASNDNH | 2.51 | 2.37 | 2.94 |
| 748 | ATLVT012XX29 | 44963 | NGAAGAATNDNH | 1.44 | 3.37 | 3.48 |
| 749 | ATLVT012XX1959 | 46893 | SSESGGATNDNT | 3.39 | 2.26 | 1.53 |
| 750 | ATLVT012XX379 | 45313 | NGQSGGSSNDNA | 1.96 | 3.7 | 2.14 |
| 751 | ATLVT012XX579 | 45513 | NSAAAAASNDNT | 6.7 | −2.03 | 1.37 |
| 752 | ATLVT012XX2914 | 47848 | TSAAGGASNDNA | 1.5 | 2.72 | 4.15 |
| 753 | ATLVT012XX503 | 45437 | NGTSAGSTNDNH | 2.58 | 2.44 | 2.67 |
| 754 | ATLVT012XX2964 | 47898 | TSASGGASNDNT | 3.44 | 1.48 | 2.4 |
| 755 | ATLVT012XX2802 | 47736 | TGTSAGSSNDNT | 2.28 | 2.99 | 2.47 |
| 756 | ATLVT012XX1454 | 46388 | SGQAAGATNDNH | 1.68 | 3.31 | 3.06 |
| 757 | ATLVT012XX1682 | 46616 | SGTTAAATNDNH | 4.49 | 2.63 | −0.89 |
| 758 | ATLVT012XX2773 | 47707 | TGTAGGATNDNA | 1.58 | 0.84 | 6.41 |
| 759 | ATLVT012XX2192 | 47126 | SSTAGASTNDNH | 2.24 | 1.88 | 3.92 |
| 760 | ATLVT012XX1472 | 46406 | SGQAGASTNDNH | 0.75 | 4.49 | 3.12 |
| 761 | ATLVT012XX144 | 45078 | NGATGGSINDNT | 1.86 | 2.63 | 3.59 |
| 762 | ATLVT012XX1136 | 46070 | NSTTGASTNDNH | 1.27 | 3.09 | 4.01 |
| 763 | ATLVT012XX305 | 45239 | NGQAAGATNDNH | 1.39 | 4.64 | 1.81 |
| 764 | ATLVT012XX2221 | 47155 | SSTSAGAINDNA | 3.07 | 1.88 | 2.47 |
| 765 | ATLVT012XX2675 | 47609 | TGQSGGASNDNH | 2.13 | 3.03 | 2.58 |
| 766 | ATLVT012XX1333 | 46267 | SGEAGGATNDNA | 4.29 | 0.52 | 2.1 |
| 767 | ATLVT012XX431 | 45365 | NGQTGGSINDNH | 1.34 | 5.33 | 0.97 |
| 768 | ATLVT012XX1505 | 46439 | SGQSAGSSNDNH | 4.4 | 2 | −0.01 |
| 769 | ATLVT012XX2965 | 47899 | TSASGGATNDNA | 3.61 | 0.82 | 2.86 |
| 770 | ATLVT012XX1128 | 46062 | NSTTGAASNDNT | 2.08 | 2.32 | 3.53 |
| 771 | ATLVT012XX3188 | 48122 | TSQAAGSTNDNH | 1.71 | 2.88 | 3.42 |
| 772 | ATLVT012XX2229 | 47163 | SSTSAGSTNDNT | 2.69 | 19 | 3.01 |
| 773 | ATLVT012XX2724 | 47658 | TGQTGGASNDNT | 1.79 | 1.64 | 4.88 |
| 774 | ATLVT012XX2232 | 47166 | SSTSGAASNDNT | 2.71 | 1.78 | 3.12 |
| 775 | ATLVT012XX1709 | 46643 | SGTTGASSNDNH | 3.62 | 1.21 | 2.27 |
| 776 | ATLVT012XX1247 | 46181 | SGATAAASNDNH | 6.15 | 0.18 | −0.75 |
| 777 | ATLVT012XX1466 | 46400 | SGQAGAATNDNH | 3.29 | 2.08 | 1.68 |
| 778 | ATLVT012XX432 | 45366 | NGQTGGSINDNT | 1.82 | 4.29 | 1.36 |
| 779 | ATLVT012XX2836 | 47770 | TGTTAASSNDNA | 0.77 | 5.55 | 1.54 |
| 780 | ATLVT012XX1217 | 46151 | SGASAGSSNDNH | 4.38 | 1.24 | 0.88 |
| 781 | ATLVT012XX1378 | 46312 | SGESGGASNDNA | 3.38 | 1.26 | 2.57 |
| 782 | ATLVT012XX167 | 45101 | NGEAAGSTNDNH | 2.4 | 0.76 | 4.87 |
| 783 | ATLVT012XX531 | 45465 | NGTTAAASNDNT | 3.38 | −0.03 | 4.19 |
| 784 | ATLVT012XX1292 | 46226 | SGATGGSTNDNH | 3.15 | 1.79 | 2.24 |
| 785 | ATLVT012XX1109 | 46043 | NSTTAASSNDNH | 1.53 | 2.31 | 4.34 |
| 786 | ATLVT012XX2374 | 47308 | TGASGAASNDNA | 3.24 | 1.67 | 2.22 |
| 787 | ATLVT012XX2341 | 47275 | TGAAGGATNDNA | 1.57 | 2.72 | 3.72 |
| 788 | ATLVT012XX382 | 45316 | NGQSGGSINDNA | 2.29 | 2.73 | 2.46 |
| 789 | ATLVT012XX1664 | 46598 | SGTSGASINDNH | 3.01 | 1.42 | 2.9 |
| 790 | ATLVT012XX1371 | 46305 | SGESGAATNDNT | 0.86 | 1.6 | 6.32 |
| 791 | ATLVT012XX3261 | 48195 | TSQSGGSINDNT | 2.14 | 1.87 | 3.77 |
| 792 | ATLVT012XX641 | 45575 | NSASAGAINDNH | 1.43 | 5.31 | 0.56 |
| 793 | ATLVT012XX2435 | 47369 | TGATGGASNDNH | 0.86 | 1.97 | 5.82 |
| 794 | ATLVT012XX1557 | 46491 | SGQTAGSTNDNT | 1.71 | 3.22 | 2.76 |
| 795 | ATLVT012XX607 | 45541 | NSAAGASSNDNA | 3.63 | 2.31 | 0.61 |
| 796 | ATLVT012XX3252 | 48186 | TSQSGGASNDNT | 2.28 | 1.45 | 4.02 |
| 797 | ATLVT012XX1863 | 46797 | SSATGGATNDNT | 2.37 | 2.23 | 2.87 |
| 798 | ATLVT012XX1065 | 45999 | NSTSAASSNDNI | −0.05 | 3.92 | 4.83 |
| 799 | ATLVT012XX458 | 45392 | NGTAGAASNDNH | 3.06 | 1.79 | 2.24 |
| 800 | ATLVT012XX501 | 45435 | NGTSAGSSNDNT | 1.22 | 3.21 | 3.57 |
| 801 | ATLVT012XX362 | 45296 | NGQSGAASNDNH | 5.63 | −0.02 | 0.14 |
| 802 | ATLVT012XX1111 | 46045 | NSTTAASINDNA | −0.51 | 1.18 | 9.12 |
| 803 | ATLVT012XX2891 | 47825 | TSAAAGASNDNH | 1.08 | 4.67 | 1.9 |
| 804 | ATLVT012XX3158 | 48092 | TSETGGAINDNH | 1.14 | 4.27 | 2.29 |
| 805 | ATLVT012XX1106 | 46040 | NSTTAAATNDNH | 0.86 | 6.69 | −0.37 |
| 806 | ATLVT012XX3182 | 48116 | TSQAAGATNDNH | 1.54 | 2.66 | 3.65 |
| 807 | ATLVT012XX3422 | 48356 | TSTTAGATNDNH | 2.38 | 3.2 | 1.5 |
| 808 | ATLVT012XX907 | 45841 | NSQAGGSSNDNA | 2.12 | 3.63 | 1.39 |
| 809 | ATLVT012XX1753 | 46687 | SSAAGAATNDNA | 2.11 | 2.32 | 3.09 |
| 810 | ATLVT012XX2207 | 47141 | SSTSAAASNDNH | 0.62 | 4.32 | 3.06 |
| 811 | ATLVT012XX2002 | 46936 | SSETGGASNDNA | 1.25 | 2.03 | 4.91 |
| 812 | ATLVT012XX38 | 44972 | NGAAGGASNDNH | 1.7 | 2.73 | 3.22 |
| 813 | ATLVT012XX1771 | 46705 | SSAAGGSINDNA | 2.2 | 3.25 | 1.68 |
| 814 | ATLVT012XX1018 | 45952 | NSTAAASTNDNA | 1.72 | 3.27 | 2.47 |
| 815 | ATLVT012XX2801 | 47735 | TGTSAGSSNDNH | 3.86 | 2.29 | 0.02 |
| 816 | ATLVT012XX1707 | 46641 | SGTTGAATNDNT | 3.97 | 0.24 | 2.46 |
| 817 | ATLVT012XX1566 | 46500 | SGQTGASSNDNT | 0.54 | 3.71 | 3.88 |
| 818 | ATLVT012XX3110 | 48044 | TSESGGATNDNH | 2.01 | 1.02 | 4.81 |
| 819 | ATLVT012XX1021 | 45955 | NSTAAGASNDNA | 2.08 | 3.31 | 1.74 |
| 820 | ATLVT012XX41 | 44975 | NGAAGGATNDNH | 2.3 | 2.19 | 2.81 |
| 821 | ATLVT012XX1057 | 45991 | NSTSAAASNDNA | 4.15 | 1.26 | 0.81 |
| 822 | ATLVT012XX1652 | 46586 | SGTSAGSTNDNH | 3.41 | 0.93 | 2.51 |
| 823 | ATLVT012XX1167 | 46101 | SGAAAGAINDNT | 2.46 | 2 | 2.76 |
| 824 | ATLVT012XX2011 | 46945 | SSETGGSINDNA | 4.04 | 1.06 | 1.24 |
| 825 | ATLVT012XX2091 | 47025 | SSQSGAATNDNT | 3.06 | 0.77 | 3.3 |
| 826 | ATLVT012XX2388 | 47322 | TGASGGASNDNT | 1.18 | 3.18 | 3.41 |
| 827 | ATLVT012XX714 | 45648 | NSATGGATNDNT | 0.28 | 2.7 | 5.55 |
| 828 | ATLVT012XX3107 | 48041 | TSESGGASNDNH | 3.5 | 1.58 | 1.47 |
| 829 | ATLVT012XX1768 | 46702 | SSAAGGSSNDNA | 3.16 | 1.65 | 1.93 |
| 830 | ATLVT012XX859 | 45793 | NSETGGSSNDNA | 3.73 | 1.78 | 0.78 |
| 831 | ATLVT012XX2579 | 47513 | TGETGGASNDNH | 2.92 | 3 | 0.58 |
| 832 | ATLVT012XX617 | 45551 | NSAAGGATNDNH | 1.9 | 3.02 | 2.29 |
| 833 | ATLVT012XX2328 | 47262 | TGAAGAASNDNT | 1.37 | 2.75 | 3.54 |
| 834 | ATLVT012XX140 | 45074 | NGATGGSSNDNH | 2.74 | 0.47 | 4.1 |
| 835 | ATLVT012XX138 | 45072 | NGATGGATNDNT | 0.74 | 5.25 | 1.38 |
| 836 | ATLVT012XX86 | 45020 | NGASGGASNDNH | 3.37 | 1.33 | 1.87 |
| 837 | ATLVT012XX1165 | 46099 | SGAAAGAINDNA | 1.7 | 2.63 | 3.05 |
| 838 | ATLVT012XX1864 | 46798 | SSATGGSSNDNA | 4.29 | 0.7 | 1.1 |
| 839 | ATLVT012XX1562 | 46496 | SGQTGAATNDNH | 1.58 | 2.55 | 3.37 |
| 840 | ATLVT012XX1455 | 46389 | SGQAAGATNDNT | 1.96 | 2.42 | 2.87 |
| 841 | ATLVT012XX3296 | 48230 | TSQTGASTNDNH | 0.43 | 6.62 | 0.08 |
| 842 | ATLVT012XX1815 | 46749 | SSASGGATNDNT | 1.54 | 3.94 | 1.63 |
| 843 | ATLVT012XX2279 | 47213 | SSTTGAASNDNH | 2.72 | 2.62 | 1.26 |
| 844 | ATLVT012XX1695 | 46629 | SGTTAGAINDNT | 1.07 | 3.29 | 3.21 |
| 845 | ATLVT012XX638 | 45572 | NSASAGASNDNH | 3.9 | 2.5 | −0.64 |
| 846 | ATLVT012XX1181 | 46115 | SGAAGASSNDNH | 1.7 | 3.06 | 2.4 |
| 847 | ATLVT012XX1809 | 46743 | SSASGASINDNT | 2.41 | 0.5 | 4.47 |
| 848 | ATLVT012XX1330 | 46264 | SGEAGGASNDNA | 3.23 | 1.39 | 1.9 |
| 849 | ATLVT012XX2344 | 47278 | TGAAGGSSNDNA | 1.03 | 2.78 | 3.88 |
| 850 | ATLVT012XX2781 | 47715 | TGTAGGSINDNT | 0.99 | 1.61 | 5.44 |
| 851 | ATLVT012XX2858 | 47792 | TGTTGAATNDNH | 2.58 | 1.93 | 2.29 |
| 852 | ATLVT012XX1549 | 46483 | SGQTAGAINDNA | 1 | 5.87 | −0.06 |
| 853 | ATLVT012XX3380 | 48314 | TSTSAGSTNDNH | 0.5 | 2.69 | 4.88 |
| 854 | ATLVT012XX2436 | 47370 | TGATGGASNDNT | 0.38 | 1.16 | 7.04 |
| 855 | ATLVT012XX1498 | 46432 | SGQSAGASNDNA | 1.9 | 4.14 | 0.61 |
| 856 | ATLVT012XX452 | 45386 | NGTAAGSSNDNH | 4.28 | 1.44 | 0 |
| 857 | ATLVT012XX999 | 45933 | NSQTGGASNDNT | 4.32 | 0.62 | 0.98 |
| 858 | ATLVT012XX2028 | 46962 | SSQAAGASNDNT | 1.95 | 2.37 | 2.79 |
| 859 | ATLVT012XX1335 | 46269 | SGEAGGATNDNT | 1.48 | 3.16 | 2.55 |
| 860 | ATLVT012XX2789 | 47723 | TGTSAASSNDNH | 3 | 1.9 | 1.54 |
| 861 | ATLVT012XX1126 | 46060 | NSTTGAASNDNA | 0.27 | 4.11 | 3.36 |
| 862 | ATLVT012XX2920 | 47854 | TSAAGGSSNDNA | 1.23 | 3.23 | 2.86 |
| 863 | ATLVT012XX3297 | 48231 | TSQTGASTNDNT | 3.27 | 1.79 | 1.19 |
| 864 | ATLVT012XX3278 | 48212 | ISQTAGAINDNH | 2.46 | 2.01 | 2.3 |
| 865 | ATLVT012XX1642 | 46576 | SGTSAGASNDNA | 1.94 | 2.32 | 2.79 |
| 866 | ATLVT012XX449 | 45383 | NGTAAGAINDNH | 2.29 | 2.54 | 1.91 |
| 867 | ATLVT012XX339 | 45273 | NGQSAAASNDNT | 0.84 | 6.08 | −0.18 |
| 868 | ATLVT012XX3416 | 48350 | TSTTAASINDNH | 0.57 | 5.21 | 1.38 |
| 869 | ATLVT012XX2722 | 47656 | TGQTGGASNDNA | 1.51 | 2.25 | 3.56 |
| 870 | ATLVT012XX637 | 45571 | NSASAGASNDNA | 3.05 | 2.77 | 0.26 |
| 871 | ATLVT012XX3335 | 48269 | TSTAGAASNDNH | 4.14 | 0.6 | 1.17 |
| 872 | ATLVT012XX2064 | 46998 | SSQSAAASNDNT | 2.39 | −1.01 | 6.24 |
| 873 | ATLVT012XX1506 | 46440 | SGQSAGSSNDNT | 1.55 | 4.67 | 0.34 |
| 874 | ATLVT012XX2137 | 47071 | SSQTGAATNDNA | 2.94 | −0.47 | 4.58 |
| 875 | ATLVT012XX2492 | 47426 | TGEAGGSINDNH | 4.67 | 0.46 | 0.4 |
| 876 | ATLVT012XX1543 | 46477 | SGQTAASTNDNA | 2.7 | 2.75 | 0.83 |
| 877 | ATLVT012XX1744 | 46678 | SSAAAGSSNDNA | 3.59 | 1.28 | 1.19 |
| 878 | ATLVT012XX85 | 45019 | NGASGGASNDNA | 2.59 | 1.8 | 2.22 |
| 879 | ATLVT012XX2336 | 47270 | TGAAGASTNDNH | 1.93 | 2.16 | 2.9 |
| 880 | ATLVT012XX380 | 45314 | NGQSGGSSNDNH | 1.67 | 3.58 | 1.51 |
| 881 | ATLVT012XX424 | 45358 | NGQTGGATNDNA | 2.96 | 2.79 | 0.31 |
| 882 | ATLVT012XX2081 | 47015 | SSQSAGSSNDNH | 1.4 | 4.6 | 0.63 |
| 883 | ATLVT012XX1064 | 45998 | NSTSAASSNDNH | −0.03 | 2.88 | 5.3 |
| 884 | ATLVT012XX1603 | 46537 | SGTAAGSTNDNA | 3.27 | 1.65 | 1.23 |
| 885 | ATLVT012XX1511 | 46445 | SGQSGAASNDNH | 3.47 | 1.41 | 1.19 |
| 886 | ATLVT012XX1805 | 46739 | SSASGASSNDNH | 1.48 | 4.08 | 1.17 |
| 887 | ATLVT012XX1711 | 46645 | SGTTGASTNDNA | 3.94 | 0.6 | 1.42 |
| 888 | ATLVT012XX2386 | 47320 | TGASGGASNDNA | 2.13 | 2.33 | 2.3 |
| 889 | ATLVT012XX2012 | 46946 | SSETGGSINDNH | 3.67 | 1.37 | 0.89 |
| 890 | ATLVT012XX129 | 45063 | NGATGASSNDNT | 0.56 | 2.7 | 4.5 |
| 891 | ATLVT01XXX142 | 45076 | NGATGGSINDNA | 2.85 | 1 | 2.76 |
| 892 | ATLVT012XX1169 | 46103 | SGAAAGSSNDNH | 1.97 | 2.66 | 2.15 |
| 893 | ATLVT012XX457 | 45391 | NGTAGAASNDNA | 4.73 | −0.32 | 1.23 |
| 894 | ATLVT012XX1650 | 46584 | SGTSAGSSNDNT | 1.29 | 3.16 | 2.65 |
| 895 | ATLVT012XX1113 | 46047 | NSTTAASINDNT | 0.02 | 1.07 | 7.49 |
| 896 | ATLVT012XX3351 | 48285 | TSTAGGATNDNT | 0.93 | 1.41 | 5.46 |
| 897 | ATLVT012XX2218 | 47152 | SSTSAGASNDNA | 2.75 | 1 | 2.86 |
| 898 | ATLVT012XX3257 | 48191 | TSQSGGSSNDNH | 2.05 | 2.36 | 2.3 |
| 899 | ATLVT012XX666 | 45600 | NSASGGATNDNT | 1.1 | 1.69 | 4.81 |
| 900 | ATLVT012XX1869 | 46803 | SSATGGSINDNT | 2.9 | 1.48 | 1.96 |
| 901 | ATLVT012XX1178 | 46112 | SGAAGAATNDNH | 1.52 | 2.43 | 3.06 |
| 902 | ATLVT012XX93 | 45027 | NGASGGSSNDNT | 0.13 | 1.22 | 6.98 |
| 903 | ATLVT012XX2911 | 47845 | TSAAGASINDNA | 2.71 | 1.99 | 1.56 |
| 904 | ATLVT012XX311 | 45245 | NGQAAGSTNDNH | 2.44 | 1.97 | 2.04 |
| 905 | ATLVT012XX3014 | 47948 | TSATGGAINDNH | 2.65 | 3.39 | −0.14 |
| 906 | ATLVT012XX887 | 45821 | NSQAAGSTNDNH | 1.44 | 3.11 | 2.29 |
| 907 | ATLVT012XX2612 | 47546 | TGQAAGSINDNH | 0.69 | 1.26 | 5.93 |
| 908 | ATLVT012XX125 | 45059 | NGATGAATNDNH | 1.66 | 4.39 | 0.23 |
| 909 | ATLVT012XX1112 | 46046 | NSTTAASINDNH | −0.1 | 3.22 | 4.75 |
| 910 | ATLVT012XX3367 | 48301 | TSTSAASTNDNA | 2.02 | 0.26 | 4.9 |
| 911 | ATLVT012XX381 | 45315 | NGQSGGSSNDNT | −0.19 | 5.4 | 2.09 |
| 912 | ATLVT012XX3387 | 48321 | TSTSGAATNDNT | 0.67 | 2.74 | 4.03 |
| 913 | ATLVT012XX2708 | 47642 | TGQTAGSINDNH | 1.8 | 3 | 1.76 |
| 914 | ATLVT012XX1223 | 46157 | SGASGAASNDNH | 3.18 | 1.5 | 1.31 |
| 915 | ATLVT012XX454 | 45388 | NGTAAGSINDNA | 3.53 | 0.83 | 1.56 |
| 916 | ATLVT012XX3019 | 47953 | TSATGGSINDNA | 1.76 | 4.92 | −0.68 |
| 917 | ATLVT012XX2681 | 47615 | TGQSGGSSNDNH | 2.26 | 2.65 | 1.37 |
| 918 | ATLVT012XX94 | 45028 | NGASGGSINDNA | 1.91 | 1.8 | 3.05 |
| 919 | ATLVT012XX2193 | 47127 | SSTAGASTNDNT | 1.73 | 0.12 | 5.52 |
| 920 | ATLVT012XX1992 | 46926 | SSETGAASNDNT | 5.65 | −0.64 | −0.24 |
| 921 | ATLVT012XX1763 | 46697 | SSAAGGASNDNH | 1.6 | 2.8 | 2.29 |
| 922 | ATLVT012XX1678 | 46612 | SGTTAAASNDNA | 2.61 | 4.5 | −1.64 |
| 923 | ATLVT012XX3116 | 48050 | TSESGGSINDNH | 1.74 | 3.01 | 1.76 |
| 924 | ATLVT012XX2903 | 47837 | TSAAGAASNDNH | 1.47 | 2.32 | 3.1 |
| 925 | ATLVT012XX2223 | 47157 | SSTSAGATNDNT | 2.17 | 1.35 | 3.15 |
| 926 | ATLVT012XX3068 | 48002 | TSEAGGSTNDNH | 3.41 | 0.72 | 1.83 |
| 927 | ATLVT012XX3178 | 48112 | TSQAAGASNDNA | 1.16 | 2.54 | 3.34 |
| 928 | ATLVT012XX2946 | 47880 | TSASAGSSNDNT | 4.37 | 0.93 | −0.09 |
| 929 | ATLVT012XX2349 | 47283 | TGAAGGSTNDNT | 1.23 | 2.28 | 3.55 |
| 930 | ATLVT012XX1954 | 46888 | SSESGGASNDNA | 2.68 | 2.55 | 0.71 |
| 931 | ATLVT012XX2972 | 47906 | TSASGGSINDNH | 3.67 | 0.76 | 1.31 |
| 932 | ATLVT012XX135 | 45069 | NGATGGASNDNT | 1.49 | 2.67 | 2.58 |
| 933 | ATLVT012XX3389 | 48323 | ISTSGASSNDNH | −0.57 | 6.16 | 1.63 |
| 934 | ATLVT012XX1253 | 46187 | SGATAASSNDNH | 3.76 | 1.51 | 0.18 |
| 935 | ATLVT012XX1221 | 46155 | SGASAGSTNDNT | 0.9 | 0.2 | 6.76 |
| 936 | ATLVT012XX1632 | 46566 | SGTSAAASNDNT | 1.94 | −0.27 | 5.58 |
| 937 | ATLVT012XX507 | 45441 | NGTSGAASNDNT | 1.67 | 3.99 | 0.56 |
| 938 | ATLVT012XX3382 | 48316 | TSTSGAASNDNA | 1.01 | 2.5 | 3.59 |
| 939 | ATLVT012XX3248 | 48182 | TSQSGASTNDNH | 1.55 | 3.7 | 1.11 |
| 940 | ATLVT012XX533 | 45467 | NGTTAAATNDNH | 2.67 | 2.9 | 0.19 |
| 941 | ATLVT012XX2633 | 47567 | TGQAGGSSNDNH | 4.32 | 0.46 | 0.51 |
| 942 | ATLVT012XX3388 | 48322 | TSTSGASSNDNA | 1.2 | 3.61 | 1.8 |
| 943 | ATLVT012XX1908 | 46842 | SSEAGGASNDNT | 2.05 | 2.76 | 1.42 |
| 944 | ATLVT012XX712 | 45646 | NSATGGATNDNA | 1.23 | 1.74 | 4.12 |
| 945 | ATLVT012XX2807 | 47741 | TGTSGAASNDNH | 2.51 | 1.68 | 2 |
| 946 | ATLVT012XX1957 | 46891 | SSESGGATNDNA | 3.43 | 1.58 | 0.51 |
| 947 | ATLVT012XX1865 | 46799 | SSATGGSSNDNH | 4.31 | 1.12 | −0.4 |
| 948 | ATLVT012XX329 | 45263 | NGQAGGATNDNH | 2.56 | 1.66 | 1.89 |
| 949 | ATLVT012XX610 | 45544 | NSAAGASINDNA | 1.49 | 2.61 | 2.5 |
| 950 | ATLVT012XX1587 | 46521 | SGTAAAAINDNT | 2.35 | 1.7 | 2.19 |
| 951 | ATLVT012XX1163 | 46097 | SGAAAGASNDNH | 1.62 | 2.2 | 2.8 |
| 952 | ATLVT012XX1369 | 46303 | SGESGAATNDNA | 2.19 | 1.47 | 2.73 |
| 953 | ATLVT012XX2907 | 47841 | TSAAGAATNDNT | 1.49 | 2.38 | 2.76 |
| 954 | ATLVT012XX1703 | 46637 | SGTTGAASNDNH | 3.67 | 0.66 | 1.21 |
| 955 | ATLVT012XX673 | 45607 | NSATAAASNDNA | 4.46 | −0.79 | 1.71 |
| 956 | ATLVT012XX2783 | 47717 | TGTSAAASNDNH | 1.13 | 0.03 | 6.38 |
| 957 | ATLVT012XX510 | 45444 | NGTSGAATNDNT | 1.98 | 0.69 | 4.06 |
| 958 | ATLVT012XX3002 | 47936 | TSATGAATNDNH | 3.82 | 0.97 | 0.53 |
| 959 | ATLVT012XX2603 | 47537 | TGQAAGASNDNH | 1.16 | 2.17 | 3.54 |
| 960 | ATLVT012XX611 | 45545 | NSAAGASTNDNH | 0.65 | 4.08 | 1.94 |
| 961 | ATLVT012XX1697 | 46631 | SGTTAGSSNDNH | 0.38 | 5.08 | 1.11 |
| 962 | ATLVT012XX613 | 45547 | NSAAGGASNDNA | 0.83 | 3.31 | 2.6 |
| 963 | ATLVT012XX88 | 45022 | NGASGGATNDNA | 1.5 | 0.49 | 5.07 |
| 964 | ATLVT012XX2093 | 47027 | SSQSGASSNDNH | 1.6 | 1.02 | 4.22 |
| 965 | ATLVT012XX603 | 45537 | NSAAGAASNDNT | 2.49 | 1.5 | 2.08 |
| 966 | ATLVT012XX766 | 45700 | NSEAGGSTNDNA | 1.13 | 4.38 | 0.69 |
| 967 | ATLVT012XX530 | 45464 | NGTTAAASNDNH | 2.22 | 3.19 | 0.36 |
| 968 | ATLVT012XX2420 | 47354 | TGATAGSINDNH | 2.64 | 0.61 | 2.95 |
| 969 | ATLVT012XX1121 | 46055 | NSTTAGSSNDNH | 2.71 | 1.96 | 1.09 |
| 970 | ATLVT012XX2078 | 47012 | SSQSAGAINDNH | 1.69 | 3.11 | 1.35 |
| 971 | ATLVT012XX374 | 45308 | NGQSGGASNDNH | 1.46 | 1.83 | 3.4 |
| 972 | ATLVT012XX1512 | 46446 | SGQSGAASNDNT | 3.83 | 0.58 | 0.91 |
| 973 | ATLVT012XX1507 | 46441 | SGQSAGSINDNA | 1.94 | 3.39 | 0.53 |
| 974 | ATLVT012XX1937 | 46871 | SSESAGSSNDNH | −1.02 | 4.02 | 4.77 |
| 975 | ATLVT012XX3383 | 48317 | TSTSGAASNDNH | 1.02 | 3.02 | 2.57 |
| 976 | ATLVT012XX2321 | 47255 | TGAAAGSSNDNH | 1.55 | 2.31 | 2.57 |
| 977 | ATLVT012XX2440 | 47374 | TGATGGSSNDNA | 0.39 | 4.19 | 2.12 |
| 978 | ATLVT012XX3381 | 48315 | TSTSAGSINDNT | −0.35 | 1.4 | 6.98 |
| 979 | ATLVT012XX3164 | 48098 | TSETGGSINDNH | 2.86 | 2.98 | −0.56 |
| 980 | ATLVT012XX1943 | 46877 | SSESGAASNDNH | 1.42 | 1.22 | 4.13 |
| 981 | ATLVT012XX1293 | 46227 | SGATGGSINDNT | 1.93 | 1.49 | 2.92 |
| 982 | ATLVT012XX1282 | 46216 | SGATGGASNDNA | 3.62 | 0.64 | 1.11 |
| 983 | ATLVT012XX216 | 45150 | NGESAGSTNDNT | 2.91 | 2.05 | 0.5 |
| 984 | ATLVT012XX624 | 45558 | NSAAGGSINDNT | 0.54 | 2.84 | 3.55 |
| 985 | ATLVT012XX1271 | 46205 | SGATGAASNDNH | 1.48 | 4.64 | −0.39 |
| 986 | ATLVT012XX2441 | 47375 | TGATGGSSNDNH | 3.27 | 0.52 | 1.83 |
| 987 | ATLVT012XX1611 | 46545 | SGTAGAATNDNI | −0.05 | 1.41 | 6.38 |
| 988 | ATLVT012XX512 | 45446 | NGTSGASSNDNH | 1.01 | 1.87 | 3.97 |
| 989 | ATLVT012XX2660 | 47594 | TGQSAGSINDNH | 1.94 | 3.59 | 0.15 |
| 990 | ATLVT012XX3349 | 48283 | TSTAGGATNDNA | 0.68 | 1.72 | 4.73 |
| 991 | ATLVT012XX2335 | 47269 | TGAAGASTNDNA | 0.97 | 2.75 | 2.91 |
| 992 | ATLVT012XX3323 | 48257 | TSTAAGASNDNH | 1.06 | 3.43 | 1.86 |
| 993 | ATLVT012XX1690 | 46624 | SGTTAGASNDNA | −0.09 | 1.54 | 6.26 |
| 994 | ATLVT012XX929 | 45863 | NSQSAGATNDNH | 3.93 | 1.28 | −0.29 |
| 995 | ATLVT012XX376 | 45310 | NGQSGGATNDNA | 3.88 | 0.38 | 0.93 |
| 996 | ATLVT012XX1337 | 46271 | SGEAGGSENDNH | 2.33 | 2.74 | 0.55 |
| 997 | ATLVT012XX904 | 45838 | NSQAGGATNDNA | 3.79 | 1 | 0.27 |
| 998 | ATLVT012XX2036 | 46970 | SSQAAGSINDNH | 1.42 | 2.18 | 2.81 |
| 999 | ATLVT012XX2343 | 47277 | TGAAGGATNDNT | 1.07 | 2.54 | 2.93 |
| 1000 | ATLVT012XX1061 | 45995 | NSTSAAATNDNH | 2.99 | 2.5 | −0.31 |
| 1001 | ATLVT012XX618 | 45552 | NSAAGGATNDNT | 1.41 | 1.82 | 3.27 |
| 1002 | ATLVT012XX881 | 45815 | NSQAAGATNDNH | 1.56 | 2.5 | 2.13 |
| 1003 | ATLVT012XX3238 | 48172 | TSQSGAASNDNA | −0.22 | 1 | 7.1 |
| 1004 | ATLVT012XX3155 | 48089 | TSETGGASNDNH | 2.93 | 2.15 | 0.22 |
| 1005 | ATLVT012XX1961 | 46895 | SSESGGSSNDNH | 3.36 | 1.85 | −0.12 |
| 1006 | ATLVT012XX2892 | 47826 | TSAAAGASNDNT | 1.13 | 2.87 | 2.37 |
| 1007 | ATLVT012XX495 | 45429 | NGTSAGASNDNT | 2.98 | 1.63 | 0.8 |
| 1008 | ATLVT012XX3242 | 48176 | TSQSGAATNDNH | 1.96 | 2.11 | 1.92 |
| 1009 | ATLVT012XX1341 | 46275 | SGEAGGSINDNT | 1.72 | 3.02 | 1.17 |
| 1010 | ATLVT012XX764 | 45698 | NSEAGGSSNDNH | 1.96 | 0.83 | 3.56 |
| 1011 | ATLVT012XX3290 | 48224 | TSQTGAATNDNH | 1.16 | 2.05 | 3.34 |
| 1012 | ATLVT012XX2618 | 47552 | TGQAGAATNDNH | 1.94 | 1.44 | 2.75 |
| 1013 | ATLVT012XX90 | 45024 | NGASGGATNDNT | 2.08 | 1.43 | 2.51 |
| 1014 | ATLVT012XX2088 | 47022 | SSQSGAASNDNT | 2.71 | 0.89 | 2.13 |
| 1015 | ATLVT012XX74 | 45008 | NGASGAASNDNH | 2.14 | 1.69 | 2.08 |
| 1016 | ATLVT012XX1386 | 46320 | SGESGGSSNDNT | 1.87 | 1.43 | 2.86 |
| 1017 | ATLVT012XX3205 | 48139 | TSQAGGATNDNA | 2.31 | 1.75 | 1.68 |
| 1018 | ATLVT012XX429 | 45363 | NG?TGGSSNDNT | 2 | 2.54 | 1.19 |
| 1019 | ATLVT012XX1713 | 46647 | SGTTGASTNDNI | 3.15 | 0.64 | 1.63 |
| 1020 | ATLVT012XX2379 | 47313 | TGASGAATNDNT | 3.2 | 0.97 | 1.13 |
| 1021 | ATLVT012XX689 | 45623 | NSATAGAINDNH | 1.85 | 1.55 | 2.7 |
| 1022 | ATLVT012XX3393 | 48327 | TSTSGASTNDNT | 0.87 | 2.59 | 3.04 |
| 1023 | ATLVT012XX3180 | 48114 | TSQAAGASNDNT | 1.05 | 2.31 | 3.09 |
| 1024 | ATLVT012XX2359 | 47293 | TGASAASTNDNA | −0.14 | 6.61 | −0.42 |
| 1025 | ATLVT012XX1364 | 46298 | SGESAGSTNDNH | 2.4 | 3.77 | −1.13 |
| 1026 | ATLVT012XX331 | 45265 | NGQAGGSSNDNA | 1.73 | 1.8 | 2.55 |
| 1027 | ATLVT012XX639 | 45573 | NSASAGASNDNT | 2.35 | 2.87 | 0.11 |
| 1028 | ATLVT012XX3338 | 48272 | TSTAGAATNDNH | 2.87 | 1.44 | 1.04 |
| 1029 | ATLVT012XX2069 | 47003 | SSQSAASSNDNH | 0.84 | −0.32 | 6.78 |
| 1030 | ATLVT012XX690 | 45624 | NSATAGATNDNT | −0.14 | 2.71 | 4.56 |
| 1031 | ATLVT012XX83 | 45017 | NGASGASTNDNH | 0.86 | 3.92 | 1.27 |
| 1032 | ATLVT012XX1461 | 46395 | SGQAAGSINDNT | 2.63 | 1.1 | 1.84 |
| 1033 | ATLVT012XX1487 | 46421 | SGQSAAASNDNH | 4.2 | −0.58 | 1.31 |
| 1034 | ATLVT012XX1376 | 46310 | SGESGASTNDNH | −0.06 | 6.41 | −0.39 |
| 1035 | ATLVT012XX77 | 45011 | NGASGAATNDNH | 1.52 | 2.48 | 1.94 |
| 1036 | ATLVT012XX2322 | 47256 | TGAAAGSSNDNT | 1.62 | 1.76 | 2.7 |
| 1037 | ATLVT012XX3217 | 48151 | TSQSAAATNDNA | 1.61 | 3.53 | 0.41 |
| 1038 | ATLVT012XX120 | 45054 | NGATAGSINDNT | 1.96 | 0.28 | 3.99 |
| 1039 | ATLVT012XX649 | 45583 | NSASGAASNDNA | 3.89 | 0.07 | 0.94 |
| 1040 | ATLVT012XX1262 | 46196 | SGATAGAINDNH | 3.94 | 0.34 | 0.51 |
| 1041 | ATLVT012XX2087 | 47021 | SSQSGAASNDNH | 3.23 | 0.65 | 1.31 |
| 1042 | ATLVT012XX614 | 45548 | NSAAGGASNDNH | 1.42 | 2.2 | 2.41 |
| 1043 | ATLVT012XX2230 | 47164 | SSTSGAASNDNA | 2.49 | 1.23 | 1.82 |
| 1044 | ATLVT012XX1409 | 46343 | SGETAGSSNDNH | 0.39 | 5.09 | 0.45 |
| 1045 | ATLVT012XX1563 | 46497 | SGQTGAATNDNT | 0.53 | 0.75 | 5.77 |
| 1046 | ATLVT012XX1850 | 46784 | SSATGAATNDNH | 1.8 | 4.06 | −0.66 |
| 1047 | ATLVT012XX3109 | 48043 | TSESGGATNDNA | 3.56 | 0.45 | 0.95 |
| 1048 | ATLVT012XX1616 | 46550 | SGTAGASTNDNH | 1.8 | 1.96 | 2.02 |
| 1049 | ATLVT012XX3330 | 48264 | TSTAAGSSNDNT | 2.53 | 1.23 | 1.71 |
| 1050 | ATLVT01XXX1663 | 46597 | SGTSGASTNDNA | 2.37 | 0.24 | 3.25 |
| 1051 | ATLVT012XX231 | 45165 | NGESGGASNDNT | 3.86 | 0.82 | −0.06 |
| 1052 | ATLVT012XX1802 | 46736 | SSASGAATNDNH | 1.57 | 3.55 | 0.35 |
| 1053 | ATLVT012XX2183 | 47117 | SSTAGAASNDNH | 1.88 | 1.38 | 2.59 |
| 1054 | ATLVT012XX983 | 45917 | NSQTAGSTNDNH | 4.03 | 0.98 | −0.59 |
| 1055 | ATLVT012XX317 | 45251 | NGQAGAATNDNH | 3.91 | 0.21 | 0.59 |
| 1056 | ATLVT012XX3069 | 48003 | TSEAGGSINDNT | 3.38 | 0.27 | 1.43 |
| 1057 | ATLVT012XX122 | 45056 | NGATGAASNDNH | 3.22 | 1.23 | 0.46 |
| 1058 | ATLVT012XX1823 | 46757 | SSATAAASNDNH | 1.2 | 4.15 | 0.33 |
| 1059 | ATLVT012XX2117 | 47051 | SSQTAASSNDNH | 0.29 | −0.06 | 7.12 |
| 1060 | ATLVT012XX218 | 45152 | NGESGAASNDNH | 5.39 | −1.06 | −0.32 |
| 1061 | ATLVT012XX2727 | 47661 | TGQTGGATNDNT | 1 | 0.42 | 5.3 |
| 1062 | ATLVT012XX2702 | 47636 | TGQTAGATNDNH | 3.6 | −0.33 | 1.78 |
| 1063 | ATLVT012XX455 | 45389 | NGTAAGSINDNH | 2.87 | 0.78 | 1.59 |
| 1064 | ATLVT012XX2849 | 47783 | IGTTAGSSNDNH | 1.57 | 2.71 | 1.35 |
| 1065 | ATLVT012XX1960 | 46894 | SSESGGSSNDNA | 2.62 | 1.76 | 0.77 |
| 1066 | ATLVT012XX695 | 45629 | NSATAGSINDNH | 0.89 | 0.17 | 5.77 |
| 1067 | ATLVT012XX845 | 45779 | NSETGAATNDNH | 3.41 | 0.67 | 0.8 |
| 1068 | ATLVT012XX338 | 45272 | NGQSAAASNDNH | 3.48 | 0.65 | 0.68 |
| 1069 | ATLVT012XX2452 | 47386 | TGEAAASSNDNA | 0.89 | 5.5 | −1.12 |
| 1070 | ATLVT012XX1499 | 46433 | SGQSAGASNDNH | 2.22 | 1.29 | 2.01 |
| 1071 | ATLVT012XX96 | 45030 | NGASGGSINDNT | 1.37 | 0.82 | 4.06 |
| 1072 | ATLVT012XX1308 | 46242 | SGEAAGASNDNT | 1.02 | 4.38 | 0.06 |
| 1073 | ATLVT012XX3020 | 47954 | TSATGGSINDNH | 4.19 | 0.74 | −0.69 |
| 1074 | ATLVT012XX2345 | 47279 | TGAAGGSSNDNH | 1.17 | 1.88 | 3.01 |
| 1075 | ATLVT012XX3017 | 47951 | TSATGGSSNDNH | 4.27 | 0.65 | −0.71 |
| 1076 | ATLVT012XX3384 | 48318 | TSTSGAASNDNT | 1.2 | 2.32 | 2.38 |
| 1077 | ATLVT012XX1180 | 46114 | SGAAGASSNDNA | 1.09 | 1.96 | 3.04 |
| 1078 | ATLVT012XX615 | 45549 | NSAAGGASNDNT | 0.74 | 1.53 | 4.18 |
| 1079 | ATLVT012XX2320 | 47254 | TGAAAGSSNDNA | 1.75 | 1.86 | 2.02 |
| 1080 | ATLVT012XX3436 | 48370 | TSTTGASSNDNA | 3.15 | 1.4 | 0.18 |
| 1081 | ATLVT012XX883 | 45817 | NSQAAGSSNDNA | 1.47 | 2.19 | 2.04 |
| 1082 | ATLVT012XX134 | 45068 | NGATGGASNDNH | 2.18 | 0.79 | 2.62 |
| 1083 | ATLVT012XX237 | 45171 | NGESGGSSNDNT | 2.4 | 2.34 | 0.24 |
| 1084 | ATLVT012XX1000 | 45934 | NSQTGGATNDNA | 2.42 | 1.24 | 1.62 |
| 1085 | ATLVT012XX948 | 45882 | NSQSGASTNDNT | 2.19 | 1.48 | 1.69 |
| 1086 | ATLVT012XX3165 | 48099 | TSETGGSINDNT | 3.9 | 1.03 | −0.65 |
| 1087 | ATLVT012XX1316 | 46250 | SGEAAGSINDNH | 3.02 | 0.86 | 1.07 |
| 1088 | ATLVT012XX2089 | 47023 | SSQSGAATNDNA | 2.1 | 1.31 | 2.04 |
| 1089 | ATLVT012XX2227 | 47161 | SSTSAGSINDNA | 1.83 | 1.62 | 2.11 |
| 1090 | ATLVT012XX3285 | 48219 | TSQTAGSINDNT | 1.73 | 0.73 | 3.4 |
| 1091 | ATLVT012XX1331 | 46265 | SGEAGGASNDNH | 1.21 | 1.21 | 3.66 |
| 1092 | ATLVT012XX1762 | 46696 | SSAAGGASNDNA | 2.32 | 1.51 | 1.36 |
| 1093 | ATLVT012XX1232 | 46166 | SGASGASTNDNH | 1.97 | 2.2 | 1.06 |
| 1094 | ATLVT012XX2048 | 46982 | SSQAGASTNDNH | 2.41 | 1.92 | 0.67 |
| 1095 | ATLVT012XX286 | 45220 | NGETGGSINDNA | −0.34 | 4.88 | 1.57 |
| 1096 | ATLVT012XX3011 | 47945 | TSATGGASNDNH | 2.02 | 2.13 | 1.06 |
| 1097 | ATLVT012XX2683 | 47617 | TGQSGGSINDNA | −0.39 | 4.47 | 2.17 |
| 1098 | ATLVT012XX1182 | 46116 | SGAAGASSNDNT | 1.01 | 2.06 | 2.88 |
| 1099 | ATLVT012XX504 | 45438 | NGTSAGSINDNT | 1.56 | 1.07 | 3.2 |
| 1100 | ATLVT012XX3392 | 48326 | TSTSGASTNDNH | 1.1 | 2.57 | 2.05 |
| 1101 | ATLVT012XX997 | 45931 | NSQTGGASNDNA | 2.12 | 2.19 | 0.79 |
| 1102 | ATLVT012XX2982 | 47916 | TSATAASSNDNT | 2.45 | 1.98 | 0.49 |
| 1103 | ATLVT012XX767 | 45701 | NSEAGGSTNDNH | 2.11 | 1.97 | 1.07 |
| 1104 | ATLVT012XX2394 | 47328 | TGASGGSSNDNT | 1.44 | 0.71 | 3.84 |
| 1105 | ATLVT012XX2219 | 47153 | SSTSAGASNDNH | 1.11 | 1.18 | 3.79 |
| 1106 | ATLVT012XX404 | 45338 | NGQTAGSSNDNH | 0.5 | 5.25 | −0.41 |
| 1107 | ATLVT012XX1277 | 46211 | SGATGASSNDNH | −0.09 | 6.48 | −0.98 |
| 1108 | ATLVT012XX1691 | 46625 | SGTTAGASNDNH | 0.21 | 1.56 | 4.84 |
| 1109 | ATLVT012XX1838 | 46772 | SSATAGATNDNH | 3.49 | 1.21 | −0.35 |
| 1110 | ATLVT012XX3329 | 48263 | TSTAAGSSNDNH | 3.51 | 0.69 | 0.28 |
| 1111 | ATLVT012XX1861 | 46795 | SSATGGATNDNA | 1.96 | 2.34 | 0.82 |
| 1112 | ATLVT012XX399 | 45333 | NGQTAGASNDNT | 4.04 | 1.09 | −1.14 |
| 1113 | ATLVT012XX785 | 45719 | NSESAGATNDNH | 1.51 | 3.08 | 0.64 |
| 1114 | ATLVT012XX3439 | 48373 | TSTTGASINDNA | 0.81 | 2.57 | 2.48 |
| 1115 | ATLVT012XX1755 | 46689 | SSAAGAATNDNT | 1.74 | 1.67 | 2.04 |
| 1116 | ATLVT012XX1851 | 46785 | SSATGAATNDNT | 3.41 | −0.13 | 1.49 |
| 1117 | ATLVT012XX3010 | 47944 | TSATGGASNDNA | 4.42 | 0.43 | −0.96 |
| 1118 | ATLVT012XX3189 | 48123 | TSQAAGSINDNT | 1.28 | 1.63 | 2.86 |
| 1119 | ATLVT012XX2853 | 47787 | TGTTAGSINDNT | 0.19 | 2.17 | 4.05 |
| 1120 | ATLVT012XX511 | 45445 | NGTSGASSNDNA | 1.68 | 0.42 | 3.74 |
| 1121 | ATLVT012XX2730 | 47664 | TGQTGGSSNDNT | 1.61 | 0.99 | 3.11 |
| 1122 | ATLVT012XX2004 | 46938 | SSETGGASNDNT | 1.48 | 2.23 | 1.74 |
| 1123 | ATLVT012XX3003 | 47937 | TSATGAATNDNT | 3.34 | 0.54 | 0.73 |
| 1124 | ATLVT012XX508 | 45442 | NGTSGAATNDNA | 1.45 | 1.22 | 3.07 |
| 1125 | ATLVT012XX283 | 45217 | NGETGGSSNDNA | −1.18 | 3.68 | 4.41 |
| 1126 | ATLVT012XX2939 | 47873 | TSASAGASNDNH | 2.73 | 1.5 | 0.5 |
| 1127 | ATLVT012XX3209 | 48143 | TSQAGGSSNDNH | 1.6 | 1.77 | 2.07 |
| 1128 | ATLVT012XX1033 | 45967 | NSTAGAASNDNA | 1.38 | 1.44 | 2.86 |
| 1129 | ATLVT012XX3058 | 47992 | TSEAGGASNDNA | 0.1 | 0.42 | 6.37 |
| 1130 | ATLVT012XX1956 | 46890 | SSESGGASNDNT | 1.8 | 2.36 | 0.96 |
| 1131 | ATLVT012XX98 | 45032 | NGATAAASNDNH | 3.48 | 1.17 | −0.4 |
| 1132 | ATLVT012XX1911 | 46845 | SSEAGGAINDNT | 1.76 | 1.47 | 2.18 |
| 1133 | ATLVT012XX3211 | 48145 | TSQAGGSTNDNA | 3.01 | −0.67 | 2.77 |
| 1134 | ATLVT012XX1022 | 45956 | NSTAAGASNDNH | 1.48 | 1.18 | 3 |
| 1135 | ATLVT012XX1358 | 46292 | SGESAGAINDNH | 1.99 | 2.36 | 0.6 |
| 1136 | ATLVT012XX3266 | 48200 | TSQTAAATNDNH | 2.18 | 1.52 | 1.37 |
| 1137 | ATLVT012XX1058 | 45992 | NSTSAAASNDNH | 0.9 | 1.39 | 3.71 |
| 1138 | ATLVT012XX3183 | 48117 | TSQAAGAINDNT | 1.3 | 1.77 | 2.53 |
| 1139 | ATLVT012XX2607 | 47541 | TGQAAGAINDNT | 0.74 | 2.05 | 3.13 |
| 1140 | ATLVT012XX550 | 45484 | NGTTAGSINDNA | 2.06 | 0.52 | 2.83 |
| 1141 | ATLVT012XX3314 | 48248 | TSTAAAATNDNH | −0.89 | 6.3 | 0.44 |
| 1142 | ATLVT012XX708 | 45642 | NSATGASINDNT | 0.88 | 4.71 | −0.55 |
| 1143 | ATLVT012XX768 | 45702 | NSEAGGSINDNT | 1.9 | 2.68 | 0.31 |
| 1144 | ATLVT012XX710 | 45644 | NSATGGASNDNH | 1.98 | 1.72 | 1.42 |
| 1145 | ATLVT012XX2922 | 47856 | TSAAGGSSNDNT | 0.55 | 2.74 | 2.55 |
| 1146 | ATLVT012XX856 | 45790 | NSETGGATNDNA | 0.6 | 5.3 | −0.85 |
| 1147 | ATLVT012XX1924 | 46858 | SSESAASSNDNA | 2.88 | 0.94 | 0.82 |
| 1148 | ATLVT012XX2285 | 47219 | SSTTGASSNDNH | 2.35 | 2.35 | −0.09 |
| 1149 | ATLVT012XX1463 | 46397 | SGQAGAASNDNH | 3.21 | 0.39 | 0.95 |
| 1150 | ATLVT012XX2312 | 47246 | TGAAAASINDNH | 0.77 | 2.62 | 2.26 |
| 1151 | ATLVT012XX1023 | 45957 | NSTAAGASNDNT | 2.36 | 0.45 | 2.33 |
| 1152 | ATLVT012XX2276 | 47210 | SSTTAGSTNDNH | 2.05 | 1.82 | 1.08 |
| 1153 | ATLVT012XX506 | 45440 | NGTSGAASNDNH | 2.7 | 1.22 | 0.74 |
| 1154 | ATLVT012XX2584 | 47518 | TGETGGSSNDNA | 1.88 | 1.55 | 1.71 |
| 1155 | ATLVT012XX2391 | 47325 | TGASGGATNDNT | 0.91 | 2.03 | 2.76 |
| 1156 | ATLVT012XX1339 | 46273 | SGEAGGSINDNA | 2.04 | 1.75 | 1.16 |
| 1157 | ATLVT012XX2762 | 47696 | TGTAGAATNDNH | 1.56 | 1.05 | 2.89 |
| 1158 | ATLVT012XX1016 | 45950 | NSTAAASSNDNH | 0.29 | 4.28 | 0.91 |
| 1159 | ATLVT012XX2585 | 47519 | TGETGGSSNDNH | 2.74 | 0.46 | 1.62 |
| 1160 | ATLVT012XX1567 | 46501 | SGQTGASTNDNA | 1.93 | 0.98 | 2.32 |
| 1161 | ATLVT012XX407 | 45341 | NGQTAGSINDNH | 3.36 | 0.76 | 0.15 |
| 1162 | ATLVT012XX3428 | 48362 | TSTTAGSINDNH | 0.65 | 1.88 | 3.35 |
| 1163 | ATLVT012XX1063 | 45997 | NSTSAASSNDNA | −0.2 | 2.94 | 3.45 |
| 1164 | ATLVT012XX906 | 45840 | NSQAGGATNDNT | 2.99 | 1.2 | 0.21 |
| 1165 | ATLVT012XX3157 | 48091 | TSETGGATNDNA | 1.35 | 4.44 | −1.13 |
| 1166 | ATLVT012XX3038 | 47972 | TSEAAGATNDNH | −0.37 | 1.46 | 5.64 |
| 1167 | ATLVT012XX880 | 45814 | NSQAAGATNDNA | 0.93 | 2.29 | 2.33 |
| 1168 | ATLVT012XX462 | 45396 | NGTAGAATNDNT | 1.1 | 0.97 | 3.75 |
| 1169 | ATLVT01XXX123 | 45057 | NGATGAASNDNT | 3.94 | 0.05 | 0.05 |
| 1170 | ATLVT012XX1274 | 46208 | SGATGAATNDNH | 2.38 | 2 | 0.22 |
| 1171 | ATLVT012XX2810 | 47744 | TGTSGAATNDNH | 1.78 | 2.47 | 0.65 |
| 1172 | ATLVT012XX2515 | 47449 | TGESAGSTNDNA | 2.49 | 1.04 | 1.23 |
| 1173 | ATLVT012XX2609 | 47543 | TGQAAGSSNDNH | 0.95 | 1.77 | 2.93 |
| 1174 | ATLVT012XX1946 | 46880 | SSESGAATNDNH | 1.97 | 2.11 | 0.75 |
| 1175 | ATLVT012XX1656 | 46590 | SGTSGAASNDNT | 2.16 | 1.1 | 1.71 |
| 1176 | ATLVT012XX1307 | 46241 | SGEAAGASNDNH | 2.69 | 0.96 | 0.98 |
| 1177 | ATLVT012XX1647 | 46581 | SGTSAGATNDNT | 2.15 | 0.9 | 1.98 |
| 1178 | ATLVT012XX1380 | 46314 | SGESGGASNDNT | 1.34 | 1.08 | 3.12 |
| 1179 | ATLVT012XX1705 | 46639 | SGTTGAATNDNA | 2.59 | 0.54 | 1.65 |
| 1180 | ATLVT012XX215 | 45149 | NGESAGSTNDNH | −0.28 | 5.53 | 0.17 |
| 1181 | ATLVT012XX3037 | 47971 | TSEAAGAINDNA | −0.3 | 3.65 | 2.59 |
| 1182 | ATLVT012XX2816 | 47750 | TGTSGASTNDNH | 0.8 | 3.48 | 0.91 |
| 1183 | ATLVT012XX552 | 45486 | NGTTAGSINDNT | −0.11 | 1.1 | 5.54 |
| 1184 | ATLVT012XX2904 | 47838 | TSAAGAASNDNT | 1.2 | 1.87 | 2.3 |
| 1185 | ATLVT012XX2334 | 47268 | TGAAGASSNDNT | 0.69 | 2.07 | 2.88 |
| 1186 | ATLVT012XX2426 | 47360 | TGATGAATNDNH | 1.01 | 2 | 2.4 |
| 1187 | ATLVT012XX1517 | 46451 | SGQSGASSNDNH | 0.96 | 2.56 | 1.78 |
| 1188 | ATLVT012XX1659 | 46593 | SGTSGAATNDNT | 1.62 | 0.37 | 3.45 |
| 1189 | ATLVT012XX2837 | 47771 | TGTTAASSNDNH | 2.12 | 1.63 | 0.98 |
| 1190 | ATLVT012XX1026 | 45960 | NSTAAGAINDNT | 1.32 | 1.2 | 2.86 |
| 1191 | ATLVT012XX1699 | 46633 | SGTTAGSINDNA | 0.21 | 2.88 | 2.6 |
| 1192 | ATLVT012XX2917 | 47851 | TSAAGGATNDNA | 0.62 | 2.47 | 2.41 |
| 1193 | ATLVT012XX2797 | 47731 | TGTSAGATNDNA | 2.66 | 0.64 | 1.27 |
| 1194 | ATLVT012XX1160 | 46094 | SGAAAASINDNH | 0.09 | 4.47 | 0.74 |
| 1195 | ATLVT012XX2942 | 47876 | TSASAGATNDNH | 1.25 | 2.86 | 0.83 |
| 1196 | ATLVT012XX3342 | 48276 | TSTAGASSNDNT | 3.29 | 0.72 | 0.08 |
| 1197 | ATLVT012XX2188 | 47122 | SSTAGASSNDNA | 1.22 | 1.38 | 2.77 |
| 1198 | ATLVT012XX161 | 45095 | NGEAAGATNDNH | 0.41 | −0.02 | 5.95 |
| 1199 | ATLVT012XX238 | 45172 | NGESGGSINDNA | 2.35 | 2.55 | −0.67 |
| 1200 | ATLVT012XX421 | 45355 | NGQTGGASNDNA | −0.52 | 3.52 | 2.99 |
| 1201 | ATLVT012XX1696 | 46630 | SGTTAGSSNDNA | 0.28 | 2.77 | 2.59 |
| 1202 | ATLVT012XX2733 | 47667 | TGQTGGSINDNT | −0.01 | 1.79 | 4.33 |
| 1203 | ATLVT012XX2409 | 47343 | TGATAASTNDNT | −0.34 | 5.2 | 0.51 |
| 1204 | ATLVT012XX3440 | 48374 | TSTTGASINDNH | 1.63 | 1.78 | 1.52 |
| 1205 | ATLVT012XX872 | 45806 | NSQAAASSNDNH | −1.15 | 6.42 | 0.31 |
| 1206 | ATLVT012XX848 | 45782 | NSETGASSNDNH | 0.96 | 4.76 | −1.19 |
| 1207 | ATLVT012XX302 | 45236 | NGQAAGASNDNH | 0.59 | 2.17 | 2.76 |
| 1208 | ATLVT012XX547 | 45481 | NGTTAGSSNDNA | 1.08 | 0.22 | 4.42 |
| 1209 | ATLVT012XX1451 | 46385 | SGQAAGASNDNH | 0.58 | 2.49 | 2.36 |
| 1210 | ATLVT012XX542 | 45476 | NGTTAGASNDNH | 0.55 | 0.66 | 4.77 |
| 1211 | ATLVT012XX1226 | 46160 | SGASGAATNDNH | 1.65 | 2.2 | 0.87 |
| 1212 | ATLVT012XX1685 | 46619 | SGTTAASSNDNH | 1.18 | 2.77 | 0.94 |
| 1213 | ATLVT012XX2759 | 47693 | TGTAGAASNDNH | 1.02 | 2.17 | 2 |
| 1214 | ATLVT012XX1860 | 46794 | SSATGGASNDNT | 2.72 | 0.99 | 0.56 |
| 1215 | ATLVT012XX43 | 44977 | NGAAGGSSNDNA | 1.23 | 1.4 | 2.6 |
| 1216 | ATLVT012XX839 | 45773 | NSETAGSINDNH | 2.04 | 0.06 | 2.92 |
| 1217 | ATLVT012XX707 | 45641 | NSATGASTNDNH | 0.4 | 5.12 | −0.78 |
| 1218 | ATLVT012XX365 | 45299 | NGQSGAATNDNH | 2.43 | 1.08 | 0.94 |
| 1219 | ATLVT012XX901 | 45835 | NSQAGGASNDNA | 2.4 | 1.12 | 0.93 |
| 1220 | ATLVT012XX2909 | 47843 | TSAAGASSNDNH | 0.54 | 2.87 | 1.87 |
| 1221 | ATLVT012XX2009 | 46943 | SSETGGSSNDNH | 3.03 | 0.73 | 0.35 |
| 1222 | ATLVT012XX2489 | 47423 | TGEAGGSSNDNH | 4.4 | −0.32 | −0.66 |
| 1223 | ATLVT012XX604 | 45538 | NSAAGAATNDNA | 0.78 | 2.02 | 2.53 |
| 1224 | ATLVT012XX2501 | 47435 | TGESAASSNDNH | −0.51 | 5.8 | −0.13 |
| 1225 | ATLVT012XX320 | 45254 | NGQAGASSNDNH | 1.86 | 2.06 | 0.61 |
| 1226 | ATLVT012XX2395 | 47329 | TGASGGSTNDNA | 1.47 | 0.76 | 2.94 |
| 1227 | ATLVT012XX1473 | 16407 | SGQAGASTNDNT | 0.42 | 1.85 | 3.32 |
| 1228 | ATLVT012XX1552 | 46486 | SGQTAGSSNDNA | 3.01 | 1.08 | −0.13 |
| 1229 | ATLVT012XX1735 | 46669 | SSAAAASTNDNA | −0.64 | 2.03 | 4.91 |
| 1230 | ATLVT012XX2444 | 47378 | TGATGGSTNDNH | 1.25 | 1.79 | 1.98 |
| 1231 | ATLVT012XX2799 | 47733 | TGTSAGAINDNT | 2.72 | 0.26 | 1.42 |
| 1232 | ATLVT012XX3091 | 48025 | TSESAGSINDNA | −2.29 | 5.3 | 3.54 |
| 1233 | ATLVT012XX3208 | 48142 | TSQAGGSSNDNA | 0.13 | 0.57 | 5.44 |
| 1234 | ATLVT012XX3181 | 48115 | TSQAAGATNDNA | 0.61 | 2.21 | 2.52 |
| 1235 | ATLVT012XX984 | 45918 | NSQTAGSINDNT | 3.12 | −0.01 | 1.06 |
| 1236 | ATLVT012XX674 | 45608 | NSATAAASNDNH | 0.98 | 0.4 | 4.18 |
| 1237 | ATLVT012XX1361 | 46295 | SGESAGSSNDNH | 0.24 | 4.92 | −0.37 |
| 1238 | ATLVT012XX1836 | 46770 | SSATAGASNDNT | 0.09 | 4.9 | −0.07 |
| 1239 | ATLVT012XX651 | 45585 | NSASGAASNDNT | 2.18 | 1.08 | 1.25 |
| 1240 | ATLVT012XX620 | 45554 | NSAAGGSSNDNH | −0.08 | 3.69 | 1.77 |
| 1241 | ATLVT012XX2414 | 47348 | TGATAGATNDNH | 2.29 | 0.57 | 1.7 |
| 1242 | ATLVT012XX2838 | 47772 | TGTTAASSNDNT | −0.31 | 2.94 | 3.11 |
| 1243 | ATLVT01XXX1019 | 45953 | NSTAAASINDNH | 3.27 | 0.77 | −0.25 |
| 1244 | ATLVT012XX2366 | 47300 | IGASAGAINDNH | 0.88 | 2.1 | 2.14 |
| 1245 | ATLVT012XX2360 | 47294 | TGASAASINDNH | −0.76 | 5.95 | −0.01 |
| 1246 | ATLVT012XX1059 | 45993 | NSTSAAASNDNT | 0.96 | 0.38 | 4.17 |
| 1247 | ATLVT012XX536 | 45470 | NGTTAASSNDNH | 1.71 | 1.95 | 0.87 |
| 1248 | ATLVT012XX1657 | 46591 | SGTSGAATNDNA | 1.42 | 0.86 | 2.77 |
| 1249 | ATLVT012XX2921 | 47855 | TSAAGGSSNDNH | 1.17 | 2 | 1.72 |
| 1250 | ATLVT012XX1899 | 46833 | SSEAGAATNDNT | 1.66 | 1.08 | 2.06 |
| 1251 | ATLVT012XX2439 | 47373 | TGATGGAINDNT | −0.19 | 2.96 | 2.82 |
| 1252 | ATLVT012XX2955 | 47889 | TSASGAATNDNT | 1.22 | 1.26 | 2.57 |
| 1253 | ATLVT012XX2531 | 47465 | TGESGGASNDNH | −0.5 | 4.01 | 1.98 |
| 1254 | ATLVT012XX2189 | 47123 | SSTAGASSNDNH | 2.21 | 1.01 | 1.2 |
| 1255 | ATLVT012XX2751 | 47685 | TGTAAGAINDNT | 1.72 | 1.3 | 1.65 |
| 1256 | ATLVT012XX28 | 44962 | NGAAGAATNDNA | 0.74 | 1.83 | 2.64 |
| 1257 | ATLVT012XX650 | 45584 | NSASGAASNDNH | 2.41 | 0.94 | 0.91 |
| 1258 | ATLVT012XX118 | 45052 | NGATAGSINDNA | 3.96 | −0.66 | 0.3 |
| 1259 | ATLVT012XX888 | 45822 | NSQAAGSINDNT | 0.74 | 2.53 | 1.73 |
| 1260 | ATLVT012XX1742 | 46676 | SSAAAGAINDNH | 1.34 | 1.93 | 1.47 |
| 1261 | ATLVT012XX2434 | 47368 | TGATGGASNDNA | 1.35 | 0.94 | 2.72 |
| 1262 | ATLVT012XX2136 | 47070 | SSQTGAASNDNT | 2.51 | −0.55 | 2.64 |
| 1263 | ATLVT012XX1902 | 46836 | SSEAGASSNDNT | 0.44 | −0.63 | 6.29 |
| 1264 | ATLVT012XX709 | 45643 | NSATGGASNDNA | 1.24 | 1.07 | 2.73 |
| 1265 | ATLVT012XX1934 | 46868 | SSESAGAINDNH | −0.32 | 5.02 | 0.31 |
| 1266 | ATLVT012XX884 | 45818 | NSQAAGSSNDNH | 0.86 | 2.29 | 1.8 |
| 1267 | ATLVT012XX2834 | 47768 | TGTTAAATNDNH | 0.44 | 4.79 | −0.71 |
| 1268 | ATLVT012XX1602 | 46536 | SGTAAGSSNDNT | 1.7 | 2.17 | 0.5 |
| 1269 | ATLVT012XX2915 | 47849 | TSAAGGASNDNH | 0.71 | 2.13 | 2.25 |
| 1270 | ATLVT012XX595 | 45529 | NSAAAGSSNDNA | −0.71 | 5.94 | −0.22 |
| 1271 | ATLVT012XX3195 | 48129 | TSQAGAATNDNT | 0.6 | 2.32 | 2.18 |
| 1272 | ATLVT012XX1501 | 46435 | SGQSAGATNDNA | 2.98 | 1.33 | −0.63 |
| 1273 | ATLVT012XX1897 | 46831 | SSEAGAATNDNA | 2.75 | 0.23 | 1.16 |
| 1274 | ATLVT012XX2035 | 46969 | SSQAAGSTNDNA | 1.35 | 1.86 | 1.47 |
| 1275 | ATLVT012XX2113 | 47047 | SSQTAAATNDNA | 0.93 | 0.34 | 4.11 |
| 1276 | ATLVT012XX2118 | 47052 | SSQTAASSNDNT | 1.31 | −1.2 | 5.45 |
| 1277 | ATLVT012XX1017 | 45951 | NSTAAASSNDNT | 1.72 | 0.7 | 2.3 |
| 1278 | ATLVT012XX3065 | 47999 | TSEAGGSSNDNH | 3.43 | −0.27 | 0.61 |
| 1279 | ATLVT012XX2097 | 47031 | SSQSGASTNDNT | 2.2 | −0.32 | 2.77 |
| 1280 | ATLVT012XX1655 | 46589 | SGTSGAASNDNH | 1.95 | 0.84 | 1.69 |
| 1281 | ATLVT012XX84 | 45018 | NGASGASTNDNT | 2.79 | 0.31 | 0.91 |
| 1282 | ATLVT012XX945 | 45879 | NSQSGASSNDNT | 0.59 | 1.49 | 3.17 |
| 1283 | ATLVT012XX1692 | 46626 | SGTTAGASNDNT | 0.57 | 0.99 | 3.84 |
| 1284 | ATLVT012XX496 | 45430 | NGTSAGATNDNA | 1.11 | 3.35 | −0.11 |
| 1285 | ATLVT012XX1605 | 46539 | SGTAAGSINDNT | 1.18 | 2.2 | 1.23 |
| 1286 | ATLVT012XX2758 | 47692 | TGTAGAASNDNA | 1.49 | 0.69 | 2.64 |
| 1287 | ATLVT012XX2800 | 47734 | TGTSAGSSNDNA | 2.87 | 0.56 | 0.43 |
| 1288 | ATLVT012XX1224 | 46158 | SGASGAASNDNT | 1.94 | 1.04 | 1.41 |
| 1289 | ATLVT012XX141 | 45075 | NGATGGSSNDNT | 1.34 | 0.63 | 2.97 |
| 1290 | ATLVT012XX2666 | 47600 | TGQSGAATNDNH | 0.62 | 2.83 | 1.37 |
| 1291 | ATLVT012XX2760 | 47694 | TGTAGAASNDNT | 1.73 | 1.28 | 1.45 |
| 1292 | ATLVT012XX2270 | 47204 | SSTTAGATNDNH | 0.78 | 1.97 | 2.19 |
| 1293 | ATLVT012XX2893 | 47827 | TSAAAGAINDNA | 0.39 | 3.47 | 0.94 |
| 1294 | ATLVT012XX3062 | 47996 | TSEAGGAINDNH | 0.52 | 3.27 | 0.96 |
| 1295 | ATLVT012XX1738 | 46672 | SSAAAGASNDNA | 0.59 | 2.41 | 1.95 |
| 1296 | ATLVT012XX2346 | 47280 | TGAAGGSSNDNT | 0.17 | 1.98 | 3.22 |
| 1297 | ATLVT012XX1024 | 45958 | NSTAAGATNDNA | 1.25 | 1.45 | 2.04 |
| 1298 | ATLVT012XX468 | 45402 | NGTAGASINDNT | 3.26 | −0.56 | 1.17 |
| 1299 | ATLVT012XX578 | 45512 | NSAAAAASNDNH | 3.48 | −0.59 | 0.83 |
| 1300 | ATLVT012XX2999 | 47933 | TSATGAASNDNH | 1.86 | 1.29 | 1.19 |
| 1301 | ATLVT012XX3391 | 48325 | TSTSGASTNDNA | 0.11 | 2.53 | 2.58 |
| 1302 | ATLVT012XX2281 | 47215 | SSTTGAATNDNA | 1.23 | 2.08 | 1.25 |
| 1303 | ATLVT012XX2756 | 47690 | TGTAAGSINDNH | 1.65 | 0.81 | 2.15 |
| 1304 | ATLVT012XX891 | 45825 | NSQAGAASNDNT | 3.12 | 0.41 | 0.13 |
| 1305 | ATLVT012XX2126 | 47060 | SSQTAGATNDNH | 1.32 | 1.18 | 2.23 |
| 1306 | ATLVT012XX482 | 45416 | NGTSAAASNDNH | 0.76 | 0.99 | 3.42 |
| 1307 | ATLVT012XX2811 | 47745 | TGTSGAATNDNT | 1.02 | 1.71 | 2.05 |
| 1308 | ATLVT012XX3161 | 48095 | TSETGGSSNDNH | 3.3 | 0.52 | −0.33 |
| 1309 | ATLVT012XX427 | 45361 | NGQTGGSSNDNA | 0.81 | 3.01 | 0.74 |
| 1310 | ATLVT012XX3359 | 48293 | TSTSAAASNDNH | −1.57 | 5.33 | 1.82 |
| 1311 | ATLVT012XX1882 | 46816 | SSEAAGASNDNA | 0.33 | 0.57 | 4.68 |
| 1312 | ATLVT012XX924 | 45858 | NSQSAASINDNT | 2.16 | −1.23 | 3.86 |
| 1313 | ATLVT012XX803 | 45737 | NSESGASTNDNH | 3.34 | 0.71 | −0.66 |
| 1314 | ATLVT012XX2037 | 46971 | SSQAAGSINDNT | 0.47 | 1.74 | 2.93 |
| 1315 | ATLVT012XX2763 | 47697 | TGTAGAATNDNT | 0.8 | 1.03 | 3.28 |
| 1316 | ATLVT012XX1607 | 46541 | SGTAGAASNDNH | 2.12 | 1.31 | 0.65 |
| 1317 | ATLVT012XX3265 | 48199 | TSQTAAATNDNA | 2.43 | −0.48 | 2.4 |
| 1318 | ATLVT012XX3322 | 48256 | TSTAAGASNDNA | 0.49 | 2.62 | 1.73 |
| 1319 | ATLVT012XX1547 | 4648] | SGQTAGASNDNH | 0.23 | 2.85 | 1.86 |
| 1320 | ATLVT012XX1613 | 46547 | SGTAGASSNDNH | 1.68 | 0.89 | 1.89 |
| 1321 | ATLVT012XX3187 | 48121 | TSQAAGSTNDNA | 0.81 | 1.34 | 2.8 |
| 1322 | ATLVT012XX45 | 44979 | NGAAGGSSNDNT | 0.56 | 1.84 | 2.58 |
| 1323 | ATLVT012XX1704 | 46638 | SGTTGAASNDNT | 2.34 | 0.14 | 1.71 |
| 1324 | ATLVT012XX1615 | 46549 | SGTAGASTNDNA | 1.75 | 0.4 | 2.38 |
| 1325 | ATLVT012XX2278 | 47212 | SSTTGAASNDNA | 3.05 | 0.77 | −0.34 |
| 1326 | ATLVT012XX1229 | 46163 | SGASGASSNDNH | 1.26 | 1.68 | 1.56 |
| 1327 | ATLVT012XX786 | 45720 | NSESAGAINDNT | 0.51 | 3.77 | 0.16 |
| 1328 | ATLVT012XX319 | 45253 | NGQAGASSNDNA | 3.71 | −0.69 | 0.4 |
| 1329 | ATLVT012XX2510 | 47444 | TGESAGATNDNH | −0.93 | 4.77 | 1.33 |
| 1330 | ATLVT012XX980 | 45914 | NSQTAGSSNDNH | 1.65 | 1.28 | 1.38 |
| 1331 | ATLVT012XX1027 | 45961 | NSTAAGSSNDNA | 1.87 | 0.98 | 1.39 |
| 1332 | ATLVT012XX1513 | 46447 | SGQSGAATNDNA | 2.56 | 0 | 1.46 |
| 1333 | ATLVT012XX3064 | 47998 | TSEAGGSSNDNA | 2.22 | 0.52 | 1.38 |
| 1334 | ATLVT012XX2887 | 47821 | TSAAAASTNDNA | −1.7 | 5.51 | 1.68 |
| 1335 | ATLVT012XX946 | 45880 | NSQSGASINDNA | 1.69 | 1.49 | 1.03 |
| 1336 | ATLVT012XX3345 | 48279 | TSTAGASTNQNT | 1.05 | 0.11 | 3.9 |
| 1337 | ATLVT012XX1310 | 46244 | SGEAAGATNDNH | 0.34 | 1.79 | 2.96 |
| 1338 | ATLVT012XX2950 | 47884 | TSASGAASNDNA | 1.48 | 0.71 | 2.39 |
| 1339 | ATLVT012XX2442 | 47376 | TGATGGSSNDNT | 1.12 | 0.69 | 3 |
| 1340 | ATLVT012XX2013 | 46947 | SSETGGSINDNT | 1.98 | 0.99 | 1.14 |
| 1341 | ATLVT012XX1516 | 46450 | SGQSGASSNDNA | 1.41 | 1.16 | 1.9 |
| 1342 | ATLVT012XX2071 | 47005 | SSQSAASINDNA | 2.68 | −0.04 | 1.26 |
| 1343 | ATLVT012XX3375 | 48309 | TSTSAGATNDNT | −0.48 | 4.03 | 1.45 |
| 1344 | ATLVT012XX3056 | 47990 | TSEAGASTNDNH | 3.2 | 0.21 | 0.03 |
| 1345 | ATLVT012XX1173 | 46107 | SGAAAGSINDNT | 0.77 | 1.35 | 2.72 |
| 1346 | ATLVT012XX3001 | 47935 | TSATGAATNDNA | 3.25 | 0.1 | 0.07 |
| 1347 | ATLVT012XX1796 | 46730 | SSASAGSTNDNH | 2.59 | 2.18 | −1.49 |
| 1348 | ATLVT012XX1791 | 46725 | SSASAGATNDNT | 3.11 | 0.94 | −0.8 |
| 1349 | ATLVT012XX443 | 45377 | NGTAAASTNDNH | 1.53 | 1.49 | 1.2 |
| 1350 | ATLVT012XX2685 | 47619 | TGQSGGSTNDNT | −0.17 | 3.5 | 1.53 |
| 1351 | ATLVT012XX303 | 45237 | NGQAAGASNDNT | 1.14 | 1.26 | 2.14 |
| 1352 | ATLVT012XX500 | 45434 | NGTSAGSSNDNH | 1.8 | 1.87 | 0.23 |
| 1353 | ATLVT012XX2092 | 47026 | SSQSGASSNDNA | 2.13 | 0.49 | 1.44 |
| 1354 | ATLVT012XX886 | 45820 | NSQAAGSTNDNA | 0.58 | 2.22 | 1.87 |
| 1355 | ATLVT012XX851 | 45785 | NSETGASTNDNH | 2.88 | 1 | −0.5 |
| 1356 | ATLVT012XX513 | 45447 | NGTSGASSNDNT | 0.34 | 0.77 | 4.14 |
| 1357 | ATLVT012XX3213 | 48147 | TSQAGGSINDNT | 0.97 | 0.55 | 3.33 |
| 1358 | ATLVT012XX797 | 45731 | NSESGAATNDNH | 2.31 | 0.22 | 1.46 |
| 1359 | ATLVT012XX1684 | 46618 | SGTTAASSNDNA | 1.44 | 1.35 | 1.5 |
| 1360 | ATLVT012XX2803 | 47737 | TGTSAGSTNDNA | 2.55 | 0.08 | 1.22 |
| 1361 | ATLVT012XX2661 | 47595 | TGQSAGSINDNT | 1.67 | 2.48 | −0.36 |
| 1362 | ATLVT012XX1268 | 46202 | SGATAGSTNDNH | 1.99 | 1.64 | 0.17 |
| 1363 | ATLVT012XX1947 | 46881 | SSESGAATNDNT | 3.23 | −0.38 | 0.62 |
| 1364 | ATLVT012XX2840 | 47774 | TGTTAASTNDNH | 0.25 | 3.91 | 0.2 |
| 1365 | ATLVT012XX2977 | 47911 | TSATAAATNDNA | 0.18 | 4.56 | −0.54 |
| 1366 | ATLVT012XX2456 | 47390 | TGEAAASTNDNH | 1.77 | 2.21 | −0.25 |
| 1367 | ATLVT012XX2174 | 47108 | SSTAAGATNDNH | −0.22 | 2.98 | 2.17 |
| 1368 | ATLVT012XX2954 | 47888 | TSASGAATNDNH | 1.46 | 0.86 | 2.02 |
| 1369 | ATLVT012XX2123 | 47057 | SSQTAGASNDNH | 0.03 | −0.05 | 5.62 |
| 1370 | ATLVT012XX2970 | 47904 | TSASGGSSNDNT | 2.01 | 0.08 | 2.07 |
| 1371 | ATLVT012XX2468 | 47402 | TGEAAGSINDNH | 1.36 | 1.18 | 1.75 |
| 1372 | ATLVT012XX1894 | 46828 | SSEAGAASNDNA | 0.28 | −0.52 | 5.79 |
| 1373 | ATLVT012XX1179 | 46113 | SGAAGAATNDNT | 0.61 | 1.28 | 2.91 |
| 1374 | ATLVT012XX2850 | 47784 | TGTTAGSSNDNT | −0.7 | 4.32 | 1.24 |
| 1375 | ATLVT012XX80 | 45014 | NGASGASSNDNH | −0.45 | 0.27 | 6.01 |
| 1376 | ATLVT012XX2010 | 46944 | SSETGGSSNDNT | 2.3 | 0.34 | 1.21 |
| 1377 | ATLVT012XX1631 | 46565 | SGTSAAASNDNH | 0.93 | 0.08 | 3.87 |
| 1378 | ATLVT012XX3255 | 48189 | TSQSGGATNDNT | 0.58 | 0.6 | 3.81 |
| 1379 | ATLVT012XX1808 | 46742 | SSASGASINDNH | 1.23 | 1.37 | 1.71 |
| 1380 | ATLVT012XX3239 | 48173 | TSQSGAASNDNH | 1.09 | 0.88 | 2.56 |
| 1381 | ATLVT012XX2629 | 47563 | IGQAGGATNDNA | 0.81 | −0.27 | 4.51 |
| 1382 | ATLVT012XX1395 | 46329 | SGETAAATNDNT | 1.96 | 1.6 | 0.15 |
| 1383 | ATLVT012XX1488 | 46422 | SGQSAAASNDNT | 2.64 | 0.06 | 0.95 |
| 1384 | ATLVT012XX3275 | 48209 | TSQTAGASNDNH | 0.38 | 2.09 | 2.2 |
| 1385 | ATLVT012XX2941 | 47875 | TSASAGAINDNA | −0.1 | 3.81 | 0.76 |
| 1386 | ATLVT012XX235 | 45169 | NGESGGSSNDNA | 0.57 | 2.9 | 0.78 |
| 1387 | ATLVT012XX2480 | 47414 | TGEAGASINDNH | 3.95 | −0.3 | −0.91 |
| 1388 | ATLVT012XX2178 | 47112 | SSTAAGSSNDNT | 0.2 | 0.24 | 4.84 |
| 1389 | ATLVT012XX2663 | 47597 | TGQSGAASNDNH | 1.24 | 1.71 | 1.15 |
| 1390 | ATLVT012XX2635 | 47569 | TGQAGGSTNDNA | 0.51 | 0.22 | 4.29 |
| 1391 | ATLVT012XX2749 | 47683 | TGTAAGATNDNA | 0.67 | 2.56 | 1.01 |
| 1392 | ATLVT012XX798 | 45732 | NSESGAATNDNT | 0.88 | 0.72 | 3.02 |
| 1393 | ATLVT012XX1844 | 46778 | SSATAGSTNDNH | 1.76 | 1.9 | −0.01 |
| 1394 | ATLVT012XX1060 | 45994 | NSTSAAATNDNA | −0.35 | 3.34 | 1.76 |
| 1395 | ATLVT012XX3339 | 48273 | ISTAGAATNDNT | 1.04 | 1.04 | 2.33 |
| 1396 | ATLVT012XX3333 | 48267 | TSTAAGSINDNT | 0.91 | 2.57 | 0.54 |
| 1397 | ATLVT012XX3204 | 48138 | TSQAGGASNDNT | 1.77 | 0.31 | 1.97 |
| 1398 | ATLVT012XX1263 | 46197 | SGATAGATNDNT | 2.98 | −0.42 | 0.84 |
| 1399 | ATLVT012XX940 | 45874 | NSQSGAATNDNA | 1.36 | 1.33 | 1.36 |
| 1400 | ATLVT012XX989 | 45923 | NSQTGAATNDNH | 1.42 | 0.89 | 1.83 |
| 1401 | ATLVT012XX1767 | 46701 | SSAAGGAINDNT | 1 | 1.43 | 1.85 |
| 1402 | ATLVT012XX2491 | 47425 | TGEAGGSINDNA | 1.58 | 2.3 | −0.27 |
| 1403 | ATLVT012XX3340 | 48274 | TSTAGASSNDNA | 0.99 | 2.64 | 0.31 |
| 1404 | ATLVT012XX422 | 45356 | NGQTGGASNDNH | −0.26 | 3.1 | 1.85 |
| 1405 | ATLVT012XX1400 | 46334 | SGETAASTNQNH | 3.01 | 1.07 | −1.16 |
| 1406 | ATLVT012XX2216 | 47150 | SSTSAASTNDNH | 0.84 | 3.34 | −0.37 |
| 1407 | ATLVT012XX2033 | 46967 | SSQAAGSSNDNH | 1.14 | 1.68 | 1.26 |
| 1408 | ATLVT012XX2572 | 47506 | TGETGASSNDNA | 3 | −0.45 | 0.8 |
| 1409 | ATLVT012XX2540 | 47474 | TGESGGSTNDNH | −0.69 | 1.87 | 4.15 |
| 1410 | ATLVT012XX2066 | 47000 | SSQSAAATNDNH | 3.62 | −0.95 | 0.38 |
| 1411 | ATLVT012XX2597 | 47531 | TGQAAASSNDNH | 2.96 | −0.48 | 0.87 |
| 1412 | ATLVT012XX2586 | 47520 | TGETGGSSNDNT | 0.4 | 1.21 | 3.1 |
| 1413 | ATLVT012XX3243 | 48177 | TSQSGAATNDNT | −0.05 | 1.95 | 2.92 |
| 1414 | ATLVT012XX349 | 45283 | NGQSAGASNDNA | −0.43 | −0.48 | 6.68 |
| 1415 | ATLVT012XX1793 | 46727 | SSASAGSSNDNH | 2.88 | 0.39 | −0.11 |
| 1416 | ATLVT012XX2897 | 47831 | TSAAAGSSNDNH | 0.52 | 2.28 | 1.5 |
| 1417 | ATLVT012XX1254 | 46188 | SGATAASSNQNT | 0.75 | 3.01 | 0.17 |
| 1418 | ATLVT012XX234 | 45168 | NGESGGATNDNT | 2.54 | 1.07 | −0.42 |
| 1419 | ATLVT012XX2809 | 47743 | TGTSGAATNDNA | 0.89 | 1.24 | 2.2 |
| 1420 | ATLVT012XX2619 | 47553 | TGQAGAATNDNT | 0.71 | 1.17 | 2.6 |
| 1421 | ATLVT012XX2333 | 47267 | TGAAGASSNDNH | 0.37 | 1.81 | 2.34 |
| 1422 | ATLVT012XX398 | 45332 | NGQTAGASNDNH | 0.17 | 3.96 | −0.08 |
| 1423 | ATLVT012XX3241 | 48175 | TSQSGAATNDNA | 0.46 | 1.58 | 2.48 |
| 1424 | ATLVT012XX2908 | 47842 | TSAAGASSNDNA | 0.94 | 1.56 | 1.67 |
| 1425 | ATLVT012XX3324 | 48258 | TSTAAGASNDNT | 0.68 | 2.15 | 1.36 |
| 1426 | ATLVT012XX3112 | 48046 | TSESGGSSNDNA | −0.55 | 3.68 | 1.49 |
| 1427 | ATLVT012XX2238 | 47172 | SSTSGASSNDNT | 1.26 | 0.77 | 2.13 |
| 1428 | ATLVT012XX1158 | 46092 | SGAAAASSNDNT | 0.8 | 1.67 | 1.78 |
| 1429 | ATLVT012XX323 | 45257 | NGQAGASINDNH | 2.48 | 0.4 | 0.52 |
| 1430 | ATLVT012XX2672 | 47606 | TGQSGASTNDNH | −0.27 | 4.17 | 0.38 |
| 1431 | ATLVT012XX1275 | 46209 | SGATGAATNDNT | 2.24 | 0.5 | 0.79 |
| 1432 | ATLVT012XX3050 | 47984 | TSEAGAATNDNH | 0.65 | 1.74 | 1.93 |
| 1433 | ATLVT012XX3373 | 48307 | TSTSAGATNDNA | 0.5 | 2.04 | 1.79 |
| 1434 | ATLVT012XX2046 | 46980 | SSQAGASSNDNT | 3.19 | −0.96 | 1.04 |
| 1435 | ATLVT012XX794 | 45728 | NSESGAASNDNH | 1.8 | −0.03 | 2.22 |
| 1436 | ATLVT012XX423 | 45357 | NGQTGGASNDNT | 0.75 | 3.22 | −0.17 |
| 1437 | ATLVT012XX2082 | 47016 | SSQSAGSSNDNT | 0.77 | 1.71 | 1.74 |
| 1438 | ATLVT012XX2768 | 47702 | TGTAGASTNDNH | 2.97 | −0.51 | 0.83 |
| 1439 | ATLVT012XX174 | 45108 | NGEAGAATNDNT | 3.47 | −0.88 | 0.43 |
| 1440 | ATLVT012XX2063 | 46997 | SSQSAAASNDNH | 2.48 | −0.99 | 2.27 |
| 1441 | ATLVT012XX1775 | 46709 | SSASAAASNDNH | 2.4 | 0.62 | 0.32 |
| 1442 | ATLVT012XX3435 | 48369 | TSTTGAATNDNT | 2.07 | 0.19 | 1.44 |
| 1443 | ATLVT012XX2116 | 47050 | SSQTAASSNDNA | 1.15 | −0.02 | 3.28 |
| 1444 | ATLVT012XX679 | 45613 | NSATAASSNDNA | 3.74 | −0.76 | −0.2 |
| 1445 | ATLVT012XX558 | 45492 | NGTTGAATNDNT | 1.45 | 1.06 | 1.38 |
| 1446 | ATLVT012XX2408 | 47342 | TGATAASTNDNH | −0.53 | 4.33 | 0.57 |
| 1447 | ATLVT012XX1394 | 46328 | SGETAAAINDNH | 1.54 | 3.03 | −1.33 |
| 1448 | ATLVT012XX1737 | 46671 | SSAAAASTNDNT | 1.42 | 0.22 | 2.5 |
| 1449 | ATLVT012XX35 | 44969 | NGAAGASTNDNH | 1.03 | 1.16 | 1.95 |
| 1450 | ATLVT012XX2624 | 47558 | TGQAGASINDNH | −0.3 | 2.91 | 1.97 |
| 1451 | ATLVT012XX2389 | 47323 | TGASGGATNDNA | 1.15 | 0.65 | 2.4 |
| 1452 | ATLVT012XX1595 | 46529 | SGTAAGASNDNH | 1.12 | 1.89 | 0.85 |
| 1453 | ATLVT012XX230 | 45164 | NGESGGASNDNH | 1.19 | 2.7 | −0.32 |
| 1454 | ATLVT012XX430 | 45364 | NGQTGGSTNDNA | 0.42 | 2.61 | 1.12 |
| 1455 | ATLVT012XX1464 | 46398 | SGQAGAASNDNT | 0.95 | 1.24 | 1.97 |
| 1456 | ATLVT012XX2796 | 47730 | TGTSAGASNDNT | 1.89 | 0.25 | 1.61 |
| 1457 | ATLVT012XX1317 | 46251 | SGEAAGSTNDNT | 3.16 | −0.62 | 0.54 |
| 1458 | ATLVT012XX688 | 45622 | NSATAGAINDNA | −0.1 | 0.31 | 4.94 |
| 1459 | ATLVT012XX2654 | 47588 | TGQSAGATNDNH | 1.54 | 1.61 | 0.44 |
| 1460 | ATLVT012XX2311 | 47245 | TGAAAASTNDNA | 0.59 | 1.41 | 2.32 |
| 1461 | ATLVT012XX308 | 45242 | NGQAAGSSNDNH | 1.1 | 1.39 | 1.47 |
| 1462 | ATLVT012XX1020 | 45954 | NSTAAASTNDNT | 1.07 | 0.47 | 2.71 |
| 1463 | ATLVT012XX1108 | 46042 | NSTTAASSNDNA | 0.82 | −0.07 | 3.82 |
| 1464 | ATLVT012XX494 | 45428 | NGTSAGASNDNH | 1.45 | 0.95 | 1.41 |
| 1465 | ATLVT012XX2318 | 47252 | TGAAAGATNDNH | 0.52 | 1.64 | 2.12 |
| 1466 | ATLVT012XX2269 | 47203 | SSTTAGATNDNA | −0.56 | 1.54 | 4.09 |
| 1467 | ATLVT012XX1102 | 46036 | NSTTAAASNDNA | 1.45 | 2.55 | −0.66 |
| 1468 | ATLVT012XX2795 | 47729 | TGTSAGASNDNH | 2.01 | 0.25 | 1.33 |
| 1469 | ATLVT012XX465 | 45399 | NGTAGASSNDNT | −0.62 | 1.21 | 4.61 |
| 1470 | ATLVT012XX937 | 45871 | NSQSGAASNDNA | 0.3 | 2.07 | 1.92 |
| 1471 | ATLVT012XX1176 | 46110 | SGAAGAASNDNT | 0.37 | 1.36 | 2.69 |
| 1472 | ATLVT012XX1028 | 45962 | NSTAAGSSNDNH | 1.8 | 1.18 | 0.46 |
| 1473 | ATLVT012XX2831 | 47765 | TGTTAAASNDNH | 1.38 | 1.22 | 1.14 |
| 1474 | ATLVT012XX2895 | 47829 | TSAAAGATNDNT | 0.92 | 1.66 | 1.36 |
| 1475 | ATLVT012XX27 | 44961 | NGAAGAASNDNT | 0.92 | 1.19 | 1.95 |
| 1476 | ATLVT012XX2720 | 47654 | TGQTGASTNDNH | 1.78 | 0.87 | 0.89 |
| 1477 | ATLVT012XX1653 | 46587 | SGTSAGSTNDNT | 0.89 | 0.88 | 2.4 |
| 1478 | ATLVT012XX1177 | 46111 | SGAAGAATNDNA | 0.48 | 1.56 | 2.22 |
| 1479 | ATLVT012XX677 | 45611 | NSATAAATNDNH | 2.11 | 1.22 | −0.13 |
| 1480 | ATLVT012XX502 | 45436 | NGTSAGSTNDNA | 1.16 | 0.74 | 2.1 |
| 1481 | ATLVT012XX499 | 45433 | NGTSAGSSNDNA | 0.93 | 1.4 | 1.65 |
| 1482 | ATLVT012XX133 | 45067 | NGATGGASNDNA | 2.44 | 0.31 | 0.47 |
| 1483 | ATLVT012XX795 | 45729 | NSESGAASNDNT | −0.13 | −0.43 | 5.81 |
| 1484 | ATLVT012XX2029 | 46963 | SSQAAGAINDNA | 0.44 | 1.71 | 2.07 |
| 1485 | ATLVT012XX2144 | 47078 | SSQTGASTNDNH | 0.29 | 2.39 | 1.45 |
| 1486 | ATLVT012XX3240 | 48174 | ISQSGAASNDNT | 0.48 | 1.43 | 2.35 |
| 1487 | ATLVT012XX1891 | 46825 | SSEAAGSTNDNA | 0.8 | 3.63 | −1.03 |
| 1488 | ATLVT012XX101 | 45035 | NGATAAAINDNH | −0.1 | 4.69 | −0.86 |
| 1489 | ATLVT012XX1866 | 46800 | SSATGGSSNDNT | 2.04 | 0.04 | 1.44 |
| 1490 | ATLVT012XX612 | 45546 | NSAAGASINDNT | 0.06 | 2.94 | 1.11 |
| 1491 | ATLVT012XX2805 | 47739 | TGTSAGSTNDNT | 2.46 | −0.02 | 0.81 |
| 1492 | ATLVT01XXX1218 | 46152 | SGASAGSSNDNT | 1.05 | 0.47 | 2.58 |
| 1493 | ATLVT012XX675 | 45609 | NSATAAASNDNT | 1.67 | −2.07 | 4.78 |
| 1494 | ATLVT012XX395 | 45329 | NGQTAASTNDNH | −0.53 | 4.01 | 0.72 |
| 1495 | ATLVT012XX2351 | 47285 | TGASAAASNDNH | 1.93 | −0.18 | 1.89 |
| 1496 | ATLVT012XX2145 | 47079 | SSQTGASTNDNT | 0.27 | 1.55 | 2.48 |
| 1497 | ATLVT012XX2988 | 47922 | TSATAGASNDNT | −0.01 | 1.28 | 3.32 |
| 1498 | ATLVT012XX2214 | 47148 | SSTSAASSNDNT | −1.31 | 3.33 | 2.91 |
| 1499 | ATLVT012XX2605 | 47539 | TGQAAGAINDNA | 0.28 | 1.61 | 2.39 |
| 1500 | ATLVT012XX1332 | 46266 | SGEAGGASNDNT | 1.84 | 0.29 | 1.41 |
| 1501 | ATLVT012XX25 | 44959 | NGAAGAASNDNA | 0.67 | 1.33 | 2.07 |
| 1502 | ATLVT012XX877 | 45811 | NSQAAGASNDNA | 0.42 | 1.7 | 2.02 |
| 1503 | ATLVT012XX1938 | 46872 | SSESAGSSNDNT | −1.63 | 1.42 | 5.89 |
| 1504 | ATLVT012XX1904 | 46838 | SSEAGASINDNH | 2.11 | 1.6 | −0.78 |
| 1505 | ATLVT012XX555 | 45489 | NGTTGAASNDNT | 2.05 | 0.59 | 0.63 |
| 1506 | ATLVT012XX2190 | 47124 | SSTAGASSNDNT | 0.76 | 0.69 | 2.69 |
| 1507 | ATLVT012XX2815 | 47749 | TGTSGASTNDNA | 0.05 | 3.51 | 0.28 |
| 1508 | ATLVT012XX2890 | 47824 | TSAAAGASNDNA | 0.28 | 2.18 | 1.59 |
| 1509 | ATLVT012XX761 | 45695 | NSEAGGATNDNH | −0.15 | 3.69 | 0.4 |
| 1510 | ATLVT012XX882 | 45816 | NSQAAGAINDNT | 1.24 | 1.13 | 1.3 |
| 1511 | ATLVT012XX2885 | 47819 | TSAAAASSNDNH | −1.7 | 5.44 | 0.78 |
| 1512 | ATLVT012XX1172 | 46106 | SGAAAGSINDNH | 0.69 | 1.4 | 1.87 |
| 1513 | ATLVT012XX2182 | 47116 | SSTAGAASNDNA | 1.71 | 0.53 | 1.24 |
| 1514 | ATLVT012XX2372 | 47306 | TGASAGSTNDNH | 0.52 | 1.64 | 1.85 |
| 1515 | ATLVT012XX3295 | 48229 | ISQTGASTNDNA | 0.18 | 3.96 | −0.55 |
| 1516 | ATLVT012XX2962 | 47896 | TSASGGASNDNA | 1.05 | 0.93 | 1.85 |
| 1517 | ATLVT012XX334 | 45268 | NGQAGGSINDNA | 1.48 | 0.89 | 1.16 |
| 1518 | ATLVT012XX1750 | 46684 | SSAAGAASNDNA | 0.29 | 1.12 | 2.9 |
| 1519 | ATLVT012XX236 | 45170 | NGESGGSSNDNH | 0.98 | 2.13 | 0.41 |
| 1520 | ATLVT012XX40 | 44974 | NGAAGGATNDNA | 0.24 | 1.51 | 2.47 |
| 1521 | ATLVT012XX2936 | 47870 | TSASAASTNDNH | −0.61 | 4.11 | 0.58 |
| 1522 | ATLVT012XX1693 | 46627 | SGTTAGATNDNA | 0.86 | 1.17 | 1.84 |
| 1523 | ATLVT012XX2422 | 47356 | TGATGAASNDNA | 0.75 | 1.37 | 1.77 |
| 1524 | ATLVT012XX736 | 45670 | NSEAAGAINDNA | 1.17 | 1.66 | 0.66 |
| 1525 | ATLVT012XX2135 | 47069 | SSQTGAASNDNH | 1.1 | −0.63 | 3.72 |
| 1526 | ATLVT012XX2405 | 47339 | TGATAASSNDNH | 0.57 | 1.5 | 1.89 |
| 1527 | ATLVT012XX3193 | 48127 | TSQAGAATNDNA | 0.53 | 1.59 | 1.84 |
| 1528 | ATLVT012XX1600 | 46534 | SGTAAGSSNDNA | 2.36 | 0.29 | 0.37 |
| 1529 | ATLVT012XX1503 | 46437 | SGQSAGATNDNT | 2.07 | 0.59 | 0.48 |
| 1530 | ATLVT012XX893 | 45827 | NSQAGAATNDNH | 1.32 | 1.21 | 0.96 |
| 1531 | ATLVT012XX2680 | 47614 | TGQSGGSSNDNA | 1.24 | 1.61 | 0.56 |
| 1532 | ATLVT012XX802 | 45736 | NSESGASTNDNA | 2.61 | 0.45 | −0.27 |
| 1533 | ATLVT012XX2280 | 47214 | SSTTGAASNDNT | 1.73 | 1.31 | 0.1 |
| 1534 | ATLVT012XX489 | 45423 | NGTSAASSNDNT | −0.69 | 0.59 | 5.19 |
| 1535 | ATLVT012XX2397 | 47331 | TGASGGSINDNT | 0.84 | 0.34 | 2.87 |
| 1536 | ATLVT012XX889 | 45823 | NSQAGAASNDNA | 0.99 | 1.18 | 1.54 |
| 1537 | ATLVT012XX26 | 44960 | NGAAGAASNDNH | 0.61 | 1.31 | 2.03 |
| 1538 | ATLVT012XX1518 | 46452 | SGQSGASSNDNT | 0.64 | 1.11 | 2.22 |
| 1539 | ATLVT012XX2283 | 47217 | SSTTGAATNDNT | 1.96 | 0.56 | 0.66 |
| 1540 | ATLVT012XX2951 | 47885 | TSASGAASNDNH | 1.06 | 0.8 | 1.89 |
| 1541 | ATLVT012XX2961 | 47895 | TSASGASTNDNT | 1.2 | −0.16 | 2.87 |
| 1542 | ATLVT012XX933 | 45867 | NSQSAGSSNDNT | 2.87 | 0.45 | −0.78 |
| 1543 | ATLVT012XX1939 | 46873 | SSESAGSTNDNA | −1.16 | 4.67 | 0.7 |
| 1544 | ATLVT012XX453 | 45387 | NGTAAGSSNDNT | 1.35 | 1.03 | 1.08 |
| 1545 | ATLVT012XX1787 | 46721 | SSASAGASNDNH | 0.78 | 2.14 | 0.62 |
| 1546 | ATLVT012XX2943 | 47877 | TSASAGATNDNT | −0.28 | 3.86 | 0.23 |
| 1547 | ATLVT012XX1662 | 46596 | SGTSGASSNDNT | 0.65 | 0.15 | 3.4 |
| 1548 | ATLVT012XX240 | 45174 | NGESGGSTNDNT | 2.05 | 1.68 | −0.99 |
| 1549 | ATLVT012XX1151 | 46085 | SGAAAAASNDNH | 0.92 | 1.08 | 1.71 |
| 1550 | ATLVT012XX1752 | 46686 | SSAAGAASNDNT | 0.92 | 1.18 | 1.58 |
| 1551 | ATLVT012XX2084 | 47018 | SSQSAGSTNDNH | 0.69 | 2.74 | −0.05 |
| 1552 | ATLVT012XX419 | 45353 | NGQTGASTNDNH | 0.38 | 0.23 | 3.71 |
| 1553 | ATLVT012XX1413 | 46347 | SGETAGSINDNT | 1.68 | 1.23 | 0.2 |
| 1554 | ATLVT012XX3371 | 48305 | TSTSAGASNDNH | −0.67 | 2.56 | 2.51 |
| 1555 | ATLVT012XX97 | 45031 | NGATAAASNDNA | 1.09 | 3.46 | −1.67 |
| 1556 | ATLVT012XX535 | 45469 | NGTTAASSNDNA | −0.44 | 3.28 | 1.18 |
| 1557 | ATLVT012XX1002 | 45936 | NSQTGGATNDNT | 1.03 | 1.07 | 1.49 |
| 1558 | ATLVT012XX51 | 44985 | NGASAAASNDNT | 0.26 | 0.83 | 3.13 |
| 1559 | ATLVT012XX534 | 45468 | NGTTAAATNDNT | 0.08 | 2.97 | 0.68 |
| 1560 | ATLVT012XX1976 | 46910 | SSETAASTNDNH | 0.15 | 0.6 | 3.61 |
| 1561 | ATLVT012XX159 | 45093 | NGEAAGASNDNT | 2.06 | 0.25 | 0.78 |
| 1562 | ATLVT012XX2956 | 47890 | TSASGASSNDNA | 0.21 | −0.62 | 5.06 |
| 1563 | ATLVT012XX2530 | 47464 | TGESGGASNDNA | −1.3 | 4.27 | 1.37 |
| 1564 | ATLVT012XX1765 | 46699 | SSAAGGATNDNA | 1.01 | 0.59 | 2.11 |
| 1565 | ATLVT012XX3341 | 48275 | TSTAGASSNDNH | 2.44 | 0.23 | 0.13 |
| 1566 | ATLVT012XX2217 | 47151 | SSTSAASINDNT | 1.7 | 1.72 | −0.51 |
| 1567 | ATLVT012XX3179 | 48113 | TSQAAGASNDNH | 0.41 | 1.41 | 2.09 |
| 1568 | ATLVT012XX3236 | 48170 | TSQSAGSTNDNH | −0.35 | 1.86 | 2.79 |
| 1569 | ATLVT012XX2034 | 46968 | SSQAAGSSNDNT | 0.53 | 1.06 | 2.31 |
| 1570 | ATLVT012XX3327 | 48261 | TSTAAGAINDNT | 0.03 | 2.87 | 0.85 |
| 1571 | ATLVT012XX3343 | 48277 | TSTAGASINDNA | 1.07 | 0.13 | 2.58 |
| 1572 | ATLVT012XX3185 | 48119 | TSQAAGSSNDNH | 0.62 | 1.32 | 1.8 |
| 1573 | ATLVT012XX3013 | 47947 | TSATGGATNDNA | 0.66 | 3.51 | −1.09 |
| 1574 | ATLVT012XX1608 | 46542 | SGTAGAASNDNT | 0.62 | 1.17 | 1.98 |
| 1575 | ATLVT012XX2319 | 47253 | TGAAAGAINDNT | 0.57 | 1.09 | 2.18 |
| 1576 | ATLVT012XX284 | 45218 | NGETGGSSNDNH | 2.01 | −0.56 | 1.82 |
| 1577 | ATLVT012XX492 | 45426 | NGTSAASINDNT | 3.53 | −1.32 | 0.19 |
| 1578 | ATLVT012XX326 | 45260 | NGQAGGASNDNH | 1.05 | 0.47 | 2.15 |
| 1579 | ATLVT012XX548 | 45482 | NGTTAGSSNDNH | 1.21 | 1.06 | 1.1 |
| 1580 | ATLVT012XX2721 | 47655 | TGQTGASTNDNT | 0.84 | 0.97 | 1.84 |
| 1581 | ATLVT012XX737 | 45671 | NSEAAGATNDNH | 0.53 | 1.25 | 2.02 |
| 1582 | ATLVT012XX3431 | 48365 | TSTTGAASNDNH | 1.42 | 0.65 | 1.25 |
| 1583 | ATLVT012XX1983 | 46917 | SSETAGAINDNT | −0.25 | 3.61 | 0.29 |
| 1584 | ATLVT012XX2949 | 47883 | TSASAGSTNDNT | 0.11 | 2.45 | 1.15 |
| 1585 | ATLVT012XX2912 | 47846 | TSAAGASINDNH | 0.55 | 1.73 | 1.32 |
| 1586 | ATLVT012XX2960 | 47894 | TSASGASINDNH | 0.19 | −0.1 | 4.28 |
| 1587 | ATLVT012XX2377 | 47311 | TGASGAATNDNA | 1.41 | 0.75 | 1.09 |
| 1588 | ATLVT012XX2171 | 47105 | SSTAAGASNDNH | −0.23 | 1.77 | 2.58 |
| 1589 | ATLVT012XX763 | 45697 | NSEAGGSSNDNA | 0.69 | 1.3 | 1.61 |
| 1590 | ATLVT012XX546 | 45480 | NGTTAGATNDNT | 1.06 | 0.77 | 1.64 |
| 1591 | ATLVT012XX1467 | 46401 | SGQAGAATNDNT | 0.32 | 0.34 | 3.47 |
| 1592 | ATLVT012XX2132 | 47066 | SSQTAGSTNDNH | 0.55 | 0.64 | 2.69 |
| 1593 | ATLVT012XX1411 | 46345 | SGETAGSTNDNA | 0.77 | 3.04 | −0.78 |
| 1594 | ATLVT012XX2602 | 47536 | TGQAAGASNDNA | 0.13 | 1.49 | 2.32 |
| 1595 | ATLVT012XX1365 | 46299 | SGESAGSTNDNT | 2 | 1.54 | −0.97 |
| 1596 | ATLVT012XX2844 | 47778 | TGTTAGASNDNT | 1.11 | 1.43 | 0.7 |
| 1597 | ATLVT012XX1405 | 46339 | SGETAGATNDNA | 2.19 | 1.12 | −0.75 |
| 1598 | ATLVT012XX2260 | 47194 | SSTTAASSNDNA | −0.25 | 4.18 | −0.5 |
| 1599 | ATLVT012XX3420 | 48354 | TSTTAGASNDNT | 1.41 | 0.89 | 0.86 |
| 1600 | ATLVT012XX1783 | 46717 | SSASAASTNDNA | 0.64 | 1.98 | 0.79 |
| 1601 | ATLVT012XX2309 | 47243 | TGAAAASSNDNH | 0.29 | 1.74 | 1.69 |
| 1602 | ATLVT012XX691 | 45625 | NSATAGSSNDNA | 1.09 | −0.02 | 2.58 |
| 1603 | ATLVT012XX1593 | 46527 | SGTAAASINDNT | 1.77 | −0.73 | 2.33 |
| 1604 | ATLVT012XX855 | 45789 | NSETGGASNDNT | 0.97 | 1.83 | 0.42 |
| 1605 | ATLVT012XX2443 | 47377 | TGATGGSTNDNA | −0.19 | 1.23 | 3.17 |
| 1606 | ATLVT012XX2310 | 47244 | TGAAAASSNDNT | −0.05 | 1.47 | 2.61 |
| 1607 | ATLVT012XX684 | 45618 | NSATAASTNDNT | 2.7 | −0.39 | 0.27 |
| 1608 | ATLVT012XX1994 | 46928 | SSETGAATNDNH | 1.68 | 0.29 | 1.15 |
| 1609 | ATLVT012XX563 | 45497 | NGTIGASINDNH | 0.57 | 1.44 | 1.57 |
| 1610 | ATLVT012XX2406 | 47340 | TGATAASSNDNT | −1.19 | 1.08 | 5.03 |
| 1611 | ATLVT012XX645 | 45579 | NSASAGSSNDNT | −0.03 | 2.22 | 1.59 |
| 1612 | ATLVT012XX1966 | 46900 | SSETAAASNDNA | 1.65 | −0.07 | 1.64 |
| 1613 | ATLVT012XX3262 | 48196 | TSQTAAASNDNA | 2.83 | −0.48 | 0.15 |
| 1614 | ATLVT012XX363 | 45297 | NGQSGAASNDNT | 1.62 | 0.88 | 0.47 |
| 1615 | ATLVT012XX2317 | 47251 | TGAAAGAINDNA | 0.44 | 1.46 | 1.73 |
| 1616 | ATLVT012XX2847 | 47781 | TGTTAGATNDNT | 1.33 | 0.59 | 1.32 |
| 1617 | ATLVT012XX3344 | 48278 | TSTAGASINDNH | 1.3 | 0.85 | 1.05 |
| 1618 | ATLVT012XX75 | 45009 | NGASGAASNDNT | 0.71 | 1.68 | 0.98 |
| 1619 | ATLVT012XX3282 | 48216 | TSQTAGSSNDNT | 1.22 | 0.6 | 1.49 |
| 1620 | ATLVT012XX221 | 45155 | NGESGAATNDNH | 1.04 | 0.95 | 1.37 |
| 1621 | ATLVT012XX2755 | 47689 | TGTAAGSINDNA | 2.4 | −0.29 | 0.62 |
| 1622 | ATLVT012XX1745 | 46679 | SSAAAGSSNDNH | 0.03 | 2.13 | 1.56 |
| 1623 | ATLVT012XX2736 | 47670 | TGTAAAASNDNT | −1.53 | 4.97 | 0.58 |
| 1624 | ATLVT012XX493 | 45427 | NGTSAGASNDNA | 0.89 | 1.77 | 0.53 |
| 1625 | ATLVT012XX1770 | 46704 | SSAAGGSSNDNT | 0.48 | 1.85 | 1.12 |
| 1626 | ATLVT012XX1551 | 46485 | SGQTAGATNDNT | 0.35 | 2.61 | 0.37 |
| 1627 | ATLVT012XX3372 | 48306 | TSTSAGASNDNT | 0.42 | 1.21 | 2.03 |
| 1628 | ATLVT012XX2141 | 47075 | SSQTGASSNDNH | −1.06 | 2.3 | 3.17 |
| 1629 | ATLVT012XX1689 | 46623 | SGTTAASINDNT | 0.73 | 0.11 | 2.89 |
| 1630 | ATLVT012XX467 | 45401 | NGTAGASINDNH | 1.5 | 0.36 | 1.26 |
| 1631 | ATLVT012XX42 | 44976 | NGAAGGATNDNT | −0.18 | 1.95 | 2.09 |
| 1632 | ATLVT012XX491 | 45425 | NGTSAASTNDNH | 1.03 | 1.79 | 0.22 |
| 1633 | ATLVT012XX1806 | 46740 | SSASGASSNDNT | −1 | 2.21 | 3.15 |
| 1634 | ATLVT012XX406 | 45340 | NGQTAGSTNDNA | 2.59 | 0.44 | −0.74 |
| 1635 | ATLVT012XX487 | 45421 | NGTSAASSNDNA | −0.58 | 2.21 | 2.42 |
| 1636 | ATLVT012XX1651 | 46585 | SGTSAGSTNDNA | 1.6 | 0.15 | 1.32 |
| 1637 | ATLVT012XX1936 | 46870 | SSESAGSSNDNA | −1.13 | 3.1 | 2.2 |
| 1638 | ATLVT012XX1569 | 46503 | SGQTGASTNDNT | 1.49 | 0.26 | 1.36 |
| 1639 | ATLVT012XX173 | 45107 | NGEAGAATNDNH | 0.16 | 2.99 | 0.12 |
| 1640 | ATLVT012XX687 | 45621 | NSATAGASNDNT | 0.85 | 0.3 | 2.4 |
| 1641 | ATLVT012XX2413 | 47347 | TGATAGATNDNA | 1.67 | −0.19 | 1.63 |
| 1642 | ATLVT012XX1171 | 46105 | SGAAAGSINDNA | 0.58 | 1.66 | 1.11 |
| 1643 | ATLVT012XX2140 | 47074 | SSQTGASSNDNA | −0.1 | −0.09 | 4.51 |
| 1644 | ATLVT012XX1068 | 46002 | NSTSAASINDNT | 1.13 | 0.37 | 1.82 |
| 1645 | ATLVT012XX2753 | 47687 | TGTAAGSSNDNH | 1.46 | 1.1 | 0.31 |
| 1646 | ATLVT012XX2347 | 47281 | TGAAGGSTNDNA | 0.17 | 1.73 | 2.3 |
| 1647 | ATLVT012XX373 | 45307 | NGQSGGASNDNA | 0.34 | 1.32 | 1.93 |
| 1648 | ATLVT012XX428 | 45362 | NGQTGGSSNDNH | 1.03 | 1.97 | −0.11 |
| 1649 | ATLVT012XX71 | 45005 | NGASAGSTNDNH | 1.6 | 0.43 | 0.9 |
| 1650 | ATLVT012XX858 | 45792 | NSETGGATNDNT | 0.46 | 2.08 | 0.72 |
| 1651 | ATLVT012XX2952 | 47886 | TSASGAASNDNT | 0.99 | 0.96 | 1.24 |
| 1652 | ATLVT012XX116 | 45050 | NGATAGSSNDNH | 1.91 | 0.74 | −0.04 |
| 1653 | ATLVT012XX3325 | 48259 | TSTAAGAINDNA | −0.81 | 4 | 0.43 |
| 1654 | ATLVT012XX1453 | 46387 | SGQAAGAINDNA | 0.02 | 1.57 | 2.12 |
| 1655 | ATLVT012XX2913 | 47847 | TSAAGASTNDNT | 0.23 | 1.58 | 1.74 |
| 1656 | ATLVT012XX19 | 44953 | NGAAAGSSNDNA | 0.13 | 2 | 1.37 |
| 1657 | ATLVT012XX1875 | 46809 | SSEAAAATNDNT | −1.6 | 2.45 | 3.75 |
| 1658 | ATLVT012XX1951 | 46885 | SSESGASINDNA | −0.78 | 4.44 | −0.2 |
| 1659 | ATLVT012XX2593 | 47527 | TGQAAAATNDNA | 0.68 | −0.69 | 3.86 |
| 1660 | ATLVT012XX353 | 45287 | NGQSAGAINDNH | 0.84 | 1.35 | 0.97 |
| 1661 | ATLVT012XX464 | 45398 | NGTAGASSNDNH | 0.32 | 1.41 | 1.78 |
| 1662 | ATLVT012XX3258 | 48192 | TSQSGGSSNDNT | 0.08 | 1.1 | 2.58 |
| 1663 | ATLVT012XX699 | 45633 | NSATGAASNDNT | −0.7 | 1.63 | 3.23 |
| 1664 | ATLVT012XX2070 | 47004 | SSQSAASSNDNT | 1.55 | −1.26 | 3.09 |
| 1665 | ATLVT012XX2842 | 47776 | TGTTAGASNDNA | 0.37 | 1.92 | 1.03 |
| 1666 | ATLVT012XX1804 | 46738 | SSASGASSNDNA | 0.92 | 1.07 | 1.17 |
| 1667 | ATLVT012XX2164 | 47098 | SSTAAASSNDNA | 3.1 | −0.34 | −0.77 |
| 1668 | ATLVT012XX2265 | 47199 | SSTTAASTNDNT | −0.53 | 3.3 | 0.79 |
| 1669 | ATLVT012XX2517 | 47451 | TGESAGSTNDNT | 0.42 | 0.64 | 2.55 |
| 1670 | ATLVT012XX1952 | 46886 | SSESGASTNDNH | −0.02 | 1.33 | 2.42 |
| 1671 | ATLVT012XX1799 | 46733 | SSASGAASNDNH | 1.48 | 0.89 | 0.41 |
| 1672 | ATLVT012XX593 | 45527 | NSAAAGATNDNH | 1.05 | 1.21 | 0.74 |
| 1673 | ATLVT012XX1710 | 46644 | SGTTGASSNDNT | 1.69 | −0.71 | 2.09 |
| 1674 | ATLVT012XX1490 | 46424 | SGQSAAATNDNH | 2.18 | 1.06 | −1.04 |
| 1675 | ATLVT012XX2225 | 47159 | SSTSAGSSNDNH | 0.98 | 1.51 | 0.43 |
| 1676 | ATLVT012XX2975 | 47909 | TSATAAASNDNH | 0.22 | 3.04 | −0.25 |
| 1677 | ATLVT012XX3186 | 48120 | TSQAAGSSNDNT | 0.08 | 0.98 | 2.65 |
| 1678 | ATLVT012XX1471 | 46405 | SGQAGASINDNA | −1.02 | 1.53 | 3.82 |
| 1679 | ATLVT012XX3012 | 47946 | TSATGGASNDNT | 1.99 | 0.62 | −0.16 |
| 1680 | ATLVT012XX2026 | 46960 | SSQAAGASNDNA | 0.23 | 1.76 | 1.38 |
| 1681 | ATLVT012XX892 | 45826 | NSQAGAATNDNA | −0.72 | 3.52 | 0.74 |
| 1682 | ATLVT012XX328 | 45262 | NGQAGGATNDNA | 1.57 | −0.21 | 1.61 |
| 1683 | ATLVT012XX2376 | 47310 | TGASGAASNDNT | 1.59 | 0.6 | 0.53 |
| 1684 | ATLVT012XX2332 | 47266 | TGAAGASSNDNA | 0.03 | 1.4 | 2.17 |
| 1685 | ATLVT012XX1769 | 46703 | SSAAGGSSNDNH | 1.36 | 0.66 | 0.84 |
| 1686 | ATLVT012XX3202 | 48136 | TSQAGGASNDNA | 0.27 | 0.65 | 2.7 |
| 1687 | ATLVT012XX3433 | 48367 | TSTTGAATNDNA | 0.77 | 0.62 | 1.88 |
| 1688 | ATLVT012XX2504 | 47438 | TGESAASTNDNH | 0.36 | 1.96 | 0.85 |
| 1689 | ATLVT012XX1612 | 46546 | SGTAGASSNDNA | 0.24 | 0.94 | 2.36 |
| 1690 | ATLVT012XX2578 | 47512 | TGETGGASNDNA | 1.79 | 0.73 | −0.02 |
| 1691 | ATLVT012XX3328 | 48262 | TSTAAGSSNDNA | 1.18 | 1.26 | 0.33 |
| 1692 | ATLVT012XX3039 | 47973 | TSEAAGAINDNT | 0.34 | 0.89 | 2.25 |
| 1693 | ATLVT012XX2935 | 47869 | TSASAASTNDNA | 0.12 | 2.38 | 0.71 |
| 1694 | ATLVT012XX1803 | 46737 | SSASGAATNDNT | 1.09 | 0.43 | 1.54 |
| 1695 | ATLVT012XX1107 | 46041 | NSTTAAATNDNI | −0.41 | 1.84 | 2.29 |
| 1696 | ATLVT012XX2378 | 47312 | TGASGAATNDNH | 1.66 | 0.44 | 0.55 |
| 1697 | ATLVT012XX2990 | 47924 | TSATAGAINDNH | 1.06 | 1.08 | 0.76 |
| 1698 | ATLVT012XX460 | 45394 | NGTAGAATNDNA | 0.72 | 0.62 | 1.93 |
| 1699 | ATLVT012XX900 | 45834 | NSQAGASINDNT | −0.38 | 0.52 | 3.95 |
| 1700 | ATLVT012XX79 | 45013 | NGASGASSNDNA | 2.31 | −0.66 | 0.84 |
| 1701 | ATLVT012XX3378 | 48312 | TSTSAGSSNDNT | −1.15 | 2.2 | 3.09 |
| 1702 | ATLVT012XX2902 | 47836 | TSAAGAASNDNA | 0.17 | 1.28 | 2.02 |
| 1703 | ATLVT012XX39 | 44973 | NGAAGGASNDNT | 0.31 | 1.07 | 2.05 |
| 1704 | ATLVT012XX986 | 45920 | NSQTGAASNDNH | 1.18 | 0.35 | 1.48 |
| 1705 | ATLVT012XX1208 | 46142 | SGASAASTNDNH | 0.95 | 1.82 | −0.01 |
| 1706 | ATLVT012XX1807 | 46741 | SSASGASTNDNA | 0.95 | 0.99 | 1.03 |
| 1707 | ATLVT012XX2041 | 46975 | SSQAGAATNDNA | 0.22 | 0.89 | 2.41 |
| 1708 | ATLVT012XX2306 | 47240 | TGAAAAATNDNH | 0.65 | 0.98 | 1.56 |
| 1709 | ATLVT012XX461 | 45395 | NGTAGAATNDNH | 1.14 | 0.41 | 1.44 |
| 1710 | ATLVT012XX560 | 45494 | NGTTGASSNDNH | −0.22 | 2.03 | 1.68 |
| 1711 | ATLVT012XX1880 | 46814 | SSEAAASINDNH | 2.54 | 0.29 | −0.8 |
| 1712 | ATLVT012XX3331 | 48265 | TSTAAGSINDNA | 0.77 | 1.5 | 0.67 |
| 1713 | ATLVT012XX2539 | 47473 | TGESGGSTNDNA | −0.82 | 1.78 | 3.03 |
| 1714 | ATLVT012XX3036 | 47970 | TSEAAGASNDNT | 0.13 | 1.15 | 2.2 |
| 1715 | ATLVT012XX1645 | 46579 | SGTSAGATNDNA | 1.32 | 0.48 | 1.01 |
| 1716 | ATLVT012XX2356 | 47290 | TGASAASSNDNA | −1.1 | 4.25 | 0.32 |
| 1717 | ATLVT012XX2916 | 47850 | ISAAGGASNDNT | −0.38 | 1.82 | 2.19 |
| 1718 | ATLVT012XX833 | 45767 | NSETAGAINDNH | 1.26 | 0.89 | 0.58 |
| 1719 | ATLVT012XX2925 | 47859 | TSAAGGSINDNT | 0.15 | 1.87 | 1.21 |
| 1720 | ATLVT012XX188 | 45122 | NGEAGGSSNDNH | 3.3 | −0.23 | −1.47 |
| 1721 | ATLVT012XX48 | 44982 | NGAAGGSINDNT | −0.06 | 1.66 | 1.85 |
| 1722 | ATLVT012XX2967 | 47901 | TSASGGATNDNT | 0.27 | 0.75 | 2.45 |
| 1723 | ATLVT012XX564 | 45498 | NGTTGASINDNT | 1.42 | 0.64 | 0.61 |
| 1724 | ATLVT012XX1500 | 46434 | SGQSAGASNDNT | 1.83 | 1.01 | −0.58 |
| 1725 | ATLVT012XX1265 | 46199 | SGATAGSSNDNH | 2.84 | −0.25 | −0.7 |
| 1726 | ATLVT012XX2316 | 47250 | TGAAAGASNDNT | 0.51 | 0.98 | 1.72 |
| 1727 | ATLVT012XX842 | 45776 | NSETGAASNDNH | 0.15 | −0.09 | 3.7 |
| 1728 | ATLVT012XX136 | 45070 | NGATGGATNDNA | 1.05 | 0.27 | 1.7 |
| 1729 | ATLVT012XX539 | 45473 | NGTTAASINDNH | −0.65 | 4.49 | −0.82 |
| 1730 | ATLVT012XX644 | 45578 | NSASAGSSNDNH | 1.06 | 0.84 | 0.91 |
| 1731 | ATLVT012XX2516 | 47450 | TGESAGSTNDNH | 0.59 | 2.03 | 0.19 |
| 1732 | ATLVT012XX1649 | 46583 | SGTSAGSSNDNH | 1.92 | 1.27 | −1.13 |
| 1733 | ATLVT012XX2264 | 47198 | SSTTAASTNDNH | 0.25 | 3.04 | −0.54 |
| 1734 | ATLVT012XX1896 | 46830 | SSEAGAASNDNT | 1.18 | 0.14 | 1.58 |
| 1735 | ATLVT012XX3438 | 48372 | TSTTGASSNDNI | 0.64 | 0.73 | 1.74 |
| 1736 | ATLVT012XX3377 | 48311 | TSTSAGSSNDNH | −0.07 | 1.44 | 2.03 |
| 1737 | ATLVT012XX1606 | 46540 | SGTAGAASNDNA | 0.42 | 0.1 | 2.91 |
| 1738 | ATLVT012XX561 | 45495 | NGTTGASSNDNT | 1.03 | 0.69 | 1.12 |
| 1739 | ATLVT012XX2865 | 47799 | TGTTGASTNDNT | 0.43 | 1.76 | 0.76 |
| 1740 | ATLVT012XX2761 | 47695 | TGTAGAATNDNA | −0.08 | 0.92 | 2.72 |
| 1741 | ATLVT012XX2094 | 47028 | SSQSGASSNDNT | 1.12 | −0.19 | 2.08 |
| 1742 | ATLVT012XX1104 | 46038 | NSTTAAASNDNT | 0.95 | 0.61 | 1.35 |
| 1743 | ATLVT012XX1648 | 46582 | SGTSAGSSNDNA | 0.69 | 0.76 | 1.59 |
| 1744 | ATLVT012XX2791 | 47725 | TGTSAASTNDNA | 0.59 | 1.96 | 0.22 |
| 1745 | ATLVT012XX2806 | 47740 | TGTSGAASNDNA | 0.97 | 0.94 | 0.86 |
| 1746 | ATLVT012XX1152 | 46086 | SGAAAAASNDNT | 0.66 | 0.67 | 1.76 |
| 1747 | ATLVT012XX3106 | 48040 | TSESGGASNDNA | 0.68 | −0.06 | 2.65 |
| 1748 | ATLVT012XX3287 | 48221 | TSQTGAASNDNH | 0.33 | 1.48 | 1.27 |
| 1749 | ATLVT012XX878 | 45812 | NSQAAGASNDNH | 0.43 | 1.05 | 1.65 |
| 1750 | ATLVT012XX1459 | 46393 | SGQAAGSINDNA | 0.45 | 1.7 | 0.77 |
| 1751 | ATLVT012XX885 | 45819 | NSQAAGSSNDNT | 0.16 | 1.44 | 1.6 |
| 1752 | ATLVT012XX3437 | 48371 | TSTTGASSNDNH | 1.35 | 0.44 | 0.84 |
| 1753 | ATLVT012XX1585 | 46519 | SGTAAAATNDNA | −1.39 | −0.54 | 6.78 |
| 1754 | ATLVT012XX36 | 44970 | NGAAGASTNDNT | 1.76 | −0.04 | 0.74 |
| 1755 | ATLVT012XX498 | 45432 | NGTSAGATNDNT | 0.45 | 1.16 | 1.46 |
| 1756 | ATLVT012XX3413 | 48347 | TSTTAASSNDNH | −0.55 | 0.54 | 3.96 |
| 1757 | ATLVT012XX316 | 45250 | NGQAGAATNDNA | 1.62 | 0.11 | 0.78 |
| 1758 | ATLVT012XX1246 | 46180 | SGATAAASNDNA | 2.11 | 0.78 | −0.92 |
| 1759 | ATLVT012XX2682 | 47616 | TGQSGGSSNDNT | 1.04 | 1.24 | 0.31 |
| 1760 | ATLVT012XX1679 | 46613 | SGTTAAASNDNH | 1.11 | 0.91 | 0.63 |
| 1761 | ATLVT012XX2958 | 47892 | TSASGASSNDNT | 0.55 | 0.11 | 2.59 |
| 1762 | ATLVT012XX2613 | 47547 | TGQAAGSINDNT | 0.22 | 0.57 | 2.57 |
| 1763 | ATLVT012XX281 | 45215 | NGETGGATNDNH | −1.04 | 3.4 | 1.07 |
| 1764 | ATLVT012XX1441 | 46375 | SGQAAAATNDNA | −0.65 | −0.7 | 5.67 |
| 1765 | ATLVT012XX3332 | 48266 | TSTAAGSTNDNH | 0.37 | 1.98 | 0.47 |
| 1766 | ATLVT012XX3149 | 48083 | TSETGASSNDNH | 2.94 | −0.54 | −0.7 |
| 1767 | ATLVT012XX324 | 45258 | NGQAGASINDNT | 2.21 | −1.01 | 1.16 |
| 1768 | ATLVT012XX1544 | 46478 | SGQTAASTNDNH | 1.21 | 1.77 | −0.71 |
| 1769 | ATLVT012XX2337 | 47271 | TGAAGASTNDNT | 0.23 | 1.1 | 1.83 |
| 1770 | ATLVT012XX2043 | 46977 | SSQAGAATNDNT | 0.65 | −0.12 | 2.66 |
| 1771 | ATLVT012XX640 | 45574 | NSASAGAINDNA | 0.66 | 1.8 | 0.18 |
| 1772 | ATLVT012XX1404 | 46338 | SGETAGASNDNT | 0.45 | 2.3 | −0.12 |
| 1773 | ATLVT012XX1801 | 46735 | SSASGAATNDNA | 1.21 | 0.55 | 0.83 |
| 1774 | ATLVT012XX3276 | 48210 | TSQTAGASNDNT | 0.87 | 0.61 | 1.33 |
| 1775 | ATLVT012XX647 | 45581 | NSASAGSINDNH | 0.81 | 1.24 | 0.63 |
| 1776 | ATLVT012XX295 | 45229 | NGQAAASSNDNA | 3.23 | −1.3 | −0.25 |
| 1777 | ATLVT012XX3067 | 48001 | TSEAGGSINDNA | 1.38 | −0.18 | 1.48 |
| 1778 | ATLVT012XX3421 | 48355 | TSTTAGAINDNA | 1.24 | 0.64 | 0.66 |
| 1779 | ATLVT012XX2615 | 47549 | TGQAGAASNDNH | −0.09 | 1.55 | 1.76 |
| 1780 | ATLVT012XX1886 | 46820 | SSEAAGAINDNH | −0.21 | 1.78 | 1.68 |
| 1781 | ATLVT012XX703 | 45637 | NSATGASSNDNA | 1.96 | 0.88 | −0.9 |
| 1782 | ATLVT012XX2075 | 47009 | SSQSAGASNDNH | 0.18 | 1.13 | 1.82 |
| 1783 | ATLVT01XXX78 | 45012 | NGASGAATNDNT | 0.7 | 0.5 | 1.75 |
| 1784 | ATLVT012XX1554 | 46488 | SGQTAGSSNDNT | 0.11 | 2.36 | 0.37 |
| 1785 | ATLVT012XX442 | 45376 | NGTAAASTNDNA | −1.4 | 4.06 | 0.74 |
| 1786 | ATLVT012XX304 | 45238 | NGQAAGATNDNA | 0.89 | 0.78 | 1.04 |
| 1787 | ATLVT012XX1840 | 46774 | SSATAGSSNDNA | 3.7 | −1.29 | −1.12 |
| 1788 | ATLVT012XX1702 | 46636 | SGTTGAASNDNA | 1.69 | 0.19 | 0.43 |
| 1789 | ATLVT012XX3115 | 48049 | TSESGGSINDNA | 1.06 | 0.33 | 1.32 |
| 1790 | ATLVT012XX3424 | 48358 | TSTTAGSSNDNA | 1.28 | 0.34 | 0.92 |
| 1791 | ATLVT012XX1013 | 45947 | NSTAAAATNDNH | 1.72 | 0.93 | −0.61 |
| 1792 | ATLVT012XX176 | 45110 | NGEAGASSNDNH | −0.98 | 5.03 | −1.25 |
| 1793 | ATLVT012XX1757 | 46691 | SSAAGASSNDNH | −0.2 | 2.13 | 1.15 |
| 1794 | ATLVT012XX1458 | 46392 | SGQAAGSSNDNT | 0.05 | 0.93 | 2.25 |
| 1795 | ATLVT012XX132 | 45066 | NGATGASTNDNT | 1.19 | 1.04 | 0.14 |
| 1796 | ATLVT012XX3016 | 47950 | TSATGGSSNDNA | 1.99 | 0.38 | −0.38 |
| 1797 | ATLVT012XX1105 | 46039 | NSTTAAATNDNA | 0.08 | 2.28 | 0.46 |
| 1798 | ATLVT012XX232 | 45166 | NGESGGATNDNA | 1.55 | 1.16 | −0.63 |
| 1799 | ATLVT012XX247 | 45181 | NGETAASSNDNA | −0.09 | 2.99 | −0.17 |
| 1800 | ATLVT012XX2580 | 47514 | TGETGGASNDNT | 1.55 | 0.73 | −0.07 |
| 1801 | ATLVT012XX3379 | 48313 | TSTSAGSTNDNA | −0.6 | 0.84 | 3.46 |
| 1802 | ATLVT012XX367 | 45301 | NGQSGASSNDNA | 1.99 | −0.35 | 0.54 |
| 1803 | ATLVT012XX849 | 45783 | NSETGASSNDNT | 2.54 | −0.05 | −0.79 |
| 1804 | ATLVT012XX3390 | 48324 | TSTSGASSNDNT | −0.75 | 2.78 | 1.21 |
| 1805 | ATLVT012XX2083 | 47017 | SSQSAGSINDNA | −1.18 | 5 | −0.91 |
| 1806 | ATLVT012XX2945 | 47879 | TSASAGSSNDNH | 1.69 | 0.64 | −0.22 |
| 1807 | ATLVT012XX1895 | 46829 | SSEAGAASNDNH | 1.19 | −0.29 | 1.81 |
| 1808 | ATLVT012XX307 | 45241 | NGQAAGSSNDNA | 1.28 | 0.28 | 0.93 |
| 1809 | ATLVT012XX2180 | 47114 | SSTAAGSINDNH | 0.7 | 0.8 | 1.25 |
| 1810 | ATLVT012XX2953 | 47887 | ISASGAATNDNA | 0.41 | 0.93 | 1.57 |
| 1811 | ATLVT012XX890 | 45824 | NSQAGAASNDNH | 1.16 | 0.59 | 0.73 |
| 1812 | ATLVT012XX23 | 44957 | NGAAAGSTNDNH | 1.08 | 0.37 | 1.13 |
| 1813 | ATLVT012XX2367 | 47301 | TGASAGAINDNT | −0.95 | 2.93 | 1.34 |
| 1814 | ATLVT012XX210 | 45144 | NGESAGATNDNT | −1.46 | −0.43 | 6.52 |
| 1815 | ATLVT012XX3051 | 47985 | TSEAGAATNDNT | 0.52 | 0.77 | 1.58 |
| 1816 | ATLVT012XX3334 | 48268 | TSTAGAASNDNA | 0.99 | 0.52 | 1.09 |
| 1817 | ATLVT012XX1210 | 46144 | SGASAGASNDNA | 0.84 | 1.31 | 0.33 |
| 1818 | ATLVT012XX1407 | 46341 | SGETAGAINDNT | 0.99 | 1.12 | 0.31 |
| 1819 | ATLVT012XX1515 | 46449 | SGQSGAATNDNT | 0.27 | 0.43 | 2.42 |
| 1820 | ATLVT012XX1831 | 46765 | SSATAASTNDNA | 2.91 | −1.14 | −0.07 |
| 1821 | ATLVT012XX350 | 45284 | NGQSAGASNDNH | 0.65 | 0.22 | 2.04 |
| 1822 | ATLVT012XX2313 | 47247 | TGAAAASINDNT | −0.2 | 1.57 | 1.76 |
| 1823 | ATLVT012XX369 | 45303 | NGQSGASSNDNT | 0.19 | −0.98 | 4.37 |
| 1824 | ATLVT012XX1962 | 46896 | SSESGGSSNDNT | 0.86 | 0.87 | 0.82 |
| 1825 | ATLVT012XX3277 | 48211 | TSQTAGATNDNA | 1.41 | 0.12 | 0.83 |
| 1826 | ATLVT012XX2665 | 47599 | TGQSGAATNDNA | −0.13 | 1.58 | 1.61 |
| 1827 | ATLVT012XX1609 | 46543 | SGTAGAATNDNA | −1.05 | 0.62 | 4.42 |
| 1828 | ATLVT012XX2324 | 47258 | TGAAAGSINDNH | 0.14 | 1.14 | 1.7 |
| 1829 | ATLVT012XX1824 | 46758 | SSATAAASNDNT | 2.41 | −0.44 | −0.17 |
| 1830 | ATLVT012XX2383 | 47317 | TGASGASINDNA | −0.2 | 2.83 | 0.11 |
| 1831 | ATLVT012XX1781 | 46715 | SSASAASSNDNH | −0.64 | 0.62 | 3.68 |
| 1832 | ATLVT012XX1561 | 46495 | SGQTGAATNDNA | 0.15 | 0.49 | 2.5 |
| 1833 | ATLVT012XX1995 | 46929 | SSETGAATNDNT | 1.18 | −0.27 | 1.71 |
| 1834 | ATLVT012XX192 | 45126 | NGEAGGSINDNT | 1.42 | 1.52 | −1 |
| 1835 | ATLVT012XX1346 | 46280 | SGESAAATNDNH | 1.82 | 0.51 | −0.4 |
| 1836 | ATLVT012XX3057 | 47991 | TSEAGASINDNT | 0.68 | 1.53 | 0.24 |
| 1837 | ATLVT012XX2747 | 47681 | TGTAAGASNDNH | 0.05 | 1.65 | 1.16 |
| 1838 | ATLVT012XX1449 | 46383 | SGQAAASINDNT | 2.08 | −0.19 | 0.04 |
| 1839 | ATLVT012XX3207 | 48141 | TSQAGGAINDNT | 0.24 | 0.61 | 2.16 |
| 1840 | ATLVT012XX1564 | 46498 | SGQTGASSNDNA | 0.53 | 1.11 | 1.02 |
| 1841 | ATLVT012XX3337 | 48271 | TSTAGAATNDNA | 0.69 | 0.73 | 1.23 |
| 1842 | ATLVT012XX1751 | 46685 | SSAAGAASNDNH | 0.18 | 0.89 | 1.9 |
| 1843 | ATLVT012XX3429 | 48363 | TSTTAGSINDNT | −0.62 | 1.54 | 2.43 |
| 1844 | ATLVT012XX2625 | 47559 | TGQAGASINDNT | −1.26 | −0.5 | 6.16 |
| 1845 | ATLVT012XX1746 | 46680 | SSAAAGSSNDNT | −0.77 | −0.35 | 5.12 |
| 1846 | ATLVT012XX1421 | 46355 | SGETGASSNDNH | −0.53 | 3.58 | −0.36 |
| 1847 | ATLVT012XX490 | 45424 | NGTSAASINDNA | 0.83 | 0.63 | 1.11 |
| 1848 | ATLVT012XX981 | 45915 | NSQTAGSSNDNI | 1.91 | −0.13 | 0.23 |
| 1849 | ATLVT012XX2454 | 47388 | TGEAAASSNDNT | 1.46 | 0.92 | −0.36 |
| 1850 | ATLVT012XX3175 | 48109 | TSQAAASTNDNA | 2.05 | −0.45 | 0.41 |
| 1851 | ATLVT012XX3281 | 48215 | TSQTAGSSNDNH | 0.3 | 1.63 | 0.71 |
| 1852 | ATLVT012XX1586 | 46520 | SGTAAAATNDNH | 0.27 | 0.91 | 1.68 |
| 1853 | ATLVT012XX658 | 45592 | NSASGASINDNA | 1 | 0.94 | 0.39 |
| 1854 | ATLVT012XX310 | 45244 | NGQAAGSINDNA | 2.14 | −0.27 | −0.02 |
| 1855 | ATLVT012XX2628 | 47562 | TGQAGGASNDNT | 0.33 | 0.98 | 1.49 |
| 1856 | ATLVT012XX1764 | 46698 | SSAAGGASNDNT | 0.27 | 0.34 | 2.4 |
| 1857 | ATLVT012XX2331 | 47265 | TGAAGAATNDNT | 0 | 1.13 | 1.86 |
| 1858 | ATLVT012XX3336 | 48270 | TSTAGAASNDNT | 0.85 | 0.74 | 0.89 |
| 1859 | ATLVT012XX944 | 45878 | NSQSGASSNDNH | 0.56 | 1.78 | 0.05 |
| 1860 | ATLVT012XX2437 | 47371 | TGATGGATNDNA | −0.13 | 0.5 | 2.86 |
| 1861 | ATLVT012XX1010 | 45944 | NSTAAAASNDNH | −0.46 | 2.63 | 0.7 |
| 1862 | ATLVT012XX505 | 45439 | NGTSGAASNDNA | 0.37 | 1 | 1.37 |
| 1863 | ATLVT012XX1457 | 46391 | SGQAAGSSNDNH | 0.55 | 1.78 | 0.06 |
| 1864 | ATLVT012XX2589 | 47523 | TGETGGSINDNI | 2.02 | −0.13 | −0.01 |
| 1865 | ATLVT012XX481 | 45415 | NGTSAAASNDNA | 1.21 | 0.15 | 1.02 |
| 1866 | ATLVT012XX2369 | 47303 | TGASAGSSNDNH | 0.24 | 1.33 | 1.16 |
| 1867 | ATLVT012XX596 | 45530 | NSAAAGSSNDNH | 0.84 | 1.28 | 0.19 |
| 1868 | ATLVT012XX2664 | 47598 | TGQSGAASNDNT | 0.2 | 1.73 | 0.7 |
| 1869 | ATLVT012XX262 | 45196 | NGETAGSTNDNA | −1.14 | 5.04 | −1.27 |
| 1870 | ATLVT012XX1424 | 16358 | SGETGASTNDNH | 0.89 | 0.69 | 0.84 |
| 1871 | ATLVT012XX3111 | 48045 | TSESGGAINDNT | 0.46 | 0.53 | 1.77 |
| 1872 | ATLVT012XX1219 | 46153 | SGASAGSTNDNA | 0.75 | −0.55 | 2.66 |
| 1873 | ATLVT012XX1347 | 46281 | SGESAAATNDNT | 3.47 | −1.38 | −0.94 |
| 1874 | ATLVT012XX2632 | 47566 | TGQAGGSSNDNA | 0.37 | 0.57 | 1.87 |
| 1875 | ATLVT012XX2027 | 46961 | SSQAAGASNDNH | −0.29 | 1.32 | 2.01 |
| 1876 | ATLVT012XX2411 | 47345 | TGATAGASNQNH | 2.17 | −0.83 | 0.56 |
| 1877 | ATLVT012XX3060 | 47994 | TSEAGGASNDNT | −0.45 | 1.68 | 1.81 |
| 1878 | ATLVT012XX287 | 45221 | NGETGGSINDNH | 0.28 | 1.6 | 0.66 |
| 1879 | ATLVT012XX2546 | 47480 | TGETAAATNDNH | 2.73 | −0.62 | −0.69 |
| 1880 | ATLVT012XX683 | 45617 | NSATAASTNDNH | −0.26 | 3.32 | −0.64 |
| 1881 | ATLVT012XX1688 | 46622 | SGTTAASINDNH | 0.71 | 1.38 | 0.2 |
| 1882 | ATLVT012XX2210 | 47144 | SSTSAAATNDNH | 2.12 | 0.54 | −1.16 |
| 1883 | ATLVT012XX2121 | 47055 | SSQTAASTNDNT | −0.37 | −1.42 | 5.64 |
| 1884 | ATLVT012XX776 | 45710 | NSESAASSNDNH | 0.28 | 2.27 | −0.23 |
| 1885 | ATLVT012XX2049 | 46983 | SSQAGASTNDNT | 0.41 | 0.4 | 1.94 |
| 1886 | ATLVT012XX2226 | 47160 | SSTSAGSSNDNT | −0.49 | 2.07 | 1.33 |
| 1887 | ATLVT012XX2303 | 47237 | TGAAAAASNDNH | −0.08 | 1.31 | 1.6 |
| 1888 | ATLVT012XX206 | 45140 | NGESAGASNDNH | −1.61 | −0.81 | 6.94 |
| 1889 | ATLVT012XX193 | 45127 | NGESAAASNDNA | 1.07 | −2.33 | 4.3 |
| 1890 | ATLVT012XX1157 | 46091 | SGAAAASSNDNH | −0.22 | 1.53 | 1.54 |
| 1891 | ATLVT012XX126 | 45060 | NGATGAATNDNT | 0.77 | 1.25 | 0.2 |
| 1892 | ATLVT012XX2767 | 47701 | TGTAGASTNDNA | 1.09 | 0.02 | 1.24 |
| 1893 | ATLVT012XX3361 | 48295 | TSTSAAATNDNA | −1.42 | 3.39 | 1.21 |
| 1894 | ATLVT012XX1635 | 46569 | SGTSAAATNDNT | 0.2 | 1.35 | 1.04 |
| 1895 | ATLVT012XX1418 | 46352 | SGETGAATNDNH | 0.25 | 1.01 | 1.39 |
| 1896 | ATLVT012XX2415 | 47349 | TGATAGAINDNT | −0.04 | −0.51 | 3.84 |
| 1897 | ATLVT012XX1596 | 46530 | SGTAAGASNDNT | 0.74 | 1.48 | −0.07 |
| 1898 | ATLVT012XX1168 | 46102 | SGAAAGSSNDNA | 0.77 | 0.98 | 0.53 |
| 1899 | ATLVT012XX2919 | 47853 | TSAAGGAINDNT | 0.13 | 1.1 | 1.45 |
| 1900 | ATLVT012XX2119 | 47053 | SSQTAASINDNA | 1.03 | −1.25 | 2.92 |
| 1901 | ATLVT012XX1269 | 46203 | SGATAGSINDNT | −1.04 | 3.1 | 0.88 |
| 1902 | ATLVT012XX2423 | 47357 | TGATGAASNDNH | 0.65 | 0.59 | 1.22 |
| 1903 | ATLVT012XX529 | 45463 | NGTTAAASNDNA | 2.13 | 0.05 | −0.65 |
| 1904 | ATLVT012XX2616 | 47550 | TGQAGAASNDNT | −1.54 | 3.74 | 0.9 |
| 1905 | ATLVT012XX2380 | 47314 | TGASGASSNDNA | 0.44 | 0.33 | 1.89 |
| 1906 | ATLVT012XX928 | 45862 | NSQSAGATNDNA | 1.54 | 0.35 | −0.04 |
| 1907 | ATLVT012XX3160 | 48094 | TSETGGSSNDNA | 1.94 | 0.77 | −1.26 |
| 1908 | ATLVT012XX2626 | 47560 | TGQAGGASNDNA | 0.07 | 0.62 | 2.13 |
| 1909 | ATLVT012XX3426 | 48360 | TSTTAGSSNDNT | −0.22 | 1.07 | 2.06 |
| 1910 | ATLVT012XX2716 | 47650 | IGQTGASSNDNA | 1.9 | −0.43 | 0.34 |
| 1911 | ATLVT012XX2851 | 47785 | TGTTAGSTNDNA | −0.14 | 1.01 | 1.98 |
| 1912 | ATLVT012XX47 | 44981 | NGAAGGSINDNH | 0.48 | 0.74 | 1.27 |
| 1913 | ATLVT012XX2583 | 47517 | TGETGGATNDNT | 1.11 | 0.18 | 0.9 |
| 1914 | ATLVT012XX2213 | 47147 | SSTSAASSNDNH | −0.51 | 1.57 | 1.89 |
| 1915 | ATLVT012XX685 | 45619 | NSATAGASNDNA | 1.52 | −0.61 | 1.21 |
| 1916 | ATLVT012XX2130 | 47064 | SSQTAGSSNDNT | 2.05 | −0.37 | −0.01 |
| 1917 | ATLVT012XX2889 | 47823 | TSAAAASTNDNT | −1.49 | 1.09 | 4.19 |
| 1918 | ATLVT012XX2363 | 47297 | TGASAGASNDNH | 0.64 | 0.51 | 1.27 |
| 1919 | ATLVT012XX2224 | 47158 | SSTSAGSSNDNA | 0.63 | 0.45 | 1.38 |
| 1920 | ATLVT012XX1826 | 46760 | SSATAAATNDNH | 2.37 | 0.18 | −1.27 |
| 1921 | ATLVT012XX1780 | 46714 | SSASAASSNDNA | 2.34 | −0.93 | 0.2 |
| 1922 | ATLVT012XX2039 | 46973 | SSQAGAASNDNH | 1.77 | −0.43 | 0.51 |
| 1923 | ATLVT012XX315 | 45249 | NGQAGAASNDNT | 0.9 | 0.45 | 0.88 |
| 1924 | ATLVT012XX3376 | 48310 | TSTSAGSSNDNA | −0.97 | 1.35 | 2.93 |
| 1925 | ATLVT012XX2793 | 47727 | TGTSAASTNQNT | 1.39 | 1.06 | −0.77 |
| 1926 | ATLVT012XX3034 | 47968 | TSEAAGASNDNA | −0.27 | 0.97 | 2.18 |
| 1927 | ATLVT012XX2592 | 47526 | TGQAAAASNDNT | −0.66 | −0.78 | 5.1 |
| 1928 | ATLVT012XX2407 | 47341 | TGATAASTNDNA | −0.69 | 2.66 | 0.73 |
| 1929 | ATLVT012XX1397 | 46331 | SGETAASSNDNH | 2.47 | −0.43 | −0.71 |
| 1930 | ATLVT012XX2676 | 47610 | TGQSGGASNDNT | 0.96 | 1.09 | −0.09 |
| 1931 | ATLVT012XX249 | 45183 | NGETAASSNDNT | 1.26 | 1.48 | −1.11 |
| 1932 | ATLVT012XX554 | 45488 | NGTTGAASNDNH | 1.16 | 0.43 | 0.43 |
| 1933 | ATLVT012XX2651 | 47585 | TGQSAGASNDNH | 0.46 | 0.46 | 1.57 |
| 1934 | ATLVT012XX2735 | 47669 | TGTAAAASNDNH | −1.13 | 3.26 | 0.69 |
| 1935 | ATLVT012XX1773 | 46707 | SSAAGGSINDNT | 0.97 | −0.05 | 1.34 |
| 1936 | ATLVT012XX971 | 45905 | NSQTAASINDNH | 1.25 | 0.65 | −0.05 |
| 1937 | ATLVT012XX1497 | 46431 | SGQSAASTNDNT | 1.09 | 1.41 | −0.74 |
| 1938 | ATLVT012XX488 | 45422 | NGTSAASSNDNH | 0.32 | 0.06 | 2.3 |
| 1939 | ATLVT012XX3090 | 48024 | TSESAGSSNDNT | −2.35 | 4.09 | 1.69 |
| 1940 | ATLVT012XX925 | 45859 | NSQSAGASNDNA | 0.36 | 1.36 | 0.55 |
| 1941 | ATLVT012XX2679 | 47613 | TGQSGGATNDNT | −0.36 | 1.38 | 1.75 |
| 1942 | ATLVT012XX1415 | 46349 | SGETGAASNDNH | 0.62 | 0.32 | 1.44 |
| 1943 | ATLVT012XX559 | 45493 | NGTTGASSNDNA | 0.32 | 0.77 | 1.35 |
| 1944 | ATLVT012XX441 | 45375 | NGTAAASSNDNT | −2.36 | 2.82 | 3.31 |
| 1945 | ATLVT012XX1553 | 46487 | SGQTAGSSNDNH | 0.68 | 1.81 | −0.61 |
| 1946 | ATLVT012XX3015 | 47949 | TSATGGATNDNT | 0.68 | 1.23 | 0.14 |
| 1947 | ATLVT012XX3292 | 48226 | TSQTGASSNDNA | −0.2 | 1.79 | 0.93 |
| 1948 | ATLVT012XX2813 | 47747 | TGTSGASSNDNH | 0.61 | 0.95 | 0.61 |
| 1949 | ATLVT012XX17 | 44951 | NGAAAGATNDNH | 0.04 | 0.79 | 1.79 |
| 1950 | ATLVT012XX3230 | 48164 | TSQSAGATNDNH | −0.17 | 1.87 | 0.76 |
| 1951 | ATLVT012XX2677 | 47611 | TGQSGGATNDNA | 1.66 | −0.25 | 0.33 |
| 1952 | ATLVT012XX408 | 45342 | NGQTAGSINDNT | 0.65 | 1.16 | 0.26 |
| 1953 | ATLVT012XX2984 | 47918 | TSATAASTNDNH | 0.87 | 1.24 | −0.23 |
| 1954 | ATLVT012XX847 | 45781 | NSETGASSNDNA | 2.41 | −0.15 | −1.08 |
| 1955 | ATLVT012XX3366 | 48300 | TSTSAASSNDNT | −2.19 | 1.46 | 4.73 |
| 1956 | ATLVT012XX1740 | 46674 | SSAAAGASNDNT | −0.32 | 1.64 | 1.28 |
| 1957 | ATLVT012XX2898 | 47832 | TSAAAGSSNDNT | −0.38 | 1.19 | 1.95 |
| 1958 | ATLVT012XX1900 | 46834 | SSEAGASSNDNA | 1.15 | 0.1 | 0.74 |
| 1959 | ATLVT012XX591 | 45525 | NSAAAGASNDNI | 0.35 | 0.82 | 1.16 |
| 1960 | ATLVT012XX655 | 45589 | NSASGASSNDNA | 1.4 | 0.21 | 0.14 |
| 1961 | ATLVT012XX2808 | 47742 | TGTSGAASNDNT | 0.48 | 1.01 | 0.69 |
| 1962 | ATLVT012XX2286 | 47220 | SSTTGASSNDNT | 0.62 | 1.58 | −0.3 |
| 1963 | ATLVT012XX2948 | 47882 | TSASAGSTNDNH | 0.43 | 1.39 | 0.26 |
| 1964 | ATLVT012XX1216 | 46150 | SGASAGSSNDNA | 1.12 | −0.22 | 1.15 |
| 1965 | ATLVT012XX3289 | 48223 | TSQTGAATNDNA | 0.37 | 0.83 | 1.08 |
| 1966 | ATLVT012XX2714 | 47648 | TGQTGAATNDNH | 0.17 | 0.41 | 1.95 |
| 1967 | ATLVT012XX1403 | 46337 | SGETAGASNDNH | −0.02 | 1.68 | 0.63 |
| 1968 | ATLVT012XX2416 | 47350 | TGATAGSSNDNA | 1.85 | −0.5 | 0.24 |
| 1969 | ATLVT012XX734 | 45668 | NSEAAGASNDNH | 0.19 | 1.1 | 1.01 |
| 1970 | ATLVT012XX401 | 45335 | NGQTAGAINDNH | 1.28 | 1.25 | −1.05 |
| 1971 | ATLVT012XX2490 | 47424 | TGEAGGSSNDNT | 0.31 | 1.69 | 0.03 |
| 1972 | ATLVT012XX337 | 45271 | NGQSAAASNDNA | 0.22 | 0.6 | 1.59 |
| 1973 | ATLVT012XX2754 | 47688 | TGTAAGSSNDNT | 0.78 | 0.89 | 0.26 |
| 1974 | ATLVT012XX588 | 45522 | NSAAAASINDNT | −1.9 | −0.48 | 6.6 |
| 1975 | ATLVT012XX3370 | 48304 | TSTSAGASNDNA | −0.67 | 1.84 | 1.5 |
| 1976 | ATLVT012XX2524 | 47458 | TGESGASSNDNA | −1.02 | −0.97 | 5.71 |
| 1977 | ATLVT012XX3368 | 48302 | TSTSAASTNDNH | −0.83 | 1.55 | 2.14 |
| 1978 | ATLVT012XX2486 | 47420 | TGEAGGATNDNH | 0.44 | 1.05 | 0.61 |
| 1979 | ATLVT012XX2215 | 47149 | SSTSAASINDNA | 0.45 | 0.86 | 0.83 |
| 1980 | ATLVT012XX2617 | 47551 | TGQAGAATNDNA | −0.29 | 1.19 | 1.68 |
| 1981 | ATLVT012XX2859 | 47793 | TGTTGAATNDNT | 0.59 | 0.23 | 1.39 |
| 1982 | ATLVT012XX291 | 45225 | NGQAAAASNDNT | 1.12 | −1.95 | 3.28 |
| 1983 | ATLVT012XX3023 | 47957 | TSEAAAASNDNH | 2.03 | −0.49 | −0.13 |
| 1984 | ATLVT012XX1546 | 46480 | SGQTAGASNDNA | 0.04 | 1.59 | 0.58 |
| 1985 | ATLVT012XX1741 | 46675 | SSAAAGATNDNA | −0.25 | 1.64 | 1.02 |
| 1986 | ATLVT012XX1849 | 46783 | SSATGAATNDNA | 0.92 | 0.79 | 0.09 |
| 1987 | ATLVT012XX1825 | 46759 | SSATAAATNDNA | 2.64 | −0.28 | −1.48 |
| 1988 | ATLVT012XX931 | 45865 | NSQSAGSSNDNA | 2.21 | 0.14 | −1.29 |
| 1989 | ATLVT012XX3268 | 48202 | TSQTAASSNDNA | 0.12 | −1.4 | 4.27 |
| 1990 | ATLVT012XX2556 | 47490 | TGETAGASNDNT | −1.26 | 3.94 | −0.24 |
| 1991 | ATLVT012XX2786 | 47720 | TGTSAAATNDNH | 0.7 | 0.47 | 0.87 |
| 1992 | ATLVT012XX1592 | 46526 | SGTAAASTNDNH | −0.35 | 0.13 | 3.1 |
| 1993 | ATLVT012XX700 | 45634 | NSATGAATNDNA | 0.12 | 0.07 | 2.37 |
| 1994 | ATLVT012XX1212 | 46146 | SGASAGASNDNT | 0.83 | 0.23 | 0.94 |
| 1995 | ATLVT012XX1847 | 46781 | SSATGAASNDNH | 0.45 | 1.77 | −0.38 |
| 1996 | ATLVT012XX2371 | 47305 | TGASAGSINDNA | −2.06 | 0.67 | 5.33 |
| 1997 | ATLVT012XX1259 | 46193 | SGATAGASNDNH | 1.09 | 0.52 | 0.12 |
| 1998 | ATLVT012XX648 | 45582 | NSASAGSTNDNT | 1.31 | 0 | 0.39 |
| 1999 | ATLVT012XX1797 | 46731 | SSASAGSINDNT | −0.34 | 3.19 | −0.88 |
| 2000 | ATLVT012XX3425 | 48359 | TSTTAGSSNDNH | 0.82 | 0.49 | 0.61 |
| 2001 | ATLVT012XX1267 | 46201 | SGATAGSINDNA | 0.18 | 2.46 | −0.87 |
| 2002 | ATLVT012XX2181 | 47115 | SSTAAGSINDNT | −0.8 | 1.69 | 1.8 |
| 2003 | ATLVT012XX202 | 45136 | NGESAASTNDNA | 0.24 | −0.43 | 2.72 |
| 2004 | ATLVT012XX55 | 44989 | NGASAASSNDNA | 1.69 | −0.23 | −0.03 |
| 2005 | ATLVT012XX104 | 45038 | NGATAASSNDNH | 0.8 | 1.24 | −0.39 |
| 2006 | ATLVT012XX426 | 45360 | NGQTGGAINDNT | −0.92 | 0.92 | 2.95 |
| 2007 | ATLVT012XX1233 | 46167 | SGASGASTNDNT | 1.58 | −0.73 | 0.79 |
| 2008 | ATLVT012XX438 | 45372 | NGTAAAATNDNT | −1.54 | −0.39 | 5.7 |
| 2009 | ATLVT012XX2373 | 47307 | TGASAGSINDNT | −0.67 | 0.37 | 3.22 |
| 2010 | ATLVT012XX1200 | 46134 | SGASAAASNDNT | 1.67 | −0.93 | 0.88 |
| 2011 | ATLVT012XX34 | 44968 | NGAAGASTNDNA | 0.13 | 0.87 | 1.2 |
| 2012 | ATLVT012XX3144 | 48078 | TSETGAASNDNT | 1.2 | −0.11 | 0.63 |
| 2013 | ATLVT012XX2045 | 46979 | SSQAGASSNDNH | 1.9 | −0.36 | −0.25 |
| 2014 | ATLVT012XX1276 | 46210 | SGATGASSNDNA | 1.25 | 0.66 | −0.47 |
| 2015 | ATLVT012XX327 | 45261 | NGQAGGASNDNT | 0.71 | 0.18 | 1.08 |
| 2016 | ATLVT012XX2896 | 47830 | TSAAAGSSNDNA | 0.15 | 1.52 | 0.31 |
| 2017 | ATLVT012XX3096 | 48030 | TSESGAASNDNI | −0.22 | 0.52 | 2.23 |
| 2018 | ATLVT012XX1213 | 46147 | SGASAGATNDNA | 0.82 | −0.05 | 1.17 |
| 2019 | ATLVT012XX189 | 45123 | NGEAGGSSNDNT | 2.09 | −0.46 | −0.47 |
| 2020 | ATLVT012XX229 | 45163 | NGESGGASNDNA | 0.86 | 1.53 | −0.93 |
| 2021 | ATLVT012XX128 | 45062 | NGATGASSNDNH | 0.6 | 0.54 | 0.76 |
| 2022 | ATLVT012XX30 | 44964 | NGAAGAATNDNT | −0.07 | 0.89 | 1.47 |
| 2023 | ATLVT012XX3363 | 48297 | TSTSAAATNDNT | −1.88 | 3.18 | 1.59 |
| 2024 | ATLVT012XX540 | 45474 | NGTTAASINDNT | 0.33 | 0.2 | 1.62 |
| 2025 | ATLVT012XX843 | 45777 | NSETGAASNDNT | 0.86 | −0.22 | 1.25 |
| 2026 | ATLVT012XX37 | 44971 | NGAAGGASNDNA | 0.06 | 0.69 | 1.45 |
| 2027 | ATLVT012XX894 | 45828 | NSQAGAATNDNT | 0.82 | 0.59 | 0.29 |
| 2028 | ATLVT012XX1541 | 46475 | SGQTAASSNDNH | 0.49 | 0.43 | 1.06 |
| 2029 | ATLVT012XX943 | 45877 | NSQSGASSNDNA | 0.88 | 0.98 | −0.34 |
| 2030 | ATLVT012XX209 | 45143 | NGESAGAINDNH | −1.05 | 1.04 | 2.87 |
| 2031 | ATLVT012XX2888 | 47822 | TSAAAASTNDNH | −1.93 | 3.85 | 0.77 |
| 2032 | ATLVT012XX2581 | 47515 | TGETGGATNDNA | 0.9 | 0.94 | −0.34 |
| 2033 | ATLVT012XX927 | 45861 | NSQSAGASNDNT | 1.02 | 0.39 | 0.15 |
| 2034 | ATLVT012XX191 | 45125 | NGEAGGSINDNH | 0.53 | 2.24 | −1.4 |
| 2035 | ATLVT012XX2790 | 47724 | TGTSAASSNDNT | 0.58 | 0.41 | 0.87 |
| 2036 | ATLVT012XX1280 | 46214 | SGATGASTNDNH | 2.13 | −0.17 | −1.05 |
| 2037 | ATLVT012XX2402 | 47336 | TGATAAATNDNH | 1.25 | 0.26 | −0.1 |
| 2038 | ATLVT012XX549 | 45483 | NGTTAGSSNDNT | −0.89 | 1.53 | 1.93 |
| 2039 | ATLVT012XX836 | 45770 | NSETAGSSNDNH | 0.64 | 0.68 | 0.39 |
| 2040 | ATLVT012XX3035 | 47969 | TSEAAGASNDNH | −0.58 | 0.92 | 2.18 |
| 2041 | ATLVT012XX1393 | 46327 | SGETAAATNDNA | 1.87 | −0.16 | −0.65 |
| 2042 | ATLVT012XX3021 | 47955 | TSATGGSINDNT | 0.92 | 0.87 | −0.34 |
| 2043 | ATLVT012XX2552 | 47486 | TGETAASTNDNH | 1.19 | −0.28 | 0.68 |
| 2044 | ATLVT012XX1367 | 46301 | SGESGAASNDNH | 0.39 | 1.34 | −0.04 |
| 2045 | ATLVT012XX1174 | 46108 | SGAAGAASNDNA | −0.08 | 0.66 | 1.64 |
| 2046 | ATLVT012XX392 | 45326 | NGQTAASSNDNH | 0.54 | −0.4 | 1.93 |
| 2047 | ATLVT012XX2861 | 47795 | TGTTGASSNDNH | 0.73 | 0.61 | 0.3 |
| 2048 | ATLVT012XX2947 | 47881 | TSASAGSTNDNA | −0.29 | 1.94 | 0.35 |
| 2049 | ATLVT012XX1228 | 46162 | SGASGASSNDNA | 1.86 | −0.46 | −0.25 |
| 2050 | ATLVT012XX3108 | 48042 | TSESGGASNDNT | 0.16 | 1.07 | 0.68 |
| 2051 | ATLVT012XX3231 | 48165 | TSQSAGATNDNT | −0.11 | 0.93 | 1.33 |
| 2052 | ATLVT012XX1591 | 46525 | SGTAAASTNDNA | −0.6 | 0.39 | 2.84 |
| 2053 | ATLVT012XX725 | 45659 | NSEAAAATNDNH | 1.97 | 0.26 | −1.4 |
| 2054 | ATLVT012XX2165 | 47099 | SSTAAASSNDNH | −1.13 | 3.16 | 0.19 |
| 2055 | ATLVT012XX962 | 45896 | NSQTAAASNDNH | −0.26 | −1.23 | 4.36 |
| 2056 | ATLVT012XX1881 | 46815 | SSEAAASTNQNT | 1.01 | 0.42 | 0.04 |
| 2057 | ATLVT012XX562 | 45496 | NGTTGASTNDNA | −0.66 | 2.14 | 0.69 |
| 2058 | ATLVT012XX3249 | 48183 | TSQSGASTNDNT | −0.61 | 0.2 | 3.09 |
| 2059 | ATLVT012XX2748 | 47682 | TGTAAGASNDNT | 0 | 1.19 | 0.77 |
| 2060 | ATLVT012XX1420 | 46354 | SGETGASSNDNA | 0.09 | 1.5 | 0.21 |
| 2061 | ATLVT01XXX1800 | 46734 | SSASGAASNDNT | 0.27 | 0.82 | 0.78 |
| 2062 | ATLVT012XX2125 | 47059 | SSQTAGATNDNA | 0.73 | −0.05 | 1.1 |
| 2063 | ATLVT012XX1901 | 46835 | SSEAGASSNDNH | 0.9 | −0.29 | 1.13 |
| 2064 | ATLVT012XX117 | 45051 | NGATAGSSNDNT | 1.01 | −0.15 | 0.74 |
| 2065 | ATLVT012XX1161 | 46095 | SGAAAASINDNT | −0.69 | 1.36 | 1.71 |
| 2066 | ATLVT012XX1883 | 46817 | SSEAAGASNDNH | 0 | 1.3 | 0.6 |
| 2067 | ATLVT012XX2361 | 47295 | TGASAASTNDNT | −0.06 | 2.36 | −0.69 |
| 2068 | ATLVT012XX3018 | 47952 | TSATGGSSNDNT | 1.22 | 0.35 | −0.29 |
| 2069 | ATLVT012XX2131 | 47065 | SSQTAGSTNDNA | 0.37 | 0.63 | 0.79 |
| 2070 | ATLVT012XX1630 | 46564 | SGTSAAASNDNA | 0.56 | −0.05 | 1.34 |
| 2071 | ATLVT012XX309 | 45243 | NGQAAGSSNDNT | 0.21 | 0.47 | 1.26 |
| 2072 | ATLVT012XX2855 | 47789 | TGTTGAASNDNH | 0.47 | 0.7 | 0.52 |
| 2073 | ATLVT012XX1338 | 46272 | SGEAGGSSNDNT | 0.9 | −0.39 | 1.19 |
| 2074 | ATLVT012XX214 | 45148 | NGESAGSTNDNA | −0.65 | 1.76 | 1.07 |
| 2075 | ATLVT012XX2040 | 46974 | SSQAGAASNDNT | 0.65 | 0.59 | 0.36 |
| 2076 | ATLVT012XX2830 | 47764 | TGTTAAASNDNA | 2.36 | −0.2 | −1.57 |
| 2077 | ATLVT012XX759 | 45693 | NSEAGGASNDNT | −0.15 | 0.62 | 1.66 |
| 2078 | ATLVT012XX1014 | 45948 | NSTAAAATNDNT | 0.3 | −0.69 | 2.58 |
| 2079 | ATLVT012XX3154 | 48088 | TSETGGASNDNA | 0.73 | 0.56 | 0.24 |
| 2080 | ATLVT012XX1973 | 46907 | SSETAASSNDNH | 0.14 | −0.03 | 2.01 |
| 2081 | ATLVT012XX3291 | 48225 | TSQTGAATNDNT | 0.17 | −0.42 | 2.46 |
| 2082 | ATLVT012XX1835 | 46769 | SSATAGASNDNH | −0.14 | 2.05 | −0.2 |
| 2083 | ATLVT012XX301 | 45235 | NGQAAGASNDNA | −0.3 | 0.91 | 1.54 |
| 2084 | ATLVT012XX32 | 44966 | NGAAGASSNDNH | −0.3 | 1.09 | 1.31 |
| 2085 | ATLVT012XX2384 | 47318 | IGASGASTNDNH | −0.34 | 1.7 | 0.59 |
| 2086 | ATLVT012XX841 | 45775 | NSETGAASNDNA | 1.3 | −0.43 | 0.51 |
| 2087 | ATLVT012XX361 | 45295 | NGQSGAASNDNA | 1.18 | −0.45 | 0.74 |
| 2088 | ATLVT012XX2111 | 47045 | SSQTAAASNDNH | −0.78 | 0.15 | 3.33 |
| 2089 | ATLVT012XX2267 | 47201 | SSTTAGASNDNH | 0.17 | 0.76 | 0.91 |
| 2090 | ATLVT012XX2304 | 47238 | TGAAAAASNDNT | −0.18 | 0.85 | 1.4 |
| 2091 | ATLVT012XX1788 | 46722 | SSASAGASNDNT | 0.55 | 0.95 | 0.01 |
| 2092 | ATLVT012XX2127 | 47061 | SSQTAGATNDNT | 1.77 | −0.81 | 0.17 |
| 2093 | ATLVT012XX987 | 45921 | NSQTGAASNDNT | 1.17 | 0.27 | −0.19 |
| 2094 | ATLVT012XX3119 | 48053 | TSETAAASNDNH | −0.4 | 1.01 | 1.55 |
| 2095 | ATLVT012XX1225 | 46159 | SGASGAATNDNA | 0.22 | 0.22 | 1.49 |
| 2096 | ATLVT012XX1991 | 46925 | SSETGAASNDNH | 0.75 | −0.01 | 0.86 |
| 2097 | ATLVT012XX2707 | 47641 | TGQTAGSTNDNA | 0.43 | 1.1 | −0.01 |
| 2098 | ATLVT012XX2569 | 47503 | TGETGAATNDNA | −1.16 | 1.96 | 1.61 |
| 2099 | ATLVT012XX3279 | 48213 | TSQTAGAINDNT | 0.53 | 0.1 | 1.1 |
| 2100 | ATLVT012XX2812 | 47746 | TGTSGASSNDNA | 1.91 | 0.25 | −1.47 |
| 2101 | ATLVT012XX2314 | 47248 | TGAAAGASNDNA | −0.03 | 0.74 | 1.22 |
| 2102 | ATLVT012XX456 | 45390 | NGTAAGSTNDNT | 0.64 | 0.15 | 0.82 |
| 2103 | ATLVT012XX2032 | 46966 | SSQAAGSSNDNA | 0.3 | 0.64 | 0.77 |
| 2104 | ATLVT012XX2368 | 47302 | TGASAGSSNDNA | −0.62 | 0.92 | 2 |
| 2105 | ATLVT012XX3095 | 48029 | TSESGAASNDNH | −0.2 | 0.44 | 1.89 |
| 2106 | ATLVT012XX996 | 45930 | NSQTGASINDNT | 0.84 | 0.87 | −0.46 |
| 2107 | ATLVT012XX2657 | 47591 | TGQSAGSSNDNH | 0.36 | 0.89 | 0.34 |
| 2108 | ATLVT012XX2998 | 47932 | TSATGAASNDNA | 0.89 | −0.08 | 0.66 |
| 2109 | ATLVT012XX1698 | 46632 | SGTTAGSSNDNT | −1.25 | 1.92 | 1.75 |
| 2110 | ATLVT012XX544 | 45478 | NGTTAGAINDNA | 0.3 | −0.3 | 1.94 |
| 2111 | ATLVT012XX995 | 45929 | NSQTGASTNDNH | 0.74 | 1.11 | −0.63 |
| 2112 | ATLVT012XX1281 | 46215 | SGATGASTNDNT | 2.56 | −0.72 | −1.39 |
| 2113 | ATLVT012XX2325 | 47259 | TGAAAGSTNDNT | −0.17 | 0.69 | 1.46 |
| 2114 | ATLVT012XX2848 | 47782 | TGTTAGSSNDNA | 0 | 0.92 | 0.87 |
| 2115 | ATLVT012XX2273 | 47207 | SSTTAGSSNDNH | 1.3 | 0.72 | −1.09 |
| 2116 | ATLVT012XX300 | 45234 | NGQAAASINDNT | 1.51 | −0.42 | 0 |
| 2117 | ATLVT012XX65 | 44999 | NGASAGATNDNH | 0.07 | 2.21 | −0.91 |
| 2118 | ATLVT012XX1970 | 46904 | SSETAAATNDNH | 0.38 | −0.16 | 1.59 |
| 2119 | ATLVT012XX646 | 45580 | NSASAGSINDNA | 0.49 | 0.63 | 0.38 |
| 2120 | ATLVT012XX963 | 45897 | NSQTAAASNDNT | 1.82 | −1.87 | 1.32 |
| 2121 | ATLVT012XX405 | 45339 | NGQTAGSSNDNT | −0.25 | 1.48 | 0.56 |
| 2122 | ATLVT012XX2567 | 47501 | TGETGAASNDNH | 0.27 | 0.26 | 1.23 |
| 2123 | ATLVT012XX3369 | 48303 | TSTSAASTNDNT | −0.42 | 0.36 | 2.26 |
| 2124 | ATLVT012XX2713 | 47647 | TGQTGAATNDNA | −0.61 | 0.49 | 2.43 |
| 2125 | ATLVT012XX2985 | 47919 | TSATAASTNDNT | 0.61 | 0.5 | 0.32 |
| 2126 | ATLVT012XX1795 | 46729 | SSASAGSTNDNA | −0.09 | 1.66 | 0.03 |
| 2127 | ATLVT012XX1979 | 46913 | SSETAGASNDNH | 0.6 | 0.23 | 0.68 |
| 2128 | ATLVT012XX2143 | 47077 | SSQTGASINDNA | −0.35 | 0.29 | 2.24 |
| 2129 | ATLVT012XX1548 | 46482 | SGQTAGASNDNT | 0.52 | 0.73 | 0.16 |
| 2130 | ATLVT012XX653 | 45587 | NSASGAATNDNH | 0.6 | 0.66 | 0.12 |
| 2131 | ATLVT012XX2787 | 47721 | TGTSAAATNDNT | −0.28 | 1.32 | 0.78 |
| 2132 | ATLVT012XX371 | 45305 | NGQSGASTNDNH | −0.5 | 1.12 | 1.41 |
| 2133 | ATLVT012XX1743 | 46677 | SSAAAGAINDNT | −0.14 | 1.23 | 0.64 |
| 2134 | ATLVT012XX1833 | 46767 | SSATAASINDNT | 1.96 | −0.75 | −0.41 |
| 2135 | ATLVT012XX3066 | 48000 | TSEAGGSSNDNT | 0.28 | 0.11 | 1.37 |
| 2136 | ATLVT012XX444 | 45378 | NGTAAASTNDNT | 0.77 | 0.15 | 0.48 |
| 2137 | ATLVT012XX920 | 45854 | NSQSAASSNDNH | 0.05 | 1.11 | 0.47 |
| 2138 | ATLVT012XX2047 | 46981 | SSQAGASINDNA | 1.07 | −0.49 | 0.78 |
| 2139 | ATLVT012XX2700 | 47634 | TGQTAGASNDNT | 0.25 | 0.54 | 0.85 |
| 2140 | ATLVT012XX873 | 45807 | NSQAAASSNDNT | 0.87 | 0.59 | −0.29 |
| 2141 | ATLVT012XX1940 | 46874 | SSESAGSTNDNH | −0.4 | 1.91 | 0.2 |
| 2142 | ATLVT012XX1654 | 46588 | SGTSGAASNDNA | 0.37 | 0.26 | 1 |
| 2143 | ATLVT012XX2308 | 47242 | TGAAAASSNDNA | −0.71 | 1.43 | 1.34 |
| 2144 | ATLVT012XX3184 | 48118 | TSQAAGSSNDNA | 0.17 | 0.41 | 1.14 |
| 2145 | ATLVT012XX127 | 45061 | NGATGASSNDNA | 1.37 | −0.13 | −0.22 |
| 2146 | ATLVT012XX2900 | 47834 | TSAAAGSINDNH | −0.1 | 1.2 | 0.57 |
| 2147 | ATLVT012XX3417 | 48351 | ISTTAASINDNT | −0.76 | 0.23 | 2.96 |
| 2148 | ATLVT012XX2706 | 47640 | TGQTAGSSNDNT | −1.3 | 3.26 | −0.02 |
| 2149 | ATLVT012XX3145 | 48079 | TSETGAATNDNA | −0.3 | 0.97 | 1.19 |
| 2150 | ATLVT012XX3320 | 48254 | TSTAAASTNDNH | −1.11 | 2.05 | 1.18 |
| 2151 | ATLVT012XX2076 | 47010 | SSQSAGASNDNT | 1.22 | −0.36 | 0.3 |
| 2152 | ATLVT012XX2944 | 47878 | TSASAGSSNDNA | 0.54 | 0.4 | 0.48 |
| 2153 | ATLVT012XX2072 | 47006 | SSQSAASINDNH | 0.72 | 0.29 | 0.31 |
| 2154 | ATLVT012XX2488 | 47422 | TGEAGGSSNDNA | 1.34 | 0.02 | −0.42 |
| 2155 | ATLVT012XX775 | 45709 | NSESAASSNDNA | 2.03 | −0.59 | −0.84 |
| 2156 | ATLVT012XX2080 | 47014 | SSQSAGSSNDNA | 0.55 | 1.11 | −0.49 |
| 2157 | ATLVT012XX2845 | 47779 | TGTTAGAINDNA | 0.5 | 0.52 | 0.35 |
| 2158 | ATLVT012XX150 | 45084 | NGEAAAATNDNT | 3.01 | −1.58 | −1.25 |
| 2159 | ATLVT012XX2769 | 47703 | TGTAGASTNDNT | 0.69 | −0.05 | 0.75 |
| 2160 | ATLVT012XX2457 | 47391 | TGEAAASTNDNT | 0.6 | 0.99 | −0.44 |
| 2161 | ATLVT012XX394 | 45328 | NGQTAASTNDNA | −1.23 | −0.01 | 3.98 |
| 2162 | ATLVT012XX1492 | 46426 | SGQSAASSNDNA | 0.75 | 0.64 | −0.26 |
| 2163 | ATLVT012XX3041 | 47975 | TSEAAGSSNDNH | 0.09 | 0.65 | 0.86 |
| 2164 | ATLVT012XX589 | 45523 | NSAAAGASNDNA | −0.06 | 0.84 | 0.88 |
| 2165 | ATLVT012XX1589 | 46523 | SGTAAASSNDNH | −1.04 | −0.36 | 4.09 |
| 2166 | ATLVT012XX3362 | 48296 | TSTSAAATNDNH | −1.18 | 3.13 | −0.16 |
| 2167 | ATLVT012XX590 | 45524 | NSAAAGASNDNH | −0.51 | 1.6 | 0.65 |
| 2168 | ATLVT012XX824 | 45758 | NSETAASSNDNH | −1.87 | 4.24 | −0.41 |
| 2169 | ATLVT012XX2038 | 46972 | SSQAGAASNDNA | 0.17 | 0.14 | 1.37 |
| 2170 | ATLVT012XX2650 | 47584 | TGQSAGASNDNA | 0.88 | −0.43 | 0.87 |
| 2171 | ATLVT012XX2134 | 47068 | SSQTGAASNDNA | −0.08 | −0.18 | 2.2 |
| 2172 | ATLVT012XX2120 | 47054 | SSQTAASINDNH | −0.07 | −0.03 | 2 |
| 2173 | ATLVT012XX1634 | 46568 | SGTSAAATNDNH | 0.76 | 0.43 | −0.03 |
| 2174 | ATLVT012XX81 | 45015 | NGASGASSNDNT | −1.14 | 1.52 | 1.83 |
| 2175 | ATLVT012XX2746 | 47680 | TGTAAGASNDNA | −0.78 | 2.38 | 0.11 |
| 2176 | ATLVT012XX1987 | 46921 | SSETAGSINDNA | 1.46 | −1.3 | 0.98 |
| 2177 | ATLVT012XX1489 | 46423 | SGQSAAATNDNA | 0.37 | 1.2 | −0.36 |
| 2178 | ATLVT012XX914 | 45848 | NSQSAAASNDNH | 0.89 | −0.52 | 0.95 |
| 2179 | ATLVT012XX1759 | 46693 | SSAAGASINDNA | 0.1 | 0.17 | 1.41 |
| 2180 | ATLVT012XX680 | 45614 | NSATAASSNDNH | 1.47 | −0.85 | 0.37 |
| 2181 | ATLVT012XX3101 | 48035 | TSESGASSNDNH | −0.51 | 0.66 | 1.83 |
| 2182 | ATLVT012XX2450 | 47384 | TGEAAAATNDNH | 0.98 | 0.74 | −0.84 |
| 2183 | ATLVT012XX1363 | 46297 | SGESAGSINDNA | −0.14 | 1.32 | 0.33 |
| 2184 | ATLVT012XX3152 | 48086 | TSETGASTNDNH | 1.18 | 0.19 | −0.48 |
| 2185 | ATLVT012XX322 | 45256 | NGQAGASTNDNA | 1.04 | −0.35 | 0.45 |
| 2186 | ATLVT012XX2992 | 47926 | TSATAGSSNDNA | 1.15 | −0.29 | 0.17 |
| 2187 | ATLVT012XX3092 | 48026 | TSESAGSTNDNH | −1.82 | 1.26 | 3.29 |
| 2188 | ATLVT012XX930 | 45864 | NSQSAGAINDNT | 0.62 | 0.37 | 0.25 |
| 2189 | ATLVT012XX1450 | 46384 | SGQAAGASNDNA | −1.05 | 1.98 | 1.04 |
| 2190 | ATLVT012XX2483 | 47417 | TGEAGGASNDNH | 1.01 | 0.09 | −0.07 |
| 2191 | ATLVT012XX2857 | 47791 | TGTTGAATNDNA | −0.45 | 0.51 | 1.88 |
| 2192 | ATLVT012XX2168 | 47102 | SSTAAASTNDNH | −0.82 | 2.45 | 0.01 |
| 2193 | ATLVT012XX1841 | 46775 | SSATAGSSNDNH | 2.31 | −0.71 | −1.29 |
| 2194 | ATLVT012XX609 | 45543 | NSAAGASSNDNT | −0.66 | 1.95 | 0.36 |
| 2195 | ATLVT012XX3365 | 48299 | TSTSAASSNDNH | −1.82 | 1.55 | 2.86 |
| 2196 | ATLVT012XX3432 | 48366 | TSTTGAASNDNT | 0.62 | 0.16 | 0.46 |
| 2197 | ATLVT012XX966 | 45900 | NSQTAAATNDNT | 1.75 | −1.8 | 1.04 |
| 2198 | ATLVT012XX1944 | 46878 | SSESGAASNDNT | 0.35 | 0.2 | 0.88 |
| 2199 | ATLVT012XX2507 | 47441 | TGESAGASNDNH | −1.8 | −0.25 | 5.14 |
| 2200 | ATLVT012XX2519 | 47453 | TGESGAASNDNH | 0.4 | −0.04 | 1.1 |
| 2201 | ATLVT012XX2424 | 47358 | TGATGAASNDNT | 0.18 | 0.54 | 0.73 |
| 2202 | ATLVT012XX222 | 45156 | NGESGAATNDNT | 0.76 | −1.23 | 2 |
| 2203 | ATLVT012XX935 | 45869 | NSQSAGSTNDNH | 1.83 | 0.02 | −1.45 |
| 2204 | ATLVT012XX110 | 45044 | NGATAGASNDNH | 0.43 | 0.3 | 0.57 |
| 2205 | ATLVT012XX2537 | 47471 | TGESGGSSNDNH | −0.44 | −0.15 | 2.64 |
| 2206 | ATLVT012XX758 | 45692 | NSEAGGASNDNH | −0.58 | 1.22 | 1.12 |
| 2207 | ATLVT012XX1538 | 46472 | SGQTAAATNDNH | 0.45 | 1.84 | −1.46 |
| 2208 | ATLVT012XX1209 | 46143 | SGASAASTNDNT | 1.58 | −1.02 | 0.29 |
| 2209 | ATLVT012XX447 | 45381 | NGTAAGASNDNT | 0.1 | 0.71 | 0.6 |
| 2210 | ATLVT012XX2266 | 47200 | SSTTAGASNDNA | 1.16 | 2.44 | 0.53 |
| 2211 | ATLVT012XX1918 | 46852 | SSESAAASNDNA | 1.3 | −0.24 | −0.25 |
| 2212 | ATLVT012XX3146 | 48080 | TSETGAATNDNH | 0.08 | 0.75 | 0.59 |
| 2213 | ATLVT012XX3274 | 48208 | TSQTAGASNDNA | 0.09 | 0.59 | 0.78 |
| 2214 | ATLVT012XX3410 | 48344 | TSTTAAATNDNH | −0.68 | 2.64 | −0.56 |
| 2215 | ATLVT012XX3100 | 48034 | TSESGASSNDNA | −1.77 | −0.71 | 5.62 |
| 2216 | ATLVT012XX1617 | 46551 | SGTAGASTNDNT | 0.53 | −0.45 | 1.34 |
| 2217 | ATLVT012XX2937 | 47871 | TSASAASTNDNT | −0.31 | 1.01 | 0.9 |
| 2218 | ATLVT012XX3269 | 48203 | TSQTAASSNDNH | −0.33 | −0.05 | 2.29 |
| 2219 | ATLVT012XX1306 | 46240 | SGEAAGASNDNA | −0.33 | 1.04 | 0.89 |
| 2220 | ATLVT012XX760 | 45694 | NSEAGGATNDNA | −0.96 | 2.18 | 0.5 |
| 2221 | ATLVT012XX1748 | 46682 | SSAAAGSINDNH | 0.12 | 0.59 | 0.7 |
| 2222 | ATLVT012XX702 | 45636 | NSATGAATNDNT | 1.05 | −0.18 | 0.07 |
| 2223 | ATLVT012XX82 | 45016 | NGASGASTNDNA | 0.13 | 0.44 | 0.84 |
| 2224 | ATLVT012XX3086 | 48020 | TSESAGATNDNH | −1.2 | 1.69 | 1.51 |
| 2225 | ATLVT012XX2074 | 47008 | SSQSAGASNDNA | −0.11 | 0.88 | 0.68 |
| 2226 | ATLVT012XX592 | 45526 | NSAAAGATNDNA | −0.61 | 1.25 | 1.06 |
| 2227 | ATLVT012XX2604 | 47538 | TGQAAGASNDNT | −0.66 | 0.63 | 1.93 |
| 2228 | ATLVT012XX2610 | 47544 | TGQAAGSSNDNT | −0.62 | 0.74 | 1.72 |
| 2229 | ATLVT012XX838 | 45772 | NSETAGSINDNA | 1.8 | −0.77 | −0.5 |
| 2230 | ATLVT012XX1417 | 46351 | SGETGAATNDNA | −1.78 | −1.01 | 5.94 |
| 2231 | ATLVT012XX988 | 45922 | NSQTGAATNDNA | −1.01 | 2.1 | 0.61 |
| 2232 | ATLVT012XX2261 | 47195 | SSTTAASSNDNH | 0.13 | 0.62 | 0.56 |
| 2233 | ATLVT012XX936 | 45870 | NSQSAGSINDNT | 1.27 | 0.19 | −0.84 |
| 2234 | ATLVT012XX1971 | 46905 | SSETAAATNDNT | −1.47 | −0.84 | 5.18 |
| 2235 | ATLVT012XX2784 | 47718 | TGTSAAASNDNT | −0.02 | −0.2 | 1.87 |
| 2236 | ATLVT012XX631 | 45565 | NSASAASSNDNA | −0.75 | 1.3 | 1.2 |
| 2237 | ATLVT012XX1368 | 46302 | SGESGAASNDNT | −0.12 | 0.58 | 1.04 |
| 2238 | ATLVT012XX2987 | 47921 | TSATAGASNDNH | −0.77 | 1.12 | 1.46 |
| 2239 | ATLVT012XX1830 | 46764 | SSATAASSNDNT | −0.7 | −0.51 | 3.44 |
| 2240 | ATLVT012XX1761 | 46695 | SSAAGASTNDNI | −0.01 | 0.26 | 1.27 |
| 2241 | ATLVT01XXX1928 | 46862 | SSESAASTNDNH | 0.28 | 0.77 | 0.12 |
| 2242 | ATLVT012XX2427 | 47361 | TGATGAATNDNT | −0.33 | 0.39 | 1.64 |
| 2243 | ATLVT012XX2365 | 47299 | TGASAGATNDNA | 0.41 | 0.07 | 0.79 |
| 2244 | ATLVT012XX1261 | 46195 | SGATAGAINDNA | 2.08 | −1.25 | −0.4 |
| 2245 | ATLVT012XX2978 | 47912 | TSATAAATNDNH | 0.56 | 0.94 | −0.6 |
| 2246 | ATLVT012XX1309 | 46243 | SGEAAGATNDNA | −0.98 | −0.58 | 3.99 |
| 2247 | ATLVT012XX2697 | 47631 | TGQTAASTNDNT | 0.49 | −0.28 | 1.07 |
| 2248 | ATLVT012XX466 | 45400 | NGTAGASTNDNA | 0.12 | 0.39 | 0.83 |
| 2249 | ATLVT012XX185 | 45119 | NGEAGGATNDNH | 0.45 | 0.83 | −0.29 |
| 2250 | ATLVT012XX681 | 45615 | NSATAASSNDNT | 0.67 | −0.68 | 1.27 |
| 2251 | ATLVT012XX3210 | 48144 | TSQAGGSSNDNT | −0.22 | −0.47 | 2.52 |
| 2252 | ATLVT012XX791 | 45725 | NSESAGSTNDNH | 0.1 | 1.12 | −0.08 |
| 2253 | ATLVT012XX793 | 45727 | NSESGAASNDNA | 0.14 | −0.33 | 1.71 |
| 2254 | ATLVT012XX3000 | 47934 | TSATGAASNDNI | 0.48 | 0.2 | 0.44 |
| 2255 | ATLVT012XX389 | 45323 | NGQTAAATNDNH | 0.08 | −0.05 | 1.44 |
| 2256 | ATLVT012XX272 | 45206 | NGETGASSNDNH | −0.61 | 0.67 | 1.69 |
| 2257 | ATLVT012XX255 | 45189 | NGETAGASNDNT | −1.18 | 3.38 | −0.8 |
| 2258 | ATLVT012XX1588 | 46522 | SGTAAASSNDNA | 0.73 | −1.09 | 1.66 |
| 2259 | ATLVT012XX2595 | 47529 | TGQAAAATNDNT | −1.07 | −0.78 | 4.34 |
| 2260 | ATLVT012XX306 | 45240 | NGQAAGAINDNT | 0.06 | 0.33 | 0.98 |
| 2261 | ATLVT012XX846 | 45780 | NSETGAATNDNT | 0.81 | −0.48 | 0.73 |
| 2262 | ATLVT012XX1922 | 46856 | SSESAAATNDNH | 1.37 | 0.17 | −1.08 |
| 2263 | ATLVT012XX2959 | 47893 | TSASGASTNDNA | 0.19 | −0.22 | 1.45 |
| 2264 | ATLVT012XX2841 | 47775 | TGTTAASTNQNT | 0.31 | 0.39 | 0.44 |
| 2265 | ATLVT012XX2642 | 47576 | TGQSAAATNDNH | −0.48 | 1.82 | −0.02 |
| 2266 | ATLVT012XX3044 | 47978 | TSEAAGSTNDNH | −0.51 | 0.83 | 1.28 |
| 2267 | ATLVT012XX3194 | 48128 | TSQAGAATNDNH | −0.44 | 0.78 | 1.23 |
| 2268 | ATLVT012XX2445 | 47379 | TGATGGSINDNT | −1.34 | 1.21 | 2.22 |
| 2269 | ATLVT012XX3061 | 47995 | TSEAGGATNDNA | −0.25 | 0.56 | 1.18 |
| 2270 | ATLVT012XX187 | 45121 | NGEAGGSSNDNA | 0.17 | 0.95 | −0.07 |
| 2271 | ATLVT012XX2474 | 47408 | TGEAGAATNDNH | −0.36 | 1.29 | 0.42 |
| 2272 | ATLVT012XX1313 | 46247 | SGEAAGSSNDNH | −0.2 | 0.75 | 0.83 |
| 2273 | ATLVT012XX3272 | 48206 | ISQTAASTNDNH | −0.05 | 0.91 | 0.37 |
| 2274 | ATLVT012XX445 | 45379 | NGTAAGASNDNA | −0.27 | 0.39 | 1.4 |
| 2275 | ATLVT012XX448 | 45382 | NGTAAGATNDNA | 0.48 | 0.46 | 0.03 |
| 2276 | ATLVT012XX60 | 44994 | NGASAASINDNT | 2.63 | −1.68 | −0.91 |
| 2277 | ATLVT012XX2305 | 47239 | TGAAAAATNDNA | −0.51 | 0.64 | 1.48 |
| 2278 | ATLVT012XX2177 | 47111 | SSTAAGSSNDNH | −0.3 | 0.66 | 1.09 |
| 2279 | ATLVT012XX2741 | 47675 | TGTAAASSNDNH | −0.66 | 1.16 | 1.06 |
| 2280 | ATLVT012XX832 | 45766 | NSETAGAINDNA | −0.33 | 1.54 | 0.01 |
| 2281 | ATLVT012XX586 | 45520 | NSAAAASTNDNA | −1.68 | 1.59 | 2.24 |
| 2282 | ATLVT012XX1067 | 46001 | NSTSAASTNDNH | −0.08 | 1.54 | −0.43 |
| 2283 | ATLVT012XX1062 | 45996 | NSTSAAATNDNT | −0.64 | −0.02 | 2.53 |
| 2284 | ATLVT012XX2538 | 47472 | TGESGGSSNDNT | −1.36 | 0.14 | 3.55 |
| 2285 | ATLVT012XX2703 | 47637 | TGQTAGAINDNT | 0.25 | −0.22 | 1.25 |
| 2286 | ATLVT012XX76 | 45010 | NGASGAATNDNA | 0.46 | 0.12 | 0.45 |
| 2287 | ATLVT012XX1981 | 46915 | SSETAGATNDNA | −0.19 | 1.05 | 0.37 |
| 2288 | ATLVT012XX757 | 45691 | NSEAGGASNDNA | −1.21 | 1.27 | 1.82 |
| 2289 | ATLVT012XX437 | 45371 | NGTAAAATNDNH | −1.54 | 0.36 | 3.56 |
| 2290 | ATLVT012XX450 | 45384 | NGTAAGAINDNT | 0.58 | −0.26 | 0.72 |
| 2291 | ATLVT012XX2421 | 47355 | TGATAGSINDNT | −0.71 | 0.37 | 2.12 |
| 2292 | ATLVT012XX277 | 45211 | NGETGGASNDNA | 1 | −0.43 | 0.22 |
| 2293 | ATLVT012XX2385 | 47319 | TGASGASTNDNT | −0.15 | 1.03 | 0.29 |
| 2294 | ATLVT012XX756 | 45690 | NSEAGASINDNT | 0.67 | −0.47 | 0.82 |
| 2295 | ATLVT012XX3199 | 48133 | TSQAGASINDNA | −1.14 | 2.52 | 0.07 |
| 2296 | ATLVT012XX1199 | 46133 | SGASAAASNDNH | 0.65 | −0.09 | 0.37 |
| 2297 | ATLVT012XX463 | 45397 | NGTAGASSNDNA | 0.46 | −0.13 | 0.74 |
| 2298 | ATLVT012XX2370 | 47304 | TGASAGSSNDNT | −0.72 | 1.09 | 1.19 |
| 2299 | ATLVT012XX2212 | 47146 | SSTSAASSNDNA | 0.25 | 0.57 | 0.2 |
| 2300 | ATLVT012XX2173 | 47107 | SSTAAGATNDNA | −0.31 | 0.44 | 1.31 |
| 2301 | ATLVT012XX2465 | 47399 | TGEAAGSSNDNH | −0.4 | 1.44 | 0.17 |
| 2302 | ATLVT012XX2085 | 47019 | SSQSAGSTNDNT | −0.36 | 1.65 | −0.18 |
| 2303 | ATLVT012XX3263 | 48197 | TSQTAAASNDNH | 0.75 | −1 | 1.33 |
| 2304 | ATLVT012XX2832 | 47766 | TGTTAAASNDNT | 0.74 | −0.03 | 0.09 |
| 2305 | ATLVT012XX2462 | 47396 | TGEAAGAINDNH | −0.4 | 0.42 | 1.46 |
| 2306 | ATLVT012XX2017 | 46951 | SSQAAAATNDNA | −2.35 | −1.21 | 6.89 |
| 2307 | ATLVT012XX678 | 45612 | NSATAAATNDNT | 1.14 | −0.92 | 0.53 |
| 2308 | ATLVT012XX2884 | 47818 | TSAAAASSNDNA | −0.88 | 1.54 | 0.83 |
| 2309 | ATLVT012XX2364 | 47298 | TGASAGASNDNT | 0.46 | 0 | 0.51 |
| 2310 | ATLVT012XX3063 | 47997 | TSEAGGATNDNT | 0.27 | −0.01 | 0.86 |
| 2311 | ATLVT012XX351 | 45285 | NGQSAGASNDNT | −0.27 | 0.02 | 1.73 |
| 2312 | ATLVT012XX1328 | 46262 | SGEAGASTNDNH | 1.25 | 0.04 | −0.91 |
| 2313 | ATLVT012XX366 | 45300 | NGQSGAATNDNT | 0.72 | −0.54 | 0.76 |
| 2314 | ATLVT012XX1366 | 46300 | SGESGAASNDNA | −0.63 | 1.63 | 0.28 |
| 2315 | ATLVT012XX1322 | 46256 | SGEAGAATNDNH | 0.05 | 0.61 | 0.42 |
| 2316 | ATLVT012XX3288 | 48222 | TSQTGAASNDNT | 0.26 | 0.15 | 0.65 |
| 2317 | ATLVT012XX3215 | 48149 | TSQSAAASNDNH | −0.04 | −0.07 | 1.44 |
| 2318 | ATLVT012XX1639 | 46573 | SGTSAASTNDNA | 0.04 | 0.17 | 1 |
| 2319 | ATLVT012XX1756 | 46690 | SSAAGASSNDNA | 0.14 | 0.24 | 0.74 |
| 2320 | ATLVT012XX2662 | 47596 | TGQSGAASNDNA | −0.41 | 0.61 | 1.19 |
| 2321 | ATLVT012XX3190 | 48124 | TSQAGAASNDNA | −0.75 | 1.26 | 0.94 |
| 2322 | ATLVT012XX628 | 45562 | NSASAAATNDNA | 1.93 | −0.57 | −1.31 |
| 2323 | ATLVT012XX1839 | 46773 | SSATAGATNDNT | −0.11 | 0.96 | 0.23 |
| 2324 | ATLVT012XX177 | 45111 | NGEAGASSNDNT | 0.37 | 1.26 | −0.98 |
| 2325 | ATLVT012XX124 | 45058 | NGATGAATNDNA | −0.1 | 0.67 | 0.56 |
| 2326 | ATLVT012XX107 | 45041 | NGATAASTNDNH | 0.13 | 1.49 | −0.89 |
| 2327 | ATLVT012XX1853 | 46787 | SSATGASSNDNH | −0.51 | 2.85 | −1.55 |
| 2328 | ATLVT012XX693 | 45627 | NSATAGSSNDNT | −0.35 | 0.39 | 1.33 |
| 2329 | ATLVT012XX1510 | 46444 | SGQSGAASNDNA | −0.08 | −0.19 | 1.61 |
| 2330 | ATLVT012XX1159 | 46093 | SGAAAASINDNA | −1.09 | 1.19 | 1.58 |
| 2331 | ATLVT012XX1594 | 46528 | SGTAAGASNDNA | −0.26 | 1.02 | 0.35 |
| 2332 | ATLVT012XX1749 | 46683 | SSAAAGSINDNT | −0.38 | 0.42 | 1.32 |
| 2333 | ATLVT012XX1343 | 46277 | SGESAAASNDNH | 1.71 | −1.48 | 0.18 |
| 2334 | ATLVT012XX1997 | 46931 | SSETGASSNDNH | 1.38 | −0.32 | −0.75 |
| 2335 | ATLVT012XX1349 | 46283 | SGESAASSNDNH | −0.23 | 0.8 | 0.53 |
| 2336 | ATLVT012XX1402 | 46336 | SGETAGASNDNA | 0.13 | 0.65 | 0.14 |
| 2337 | ATLVT012XX3414 | 48348 | TSTTAASSNDNT | −0.44 | 0.14 | 1.77 |
| 2338 | ATLVT012XX260 | 45194 | NGETAGSSNDNH | 0.73 | 0.82 | −1.1 |
| 2339 | ATLVT012XX1452 | 46386 | SGQAAGASNDNT | −0.72 | 1.22 | 0.86 |
| 2340 | ATLVT012XX49 | 44983 | NGASAAASNDNA | 0.39 | 0.34 | 0.08 |
| 2341 | ATLVT012XX1760 | 46694 | SSAAGASINDNH | 0.1 | 0.23 | 0.72 |
| 2342 | ATLVT012XX537 | 45471 | NGTTAASSNDNT | −0.82 | 0.92 | 1.39 |
| 2343 | ATLVT012XX2669 | 47603 | TGQSGASSNDNH | −0.94 | 1.46 | 0.9 |
| 2344 | ATLVT012XX2940 | 47874 | TSASAGASNDNT | −0.46 | 0.55 | 1.24 |
| 2345 | ATLVT012XX977 | 45911 | NSQTAGATNDNH | 0.92 | −0.4 | 0.08 |
| 2346 | ATLVT012XX1539 | 46473 | SGQTAAATNDNT | 0.89 | −1.08 | 1 |
| 2347 | ATLVT012XX1926 | 46860 | SSESAASSNDNT | −0.3 | −1.2 | 3.2 |
| 2348 | ATLVT012XX2263 | 47197 | SSTTAASTNDNA | −0.69 | 0.84 | 1.25 |
| 2349 | ATLVT012XX212 | 45146 | NGESAGSSNDNH | 0.74 | −0.13 | 0.02 |
| 2350 | ATLVT012XX584 | 45518 | NSAAAASSNDNH | −0.37 | 1.16 | 0.27 |
| 2351 | ATLVT012XX2608 | 47542 | TGQAAGSSNDNA | −0.65 | 0.35 | 1.79 |
| 2352 | ATLVT012XX3247 | 48181 | TSQSGASTNDNA | −0.7 | 0.67 | 1.45 |
| 2353 | ATLVT012XX3220 | 48154 | TSQSAASSNDNA | 1.45 | −0.68 | 0.5 |
| 2354 | ATLVT012XX629 | 45563 | NSASAAATNDNH | 1.24 | −0.47 | −0.4 |
| 2355 | ATLVT012XX197 | 45131 | NGESAAATNDNH | −1.42 | 0.76 | 2.58 |
| 2356 | ATLVT012XX31 | 44965 | NGAAGASSNDNA | −0.26 | 0.35 | 1.1 |
| 2357 | ATLVT012XX2133 | 47067 | SSQTAGSINDNT | 0.08 | −0.4 | 1.49 |
| 2358 | ATLVT012XX2398 | 47332 | TGATAAASNDNA | −1.22 | 2.58 | −0.12 |
| 2359 | ATLVT012XX2503 | 47437 | TGESAASTNDNA | −1.15 | 2.58 | −0.27 |
| 2360 | ATLVT012XX2570 | 47504 | TGETGAATNDNH | −0.1 | 0.23 | 0.97 |
| 2361 | ATLVT012XX616 | 45550 | NSAAGGATNDNA | −0.69 | 1.13 | 0.82 |
| 2362 | ATLVT012XX3104 | 48038 | TSESGASTNDNH | 0.42 | 0.04 | 0.32 |
| 2363 | ATLVT012XX1298 | 46232 | SGEAAAATNDNH | −0.94 | −0.47 | 3.29 |
| 2364 | ATLVT012XX3415 | 48349 | TSTTAASINDNA | −0.7 | −0.04 | 2.33 |
| 2365 | ATLVT012XX1164 | 46098 | SGAAAGASNDNT | −0.32 | 0.45 | 1.05 |
| 2366 | ATLVT012XX2086 | 47020 | SSQSGAASNDNA | 0 | −0.08 | 1.19 |
| 2367 | ATLVT012XX1357 | 46291 | SGESAGAINDNA | 2.05 | −1.43 | −0.61 |
| 2368 | ATLVT012XX2471 | 47405 | TGEAGAASNDNH | −0.36 | 0.83 | 0.61 |
| 2369 | ATLVT012XX898 | 45832 | NSQAGASTNDNA | −0.54 | 0.93 | 0.8 |
| 2370 | ATLVT012XX22 | 44956 | NGAAAGSINDNA | −0.14 | 0.24 | 0.99 |
| 2371 | ATLVT012XX2399 | 47333 | TGATAAASNDNH | −0.02 | 0.33 | 0.67 |
| 2372 | ATLVT012XX1614 | 46548 | SGTAGASSNDNT | −1.12 | 0.72 | 2.04 |
| 2373 | ATLVT012XX62 | 44996 | NGASAGASNDNH | 0.26 | 0.15 | 0.42 |
| 2374 | ATLVT012XX1545 | 46479 | SGQTAASTNDNT | 0.67 | −0.63 | 0.71 |
| 2375 | ATLVT012XX3271 | 48205 | TSQTAASTNDNA | −0.1 | −0.01 | 1.22 |
| 2376 | ATLVT012XX2044 | 46978 | SSQAGASSNDNA | 0.74 | −0.69 | 0.66 |
| 2377 | ATLVT012XX2172 | 47106 | SSTAAGASNDNT | −0.64 | 0.27 | 1.78 |
| 2378 | ATLVT012XX2659 | 47593 | TGQSAGSTNDNA | 0.04 | 1.11 | −0.45 |
| 2379 | ATLVT012XX1786 | 46720 | SSASAGASNDNA | 0.63 | 0.3 | −0.42 |
| 2380 | ATLVT012XX2469 | 47403 | TGEAAGSINDNT | −0.25 | 0.72 | 0.54 |
| 2381 | ATLVT012XX3052 | 47986 | TSEAGASSNDNA | 0.2 | 0.23 | 0.38 |
| 2382 | ATLVT012XX2693 | 47627 | TGQTAASSNDNH | 0.24 | −0.38 | 1.11 |
| 2383 | ATLVT012XX1303 | 46237 | SGEAAASTNDNA | 2.69 | −2.01 | −1 |
| 2384 | ATLVT012XX2938 | 47872 | TSASAGASNDNA | 0.28 | 0.44 | −0.01 |
| 2385 | ATLVT012XX2277 | 47211 | SSTTAGSTNDNT | −0.49 | 1.26 | 0.26 |
| 2386 | ATLVT012XX3024 | 47958 | TSEAAAASNDNT | 1.8 | −1.23 | −0.47 |
| 2387 | ATLVT012XX285 | 45219 | NGETGGSSNDNT | −1.74 | −0.44 | 4.57 |
| 2388 | ATLVT012XX3059 | 47993 | TSEAGGASNDNH | −0.57 | 0.35 | 1.55 |
| 2389 | ATLVT012XX3244 | 48178 | TSQSGASSNDNA | −0.81 | 1.42 | 0.57 |
| 2390 | ATLVT012XX3 | 44937 | NGAAAAASNDNT | −0.12 | 0.2 | 0.97 |
| 2391 | ATLVT012XX1258 | 46192 | SGATAGASNDNA | 0.62 | −0.2 | 0.2 |
| 2392 | ATLVT012XX967 | 45901 | NSQTAASSNDNA | 0.34 | −1.06 | 1.77 |
| 2393 | ATLVT012XX541 | 45475 | NGTTAGASNDNA | −0.47 | 0.36 | 1.34 |
| 2394 | ATLVT012XX743 | 45677 | NSEAAGSINDNH | −1.24 | 2.58 | −0.21 |
| 2395 | ATLVT012XX1905 | 46839 | SSEAGASTNQNT | 1.1 | −0.86 | 0.22 |
| 2396 | ATLVT012XX298 | 45232 | NGQAAASTNDNA | 0.64 | −0.87 | 1 |
| 2397 | ATLVT012XX2323 | 47257 | TGAAAGSINDNA | −0.38 | 0.38 | 1.14 |
| 2398 | ATLVT012XX99 | 45033 | NGATAAASNDNT | 0.56 | 0.42 | −0.51 |
| 2399 | ATLVT012XX2274 | 47208 | SSTTAGSSNDNT | −1.3 | 2.3 | 0.25 |
| 2400 | ATLVT012XX1408 | 46342 | SGETAGSSNDNA | −0.15 | 1.6 | −0.83 |
| 2401 | ATLVT012XX451 | 45385 | NGTAAGSSNDNA | 0.61 | −0.06 | 0.01 |
| 2402 | ATLVT012XX2129 | 47063 | SSQTAGSSNDNH | 0.18 | 0.17 | 0.44 |
| 2403 | ATLVT012XX599 | 45533 | NSAAAGSINDNH | −0.73 | 0.89 | 1.08 |
| 2404 | ATLVT012XX2692 | 47626 | TGQTAASSNDNA | 0.07 | −0.24 | 1.15 |
| 2405 | ATLVT012XX2268 | 47202 | SSTTAGASNDNT | −0.17 | 0.84 | 0.17 |
| 2406 | ATLVT012XX2835 | 47769 | TGTTAAATNDNT | −0.53 | 0.63 | 1.04 |
| 2407 | ATLVT012XX2545 | 47479 | TGETAAATNDNA | 1.2 | −0.52 | −0.44 |
| 2408 | ATLVT012XX1198 | 46132 | SGASAAASNDNA | −0.31 | 1.73 | −0.74 |
| 2409 | ATLVT012XX1416 | 46350 | SGETGAASNDNT | 0.16 | −0.07 | 0.75 |
| 2410 | ATLVT012XX2573 | 47507 | TGETGASSNDNH | 0.64 | −0.2 | 0.08 |
| 2411 | ATLVT012XX1257 | 46191 | SGATAASTNDNT | 0.82 | 0.38 | −0.99 |
| 2412 | ATLVT012XX1272 | 46206 | SGATGAASNDNT | 0.39 | 0.28 | −0.14 |
| 2413 | ATLVT012XX3143 | 48077 | TSETGAASNDNH | −0.38 | 0.34 | 1.1 |
| 2414 | ATLVT012XX2 | 44936 | NGAAAAASNDNH | −0.49 | 0.5 | 1.08 |
| 2415 | ATLVT012XX2404 | 47338 | TGATAASSNDNA | −0.16 | 0.33 | 0.73 |
| 2416 | ATLVT012XX1683 | 46617 | SGTTAAATNDNT | 0.26 | −0.23 | 0.74 |
| 2417 | ATLVT012XX654 | 45588 | NSASGAATNDNT | 0.2 | −0.13 | 0.7 |
| 2418 | ATLVT012XX1686 | 46620 | SGTTAASSNDNT | −0.72 | 0.11 | 1.96 |
| 2419 | ATLVT012XX1927 | 46861 | SSESAASTNDNA | 0.15 | 1.14 | −0.85 |
| 2420 | ATLVT012XX2079 | 47013 | SSQSAGATNDNT | 0.22 | 0.02 | 0.48 |
| 2421 | ATLVT012XX2862 | 47796 | TGTTGASSNDNT | −0.18 | 0.36 | 0.7 |
| 2422 | ATLVT012XX1273 | 46207 | SGATGAATNDNA | 0.4 | 0.02 | 0.14 |
| 2423 | ATLVT012XX2611 | 47545 | TGQAAGSINDNA | −0.81 | 0.08 | 2.13 |
| 2424 | ATLVT012XX1977 | 46911 | SSETAASINDNT | 0.58 | −1.61 | 1.93 |
| 2425 | ATLVT012XX2668 | 47602 | TGQSGASSNDNA | −0.55 | 0.42 | 1.25 |
| 2426 | ATLVT012XX2594 | 47528 | TGQAAAATNDNH | −0.95 | 0.01 | 2.46 |
| 2427 | ATLVT012XX3427 | 48361 | TSTTAGSINDNA | −0.51 | 0.33 | 1.29 |
| 2428 | ATLVT012XX1986 | 46920 | SSETAGSSNDNT | 1.44 | −0.82 | −0.57 |
| 2429 | ATLVT012XX660 | 45594 | NSASGASINDNT | 0.17 | 0.31 | 0.14 |
| 2430 | ATLVT012XX2986 | 47920 | TSATAGASNDNA | −0.79 | 0.5 | 1.53 |
| 2431 | ATLVT012XX33 | 44967 | NGAAGASSNDNI | −0.77 | 0.51 | 1.48 |
| 2432 | ATLVT012XX2709 | 47643 | TGQTAGSINDNT | −0.43 | 0.41 | 1.01 |
| 2433 | ATLVT012XX2536 | 47470 | TGESGGSSNDNA | −0.85 | −0.04 | 2.3 |
| 2434 | ATLVT012XX553 | 45487 | NGTTGAASNDNA | −0.07 | −0.19 | 1.16 |
| 2435 | ATLVT012XX585 | 45519 | NSAAAASSNDN'T | −0.59 | 0.72 | 0.89 |
| 2436 | ATLVT012XX2860 | 47794 | TGTTGASSNDNA | −0.73 | 1.59 | 0.01 |
| 2437 | ATLVT012XX267 | 47605 | TGQSGASTNDNA | −1.32 | 1.19 | 1.51 |
| 2438 | ATLVT012XX3088 | 48022 | TSESAGSSNDNA | −2.89 | 0.71 | 4.84 |
| 2439 | ATLVT012XX3273 | 48207 | TSQTAASINDNT | 0.07 | −0.48 | 1.29 |
| 2440 | ATLVT012XX1969 | 46903 | SSETAAATNDNA | −0.73 | 0.35 | 1.58 |
| 2441 | ATLVT012XX3159 | 48093 | TSETGGATNDNT | 0.07 | 0.79 | −0.34 |
| 2442 | ATLVT012XX2142 | 47076 | SSQTGASSNDNT | −0.89 | −0.41 | 2.84 |
| 2443 | ATLVT012XX1845 | 46779 | SSATAGSINDNT | 0.03 | 0.49 | 0.09 |
| 2444 | ATLVT012XX1828 | 46762 | SSATAASSNDNA | 0.58 | −0.77 | 0.76 |
| 2445 | ATLVT012XX368 | 45302 | NGQSGASSNDNH | −0.14 | 0.44 | 0.46 |
| 2446 | ATLVT012XX1227 | 46161 | SGASGAATNDNT | 0.59 | −0.9 | 0.92 |
| 2447 | ATLVT012XX2381 | 47315 | TGASGASSNDNH | −0.38 | 0.05 | 1.36 |
| 2448 | ATLVT012XX2179 | 47113 | SSTAAGSINDNA | −0.55 | 0.83 | 0.63 |
| 2449 | ATLVT012XX2558 | 47492 | TGETAGATNDNH | 0.63 | 0.28 | −0.69 |
| 2450 | ATLVT012XX659 | 45593 | NSASGASTNDNH | −0.13 | 1.05 | −0.37 |
| 2451 | ATLVT012XX1633 | 46567 | SGTSAAATNDNA | −0.34 | −0.03 | 1.37 |
| 2452 | ATLVT012XX789 | 45723 | NSESAGSSNDNT | −0.26 | 0.16 | 0.99 |
| 2453 | ATLVT012XX2667 | 47601 | TGQSGAATNDNT | −0.81 | 0.94 | 0.92 |
| 2454 | ATLVT012XX3419 | 48353 | TSTTAGASNDNH | −0.32 | 0.1 | 1.15 |
| 2455 | ATLVT012XX1758 | 46692 | SSAAGASSNDNT | −0.52 | −0.06 | 1.71 |
| 2456 | ATLVT012XX440 | 45374 | NGTAAASSNDNH | −1.49 | 1.96 | 0.76 |
| 2457 | ATLVT012XX2513 | 47447 | TGESAGSSNDNH | −0.64 | 1.26 | 0.2 |
| 2458 | ATLVT012XX372 | 45306 | NGQSGASINDNT | 0.15 | −0.57 | 1.2 |
| 2459 | ATLVT012XX66 | 45000 | NGASAGATNDNT | −0.44 | 0.21 | 1.21 |
| 2460 | ATLVT012XX934 | 45868 | NSQSAGSINDNA | 0.59 | 0.28 | −0.66 |
| 2461 | ATLVT012XX1205 | 46139 | SGASAASSNDNH | −0.03 | −0.21 | 1.03 |
| 2462 | ATLVT012XX3192 | 48126 | TSQAGAASNDNT | −0.64 | 0.65 | 0.97 |
| 2463 | ATLVT012XX111 | 45045 | NGATAGASNDNT | 0.49 | 0.04 | −0.19 |
| 2464 | ATLVT012XX2470 | 47404 | TGEAGAASNDNA | −1.15 | 1.73 | 0.45 |
| 2465 | ATLVT012XX1582 | 46516 | SGTAAAASNDNA | −0.29 | 0.35 | 0.75 |
| 2466 | ATLVT012XX3430 | 48364 | TSTTGAASNDNA | 0.3 | −0.34 | 0.63 |
| 2467 | ATLVT012XX779 | 45713 | NSESAASTNDNH | 0.04 | 1.66 | −1.51 |
| 2468 | ATLVT012XX733 | 45667 | NSEAAGASNDNA | −0.64 | 0.68 | 0.9 |
| 2469 | ATLVT012XX269 | 45203 | NGETGAATNDNH | −1.6 | −0.21 | 3.69 |
| 2470 | ATLVT012XX293 | 45227 | NGQAAAATNDNH | 0.98 | −1.56 | 1 |
| 2471 | ATLVT012XX1798 | 46732 | SSASGAASNDNA | 0.31 | −0.15 | 0.33 |
| 2472 | ATLVT012XX2656 | 47590 | TGQSAGSSNDNA | 0.67 | −0.06 | −0.41 |
| 2473 | ATLVT012XX1494 | 46428 | SGQSAASSNDNT | −0.37 | 0.32 | 0.9 |
| 2474 | ATLVT012XX1935 | 46869 | SSESAGAINDNT | −0.66 | 1.56 | −0.2 |
| 2475 | ATLVT012XX2062 | 46996 | SSQSAAASNDNA | 0.23 | −0.91 | 1.46 |
| 2476 | ATLVT012XX1256 | 46190 | SGATAASTNDNH | 0.33 | 0.74 | −0.85 |
| 2477 | ATLVT012XX1375 | 46309 | SGESGASTNDNA | −0.15 | 0.86 | −0.2 |
| 2478 | ATLVT012XX1493 | 46427 | SGQSAASSNDNH | −0.25 | 0.51 | 0.43 |
| 2479 | ATLVT012XX1391 | 46325 | SGETAAASNDNH | 0.05 | 0.49 | −0.07 |
| 2480 | ATLVT012XX969 | 45903 | NSQTAASSNDNT | −0.29 | −0.93 | 2.33 |
| 2481 | ATLVT012XX2275 | 47209 | SSTTAGSTNDNA | 0.05 | −0.14 | 0.74 |
| 2482 | ATLVT012XX926 | 45860 | NSQSAGASNDNH | −0.04 | 0.21 | 0.43 |
| 2483 | ATLVT012XX876 | 45810 | NSQAAASINDNT | −0.05 | 0.4 | 0.2 |
| 2484 | ATLVT012XX2541 | 47475 | TGESGGSTNDNT | −1.16 | −0.14 | 2.79 |
| 2485 | ATLVT012XX314 | 45248 | NGQAGAASNDNH | 1.02 | −1.01 | 0.17 |
| 2486 | ATLVT012XX1011 | 45945 | NSTAAAASNDNT | −0.33 | −0.42 | 1.71 |
| 2487 | ATLVT012XX359 | 45293 | NGQSAGSTNDNH | −0.61 | 1.6 | −0.43 |
| 2488 | ATLVT012XX790 | 45724 | NSESAGSINDNA | −0.47 | 0.42 | 0.85 |
| 2489 | ATLVT012XX1207 | 46141 | SGASAASTNDNA | 0.15 | 0.9 | −0.84 |
| 2490 | ATLVT012XX16 | 44950 | NGAAAGAINDNA | −0.73 | 0.47 | 1.22 |
| 2491 | ATLVT012XX312 | 45246 | NGQAAGSINDNT | 0.03 | 0.01 | 0.51 |
| 2492 | ATLVT012XX3173 | 48107 | TSQAAASSNDNH | −1.1 | 0.04 | 2.4 |
| 2493 | ATLVT012XX2631 | 47565 | TGQAGGATNDNT | −0.5 | −0.31 | 1.82 |
| 2494 | ATLVT012XX626 | 45560 | NSASAAASNDNH | 0.55 | −1.05 | 0.97 |
| 2495 | ATLVT012XX1462 | 46396 | SGQAGAASNDNA | −0.39 | 0.03 | 1.19 |
| 2496 | ATLVT012XX3360 | 48294 | TSTSAAASNDNT | −1.61 | 0.95 | 2.08 |
| 2497 | ATLVT012XX2498 | 47432 | TGESAAATNDNH | −0.6 | 1.03 | 0.26 |
| 2498 | ATLVT012XX2622 | 47556 | TGQAGASSNDNT | −0.18 | −0.49 | 1.49 |
| 2499 | ATLVT012XX1784 | 46718 | SSASAASTNDNH | −0.34 | 1.11 | −0.31 |
| 2500 | ATLVT012XX3117 | 48051 | TSESGGSINDNT | −0.13 | 0.12 | 0.6 |
| 2501 | ATLVT012XX53 | 44987 | NGASAAATNDNH | −0.55 | 1.05 | 0.12 |
| 2502 | ATLVT012XX1401 | 46335 | SGETAASTNDNT | 0.76 | −0.59 | 0 |
| 2503 | ATLVT012XX1315 | 46249 | SGEAAGSINDNA | −0.36 | −0.74 | 2.09 |
| 2504 | ATLVT012XX2512 | 47446 | TGESAGSSNDNA | −0.98 | 1.61 | 0.12 |
| 2505 | ATLVT012XX2647 | 47581 | TGQSAASTNDNA | −0.82 | 0.93 | 0.73 |
| 2506 | ATLVT012XX3148 | 48082 | TSETGASSNDNA | 1.19 | −1.26 | 0 |
| 2507 | ATLVT012XX1993 | 46927 | SSETGAATNDNA | −0.01 | −0.08 | 0.62 |
| 2508 | ATLVT012XX601 | 45535 | NSAAGAASNDNA | −0.45 | 0.31 | 0.88 |
| 2509 | ATLVT012XX3053 | 47987 | TSEAGASSNDNH | −0.02 | 0.14 | 0.35 |
| 2510 | ATLVT012XX827 | 45761 | NSETAASTNDNH | 0.3 | 0.64 | −0.83 |
| 2511 | ATLVT012XX1590 | 46524 | SGTAAASSNDNT | −0.92 | −0.83 | 3.15 |
| 2512 | ATLVT012XX1359 | 46293 | SGESAGATNDNT | −0.02 | 0.22 | 0.25 |
| 2513 | ATLVT012XX2362 | 47296 | TGASAGASNDNA | −0.6 | 0.4 | 1.01 |
| 2514 | ATLVT012XX2484 | 47418 | TGEAGGASNQNT | 0.09 | 0.23 | 0.05 |
| 2515 | ATLVT012XX818 | 45752 | NSETAAASNDNH | −1.72 | 1.21 | 1.87 |
| 2516 | ATLVT012XX412 | 45346 | NGQTGAATNDNA | −1.05 | −0.08 | 2.38 |
| 2517 | ATLVT012XX3407 | 48341 | TSTTAAASNDNH | −0.62 | 0.88 | 0.4 |
| 2518 | ATLVT012XX2734 | 47668 | TGTAAAASNDNA | −1.02 | 1.13 | 0.77 |
| 2519 | ATLVT012XX982 | 45916 | NSQTAGSINDNA | 1.37 | −1.12 | −0.46 |
| 2520 | ATLVT012XX696 | 45630 | NSATAGSTNDNT | −1.46 | 0.02 | 2.93 |
| 2521 | ATLVT012XX1933 | 46867 | SSESAGATNDNA | −0.85 | 1.23 | 0.31 |
| 2522 | ATLVT012XX1204 | 46138 | SGASAASSNDNA | 0.04 | 0.16 | 0.17 |
| 2523 | ATLVT012XX2599 | 47533 | TGQAAASTNDNA | −1.14 | 1.43 | 0.56 |
| 2524 | ATLVT012XX1202 | 46136 | SGASAAATNDNH | 0.11 | 0.45 | −0.32 |
| 2525 | ATLVT012XX279 | 45213 | NGETGGASNDNT | −1.68 | 1.99 | 0.74 |
| 2526 | ATLVT012XX2021 | 46955 | SSQAAASSNDNH | −1.39 | 0.9 | 1.64 |
| 2527 | ATLVT012XX2110 | 47044 | SSQTAAASNDNA | −1.52 | 2.05 | 0.39 |
| 2528 | ATLVT012XX1456 | 46390 | SGQAAGSSNDNA | −0.27 | 0.56 | 0.14 |
| 2529 | ATLVT012XX2718 | 47652 | TGQTGASSNDNT | −0.47 | −0.1 | 1.33 |
| 2530 | ATLVT012XX278 | 45212 | NGETGGASNDNH | −0.39 | 0.59 | 0.32 |
| 2531 | ATLVT012XX627 | 45561 | NSASAAASNDNT | 0.82 | −0.87 | 0.12 |
| 2532 | ATLVT012XX2910 | 47844 | TSAAGASSNDNT | −0.57 | 0.42 | 0.84 |
| 2533 | ATLVT012XX2495 | 47429 | TGESAAASNDNH | −1.18 | 1.14 | 0.96 |
| 2534 | ATLVT012XX2931 | 47865 | TSASAAATNDNT | −1.37 | −0.47 | 3.34 |
| 2535 | ATLVT012XX1680 | 46614 | SGTTAAASNDNI | 0.55 | −0.79 | 0.47 |
| 2536 | ATLVT012XX2901 | 47835 | TSAAAGSINDNT | −1.18 | 1.34 | 0.69 |
| 2537 | ATLVT012XX2711 | 47645 | TGQTGAASNDNH | −0.45 | 0.06 | 1.08 |
| 2538 | ATLVT012XX21 | 44955 | NGAAAGSSNDNT | −0.4 | 0.04 | 1.02 |
| 2539 | ATLVT012XX224 | 45158 | NGESGASSNDNH | 0.37 | 0.22 | −0.53 |
| 2540 | ATLVT012XX1248 | 46182 | SGATAAASNDNT | 1.4 | −0.74 | −1.06 |
| 2541 | ATLVT012XX2833 | 47767 | TGTTAAATNDNA | −0.76 | 0.87 | 0.56 |
| 2542 | ATLVT012XX2020 | 46954 | SSQAAASSNDNA | −1.55 | 3.23 | −1.12 |
| 2543 | ATLVT012XX942 | 45876 | NSQSGAATNDNT | 0.57 | −0.48 | 0.01 |
| 2544 | ATLVT012XX2640 | 47574 | TGQSAAASNDNT | −0.48 | −0.16 | 1.39 |
| 2545 | ATLVT012XX3216 | 48150 | TSQSAAASNDNT | 0.3 | −0.65 | 0.68 |
| 2546 | ATLVT012XX1425 | 46359 | SGETGASTNDNT | 0.96 | −1.51 | 0.64 |
| 2547 | ATLVT012XX3225 | 48159 | TSQSAASTNDNT | 0.34 | −0.35 | 0.2 |
| 2548 | ATLVT012XX3008 | 47942 | TSATGASINDNH | 0.06 | 0.47 | −0.37 |
| 2549 | ATLVT012XX439 | 45373 | NGTAAASSNDNA | −1.63 | 1.85 | 0.74 |
| 2550 | ATLVT012XX799 | 45733 | NSESGASSNDNA | 0.98 | −1.15 | 0.14 |
| 2551 | ATLVT012XX1352 | 46286 | SGESAASTNDNH | −0.47 | 0.9 | −0.04 |
| 2552 | ATLVT012XX15 | 44949 | NGAAAGASNDNT | −0.86 | 0.21 | 1.53 |
| 2553 | ATLVT012XX289 | 45223 | NGQAAAASNDNA | 0.77 | −1.54 | 0.99 |
| 2554 | ATLVT012XX2899 | 47833 | TSAAAGSINDNA | −0.94 | 0.8 | 0.9 |
| 2555 | ATLVT012XX895 | 45829 | NSQAGASSNDNA | −0.15 | 0.24 | 0.26 |
| 2556 | ATLVT012XX1311 | 46245 | SGEAAGAINDNT | −0.79 | 0.23 | 1.37 |
| 2557 | ATLVT012XX3079 | 48013 | TSESAASTNDNA | −2.24 | 3.09 | 0.18 |
| 2558 | ATLVT012XX2025 | 46959 | SSQAAASINDNT | 0.37 | −0.76 | 0.64 |
| 2559 | ATLVT012XX2535 | 47469 | TGESGGATNDNT | −1.15 | 0.95 | 1.05 |
| 2560 | ATLVT012XX3089 | 48023 | TSESAGSSNDNH | −1.46 | 0.93 | 1.6 |
| 2561 | ATLVT012XX3077 | 48011 | TSESAASSNDNH | −0.96 | 1.33 | 0.23 |
| 2562 | ATLVT012XX2564 | 47498 | TGETAGSINDNH | −0.08 | 0.68 | −0.44 |
| 2563 | ATLVT012XX1953 | 46887 | SSESGASTNDNT | −0.24 | 0.36 | 0.24 |
| 2564 | ATLVT012XX217 | 45151 | NGESGAASNDNA | 0.69 | −0.71 | 0.02 |
| 2565 | ATLVT012XX2568 | 47502 | TGETGAASNDNT | 0.11 | −0.27 | 0.44 |
| 2566 | ATLVT012XX1945 | 46879 | SSESGAATNDNA | 0.26 | 0.09 | −0.28 |
| 2567 | ATLVT012XX1789 | 46723 | SSASAGAINDNA | 0.6 | 0.27 | −1.09 |
| 2568 | ATLVT012XX1470 | 46404 | SGQAGASSNDNT | −0.48 | −0.35 | 1.54 |
| 2569 | ATLVT012XX3093 | 48027 | TSESAGSTNDNT | −1.52 | 1.19 | 1.36 |
| 2570 | ATLVT012XX3294 | 48228 | TSQTGASSNDNT | −0.48 | 0.73 | 0.15 |
| 2571 | ATLVT012XX705 | 45639 | NSATGASSNDNT | −0.51 | 1.2 | −0.38 |
| 2572 | ATLVT012XX1201 | 46135 | SGASAAATNDNA | −1.1 | −0.73 | 3.1 |
| 2573 | ATLVT012XX1250 | 46184 | SGATAAATNDNH | 0.66 | 0.12 | −1.02 |
| 2574 | ATLVT012XX2981 | 47915 | TSATAASSNDNH | −0.88 | 1.21 | 0.23 |
| 2575 | ATLVT012XX837 | 45771 | NSETAGSSNDNT | −0.63 | 0.23 | 1.05 |
| 2576 | ATLVT012XX3176 | 48110 | TSQAAASTNDNH | −0.08 | −0.25 | 0.72 |
| 2577 | ATLVT012XX3098 | 48032 | TSESGAATNDNH | −0.57 | 0.46 | 0.65 |
| 2578 | ATLVT012XX3418 | 48352 | TSTTAGASNDNA | −0.1 | 0.08 | 0.32 |
| 2579 | ATLVT012XX2652 | 47586 | TGQSAGASNDNT | −0.11 | 0 | 0.43 |
| 2580 | ATLVT012XX3142 | 48076 | TSETGAASNDNA | −0.46 | 0.28 | 0.68 |
| 2581 | ATLVT012XX213 | 45147 | NGESAGSSNDNT | 0.35 | −0.67 | 0.51 |
| 2582 | ATLVT012XX3055 | 47989 | TSEAGASTNDNA | −0.69 | 1.18 | −0.08 |
| 2583 | ATLVT012XX820 | 45754 | NSETAAATNDNA | 1.63 | −1.19 | −1.03 |
| 2584 | ATLVT012XX446 | 45380 | NGTAAGASNDNH | −0.31 | −0.18 | 1.02 |
| 2585 | ATLVT012XX2485 | 47419 | TGEAGGATNDNA | 0.41 | −0.39 | 0.05 |
| 2586 | ATLVT012XX158 | 45092 | NGEAAGASNDNH | −0.4 | −0.3 | 1.31 |
| 2587 | ATLVT012XX112 | 45046 | NGATAGAINDNA | 0.89 | −0.49 | −0.66 |
| 2588 | ATLVT012XX2638 | 47572 | TGQSAAASNDNA | 0.01 | −0.5 | 0.85 |
| 2589 | ATLVT012XX2432 | 47366 | TGATGASTNDNH | 0.03 | 0.04 | 0.11 |
| 2590 | ATLVT012XX2175 | 47109 | SSTAAGAINDNT | −0.16 | 0.07 | 0.41 |
| 2591 | ATLVT012XX3083 | 48017 | TSESAGASNDNH | −1.44 | 0.22 | 2.4 |
| 2592 | ATLVT012XX2509 | 47443 | TGESAGATNDNA | −0.94 | 0.73 | 0.89 |
| 2593 | ATLVT012XX762 | 45696 | NSEAGGATNDNT | −0.85 | 0.93 | 0.48 |
| 2594 | ATLVT012XX2461 | 47395 | TGEAAGATNDNA | −0.82 | 0.29 | 1.24 |
| 2595 | ATLVT012XX915 | 45849 | NSQSAAASNDNT | 0.01 | −0.26 | 0.51 |
| 2596 | ATLVT012XX2357 | 47291 | TGASAASSNDNH | −1.36 | 1.54 | 0.55 |
| 2597 | ATLVT012XX2739 | 47673 | TGTAAAATNDNT | 0.38 | −1.22 | 1.11 |
| 2598 | ATLVT012XX3200 | 48134 | TSQAGASINDNH | −0.75 | 0.95 | 0.23 |
| 2599 | ATLVT012XX2719 | 47653 | TGQTGASTNDNA | −0.08 | −0.13 | 0.47 |
| 2600 | ATLVT012XX1015 | 45949 | NSTAAASSNDNA | 0.42 | −0.39 | −0.05 |
| 2601 | ATLVT012XX2974 | 47908 | TSATAAASNDNA | −0.58 | 1.14 | −0.32 |
| 2602 | ATLVT012XX1469 | 46403 | SGQAGASSNDNH | −1.12 | 0.76 | 1.09 |
| 2603 | ATLVT012XX692 | 45626 | NSATAGSSNDNH | 0.55 | −0.57 | −0.06 |
| 2604 | ATLVT012XX3237 | 48171 | TSQSAGSTNQNT | −0.42 | 0.17 | 0.64 |
| 2605 | ATLVT012XX11 | 44945 | NGAAAASTNINH | −0.51 | 0.41 | 0.48 |
| 2606 | ATLVT012XX219 | 45153 | NGESGAASNDNT | 0.91 | −1.03 | −0.09 |
| 2607 | ATLVT012XX896 | 45830 | NSQAGASSNDNH | −0.44 | 0.71 | −0.02 |
| 2608 | ATLVT012XX1903 | 46837 | SSEAGASTNDNA | 0.43 | −0.46 | 0 |
| 2609 | ATLVT012XX1153 | 46087 | SGAAAAATNDNA | −1.2 | 0.45 | 1.6 |
| 2610 | ATLVT012XX1230 | 46164 | SGASGASSNDNT | −0.58 | 0.77 | 0.13 |
| 2611 | ATLVT012XX68 | 45002 | NGASAGSSNDNH | 1.36 | −0.89 | −1.06 |
| 2612 | ATLVT012XX2863 | 47797 | TGTTGASTNDNA | −0.79 | 0.52 | 0.81 |
| 2613 | ATLVT012XX414 | 45348 | NGQTGAATNDNT | −0.43 | −0.62 | 1.65 |
| 2614 | ATLVT012XX1468 | 46402 | SGQAGASSNDNA | −0.43 | 0.05 | 0.79 |
| 2615 | ATLVT012XX1150 | 46084 | SGAAAAASNDNA | −0.79 | 0.38 | 0.97 |
| 2616 | ATLVT012XX606 | 45540 | NSAAGAATNDNT | −0.64 | 0.5 | 0.56 |
| 2617 | ATLVT012XX1443 | 46377 | SGQAAAATNDNT | −1.1 | −1.42 | 3.82 |
| 2618 | ATLVT012XX704 | 45638 | NSATGASSNDNH | −0.41 | 1.48 | −1.1 |
| 2619 | ATLVT012XX1345 | 46279 | SGESAAATNDNA | 2.05 | −1.39 | −1.63 |
| 2620 | ATLVT012XX1638 | 46572 | SGTSAASSNDNT | −1.14 | −0.51 | 2.72 |
| 2621 | ATLVT012XX2068 | 47002 | SSQSAASSNDNA | −0.39 | −0.46 | 1.35 |
| 2622 | ATLVT012XX2000 | 46934 | SSETGASTNDNH | −0.14 | 0.73 | −0.61 |
| 2623 | ATLVT012XX3358 | 48292 | TSTSAAASNDNA | −0.92 | 1.25 | 0.07 |
| 2624 | ATLVT012XX1681 | 46615 | SGTTAAATNDNA | −0.15 | −0.1 | 0.47 |
| 2625 | ATLVT012XX3280 | 48214 | TSQTAGSSNDNA | 0 | −0.38 | 0.56 |
| 2626 | ATLVT012XX3234 | 48168 | TSQSAGSSNDNT | −0.81 | 1.24 | −0.14 |
| 2627 | ATLVT012XX2933 | 47867 | TSASAASSNDNH | −0.2 | 0.5 | −0.24 |
| 2628 | ATLVT012XX1889 | 46823 | SSEAAGSSNDNH | −0.11 | 0.46 | −0.34 |
| 2629 | ATLVT012XX1377 | 46311 | SGESGASTNDNT | −0.52 | 0.79 | −0.08 |
| 2630 | ATLVT012XX294 | 45228 | NGQAAAATNDNT | 0.85 | −2.14 | 1.35 |
| 2631 | ATLVT012XX2634 | 47568 | TGQAGGSSNDNT | −0.73 | −0.47 | 1.91 |
| 2632 | ATLVT012XX2553 | 47487 | TGETAASTNDNI | −0.78 | −1.1 | 2.8 |
| 2633 | ATLVT012XX1410 | 46344 | SGETAGSSNDNT | −1.05 | 1.58 | −0.18 |
| 2634 | ATLVT012XX1792 | 46726 | SSASAGSSNDNA | 1.1 | −0.61 | −1.06 |
| 2635 | ATLVT012XX2929 | 47863 | TSASAAATNDNA | 0.93 | −1.06 | −0.18 |
| 2636 | ATLVT012XX897 | 45831 | NSQAGASSNDNT | −0.13 | −0.39 | 0.77 |
| 2637 | ATLVT012XX2433 | 47367 | TGATGASTNDNT | −0.4 | 0.28 | 0.35 |
| 2638 | ATLVT012XX2401 | 47335 | TGATAAATNDNA | 0.05 | −0.24 | 0.25 |
| 2639 | ATLVT012XX2473 | 47407 | TGEAGAATNDNA | −0.85 | 0.87 | 0.35 |
| 2640 | ATLVT012XX1857 | 46791 | SSATGASTNDNT | 1.67 | 1.26 | −1.23 |
| 2641 | ATLVT012XX2482 | 47416 | TGEAGGASNDNA | 0 | 0.06 | −0.07 |
| 2642 | ATLVT012XX3286 | 48220 | TSQTGAASNDNA | −0.4 | −0.16 | 0.89 |
| 2643 | ATLVT012XX2764 | 47698 | TGTAGASSNDNA | 0.06 | −0.1 | 0.02 |
| 2644 | ATLVT012XX1440 | 46374 | SGQAAAASNDNT | −0.7 | −0.77 | 2.18 |
| 2645 | ATLVT012XX1968 | 46902 | SSETAAASNDNT | −0.46 | −0.78 | 1.78 |
| 2646 | ATLVT012XX1560 | 46494 | SGQTGAASNDNT | 0.23 | −0.62 | 0.4 |
| 2647 | ATLVT012XX2112 | 47046 | SSQTAAASNDNT | −0.89 | −0.67 | 2.37 |
| 2648 | ATLVT012XX556 | 45490 | NGTTGAATNDNA | −0.18 | −0.37 | 0.74 |
| 2649 | ATLVT012XX2523 | 47457 | TGESGAATNDNT | −1.02 | −0.21 | 1.99 |
| 2650 | ATLVT012XX796 | 45730 | NSESGAATNDNA | −0.17 | −0.55 | 0.95 |
| 2651 | ATLVT012XX63 | 44997 | NGASAGASNDNI | 0.24 | −0.46 | 0.15 |
| 2652 | ATLVT012XX2674 | 47608 | TGQSGGASNDNA | −0.69 | 1.18 | 0.37 |
| 2653 | ATLVT012XX3293 | 48227 | TSQTGASSNDNH | −0.76 | 0.84 | 0.18 |
| 2654 | ATLVT012XX2645 | 47579 | TGQSAASSNDNH | −0.97 | 0.56 | 0.89 |
| 2655 | ATLVT012XX115 | 45049 | NGATAGSSNDNA | 1.09 | −1.02 | −0.61 |
| 2656 | ATLVT012XX2715 | 47649 | TGQTGAATNDNT | −0.5 | −0.65 | 1.64 |
| 2657 | ATLVT012XX2673 | 47607 | TGQSGASTNDNT | −0.9 | 0.54 | 0.78 |
| 2658 | ATLVT012XX1155 | 46089 | SGAAAAAINDNT | −1.09 | 0.15 | 1.62 |
| 2659 | ATLVT012XX3049 | 47983 | TSEAGAATNDNA | −0.7 | 0.01 | 1.13 |
| 2660 | ATLVT012XX413 | 45347 | NGQTGAATNDNH | −1.03 | 0.24 | 1.4 |
| 2661 | ATLVT012XX870 | 45804 | NSQAAAATNDNT | 0.27 | −0.24 | −0.22 |
| 2662 | ATLVT012XX3139 | 48073 | TSETAGSTNDNA | 1 | −0.99 | −0.53 |
| 2663 | ATLVT012XX370 | 45304 | NGQSGASINDNA | −1.45 | −1.09 | 3.8 |
| 2664 | ATLVT012XX3177 | 48111 | TSQAAASINDNT | −0.23 | −0.76 | 1.29 |
| 2665 | ATLVT012XX3162 | 48096 | TSETGGSSNDNT | 0.23 | −0.25 | −0.16 |
| 2666 | ATLVT012XX1730 | 46664 | SSAAAAAINDNH | 0.95 | −0.85 | −0.62 |
| 2667 | ATLVT012XX2528 | 47462 | TGESGASTNDNH | −0.9 | 0.71 | 0.53 |
| 2668 | ATLVT012XX1776 | 46710 | SSASAAASNDNT | 0.44 | −0.69 | 0.04 |
| 2669 | ATLVT012XX2932 | 47866 | TSASAASSNDNA | 0.46 | −0.45 | −0.31 |
| 2670 | ATLVT012XX3321 | 48255 | TSTAAASTNDNT | −0.65 | 0.18 | 0.79 |
| 2671 | ATLVT012XX1305 | 46239 | SGEAAASTNDNT | −0.02 | −0.06 | 0 |
| 2672 | ATLVT012XX2930 | 47864 | TSASAAATNDNH | 0.24 | −0.72 | 0.39 |
| 2673 | ATLVT012XX1558 | 46492 | SGQTGAASNDNA | −0.43 | −1.02 | 1.92 |
| 2674 | ATLVT012XX3128 | 48062 | TSETAASINDNH | 0.13 | −0.6 | 0.43 |
| 2675 | ATLVT012XX190 | 45124 | NGEAGGSINDNA | −0.18 | 0.98 | −1.08 |
| 2676 | ATLVT012XX2418 | 47352 | TGATAGSSNDNT | 0.01 | −0.12 | 0 |
| 2677 | ATLVT012XX744 | 45678 | NSEAAGSINDNT | −1.46 | 1.96 | −0.15 |
| 2678 | ATLVT012XX2765 | 47699 | TGTAGASSNDNH | −0.41 | −0.08 | 0.67 |
| 2679 | ATLVT012XX3113 | 48047 | TSESGGSSNDNH | −0.32 | 0.16 | 0.2 |
| 2680 | ATLVT012XX3094 | 48028 | TSESGAASNDNA | −0.99 | 0.06 | 1.47 |
| 2681 | ATLVT012XX199 | 45133 | NGESAASSNDNA | −2.01 | −1.57 | 5.32 |
| 2682 | ATLVT012XX3270 | 48204 | TSQTAASSNDNT | −0.37 | −1.06 | 1.86 |
| 2683 | ATLVT012XX131 | 45065 | NGATGASTNDNH | 0.47 | −0.23 | −0.67 |
| 2684 | ATLVT012XX20 | 44954 | NGAAAGSSNDNH | −0.55 | 0.02 | 0.75 |
| 2685 | ATLVT012XX2717 | 47651 | TGQTGASSNDNH | −0.44 | 0.02 | 0.55 |
| 2686 | ATLVT012XX3423 | 48357 | TSTTAGAINDNT | −0.63 | 0.62 | 0.11 |
| 2687 | ATLVT012XX1351 | 46285 | SGESAASINDNA | −0.57 | 0.11 | 0.66 |
| 2688 | ATLVT012XX3191 | 48125 | TSQAGAASNDNH | −0.59 | 0.05 | 0.77 |
| 2689 | ATLVT012XX3313 | 48247 | TSTAAAATNDNA | −0.87 | 0.81 | 0.26 |
| 2690 | ATLVT012XX3267 | 48201 | TSQTAAATNDNT | 0.21 | −0.89 | 0.59 |
| 2691 | ATLVT012XX3070 | 48004 | TSESAAASNDNA | −1.42 | 1.71 | 0.02 |
| 2692 | ATLVT012XX1355 | 46289 | SGESAGASNDNH | −0.74 | −0.08 | 1.17 |
| 2693 | ATLVT012XX257 | 45191 | NGETAGATNDNH | −0.06 | 0.54 | −0.8 |
| 2694 | ATLVT012XX2544 | 47478 | TGETAAASNDNT | 2.29 | −1.94 | −1.63 |
| 2695 | ATLVT012XX343 | 45277 | NGQSAASSNDNA | −0.43 | −0.02 | 0.54 |
| 2696 | ATLVT012XX356 | 45290 | NGQSAGSSNDNH | −0.71 | 0.62 | 0.21 |
| 2697 | ATLVT012XX2600 | 47534 | TGQAAASINDNH | −0.53 | 0.15 | 0.49 |
| 2698 | ATLVT012XX752 | 45686 | NSEAGASSNDNH | −0.61 | 0.49 | 0.17 |
| 2699 | ATLVT012XX1373 | 46307 | SGESGASSNDNH | −0.89 | 1.23 | −0.31 |
| 2700 | ATLVT012XX1834 | 46768 | SSATAGASNDNA | −0.41 | 0.89 | −0.71 |
| 2701 | ATLVT012XX3048 | 47982 | TSEAGAASNDNT | −0.33 | −0.08 | 0.41 |
| 2702 | ATLVT012XX1162 | 46096 | SGAAAGASNDNA | −0.76 | 0.08 | 0.93 |
| 2703 | ATLVT012XX182 | 45116 | NGEAGGASNDNH | 0.31 | −0.56 | −0.08 |
| 2704 | ATLVT012XX121 | 45055 | NGATGAASNDNA | 0.08 | 0.04 | −0.46 |
| 2705 | ATLVT012XX3120 | 48054 | TSETAAASNDNT | 0.48 | −0.86 | 0 |
| 2706 | ATLVT012XX735 | 45669 | NSEAAGASNDNT | −0.64 | 0.39 | 0.3 |
| 2707 | ATLVT012XX871 | 45805 | NSQAAASSNDNA | −0.72 | 1.08 | −0.44 |
| 2708 | ATLVT012XX2417 | 47351 | TGATAGSSNDNH | 0 | 0.05 | −0.36 |
| 2709 | ATLVT012XX1985 | 46919 | SSETAGSSNDNH | −0.52 | 1.22 | −0.98 |
| 2710 | ATLVT012XX1972 | 46906 | SSETAASSNDNA | −1.64 | −1.32 | 4.2 |
| 2711 | ATLVT012XX3025 | 47959 | TSEAAAATNDNA | −0.21 | −0.05 | 0.12 |
| 2712 | ATLVT012XX3223 | 48157 | TSQSAASTNDNA | −0.38 | 0.57 | −0.4 |
| 2713 | ATLVT012XX2124 | 47058 | SSQTAGASNDNT | −1.01 | −0.2 | 1.67 |
| 2714 | ATLVT012XX184 | 45118 | NGEAGGATNDNA | 1.22 | −1.01 | −1.11 |
| 2715 | ATLVT012XX3040 | 47974 | TSEAAGSSNDNA | −0.86 | 0.49 | 0.51 |
| 2716 | ATLVT012XX2475 | 47409 | TGEAGAATNDNT | −0.21 | 0.01 | 0.02 |
| 2717 | ATLVT012XX2994 | 47928 | TSATAGSSNDNT | −0.72 | 0.29 | 0.53 |
| 2718 | ATLVT012XX1279 | 46213 | SGATGASTNDNA | 0.57 | 0.14 | −1.14 |
| 2719 | ATLVT012XX3319 | 48253 | TSTAAASTNDNA | −1.15 | 0.57 | 0.88 |
| 2720 | ATLVT012XX3046 | 47980 | TSEAGAASNDNA | −1.24 | −0.16 | 1.97 |
| 2721 | ATLVT012XX1252 | 46186 | SGATAASSNDNA | 0.31 | −0.18 | −0.66 |
| 2722 | ATLVT012XX1892 | 46826 | SSEAAGSINDNH | 0.09 | 0.19 | −0.76 |
| 2723 | ATLVT012XX1888 | 46822 | SSEAAGSSNDNA | 0.9 | −0.68 | −1.03 |
| 2724 | ATLVT012XX3264 | 48198 | TSQTAAASNDNT | 0.13 | −1.22 | 0.98 |
| 2725 | ATLVT012XX207 | 45141 | NGESAGASNDNT | −1.08 | −0.58 | 2.23 |
| 2726 | ATLVT012XX2699 | 47633 | TGQTAGASNDNH | −0.77 | 0.08 | 0.84 |
| 2727 | ATLVT012XX2077 | 47011 | SSQSAGATNDNA | −0.45 | 0.08 | 0.27 |
| 2728 | ATLVT012XX364 | 45298 | NGQSGAATNDNA | 0.29 | −0.96 | 0.36 |
| 2729 | ATLVT012XX1879 | 46813 | SSEAAASINDNA | 0.66 | −0.88 | −0.39 |
| 2730 | ATLVT012XX242 | 45176 | NGETAAASNDNH | −0.06 | −0.43 | 0.26 |
| 2731 | ATLVT012XX2449 | 47383 | TGEAAAATNDNA | −1.97 | 3.62 | −1.67 |
| 2732 | ATLVT012XX2511 | 47445 | TGESAGAINDNT | −1.75 | 0.4 | 2.09 |
| 2733 | ATLVT012XX160 | 45094 | NGEAAGATNDNA | −1.03 | −0.78 | 2.36 |
| 2734 | ATLVT012XX1832 | 46766 | SSATAASINDNH | 0.37 | −0.29 | −0.66 |
| 2735 | ATLVT012XX2496 | 47430 | TGESAAASNDNT | −1.44 | 1.05 | 0.71 |
| 2736 | ATLVT012XX2705 | 47639 | IGQTAGSSNDNH | −0.69 | 0.76 | −0.21 |
| 2737 | ATLVT012XX972 | 45906 | NSQTAASINDNT | 0.18 | −1.18 | 0.79 |
| 2738 | ATLVT012XX1559 | 46493 | SGQTGAASNDNH | −0.14 | −0.68 | 0.71 |
| 2739 | ATLVT012XX163 | 45097 | NGEAAGSSNDNA | −1.48 | 1.43 | 0.28 |
| 2740 | ATLVT012XX259 | 45193 | NGETAGSSNDNA | −1.81 | 3.14 | −1.36 |
| 2741 | ATLVT012XX1747 | 46681 | SSAAAGSINDNA | −0.93 | 0.56 | 0.46 |
| 2742 | ATLVT012XX8 | 44942 | NGAAAASSNDNH | −1.14 | 0.6 | 0.74 |
| 2743 | ATLVT012XX581 | 45515 | NSAAAAATNDNH | 1.04 | −1.31 | −0.55 |
| 2744 | ATLVT012XX1829 | 46763 | SSATAASSNDNH | −0.85 | 0.18 | 0.78 |
| 2745 | ATLVT012XX1996 | 46930 | SSETGASSNDNA | 1.51 | −2.07 | −0.39 |
| 2746 | ATLVT012XX3235 | 48169 | TSQSAGSTNDNA | −0.96 | 0.79 | 0.17 |
| 2747 | ATLVT012XX597 | 45531 | NSAAAGSSNDNT | −1.33 | 1.06 | 0.46 |
| 2748 | ATLVT012XX686 | 45620 | NSATAGASNDNH | −0.67 | −0.92 | 1.85 |
| 2749 | ATLVT012XX1156 | 46090 | SGAAAASSNDNA | −0.81 | 0.48 | 0.29 |
| 2750 | ATLVT012XX2459 | 47393 | TGEAAGASNDNH | −0.53 | −0.03 | 0.49 |
| 2751 | ATLVT012XX12 | 44946 | NGAAAASTNDNT | −0.74 | 0.09 | 0.69 |
| 2752 | ATLVT012XX3073 | 48007 | TSESAAATNDNA | −1.06 | 0.82 | 0.29 |
| 2753 | ATLVT012XX1640 | 46574 | SGTSAASINDNH | −0.53 | 0.85 | −0.67 |
| 2754 | ATLVT012XX288 | 45222 | NGETGGSTNDNT | −1.36 | 0.73 | 0.91 |
| 2755 | ATLVT012XX2271 | 47205 | SSTTAGATNDNT | −0.82 | 0.72 | 0 |
| 2756 | ATLVT012XX2464 | 47398 | TGEAAGSSNDNA | −0.59 | 0.47 | −0.09 |
| 2757 | ATLVT012XX990 | 45924 | NSQTGAATNDNT | −0.2 | −0.73 | 0.78 |
| 2758 | ATLVT012XX393 | 45327 | NGQTAASSNDNT | −0.5 | −0.57 | 1.09 |
| 2759 | ATLVT012XX2549 | 47483 | TGETAASSNDNH | 0.27 | −0.46 | −0.37 |
| 2760 | ATLVT012XX974 | 45908 | NSQTAGASNDNH | −0.75 | −0.03 | 0.81 |
| 2761 | ATLVT012XX162 | 45096 | NGEAAGAINDNT | −0.54 | −1.11 | 1.81 |
| 2762 | ATLVT012XX1884 | 46818 | SSEAAGASNDNT | −0.95 | 0.21 | 0.81 |
| 2763 | ATLVT012XX2757 | 47691 | TGTAAGSINDNT | −0.58 | 0.09 | 0.33 |
| 2764 | ATLVT012XX738 | 45672 | NSEAAGATNDNT | −0.63 | 0.19 | 0.29 |
| 2765 | ATLVT012XX166 | 45100 | NGEAAGSINDNA | −0.9 | −0.39 | 1.5 |
| 2766 | ATLVT012XX2463 | 47397 | TGEAAGATNDNT | −0.99 | 0.29 | 0.77 |
| 2767 | ATLVT012XX2521 | 47455 | TGESGAATNDNA | −1.05 | 0.4 | 0.73 |
| 2768 | ATLVT012XX3009 | 47943 | TSATGASTNQNT | 0 | −0.44 | 0 |
| 2769 | ATLVT012XX1372 | 46306 | SGESGASSNDNA | −0.74 | 0.01 | 0.69 |
| 2770 | ATLVT012XX342 | 45276 | NGQSAAATNDNT | −0.71 | 1.32 | −1.04 |
| 2771 | ATLVT012XX24 | 44958 | NGAAAGSINDNT | −1.2 | 0.19 | 1.26 |
| 2772 | ATLVT012XX594 | 45528 | NSAAAGATNDNT | −0.83 | 0.23 | 0.55 |
| 2773 | ATLVT012XX2695 | 47629 | TGQTAASTNDNA | −1.23 | 0.02 | 1.5 |
| 2774 | ATLVT012XX1299 | 46233 | SGEAAAATNDNT | 0.04 | −0.59 | 0.1 |
| 2775 | ATLVT012XX3153 | 48087 | TSETGASTNDNT | 0.25 | −0.63 | −0.2 |
| 2776 | ATLVT012XX2497 | 47431 | TGESAAATNDNA | −2.07 | 3.16 | −1.1 |
| 2777 | ATLVT012XX3232 | 48166 | TSQSAGSSNDNA | −0.22 | −0.38 | 0.26 |
| 2778 | ATLVT012XX2551 | 47485 | TGETAASTNDNA | 0.58 | −1 | −0.3 |
| 2779 | ATLVT012XX2493 | 47427 | TGEAGGSINDNT | 0.61 | −0.63 | −0.85 |
| 2780 | ATLVT012XX250 | 45184 | NGETAASTNDNA | 0.56 | −0.36 | −1.12 |
| 2781 | ATLVT012XX2065 | 46999 | SSQSAAAINDNA | −0.46 | −0.84 | 1.25 |
| 2782 | ATLVT012XX3317 | 48251 | TSTAAASSNDNH | −1.07 | 1.15 | −0.31 |
| 2783 | ATLVT012XX652 | 45586 | NSASGAATNDNA | −0.29 | −0.22 | 0.12 |
| 2784 | ATLVT012XX923 | 45857 | NSQSAASINDNH | −0.25 | 0.31 | −0.65 |
| 2785 | ATLVT012XX1842 | 46776 | SSATAGSSNDNT | −0.02 | 0.11 | −0.79 |
| 2786 | ATLVT012XX600 | 45534 | NSAAAGSINDNT | −1.32 | 0.62 | 0.79 |
| 2787 | ATLVT012XX694 | 45628 | NSATAGSTNDNA | −1.56 | −1.08 | 3.37 |
| 2788 | ATLVT012XX2788 | 47722 | IGTSAASSNDNA | −0.19 | 0.11 | −0.5 |
| 2789 | ATLVT012XX1540 | 46474 | SGQTAASSNDNA | 0.34 | −1.26 | 0.36 |
| 2790 | ATLVT012XX360 | 45294 | NGQSAGSINDNT | −0.91 | 0.59 | 0.11 |
| 2791 | ATLVT012XX2208 | 47142 | SSTSAAASNDNT | −0.55 | −1 | 1.55 |
| 2792 | ATLVT012XX792 | 45726 | NSESAGSTNDNT | −0.5 | 0.78 | −0.84 |
| 2793 | ATLVT012XX3172 | 48106 | TSQAAASSNDNA | −0.28 | −0.44 | 0.34 |
| 2794 | ATLVT012XX801 | 45735 | NSESGASSNDNT | 0.01 | −1.13 | 0.72 |
| 2795 | ATLVT012XX56 | 44990 | NGASAASSNDNH | −0.29 | −0.7 | 0.69 |
| 2796 | ATLVT012XX3076 | 48010 | TSESAASSNDNA | −1.15 | 0.53 | 0.56 |
| 2797 | ATLVT012XX109 | 45043 | NGATAGASNDNA | 0.35 | −0.08 | −1.24 |
| 2798 | ATLVT012XX1154 | 46088 | SGAAAAATNDNH | −0.76 | 0.14 | 0.38 |
| 2799 | ATLVT012XX169 | 45103 | NGEAGAASNDNA | −0.47 | −0.37 | 0.53 |
| 2800 | ATLVT012XX2621 | 47555 | TGQAGASSNDNH | −0.98 | 0.31 | 0.52 |
| 2801 | ATLVT012XX400 | 45334 | NGQTAGATNDNA | −0.3 | 0.41 | −0.78 |
| 2802 | ATLVT012XX403 | 45337 | NGQTAGSSNDNA | −0.77 | 0.56 | −0.17 |
| 2803 | ATLVT012XX1941 | 46875 | SSESAGSTNDNT | −1.25 | 0.61 | 0.59 |
| 2804 | ATLVT012XX1353 | 46287 | SGESAASTNDNT | −1.34 | −1.59 | 3.57 |
| 2805 | ATLVT012XX3134 | 48068 | TSETAGATNDNH | −0.42 | 0.06 | −0.14 |
| 2806 | ATLVT012XX2532 | 47466 | TGESGGASNDNT | −1.98 | 0.71 | 1.7 |
| 2807 | ATLVT012XX3228 | 48162 | TSQSAGASNDNT | −0.65 | 0.16 | 0.13 |
| 2808 | ATLVT012XX3201 | 48135 | TSQAGASTNDNT | −1.13 | −0.25 | 1.47 |
| 2809 | ATLVT012XX2543 | 47477 | TGETAAASNDNH | 0.32 | −0.62 | −0.54 |
| 2810 | ATLVT012XX1318 | 46252 | SGEAGAASNDNA | −1.42 | −0.34 | 2.07 |
| 2811 | ATLVT012XX2743 | 47677 | TGTAAASINDNA | −2.78 | −0.66 | 4.81 |
| 2812 | ATLVT012XX2514 | 47448 | TGESAGSSNDNT | −1.53 | 0.54 | 1.12 |
| 2813 | ATLVT012XX3227 | 48161 | TSQSAGASNDNH | −0.83 | 0.26 | 0.28 |
| 2814 | ATLVT012XX783 | 45717 | NSESAGASNDNT | −0.59 | 0.1 | 0.06 |
| 2815 | ATLVT012XX3147 | 48081 | TSETGAATNDNT | −0.25 | −0.5 | 0.25 |
| 2816 | ATLVT012XX3004 | 47938 | TSATGASSNDNA | −1.04 | −1.34 | 2.67 |
| 2817 | ATLVT012XX3218 | 48152 | TSQSAAATNDNH | −0.17 | 0.02 | −0.57 |
| 2818 | ATLVT012XX1948 | 46882 | SSESGASSNDNA | 0.06 | −1.03 | 0.38 |
| 2819 | ATLVT012XX3081 | 48015 | TSESAASTNDNT | −0.32 | −0.03 | −0.26 |
| 2820 | ATLVT012XX263 | 45197 | NGETAGSINDNH | −0.35 | 1.05 | −1.59 |
| 2821 | ATLVT012XX330 | 45264 | NGQAGGATNDNT | −0.05 | −0.81 | 0.26 |
| 2822 | ATLVT012XX1848 | 46782 | SSATGAASNDNT | 0.27 | −0.61 | −0.55 |
| 2823 | ATLVT012XX2522 | 47456 | TGESGAATNDNH | −1.72 | 1.59 | 0.03 |
| 2824 | ATLVT012XX1270 | 46204 | SGATGAASNDNA | −0.63 | 0.44 | −0.36 |
| 2825 | ATLVT012XX3221 | 48155 | TSQSAASSNDNH | −1.34 | 0.39 | 0.91 |
| 2826 | ATLVT012XX1319 | 46253 | SGEAGAASNDNH | −0.22 | −0.66 | 0.33 |
| 2827 | ATLVT012XX3042 | 47976 | TSEAAGSSNDNI | −1.18 | 0.13 | 0.97 |
| 2828 | ATLVT012XX1278 | 46212 | SGATGASSNDNT | −1.05 | 1.22 | −0.66 |
| 2829 | ATLVT012XX2698 | 47632 | TGQTAGASNDNA | −0.61 | −0.18 | 0.38 |
| 2830 | ATLVT012XX108 | 45042 | NGATAASTNDNT | 0.02 | −0.37 | −0.47 |
| 2831 | ATLVT012XX2307 | 47241 | TGAAAAATNDNT | −1.33 | 0.37 | 0.9 |
| 2832 | ATLVT012XX2880 | 47814 | TSAAAAASNDNT | −0.05 | −0.62 | −0.03 |
| 2833 | ATLVT012XX297 | 45231 | NGQAAASSNQNT | 0.76 | −1.73 | 0.01 |
| 2834 | ATLVT012XX2576 | 47510 | TGETGASTNDNH | −0.14 | −1.04 | 0.67 |
| 2835 | ATLVT012XX73 | 45007 | NGASGAASNDNA | −0.18 | −0.6 | 0.17 |
| 2836 | ATLVT012XX2016 | 46950 | SSQAAAASNDNT | −0.32 | −1.54 | 1.61 |
| 2837 | ATLVT012XX1637 | 46571 | SGTSAASSNDNH | −1.03 | −0.36 | 1.3 |
| 2838 | ATLVT012XX3246 | 48180 | TSQSGASSNDNT | −1.52 | 0.6 | 0.91 |
| 2839 | ATLVT012XX1584 | 46518 | SGTAAAASNDNT | −0.78 | −0.93 | 1.62 |
| 2840 | ATLVT012XX2782 | 47716 | TGTSAAASNDNA | 0.04 | −1.54 | 1 |
| 2841 | ATLVT012XX3411 | 48345 | TSTTAAATNDNT | −0.12 | −0.43 | −0.16 |
| 2842 | ATLVT012XX992 | 45926 | NSQTGASSNDNH | −0.55 | 0.09 | −0.1 |
| 2843 | ATLVT012XX788 | 45722 | NSESAGSSNDNH | −0.22 | 0.39 | −1.06 |
| 2844 | ATLVT012XX3005 | 47939 | TSATGASSNDNH | −0.38 | 0.28 | −0.64 |
| 2845 | ATLVT012XX2704 | 47638 | TGQTAGSSNDNA | −0.83 | 0.77 | −0.51 |
| 2846 | ATLVT012XX2211 | 47145 | SSTSAAATNDNT | −0.92 | −0.53 | 1.31 |
| 2847 | ATLVT012XX2487 | 47421 | TGEAGGAINDNT | −0.53 | 0.09 | −0.16 |
| 2848 | ATLVT012XX1419 | 46353 | SGETGAATNDNT | −0.51 | −1.57 | 1.94 |
| 2849 | ATLVT012XX3364 | 48298 | TSTSAASSNDNA | −1.96 | −0.09 | 2.51 |
| 2850 | ATLVT01XXX3214 | 48148 | TSQSAAASNDNA | −0.97 | −0.04 | 0.75 |
| 2851 | ATLVT012XX2979 | 47913 | TSATAAATNDNT | −1.22 | 0.83 | 0.05 |
| 2852 | ATLVT012XX1321 | 46255 | SGEAGAATNDNA | −0.51 | −0.89 | 1.03 |
| 2853 | ATLVT012XX1942 | 46876 | SSESGAASNDNA | −0.68 | −0.33 | 0.62 |
| 2854 | ATLVT012XX2712 | 47646 | TGQTGAASNDNT | −0.56 | −0.35 | 0.44 |
| 2855 | ATLVT012XX354 | 45288 | NGQSAGAINDNT | −1.18 | −0.02 | 1.06 |
| 2856 | ATLVT012XX2412 | 47346 | TGATAGASNDNT | −0.78 | −0.24 | 0.65 |
| 2857 | ATLVT012XX2577 | 47511 | TGETGASINDNT | −0.92 | 1.8 | 2.9 |
| 2858 | ATLVT012XX3140 | 48074 | TSETAGSINDNH | −0.61 | −0.04 | 0.11 |
| 2859 | ATLVT012XX325 | 45259 | NGQAGGASNDNA | −0.9 | 0.08 | 0.45 |
| 2860 | ATLVT012XX1998 | 46932 | SSETGASSNDNT | 0.62 | −1.38 | −0.29 |
| 2861 | ATLVT012XX205 | 45139 | NGESAGASNDNA | −1.95 | −0.69 | 3.23 |
| 2862 | ATLVT012XX13 | 44947 | NGAAAGASNDNA | −1.23 | 0.16 | 0.9 |
| 2863 | ATLVT012XX2024 | 46958 | SSQAAASINDNH | −0.62 | −0.22 | 0.32 |
| 2864 | ATLVT012XX114 | 45048 | NGATAGAINDNT | −0.68 | 0.05 | 0.09 |
| 2865 | ATLVT012XX2350 | 47284 | TGASAAASNDNA | −0.74 | 0.4 | −0.26 |
| 2866 | ATLVT012XX746 | 45680 | NSEAGAASNDNH | −0.89 | −0.25 | 0.82 |
| 2867 | ATLVT012XX3045 | 47979 | TSEAAGSINDNT | −1.26 | 0.24 | 0.83 |
| 2868 | ATLVT012XX1978 | 46912 | SSETAGASNDNA | −0.6 | −0.12 | 0.14 |
| 2869 | ATLVT012XX2566 | 47500 | TGETGAASNDNA | −0.61 | −0.32 | 0.4 |
| 2870 | ATLVT012XX800 | 45734 | NSESGASSNDNH | −0.27 | −0.33 | −0.17 |
| 2871 | ATLVT012XX46 | 44980 | NGAAGGSINDNA | −1.23 | −0.17 | 1.27 |
| 2872 | ATLVT012XX778 | 45712 | NSESAASINDNA | −0.8 | 1.06 | −1.05 |
| 2873 | ATLVT012XX2114 | 47048 | SSQTAAAINDNH | −0.53 | −0.3 | 0.23 |
| 2874 | ATLVT012XX149 | 45083 | NGEAAAATNDNH | −0.17 | 0.75 | −1.74 |
| 2875 | ATLVT012XX755 | 45689 | NSEAGASTNDNH | −0.47 | −0.37 | 0.21 |
| 2876 | ATLVT012XX2555 | 47489 | TGETAGASNDNH | −1.18 | 0.5 | 0.31 |
| 2877 | ATLVT012XX2563 | 47497 | TGETAGSINDNA | −0.63 | 0.84 | −1.08 |
| 2878 | ATLVT012XX2854 | 47788 | TGTTGAASNDNA | −0.93 | −0.28 | 0.87 |
| 2879 | ATLVT012XX1687 | 46621 | SGTTAASTNDNA | −1.06 | −0.84 | 1.79 |
| 2880 | ATLVT012XX3136 | 48070 | TSETAGSSNDNA | −0.43 | −0.49 | 0.25 |
| 2881 | ATLVT012XX1264 | 46198 | SGATAGSSNDNA | 0.65 | −0.46 | −1.66 |
| 2882 | ATLVT012XX2745 | 47679 | TGTAAASTNDNT | −0.55 | −1.13 | 1.27 |
| 2883 | ATLVT012XX1396 | 46330 | SGETAASSNDNA | 0.01 | −0.43 | −0.6 |
| 2884 | ATLVT012XX3408 | 48342 | TSTTAAASNDNT | −0.54 | −0.82 | 0.84 |
| 2885 | ATLVT012XX3047 | 47981 | TSEAGAASNDNH | −0.79 | −0.37 | 0.69 |
| 2886 | ATLVT012XX3029 | 47963 | TSEAAASSNDNH | −0.4 | −0.53 | 0.21 |
| 2887 | ATLVT012XX3082 | 48016 | TSESAGASNDNA | −1.56 | 0.39 | 1.03 |
| 2888 | ATLVT012XX1736 | 46670 | SSAAAASINDNH | −1.47 | 0.34 | 0.92 |
| 2889 | ATLVT012XX2571 | 47505 | TGETGAATNDNT | −0.47 | −0.43 | 0.2 |
| 2890 | ATLVT012XX1931 | 46865 | SSESAGASNDNH | −1.94 | 1.1 | 0.73 |
| 2891 | ATLVT012XX2740 | 47674 | TGTAAASSNDNA | −1.03 | 0.17 | 0.37 |
| 2892 | ATLVT012XX50 | 44984 | NGASAAASNDNH | −0.43 | −0.94 | 0.78 |
| 2893 | ATLVT012XX1856 | 46790 | SSATGASTNDNH | 0.39 | −0.09 | −1.73 |
| 2894 | ATLVT012XX965 | 45899 | NSQTAAATNDNH | −0.27 | −0.72 | 0.21 |
| 2895 | ATLVT012XX1344 | 46278 | SGESAAASNDNT | 0.2 | −1.36 | 0.22 |
| 2896 | ATLVT012XX2403 | 47337 | TGATAAATNDNT | −1.98 | −0.41 | 2.74 |
| 2897 | ATLVT012XX151 | 45085 | NGEAAASSNDNA | −0.63 | 0.88 | −1.24 |
| 2898 | ATLVT012XX3022 | 47956 | TSEAAAASNDNA | 1.15 | −1.59 | −1.12 |
| 2899 | ATLVT012XX200 | 45134 | NGESAASSNDNH | −1.56 | −0.08 | 1.58 |
| 2900 | ATLVT012XX416 | 45350 | NGQTGASSNDNH | −2.1 | −0.29 | 2.76 |
| 2901 | ATLVT012XX1885 | 46819 | SSEAAGAINDNA | −1.13 | 0.45 | 0.12 |
| 2902 | ATLVT012XX2472 | 47406 | TGEAGAASNDNT | −1.05 | 0.53 | −0.11 |
| 2903 | ATLVT012XX1314 | 46248 | SGEAAGSSNDNT | −0.39 | −0.61 | 0.22 |
| 2904 | ATLVT012XX979 | 45913 | NSQTAGSSNDNA | 0.2 | −1.23 | 0 |
| 2905 | ATLVT012XX3099 | 48033 | TSESGAATNDNT | −0.75 | −0.12 | 0.2 |
| 2906 | ATLVT012XX3229 | 48163 | TSQSAGAINDNA | −1.03 | 0.46 | −0.07 |
| 2907 | ATLVT012XX164 | 45098 | NGEAAGSSNDNH | −1.12 | 0.01 | 0.66 |
| 2908 | ATLVT012XX2209 | 47143 | SSTSAAATNDNA | −0.47 | −0.44 | 0.11 |
| 2909 | ATLVT012XX657 | 45591 | NSASGASSNDNT | −0.21 | −0.35 | −0.46 |
| 2910 | ATLVT012XX252 | 45186 | NGETAASTNDNT | −0.27 | 0.07 | −0.89 |
| 2911 | ATLVT012XX3030 | 47964 | TSEAAASSNDNT | −0.43 | −0.55 | 0.17 |
| 2912 | ATLVT012XX964 | 45898 | NSQTAAATNDNA | −0.2 | −1.31 | 0.75 |
| 2913 | ATLVT012XX2620 | 47554 | TGQAGASSNDNA | −1.28 | 0.59 | 0.15 |
| 2914 | ATLVT012XX2648 | 47582 | TGQSAASINDNH | −1.05 | 0.54 | −0.17 |
| 2915 | ATLVT012XX781 | 45715 | NSESAGASNDNA | −0.35 | −0.15 | −0.51 |
| 2916 | ATLVT012XX2169 | 47103 | SSTAAASINDNT | −0.04 | −0.48 | −0.59 |
| 2917 | ATLVT012XX1782 | 46716 | SSASAASSNDNT | −1.05 | −0.26 | 0.85 |
| 2918 | ATLVT012XX2598 | 47532 | TGQAAASSNDNT | −0.79 | −0.66 | 0.88 |
| 2919 | ATLVT012XX1422 | 46356 | SGETGASSNDNT | −1.42 | 0.6 | 0.35 |
| 2920 | ATLVT012XX485 | 45419 | NGTSAAATNDNH | −0.34 | −0.47 | −0.13 |
| 2921 | ATLVT012XX1583 | 46517 | SGTAAAASNDNH | −1.43 | 0.41 | 0.61 |
| 2922 | ATLVT012XX58 | 44992 | NGASAASTNDNA | −0.43 | −0.08 | −0.49 |
| 2923 | ATLVT012XX1846 | 46780 | SSATGAASNDNA | −0.15 | −0.32 | −0.66 |
| 2924 | ATLVT012XX1222 | 46156 | SGASGAASNDNA | −0.04 | −0.8 | −0.24 |
| 2925 | ATLVT012XX3103 | 48037 | TSESGASTNDNA | −2.03 | 1.95 | −0.4 |
| 2926 | ATLVT012XX1794 | 46728 | SSASAGSSNDNT | −0.17 | −0.93 | 0.12 |
| 2927 | ATLVT012XX2689 | 47623 | TGQTAAATNDNA | −0.81 | −0.11 | 0.15 |
| 2928 | ATLVT012XX2996 | 47930 | TSATAGSINDNH | −0.44 | −0.51 | 0.02 |
| 2929 | ATLVT012XX3233 | 48167 | TSQSAGSSNDNH | −0.83 | 0.26 | −0.31 |
| 2930 | ATLVT012XX402 | 45336 | NGQTAGAINDNT | 0.18 | −0.76 | −0.73 |
| 2931 | ATLVT012XX2574 | 47508 | TGETGASSNDNT | −0.71 | −0.98 | 1.07 |
| 2932 | ATLVT012XX2533 | 47467 | TGESGGATNDNA | −1.82 | 0.33 | 1.29 |
| 2933 | ATLVT012XX10 | 44944 | NGAAAASTNDNA | −1.88 | 0.9 | 0.66 |
| 2934 | ATLVT012XX751 | 45685 | NSEAGASSNDNA | −0.93 | −0.04 | 0.24 |
| 2935 | ATLVT012XX290 | 45224 | NGQAAAASNDNH | 0.14 | −1.83 | 0.71 |
| 2936 | ATLVT012XX3151 | 48085 | TSETGASINDNA | −0.59 | 0.02 | −0.44 |
| 2937 | ATLVT012XX2505 | 47439 | TGESAASTNDNT | −1.65 | 0.53 | 0.73 |
| 2938 | ATLVT012XX2302 | 47236 | TGAAAAASNDNA | −1.42 | 0.22 | 0.73 |
| 2939 | ATLVT012XX2451 | 47385 | TGEAAAATNDNT | −0.27 | −0.3 | −0.58 |
| 2940 | ATLVT012XX175 | 45109 | NGEAGASSNDNA | −0.39 | 0.67 | −1.63 |
| 2941 | ATLVT012XX724 | 45658 | NSEAAAATNDNA | −2.26 | 3.14 | −1.6 |
| 2942 | ATLVT012XX3133 | 48067 | TSETAGAINDNA | −1.07 | 0.44 | −0.17 |
| 2943 | ATLVT012XX156 | 45090 | NGEAAASTNDNT | −2.04 | 2.83 | −1.59 |
| 2944 | ATLVT012XX698 | 45632 | NSATGAASNDNH | −0.2 | −0.84 | −0.03 |
| 2945 | ATLVT012XX2170 | 47104 | SSTAAGASNDNA | −1.56 | 0.24 | 0.9 |
| 2946 | ATLVT012XX168 | 45102 | NGEAAGSINDNT | −0.82 | −0.95 | 1.16 |
| 2947 | ATLVT012XX2354 | 47288 | TGASAAATNDNH | −0.88 | 0.21 | −0.24 |
| 2948 | ATLVT012XX2477 | 47411 | TGEAGASSNDNH | −0.73 | 0.26 | −0.56 |
| 2949 | ATLVT012XX417 | 45351 | NGQTGASSNDNT | −1.45 | −0.68 | 1.88 |
| 2950 | ATLVT012XX844 | 45778 | NSETGAATNDNA | −0.79 | −0.42 | 0.42 |
| 2951 | ATLVT012XX1878 | 46812 | SSEAAASSNDNT | −0.38 | −0.72 | 0.08 |
| 2952 | ATLVT012XX1312 | 46246 | SGEAAGSSNDNA | −0.41 | −0.57 | −0.04 |
| 2953 | ATLVT012XX1980 | 46914 | SSETAGASNDNT | −0.67 | −0.09 | −0.22 |
| 2954 | ATLVT012XX2927 | 47861 | TSASAAASNDNH | −0.81 | −0.5 | 0.54 |
| 2955 | ATLVT012XX2658 | 47592 | TGQSAGSSNDNT | −0.84 | 0.63 | −0.86 |
| 2956 | ATLVT012XX3226 | 48160 | TSQSAGASNDNA | −0.98 | −0.26 | 0.51 |
| 2957 | ATLVT012XX59 | 44993 | NGASAASINDNH | −0.86 | 0.5 | −0.68 |
| 2958 | ATLVT012XX67 | 45001 | NGASAGSSNDNA | 0.68 | −1.31 | −0.99 |
| 2959 | ATLVT012XX3196 | 48130 | TSQAGASSNDNA | −1.01 | −0.25 | 0.53 |
| 2960 | ATLVT012XX2255 | 47189 | SSTTAAASNDNH | −1.71 | 1.54 | −0.55 |
| 2961 | ATLVT012XX3097 | 48031 | TSESGAATNDNA | −0.93 | −0.28 | 0.45 |
| 2962 | ATLVT012XX2455 | 47389 | TGEAAASTNDNA | −0.53 | 0.23 | −0.9 |
| 2963 | ATLVT012XX1929 | 46863 | SSESAASINDNT | −0.22 | −0.47 | −0.56 |
| 2964 | ATLVT012XX2641 | 47575 | TGQSAAATNDNA | −1.32 | 0.38 | 0.24 |
| 2965 | ATLVT012XX2995 | 47929 | TSATAGSINDNA | −1.15 | 0.48 | −0.18 |
| 2966 | ATLVT012XX1399 | 46333 | SGETAASTNDNA | −0.09 | −0.76 | −0.41 |
| 2967 | ATLVT012XX2500 | 47434 | TGESAASSNDNA | −1.84 | 1.02 | 0.29 |
| 2968 | ATLVT012XX1732 | 46666 | SSAAAASSNDNA | −0.19 | −0.83 | −0.15 |
| 2969 | ATLVT012XX3219 | 48153 | TSQSAAATNDNT | −1.36 | 0.05 | 0.72 |
| 2970 | ATLVT012XX1988 | 46922 | SSETAGSTNDNH | −0.68 | −0.24 | −0.08 |
| 2971 | ATLVT012XX1893 | 46827 | SSEAAGSINDNT | −0.8 | 0.6 | −0.97 |
| 2972 | ATLVT012XX2262 | 47196 | SSTTAASSNDNT | −1.24 | 0.89 | −0.6 |
| 2973 | ATLVT012XX2591 | 47525 | TGQAAAASNDNH | −1.81 | −0.36 | 1.99 |
| 2974 | ATLVT012XX577 | 45511 | NSAAAAASNDNA | 0.54 | −1.68 | −0.33 |
| 2975 | ATLVT012XX157 | 45091 | NGEAAGASNDNA | −0.9 | −0.83 | 1.03 |
| 2976 | ATLVT012XX922 | 45856 | NSQSAASTNDNA | −0.24 | −0.4 | −0.66 |
| 2977 | ATLVT012XX784 | 45718 | NSESAGATNDNA | −0.67 | 0.27 | −0.77 |
| 2978 | ATLVT012XX2548 | 47482 | TGETAASSNDNA | −1.03 | 0.16 | −0.03 |
| 2979 | ATLVT012XX2258 | 47192 | SSTTAAATNDNH | −0.83 | 1.1 | −1.6 |
| 2980 | ATLVT012XX61 | 44995 | NGASAGASNDNA | −0.66 | −0.15 | −0.28 |
| 2981 | ATLVT012XX1777 | 46711 | SSASAAATNDNA | 0.03 | −0.69 | −0.78 |
| 2982 | ATLVT012XX875 | 45809 | NSQAAASTNDNH | −1.19 | 0.73 | −0.53 |
| 2983 | ATLVT012XX2460 | 47394 | TGEAAGASNDNT | −1.47 | 0.64 | 0.08 |
| 2984 | ATLVT012XX787 | 45721 | NSESAGSSNDNA | −0.74 | −0.76 | 0.61 |
| 2985 | ATLVT012XX1843 | 46777 | SSATAGSINDNA | 0.06 | −0.52 | −1.06 |
| 2986 | ATLVT012XX2983 | 47917 | TSATAASTNDNA | −0.69 | 0.14 | −0.65 |
| 2987 | ATLVT012XX1535 | 46469 | SGQTAAASNDNH | −0.65 | −1.05 | 0.82 |
| 2988 | ATLVT012XX1266 | 46200 | SGATAGSSNDNT | −0.41 | −0.08 | −0.84 |
| 2989 | ATLVT012XX183 | 45117 | NGEAGGASNDNT | 0.26 | −0.92 | −0.93 |
| 2990 | ATLVT012XX1445 | 46379 | SGQAAASSNDNH | −0.6 | −0.59 | 0.14 |
| 2991 | ATLVT012XX2410 | 47344 | TGATAGASNDNA | −1.01 | −0.16 | 0.28 |
| 2992 | ATLVT012XX2425 | 47359 | TGATGAATNDNA | −1.27 | 0.17 | 0.3 |
| 2993 | ATLVT012XX1774 | 46708 | SSASAAASNDNA | 0.24 | −0.88 | −0.94 |
| 2994 | ATLVT012XX2073 | 47007 | SSQSAASINDNT | −0.13 | −1.39 | 0.35 |
| 2995 | ATLVT012XX345 | 45279 | NGQSAASSNDNT | −2.51 | 2.68 | −0.84 |
| 2996 | ATLVT012XX985 | 45919 | NSQTGAASNDNA | −0.86 | −0.36 | 0.24 |
| 2997 | ATLVT012XX2018 | 46952 | SSQAAAATNDNH | −1.47 | −0.78 | 1.83 |
| 2998 | ATLVT012XX2742 | 47676 | TGTAAASSNDNT | −1.36 | −0.5 | 1.27 |
| 2999 | ATLVT012XX740 | 45674 | NSEAAGSSNDNH | −1.27 | 0.5 | −0.18 |
| 3000 | ATLVT012XX2122 | 47056 | SSQTAGASNDNA | −1.37 | −0.22 | 0.92 |
| 3001 | ATLVT012XX1442 | 46376 | SGQAAAATNDNH | −1.65 | −0.31 | 1.52 |
| 3002 | ATLVT012XX1324 | 46258 | SGEAGASSNDNA | 0.91 | −1.23 | −1.71 |
| 3003 | ATLVT012XX1990 | 46924 | SSETGAASNDNA | −0.18 | −1.34 | 0.29 |
| 3004 | ATLVT012XX742 | 45676 | NSEAAGSINDNA | −1.2 | 0.73 | −0.61 |
| 3005 | ATLVT012XX532 | 45466 | NGTTAAATNDNA | −1.11 | −0.14 | 0.36 |
| 3006 | ATLVT012XX2882 | 47816 | TSAAAAATNDNH | −1.24 | 0.46 | −0.2 |
| 3007 | ATLVT012XX821 | 45755 | NSETAAATNDNH | −0.56 | 0.02 | −0.81 |
| 3008 | ATLVT012XX2167 | 47101 | SSTAAASINDNA | −1 | −0.51 | 0.63 |
| 3009 | ATLVT012XX1636 | 46570 | SGTSAASSNDNA | −0.98 | 0 | −0.07 |
| 3010 | ATLVT012XX358 | 45292 | NGQSAGSTNDNA | −1 | 0.02 | −0.07 |
| 3011 | ATLVT012XX254 | 45188 | NGETAGASNDNH | −1.4 | 0.59 | −0.12 |
| 3012 | ATLVT012XX3197 | 48131 | TSQAGASSNDNH | −0.92 | −0.26 | 0.13 |
| 3013 | ATLVT012XX1301 | 46235 | SGEAAASSNDNH | −0.57 | −0.62 | −0.01 |
| 3014 | ATLVT012XX635 | 45569 | NSASAASTNDNH | −1.19 | 0.96 | −0.95 |
| 3015 | ATLVT012XX3169 | 48103 | TSQAAAATNDNA | −1.3 | −0.99 | 1.71 |
| 3016 | ATLVT012XX3224 | 48158 | TSQSAASINDNH | −0.84 | −0.1 | −0.21 |
| 3017 | ATLVT012XX14 | 44948 | NGAAAGASNDNH | −1.06 | −0.23 | 0.33 |
| 3018 | ATLVT012XX3406 | 48340 | TSTTAAASNDNA | −0.39 | −0.58 | −0.39 |
| 3019 | ATLVT012XX1491 | 46425 | SGQSAAATNDNT | −0.12 | −1.07 | −0.22 |
| 3020 | ATLVT012XX152 | 45086 | NGEAAASSNDNH | −0.88 | 0.22 | −0.57 |
| 3021 | ATLVT012XX2502 | 47436 | TGESAASSNDNT | −1.54 | 0.7 | −0.06 |
| 3022 | ATLVT012XX280 | 45214 | NGETGGATNDNA | −1.33 | 0.28 | 0.1 |
| 3023 | ATLVT012XX777 | 45711 | NSESAASSNDNT | −0.39 | −0.16 | −0.96 |
| 3024 | ATLVT012XX2738 | 47672 | TGTAAAATNDNH | −1.48 | −0.18 | 0.93 |
| 3025 | ATLVT012XX3074 | 48008 | TSESAAATNDNH | −1.01 | 0 | −0.09 |
| 3026 | ATLVT012XX2166 | 47100 | SSTAAASSNDNT | −0.74 | −0.42 | −0.02 |
| 3027 | ATLVT012XX994 | 45928 | NSQTGASINDNA | −0.62 | 0.61 | −1.55 |
| 3028 | ATLVT012XX587 | 45521 | NSAAAASINDNH | −2.13 | 0.56 | 1.09 |
| 3029 | ATLVT012XX3043 | 47977 | TSEAAGSINDNA | −1.63 | 0.26 | 0.62 |
| 3030 | ATLVT012XX2670 | 47604 | TGQSGASSNDNT | −1.81 | −0.42 | 1.8 |
| 3031 | ATLVT012XX1414 | 46348 | SGETGAASNDNA | −1.46 | −0.08 | 0.76 |
| 3032 | ATLVT012XX2934 | 47868 | TSASAASSNDNT | −0.58 | −0.87 | 0.26 |
| 3033 | ATLVT012XX102 | 45036 | NGATAAATNDNT | −1.3 | 0.6 | −0.41 |
| 3034 | ATLVT012XX2508 | 47442 | TGESAGASNDNT | −2.28 | 0.1 | 1.91 |
| 3035 | ATLVT012XX1448 | 46382 | SGQAAASTNDNH | 0.64 | −1.28 | −1.34 |
| 3036 | ATLVT012XX296 | 45230 | NGQAAASSNDNH | −0.46 | −0.4 | −0.6 |
| 3037 | ATLVT012XX961 | 45895 | NSQTAAASNDNA | −0.39 | −0.98 | 0.03 |
| 3038 | ATLVT012XX2688 | 47622 | TGQTAAASNDNT | −0.66 | −1.31 | 0.92 |
| 3039 | ATLVT012XX2272 | 47206 | SSTTAGSSNDNA | −0.17 | −0.43 | −1.06 |
| 3040 | ATLVT012XX2643 | 47577 | TGQSAAATNDNT | −1.88 | 0.08 | 1.22 |
| 3041 | ATLVT012XX3026 | 47960 | TSEAAAATNDNH | −0.38 | −0.95 | −0.04 |
| 3042 | ATLVT012XX753 | 45687 | NSEAGASSNDNT | −1.65 | 0.44 | 0.34 |
| 3043 | ATLVT012XX2494 | 47428 | TGESAAASNDNA | -2.28 | 1.33 | 0.28 |
| 3044 | ATLVT012XX227 | 45161 | NGESGASTNDNH | −0.94 | 0.58 | −1.06 |
| 3045 | ATLVT012XX1362 | 46296 | SGESAGSSNDNT | −1.8 | 0.9 | −0.02 |
| 3046 | ATLVT012XX2590 | 47524 | TGQAAAASNDNA | −1.22 | −0.77 | 1.11 |
| 3047 | ATLVT012XX2601 | 47535 | TGQAAASTNDNT | −0.98 | −0.78 | 0.71 |
| 3048 | ATLVT012XX1012 | 45946 | NSTAAAATNDNA | −1.3 | −1.13 | 1.71 |
| 3049 | ATLVT012XX1251 | 46185 | SGATAAATNDNT | 0 | −1.19 | −0.46 |
| 3050 | ATLVT012XX829 | 45763 | NSETAGASNDNA | −1.52 | 0.46 | 0.02 |
| 3051 | ATLVT012XX1837 | 46771 | SSATAGATNDNA | 0.15 | −0.6 | −1.47 |
| 3052 | ATLVT012XX2766 | 47700 | IGTAGASSNDNT | −1.25 | −0.44 | 0.73 |
| 3053 | ATLVT012XX3311 | 48245 | TSTAAAASNDNH | −1.53 | 0.77 | −0.35 |
| 3054 | ATLVT012XX2614 | 47548 | TGQAGAASNDNA | −1.66 | 0.56 | 0.15 |
| 3055 | ATLVT012XX2019 | 46953 | SSQAAAAINDNT | −1.5 | −1.33 | 2.28 |
| 3056 | ATLVT012XX2839 | 47773 | TGTTAASINDNA | -1.94 | 0.62 | 0.52 |
| 3057 | ATLVT012XX2353 | 47287 | TGASAAATNDNA | −1.25 | 0.05 | 0.07 |
| 3058 | ATLVT012XX3080 | 48014 | TSESAASTNDNH | −1.84 | 0.9 | −0.02 |
| 3059 | ATLVT012XX2358 | 47292 | TGASAASSNDNT | −2.42 | 1.19 | 0.59 |
| 3060 | ATLVT012XX220 | 45154 | NGESGAATNDNA | −0.51 | −1.21 | 0.4 |
| 3061 | ATLVT012XX2547 | 47481 | TGETAAATNDNT | 0.34 | −1.35 | −0.88 |
| 3062 | ATLVT012XX2448 | 47382 | TGEAAAASNDNT | −0.48 | −0.83 | −0.14 |
| 3063 | ATLVT012XX195 | 45129 | NGESAAASNDNT | −1.42 | −1.23 | 1.97 |
| 3064 | ATLVT012XX1486 | 46420 | SGQSAAASNDNA | 0.61 | −1.49 | −1.17 |
| 3065 | ATLVT012XX2701 | 47635 | TGQTAGAINDNA | −0.79 | −0.61 | 0.07 |
| 3066 | ATLVT012XX3132 | 48066 | TSETAGASNDNT | −1.44 | 0.3 | 0 |
| 3067 | ATLVT012XX831 | 45765 | NSETAGASNDNT | −0.6 | −0.17 | −0.83 |
| 3068 | ATLVT012XX2879 | 47813 | TSAAAAASNDNH | −1.09 | −0.08 | −0.1 |
| 3069 | ATLVT012XX2467 | 47401 | TGEAAGSTNDNA | −1.09 | 0.1 | −0.34 |
| 3070 | ATLVT012XX2785 | 47719 | TGTSAAATNDNA | −0.77 | −0.47 | −0.19 |
| 3071 | ATLVT012XX993 | 45927 | NSQTGASSNDNT | 0.06 | −0.94 | −1.02 |
| 3072 | ATLVT012XX2476 | 47410 | TGEAGASSNDNA | −0.49 | −0.12 | −1.13 |
| 3073 | ATLVT012XX3412 | 48346 | TSTTAASSNDNA | −1.94 | 0.11 | 1.07 |
| 3074 | ATLVT012XX2176 | 47110 | SSTAAGSSNDNA | −1.15 | −0.64 | 0.66 |
| 3075 | ATLVT012XX153 | 45087 | NGEAAASSNDNT | 0.09 | −0.83 | −1.24 |
| 3076 | ATLVT012XX804 | 45738 | NSESGASTNDNT | 0.92 | −2.12 | −0.99 |
| 3077 | ATLVT012XX3032 | 47966 | TSEAAASINDNH | −1.12 | −0.18 | 0.01 |
| 3078 | ATLVT012XX3072 | 48006 | TSESAAASNDNT | −1.71 | −0.1 | 0.9 |
| 3079 | ATLVT012XX1323 | 46257 | SGEAGAATNDNT | 1.1 | −1.12 | 1.16 |
| 3080 | ATLVT012XX1447 | 46381 | SGQAAASTNDNA | −0.47 | −1.3 | 0.29 |
| 3081 | ATLVT012XX105 | 45039 | NGATAASSNDNT | −1.38 | 0.1 | 0.06 |
| 3082 | ATLVT012XX823 | 45757 | NSETAASSNDNA | −1.35 | 1.18 | −1.39 |
| 3083 | ATLVT012XX2997 | 47931 | TSATAGSINDNT | −1.22 | −0.38 | 0.38 |
| 3084 | ATLVT012XX2206 | 47140 | SSTSAAASNDNA | −0.61 | −0.39 | −0.66 |
| 3085 | ATLVT012XX2744 | 47678 | TGTAAASTNDNH | −1.36 | −0.87 | 1.26 |
| 3086 | ATLVT012XX172 | 45106 | NGEAGAATNDNA | −0.36 | −0.73 | −0.64 |
| 3087 | ATLVT012XX486 | 45420 | NGTSAAATNDNT | −1 | −0.86 | 0.62 |
| 3088 | ATLVT012XX1887 | 46821 | SSEAAGATNDNT | −1.3 | 0.04 | −0.03 |
| 3089 | ATLVT012XX1206 | 46140 | SGASAASSNDNT | −0.85 | −0.7 | 0.14 |
| 3090 | ATLVT012XX2022 | 46956 | SSQAAASSNDNT | −1.71 | −0.25 | 1.03 |
| 3091 | ATLVT012XX1930 | 46864 | SSESAGASNDNA | −1.57 | 0.16 | 0.25 |
| 3092 | ATLVT012XX2159 | 47093 | SSTAAAASNDNH | −2.25 | 1.16 | 0.12 |
| 3093 | ATLVT012XX633 | 45567 | NSASAASSNDNT | −1.58 | −0.11 | 0.59 |
| 3094 | ATLVT012XX1950 | 46884 | SSESGASSNDNT | −1.2 | −0.43 | 0.36 |
| 3095 | ATLVT012XX203 | 45137 | NGESAASINDNH | −1.2 | 0.38 | −0.69 |
| 3096 | ATLVT012XX2623 | 47557 | TGQAGASINDNA | −1.83 | −0.24 | 1.16 |
| 3097 | ATLVT012XX3006 | 47940 | TSATGASSNDNT | −1.05 | −0.27 | −0.15 |
| 3098 | ATLVT012XX357 | 45291 | NGQSAGSSNDNT | −1.62 | 0.4 | 0.03 |
| 3099 | ATLVT012XX2710 | 47644 | TGQTGAASNDNA | −1.42 | −0.53 | 0.82 |
| 3100 | ATLVT012XX313 | 45247 | NGQAGAASNDNA | −0.22 | −1.1 | −0.51 |
| 3101 | ATLVT012XX1785 | 46719 | SSASAASTNDNT | −0.48 | −0.32 | −1.07 |
| 3102 | ATLVT012XX3310 | 48244 | TSTAAAASNDNA | −1.11 | −0.13 | −0.23 |
| 3103 | ATLVT012XX732 | 45666 | NSEAAASTNDNT | −2.86 | 3.02 | −1.29 |
| 3104 | ATLVT012XX991 | 45925 | NSQTGASSNDNA | −0.19 | −0.8 | −0.96 |
| 3105 | ATLVT012XX3028 | 47962 | TSEAAASSNDNA | −0.99 | −0.23 | −0.32 |
| 3106 | ATLVT012XX103 | 45037 | NGATAASSNDNA | −0.39 | −0.14 | −1.48 |
| 3107 | ATLVT012XX3054 | 47988 | TSEAGASSNDNT | −1.3 | −0.68 | 0.77 |
| 3108 | ATLVT012XX2565 | 47499 | TGETAGSINDNT | −0.29 | −0.53 | −1.16 |
| 3109 | ATLVT012XX782 | 45716 | NSESAGASNDNH | −1.11 | −0.01 | −0.41 |
| 3110 | ATLVT012XX1877 | 46811 | SSEAAASSNDNH | −0.89 | −0.4 | −0.31 |
| 3111 | ATLVT012XX3156 | 48090 | TSETGGASNDNT | −0.88 | 0.11 | −0.98 |
| 3112 | ATLVT01XXX1920 | 46854 | SSESAAASNDNT | −1.05 | −0.33 | −0.13 |
| 3113 | ATLVT012XX64 | 44998 | NGASAGATNDNA | −0.99 | −0.06 | −0.58 |
| 3114 | ATLVT012XX583 | 45517 | NSAAAASSNDNA | −0.98 | −0.37 | −0.21 |
| 3115 | ATLVT012XX921 | 45855 | NSQSAASSNDNT | −0.64 | −0.75 | −0.31 |
| 3116 | ATLVT012XX852 | 45786 | NSETGASTNDNT | −0.39 | −0.01 | −1.71 |
| 3117 | ATLVT012XX919 | 45853 | NSQSAASSNDNA | 0.16 | −1.06 | −1.27 |
| 3118 | ATLVT012XX223 | 45157 | NGESGASSNDNA | −0.5 | −0.97 | −0.27 |
| 3119 | ATLVT012XX2696 | 47630 | TGQTAASTNDNH | −1.11 | −0.04 | −0.43 |
| 3120 | ATLVT012XX2419 | 47353 | TGATAGSINDNA | −1.08 | −0.61 | 0.24 |
| 3121 | ATLVT012XX538 | 45472 | NGTTAASTNDNA | −1.93 | 0.21 | 0.64 |
| 3122 | ATLVT012XX1734 | 46668 | SSAAAASSNDNT | −1.51 | 0.06 | 0.11 |
| 3123 | ATLVT012XX1855 | 46789 | SSATGASTNDNA | −0.02 | −0.86 | −1.27 |
| 3124 | ATLVT012XX170 | 45104 | NGEAGAASNDNH | −0.49 | −0.77 | −0.59 |
| 3125 | ATLVT012XX2067 | 47001 | SSQSAAATNDNT | −0.73 | −1.51 | 0.75 |
| 3126 | ATLVT012XX271 | 45205 | NGETGASSNDNA | −1.2 | 0.21 | −0.65 |
| 3127 | ATLVT012XX1778 | 46712 | SSASAAATNDNH | −0.4 | −0.12 | −1.6 |
| 3128 | ATLVT012XX1949 | 46883 | SSESGASSNDNH | −0.83 | −0.36 | −0.56 |
| 3129 | ATLVT012XX676 | 45610 | NSATAAATNDNA | −0.8 | −0.84 | −0.01 |
| 3130 | ATLVT012XX352 | 45286 | NGQSAGATNDNA | −1.88 | 0.81 | −0.27 |
| 3131 | ATLVT012XX2686 | 47620 | TGQTAAASNDNA | −0.82 | −0.87 | 0.07 |
| 3132 | ATLVT012XX726 | 45660 | NSEAAAATNDNT | −2.06 | 1.72 | −1.16 |
| 3133 | ATLVT012XX2575 | 47509 | TGETGASINDNA | −1.1 | −0.88 | 0.54 |
| 3134 | ATLVT012XX2352 | 47286 | TGASAAASNDNT | −1.39 | −0.7 | 0.8 |
| 3135 | ATLVT012XX828 | 45762 | NSETAASTNDNT | −0.6 | −0.49 | −0.81 |
| 3136 | ATLVT012XX2886 | 47820 | TSAAAASSNDNT | −1.63 | 0.35 | −0.15 |
| 3137 | ATLVT012XX264 | 45198 | NGETAGSINDNT | −1.85 | 1.49 | −1.24 |
| 3138 | ATLVT012XX179 | 45113 | NGEAGASTNDNH | −0.29 | −0.49 | −1.38 |
| 3139 | ATLVT012XX344 | 45278 | NGQSAASSNDNH | −1.71 | 0.2 | 0.16 |
| 3140 | ATLVT012XX3315 | 48249 | TSTAAAATNDNT | −1.69 | 0.34 | −0.05 |
| 3141 | ATLVT012XX634 | 45568 | NSASAASINDNA | −1.37 | 0.81 | −1.2 |
| 3142 | ATLVT012XX835 | 45769 | NSETAGSSNDNA | −0.71 | −0.86 | −0.2 |
| 3143 | ATLVT012XX1446 | 46380 | SGQAAASSNDNI | −0.21 | 1.73 | 0.06 |
| 3144 | ATLVT012XX5 | 44939 | NGAAAAATNDNH | −1.66 | 0.08 | 0.2 |
| 3145 | ATLVT012XX1873 | 46807 | SSEAAAATNDNA | −2.1 | −0.02 | 1.07 |
| 3146 | ATLVT012XX2687 | 47621 | TGQTAAASNDNH | −0.96 | −1.13 | 0.54 |
| 3147 | ATLVT012XX18 | 44952 | NGAAAGAINDNT | −1.63 | −0.22 | 0.53 |
| 3148 | ATLVT012XX245 | 45179 | NGETAAATNDNH | −0.43 | −0.2 | −1.57 |
| 3149 | ATLVT012XX397 | 45331 | NGQTAGASNDNA | −1.15 | 0.11 | −0.73 |
| 3150 | ATLVT012XX2554 | 47488 | TGETAGASNDNA | −1.34 | 0.15 | −0.47 |
| 3151 | ATLVT012XX1297 | 46231 | SGEAAAATNDNA | −1.42 | −0.05 | −0.08 |
| 3152 | ATLVT012XX1984 | 46918 | SSETAGSSNDNA | −0.7 | −0.52 | −0.7 |
| 3153 | ATLVT012XX1537 | 46471 | SGQTAAATNDNA | −0.81 | −1.21 | 0.36 |
| 3154 | ATLVT012XX435 | 45369 | NGTAAAASNDNT | −1.4 | −1.03 | 1.13 |
| 3155 | ATLVT012XX208 | 45142 | NGESAGATNDNA | −1.67 | −1.22 | 1.83 |
| 3156 | ATLVT012XX1255 | 46189 | SGATAASTNDNA | −0.74 | −0.43 | −0.78 |
| 3157 | ATLVT012XX396 | 45330 | NGQTAASINDNT | −1.51 | −0.74 | 0.94 |
| 3158 | ATLVT012XX1 | 44935 | NGAAAAASNDNA | −1.57 | −0.07 | 0.16 |
| 3159 | ATLVT012XX1260 | 46194 | SGATAGASNDNT | −0.43 | −0.84 | −0.8 |
| 3160 | ATLVT012XX1871 | 46805 | SSEAAAASNDNH | −1.46 | −0.54 | 0.58 |
| 3161 | ATLVT012XX749 | 45683 | NSEAGAATNDNH | −1.39 | −0.01 | −0.23 |
| 3162 | ATLVT012XX346 | 45280 | NGQSAASINDNA | −1.95 | 0.82 | −0.35 |
| 3163 | ATLVT012XX830 | 45764 | NSETAGASNDNH | −1 | −0.49 | −0.28 |
| 3164 | ATLVT012XX3033 | 47967 | TSEAAASTNQNT | −1.36 | −0.21 | −0.04 |
| 3165 | ATLVT012XX248 | 45182 | NGETAASSNDNH | −0.64 | 0.08 | −1.65 |
| 3166 | ATLVT012XX420 | 45354 | NG?TGASTNDNT | −1.79 | −0.12 | 0.58 |
| 3167 | ATLVT012XX2561 | 47495 | TGETAGSSNDNH | −1.15 | −0.05 | −0.64 |
| 3168 | ATLVT012XX1822 | 46756 | SSATAAASNDNA | −0.63 | −0.39 | −1.1 |
| 3169 | ATLVT012XX2655 | 47589 | TGQSAGATNDNT | −1.04 | −0.36 | −0.44 |
| 3170 | ATLVT012XX3137 | 48071 | TSETAGSSNDNH | −1.16 | −0.3 | −0.32 |
| 3171 | ATLVT012XX2256 | 47190 | SSTTAAASNDNT | −0.92 | −0.83 | −0.06 |
| 3172 | ATLVT012XX3129 | 48063 | TSETAASTNDNT | −0.11 | −1.45 | −0.67 |
| 3173 | ATLVT012XX728 | 45662 | NSEAAASSNDNH | −1.66 | 0.84 | −0.95 |
| 3174 | ATLVT012XX3316 | 48250 | TSTAAASSNDNA | −0.82 | −0.52 | −0.67 |
| 3175 | ATLVT012XX3087 | 48021 | TSESAGAINDNT | −1.76 | −0.16 | 0.46 |
| 3176 | ATLVT012XX70 | 45004 | NGASAGSTNDNA | 0.93 | −0.92 | 0.01 |
| 3177 | ATLVT012XX1542 | 46476 | SGQTAASSNQNT | −1.39 | −0.74 | 0.57 |
| 3178 | ATLVT012XX1536 | 46470 | SGQTAAASNDNT | −0.83 | −1.25 | 0.26 |
| 3179 | ATLVT012XX2447 | 47381 | TGEAAAASNDNH | −0.83 | −0.63 | −0.56 |
| 3180 | ATLVT012XX975 | 45909 | NSQTAGASNDNT | −0.31 | −1.15 | −0.77 |
| 3181 | ATLVT012XX3409 | 48343 | TSTTAAATNDNA | −1.35 | 0.13 | −0.65 |
| 3182 | ATLVT012XX3027 | 47961 | TSEAAAATNDNT | −1.58 | −0.62 | 0.7 |
| 3183 | ATLVT012XX273 | 45207 | NGETGASSNDNT | 0.2 | −1.72 | −0.95 |
| 3184 | ATLVT012XX748 | 45682 | NSEAGAATNDNA | −1.89 | 0.31 | 0.02 |
| 3185 | ATLVT012XX2400 | 47334 | TGATAAASNDNT | −1.91 | −0.2 | 0.71 |
| 3186 | ATLVT012XX2458 | 47392 | TGEAAGASNDNA | −2.23 | −0.54 | 1.69 |
| 3187 | ATLVT012XX2023 | 46957 | SSQAAASTNDNA | −1.67 | −0.36 | 0.5 |
| 3188 | ATLVT012XX2991 | 47925 | TSATAGAINDNT | −1.46 | −0.38 | 0.15 |
| 3189 | ATLVT012XX3122 | 48056 | TSETAAATNDNH | −0.8 | −0.86 | −0.37 |
| 3190 | ATLVT012XX2989 | 47923 | TSATAGAINDNA | −1.48 | −0.45 | 0.26 |
| 3191 | ATLVT012XX825 | 45759 | NSETAASSNDNT | −0.75 | −0.16 | −1.39 |
| 3192 | ATLVT012XX2856 | 47790 | TGTTGAASNDNT | 1.27 | −0.18 | −0.48 |
| 3193 | ATLVT012XX256 | 45190 | NGETAGAINDNA | −1.51 | 0.35 | −0.74 |
| 3194 | ATLVT012XX3078 | 48012 | TSESAASSNDNT | −1.91 | −0.24 | 0.68 |
| 3195 | ATLVT012XX3071 | 48005 | TSESAAASNDNH | −2.07 | −0.02 | 0.67 |
| 3196 | ATLVT012XX1989 | 46923 | SSETAGSINDNT | −1.25 | −0.63 | 0.05 |
| 3197 | ATLVT012XX769 | 45703 | NSESAAASNDNA | −0.99 | −0.53 | −0.53 |
| 3198 | ATLVT012XX868 | 45802 | NSQAAAATNDNA | −1.44 | −0.67 | 0.4 |
| 3199 | ATLVT012XX1325 | 46259 | SGEAGASSNDNH | −0.68 | −0.48 | −1.15 |
| 3200 | ATLVT012XX1398 | 46332 | SGETAASSNQNT | −1.16 | −0.55 | −0.23 |
| 3201 | ATLVT012XX1854 | 46788 | SSATGASSNDNT | −0.8 | −0.56 | −0.84 |
| 3202 | ATLVT012XX181 | 45115 | NGEAGGASNDNA | −0.53 | −0.64 | −1.21 |
| 3203 | ATLVT012XX194 | 45128 | NGESAAASNDNH | −1.31 | −1.34 | 1.02 |
| 3204 | ATLVT012XX2646 | 47580 | TGQSAASSNDNT | −1.84 | −0.04 | 0.25 |
| 3205 | ATLVT012XX130 | 45064 | NGATGASTNDNA | −1.36 | −0.03 | −0.59 |
| 3206 | ATLVT012XX1360 | 46294 | SGESAGSSNDNA | −0.82 | −0.38 | −1.07 |
| 3207 | ATLVT012XX2976 | 47910 | TSATAAASNDNT | −1.22 | −0.26 | −0.55 |
| 3208 | ATLVT012XX1641 | 46575 | SGTSAASINDNT | −0.24 | −1.56 | −0.56 |
| 3209 | ATLVT012XX1852 | 46786 | SSATGASSNDNA | −0.34 | −0.9 | −1.24 |
| 3210 | ATLVT012XX2428 | 47362 | TGATGASSNDNA | −1.87 | −0.06 | 0.29 |
| 3211 | ATLVT012XX2814 | 47748 | TGTSGASSNDNT | −1.78 | 0.59 | −0.69 |
| 3212 | ATLVT012XX747 | 45681 | NSEAGAASNDNT | −0.97 | −0.76 | −0.37 |
| 3213 | ATLVT012XX3198 | 48132 | TSQAGASSNDNT | −1.12 | −0.88 | 0.05 |
| 3214 | ATLVT012XX1356 | 46290 | SGESAGASNDNT | −0.99 | −0.71 | −0.4 |
| 3215 | ATLVT012XX780 | 45714 | NSESAASTNDNT | −1.2 | 0.41 | −1.48 |
| 3216 | ATLVT012XX1890 | 46824 | SSEAAGSSNDNT | −1.09 | −0.68 | −0.29 |
| 3217 | ATLVT012XX2993 | 47927 | TSATAGSSNDNH | −1.07 | −0.5 | −0.57 |
| 3218 | ATLVT012XX52 | 44986 | NGASAAATNDNA | −1.24 | −0.55 | −0.21 |
| 3219 | ATLVT012XX2499 | 47433 | TGESAAATNDNT | −1.47 | −0.24 | −0.23 |
| 3220 | ATLVT012XX970 | 45904 | NSQTAASINDNA | −0.8 | −1.46 | 0.17 |
| 3221 | ATLVT012XX2928 | 47862 | TSASAAASNDNT | −1.11 | −0.94 | 0.03 |
| 3222 | ATLVT012XX1999 | 46933 | SSETGASTNDNA | −0.39 | −0.66 | −1.56 |
| 3223 | ATLVT012XX917 | 45851 | NSQSAAATNDNH | −0.4 | −0.9 | −1.24 |
| 3224 | ATLVT012XX874 | 45808 | NSQAAASINDNA | −1.44 | −0.36 | −0.16 |
| 3225 | ATLVT012XX2518 | 47452 | TGESGAASNDNA | −1.92 | 0.09 | 0.09 |
| 3226 | ATLVT012XX155 | 45089 | NGEAAASTNDNH | −1.08 | 0.1 | −1.39 |
| 3227 | ATLVT012XX299 | 45233 | NGQAAASINDNH | −0.06 | −1.31 | −1.31 |
| 3228 | ATLVT012XX2525 | 47459 | TGESGASSNDNH | −2.37 | −0.08 | 1.04 |
| 3229 | ATLVT012XX2980 | 47914 | TSATAASSNDNA | −0.76 | −0.6 | −1.05 |
| 3230 | ATLVT012XX1320 | 46254 | SGEAGAASNDNT | −0.64 | −1.34 | −0.3 |
| 3231 | ATLVT012XX817 | 45751 | NSETAAASNDNA | −1.48 | 0.02 | −0.62 |
| 3232 | ATLVT012XX347 | 45281 | NGQSAASINDNH | −1.45 | 0.18 | −0.87 |
| 3233 | ATLVT012XX270 | 45204 | NGETGAATNDNT | −1.68 | −1.21 | 1.3 |
| 3234 | ATLVT012XX2596 | 47530 | TGQAAASSNDNA | −1.2. | −0.66 | −0.21 |
| 3235 | ATLVT012XX1919 | 46853 | SSESAAASNDNH | −0.88 | −1.31 | 0.04 |
| 3236 | ATLVT012XX1932 | 46866 | SSESAGASNDNT | −1.82 | −0.07 | 0.04 |
| 3237 | ATLVT012XX2649 | 47583 | TGQSAASTNDNT | −1.66 | −0.21 | −0.07 |
| 3238 | ATLVT012XX409 | 45343 | NGQTGAASNDNA | −0.82 | −1.04 | −0.45 |
| 3239 | ATLVT012XX1327 | 46261 | SGEAGASINDNA | 0.57 | −2.04 | −1.56 |
| 3240 | ATLVT012XX3318 | 48252 | TSTAAASSNDNT | −0.98 | −1.01 | −0.22 |
| 3241 | ATLVT012XX1876 | 46810 | SSEAAASSNDNA | −0.66 | −0.84 | −0.99 |
| 3242 | ATLVT012XX1390 | 46324 | SGETAAASNDNA | −0.42 | −1.03 | −1.17 |
| 3243 | ATLVT012XX682 | 45616 | NSATAASINDNA | −0.91 | −1.2 | −0.1 |
| 3244 | ATLVT012XX750 | 45684 | NSEAGAATNDNT | −1.34 | −0.71 | −0.02 |
| 3245 | ATLVT012XX1733 | 46667 | SSAAAASSNDNH | −1.97 | 0.64 | −0.67 |
| 3246 | ATLVT012XX697 | 45631 | NSATGAASNDNA | −0.65 | −1.7 | 0.06 |
| 3247 | ATLVT012XX1302 | 46236 | SGEAAASSNDNT | −1.94 | −2.06 | 2.73 |
| 3248 | ATLVT012XX2128 | 47062 | SSQTAGSSNDNA | −0.57 | −1.5 | −0.34 |
| 3249 | ATLVT012XX1975 | 46909 | SSETAASTNDNA | −1.85 | −1.44 | 1.78 |
| 3250 | ATLVT012XX741 | 45675 | NSEAAGSSNDNT | −2.42 | 1.13 | −0.57 |
| 3251 | ATLVT012XX196 | 45130 | NGESAAATNDNA | −2.11 | −0.74 | 1.31 |
| 3252 | ATLVT012XX2430 | 47364 | TGATGASSNDNT | −2.53 | 0.32 | 0.64 |
| 3253 | ATLVT012XX2481 | 47415 | TGEAGASINDNT | −0.71 | −1.41 | −0.26 |
| 3254 | ATLVT012XX57 | 44991 | NGASAASSNDNT | −1.14 | −0.96 | −0.1 |
| 3255 | ATLVT012XX3105 | 48039 | TSESGASTNDNT | −1.95 | −0.36 | 0.51 |
| 3256 | ATLVT012XX598 | 45532 | NSAAAGSTNDNA | −1.59 | −0.13 | −0.42 |
| 3257 | ATLVT012XX632 | 45566 | NSASAASSNDNH | −1.43 | −0.69 | 0.03 |
| 3258 | ATLVT012XX1304 | 46238 | SGEAAASTNQNH | −0.48 | −1.06 | −1.12 |
| 3259 | ATLVT012XX3168 | 48102 | TSQAAAASNDNT | −1.43 | −1.16 | 0.6 |
| 3260 | ATLVT012XX3131 | 48065 | TSETAGASNDNH | −1.48 | −0.47 | −0.21 |
| 3261 | ATLVT012XX2466 | 47400 | TGEAAGSSNDNT | −2 | 0.14 | −0.14 |
| 3262 | ATLVT012XX3085 | 48019 | TSESAGATNDNA | −2.36 | 0.3 | 0.28 |
| 3263 | ATLVT012XX625 | 45559 | NSASAAASNDNA | −0.08 | −1.58 | 1.24 |
| 3264 | ATLVT012XX1354 | 46288 | SGESAGASNDNA | −1 | −0.81 | −0.72 |
| 3265 | ATLVT012XX266 | 45200 | NGETGAASNDNH | −1.51 | −0.87 | 0.22 |
| 3266 | ATLVT012XX2115 | 47049 | SSQTAAATNDNT | −2.32 | −1.01 | 1.8 |
| 3267 | ATLVT012XX2691 | 47625 | TGQTAAATNDNT | −0.96 | −1.2 | −0.32 |
| 3268 | ATLVT012XX3222 | 48156 | TSQSAASSNDNT | −0.99 | −1.2 | −0.31 |
| 3269 | ATLVT012XX3141 | 48075 | TSETAGSINDNT | −1.02 | −1.17 | −0.28 |
| 3270 | ATLVT012XX201 | 45135 | NGESAASSNDNT | −2.35 | −1.58 | 2.51 |
| 3271 | ATLVT012XX978 | 45912 | NSQTAGAINDNT | −0.69 | −1.11 | −0.94 |
| 3272 | ATLVT012XX2429 | 47363 | TGATGASSNDNH | −2.25 | 0.27 | −0.06 |
| 3273 | ATLVT012XX211 | 45145 | NGESAGSSNDNA | −1.86 | −0.21 | −0.12 |
| 3274 | ATLVT012XX1348 | 46282 | SGESAASSNDNA | −1.2 | −1.46 | 0.34 |
| 3275 | ATLVT012XX204 | 45138 | NGESAASTNDNT | −1.45 | −1.29 | 0.55 |
| 3276 | ATLVT012XX706 | 45640 | NSATGASTNDNA | −1.49 | 0.55 | −1.78 |
| 3277 | ATLVT012XX3084 | 48018 | TSESAGASNDNT | −2.16 | −0.32 | 0.5 |
| 3278 | ATLVT012XX165 | 45099 | NGEAAGSSNDNT | −1.82 | −0.34 | −0.07 |
| 3279 | ATLVT012XX2001 | 46935 | SSETGASTNDNT | −0.51 | −1.29 | −1.09 |
| 3280 | ATLVT012XX1465 | 46399 | SGQAGAATNDNA | −1.77 | −0.9 | 0.55 |
| 3281 | ATLVT012XX282 | 45216 | NGETGGATNDNT | −2.11 | −0.72 | 0.9 |
| 3282 | ATLVT012XX100 | 45034 | NGATAAATNDNA | −1.08 | −0.34 | −1.38 |
| 3283 | ATLVT012XX2520 | 47454 | TGESGAASNDNT | −1.99 | −0.34 | 0.15 |
| 3284 | ATLVT012XX415 | 45349 | NGQTGASSNDNA | −1.89 | −0.76 | 0.54 |
| 3285 | ATLVT012XX2478 | 47412 | TGEAGASSNDNT | −1.38 | −0.65 | −0.49 |
| 3286 | ATLVT012XX754 | 45688 | NSEAGASINDNA | −1.68 | −0.46 | −0.27 |
| 3287 | ATLVT012XX6 | 44940 | NGAAAAATNDNT | −2.07 | −0.38 | 0.29 |
| 3288 | ATLVT012XX3174 | 48108 | TSQAAASSNDNI | −1.29 | −1.58 | 0.49 |
| 3289 | ATLVT012XX261 | 45195 | NGETAGSSNDNT | −2.05 | 1.34 | −1.98 |
| 3290 | ATLVT012XX387 | 45321 | NGQTAAASNDNT | −0.74 | −1.6 | −0.44 |
| 3291 | ATLVT012XX2653 | 47587 | TGQSAGATNDNA | −1.36 | −0.73 | −0.51 |
| 3292 | ATLVT01XXX1329 | 46263 | SGEAGASINDNT | −0.32 | −1.71 | −1.04 |
| 3293 | ATLVT012XX72 | 45006 | NGASAGSINDNT | −2.11 | −0.87 | 0.95 |
| 3294 | ATLVT012XX1295 | 46229 | SGEAAAASNDNH | −2.29 | −1.35 | 1.87 |
| 3295 | ATLVT012XX171 | 45105 | NGEAGAASNDNT | −1.3 | −0.82 | −0.51 |
| 3296 | ATLVT012XX411 | 45345 | NGQTGAASNDNT | −1.25 | −0.78 | −0.66 |
| 3297 | ATLVT012XX636 | 45570 | NSASAASINDNT | −1.16 | −1.05 | −0.46 |
| 3298 | ATLVT012XX3121 | 48055 | TSETAAATNDNA | −1.28 | −1.09 | −0.26 |
| 3299 | ATLVT012XX3123 | 48057 | TSETAAATNDNT | −2.1 | −0.62 | 0.52 |
| 3300 | ATLVT012XX253 | 45187 | NGETAGASNDNA | −1.93 | 0.33 | −0.98 |
| 3301 | ATLVT012XX3150 | 48084 | TSETGASSNDNT | −0.72 | −1.48 | −0.73 |
| 3302 | ATLVT012XX2883 | 47817 | TSAAAAATNDNT | −2 | −0.44 | 0.12 |
| 3303 | ATLVT012XX3166 | 48100 | TSQAAAASNDNA | −1.46 | −0.94 | −0.17 |
| 3304 | ATLVT012XX1874 | 46808 | SSEAAAATNDNH | −1.94 | −0.14 | −0.37 |
| 3305 | ATLVT012XX386 | 45320 | NGQTAAASNDNH | −0.9 | −1.32 | −0.66 |
| 3306 | ATLVT012XX341 | 45275 | NGQSAAATNDNH | −1.33 | −0.21 | −1.36 |
| 3307 | ATLVT012XX2527 | 47461 | TGESGASTNDNA | −2.26 | −0.24 | 0.26 |
| 3308 | ATLVT012XX850 | 45784 | NSETGASTNDNA | −1.39 | −0.16 | −1.35 |
| 3309 | ATLVT012XX388 | 45322 | NGQTAAAINDNA | −1.56 | −0.55 | −0.56 |
| 3310 | ATLVT012XX186 | 45120 | NGEAGGATNDNT | −0.89 | −0.7 | −1.54 |
| 3311 | ATLVT012XX1423 | 46357 | SGETGASTNDNA | −1.17 | −0.95 | −0.72 |
| 3312 | ATLVT012XX826 | 45760 | NSETAASTNDNA | −1.03 | −1.01 | −0.91 |
| 3313 | ATLVT012XX3170 | 48104 | TSQAAAATNDNH | −1.56 | −0.89 | −0.17 |
| 3314 | ATLVT012XX840 | 45774 | NSETAGSTNDNT | −0.92 | −1.22 | −0.85 |
| 3315 | ATLVT012XX348 | 45282 | NGQSAASINDNT | −1.99 | 0.78 | −1.6 |
| 3316 | ATLVT012XX2355 | 47289 | TGASAAATNDNT | −2.1 | −0.47 | 0.16 |
| 3317 | ATLVT012XX773 | 45707 | NSESAAATNDNH | −1.37 | −0.31 | −1.29 |
| 3318 | ATLVT012XX3118 | 48052 | TSETAAASNDNA | −0.98 | −1.39 | −0.58 |
| 3319 | ATLVT012XX1249 | 46183 | SGATAAATNDNA | −0.7 | −1.09 | −1.46 |
| 3320 | ATLVT012XX730 | 45664 | NSEAAASTNDNA | −2.63 | 0.17 | 0.21 |
| 3321 | ATLVT012XX1827 | 46761 | SSATAAATNDNT | −1.08 | −1.25 | −0.64 |
| 3322 | ATLVT012XX3102 | 48036 | TSESGASSNDNT | −2.3 | −1.02 | 1.1 |
| 3323 | ATLVT012XX340 | 45274 | NGQSAAATNDNA | −0.79 | −1.07 | 1.43 |
| 3324 | ATLVT012XX630 | 45564 | NSASAAATNDNT | −1.04 | −1.26 | −0.77 |
| 3325 | ATLVT012XX976 | 45910 | NSQTAGATNDNA | −1.2 | −1.13 | −0.68 |
| 3326 | ATLVT012XX198 | 45132 | NGESAAATNDNT | −1.66 | −1.73 | 0.88 |
| 3327 | ATLVT012XX1439 | 46373 | SGQAAAASNDNH | −2.37 | −0.83 | 0.92 |
| 3328 | ATLVT012XX2254 | 47188 | SSTTAAASNDNA | −1.86 | 0.57 | −1.79 |
| 3329 | ATLVT012XX2162 | 47096 | SSTAAAAINDNH | −2.04 | −0.2 | −0.5 |
| 3330 | ATLVT012XX916 | 45850 | NSQSAAATNDNA | −1.47 | −0.23 | −1.45 |
| 3331 | ATLVT012XX2015 | 46949 | SSQAAAASNDNH | −1.88 | −1.32 | 0.67 |
| 3332 | ATLVT012XX869 | 45803 | NSQAAAATNDNH | −1.57 | −0.63 | −0.75 |
| 3333 | ATLVT012XX3171 | 48105 | TSQAAAATNDNT | −1.44 | −1.52 | 0.17 |
| 3334 | ATLVT012XX2690 | 47624 | TGQTAAATNDNH | −1.29 | −0.8 | −1.03 |
| 3335 | ATLVT012XX2737 | 47671 | TGTAAAATNDNA | −2.27 | −1.19 | 1.14 |
| 3336 | ATLVT012XX1392 | 46326 | SGETAAASNDNT | −0.75 | −1.34 | −1.27 |
| 3337 | ATLVT012XX2881 | 47815 | TSAAAAATNDNA | −2.15 | −0.1 | −0.46 |
| 3338 | ATLVT012XX2161 | 47095 | SSTAAAATNDNA | −2 | −1.45 | 1.02 |
| 3339 | ATLVT012XX1350 | 46284 | SGESAASSNDNT | −1.52 | −1.32 | 0 |
| 3340 | ATLVT012XX3138 | 48072 | TSETAGSSNDNT | −1.54 | −0.82 | −0.72 |
| 3341 | ATLVT012XX2431 | 47365 | TGATGASTNDNA | −1.75 | −0.38 | −0.95 |
| 3342 | ATLVT012XX355 | 45289 | NGQSAGSSNDNA | −2.02 | −0.84 | 0.09 |
| 3343 | ATLVT012XX1009 | 45943 | NSTAAAASNDNA | −1.35 | −1.03 | −0.81 |
| 3344 | ATLVT012XX771 | 45705 | NSESAAASNDNT | −0.91 | −1.83 | −0.54 |
| 3345 | ATLVT012XX148 | 45082 | NGEAAAATNDNA | −0.96 | −0.94 | −1.63 |
| 3346 | ATLVT012XX1203 | 46137 | SGASAAATNDNT | −1.39 | −1.29 | −0.44 |
| 3347 | ATLVT012XX3130 | 48064 | TSETAGASNDNA | −1.8 | −0.94 | −0.2 |
| 3348 | ATLVT012XX1374 | 46308 | SGESGASSNDNT | −1.54 | −1.02 | −0.57 |
| 3349 | ATLVT012XX819 | 45753 | NSETAAASNDNT | −0.23 | −2.46 | −0.97 |
| 3350 | ATLVT012XX2878 | 47812 | ISAAAAASNDNA | −2.01 | −0.68 | −0.24 |
| 3351 | ATLVT012XX1444 | 46378 | SGQAAASSNDNA | −0.84 | −1.66 | −1.01 |
| 3352 | ATLVT012XX1974 | 46908 | SSETAASSNDNT | −2.45 | −1.31 | 1.3 |
| 3353 | ATLVT012XX484 | 45418 | NGTSAAATNDNA | −1.83 | −0.98 | −0.21 |
| 3354 | ATLVT012XX2479 | 47413 | TGEAGASINDNA | −1.22 | −1.08 | −1.18 |
| 3355 | ATLVT012XX3114 | 48048 | TSESGGSSNDNT | −2.07 | −1.2 | 0.44 |
| 3356 | ATLVT012XX3007 | 47941 | ISATGASTNDNA | −1.67 | −0.42 | 1.28 |
| 3357 | ATLVT012XX3031 | 47965 | TSEAAASTNDNA | −1.79 | −1.17 | −0.1 |
| 3358 | ATLVT012XX2506 | 47440 | TGESAGASNDNA | −2.72 | −0.61 | 0.77 |
| 3359 | ATLVT012XX106 | 45040 | NGATAASTNDNA | −1.65 | −0.83 | −0.81 |
| 3360 | ATLVT012XX2382 | 47316 | TGASGASSNDNT | −2.24 | −0.24 | −0.55 |
| 3361 | ATLVT012XX731 | 45665 | NSEAAASTNDNH | −2.45 | 0.42 | −1.08 |
| 3362 | ATLVT012XX267 | 45201 | NGETGAASNDNT | −2.04 | −0.84 | −0.18 |
| 3363 | ATLVT012XX1438 | 46372 | SGQAAAASNDNA | −2.73 | 0.13 | −0.23 |
| 3364 | ATLVT012XX2644 | 47578 | TGQSAASSNDNA | −2.03 | −0.73 | −0.35 |
| 3365 | ATLVT012XX3125 | 48059 | TSETAASSNDNH | −1.56 | −1.12 | −0.64 |
| 3366 | ATLVT012XX2014 | 46948 | SSQAAAASNDNA | −2.31 | −1.04 | 0.55 |
| 3367 | ATLVT012XX7 | 44941 | NGAAAASSNDNA | −1.9 | −0.84 | −0.44 |
| 3368 | ATLVT012XX866 | 45800 | NSQAAAASNDNH | −1.89 | −0.92 | −0.38 |
| 3369 | ATLVT012XX973 | 45907 | NSQTAGASNDNA | −1.55 | −1.37 | −0.38 |
| 3370 | ATLVT012XX1923 | 46857 | SSESAAATNDNT | −1.88 | −1.16 | −0.1 |
| 3371 | ATLVT012XX867 | 45801 | NSQAAAASNDNT | −1.49 | −1.31 | −0.58 |
| 3372 | ATLVT012XX265 | 45199 | NGETGAASNDNA | −2.04 | −0.98 | −0.08 |
| 3373 | ATLVT012XX225 | 45159 | NGESGASSNDNT | −1.43 | −1.42 | −0.57 |
| 3374 | ATLVT012XX2542 | 47476 | TGETAAASNDNA | −1.1 | −1.41 | −1.16 |
| 3375 | ATLVT012XX178 | 45112 | NGEAGASTNDNA | −1.5 | −0.54 | −1.59 |
| 3376 | ATLVT012XX2446 | 47380 | TGEAAAASNDNA | −1.81 | −0.45 | −1.19 |
| 3377 | ATLVT012XX418 | 45352 | NGQTGASTNDNA | −2.52 | −1.12 | 0.89 |
| 3378 | ATLVT012XX3167 | 48101 | TSQAAAASNDNH | −2.09 | −0.88 | −0.14 |
| 3379 | ATLVT012XX1300 | 46234 | SGEAAASSNDNA | −1.45 | −1.26 | −0.77 |
| 3380 | ATLVT012XX268 | 45202 | NGETGAATNDNA | −2.69 | −0.87 | 0.83 |
| 3381 | ATLVT012XX834 | 45768 | NSETAGATNDNT | −1.89 | −0.89 | −0.52 |
| 3382 | ATLVT012XX2926 | 47860 | TSASAAASNDNA | −1.53 | −1.4 | −0.5 |
| 3383 | ATLVT012XX390 | 45324 | NGQTAAATNDNT | −1.67 | −0.9 | −0.95 |
| 3384 | ATLVT012XX2550 | 47484 | TGETAASSNQNT | −1.31 | −1.6 | −0.67 |
| 3385 | ATLVT012XX3127 | 48061 | TSETAASTNDNA | −1.2 | −1.61 | −0.85 |
| 3386 | ATLVT012XX1727 | 46661 | SSAAAAASNDNH | −2.0] | −0.5 | −0.91 |
| 3387 | ATLVT012XX2560 | 47494 | TGETAGSSNDNA | −1.41 | −0.96 | −1.41 |
| 3388 | ATLVT012XX745 | 45679 | NSEAGAASNDNA | −1.73 | −1.35 | −0.38 |
| 3389 | ATLVT012XX154 | 45088 | NGEAAASTNDNA | −1.93 | −0.47 | −1.18 |
| 3390 | ATLVT012XX1342 | 46276 | SGESAAASNDNA | −1.11 | −1.49 | −1.29 |
| 3391 | ATLVT012XX147 | 45081 | NGEAAAASNDNT | −1.36 | −1.61 | −0.71 |
| 3392 | ATLVT012XX2529 | 47463 | TGESGASTNDNT | −2.54 | −1.52 | 1.18 |
| 3393 | ATLVT012XX1779 | 46713 | SSASAAATNDNT | −1.88 | −1.26 | −0.31 |
| 3394 | ATLVT012XX2526 | 47460 | TGESGASSNDNT | −2.9 | −1.45 | 1.67 |
| 3395 | ATLVT012XX433 | 45367 | NGTAAAASNDNA | −1.41 | −2.11 | −0.03 |
| 3396 | ATLVT012XX1534 | 46468 | SGQTAAASNDNA | −1.18 | −1.3 | −1.49 |
| 3397 | ATLVT012XX822 | 45756 | NSETAAATNDNT | −1.14 | −1.9 | −0.8 |
| 3398 | ATLVT012XX913 | 45847 | NSQSAAASNDNA | −1.43 | −1.4 | −1 |
| 3399 | ATLVT012XX434 | 45368 | NGTAAAASNDNH | −3.01 | −0.33 | 0.33 |
| 3400 | ATLVT012XX1872 | 46806 | SSEAAAASNDNT | −1.71 | −1.7 | −0.17 |
| 3401 | ATLVT012XX275 | 45209 | NGETGASTNDNH | 2.11 | −0.75 | −0.73 |
| 3402 | ATLVT012XX1870 | 46804 | SSEAAAASNDNA | −1.73 | −1.44 | −0.51 |
| 3403 | ATLVT01XXX1326 | 46260 | SGEAGASSNDNT | −1.21 | −1.87 | −0.9 |
| 3404 | ATLVT012XX258 | 45192 | NGETAGATNDNT | −1.99 | −0.81 | −0.91 |
| 3405 | ATLVT012XX580 | 45514 | NSAAAAATNDNA | −1.74 | −1.24 | −0.81 |
| 3406 | ATLVT012XX1921 | 46855 | SSESAAATNDNA | −1.45 | −0.91 | −1.75 |
| 3407 | ATLVT012XX721 | 45655 | NSEAAAASNDNA | −2.21 | −0.49 | −0.99 |
| 3408 | ATLVT012XX2163 | 47097 | SSTAAAATNDNT | −2.42 | −1.18 | 0.23 |
| 3409 | ATLVT012XX2559 | 47493 | TGETAGAINDNT | −1.65 | −1.1 | −1.2 |
| 3410 | ATLVT012XX2257 | 47191 | SSTTAAATNDNA | −1.74 | −0.65 | −1.65 |
| 3411 | ATLVT012XX2557 | 47491 | TGETAGATNDNA | −1.74 | −1.11 | −1.08 |
| 3412 | ATLVT012XX3124 | 48058 | TSETAASSNDNA | −2.04 | −1.74 | 0.25 |
| 3413 | ATLVT012XX918 | 45852 | NSQSAAATNDNT | −2.13 | −1.15 | −0.42 |
| 3414 | ATLVT012XX54 | 44988 | NGASAAATNDNT | 2.05 | −1.17 | −0.55 |
| 3415 | ATLVT012XX722 | 45656 | NSEAAAASNDNH | −1.95 | −0.91 | −1.07 |
| 3416 | ATLVT012XX69 | 45003 | NGASAGSSNDNT | −1.73 | −1.15 | −1.14 |
| 3417 | ATLVT012XX770 | 45704 | NSESAAASNDNH | −1.6 | 1.56 | −0.84 |
| 3418 | ATLVT012XX2694 | 47628 | TGQTAASSNDNT | −2.07 | −1.21 | −0.51 |
| 3419 | ATLVT012XX436 | 45370 | NGTAAAATNDNA | −3.27 | −1.99 | 2.49 |
| 3420 | ATLVT012XX2562 | 47496 | TGETAGSSNDNT | −2.35 | −0.27 | −1.37 |
| 3421 | ATLVT012XX410 | 45344 | NGQTGAASNDNH | −2.35 | −1.31 | −0.04 |
| 3422 | ATLVT012XX4 | 44938 | NGAAAAATNDNA | −2.35 | −1.1 | −0.34 |
| 3423 | ATLVT012XX727 | 45661 | NSEAAASSNDNA | −2.37 | −0.36 | −1.27 |
| 3424 | ATLVT012XX3312 | 48246 | ISTAAAASNDNT | −2.28 | −0.94 | −0.69 |
| 3425 | ATLVT012XX582 | 45516 | NSAAAAATNDNT | −1.58 | −2.08 | −0.44 |
| 3426 | ATLVT012XX274 | 45208 | NGETGASTNDNA | −3.19 | 0.61 | −1.24 |
| 3427 | ATLVT012XX226 | 45160 | NGESGASTNDNA | −1.9 | −1.22 | 11.4 |
| 3428 | ATLVT012XX739 | 45673 | NSEAAGSSNDNA | −2.14 | −0.93 | −1.42 |
| 3429 | ATLVT012XX3135 | 48069 | TSETAGAINDNT | −2.34 | −1.15 | −0.8 |
| 3430 | ATLVT012XX243 | 45177 | NGETAAASNDNT | −1.42 | −1.81 | −1.55 |
| 3431 | ATLVT012XX3126 | 48060 | TSETAASSNDNT | −1.79 | −1.69 | −1.12 |
| 3432 | ATLVT012XX723 | 45657 | NSEAAAASNDNT | −2.29 | −0.44 | −1.87 |
| 3433 | ATLVT012XX772 | 45706 | NSESAAATNDNA | −1.94 | −1 | −1.77 |
| 3434 | ATLVT012XX1728 | 46662 | SSAAAAASNDNT | −1.48 | −2.22 | −1.15 |
| 3435 | ATLVT012XX2158 | 47092 | SSTAAAASNDNA | −2.24 | −1.06 | −1.34 |
| 3436 | ATLVT012XX2259 | 47193 | SSTTAAATNDNT | −2.6 | −0.83 | −1.18 |
| 3437 | ATLVT012XX180 | 45114 | NGEAGASINDNT | −2.14 | −0.86 | −1.95 |
| 3438 | ATLVT012XX3075 | 48009 | TSESAAATNDNT | −2.7 | −0.99 | −0.83 |
| 3439 | ATLVT012XX1296 | 46230 | SGEAAAASNDNT | −2.07 | −1.64 | −1.J |
| 3440 | ATLVT012XX246 | 45180 | NGETAAATNDNT | −2.07 | −1.6 | −1.23 |
| 3441 | ATLVT012XX9 | 44943 | NGAAAASSNDNT | −2.67 | −1.3 | −0.65 |
| 3442 | ATLVT012XX729 | 45663 | NSEAAASSNDNT | −1.9 | −1.67 | −1.58 |
| 3443 | ATLVT012XX228 | 45162 | NGESGASTNDNT | −1.96 | −1.64 | −1.83 |
| 3444 | ATLVT012XX2160 | 47094 | SSTAAAASNDNT | −2.35 | −1.83 | −0.95 |
| 3445 | ATLVT012XX1294 | 46228 | SGEAAAASNDNA | −2.69 | −1.12 | −1.38 |
| 3446 | ATLVT012XX241 | 45175 | NGETAAASNDNA | −2.1 | −2.14 | −1.24 |
| 3447 | ATLVT012XX1731 | 46665 | SSAAAAATNDNT | −3.05 | −1.66 | −0.32 |
| 3448 | ATLVT012XX145 | 45079 | NGEAAAASNDNA | −1.94 | −2.05 | −1.73 |
| 3449 | ATLVT012XX774 | 45708 | NSESAAATNDNT | −2.48 | −1.63 | −1.39 |
| 3450 | ATLVT012XX1729 | 46663 | SSAAAAATNDNA | −2.72 | −1.69 | −1.22 |
| 3451 | ATLVT012XX146 | 45080 | NGEAAAASNDNH | −2.29 | −2.2 | −1.71 |
| 3452 | ATLVT012XX276 | 45210 | NGETGASTNDNT | −2.78 | −2.23 | −1.02 |
| 3453 | ATLVT012XX244 | 45178 | NGETAAATNDNA | −2.4 | −1.95 | −2.26 |
| 3454 | ATLVT012XX1726 | 46660 | SSAAAAASNDNA | −2.73 | −2.2 | −1.45 |
| 3455 | ATLVT012XX385 | 45319 | NGQTAAASNDNA | −2.8 | −2.03 | −1.58 |
| 3456 | ATLVT012XX865 | 45799 | NSQAAAASNDNA | −2.98 | −2.28 | 1.48 |

LENGTHY TABLES
The patent application contains a lengthy table section. A copy of the table is available in electronic form from the USPTO web site (https://seqdata.uspto.gov/docdetail?docId=US20260062450A1). An electronic copy of the table will also be available from the USPTO upon request and payment of the fee set forth in 37 CFR 1.19(b)(3).

What is claimed is:

1. A modified adeno-associated virus (AAV) capsid protein, comprising:
(i) a targeting peptide within VR VIII; or
(ii) a peptide segment within VR I,
wherein the targeting peptide has a sequence of X₁X₂X₃RGDX₇X₈X₉X₁₀, wherein X₁, X₂, X₃, X₇, X₈, X₉ and X₁₀ are independently selected from any amino acid residue, and wherein the peptide segment has an amino acid sequence of P₁P₂P₃P₄P₅P₆P₇P₈NDNP₁₂ and P₁, P₂, P₃, P₄, P₅, P₆, P₇, P₈, and P₁₂ are independently selected from any amino acid residue.

2. The modified AAV capsid protein of claim 1, comprising: (i) the targeting peptide within VR VIII; and (ii) the peptide segment within VR I.

3. The modified AAV capsid protein of claim 1 or 2, wherein targeting peptide does not comprise RGDLLLS (SEQ ID NO: 1).

4. The modified AAV capsid protein of claim 1 or 2, wherein the peptide segment does not comprise an alanine (A) at P₆ and a threonine (T) at P₈.

5. The modified AAV capsid protein of claim 1 or 2, wherein
the targeting peptide does not comprise RGDLLLS (SEQ ID NO: 1); and
the peptide segment does not comprise an alanine (A) at P₆ and a threonine (T) at P₈.

6. The modified AAV capsid protein of any one of claims 1-5, wherein the modified AAV capsid protein has one or more modifications comprising amino acid insertions, deletions, substitutions, or combinations thereof as compared to a reference AAV capsid protein.

7. The modified AAV capsid protein of claim 6, wherein the modified AAV capsid protein comprises one or more modifications comprising an amino acid insertion, deletion, substitution, or a combination thereof as compared to a reference AAV capsid protein to introduce the targeting peptide within VR VIII.

8. The modified AAV capsid protein of any one of claims 6-7, further comprising one or more modifications outside of the VR I and VR VIII of the reference AAV capsid protein.

9. The modified AAV capsid protein of any one of claims 6-8, having at least 90%, 95%, 98%, 99% or 99.5% sequence identity to the sequence of the reference AAV capsid protein.

10. The modified AAV capsid protein of any one of claims 1-9, wherein the AAV capsid protein is selected from VP1, VP2 and VP3.

11. The modified AAV capsid protein of any one of claims 6-10, wherein the reference AAV capsid protein is a capsid protein of an AAV variant selected from the group consisting of: AAV9; Anc8065; Anc80-55; Anc80-129; Anc80-156; Anc80-751; Anc80-1029; Anc80-1712; AAV2; AAV1; AAV6; AAV3; AAV LK03; AAV7; AAV8; AAV hu.37; AAV rh.10; AAV hu.68; AAV10; AAV5; AAV3-3; AAV4-4; AAV1-A; hu.46-A; hu.48-A; hu.44-A; hu.43-A; AAV6-A; hu.34-B; hu.47-B; hu.29-B; rh.63-B; hu.56-B; hu.45-B; rh.57-B; rh.35-B; rh.58-B; rh.28-B; rh.51-B; rh.19-B; rh.49-B; rh.52-B; rh.13-B; AAV2-B; rh.20-B; rh.24-B; rh.64-B; hu.27-B; hu.21-B; hu.22-B; hu.23-B; hu.7-C; hu.61-C; rh.56-C; hu.9-C; hu.54-C; hu.53-C; hu.60-C; hu.55-C; hu.2-C; hu.1-C; hu.18-C; hu.3-C; hu.25-C; hu.15-C; hu.16-C; hu.11-C; hu.10-C; hu.4-C; rh.54-D; rh.48-D; rh.55-D; rh.62-D; AAV7-D; rh.52-E; rh.51-E; hu.39-E; rh.53-E; hu.37-E; rh.43-E; rh.50-E; rh.49-E; rh.61-E; hu.41-E; rh.64-E; rh74; hu.42-E; rh.57-E; rh.40-E; hu.67-E; hu.17-E; hu.6-E; hu.66-E; rh.38-E; hu.32-F; AAV9/hu; hu.31-F; Anc80; Anc81; Anc82; Anc83; Anc84; Anc94; Anc113; Anc126; Anc127; Anc80L27; Anc80L59; Anc80L60; Anc80L62; Anc80L33; Anc80L36; Anc80L44; Anc80L1; Anc110; and Anc80DI.

12. The modified AAV capsid protein of any one of claims 6-11, wherein the reference AAV capsid protein is a capsid protein having a sequence selected from SEQ ID Nos: 54-152, 44885-44898, 44916-44917, or a fragment thereof.

13. The modified AAV capsid protein of any one of 6-12, wherein the reference AAV capsid protein is a capsid protein having a sequence of SEQ ID NO: 61 or a fragment thereof.

14. The modified AAV capsid protein of any one of claims 6-12, wherein the reference AAV capsid protein is a capsid protein having a sequence of SEQ ID NO: 132 or a fragment thereof.

15. The modified AAV capsid protein of any one of claims 6-12, the reference AAV capsid protein is a capsid protein having a sequence of SEQ ID NO: 142 or a fragment thereof.

16. The modified AAV capsid protein of any one of claims 6-12, wherein the reference AAV capsid protein is a capsid protein selected from: Anc80-55, Anc80-129, Anc80-156, Anc80-751, Anc80-1029, and Anc80-1712.

17. The modified AAV capsid protein of claim 16, wherein the reference AAV capsid protein is a capsid protein having a sequence selected from SEQ ID NOs: 44885-44898, and 44916-44917.

18. The modified AAV capsid protein of any one of claims 1-10, having the sequence selected from SEQ ID NOs.: 44900-44909.

19. The modified AAV capsid protein of any one of claims 1-18, wherein X₇, X₈, X₉ and X₁₀ are independently selected from A, D, E, F, G, H, I, K, L, M, N, Q, R, S, T, V, and Y.

20. The modified AAV capsid protein of any one of claims 1-19, wherein X₁, X₂, and X₃ are independently selected from any amino acid residue.

21. The modified AAV capsid protein of claim 20, wherein X₁, X₂, and X₃ are amino acids identical to the amino acids at corresponding positions of the reference AAV capsid protein.

22. The modified AAV capsid protein of claim 20, wherein X₁ is an amino acid that is identical to the amino acid at a corresponding position of the reference AAV capsid protein.

23. The modified AAV capsid protein of claim 20, wherein X₂ is an amino acid that is identical to the amino acid at a corresponding position of the reference AAV capsid protein.

24. The modified AAV capsid protein of claim 20, wherein X₃ is an amino acid that is identical to the amino acid at a corresponding position of the reference AAV capsid protein.

25. The modified AAV capsid protein of claim 20, wherein X₁ and X₃ are amino acids that are identical to the amino acids at corresponding positions of the reference AAV capsid protein.

26. The modified AAV capsid protein of claim 20, wherein X₁ and X₂ are amino acids that are identical to the amino acids at corresponding positions of the reference AAV capsid protein.

27. The modified AAV capsid protein of claim 20, wherein X₂ and X₃ are amino acids that are identical to the amino acids at corresponding positions of the reference AAV capsid protein.

28. The modified AAV capsid protein of claim 20, wherein X₁, X₂, and X₃ are amino acids that are not identical to the amino acids at corresponding positions of the reference AAV capsid protein.

29. The modified AAV capsid protein of claim 20, wherein X₁ is an amino acid that is not identical to the amino acid at a corresponding position of the reference AAV capsid protein.

30. The modified AAV capsid protein of claim 20, wherein X₂ is an amino acid that is not identical to the amino acid at a corresponding position of the reference AAV capsid protein.

31. The modified AAV capsid protein of claim 20, wherein X₃ is an amino acid that is not identical to the amino acid at a corresponding position of the reference AAV capsid protein.

32. The modified AAV capsid protein of claim 20, wherein X₁ and X₃ are amino acids that are not identical to the amino acids at corresponding positions of the reference AAV capsid protein.

33. The modified AAV capsid protein of claim 20, wherein X₁ and X₂ are amino acids that are not identical to the amino acids at corresponding positions of the reference AAV capsid protein.

34. The modified AAV capsid protein of claim 20, wherein X₂ and X₃ are amino acids that are not identical to the amino acids at corresponding positions of the reference AAV capsid protein.

35. The modified AAV capsid protein of any one of claims 1-34, wherein the modified AAV capsid protein comprises one or more substitutions, one or more insertions, one or more deletions, or a combination thereof into VR VIII of the reference AAV capsid protein.

36. The modified AAV capsid protein of claim 35, wherein one or more modifications comprises a substitution of one or more amino acids between amino acid positions 565 and 595 of the reference AAV capsid.

37. The modified AAV capsid protein of any one of claims 1-36, wherein X₁ is selected from S, E, A, D, N, Q, or T.

38. The modified AAV capsid protein of claim 37, wherein X₁ is D or E.

39. The modified AAV capsid protein of claim 37, wherein X₁ is S, A or T.

40. The modified AAV capsid protein of claim 37, wherein X₁ is S, A or E.

41. The modified AAV capsid protein of any one of claims 1-36, wherein X₂ is selected from N, A, D, E, F, G, H, I, K, L, M, N, P, Q, R, S, T, V, W, or Y.

42. The modified AAV capsid protein of claim 41, wherein X₂ is selected from K, E, D, A, S, F, N, V or L.

43. The modified AAV capsid protein of claim 41, wherein X₂ is selected from K, E or D.

44. The modified AAV capsid protein of claim 41, wherein X₂ is selected from N, A or Y.

45. The modified AAV capsid protein of claim 41, wherein X₂ is selected from N, Y or S.

46. The modified AAV capsid protein of any one of claims 1-45, wherein X₃ is selected from R, Q, A, D, E, F, G, H, I, K, L, M, N, P, S, T, V, W, or Y.

47. The modified AAV capsid protein of claim 46, wherein X₃ is selected from Y, V or F.

48. The modified AAV capsid protein of claim 46, wherein X₃ is selected from N, A, Y or S.

49. The modified AAV capsid protein of claim 46, wherein X₃ is selected from I, Q, R, V, T or M.

50. The modified AAV capsid protein of any one of claims 1-49, wherein X₂X₃ is NR.

51. The modified AAV capsid protein of any one of claims 1-50, wherein X₁X₂X₃ is ENR.

52. The modified AAV capsid protein of any one of claims 1-50, wherein X₁X₂X₃ is SNR.

53. The modified AAV capsid protein of any one of claims 1-36, wherein X₁X₂X₃ is selected from the group consisting of: DII, DWM, EEI, DML, DWI, SLE, EIN, NHE, DFI, EEL, TEQ, TDA, EDT, NEV, TDW, QFE, EDY, DTT, EPL, SEN, SEQ, TAE, EVN, ELN, DVQ, ETI, EVI, ESV, ETW, SEW, DNW, EVF, EAW, EPF, EIY, EIF, EPY, DVI, DMM, DQI, DHL, DTL, DVL, NDL, DLL, DMQ, NEF, DFL, DIM, TEW, DYI, SDY, DYY, DHF, DKE, DTW, DTI, ELY, TEY, TEI, DAI, DQY, DMY, EWG, DMV, DMI, EPH, QEG, DIN, NEI, EYY, DIV, SEG, DVG, DYQ, EGF, NDI, EGY, DVF, DVH, DGF, DIY, DSF, DGW, EHY, DRE, TEH, DTS, NEN, NEM, NEH, TEN, DSN, DVT, DQS, DKD, DTH, DVV, DQK, NET, DKP, TEV, NDS, QET, EVL, SET, SDT, AEQ, QEF, SEY, SEF, SPF, EGQ, ETH, TDQ, QEA, QDQ, AEN, ESS, NDT, EFM, EFI, EHM, DFM, QDT, SME, DYT, EHV, ENV, EAV, EAI, ESI, DAT, ENQ, EAM, ADN, EFQ, SDS, TDH, SDH, DAS, TWE, SSF, DRD, EFL, TDF, QDA, EMH, SGE, AEW, DAH, TET, TDM, TNE, SAE, NSE, SFE, QDI, DSA, ADV, SEI, AEI, QDV, ADT, DNM, DNQ, ADL, TDL, SDL, SDM, TDV, DNI, DHY, DTY, DAA, DSY, QLY, DVM, DAY, DMT, DQT, DAQ, DTV, DSH, QDF, DST, DNL, DSI, DFV, DNY, DAF, DKI, DKF, DTM, DSL, NDV, TDI, DSV, DAV, DKV, DAM, DNV, DKM, DKL, DKW, DSM, ENI, SDI, DKT, QGE, NNE, QYW, AGM, ESH, QDH, QEH, DYH, ENS, DKQ, TGE, DSS, EST, DSQ, DNS, EFV, SWE, DKN, DKS, SEH, EAL, EAS, ADQ, SDQ, ADI, SDV, EAH, ASE, EAF, EYV, DKA, SNE, AGE, EGV, EQF, ETF, EVY, EQY, QEY, QEN, SAD, AEF, EMF, SDF, ADF, ADY, AVF, TLY, SIY, SLY, AIY, DYV, AEY, ENF, EMY, QDY, ETY, EAY, TVY, SVY, QVF, QVY, TMY, DKY, ALY, AVY, TMW, EQW, SPW, SIW, DTQ, TFF, AMF, EYT, DRV, SPY, SIF, SLF, ENY, EWY, EYI, EFY, TLF, TVF, TYY, TFY, SVF, SYW, TAW, SAW, TIY, NIM, NFM, NII, NIV, SII, SIV, SEM, TTI, NSY, SSY, SNY, SEV, AEH, ALT, SGQ, QTY, SYY, AMY, QMY, SGF, SGY, ALF, TTF, SFN, QAF, ASF, SAF, TAF, TAY, TGL, SAY, QAY, ADH, ASI, SSV, SSI, TGV, SGV, DPH, TEG, DLY, ESE, TEF, NPE, DHA, DLI, DWG, EWM, EET, EIL, TEL, DYL, EMW, EVW, EPV, EDV, SLD, EGH, QRY, TDG, SQE, ELH, EYW, EMM, EFF, QEI, EQG, DVS, ESP, EIQ, EPM, EPQ, SPE, EPN, NQD, ELQ, TDT, ELT, EPI, ETM, ETG, EHI, EMI, EIT, ETV, EVV, EVM, TPY, QEW, EIW, EPT, EIH, DFY, DQL, NDA, DIW, TDY, ELF, DVY, ESG, EPG, EIG, ELG, EVG, DHI, EIS, DYM, DFT, DFH, EQS, TPF, EEF, DWN, SDN, EYL, AET, DMN, QSP, DNA, DHM, DHT, EQI, ESN, EYF, EHF, NAD, AEG, AES, TEM, SEL, DYS, EVH, EAN, EGL, TFE, ETL, TYE, SHE, ELM, TYD, SYE, ESA, EFT, EAT, AMD, AND, DMS, QMD, SLQ, EYS, QDN, EYQ, EKD, QYD, QFD, DER, AFD, EAA, EFS, DHV, QSE, TSD, SFD, DGY, TWD, QWD, EHL, QGD, QSD, AWD, AGD, QIY, EMQ, EAG, ERD, DDR, DAL, QDL, DNT, EGN, ENH, NGE, DYN, DNH, DNF, ESQ, ESM, ESL, SSD, ENL, EMV, QDS, TSE, SWD, ASD, ENT, SSE, EGM, EGS, EGT, EGI, AIF, NIN, NIQ, NYW, EPW, EGG, TIW, QIW, TTW, EKW, NLY, SVM, TPT, AAW, AIH, TIH, QGT, QFF, QYF, QFY, EHH, QLH, TYF, STY, DRL, EYM, SFF, QLF, QIF, APF, TYW, QII, QPF, SPI, TIL, SVL, NVI, SIM, SLL, QEL, AEM, AEL, QEV, QDM, NDM, SYL, ASL, TPL, ANI, NMI, TIM, AEV, TMI, SWI, SFI, SNW, TNF, NAF, ASP, QSQ, APY, SFM, TAN, QAW, SGS, SGH, TGF, QSN, ERY, QMN, TGQ, NGM, NGV, AMN, ALN, QFV, AAF, TEV, TSY, EDK, ASN, SAV, QSV, QGV, QST, ASM, TAI, QSM, QSL, QSI, SSL, AGV, TGT, AGI, QGM, TGM, SGM, SGI, THD, QVE, ALE, QLE, EWP, AFP, AEE, ADE, NIE, TYP, QIE, TFP, SYP, ELW, DVW, NEW, EPA, EVA, TLE, AWE, NME, QME, ATE, AYE, DQM, TPD, DFG, QPE, EDI, EWL, ELL, QPD, EWF, ALD, SVD, QVD, ADA, APD, AHE, EQL, DTA, AFE, DAW, QAE, TME, STE, THE, QID, AAE, EIA, EML, SDA, SIE, TQE, TVD, NWE, NOE, EYG, QYP, EIV, EII, QFP, TID, EQN, EVS, TYG, EAQ, ELV, QLD, TLD, QQD, TIN, TLN, SPN, TPN, NFD, NEY, DSW, NMD, DGA, DYA, DIH, DNP, DQW, DQV, EFA, EPS, DEN, DWV, ELI, DTF, EQT, DHW, ESF, DEK, DMH, EWS, EHA, TTD, EWI, QLQ, SQD, QHD, NSD, EMA, TND, ATD, DGS, TAD, TMD, QES, AHD, QAD, DGH, DGL, NTE, ENG, SMD, NDQ, EMS, ENA, EMT, EYN, DDK, EWA, EWQ, EFH, QWE, DGT, DGM, EWV, EWH, EWT, QND, DGV, ESY, AAD, SND, ANE, TIG, QIG, AVD, SFP, AWP, QDW, SLW, TPA, AII, TIV, SWP, AFY, APN, AVN, TVN, TIS, TIQ, TLQ, AIQ, QVN, NFY, QFG, EMN, NVF, SYF, SFW, TPQ, AIL, TWI, QWN, TWN, QWQ, AQF, ASA, TWV, EWN, TMF, NGI, ATN, SAS, TMQ, TMN, AYN, TQF, SMF, QMQ, SMQ, QAN, AAY, SFQ, TYQ, TFQ, TWQ, AVI, and SGL.

54. The modified AAV capsid protein of any one of claims 1-36, wherein X₁X₂X₃ is selected from the group consisting of: APW, TEL, TDA, QPY, SPN, EHY, DWK, DLK, DFK, DVK, NSI, DIR, SPF, SEL, DRT, DRF, ADL, TDL, SDL, DNY, DKI, NDV, DKM, DNH, DNF, DSS, EST, EWT, DKN, DKS, SEH, ESQ, ESL, QND, EAH, AIF, AVF, QVF, TMY, ALY, NNG, NIF, NTF, NFF, AWF, NPY, SWF, AII, AYF, AQW, NFY, AGP, QQF, TKE, TNG, NSF, NAW, QAG, ERG, NKD, QSG, QNG, EAK, QWF, SWY, TFF, TYF, NYY, QFG, NWA, AMF, STY, TNW, ANW, AWM, TSF, DRL, DRV, SPY, NVF, SIF, QLF, SLF, QIF, SYF, APF, AGF, SVF, SAW, TIY, DHK, DAK, EGK, DYK, QNL, QPL, SPV, NPM, STL, NIL, AGA, NTV, SQA, QQA, NNF, NWL, NMA, NNA, NHN, NFH, ELR, NYM, ERA, NFS, SRD, NMM, NNL, NNQ, NNS, QDR, SDK, NNI, AQH, ANA, QNH, QWG, AWA, QWA, EQK, SHS, QFA, SFA, TFA, AYG, EKQ, ERN, EMR, EFR, AYM, EAR, EMK, EYK, EWK, QPI, QPF, NPL, SPI, SPL, NPI, SIV, SLL, SSA, AEM, TML, TLL, SML, TYL, QDM, NSN, AQL, NAT, SQQ, QTI, NAH, SQL, STH, NTT, AEK, ANV, QGL, AGL, QMV, STM, NOM, STI, TQL, SMM, SKE, SQT, SYL, ASL, SLV, NAM, TTV, TPI, TPL, ATL, DNK, DSR, ANL, SHL, SNL, TNL, QNI, QAL, ANI, QNM, QYM, SQI, SNI, QNV, SHV, SHM, QMM, ANM, AHM, SQM, NAL, AQT, EKL, NNM, TNM, SNM, QQI, TQV, TQI, THM, SQV, AAV, THL, AQI, NNV, TNN, TNI, SNN, TQM, NAI, TNV, SWT, NMV, NQI, NQL, NMI, NSS, SHF, QNF, SWL, AQM, SYV, TFM, SYM, TYM, ATI, TTI, SLI, ALI, AYI, QLV, TMI, SMI, QFI, NMY, NAY, NMN, NMF, ANF, NWT, QWM, TFT, SWM, TWM, QNW, STF, AFM, NSY, SSY, SNY, THY, TNY, QNY, THE, NSV, ATY, SKD, TNF, TWT, QWT, SFS, TFS, DRQ, NYN, DFR, EKG, ENN, AWN, QWQ, TQH, ALV, QHQ, DVR, STS, SQS, AAA, QGQ, QSQ, QAQ, SIH, APY, QGN, AGQ, ASQ, AAQ, SQN, NON, ERM, ALH, NYH, TGH, ATH, QTH, TQT, QQH, AQF, QMH, EKN, ERV, ERS, AFT, EKF, EKT, QSA, NFT, QYH, NYS, TSA, QYY, AYY, AHY, QFN, AWQ, NWQ, QAW, NMS, NWN, QMF, AMH, TSN, TAH, TMH, QFH, NWS, SWN, QRD, QKD, AKE, TSS, AYH, TSH, AHQ, ADK, ADR, ASA, ASS, SNS, QSS, SAH, SMH, QAH, AAH, AHN, QNS, QAS, ANS, ANN, SWA, SWS, TWS, ERI, EKS, QWS, AYS, AFS, SYS, QFS, QYN, QFT, AYT, TNS, TYS, SFT, QNQ, QYS, SYT, ANQ, SWV, SNQ, ANY, TNQ, ATF, SSQ, SSN, TWA, SGQ, SSS, TWY, SMY, TGN, TGY, QWY, SQF, TGF, QTY, TTY, AMY, QMY, ERY, TGQ, SGY, TMF, ALN, ARD, ATN, NAS, SAS, AQN, SYG, SYN, TFN, TMN, AYN, SGN, QNN, DKG, AAN, SMN, SAN, TTF, TQF, SFN, QAF, QFV, AAF, ASF, SAF, SMF, ASY, AFN, TAF, TSY, TAY, QMQ, SMQ, TYV, TGL, QAY, AAY, ADH, EDK, ASN, QYQ, SFQ, TYQ, TWQ, SWQ, AYQ, SYQ, AMV, TMV, TAV, AQV, TYT, TAT, QGI, QQV, SAQ, NAV, SAV, ASV, QSV, QAV, QAI, QAM, TAM, DTR, DOR, DMR, NNT, NYT, NMT, NST, ATT, SMV, SNT, QNT, AAT, SAM, SAL, QGV, QST, QYT, ASM, TSM, AMT, TST, ANH, SNV, TNT, ANT, SST, AST, TAI, DAR, SSM, TAL, NSM, TSL, SNF, TSI, TSV, TDK, SAT, SAI, QSL, QSI, ASI, SSL, SSV, SSI, AGI, QGM, TGM, SGM, SGL, SGV, ARL, NWE, SFL, TPY, DRP, DTK, DMK, SEY, SME, EAI, AHD, ADN, EFQ, EMH, SGE, DNQ, DNI, QLY, DGT, EGN, DYN, EWV, ESM, EAL, ASD, DKA, ADF, ADY, SLY, AEY, ETY, EAY, TGA, AVM, TPT, SVI, QSH, TTQ, QTN, TLH, AWY, SLT, AWV, QQY, NFV, SFY, TYY, SHA, TEK, QFK, NNY, EPR, NIT, NHI, NTY, SHY, NEM, NEN, NDK, NER, SER, NDR, QDK, SDR, QNA, TNA, THT, SNH, AYA, NFA, AFA, AHA, QHA, EYR, ENR, EHK, QVL, SVL, NIV, NVI, ADM, SAA, SQH, NQV, ALL, ATM, QYA, STT, DGR, NHT, SHT, QHT, TTL, NHM, QHL, DNR, QHM, QFM, QMI, AHV, THI, AHT, TNH, NHV, THV, SHI, ATV, TWI, NYV, TYI, DYR, SFI, NQF, NWM, NSL, NSP, TQY, DRS, ASP, TKD, QWI, QWN, TWN, QWV, THN, TTM, TQQ, NMQ, TYA, STQ, STN, AGN, EKH, ERT, TFH, SFH, AFH, ENK, NYQ, SFM, QGS, AHF, QLT, QHY, AQY, TRD, ERH, QWH, TYH, TRE, TAQ, AER, TDR, ASH, EKI, AFV, TYN, ALT, NGN, SYY, EWN, NGM, NAN, TAS, TFV, SFV, QAN, SGT, SAY, AFQ, TFQ, QFQ, EFK, SYH, QHV, AMM, THQ, AAI, AAM, NSQ, QAT, TSQ, SHQ, QSM, AGV, AGT, TGT, TGI, AVI, TGV, SGI, ESP, SDA, TPN, EAW, NEQ, DSN, DIK, DNA, QES, SLQ, EKD, SDH, SSF, TET, QDI, DSA, DNM, DKT, DGM, SQY, TLT, QEH, DKQ, TGE, DNS, SDV, SNE, EGT, EGV, AAP, AHG, TWK, TQA, TVT, APQ, SVT, NGT, SSH, NLV, AGY, QFF, QYF, NWV, EKA, EKY, SMG, NAG, ASG, TFI, ELK, SEK, QTL, NML, DHS, ETR, NQQ, QWL, ANP, AHH, NEK, NNN, SNA, EWR, ESR, SHN, SFG, SYA, APV, QPV, SPT, TPQ, QVI, TPV, AVL, SEM, NDM, API, QLM, QTM, QQL, QHI, NFL, AYV, NKE, TIM, QYV, SWI, DRN, AWI, SMA, NRE, QTS, QVV, QQT, QMA, QQS, QAA, ATQ, ERL, TGS, QQQ, AQQ, QHF, TAN, QSY, QSN, NGQ, TWH, TMS, QMS, TON, TMT, QTV, THS, TTT, SMT, QMT, SMR, ADA, AAE, EPY, EYL, NWI, TSP, DGQ, NDH, QFD, EHL, ERD, DAF, ENI, ENH, QQW, AGM, ENL, EAF, EYV, ENT, SSE, AGE, QEY, DRH, NIQ, SVV, SVM, QPT, TVL, QTQ, SVQ, TIQ, NLQ, SIQ, AWL, EHH, NGY, NHF, NSA, NAA, NTH, QLI, NWH, QRE, AML, EVR, SNP, NHS, THH, NQS, TTS, AHS, EKV, NFQ, APL, QPM, NPV, SIL, TII, STV, QEL, TFL, SIT, NAQ, TMM, AAL, QQM, AMI, QFL, AHI, SYI, SSP, NVV, TTN, TAA, TOS, TMA, ALA, QQN, NSH, TTH, SRE, ERQ, QHS, SWH, SEV, AVT, ALQ, DKH, AMQ, ARE, TWV, NGL, NGV, TMQ, TLV, EER and QTT.

55. The modified AAV capsid protein of any one of claims 1-36, wherein X₁X₂X₃ is selected from the group consisting of: DMK, ATD, EEK, QMD, EFS, ERD, DDR, TDM, SAE, EHS, ENH, SWE, SNE, NNG, QAG, ERG, QSG, QNG, ASG, QFG, AMF, ELR, NFM, NNS, NNI, SDR, EQR, EHR, EWR, EQK, ESR, EKQ, EYR, ENR, EMK, EYK, EHK, EWK, QNI, TNI, TYI, SNY, DRQ, AWI, QWI, DER, EKG, QYG, QWQ, EKN, EKF, EKT, AFH, ENK, NYS, DKH, AAG, QMF, QFH, QKD, ARE, AHQ, ADK, ADR, AHN, QNS, ANN, SWS, EKS, AFS, QFS, TNQ, TGY, NGL, ARD, AKD, AAF, SMF, AFN, SGT, TGL, ASN, SFQ, AFQ, TFQ, QFQ, EFK, SWQ, AYQ, SYQ, DQR, DMR, ASM, ANT, SHQ, TSL, SNF, QSL, ASI, SGL, SGI, NDA, DHQ, DFK, DTK, DNA, SDS, TDH, DGL, QDV, SDM, DKT, DGM, SQY, DKS, SSD, EMV, TSE, EYV, SSE, EGM, SDF, TTG, QLG, NNP, AGS, QSF, TLM, NGA, AWY, NKD, SWY, TFF, NSG, NGY, QEK, SEK, TEK, AQA, NVL, NHI, TER, NML, NMA, NNA, NHN, ANP, AMS, NER, SER, DKK, NMM, QDK, AHS, TNA, THT, SNH, QNH, ESK, SHS, AYM, SSA, NAT, QLM, QQM, SQI, SNI, QNV, SHV, SHM, NNV, TNN, AHT, TNH, NHV, TQM, THV, SHI, NYL, NMV, NFL, AYV, NQI, NOL, NMI, NSS, SHF, SYM, NYV, AYI, SWI, QFI, ANF, TFT, TWM, QNW, NSY, THY, TNY, NYN, AWN, SMA, NSH, TGH, SFH, SWH, SFM, NFT, QYH, QGS, TGS, TSA, QQQ, QYY, AMQ, TAN, AWH, NMS, TSN, TAH, TMH, QWH, NWS, SWN, QRD, TYH, AKE, TAQ, AER, AYH, ASA, SNS, QSS, SAH, SMH, ASH, QAS, ANS, SYS, QYN, TYN, TNS, SFT, ANQ, SGS, SGH, SSS, TWY, QTY, SYY, NGM, TMS, SYG, TON, SYN, TFN, TMN, AYN, SGN, QNN, SMN, SAN, QAF, ASF, ASY, TAS, TAF, TFV, SMQ, SFV, TMT, QAN, SAY, EDK, AMV, QHV, THS, TAT, QGI, QQV, NAV, SAV, ASV, QSV, QAV, AAI, AAM, QAM, TAM, DTR, NNT, NYT, NMT, ATT, QTT, TTT, QAT, SMV, SNT, QNT, AAT, SAM, SAL, QGV, QST, QYT, TSM, ANH, SNV, TSQ, TNT, SST, AST, TAI, TSI, TSV, TDK, SAT, QSM, SAI, QSI, SSV, SSI, AGT, TGT, AGI, QGM, TGM, TGV, SGM, SGV, SQE, EHT, EYL, DSG, SFE, QND, TQA, AWV, NFV, TSG, SSG, DRG, ANW, TFI, TSF, QNL, STL, TWL, NNF, NND, NYM, NFI, ETR, SNP, EIR, NFS, NNH, NNL, AHL, NNQ, NDR, NNN, QDR, SDK, SNA, AFG, NYA, QHA, QFA, SHN, NFQ, SYA, TFA, EMR, EFR, EAR, NQM, TTL, TQL, SMM, SKE, SYL, ASL, SLV, TTV, AAL, NHL, NHM, QYL, QHL, ATL, DNR, DSR, ANL, SHL, SNL, TNL, QAL, AMI, ANI, QNM, QYM, QHM, QFM, QMM, QMI, ANM, AHM, SQM, AQT, EKL, NNM, TNM, SNM, QQI, TQV, TQI, THI, QHI, THL, SNN, TNV, SWL, AQM, SYV, TFM, TYM, TWI, AFI, ATI, DYR, QLV, TMI, SYI, SFI, NWM, NWT, NSL, STF, SNW, AFM, SSY, NSV, TNF, NAF, TFS, QWV, ERT, TQT, AFT, NYQ, ERL, NRD, TRD, AWQ, TDR, TSH, ASS, AYT, TYS, QYS, SYT, SWV, SNQ, SMS, NAS, SAS, QFV, SAF, TWQ, TAV, AMM, TYT, QAI, TST, DAR, SSM, TAL, NES, DWK, DLR, DIR, SDT, EAN, ETH, TYD, SYE, EAM, TND, AYD, TSP, DRT, NDH, SSF, EFH, DNQ, DSH, DNT, SDI, DKQ, EWT, EAL, EAS, ESL, ASD, EAH, ASE, EAF, ENT, SIY, QDY, APG, AIG, SSW, NPG, ERP, TVI, QSH, SLS, EDR, AWL, SLH, EQH, EHH, NWV, EKY, STY, DRI, EYH, DRV, SPY, NNY, DWR, ERA, QRE, EVR, SRD, AWA, SFA, QTI, SHT, THM, AQI, AHI, SWT, ATV, TTI, SMI, QYV, NMY, SWM, QNY, TWT, QWT, TQY, NRE, QTS, QHQ, DVR, AAA, TYA, EKH, ALH, AWT, ATH, SRE, TFH, QMH, ERV, ERS, QHY, ALQ, QSY, ERH, TRE, TSS, QAH, AAH, SWA, TWS, QWS, AYS, AFV, QNQ, ANY, ALT, SMY, TWV, TTY, AMY, QMY, ERY, TWH, TSY, TAY, TYV, QAY, AAY, QYQ, TYQ, SYH, AQV, EER, THQ, and SMT.

56. The modified AAV capsid protein of any one of claims 1-55, wherein RGDX₇X₈X₉X₁₀ has an amino acid sequence selected from SEQ ID NOs.: 238-44858.

57. The modified AAV capsid protein of any one of claims 1-55, wherein RGDX₇X₈X₉X₁₀ has an amino acid sequence selected from SEQ ID NOs.: 238-248.

58. The modified AAV capsid protein of any one of claims 1-55 wherein RGDX₇X₈X₉X₁₀ has an amino acid sequence selected from SEQ ID NOs.: 238-338.

59. The modified AAV capsid protein of any one of claims 1-55, wherein RGDX₇X₈X₉X₁₀ has an amino acid sequence selected from SEQ ID NOs.: 238-438.

60. The modified AAV capsid protein of any one of claims 1-55, wherein RGDX₇X₈X₉X₁₀ has an amino acid sequence selected from SEQ ID NOs.: 238-538.

61. The modified AAV capsid protein of any one of claims 1-55, wherein RGDX₇X₈X₉X₁₀ has an amino acid sequence selected from SEQ ID NOs.: 238-638.

62. The modified AAV capsid protein of any one of claims 1-55, wherein RGDX₇X₈X₉X₁₀ has an amino acid sequence selected from SEQ ID NOs.: 238-738.

63. The modified AAV capsid protein of any one of claims 1-55, wherein RGDX₇X₈X₉X₁₀ has an amino acid sequence selected from SEQ ID NOs.: 3881, 12092, 14601, 15342, 21498, and 31396.

64. The modified AAV capsid protein of any one of claims 1-55, wherein RGDX₇X₈X₉X₁₀ has the amino acid sequence of SEQ ID NO: 238.

65. The modified AAV capsid protein of any one of claims 1-55, wherein X₁X₂X₃RGDX₇X₈X₉X₁₀ has an amino acid sequence selected from SEQ ID NOs.: 44859-44883, 44911, 44912, 44913, 44918-44919, and 48391-157057.

66. The modified AAV capsid protein of any one of claims 1-55, wherein X₁X₂X₃RGDX₇X₈X₉X₁₀ has an amino acid sequence selected from SEQ ID NOs.: 44864-44867, and 44879-44883.

67. The modified AAV capsid protein of any one of claims 1-55, wherein X₁X₂X₃RGDX₇X₈X₉X₁₀ has an amino acid sequence selected from SEQ ID NOs.: 44859-44878, 44911, 44912, 44913, 44918-44919, and 48391-157057.

68. The modified AAV capsid protein of any one of claims 1-55, wherein the X₇ is selected from R, F, H, L, Q, R, and Y.

69. The modified AAV capsid protein of claim 68, wherein the X₇ is R.

70. The modified AAV capsid protein of claim 68, wherein the X₇ is Y or H.

71. The modified AAV capsid protein of claim 68, wherein the X₇ is F or Y.

72. The modified AAV capsid protein of any one of claims 1-71, wherein X₈ is selected from S, G, D, I, L, N, Q, T, and V.

73. The modified AAV capsid protein of claim 72, wherein X₈ is T, G or S.

74. The modified AAV capsid protein of claim 72, wherein X₈ is N or Q.

75. The modified AAV capsid protein of claim 72, wherein X₈ is S or G.

76. The modified AAV capsid protein of claims 1-75, wherein X₉ is selected from V, S, N, G, Q, L, T, and Y.

77. The modified AAV capsid protein of claim 76, wherein X₉ is selected from V or Q.

78. The modified AAV capsid protein of claim 76, wherein X₉ is selected from S or V.

79. The modified AAV capsid protein of claim 76, wherein X₉ is selected from N or S.

80. The modified AAV capsid protein of any one of claims 1-79, wherein X₁₀ is selected from I, V, S, L, M, R, T, and Q.

81. The modified AAV capsid protein of claim 80, wherein X₁₀ is I or V.

82. The modified AAV capsid protein of claim 80, wherein X₁₀ is V, L or M.

83. The modified AAV capsid protein of claim 80, wherein X₁₀ is T or L.

84. The modified AAV capsid protein of claim 1-55, wherein the targeting peptide has a sequence of X₁X₂X₃RGDRGVV (SEQ ID NO: 98928), X₁X₂X₃RGDRSVV (SEQ ID NO: 98931), X₁X₂X₃RGDRGQI (SEQ ID NO: 98927), X₁X₂X₃RGDRSQT (SEQ ID NO: 98930), X₁X₂X₃RGDRQGI (SEQ ID NO: 98929), X₁X₂X₃RGDFQNT (SEQ ID NO: 98934), X₁X₂X₃RGDHGVL (SEQ ID NO: 98938), X₁X₂X₃RGDYTSV (SEQ ID NO: 98941), X₁X₂X₃RGDYTSM (SEQ ID NO: 98942), X₁X₂X₃RGDLTVT (SEQ ID NO: 98935), X₁X₂X₃RGDFNNT (SEQ ID NO: 98943), X₁X₂X₃RGDYSSV (SEQ ID NO: 98937), X₁X₂X₃RGDHVNL (SEQ ID NO: 98924), X₁X₂X₃RGDQSTL (SEQ ID NO: 98926), X₁X₂X₃RGDLIGR (SEQ ID NO: 98925), X₁X₂X₃RGDENNL (SEQ ID NO: 98933), X₁X₂X₃RGDLLLS (SEQ ID NO: 98932), X₁X₂X₃RGDYNSL (SEQ ID NO: 98940), X₁X₂X₃RGDRDYL (SEQ ID NO: 98939), or X₁X₂X₃RGDYVGL (SEQ ID NO: 98936).

85. The modified AAV capsid protein of claim 84, wherein the targeting peptide has a sequence of X₁X₂X₃RGDRGVV (SEQ ID NO: 98928), X₁X₂X₃RGDRSVV (SEQ ID NO: 98931) or X₁X₂X₃RGDRGQI (SEQ ID NO: 98927).

86. The modified AAV capsid protein of claim 84, wherein the targeting peptide has a sequence of X₁X₂X₃RGDYTSV (SEQ ID NO: 98941), X₁X₂X₃RGDYTSM (SEQ ID NO: 98942), X₁X₂X₃RGDRGVV (SEQ ID NO: 98928), X₁X₂X₃RGDRSVV (SEQ ID NO: 98931), X₁X₂X₃RGDYSSV (SEQ ID NO: 98937), or X₁X₂X₃RGDHGVL (SEQ ID NO: 98938).

87. The modified AAV capsid protein of any one of claims 1-55, wherein the targeting peptide has a sequence of X₁X₂X₃RGDFQNT (SEQ ID NO: 98934), X₁X₂X₃RGDHGVL (SEQ ID NO: 98938), X₁X₂X₃RGDLIGR (SEQ ID NO: 98925), X₁X₂X₃RGDRGQI (SEQ ID NO: 98927), X₁X₂X₃RGDRGVV (SEQ ID NO: 98928), X₁X₂X₃RGDYTSM (SEQ ID NO: 98942) or X₁X₂X₃RGDYTSV (SEQ ID NO: 98941).

88. The modified AAV capsid protein of claim 87, wherein X₁X₂X₃ is selected from the group consisting of EFK, AAY, DQK, QVY, DKL, DNV, ENF, EWK, QNV, and TFM.

89. The modified AAV capsid protein of any one of claims 1-55 wherein the targeting peptide has an amino acid sequence selected from: RGDRSX₉I, RGDRGX₉I, RGDRSX₉V, or RGDRGX₉V.

90. The modified AAV capsid protein of claim 89, wherein the targeting peptide has an amino acid sequence selected from X₁X₂X₃RGDRGQI (SEQ ID NO: 98927), X₁X₂X₃RGDRSVV (SEQ ID NO: 98931) or X₁X₂X₃RGDRGVV (SEQ ID NO: 98928).

91. The modified AAV capsid protein of any one of claims 89-90, wherein X₁ is D or E, X₂ is K, E, D, A, S, F or N, and X₃ is Y, V, or F.

92. The modified AAV capsid protein of any one of claims 89-91, wherein X₁X₂X₃ is selected from the group consisting of: DII, DWM, EEI, DML, DWI, SLE, EIN, NHE, DFI, EEL, TEQ, TDA, EDT, NEV, TDW, QFE, EDY, DTT, EPL, SEN, SEQ, TAE, EVN, ELN, DVQ, ETI, EVI, ESV, ETW, SEW, DNW, EVF, EAW, EPF, EIY, EIF, EPY, DVI, DMM, DQI, DHL, DTL, DVL, NDL, DLL, DMQ, NEF, DFL, DIM, TEW, DYI, SDY, DYY, DHF, DKE, DTW, DTI, ELY, TEY, TEI, DAI, DQY, DMY, EWG, DMV, DMI, EPH, QEG, DIN, NEI, EYY, DIV, SEG, DVG, DYQ, EGF, NDI, EGY, DVF, DVH, DGF, DIY, DSF, DGW, EHY, DRE, TEH, DTS, NEN, NEM, NEH, TEN, DSN, DVT, DQS, DKD, DTH, DVV, DQK, NET, DKP, TEV, NDS, QET, EVL, SET, SDT, AEQ, QEF, SEY, SEF, SPF, EGQ, ETH, TDQ, QEA, QDQ, AEN, ESS, NDT, EFM, EFI, EHM, DFM, QDT, SME, DYT, EHV, ENV, EAV, EAI, ESI, DAT, ENQ, EAM, ADN, EFQ, SDS, TDH, SDH, DAS, TWE, SSF, DRD, EFL, TDF, QDA, EMH, SGE, AEW, DAH, TET, TDM, TNE, SAE, NSE, SFE, QDI, DSA, ADV, SEI, AEI, QDV, ADT, DNM, DNQ, ADL, TDL, SDL, SDM, TDV, DNI, DHY, DTY, DAA, DSY, QLY, DVM, DAY, DMT, DQT, DAQ, DTV, DSH, QDF, DST, DNL, DSI, DFV, DNY, DAF, DKI, DKF, DTM, DSL, NDV, TDI, DSV, DAV, DKV, DAM, DNV, DKM, DKL, DKW, DSM, ENI, SDI, DKT, QGE, NNE, QYW, AGM, ESH, QDH, QEH, DYH, ENS, DKQ, TGE, DSS, EST, DSQ, DNS, EFV, SWE, DKN, DKS, SEH, EAL, EAS, ADQ, SDQ, ADI, SDV, EAH, ASE, EAF, EYV, DKA, SNE, AGE, EGV, EQF, ETF, EVY, EQY, QEY, QEN, SAD, AEF, EMF, SDF, ADF, ADY, AVF, TLY, SIY, SLY, AIY, DYV, AEY, ENF, EMY, QDY, ETY, EAY, TVY, SVY, QVF, QVY, TMY, DKY, ALY, AVY, TMW, EQW, SPW, SIW, DTQ, TFF, AMF, EYT, DRV, SPY, SIF, SLF, ENY, EWY, EYI, EFY, TLF, TVF, TYY, TFY, SVF, SYW, TAW, SAW, TIY, NIM, NFM, NII, NIV, SII, SIV, SEM, TTI, NSY, SSY, SNY, SEV, AEH, ALT, SGQ, QTY, SYY, AMY, QMY, SGF, SGY, ALF, TTF, SFN, QAF, ASF, SAF, TAF, TAY, TGL, SAY, QAY, ADH, ASI, SSV, SSI, TGV, SGV, DPH, TEG, DLY, ESE, TEF, NPE, DHA, DLI, DWG, EWM, EET, EIL, TEL, DYL, EMW, EVW, EPV, EDV, SLD, EGH, QRY, TDG, SQE, ELH, EYW, EMM, EFF, QEI, EQG, DVS, ESP, EIQ, EPM, EPQ, SPE, EPN, NQD, ELQ, TDT, ELT, EPI, ETM, ETG, EHI, EMI, EIT, ETV, EVV, EVM, TPY, QEW, EIW, EPT, EIH, DFY, DQL, NDA, DIW, TDY, ELF, DVY, ESG, EPG, EIG, ELG, EVG, DHI, EIS, DYM, DFT, DFH, EQS, TPF, EEF, DWN, SDN, EYL, AET, DMN, QSP, DNA, DHM, DHT, EQI, ESN, EYF, EHF, NAD, AEG, AES, TEM, SEL, DYS, EVH, EAN, EGL, TFE, ETL, TYE, SHE, ELM, TYD, SYE, ESA, EFT, EAT, AMD, AND, DMS, QMD, SLQ, EYS, QDN, EYQ, EKD, QYD, QFD, DER, AFD, EAA, EFS, DHV, QSE, TSD, SFD, DGY, TWD, QWD, EHL, QGD, QSD, AWD, AGD, QIY, EMQ, EAG, ERD, DDR, DAL, QDL, DNT, EGN, ENH, NGE, DYN, DNH, DNF, ESQ, ESM, ESL, SSD, ENL, EMV, QDS, TSE, SWD, ASD, ENT, SSE, EGM, EGS, EGT, EGI, AIF, NIN, NIQ, NYW, EPW, EGG, TIW, QIW, TTW, EKW, NLY, SVM, TPT, AAW, AIH, TIH, QGT, QFF, QYF, QFY, EHH, QLH, TYF, STY, DRL, EYM, SFF, QLF, QIF, APF, TYW, QII, QPF, SPI, TIL, SVL, NVI, SIM, SLL, QEL, AEM, AEL, QEV, QDM, NDM, SYL, ASL, TPL, ANI, NMI, TIM, AEV, TMI, SWI, SFI, SNW, TNF, NAF, ASP, QSQ, APY, SFM, TAN, QAW, SGS, SGH, TGF, QSN, ERY, QMN, TGQ, NGM, NGV, AMN, ALN, QFV, AAF, TFV, TSY, EDK, ASN, SAV, QSV, QGV, QST, ASM, TAI, QSM, QSL, QSI, SSL, AGV, TGT, AGI, QGM, TGM, SGM, SGI, THD, QVE, ALE, QLE, EWP, AFP, AEE, ADE, NIE, TYP, QIE, TFP, SYP, ELW, DVW, NEW, EPA, EVA, TLE, AWE, NME, QME, ATE, AYE, DQM, TPD, DFG, QPE, EDI, EWL, ELL, QPD, EWF, ALD, SVD, QVD, ADA, APD, AHE, EQL, DTA, AFE, DAW, QAE, TME, STE, THE, QID, AAE, EIA, EML, SDA, SIE, TQE, TVD, NWE, NOE, EYG, QYP, EIV, EII, QFP, TID, EQN, EVS, TYG, EAQ, ELV, QLD, TLD, QQD, TIN, TLN, SPN, TPN, NFD, NEY, DSW, NMD, DGA, DYA, DIH, DNP, DQW, DQV, EFA, EPS, DFN, DWV, ELI, DTF, EQT, DHW, ESF, DEK, DMH, EWS, EHA, TTD, EWI, QLQ, SQD, QHD, NSD, EMA, TND, ATD, DGS, TAD, TMD, QES, AHD, QAD, DGH, DGL, NTE, ENG, SMD, NDQ, EMS, ENA, EMT, EYN, DDK, EWA, EWQ, EFH, QWE, DGT, DGM, EWV, EWH, EWT, QND, DGV, ESY, AAD, SND, ANE, TIG, QIG, AVD, SFP, AWP, QDW, SLW, TPA, AII, TIV, SWP, AFY, APN, AVN, TVN, TIS, TIQ, TLQ, AIQ, QVN, NFY, QFG, EMN, NVF, SYF, SFW, TPQ, AIL, TWI, QWN, TWN, QWQ, AQF, ASA, TWV, EWN, TMF, NGI, ATN, SAS, TMQ, TMN, AYN, TQF, SMF, QMQ, SMQ, QAN, AAY, SFQ, TYQ, TFQ, TWQ, AVI, and SGL.

93. The modified AAV capsid protein of any one of claims 89-92, wherein X₁X₂X₃ is selected from the group consisting of: DAV, DKW, EAY, AEY, DFV, DKF, DKI, DKL, DNV, DNY, DSL, DSV, EFI, SEF, SEY, SLY, ADF, ADY, ALY, AVF, DAF, DAL, DAM, DAT, DHV, DIV, DKA, DKM, DKT, DKV, DKY, DMI, DNF, DNI, DQT, DSI, DVY, DYN, DYV, EAT, EAW, EFV, EGL, EIY, EMF, EMY, ENF, EPF, EPY, EQY, ESY, ETF, EWI, EWT, EYI, EYV, NEM, QDF, QDY, QEY, QLY, QND, QVF, QVY, SDL, SDV, SEH, SII, SIY, SSL, SVY, SWD, SWE, TDF, TDV, TDY, TEF, TEY, TIY, TLY, and TWQ.

94. The modified AAV capsid protein of any one of claims 1-55, wherein X₇ is selected from Y and H; X₈ is selected from T, G and S; X₉ is selected from S and V; X₁₀ is selected from V, L and M.

95. The modified AAV capsid protein of claim 94, wherein the targeting peptide has an amino acid sequence selected from: X₁X₂X₃RGDHGVL (SEQ ID NO: 98938), X₁X₂X₃RGDYSSV (SEQ ID NO: 98937), X₁X₂X₃RGDYTSM (SEQ ID NO: 98942) or X₁X₂X₃RGDYTSV (SEQ ID NO: 98941).

96. The modified AAV capsid protein of claim 94 or 95, wherein X₁ is S, A or T, X₂ is N, A or Y, and X₃ is I, V, M, Q, T, Y or K.

97. The modified AAV capsid protein of any one of claims 94-96, wherein X₁X₂X₃ is selected from the group consisting of: APW, TEL, TDA, QPY, SPN, EHY, DWK, DLK, DFK, DVK, NSI, DIR, SPF, SEL, DRT, DRF, ADL, TDL, SDL, DNY, DKI, NDV, DKM, DNH, DNF, DSS, EST, EWT, DKN, DKS, SEH, ESQ, ESL, QND, EAH, AIF, AVF, QVF, TMY, ALY, NNG, NIF, NTF, NFF, AWF, NPY, SWF, AII, AYF, AQW, NFY, AGP, QQF, TKE, TNG, NSF, NAW, QAG, ERG, NKD, QSG, QNG, EAK, QWF, SWY, TFF, TYF, NYY, QFG, NWA, AMF, STY, TNW, ANW, AWM, TSF, DRL, DRV, SPY, NVF, SIF, QLF, SLF, QIF, SYF, APF, AGF, SVF, SAW, TIY, DHK, DAK, EGK, DYK, QNL, QPL, SPV, NPM, STL, NIL, AGA, NTV, SQA, QQA, NNF, NWL, NMA, NNA, NHN, NFH, ELR, NYM, ERA, NFS, SRD, NMM, NNL, NNQ, NNS, QDR, SDK, NNI, AQH, ANA, QNH, QWG, AWA, QWA, EQK, SHS, QFA, SFA, TFA, AYG, EKQ, ERN, EMR, EFR, AYM, EAR, EMK, EYK, EWK, QPI, QPF, NPL, SPI, SPL, NPI, SIV, SLL, SSA, AEM, TML, TLL, SML, TYL, QDM, NSN, AQL, NAT, SQQ, QTI, NAH, SQL, STH, NTT, AEK, ANV, QGL, AGL, QMV, STM, NOM, STI, TQL, SMM, SKE, SQT, SYL, ASL, SLV, NAM, TTV, TPI, TPL, ATL, DNK, DSR, ANL, SHL, SNL, TNL, QNI, QAL, ANI, QNM, QYM, SQI, SNI, QNV, SHV, SHM, QMM, ANM, AHM, SQM, NAL, AQT, EKL, NNM, TNM, SNM, QQI, TQV, TQI, THM, SQV, AAV, THL, AQI, NNV, TNN, TNI, SNN, TQM, NAI, TNV, SWT, NMV, NQI, NQL, NMI, NSS, SHE, QNF, SWL, AQM, SYV, TFM, SYM, TYM, ATI, TTI, SLI, ALI, AYI, QLV, TMI, SMI, QFI, NMY, NAY, NMN, NMF, ANF, NWT, QWM, TFT, SWM, TWM, QNW, STF, AFM, NSY, SSY, SNY, THY, TNY, QNY, THE, NSV, ATY, SKD, TNF, TWT, QWT, SFS, TFS, DRQ, NYN, DFR, EKG, ENN, AWN, QWQ, TQH, ALV, QHQ, DVR, STS, SQS, AAA, QGQ, QSQ, QAQ, SIH, APY, QGN, AGQ, ASQ, AAQ, SQN, NON, ERM, ALH, NYH, TGH, ATH, QTH, TQT, QQH, AQF, QMH, EKN, ERV, ERS, AFT, EKF, EKT, QSA, NFT, QYH, NYS, TSA, QYY, AYY, AHY, QFN, AWQ, NWQ, QAW, NMS, NWN, QMF, AMH, TSN, TAH, TMH, QFH, NWS, SWN, QRD, QKD, AKE, TSS, AYH, TSH, AHQ, ADK, ADR, ASA, ASS, SNS, QSS, SAH, SMH, QAH, AAH, AHN, QNS, QAS, ANS, ANN, SWA, SWS, TWS, ERI, EKS, QWS, AYS, AFS, SYS, QFS, QYN, QFT, AYT, TNS, TYS, SFT, QNQ, QYS, SYT, ANQ, SWV, SNQ, ANY, TNQ, ATF, SSQ, SSN, TWA, SGQ, SSS, TWY, SMY, TGN, TGY, QWY, SQF, TGF, QTY, TTY, AMY, QMY, ERY, TGQ, SGY, TMF, ALN, ARD, ATN, NAS, SAS, AQN, SYG, SYN, TFN, TMN, AYN, SGN, QNN, DKG, AAN, SMN, SAN, TTF, TQF, SFN, QAF, QFV, AAF, ASF, SAF, SMF, ASY, AFN, TAF, TSY, TAY, QMQ, SMQ, TYV, TGL, QAY, AAY, ADH, EDK, ASN, QYQ, SFQ, TYQ, TWQ, SWQ, AYQ, SYQ, AMV, TMV, TAV, AQV, TYT, TAT, QGI, QQV, SAQ, NAV, SAV, ASV, QSV, QAV, QAI, QAM, TAM, DTR, DQR, DMR, NNT, NYT, NMT, NST, ATT, SMV, SNT, QNT, AAT, SAM, SAL, QGV, QST, QYT, ASM, TSM, AMT, TST, ANH, SNV, TNT, ANT, SST, AST, TAI, DAR, SSM, TAL, NSM, TSL, SNF, TSI, TSV, TDK, SAT, SAI, QSL, QSI, ASI, SSL, SSV, SSI, AGI, QGM, TGM, SGM, SGL, SGV, ARL, NWE, SFL, TPY, DRP, DTK, DMK, SEY, SME, EAI, AHD, ADN, EFQ, EMH, SGE, DNQ, DNI, QLY, DGT, EGN, DYN, EWV, ESM, EAL, ASD, DKA, ADF, ADY, SLY, AEY, ETY, EAY, TGA, AVM, TPT, SVI, QSH, TTQ, QTN, TLH, AWY, SLT, AWV, QQY, NFV, SFY, TYY, SHA, TEK, QFK, NNY, EPR, NIT, NHI, NTY, SHY, NEM, NFN, NDK, NER, SER, NDR, QDK, SDR, QNA, TNA, THT, SNH, AYA, NFA, AFA, AHA, QHA, EYR, ENR, EHK, QVL, SVL, NIV, NVI, ADM, SAA, SQH, NQV, ALL, ATM, QYA, STT, DGR, NHT, SHT, QHT, TTL, NHM, QHL, DNR, QHM, QFM, QMI, AHV, THI, AHT, TNH, NHV, THV, SHI, ATV, TWI, NYV, TYI, DYR, SFI, NQF, NWM, NSL, NSP, TQY, DRS, ASP, TKD, QWI, QWN, TWN, QWV, THN, TTM, TQQ, NMQ, TYA, STQ, STN, AGN, EKH, ERT, TFH, SFH, AFH, ENK, NYQ, SFM, QGS, AHF, QLT, QHY, AQY, TRD, ERH, QWH, TYH, TRE, TAQ, AER, TDR, ASH, EKI, AFV, TYN, ALT, NGN, SYY, EWN, NGM, NAN, TAS, TFV, SFV, QAN, SGT, SAY, AFQ, TFQ, QFQ, EFK, SYH, QHV, AMM, THQ, AAI, AAM, NSQ, QAT, TSQ, SHQ, QSM, AGV, AGT, TGT, TGI, AVI, TGV, SGI, ESP, SDA, TPN, EAW, NEQ, DSN, DIK, DNA, QES, SLQ, EKD, SDH, SSF, TET, QDI, DSA, DNM, DKT, DGM, SQY, TLT, QEH, DKQ, TGE, DNS, SDV, SNE, EGT, EGV, AAP, AHG, TWK, TQA, TVT, APQ, SVT, NGT, SSH, NLV, AGY, QFF, QYF, NWV, EKA, EKY, SMG, NAG, ASG, TFI, ELK, SEK, QTL, NML, DHS, ETR, NQQ, QWL, ANP, AHH, NEK, NNN, SNA, EWR, ESR, SHN, SFG, SYA, APV, QPV, SPT, TPQ, QVI, TPV, AVL, SEM, NDM, API, QLM, QTM, QQL, QHI, NFL, AYV, NKE, TIM, QYV, SWI, DRN, AWI, SMA, NRE, QTS, QVV, QQT, QMA, QQS, QAA, ATQ, ERL, TGS, QQQ, AQQ, QHF, TAN, QSY, QSN, NGQ, TWH, TMS, QMS, TON, TMT, QTV, THS, TTT, SMT, QMT, SMR, ADA, AAE, EPY, EYL, NWI, TSP, DGQ, NDH, QFD, EHL, ERD, DAF, ENI, ENH, QQW, AGM, ENL, EAF, EYV, ENT, SSE, AGE, QEY, DRH, NIQ, SVV, SVM, QPT, TVL, QTQ, SVQ, TIQ, NLQ, SIQ, AWL, EHH, NGY, NHF, NSA, NAA, NTH, QLI, NWH, QRE, AML, EVR, SNP, NHS, THH, NOS, TTS, AHS, EKV, NFQ, APL, QPM, NPV, SIL, TII, STV, QEL, TFL, SIT, NAQ, TMM, AAL, QQM, AMI, QFL, AHI, SYI, SSP, NVV, TTN, TAA, TOS, TMA, ALA, QQN, NSH, TTH, SRE, ERQ, QHS, SWH, SEV, AVT, ALQ, DKH, AMQ, ARE, TWV, NGL, NGV, TMQ, TLV, EER, and QTT.

98. The modified AAV capsid protein of any one of claims 94-97, wherein X₁X₂X₃ is selected from ANY, SNI, AAI, AAM, ANT, AST, AYQ, EHK, ENK, ENR, SFQ, SSI, TAY, TDK, TNT, AAF, AAL, AAY, ADK, AFA, ANF, ANI, ANQ, ANS, AQM, ARE, ASV, AYH, AYT, EMK, EWK, NNM, QAF, QAI, QAM, QAT, QAY, QFT, QGM, QHL, QNF, QNQ, QNS, QNT, QNV, QNY, SAH, SAI, SAL, SFT, SFV, SHI, SHV, SMM, SNF, SNM, SNN, SNQ, SNV, SNY, SQI, SQV, SSL, SWQ, SWS, SYI, SYM, SYN, SYQ, TAM, TAT, TDR, TFM, THV, TNF, TNH, TNI, TNM, TNQ, TNV, TSY, TWA, and TYM.

99. The modified AAV capsid protein of any one of claims 1-55, wherein X₇ is selected from F and Y; X₈ is selected from N and Q; X₉ is selected from N and S; X₁₀ is selected from T and L.

100. The modified AAV capsid protein of claim 99: wherein the targeting peptide has an amino acid sequence selected from: X₁X₂X₃RGDFNNT (SEQ ID NO: 98943), X₁X₂X₃RGDFNNL (SEQ ID NO: 98933), X₁X₂X₃RGDFQNT (SEQ ID NO: 98934) or X₁X₂X₃RGDYNSL (SEQ ID NO: 98940).

101. The modified AAV capsid protein of claim 99 or 100, wherein X₁ is S, A or E, X₂ is N, Y or S, X₃ is I, Q, R, V, T, M or K.

102. The modified AAV capsid protein of any one of claims 99-101, wherein X₁X₂X₃ is selected from the group consisting of: DMK, ATD, EEK, QMD, EFS, ERD, DDR, TDM, SAE, EHS, ENH, SWE, SNE, NNG, QAG, ERG, QSG, QNG, ASG, QFG, AMF, ELR, NFM, NNS, NNI, SDR, EQR, EHR, EWR, EQK, ESR, EKQ, EYR, ENR, EMK, EYK, EHK, EWK, QNI, TNI, TYI, SNY, DRQ, AWI, QWI, DFR, EKG, QYG, QWQ, EKN, EKF, EKT, AFH, ENK, NYS, DKH, AAG, QMF, QFH, QKD, ARE, AHQ, ADK, ADR, AHN, QNS, ANN, SWS, EKS, AFS, QFS, TNQ, TGY, NGL, ARD, AKD, AAF, SMF, AFN, SGT, TGL, ASN, SFQ, AFQ, TFQ, QFQ, EFK, SWQ, AYQ, SYQ, DQR, DMR, ASM, ANT, SHQ, TSL, SNF, QSL, ASI, SGL, SGI, NDA, DHQ, DFK, DTK, DNA, SDS, TDH, DGL, QDV, SDM, DKT, DGM, SQY, DKS, SSD, EMV, TSE, EYV, SSE, EGM, SDF, TTG, QLG, NNP, AGS, QSF, TLM, NGA, AWY, NKD, SWY, TFF, NSG, NGY, QEK, SEK, TEK, AQA, NVL, NHI, TER, NML, NMA, NNA, NHN, ANP, AMS, NER, SER, DKK, NMM, QDK, AHS, TNA, THT, SNH, QNH, ESK, SHS, AYM, SSA, NAT, QLM, QQM, SQI, SNI, QNV, SHV, SHM, NNV, TNN, AHT, TNH, NHV, TQM, THV, SHI, NYL, NMV, NFL, AYV, NQI, NQL, NMI, NSS, SHF, SYM, NYV, AYI, SWI, QFI, ANF, TFT, TWM, QNW, NSY, THY, TNY, NYN, AWN, SMA, NSH, TGH, SFH, SWH, SFM, NFT, QYH, QGS, TGS, TSA, QQQ, QYY, AMQ, TAN, AWH, NMS, TSN, TAH, TMH, QWH, NWS, SWN, QRD, TYH, AKE, TAQ, AER, AYH, ASA, SNS, QSS, SAH, SMH, ASH, QAS, ANS, SYS, QYN, TYN, TNS, SFT, ANQ, SGS, SGH, SSS, TWY, QTY, SYY, NGM, TMS, SYG, TON, SYN, TFN, TMN, AYN, SGN, QNN, SMN, SAN, QAF, ASF, ASY, TAS, TAF, TFV, SMQ, SFV, TMT, QAN, SAY, EDK, AMV, QHV, THS, TAT, QGI, QQV, NAV, SAV, ASV, QSV, QAV, AAI, AAM, QAM, TAM, DTR, NNT, NYT, NMT, ATT, QTT, TTT, QAT, SMV, SNT, QNT, AAT, SAM, SAL, QGV, QST, QYT, TSM, ANH, SNV, TSQ, TNT, SST, AST, TAI, TSI, TSV, TDK, SAT, QSM, SAI, QSI, SSV, SSI, AGT, TGT, AGI, QGM, TGM, TGV, SGM, SGV, SQE, EHT, EYL, DSG, SFE, QND, TQA, AWV, NFV, TSG, SSG, DRG, ANW, TFI, TSF, QNL, STL, TWL, NNF, NND, NYM, NFI, ETR, SNP, EIR, NFS, NNH, NNL, AHL, NNQ, NDR, NNN, QDR, SDK, SNA, AFG, NYA, QHA, QFA, SHN, NFQ, SYA, TFA, EMR, EFR, EAR, NOM, TTL, TQL, SMM, SKE, SYL, ASL, SLV, TTV, AAL, NHL, NHM, QYL, QHL, ATL, DNR, DSR, ANL, SHL, SNL, TNL, QAL, AMI, ANI, QNM, QYM, QHM, QFM, QMM, QMI, ANM, AHM, SQM, AQT, EKL, NNM, TNM, SNM, QQI, TQV, TQI, THI, QHI, THL, SNN, TNV, SWL, AQM, SYV, TFM, TYM, TWI, AFI, ATI, DYR, QLV, TMI, SYI, SFI, NWM, NWT, NSL, STF, SNW, AFM, SSY, NSV, TNF, NAF, TFS, QWV, ERT, TQT, AFT, NYQ, ERL, NRD, TRD, AWQ, TDR, TSH, ASS, AYT, TYS, QYS, SYT, SWV, SNQ, SMS, NAS, SAS, QFV, SAF, TWQ, TAV, AMM, TYT, QAI, TST, DAR, SSM, TAL, NES, DWK, DLR, DIR, SDT, EAN, ETH, TYD, SYE, EAM, TND, AYD, TSP, DRT, NDH, SSF, EFH, DNQ, DSH, DNT, SDI, DKQ, EWT, EAL, EAS, ESL, ASD, EAH, ASE, EAF, ENT, SIY, QDY, APG, AIG, SSW, NPG, ERP, TVI, QSH, SLS, EDR, AWL, SLH, EQH, EHH, NWV, EKY, STY, DRI, EYH, DRV, SPY, NNY, DWR, ERA, QRE, EVR, SRD, AWA, SFA, QTI, SHT, THM, AQI, AHI, SWT, ATV, TTI, SMI, QYV, NMY, SWM, QNY, TWT, QWT, TQY, NRE, QTS, QHQ, DVR, AAA, TYA, EKH, ALH, AWT, ATH, SRE, TFH, QMH, ERV, ERS, QHY, ALQ, QSY, ERH, TRE, TSS, QAH, AAH, SWA, TWS, QWS, AYS, AFV, QNQ, ANY, ALT, SMY, TWV, TTY, AMY, QMY, ERY, TWH, TSY, TAY, TYV, QAY, AAY, QYQ, TYQ, SYH, AQV, EER, THQ, and SMT.

103. The modified AAV capsid protein of any one of claims 99-102, wherein X₁X₂X₃ is selected from the group consisting of: ADR, ASI, EFK, EHK, EWK, SYQ, AAF, AAT, AAY, AFI, AFQ, AGI, AGT, AHI, ANH, ANM, ANN, AQI, ASA, ASH, AST, ASV, AWT, AYQ, AYT, DAR, DMK, DQR, DVR, EAR, EFR, EMK, EMR, EQK, ERA, ERS, ESR, NDA, NDR, NMI, NMV, NNM, NNN, NYL, NYM, NYN, NYQ, NYV, QAM, QFQ, QFV, QGV, QNH, QNI, QNM, QQV, QSF, QSY, SAI, SAM, SAS, SDR, SFQ, SGH, SGM, SGT, SGV, SHL, SHM, SHQ, SHV, SMH, SNA, SNE, SNF, SNI, SNQ, SNT, SRE, SST, SWQ, SWT, SYG, SYY, TFQ, THL, THQ, THV, TMI, TNL, TNS, TSH, TSQ, and TWQ.

104. The modified AAV capsid protein of any one of claims 1-55, wherein RGDRX₈X₉X₁₀ has an amino acid sequence selected from: RGDRGVX₁₀ (SEQ ID NO: 157058), RGDRGSX₁₀ (SEQ ID NO: 157059), RGDRGNX₁₀ (SEQ ID NO: 157060), RGDRGGX₁₀ (SEQ ID NO: 157061), RGDRGQX₁₀ (SEQ ID NO: 157062), RGDRGX₉V (SEQ ID NO: 157063), RGDRGX₉I (SEQ ID NO: 157064), RGDRGX₉S (SEQ ID NO: 157065), RGDRGX₉L (SEQ ID NO: 157066), RGDRGX₉Q (SEQ ID NO: 157067), RGDHX₈X₉L (SEQ ID NO: 157068), RGDRX₈X₉I (SEQ ID NO: 157069), RGDRX₈X₉V (SEQ ID NO: 157070), RGDRX₈X₉L (SEQ ID NO: 157071), RGDYX₈X₉L (SEQ ID NO: 157072), RGDYX₈X₉V (SEQ ID NO: 157073), RGDYX₈X₉M (SEQ ID NO: 157074), and RGDLX₈X₉T (SEQ ID NO: 157075).

105. The modified AAV capsid protein of any one of claims 1-104, wherein the modified sequence does not comprise an amino acid sequence selected from: RGDRMVF (SEQ ID NO: 157080), RGDRTVI (SEQ ID NO: 157081), SRGDRPM (SEQ ID NO: 157082), ISLRGDR (SEQ ID NO: 157083), and RGDLLLS (SEQ ID NO: 1).

106. The modified AAV capsid protein of any one of claims 1-105, wherein the targeting peptide has a sequence selected from: X₁X₂X₃RGDHVNL (SEQ ID NO: 98924); X₁X₂X₃RGDLIGR (SEQ ID NO: 98925); X₁X₂X₃RGDQSTL (SEQ ID NO: 98926); X₁X₂X₃RGDRGQI (SEQ ID NO: 98927); X₁X₂X₃RGDRGVV (SEQ ID NO: 98928); X₁X₂X₃RGDRQGI (SEQ ID NO: 98929); X₁X₂X₃RGDRSQT (SEQ ID NO: 98930); X₁X₂X₃RGDRSVV (SEQ ID NO: 98931); X₁X₂X₃RGDLLLS (SEQ ID NO: 98932); X₁X₂X₃RGDFNNL (SEQ ID NO: 98933); X₁X₂X₃RGDFQNT (SEQ ID NO: 98934); X₁X₂X₃RGDLTVT (SEQ ID NO: 98935); X₁X₂X₃RGDYVGL (SEQ ID NO: 98936); X₁X₂X₃RGDYSSV (SEQ ID NO: 98937); X₁X₂X₃RGDHGVL (SEQ ID NO: 98938); X₁X₂X₃RGDRDYL (SEQ ID NO: 98939); X₁X₂X₃RGDYNSL (SEQ ID NO: 98940); X₁X₂X₃RGDYTSV (SEQ ID NO: 98941); X₁X₂X₃RGDYTSM (SEQ ID NO: 98942); and X₁X₂X₃RGDFNNT (SEQ ID NO: 98943).

107. The modified AAV capsid protein of any one of claims 1-106, wherein the targeting peptide is positioned between 565 and 595 within VR VIII of the modified AAV capsid protein.

108. The modified AAV capsid protein of any one of claims 1-107, wherein:
the reference AAV capsid protein is a capsid protein of AAV1 or a modification thereof and the targeting peptide is between Q585 and T589 of the reference AAV capsid protein;
the reference AAV capsid protein is a capsid protein of AAV2 or a modification thereof and the targeting peptide is between Q584 and R588 or between N587 and R588 of the reference AAV capsid protein;
the reference AAV capsid protein is a capsid protein of AAV3 or a modification thereof and the targeting peptide is between Q585 and T589 of the reference AAV capsid protein;
the reference AAV capsid protein is a capsid protein of AAV4 or a modification thereof and the targeting peptide is between G581 and N585 of the reference AAV capsid protein;
the reference AAV capsid protein is a capsid protein of AAV5 or a modification thereof and the targeting peptide is between Q574 and T578 of the reference AAV capsid protein;
the reference AAV capsid protein is a capsid protein of AAV6 or a modification thereof and the targeting peptide is between Q585 and T589 of the reference AAV capsid protein;
the reference AAV capsid protein is a capsid protein of AAV7 or a modification thereof and the targeting peptide is between (2586 and T590 of the reference AAV capsid protein;
the reference AAV capsid protein is a capsid protein of AAV8 or a modification thereof and the targeting peptide is between Q587 and A591 of the modified AAV capsid protein;
the reference AAV capsid protein is a capsid protein of AAV9 or a modification thereof and the targeting peptide is between H587 and A591 of the reference AAV capsid protein;
the reference AAV capsid protein is a capsid protein of AAVrh10 or a modification thereof and the targeting peptide is between Q587 and A591 of the reference AAV capsid protein;
the reference AAV capsid protein is a capsid protein of AAVpo.1 or a modification thereof and the targeting peptide is between N564 and S568 of the reference AAV capsid protein;
the reference AAV capsid protein is a capsid protein of AAV12 or a modification thereof and the targeting peptide is between N589 and A593 of the reference AAV capsid protein;
the reference AAV capsid protein is a capsid protein of Anc80 or a modification thereof and the targeting peptide is between Q585 and T589 of the reference AAV capsid protein;
the reference AAV capsid protein is a capsid protein of Anc80L65 or a modification thereof and the targeting peptide is between Q585 and T589 of the reference AAV capsid protein;
the reference AAV capsid protein is a capsid protein of Anc80-55 or a modification thereof and the targeting peptide is between Q585 and T589 of the reference AAV capsid protein;
the reference AAV capsid protein is a capsid protein of Anc80-129 or a modification thereof and the targeting peptide is between Q585 and T589 of the reference AAV capsid protein;
the reference AAV capsid protein is a capsid protein of Anc80-156 or a modification thereof and the targeting peptide is between Q585 and T589 of the reference AAV capsid protein;
the reference AAV capsid protein is a capsid protein of Anc80-751 or a modification thereof and the targeting peptide is between Q585 and T589 of the reference AAV capsid protein;
the reference AAV capsid protein is a capsid protein of Anc80-1029 or a modification thereof and the targeting peptide is between Q585 and T589 of the reference AAV capsid protein; or
the reference AAV capsid protein is a capsid protein of Anc80-1712 or a modification thereof and the targeting peptide is between Q585 and T589 of the reference AAV capsid protein.

109. The modified AAV capsid protein of any one of claims 1-107, wherein:
the reference AAV capsid protein is a capsid protein of AAV1 or a modification thereof and the targeting peptide is between D590 and P591 or between S588 and T589 of the reference AAV capsid protein;
the reference AAV capsid protein is a capsid protein of AAV2 or a modification thereof and the targeting peptide is between R588 and Q589 or between N587 and R588 of the reference AAV capsid protein;
the reference AAV capsid protein is a capsid protein of AAV3 or a modification thereof and the targeting peptide is between S586 and S587 or between N588 and T589 of the reference AAV capsid protein;
the reference AAV capsid protein is a capsid protein of AAV4 or a modification thereof and the targeting peptide is between S584 and N585 or between S586 and N587 of the reference AAV capsid protein;
the reference AAV capsid protein is a capsid protein of AAV5 or a modification thereof and the targeting peptide is between S575 and S576 or between T577 and T578 of the reference AAV capsid protein;
the reference AAV capsid protein is a capsid protein of AAV6 or a modification thereof and the targeting peptide is between D590 and P591 or S588 and T589 of the reference AAV capsid protein;
the reference AAV capsid protein is a capsid protein of AAV7 or a modification thereof and the targeting peptide is between N589 and T590 of the reference AAV capsid protein;
the reference AAV capsid protein is a capsid protein of AAV8 or a modification thereof and the targeting peptide is between N590 and T591 of the modified AAV capsid protein;
the reference AAV capsid protein is a capsid protein of AAV9 or a modification thereof and the targeting peptide is between Q588 and A589 of the reference AAV capsid protein;
the reference AAV capsid protein is a capsid protein of AAVrh10 or a modification thereof and the targeting peptide is between N590 and A591 of the reference AAV capsid protein;
the reference AAV capsid protein is a capsid protein of AAVpo.1 or a modification thereof and the targeting peptide is between N567 and S568 or between N569 and T570 of the reference AAV capsid protein;
the reference AAV capsid protein is a capsid protein of AAV12 or a modification thereof and the targeting peptide is between N592 and A593 or between T594 and T595 of the reference AAV capsid protein;
the reference AAV capsid protein is a capsid protein of Anc80 or a modification thereof and the targeting peptide is between T589 and A590 or between N587 and T588 of the reference AAV capsid protein;
the reference AAV capsid protein is a capsid protein of Anc80L65 or a modification thereof and the targeting peptide is between T589 and A590 or between N587 and T588 of the reference AAV capsid protein;
the reference AAV capsid protein is a capsid protein of Anc80-55 or a modification thereof and the targeting peptide is between T589 and A590 or between N587 and T588 of the reference AAV capsid protein;
the reference AAV capsid protein is a capsid protein of Anc80-129 or a modification thereof and the targeting peptide is between T589 and A590 or between N587 and T588 of the reference AAV capsid protein;
the reference AAV capsid protein is a capsid protein of Anc80-156 or a modification thereof and the targeting peptide is between T589 and A590 or between N587 and T588 of the reference AAV capsid protein;
the reference AAV capsid protein is a capsid protein of Anc80-751 or a modification thereof and the targeting peptide is between T589 and A590 or between N587 and T588 of the reference AAV capsid protein;
the reference AAV capsid protein is a capsid protein of Anc80-1029 or a modification thereof and the targeting peptide is between T589 and A590 or between N587 and T588 of the reference AAV capsid protein; or
the reference AAV capsid protein is a capsid protein of Anc80-1712 or a modification thereof and the targeting peptide is between T589 and A590 or between N587 and T588 of the reference AAV capsid protein.

110. The modified AAV capsid protein of any one of claims 1-109, wherein
(i) P₁ is independently selected from an asparagine (N), a serine (S), or a threonine (T);
(ii) P₂ is independently selected from a serine (S) or a glycine (G);
(iii) P₃ is independently selected from a threonine (T), a glutamine (Q), an alanine (A), or glutamate (E);
(iv) P₄ is independently selected from a serine (S), a threonine (T), or an alanine (A);
(v) P₅ is independently selected from a glycine (G) or an alanine (A);
(vi) P₆ is independently selected from a glycine (G) or an alanine (A);
(vii) P₇ is independently selected from an alanine (A) or a serine (S);
(viii) P₈ is independently selected from a serine (S) or a threonine (T); and
(ix) P₁₂ is independently selected from a histidine (H), a threonine (T), or an alanine (A).

111. The modified AAV capsid protein of claim 110, wherein P₅, P₆ or both P₅ and P₆ are not an alanine (A).

112. The modified AAV capsid protein of claim 110, wherein the peptide segment does not comprise an alanine (A) at P₆ and a threonine (T) at P₈.

113. The modified AAV capsid protein of any one of claims 1-112, wherein the peptide segment has a sequence of P₁P₂P₃P₄GGP₇P₈NDNP₁₂ (SEQ ID NO: 44921), wherein P₁, P₂, P₃, P₄, P₇, P₈, and P₁₂ are independently selected from any amino acid residue.

114. The modified AAV capsid protein of claim 113, wherein
(i) P₁ is independently selected from an asparagine (N) or a serine (S);
(ii) P₂ is independently selected from a serine (S) or a glycine (G);
(iii) P₃ is independently selected from a threonine (T) or a glutamine (Q);
(iv) P₄ is independently selected from a serine (S), a threonine (T), or an alanine (A);
(v) P₇ is independently selected from an alanine (A) or a serine (S);
(vi) P₈ is independently selected from a serine (S) or a threonine (T); and
(vii) P₁₂ is independently selected from a histidine (H), a threonine (T), or an alanine (A).

115. The modified AAV capsid protein of any claim one of claims 1-113, wherein the peptide segment has a sequence of P₁P₂TP₄GGP₇P₈NDNP₁₂ (SEQ ID NO: 44922), wherein P₁, P₂, P₄, P₇, P₈, and P₁₂ are independently selected from any amino acid residue.

116. The modified AAV capsid protein of any one of claims 1-113, wherein the peptide segment has a sequence of P₁P₂QP₄GGP₇P₈NDNP₁₂ (SEQ ID NO: 44923), wherein P₁, P₂, P₄, P₇, P₈, and P₁₂ are independently selected from any amino acid residue.

117. The modified AAV capsid protein of any one of claims 1-113, wherein the peptide segment has a sequence of P₁P₂TP₄GGP₇TNDNP₁₂ (SEQ ID NO: 44924), wherein P₁, P₂, P₄, P₇, and P₁₂ are independently selected from any amino acid residue.

118. The modified AAV capsid protein of any one of claims 1-113, wherein the peptide segment has a sequence of NP₂TP₄GGP₇P₈NDNP₁₂ (SEQ ID NO: 44925), wherein P₂, P₄, P₇, and P₁₂ are independently selected from any amino acid residue.

119. The modified AAV capsid protein of any one of claims 1-113, wherein the peptide segment has a sequence of SP₂TP₄GGP₇P₈NDNP₁₂ (SEQ ID NO: 44926), wherein P₂, P₄, P₇, P₈, and P₁₂ are independently selected from any amino acid residue.

120. The modified AAV capsid protein of any one of claims 1-113, wherein the peptide segment is NSTSGGP₇P₈NDNH (SEQ ID NO: 44927), wherein P₇ and P₈ are independently selected from any amino acid residue.

121. The modified AAV capsid of protein of claim 120, wherein P₇ is independently selected from an alanine (A) or a serine (S) and P₈ is independently selected from a serine (S) or a threonine (T).

122. The modified AAV capsid protein of claim 120 or 121, wherein the peptide segment comprises:
(i)
(SEQ ID NO: 46026)
NSTSGGASNDNH,
(ii)
(SEQ ID NO: 46029)
NSTSGGATNDNH,
(iii)
(SEQ ID NO: 46031)
NSTSGGSSNDNH,
or
(iv)
(SEQ ID NO: 46034)
NSTSGGSTNDNH.

123. The modified AAV capsid protein of any one of claims 1-113, wherein the peptide segment is NSTTGGP₇P₈NDNH (SEQ ID NO: 44928), wherein P₇ and P₈ are independently selected from any amino acid residue.

124. The modified AAV capsid protein of claim 123, wherein P₇ is independently selected from an alanine (A) or a serine (S) and P₈ is independently selected from a serine (S) or a threonine (T).

125. The modified AAV capsid protein of claim 123 or 124, wherein the peptide segment comprises:
(i)
(SEQ ID NO: 46073)
NSTTGGASNDNH,
(ii)
(SEQ ID NO: 46076)
NSTTGGATNDNH,
(iii)
(SEQ ID NO: 46079)
NSTTGGSSNDNH,
or
(iv)
(SEQ ID NO: 46082)
NSTTGGSTNDNH.

126. The modified AAV capsid protein of any one of claims 1-113, wherein the peptide segment is SGQTGGP₇P₈NDNH (SEQ ID NO: 44929), wherein P₇ and P₈ are independently selected from any amino acid residue.

127. The modified AAV capsid protein of claim 126, wherein P₇ is independently selected from an alanine (A) or a serine (S) and P₈ is independently selected from a serine (S) or a threonine (T).

128. The modified AAV capsid protein of claim 126 or 127, wherein the peptide segment comprises:
(i)
(SEQ ID NO: 46505)
SGQTGGASNDNH,
(ii)
(SEQ ID NO: 46508)
SGQTGGATNDNH,
(iii)
(SEQ ID NO: 46511)
SGQTGGSSNDNH,
or
(iv)
(SEQ ID NO: 46514)
SGQTGGSTNDNH.

129. The modified AAV capsid protein of any one of claims 1-113, wherein the peptide segment is SGTAGGP₇P₈NDNT (SEQ ID NO: 44930), wherein P₇ and P₈ are independently selected from any amino acid residue.

130. The modified AAV capsid protein of claim 129, wherein P₇ is independently selected from an alanine (A) or a serine (S) and P₈ is independently selected from a serine (S) or a threonine (T).

131. The modified AAV capsid protein of claim 129 or 130, wherein the peptide segment comprises:
(i)
(SEQ ID NO: 46554)
SGTAGGASNDNT,
or
(ii)
(SEQ ID NO: 46560)
SGTAGGSSNDNT.

132. The modified AAV capsid protein of claim 129 or 130, wherein the peptide segment does not comprise SGTAGGATNDNT (SEQ ID NO: 46557) or SGTAGGSTNDNT (SEQ ID NO: 46563).

133. The modified AAV capsid protein of any one of claims 1-113, wherein the peptide segment is SGTSGGP₇P₈NDNA (SEQ ID NO: 44931), wherein P₇ and P₈ are independently selected from any amino acid residue.

134. The modified AAV capsid protein of claim 133, wherein P₇ is independently selected from an alanine (A) or a serine (S) and P₈ is independently selected from a serine (S) or a threonine (T).

135. The modified AAV capsid protein of claim 133 or 134, wherein the peptide segment comprises:
(i)
(SEQ ID NO: 46600)
SGTSGGASNDNA,
(ii)
(SEQ ID NO: 46603)
SGTSGGATNDNA,
(iii)
(SEQ ID NO: 46606)
SGTSGGSSNDNA,
or
(iv)
(SEQ ID NO: 46609)
SGTSGGSTNDNA.

136. The modified AAV capsid protein of any one of claims 1-113, wherein the peptide segment is SGTTGGP₇P₈NDNT (SEQ ID NO: 44932), wherein P₇ and P₈ are independently selected from any amino acid residue.

137. The modified AAV capsid protein of claim 136, wherein P₇ is independently selected from an alanine (A) or a serine (S) and P₈ is independently selected from a serine (S) or a threonine (T).

138. The modified AAV capsid protein of claim 136 or 137, wherein the peptide segment comprises:
(i)
(SEQ ID NO: 46650)
SGTTGGASNDNT,
(ii)
(SEQ ID NO: 46653)
SGTTGGATNDNT,
(iii)
(SEQ ID NO: 46656)
SGTTGGSSNDNT,
or
(iv)
(SEQ ID NO: 46659)
SGTTGGSTNDNT.

139. The modified AAV capsid protein of claim 138, wherein the peptide segment is SGTTGGSSNDNT (SEQ ID NO: 46656).

140. The modified AAV capsid protein of any one of claims 1-113, wherein the peptide segment is SSTAGGP₇P₈NDNA (SEQ ID NO: 44933), wherein P₇ and P₈ are independently selected from any amino acid residue.

141. The modified AAV capsid protein of claim 139, wherein P₇ is independently selected from an alanine (A) or a serine (S) and P₈ is independently selected from a serine (S) or a threonine (T).

142. The modified AAV capsid protein of claim 140 or 141, wherein the peptide segment comprises:
(i)
(SEQ ID NO: 47128)
SSTAGGASNDNA,
(ii)
(SEQ ID NO: 47131)
SSTAGGATNDNA,
(iii)
(SEQ ID NO: 47134)
SSTAGGSSNDNA,
or
(iv)
(SEQ ID NO: 47137)
SSTAGGSTNDNA.

143. The modified AAV capsid protein of claim 142, wherein the peptide segment is SSTAGGASNDNA (SEQ ID NO: 47128).

144. The modified AAV capsid protein of claim 142, wherein the peptide segment is SSTAGGATNDNA (SEQ ID NO: 47131).

145. The modified AAV capsid protein of any one of claims 1-113, wherein the peptide segment is NSTSGASTNDNA (SEQ ID NO: 48390).

146. The modified AAV capsid protein of any one of claims 1-113, wherein the peptide segment is selected from a peptide segment as shown in Tables 20 or 28.

147. The modified AAV capsid protein of any one of claims 1-146, wherein variable region 1 (VR I) corresponds to amino acid residues between about position 259 to about position 275 of the modified capsid protein.

148. The modified AAV capsid protein of any one of claims 1-146, wherein the peptide segment is at a position between S261 and Y274 of an AAV9 capsid protein (SEQ ID NO: 61).

149. The modified AAV capsid protein of any one of claims 1-146, wherein the peptide segment is at a position between S260 and Y273 of an Anc80 capsid protein (SEQ ID NO. 132).

150. The modified AAV capsid protein of any one of claims 1-146, wherein the peptide segment is at a position between S260 and Y273 of an Anc80L65 capsid protein (SEQ ID) NO: 142).

151. The modified AAV capsid protein of any one of claims 1-146, wherein the peptide segment is at a position between S260 and Y273 of an AAV2 capsid protein (SEQ ID NO: 55).

152. The modified AAV capsid protein of any one of claims 1-151, wherein
(i) the targeting peptide has an amino acid sequence selected from SEQ ID NOs: 44864-44867, 44879-44883, 44911, 44912, 44913, 44918-44919, and 48391-157057; and
(ii) the peptide segment has an amino acid sequence selected from SEQ ID NOs: 46026, 46029, 46031, 46035, 46073, 46076, 46079, 46082, 46505, 46508, 46511, 46514, 46554, 46560, 46609, 46600, 46603, 46606, 46650, 46653, 46656, 46659, 47128, 47131, 47134, 47137, and 48390.

153. The modified AAV capsid protein of any one of claims 1-152, wherein
(i) the targeting peptide has an amino acid sequence selected from SEQ ID NOs: 44864-44867, 44879-44883, 44911, 44912, 44913, 44918-44919, and 48391-157057; and
(ii) the peptide segment has the amino acid sequence SGTAGGASNDNT (SEQ ID NO: 46554).

154. The modified AAV capsid protein of any one of claims 1-152, wherein
(i) the targeting peptide has an amino acid sequence selected from SEQ ID NOs: 44864-44867, 44879-44883, 44911, 44912, 44913, 44918-44919, and 48391-157057; and
(ii) the peptide segment has the amino acid sequence SGTSGGSTNDNA (SEQ ID NO: 46609).

155. The modified AAV capsid protein of any one of claims 1-152, wherein
(i) the targeting peptide has an amino acid sequence selected from SEQ ID NOs: 44864-44867, 44879-44883, 44911, 44912, 44913, 44918-44919, and 48391-157057; and
(ii) the peptide segment has the amino acid sequence SGTTGGSTNDNT (SEQ ID NO: 46659).

156. The modified AAV capsid protein of any one of claims 1-152, wherein
(i) the targeting peptide has an amino acid sequence selected from SEQ ID NOs: 44864-44867, 44879-44883, 44911, 44912, 44913, 44918-44919, and 48391-157057; and
(ii) the peptide segment has the amino acid sequence SGTTGGSTNDNT (SEQ ID NO: 47128).

157. The modified AAV capsid protein of any one of claims 1-152, wherein
(i) the targeting peptide has an amino acid sequence selected from SEQ ID NOs: 44864-44867, 44879-44883, 44911, 44912, 44913, 44918-44919, and 48391-157057; and
(ii) the peptide segment has the amino acid sequence NSTSGGSSNDNA (SEQ ID NO: 48388).

158. The modified AAV capsid protein of any one of claims 1-152, wherein
(i) the targeting peptide has an amino acid sequence selected from SEQ ID NOs: 44864-44867, 44879-44883, 44911, 44912, 44913, 44918-44919, and 48391-157057; and
(ii) the peptide segment has the amino acid sequence NSTSGASTNDNA (SEQ ID NO: 48390).

159. The modified AAV capsid protein of any one of claims 1-152, wherein
(i) the targeting peptide has the amino acid sequence ENRRGDFNNL (SEQ ID NOS: 44864); and
(ii) the peptide segment has the amino acid sequence SGTTGGSTNDNT (SEQ ID NO: 46659).

160. The modified AAV capsid protein of any one of claims 1-152, wherein
(i) the targeting peptide has the amino acid sequence ENRRGDENNL (SEQ ID NOs: 44864); and
(ii) the peptide segment has the amino acid sequence SGTTGGSSNDNT (SEQ ID NO: 46656).

161. The modified AAV capsid protein of any one of claims 1-152, wherein
(i) the targeting peptide has the amino acid sequence ENRRGDFNNL (SEQ ID NOS: 44864); and
(ii) the peptide segment has the amino acid sequence SSTAGGASNDNA (SEQ ID NO: 47128).

162. The modified AAV capsid protein of any one of claims 1-152, wherein
(i) the targeting peptide has the amino acid sequence ENRRGDFNNL (SEQ ID NOs: 44864); and
(ii) the peptide segment has the amino acid sequence NSTSGASTNDNA (SEQ ID NO: 48390).

163. The modified AAV capsid protein of any one of claims 1-152, wherein
(i) the targeting peptide has the amino acid sequence SAQRGDRGQI (SEQ ID NO: 44911); and
(ii) the peptide segment has the amino acid sequence SSTAGGATNDNA (SEQ ID NO: 47131).

164. The modified AAV capsid protein of any one of claims 1-152, wherein
(i) the targeting peptide has the amino acid sequence SAQRGDRGQI (SEQ ID NO: 44911); and
(ii) the peptide segment has the amino acid sequence SGTTGGSTNDNT (SEQ ID NO: 46659).

165. The modified AAV capsid protein of any one of claims 1-152, wherein
(i) the targeting peptide has the amino acid sequence SAQRGDRGQI (SEQ ID NO: 44911); and
(ii) the peptide segment has the amino acid sequence SGTTGGSSNDNT (SEQ ID NO: 46656).

166. The modified AAV capsid protein of any one of claims 1-152, wherein
(i) the targeting peptide has the amino acid sequence SAQRGDRGQI (SEQ ID NO: 44911); and
(ii) the peptide segment has the amino acid sequence SSTAGGASNDNA (SEQ ID NO: 47128).

167. The modified AAV capsid protein of any one of claims 1-152, wherein
(i) the targeting peptide has the amino acid sequence ENRRGDENNL (SEQ ID NOs: 44864); and
(ii) the peptide segment has the amino acid sequence NSTSGASTNDNA (SEQ ID NO: 48390).

168. The modified AAV capsid protein of any one of claims 1-152, wherein
(i) the targeting peptide has the amino acid sequence ENRRGDFQNT (SEQ ID NO: 44866); and
(ii) the peptide segment has the amino acid sequence SSTAGGATNDNA (SEQ ID NO: 47131).

169. The modified AAV capsid protein of any one of claims 1-152, wherein
(i) the targeting peptide has the amino acid sequence ENRRGDFQNT (SEQ ID NO: 44866); and
(ii) the peptide segment has the amino acid sequence SGTTGGSTNDNT (SEQ ID NO: 46659).

170. The modified AAV capsid protein of any one of claims 1-152, wherein
(i) the targeting peptide has the amino acid sequence ENRRGDFQNT (SEQ ID NO: 44866); and
(ii) the peptide segment has the amino acid sequence SGTTGGSSNDNT (SEQ ID NO: 46656).

171. The modified AAV capsid protein of any one of claims 1-152, wherein
(i) the targeting peptide has the amino acid sequence ENRRGDFQNT (SEQ ID NO: 44866); and
(ii) the peptide segment has the amino acid sequence NSTSGASTNDNA (SEQ ID NO: 48390).

172. The modified AAV capsid protein of any one of claims 1-152, wherein
(i) the targeting peptide has the amino acid sequence SNRRGDENNT (SEQ ID NO: 44883); and
(ii) the peptide segment has the amino acid sequence SSTAGGASNDNA (SEQ ID NO: 47128).

173. A modified adeno-associated virus (AAV) capsid protein, comprising:
(i) a targeting peptide at a site within VR VIII, wherein the targeting peptide has a sequence selected from SEQ ID NOs: 44864-44867, 44879-44883, 44911, 44912, 44913, and 44918-44919; and
(ii) a peptide segment within VR I, wherein the peptide segment has a sequence selected from SEQ ID NOs: 46026, 46029, 46031, 46035, 46073, 46076, 46079, 46082, 46505, 46508, 46511, 46514, 46554, 46560, 46609, 46600, 46603, 46606, 46650, 46653, 46656, 46659, 47128, 47131, 47134, and 47137.

174. A polynucleotide encoding the modified AAV capsid protein of any one of claims 1-173.

175. A vector comprising the polynucleotide of claim 174.

176. The vector of claim 175, further comprising a promoter operably linked to the polynucleotide.

177. A host cell comprising the modified AAV capsid protein of any one of claims 1 to 172, the polynucleotide specified in claim 174, or the vector of claim 175 or 176.

178. A recombinant AAV virion (rAAV) comprising the modified AAV capsid protein of any one of claims 1-173.

179. The rAAV virion of claim 178, further comprising an exogenous polynucleotide.

180. The rAAV virion of claim 179, wherein the exogenous polynucleotide comprises a template for homology directed repair.

181. The rAAV virion of claim 179, wherein the exogenous polynucleotide comprises an expressible polynucleotide encoding a therapeutic tRNA, miRNA, gene editing guide RNA, or RNA-editing guide RNA.

182. The rAAV virion of claim 179, wherein the exogenous polynucleotide comprises an expressible polynucleotide encoding a therapeutic protein.

183. The rAAV virion of claim 182, wherein the therapeutic protein is MTM1 or a fragment thereof.

184. The rAAV virion of claim 183, wherein the expressible polypeptide comprises the sequence of SEQ ID NO: 165 or a fragment thereof.

185. The rAAV virion of claim 183, wherein the expressible polypeptide comprises the sequence having at least 80%, 85%, 90%, 95%, 98%, 99% or 100% sequence identity to any of SEQ ID Nos: 166-170.

186. The rAAV virion of any one of claims 179-185, wherein the exogenous polynucleotide further comprises a regulatory sequence.

187. The rAAV virion of claim 186, wherein the regulatory sequence comprises expression regulatory elements (EREs).

188. The rAAV virion of claim 187, wherein the EREs comprise a CAG promoter.

189. The rAAV virion of claim 187, wherein the EREs comprise a sequence having at least 90%, at least 95%, at least 98%, at least 99% or 100% sequence identity to any one of SEQ IDs NO: 171-173.

190. A pharmaceutical composition comprising the modified AAV capsid protein of any one of claims 1-173 or the AAV virion of any one of claims 178-189.

191. A method for treating or ameliorating or preventing a disease or condition in a subject, comprising administering a therapeutically effective amount of the rAAV virion of any one of claims 178-189 or the pharmaceutical composition of claim 190.

192. The method of treating or ameliorating or preventing a disease according to claim 191, wherein the disease is a muscular disease and/or the condition is muscle degeneration.

193. The method of treating or ameliorating or preventing a disease according to claim 192, wherein said muscle is a striated muscle, preferably heart or a skeletal muscle or diaphragm.

194. The method of treating or ameliorating or preventing a disease according to claim 193, wherein said muscular disease is a muscular dystrophy, a cardiomyopathy, a myotonia, a muscular atrophy, a myoclonus dystonia, a mitochondrial myopathy, a rhabdomyolysis, a fibromyalgia, and/or a myofascial pain syndrome.

195. The modified adeno-associated virus (AAV) capsid protein of any one of claims 1-173, for use in treating and/or preventing a muscular disease and/or muscle degeneration.

196. An AAV virion comprising the modified AAV capsid protein of any one of claims 1-173 or the AAV virion of any one of claims 178-189 for use in treating and/or preventing a muscular disease and/or in muscle regeneration.

197. A pharmaceutical composition comprising the modified AAV capsid protein of any one of claims 1-159, and/or the AAV virion specified in any one of claims 178-189 for use in treating and/or preventing a muscular disease and/or in muscle regeneration.

198. A method of transferring an exogenous polynucleotide into a muscle cell, comprising the step of administering the AAV virion specified in any one of claims 178-189 to a subject.

199. The method of claim 198, wherein the administration results in transfer of the exogenous polynucleotide in the muscle cell, at a muscle:liver infection ratio of greater than 1 when measured by genome copies of the AAV virion.

200. The method of claim 198, wherein the muscle:liver infection ratio ranges from 1 to 100.

201. The method of claim 199, wherein the muscle:liver infection ration ranges from 1 to 10.

202. The method of claim 201, wherein the muscle:liver infection ratio ranges from 2 to 8.

203. The method of any one of claims 198-202, wherein the administration results in expression of the exogenous polynucleotide in the muscle cell, at a muscle:liver expression ratio of greater than 10.

204. The method of claim 203, wherein the muscle:liver expression ratio ranges from 10 to 100.

205. The method of claim 204, wherein the muscle:liver expression ratio ranges from 20 to 80.

206. The method of any one of claims 198-205, wherein the muscle:liver expression ratio ranges from 50 to 80 when measured by mRNA transcript expression.

207. The method of any one of claims 198-206, wherein the muscle:liver expression ratio ranges from 10 to 50 when measured by protein expression.

208. The method of any one of claims 198-207, wherein the muscle cell is selected from triceps surae, biceps, heart and quadricep.

209. Use of the AAV capsid polypeptide of any one of claims 1-173, and/or the AAV virion specified in any one of claims 178-189 for transferring an exogenous polynucleotide into a muscle cell.

210. The use according to claim 209, wherein said use is a non-therapeutic use, preferably wherein said use is an in vitro use.

211. The use according to claim 209, wherein the muscle cell is selected from triceps surae, biceps, heart and quadricep.
